SEQUENCE LISTING (1 sequence)

SEQ ID NO: 1 - protein, 470 aa, synthetic construct
  source 1..470: note = hPDL1(18-239)-TVMV-mIgG1(221-447)-C225S
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSPGGGGGRETVRFQGGTGDAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2026/0070945 A1
(43) Pub. Date: Mar. 12, 2026

(54) MACROCYCLIC IMMUNOMODULATORS

(71) Applicant: Bristol-Myers Squibb Company, Princeton, NJ (US)

(72) Inventors: Jennifer X. QIAO, Princeton, NJ (US); Martin Patrick ALLEN, Flemington, NJ (US); Tammy C. WANG, Lawrenceville, NJ (US); Yunhui ZHANG, Princeton, NJ (US); Claude A. QUESNELLE, Skillman, NJ (US); Michael A. POSS, Lawrenceville, NJ (US)

(21) Appl. No.: 19/110,794

(22) PCT Filed: Sep. 8, 2023
(86) PCT No.: PCT/US2023/073775

§ 371 Date: Mar. 11, 2025

Related U.S. Application Data
(60) Provisional application No. 63/375,334, filed on Sep. 12, 2022.

(51) Int. Cl. C07K 7/56; A61K 38/00; A61P 37/02

(52) U.S. Cl. CPC C07K 7/56; A61P 37/02; A61K 38/00

(57) ABSTRACT

In accordance with the present disclosure, macrocyclic compounds have been discovered that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims the priority benefit of U.S. Provisional Application No. 63/375,334, filed Sep. 12, 2022, which is incorporated by reference herein in its entirety.

### REFERENCE TO SEQUENCE LISTING SUBMITTED ELECTRONICALLY

The content of the electronically submitted sequence listing (Name 3338.265PC01_Seqlisting_ST26.xml; Size: 2,645 bytes; and Date of Creation: Sep. 7, 2023) filed with the application is incorporated herein by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med., 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med., 192:1027-1034 (2000); Latchman et al., Nat. Immunol., 2:261-268 (2001); Carter et al., Eur. J. Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med., 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med., 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99:12293-12297 (2002); Brown et al., J. Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26:Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med., 205(3):543-555 (2008); Finnefrock, A. C. et al., “PD-1 blockade in rhesus macaques: impact on chronic infection and prophylactic vaccination”, J. Immunol., 182(2):980-987 (2009); Song, M.-Y. et al., “Enhancement of vaccine-induced primary and memory CD8+ t-cell responses by soluble PD-1”, J. Immunother., 34(3):297-306 (2011).

The PD-1 pathway is a key inhibitory mechanism in T cell exhaustion that arises from chronic antigen stimulation during tumor disease. Accordingly, agents that block the interaction of PD-1 with PD-L1 are desired.

### SUMMARY

The present disclosure provides macrocyclic compounds which inhibit the PD-1/PD-L1 protein/protein interaction, and are thus useful for the amelioration of various diseases, including cancer.

In certain aspects, the present disclosure provides a compound of formula (I):

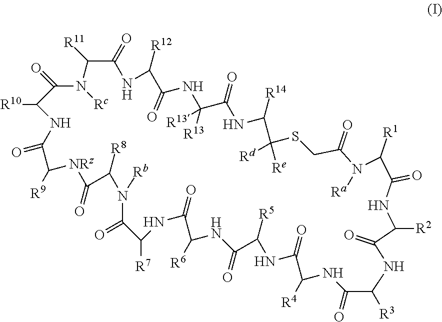

or a pharmaceutically acceptable salt thereof, wherein:
R¹ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₆alkyl, carboxyC₁-C₆alkyl, cyanoC₁-C₆alkyl, C₃-C₆cycloalkylcarbonylaminoC₁-C₆alkyl, guanidinylC₁-C₆alkyl, heteroarylC₁-C₆alkyl, heterocyclylC₁-C₆alkyl, hydroxyC₁-C₆alkyl, and methoxyC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, trifluoromethoxy, and trifluoromethyl, and wherein the heteroaryl part of the heteroarylC₁-C₆alkyl is optionally substituted with one, two, three, four, or five C₁-C₆alkyl groups;
R² is selected from arylC₁-C₆alkyl, guanidinylC₁-C₆alkyl, and heteroarylC₁-C₆alkyl; wherein the aryl part of the arylC₁-C₆alkyl is optionally substituted with one, two, three, four, or five groups independently selected from aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl; and wherein the heteroaryl part of the heteroarylC₁-C₆alkyl is optionally substituted with one, two, three, four, or five carboxyC₁-C₆alkyl groups;
R³ is carboxyC₁-C₆alkyl;
R⁴ is arylC₁-C₆alkyl or heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, cyano, halo, hydroxy, and trifluoromethyl; and wherein the heteroaryl part of the heteroarylC₁-C₆alkyl is optionally substituted with one, two, three, four, or five groups independently selected from C₁-C₆alkoxy, C₁-C₆alkyl, cyano, halo, hydroxy, and trifluoromethyl;
R⁵ is selected from C₂-C₆alkenyl, C₁-C₆alkyl, aminocarbonylC₁-C₆alkyl, aminocarbonylaminoC₁-C₆alkyl, C₅-C₆aryl, arylC₁-C₆alkyl, carboxyC₁-C₆alkyl, C₃-C₆cycloalkyl, and heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl;
R⁶ is biarylC₁-C₆alkyl; wherein the biaryl part of the biarylC₁-C₆alkyl is optionally substituted with one, two, three, four, or five groups independently selected from C₁-C₆alkoxy, C₁-C₆alkoxyC₁-C₆alkyl, C₁-C₆alkylcarbonylamino, aminocarbonyl, arylC₁-C₆alkoxy, cyanoC₁-C₆alkyl, halo, heteroaryl, trifluoromethoxy, and trifluoromethyl;
R⁷ is selected from C₁-C₆alkyl, C₁-C₆alkylaminoC₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₆alkyl, carboxyC₁-C₆alkyl, C₃-C₆cycloalkylcarbonylaminoC₁-C₆alkyl, guanidinylC₁-C₆alkyl, C₁-C₆haloalkylcarbonylaminoC₁-C₆alkyl, and hydroxyC₁-C₆alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl;
R⁸ is selected from C₁-C₆alkyl, aminocarbonylC₁-C₆alkyl, carboxyC₁-C₆alkyl, guanidinylC₁-C₆alkyl, and hydroxyC₁-C₆alkyl;
R^{z} is hydrogen and R⁹ is selected from C₁-C₆alkyl, aminoC₁-C₆alkyl, and C₃-C₆cycloalkylC₁-C₆alkyl; or
R⁹ and R^{z}, together with the atoms to which they are attached, form a proline ring;
R¹⁰ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, carboxyC₁-C₆alkyl, and heteroarylC₁-C₆alkyl;
R¹¹ is C₁-C₆alkyl or C₃-C₈cycloalkylC₁-C₆alkyl;
R¹² is selected from C₁-C₆alkyl, C₁-C₆alkylcarbonylaminoC₁-C₆alkyl, aminoC₁-C₆alkyl, haloC₁-C₆alkyl, heteroarylC₁-C₆alkyl, and hydroxyC₁-C₆alkyl;
R^{13′} is hydrogen and R¹³ is selected from C₁-C₆alkyl, C₁-C₆alkylcarbonylaminoC₁-C₆alkyl, C₂-C₆alkynyloxyC₁-C₆alkyl, aminoC₁-C₆alkyl, aminocarbonylC₁-C₆alkyl, aminocarbonylaminoC₁-C₆alkyl, arylC₁-C₆alkyl, azidoC₁-C₆alkyl, carboxyC₁-C₆alkyl, guanidinylC₁-C₆alkyl, hydroxyC₁-C₆alkyl, and heteroarylC₁-C₆alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl; and wherein the heteroaryl part of the heteroarylC₁-C₆alkyl is optionally substituted with one, two, three, four, or five groups independently selected from C₁-C₆alkyl, halo, haloarylcarbonylaminoC₁-C₆alkyl, and hydroxy; or
R¹³ and R^{13′}, together with the carbon atom to which they are attached, form a cyclopropyl ring;
R¹⁴ is —C(O)NR^{14′}CR¹⁵R^{15′}R^{15″}, —C(O)NH(CH₂)ⱼPh(CH₂)ⱼC(O)NHCHR¹⁷R^{17′}, —C(O)NH(CH₂)ⱼcyclopropyl(CH₂)ⱼC(O)NHCHR¹⁷R^{17′}, or —C(O)NR⁵⁰R⁵¹, wherein j is 0, 1, or 2, and wherein:
R⁵⁰ and R⁵¹, together with the nitrogen atom to which they are attached, form a piperazine ring, wherein the ring is further substituted with one —(CH₂)ⱼC(O)NHCHR¹⁷R^{17′} group;
R^{14′} is hydrogen or C₁-C₆alkyl, or R¹⁵ and R^{14′}, together with the atoms to which they are attached, form a morpholine, piperazine, or piperidine ring;
R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is —(CH₂)ₘCO₂H CH₂O((CH₂)₂O)ₙCH₂C(O)NHCHR¹⁶R¹⁶, or —C(O)NHCHR¹⁶R^{16′}; wherein:
R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{16′} is —(CH₂)ₘCO₂H, —CH₂O((CH₂)₂O)ₙCH₂C(O)NR⁷⁵CR^{17″}R¹⁷R^{17′},
-Ph(CH₂)ⱼC(O)NHCHR¹⁷R¹⁷ or —(CH₂)ⱼC(O)NHCHR¹⁷R^{17′}; wherein:
R⁷⁵ is hydrogen;
R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
R^{17″} is hydrogen, or R^{17″} and R¹⁷ form a C₃-C₈ cycloalkyl ring; wherein:
R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{18′} is —(CH₂)ₘCO₂H, —(CH₂)ₘC(O)NR¹⁹R^{19′}, or CH₂O((CH₂)₂O)ₙCH₂C(O)NHCHR¹⁹R^{19′}; wherein:
R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
R^{19′} is —(CH₂)ₘC(O)NR¹⁹R^{19′}, —(CH₂)ₘCO₂H, or —CH₂O((CH₂)₂O)ₙCH₂C(O)NHCHR²⁰R²⁰; wherein:
R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{20′} is —(CH₂)ₘCO₂H or —(CH₂)ₘC(O)NR²¹R^{21′}; wherein:
R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R²² is —(CH₂)ₘCO₂H or —(CH₂)ₘC(O)NR²²R^{22′}; wherein:
R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{22′} is —(CH₂)ₘCO₂H; wherein:
m is a integer from 1 to 10;
n is 1, 2, or 3; and
j is 0, 1, or 2;
R^{a} is hydrogen or C₁-C₆alkyl;
R^{b} is C₁-C₆alkyl, or aminoC₁-C₆alkyl;
R^{c} is hydrogen or C₁-C₆alkyl;
R^{d} is hydrogen or C₁-C₆alkyl; and
R^{e} is hydrogen or C₁-C₆alkyl.

In certain aspects, R¹ is selected from C₁-C₄alkyl, aminoC₁-C₄alkyl, aminocarbonylaminopropyl, aminocarbonylmethyl, arylC₁-C₂alkyl, tert-butylcarbonylaminoethyl, carboxyethyl, cyanoC₁-C₄alkyl, cyclopropylcarbonylaminoethyl, guanidinylC₃-C₄alkyl, heteroarylC₁-C₆alkyl, heterocyclylmethyl, hydroxyethyl, hydroxymethyl, methoxyethyl, and methoxymethyl; wherein the aryl part of the arylC₁-C₂alkyl is optionally substituted with one, two, or three groups independently selected from amino, aminoC₂-C₆alkoxy, aminoC₁-C₆alkyl, aminocarbonyl, carboxy, carboxymethoxy cyano, halo, hydroxy, methoxy, trifluoromethoxy, and trifluoromethyl.

In some aspects, R² is selected from aminoC₁-C₄alkyl, aminocarbonylmethyl, arylC₁-C₂alkyl, butyl, tert-butylcarbonylaminoC₂-C₄alkyl, guanidinylC₃-C₄alkyl, heteroarylC₁-C₆alkyl, hydroxymethyl, hydroxyethyl, and isopentyl; wherein the aryl part of the arylC₁-C₂alkyl is optionally substituted with one, two, or three groups independently selected from aminocarbonyl, carboxy, carboxymethoxy, carboxymethyl, cyano, fluoro, hydroxy, and trifluoromethyl.

In some aspects, R³ is carboxyC₁-C₄alkyl. In some aspects, R³ is carboxymethyl.

In some aspects, R⁴ is arylC₁-C₆alkyl or heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkyl, cyano, halo, and trifluoromethyl.

In some aspects, R⁴ is benzyl, optionally substituted with one, two, three, four, or five groups independently selected from C₁-C₆alkyl, cyano, halo, and trifluoromethyl.

In some aspects, R⁴ is indolylC₁-C₆alkyl.

In some aspects, R⁵ is selected from C₁-C₆alkyl, C₅-C₆aryl, arylC₁-C₆alkyl, carboxyC₂-C₃alkyl, C₃-C₆cycloalkyl, and heteroarylC₁-C₆alkyl; wherein the aryl part of the arylC₁-C₆alkyl is optionally substituted with one, two, three, four, or five groups independently selected from C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl.

In some aspects, R⁵ is arylmethyl or isopropyl; wherein the aryl part of the arylmethyl is optionally substituted with one, two, three, four, or five groups independently selected from aminocarbonyl, carboxy, carboxymethoxy, and hydroxy.

In some aspects, R⁵ is benzyl where the phenyl part is optionally substituted with one, two, three, four, or five groups selected from aminocarbonyl, carboxy, carboxymethoxy, and hydroxy.

In some aspects, R⁶ is biarylC₁-C₆alkyl; wherein the biaryl part of the biarylC₁-C₆alkyl is optionally substituted with one, two, or three fluoro groups.

In some aspects, R⁶ is biphenylC₁-C₆alkyl.

In some aspects, R⁷ is selected from C₁-C₆alkyl, C₁-C₆alkylcarbonylaminoC₁-C₆alkyl, aminocarbonylC₁-C₆alkyl, aminocarbonylaminoC₁-C₆alkyl, arylC₁-C₆alkyl, C₃-C₆cycloalkylcarbonylaminoC₁-C₆alkyl, guanidinylC₁-C₆alkyl, and hydroxyC₁-C₃alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl.

In some aspects, R⁷ is selected from aminocarbonylaminopropyl, aminocarbonylethyl, arylmethyl, isopentyl, isopropyl, and methylcarbonylaminobutyl; wherein the aryl part of the arylmethyl is optionally substituted with one, two, three, four, or five groups independently selected from carboxy and carboxymethoxy.

In some aspects, R⁸ is selected from of C₁-C₆alkyl, aminocarbonylC₁-C₆alkyl, carboxyC₁-C₆alkyl, guanidinylC₁-C₆alkyl, and hydroxymethyl.

In some aspects, R⁸ is methyl.

In some aspects, R⁹ is C₁-C₆alkyl or aminoC₁-C₆alkyl.

In some aspects, R⁹ is isobutyl.

In some aspects, R¹⁰ is aminoC₁-C₆alkyl or heteroarylC₁-C₆alkyl.

In some aspects, the heteroaryl in heteroarylC₁-C₆alkyl is imidazolyl.

In some aspects, R¹¹ is C₁-C₆alkyl or cyclohexylmethyl.

In some aspects, R¹² is selected from C₁-C₄alkyl, aminoC₁-C₆alkyl, and hydroxyC₁-C₆alkyl.

In some aspects, R¹³ is selected from aminobutyl, aminocarbonylethyl, aminoethyl, aminomethyl, carboxyethyl, hydroxyC₁-C₃alkyl imidazolylmethyl, methylcarbonylaminobutyl, and guanidinylpropyl.

In some aspects, the present disclosure provides a compound of formula (I), or a pharmaceutically acceptable salt thereof, wherein:
R¹ is selected from aminoC₁-C₄alkyl, aminocarbonylaminopropyl, aminocarbonylmethyl, arylC₁-C₂alkyl, butyl, tert-butylcarbonylaminoethyl, carboxyethyl, cyanomethyl, cyclopropycarbonylaminoethyl, ethyl, guanidinylC₃-C₄alkyl, hydroxyethyl, hydroxymethyl, isobutyl, methoxyethyl, methoxymethyl, methyl, heteroarylC₁-C₆alkyl, heterocyclylC₁-C₆alkyl, and propyl; wherein the aryl part of the arylC₁-C₂alkyl is optionally substituted with one, two, or three groups independently selected from amino, aminoC₂-C₆alkoxy, aminoC₁-C₆alkyl, aminocarbonyl, carboxy, carboxymethoxy, cyano, halo, hydroxy, methoxy, trifluoromethoxy, and trifluoromethyl;
R² is selected from arylC₁-C₂alkyl, guanidinylC₃-C₄alkyl, and heteroarylC₁-C₆alkyl; wherein the aryl part of the arylC₁-C₂alkyl is optionally substituted with one, two, or three groups independently selected from aminocarbonyl, carboxy, carboxyC₁-C₆alkoxy, cyano, hydroxy, trifluoromethoxy, and trifluoromethyl; wherein the heteroaryl part of the heteroarylC₁-C₆alkyl is optionally substituted with one, two, or three carboxymethoxy groups;
R³ is carboxymethyl;
R⁴ is arylmethyl or heteroarylmethyl; and wherein the aryl part of the arylmethyl is optionally substituted with one, two, three, four, or five substituents independently selected from chloro, cyano, fluoro, methyl, and trifluoromethyl;
R⁵ is selected from C₂-C₆alkenyl, C₁-C₆alkyl, aminocarbonylC₁-C₆alkyl, aminocarbonylaminoethyl, aminocarbonylaminopropyl, arylmethyl, carboxyethyl, carboxypropyl, C₃-C₆cycloalkyl, heteroarylC₁-C₆alkyl, and phenyl; wherein the aryl part of the arylmethyl is optionally substituted with one, two, three, four, or five groups independently selected from amino, aminocarbonyl, carboxy, carboxymethoxy chloro, cyano, fluoro, hydroxy, methoxy, methyl, and trifluoromethyl;
R⁶ is biarylC₁-C₆alkyl, wherein the biaryl part of the biarylC₁-C₆alkyl is optionally substituted with one, two, three, four, or five groups independently selected from chloro and fluoro;
R⁷ is selected from C₁-C₆alkyl, C₁-C₆alkylaminoC₁-C₆alkyl, aminoC₁-C₆alkyl, aminocarbonylaminopropyl, C₁-C₆alkylcarbonylaminoC₁-C₆alkyl, aminocarbonylethyl, arylmethyl, carboxyC₁-C₆alkyl, C₁-C₆cycloalkylcarbonylaminoC₁-C₆alkyl, guanidinylpropyl, C₁-C₆haloalkylcarbonylaminoC₁-C₆alkyl, hydroxymethyl, and methylcarbonylaminobutyl; wherein the aryl part of the arylmethyl is optionally substituted with one, two, three, four, or five groups independently selected from carboxy, carboxyC₁-C₆alkoxy, hydroxy, and trifluoromethyl;
R⁸ is selected from aminocarbonylmethyl, aminocarbonylethyl, carboxyethyl hydroxymethyl, and methyl;
R^{z} is hydrogen and R⁹ is selected from C₁-C₆alkyl, cyclopropylC₁-C₆alkyl, and cyclobutylmethyl; or
R⁹ and R^{z}, together with the atoms to which they are attached, form a proline ring;
R¹⁰ is selected from aminobutyl, aminoethyl, aminomethyl, aminopropyl, aminocarbonylmethyl, aminocarbonylaminopropyl, carboxymethyl, carboxyethyl, and imidazolylmethyl;
R¹¹ is isobutyl or cyclohexylmethyl;
R¹² is selected from C₁-C₄alkyl, C₁-C₆alkylcarbonylaminoC₁-C₆alkyl, aminoC₁-C₄alkyl, aminocarbonylaminopropyl, haloC₁-C₆alkyl, hydroxyC₁-C₄alkyl, and imidazolylmethyl;
R¹³ is selected from C₁-C₆alkylcarbonylaminoC₁-C₆alkyl, C₂-C₆alkynyloxyC₁-C₆alkyl aminoC₁-C₄alkyl, aminocarbonylC₁-C₃alkyl, aminocarbonylaminopropyl, azidoC₁-C₆alkyl, carboxyC₁-C₃alkoxy, carboxyC₁-C₃alkyl, hydroxyC₁-C₄alkyl, imidazolylmethyl, methylcarbonylaminobutyl, and triazolylmethyl optionally substituted with a haloarylcarbonylaminomethyl or guanidinylpropyl group;
R¹⁴ is —C(O)NR^{14′}CR¹⁵R^{15′}R^{15″}, wherein:
R^{14′} is hydrogen, C₁-C₆alkyl, or R¹⁵ and R^{14′}, together with the atoms to which they are attached, form a piperazine ring;
R¹⁵ is selected from hydrogen, C₁-C₁₆alkyl, aminoC₁-C₆alkyl, aminocarbonylC₁-C₆alkyl, carboxy, carboxyC₁-C₆alkyl, guanidinylC₁-C₆alkyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is hydrogen, carboxy, or —C(O)NHCHR¹⁶R^{16′}; wherein:
R¹⁶ is hydrogen, C₂-C₁₆alkyl, aminoC₁-C₆alkyl, aminocarbonylaminoC₁-C₆alkyl, or carboxyC₁-C₆alkyl;
and
R^{16′} is hydrogen, carboxy, —((CH₂)₂O)ₙCH₂C(O)NHCHR¹⁷R^{17′}, —(NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, aminoC₁-C₆alkyl, carboxy, or carboxyC₁-C₆alkyl; and
R^{17′} is —(CH₂)ₘC(O)NHR¹⁸R^{18′};
m is 0, 1, 2 or 3; wherein:
R¹⁸ is C₁₀-C₁₂alkyl; and
R^{18′} is carboxy;
R^{a} is hydrogen or methyl;
R^{b} is ethyl or methyl;
R^{c} is hydrogen;
R^{d} is hydrogen; and
R^{e} is hydrogen.

In some aspects, the present disclosure provides a compound of formula (I), or a pharmaceutically acceptable salt thereof, wherein
R¹ is selected from aminoC₁-C₄alkyl, aminocarbonylmethyl, butyl, tert-butylcarbonylaminoC₂-C₄alkyl, cyanomethyl, C₃-C₆cycloalkylcarbonylaminoC₁-C₆alkyl, guanidinylC₃-C₄alkyl, heteroarylC₁-C₆alkyl, heterocyclylC₁-C₆alkyl, hydroxyethyl, hydroxymethyl, isobutyl, methoxymethyl, and phenylC₁-C₂alkyl; wherein the phenyl part of the phenylC₁-C₂alkyl is optionally substituted with one, two, or three groups independently selected from aminocarbonyl, carboxy, carboxymethoxy cyano, fluoro, methoxy, and trifluoromethyl;
R² is selected from arylmethyl, guanidinylC₃-C₄alkyl, and heteroarylC₁-C₆alkyl; wherein the aryl part of the arylmethyl is optionally substituted with one, two, or three groups independently selected from aminocarbonyl, carboxy, carboxyC₁-C₆alkoxy, cyano, hydroxy, and trifluoromethyl;
R³ is carboxymethyl;
R⁴ is arylmethyl or heteroarylmethyl; wherein the aryl part of the arylmethyl is optionally substituted with one, two, three, four, or five groups independently selected from halo, methyl, and trifluoromethyl;
R⁵ is selected from arylmethyl, carboxyethyl, carboxypropyl, cyclohexyl, cyclopropyl, ethyl, heteroarylmethyl, isobutyl, isopropyl, and phenyl; wherein the aryl part of the arylmethyl is optionally substituted with one, two, three, four, or five groups independently selected from amino, aminocarbonyl, carboxy, carboxymethoxy fluoro, hydroxy, and trifluoromethyl;
R⁶ is biarylC₁-C₆alkyl, and wherein the biaryl part of the biarylC₁-C₆alkyl is optionally substituted with one, two, three, four, or five fluoro groups;
R⁷ is selected from aminocarbonylaminopropyl, aminocarbonylethyl, aminomethyl, arylmethyl, tert-butylcarbonylaminobutyl, carboxyethyl, C₃-C₆cycloalkylcarbonylaminoC₁-C₆alkyl, guanidinylpropyl, C₁-C₆haloalkylcarbonylaminoC₁-C₆alkyl, hydroxyethyl, isobutyl, isopropyl, and methylcarbonylaminobutyl; wherein the aryl part of the arylmethyl is optionally substituted with one, two, three, four, or five groups independently selected from carboxy, carboxyC₁-C₆alkyl, hydroxy, and trifluoromethyl;
R⁸ is methyl;
R⁹ is selected from cyclobutylmethyl, isobutyl, and methyl;
R¹⁰ is selected from aminocarbonylmethyl, aminoethyl, aminopropyl, carboxypropyl, and imidazolylmethyl;
R¹¹ is cyclohexylmethyl or isobutyl;
R¹² is selected from C₁-C₄alkyl, aminocarbonylaminopropyl, fluoroC₁-C₆alkyl, and hydroxyC₁-C₂alkyl;
R¹³ is selected from acetylaminobutyl, C₂-C₆alkynyloxymethyl, aminobutyl, aminocarbonylaminopropyl, aminocarbonylethyl, aminocarbonylmethyl, aminoethyl, aminomethyl, aminopropyl, carboxyethyl, carboxymethyl, carboxypropyl, ethyl, guanidinylpropyl, hydroxybutyl, hydroxyethyl, hydroxymethyl, imidazolylmethyl, and isopropyl;
R¹⁴ is —C(O)NR^{14′}CR¹⁵R^{15′}R^{15″}, wherein:
R^{14′} is hydrogen, C₁-C₆alkyl, or R¹⁵ and R^{14′}, together with the atoms to which they are attached, form a piperazine ring;
R¹⁵ is selected from hydrogen, methyl, C₁₀alkyl, aminomethyl, aminoethyl, aminopropyl, aminobutyl, carboxyethyl, aminocarbonylmethyl, hydroxymethyl, hydroxyethyl, guanidinylpropyl, and imidazolylmethyl;
R^{15′} is hydrogen or R¹⁵ and R^{15′}, together with the atoms to which they are attached, form a C₃-C₈cycloalkyl ring; and
R^{15″} is hydrogen, carboxy, or —C(O)NHCHR¹⁶R^{16′}; wherein:
R¹⁶ is hydrogen, C₂-C₁₆alkyl, aminomethyl, aminoethyl, aminocarbonylaminoC₁-C₆alkyl, or carboxyC₁-C₆alkyl; and
R^{16′} is hydrogen, C₁-C₆alkyl, carboxy, —((CH₂)₂O)ₙCH₂C(O)NHCHR¹⁷R^{17′}, (NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, carboxy, aminoethyl, carboxyC₁-C₆alkyl; and
R¹⁷ is —(CH₂)ₘC(O)NHR¹⁸R^{18′};
m is 0, 1, 2 or 3; wherein:
R¹⁸ is C₉-C₁₂alkyl; and
R^{18′} is carboxy;
R^{a} is hydrogen or methyl;
R^{b} is ethyl or methyl;
R^{c} is hydrogen;
R^{d} is hydrogen; and
R^{e} is hydrogen.

In some aspects, the present disclosure provides a compound of formula (I), or a pharmaceutically acceptable salt thereof, wherein:
R¹ is selected from aminoethyl, benzyl, butyl, guanidinylpropyl, hydroxyethyl, imidazolylC₁-C₂alkyl, morpholinylmethyl, and pyridinylC₁-C₂alkyl; wherein the phenyl part of the benzyl is optionally substituted with one, two, or three groups independently selected from aminocarbonyl, carboxy, carboxymethoxy, cyano, fluoro, and trifluoromethyl;
R² is benzyl or pyridinylC₁-C₆alkyl; wherein the phenyl part of the benzyl is optionally substituted with one, two, or three groups independently selected from carboxy and carboxyC₁-C₆alkoxy;
R³ is carboxymethyl;
R⁴ is benzyl or indolylmethyl; and wherein the phenyl part of the benzyl is optionally substituted with one or more groups independently selected from methyl and trifluoromethyl;
R⁵ is benzyl, isobutyl, or isopropyl, wherein the phenyl part of the benzyl is optionally substituted with one, two, three, four, or five groups independently selected from aminocarbonyl, carboxy, carboxymethoxy and hydroxy;
R⁶ is biphenylC₁-C₆alkyl;
R⁷ is selected from aminocarbonylaminopropyl, aminocarbonylethyl, benzyl, isopropyl, isobutyl, and methylcarbonylaminobutyl, wherein the phenyl part of the benzyl is optionally substituted with one, two, three, four, or five groups independently selected from carboxy and carboxymethoxy;
R⁸ is methyl;
R⁹ is isobutyl;
R¹⁰ is aminoethyl or imidazolylmethyl;
R¹¹ is cyclohexylmethyl;
R¹² is C₁-C₄alkyl or hydroxyC₁-C₂alkyl;
R¹³ is selected from aminobutyl, aminocarbonylethyl, aminoethyl, aminomethyl, carboxyethyl, carboxymethyl, guanidinylpropyl, hydroxyC₁-C₃alkyl imidazolylmethyl, and methylcarbonylaminobutyl;
R¹⁴ is —C(O)NR^{14′}CR¹⁵R^{15′}R^{15′}R^{15″}, wherein:
R^{14′} is hydrogen, C₁-C₆alkyl, or R¹⁵ and R^{14′}, together with the atoms to which they are attached, form a piperazinyl ring;
R¹⁵ is selected from hydrogen, methyl, C₁₀alkyl, and aminoethyl;
R^{15′} is hydrogen or R¹⁵ and R^{15′}, together with the atoms to which they are attached, form a cyclopropyl ring; and
R^{15″} is hydrogen, carboxy, or —C(O)NHCHR¹⁶R^{16′}; wherein:
R¹⁶ is hydrogen, C₂-C₁₆alkyl, aminoC₁-C₆alkyl, aminocarbonylaminoC₁-C₆alkyl, or carboxyC₁-C₆alkyl; and
R^{16′} is hydrogen, C₁-C₆alkyl, carboxy, —((CH₂)₂O)ₙCH₂C(O)NHCHR¹⁷R^{17′}, —(NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, carboxy, aminoC₁-C₆alkyl, carboxyC₁-C₆alkyl; and
R^{17′} is —(CH₂)ₘC(O)NHR¹⁸R¹⁸;
m is 0, 1, 2 or 3; wherein:
R¹⁸ is C₁₀alkyl; and
R^{18′}is carboxy;
R^{a} is hydrogen or methyl;
R^{b} is methyl;
R^{c} is hydrogen;
R^{d} is hydrogen; and
R^{e} is hydrogen.

In certain aspects, the present disclosure provides a compound of formula (Ia):

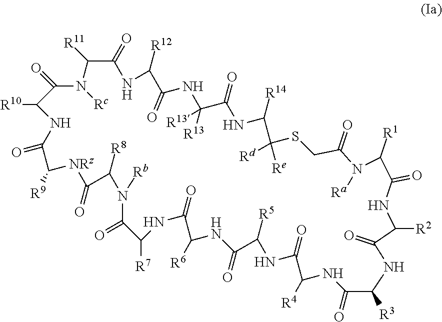

or a pharmaceutically acceptable salt thereof.

In certain aspects, the present disclosure provides a compound selected from the compounds listed in Table 3, or a pharmaceutically acceptable salt thereof.

In some aspects, the present disclosure provides a pharmaceutical composition comprising a compound of any of the preceding aspects, or a pharmaceutically acceptable salt thereof.

In some aspects, the present disclosure provides a method of enhancing, stimulating, and/or increasing an immune response in a subject in need thereof, wherein the method comprises administering to the subject a therapeutically effective amount of a compound of any one of the preceding aspects, or a pharmaceutically acceptable salt thereof.

In some aspects, the present disclosure provides a method of blocking the interaction of PD-1 with PD-L1 in a subject, wherein the method comprises administering to the subject a therapeutically effective amount of a compound of any one of the preceding aspects, or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of formula (I) or a pharmaceutically acceptable salt thereof” includes, but is not limited to, a compound of formula (I), two compounds of formula (I), a pharmaceutically acceptable salt of a compound of formula (I), a compound of formula (I) and one or more pharmaceutically acceptable salts of the compound of formula (I), and two or more pharmaceutically acceptable salts of a compound of formula (I).

The term “acetylamino,” as used herein, refers to —NHC(O)CH₃.

The term “acetylaminobutyl,” as used herein, refers to an acetylamino group attached to the parent molecular moiety through a butyl group.

The term “C₂-C₆alkenyl,” as used herein, refers to a group derived from a straight or branched chain hydrocarbon containing one or more carbon-carbon double bonds containing two to six carbon atoms.

The term “C₁-C₆alkoxy”, as used herein, refers to a C₁-C₆alkyl group attached to the parent molecular moiety through an oxygen atom.

The term “C₂-C₆alkoxy”, as used herein, refers to a C₂-C₆alkyl group attached to the parent molecular moiety through an oxygen atom.

The term “C₁-C₆alkoxyC₁-C₆alkyl”, as used herein, refers to a C₁-C₆alkoxy group attached to the parent molecular moiety through a C₁-C₆alkyl group.

The term “alkyl,” as used herein, refers to a group derived from a straight or branched chain saturated hydrocarbon containing carbon atoms. The term “alkyl” may be proceeded by “C_{#}-C_{#}” wherein the # is an integer and refers to the number of carbon atoms. For example, C₁-C₂alkyl contains one to two carbon atoms and C₁-C₃alkyl contains one to three carbon atoms.

The term “C₁-C₂alkylamino,” as used herein, refers to a group having the formula —NHR, wherein R is a C₁-C₂alkyl group.

The term “C₁-C₆alkylamino,” as used herein, refers to a group having the formula —NHR, wherein R is a C₁-C₆alkyl group.

The term “C₁-C₆alkylaminoC₁-C₆alkyl,” as used herein, refers to a C₁-C₆alkylamino group attached to the parent molecular moiety through a C₁-C₆alkyl group.

The term “C₁-C₆alkylcarbonyl,” as used herein, refers to a C₁-C₆alkyl group attached to the parent molecular moiety through a carbonyl group.

The term “C₁-C₂alkylcarbonylamino,” as used herein, refers to —NHC(O)R^{a}, wherein R^{a} is a C₁-C₆alkyl group.

The term “C₁-C₆alkylcarbonylamino,” as used herein, refers to —NHC(O)R^{a}, wherein R^{a} is a C₁-C₂alkyl group.

The term “C₁-C₂alkylcarbonylaminoC₁-C₆alkyl,” as used herein, refers to a C₁-C₂alkylcarbonylamino group attached to the parent molecular moiety through a C₁-C₆alkyl group.

The term “C₁-C₆alkylcarbonylaminoC₁-C₆alkyl,” as used herein, refers to a C₁-C₆alkylcarbonylamino group attached to the parent molecular moiety through a C₁-C₆alkyl group.

The term “C₂-C₆alkynyl,” as used herein, refers to a group derived from a straight or branched chain hydrocarbon containing one or more carbon-carbon triple bonds containing two to six carbon atoms.

The term “C₂-C₆alkynyloxy,” as used herein, refers to a C₂-C₆alkynyl group attached to the parent molecular moiety through an oxygen atom.

The term “C₂-C₆alkynyloxyC₁-C₆alkyl,” as used herein, refers to a C₂-C₆alkynyloxy group attached to the parent molecular moiety through a C₁-C₆alkyl group.

The term “C₂-C₆alkynyloxymethyl,” as used herein, refers to a C₂-C₆alkynyloxy group attached to the parent molecular moiety through a methyl group.

The term “amino,” as used herein, refers to —NH₂.

The term “aminoC₂-C₆alkoxy,” as used herein, refers to a C₂-C₆alkoxy group substituted with an amino group.

The term “aminoC₁-C₄alkyl,” as used herein, refers to an amino group attached to the parent molecular moiety through a C₁-C₄alkyl group.

The term “aminoC₁-C₆alkyl,” as used herein, refers to an amino group attached to the parent molecular moiety through a C₁-C₆alkyl group.

The term “aminobutyl,” as used herein, refers to a butyl group substituted with one or two amino groups.

The term “aminocarbonyl,” as used herein, refers to an amino group attached to the parent molecular moiety through a carbonyl group.

The term “aminocarbonylC₁-C₃alkyl,” as used herein, refers to an aminocarbonyl group attached to the parent molecular moiety through a C₁-C₃alkyl group.

The term “aminocarbonylC₁-C₆alkyl,” as used herein, refers to an aminocarbonyl group attached to the parent molecular moiety through a C₁-C₆alkyl group.

The term “aminocarbonylamino,” as used herein, refers to an aminocarbonyl group attached to the parent molecular moiety through an amino group.

The term “aminocarbonylaminoC₁-C₆alkyl,” as used herein, refers to an aminocarbonylamino group attached to the parent molecular moiety through a C₁-C₆alkyl group.

The term “aminocarbonylaminoethyl,” as used herein, refers to an aminocarbonylamino group attached to the parent molecular moiety through an ethyl group.

The term “aminocarbonylaminopropyl,” as used herein, refers to an aminocarbonylamino group attached to the parent molecular moiety through a propyl group.

The term “aminocarbonylethyl,” as used herein, refers to an aminocarbonyl group attached to the parent molecular moiety through a —CH₂CH₂ group.

The term “aminocarbonylmethyl,” as used herein, refers to an aminocarbonyl group attached to the parent molecular moiety through a —CH₂ group.

The term “aminoethyl,” as used herein, refers to —CH₂CH₂NH₂.

The term “aminomethyl,” as used herein, refers to —CH₂NH₂.

The term “aminopropyl,” as used herein, refers to a propoyl group substituted with one or two amino groups.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The term “aryl” may be proceeded by “C_{#}—C_{#}” wherein the # is an integer and refers to the number of carbon atoms in the aryl group. For example, C₅-C₆aryl contains five or six carbon atoms in the ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkoxy,” as used herein, refers to an arylC₁-C₆alkyl,” group attached to the parent molecular moiety through an oxygen atom.

The term “arylC₁-C₂alkyl,” as used herein, refers to an aryl group attached to the parent molecular moiety through a C₁-C₂alkyl group.

The term “arylC₁-C₆alkyl,” as used herein, refers to an aryl group attached to the parent molecular moiety through a C₁-C₆alkyl group.

The term “arylcarbonyl,” as used herein, refers to an aryl group attached to the parent molecular moiety through a carbonyl group.

The term “arylcarbonylamino,” as used herein, refers to —NR^{a}R^{b}, wherein R^{a} is hydrogen or methyl and R^{b} is an arylcarbonyl group.

The term “arylcarbonylaminoC₁-C₆alkyl,” as used herein, refers to an arylcarbonylamino group attached to the parent molecular moiety though a C₁-C₆alkyl group.

The term “arylmethyl,” as used herein, refers to an aryl group attached to the parent molecular moiety through a CH₂ group.

The term “azidoC₁-C₆alkyl,” as used herein, refers to an azido group attached to the parent molecular moiety through a C₁-C₆alkyl group.

The term “biaryl,” as used herein, refers to an aryl group substituted with one additional aryl group.

The term “biarylC₁-C₆alkyl,” as used herein, refers to a biaryl group attached to the parent molecular moiety through a C₁-C₆alkyl group.

The term “tert-butylcarbonylamino,” as used herein, refers to —NR^{a}R^{b} wherein R^{a} is hydrogen or methyl and R^{b} is a tert-butylcarbonyl group.

The term “tert-butylcarbonylaminoC₂-C₄alkyl,” as used herein, refers to a tert-butylcarbonylamino group attached to the parent molecular moiety through a C₂-C₄alkyl group.

The term “tertbutylcarbonylaminobutyl,” as used herein refers to-a tert-butylcarbonylamino group attached to the parent molecular moiety through a butyl group.

The term “tertbutylcarbonylaminoethyl,” as used herein refers to —CH₂CH₂NR^{a}R^{b}, wherein R^{a} is hydrogen or methyl and R^{b} is a tert-butylcarbonyl group.

The term “carbonyl,” as used herein, refers to —C(O)—.

The term “carboxy”, as used herein, refers to —CO₂H.

The term “carboxyC₁-C₃alkoxy,” as used herein, refers to a carboxyC₁-C₃alkyl group attached to the parent molecular moiety through an oxygen atom.

The term “carboxyC₁-C₆alkoxy,” as used herein, refers to a carboxyC₁-C₆alkyl group attached to the parent molecular moiety through an oxygen atom.

The term “carboxyC₁-C₃alkyl”, as used herein, refers to a carboxy group attached to the parent molecular moiety through a C₁-C₃alkyl group.

The term “carboxyC₁-C₆alkyl”, as used herein, refers to a carboxy group attached to the parent molecular moiety through a C₁-C₆alkyl group.

The term “carboxyC₂-C₃alkyl”, as used herein, refers to a carboxy group attached to the parent molecular moiety through a C₂-C₃alkyl group.

The term “carboxyethyl,” as used herein, refers to —CH₂CH₂CO₂H.

The term “carboxymethoxy,” as used herein, refers to —OCH₂CO₂H.

The term “carboxymethyl,” as used herein, refers to —CH₂CO₂H.

The term “carboxypropyl,” as used herein, refers to a propyl group substituted with one or two carboxy groups.

The term “cyano,” as used herein, refers to —CN.

The term “cyanoC₁-C₄alkyl,” as used herein, refers to a cyano group attached to the parent molecular moiety though a C₁-C₄alkyl.

The term “cyanoC₁-C₆alkyl,” as used herein, refers to a cyano group attached to the parent molecular moiety though a C₁-C₆alkyl.

The term “cyanomethyl,” as used herein, refers to —CH₂CN.

The term “C₃-C₆cycloalkyl”, as used herein, refers to a saturated monocyclic or bicyclic hydrocarbon ring system having three to six carbon atoms and zero heteroatoms. The bicyclic rings can be fused, spirocyclic, or bridged. Representative examples of cycloalkyl groups include, but are not limited to, cyclopropyl, cyclopentyl, and cyclohexyl.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, cyclohexyl, cycloheptyl, and cyclooctyl.

The term “(C₃-C₆cycloalkyl)C₁-C₆alkyl”, as used herein, refers to a C₃-C₆cycloalkyl group attached to the parent molecular moiety through a C₁-C₆alkyl group.

The term “C₃-C₆cycloalkylcarbonyl,” as used herein, refers to a C₃-C₆cycloalkyl group attached to the parent molecular moiety through a carbonyl group.

The term “C₃-C₆cycloalkylcarbonylamino,” as used herein, refers to a C₃-C₆cycloalkylcarbonyl group attached to the parent molecular moiety through an amino group.

The term “C₃-C₆cycloalkylcarbonylaminoC₁-C₆alkyl,” as used herein, refers to a C₃-C₆cycloalkylcarbonylamino group attached to the parent molecular moiety through a C₁-C₆alkyl group.

The term “cyclobutylmethyl,” as used herein, refers to a cyclobutyl group attached to the parent molecular moiety through a —CH₂ group.

The term “cyclohexylmethyl,” as used herein, refers to a cyclohexyl group attached to the parent molecular moiety through a —CH₂ group.

The term “cyclopropylC₁-C₆alkyl,” as used herein, refers to a cyclopropyl group attached to the parent molecular moiety through a C₁-C₆alkyl group.

The term “cyclopropylcarbonylaminoethyl,” as used herein, refers to —CH₂CH₂NHC(O)R, wherein R is a cyclopropyl group.

The term “fluoroC₁-C₆alkyl,” as used herein, refers to a C₁-C₆alkyl group substituted by one, two, three, or four fluoro groups.

The term “guanidinylC₁-C₆alkyl,” as used herein, refers to a NH₂C(NH)NH— group attached to the parent molecular moiety through a C₁-C₆alkyl group.

The term “guanidinylC₃-C₄alkyl,” as used herein, refers to a NH₂C(NH)NH— group attached to the parent molecular moiety through a C₃-C₄alkyl group.

The term “guanidinylpropyl,” as used herein, refers to a NH₂C(NH)NH— group attached to the parent molecular moiety through a propyl group.

The terms “halo” and “halogen”, as used herein, refer to F, Cl, Br, or I.

The term “C₁-C₆haloalkyl,” as used herein, refers to a C₁-C₆alkyl group substituted with one, two, three, or four halogen atoms.

The term “C₁-C₆haloalkylcarbonylamino,” as used herein, refers to —NR^{a}R^{b}, wherein R^{a} is hydrogen or methyl and R^{b} is a C₁-C₆haloalkylcarbonyl group.

The term “C₁-C₆haloalkylcarbonylaminoC₁-C₆alkyl,” as used herein, refers to a C₁-C₆haloalkylcarbonylamino group attached to the parent molecular moiety through a C₁-C₆alkyl group.

The term “haloarylcarbonylamino,” as used herein, refers to an arylcarbonylamino group substituted with one, two, three, four, or five halogen atoms.

The term “haloarylcarbonylaminoC₁-C₆alkyl,” as used herein, refers to an arylcarbonylaminoC₁-C₆alkyl group substituted with one, two, three, four, or five halogen atoms.

The term “haloarylcarbonylaminomethyl,” as used herein, refers to an haloarylcarbonylamino group attached to the parent molecular moiety through a —CH₂ group.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group.

The term “heteroarylmethyl,” as used herein, refers to a heteroaryl group attached to the parent molecular moiety through a CH₂ group.

The term “heterocyclyl,” as used herein, refers to a five-, six-, or seven-membered non-aromatic ring containing one, two, or three heteroatoms independently selected from nitrogen, oxygen, and sulfur. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through any substitutable atom in the group. Examples of heterocyclyl groups include, but are not limited to, morpholinyl, piperazinyl, pyrrolidinyl, and thiomorpholinyl.

The term “heterocyclylC₁-C₆alkyl,” as used herein, refers to a heterocyclyl attached to the parent molecular moiety through a C₁-C₆alkyl group.

The term “heterocyclylmethyl,” as used herein, refers to —CH₂R wherein R is a heterocyclyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₄alkyl,” as used herein, refers to a hydroxy group attached to the parent molecular moiety through a C₁-C₄alkyl group.

The term “hydroxyC₁-C₆alkyl,” as used herein, refers to a hydroxy group attached to the parent molecular moiety through a C₁-C₆alkyl group.

The term “hydroxybutyl,” as used herein, refers to a butyl group substituted with one or two hydroxy groups.

The term “hydroxyethyl,” as used herein, refers to —CH₂CH₂OH.

The term “hydroxymethyl,” as used herein, refers to —CH₂OH.

The term “imidazolylmethyl,” as used herein, refers to an imidazolyl group attached to the parent molecular moiety through a —CH₂ group.

The term “indolylC₁-C₆alkyl,” as used herein, refers to an indolyl group attached to the parent molecular moiety through a C₁-C₆alkyl group.

The term “indolylmethy,” as used herein, refers to an indolyl group attached to the parent molecular moiety through a —CH₂ group.

The term “methoxy,” as used herein, refers to —OCH₃.

The term “methoxyC₁-C₆alkyl,” as used herein, refers to a methoxy group attached to the parent molecular moiety though a C₁-C₆alkyl group.

The term “methoxyethyl,” as used herein, refers to —CH₂CH₂OCH₃.

The term “methoxymethyl,” as used herein, refers to —CH₂OCH₃.

The term “methylcarbonylamino,” as used herein, refers to —NHC(O)CH₃.

The term “methylcarbonylaminobutyl,” as used herein, refers to —(CH₂)₄NHC(O)CH₃.

The term “methylcarbonylaminobutyl,” as used herein, refers to —(CH₂)₃NHC(O)CH₃.

The term “morpholinylmethyl,” as used herein, refers to a morpholinyl group attached to the parent molecular moiety through a —CH₂ group.

The term “nitro,” as used herein, refers to —NO₂.

The term “phenylC₁-C₂alkyl,” as used herein, refers to a phenyl group attached to the parent molecular moiety through a C₁-C₂alkyl group.

The term “pyridinylC₁-C₂alkyl,” as used herein, refers to a pyridinyl group attached to the parent molecular moiety through a C₁-C₂alkyl group.

The term “pyridinylC₁-C₆alkyl,” as used herein, refers to a pyridinyl group attached to the parent molecular moiety through a C₁-C₆alkyl group.

The term “pyridinylmethyl,” as used herein, refers to a pyridinyl group attached to the parent molecular moiety through a —CH₂ group.

The term “triazolylmethyl,” as used herein, refers to a triazolyl group attached to the parent molecular moiety through a —CH₂ group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-L1”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PD1”, “hPD-1” and “hPD-I” are used interchangeably, and include variants, isoforms, species homologs of human PD-1, and analogs having at least one common epitope with PD-1. The complete PD-1 sequence can be found under GENBANK® Accession No. U64863.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

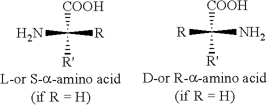

where R and R′ are as discussed herein. Unless otherwise indicated, the term “amino acid” as employed herein, alone or as part of another group, includes, without limitation, an amino group and a carboxyl group linked to the same carbon, referred to as “α” carbon, where R and/or R′ can be a natural or an un-natural side chain, including hydrogen. The absolute “S” configuration at the “α” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocyclic peptides described herein, the dosage ranges from about 0.0001 to 100 mg/kg, and more usually 0.01 to 40 mg/kg, of the host body weight. For example dosages can be 0.3 mg/kg body weight, 1 mg/kg body weight, 3 mg/kg body weight, 5 mg/kg body weight, 10 mg/kg body weight, 20 mg/kg body weight, 30 mg/kg body weight, 40 mg/kg body weight, or within the range of 10-40 mg/kg. An exemplary treatment regime entails administration once per day, bi-weekly, tri-weekly, weekly, once every two weeks, once every three weeks, once every four weeks, once a month, once every 3 months or once every three to 6 months. Preferred dosage regimens for a macrocyclic peptide of the disclosure include 1 mg/kg body weight or 3 mg/kg body weight via intravenous administration, with the macrocyclic peptide being given using one of the following dosing schedules: (i) every four weeks for six dosages, then every three months; (ii) every three weeks; (iii) 3 mg/kg body weight once followed by 1 mg/kg body weight every three weeks.

In another aspect, the disclosure pertains to methods of inhibiting growth of tumor cells in a subject using the macrocyclic compounds of the present disclosure. In certain embodiments, the compounds of the present disclosure are capable of binding to PD-1, disrupting the interaction between PD-1 and PD-L1, competing with the binding of PD-1 with certain anti-PD-1 monoclonal antibodies that are known to block the interaction with PD-L1, and enhancing CMV-specific T cell IFNγ secretion. As a result, the compounds of the present disclosure can be useful for modifying an immune response, treating diseases such as cancer, stimulating a protective autoimmune response, or to stimulate antigen-specific immune responses (e.g., by co-administration of PD-L1 blocking compounds with an antigen of interest). For example, the compounds of the present disclosure can be used to treat cancers selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

Compounds of the present disclosure can also be used in treating infectious diseases, such as those caused by a virus. Examples of such viruses include, but are not limited to, HIV, Hepatitis A, Hepatitis B, Hepatitis C, herpes viruses, and influenza.

Compounds of the present disclosure can also be used in treating septic shock.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of formula (I) and/or at least one salt thereof with at least one inert solid diluent, such as, for example, calcium carbonate; calcium phosphate; and kaolin.

Soft gelatin capsules can, for example, be prepared by mixing at least one compound of formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of formula (I) and/or at least one pharmaceutically acceptable salt thereof can, for example, be prepared as an oil-in-water emulsion. The oily phase of the emulsions comprising the compounds of formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least one emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

In certain embodiments, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some embodiments, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### EXAMPLES

The following Examples are included to demonstrate various aspects of the present disclosure. It should be appreciated by those of skill in the art that the techniques disclosed in the Examples that follow represent techniques discovered by the inventors to function well in the practice of the disclosure, and thus can be considered to constitute preferred modes for its practice. However, those of skill in the art should, in light of the present disclosure, appreciate that many changes can be made in the specific Compounds which are disclosed and still obtain a like or similar result without departing from the spirit and scope of the disclosure.

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and Compounds are defined as follows: Ph=phenyl; Bn=benzyl; i-Bu=iso-butyl; i-Pr=iso-propyl; Me=methyl; Et=ethyl; Pr=n-propyl; Bu=n-butyl; t-Bu=tert-butyl; Trt=trityl; TMS=trimethylsilyl; TIS=triisopropylsilane; Et₂O=diethyl ether; HOAc or AcOH=acetic acid; MeCN or AcCN=acetonitrile; DMF=N,N-dimethylformamide; EtOAc=ethyl acetate; THF=tetrahydrofuran; TFA=trifluoroacetic acid; TFE=α,α,α-trifluoroethanol; Et₂NH=diethylamine; NMM=N-methylmorpholine; NMP=N-methylpyrrolidone; DCM=dichloromethane; TEA=trimethylamine; min.=minute(s); h or hr=hour(s); L=liter; mL or ml=milliliter; L=microliter; g=gram(s); mg=milligram(s); mol=mole(s); mmol=millimole(s); meq=milliequivalent; rt or RT=room temperature; sat or sat'd=saturated; aq.=aqueous; mp=melting point; BOP reagent=benzotriazol-1-yloxy-tris-dimethylamino-phosphonium hexafluorophosphate (Castro's reagent); PyBOP reagent=benzotriazol-1-yloxy-tripyrrolidino phosphonium hexafluorophosphate; HBTU=2-(1H-Benzotriazol-1-yl)-1,1,3,3-tetramethyluronim hexafluorophosphate; HATU=O-(7-Azabenzotriazol-1-yl)-1,1,3,3-tetramethyluronim hexafluorophosphate; HCTU=2-(6-Chloro-1-H-benzotriazol-1-yl)-1,1,3,3-tetramethyluronium hexafluorophosphate; T3P=2,4,6-tripropyl-1,3,5,2,4,6-trioxatriphosphorinane-2,4,6-trioxide; DMAP=4-(dimethylamino)pyridine; DIEA=diisopropylethylamine; Fmoc or FMOC=fluorenylmethyloxycarbonyl; Boc or BOC=tert-butyloxycarbonyl; HOBT or HOBT·H₂O=1-hydroxybenzotriazole hydrate; Cl-HOBt=6-Chloro-benzotriazole; HOAT=1-hydroxy-7-azabenzotriazole; HPLC=high performance liquid chromatography; LC/MS=high performance liquid chromatography/mass spectrometry; MS or Mass Spec=mass spectrometry; NMR=nuclear magnetic resonance; Sc or SC or SQ=sub-cutaneous; and IP or ip=intra-peritoneal.

### Example 1: General Synthetic Procedures and Analytical Methods

### Peptide Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-C1-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown below.
Examples of Orthogonally Protected Amino Acids used in Solid Phase Synthesis

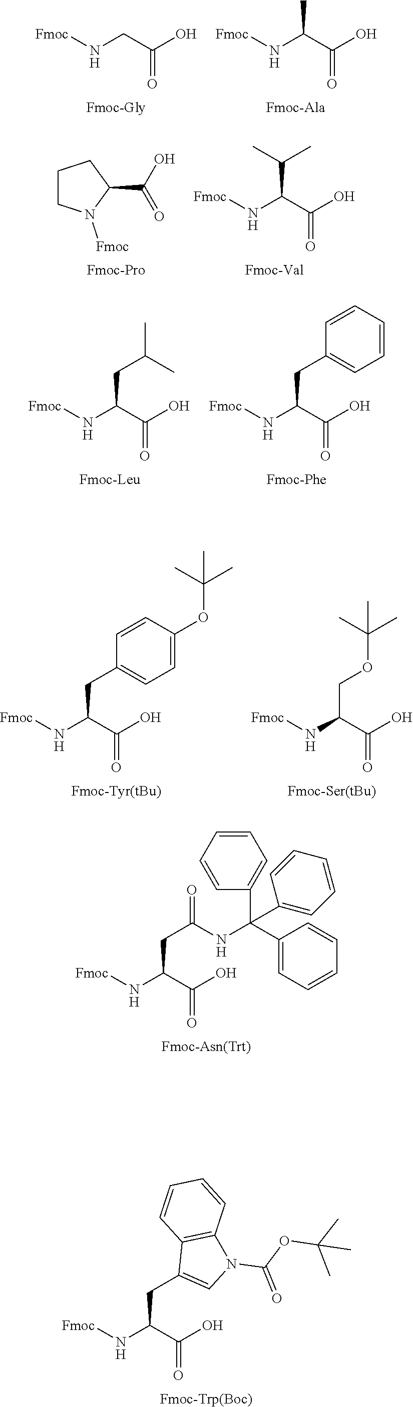

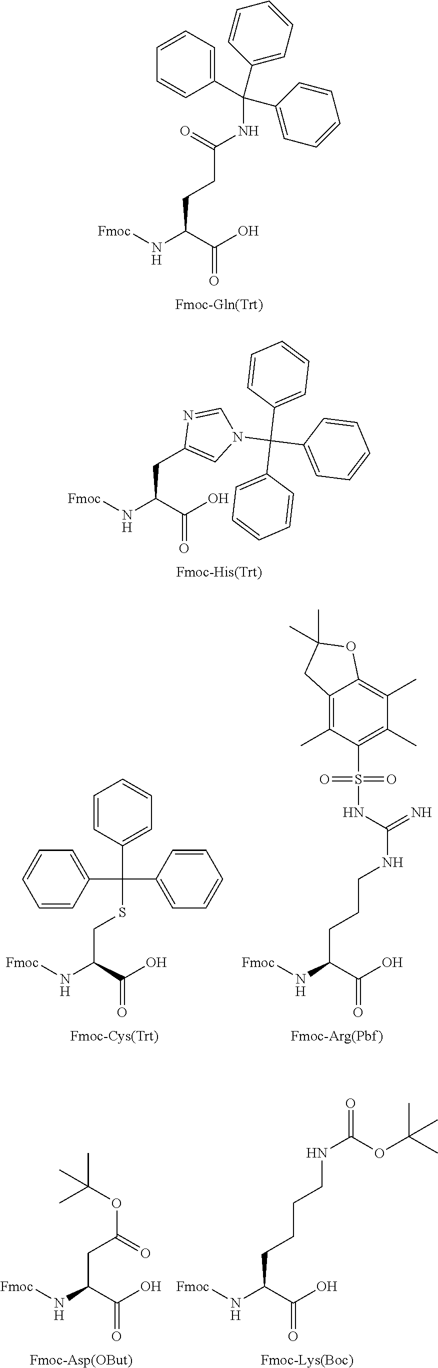

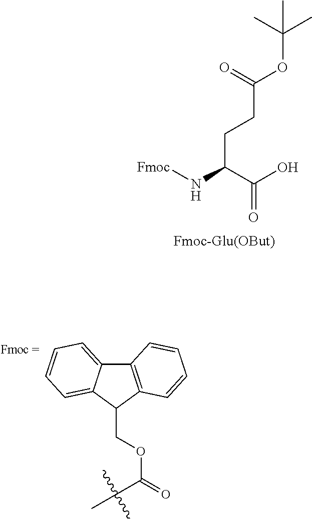

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Peptides with the desired purity can be obtained by purification using preparative HPLC, for example, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

List of unnatural amino acids referred to herein is provided below:

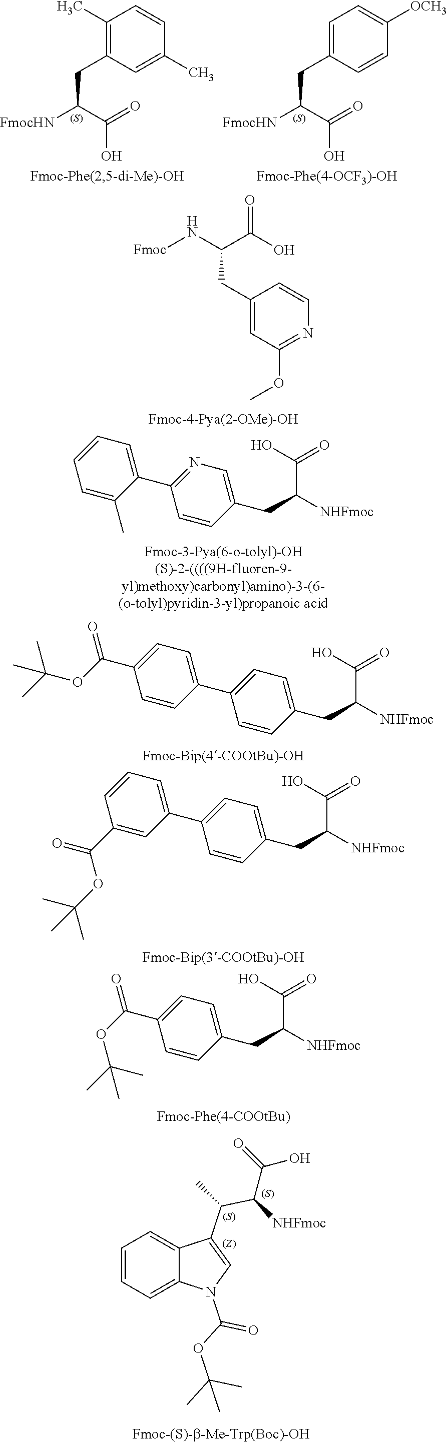

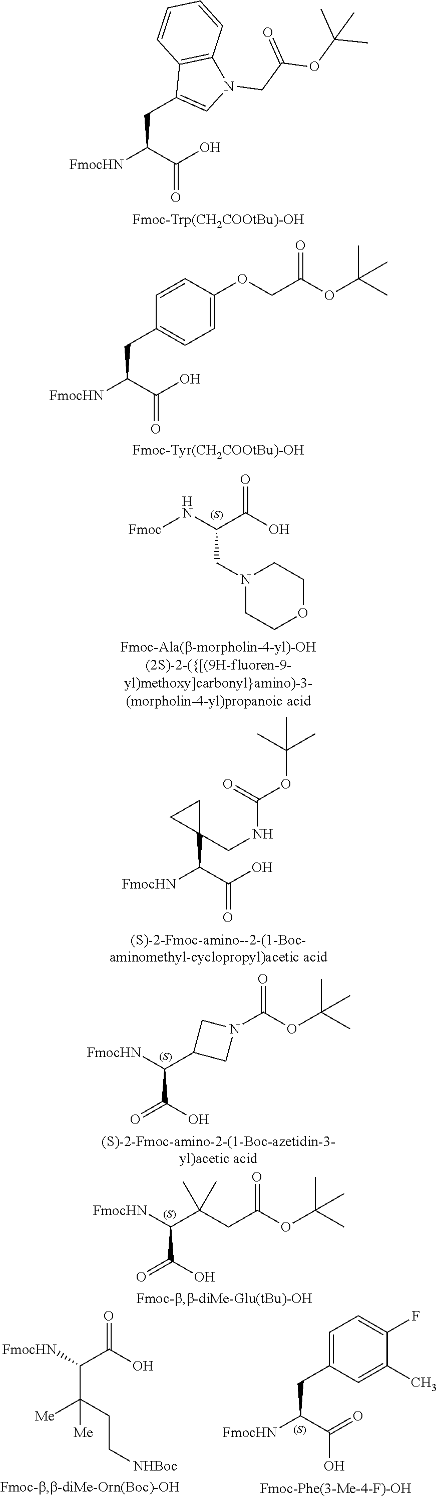

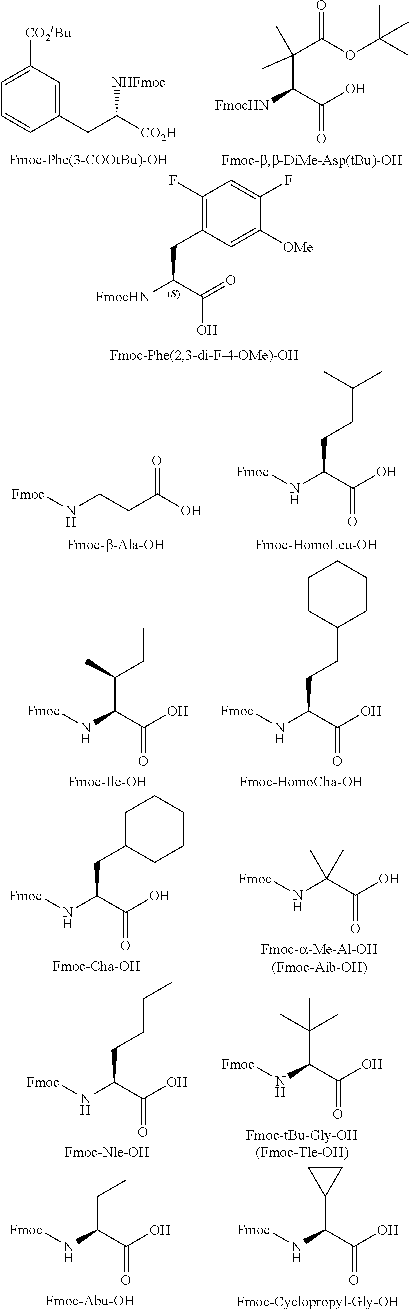

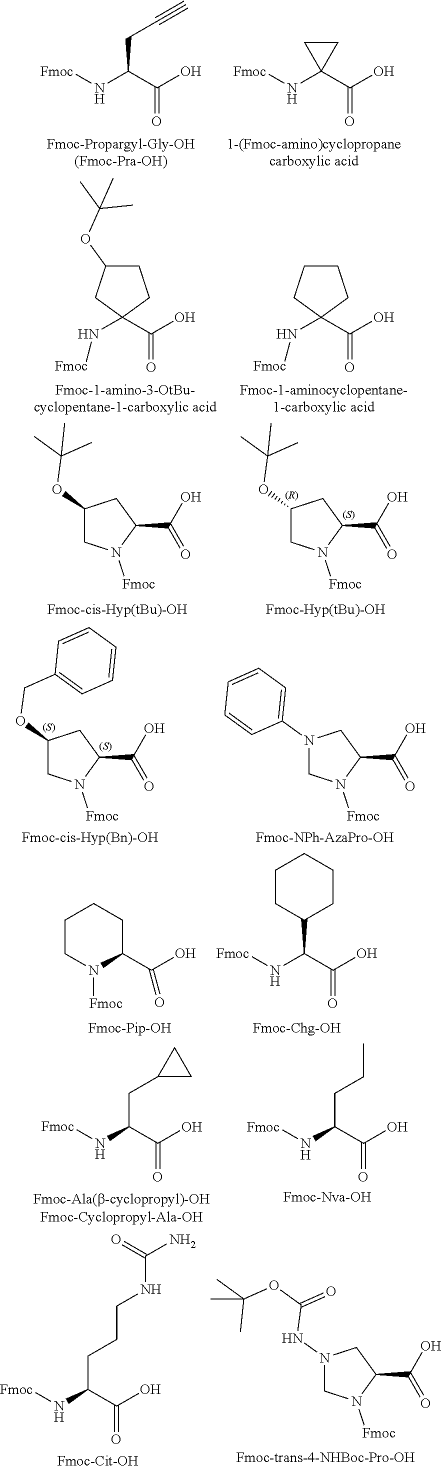

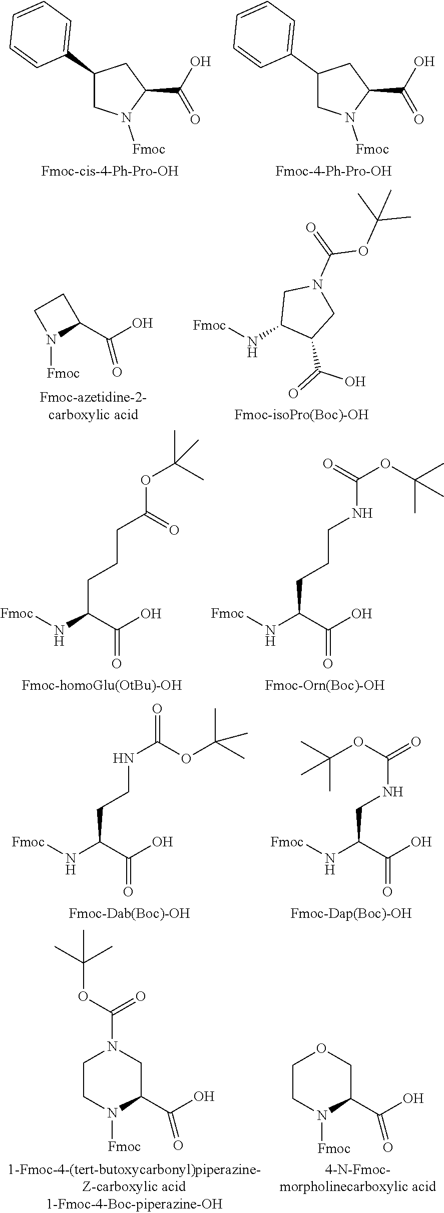

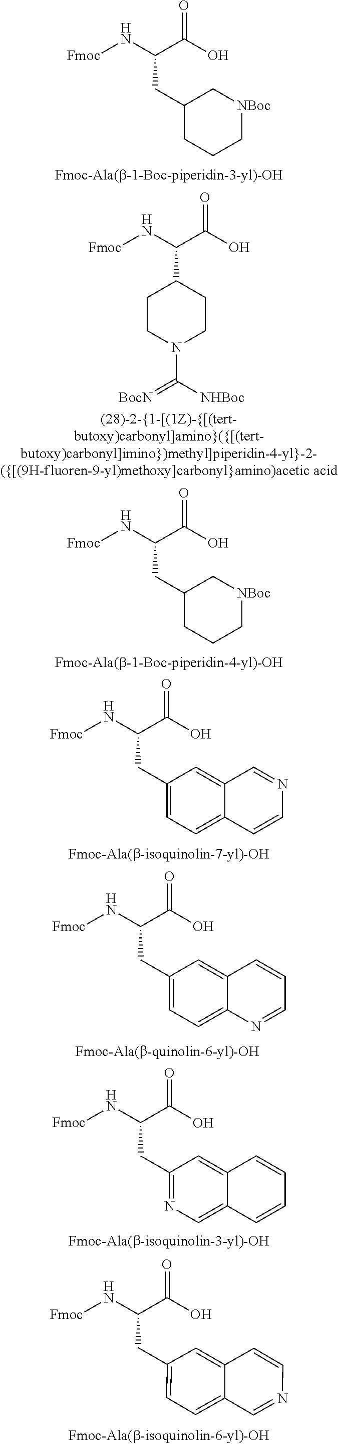

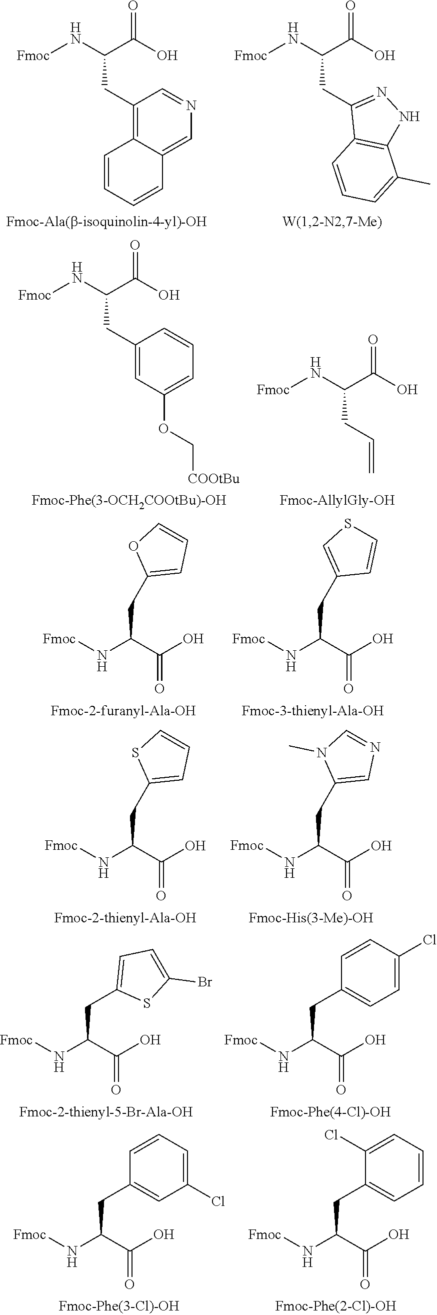

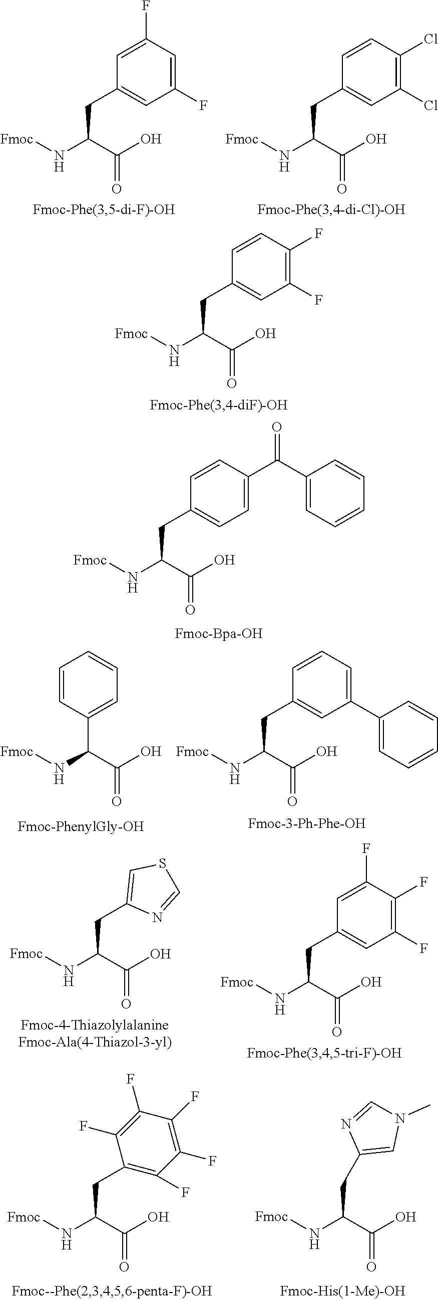

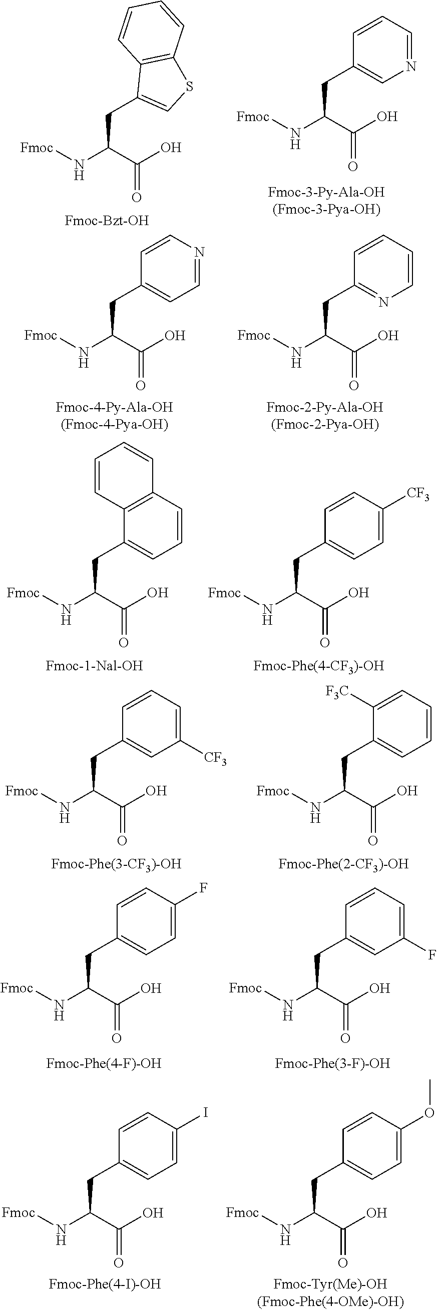

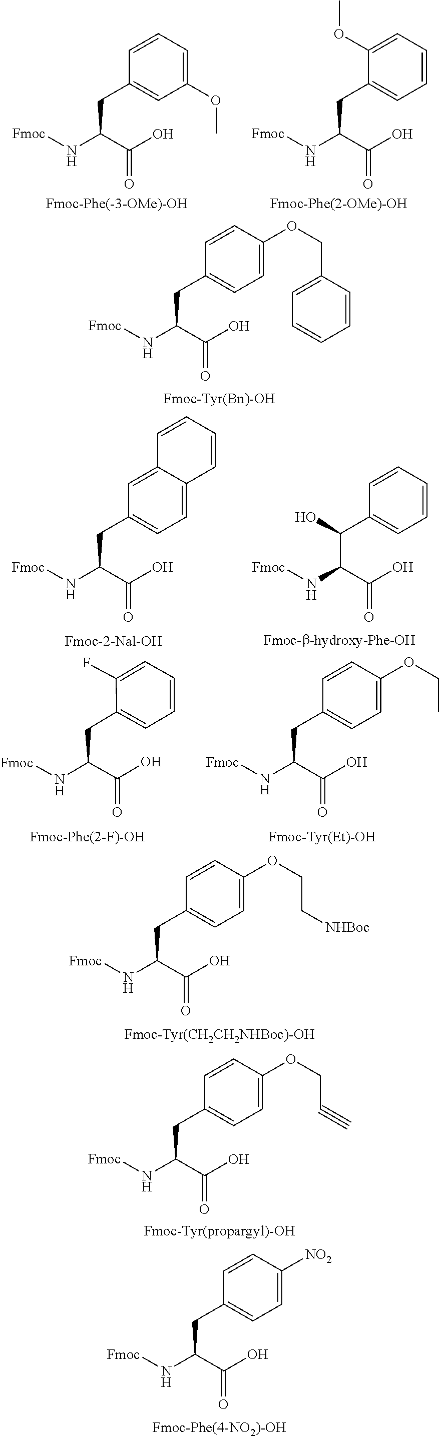

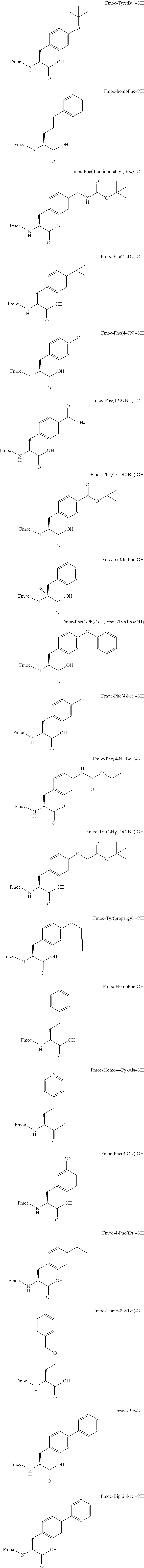

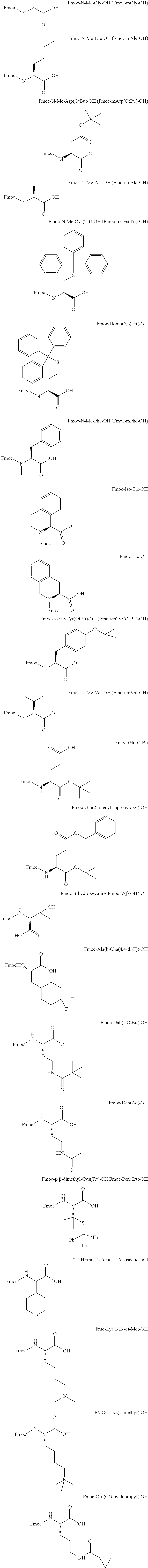

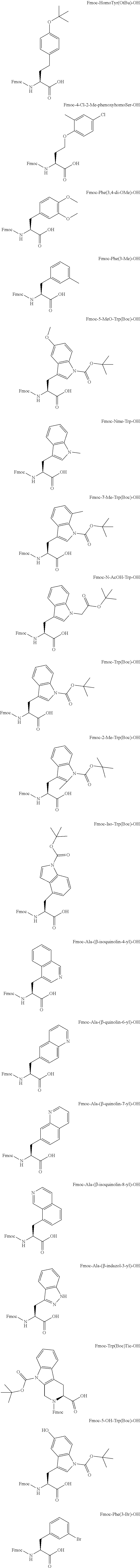

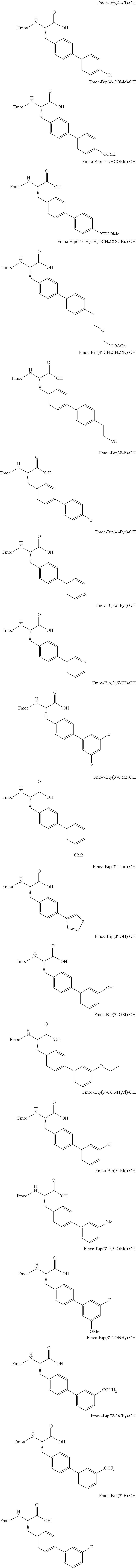

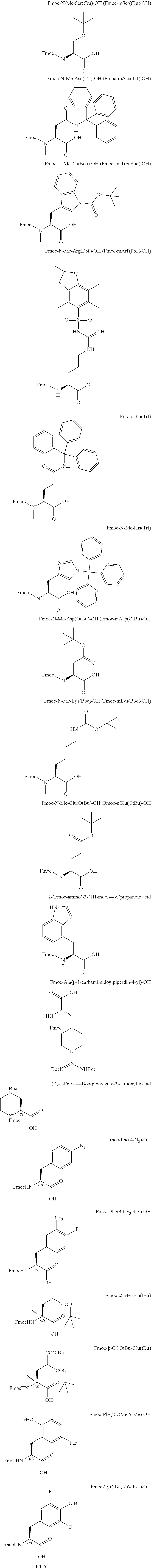

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m/z” unit designation. Compounds with exact masses greater than 1000 were often detected as double-charged or triple-charged ions. The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LC/MS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LC/MS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LC/MS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LC/MS Condition F:

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LC/MS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LC/MS Condition H.

Column: X Bridge C18, 4.6×50 mm, 5-μm particles; Mobile Phase A: 10 mM NH₄₀Ac; Mobile Phase B: methanol, Temperature: 35° C.; Gradient: 5-95% B over 4 minutes; Flow: 4.0 mL/min; Detection: UV at 220 nm.

### Analytical LC/MS Condition I:

Column: X Bridge C18, 4.6×50 mm, 5-μm particles; Mobile Phase A: 10 mM NH₄₀Ac; Mobile Phase B: acetonitrile, Temperature: 35° C.; Gradient: 5-95% B over 4 minutes; Flow: 4.0 mL/min; Detection: UV at 220 nm.

### Analytical LC/MS Condition J:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LC/MS Condition K:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 100% B; Flow: 0.80 mL/min; Detection: UV at 220 nm.

### Analytical LC/MS Condition L:

Column: Waters Acquity UPLC BEH C18, 2.1×50 mm, 1.7-μm particles; Buffer: 10 mM Ammonium Acetate. Mobile Phase A: buffer” CH₃CN (95/5); Mobile Phase B: Mobile Phase B:Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LC/MS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### General Procedures:

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis. Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel remained attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below.

### Resin-Swelling Procedure:

To a 25-mL polypropylene solid-phase reaction vessel was added Sieber resin (70 mg, 0.05 mmol). The resin was washed (swelled) as follows: to the reaction vessel was added DMF (2.0 mL), upon which the mixture was periodically agitated for 10 minutes before the solvent was drained through the frit.

### Single-Coupling Procedure:

To the reaction vessel containing the resin from the previous step was added DMF (2.5 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). The mixture was periodically agitated for 5.0 minutes and then the solution was drained through the frit. The resin was washed successively six times as follows: for each wash, DMF (2.5 mL) was added to the vessel and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 equiv). The mixture was periodically agitated for 30-120 minutes, then the reaction solution was drained through the frit. The resin was washed successively six times as follows: for each wash, DMF (2.5 mL) was added and the resulting mixture was periodically agitated for 30 seconds before the solution was drained through the frit. The resulting resin was used directly in the next step.

### Single-Coupling Pre-Activation Procedure:

To the reaction vessel containing the resin from the previous step was added DMF (3.75 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). To the resin was added piperidine:DMF (20:80 v/v, 3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the premixed amino acid and HATU (0.1 M in DMF, 1.25 mL, 1:1 ratio 2.5 equiv), then NMM (0.8 M in DMF, 1.25 mL, 20 equiv). The mixture was periodically agitated for 2-3 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5 mL) three times, upon which the mixture was agitated for 30 seconds before the solvent was drained through the frit each time. To the reaction vessel was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 equiv). The mixture was periodically agitated for 1 hour, then the reaction solution was drained through the frit. The resin was washed twice with DMF (3.75 mL) and the resulting mixture was periodically agitated for 30 seconds before the solution was drained through the frit each time. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 eq). The mixture was periodically agitated for 1-2 hours, then the reaction solution was drained through the frit. The resin was successively washed six times as follows: for each wash, DMF (3.75 mL) was added and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.75 mL) three times, upon which the mixture was agitated for 30 seconds before the solvent was drained through the frit each time. To the reaction vessel containing the resin from the previous step was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis:

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Global Deprotection Method A:

poUnless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml bio-rad poly-prep chromatography column was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1, v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

### N-Methylation On-Resin Method A.

To the resin (50 μmol) in a Bio-Rad tube was added CH₂Cl₂ (2 mL) and shaken for 5 min at rt. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at rt for 2 h. The solvent was drained and the resin was rinsed with CH₂Cl₂ (5 mL×3), DMF (5 mL×3) and then THF (5 mL×3). The resin was added THF (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 1-16 h. The solvent was drained, and the resin was washed with THF (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×). Air dried and used directly in the next step.

N-Methylation On-Resin Method B (Turner, R. A. et al, Org. Lett., 15(19):5012-5015 (2013)).

All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0. 119 ml, 1.00 mmol), 1,8-diazabicyclo[5.4.0]undec-7-ene (0.181 ml, 1.20 mmol). The mixture was placed on a shaker for 60 min. The solution was drained through the frit. The resin was washed successively three times with DMF (4.0 mL) and three times with DCM (4.0 mL). The resin was washed three times with dry THF (2.0 mL) to remove any residual water. In an oven-dried 4.0 mL vial was added THF (1.0 mL) and triphenylphosphine (131 mg, 0.500 mmol) on dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed with three times with dry THF (2.0 mL) to remove any residual water. In an oven-dried 4.0 mL vial was added THF (1.0 mL), triphenylphosphine (131 mg, 0.50 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THF (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THE was added to nosylated resin (0.186 g, 0.100 mmol), and the reaction mixture was stirred for 16 hours at room temperature. The resin was washed three times with THF (5 mL) Tetrahydrofuran, and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMP (3 mL). A solution of NMP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preloading Amines on the PL-FMP Resin:

PL-FMP resin (Novabiochem, 1.00 mmol/g substitution) was swollen with DMF (20 mL/mmol) at room temperature. The solvent was drained and 10 ml of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed by DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and got weight to calculate resin loading.

### General Procedure for Preloading (Fmoc Amino) Acids on Cl-Trityl Resin:

To a glass reaction vessel equipped with a frit was added the 2-Chloro-chlorotrityl resin mesh 50-150, (1.54 meq/gram, 1.94 grams, 3.0 mmole) to be swollen in DCM (5 mL) for 5 minutes. A solution of the acid (3.00 mmol, 1.0 eq) in DCM (5 mL) was added to the resin followed by DIPEA (2.61 ml, 15.00 mmol, 5.0 eq). The reaction was shaken at room temperature for 60 minutes. Add in DIEA (0.5 mL) and methanol (3 mL), shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen. The resin loading can be determined as follows:

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm and zero with the blank solution followed by the reading of the solution, Absorbance=1.9411 (1.9411/20 mg)*6.94=0.6736. Loading of the resin was measured to be 0.6736 mmol/g.

### Click Reaction On-Resin Procedure Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2×5 mL×5 mins). In a 200-ml bottle was charged with 30 time of the following: vitamin C (0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THF (1.5 ml), followed by DIPEA (0.087 ml, 0.50 mmol) and the azide, tert-butyl (S)-1-azido-40-(tert-butoxycarbonyl)-37,42-dioxo-3,6,9,12,15,18,21,24,27,30,33-undecaoxa-36,41-diazanonapentacontan-59-oate (0.028 g, 0.025 mmol). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Procedure Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 uL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 uL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube is placed 50 umoles of dried Rink resin of a N-terminus Fmoc-protected linear polypeptide containing 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1-bis(di-tert-butylphosphino)ferrocene]dichloropalladium(II) [PdCl₂(dtbpf)] (3.26 mg, 5.00 μmol). The tube was shaken at rt overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 ml TFA at rt for 1 h. The rest of the resin was used in the next step of peptide coupling or chloroacetic acid capping of the N-terminus.

### Preparation of (S)-2-((((9H-fluoren-9-yl)methoxy)carbonyl)-3-(1-(2-(tert-butoxy)-2-oxoethyl)-1H-indol-3-yl)propanoic Acid

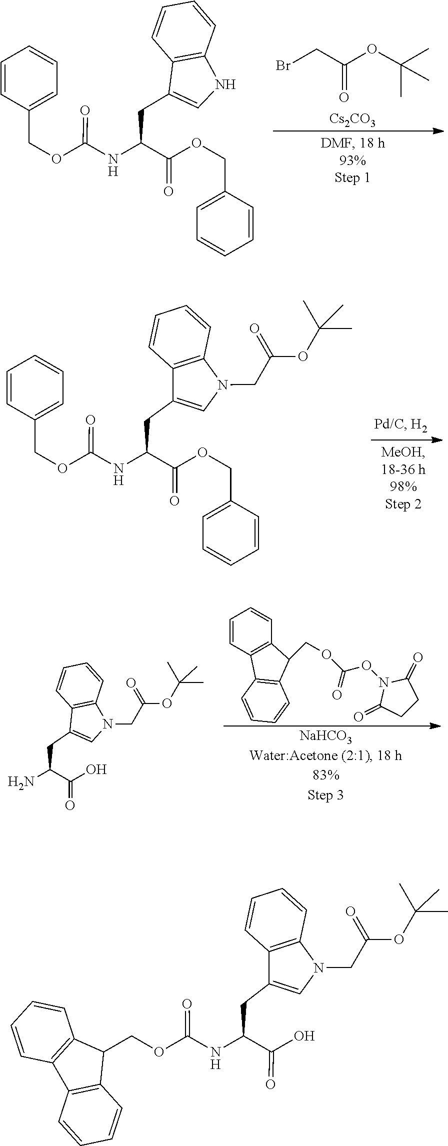

### Step 1:

To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

### Step 2:

H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd—C (1.45 g, 1.36 mmol) in MeOH (200 mL) at RT for 10 min. The mixture was then stirred under positive pressure of H2 while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

### Step 3:

To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic Acid

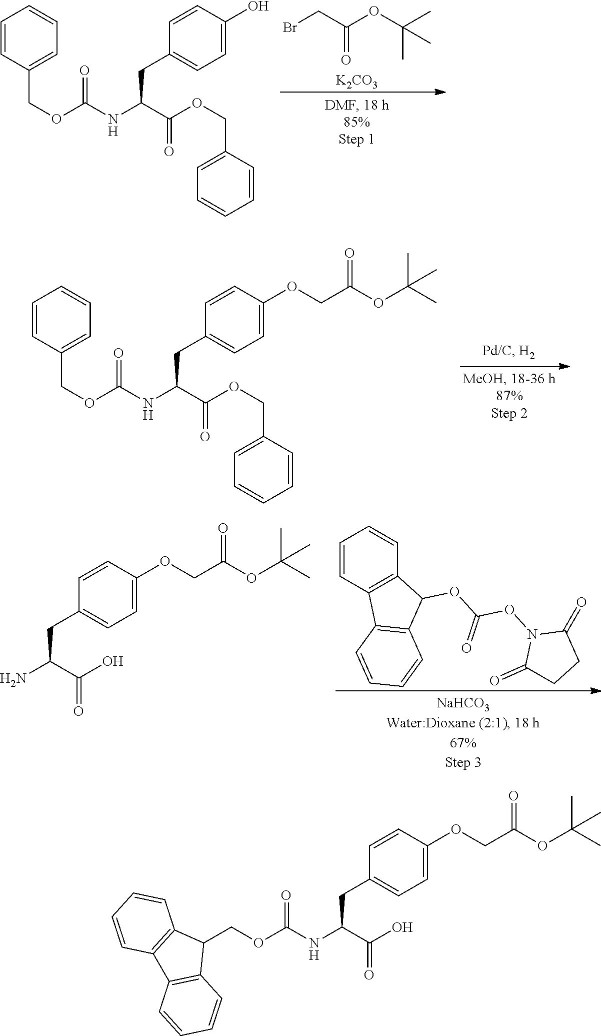

### Step 1:

To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford colorless gum. The crude compound was purified by flash column chromatography using 20% ethyl acetate in petroleum ether as an eluent to afford a white solid (78 g, 85%).

### Step 2:

The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite®) and washed with methanol (1000 mL). The filtrate was concentrated under vacuum to afford a white solid (36 g, 87%).

### Step 3:

To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of (S)-2-((((9H-fluoren-9-yl)methoxy)carbonyl)amino)-3-(4-(tert-butoxycarbonyl)phenyl)propanoic Acid

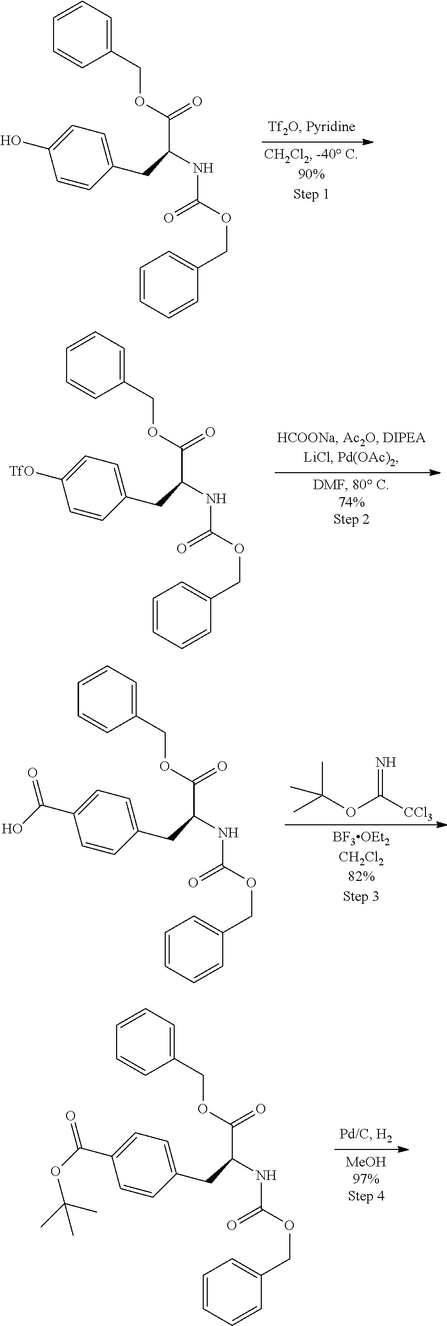

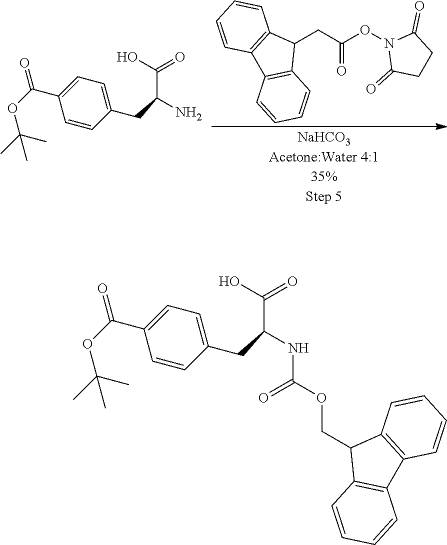 [SMILES: CC(C)(C)OC(=O)c1ccc(C[C@H](N)C(=O)O)cc1 ; O=C(CC1c2ccccc2-c2ccccc21)ON1C(=O)CCC1=O ; CC(C)(C)OC(=O)c1ccc(C[C@H](NC(=O)OCC2c3ccccc3-c3ccccc32)C(=O)O)cc1]

### Step 1:

(S)-Benzyl 2-(((benzyloxy)carbonyl)amino)-3-(4-hydroxyphenyl)propanoate (10 g, 24.66 mmol) was taken in DCM (100 mL) in a 250 mL multi-neck round bottom flask under magnetic stirring with N₂ outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at −40° C. for 2 hours. The reaction mixture was quenched with water at −10° C., and then added citric acid solution (50 mL). The organic layer was extracted in DCM, and the separated organic layer was dried over anhydrous Na₂SO₄, filtered, and then evaporated to give (S)-benzyl 2-(((benzyloxy)carbonyl)amino)-3-(4-(((trifluoromethyl)sulfonyl)oxy)phenyl)propanoate (11.93 g, 22.20 mmol, 90% yield) as a pale yellow solid.

### Step 2:

A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy)carbonyl)amino)-3-(4-(((trifluoromethyl)sulfonyl)oxy)phenyl)propanoate (300 g, 558 mmol), lithium chloride (71 g, 1675 mmol), 1,3-bis(diphenylphosphino)propane (24.17 g, 58.6 mmol) were added followed by the addition of palladium(II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%).

### Step 3:

To a 2000-ml multi-neck round-bottomed flask was charged (S)-4-(3-(benzyloxy)-2-(((benzyloxy)carbonyl)amino)-3-oxopropyl)benzoic acid (130 g, 300 mmol), dichloromethane (260 mL) and cyclohexane (130 mL). To the slurry reaction mixture was added BF₃·OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using 1.5 Kg silicycle column. The product spot was eluted at 15% ethyl acetate/petroleum ether mixture. The collected fractions were concentrated to obtain a colorless liquid (120 g, yield 82%).

### Step 4:

(S)-tert-Butyl 4-(3-(benzyloxy)-2-(((benzyloxy)carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H2 for 16 h at room temperature. The reaction mass was filtered through diatomaceous earth (Celite®) and the bed was washed with methanol The obtained filtrate was concentrated to obtain a pale yellow solid. The obtained solid was stirred with 5% methanol:diethyl ether mixture for 15 min before being filtered, dried under vacuum to obtain a pale yellow solid. It was made slurry with 5% methanol in diethyl ether and stirred for 15 min, filtered, and dried to give (S)-2-amino-3-(4-(tert-butoxycarbonyl)phenyl)propanoic acid as a white solid (105 g, yield 97%). Analysis condition E: Retention time=0.971 min; ESI-MS(+) m/z [M+H]⁺: 266.2.

### Step 5:

(S)-2-Amino-3-(4-(tert-butoxycarbonyl)phenyl)propanoic acid (122 g, 460 mmol) was dissolved in acetone (1000 mL) and then water (260 mL) and sodium bicarbonate (116 g, 1380 mmol) were added. It was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to get the desired product as a white solid (80 g, yield 35%). ¹H NMR (400 MHz, DMSO-d6) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl)methoxy)carbonyl)amino)-3-iodopropanoate

### Scheme:

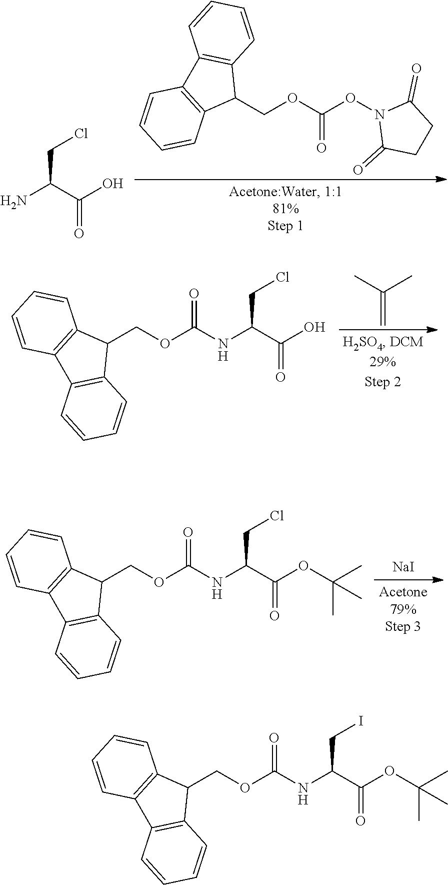

### Step 1:

To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl)methoxy)carbonyl)amino)-3-chloropropanoic acid. The product (220 g) was taken to the next step as such.

### Step 2:

A solution of (R)-2-((((9H-fluoren-9-yl)methoxy)carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H2SO₄ (57.7 mL, 1082 mmol) was added and the mixture was stirred at RT overnight. To the reaction mixture was added water (500 mL). The layers were separated and the aqueous layer was extracted DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl)methoxy)carbonyl)amino)-3-chloropropanoate (83 g, 182 mmol, 29% yield).

### Step 3:

To a solution of (R)-tert-butyl 2-((((9H-fluoren-9-yl)methoxy)carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl)methoxy)carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl)methoxy)carbonyl)amino)-3-(2-methyl-1H-indol-3-yl)propanoic Acid

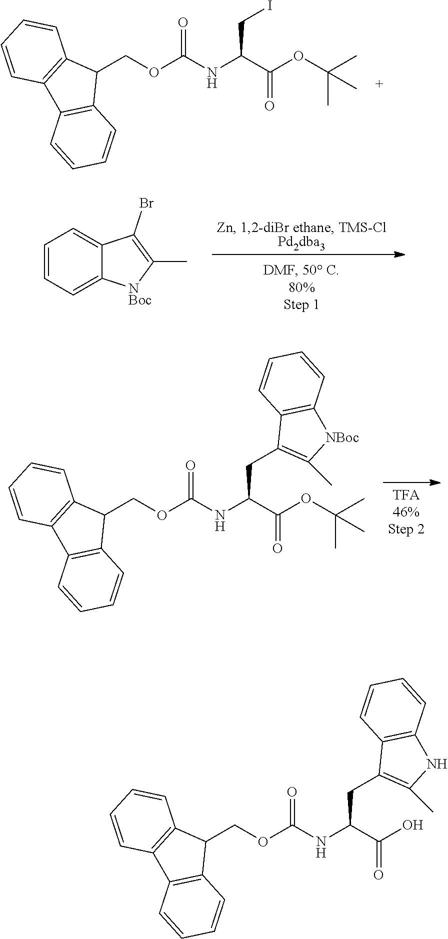 [SMILES: CC(C)(C)OC(=O)[C@H](CI)NC(=O)OCC1c2ccccc2-c2ccccc21 ; Cc1c(Br)c2ccccc2n1C(=O)OC(C)(C)C ; "Zn, 1,2-diBr ethane, TMS-ClPd2dba3" ; Cc1c(C[C@H](NC(=O)OCC2c3ccccc3-c3ccccc32)C(=O)OC(C)(C)C)c2ccccc2n1C(=O)OC(C)(C)C ; Cc1[nH]c2ccccc2c1C[C@H](NC(=O)OCC1c2ccccc2-c2ccccc21)C(=O)O]

### Step 1:

In a 100-ml three-neck, flame-dried, nitrogen-purged round-bottomed flask, zinc (2.319 g, 35.5 mmol) was added under argon atmosphere and the flask was heated to 150° C. using a hot gun and was purged with argon. To the reaction flask, DMF (50 mL) was added followed by the addition of 1,2-dibromoethane (0.017 mL, 0.20 mmol) and TMS-Cl (0.026 mL, 0.20 mmol) under argon atmosphere and then stirred for 10 min. To the reaction mixture (R)-tert-butyl 2-((((9H-fluoren-9-yl)methoxy)carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone)dipalladium(0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through diatomaceous earth (Celite®). The organic phase was washed with sat. NH₄Cl (250 mL), water (2×200 mL), and sat. NaCl (aq) (250 mL), dried over anhydrous Na₂SO₄(s), concentrated, and dried under vacuum to afford the crude compound (19 g). It was purified through ISCO flash chromatography using 330 g redisep column and the product was eluted with 7 to 9% of ethyl acetate in petroleum ether. The above reaction and purification were repeated. The pure fractions were concentrated to give tert-butyl (S)-3-(2-((((9H-fluoren-9-yl)methoxy)carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M-Boc-tBu+H]⁺: 441.2.

### Step 2:

In a 25-ml multi neck, round-bottomed flask, DCM (65 mL) was added followed by (S)-tert-butyl 3-(2-((((9H-fluoren-9-yl)methoxy)carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in cold room to give a brown colored solid (crude weight: 6.5 g). It was purified via reverse phase flash chromatography, and the pure fractions were concentrated to obtain the desired final product as an off-white powder (2.3 g, 46%). ¹H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl)methoxy)carbonyl)amino)-3-(7-methyl-1H-indol-3-yl)propanoic Acid

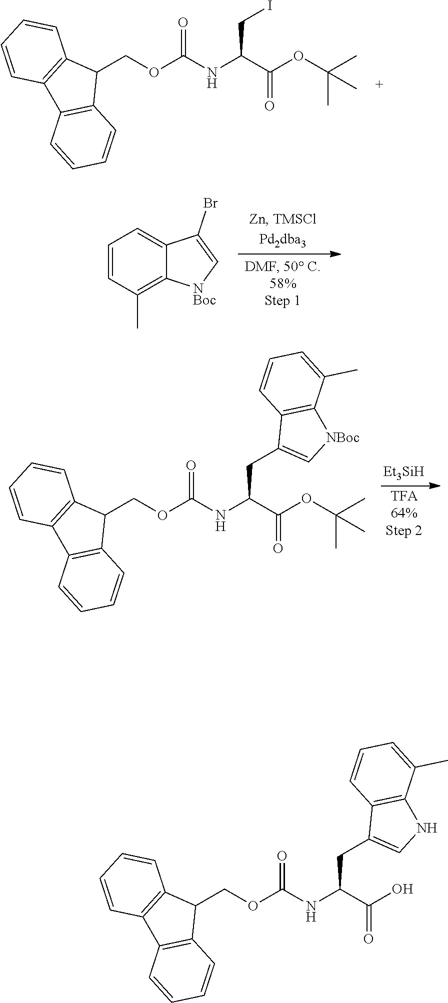

### Step 1:

In a 50-ml round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min, (R)-tert-butyl 2-((((9H-fluoren-9-yl)methoxy)carbonyl)amino)-3-iodopropanoate (2.0 g, 4.05 mmol) was added and the reaction was stirred for 30 min. In a 50-ml round-bottomed flask equipped charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂(dba)₃ (0.093 g, 0.101 mmol). After the addition the reaction mixture was heated to 50° C. overnight. Another equivalents of Sphos and Pd₂(dba)₃ was added and heating continued for another 16 h. The reaction mixture was diluted with EtOAc (100 mL) and filtered through diatomaceous earth (Celite®). The organic phase was washed with sat. aq. NH₄Cl (100 mL), water (50 mL), and sat NaCl (100 mL), dried over anhydrous Na₂SO₄(s), concentrated, and dried under vacuum. After purification by flash chromatography the desired tert-butyl (S)-3-(2-((((9H-fluoren-9-yl)methoxy)carbonyl)amino)-3-(tert-butoxy)-3-oxopropyl)-2-methyl-1H-indole-1-carboxylate was obtained in 58% yield.

### Step 2:

Final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(7-methyl-1H-indol-3-yl)propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d6) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H), 0.02-−0.06 (m, 1H).

### Preparation of (S)-2-((((9H-fluoren-9-yl)methoxy)carbonyl)amino)-3-(quinolin-6-yl)propanoic

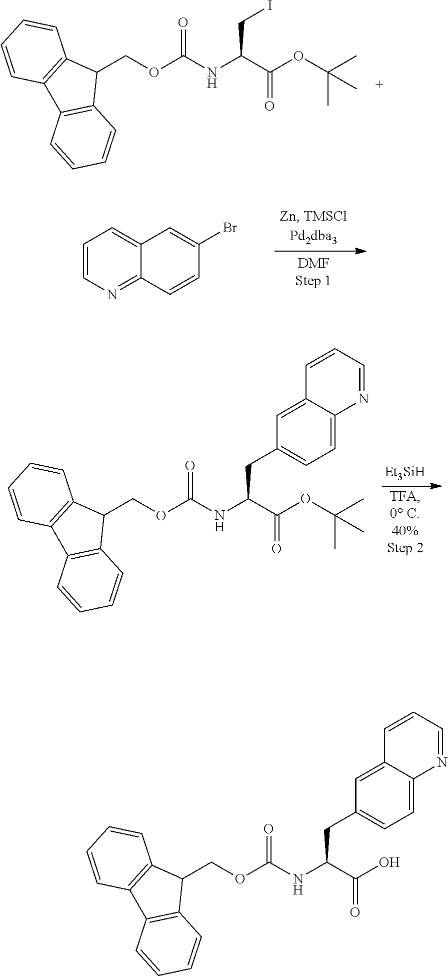

### Step 1:

In a 25-ml round bottom flask, dry zinc (2.32 g, 35.5 mmol) was charged and argon was flashed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl)methoxy)carbonyl)amino)-3-iodopropanoate (5.0 g, 10.14 mmol) was added and the reaction was stirred for 30 min.

In a 250-ml round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl)methoxy)carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at rt for 5 h and then heated to 50° C. for 16 h. It was cooled to rt and filtered through diatomaceous earth (Celite®) and rinsed with ethyl acetate. The solution was concentrated on rotovap. Purification by flash chromatography gave the desired compound as a thick brown liquid in quantitative yields. Analysis condition E: Retention time=3.47 min; ESI-MS(+) m/z [M+H]⁺: 495.2.

### Step 2:

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(quinolin-6-yl)propanoic acid as a beige solid in 40% yield after solid-liquid extraction with diethyl ether and water. ¹H NMR (300 MHz, DMSO-d6) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl)methoxy)carbonyl)amino)-3-(isoquinolin-6-yl)propanoic

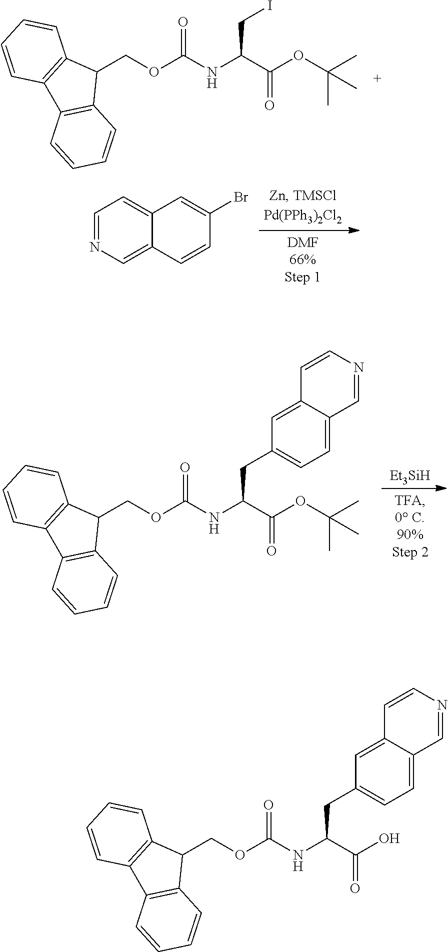

### Step 1:

In a 50-ml three neck flame-dried round bottom flask zinc (1.392 g, 21.28 mmol) was added under argon atmosphere and the flask was heated to 150° C. using a hot gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl)methoxy)carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through diatomaceous earth (Celite®) and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl (S)-2-((((9H-fluoren-9-yl)methoxy)carbonyl)amino)-3-(isoquinolin-6-yl)propanoate (2.0 g, 66%) was obtained as a yellow gum. Analysis condition B: Retention time=2.46 min; ESI-MS(+) m/z [M+H]⁺: 495.3.

### Step 2:

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(isoquinolin-6-yl)propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl)methoxy)carbonyl)amino)-3-(isoquinolin-4-yl)propanoic Acid

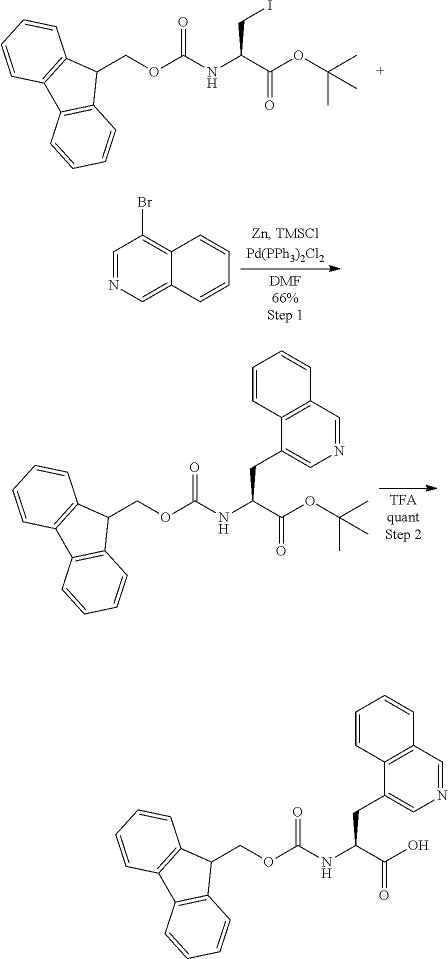 [SMILES: CC(C)(C)OC(=O)[C@H](CI)NC(=O)OCC1c2ccccc2-c2ccccc21 ; Brc1cncc2ccccc12 ; CC(C)(C)OC(=O)[C@H](Cc1cncc2ccccc12)NC(=O)OCC1c2ccccc2-c2ccccc21 ; O=C(N[C@@H](Cc1cncc2ccccc12)C(=O)O)OCC1c2ccccc2-c2ccccc21]

### Step 1:

To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was for 10 min. (R)-tert-butyl 2-((((9H-fluoren-9-yl)methoxy)carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone)dipalladium(0) (0.23 g, 0.25 mmol) and 4-bromoisoquinoline (2.11 g, 10.14 mmol) were added sequentially and the reaction was heated to 50° C. for 16 h. The reaction mixture was cooled to rt and treated with saturated ammonium chloride solution (200 mL). The crude was diluted with the ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl (S)-2-((((9H-fluoren-9-yl)methoxy)carbonyl)amino)-3-(isoquinolin-4-yl)propanoate (2.5 g, 50%). Analysis condition E: Retention time=3.44 min; ESI-MS(+) m/z [M+H]⁺: 495.2.

### Step 2:

The final product was obtained following the same procedure of (S)-2-((((9H-fluoren-9-yl)methoxy)carbonyl)amino)-3-(2-methyl-1H-indol-3-yl)propanoic acid. TFA hydrolysis afforded the desired (S)-2-((((9H-fluoren-9-yl)methoxy)carbonyl)amino)-3-(isoquinolin-4-yl)propanoic acid as an off white solid in quantitative yield after purification diethyl ether trituration. ¹H NMR (400 MHz, DMSO-d6) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl)methoxy)carbonyl)amino)-3-(4-(tert-butoxy)-3,5-difluorophenyl)propanoic Acid

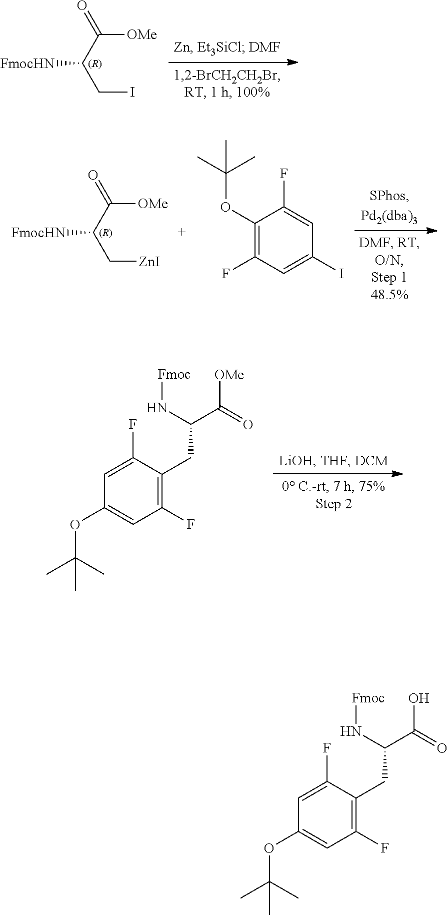

### Step 1:

The compound was prepared following the same procedure of tert-butyl (S)-2-((((9H-fluoren-9-yl)methoxy)carbonyl)amino)-3-(isoquinolin-4-yl)propanoate. First Negishi coupling with methyl (R)-2-((((9H-fluoren-9-yl)methoxy)carbonyl)amino)-3-iodopropanoate at 50° C. afforded the desired methyl (S)-2-((((9H-fluoren-9-yl)methoxy)carbonyl)amino)-3-(4-(tert-butoxy)-2,6-difluorophenyl)propanoate (5.5 g, 48.5% yield) after purification by flash chromatography.

Analysis condition E: Retention time=3.99 min; ESI-MS(+) m/z [M+NH₄]⁺: 527.2.

### Step 2:

In a multi-neck round bottom flask methyl (S)-2-((((9H-fluoren-9-yl)methoxy)carbonyl)amino)-3-(4-(tert-butoxy)-3,5-difluorophenyl)propanoate (11 g, 21.59 mmol) was added followed by the addition of tetrahydrofuran (132 mL) under nitrogen atmosphere at RT. The reaction mixture was cooled to 0° C. and LiOH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g redisep column, the product was eluted with 20% of ethyl acetate in petroleum ether. The reactions were concentrated to give (S)-2-((((9H-fluoren-9-yl)methoxy)carbonyl)amino)-3-(4-(tert-butoxy)-3,5-difluorophenyl)propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl3, 400 MHz) d 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of (S)-2-((((9H-fluoren-9-yl)methoxy)carbonyl)amino)-3-(isoquinolin-8-yl)propanoic Acid

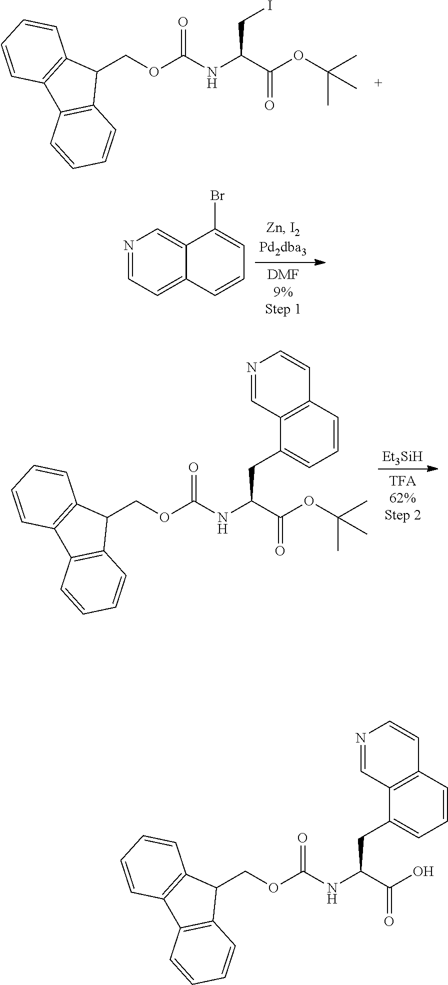

### Step 1:

Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl)methoxy)carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50 C for 4 h. under a positive pressure of nitrogen. The reaction mixture was cooled to rt, diluted with EtOAc (200 mL) and passed through diatomaceous earth (Celite®). The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford (S)-tert-butyl 2-((((9H-fluoren-9-yl)methoxy)carbonyl)amino)-3-(isoquinolin-8-yl)propanoate (380 mg, 0.768 mmol, 19.21% yield). Analysis condition G: Retention time=2.59 min; ESI-MS(+) m/z [M+H]⁺: 495.3.

### Step 2:

(S)-tert-Butyl 2-((((9H-fluoren-9-yl)methoxy)carbonyl)amino)-3-(isoquinolin-8-yl)propanoate (380 mg, 0.768 mmol) was placed in 50-ml round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl)methoxy)carbonyl)amino)-3-(isoquinolin-8-yl)propanoic acid (320 mg, 0.712 mmol, 93% yield) as an off white solid. ¹H-NMR: (400 MHz, DMSO-d6) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]⁺: 439.2 with 97.5% purity.

### Preparation of (S)-2-((((9H-fluoren-9-yl)methoxy)carbonyl)amino)-3-(7-fluoro-1H-indol-3-yl)propanoic Acid

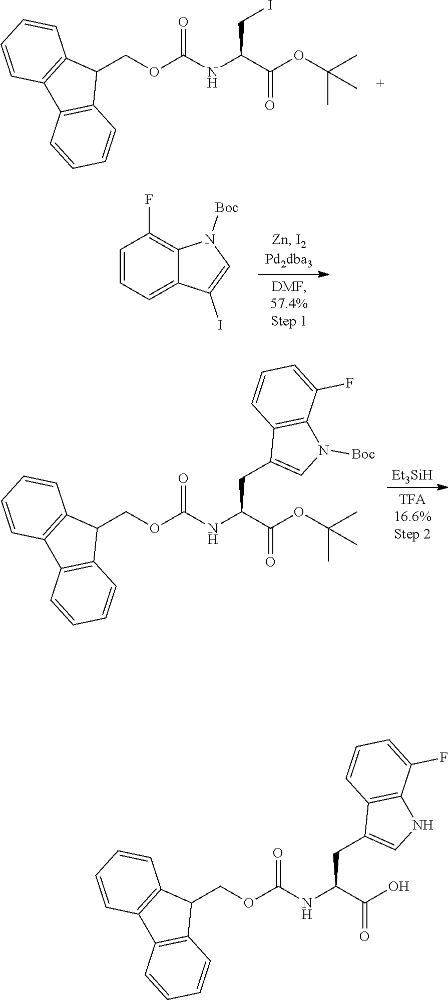

### Step 1:

Synthesis of tert-butyl 6-fluoro-3-iodo-1H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68 (s 1H), 7.30-7.34 (m, 1H), 7.03-7.08 (m, 1H), 1.66 (s, 9H)

### Step 2:

Compound was prepared following the same procedure of (S)-tert-butyl 2-((((9H-fluoren-9-yl)methoxy)carbonyl)amino)-3-(isoquinolin-8-yl)propanoate. First Negishi coupling at 50° C. afforded the desired tert-butyl (S)-3-(2-((((9H-fluoren-9-yl)methoxy)carbonyl)amino)-3-(tert-butoxy)-3-oxopropyl)-7-fluoro-1H-indole-1-carboxylate (690 mg, 1.149 mmol, 57.4% yield) after purification by flash chromatography.

Analysis condition H: Retention time=3.885 min; ESI-MS(+) m/z [M-Boc-tBu+H]⁺: 445.2

### Step 3

Final product was obtained following the same procedure of (S)-2-((((9H-fluoren-9-yl)methoxy)carbonyl)amino)-3-(isoquinolin-8-yl)propanoic acid. TFA hydrolysis afforded the desired (S)-2-((((9H-fluoren-9-yl)methoxy)carbonyl)amino)-3-(7-fluoro-1H-indol-3-yl)propanoic acid as an off white powder (96 mg, 0.191 mmol, 16.63% yield) after purification by reverse phase prep HPLC (Column: 80 g size, Silisep C18, 19×150 mm, 5 μm, Mobile phases: A=10 mM ammonium acetate in water, B=MeoH. 15 mL/min flow Gradient: 0-20 min, 5-30% B, 20-55 min, 30-80% B, 55-60 min, 80-100% B, held at 100% B for 5 min. Compound was eluted at 75% B) followed by lyophilization.

Analysis condition F: Retention time=1.367 min; ESI-MS(+) m/z [M+H]⁺: 445.3. ¹H-NMR (400 MHz, DMSO-d6) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3 S)-2-((((9H-fluoren-9-yl)methoxy)carbonyl)amino)-3-(1-(tert-butoxycarbonyl)-1H-indol-3-yl)butanoic Acid

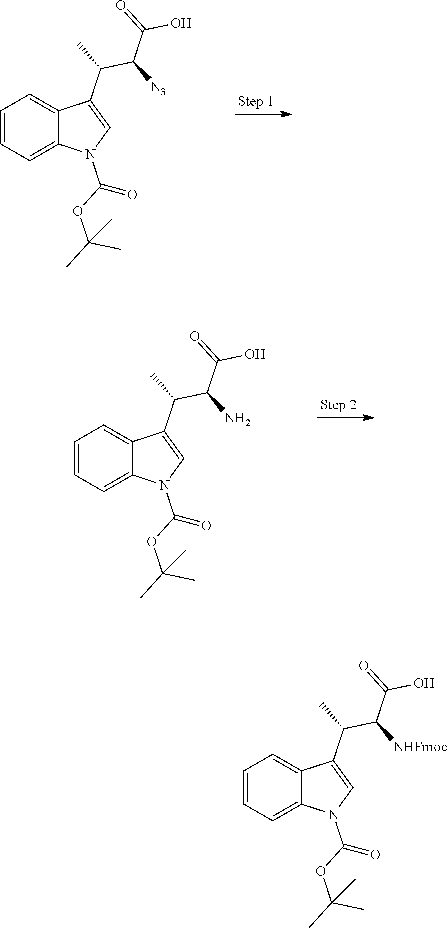

Compound (2S,3S)-2-azido-3-(1-(tert-butoxycarbonyl)-1H-indol-3-yl)butanoic acid was prepared following the procedure reported in Tetrahedron Letters 2001, 42, 4601-4603. The azide reduction step used different conditions as detailed below.

### Step 1:

To a solution of (2S,3S)-2-azido-3-(1-(tert-butoxycarbonyl)-1H-indol-3-yl)butanoic acid (1000 mg, 2.90 mmol) in THF (58 mL) was added platinum(IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through diatomaceous earth (Celite®). The solvent was removed under vacuum and the crude residue was redissolved in EtOH. This solution was filtered through diatomaceous earth (Celite®) to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

### Step 2:

To a solution of (2S,3S)-2-amino-3-(1-(tert-butoxycarbonyl)-1H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et3N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d6) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H). Analysis condition B: Retention time=1.27 min; ESI-MS(+) m/z [M+H]⁺: not observed.

### Preparation of (S)-2-((((9H-fluoren-9-yl)methoxy)carbonyl)amino)-3-(6-(o-tolyl)pyridin-3-yl)propanoic Acid

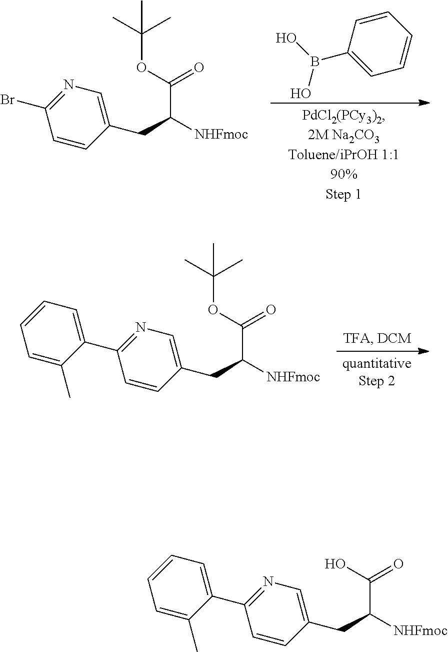

### Step 1:

To a stirred solution of tert-butyl (S)-2-((((9H-fluoren-9-yl)methoxy)carbonyl)amino)-3-(6-bromopyridin-3-yl)propanoate (1750 mg, 3.35 mmol) in toluene/iPrOH (1:1, v:v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine)palladium(II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc:DCM (1:9) as eluant lead to tert-butyl (S)-2-((((9H-fluoren-9-yl)methoxy)carbonyl)amino)-3-(6-(o-tolyl)pyridin-3-yl)propanoate (1.81 g, 3.39 mmol, 90%) as a colorless oil.

### Step 2:

(S)-2-((((9H-Fluoren-9-yl)methoxy)carbonyl)amino)-3-(6-(o-tolyl)pyridin-3-yl)propanoate (1750 mg, 3.19 mmol) was dissolved in trifluoroacetic acid (5.00 mL) and the reaction was allowed to stir at room temperature for two hours. The reaction was brought to dryness on rotovap and the crude product was dissolved in diethyl ether and 1M HCl in diethyl ether. The mixture was sonicated for 2 hours to give a white solid. The product was isolated by filtration and washed with water to give (S)-2-((((9H-fluoren-9-yl)methoxy)carbonyl)amino)-3-(6-(o-tolyl)pyridin-3-yl)propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl)methoxy)carbonyl)amino)-3-(4′-acetamido-[1,1′-biphenyl]-4-yl)propanoic Acid

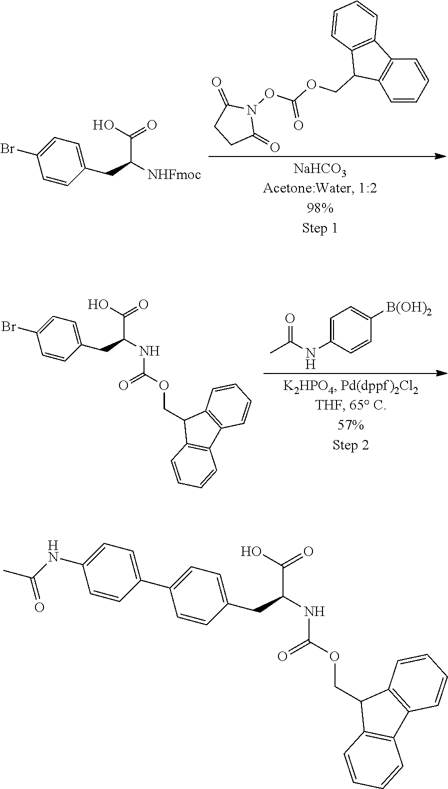

### Step 1:

A 5.0-1 multi-neck round-bottomed flask was charged with (S)-2-amino-3-(4-bromophenyl)propanoic acid (150.0 g, 615 mmol), Fmoc-OSu (207 g, 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

### Step 2:

To a stirred solution of (S)-2-((((9H-fluoren-9-yl)methoxy)carbonyl)amino)-3-(4-bromophenyl)propanoic acid (1.0 g, 2.144 mmol) and (4-acetamidophenyl)boronic acid (0.576 g, 3.22 mmol) with THF (50 mL) in 150-ml pressure tube, Argon was purged for 5 min. Potassium phosphate, tribasic (1.366 g, 6.43 mmol) was then added and the purging was continued for another 5 min. 1,1′-bis(di-tert-butylphosphino)ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to get the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d6) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

### Synthesis of Aryl/Heteroaryl Substituted Phenylalanines

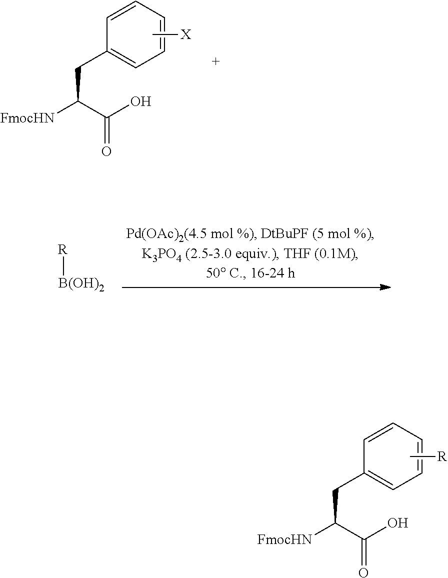

### General Procedures for Suzuki-Miyaura Coupling (SMC) Reactions in Scheme 1.

To a N₂-flushed 20-mL scintillation vial equipped with a magnetic stir bar was added Fmoc-halo-Phe-OH (0.5 mmol), boronic acid (1.5-2.5 equiv.), and anhydrous THF (6 mL). The suspension was degassed by bubbling N₂ into the vial for several minutes. Palladium(II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl)propanoic Acid

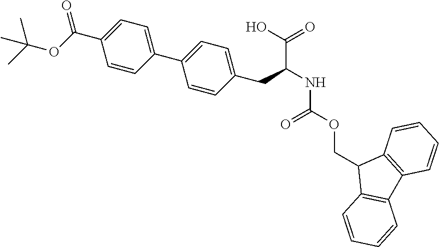 [SMILES: CC(C)(C)OC(=O)c1ccc(-c2ccc(C[C@H](NC(=O)OCC3c4ccccc4-c4ccccc43)C(=O)O)cc2)cc1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl)methoxy)carbonyl)amino)-3-(3′-(tert-butoxycarbonyl)-[1,1′-biphenyl]-4-yl)propanoic Acid

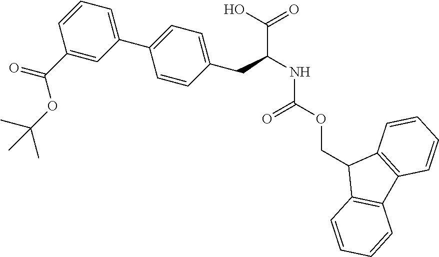 [SMILES: CC(C)(C)OC(=O)c1cccc(-c2ccc(C[C@H](NC(=O)OCC3c4ccccc4-c4ccccc43)C(=O)O)cc2)c1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 85% (240 mg); off-white solids. ¹H NMR (500 MHz, DMSO-d6) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁻ 581.26461, found at 581.26474, mass difference 0.218 ppm.

### Preparation of (S)-2-((((9H-fluoren-9-yl)methoxy)carbonyl)amino)-3-(4-boronophenyl)propanoic Acid

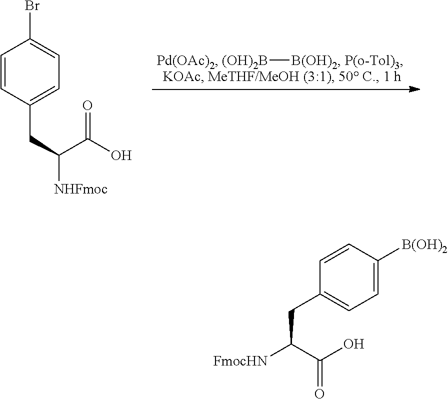

To a 75-ml pressure bottle (S)-2-((((9H-fluoren-9-yl)methoxy)carbonyl)amino)-3-(4-bromophenyl)propanoic acid (6.0 g, 12.87 mmol) and 2-methyl THF (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol) were added every in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through diatomaceous earth (Celite®) and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through diatomaceous earth (Celite®), concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl)methoxy)carbonyl)amino)-3-(4-boronophenyl)propanoic acid (4.0 g, 9.28 mmol, 72.1% yield) as a white solid. LCMS: 432.1 (M+H), tr=0.82 min. ¹H NMR (500 MHz, DMSO-d6) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl)methoxy)carbonyl)amino)-3-(4′-fluoro-[1,1′-biphenyl]-4-yl)propanoic Acid

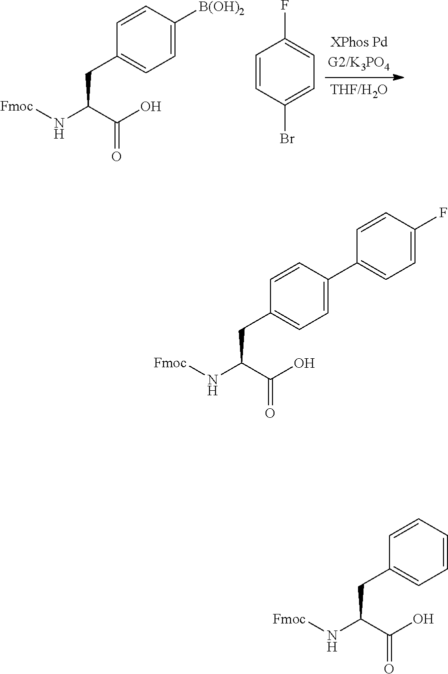

To a stirred solution of (S)-2-((((9H-fluoren-9-yl)methoxy)carbonyl)amino)-3-(4-boronophenyl)propanoic acid (217.5 mg, 0.504 mmol), 1-bromo-4-fluorobenzene (0.083 mL, 0.757 mmol) and XPhos Pd G2 (9.7 mg, 0.012 mmol) in THF (1 mL) at rt was added 0.5 M aqueous K₃PO₄ (2 mL, 1.000 mmol). N₂ was purged with vacuum three times and the mixture was stirred at 80° C. for 16 h. The mixture was cooled to rt. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash R_{f}, gradient of 0% to 20% MeOH/CH₂Cl₂ over 15 column volumes, RediSep SiO₂ 40 g). Fractions containing the desired product were collected and concentrated to give (S)-2-((((9H-fluoren-9-yl)methoxy)carbonyl)amino)-3-(4′-fluoro-[1,1′-biphenyl]-4-yl)propanoic acid (206.1 mg, 0.43 mmol, 85% yield) as a cream solid: HPLC:RT=1.04 min (Waters Acquity UPLC BEH C18 1.7 um 2.1×50 mm, CH₃CN/H₂O/0.05% TFA, 1 min. gradient, wavelength=254 nm); MS (ES): m/z=482 [M+H]⁺. ¹H NMR (499 MHz, DMSO-d6) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl)methoxy)carbonyl)amino)-3-(3′,5′-difluoro-[1,1′-biphenyl]-4-yl)propanoic Acid

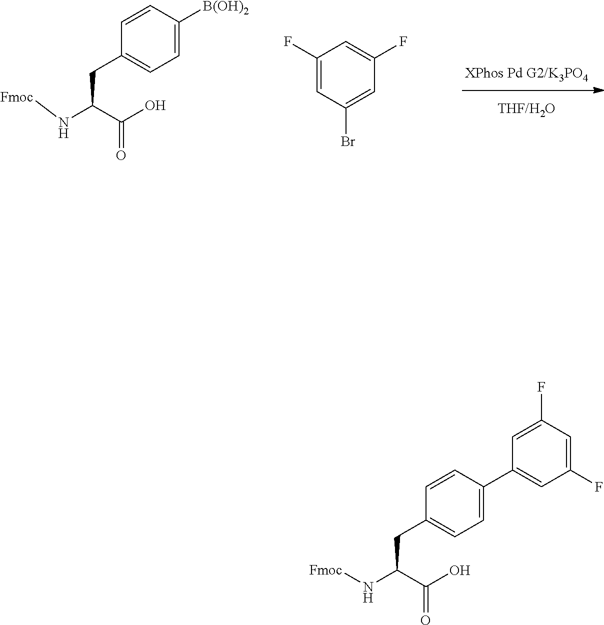

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,5′-difluoro-[1,1′-biphenyl]-4-yl)propanoic acid (197.1 mg, 0.40 mmol, 78% yield) as a colorless solid after purification by flash chromatography. HPLC:RT=1.06 min (Waters Acquity UPLC BEH C18 1.7 um 2.1×50 mm, CH₃CN/H₂O/0.05% TFA, 1 min. gradient, wavelength=254 nm); MS (ES): m/z=500 [M+H]⁺. ¹H NMR (499 MHz, DMSO-d6) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl)methoxy)carbonyl)amino)-3-(3′,4′,5′-trifluoro-[1,1′-biphenyl]-4-yl)propanoic Acid

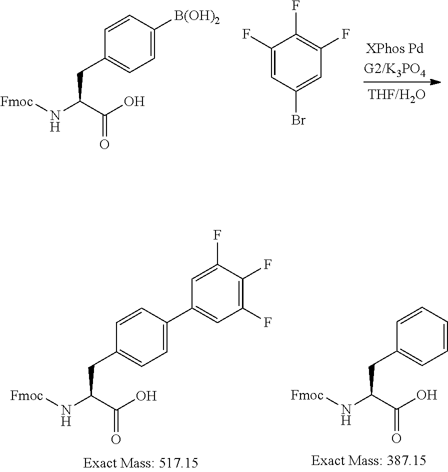

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,4′,5′-trifluoro-[1,1′-biphenyl]-4-yl)propanoic acid (218.5 mg, 0.422 mmol, 84% yield) as a colourless solid after purification by flash chromatography. HPLC:RT=1.466 min (Shimadzu UPLC with Waters Acquity BEH C18 1.7 um 2.1×50 mm column, CH₃CN/H₂O/0.1% TFA, 3 min. gradient, wavelength=254 nm); MS (ES): m/z=556. ¹H NMR (499 MHz, DMSO-d6) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

Scheme. General Procedure for Photoredox Reaction.

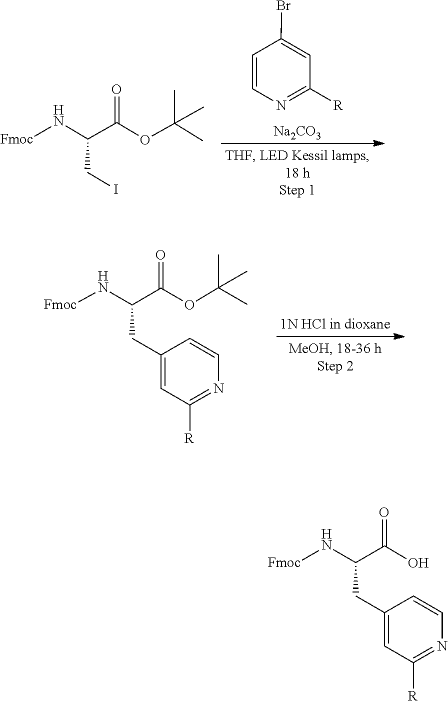

Ir[dF(CF₃)ppy₂]₂(dtbbpy)PF₆ (0.018 g, 0.016 mmol, 1 mol %), tert-butyl (R)-2-((((9H-fluoren-9-yl)methoxy)carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane)silane (0.278 g, 1.596 mmol, 1.00 equiv) were charged into an oven-dried 40-mLlpressure-relief screw cap vial. The vial was capped, purged with nitrogen, diluted with THF (45.0 mL), and then sonicated. In a separate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complex solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on rotovap. The crude product was dissolved in a minimum amount of methanol and dry loaded on silica gel column for purification.

### Preparation of (2S)-2-({[(9H-fluoren-9-yl)methoxy]carbonyl}amino)-3-(2-methoxypyridin-4-yl)propanoic Acid

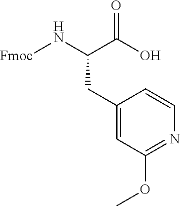

The mixture was rotovaped onto silica gel, purified by isco using 10% to 80% EtOAc/Hexanes. The fractions were pooled and concentrated to obtain the desired product as a clear oil (237 mg, 100%)

Analysis conditions D: Retention time 1.74 min; ES+ 475.1.

### Preparation of ((S)-2-((((9H-fluoren-9-yl)methoxy)carbonyl)amino)-3-(4-(trifluoromethoxy)phenyl)propanoic Acid

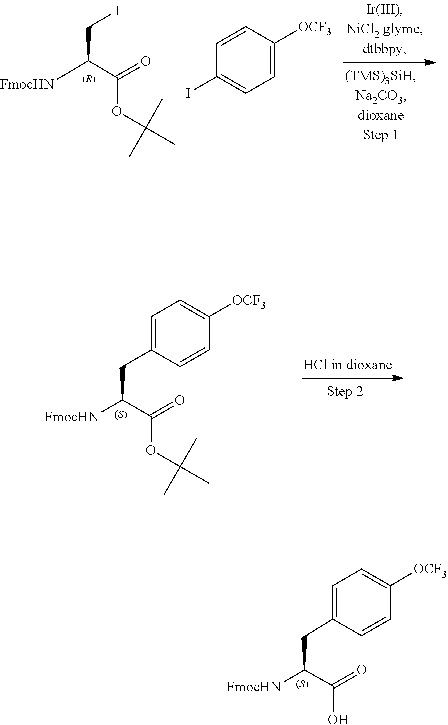

### Step 1:

In 4 separate 40-ml vials was placed Ir(dF(CF₃)ppy)₂(dtbbpy)PF₆ (5.6 mg, 4.99 μmol) and Na₂CO₃ (249 mg, 2.35 mmol) in dioxane (18 mL), and was fitted with a teflon screw cap and a stir bar. To the mixture was added 1-iodo-4-(trifluoromethoxy)benzene (0.16 mL, 1.02 mmol) stirred briefly, then tris(trimethylsilyl)silane (0.23 mL, 0.75 mmol) was added via syringe, and the suspension was degassed (cap on) with nitrogen for 5 min. To a separate 40-mL vial was added nickel(II) chloride ethylene glycol dimethyl ether complex (22 mg, 0.10 mmol) and 4,4′-di-tert-butyl-2,2′-bipyridine (33 mg, 0.12 mmol)ioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl)methoxy)carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, stirred for 40 h. The reactions were removed from the illumination/reactor. The blackish reaction mixtures of each vial were poured into a 500-ml erlenmeyer flask into which was added EtOAc (200 mL). The mixture was filtered through diatomaceous earth (Celite®), washed with EtOAc, and concentrated. The residue was purified by flash chromatography (Teledyne ISCO CombiFlash Rf, gradient of 0% using solvent A/B=CH₂Cl₂/EtOAcover 10 column volumes, RediSep SiO₂ 80 g loaded as DCM solution). The fractions containing the desired product were collected and concentrated to obtained the product tert-butyl (S)-2-((((9H-fluoren-9-yl)methoxy)carbonyl)amino)-3-(4-(trifluoromethoxy)phenyl)propanoate (865.2 mg, 1.64 mmol, 82% yield, only about 73% HPLC purity as a colourless oil and was used as was in the deprotection step: HPLC:RT=1.62 min (Waters Acquity UPLC BEH C18 1.7 um 2.1×50 mm, CH₃CN/H₂O/0.05%/TFA, 1 min. gradient, wavelength=254 nm); MS (ES): m/z=550 [M+23]+

### Step 2:

To a stirred solution of tert-butyl (S)-2-((((9H-fluoren-9-yl)methoxy)carbonyl)amino)-3-(4-(trifluoromethoxy)phenyl)propanoate (865.2 mg, 1.64 mmol) in dichloromethane (8.2 mL) at rt was added HCl (4M in dioxane, 8.20 mL, 32.8 mmol). The mixture was stirred at rt for 18 h. The mixture was concentrated in vacuo then dried under vacuum. The residue was dissolved in DMF (4 mL), purified on ISCO ACCQ Prep over 2 injections. The fractions containing the desire product were combined and partially concentrated on rotovap, then blown air over mixture over weekend. The residue was dissolved in CH₃CN, diluted with water, frozen, and lyophilized. To obtained the product (S)-2-((((9H-fluoren-9-yl)methoxy)carbonyl)amino)-3-(4-(trifluoromethoxy)phenyl)propanoic acid (344.1 mg, 0.73 mmol, 44.5% yield) as a colorless solid. HPLC:RT=1.38 min (Waters Acquity UPLC BEH C18 1.7 um 2.1×50 mm, CH₃CN/H₂O/0.05% TFA, 1.5 min. gradient, wavelength=254 nm); MS (ES): m/z=472 [M+1]+¹H NMR (499 MHz, DMSO-d6) ppm δ 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of (S)-2-((((9H-fluoren-9-yl)methoxy)carbonyl)amino)-3-(2,5-dimethylphenyl)propanoic Acid

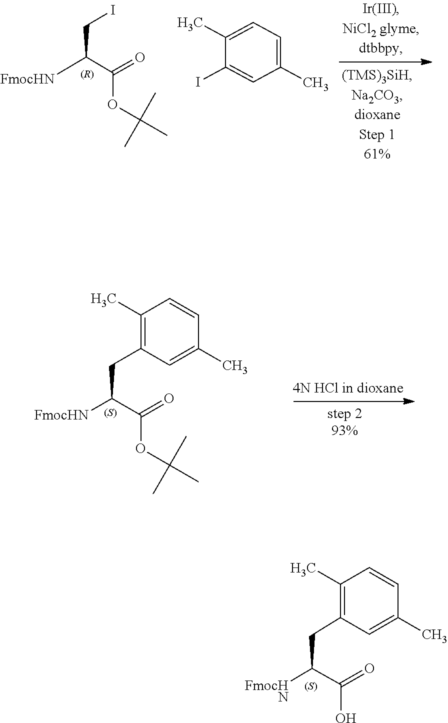

### Step 1:

Compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,5-dimethylphenyl)propanoate (140.5 mg, 0.298 mmol, 61.1% yield) after purification by flash chromatography. HPLC:RT=1.21 min (Waters Acquity UPLC BEH C18 1.7 um 2.1×50 mm, CH₃CN/H₂O/0.05% TFA, 1 min. gradient, wavelength=254 nm); Analysis condition F: Retention time=1.21 min; ESI-MS(+) m/z [M-tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H)

### Step 2:

Final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,5-dimethylphenyl)propanoic acid (115.2 mg, 0.277 mmol, 93% yield) as a cream solid after purification by reverse phase flash chromatography. HPLC:RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H)

### Preparation of (S)-2-((((9H-fluoren-9-yl)methoxy)carbonyl)amino)-3-(4-fluoro-3-methylphenyl)propanoic Acid

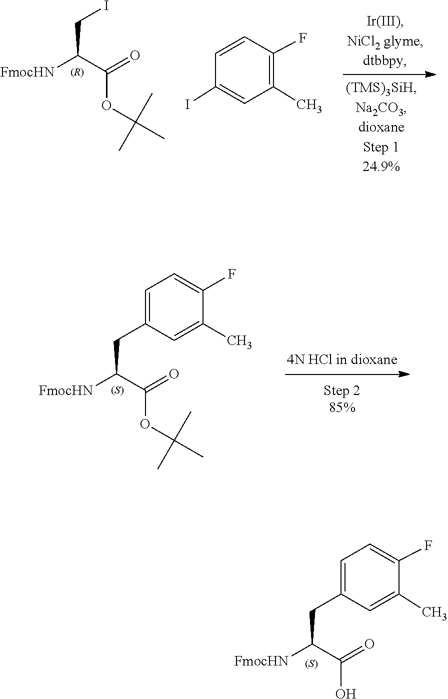

### Step 1:

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(4-fluoro-3-(trifluoromethyl)phenyl)propanoate (66.3 mg, 0.13 mmol, 24.9% yield) as a colourless solid after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=474 [M-tBu]+. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H)

### Step 2:

Final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(4-fluoro-3-methylphenyl)propanoic acid (58.3 mg, 0.139 mmol, 85% yield) as a cream solid after purification by reverse phase flash chromatography. HPLC:RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+H]⁺. ¹H NMR (499 MHz, DMSO-d6) δ 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl)methoxy)carbonyl)amino)-3-(2,4-difluoro-5-methoxyphenyl)propanoic Acid

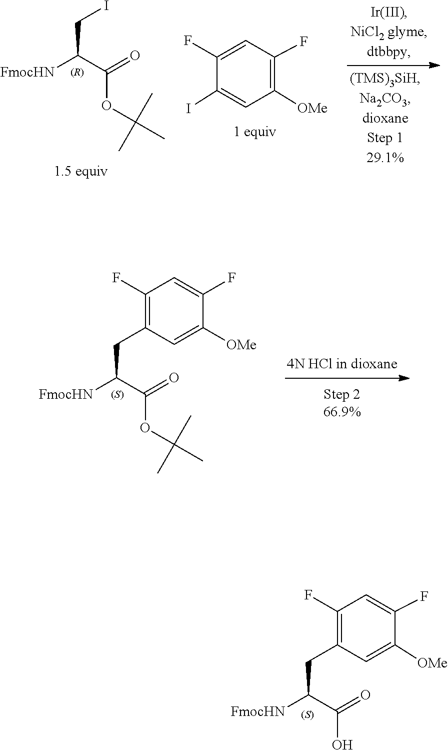

### Step 1:

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,4-difluoro-5-methoxyphenyl)propanoate (77.1 mg, 0.151 mmol, 29.1% yield as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=454 [M−t−Bu]+. ¹H NMR (499 MHz, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H)

### Step 2:

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,4-difluoro-5-methoxyphenyl)propanoic acid (45.9 mg, 0.101 mmol, 66.9% yield) as a cream solid after purification by reverse phase flash chromatography. HPLC:RT=0.99 min (Waters Acquity UPLC BEH C18 1.7 um 2.1×50 mm, CH₃CN/H₂O/0.05% TFA, 1 min. gradient, wavelength=254 nm); MS (ES): m/z=454 [M+1]+. ¹H NMR (499 MHz, DMSO-d6) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H).

### Preparation of (S)-2-((((9H-fluoren-9-yl)methoxy)carbonyl)amino)-3-(2,3-dimethylphenyl)propanoic Acid

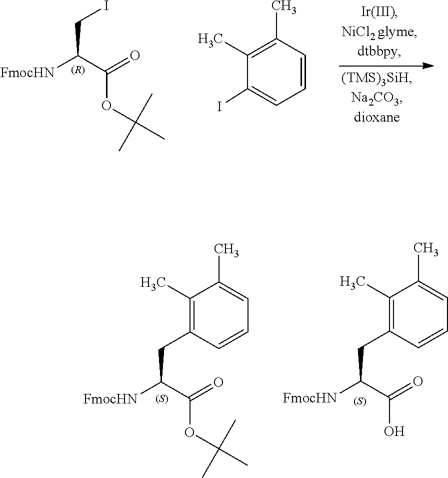

### Step 1:

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,3-dimethylphenyl)propanoate (107.5 mg, 0.228 mmol, 55.5% yield) as a tan viscous oil after purification by flash chromatography. HPLC:RT=1.21 min (Waters Acquity UPLC BEH C18 1.7 um 2.1×50 mm, CH₃CN/H₂O/0.05% TFA, 1 min. gradient, wavelength=254 nm); MS (ES): m/z=416 [M−t−Bu]+. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

### Step 2:

The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,3-dimethylphenyl)propanoic acid (72.9 mg, 0.175 mmol, 77% yield) as a cream solid after purification by reverse phase flash chromatography. HPLC:RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]⁺. ¹H NMR (499 MHz, DMSO-d6) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of (S)-2-((((9H-fluoren-9-yl)methoxy)carbonyl)amino)-3-(2-fluoro-3-methylphenyl)propanoic Acid

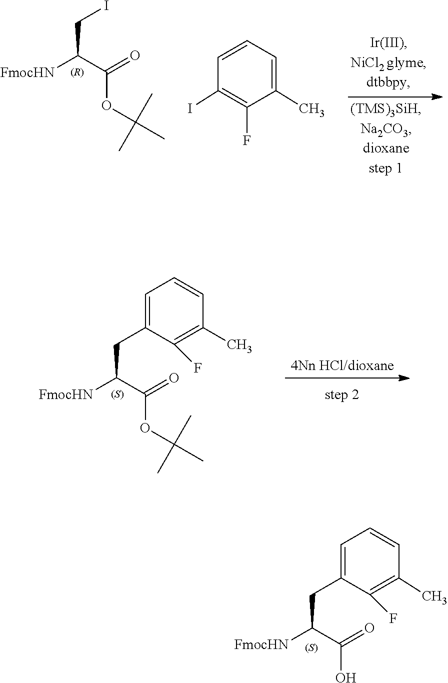

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-3-methylphenyl)propanoate (136.9 mg, LCMS showed 77% product and 23% impurity) as a viscous oil after purification by flash chromatography. Used as is, purify at after tBu hydrolysis.

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-3-methylphenyl)propanoic acid (79.7 mg, 0.190 mmol, 66.0% yield) as a cream solid after purification by reverse phase flash chromatography. HPLC:RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]. ¹H NMR (499 MHz, DMSO-d6) δ 12.79 (br s, 1H), 7.89 (d, J=7.7 Hz, 2H), 7.78 (d, J=8.6 Hz, 1H), 7.65 (dd, J=11.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). ¹H NMR and LCMS showed a 14% impurity.

### Preparation of ((S)-2-((((9H-fluoren-9-yl)methoxy)carbonyl)amino)-3-(2-fluoro-5-methylphenyl)propanoic Acid

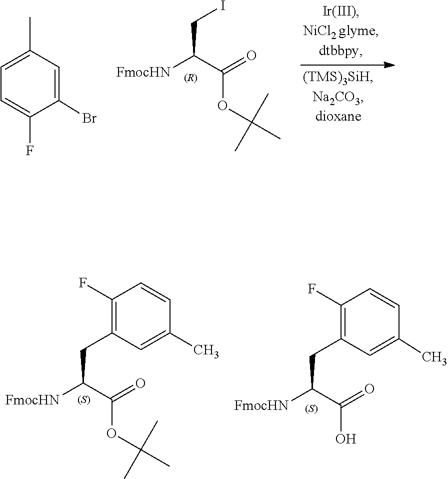

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-5-methylphenyl)propanoate (148.1 mg, 0.311 mmol, 65.4% yield) as a colourless gum after purification by flash chromatography. HPLC:RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=420 [M−t−Bu]+. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H)

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methylphenyl)propanoic acid (98.1 mg, 0.23 mmol, 75% yield) as a colourless solid after purification by reverse phase flash chromatography. HPLC:RT=1.01 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]. ¹H NMR (499 MHz, DMSO-d6) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H)

### Preparation of (S)-2-((((9H-fluoren-9-yl)methoxy)carbonyl)amino)-3-(2-fluoro-5-methoxyphenyl)propanoic Acid

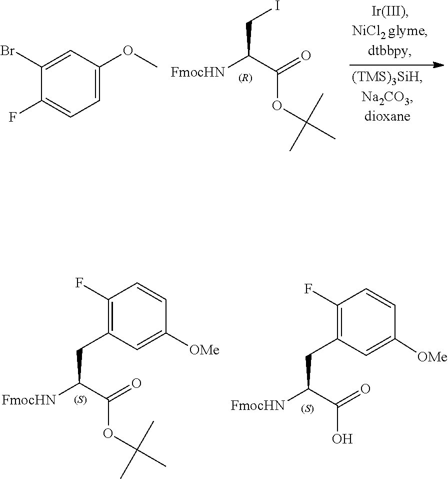

### Step 1:

The compound was prepared following the same procedure of tert-butyl (S)-2-((((9H-fluoren-9-yl)methoxy)carbonyl)amino)-3-(2-fluoro-5-methoxyphenyl)propanoate (117.7 mg, 0.24 mmol, 50.4% yield) as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=436 [M−t−Bu]+. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H)

### Step 2:

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methoxyphenyl)propanoic acid (79.5 mg, 0.183 mmol, 76% yield) as a colourless solid after purification by flash chromatography. HPLC:RT=0.98 min (Waters Acquity UPLC BEH C18 1.7 um 2.1×50 mm, CH₃CN/H₂O/0.05% TFA, 1 min. gradient, wavelength=254 nm); MS (ES): m/z=436 [M+1]+. Base peak of 214=fully deprotected amino acid fragment was also observed. ¹H NMR (499 MHz, DMSO-d6) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl)methoxy)carbonyl)amino)-3-(2-methoxy-5-methylphenyl)propanoic Acid

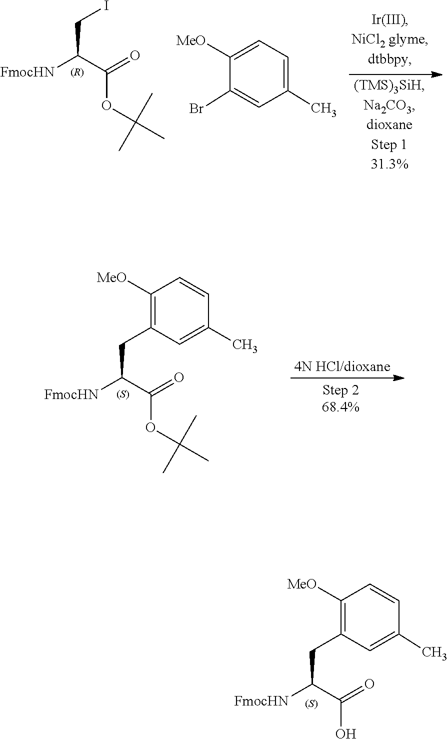

### Step 1:

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-methoxy-5-methylphenyl)propanoate (73.9 mg, 0.15 mmol, 31.3% yield) as a colourless film after purification by flash chromatography. HPLC:RT=1.20 min (Waters Acquity UPLC BEH C18 1.7 um 2.1×50 mm, CH₃CN/H₂O/0.05% TFA, 1 min. gradient, wavelength=254 nm); MS (ES): m/z=488 [M-tBu+H]⁺. ¹H NMR (499 MHz, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H)

### Step 2:

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-methoxy-5-methylphenyl)propanoic acid (44.7 mg, 0.104 mmol, 68.4% yield) as a colourless solid after purification by flash chromatography. HPLC:RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=432 [M+H]⁺. ¹H NMR (499 MHz, DMSO-d6) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J=13.5, 4.8 Hz, 1H), 2.72 (dd, J=13.4, 10.2 Hz, 1H), 2.16 (s, 3H).

### Preparation of (S)-2-((((9H-fluoren-9-yl)methoxy)carbonyl)amino)-3-hydroxy-3-methylbutanoic Acid

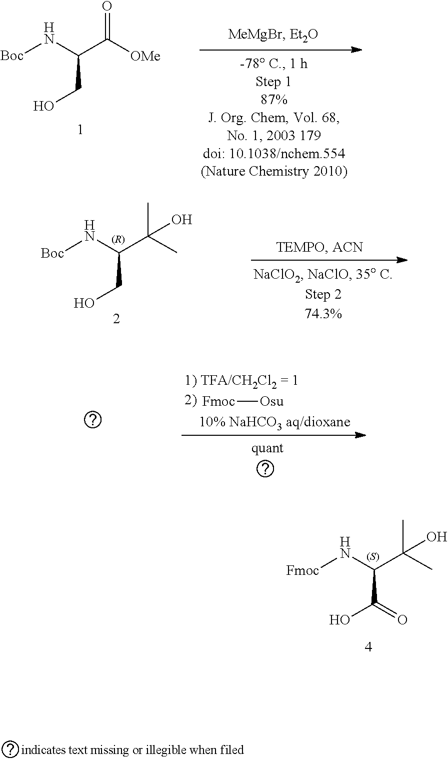

### Step 1:

To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by I2PAC. Desired fractions were eluted at 50% EtOAc:petroleum ether mixture, and were collected and concentrated at 40° C. to give tert-butyl (R)-(1,3-dihydroxy-3-methylbutan-2-yl)carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

### Step 2:

A 50-ml single neck round-bottomed flask was charged with tert-butyl (R)-(1,3-dihydroxy-3-methylbutan-2-yl)carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and the pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude (S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9 s, 3H).

### Step 3:

A 2000-mL single neck flask was charged with (S)-2-((tert-butoxycarbonyl)amino)-3-hydroxy-3-methylbutanoic acid (90 g, 386 mmol)dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude (S)-2-amino-3-hydroxy-3-methylbutanoic acid, HCl (70 g, 107% yield) as a white solid and was taken directly to the next step.

### Step 4:

To a 3000-ml multi-neck round-bottomed flask was charged (S)-2-amino-3-hydroxy-3-methylbutanoic acid, HCl (70 g, 413 mmol), dioxane (1160 mL) and water (540 mL) The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following procedure was followed: the solid was dissolved in ethyl acetate (1200 mL) and was stripped off with n-hexane (3000 mL). The slurry obtained was stirred for 10 min, filtered, dried under vacuum to give (S)-2-((((9H-fluoren-9-yl)methoxy)carbonyl)amino)-3-hydroxy-3-methylbutanoic acid (112.0 g, 74.8 yield for two steps) as a white solid.

### Preparation of (S)-2-((((9H-fluoren-9-yl)methoxy)carbonyl)amino)-3-(3,4,5-trifluorophenyl)propanoic Acid

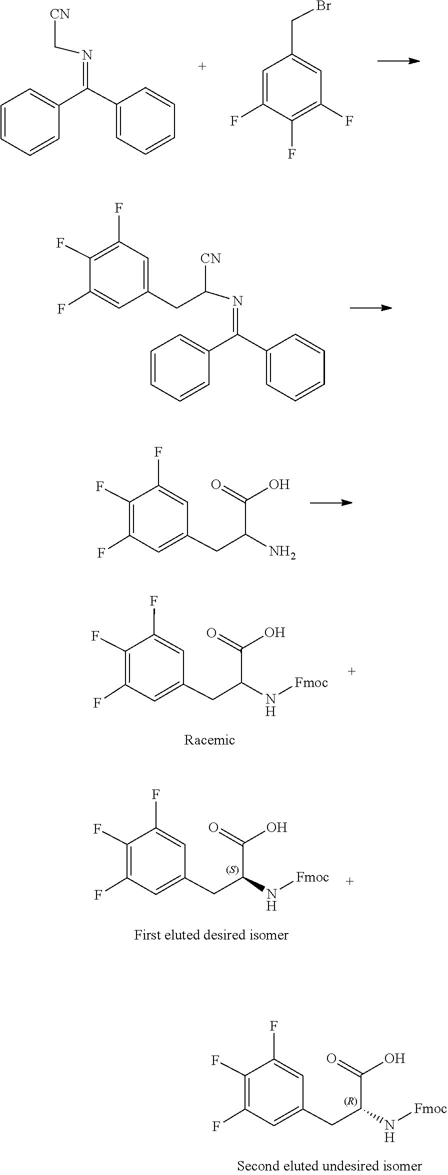

### Step 1:

To a stirred solution of 2-((diphenylmethylene)amino)acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at rt overnight. After 26 h, the reaction mixture was diluted with water (500 mL) and the DCM layer was separated. The aqueous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl)propanenitrile (140 g, 384 mmol, 85% yield) as a yellow solid. Analysis condition E: Retention time=3.78 min; ESI-MS(+) m/z [M+H]⁺: 365.2.

### Step 2:

To a stirred solution of 2-((diphenylmethylene)amino)-3-(3,4,5-trifluorophenyl)propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for next step.

### Step 3:

To the crude aqueous dioxane solution from the previous was added 10 N NaOH solution until the solution was neutral. Na₂CO₃ (438 ml, 438 mmol) was then added, followed by the addition of Fmoc-OSu (81 g, 241 mmol). The mixture was stirred at rt overnight. The aqueous solution was acidified with 1.5 N HCl till pH=2 and the solid formed was filtered, dried to afford the crude compound. It was slurried initially with 5% EtOAc/petroleum ether for 30 min and filtered. The filtered compound was further slurried with ethyl acetate for 20 min and filtered to get the crude racemic 2-((((9H-fluoren-9-yl)methoxy)carbonyl)amino)-3-(3,4,5-trifluorophenyl)propanoic acid (90 g, 204 mmol, 93% yield) as an off-white solid. This racemic compound was separated into two isomers by SFC purification to get the desired isomers. After concentration of the desired isomer, it was slurried with 5% EtOAc/petroleum ether and filtered to get (S)-2-((((9H-fluoren-9-yl)methoxy)carbonyl)amino)-3-(3,4,5-trifluorophenyl)propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ −137.56 (d, J=19.6 Hz, 2F), −166.67 (t, J=19.6 Hz, 1F). Analysis condition E: Retention time=3.15 min; ESI-MS(+) m/z [M+H]⁺: 442.2.

The other fraction was concentrated to get (R)-2-((((9H-fluoren-9-yl)methoxy)carbonyl)amino)-3-(3,4,5-trifluorophenyl)propanoic acid (40 g, 91 mmol, 41.4% yield) as an off-white solid.

### Preparation of (S)-2-((((9H-fluoren-9-yl)methoxy)carbonyl)amino)-4-(tert-butoxy)-3,3-dimethyl-4-oxobutanoic Acid

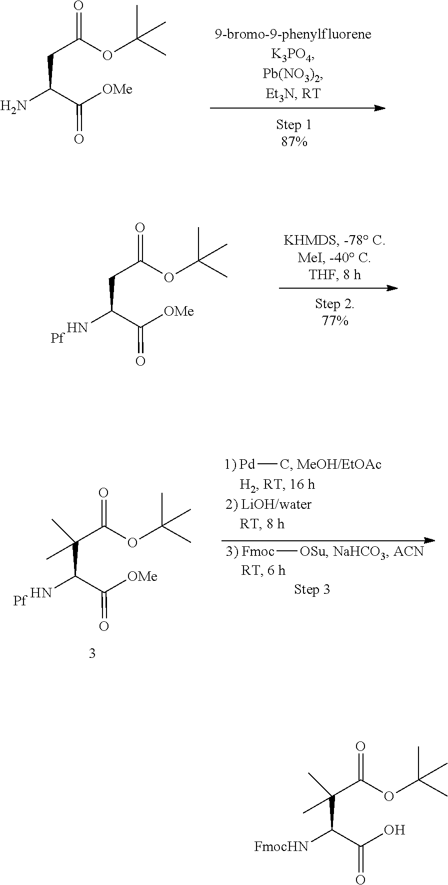

### Step 1:

To a stirred solution of 4-(tert-butyl) 1-methyl L-aspartate, HCl salt (34 g, 142 mmol) in acetonitrile (550 mL), was added lead(II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% EA in PE) and LCMS. The reaction mixture was filtered over diatomaceous earth (Celite®), washed with chloroform, and evaporated to get thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to get pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]⁺: 466.40.

### Step 2:

A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃ (200 mL), and brine. The organic layer was dried over anhydrous Na₂SO₄, evaporated in vacuo, and recrystallized from hexanes to give 1-(tert-butyl) 4-methyl (S)-2,2-dimethyl-3-((9-phenyl-9H-fluoren-9-yl)amino)succinate (18.5 g, 39.2 mmol, 77% yield) as a white solid, which was taken for next step. Analysis condition L: Retention time=2.04 min; ESI-MS(+) m/z [M+Na]⁺: 494.34.

### Step 3:

A stirred solution of 1-(tert-butyl) 4-methyl (S)-2,2-dimethyl-3-((9-phenyl-9H-fluoren-9-yl)amino)succinate (24 g, 50.9 mmol) in methanol (270 mL) and ethyl acetate (100 mL) was degassed with nitrogen. Pd—C (2.71 g, 2.54 mmol) (10% by weight) was added, and the mixture was flushed with hydrogen gas and then stirred at RT in 1-liter capacity autoclave with 50 psi overnight. The reaction mixture was filtered through diatomaceous earth (Celite®), washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

### Step 4:

To a stirred solution of 1-(tert-butyl) 4-methyl (S)-3-amino-2,2-dimethylsuccinate (11.0 g, 47.6 mmol)cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% EA in petroleum ether to get (S)-2-((((9H-fluoren-9-yl)methoxy)carbonyl)amino)-4-(tert-butoxy)-3,3-dimethyl-4-oxobutanoic acid (12.2 g, 26.9 mmol, 39.0% yield) as a white solid. ¹HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of (S)-2-((((9H-fluoren-9-yl)methoxy)carbonyl)amino)-3-(3-(tert-butoxycarbonyl)phenyl)propanoic Acid

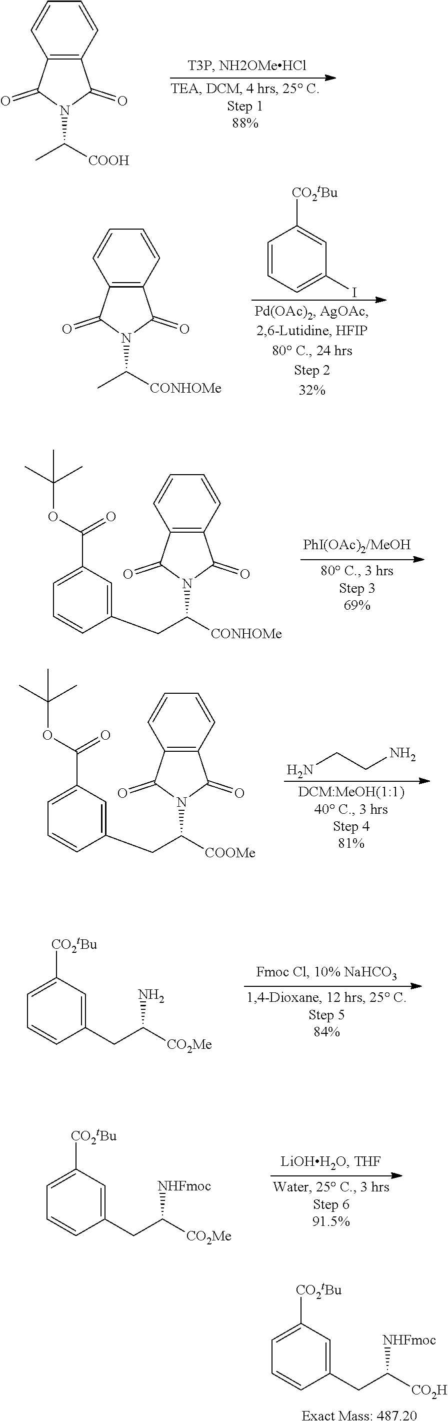

### Step 1:

To a solution of (S)-2-(1,3-dioxoisoindolin-2-yl)propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using 120 g silica column with 38 to 45% EtOAc in petroleum ether to give (S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H). (A082E-536-01)

### Step 2:

To a solution of (S)-2-(1,3-dioxoisoindolin-2-yl)-N-methoxypropanamide (20 g, 81 mmol), palladium(II) acetate (1.809 g, 8.06 mmol), silver acetate (26.9 g, 161 mmol) placed in a 1000-ml seal tube, was added tert-butyl 3-iodobenzoate (36.8 g, 121 mmol), 2,6-Lutidine (2.395 ml, 24.17 mmol), HFIP (300 ml) at 25° C. under N₂ atmosphere. The reaction was stirred for 15 min at 25° C. under N₂ and then heated Up to 80° C. for 24 h with vigorous stirring. The reaction mixture was filtered through diatomaceous earth (Celite®) and washed with DCM (200 mL). The combined organic layer was concentrated under reduced pressure. The crude product was purified via combiflash using 220 g silica column eluting with 25 to 30% EtOAc:CHCl₃ to obtain the desired product tert-butyl (S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H),

### Step 3:

To a solution of tert-butyl (S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The Reaction was concentrated under reduced pressure to get the crude product. It was purified with silica gel chromatography (100-200 mesh eluting with 20% EA: hexane) to obtain the desired compound tert-butyl (S)-3-(2-(1,3-dioxoisoindolin-2-yl)-3-methoxy-3-oxopropyl)benzoate (10 g, 24.42 mmol, 69.1% yield. ¹H NMR (CDCl₃, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H).

### Step 4:

To a solution of tert-butyl (S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% EA: hexane) to obtain the desired compound tert-butyl (S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H). (A082E-555-01)

### Step 5:

To a solution of tert-butyl (S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetyate. The organic layer was concentrated under reduced pressure to get the crude product. It was purified via silica gel chromatography (100-200 mesh eluting with 20% EA: hexane) to obtain the desired compound tert-butyl (S)-3-(2-((((9H-fluoren-9-yl)methoxy)carbonyl)amino)-3-methoxy-3-oxopropyl)benzoate (15 g, 29.9 mmol, 84% yield).

### Step 6:

To a solution of tert-butyl (S)-3-(2-((((9H-fluoren-9-yl)methoxy)carbonyl)amino)-3-methoxy-3-oxopropyl)benzoate (18.00 g, 35.9 mmol) in THF (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THE. In the basic medium the mixture was extracted with diethyl ether to remove the non polar impurities. The aqueous layer was acidified with aqueous citric acid solution and extracted with ethyl acetate. The organic layer was dried over sodium sulphate and concentrated under reduced to get the desired compound as a gummy solid which was further lyopholized to give off-white solids. (A082E-559-01&05) the desired compound Lot 1: (S)-2-((((9H-fluoren-9-yl)methoxy)carbonyl)amino)-3-(3-(tert-butoxycarbonyl)phenyl)propanoic acid (11 g, 22.56 mmol, 62.9% yield). And lot 2: (S)-2-((((9H-fluoren-9-yl)methoxy)carbonyl)amino)-3-(3-(tert-butoxycarbonyl)phenyl)propanoic acid (5 g, 10.26 mmol, 28.6% yield). 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl)methoxy)carbonyl)amino)-3-(m-tolyl)propanoic Acid

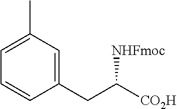

Compound was synthesized following the similar procedures of (S)-2-((((9H-fluoren-9-yl)methoxy)carbonyl)amino)-3-(3-(tert-butoxycarbonyl)phenyl)propanoic acid. Analysis condition E: Retention time=3.147 min; ESI-MS(+) m/z [M+H]⁺: 402.0. ¹H NMR (DMSO-d₆, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

### Preparation of (S)-2-((((9H-fluoren-9-yl)methoxy)carbonyl)amino)-3-(1-(tert-butoxycarbonyl)-1H-indol-4-yl)propanoic Acid

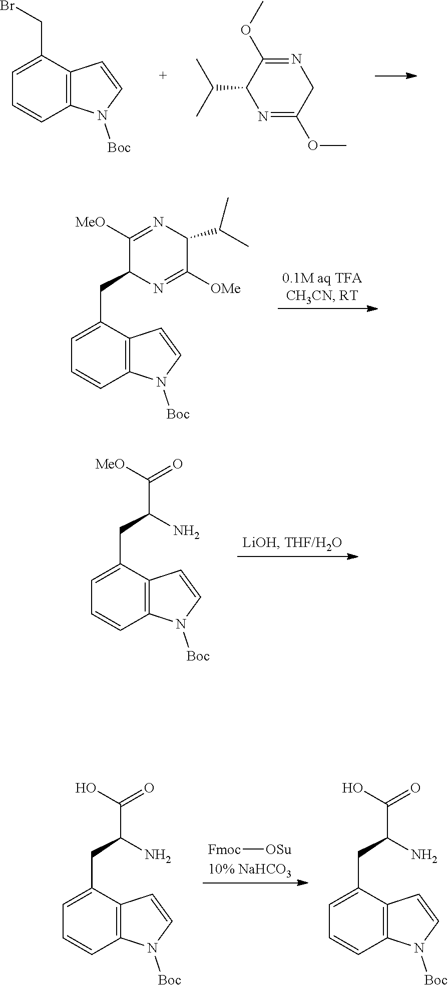

### Step 1:

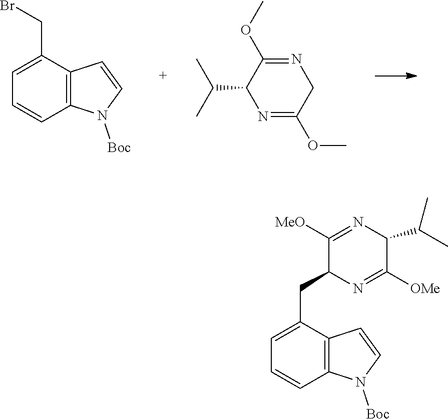 [SMILES: CC(C)(C)OC(=O)n1ccc2c(CBr)cccc21 ; COC1=N[C@H](C(C)C)C(OC)=NC1 ; COC1=N[C@H](C(C)C)C(OC)=N[C@H]1Cc1cccc2c1ccn2C(=O)OC(C)(C)C]

To a −78° C. cooled solution of (R)-2-isopropyl-3,6-dimethoxy-2,5-dihydropyrazine (29.7 g, 161 mmol) in THF (500 mL) was added n-butyllithium (77 mL, 193 mmol) slowly, and the reaction mass was stirred for 30 min. A solution of tert-butyl 4-(bromomethyl)-1H-indole-1-carboxylate (50 g, 161 mmol) in THF (500 mL) was added over a period of 10 min, and the reaction mass was stirred −78° C. for 1 h. The reaction was quenched with saturated ammonium chloride solution. The layers were separated and the aqueous layer was extracted with ethyl acetate (1000 mL) The combined organic layers were washed with brine, dried with sodium sulphate, and concentrated to get 75 g of the crude compound. The crude material of this batch was mixed with the crude compound of another patch for purification. The product fractions were concentrated to get 70 g of the required compound tert-butyl 4-(((2S,5R)-5-isopropyl-3,6-dimethoxy-2,5-dihydropyrazin-2-yl)methyl)-1H-indole-1-carboxylate as a colourless liquid.

### Step 2:

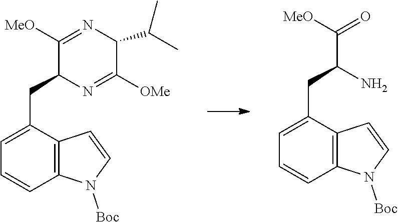 [SMILES: COC1=N[C@H](C(C)C)C(OC)=N[C@H]1Cc1cccc2c1ccn2C(=O)OC(C)(C)C ; COC(=O)[C@@H](N)Cc1cccc2c1ccn2C(=O)OC(C)(C)C]

To a 0° C. solution of tert-butyl 4-(((2S,5R)-5-isopropyl-3,6-dimethoxy-2,5-dihydropyrazin-2-yl)methyl)-1H-indole-1-carboxylate (70 g, 169 mmol) in acetonitrile (1600 mL) was added a solution of TFA (46 mL, 597 mmol) in water (500 mL), and then was stirred at room temperature for overnight. The solvent was removed and the aqueous layer was extracted with DCM (500 ml×3). The combined DCM layers were washed with brine solution, dried with sodium sulphate, and concentrated to give 54 g of tert-butyl (S)-4-(2-amino-3-methoxy-3-oxopropyl)-1H-indole-1-carboxylate (89% HPLC purity). Analysis condition E: Retention time=2.658 min; ESI-MS(+) m/z [M+H]⁺: 305.2.

### Step 3:

To a solution of tert-butyl (S)-4-(2-amino-3-methoxy-3-oxopropyl)-1H-indole-1-carboxylate (54 g, 170 mmol) in THF (1200 mL) was added a solution lithium hydroxide monohydrate (12.19 g, 509 mmol) in water (600 mL), then the reaction mass was stirred at room temperature for 30 min. THF was removed, and saturated 1N HCl solution was added to the residue to adjust pH to 5. The mixture was filtered and the solids were dried to get 45 g 73. 2% 84% of the required compound. Analysis condition E: Retention time=1.524 min; ESI-MS(+) m/z [M+H]⁺: 305.2.

### Step 4:

The mixture of (S)-2-amino-3-(1-(tert-butoxycarbonyl)-1H-indol-4-yl)propanoic acid (45 g, 129 mmol) and 10% sodium bicarbonate solution (750 mL) was stirred for 1 h, then added a solution of Fmoc-OSu (45.6 g, 135 mmol) in acetone (750 mL). The reaction was stirred at room temperature for 12 h. Acetone was removed completely. The reaction was cooled and then saturated citric acid solution was adjusted the pH to 5. The aqueous layer was extracted with ethyl acetate (500×3). The combined organic layers were washed with brine solution, dried with sodium sulphate, and concentrated to get 100 g of the crude compound. Purified the crude compound by ISCO, compound elutes with the 5% of methanol in chloroform. Concentrated the product fractions to get 40 g of the required compound. Dissolved in CH₂Cl₂ and concentrated to dryness to give (S)-2-((((9H-fluoren-9-yl)methoxy)carbonyl)amino)-3-(1-(tert-butoxycarbonyl)-1H-indol-4-yl)propanoic acid (40 g, 57.9% yield, 98% HPLC purity). Analysis condition E: Retention time=2.835 min; ESI-MS(+) m/z [M−H]⁺: 525.5. ¹H NMR (DMSO-d₆, 400 MHz), d 7.94 (d, J=8.0 Hz, 1H), 7.88 (d, J=7.6 Hz, 2H), 7.79 (d, J=7.6 Hz, 1H), 7.67 (d, J=3.6 Hz, 1H), 7.62 (d, J=7.6 Hz, 1H), 7.59 (d, J=7.6 Hz, 1H), 7.43-7.38 (m, 2H), 7.32-7.25 (m, 3H), 7.16 (d, J=7.2 Hz, 1H), 6.86 (d, J=8.0 Hz, 1H), 4.28-4.19 (m, 1H), 4.19-4.13 (m, 3H), 3.38-3.34 (m, 1H), 3.18-3.12 (m, 1H), 1.62 (s, 9H).

### Preparation ethyl (S)-5-((tert-butoxycarbonyl)amino)-2-(((S)-mesitylsulfinyl)amino)-3,3-dimethylpentanoate

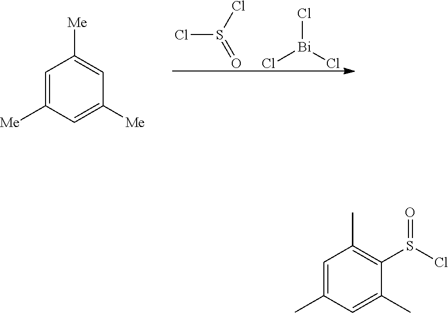

### Step 1:

The compound was synthesized using similar procedure described in reference: To a 1000-ml flask equipped with a septum inlet and magnetic stirring bar was added bismuth(III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding to an orange liquid. In order to remove the catalyst, 2000 mL of pentane were added, stirred and filtered through diatomaceous earth (Celite®), and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

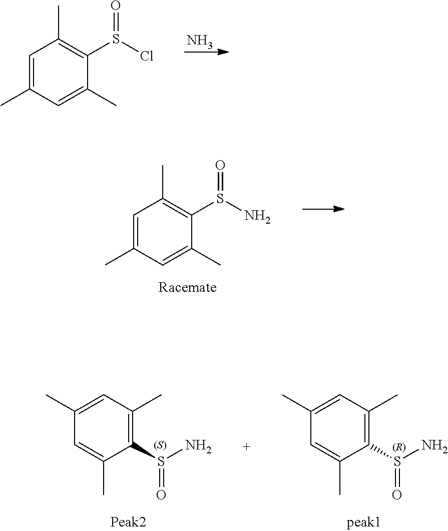

### Step 2:

The compound was synthesized using similar procedure described in reference: To a stirred solution of 2,4,6-trimethylbenzenesulfinic chloride (155 g, 765 mmol) in diethyl ether (1500 mL). After it had been cooled to −40° C. In a separate setup, (2 L multi neck RBF) taken in diethyl ether (900 mL) ammonia gas was bubbled 30 minutes at −40° C., this purged solution was added to above reaction mass at −40° C. After it had warmed to rt the reaction mixture was stirred for 2 hours and monitored by open access LCMS starting material was absent. The reaction was stirred at room temperature overnight according to given procedure. The reaction was monitored by TLC and open access LCMS (A1CE6-344-01), TLC wise starting material was absent. Workup: The reaction mixture was diluted with ethyl acetate (3000 mL) and washed with water(2000 ml), the organic layer was separated and the aqueous phase was again extracted with ethyl acetate (1×500 mL). The combined organic layer washed with brine(lx 800 mL). The combined organic layer, dried (Na₂SO₄), filtered, and concentrated under reduced pressure to obtained (235 g) as a pale brown solid. The product (235 g) was recrystallized from 10% ethyl acetate/petroleum ether (500 mL), stirred, filtered, and dried to afford mesitylenesulphinamid (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d6) δ 7.01-6.68 (m, 2H), 6.23-5.77 (m, 2H), 2.52-2.50 (m, 6H), 2.32-1.93 (m, 3H) and Peak-2 (desired): (S)-2,4,6-trimethylbenzenesulfinamide (51.6 g, 267 mmol, 35.0% yield) as a white colour solid. ¹H NMR (400 MHz, DMSO-d6) δ 6.87 (s, 2H), 6.16-5.82 (m, 2H), 2.53-2.50 (m, 6H), 2.34-1.93 (m, 3H).

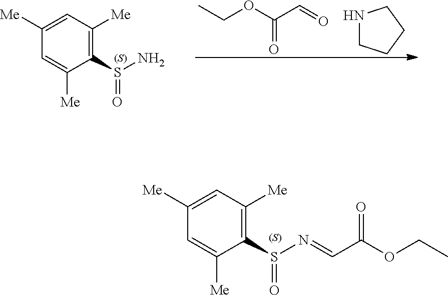

### Step 3:

The compound was synthesized using similar procedure described in reference: To a well stirred solution of (S)-2,4,6-trimethylbenzenesulfinamide (15.5 g, 85 mmol) in dichloromethane (235 mL) and 4 A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction was mass was filtered throw the diatomaceous earth (Celite®) and the bed was washed with DCM. The solvents were removed under reduced pressure to obtained the crude (55 g) as a brownish color mass. The crude compound was purified by ISCO (Column size: 300 g silica column. Adsorbent: 60-120 silica mesh, Mobile phase: 40% EtOAc/Pet ether) and the product was collected at 15-20% of EtOAc. The fractions were concentrated to obtain ethyl (S,E)-2-((mesitylsulfinyl)imino)acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

### Step 4:

General procedure for the synthesis of TCNHPI redox-active esters as in reference ACIE:TCNHPI esters were prepared according to the previously reported general procedure (ACIE paper and references therein): A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of diatomaceous earth (Celite®), SiO₂, or frit funnel) and insed with additional CH₂Cl₂/Et₂O. The solvent was removed under reduced pressure, and purification of the crude mixture by column chromatography afforded the desired TCNHPI redox-active ester. If necessary, the TCNHPI redox-active ester could be further recrystallized from CH₂Cl₂/MeOH.

### Step 5:

4,5,6,7-tetrachloro-1,3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et₂O) afforded 2.15 g (84%) of the title compound. ¹H NMR (400 MHz, CDCl₃): δ 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂₀Cl₄N₂NaO₆ [M+Na]⁺: 534.9968, found: 534.9973.

### Step 6:

Ethyl (S)-5-((tert-butoxycarbonyl)amino)-2-(((S)-mesitylsulfinyl)amino)-3,3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)2·4H2O (0.25 mmol, 25 mol %), Zinc (3 mmol, 3 equiv). The tube was then evacuated and backfilled with argon (three times). Anhydrous NMP (5.0 mL, 0.2 M) was added using a syringe. The mixture was stirred overnight at rt. Then, the reaction mixture was diluted with EtOAc, washed with water,brine and dried over MgSO₄. Upon filtration, the organic layer was concentrated under reduced pressure (water bath at 30° C.), and purified by flash column chromatography (silica gel) to provide the product. Purification by column (2:1 hexanes:EtOAc) afforded 327.6 mg (72%) of the title compound ethyl (S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5, 61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

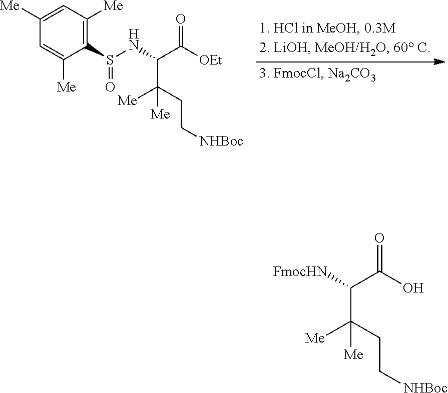

### Step 7:

2-((((9H-fluoren-9-yl)methoxy)carbonyl)amino)-5-((tert-butoxycarbonyl)amino)-3,3-dimethylpentanoic acid: A culture tube was charged with ethyl (S)-5-((tert-butoxycarbonyl)amino)-2-(((S)-mesitylsulfinyl)amino)-3,3-dimethylpentanoate (0.5 mmol, 1.0 equiv), HCl (4.0 equiv) in MeOH (0.3 M) was added via syringe and the resulting mixture was stirred at RT for ca. 10 min (screened by TLC). After the reaction, Et3N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to rt. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes:EtOAc) to afford the product ethyl (S)-5-((tert-butoxycarbonyl)amino)-2-(((S)-mesitylsulfinyl)amino)-3,3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of (S)-2-((((9H-fluoren-9-yl)methoxy)carbonyl)amino)-3-(4,4-difluorocyclohexyl)propanoic Acid

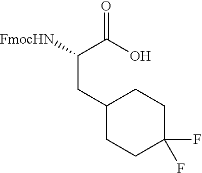

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired (S)-2-((((9H-fluoren-9-yl)methoxy)carbonyl)amino)-3-(4,4-difluorocyclohexyl)propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)-5-(tert-butoxy)-2-({[(9H-fluoren-9-yl)methoxy]carbonyl}amino)-3,3-dimethyl-5-oxopentanoic Acid

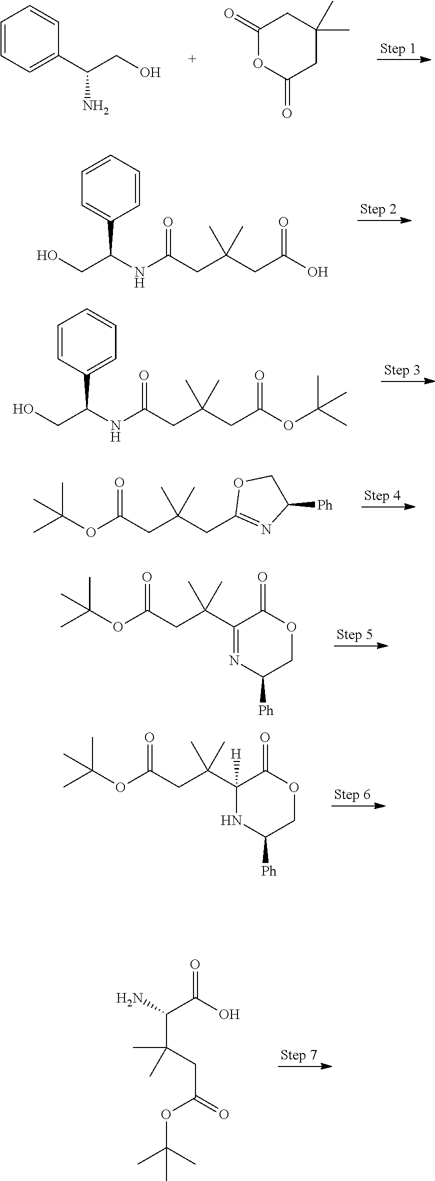

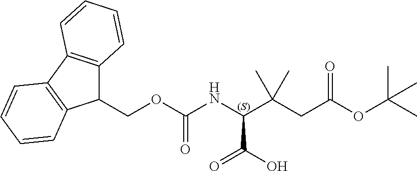

### Step 1:

A solution of 4,4-dimethyldihydro-2H-pyran-2,6(3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d6) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

### Step 2:

(R)-5-((2-Hydroxy-1-phenylethyl)amino)-3,3-dimethyl-5-oxopentanoic acid (12 g, 43.0 mmol) was dissolved in a solution of benzyltrimethylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂/MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LC/MS Condition M: 1.96 min, 336.3 [M+H]⁺. ¹H NMR (300 MHz, DMSO-d6) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

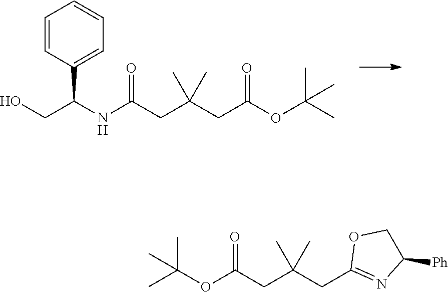

### Step 3:

tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1:5) to give tert-butyl (R)-3,3-dimethyl-4-(4-phenyl-4,5-dihydrooxazol-2-yl)butanoate (5.6 g, 17.64 mmol, 99% yield). ESI-MS(+) m/z: 318.3 [M+H]⁺. ¹H NMR (300 MHz, DMSO-d6) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

### Step 4:

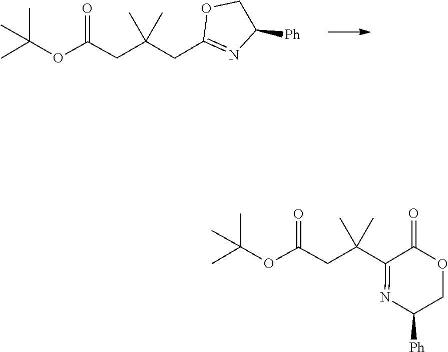 [SMILES: CC(C)(CC(=O)OC(C)(C)C)CC1=N[C@H](c2ccccc2)CO1 ; CC(C)(C)OC(=O)CC(C)(C)C1=N[C@H](c2ccccc2)COC1=O]

A solution of tert-butyl (R)-3,3-dimethyl-4-(4-phenyl-4,5-dihydrooxazol-2-yl)butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl)butanoate (1.3 g, 3.92 mmol, 22.23% yield) as a colorless liquid. ESI-MS(+) m/z: 332.2 [M+H]⁺. ¹H NMR (CDCl₃) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). ¹³C NMR (CD3Cl) δ 26.40, 27.29, 28.00, 40.84, 45.94, 59.72, 70.88, 80.63, 127.13, 127.92, 128.65, 137.58, 155.07, 167.46, 171.95.

### Step 5:

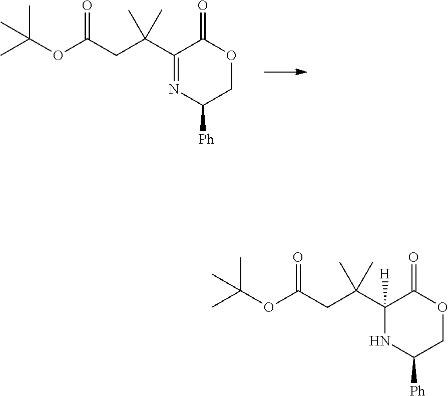 [SMILES: CC(C)(C)OC(=O)CC(C)(C)C1=N[C@H](c2ccccc2)COC1=O ; CC(C)(C)OC(=O)CC(C)(C)[C@@H]1N[C@H](c2ccccc2)COC1=O]

Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl)butanoate (1.3 g, 3.92 mmol) in methanol (50 mL). The reaction flask was purged with H2 (3×) and stirred under H2 for 24 h. After venting the vessel, the reaction mixture was filtered through diatomaceous earth (Celite®), and the filtrate was washed with EtOAc. The crude product was concentrated under vacuo and purified by flash column chromatography on silica gel (EtOAc/Hexanes, 1:8) to give tert-butyl 3-methyl-3-((3S,5R)-2-oxo-5-phenylmorpholin-3-yl)butanoate (1.2 g, 3.33 mmol, 85% yield). ¹HNMR (300 MHz, DMSO-d6) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

### Step 6:

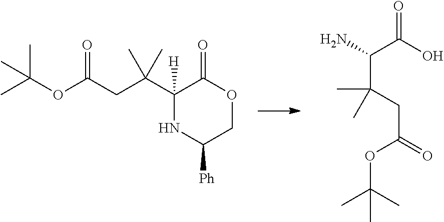 [SMILES: CC(C)(C)OC(=O)CC(C)(C)[C@@H]1N[C@H](c2ccccc2)COC1=O ; CC(C)(C)OC(=O)CC(C)(C)[C@H](N)C(=O)O]

Pearlman's catalyst Pd(OH)₂ on carbon (1.264 g, 1.799 mmol, 20% w/w) was added to a solution of tert-butyl 3-methyl-3-((3S,5R)-2-oxo-5-phenylmorpholin-3-yl)butanoate (1.2 g, 3.60 mmol) in methanol (50 mL)/water (3.13 mL)/TFA (0.625 mL) (40:2.5:0.5, v/v/v). The vessel was purged with H2 and stirred under H2 for 24 h. After venting the vessel, the reaction mixture was filtered through diatomaceous earth (Celite®), and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

### Step 7:

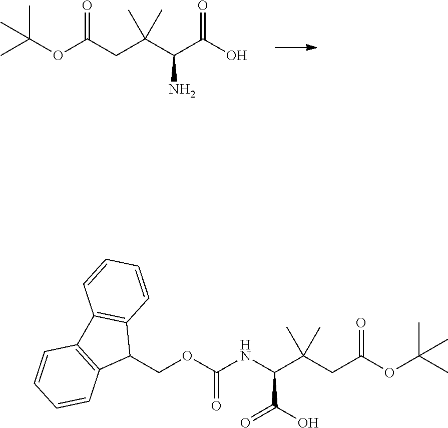 [SMILES: CC(C)(C)OC(=O)CC(C)(C)[C@H](N)C(=O)O ; CC(C)(C)OC(=O)CC(C)(C)[C@H](NC(=O)OCC1c2ccccc2-c2ccccc21)C(=O)O]

The crude product (S)-2-amino-5-(tert-butoxy)-3,3-dimethyl-5-oxopentanoic acid (1 g, 4.32 mmol) dissolved in water (30 mL). Na₂CO₃ (0.916 g, 8.65 mmol) was then added to the above solution. To this solution, fmoc n-hydroxysuccinimide ester (1.458 g, 4.32 mmol) in dioxane (30 mL) was added drop wise at 0° C. and stirred at room temperature for 16 h. The reaction mixture was acidified to pH ˜2 by 1N HCl and extracted with EtOAc (50 mL×3), dried over Na₂SO₄, concentrated under vacuo and purified by flash column chromatography on silica gel (EtOAc/petroleum ether, 35 to 39%) to give (S)-2-((((9H-fluoren-9-yl)methoxy)carbonyl)amino)-5-(tert-butoxy)-3,3-dimethyl-5-oxopentanoic acid (0.73 g, 1.567 mmol, 36.2% yield) as a white solid. LCMS, Analytical LC/MS Condition E, MS (ESI) t_{R}=2.135 min, m/z 452.2 [M−H]⁻. ¹H NMR (400 MHz, DMSO-d6) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl)methoxy]carbonyl}amino)-3-(morpholin-4-yl)propanoic Acid

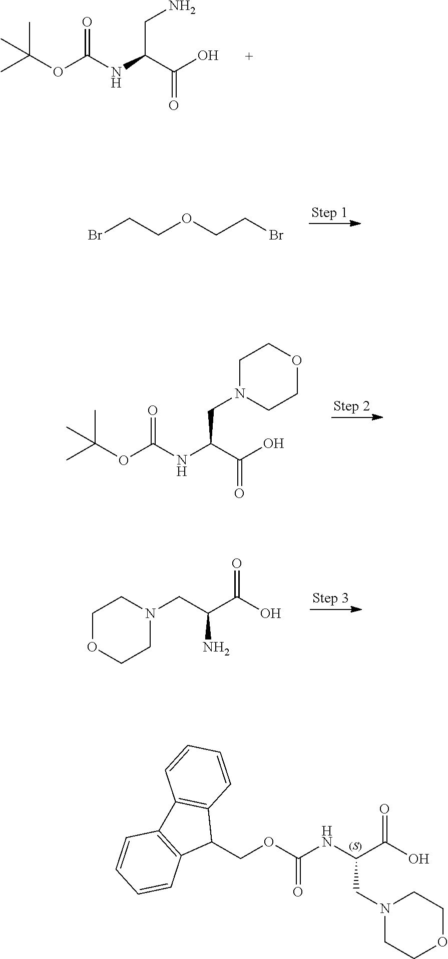

### Step 1:

In a 2-L multi-necked round-bottomed flask fitted with a thermo pocket was added (S)-3-amino-2-((tert-butoxycarbonyl)amino)propanoic acid (50 g, 245 mmol), dioxane (500 mL), followed by 1-bromo-2-(2-bromoethoxy)ethane (30.8 mL, 245 mmol) at rt. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to get light red solution which was acidified to pH 3. The resulting mixture was concentrated under high vacuum pump (˜4 mbar) at 60° C. to get (S)-2-((tert-butoxycarbonyl)amino)-3-morpholinopropanoic acid (67 g, 244 mmol, 100% yield) pale yellow solid. Analytical LC/MS Condition M: 0.56 min, 275.2 [M+H]⁺.

### Step 2:

To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at rt for 12 h. The volatile was evaporated to get pale yellow sticky crude (S)-2-amino-3-morpholinopropanoic acid (16 g, 92 mmol, 97% yield) This crude was taken for next step without further purification. MS (ESI) m/z 175.2 [M+H]⁺.

### Step 3:

The crude product (S)-2-amino-3-morpholinopropanoic acid (11 g, 63.1 mmol) dissolved in water (250 mL). Na₂CO₃ (13.39 g, 126 mmol) was then added to the above solution. To this solution, Fmoc N-hydroxysuccinimide ester (21.30 g, 63.1 mmol) was added dropwise at 0 C and stirred at room temperature for 16 h. The reaction mixture was acidified to pH ˜2 by 1N HCl and extracted with EtOAc (500 mL×3), dried over Na₂SO₄, concentrated under vacuo, and purified by flash column chromatography on silica gel (petroleum ether/EtOAc, 0-100% then MeOH/CHCl₃ 0-15%) to get (S)-2-((((9H-fluoren-9-yl)methoxy)carbonyl)amino)-3-morpholinopropanoic acid (23 g, 55.9 mmol, 89% yield) as a brown solid. Analytical LC/MS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy)carbonyl]amino}-2-({[(9H-fluoren-9-yl)methoxy]carbonyl}amino)butanoic Acid

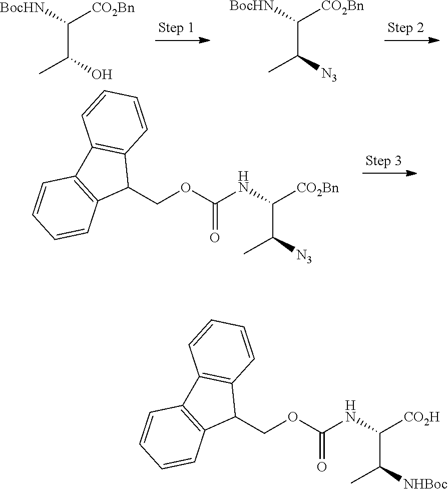

### Step 1:

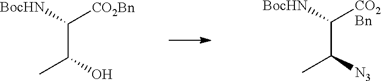 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1]

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex:EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex:EtOAc 95:5 a 9:1) to give benzyl (2S,3S)-3-azido-2-((tert-butoxycarbonyl)amino)butanoate (20 g, 59.8 mmol, 84% yield) as colorless liquid. Analytical LC/MS Condition E: 3.13 min, 333.2 [M−H]⁻.

### Step 2:

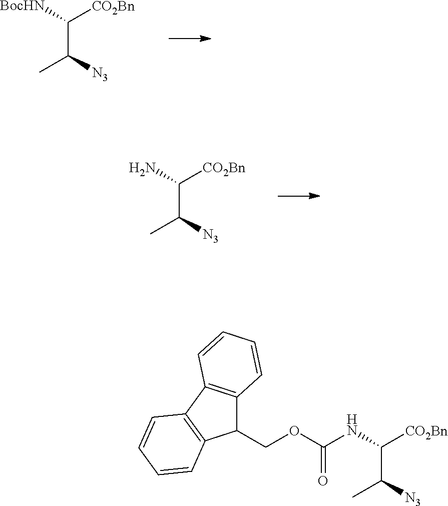 [SMILES: C[C@H](N=[N+]=[N-])[C@H](NC(=O)OC(C)(C)C)C(=O)OCc1ccccc1 ; C[C@H](N=[N+]=[N-])[C@H](N)C(=O)OCc1ccccc1 ; C[C@H](N=[N+]=[N-])[C@H](NC(=O)OCC1c2ccccc2-c2ccccc21)C(=O)OCc1ccccc1]

A solution of benzyl (2S,3S)-3-azido-2-((tert-butoxycarbonyl)amino)butanoate (20 g, 59.8 mmol), dichloromethane (300 mL) and TFA (50 mL, 649 mmol) was stirred for 2 h at 23° C. and then evaporated to dryness to give the corresponding amine. The above amine was redisolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to rt and stirred for 3 h. It was extracted with EtOAc and washed with 0.5 M HCl solution and then brine solution. It was concentrated to get crude liquid. The above crude was purified by silica gel column chromatography. The product was eluted at 20% EtOAc in petroleum ether. The fractions were concentrated to get benzyl (2S,3S)-2-((((9H-fluoren-9-yl)methoxy)carbonyl)amino)-3-azidobutanoate (23 g, 50.4 mmol, 84% yield) as a colorless liquid. Analytical LC/MS Condition E: 3.70 min, 479.3 [M+Na]⁺.

### Step 3:

To a multi-neck round-bottled flask was charged benzyl (2S,3S)-2-((((9H-fluoren-9-yl)methoxy)carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirring under nitrogen for 12 h. The reaction mass was filtered through cellite bed, washed the bed with THF/Water mixture. The mother liquid was concentrated and washed with EtOAc. Then pH of water layer was adjusted to 7-6 using 1.5 N HCl solution. The resulting white solid was extracted with ethylacetate. The above reaction was repeated three more times. The combined organics were washed with water and brine solution, dried over sodium sulphate, and concentrated to afford (2S,3S)-2-((((9H-fluoren-9-yl)methoxy)carbonyl)amino)-3-((tert-butoxycarbonyl)amino)butanoic acid as a white solid (28 g). This was mixed with a previously obtained batch (8 g) in DCM (200 mL). n-Hexane (IL) was added to the above solution and sonicated for 2 min. The solids were filtered, rinsed with hexanes and dried overnight to give (2S,3S)-2-((((9H-fluoren-9-yl)methoxy)carbonyl)amino)-3-((tert-butoxycarbonyl)amino)butanoic acid (36 g, 81 mmol, 92% yield) as a white powder. Analytical LC/MS Condition E: 1.90 min, 439.2 [M−H]⁻. ¹H NMR (400 MHz, DMSO-d6) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). 13C NMR (101 Hz, DMSO-d6) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of (S)-2-((((9H-fluoren-9-yl)methoxy)carbonyl)amino)-2-(1-(((tert-butoxycarbonyl)amino)methyl)cyclopropyl)acetic Acid

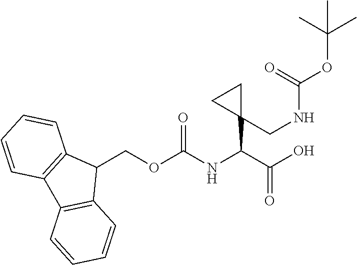 [SMILES: CC(C)(C)OC(=O)NCC1([C@H](NC(=O)OCC2c3ccccc3-c3ccccc32)C(=O)O)CC1]

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired product (0.65 g, 22% yield) as a white solid after purification by flash column chromatography (Red Sep, 40 g, SiO₂, 35 to 40% EtOAc:hexanes (compound ELSD active)). Analytical LC/MS Condition E: 2.04 min, 465.2 [M−H]⁻. ¹H NMR (300 MHz, DMSO-d6) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of (S)-2-((((9H-fluoren-9-yl)methoxy)carbonyl)amino)-2-(1-(tert-butoxycarbonyl)azetidin-3-yl)acetic Acid

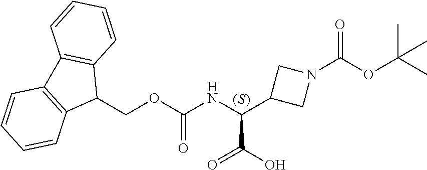

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E: 1.87 min, 467.2 [M−H]⁻. ¹H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

### Preparation of Example 1000

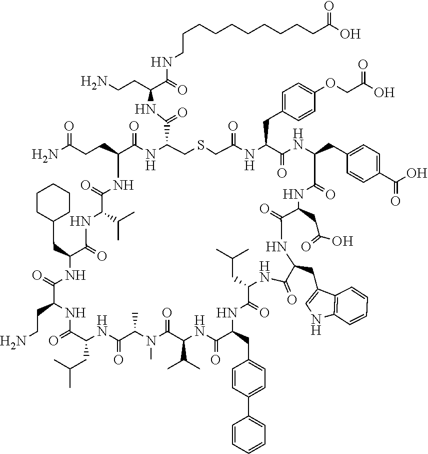 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

To a 45-mL polypropylene solid-phase reaction vessel was added 2-chlorotrityl resin pre-loaded with 11-((((9H-fluoren-9-yl)methoxy)carbonyl)amino)undecanoic acid on a 50 μmol scale, and the reaction vessel was placed on the Symphony X peptide synthesizer. The following procedures were then performed sequentially:
“Symphony X Resin-swelling procedure” was followed;
“Symphony X Single-coupling procedure” was followed with Fmoc-Dab(Boc)-OH;
“Symphony X Single-coupling procedure” was followed with Fmoc-Cys(Trt)-OH;
“Symphony X Single-coupling procedure” was followed with Fmoc-Glu(OtBu)-OH;
“Symphony X Single-coupling procedure” was followed with Fmoc-Val-OH;
“Symphony X Single-coupling procedure” was followed with Fmoc-Cha-OH;
“Symphony X Single-coupling procedure” was followed with Fmoc-Dab(OtBu)-OH;
“Symphony X Single-coupling procedure” or “Symphony Double-coupling procedure” was followed with Fmoc-D-Leu-OH;
“Symphony X Single-coupling procedure” or “Symphony Double-coupling procedure” was followed with Fmoc-NMe-Ala-OH;
“Symphony X Single-coupling procedure” or “Symphony Double-coupling procedure” was followed with Fmoc-Val-OH;
“Symphony X double-coupling procedure” was followed with Fmoc-Bip-OH;
“Symphony X single-coupling procedure” was followed with Fmoc-Leu-OH;
“Symphony X single-coupling procedure” was followed with Fmoc-Trp(Boc)-OH;
“Symphony X single-coupling procedure” was followed with Fmoc-Asp(tBu)-OH;
“Symphony Single-coupling procedure” was followed with Fmoc-Phe(4-COOtBu)OH;
“Symphony X Single-coupling procedure” was followed with Fmoc-Tyr(CH₂COOtBu)-OH;
“Symphony X Chloroacetic Anhydride coupling procedure” was followed;
“Global Deprotection Method A” was followed;
“Cyclization Method” was followed.

The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 8% B, 8-48% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.4 mg, and its estimated purity by LCMS analysis was 95%.
Analysis condition A: Retention time=1.44 min; ESI-MS(+) m/z [M+2H]²⁺: 1098.1.
Analysis condition B: Retention time=1.94 min; ESI-MS(+) m/z [M+2H]²⁺: 1097.9.

### Preparation of Example 1001

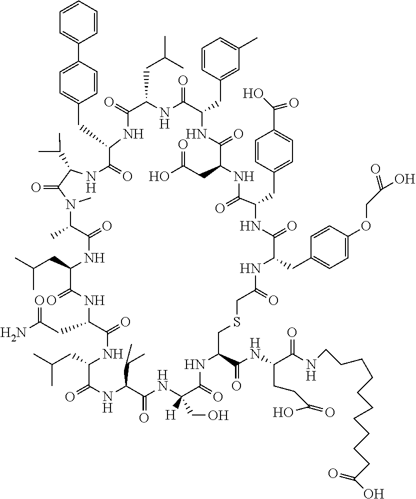 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCC(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

To a 45-mL polypropylene solid-phase reaction vessel was added 2-chlorotrityl resin pre-loaded with 11-((((9H-fluoren-9-yl)methoxy)carbonyl)amino)undecanoic acid on a 50 μmol scale, and the reaction vessel was placed on the Symphony peptide synthesizer. The following procedures were then performed sequentially:
“Symphony Resin-swelling procedure” was followed;
“Symphony Single-coupling procedure” was followed with Fmoc-Asp(tBu)-OH;
“Symphony Single-coupling procedure” was followed with Fmoc-Cys(Trt)-OH;
“Symphony Single-coupling procedure” was followed with Fmoc-Ser(tBu)-OH;
“Symphony Single-coupling procedure” was followed with Fmoc-Val-OH;
“Symphony Single-coupling procedure” was followed with Fmoc-Leu-OH;
“Symphony Single-coupling procedure” was followed with Fmoc-Asn(Trt)-OH;
“Symphony Single-coupling procedure” or “Symphony Double-coupling procedure” was followed with Fmoc-D-Leu-OH;
“Symphony Single-coupling procedure” or “Symphony Double-coupling procedure” was followed with Fmoc-NMe-Ala-OH;
“Symphony Single-coupling procedure” or “Symphony Double-coupling procedure” was followed with Fmoc-Val-OH;
“Symphony double-coupling procedure” was followed with Fmoc-Bip-OH;
“Symphony single-coupling procedure” was followed with Fmoc-Leu-OH;
“Symphony single-coupling procedure” was followed with Fmoc-Phe(3-Me)-OH;
“Symphony single-coupling procedure” was followed with Fmoc-Asp(tBu)-OH;
“Symphony Single-coupling procedure” was followed with Fmoc-Phe(4-COOtBu)OH;
“Symphony Single-coupling procedure” was followed with Fmoc-Tyr(CH₂COOtBu)-OH;
“Symphony Chloroacetic Anhydride coupling procedure” was followed;
“Global Deprotection Method A” was followed;
“Cyclization Method” was followed.

The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 8% B, 8-48% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.4 mg, and its estimated purity by LCMS analysis was 95%.

Analysis condition A: Retention time=1.44 min; ESI-MS(+) m/z [M+2H]²⁺: 1098.1.

Analysis condition B: Retention time=1.94 min; ESI-MS(+) m/z [M+2H]²⁺: 1097.9.

### Preparation of Example 1002

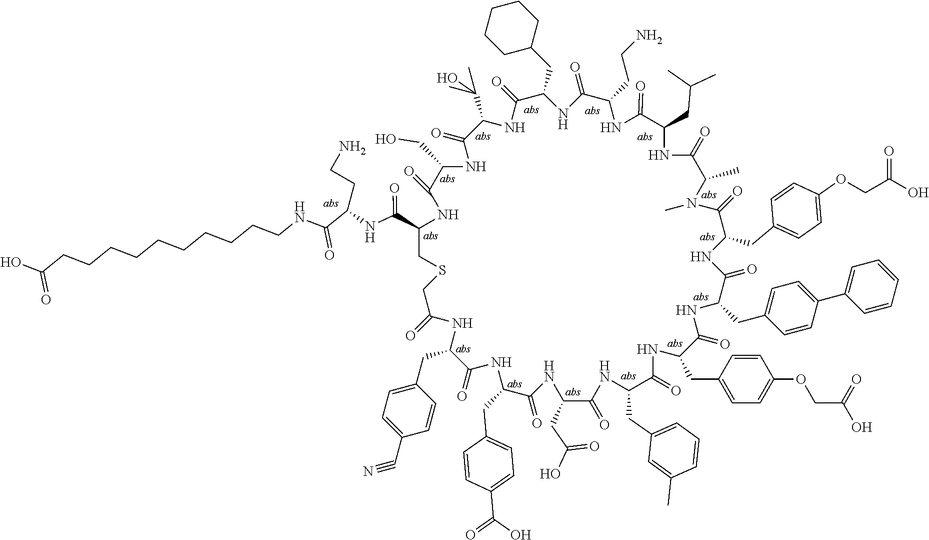 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(C#N)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

To a 45-mL polypropylene solid-phase reaction vessel was added 2-Chlorotrityl resin pre-loaded with 11-((((9H-fluoren-9-yl)methoxy)carbonyl)amino)undecanoic acid (180 mg, 0.100 mmol), and the reaction vessel was placed on the Prelude peptide synthesizer. The following procedures were then performed sequentially:
“Prelude Resin-swelling procedure” was followed;
“Prelude Single-coupling procedure” was followed with Fmoc-Dab(Boc)-OH;
“Prelude Single-coupling procedure” was followed with Fmoc-Cys(Trt)-OH;
“Prelude Single-coupling procedure” was followed with Fmoc-Ser(tBu)-OH;
“Prelude Single-coupling procedure” was followed with Fmoc-Val(β-OH)—OH;
“Prelude Single-coupling procedure” was followed with Fmoc-Cha-OH;
“Prelude Single-coupling procedure” was followed with Fmoc-Dab(Boc)-OH;
“Prelude Single-coupling procedure” or “Prelude double-coupling procedure” was followed with Fmoc-D-Leu-OH;
“Prelude Single-coupling procedure” was followed with Fmoc-NMe-Ala-OH;
“Prelude Single-coupling procedure” was followed with Fmoc-Tyr(CH₂COOtBu)-OH;
“Prelude Single-coupling procedure” was followed with Fmoc-Bip-OH;
“Prelude Single-coupling procedure” was followed with Fmoc-Tyr(CH₂COOtBu)-OH;
“Prelude Single-coupling procedure” was followed with Fmoc-Phe(3-Me)-OH;
“Prelude Single-coupling procedure” was followed with Fmoc-Asp(tBu)-OH;
   The resin was split into 0.025 mmol and was transferred to a different 45-mL polypropylene solid-phase reaction vessel, and the reaction vessel was placed on the Symphony X peptide synthesizer. The following procedures were then performed sequentially:
“Symphony X Resin-swelling procedure” was followed;
“Symphony X Single-coupling procedure” was followed with Phe(4-COOtBu)-OH;
“Symphony X Single-Coupling Manual Addition Procedure A” was followed with Phe(4-CN);
“Symphony X Chloroacetic Anhydride coupling procedure” was followed;
“Symphony X Final rinse and dry procedure” was followed;
“Global Deprotection Method A” was followed;
“Cyclization Method A” was followed.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 30% B, 30-70% B over 20 minutes, then a 0-minute hold at 100% B; Flow Rate: 20 mL/min; Column Temperature: 25 C. Fraction collection was triggered by UV signals. Fractions containing the desired product were combined and dried via centrifugal evaporation. The yield of the product was 36.2 mg, and its estimated purity by LCMS analysis was 96.4%. Analysis condition: Retention time=1.54 min; ESI-MS(+) m/z [M+2H]²⁺: 1202.4.

### Preparation of Example 1003

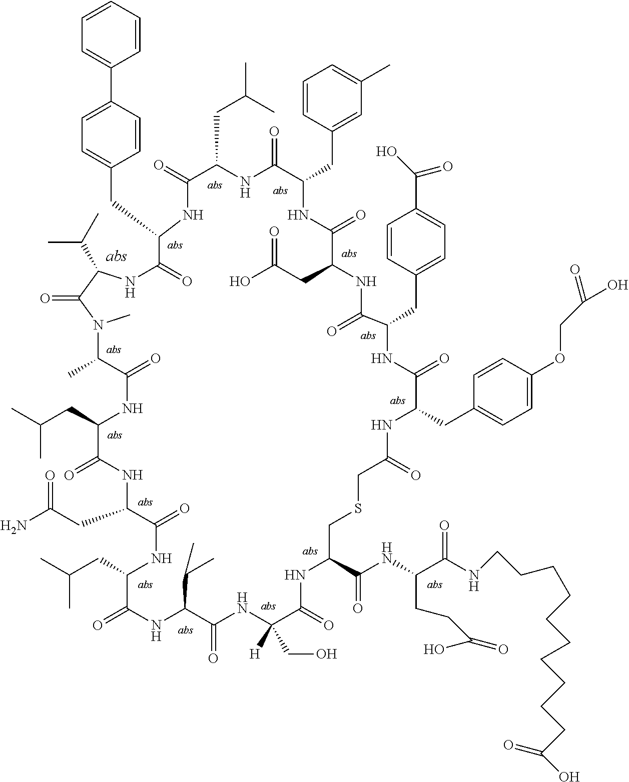 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCC(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1003 was prepared on a 50 μmol scale. The yield of the product was 25.4 mg, and its estimated purity by LCMS analysis was 90.2%. Analysis condition B: Retention time=1.96 min; ESI-MS(+) m/z [M+2H]²⁺: 1105.2.

### Preparation of Example 1004

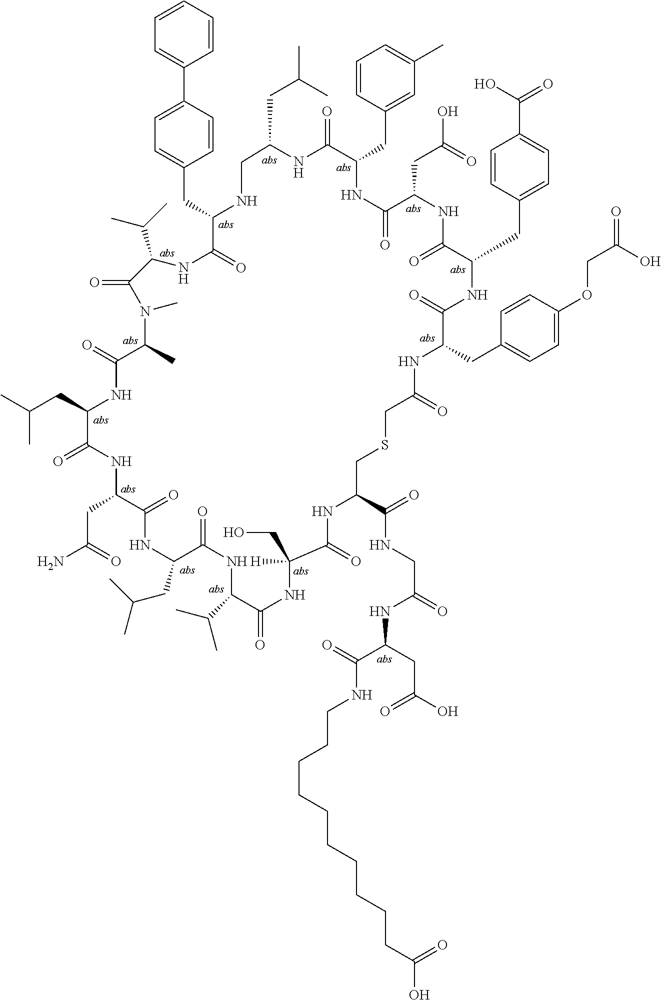 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC[C@H](CC(C)C)NC2=O)c1]

Example 1004 was prepared on a 50 μmol scale. The yield of the product was 35.2 mg, and its estimated purity by LCMS analysis was 92%. Analysis condition A: Retention time=1.49 min; ESI-MS(+) m/z [M+2H]²⁺: 1127.1.

### Preparation of Example 1005

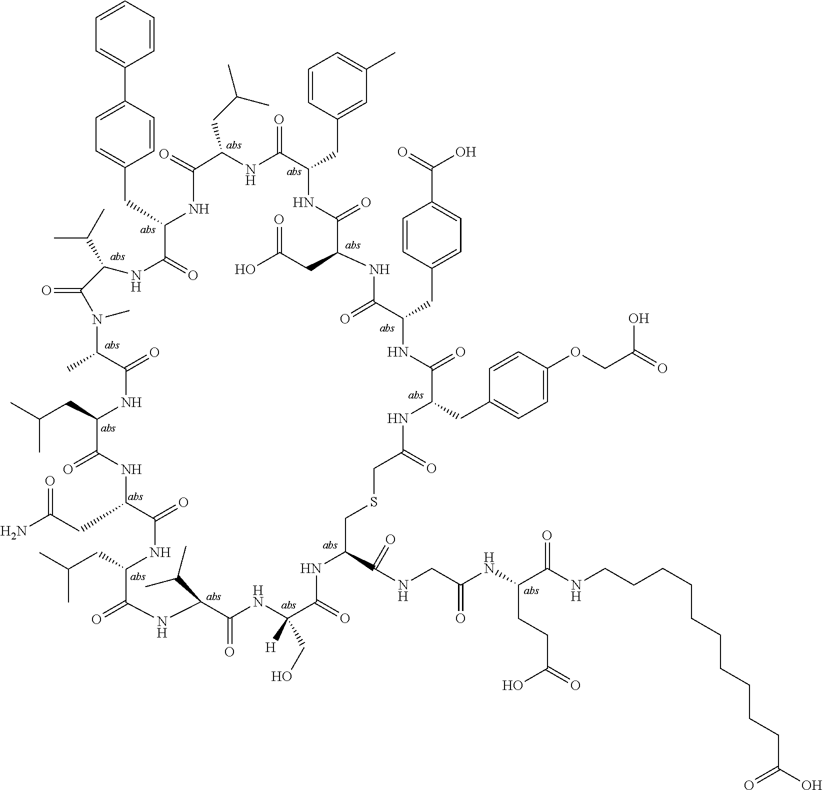 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1005 was prepared on a 50 μmol scale. The yield of the product was 9.2 mg, and its estimated purity by LCMS analysis was 94.4%. Analysis condition A: Retention time=1.33 min; ESI-MS(+) m/z [M+2H]²⁺: 1134.

### Preparation of Example 1006

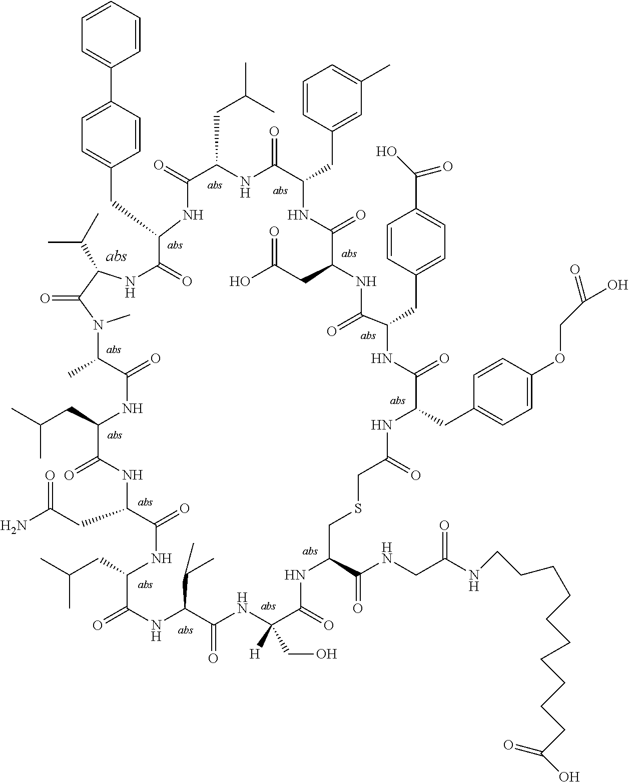 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1006 was prepared on a 50 μmol scale. The yield of the product was 21.2 mg, and its estimated purity by LCMS analysis was 95.7%. Analysis condition A: Retention time=1.58 min; ESI-MS(+) m/z [M+2H]²⁺: 1067.3.

### Preparation of Example 1007

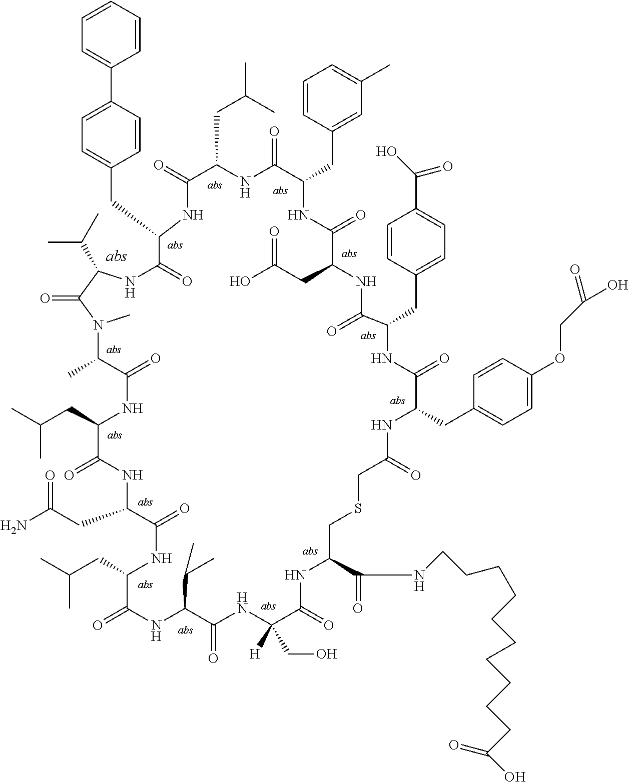 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1007 was prepared on a 50 μmol scale. The yield of the product was 16.8 mg, and its estimated purity by LCMS analysis was 99.1%. Analysis condition A: Retention time=1.64 min; ESI-MS(+) m/z [M+2H]²⁺: 1041.1.

### Preparation of Example 1008

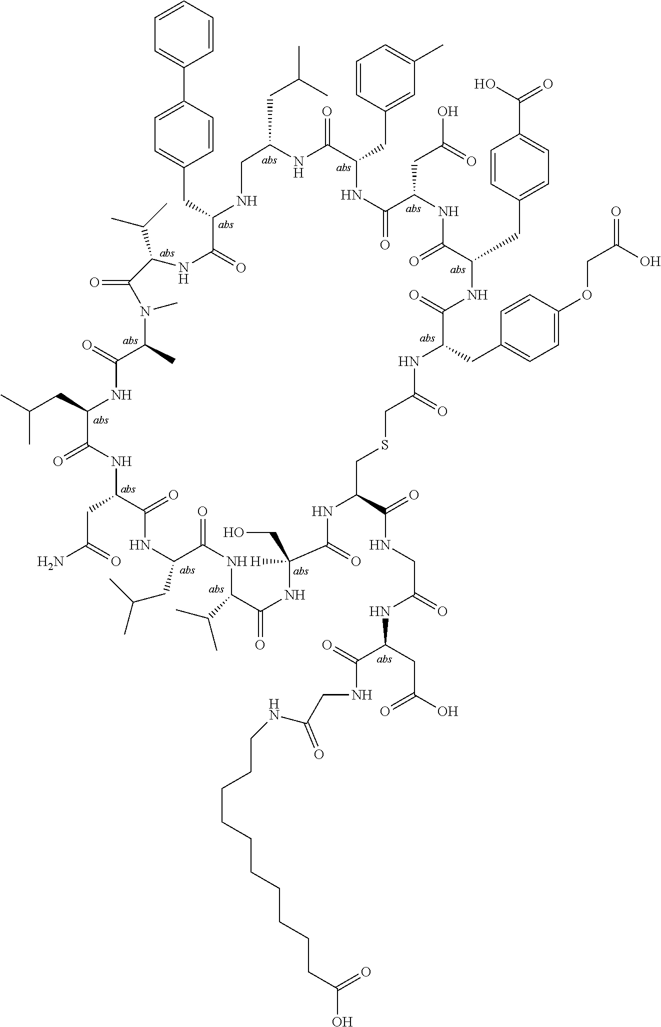 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)O)C(=O)NCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC[C@H](CC(C)C)NC2=O)c1]

Example 1008 was prepared on a 50 μmol scale. The yield of the product was 22.5 mg, and its estimated purity by LCMS analysis was 93.3%. Analysis condition B: Retention time=1.94 min; ESI-MS(+) m/z [M+2H]²⁺: 1155.1.

### Preparation of Example 1009

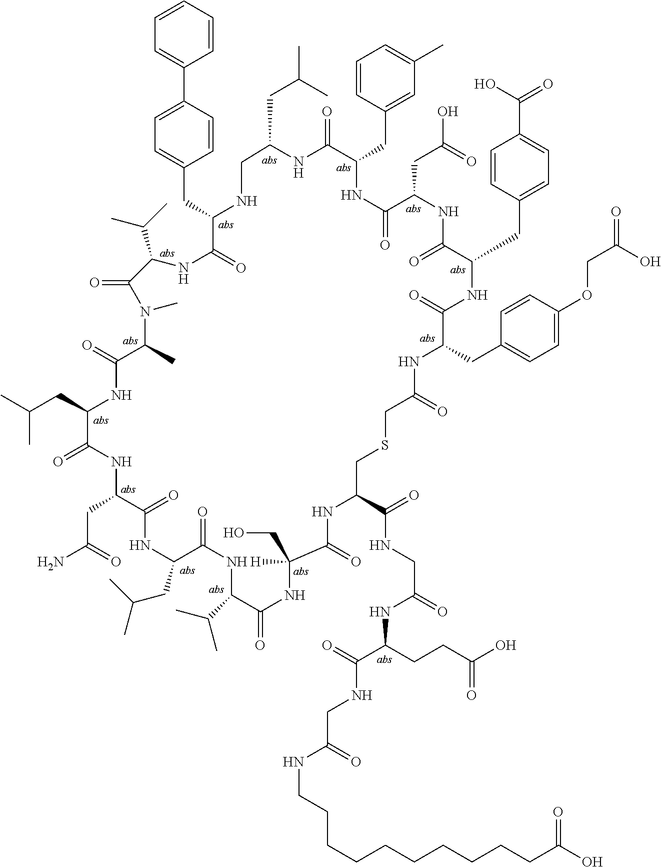 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC[C@H](CC(C)C)NC2=O)c1]

Example 1009 was prepared on a 50 μmol scale. The yield of the product was 13.8 mg, and its estimated purity by LCMS analysis was 98.5%. Analysis condition A: Retention time=1.35 min; ESI-MS(+) m/z [M+2H]²⁺: 1161.9.

### Preparation of Example 1010

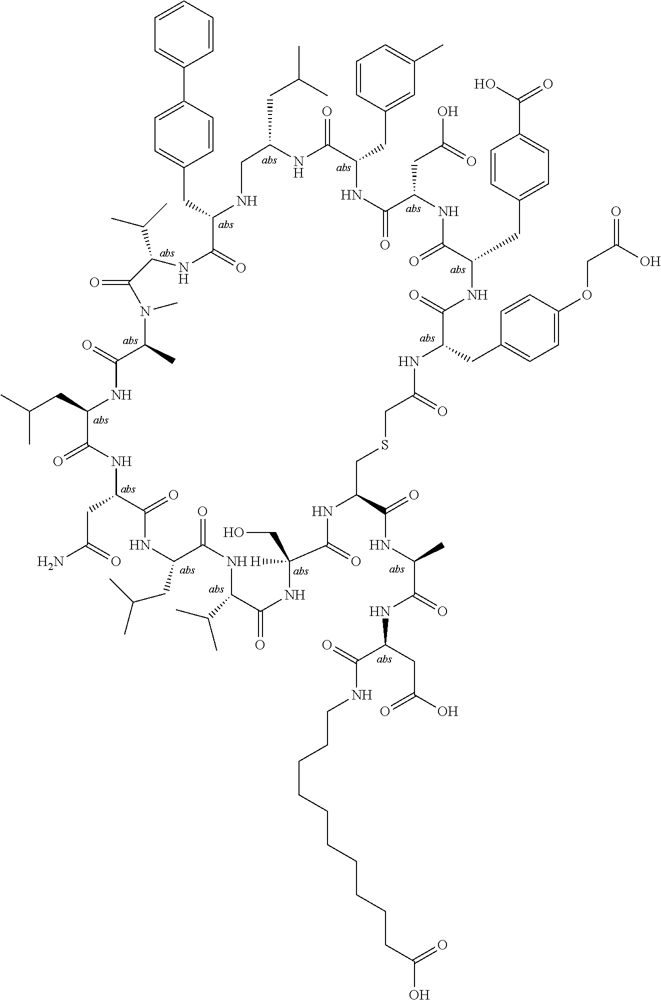 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(=O)N[C@@H](CC(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC[C@H](CC(C)C)NC2=O)c1]

Example 1010 was prepared on a 50 μmol scale. The yield of the product was 23.6 mg, and its estimated purity by LCMS analysis was 98.1%. Analysis condition B: Retention time=1.95 min; ESI-MS(+) m/z [M+2H]²⁺: 1134.

### Preparation of Example 1011

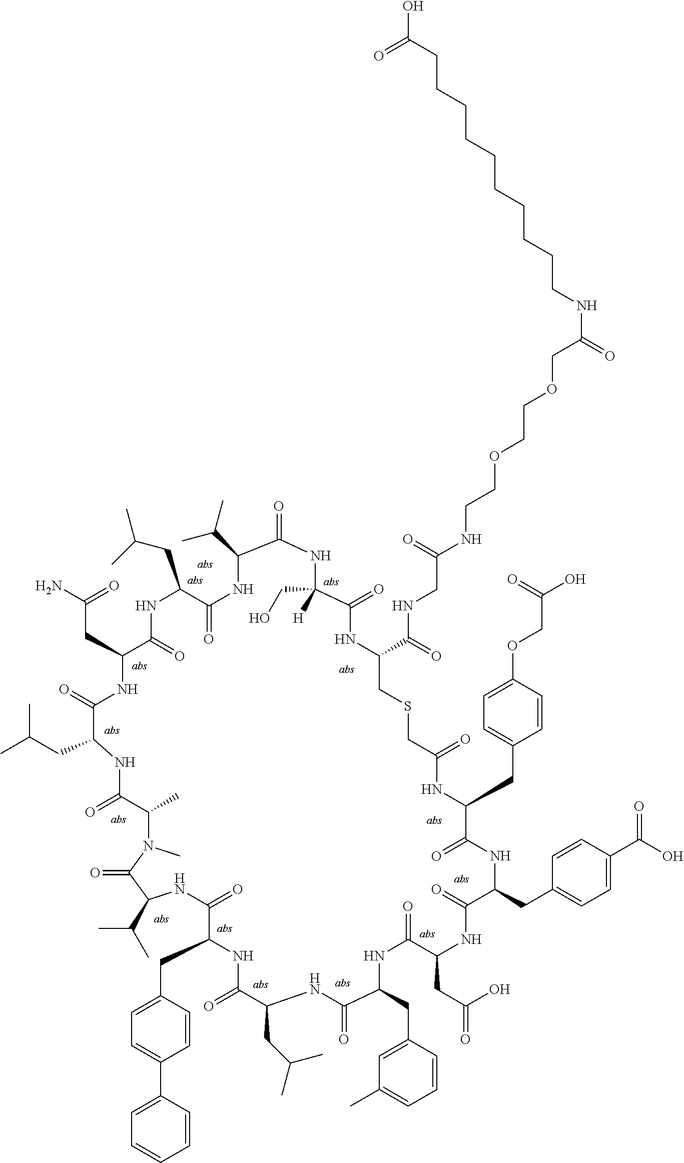

Example 1011 was prepared on a 50 μmol scale. The yield of the product was 38 mg, and its estimated purity by LCMS analysis was 96.2%. Analysis condition A: Retention time=1.41 min; ESI-MS(+) m/z [M+2H]²⁺: 1142.

### Preparation of Example 1012

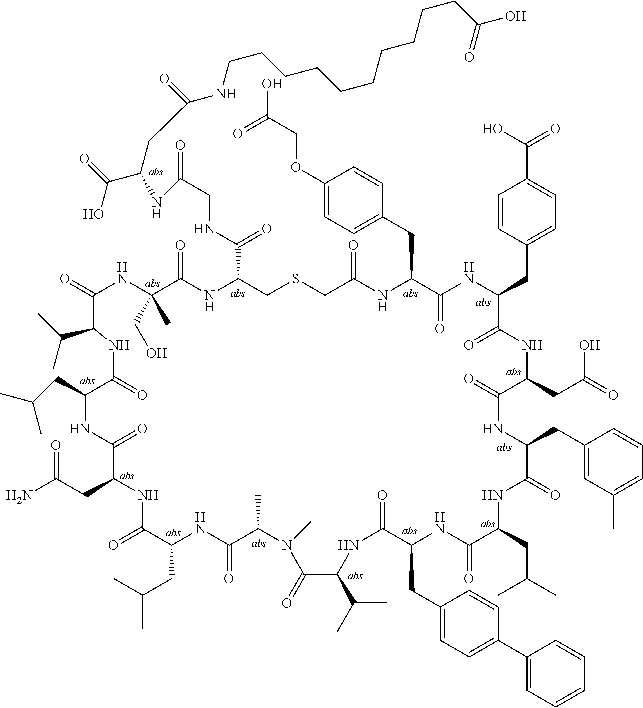 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)NCCCCCCCCCCC(=O)O)C(=O)O)NC(=O)[C@](C)(CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1012 was prepared on a 50 μmol scale. The yield of the product was 12.5 mg, and its estimated purity by LCMS analysis was 93.3%. Analysis condition B: Retention time=1.96 min; ESI-MS(+) m/z [M+2H]²⁺: 1126.9.

### Preparation of Example 1013

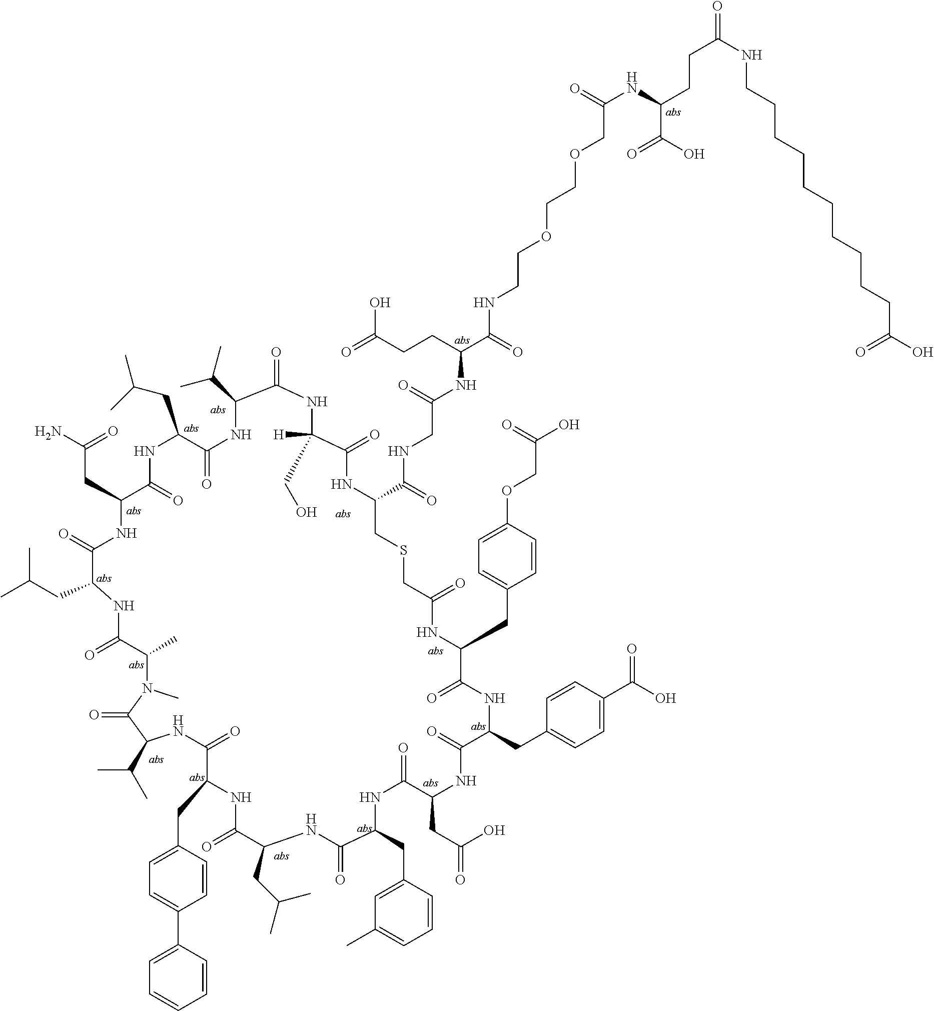

Example 1013 was prepared on a 50 μmol scale. The yield of the product was 19 mg, and its estimated purity by LCMS analysis was 96.7%. Analysis condition A: Retention time=1.28 min; ESI-MS(+) m/z [M+2H]²⁺: 1271.2.

### Preparation of Example 1014

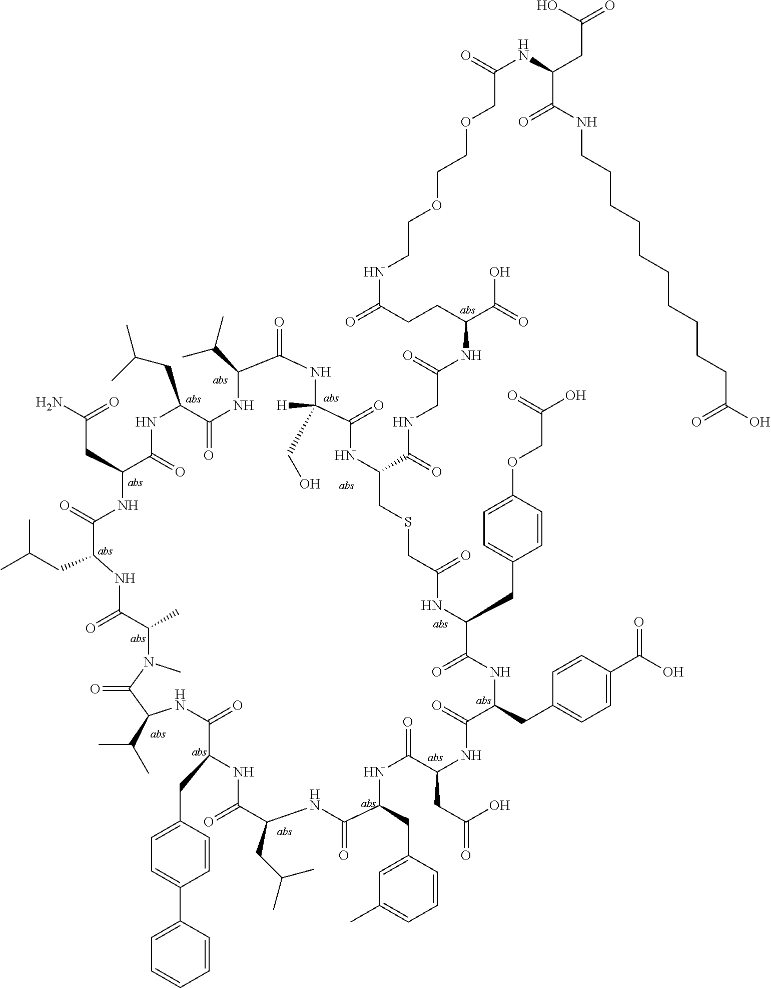

Example 1014 was prepared on a 50 μmol scale. The yield of the product was 38.5 mg, and its estimated purity by LCMS analysis was 90%. Analysis condition A: Retention time=1.44 min; ESI-MS(+) m/z [M+2H]²⁺: 1264.1.

### Preparation of Example 1015

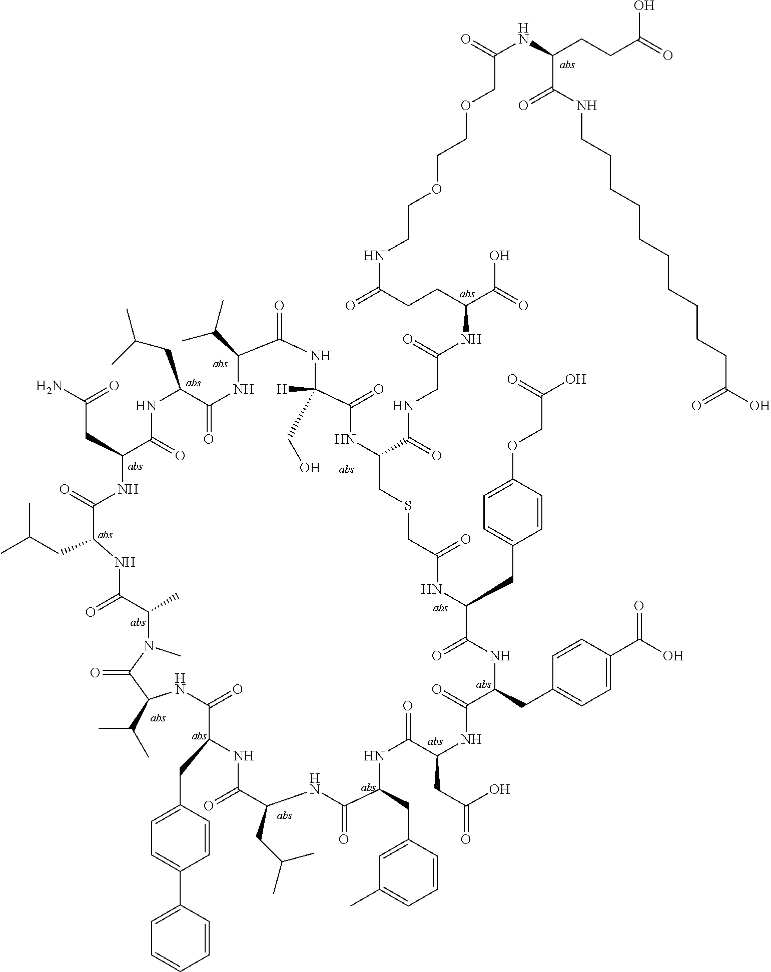

Example 1015 was prepared on a 50 μmol scale. The yield of the product was 38 mg, and its estimated purity by LCMS analysis was 91.6%. Analysis condition A: Retention time=1.29 min; ESI-MS(+) m/z [M+2H]²⁺: 1271.3.

### Preparation of Example 1016

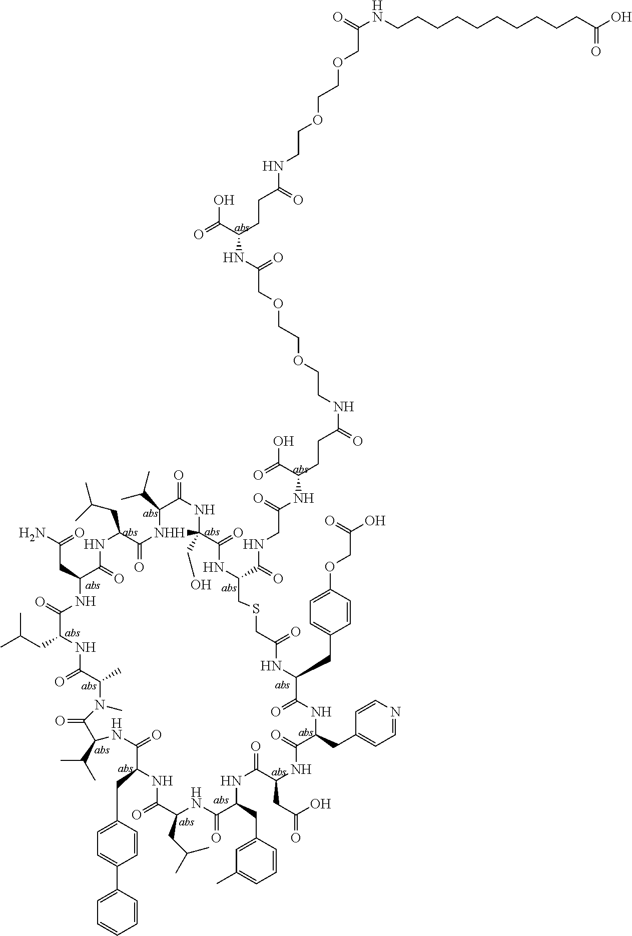 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCC(=O)N[C@@H](CCC(=O)NCCOCCOCC(=O)N[C@@H](CCC(=O)NCCOCCOCC(=O)NCCCCCCCCCCC(=O)O)C(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1016 was prepared on a 50 μmol scale. The yield of the product was 23.2 mg, and its estimated purity by LCMS analysis was 87.1%. Analysis condition B: Retention time=1.95 min; ESI-MS(+) m/z [M+2H]²⁺: 1322.2.

### Preparation of Example 1017

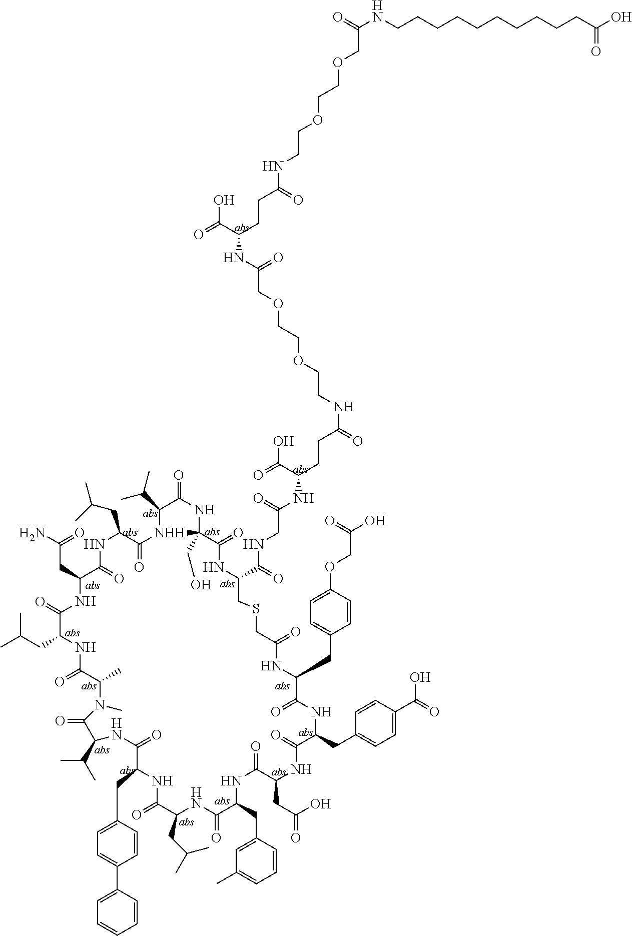 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)NCCOCCOCC(=O)N[C@@H](CCC(=O)NCCOCCOCC(=O)NCCCCCCCCCCC(=O)O)C(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1017 was prepared on a 50 μmol scale. The yield of the product was 15.8 mg, and its estimated purity by LCMS analysis was 99%. Analysis condition A: Retention time=1.41 min; ESI-MS(+) m/z [M+2H]²⁺: 896.1.

### Preparation of Example 1018

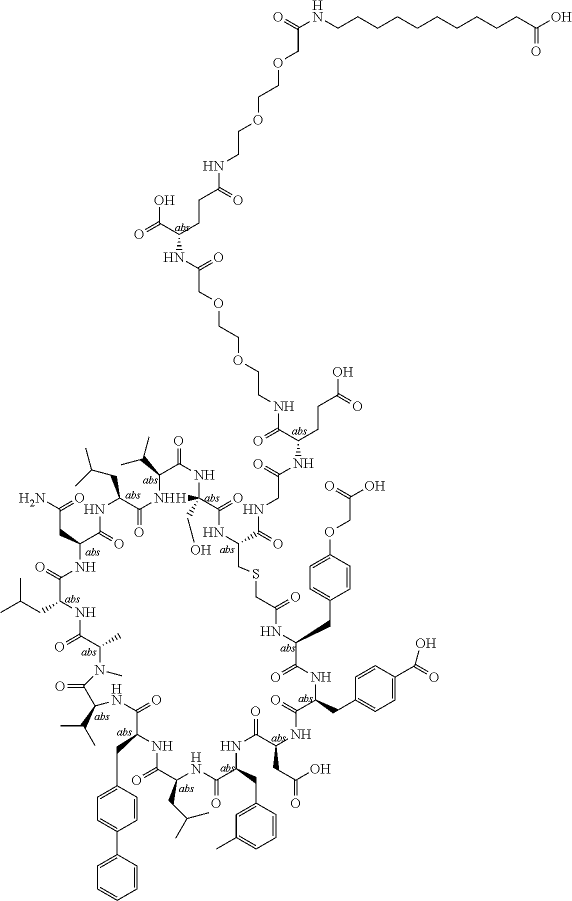 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCCOCCOCC(=O)N[C@@H](CCC(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1018 was prepared on a 50 μmol scale. The yield of the product was 30.6 mg, and its estimated purity by LCMS analysis was 90%. Analysis condition B: Retention time=2.04 min; ESI-MS(+) m/z [M+2H]²⁺: 1343.9.

### Preparation of Example 1019

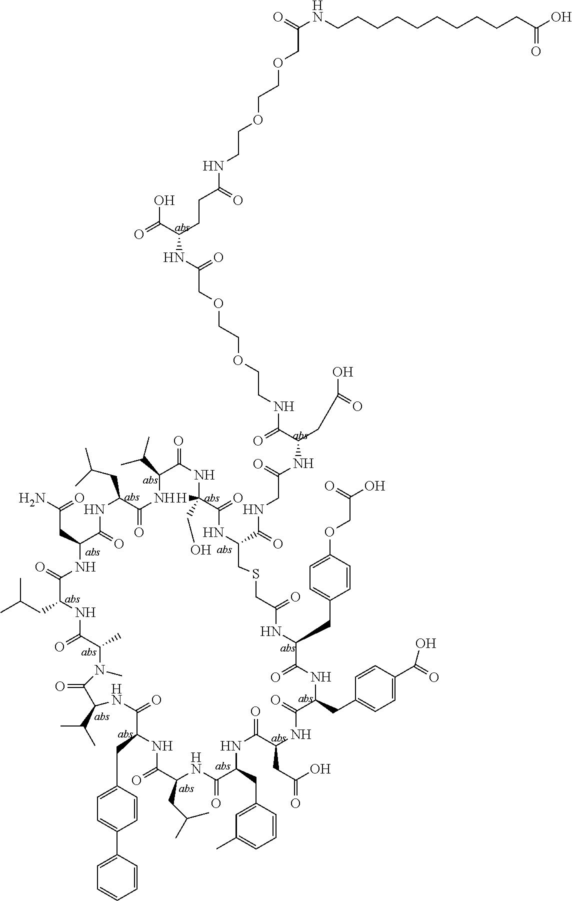 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)O)C(=O)NCCOCCOCC(=O)N[C@@H](CCC(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1019 was prepared on a 50 μmol scale. The yield of the product was 39.3 mg, and its estimated purity by LCMS analysis was 96.1%. Analysis condition A: Retention time=1.45 min; ESI-MS(+) m/z [M+2H]²⁺: 891.4.

### Preparation of Example 1020

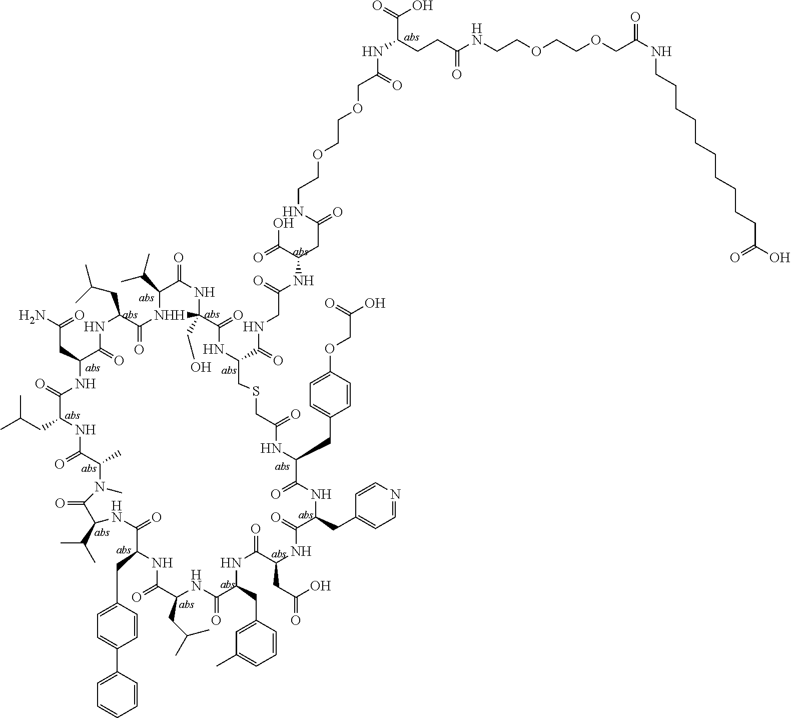 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCC(=O)N[C@@H](CC(=O)NCCOCCOCC(=O)N[C@@H](CCC(=O)NCCOCCOCC(=O)NCCCCCCCCCCC(=O)O)C(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1020 was prepared on a 50 μmol scale. The yield of the product was 28.2 mg, and its estimated purity by LCMS analysis was 90%. Analysis condition B: Retention time=1.99, 2.03 min; ESI-MS(+) m/z [M+2H]²⁺: 891.1.

### Preparation of Example 1021

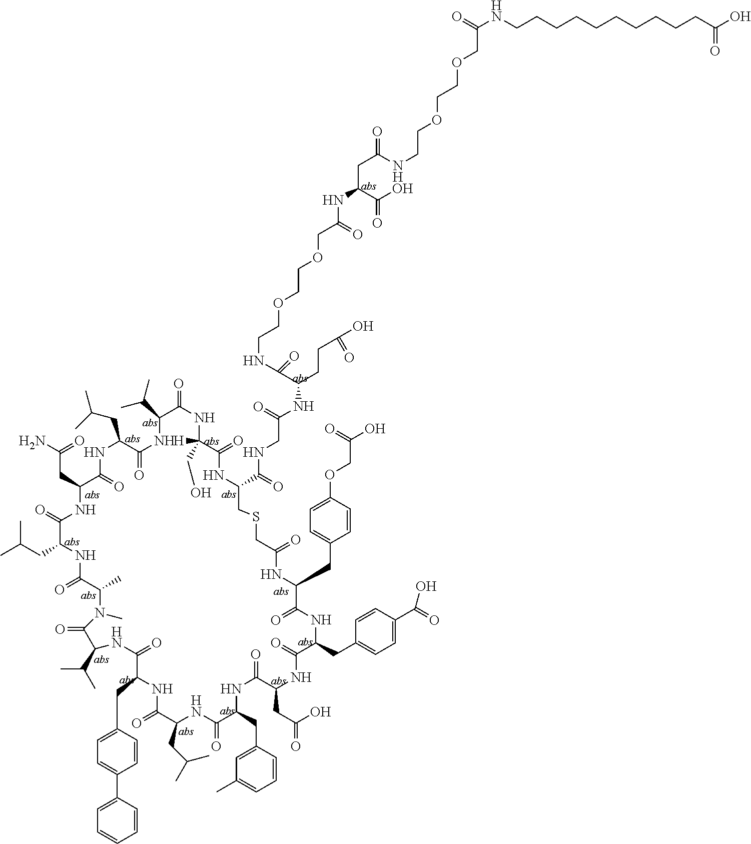 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCCOCCOCC(=O)N[C@@H](CC(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1021 was prepared on a 50 μmol scale. The yield of the product was 11.2 mg, and its estimated purity by LCMS analysis was 91.4%. Analysis condition A: Retention time=1.32 min; ESI-MS(+) m/z [M+2H]²⁺: 1336.2.

### Preparation of Example 1022

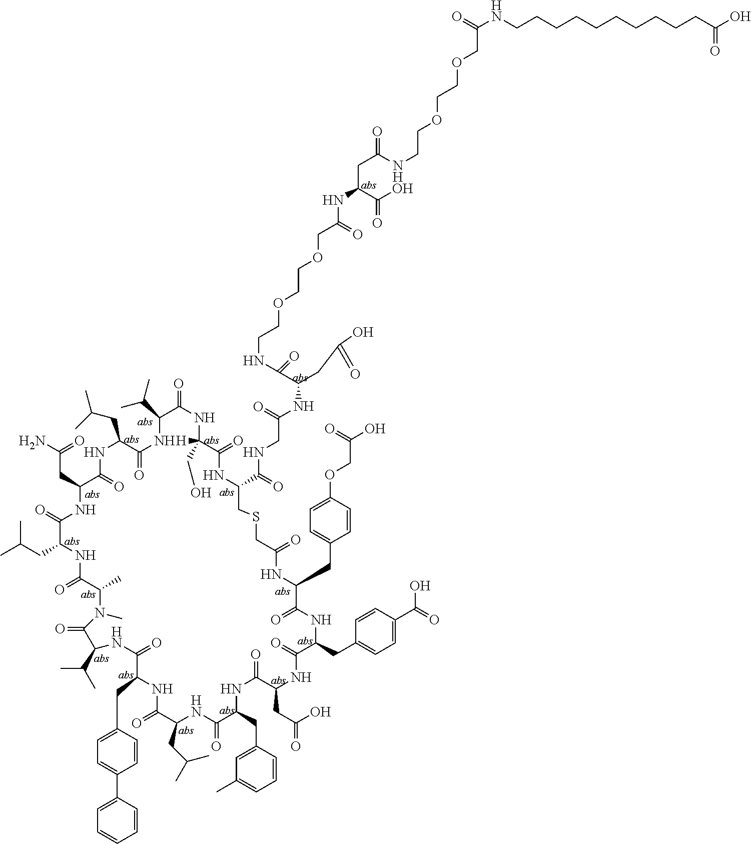 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)O)C(=O)NCCOCCOCC(=O)N[C@@H](CC(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1022 was prepared on a 50 μmol scale. The yield of the product was 30 mg, and its estimated purity by LCMS analysis was 82.4%. Analysis condition A: Retention time=1.28 min; ESI-MS(+) m/z [M+2H]²⁺: 1330.2.

### Preparation of Example 1023

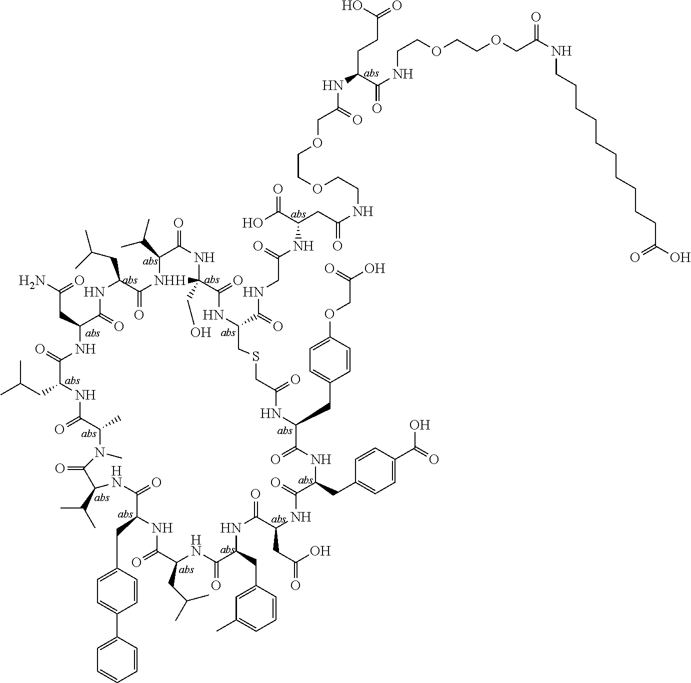 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)NCCOCCOCC(=O)N[C@@H](CCC(=O)O)C(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1023 was prepared on a 50 μmol scale. The yield of the product was 7.1 mg, and its estimated purity by LCMS analysis was 90.3%. Analysis condition B: Retention time=1.87 min; ESI-MS(+) m/z [M+3H]³⁺: 891.5.

### Preparation of Example 1024

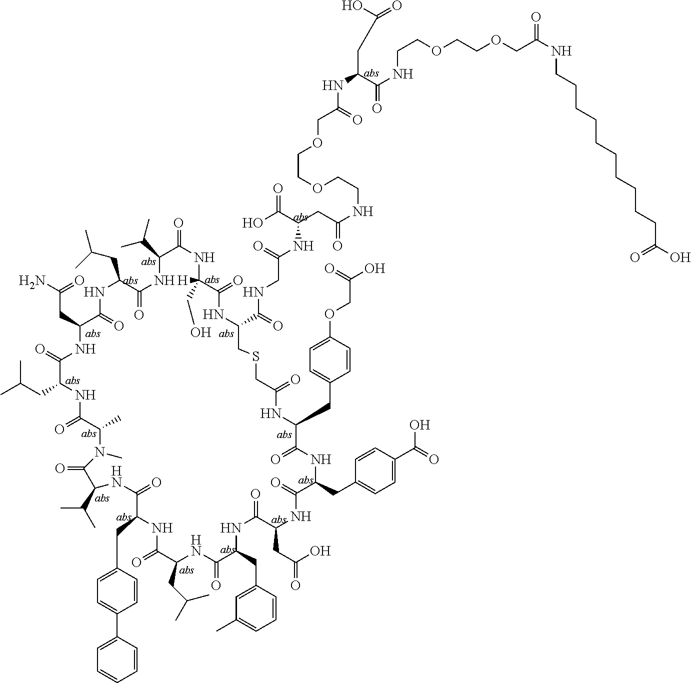 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)NCCOCCOCC(=O)N[C@@H](CC(=O)O)C(=O)NCCOCCOCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1024 was prepared on a 50 μmol scale. The yield of the product was 18.1 mg, and its estimated purity by LCMS analysis was 100%. Analysis condition A: Retention time=1.3 min; ESI-MS(+) m/z [M+2H]²⁺: 1329.2.

### Preparation of Example 1025

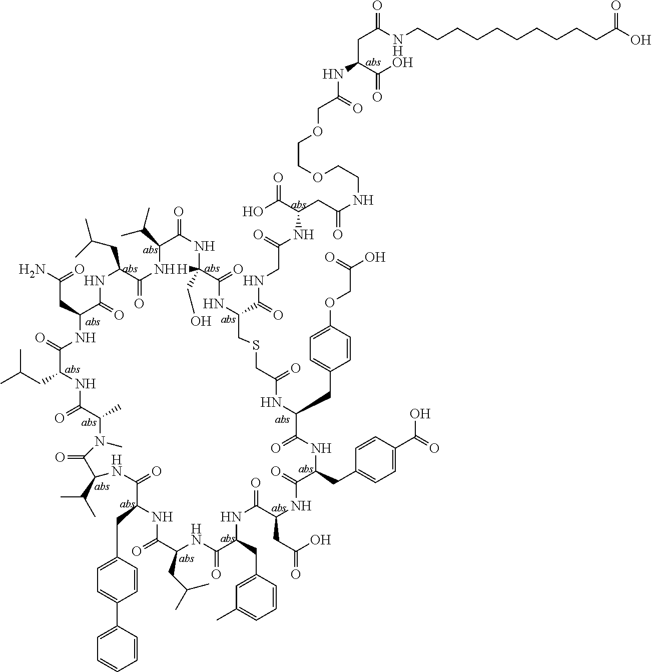 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CC(=O)NCCOCCOCC(=O)N[C@@H](CC(=O)NCCCCCCCCCCC(=O)O)C(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1025 was prepared on a 50 μmol scale. The yield of the product was 18.7 mg, and its estimated purity by LCMS analysis was 95%. Analysis condition A: Retention time=1.45 min; ESI-MS(+) m/z [M+2H]²⁺: 1257.1.

### Preparation of Example 1026

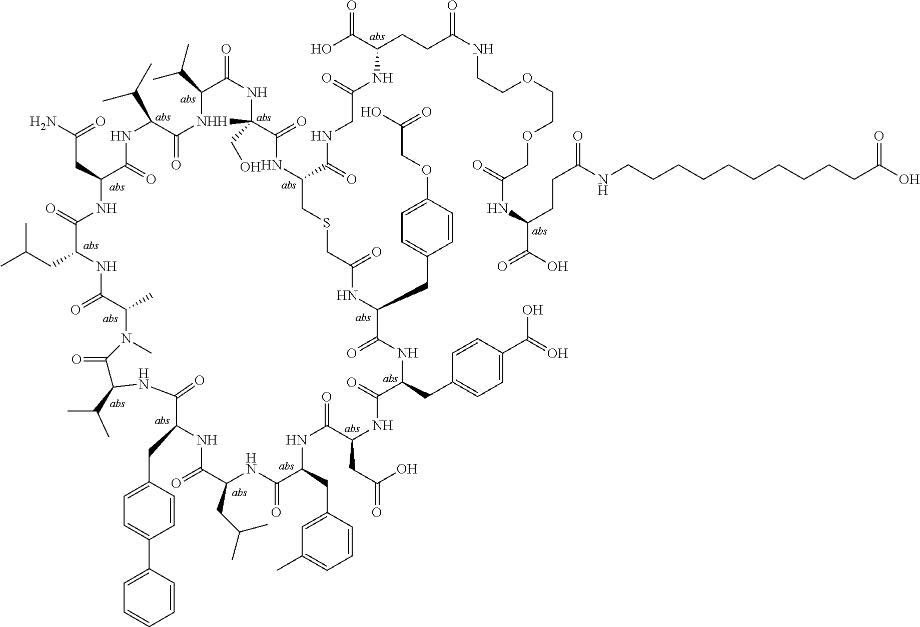 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCC(=O)N[C@@H](CCC(=O)NCCOCCOCC(=O)N[C@@H](CCC(=O)NCCCCCCCCCCC(=O)O)C(=O)O)C(=O)O)NC(=O)[C@H](CO)NC(=O)[C@H](C(C)C)NC(=O)[C@H](C(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1026 was prepared on a 50 μmol scale. The yield of the product was 13.2 mg, and its estimated purity by LCMS analysis was 90.6%. Analysis condition B: Retention time=2.04 min; ESI-MS(+) m/z [M+2H]²⁺: 1271.1.

### Preparation of Example 1027

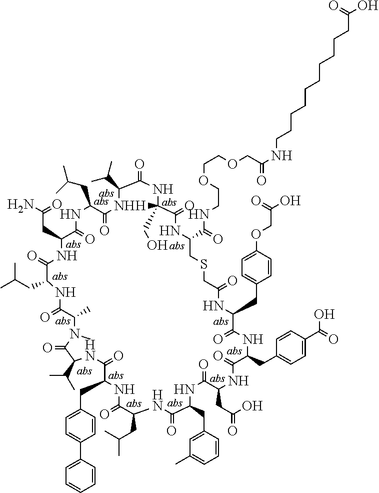 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1027 was prepared on a 50 μmol scale. The yield of the product was 18.8 mg, and its estimated purity by LCMS analysis was 93.1%. Analysis condition A: Retention time=1.44 min; ESI-MS(+) m/z [M+2H]²⁺: 1113.

### Preparation of Example 1028

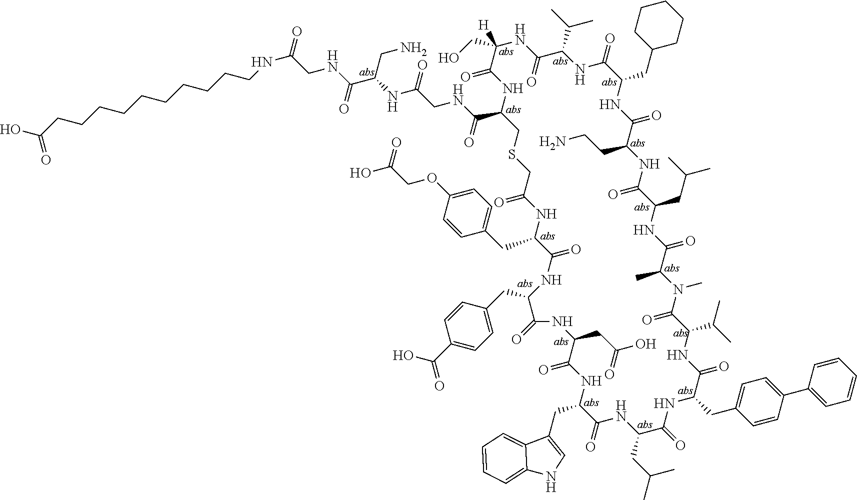

Example 1028 was prepared on a 50 μmol scale. The yield of the product was 36.7 mg, and its estimated purity by LCMS analysis was 91.4%. Analysis condition B: Retention time=1.73 min; ESI-MS(+) m/z [M+3H]²⁺: 778.

### Preparation of Example 1029

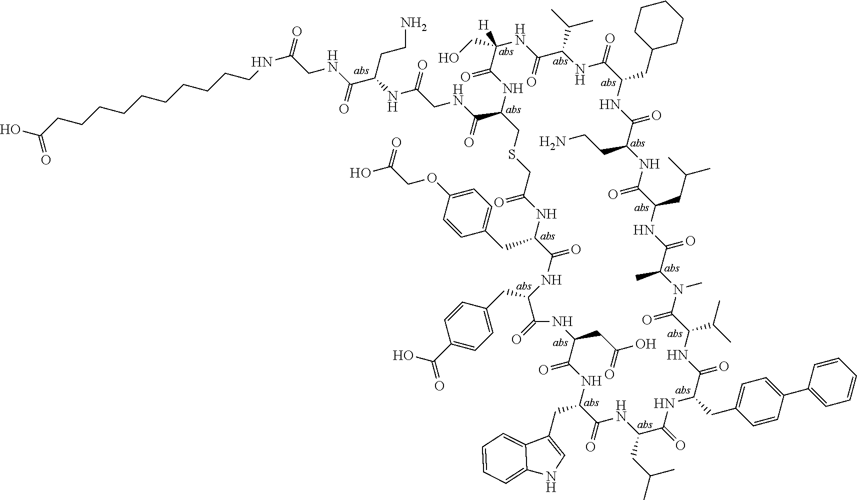

Example 1029 was prepared on a 50 μmol scale. The yield of the product was 29.7 mg, and its estimated purity by LCMS analysis was 88.50%. Analysis condition B: Retention time=2.54 min; ESI-MS(+) m/z [M+2H]²⁺: 1174.2.

### Preparation of Example 1030

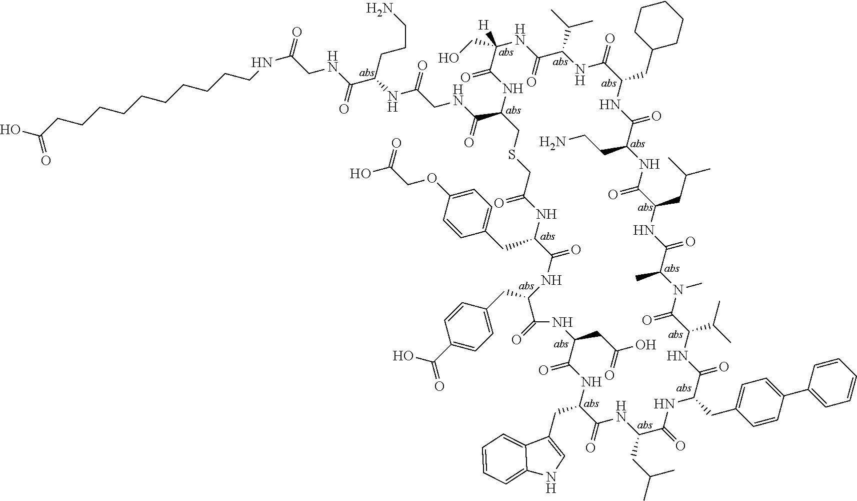

Example 1030 was prepared on a 50 μmol scale. The yield of the product was 24.8 mg, and its estimated purity by LCMS analysis was 91.2%. Analysis condition B: Retention time=1.84 min; ESI-MS(+) m/z [M+2H]²⁺: 1180.9.

### Preparation of Example 1031

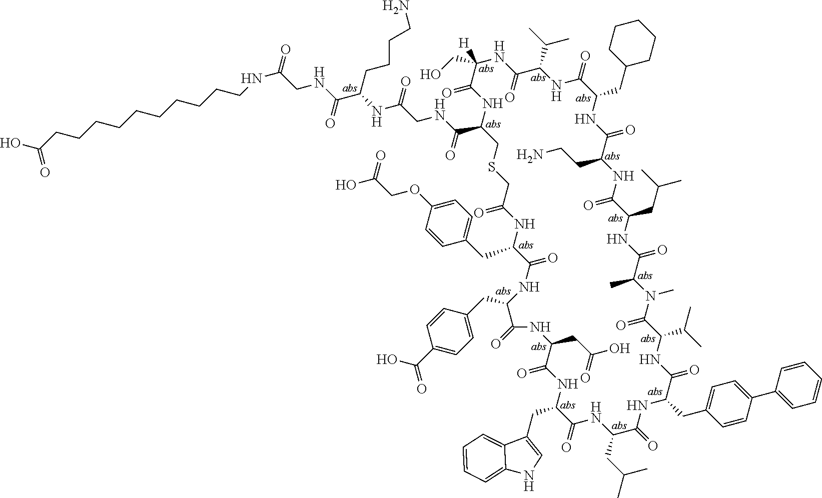

Example 1031 was prepared on a 50 μmol scale. The yield of the product was 21 mg, and its estimated purity by LCMS analysis was 92.3%. Analysis condition B: Retention time=1.73 min; ESI-MS(+) m/z [M+3H]²⁺: 792.

### Preparation of Example 1032

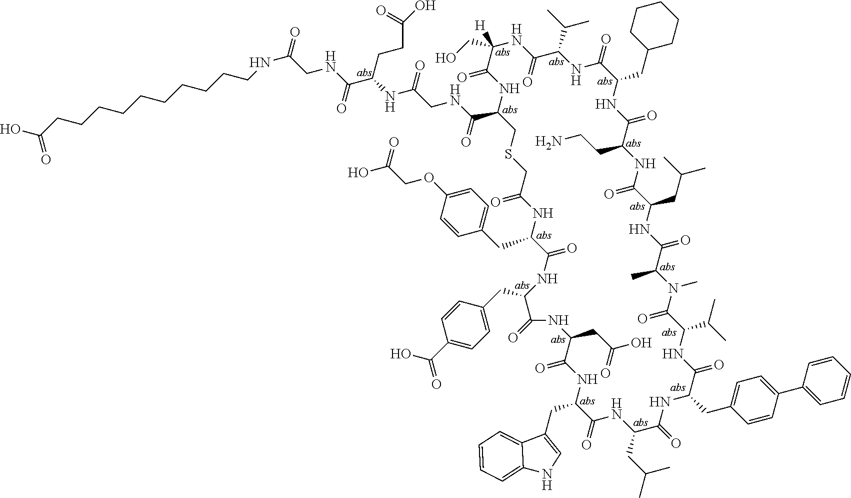

Example 1032 was prepared on a 50 μmol scale. The yield of the product was 30 mg, and its estimated purity by LCMS analysis was 85.9%. Analysis condition A: Retention time=1.5 min; ESI-MS (+) m/z [M+2H]²⁺: 1188.

### Preparation of Example 1033

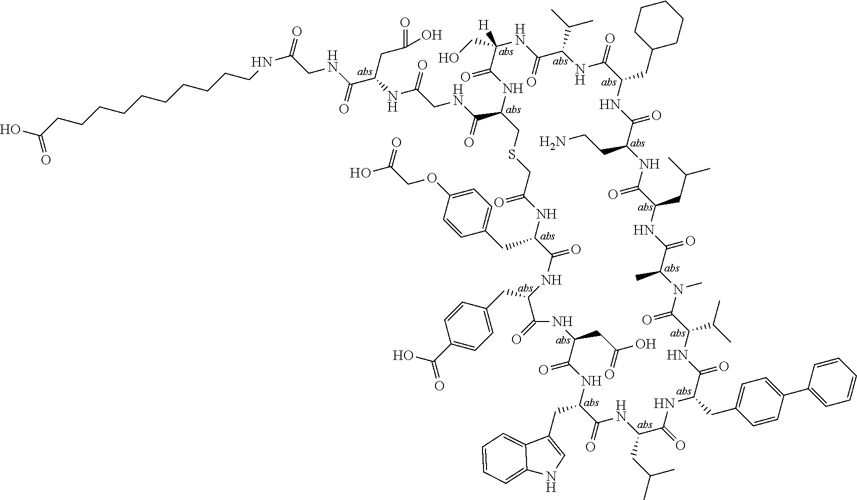

Example 1033 was prepared on a 50 μmol scale. The yield of the product was 29.3 mg, and its estimated purity by LCMS analysis was 935%. Analysis condition B: Retention time=2.63 min; ESI-MS(+) m/z [M+2H]²⁺: 1180.9.

### Preparation of Example 1034

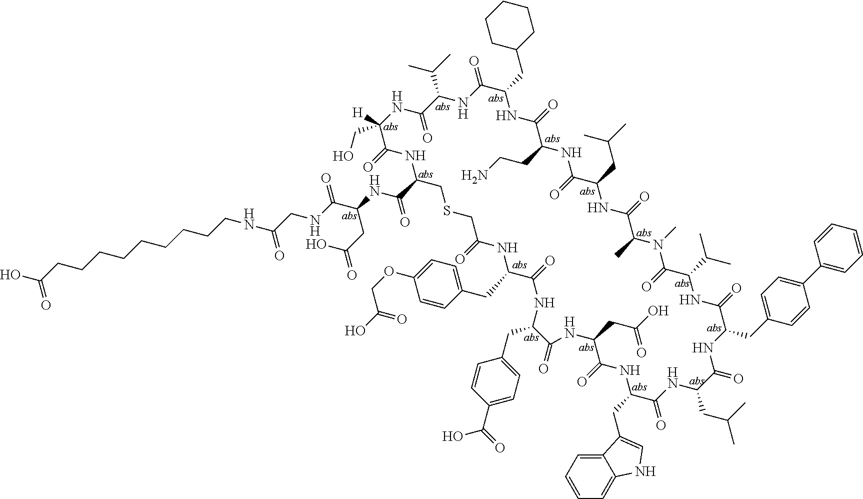

Example 1034 was prepared on a 50 μmol scale. The yield of the product was 33.2 mg, and its estimated purity by LCMS analysis was 97.8%. Analysis condition B: Retention time=1.81 min; ESI-MS(+) m/z [M+2H]²⁺: 1153.

### Preparation of Example 1035

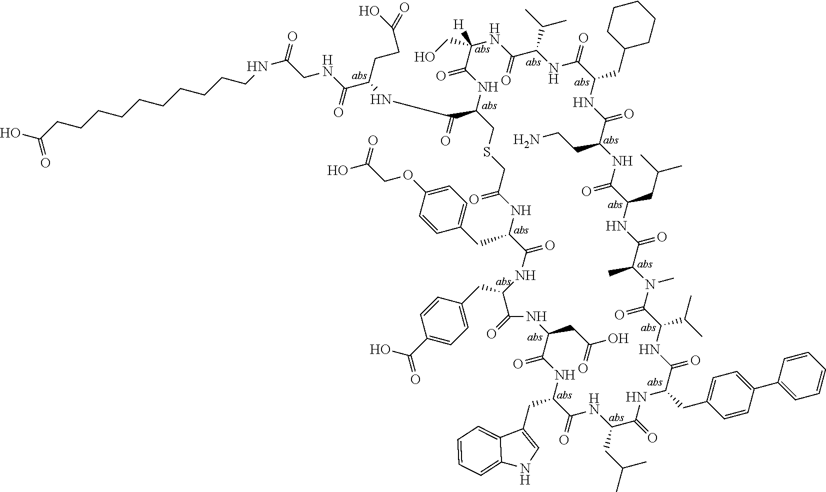

Example 1035 was prepared on a 50 μmol scale. The yield of the product was 15.8 mg, and its estimated purity by LCMS analysis was 95.3%. Analysis condition B: Retention time=1.93 min; ESI-MS(+) m/z [M+2H]²⁺: 1160.1.

### Preparation of Example 1036

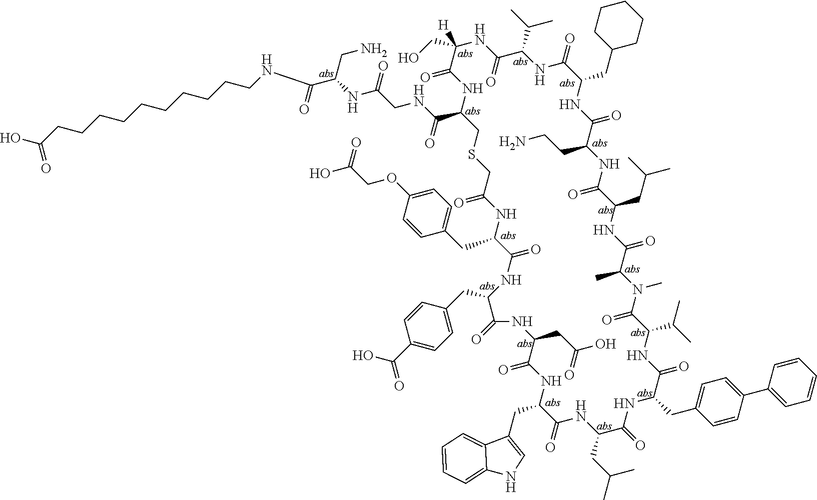

Example 1036 was prepared on a 50 μmol scale. The yield of the product was 22.8 mg, and its estimated purity by LCMS analysis was 86.500. Analysis condition B: Retention time=1.87 min; ESI-MS(+) m/z [M+2H]²⁺: 1137.9.

### Preparation of Example 1037

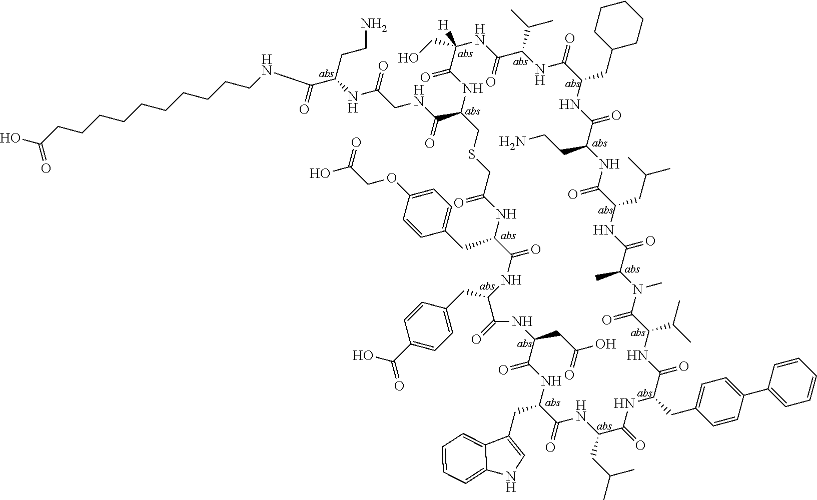

Example 1037 was prepared on a 50 μmol scale. The yield of the product was 13.9 mg, and its estimated purity by LCMS analysis was 94.2%. Analysis condition B: Retention time=1.86 min; ESI-MS(+) m/z [M+2H]²⁺: 1144.9.

### Preparation of Example 1038

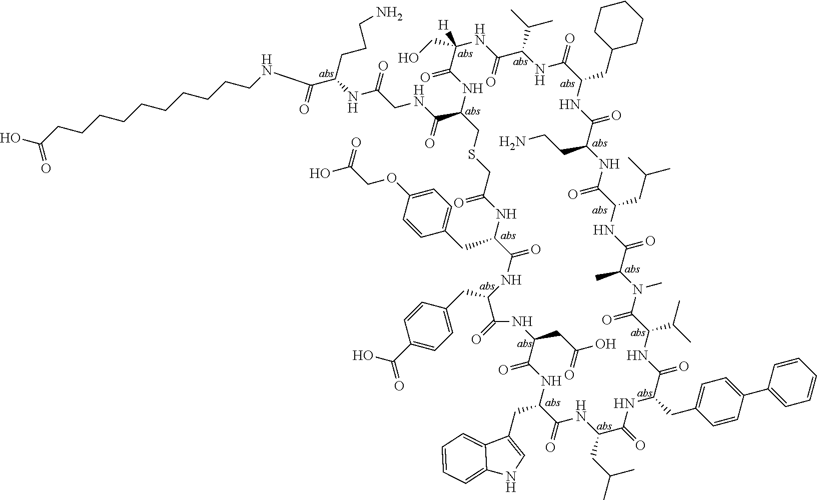

Example 1038 was prepared on a 50 μmol scale. The yield of the product was 15.8 mg, and its estimated purity by LCMS analysis was 93.9%. Analysis condition A: Retention time=1.63 min; ESI-MS(+) m/z [M+2H]²⁺: 1152.1.

### Preparation of Example 1039

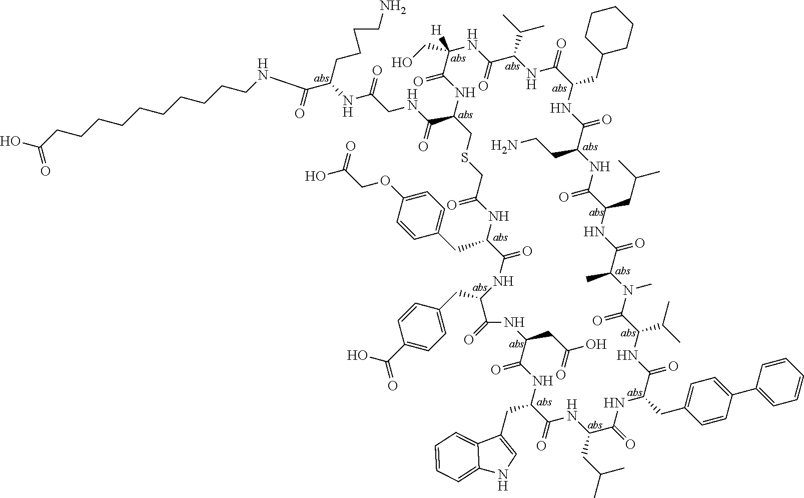

Example 1039 was prepared on a 50 μmol scale. The yield of the product was 17.4 mg, and its estimated purity by LCMS analysis was 93.5%. Analysis condition B: Retention time=1.82 min; ESI-MS(+) m/z [M+2H]²⁺: 1159.

### Preparation of Example 1040

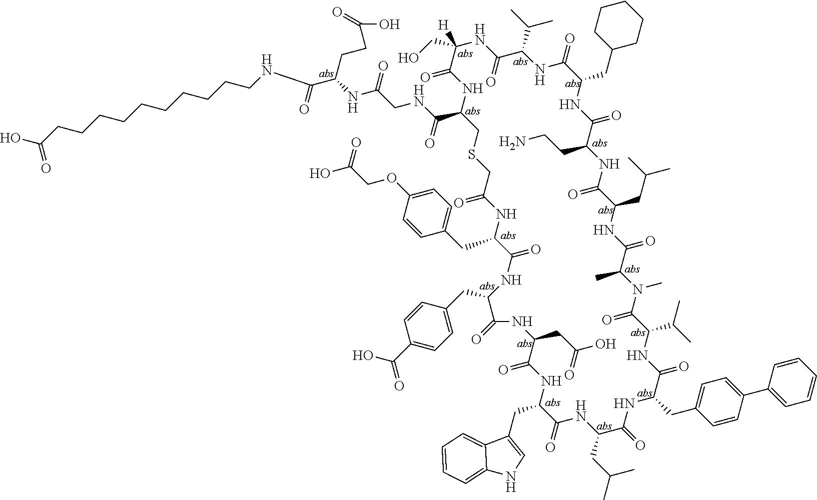

Example 1040 was prepared on a 50 μmol scale. The yield of the product was 20.6 mg, and its estimated purity by LCMS analysis was 98.40%. Analysis condition B: Retention time=1.96 min; ESI-MS(+) m/z [M+2H]²⁺: 1159.1.

### Preparation of Example 1041

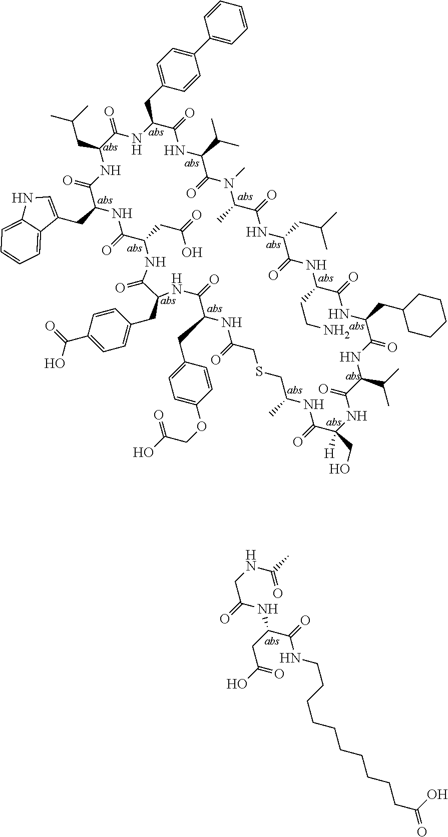

Example 1041 was prepared on a 50 μmol scale. The yield of the product was 22 mg, and its estimated purity by LCMS analysis was 99.2%. Analysis condition B: Retention time=1.92 min; ESI-MS(+) m/z [M+2H]²⁺: 1153.1.

### Preparation of Example 1042

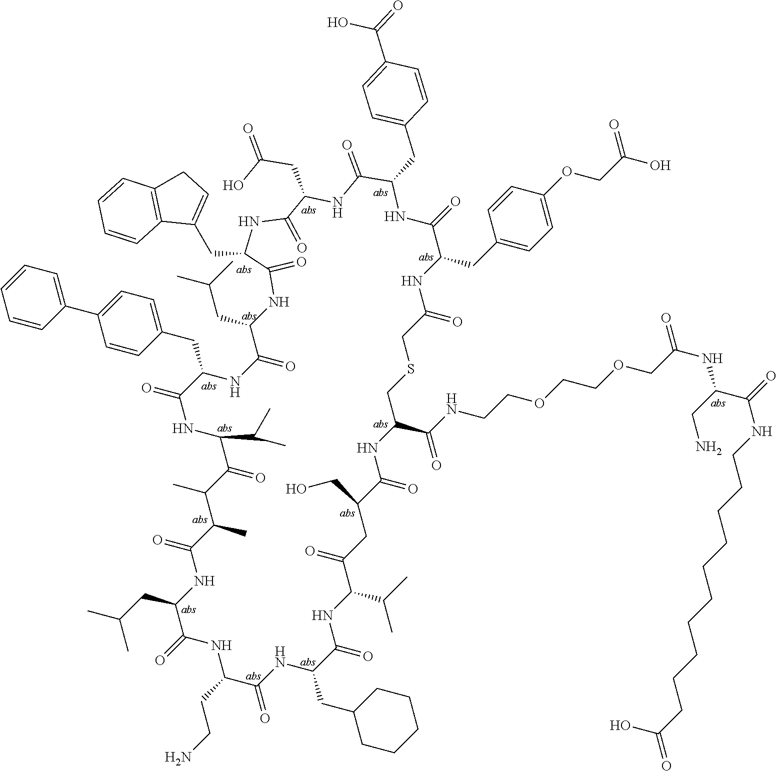

Example 1042 was prepared on a 50 μmol scale. The yield of the product was 29.1 mg, and its estimated purity by LCMS analysis was 100%. Analysis condition B: Retention time=1.74 min; ESI-MS(+) m/z [M+2H]²⁺: 1182.

### Preparation of Example 1043

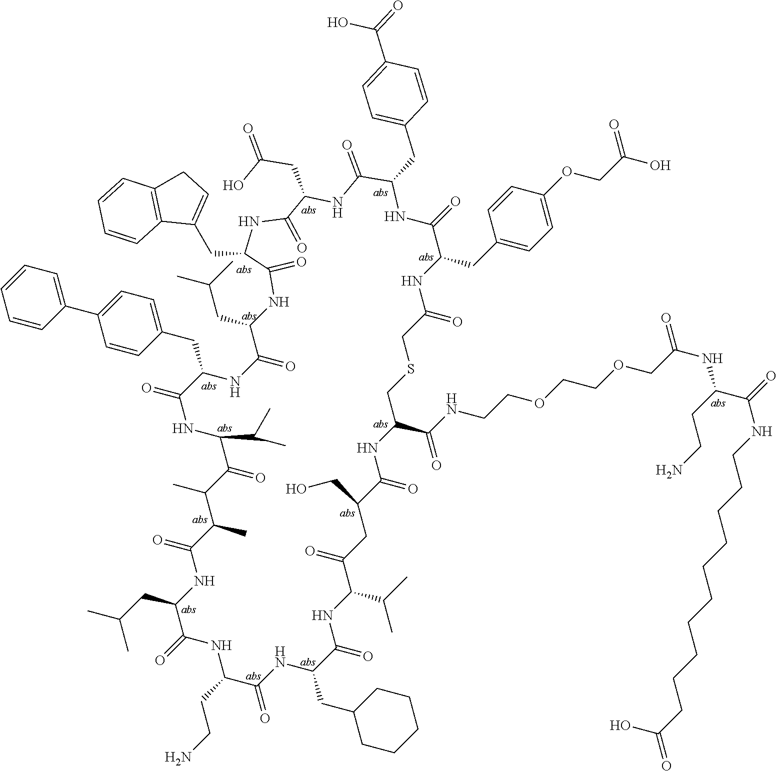

Example 1043 was prepared on a 50 μmol scale. The yield of the product was 20.4 mg, and its estimated purity by LCMS analysis was 87.7%. Analysis condition B: Retention time=1.89 min; ESI-MS(+) m/z [M+2H]²⁺: 1189.

### Preparation of Example 1044

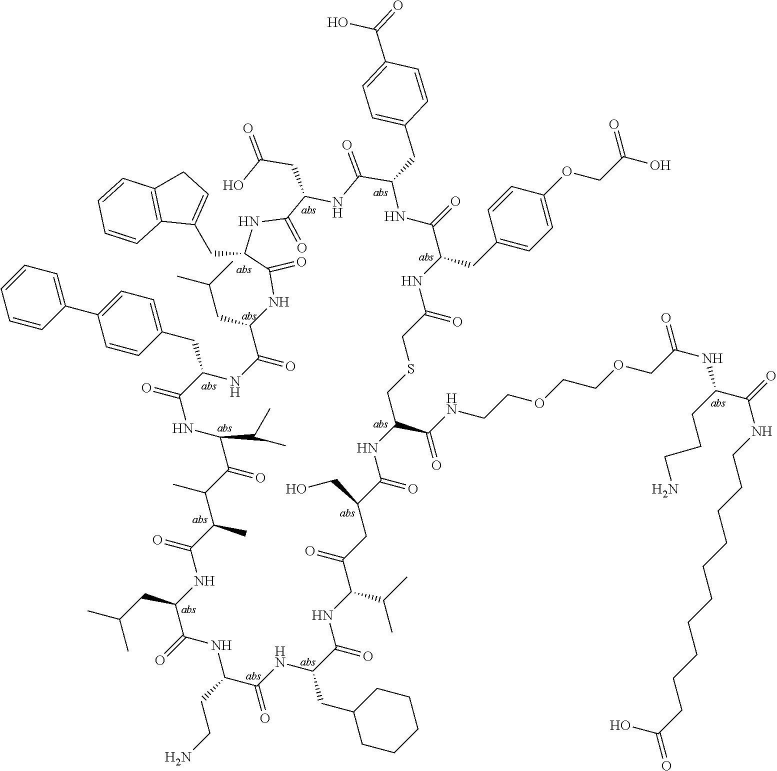

Example 1044 was prepared on a 50 μmol scale. The yield of the product was 23.5 mg, and its estimated purity by LCMS analysis was 96.7%. Analysis condition B: Retention time=1.85 min; ESI-MS(+) m/z [M+2H]²⁺: 1196.

### Preparation of Example 1045

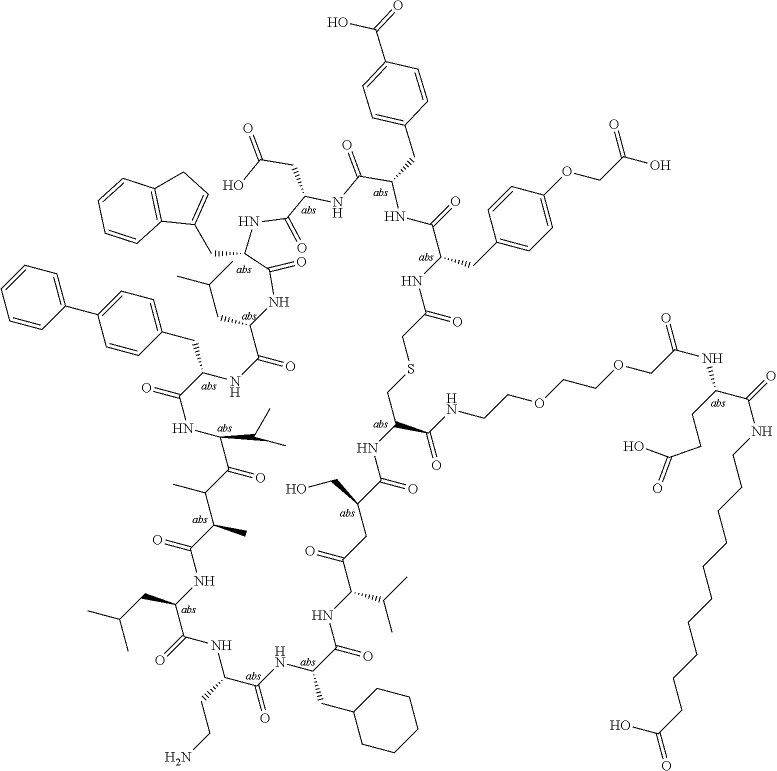

Example 1045 was prepared on a 50 μmol scale. The yield of the product was 24.9 mg, and its estimated purity by LCMS analysis was 99.1%. Analysis condition A: Retention time=1.59 min; ESI-MS(+) m/z [M+2H]²⁺: 1204.

### Preparation of Example 1046

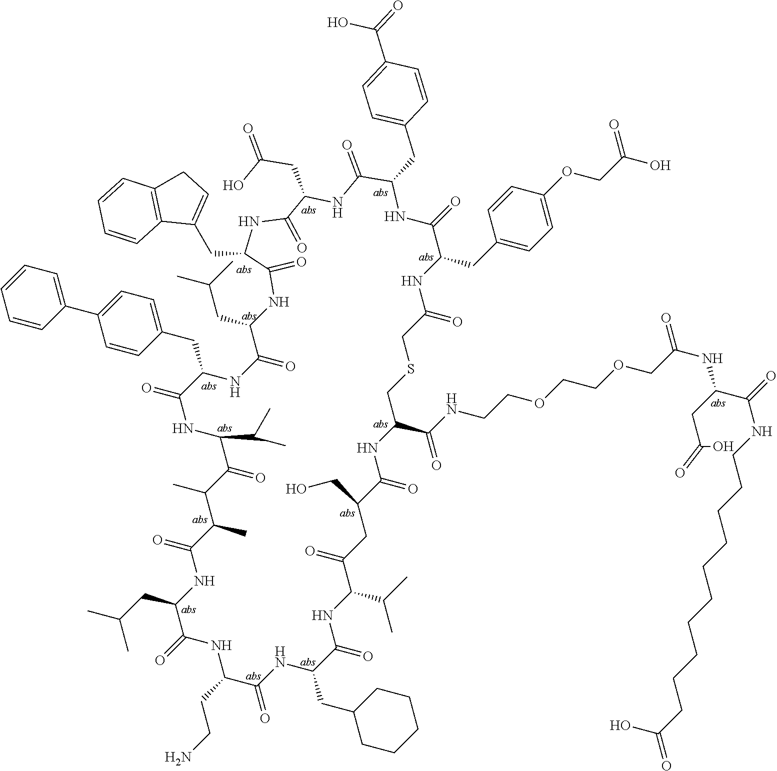

Example 1046 was prepared on a 50 μmol scale. The yield of the product was 41.2 mg, and its estimated purity by LCMS analysis was 94.9%. Analysis condition A: Retention time=1.58 min; ESI-MS(+) m/z [M+2H]²⁺: 1196.9.

### Preparation of Example 1047

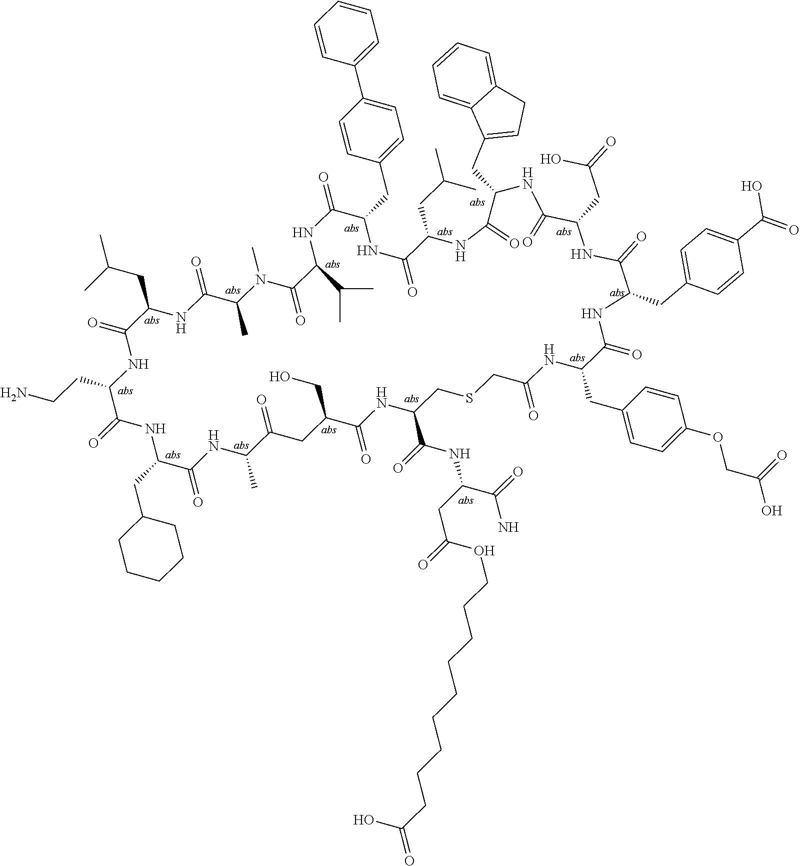 [SMILES: CC(C)C[C@@H]1NC(=O)[C@H](CC2=CCc3ccccc32)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(=O)OCCCCCCCCCC(=O)O)C(N)=O)NC(=O)[C@H](CO)CC(=O)[C@H](C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1047 was prepared on a 50 μmol scale. The yield of the product was 28.6 mg, and its estimated purity by LCMS analysis was 91.6%. Analysis condition A: Retention time=1.62 min; ESI-MS(+) m/z [M+2H]²⁺: 1124.

### Preparation of Example 1048

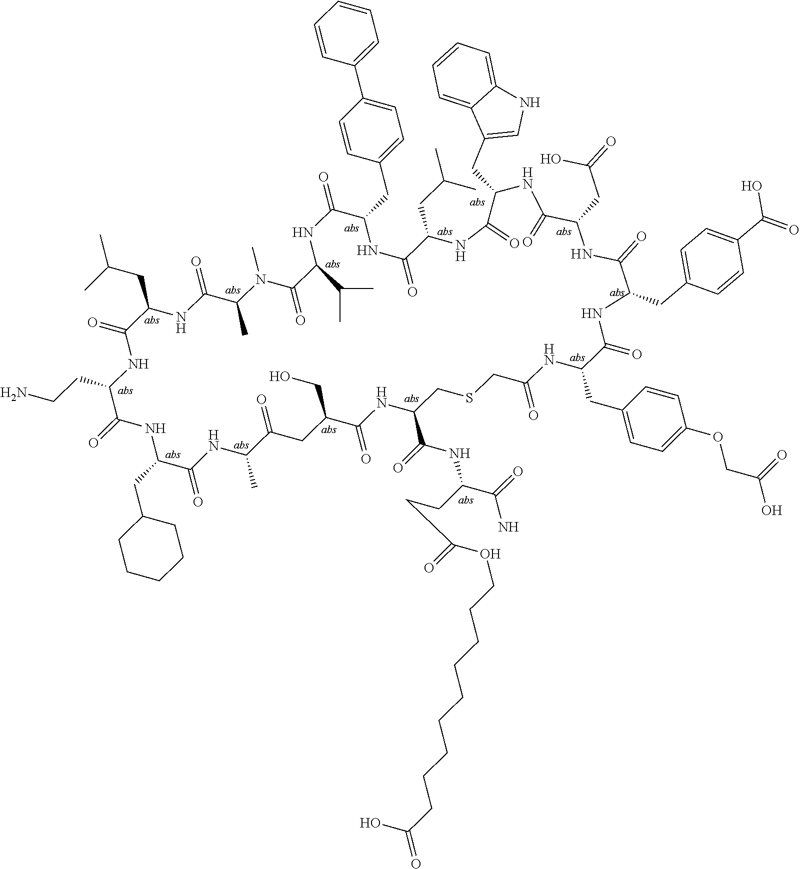 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCC(=O)OCCCCCCCCCC(=O)O)C(N)=O)NC(=O)[C@H](CO)CC(=O)[C@H](C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1048 was prepared on a 50 μmol scale. The yield of the product was 15 mg, and its estimated purity by LCMS analysis was 98.5%. Analysis condition B: Retention time=1.77 min; ESI-MS(+) m/z [M+2H]²⁺: 1131.

### Preparation of Example 1049

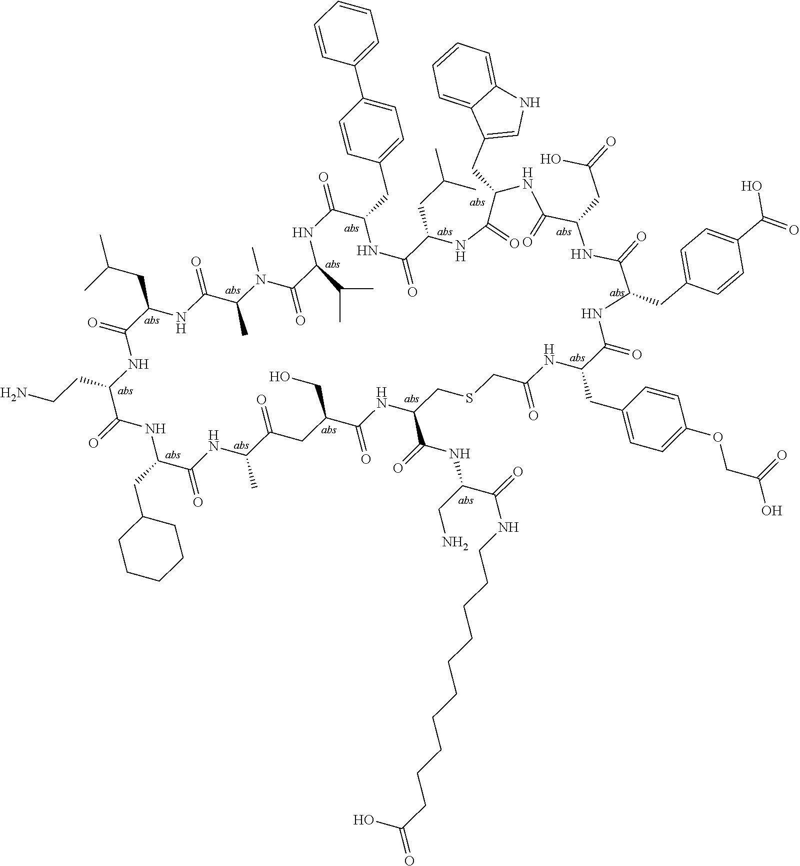 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCCCCCCCCCC(=O)O)NC(=O)[C@H](CO)CC(=O)[C@H](C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1049 was prepared on a 50 μmol scale. The yield of the product was 11 mg, and its estimated purity by LCMS analysis was 88.2%. Analysis condition A: Retention time=1.75 min; ESI-MS(+) m/z [M+2H]²⁺: 1110.2.

### Preparation of Example 1050

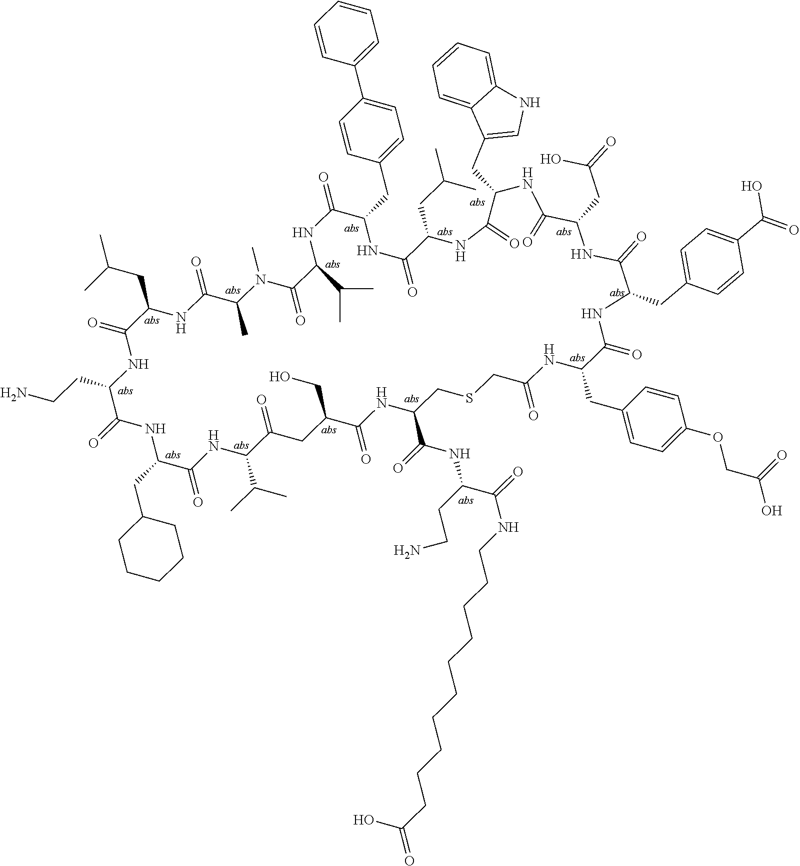 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1050 was prepared on a 50 μmol scale. The yield of the product was 10.1 mg, and its estimated purity by LCMS analysis was 94.6%. Analysis condition A: Retention time=1.62 min; ESI-MS(+) m/z [M+2H]²⁺: 1117.1

### Preparation of Example 1051

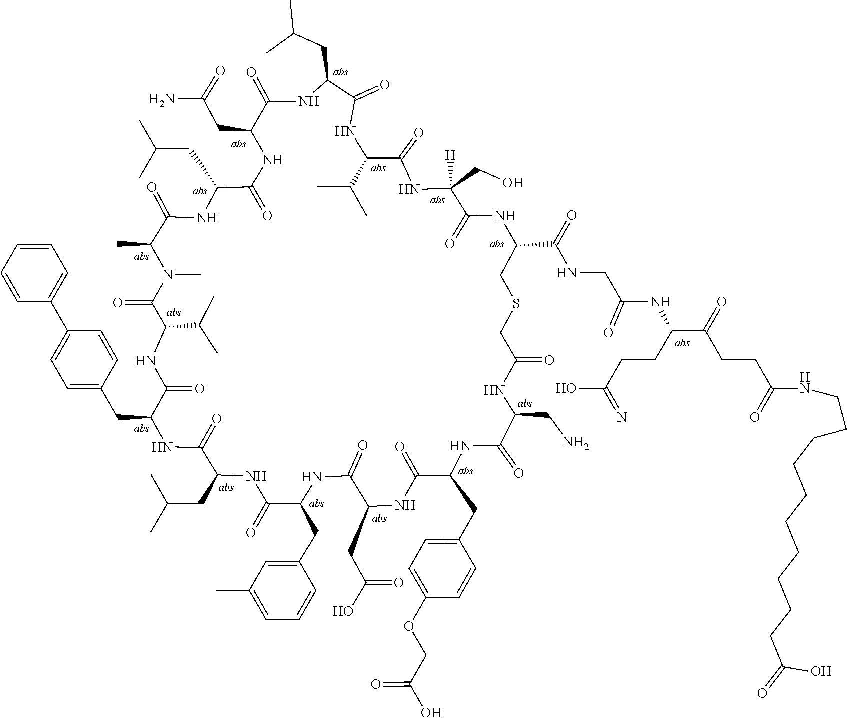 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CCC(=N)O)C(=O)CCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1051 was prepared on a 50 μmol scale. The yield of the product was 8.3 mg, and its estimated purity by LCMS analysis was 96.2%. Analysis condition B: Retention time=1.92, 1.97 min; ESI-MS(+) m/z [M+2H]²⁺: 1110.

### Preparation of Example 1052

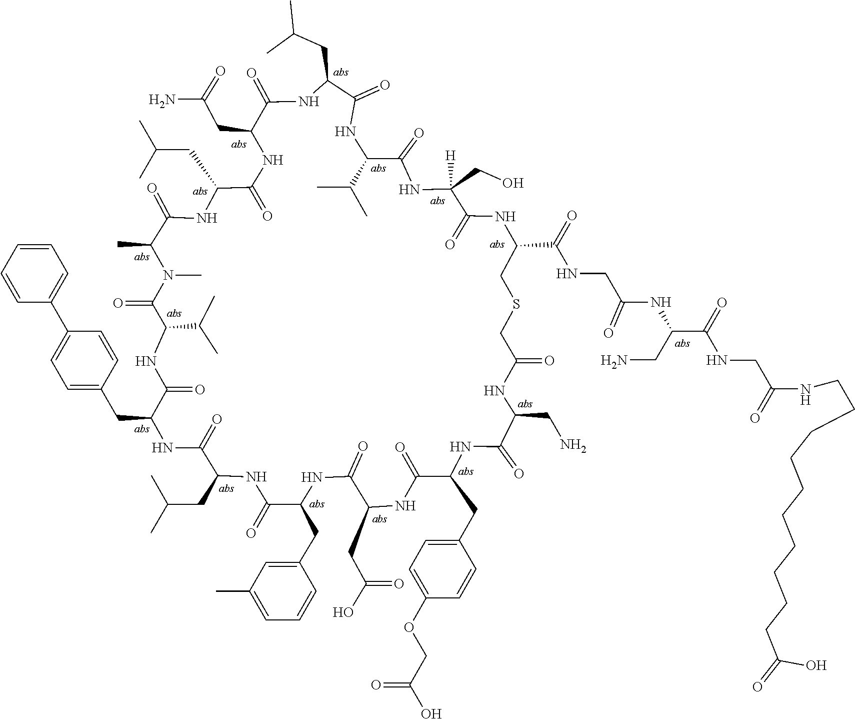 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CN)C(=O)NCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1052 was prepared on a 50 μmol scale. The yield of the product was 4.8 mg, and its estimated purity by LCMS analysis was 92.8%. Analysis condition A: Retention time=1.72 min; ESI-MS(+) m/z [M+2H]²⁺: 1088.2.

### Preparation of Example 1053

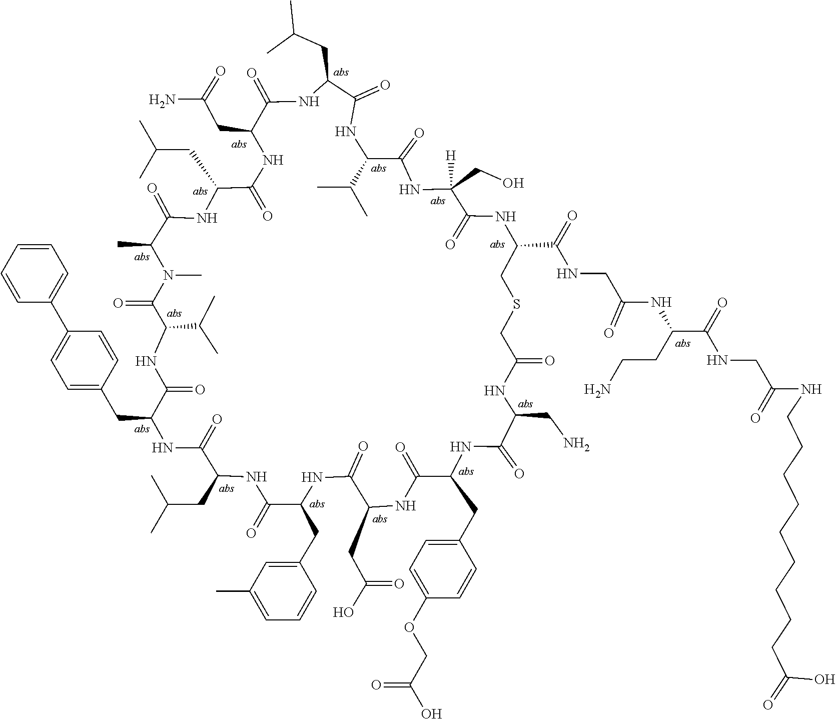 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CCN)C(=O)NCC(=O)NCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1053 was prepared on a 50 μmol scale. The yield of the product was 13.6 mg, and its estimated purity by LCMS analysis was 85.2%. Analysis condition B: Retention time=1.87 min; ESI-MS(+) m/z [M+3H]³⁺: 731.

### Preparation of Example 1054

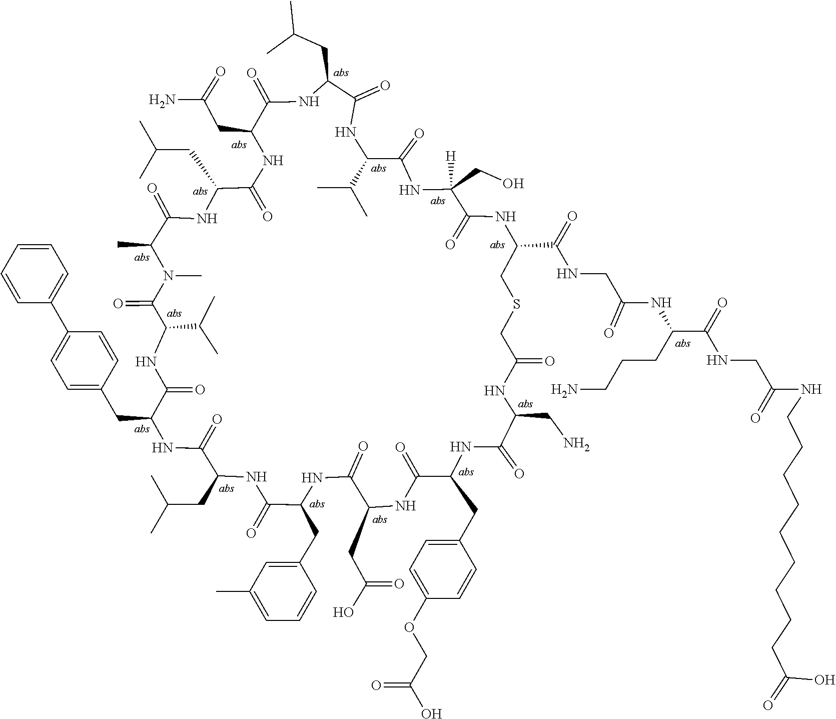 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CCCN)C(=O)NCC(=O)NCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1054 was prepared on a 50 μmol scale. The yield of the product was 1.4 mg, and its estimated purity by LCMS analysis was 80.2%. Analysis condition B: Retention time=1.99 min; ESI-MS(+) m/z [M+2H]²⁺: 1102.2.

### Preparation of Example 1055

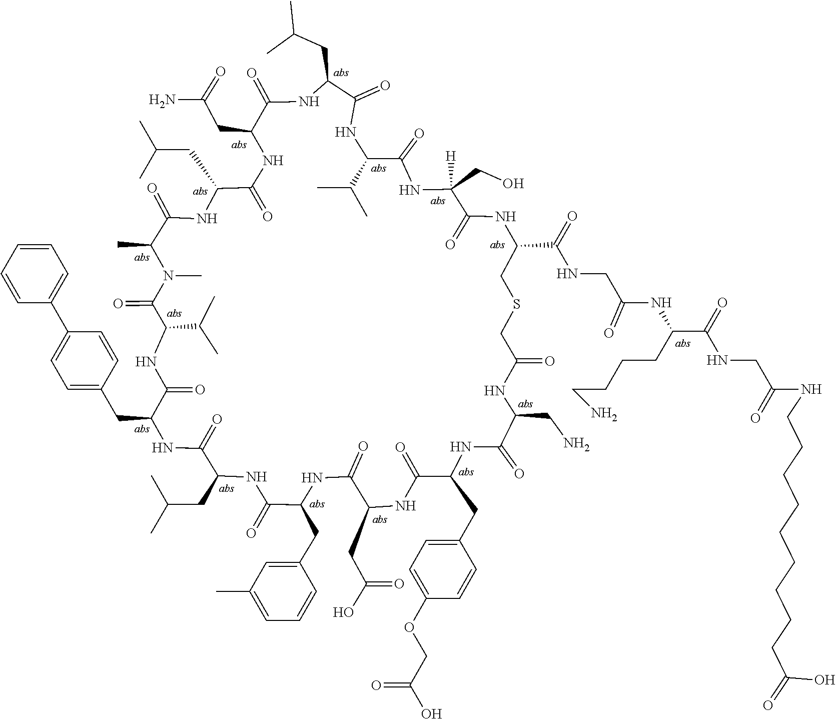 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CCCCN)C(=O)NCC(=O)NCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1055 was prepared on a 50 μmol scale. The yield of the product was 3.9 mg, and its estimated purity by LCMS analysis was 95.7%. Analysis condition B: Retention time=1.93 min; ESI-MS(+) m/z [M+2H]²⁺: 1110.

### Preparation of Example 1056

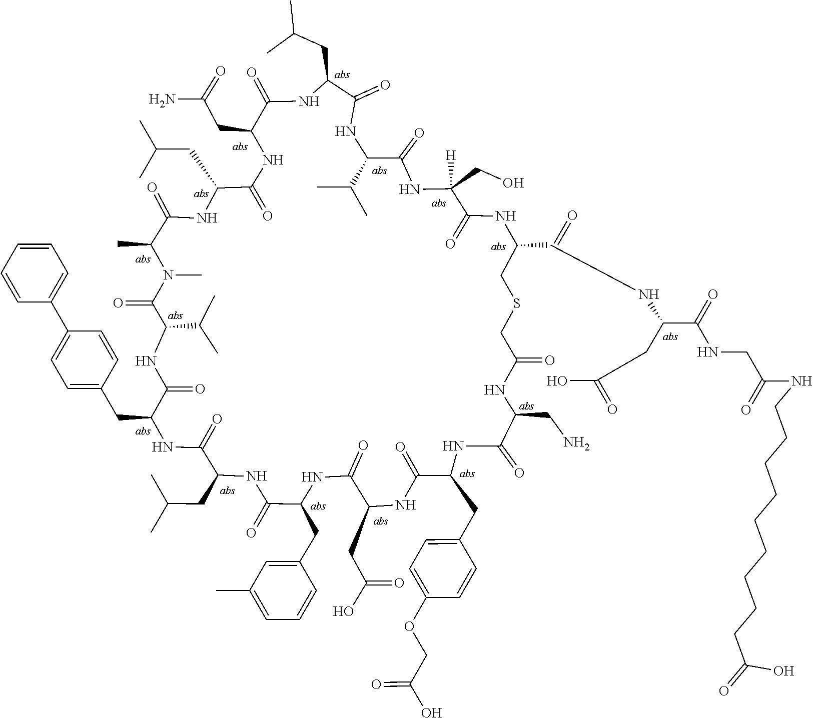

Example 1056 was prepared on a 50 μmol scale. The yield of the product was 2.2 mg, and its estimated purity by LCMS analysis was 85%. Analysis condition A: Retention time=1.65 min; ESI-MS(+) m/z [M+2H]²⁺: 1074.7.

### Preparation of Example 1057

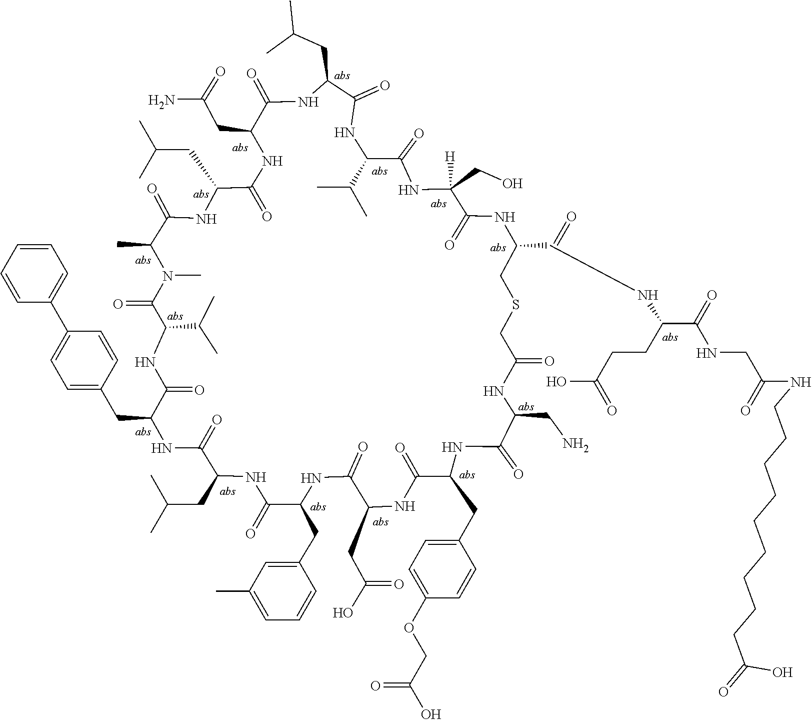

Example 1057 was prepared on a 50 μmol scale. The yield of the product was 1.9 mg, and its estimated purity by LCMS analysis was 84.9%. Analysis condition A: Retention time=1.61 min; ESI-MS(+) m/z [M+2H]²⁺: 1081.3.

### Preparation of Example 1058

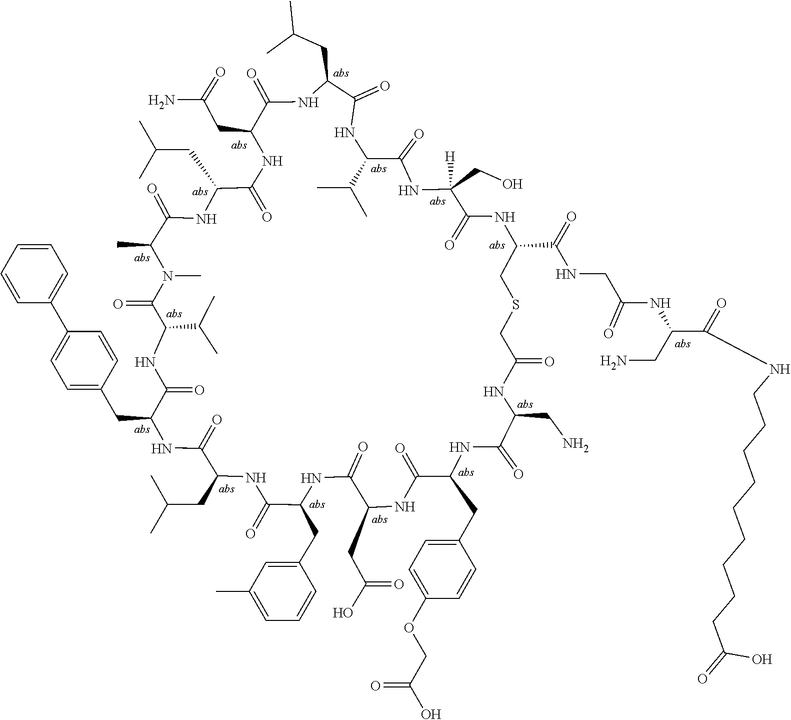

Example 1058 was prepared on a 50 μmol scale. The yield of the product was 7.4 mg, and its estimated purity by LCMS analysis was 100%. Analysis condition A: Retention time=1.73 min; ESI-MS(+) m/z [M+2H]²⁺: 1060.2.

### Preparation of Example 1059

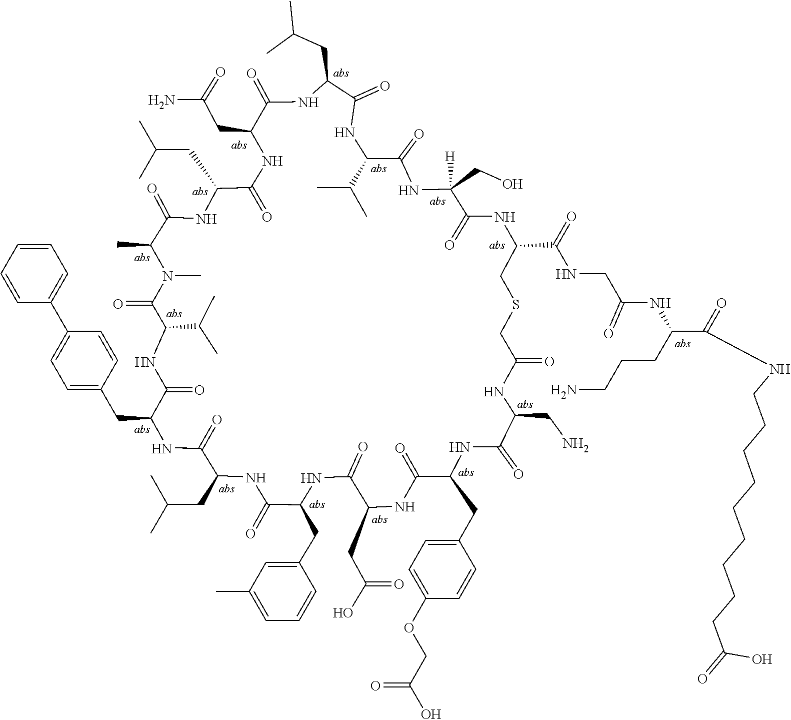

Example 1059 was prepared on a 50 μmol scale. The yield of the product was 2.4 mg, and its estimated purity by LCMS analysis was 92.5%. Analysis condition B: Retention time=1.94 min; ESI-MS(+) m/z [M+2H]²⁺: 1074.1.

### Preparation of Example 1060

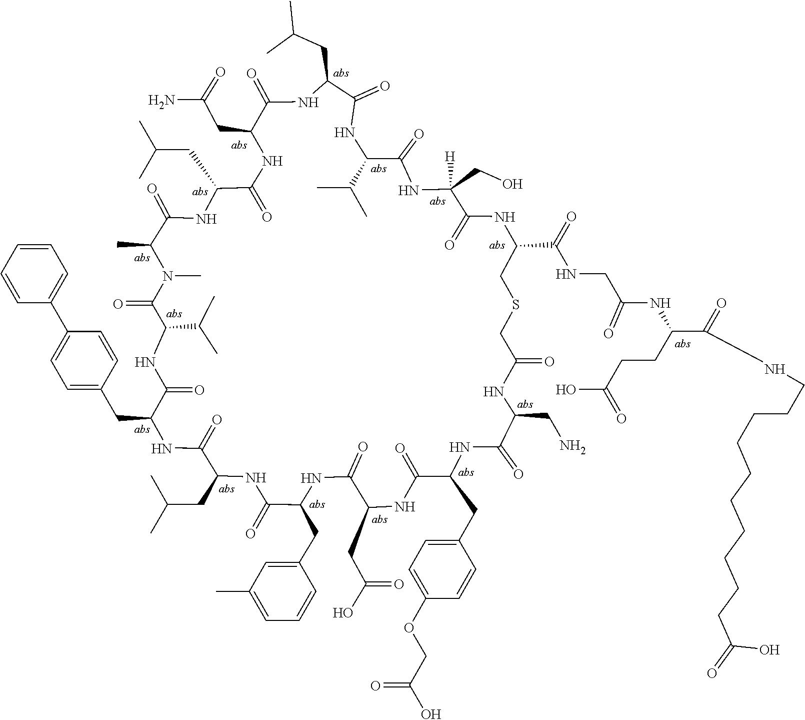

Example 1060 was prepared on a 50 μmol scale. The yield of the product was 7 mg, and its estimated purity by LCMS analysis was 88.3%. Analysis condition B: Retention time=1.87 min; ESI-MS(+) m/z [M+2H]²⁺: 1082.1.

### Preparation of Example 1061

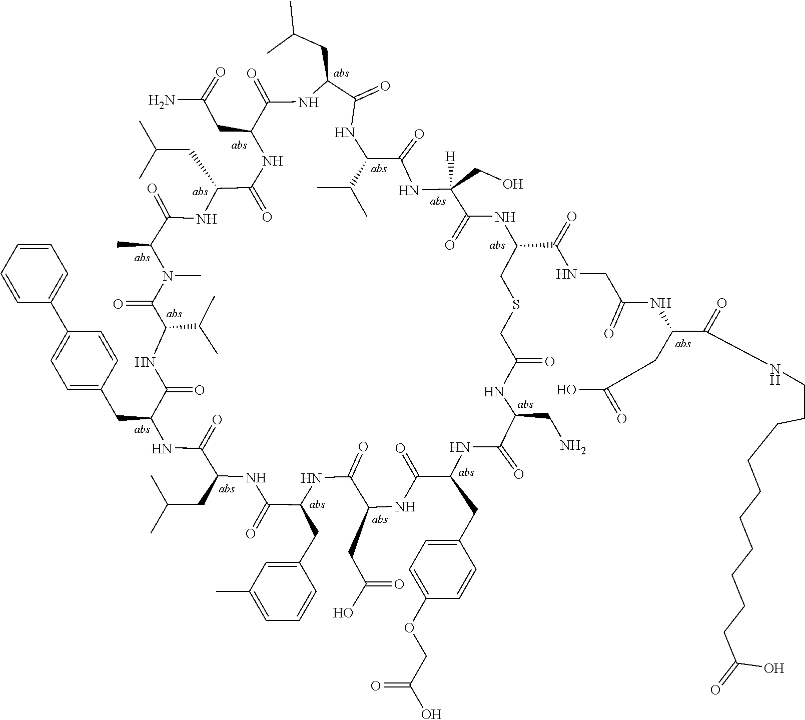

Example 1061 was prepared on a 50 μmol scale. The yield of the product was 17.5 mg, and its estimated purity by LCMS analysis was 99.1%. Analysis condition B: Retention time=1.95 min; ESI-MS(+) m/z [M+2H]²⁺: 1074.2.

### Preparation of Example 1062

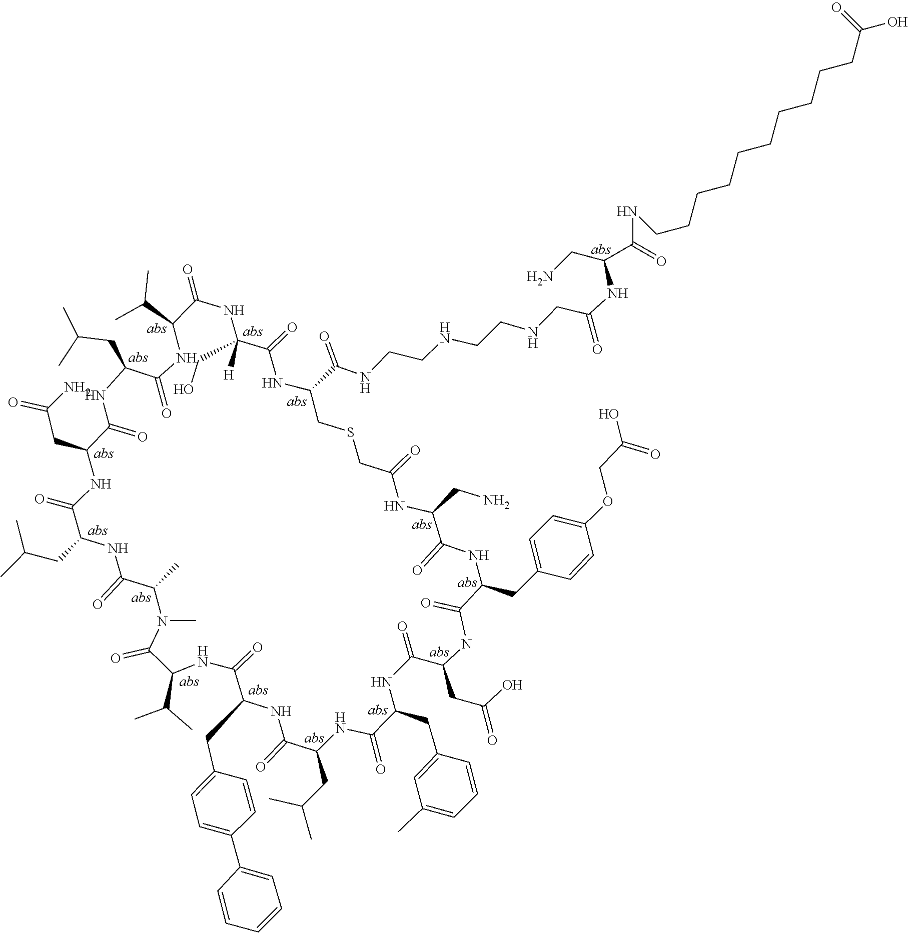

Example 1062 was prepared on a 50 μmol scale. The yield of the product was 4.5 mg, and its estimated purity by LCMS analysis was 93.9%. Analysis condition A: Retention time=1.72 min; ESI-MS(+) m/z [M+2H]²⁺: 1103.1.

### Preparation of Example 1063

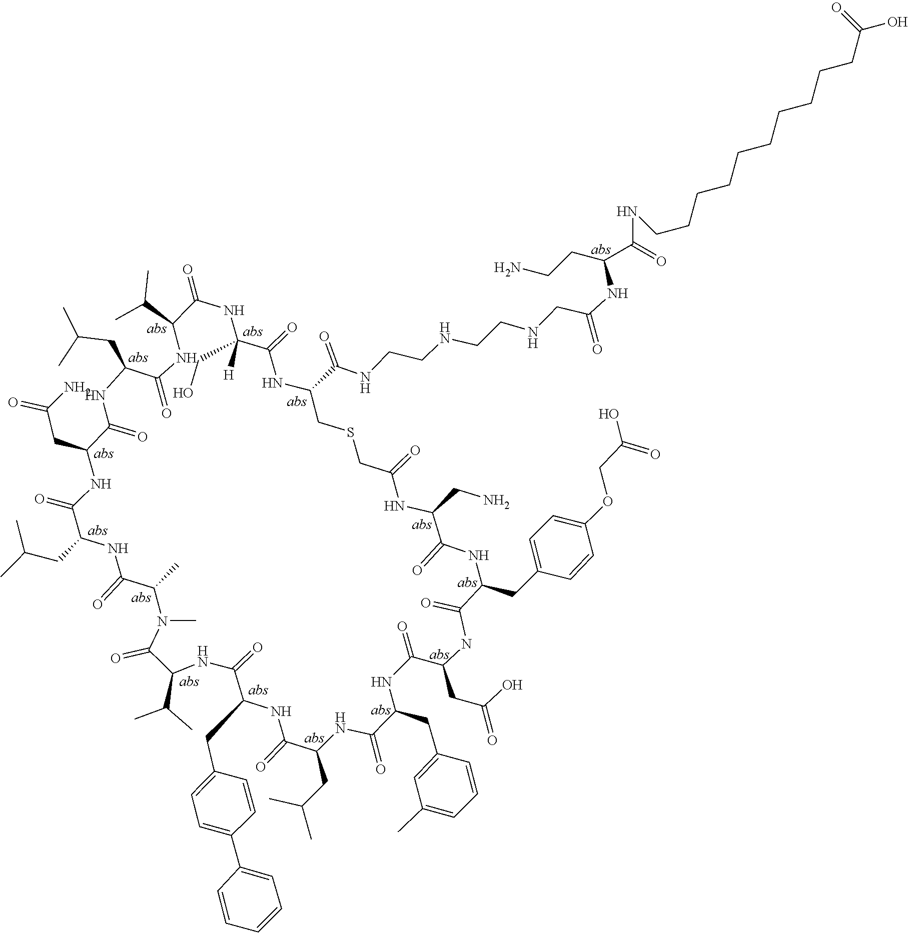

Example 1063 was prepared on a 50 μmol scale. The yield of the product was 7.2 mg, and its estimated purity by LCMS analysis was 67.9%. Analysis condition A: Retention time=1.64 min; ESI-MS(+) m/z [M+2H]²⁺: 1110.

### Preparation of Example 1064

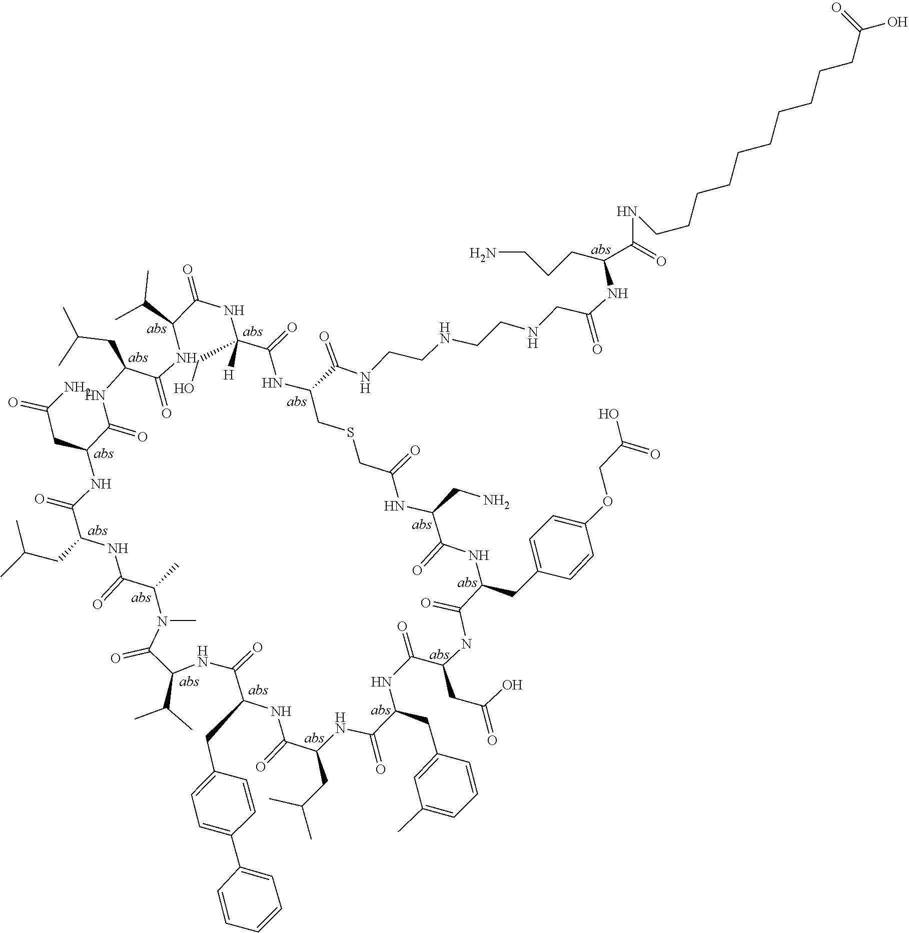

Example 1064 was prepared on a 50 μmol scale. The yield of the product was 4.7 mg, and its estimated purity by LCMS analysis was 100%. Analysis condition B: Retention time=1.93 min; ESI-MS(+) m/z [M+2H]²⁺: 1117.

### Preparation of Example 1065

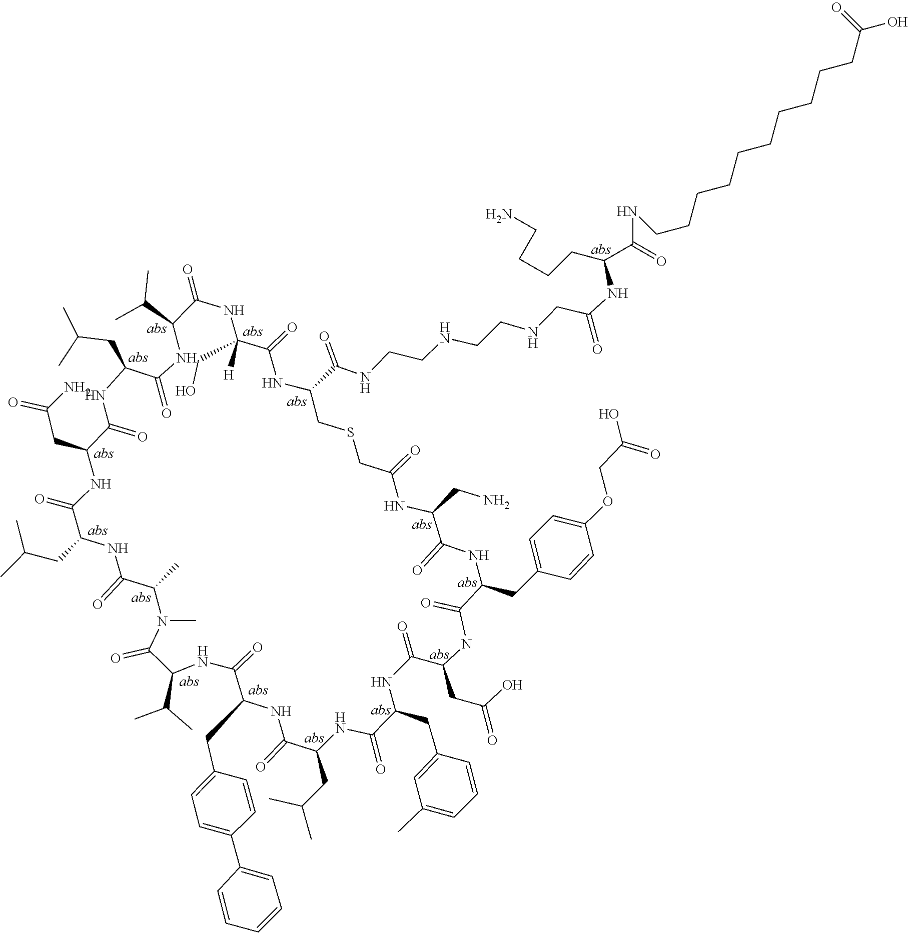

Example 1065 was prepared on a 50 μmol scale. The yield of the product was 10.6 mg, and its estimated purity by LCMS analysis was 95.7%. Analysis condition A: Retention time=1.78 min; ESI-MS(+) m/z [M+2H]²⁺: 1124.

### Preparation of Example 1066

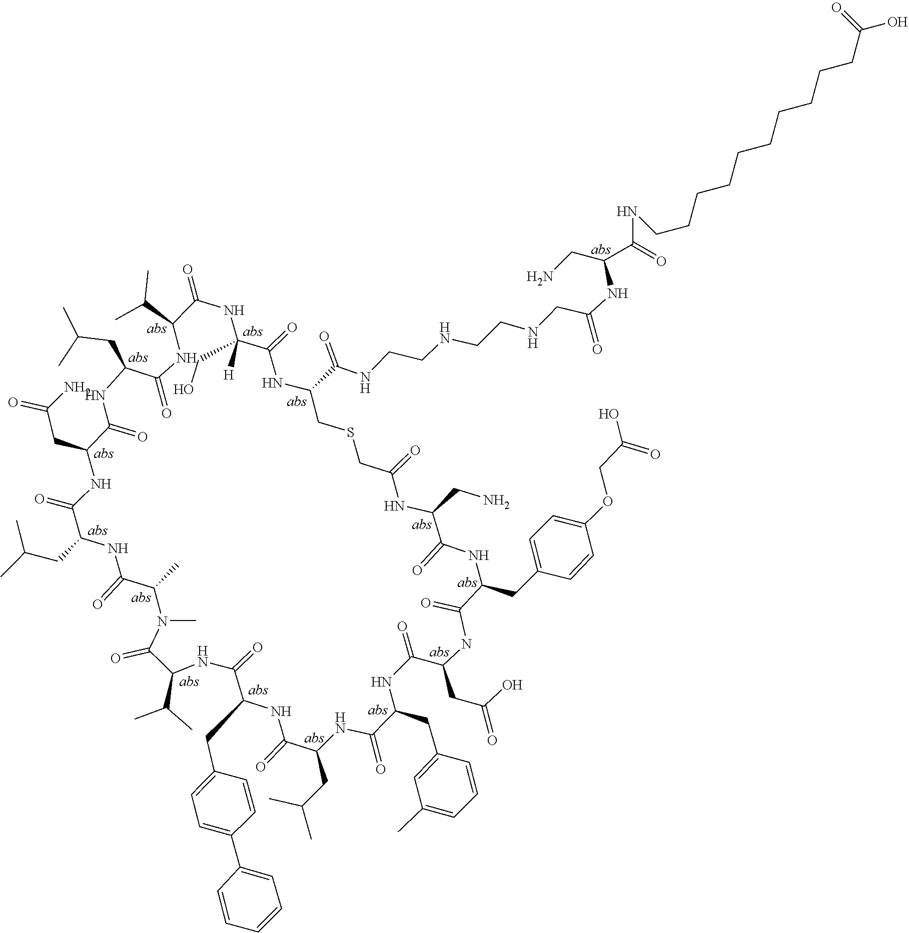

Example 1066 was prepared on a 50 μmol scale. The yield of the product was 6.1 mg, and its estimated purity by LCMS analysis was 100%. Analysis condition A: Retention time=1.56 min; ESI-MS(+) m/z [M+2H]²⁺: 1117.3.

### Preparation of Example 1067

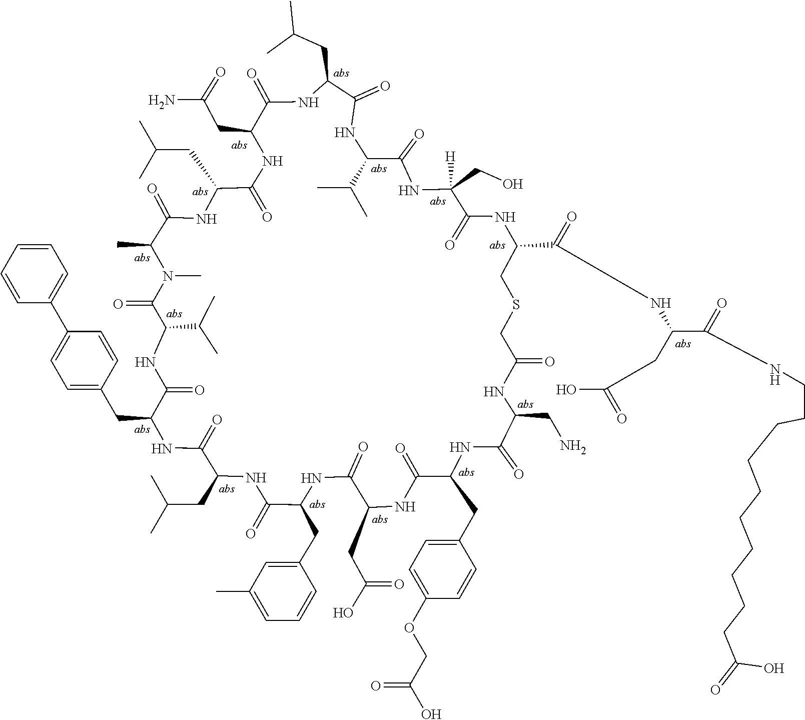

Example 1067 was prepared on a 50 μmol scale. The yield of the product was 10.6 mg, and its estimated purity by LCMS analysis was 99.3%. Analysis condition B: Retention time=1.99 min; ESI-MS(+) m/z [M+2H]²⁺: 1046.

### Preparation of Example 1068

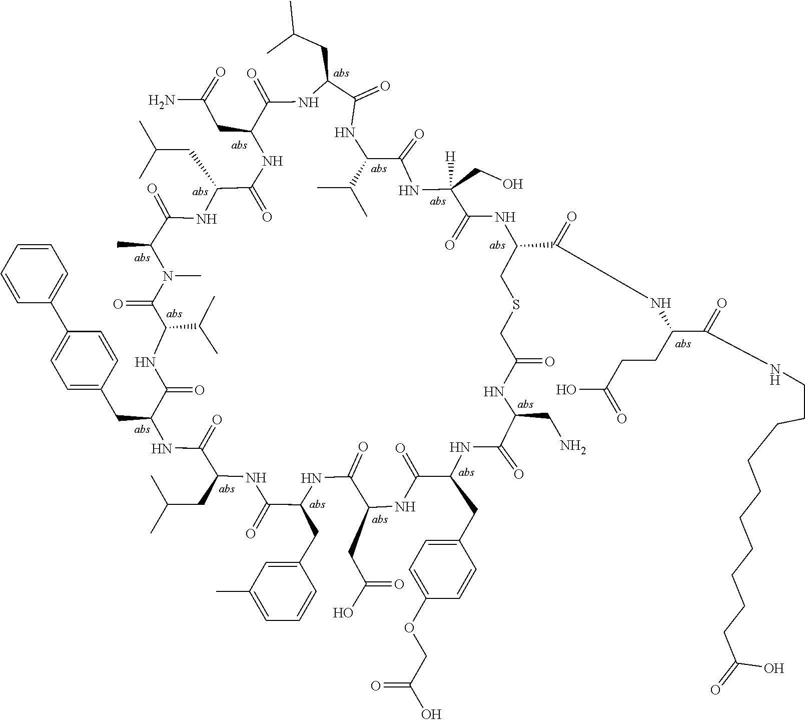

Example 1068 was prepared on a 50 μmol scale. The yield of the product was 8 mg, and its estimated purity by LCMS analysis was 88.7%. Analysis condition A: Retention time=1.48, 1.56 min; ESI-MS(+) m/z [M+2H]²⁺: 1053.

### Preparation of Example 1069

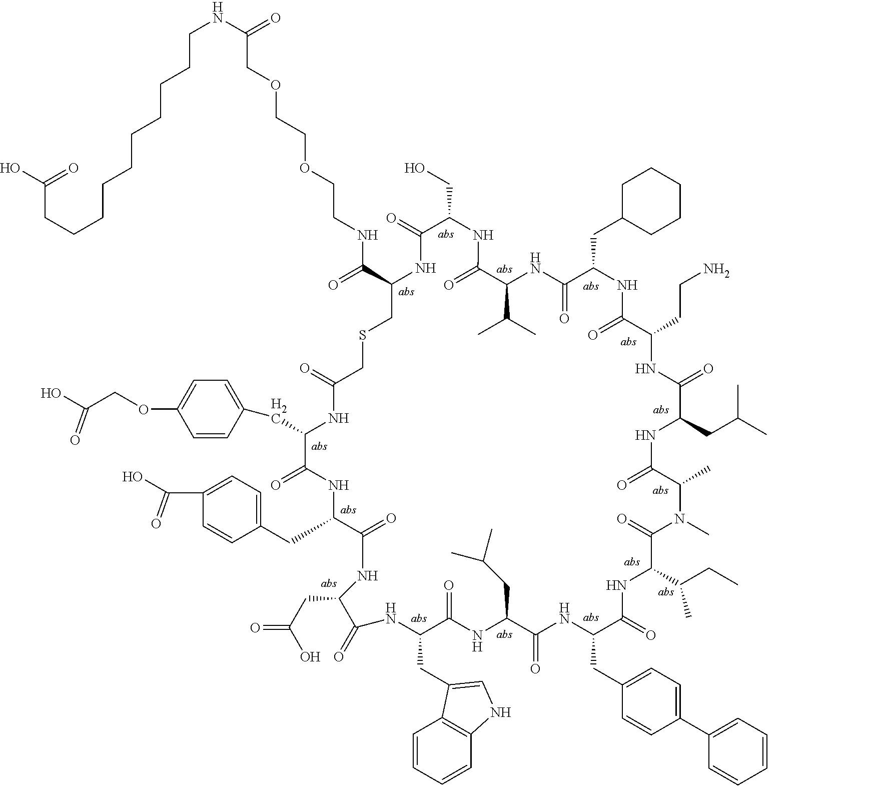

Example 1069 was prepared on a 50 μmol scale. The yield of the product was 14.7 mg, and its estimated purity by LCMS analysis was 94.7%. Analysis condition B: Retention time=2.04 min; ESI-MS(+) m/z [M+2H]²⁺: 1145.9.

### Preparation of Example 1070

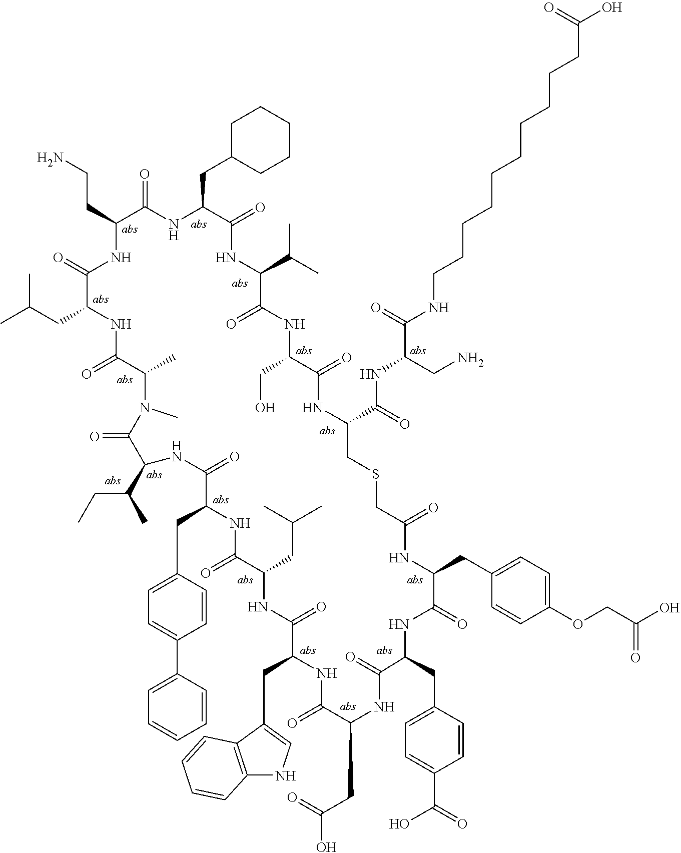

Example 1070 was prepared on a 50 μmol scale. The yield of the product was 7.5 mg, and its estimated purity by LCMS analysis was 91.5%. Analysis condition A: Retention time=1.58 min; ESI-MS(+) m/z [M+2H]²⁺: 1117.1.

### Preparation of Example 1071

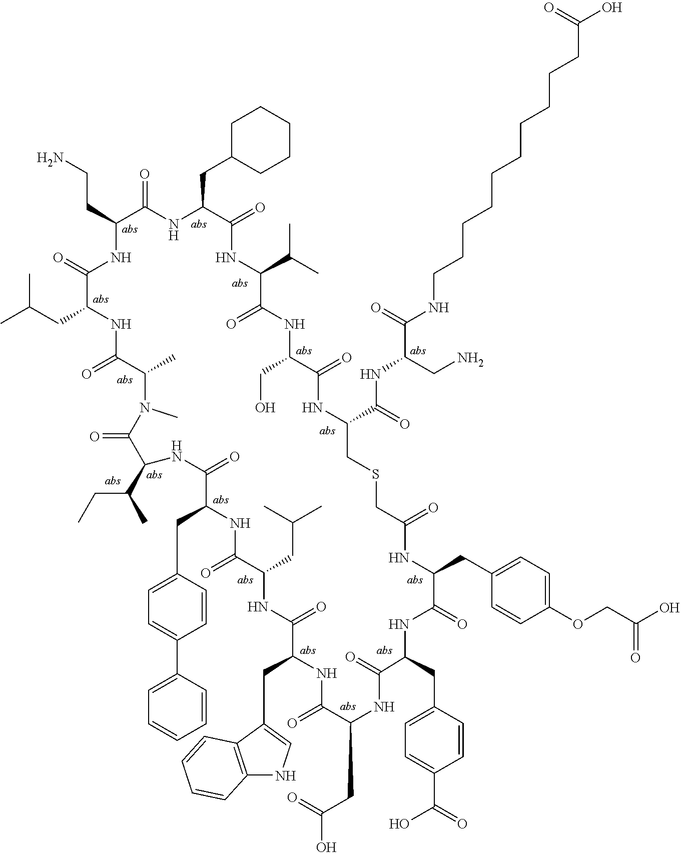

Example 1071 was prepared on a 50 μmol scale. The yield of the product was 7.7 mg, and its estimated purity by LCMS analysis was 93.6%. Analysis condition B: Retention time=1.75 min; ESI-MS(+) m/z [M+2H]²⁺: 1123.6.

### Preparation of Example 1072

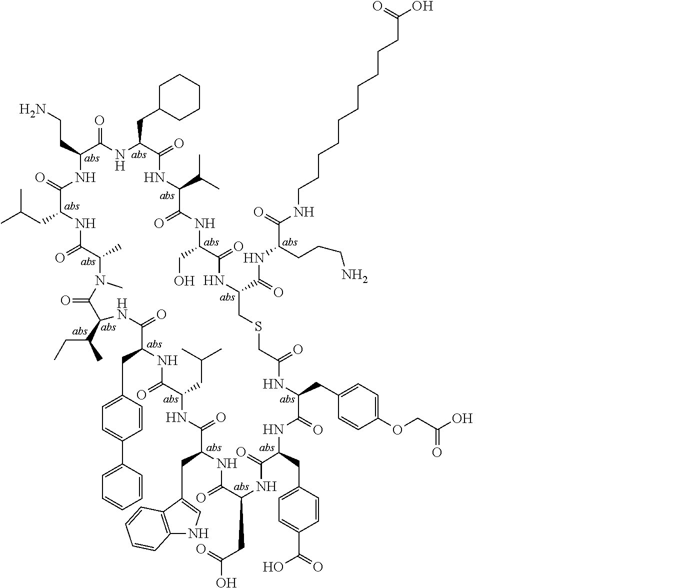

Example 1072 was prepared on a 50 μmol scale. The yield of the product was 6.8 mg, and its estimated purity by LCMS analysis was 96.2%. Analysis condition B: Retention time=1.71 min; ESI-MS(+) m/z [M+2H]²⁺: 1131.

### Preparation of Example 1073

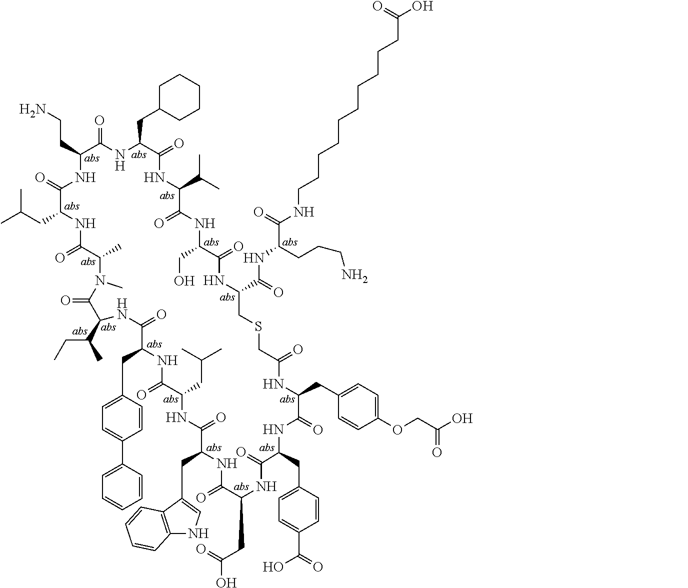

Example 1073 was prepared on a 50 μmol scale. The yield of the product was 8.6 mg, and its estimated purity by LCMS analysis was 94.6%. Analysis condition A: Retention time=1.49 min; ESI-MS(+) m/z [M+2H]²⁺: 1137.8.

### Preparation of Example 1074

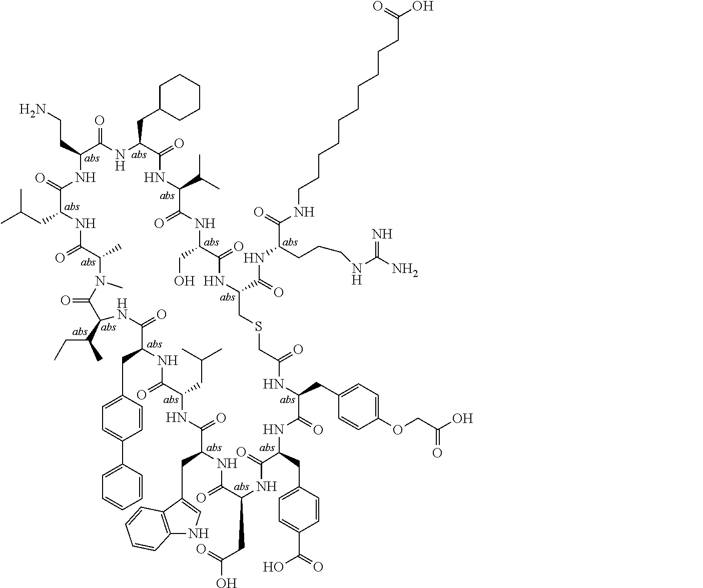

Example 1074 was prepared on a 50 μmol scale. The yield of the product was 3.7 mg, and its estimated purity by LCMS analysis was 89.9%. Analysis condition B: Retention time=1.83 min; ESI-MS(+) m/z [M+2H]²⁺: 1151.

### Preparation of Example 1075

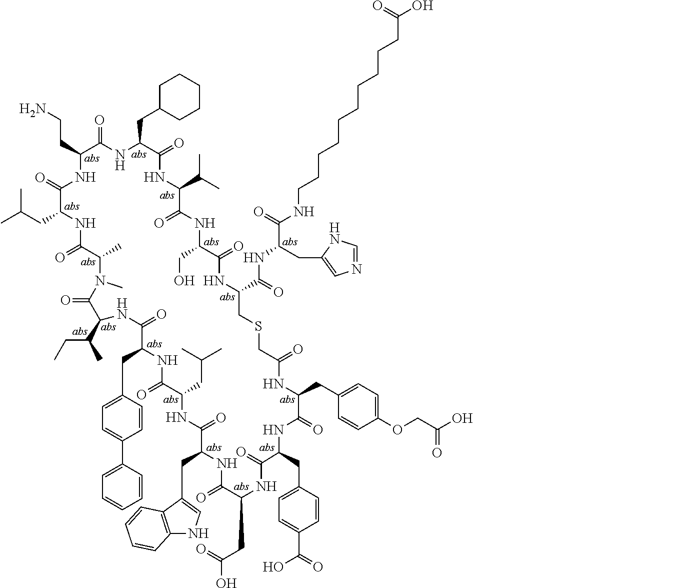

Example 1075 was prepared on a 50 μmol scale. The yield of the product was 14.7 mg, and its estimated purity by LCMS analysis was 90.9%. Analysis condition B: Retention time=1.77 min; ESI-MS(+) m/z [M+2H]²⁺: 1142.

### Preparation of Example 1076

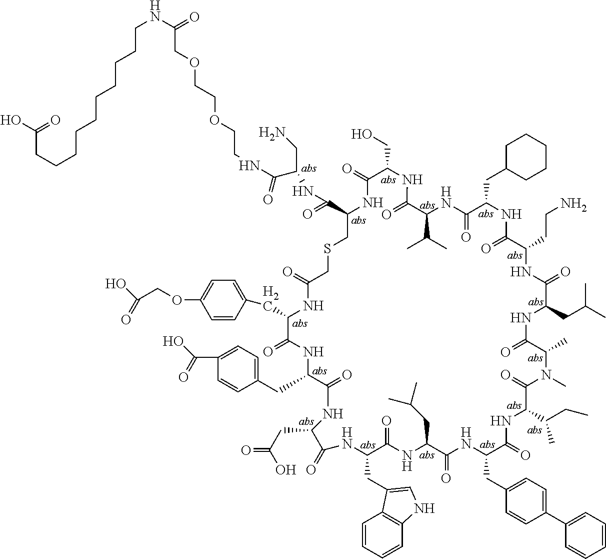 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1076 was prepared on a 50 μmol scale. The yield of the product was 23.1 mg, and its estimated purity by LCMS analysis was 84.1%. Analysis condition A: Retention time=1.59 min; ESI-MS(+) m/z [M+2H]²⁺: 1189.

### Preparation of Example 1077

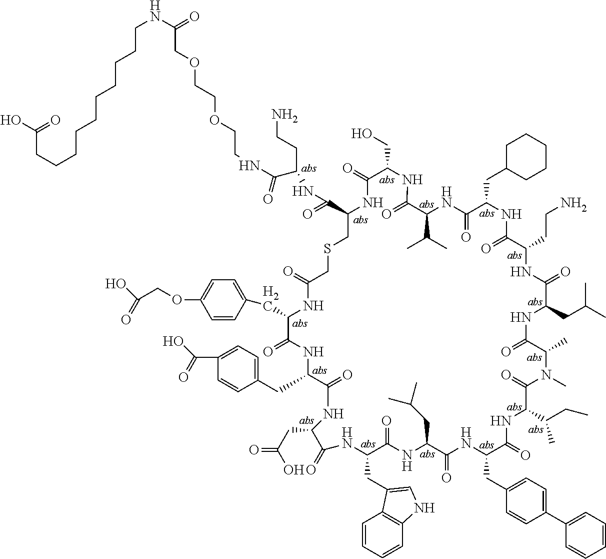 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1077 was prepared on a 50 μmol scale. The yield of the product was 28.8 mg, and its estimated purity by LCMS analysis was 100%. Analysis condition B: Retention time=1.79 min; ESI-MS(+) m/z [M+2H]²⁺: 1196.

### Preparation of Example 1078

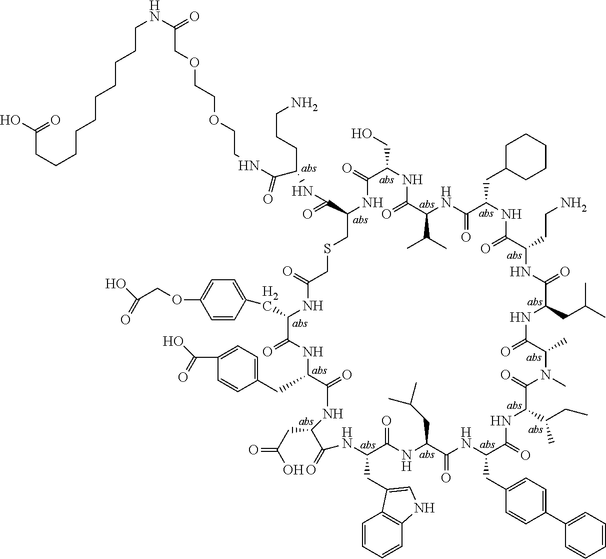 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1078 was prepared on a 50 μmol scale. The yield of the product was 12.5 mg, and its estimated purity by LCMS analysis was 99.4%. Analysis condition B: Retention time=1.89 min; ESI-MS(+) m/z [M+2H]²⁺: 1203.3.

### Preparation of Example 1079

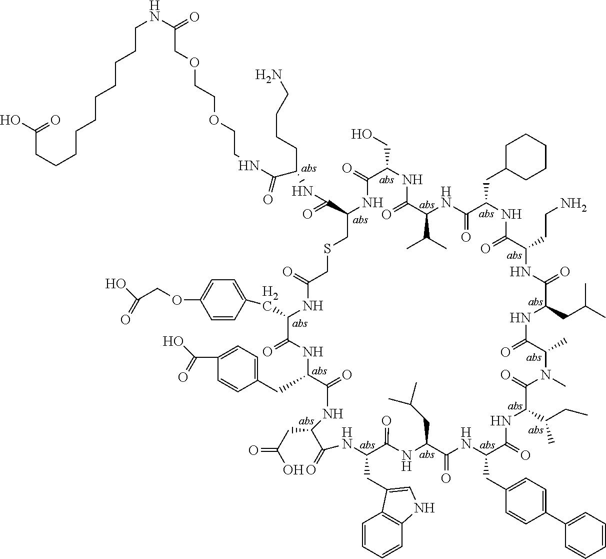 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1079 was prepared on a 50 μmol scale. The yield of the product was 17.4 mg, and its estimated purity by LCMS analysis was 92.4%. Analysis condition B: Retention time=1.8 min; ESI-MS(+) m/z [M+2H]²⁺: 1210.

### Preparation of Example 1080

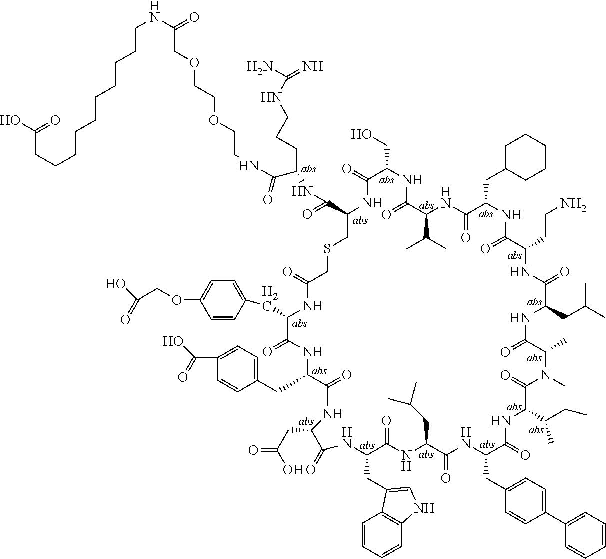 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCNC(=N)N)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1080 was prepared on a 50 μmol scale. The yield of the product was 10.3 mg, and its estimated purity by LCMS analysis was 98.2%. Analysis condition A: Retention time=1.62 min; ESI-MS(+) m/z [M+2H]²⁺: 1224.

### Preparation of Example 1081

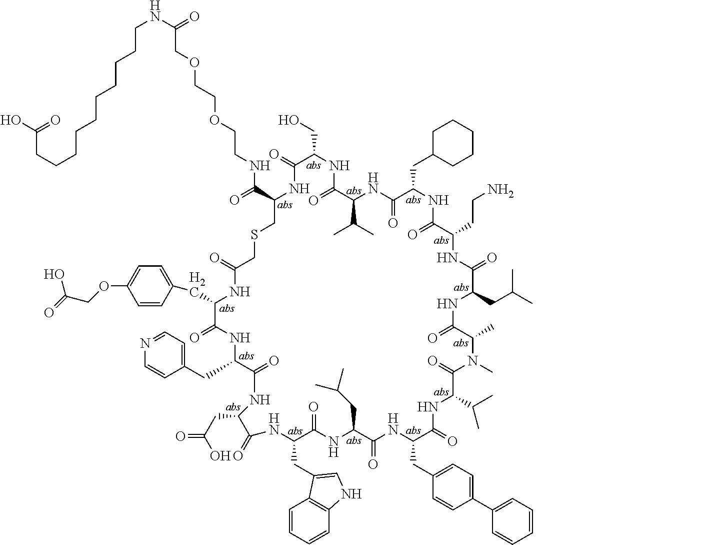 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1081 was prepared on a 50 μmol scale. The yield of the product was 29.9 mg, and its estimated purity by LCMS analysis was 100%. Analysis condition A: Retention time=1.77 min; ESI-MS(+) m/z [M+2H]²⁺: 1118.

### Preparation of Example 1082

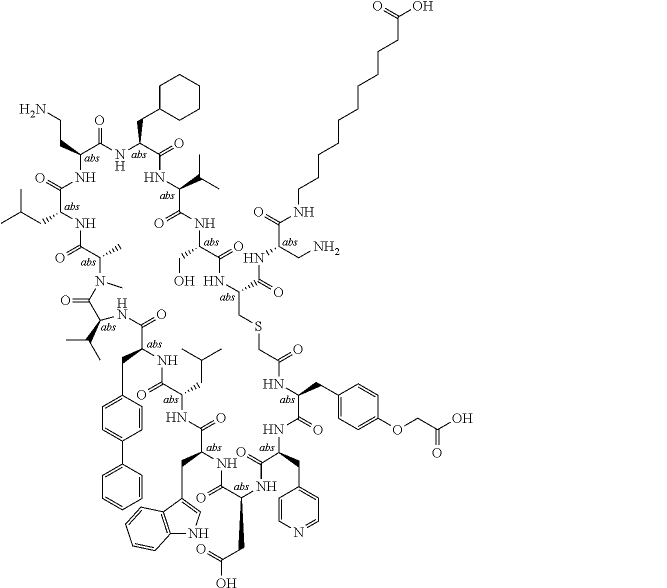

Example 1082 was prepared on a 50 μmol scale. The yield of the product was 3.1 mg, and its estimated purity by LCMS analysis was 94.8%. Analysis condition A: Retention time=1.75 min; ESI-MS(+) m/z [M+2H]²⁺: 1088.3.

### Preparation of Example 1083

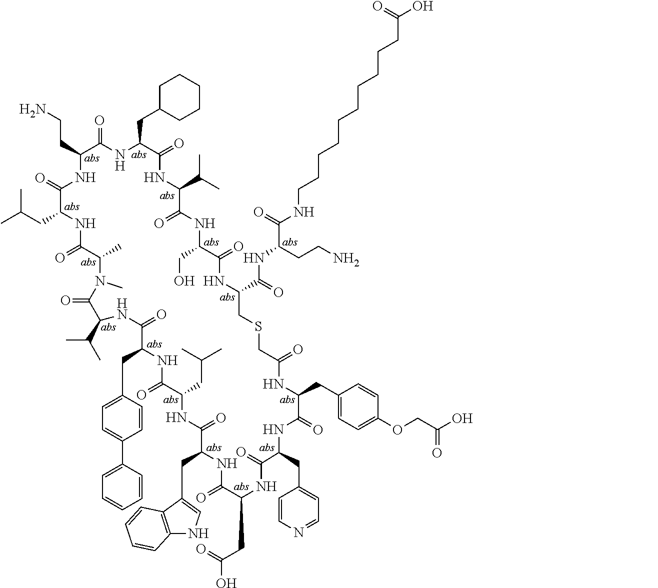

Example 1083 was prepared on a 50 μmol scale. The yield of the product was 6 mg, and its estimated purity by LCMS analysis was 89.4%. Analysis condition B: Retention time=1.64 min; ESI-MS(+) m/z [M+3H]³⁺: 730.4.

### Preparation of Example 1084

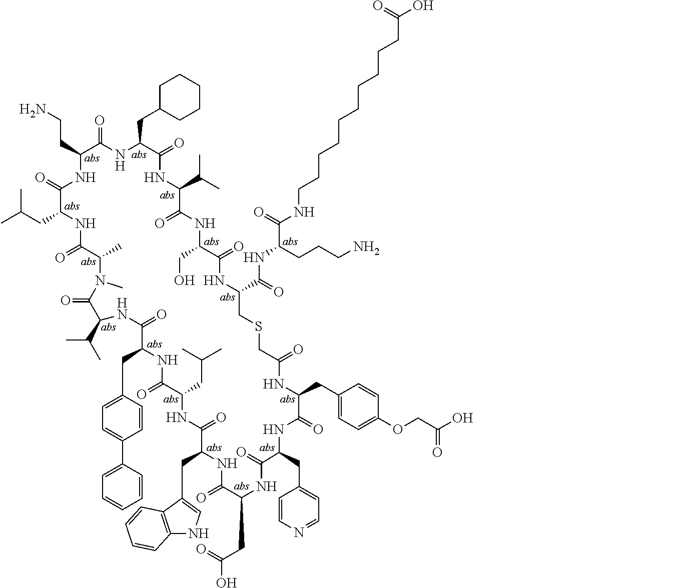

Example 1084 was prepared on a 50 μmol scale. The yield of the product was 11.9 mg, and its estimated purity by LCMS analysis was 95.7%. Analysis condition B: Retention time=1.71 min; ESI-MS(+) m/z [M+2H]²⁺: 1102.

### Preparation of Example 1085

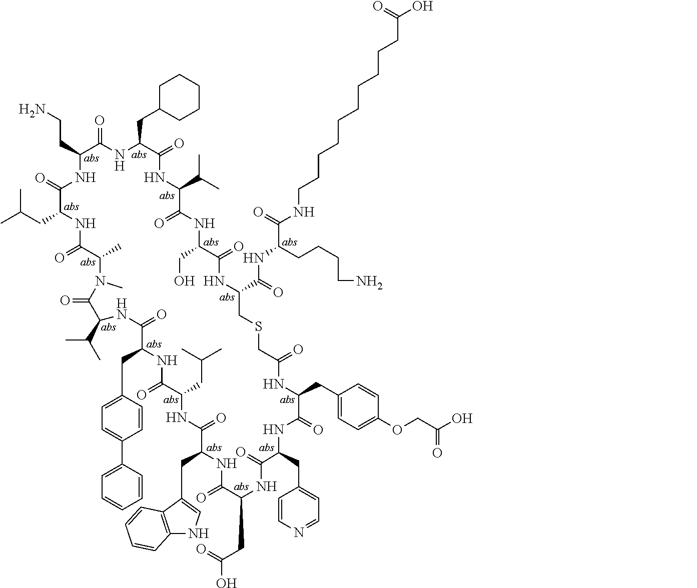

Example 1085 was prepared on a 50 μmol scale. The yield of the product was 6.5 mg, and its estimated purity by LCMS analysis was 96.2%. Analysis condition A: Retention time=1.7 min; ESI-MS(+) m/z [M+2H]²⁺: 1108.9.

### Preparation of Example 1086

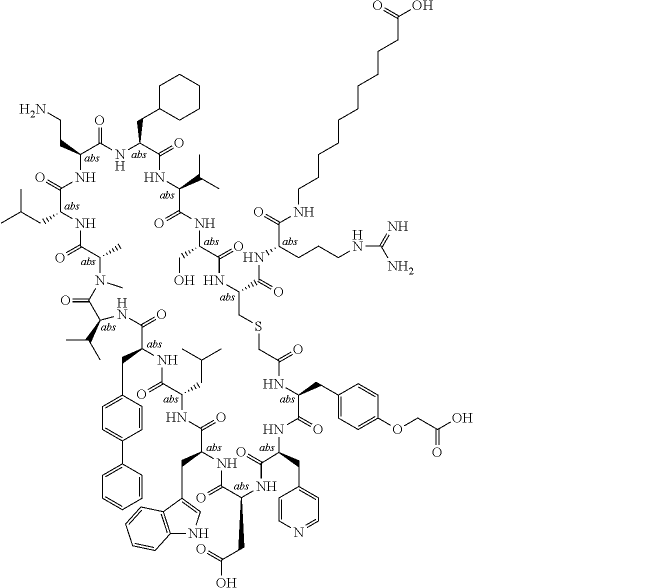

Example 1086 was prepared on a 50 μmol scale. The yield of the product was 5.4 mg, and its estimated purity by LCMS analysis was 86.7%. Analysis condition B: Retention time=1.63 min; ESI-MS(+) m/z [M+3H]³⁺: 749.2.

### Preparation of Example 1087

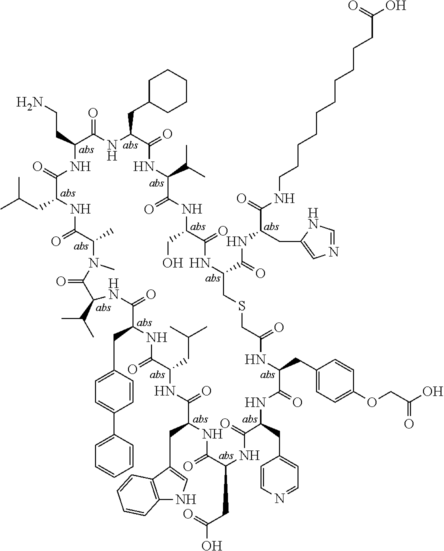

Example 1087 was prepared on a 50 μmol scale. The yield of the product was 6.6 mg, and its estimated purity by LCMS analysis was 89.5%. Analysis condition A: Retention time=1.64 min; ESI-MS(+) m/z [M+2H]²⁺: 1113.2.

### Preparation of Example 1088

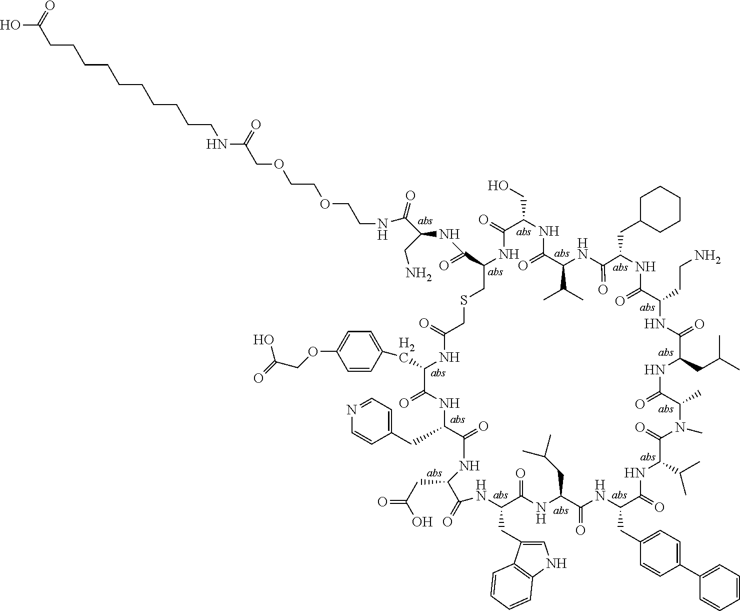 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1088 was prepared on a 50 μmol scale. The yield of the product was 13.3 mg, and its estimated purity by LCMS analysis was 93.2%. Analysis condition B: Retention time=1.65 min; ESI-MS(+) m/z [M+2H]²⁺: 1160.8.

### Preparation of Example 1089

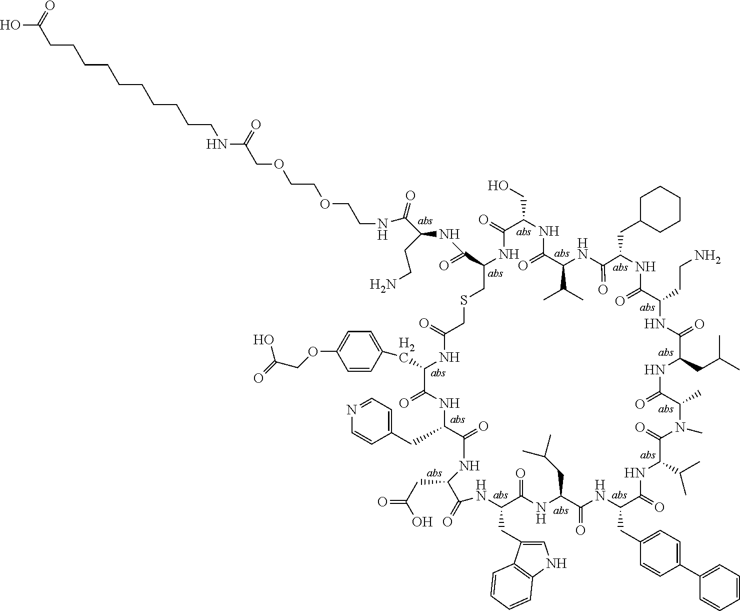 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1089 was prepared on a 50 μmol scale. The yield of the product was 18 mg, and its estimated purity by LCMS analysis was 88.5%. Analysis condition B: Retention time=1.75 min; ESI-MS(+) m/z [M+3H]³⁺: 778.9.

### Preparation of Example 1090

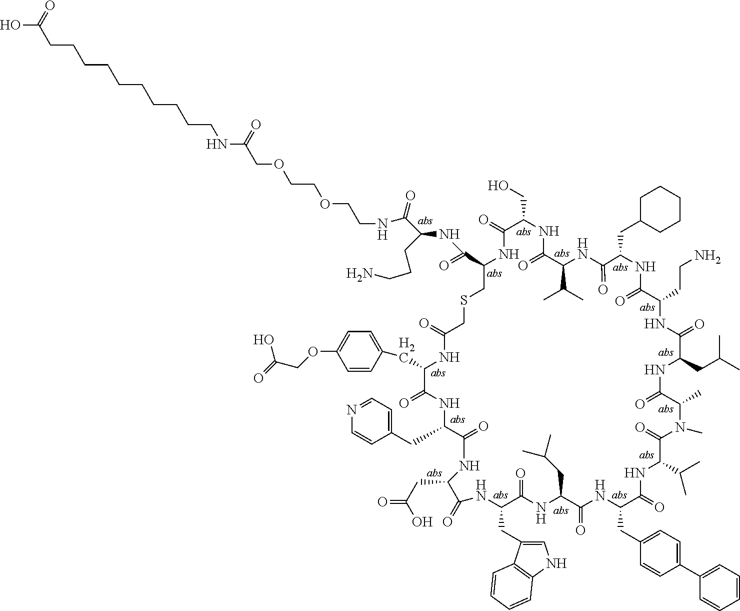 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1090 was prepared on a 50 μmol scale. The yield of the product was 8.5 mg, and its estimated purity by LCMS analysis was 87.1%. Analysis condition B: Retention time=1.64 min; ESI-MS(+) m/z [M+3H]³⁺: 783.4.

### Preparation of Example 1091

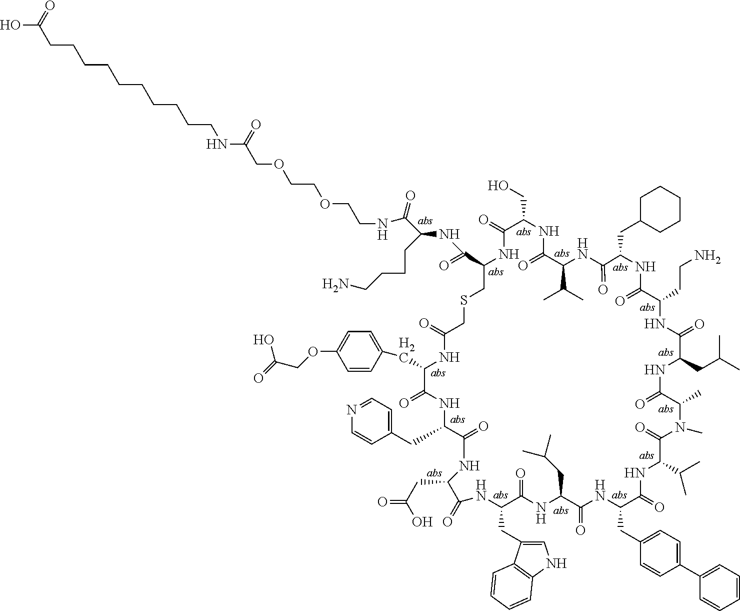 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1091 was prepared on a 50 μmol scale. The yield of the product was 12.6 mg, and its estimated purity by LCMS analysis was 87.2%. Analysis condition B: Retention time=1.65 min; ESI-MS(+) m/z [M+3H]³⁺: 788.2.

### Preparation of Example 1092

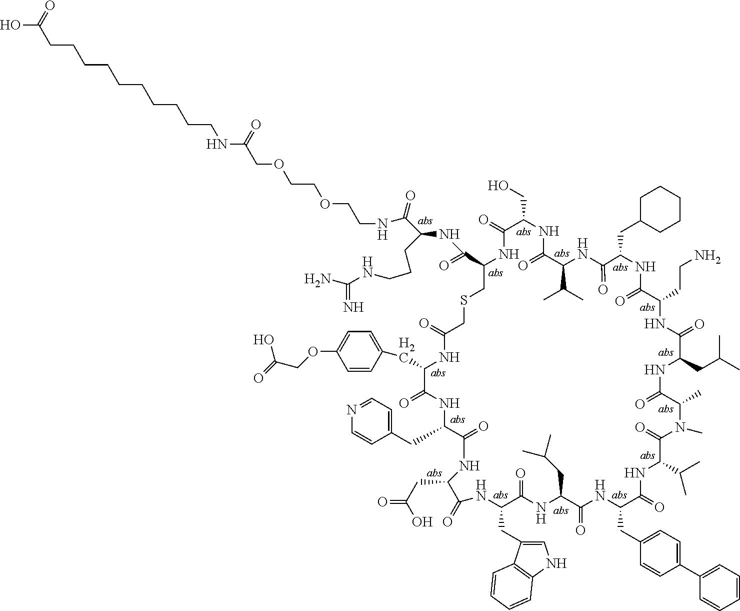 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCNC(=N)N)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1092 was prepared on a 50 μmol scale. The yield of the product was 6.5 mg, and its estimated purity by LCMS analysis was 91.4%. Analysis condition A: Retention time=1.66 min; ESI-MS(+) m/z [M+3H]³⁺: 798.

### Preparation of Example 1093

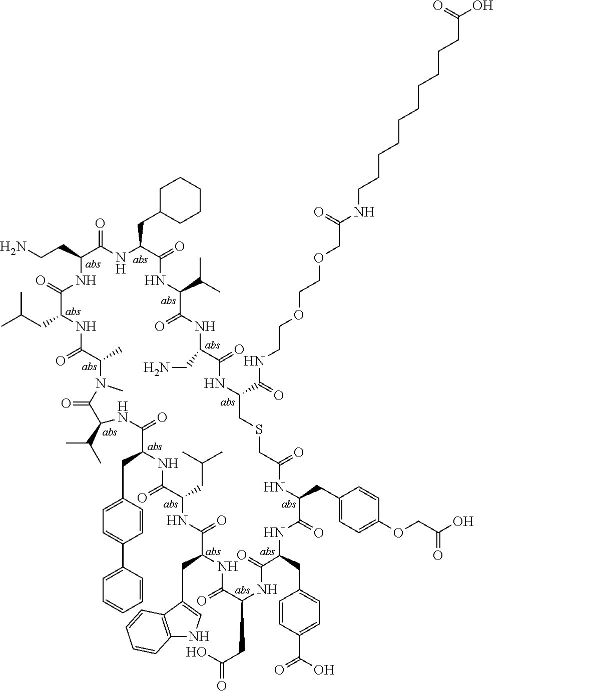

Example 1093 was prepared on a 50 μmol scale. The yield of the product was 29.8 mg, and its estimated purity by LCMS analysis was 95.2%. Analysis condition A: Retention time=1.8 min; ESI-MS(+) m/z [M+2H]²⁺: 1139.2.

### Preparation of Example 1094

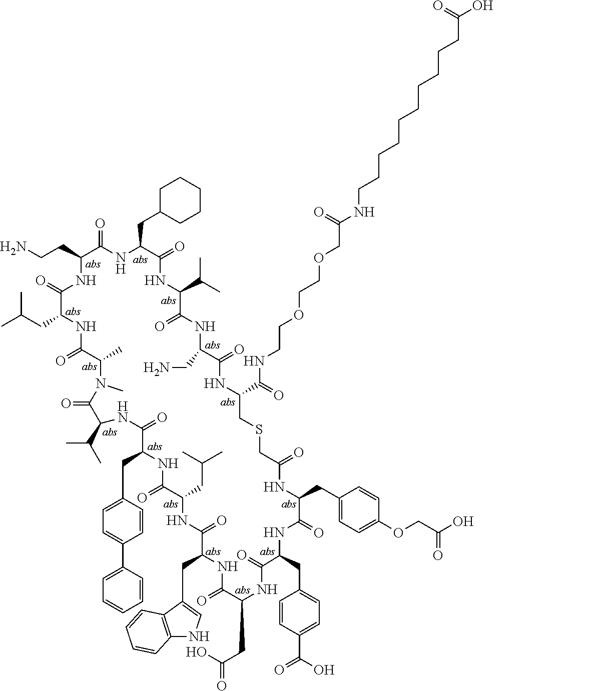

Example 1094 was prepared on a 50 μmol scale. The yield of the product was 23.5 mg, and its estimated purity by LCMS analysis was 95.9%. Analysis condition A: Retention time=1.53, 1.59 min; ESI-MS(+) m/z [M+3H]³⁺: 764.18, 764.18.

### Preparation of Example 1095

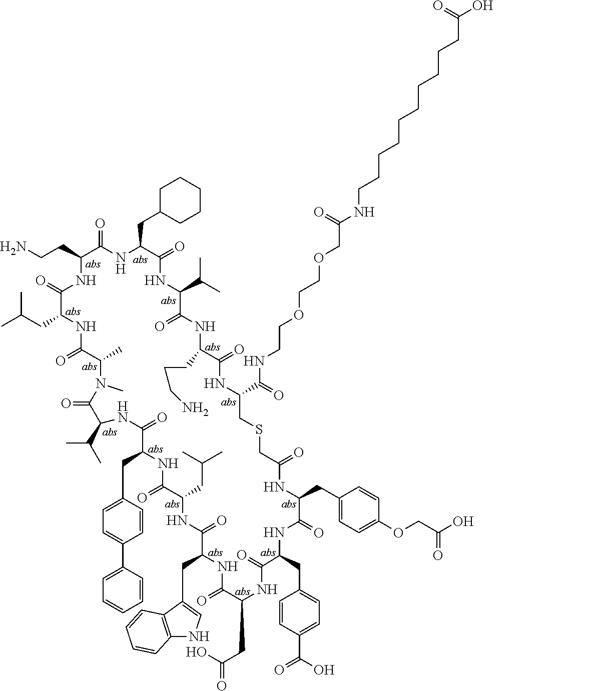

Example 1095 was prepared on a 50 μmol scale. The yield of the product was 18 mg, and its estimated purity by LCMS analysis was 95.5%. Analysis condition A: Retention time=1.58 min; ESI-MS(+) m/z [M+3H]³⁺: 766.9.

### Preparation of Example 1096

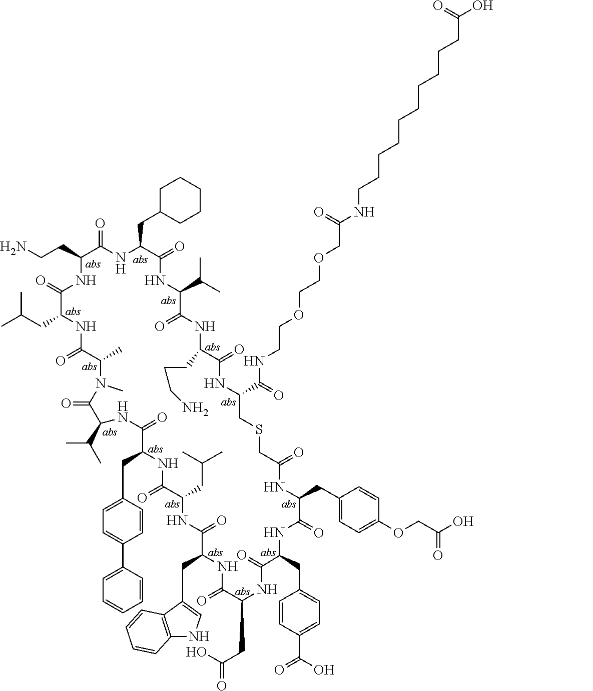

Example 1096 was prepared on a 50 μmol scale. The yield of the product was 26.1 mg, and its estimated purity by LCMS analysis was 96.5%. Analysis condition A: Retention time=1.74 min; ESI-MS(+) m/z [M+2H]²⁺: 1160.2.

### Preparation of Example 1097

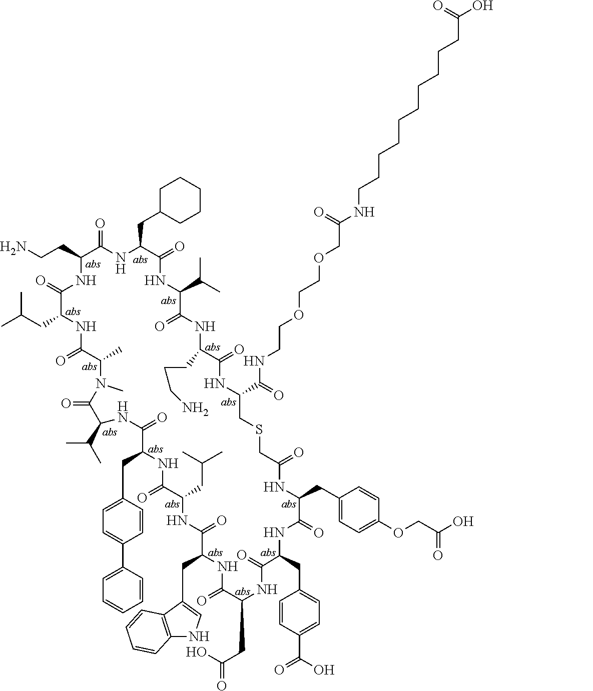

Example 1097 was prepared on a 50 μmol scale. The yield of the product was 37.8 mg, and its estimated purity by LCMS analysis was 100%. Analysis condition A: Retention time=1.63 min; ESI-MS(+) m/z [M+2H]²⁺: 1173.9.

### Preparation of Example 1098

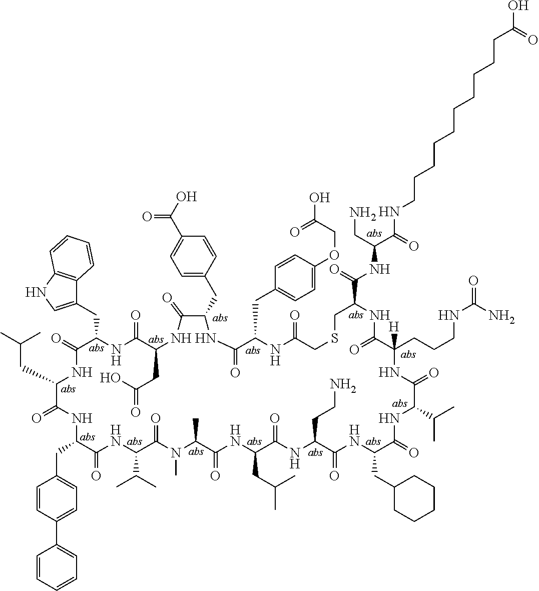 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1098 was prepared on a 50 μmol scale. The yield of the product was 13.6 mg, and its estimated purity by LCMS analysis was 90.2%. Analysis condition B: Retention time=1.66 min; ESI-MS(+) m/z [M+2H]²⁺: 1145.2.

### Preparation of Example 1099

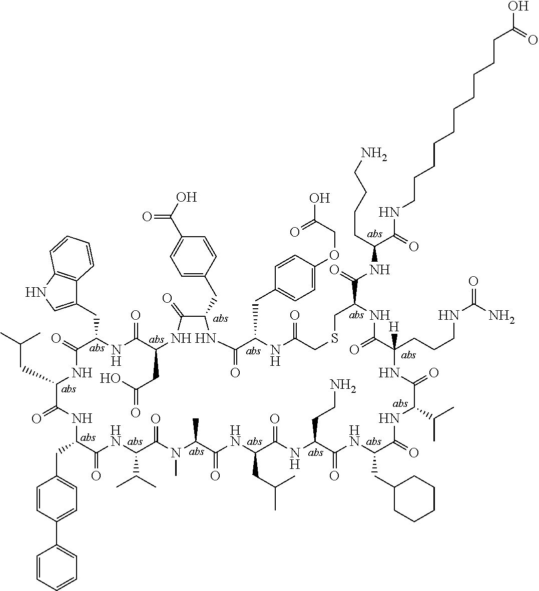 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1099 was prepared on a 50 μmol scale. The yield of the product was 16.7 mg, and its estimated purity by LCMS analysis was 88.1%. Analysis condition B: Retention time=1.62 min; ESI-MS(+) m/z [M+2H]²⁺: 1165.9.

### Preparation of Example 1100

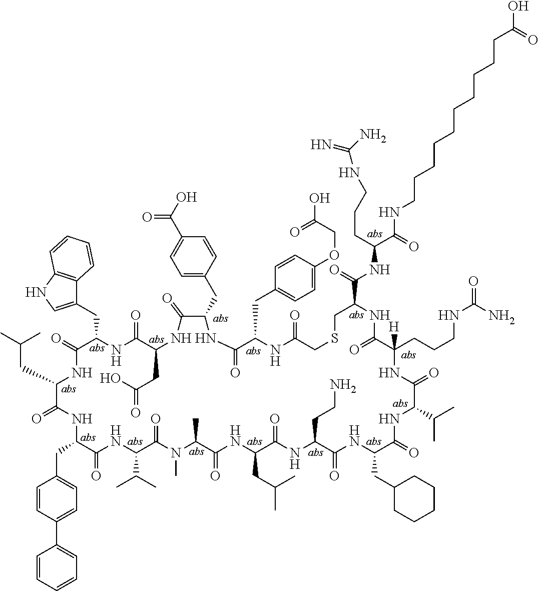 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCNC(=N)N)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1100 was prepared on a 50 μmol scale. The yield of the product was 20.3 mg, and its estimated purity by LCMS analysis was 88%. Analysis condition A: Retention time=1.68 min; ESI-MS(+) m/z [M+2H]²⁺: 1180.

### Preparation of Example 1101

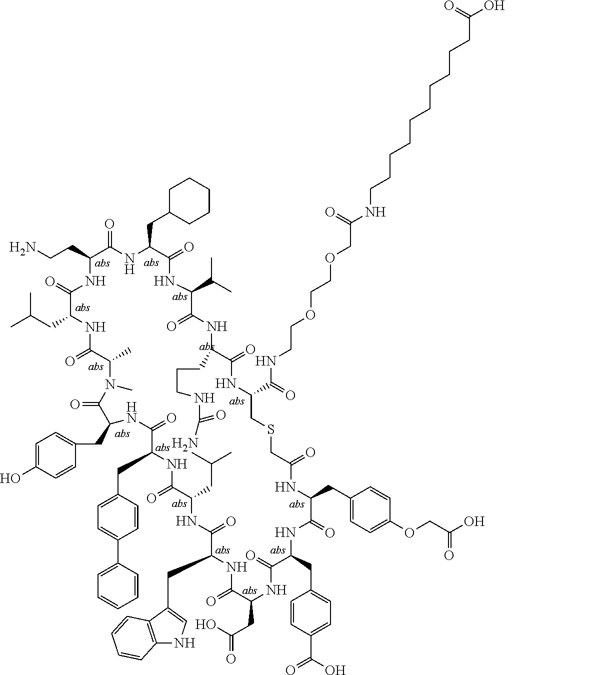 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(O)cc2)NC(=O)[C@H](Cc2ccc(-c3ccccc3)cc2)NC1=O]

Example 1101 was prepared on a 50 μmol scale. The yield of the product was 45.1 mg, and its estimated purity by LCMS analysis was 95.1%. Analysis condition A: Retention time=1.7 min; ESI-MS(+) m/z [M+2H]²⁺: 1206.1.

### Preparation of Example 1102

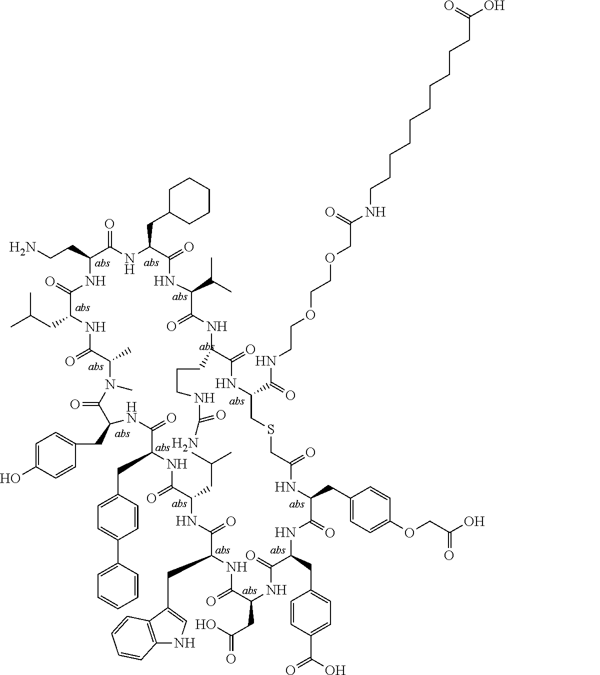 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(O)cc2)NC(=O)[C@H](Cc2ccc(-c3ccccc3)cc2)NC1=O]

Example 1102 was prepared on a 50 μmol scale. The yield of the product was 14.9 mg, and its estimated purity by LCMS analysis was 97.3%. Analysis condition B: Retention time=1.78 min; ESI-MS(+) m/z [M+2H]²⁺: 1199.2.

### Preparation of Example 1103

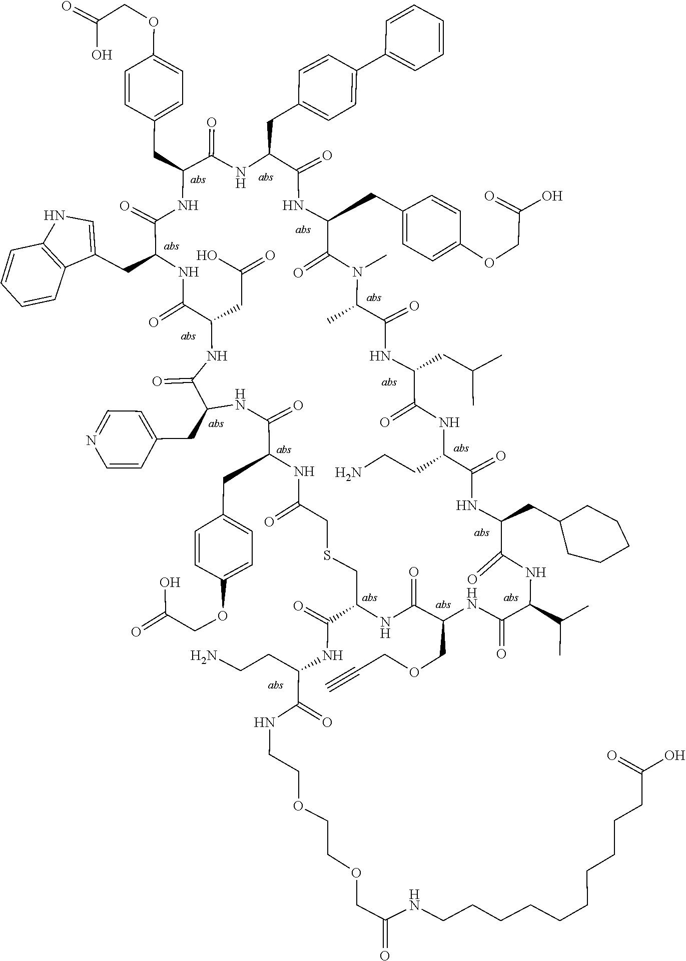

Example 1103 was prepared on a 50 μmol scale. The yield of the product was 17 mg, and its estimated purity by LCMS analysis was 94.7%. Analysis condition B: Retention time=1.71 min; ESI-MS(+) m/z [M+2H]²⁺: 1301.9.

### Preparation of Example 1104

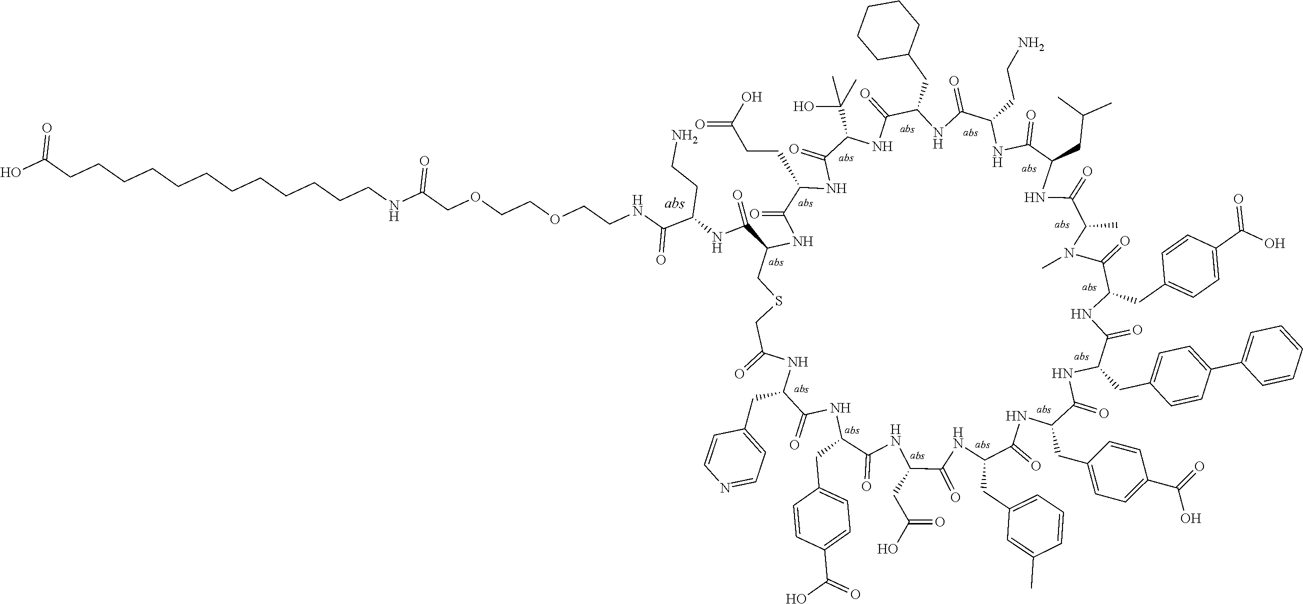 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 1104 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 19% B, 19-59% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 79.7 mg, and its estimated purity by LCMS analysis was 91.5%. Analysis condition A: Retention time=min; ESI-MS(+) m/z [M+3H]³⁺: 845.9.

### Preparation of Example 1105

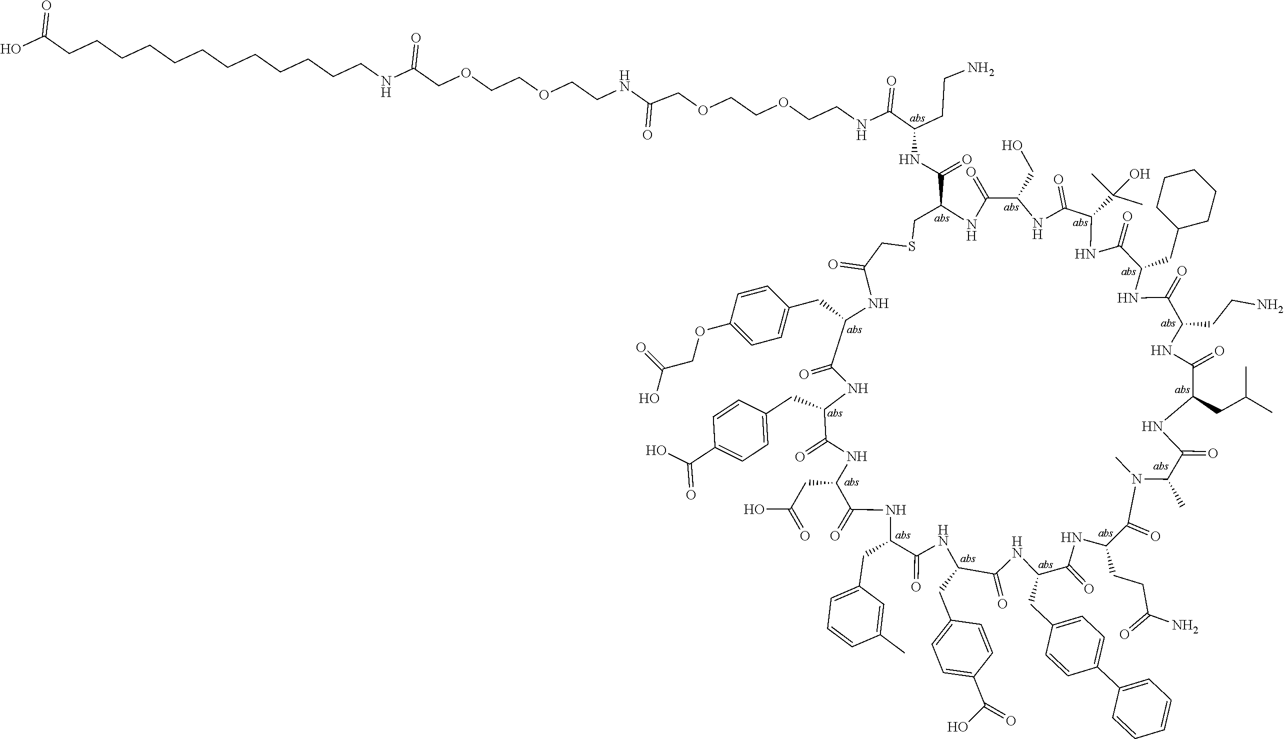 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](Cc3ccc(C(=O)O)cc3)NC2=O)c1]

Example 1105 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 30.1 mg, and its estimated purity by LCMS analysis was 90.3%. Analysis condition A: Retention time=1.5 min; ESI-MS(+) m/z [M+2H]²⁺: 1324.5.

### Preparation of Example 1106

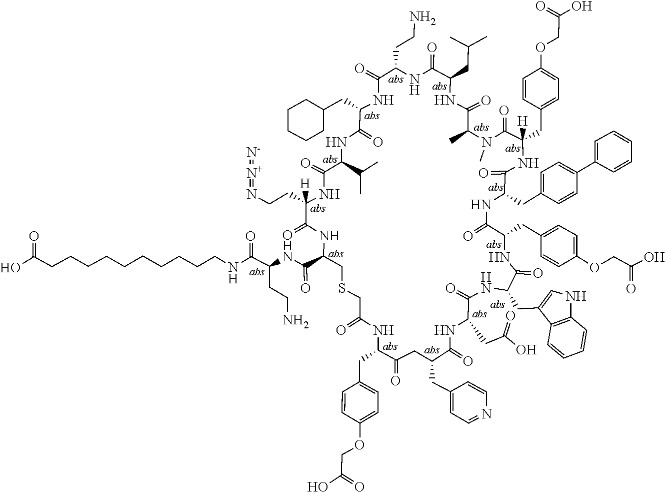 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)CC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCN=[N+]=[N-])NC(=O)[C@H](C(C)C)NC(=O)[C@H](CC2CCCCC2)NC(=O)[C@H](CCN)NC1=O]

Example 1106 was prepared on a 50 μmol scale. The yield of the product was 17.5 mg, and its estimated purity by LCMS analysis was 95.1%. Analysis condition: Retention time=1.77 min; ESI-MS(+) m/z [M+2H]²⁺: 1229.3.

### Preparation of Example 1107

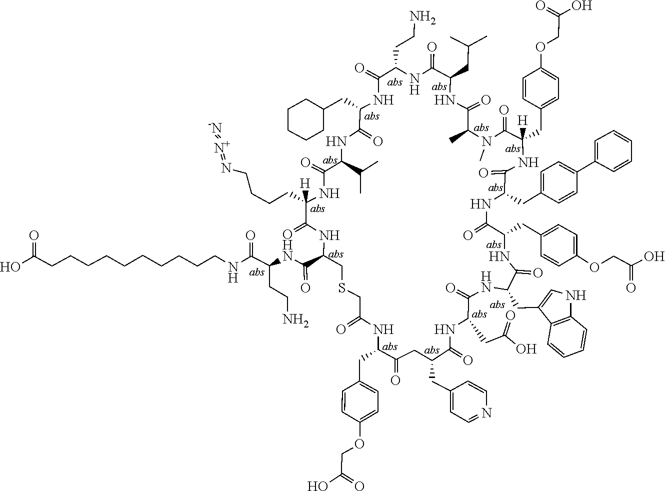

Example 1107 was prepared on a 50 μmol scale. The yield of the product was 9.4 mg, and its estimated purity by LCMS analysis was 90.1%. Analysis condition B: Retention time=1.76 min; ESI-MS(+) m/z [M+2H]²⁺: 1243.

### Preparation of Example 1108

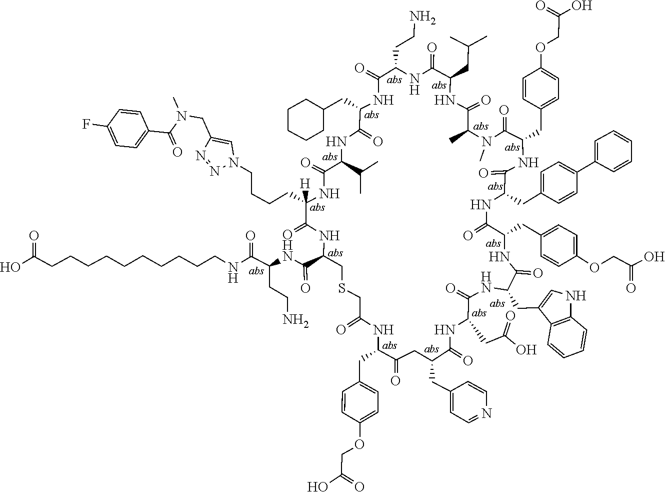 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)CC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCCn2cc(CN(C)C(=O)c3ccc(F)cc3)nn2)NC(=O)[C@H](C(C)C)NC(=O)[C@H](CC2CCCCC2)NC(=O)[C@H](CCN)NC1=O]

Example 1108 was prepared on a 3.5 μmol scale. The yield of the product was 2.1 mg, and its estimated purity by LCMS analysis was 92.1%. Analysis condition: Retention time=1.57 min; ESI-MS(+) m/z [M+2H]²⁺: 1339.1.

### Preparation of Example 1109

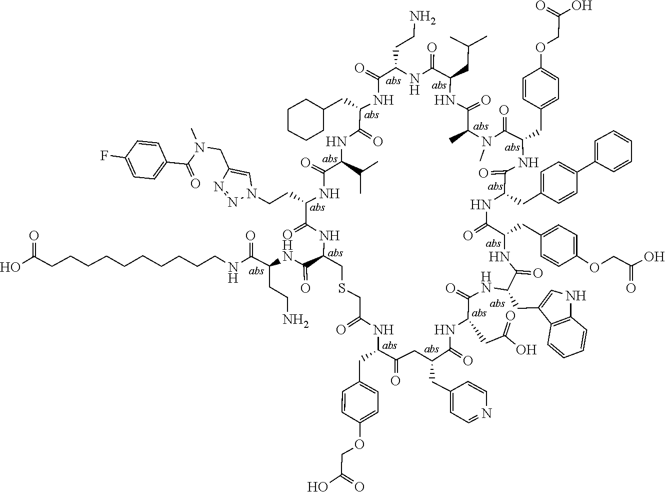 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)CC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCn2cc(CN(C)C(=O)c3ccc(F)cc3)nn2)NC(=O)[C@H](C(C)C)NC(=O)[C@H](CC2CCCCC2)NC(=O)[C@H](CCN)NC1=O]

Example 1109 was prepared on a 6.1 μmol scale. The yield of the product was 6.6 mg, and its estimated purity by LCMS analysis was 92.2%. Analysis condition: Retention time=1.52, 1.56 min; ESI-MS(+) m/z [M+2H]²⁺: 1325.2.

### Preparation of Example 1110

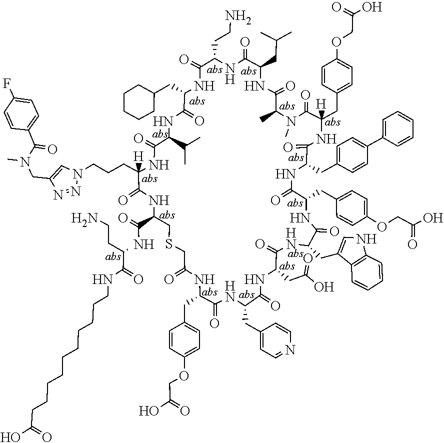

Example 1110 was prepared on a 2 μmol scale. The yield of the product was 1.8 mg, and its estimated purity by LCMS analysis was 87.5%. Analysis condition A: Retention time=1.55 min; ESI-MS(+) m/z [M+2H]²⁺: 1332.1.

### Preparation of Example 1111

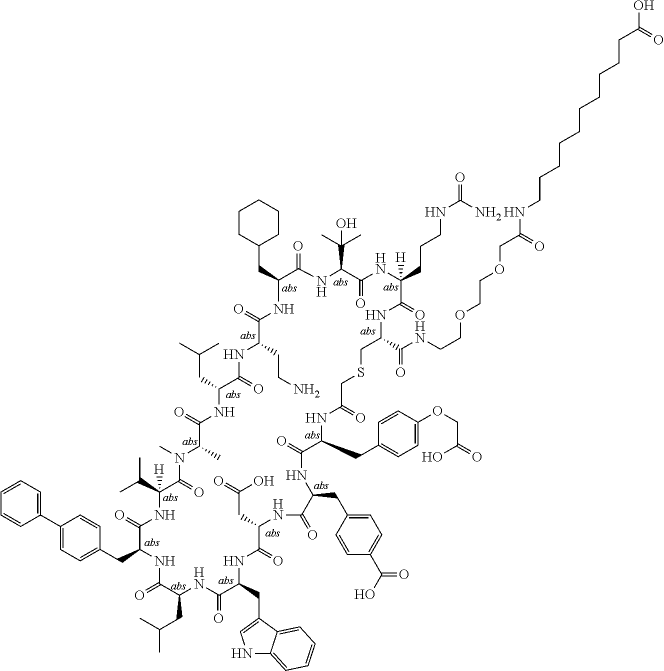 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1111 was prepared on a 50 μmol scale. The yield of the product was 15.8 mg, and its estimated purity by LCMS analysis was 95.8%. Analysis condition B: Retention time=1.93 min; ESI-MS(+) m/z [M+2H]²⁺: 1182.1.

### Preparation of Example 1112

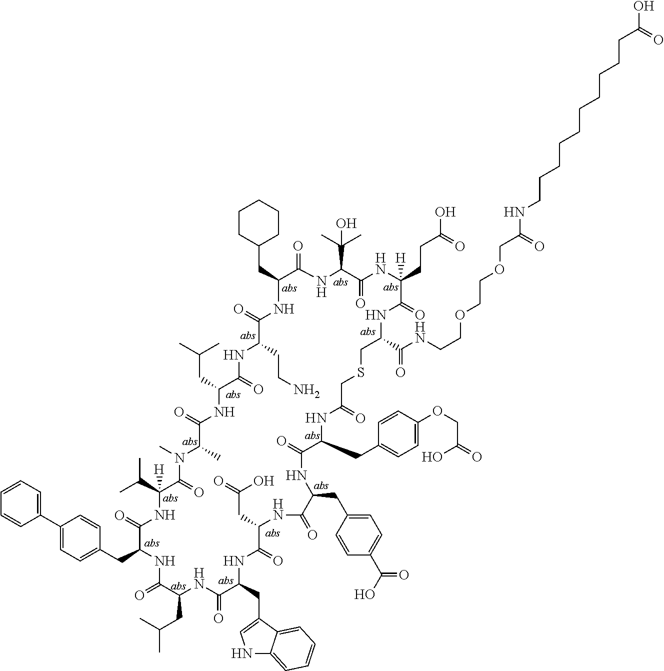 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1112 was prepared on a 50 μmol scale. The yield of the product was 26.7 mg, and its estimated purity by LCMS analysis was 96.6%. Analysis condition B: Retention time=1.95 min; ESI-MS(+) m/z [M+2H]²⁺: 1168.1.

### Preparation of Example 1113

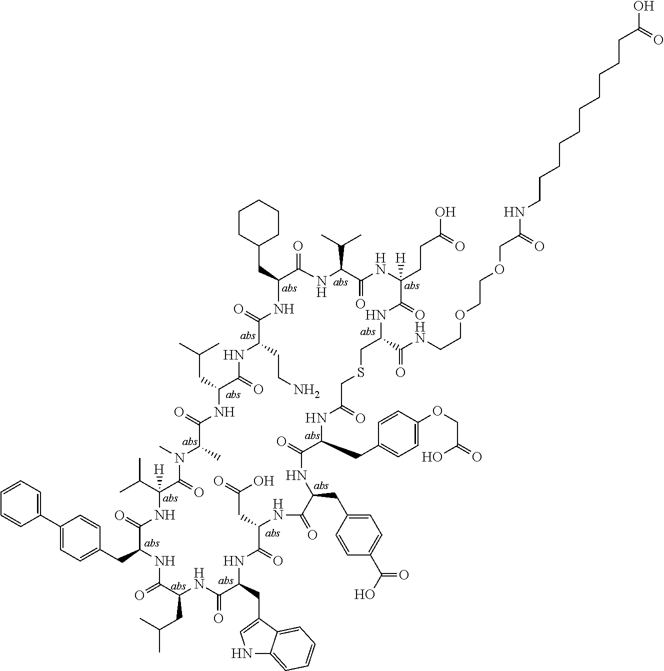 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1113 was prepared on a 50 μmol scale. The yield of the product was 15.1 mg, and its estimated purity by LCMS analysis was 87.6%. Analysis condition B: Retention time=1.94 min; ESI-MS(+) m/z [M+2H]²⁺: 1160.1.

### Preparation of Example 1114

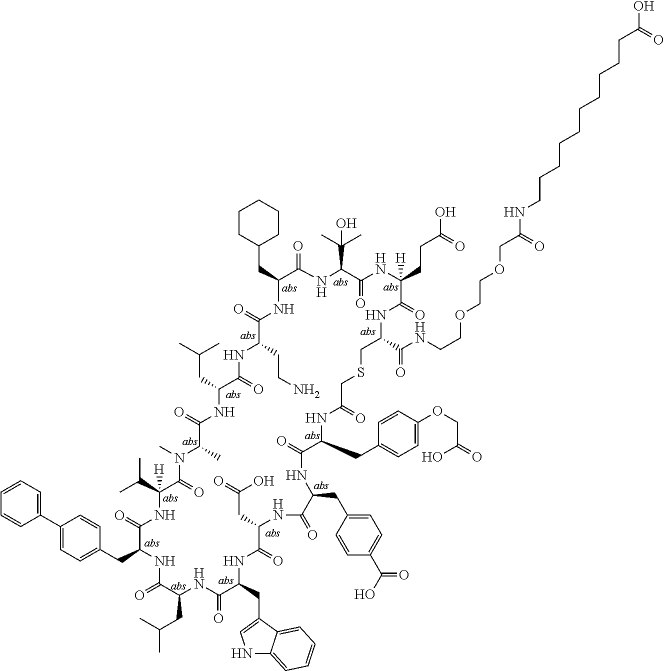 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1114 was prepared on a 50 μmol scale. The yield of the product was 20.3 mg, and its estimated purity by LCMS analysis was 95.9%. Analysis condition A: Retention time=1.63 min; ESI-MS(+) m/z [M+2H]²⁺: 1167.2.

### Preparation of Example 1115

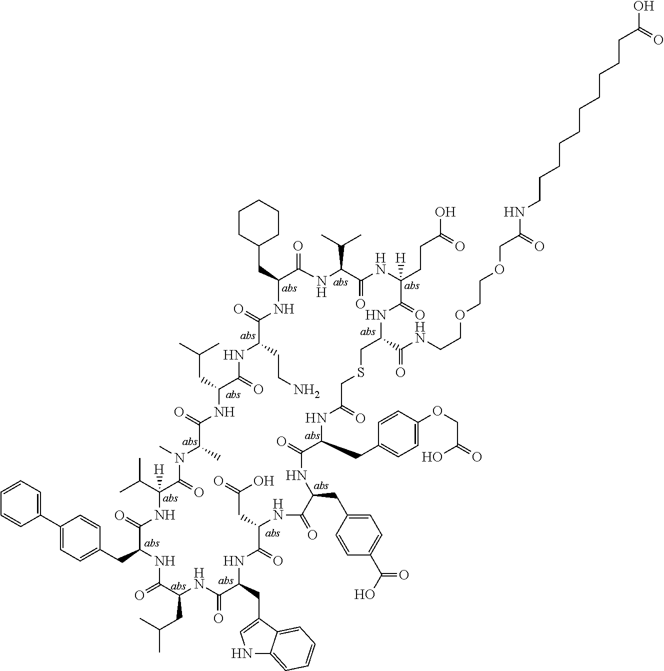 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1115 was prepared on a 50 μmol scale. The yield of the product was 1.5 mg, and its estimated purity by LCMS analysis was 100%. Analysis condition B: Retention time=2.08 min; ESI-MS(+) m/z [M+2H]²⁺: 1160.

### Preparation of Example 1116

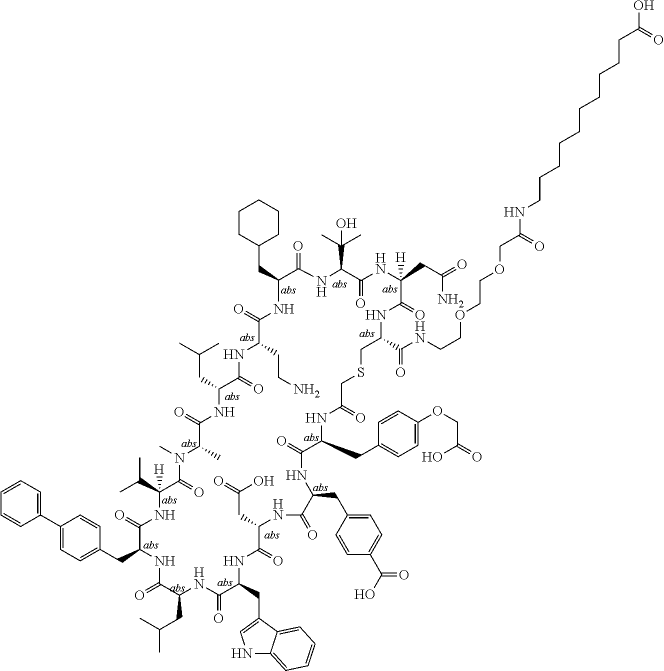 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1116 was prepared on a 50 μmol scale. The yield of the product was 16.2 mg, and its estimated purity by LCMS analysis was 95.4%. Analysis condition B: Retention time=1.96 min; ESI-MS(+) m/z [M+2H]²⁺: 1160.6.

### Preparation of Example 1117

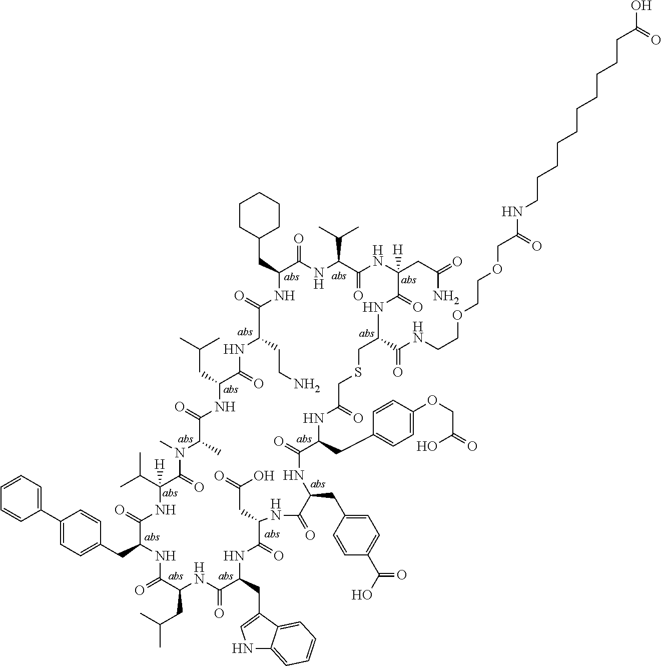 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1117 was prepared on a 50 μmol scale. The yield of the product was 25.1 mg, and its estimated purity by LCMS analysis was 96.5%. Analysis condition B: Retention time=1.96 min; ESI-MS(+) m/z [M+2H]²⁺: 1152.1.

### Preparation of Example 1118

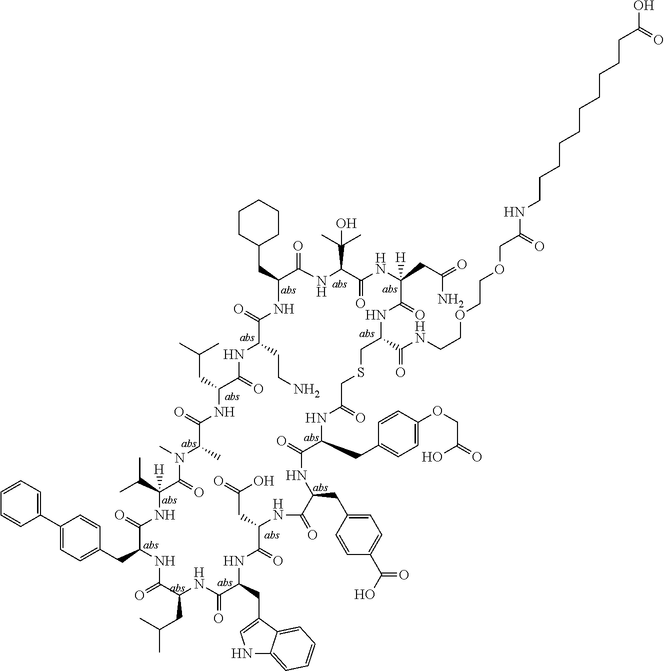 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1118 was prepared on a 50 μmol scale. The yield of the product was 26.3 mg, and its estimated purity by LCMS analysis was 96.4%. Analysis condition B: Retention time=2.11 min; ESI-MS(+) m/z [M+2H]²⁺: 1161.1.

### Preparation of Example 1119

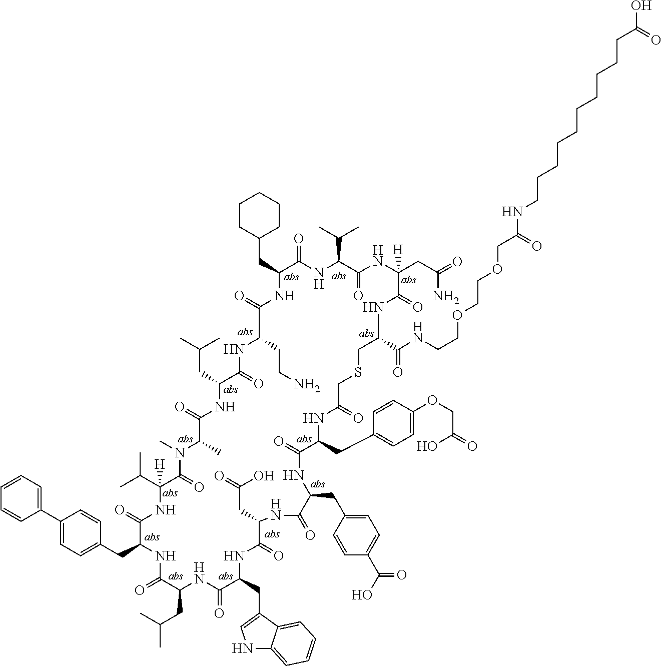 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1119 was prepared on a 50 μmol scale. The yield of the product was 27.7 mg, and its estimated purity by LCMS analysis was 98.1%. Analysis condition B: Retention time=1.98 min; ESI-MS(+) m/z [M+2H]²⁺: 1153.1.

### Preparation of Example 1120

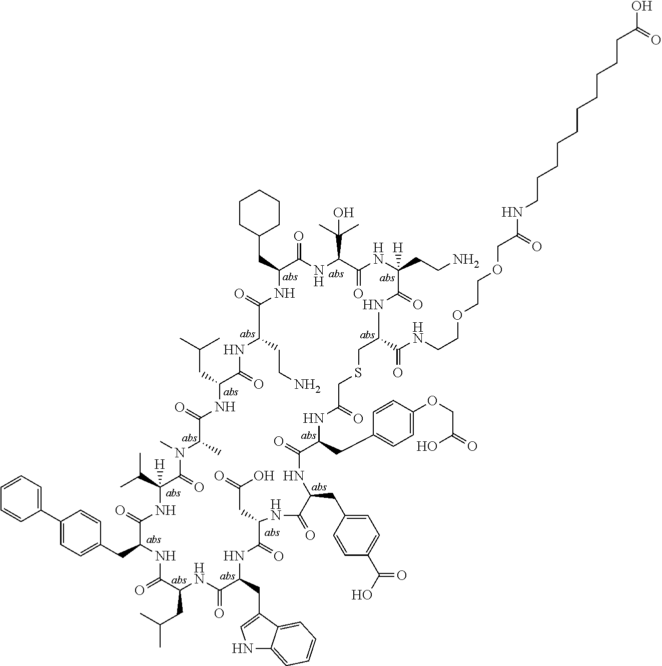 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCN)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1120 was prepared on a 50 μmol scale. The yield of the product was 16.8 mg, and its estimated purity by LCMS analysis was 97.4%. Analysis condition A: Retention time=1.65 min; ESI-MS(+) m/z [M+2H]²⁺: 1153.1.

### Preparation of Example 1121

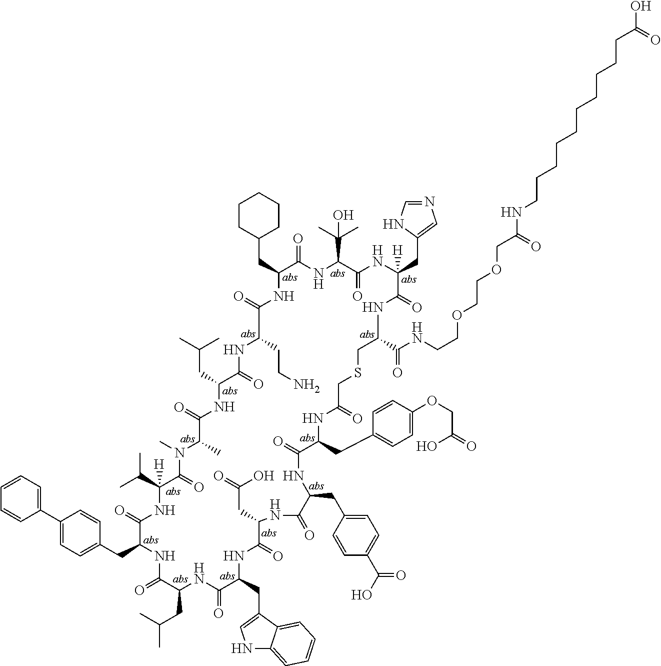 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1121 was prepared on a 50 μmol scale. The yield of the product was 22.4 mg, and its estimated purity by LCMS analysis was 96.4%. Analysis condition B: Retention time=1.94 min; ESI-MS(+) m/z [M+2H]²⁺: 1172.1.

### Preparation of Example 1122

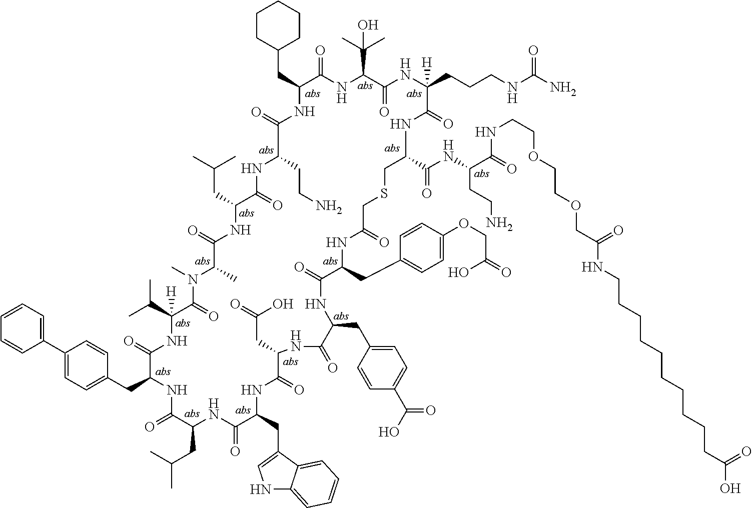 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCNC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1122 was prepared on a 50 μmol scale. The yield of the product was 14 mg, and its estimated purity by LCMS analysis was 99%. Analysis condition B: Retention time=1.95 min; ESI-MS(+) m/z [M+2H]²⁺: 1232.1.

### Preparation of Example 1123

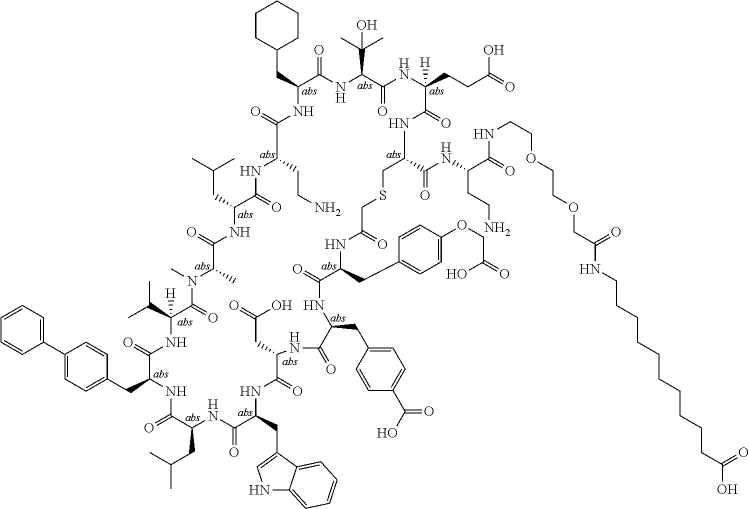 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1123 was prepared on a 50 μmol scale. The yield of the product was 15.2 mg, and its estimated purity by LCMS analysis was 95.8%. Analysis condition A: Retention time=1.58 min; ESI-MS(+) m/z [M+2H]²⁺: 1218.

### Preparation of Example 1124

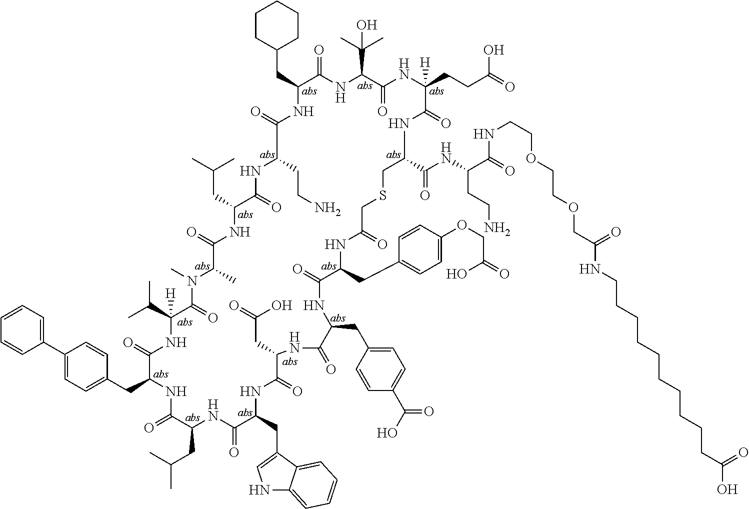 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1124 was prepared on a 50 μmol scale. The yield of the product was 3.2 mg, and its estimated purity by LCMS analysis was 100%. Analysis condition A: Retention time=1.73 min; ESI-MS(+) m/z [M+2H]²⁺: 1210.1.

### Preparation of Example 1125

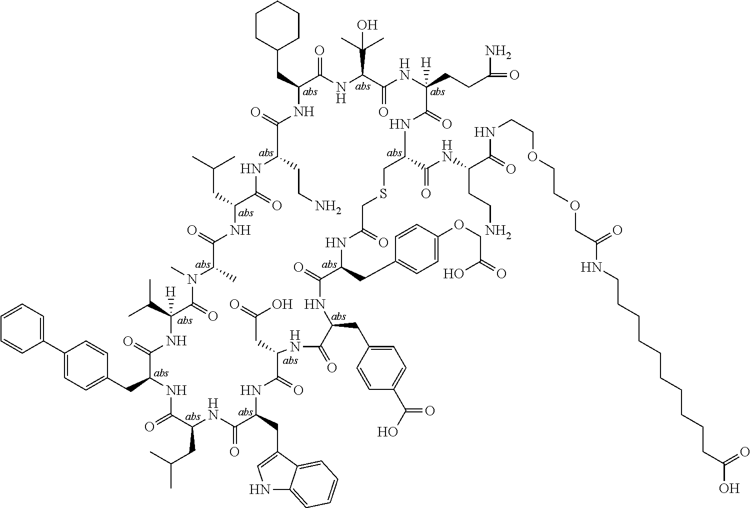 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1125 was prepared on a 50 μmol scale. The yield of the product was 35 mg, and its estimated purity by LCMS analysis was 100%. Analysis condition A: Retention time=1.82 min; ESI-MS(+) m/z [M+2H]²⁺: 1217.3.

### Preparation of Example 1126

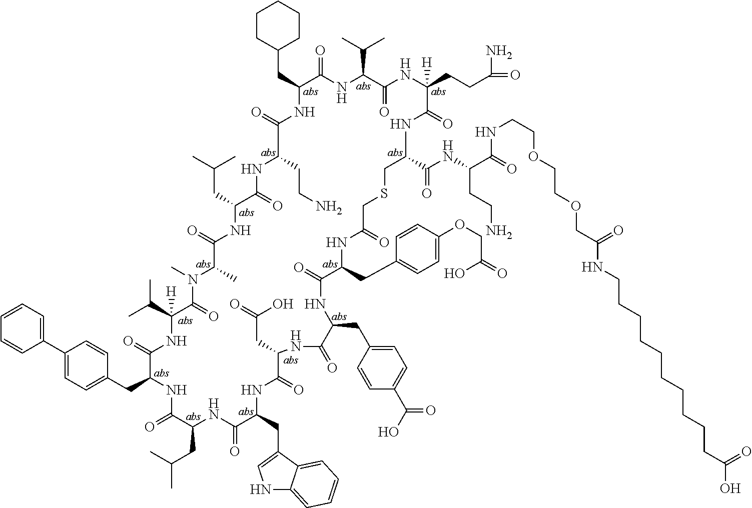 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1126 was prepared on a 50 μmol scale. The yield of the product was 20.9 mg, and its estimated purity by LCMS analysis was 97.1%. Analysis condition B: Retention time=1.94 min; ESI-MS(+) m/z [M+2H]²⁺: 1210.

### Preparation of Example 1127

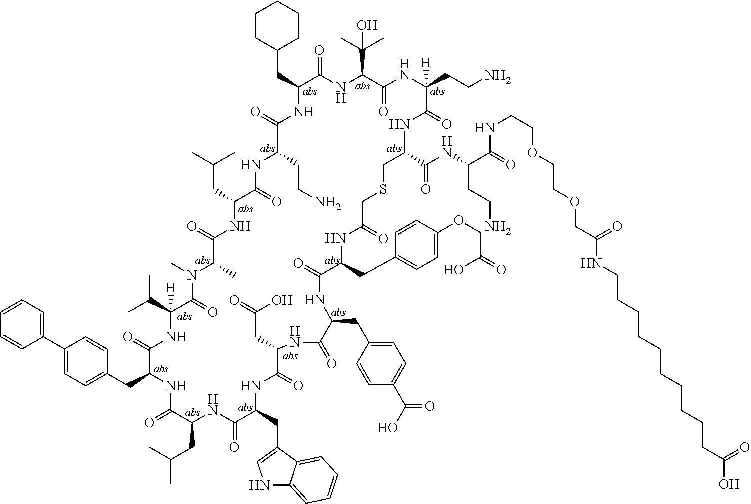 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCN)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1127 was prepared on a 50 μmol scale. The yield of the product was 12.7 mg, and its estimated purity by LCMS analysis was 100%. Analysis condition A: Retention time=1.76 min; ESI-MS(+) m/z [M+3H]³⁺: 803.1.

### Preparation of Example 1128

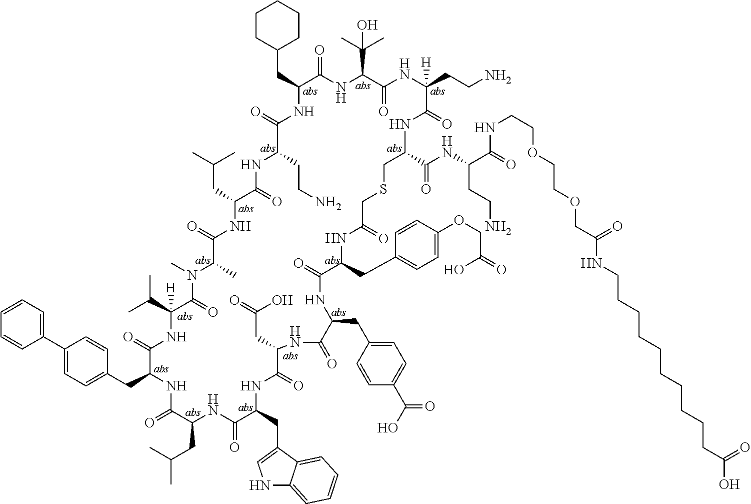 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCN)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1128 was prepared on a 50 μmol scale. The yield of the product was 7.6 mg, and its estimated purity by LCMS analysis was 100%. Analysis condition A: Retention time=1.75 min; ESI-MS(+) m/z [M+3H]³⁺: 797.

### Preparation of Example 1129

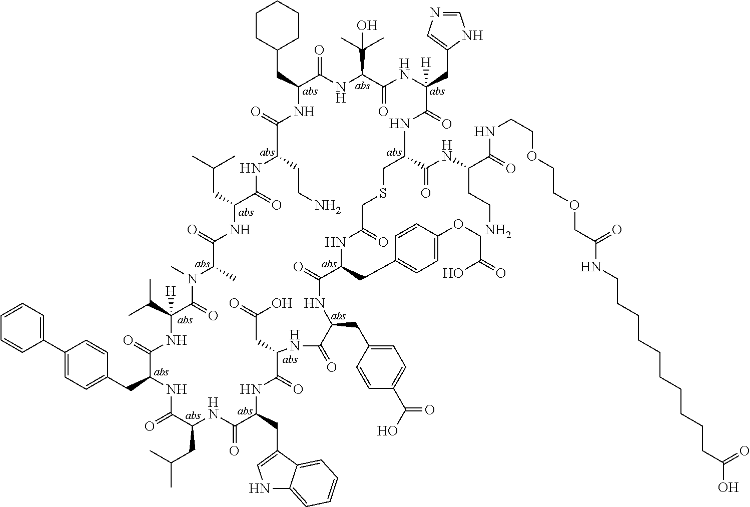 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1129 was prepared on a 50 μmol scale. The yield of the product was 11 mg, and its estimated purity by LCMS analysis was 90%. Analysis condition B: Retention time=1.87 min; ESI-MS(+) m/z [M+2H]²⁺: 1222.2.

### Preparation of Example 1130

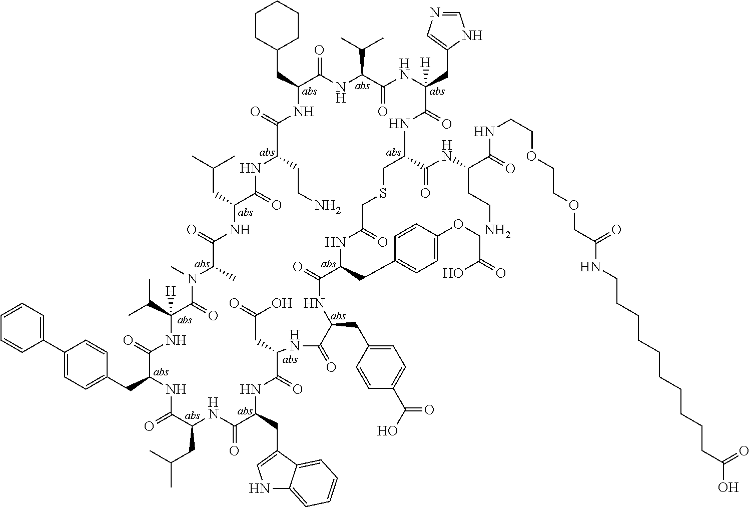 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](Cc2cnc[nH]2)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1130 was prepared on a 50 μmol scale. The yield of the product was 10.4 mg, and its estimated purity by LCMS analysis was 97.2%. Analysis condition B: Retention time=1.86 min; ESI-MS(+) m/z [M+2H]²⁺: 1214.2.

### Preparation of Example 1131

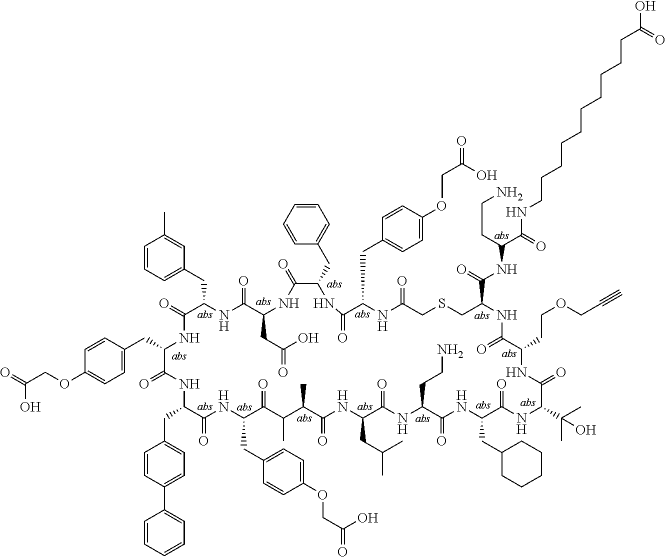

Example 1131 was prepared on a 50 μmol scale. The yield of the product was 36.2 mg, and its estimated purity by LCMS analysis was 89.5%. Analysis condition A: Retention time=1.58 min; ESI-MS(+) m/z [M+3H]³⁺: 821.2.

### Preparation of Example 1132

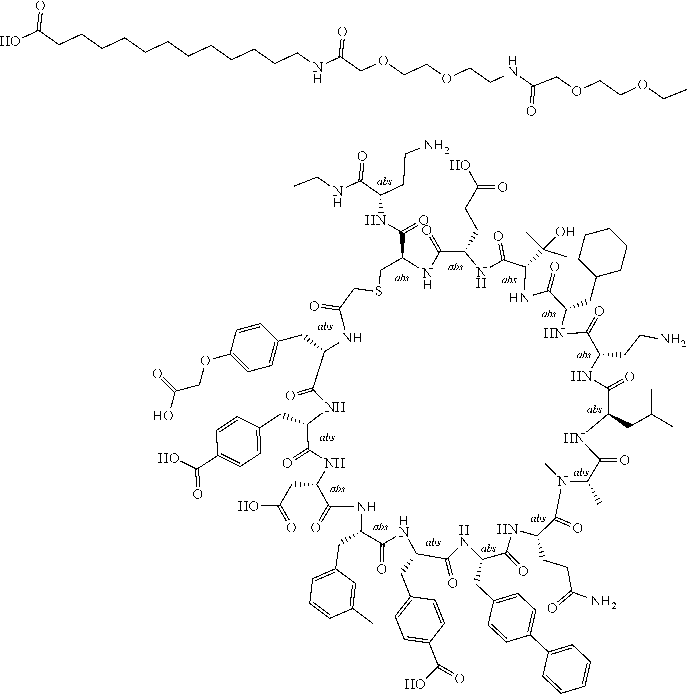 [SMILES: CCOCCOCC(=O)NCCOCCOCC(=O)NCCCCCCCCCCCCC(=O)O ; CCNC(=O)[C@H](CCN)NC(=O)[C@@H]1CSCC(=O)N[C@@H](Cc2ccc(OCC(=O)O)cc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](CCC(N)=O)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCC(=O)O)C(=O)N1]

Example 1132 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 57.4 mg, and its estimated purity by LCMS analysis was 92.9%. Analysis condition: A Retention time=1.45 min; ESI-MS(+) m/z [M+2H]²⁺: 1345.5.

### Preparation of Example 1133

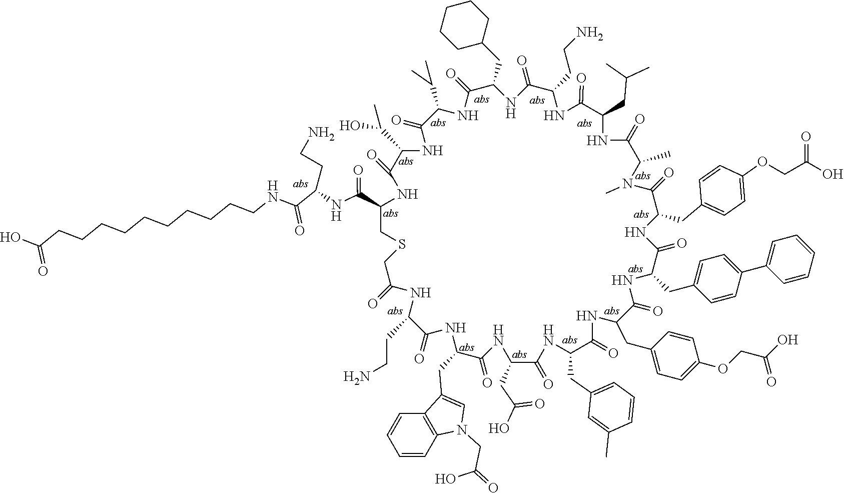 [SMILES: Cc1cccc(C[C@@H]2NC(=O)[C@H](CC(=O)O)NC(=O)[C@H](Cc3cn(CC(=O)O)c4ccccc34)NC(=O)[C@H](CCN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)C(Cc3ccc(OCC(=O)O)cc3)NC2=O)c1]

Example 1133 was prepared on a 30 μmol scale. The yield of the product was 13.9 mg, and its estimated purity by LCMS analysis was 93.10%. Analysis condition B: Retention time=1.84 min; ESI-MS(+) m/z [M+2H]²⁺: 1192.1.

### Preparation of Example 1134

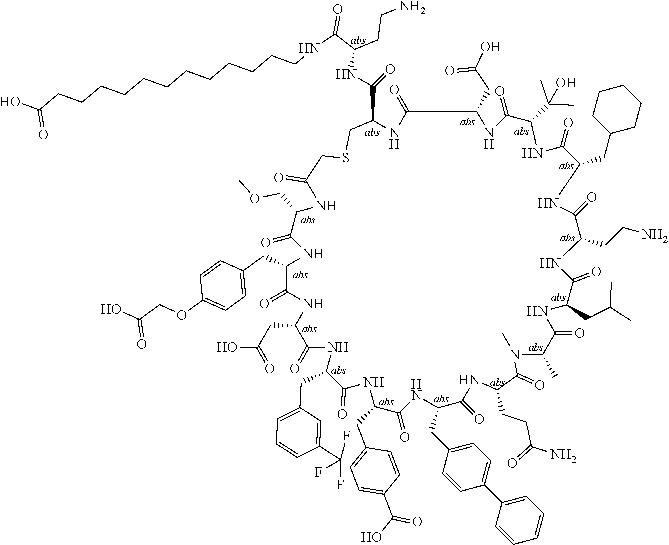 [SMILES: COC[C@@H]1NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1134 was prepared on a 50 μmol scale. The yield of the product was 19.2 mg, and its estimated purity by LCMS analysis was 95.6%. Analysis condition A: Retention time=1.79 min; ESI-MS(+) m/z [M+2H]²⁺: 1175.1.

### Preparation of Example 1135

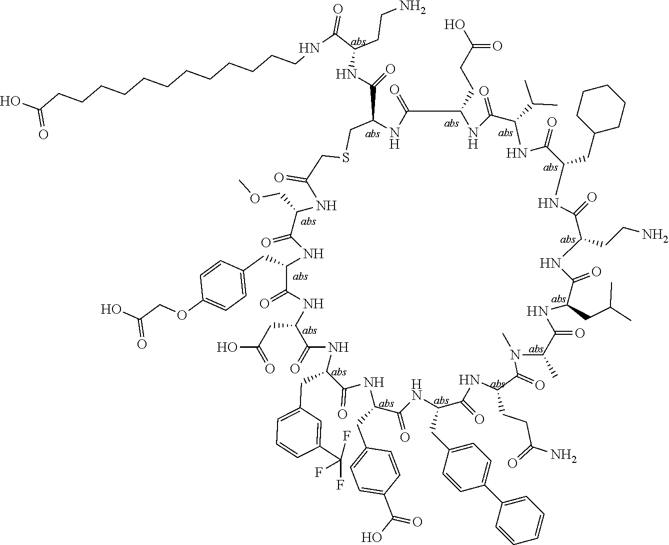 [SMILES: COC[C@@H]1NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1135 was prepared on a 50 μmol scale. The yield of the product was 16.4 mg, and its estimated purity by LCMS analysis was 100%. Analysis condition A: Retention time=1.95 min; ESI-MS(+) m/z [M+2H]²⁺: 1174.3.

### Preparation of Example 1136

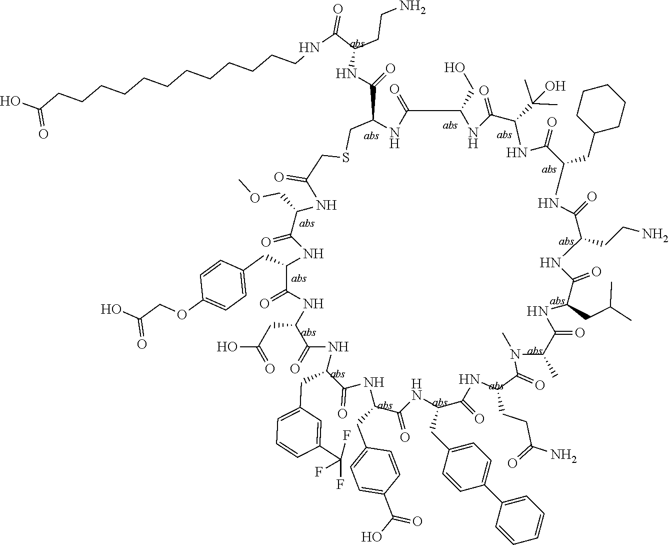 [SMILES: COC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1136 was prepared on a 50 μmol scale. The yield of the product was 25.5 mg, and its estimated purity by LCMS analysis was 92.6%. Analysis condition A: Retention time=1.71 min; ESI-MS(+) m/z [M+2H]²⁺: 1161.1.

### Preparation of Example 1137

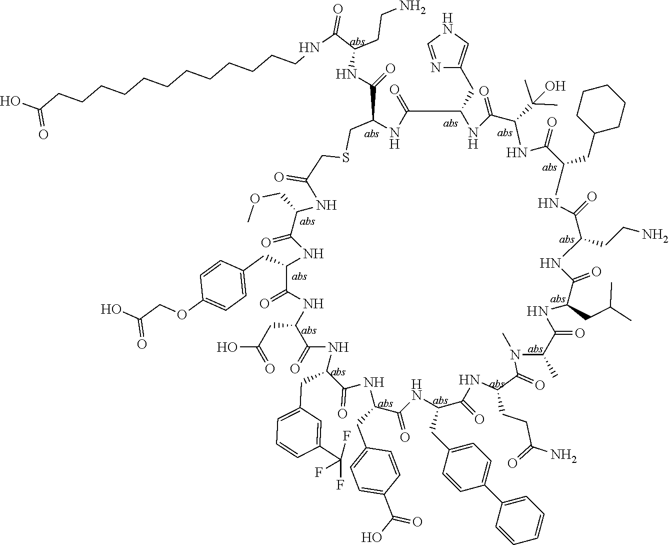 [SMILES: COC[C@@H]1NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1137 was prepared on a 50 μmol scale. The yield of the product was 55 mg, and its estimated purity by LCMS analysis was 93.4%. Analysis condition A: Retention time=1.8 min; ESI-MS(+) m/z [M+3H]³⁺: 791.3.

### Preparation of Example 1138

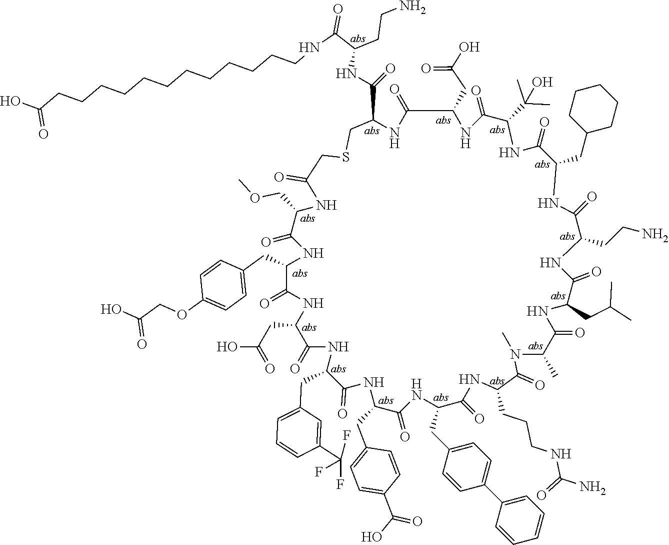 [SMILES: COC[C@@H]1NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1138 was prepared on a 50 μmol scale. The yield of the product was 35.2 mg, and its estimated purity by LCMS analysis was 92.4%. Analysis condition A: Retention time=1.76 min; ESI-MS(+) m/z [M+2H]²⁺: 1189.9.

### Preparation of Example 1139

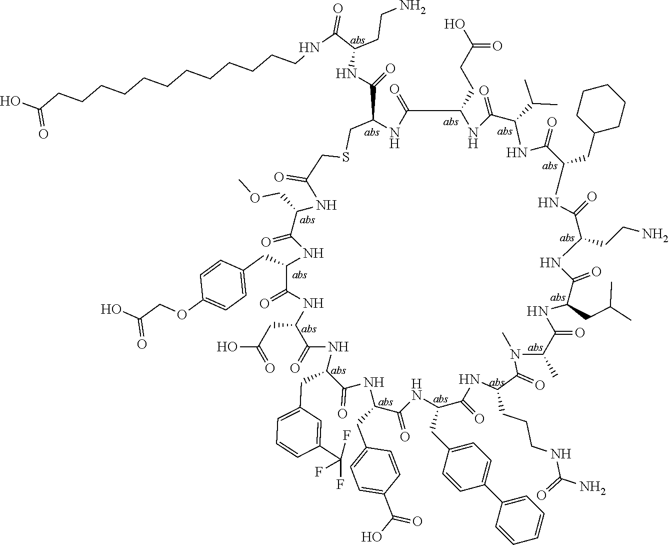 [SMILES: COC[C@@H]1NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1139 was prepared on a 50 μmol scale. The yield of the product was 18.4 mg, and its estimated purity by LCMS analysis was 91.8%. Analysis condition A: Retention time=1.63 min; ESI-MS(+) m/z [M+2H]²⁺: 1189.

### Preparation of Example 1140

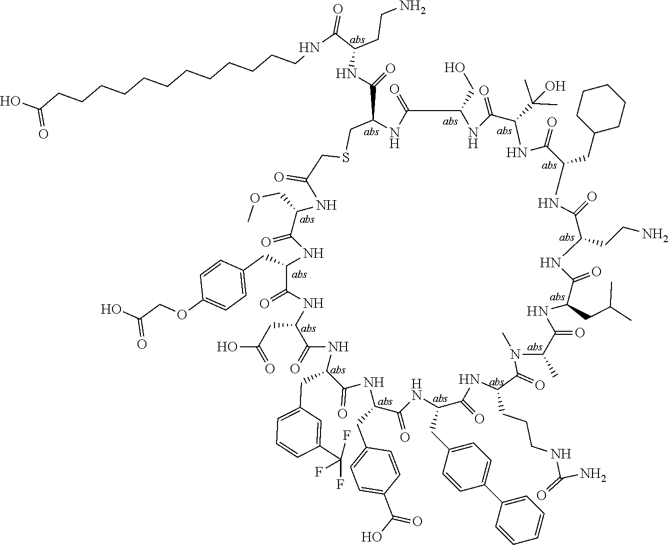 [SMILES: COC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1140 was prepared on a 50 μmol scale. The yield of the product was 27.4 mg, and its estimated purity by LCMS analysis was 90.1%. Analysis condition A: Retention time=1.86 min; ESI-MS(+) m/z [M+2H]²⁺: 1176.2.

### Preparation of Example 1141

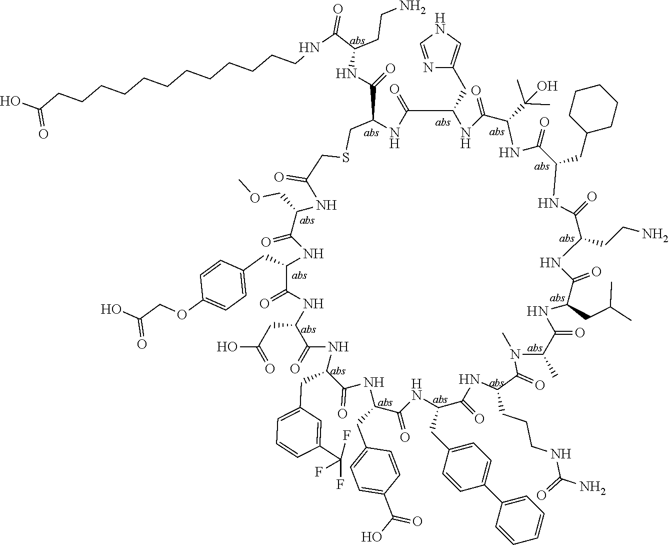 [SMILES: COC[C@@H]1NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1141 was prepared on a 50 μmol scale. The yield of the product was 25.3 mg, and its estimated purity by LCMS analysis was 95.1%. Analysis condition A: Retention time=1.74 min; ESI-MS(+) m/z [M+2H]²⁺: 1201.2.

### Preparation of Example 1142

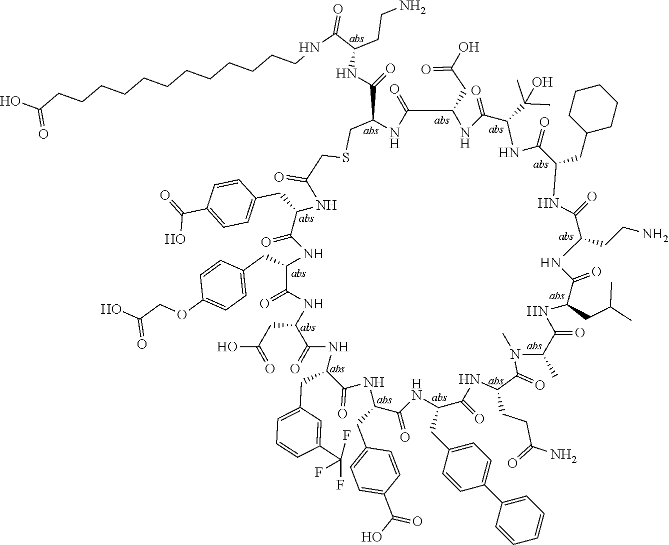 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1142 was prepared on a 50 μmol scale. The yield of the product was 60.1 mg, and its estimated purity by LCMS analysis was 92.2%. Analysis condition A: Retention time=1.66 min; ESI-MS(+) m/z [M+2H]²⁺: 1220.4.

### Preparation of Example 1143

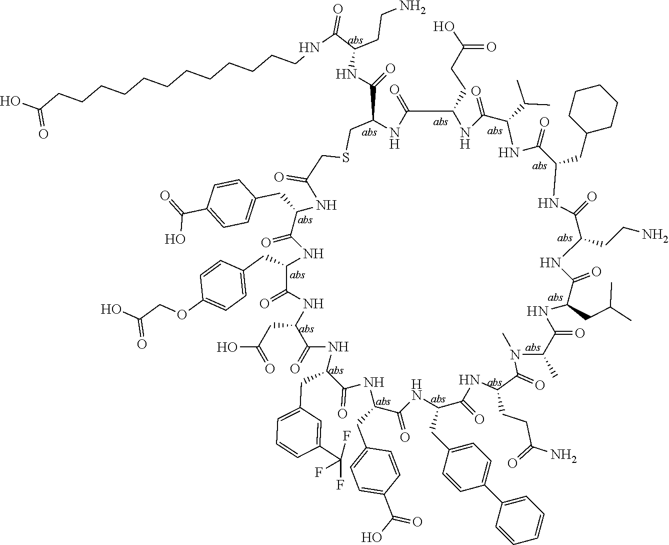 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1143 was prepared on a 50 μmol scale. The yield of the product was 22 mg, and its estimated purity by LCMS analysis was 91.7%. Analysis condition A: Retention time=1.73 min; ESI-MS(+) m/z [M+2H]²⁺: 1219.2.

### Preparation of Example 1144

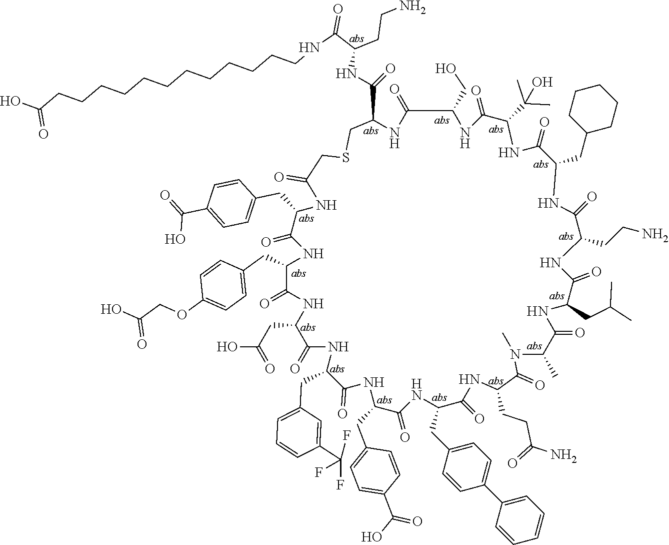 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 1144 was prepared on a 50 μmol scale. The yield of the product was 15.5 mg, and its estimated purity by LCMS analysis was 97.40%. Analysis condition B: Retention time=1.98 min; ESI-MS(+) m/z [M+2H]²⁺: 1206.9.

### Preparation of Example 1145

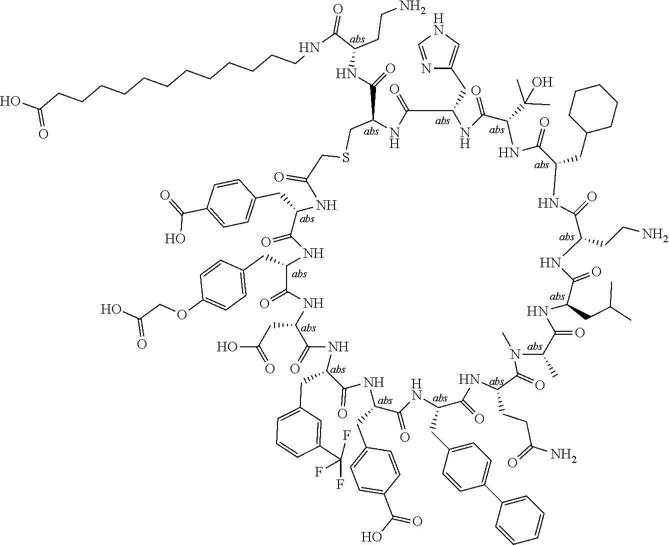 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1145 was prepared on a 50 μmol scale. The yield of the product was 48.8 mg, and its estimated purity by LCMS analysis was 91.4%. Analysis condition A: Retention time=1.56 min; ESI-MS(+) m/z [M+2H]²⁺: 1231.1.

### Preparation of Example 1146

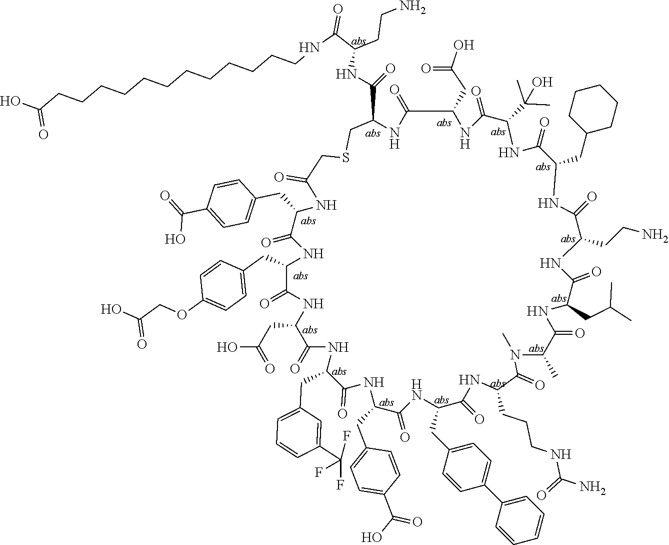 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1146 was prepared on a 50 μmol scale. The yield of the product was 20.1 mg, and its estimated purity by LCMS analysis was 100%. Analysis condition A: Retention time=1.61 min; ESI-MS(+) m/z [M+2H]²⁺: 1235.2.

### Preparation of Example 1147

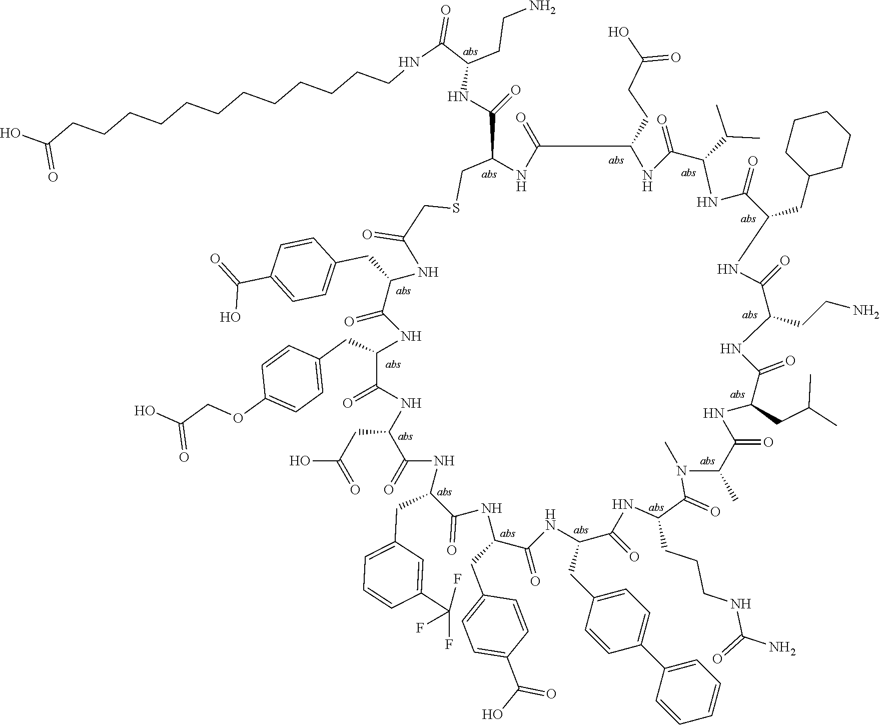 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1147 was prepared on a 50 μmol scale. The yield of the product was 15.3 mg, and its estimated purity by LCMS analysis was 93.2%. Analysis condition A: Retention time=1.51 min; ESI-MS(+) m/z [M+2H]²⁺: 1234.1.

### Preparation of Example 1148

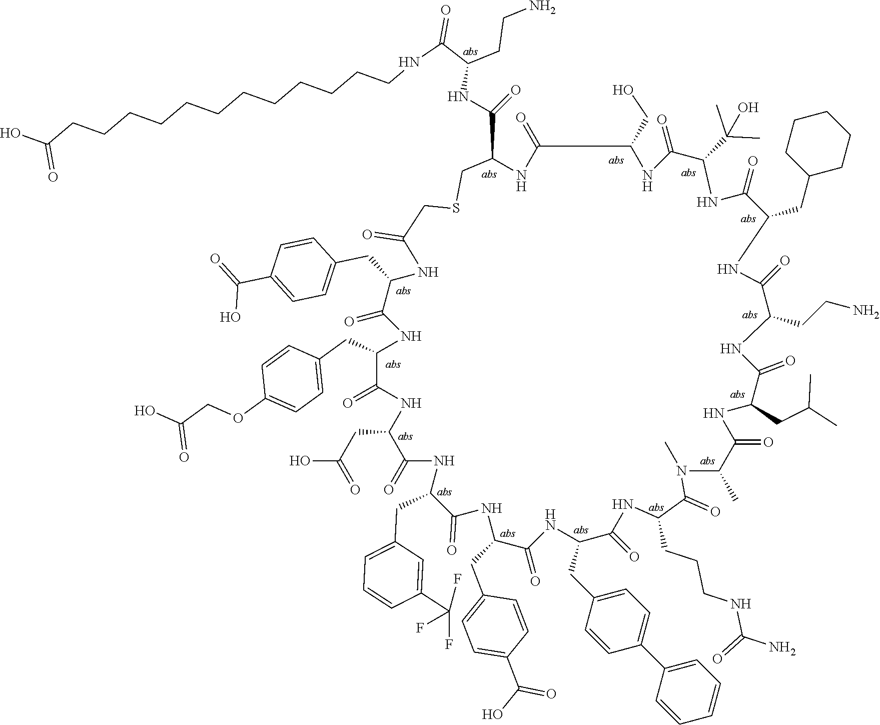 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 1148 was prepared on a 50 μmol scale. The yield of the product was 27 mg, and its estimated purity by LCMS analysis was 95.4%. Analysis condition A: Retention time=1.75 min; ESI-MS(+) m/z [M+2H]²⁺: 1220.9.

### Preparation of Example 1149

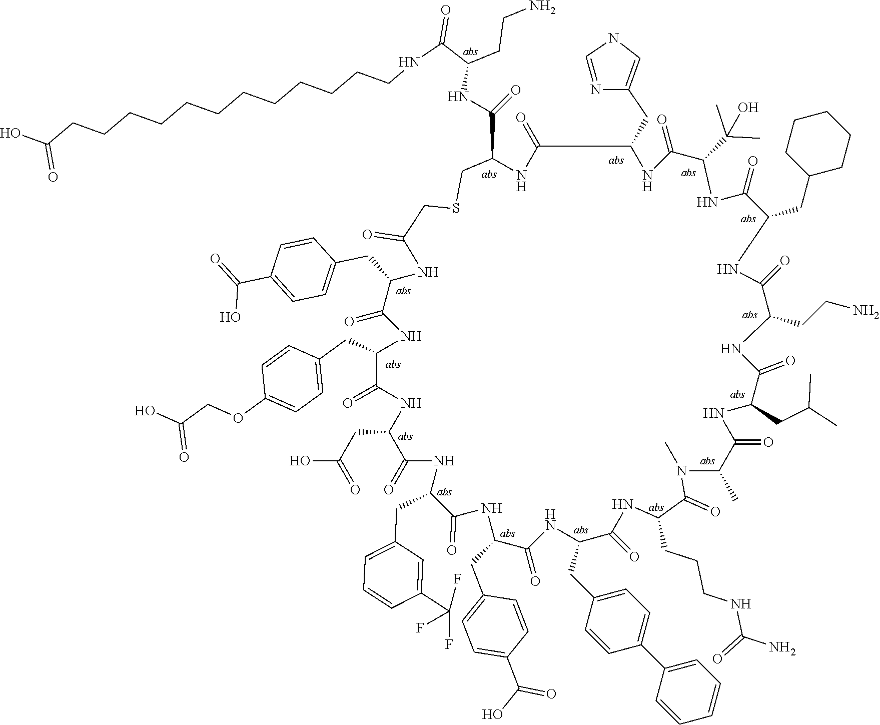 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1149 was prepared on a 50 μmol scale. The yield of the product was 46.8 mg, and its estimated purity by LCMS analysis was 93.9%. Analysis condition A: Retention time=1.74 min; ESI-MS(+) m/z [M+2H]²⁺: 1245.9.

### Preparation of Example 1150

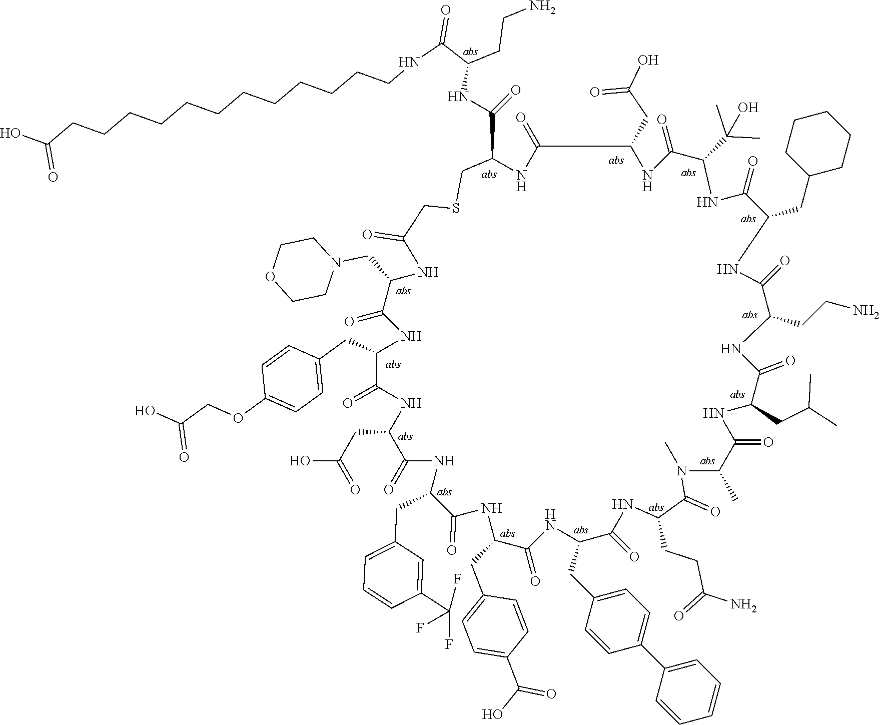 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1150 was prepared on a 50 μmol scale. The yield of the product was 23.8 mg, and its estimated purity by LCMS analysis was 92.1%. Analysis condition A: Retention time=1.6 min; ESI-MS(+) m/z [M+2H]²⁺: 1203.1.

### Preparation of Example 1151

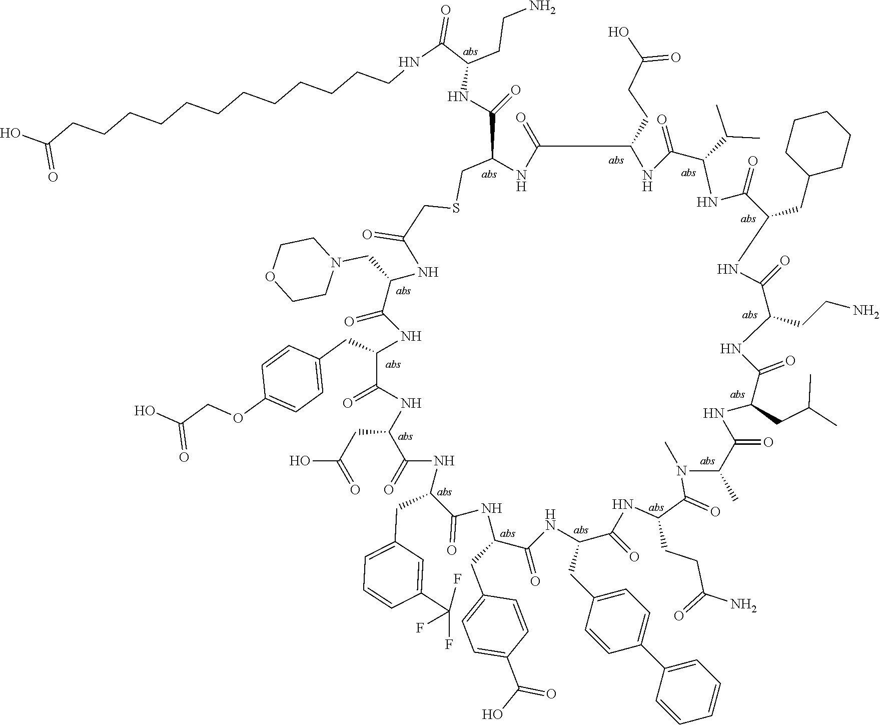 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1151 was prepared on a 50 μmol scale. The yield of the product was 10.6 mg, and its estimated purity by LCMS analysis was 92.3%. Analysis condition B: Retention time=1.86 min; ESI-MS(+) m/z [M+2H]²⁺: 1202.3.

### Preparation of Example 1152

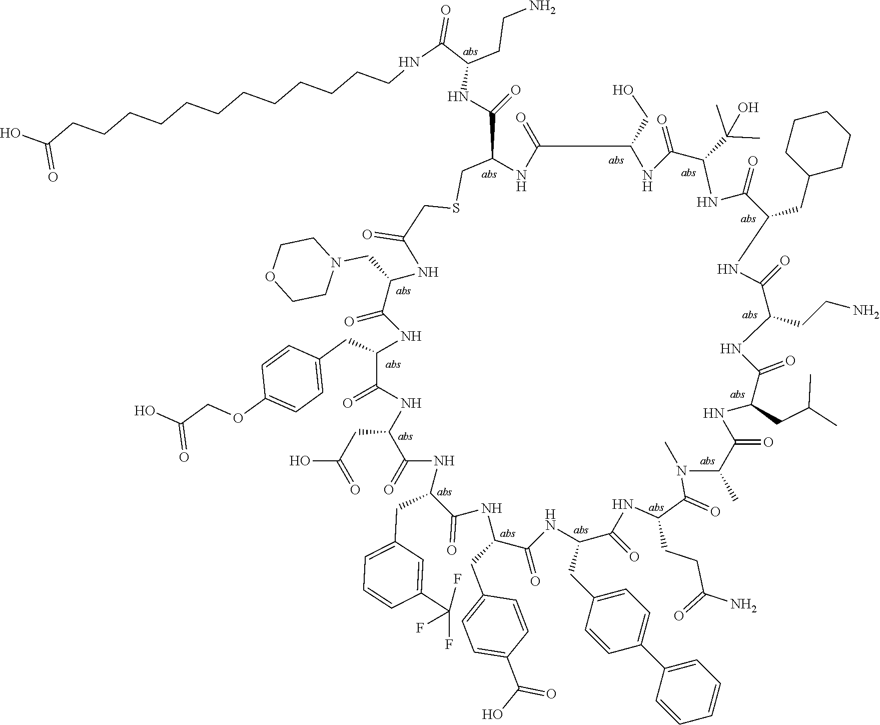 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 1152 was prepared on a 50 μmol scale. The yield of the product was 3.1 mg, and its estimated purity by LCMS analysis was 100%. Analysis condition A: Retention time=1.73 min; ESI-MS(+) m/z [M+2H]²⁺: 1189.

### Preparation of Example 1153

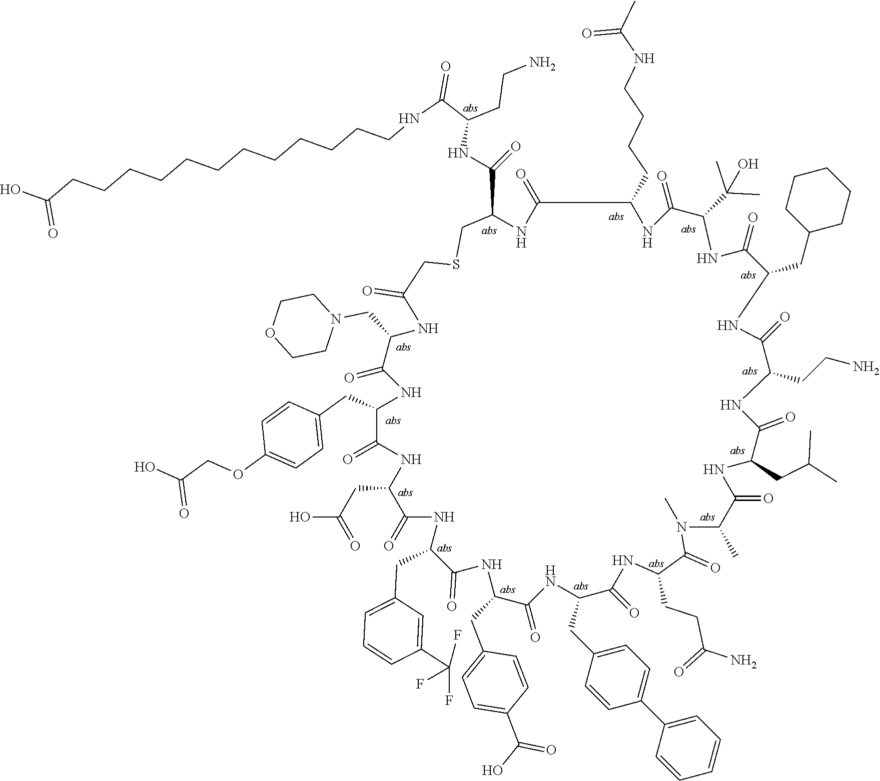 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC1=O]

Example 1153 was prepared on a 50 μmol scale. The yield of the product was 12.2 mg, and its estimated purity by LCMS analysis was 90.5%. Analysis condition B: Retention time=1.85 min; ESI-MS(+) m/z [M+2H]²⁺: 1231.1.

### Preparation of Example 1154

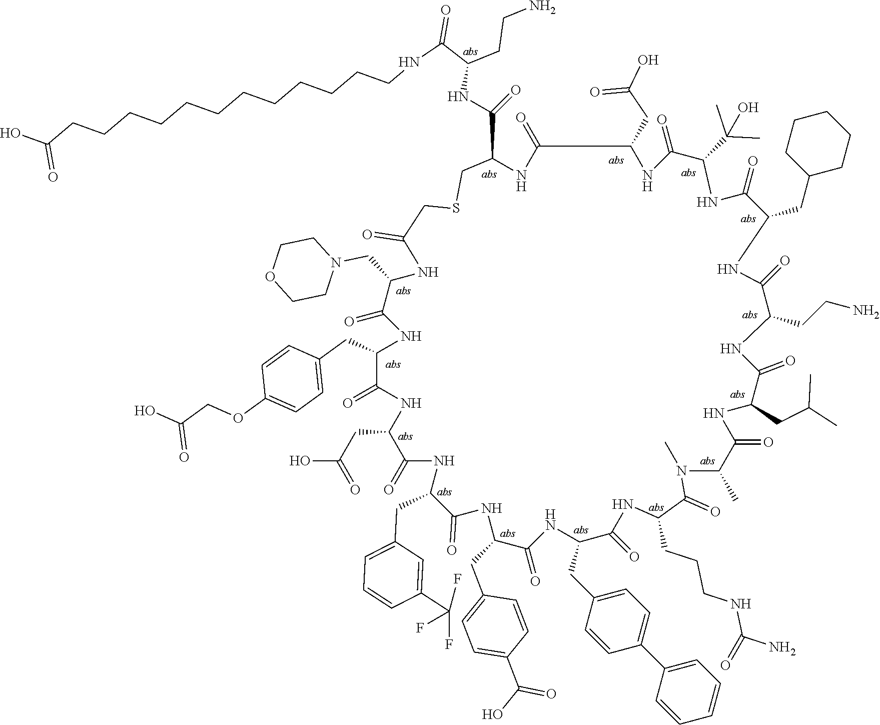 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1154 was prepared on a 50 μmol scale. The yield of the product was 18 mg, and its estimated purity by LCMS analysis was 96.3%. Analysis condition A: Retention time=1.81 min; ESI-MS(+) m/z [M+2H]²⁺: 1218.

### Preparation of Example 1155

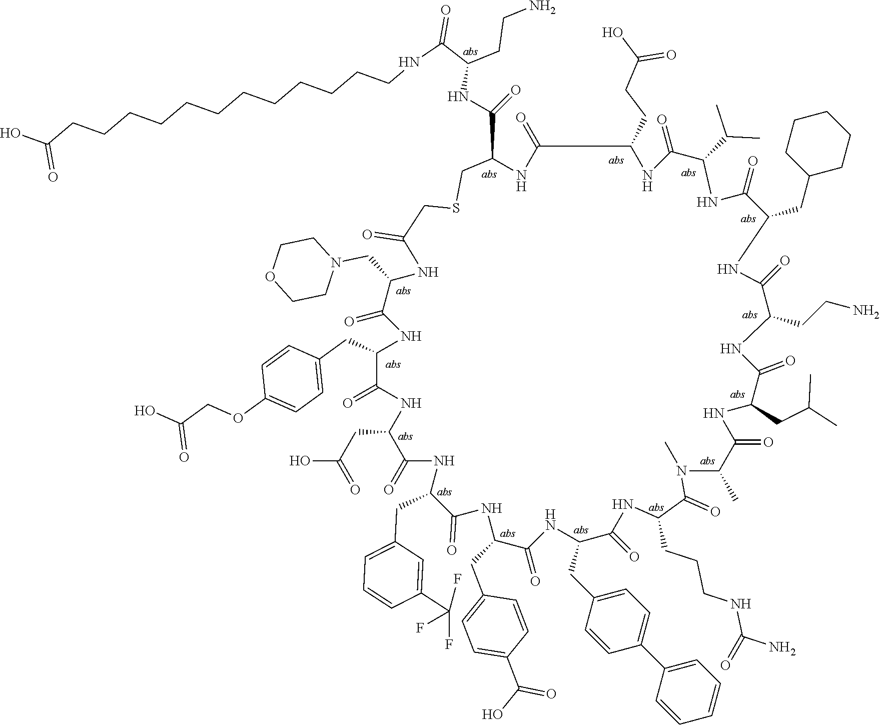 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1155 was prepared on a 50 μmol scale. The yield of the product was 11.2 mg, and its estimated purity by LCMS analysis was 90.2%. Analysis condition A: Retention time=1.62 min; ESI-MS(+) m/z [M+3H]³⁺: 811.3.

### Preparation of Example 1156

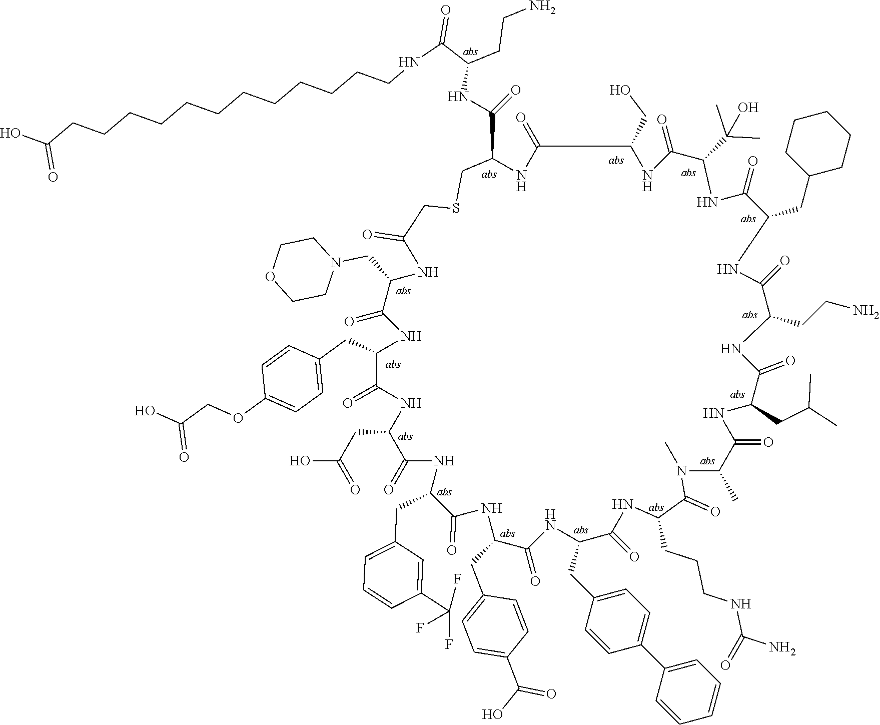 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 1156 was prepared on a 50 μmol scale. The yield of the product was 9.6 mg, and its estimated purity by LCMS analysis was 95.7%. Analysis condition A: Retention time=1.86 min; ESI-MS(+) m/z [M+3H]²⁺: 803.1.

### Preparation of Example 1157

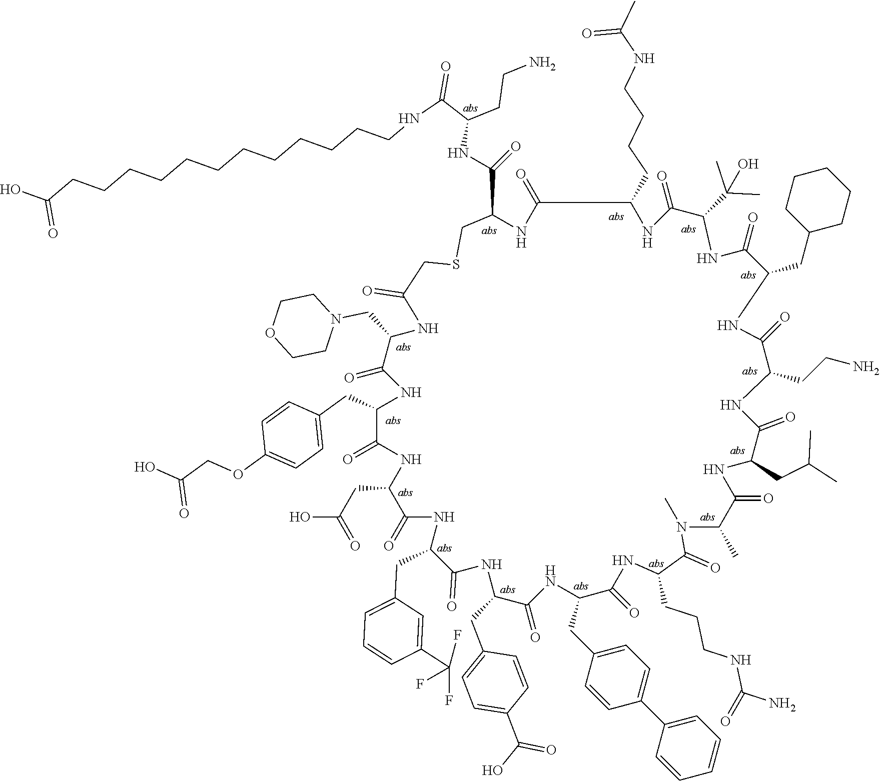 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC1=O]

Example 1157 was prepared on a 50 μmol scale. The yield of the product was 26.8 mg, and its estimated purity by LCMS analysis was 98.5%. Analysis condition A: Retention time=1.61 min; ESI-MS(+) m/z [M+2H]²⁺: 1245.2.

### Preparation of Example 1158

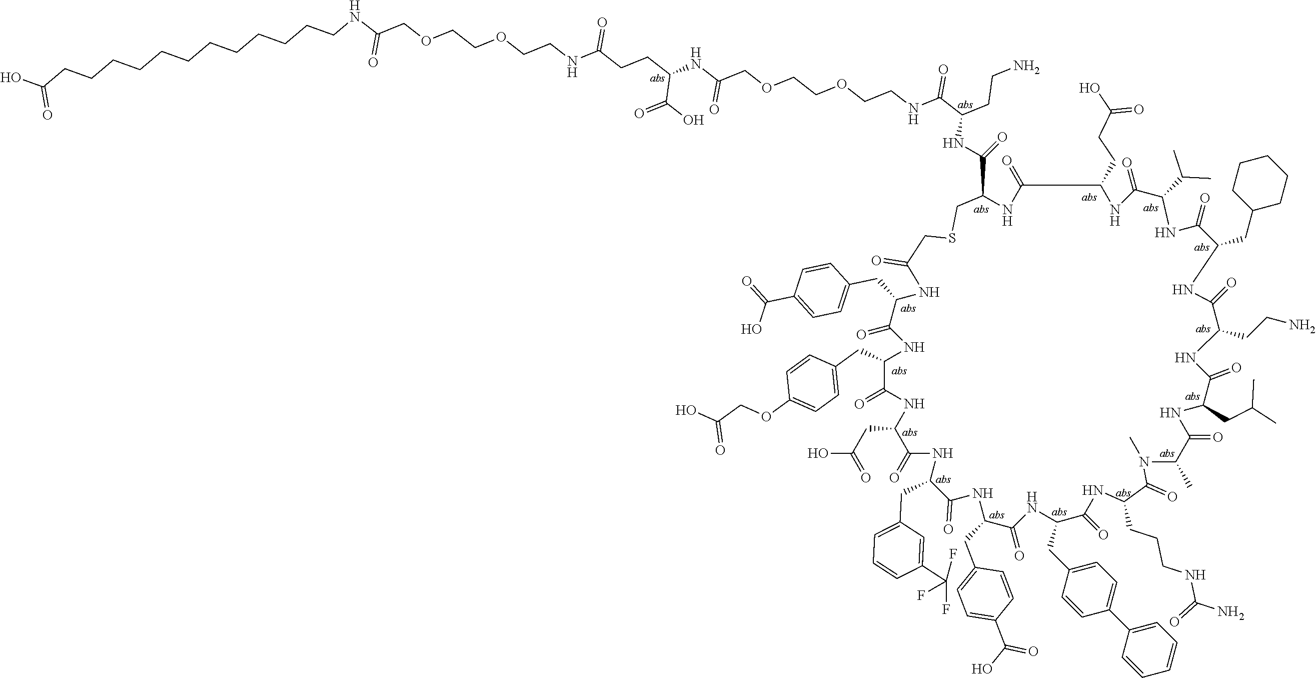 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1158 was prepared on a 50 μmol scale. The yield of the product was 13.2 mg, and its estimated purity by LCMS analysis was 94.3%. Analysis condition A: Retention time=1.53 min; ESI-MS(+) m/z [M+2H]²⁺: 1444.5.

### Preparation of Example 1159

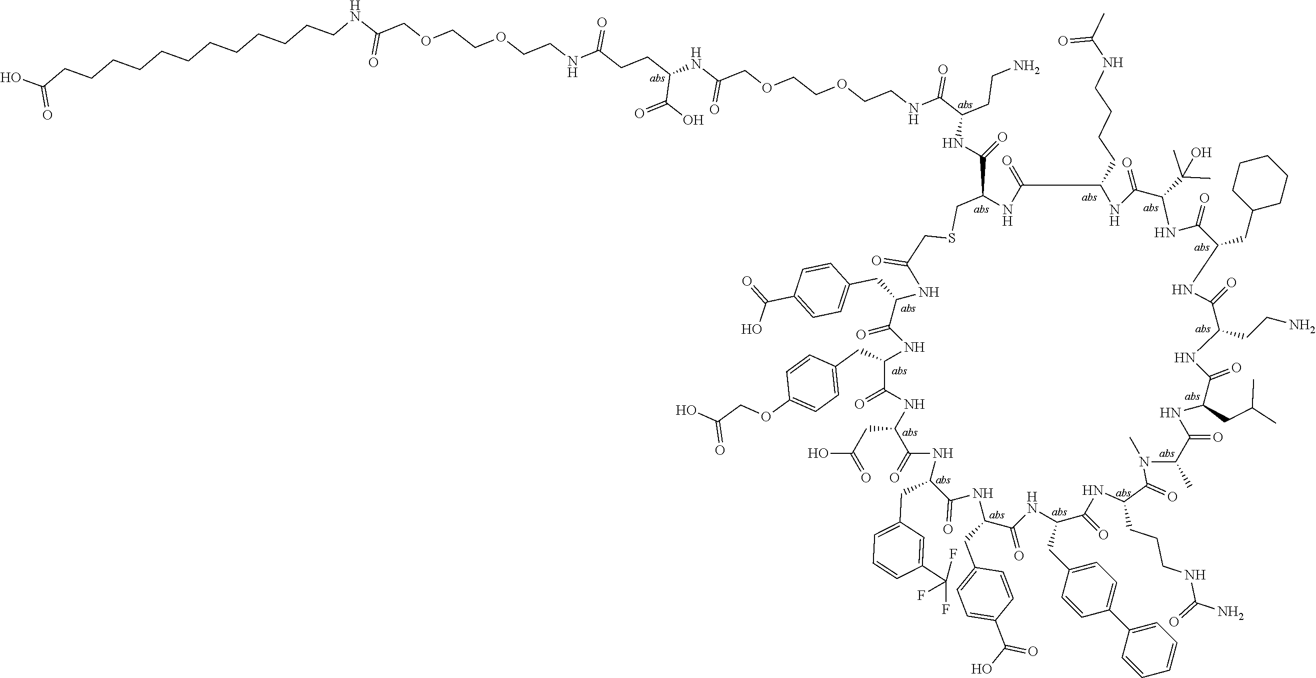 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 1159 was prepared on a 50 μmol scale. The yield of the product was 29.7 mg, and its estimated purity by LCMS analysis was 91.6%. Analysis condition A: Retention time=1.56 min; ESI-MS(+) m/z [M+2H]²⁺: 1472.

### Preparation of Example 1160

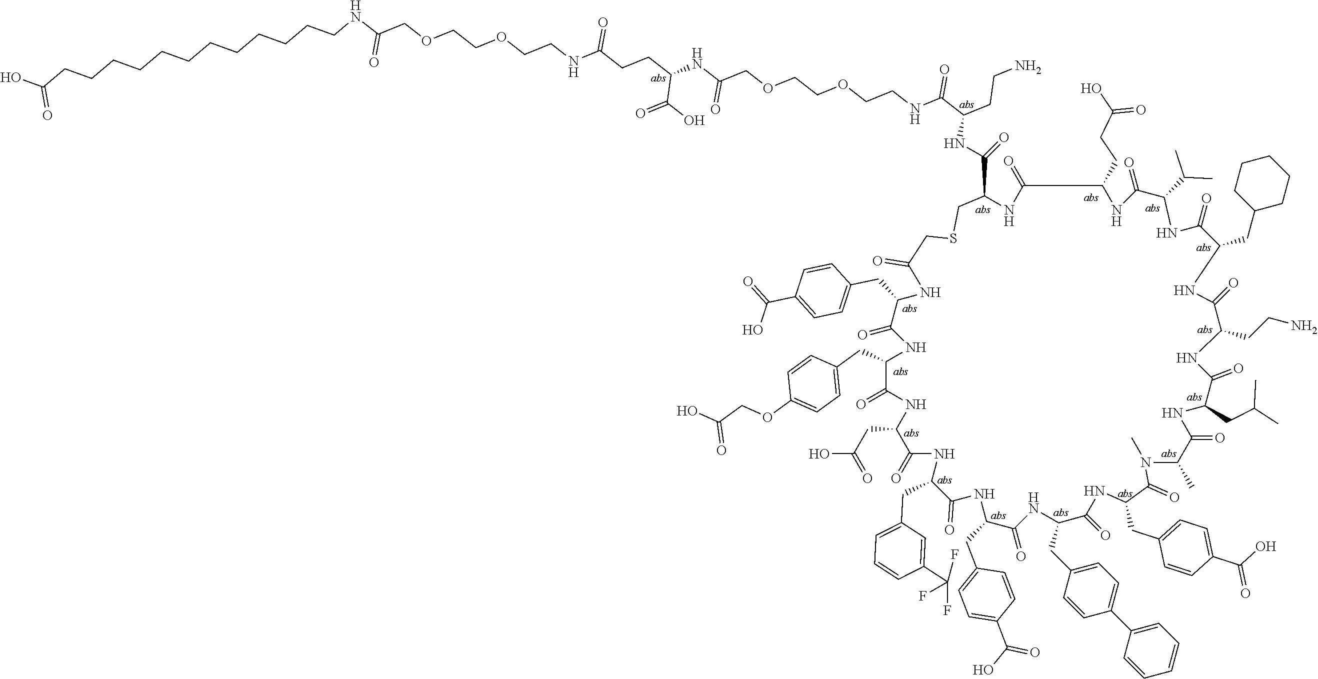 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1160 was prepared on a 50 μmol scale. The yield of the product was 17.1 mg, and its estimated purity by LCMS analysis was 100%. Analysis condition A: Retention time=1.99 min; ESI-MS(+) m/z [M+2H]²⁺: 1461.

### Preparation of Example 1161

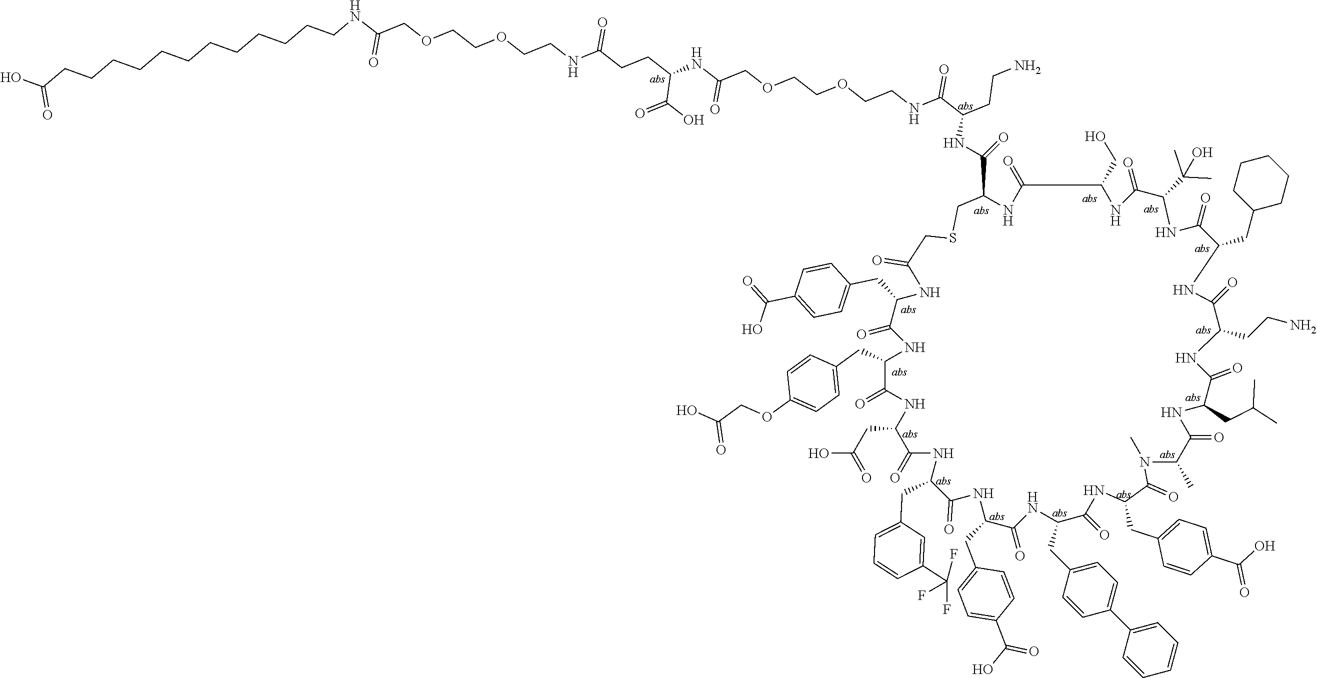 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1161 was prepared on a 50 μmol scale. The yield of the product was 24.8 mg, and its estimated purity by LCMS analysis was 100%. Analysis condition A: Retention time=2 min; ESI-MS(+) m/z [M+2H]²⁺: 1447.2.

### Preparation of Example 1162

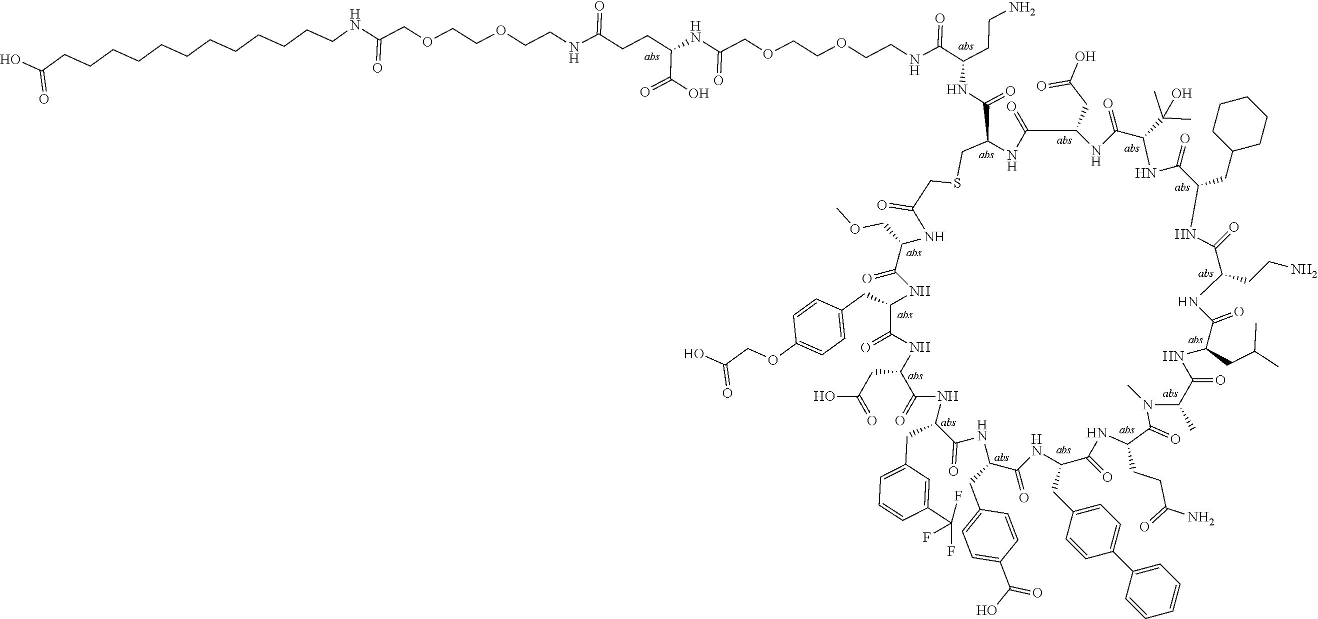 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1162 was prepared on a 50 μmol scale. The yield of the product was 19.7 mg, and its estimated purity by LCMS analysis was 100%. Analysis condition A: Retention time=1.6 min; ESI-MS(+) m/z [M+3H]³⁺: 924.5.

### Preparation of Example 1163

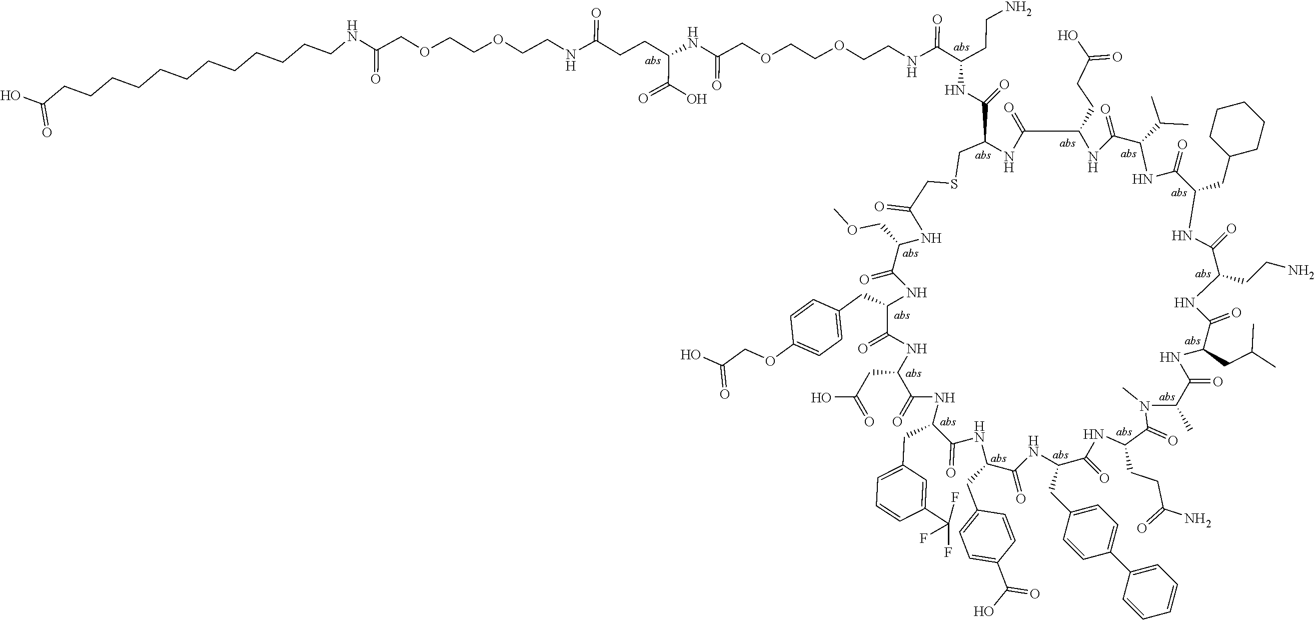

Example 1163 was prepared on a 50 μmol scale. The yield of the product was 7.8 mg, and its estimated purity by LCMS analysis was 98%. Analysis condition A: Retention time=1.64 min; ESI-MS(+) m/z [M+2H]²⁺: 1384.3.

### Preparation of Example 1164

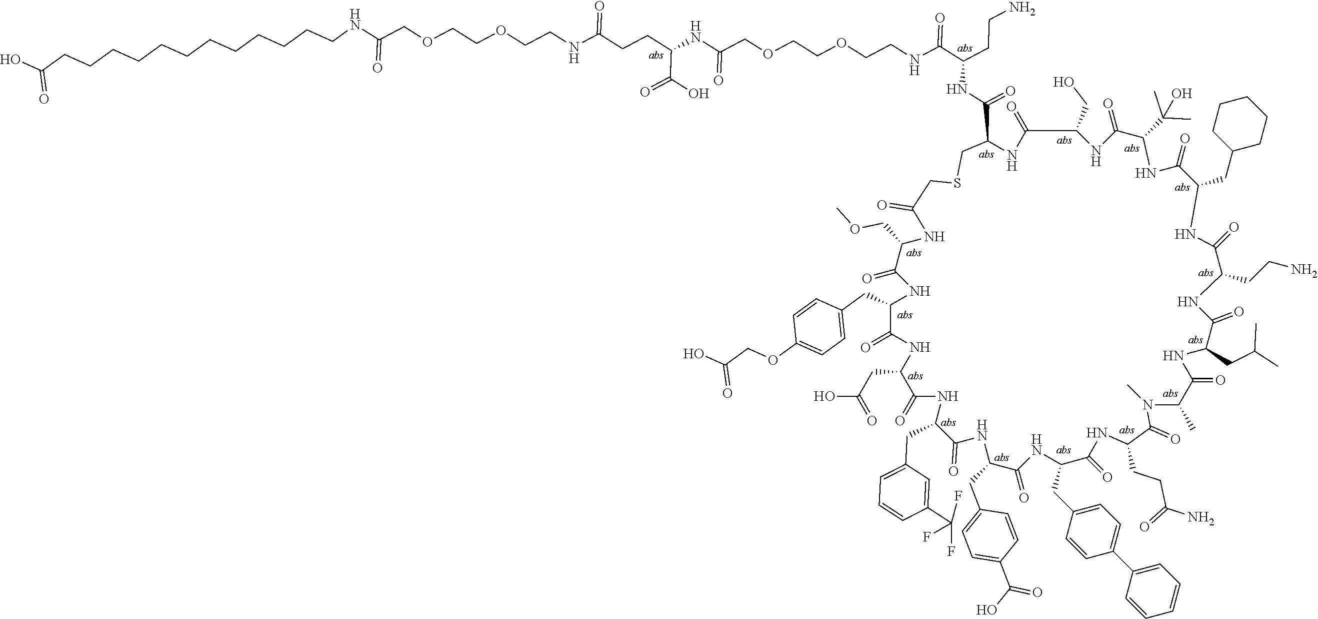 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1164 was prepared on a 50 μmol scale. The yield of the product was 10.7 mg, and its estimated purity by LCMS analysis was 95.3%. Analysis condition A: Retention time=1.74 min; ESI-MS (+) m/z [M+2H]²⁺: 1370.9.

### Preparation of Example 1165

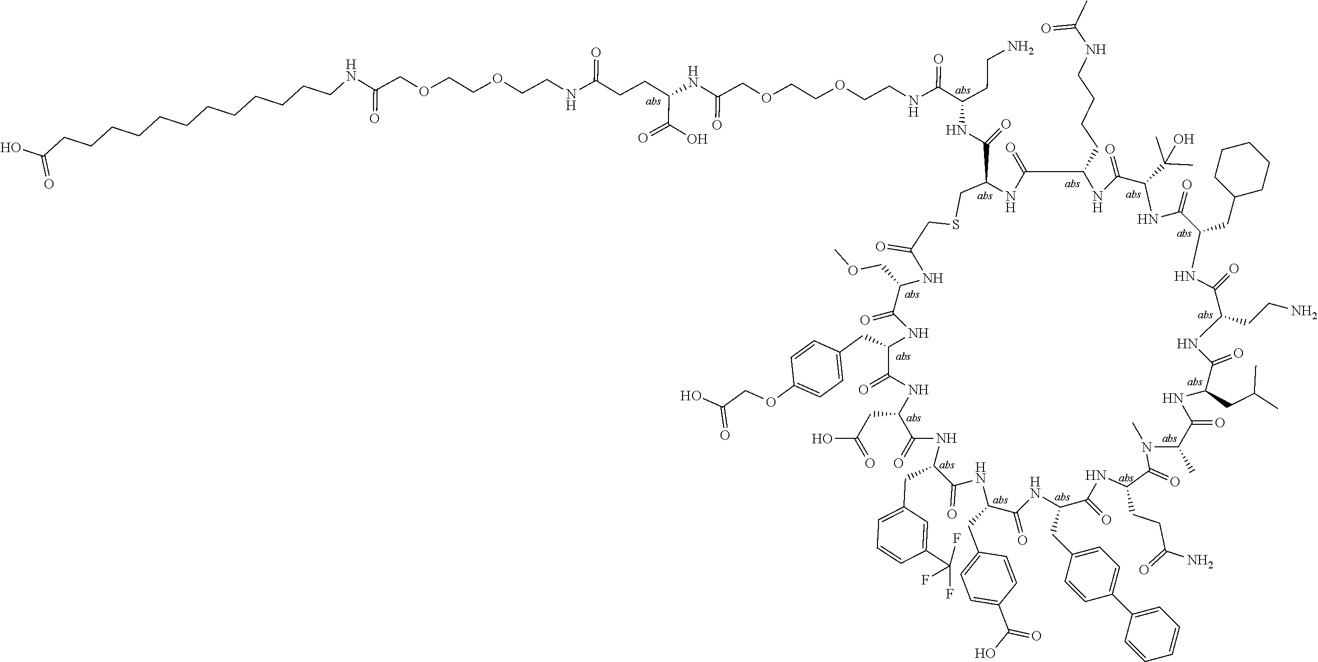 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCCCNC(C)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1165 was prepared on a 50 μmol scale. The yield of the product was 15.4 mg, and its estimated purity by LCMS analysis was 93.4%. Analysis condition A: Retention time=1.61 min; ESI-MS(+) m/z [M+2H]²⁺: 1413.

### Preparation of Example 1166

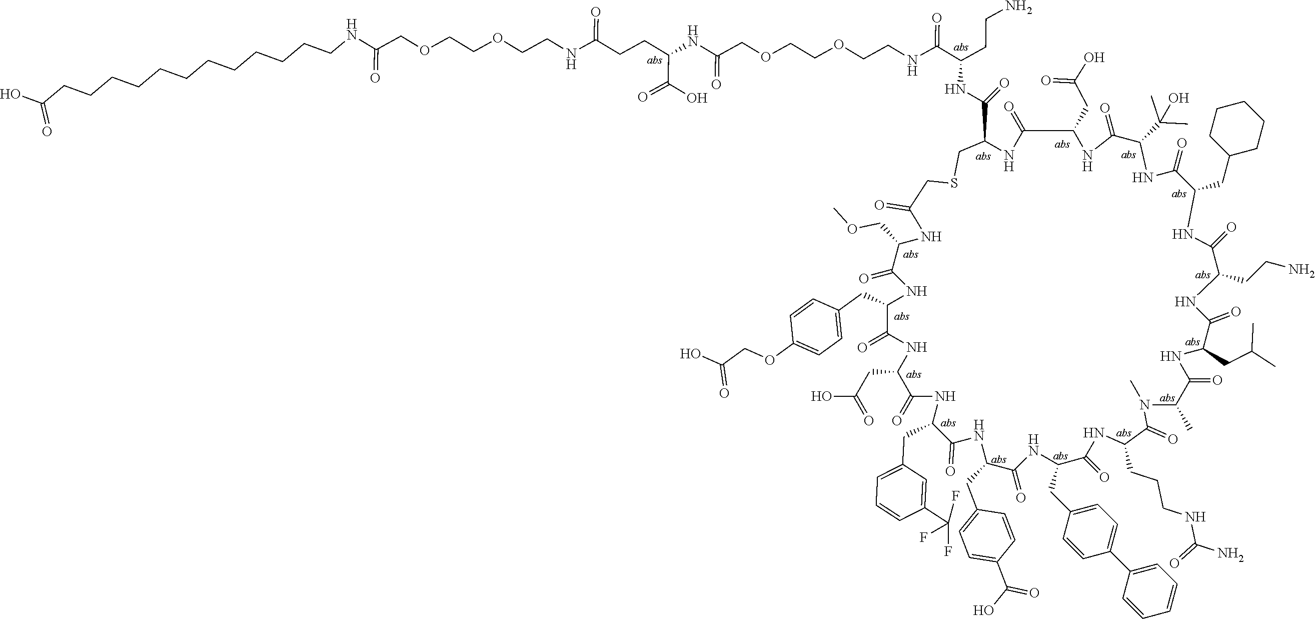 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1166 was prepared on a 50 μmol scale. The yield of the product was 16.7 mg, and its estimated purity by LCMS analysis was 960%. Analysis condition A: Retention time=1.99 min; ESI-MS(+) m/z [M+23H]³⁺: 933.9.

### Preparation of Example 1167

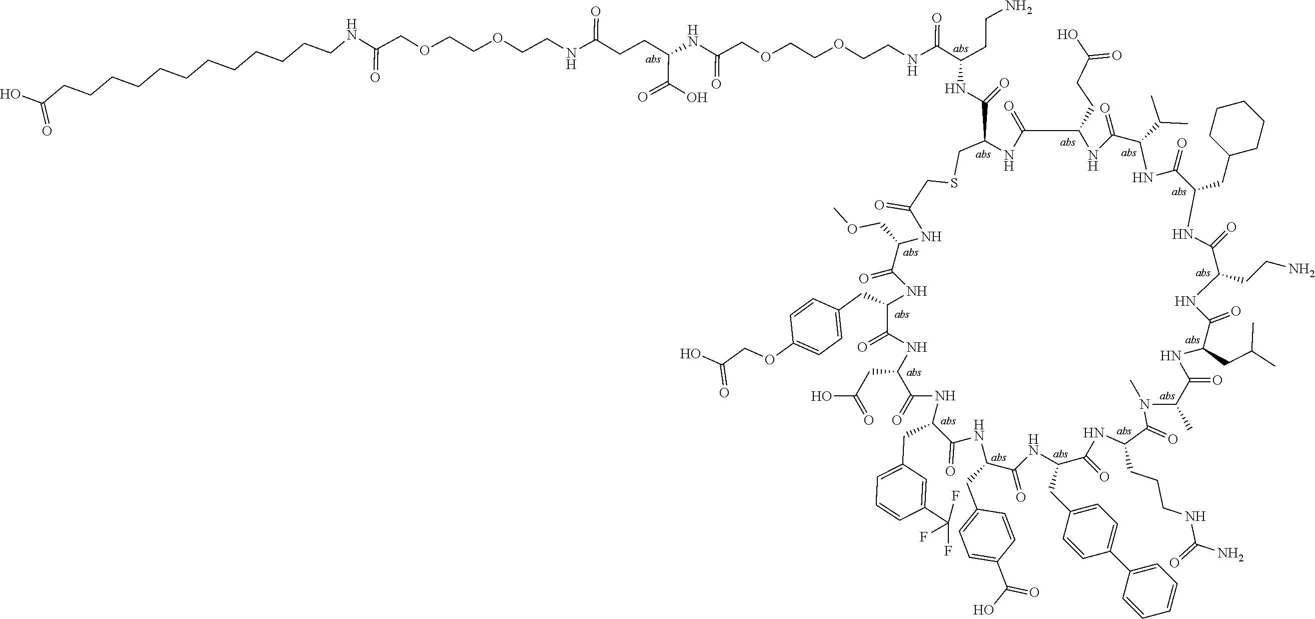 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1167 was prepared on a 50 μmol scale. The yield of the product was 10 mg, and its estimated purity by LCMS analysis was 92.9%. Analysis condition A: Retention time=1.6 min; ESI-MS(+) m/z [M+3H]³⁺: 933.2.

### Preparation of Example 1168

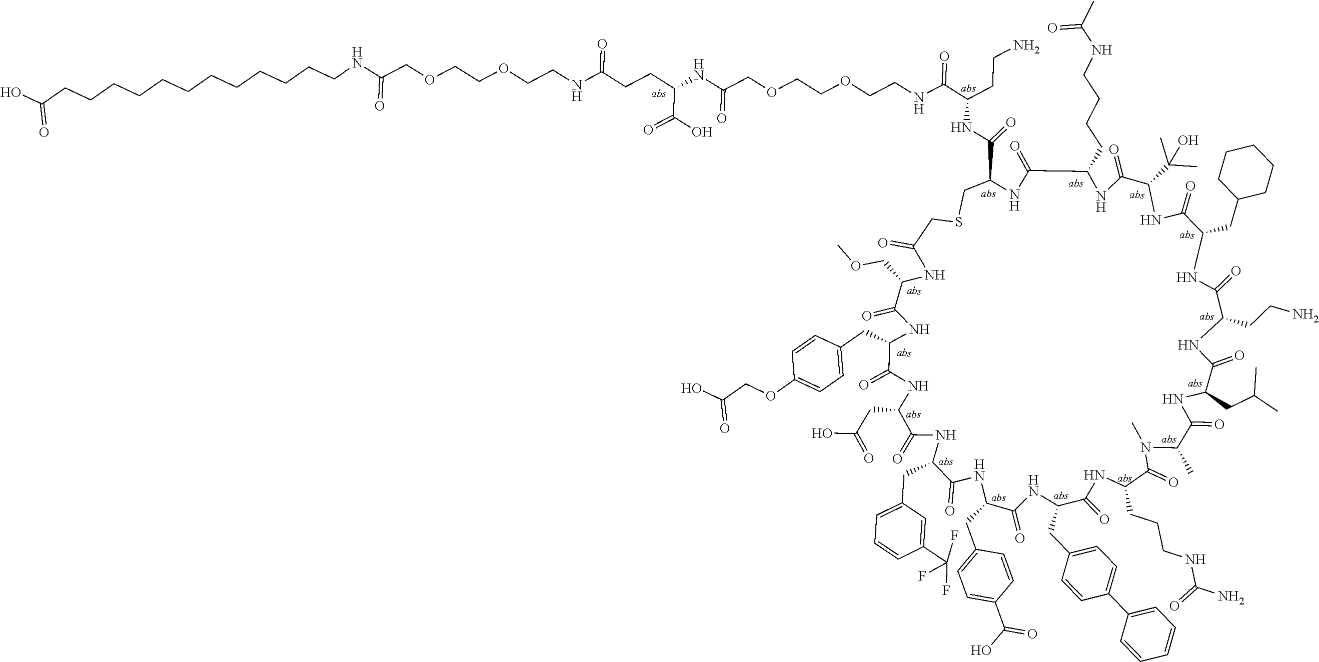

Example 1168 was prepared on a 50 μmol scale. The yield of the product was 25.1 mg, and its estimated purity by LCMS analysis was 100%. Analysis condition A: Retention time=1.62 min; ESI-MS(+) m/z [M+2H]²⁺: 1427.1.

### Preparation of Example 1169

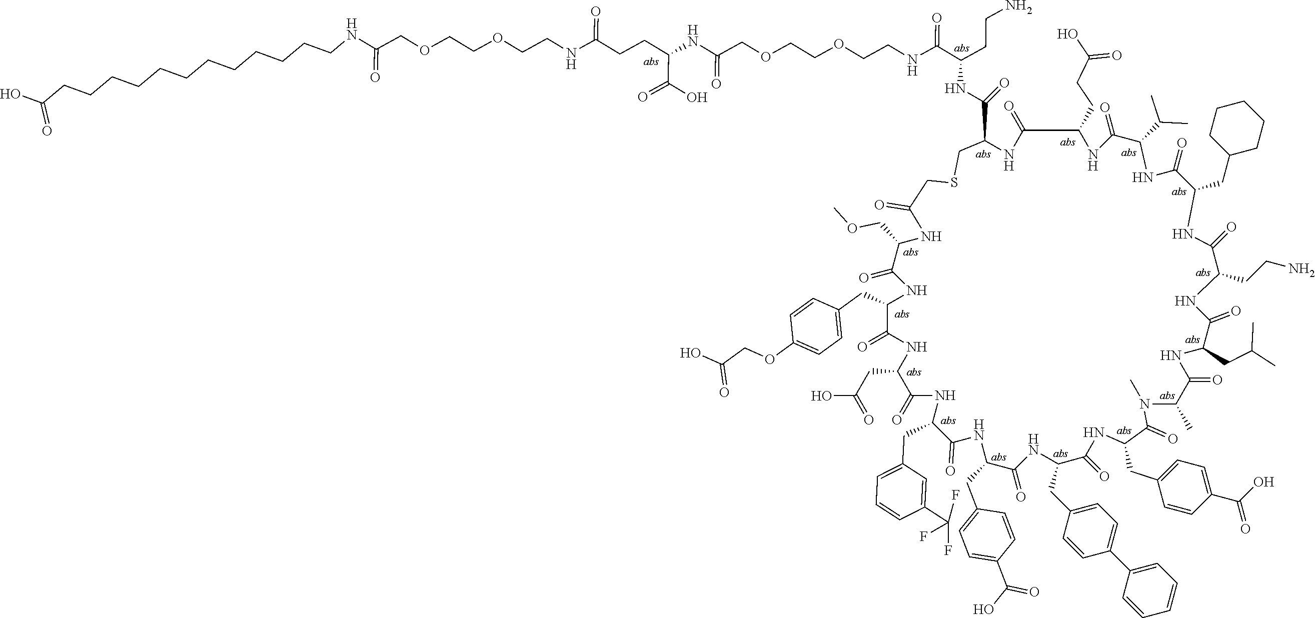 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1169 was prepared on a 50 μmol scale. The yield of the product was 13.4 mg, and its estimated purity by LCMS analysis was 96.8%. Analysis condition A: Retention time=1.55 min; ESI-MS(+) m/z [M+2H]²⁺: 1416.1.

### Preparation of Example 1170

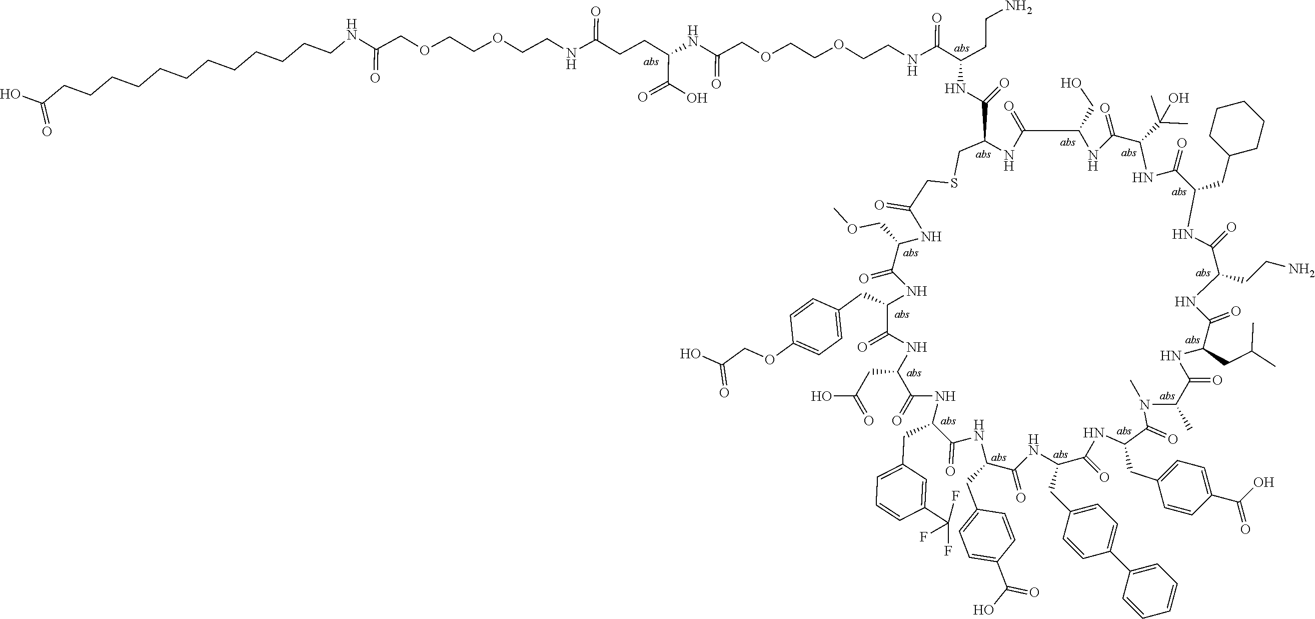 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1170 was prepared on a 50 μmol scale. The yield of the product was 13.7 mg, and its estimated purity by LCMS analysis was 100%. Analysis condition A: Retention time=1.65 min; ESI-MS(+) m/z [M+2H]²⁺: 1403.

### Preparation of Example 1171

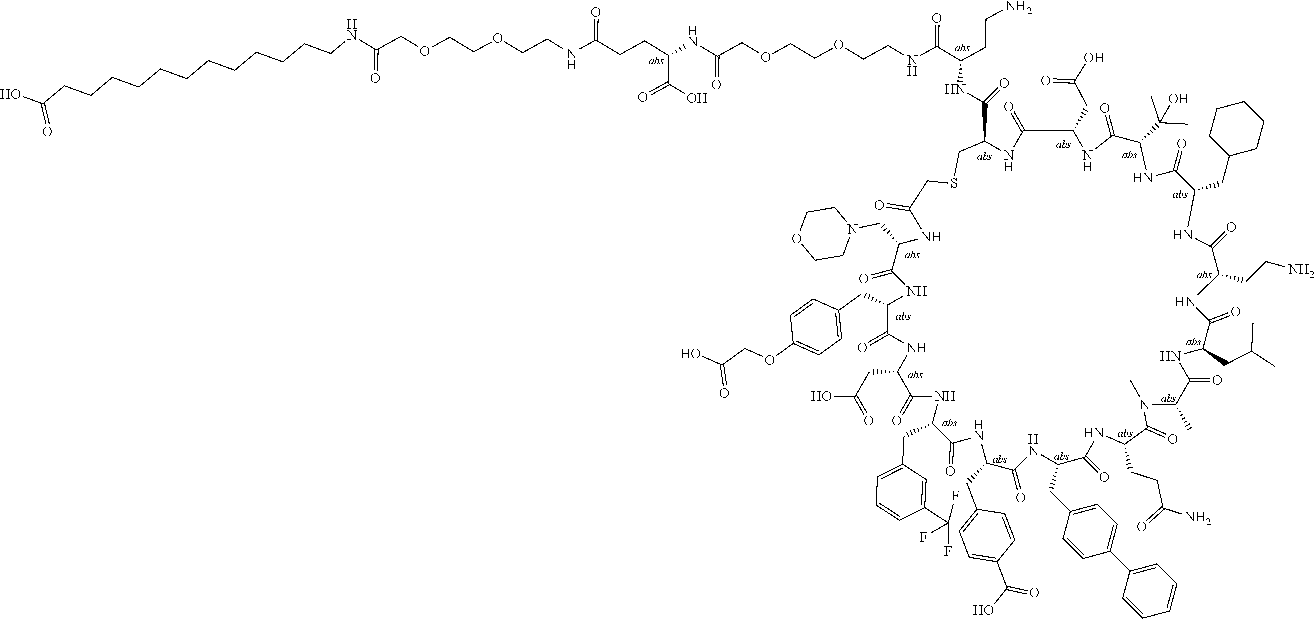 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1171 was prepared on a 50 μmol scale. The yield of the product was 10.7 mg, and its estimated purity by LCMS analysis was 96.3%. Analysis condition A: Retention time=1.6 min; ESI-MS(+) m/z [M+2H]²⁺: 1413.

### Preparation of Example 1172

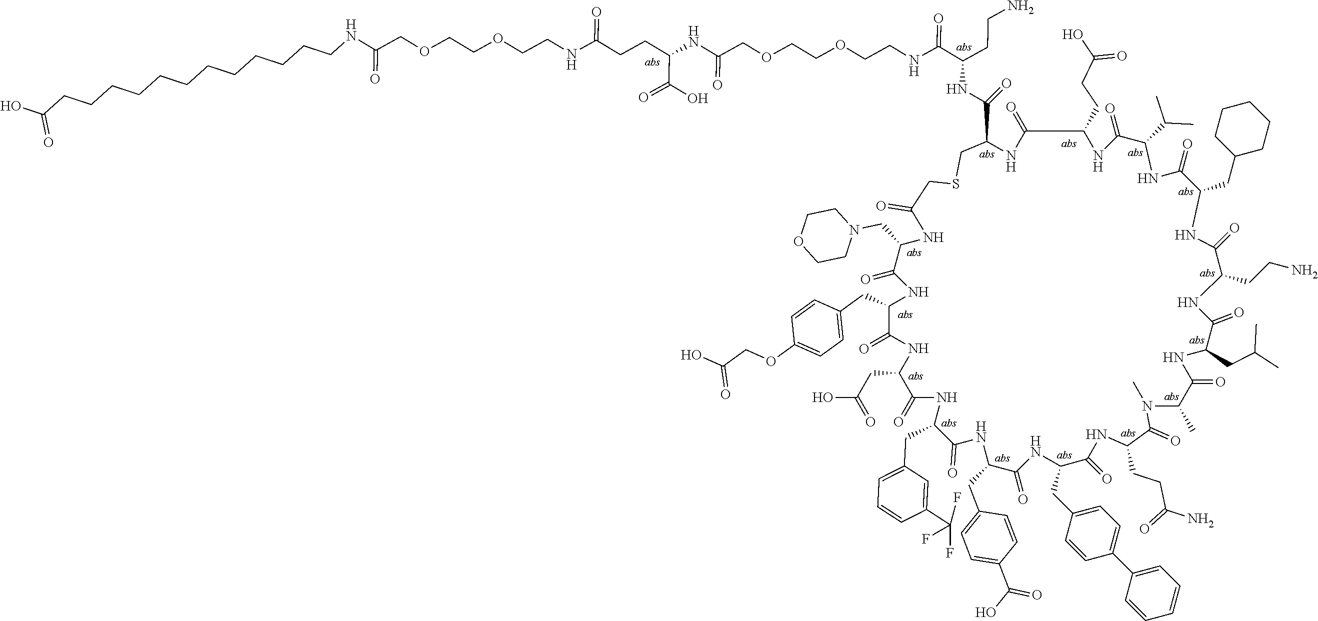 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1172 was prepared on a 50 μmol scale. The yield of the product was 7.7 mg, and its estimated purity by LCMS analysis was 97.8%. Analysis condition A: Retention time=1.63 min; ESI-MS(+) m/z [M+3H]³⁺: 941.1.

### Preparation of Example 1173

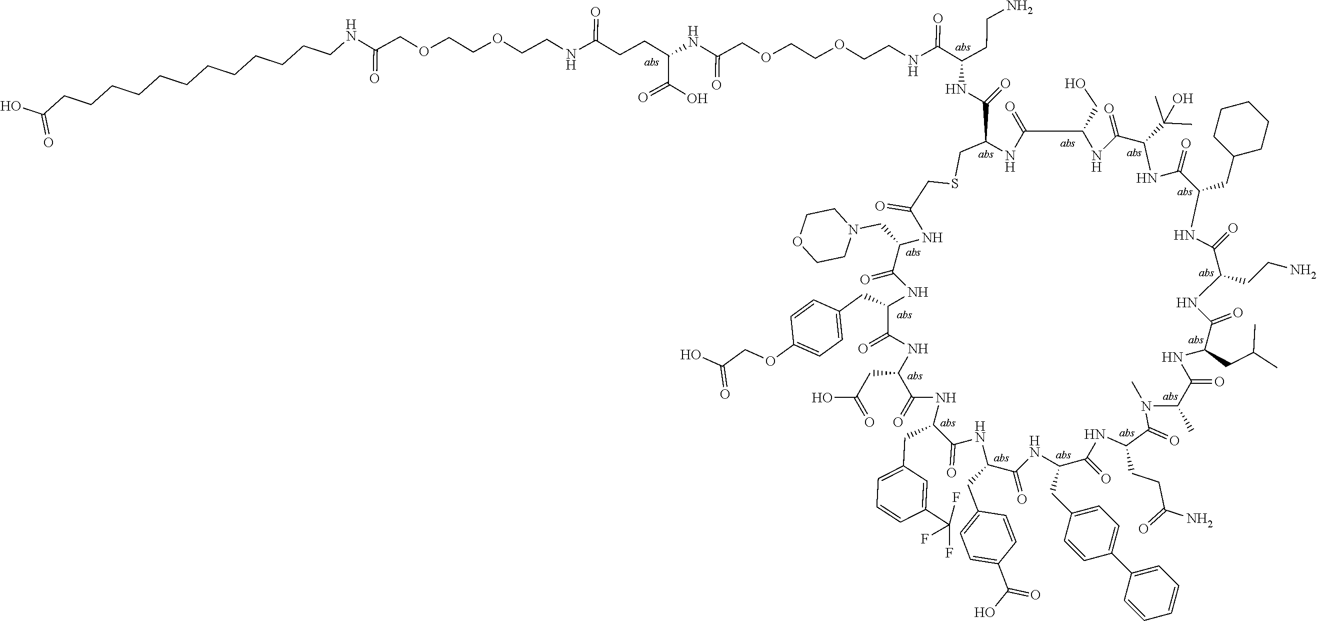 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1173 was prepared on a 50 μmol scale. The yield of the product was 6.2 mg, and its estimated purity by LCMS analysis was 100%. Analysis condition A: Retention time=1.63 min; ESI-MS(+) m/z [M+2H]²⁺: 1398.

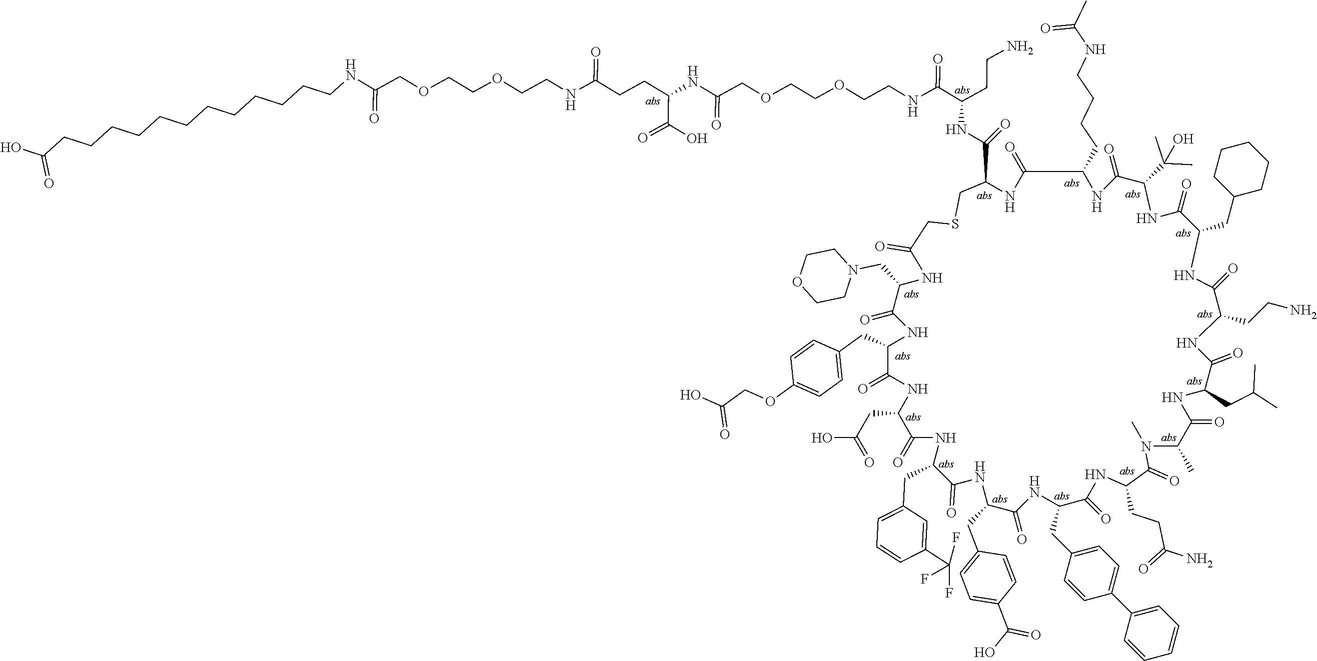 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 1174 was prepared on a 50 μmol scale. The yield of the product was 15.3 mg, and its estimated purity by LCMS analysis was 96.5%. Analysis condition A: Retention time=1.67 min; ESI-MS(+) m/z [M+2H]²⁺: 1440.

### Preparation of Example 1175

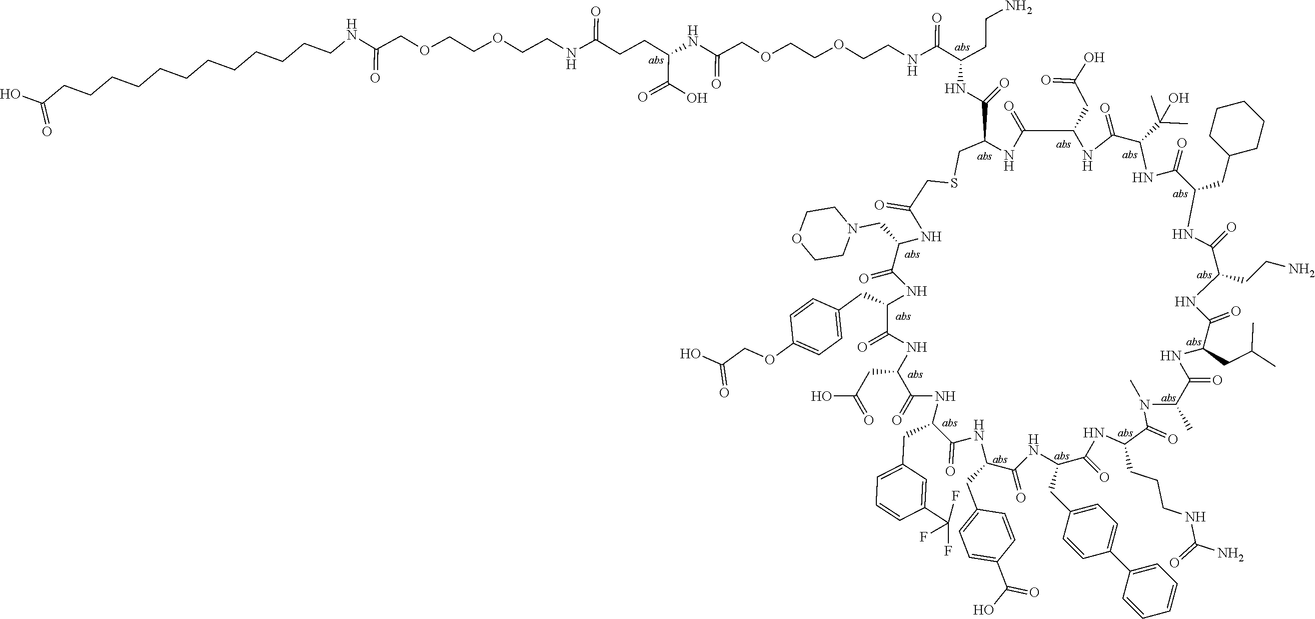 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1175 was prepared on a 50 μmol scale. The yield of the product was 9.9 mg, and its estimated purity by LCMS analysis was 98.9%. Analysis condition A: Retention time=1.65 min; ESI-MS(+) m/z [M+2H]²⁺: 1427.1.

### Preparation of Example 1176

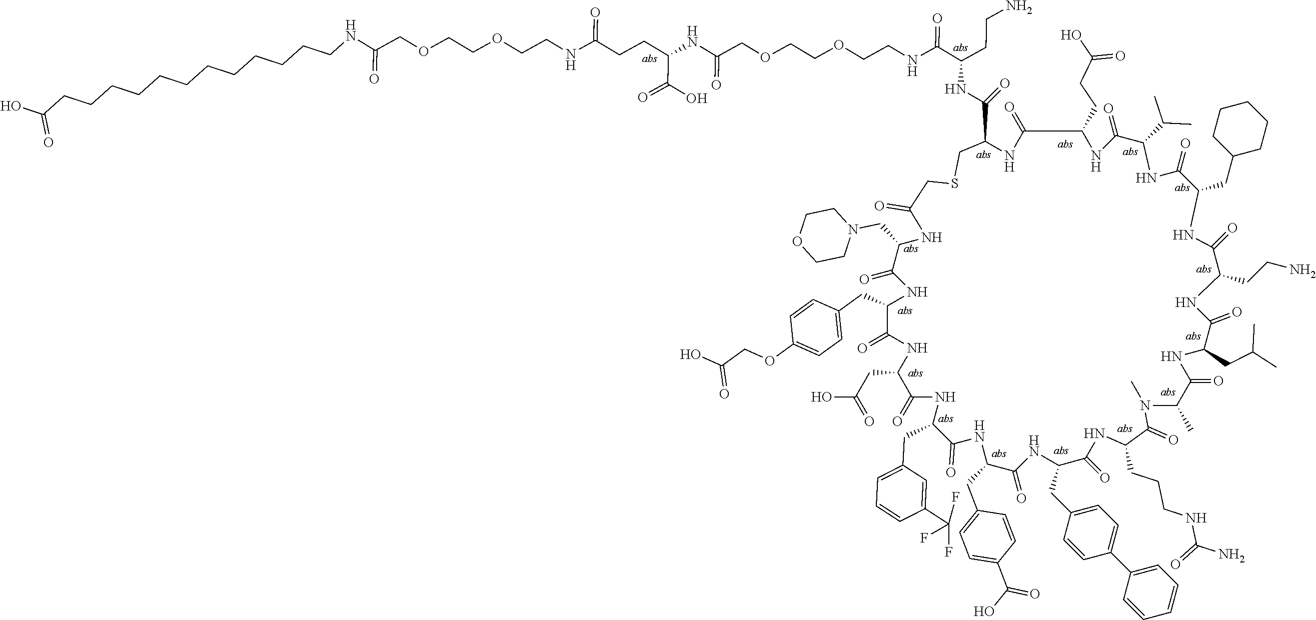 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1176 was prepared on a 50 μmol scale. The yield of the product was 3.8 mg, and its estimated purity by LCMS analysis was 93.6%. Analysis condition A: Retention time=1.9 min; ESI-MS(+) m/z [M+2H]²⁺: 1425.9.

### Preparation of Example 1177

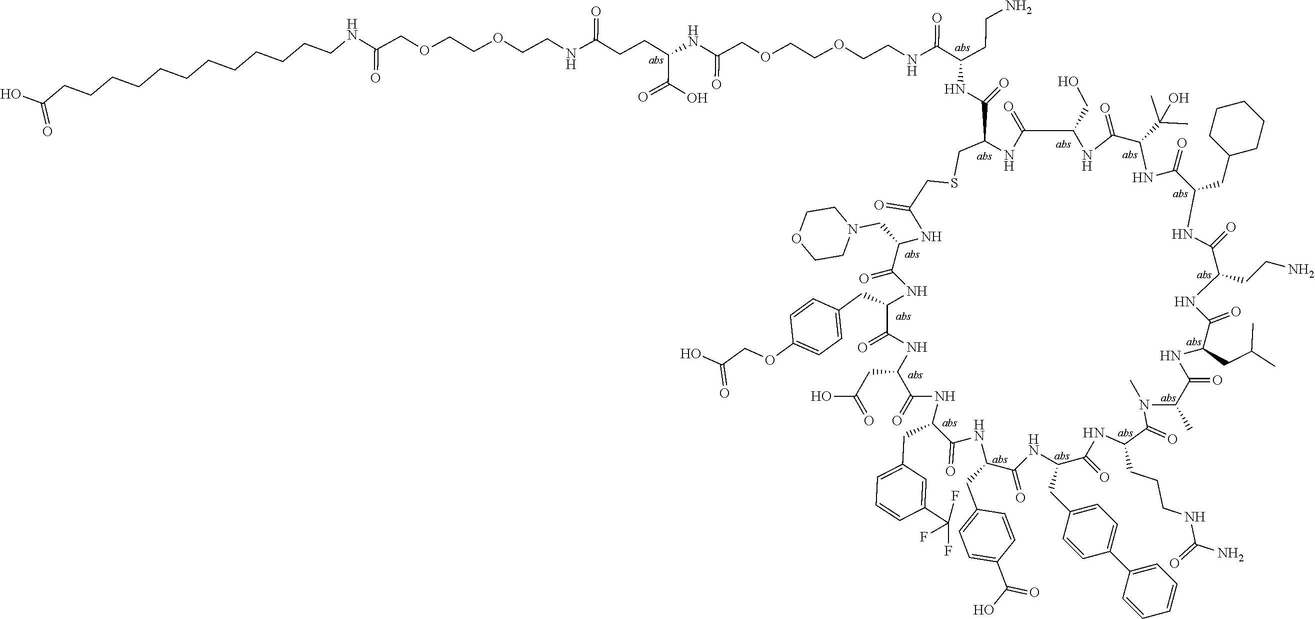 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1177 was prepared on a 50 μmol scale. The yield of the product was 6.2 mg, and its estimated purity by LCMS analysis was 92.3%. Analysis condition A: Retention time=1.64 min; ESI-MS(+) m/z [M+2H]²⁺: 1413.1.

### Preparation of Example 1178

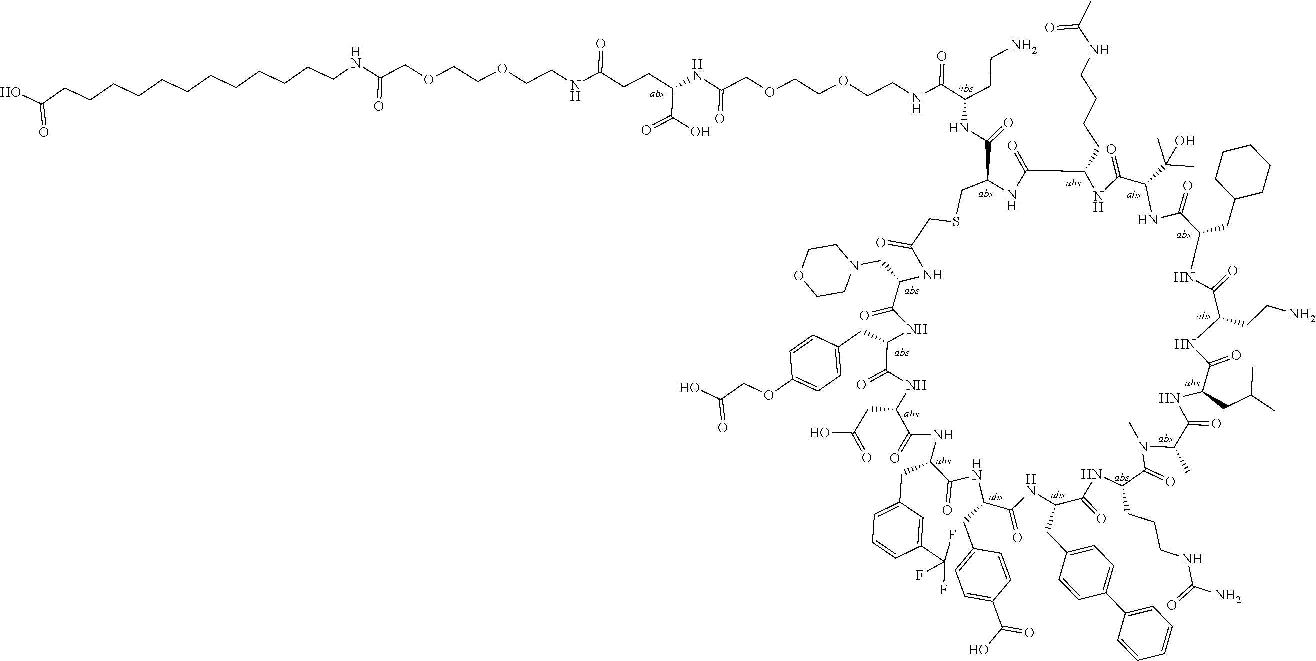

Example 1178 was prepared on a 50 μmol scale. The yield of the product was 11.4 mg, and its estimated purity by LCMS analysis was 95.9%. Analysis condition A: Retention time=1.65 min; ESI-MS (+) m/z [M+2H]²⁺: 1455.1.

### Preparation of Example 1179

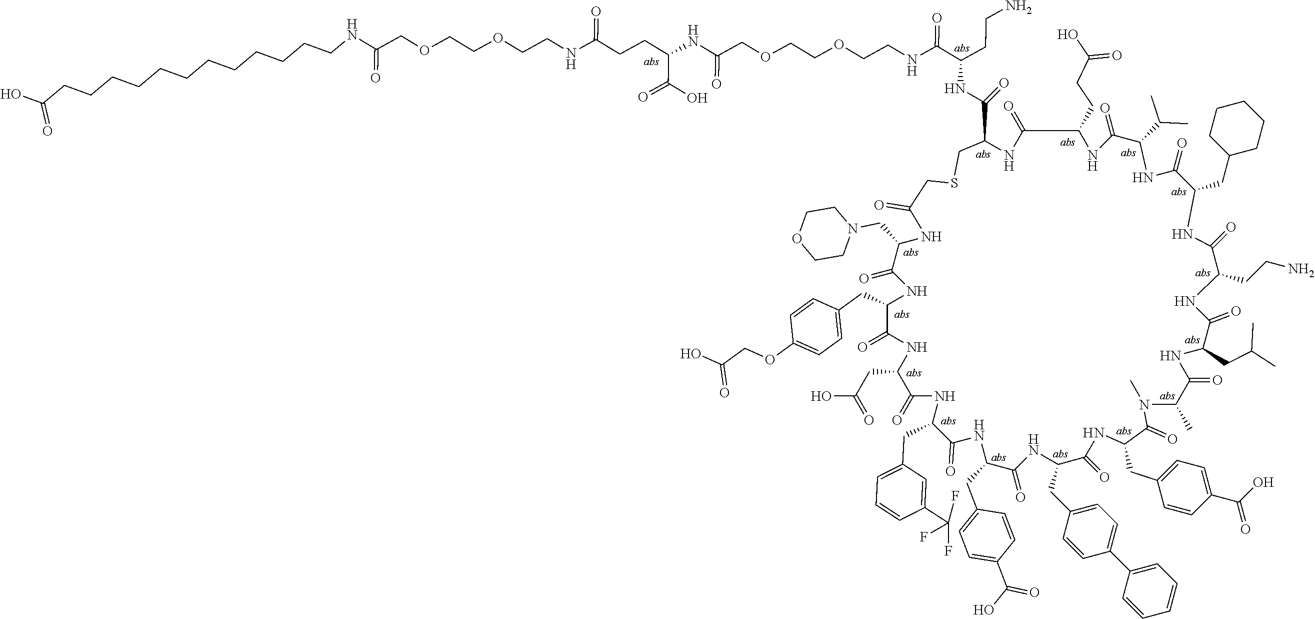 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1179 was prepared on a 50 μmol scale. The yield of the product was 9.8 mg, and its estimated purity by LCMS analysis was 97%. Analysis condition A: Retention time=1.54 min; ESI-MS(+) m/z [M+3H]³⁺: 963.

### Preparation of Example 1180

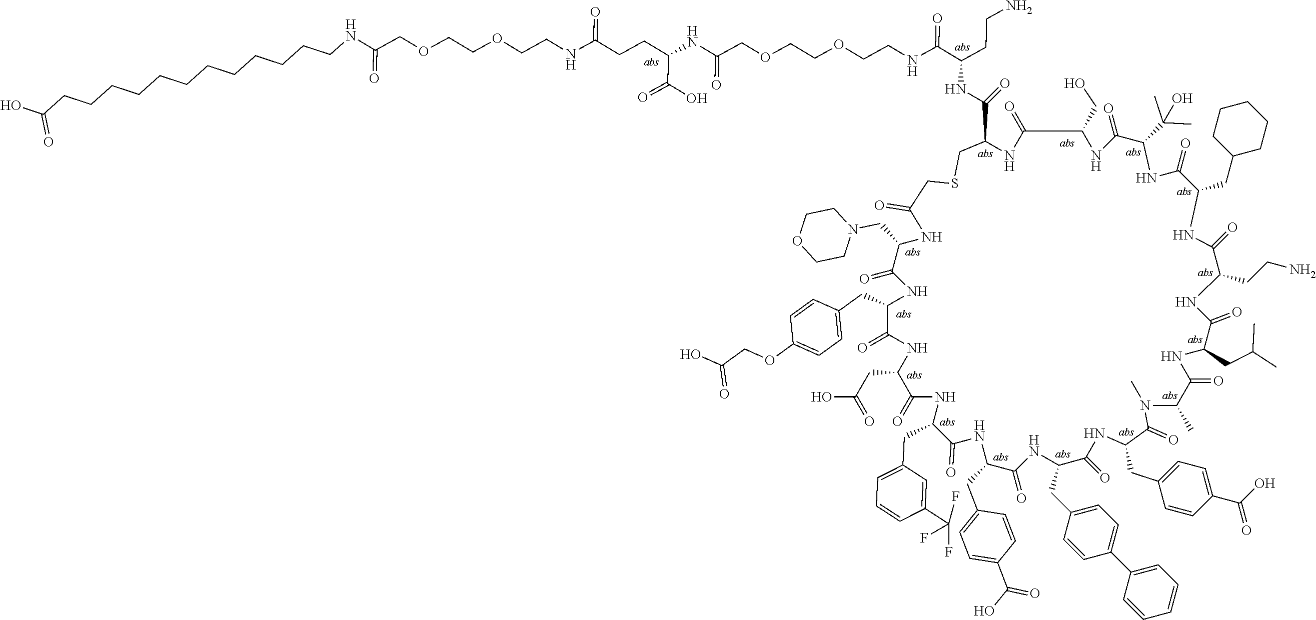 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1180 was prepared on a 50 μmol scale. The yield of the product was 25.9 mg, and its estimated purity by LCMS analysis was 100%. Analysis condition A: Retention time=1.59 min; ESI-MS(+) m/z [M+2H]²⁺: 1430.2.

### Preparation of Example 1181

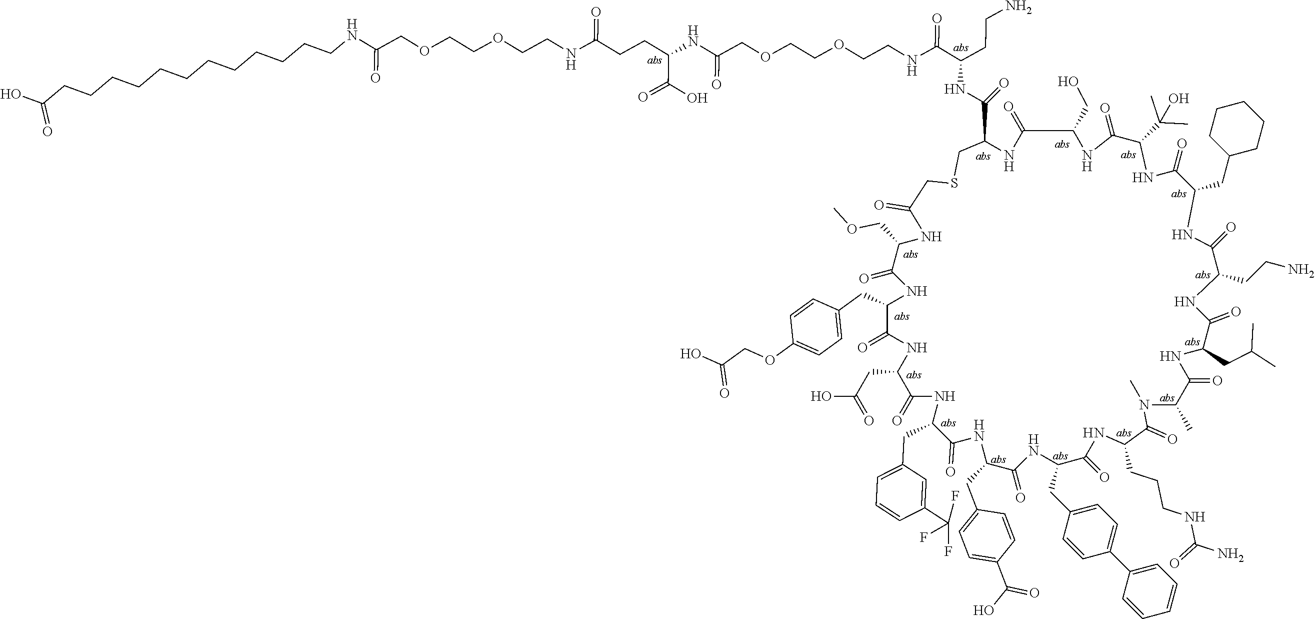 [SMILES: COC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1181 was prepared on a 50 μmol scale. The yield of the product was 15.9 mg, and its estimated purity by LCMS analysis was 91.6%. Analysis condition A: Retention time=1.63 min; ESI-MS(+) m/z [M+2H]²⁺: 1386.1.

### Preparation of Example 1182

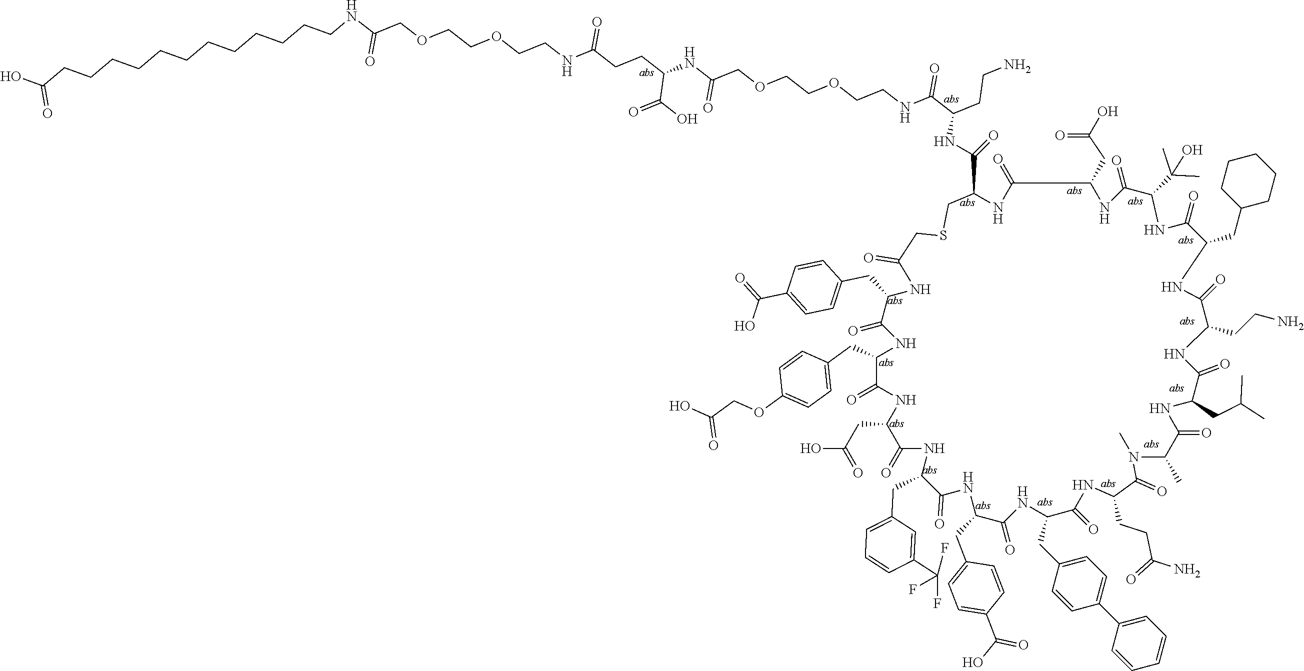 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1182 was prepared on a 50 μmol scale. The yield of the product was 39.5 mg, and its estimated purity by LCMS analysis was 94.1%. Analysis condition A: Retention time=1.5 min; ESI-MS(+) m/z [M+2H]²⁺: 1430.1.

### Preparation of Example 1183

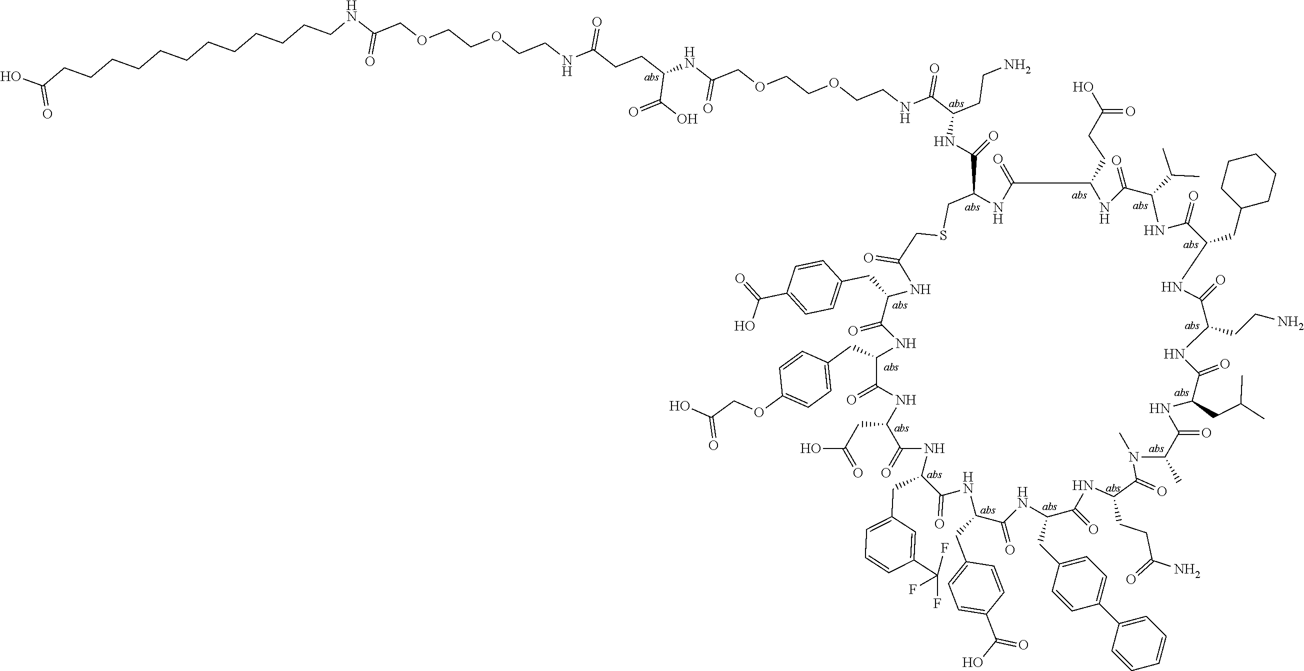 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1183 was prepared on a 50 μmol scale. The yield of the product was 5.2 mg, and its estimated purity by LCMS analysis was 96.8%. Analysis condition B: Retention time=1.96 mi; ESI-MS(+) m/z [M+2H]²⁺: 1429.1.

### Preparation of Example 1184

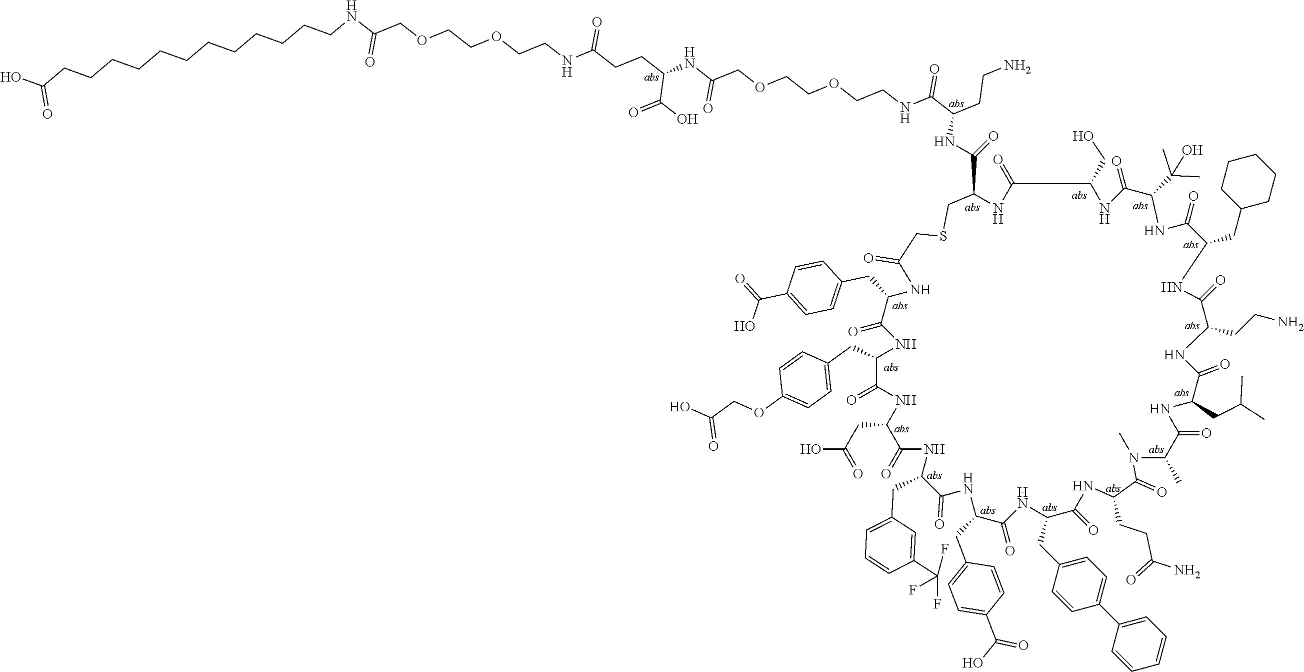 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1184 was prepared on a 50 μmol scale. The yield of the product was 15.9 mg, and its estimated purity by LCMS analysis was 93.8%. Analysis condition B: Retention time=1.97 min; ESI-MS(+) m/z [M+2H]²⁺: 1416.5.

### Preparation of Example 1185

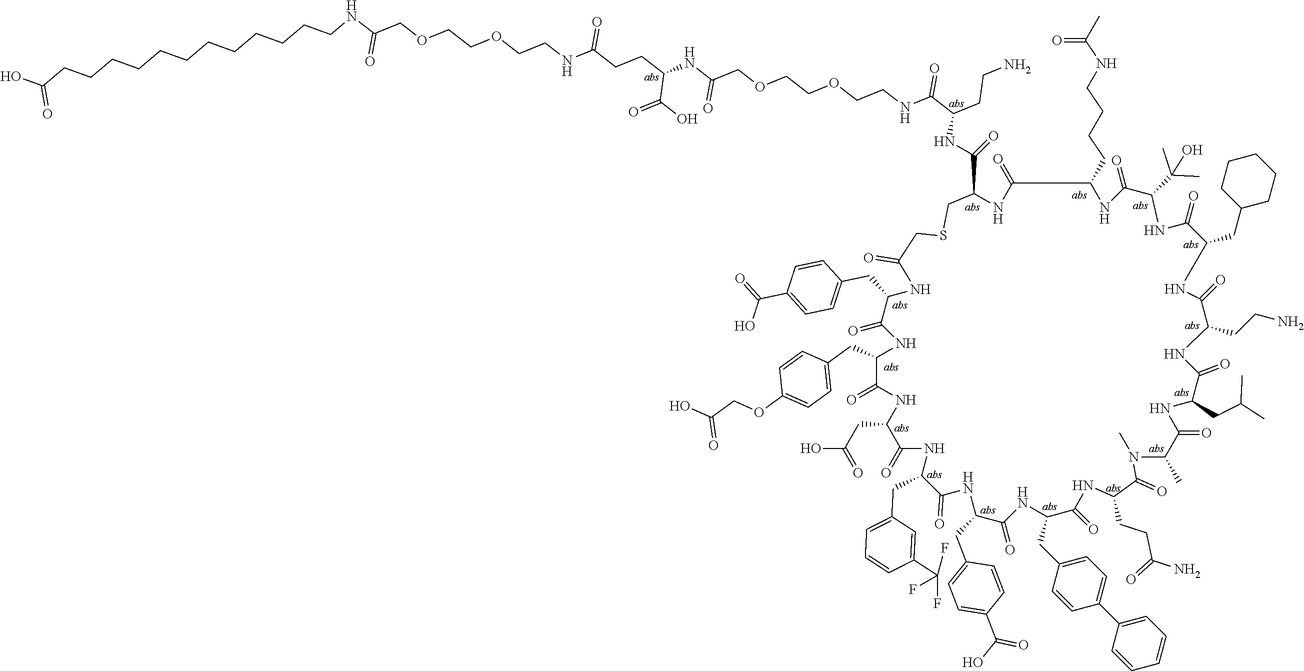 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 1185 was prepared on a 50 μmol scale. The yield of the product was 43.5 mg, and its estimated purity by L CMS analysis was 100%. Analysis condition B: Retention time=−2.02 min; ESI-MS(+) m/z [M+2H]²⁺: 1458.

### Preparation of Example 1186

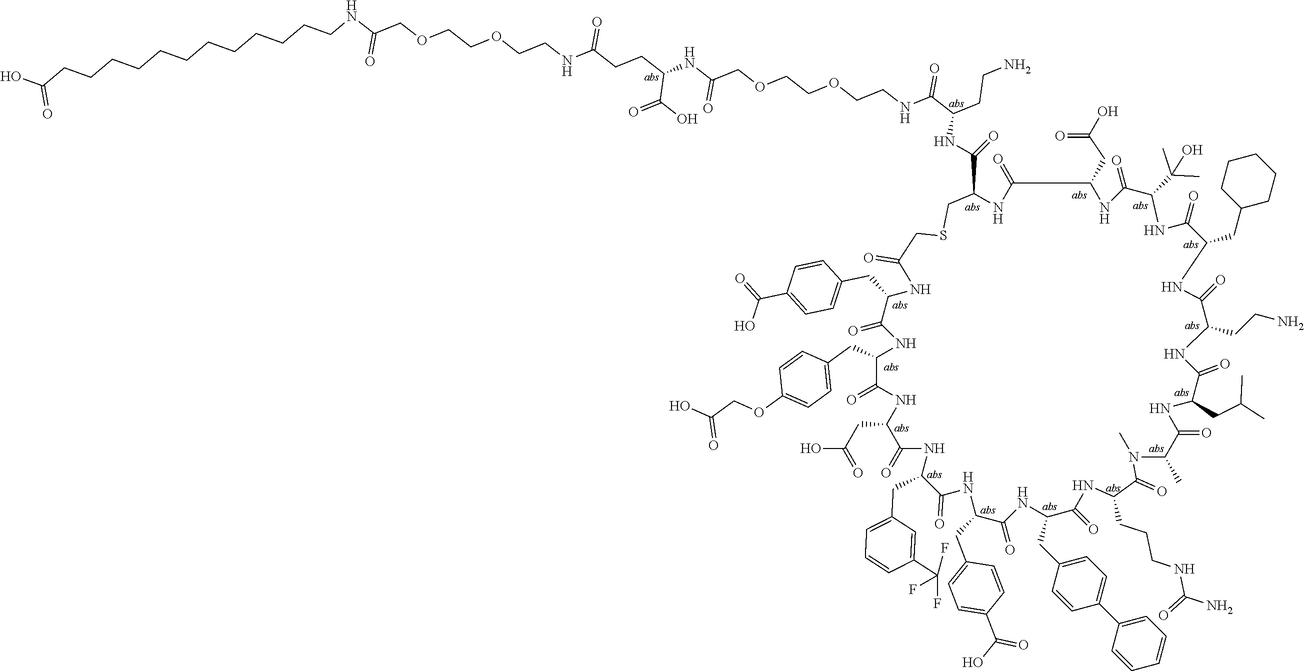 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Example 1186 was prepared on a 50 μmol scale. The yield of the product was 143.5 mg, and its estimated purity by LCMS analysis was 97%. Analysis condition B: Retention time=2.07 min; ESI-MS(+) m/z [M+2H]²⁺: 14458.1

### Preparation of Example 1187

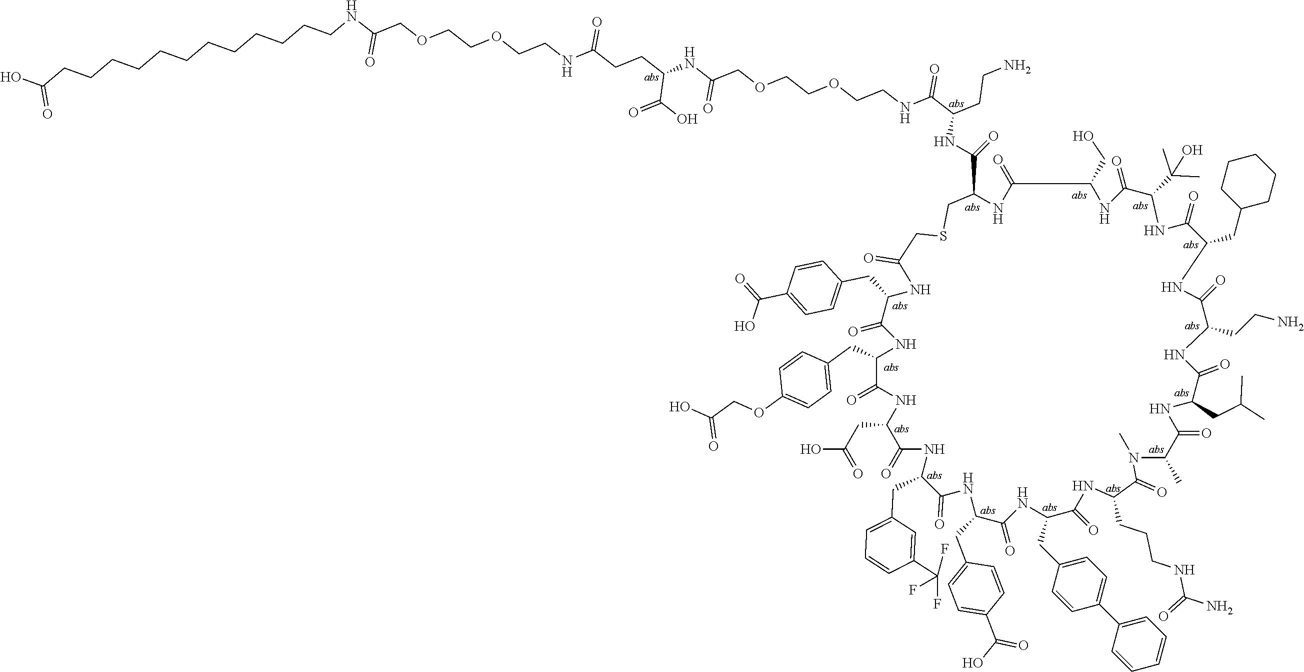 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1187 was prepared on a 50 μmol scale. The yield of the product was 16.2 mg, and its estimated purity by LCMS analysis was 95.9%. Analysis condition A: Retention time=1.7 min; ESI-MS(+) m/z [M+2H]²⁺: 1430.9.

### Preparation of Example 1188

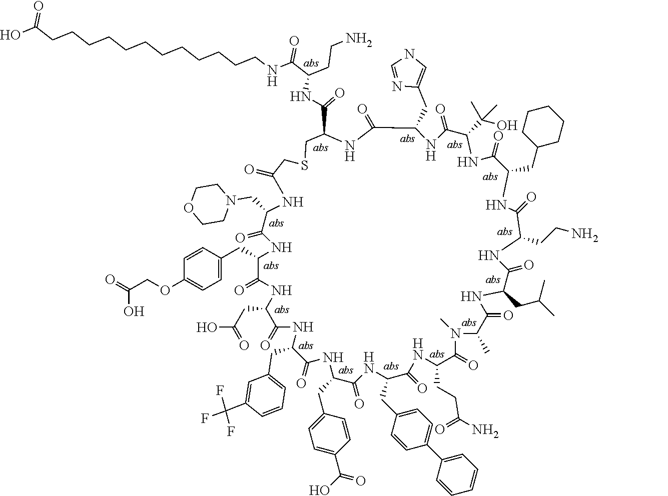 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1188 was prepared on a 50 μmol scale. The yield of the product was 21.3 mg, and its estimated purity by LCMS analysis was 97.3%. Analysis condition A: Retention time=1.79 min; ESI-MS(+) m/z [M+2H]²⁺: 1214.

### Preparation of Example 1189

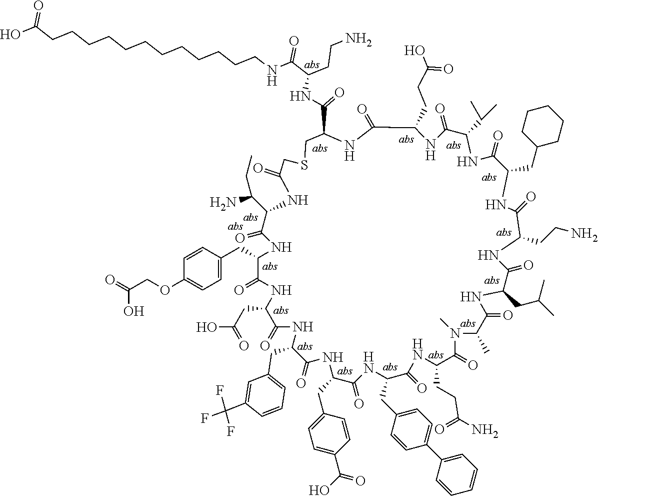 [SMILES: CC[C@H](N)[C@@H]1NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 1189 was prepared on a 50 μmol scale. The yield of the product was 15 mg, and its estimated purity by LCMS analysis was 92.6%. Analysis condition A: Retention time=1.61 min; ESI-MS(+) m/z [M+2H]²⁺: 1174.

### Preparation of Example 1190

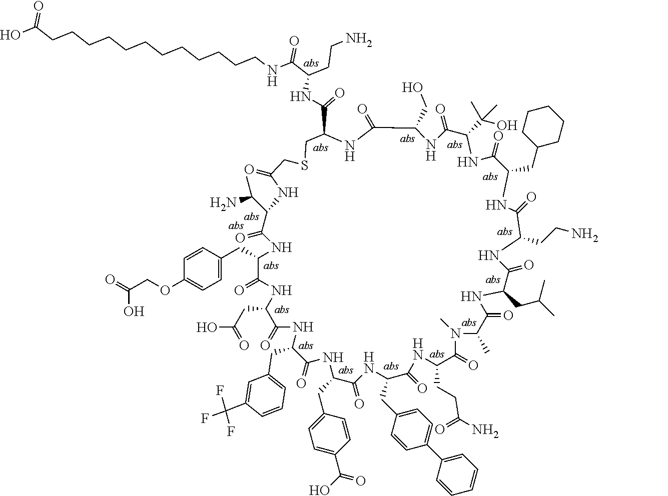 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 1190 was prepared on a 50 μmol scale. The yield of the product was 19.4 mg, and its estimated purity by LCMS analysis was 92%. Analysis condition A: Retention time=1.66 min; ESI-MS(+) m/z [M+2H]²⁺: 1160.2.

### Preparation of Example 1191

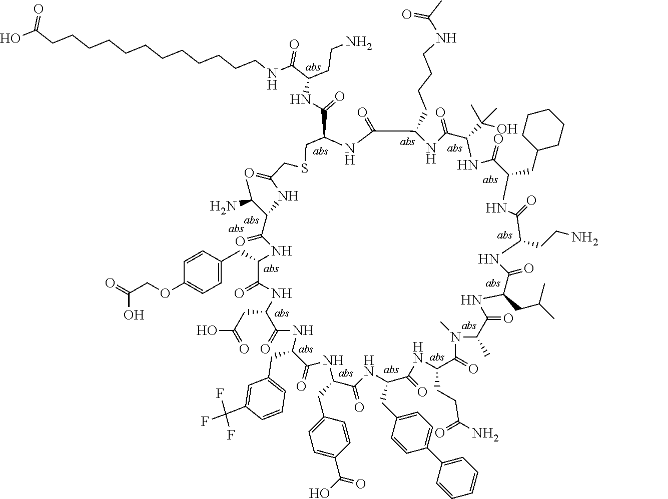 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC1=O]

Example 1191 was prepared on a 50 μmol scale. The yield of the product was 50.8 mg, and its estimated purity by LCMS analysis was 96.1%. Analysis condition A: Retention time=1.66 min; ESI-MS(+) m/z [M+2H]²⁺: 1202.2.

### Preparation of Example 1192

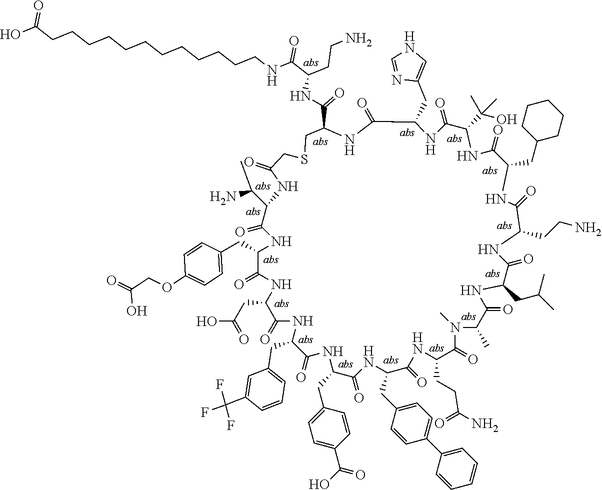 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1192 was prepared on a 50 μmol scale. The yield of the product was 26.1 mg, and its estimated purity by LCMS analysis was 92.1%. Analysis condition A: Retention time=1.72 min; ESI-MS(+) m/z [M+2H]²⁺: 1186.

### Preparation of Example 1193

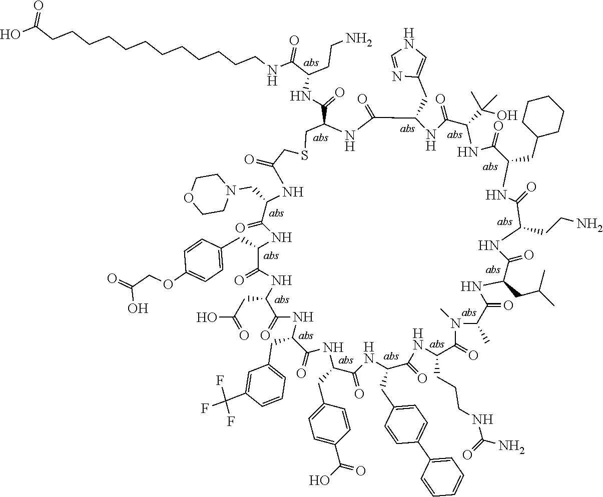 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1193 was prepared on a 50 μmol scale. The yield of the product was 24.3 mg, and its estimated purity by LCMS analysis was 99%. Analysis condition B: Retention time=1.77 min; ESI-MS(+) m/z [M+3H]³⁺: 819.4.

### Preparation of Example 1194

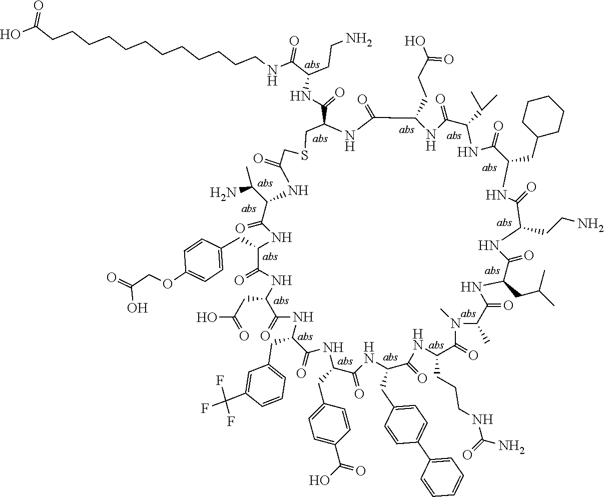 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1194 was prepared on a 50 μmol scale. The yield of the product was 11.8 mg, and its estimated purity by LCMS analysis was 90.2%. Analysis condition A: Retention time=1.63 min; ESI-MS(+) m/z [M+2H]²⁺: 1188.3.

### Preparation of Example 1195

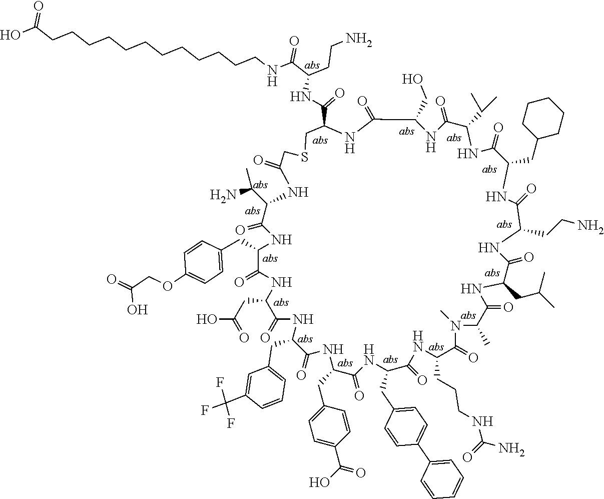 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 1195 was prepared on a 50 μmol scale. The yield of the product was 7.2 mg, and its estimated purity by LCMS analysis was 98.5%. Analysis condition A: Retention time=1.85 min; ESI-MS(+) m/z [M+3H]³⁺: 784.1.

### Preparation of Example 1196

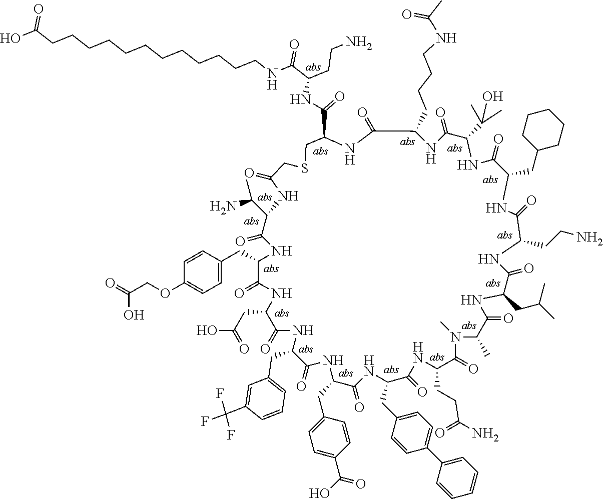 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC1=O]

Example 1196 was prepared on a 50 μmol scale. The yield of the product was 30 mg, and its estimated purity by LCMS analysis was 91.3%. Analysis condition A: Retention time=1.71 min; ESI-MS(+) m/z [M+2H]²⁺: 1217.1.

### Preparation of Example 1197

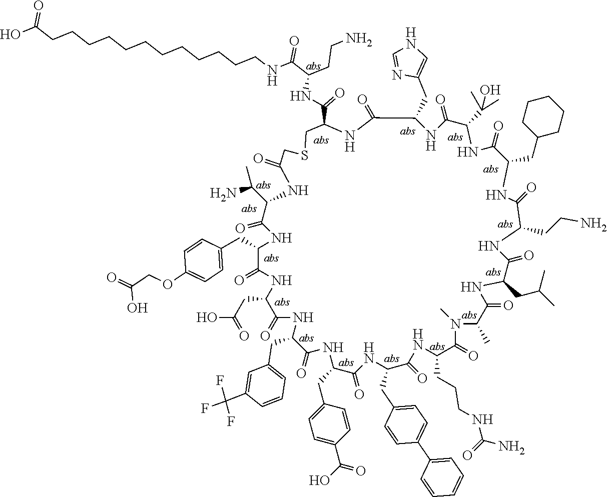 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1197 was prepared on a 50 μmol scale. The yield of the product was 13.6 mg, and its estimated purity by LCMS analysis was 96%. Analysis condition B: Retention time=1.79 min; ESI-MS(+) m/z [M+3H]³⁺: 801.1.

### Preparation of Example 1198

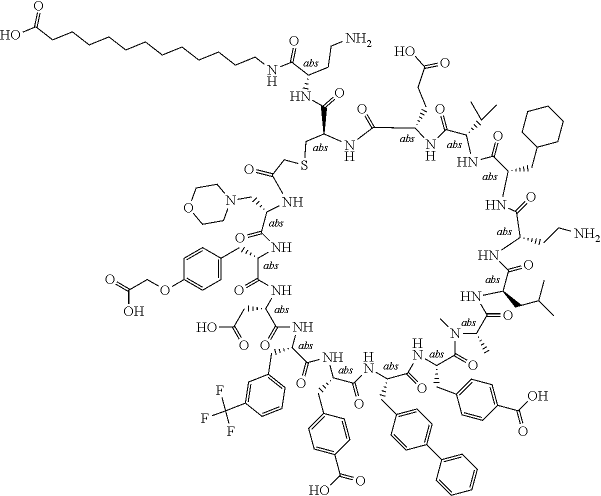 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1198 was prepared on a 50 μmol scale. The yield of the product was 12.5 mg, and its estimated purity by LCMS analysis was 90%. Analysis condition A: Retention time=1.54 min; ESI-MS(+) m/z [M+3H]³⁺: 823.

### Preparation of Example 1199

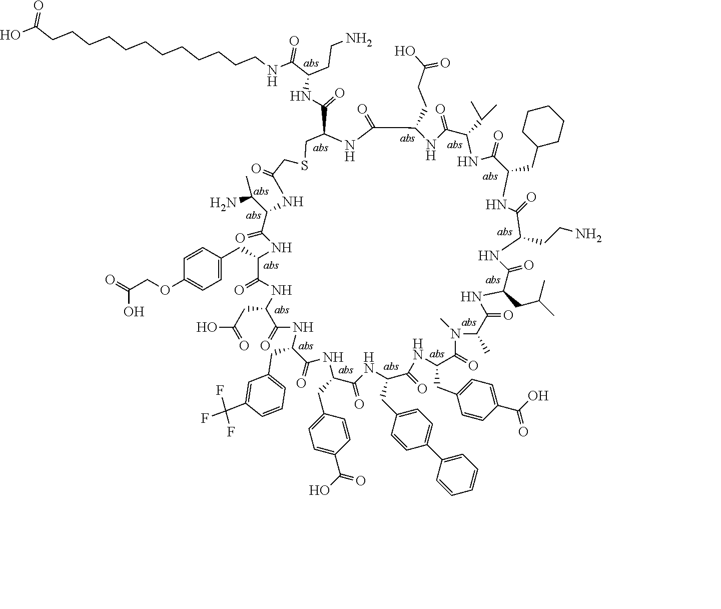 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1199 was prepared on a 50 μmol scale. The yield of the product was 17.3 mg, and its estimated purity by LCMS analysis was 91.2%. Analysis condition A: Retention time=1.58 min; ESI-MS(+) m/z [M+3H]³⁺: 804.3.

### Preparation of Example 1200

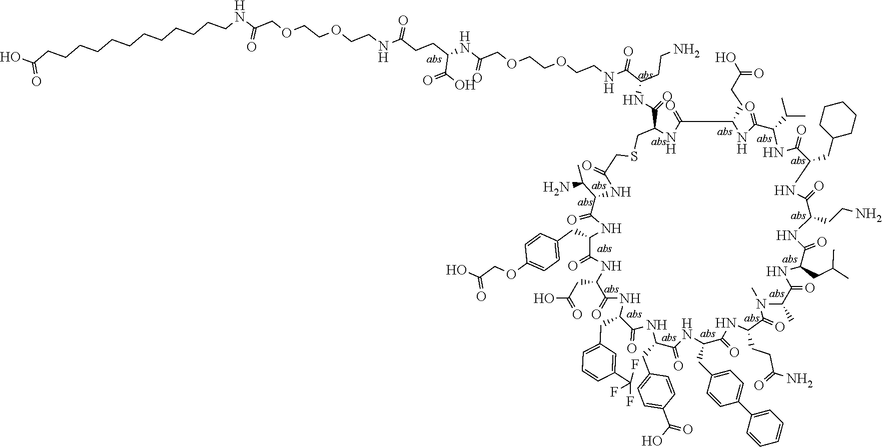 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1200 was prepared on a 50 μmol scale. The yield of the product was 7.3 mg, and its estimated purity by LCMS analysis was 90.1%. Analysis condition A: Retention time=1.64 min; ESI-MS(+) m/z [M+2H]²⁺: 1384.

### Preparation of Example 1201

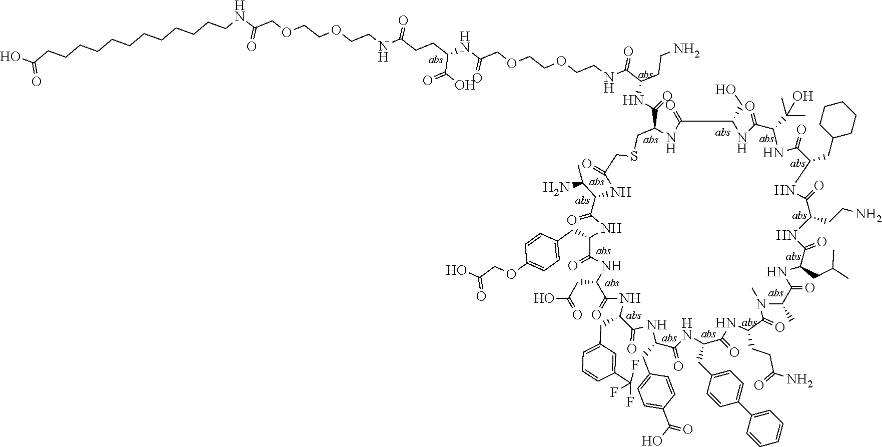 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1201 was prepared on a 50 μmol scale. The yield of the product was 41.1 mg, and its estimated purity by LCMS analysis was 93.4%. Analysis condition A: Retention time=1.65 min; ESI-MS(+) m/z [M+3H]²⁺: 913.9.

### Preparation of Example 1202

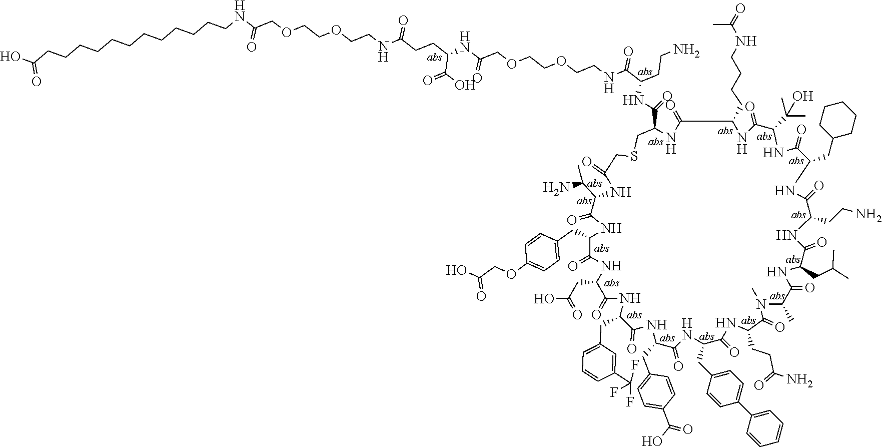 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 1202 was prepared on a 50 μmol scale. The yield of the product was 38.6 mg, and its estimated purity by LCMS analysis was 90.3%. Analysis condition A: Retention time=1.85 min; ESI-MS(+) m/z [M+2H]²⁺: 1411.9.

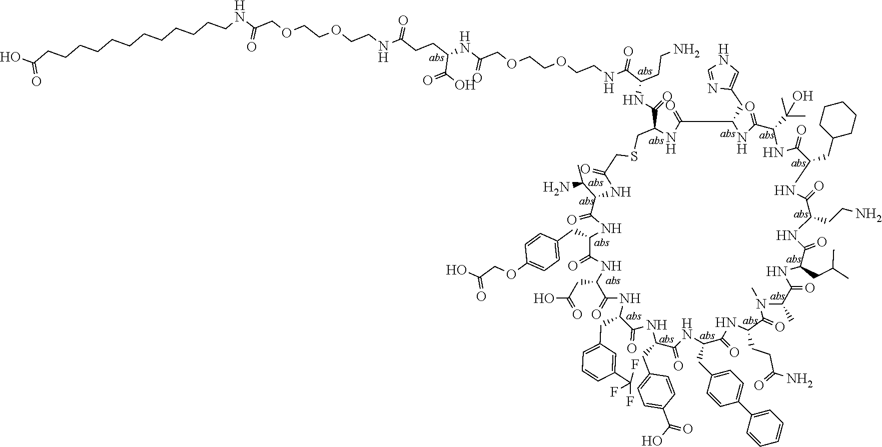 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Example 1203 was prepared on a 50 μmol scale. The yield of the product was 9.9 mg, and its estimated purity by LCMS analysis was 98.4%. Analysis condition A: Retention time=1.7 min; ESI-MS(+) m/z [M+2H]²⁺: 1396.1.

### Preparation of Example 1204

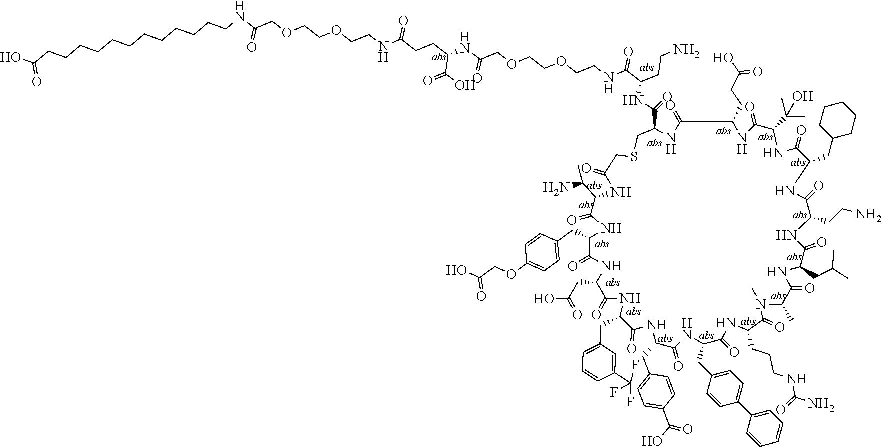 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 1204 was prepared on a 50 μmol scale. The yield of the product was 15 mg, and its estimated purity by LCMS analysis was 97%. Analysis condition A: Retention time=1.95 min; ESI-MS(+) m/z [M+2H]²⁺: 1398.

### Preparation of Example 1205

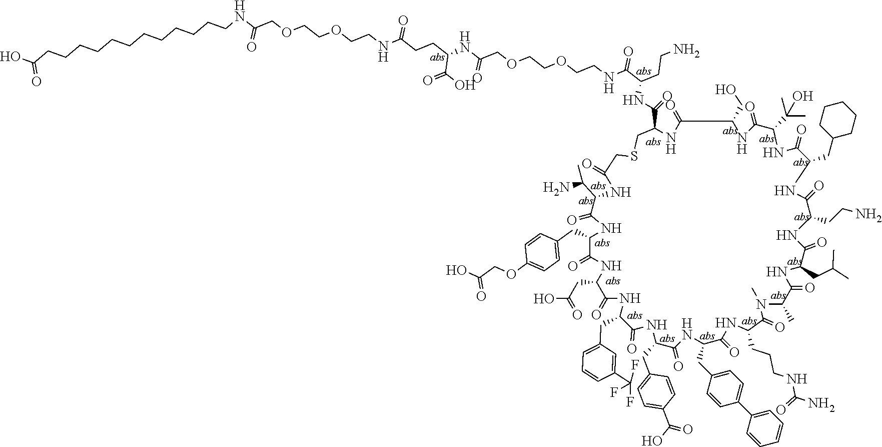 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Example 1205 was prepared on a 50 μmol scale. The yield of the product was 8.3 mg, and its estimated purity by LCMS analysis was 95%. Analysis condition A: Retention time=1.71 min; ESI-MS(+) m/z [M+2H]²⁺: 1385.

### Preparation of Example 1206

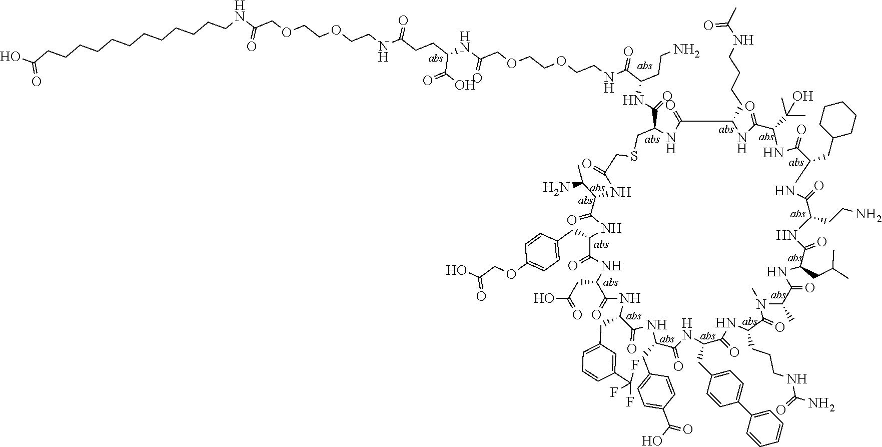 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 1206 was prepared on a 50 μmol scale. The yield of the product was 43.3 mg, and its estimated purity by LCMS analysis was 91.3%. Analysis condition A: Retention time=1.64 min; ESI-MS(+) m/z [M+2H]²⁺: 1426.8.

### Preparation of Example 1207

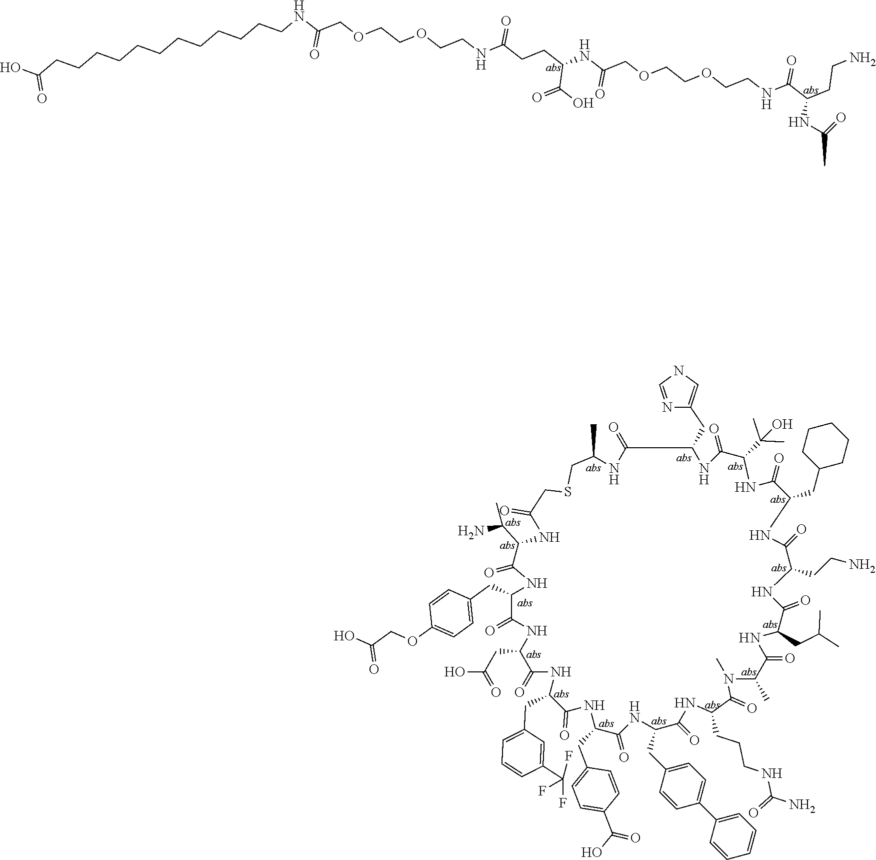

Example 1207 was prepared on a 50 μmol scale. The yield of the product was 7 mg, and its estimated purity by LCMS analysis was 91.3%. Analysis condition A: Retention time=1.79 min; ESI-MS(+) m/z [M+2H]²⁺: 1410.1.

### Preparation of Example 1208

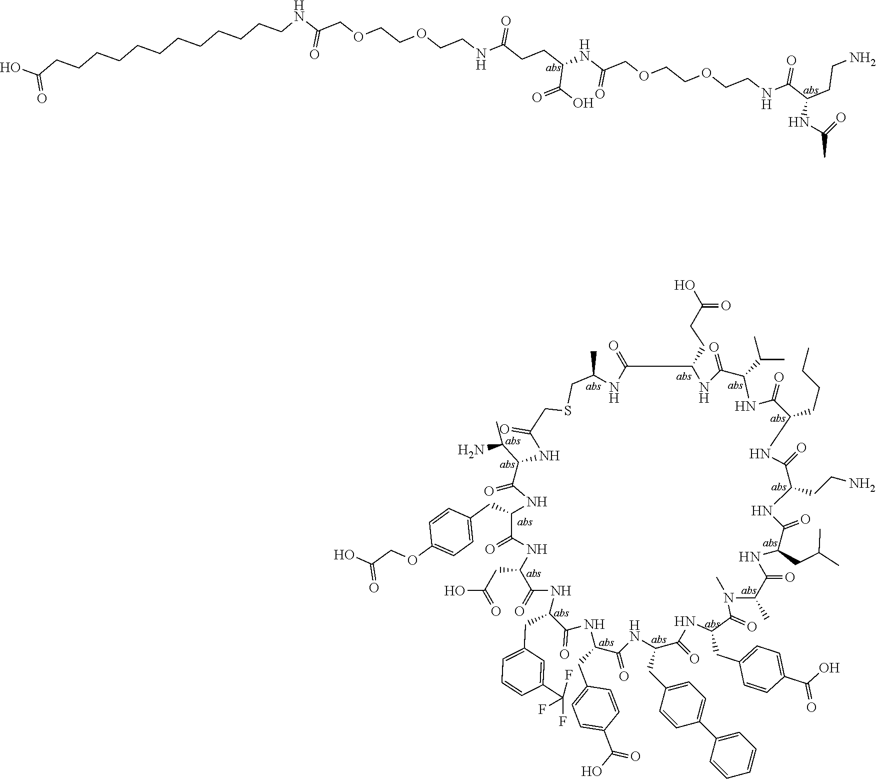

Example 1208 was prepared on a 50 μmol scale. The yield of the product was 30 mg, and its estimated purity by LCMS analysis was 94.2%. Analysis condition A: Retention time=1.55 min; ESI-MS(+) m/z [M+2H]²⁺: 1415.1.

### Preparation of Example 1209

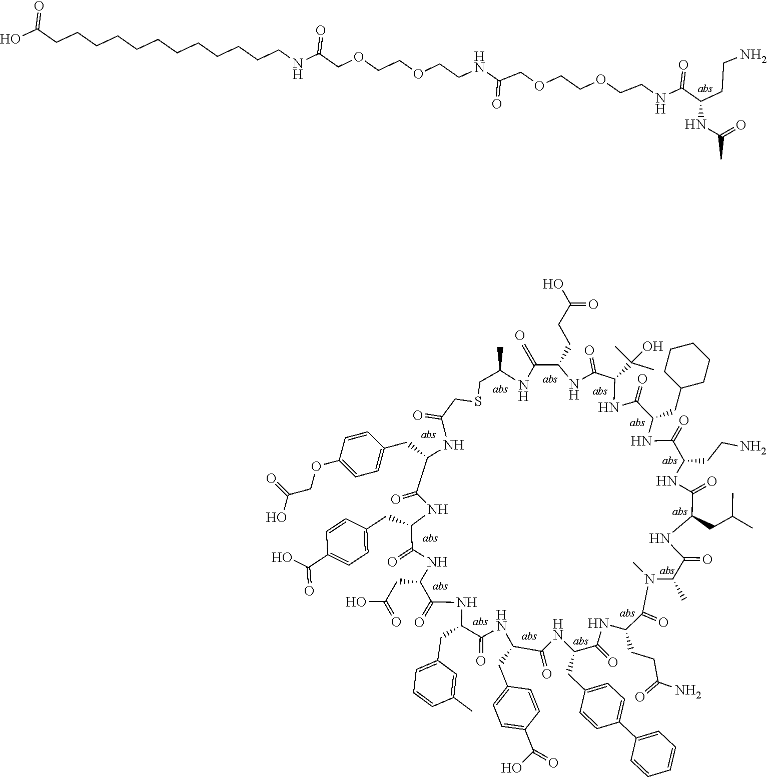

Example 1209 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 29% B, 29-69% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 36.7 mg, and its estimated purity by LCMS analysis was 95.6%. Analysis condition A: Retention time=1.59 min; ESI-MS(+) m/z [M+2H]²⁺: 1339.1.

### Preparation of Example 1210

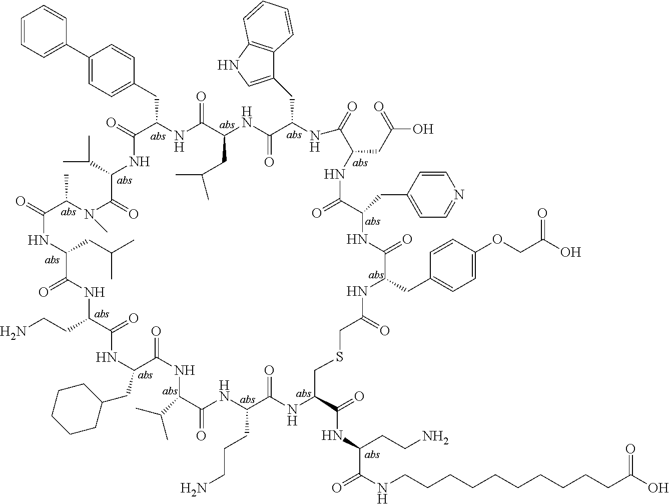 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1210 was prepared on a 50 μmol scale. The yield of the product was 7.6 mg, and its estimated purity by LCMS analysis was 85.3%. Analysis condition B: Retention time=1.62 min; ESI-MS(+) m/z [M+3H]³⁺: 739.

### Preparation of Example 1211

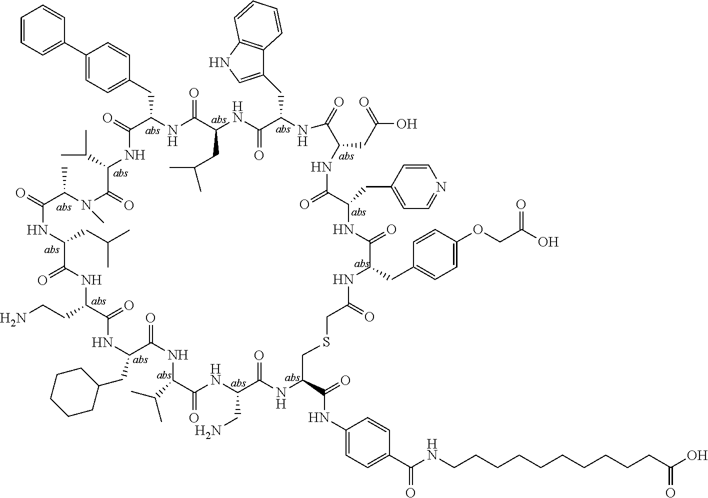 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1211 was prepared on a 50 μmol scale. The yield of the product was 18.5 mg, and its estimated purity by LCMS analysis was 88%. Analysis condition B: Retention time=1.83 min; ESI-MS(+) m/z [M+2H]²⁺: 1104.1.

### Preparation of Example 1212

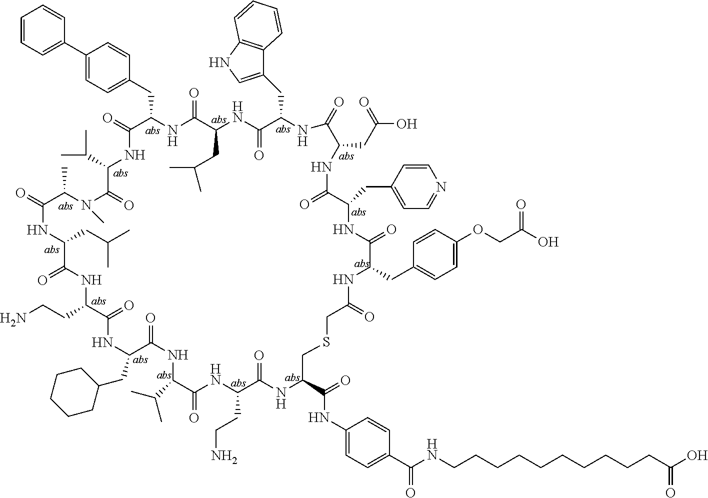 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1212 was prepared on a 50 μmol scale. The yield of the product was 8.7 mg, and its estimated purity by LCMS analysis was 94.4%. Analysis condition B: Retention time=1.72, 1.77 min; ESI-MS(+) m/z [M+3H]³⁺: 741.17, 741.17.

### Preparation of Example 1213

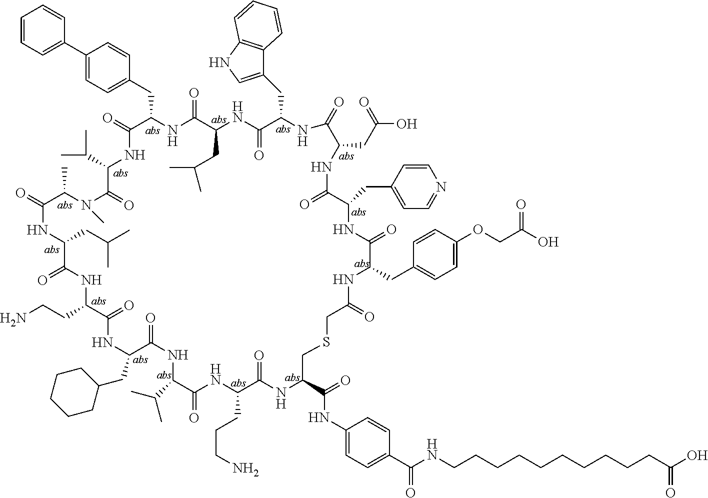 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1213 was prepared on a 50 μmol scale. The yield of the product was 22.2 mg, and its estimated purity by LCMS analysis was 91.9%. Analysis condition A: Retention time=1.86 min; ESI-MS(+) m/z [M+2H]²⁺: 1117.9.

### Preparation of Example 1214

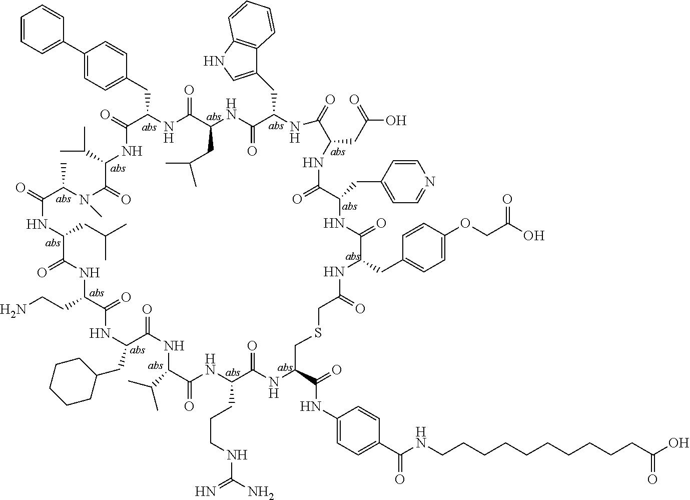 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1214 was prepared on a 50 μmol scale. The yield of the product was 16.8 mg, and its estimated purity by LCMS analysis was 84.1%. Analysis condition B: Retention time=1.69 min; ESI-MS(+) m/z [M+2H]²⁺: 1139.4.

### Preparation of Example 1215

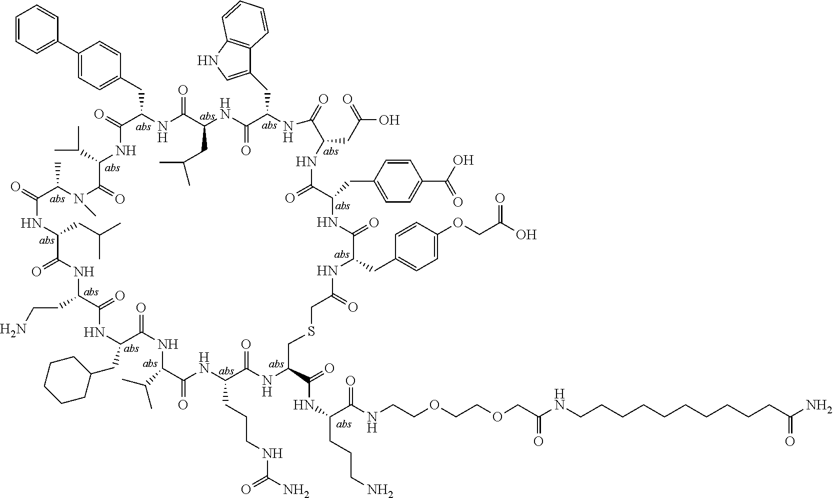 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(N)=O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1215 was prepared on a 50 μmol scale. The yield of the product was 9.1 mg, and its estimated purity by LCMS analysis was 96.6%. Analysis condition B: Retention time=1.71 min; ESI-MS(+) m/z [M+2H]²⁺: 1230.6.

### Preparation of Example 1216

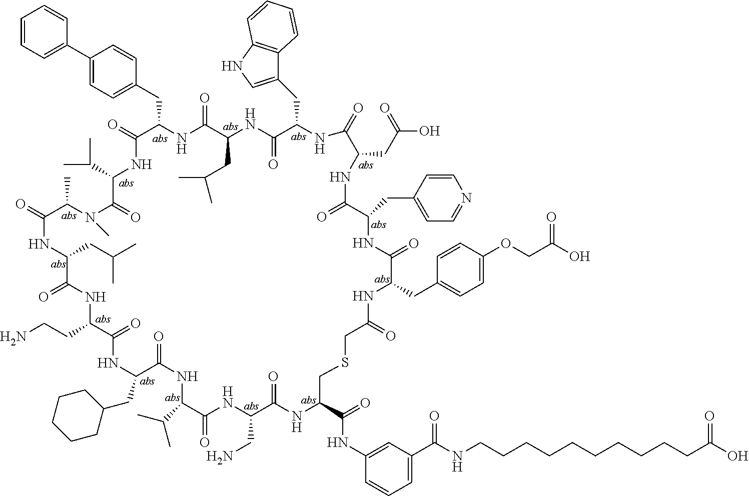 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1216 was prepared on a 50 μmol scale. The yield of the product was 16.9 mg, and its estimated purity by LCMS analysis was 90.2%. Analysis condition A: Retention time=1.96 min; ESI-MS(+) m/z [M+2H]²⁺: 1104.2.

### Preparation of Example 1217

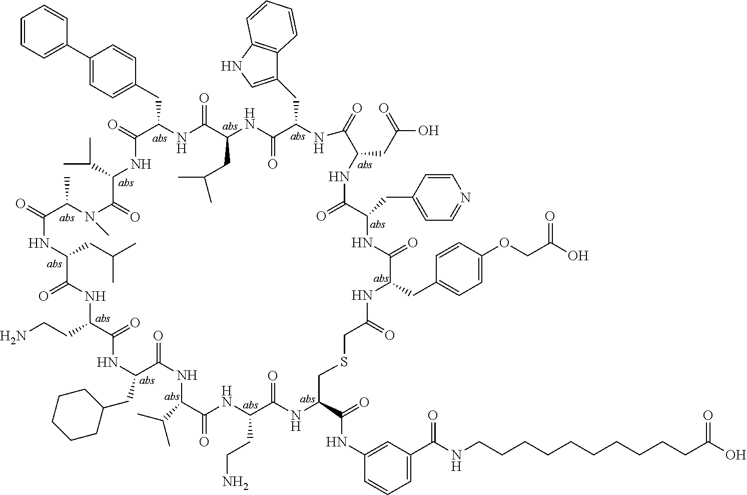 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1217 was prepared on a 50 μmol scale. The yield of the product was 6.4 mg, and its estimated purity by LCMS analysis was 95.8%. Analysis condition B: Retention time=1.78 min; ESI-MS(+) m/z [M+2H]²⁺: 1111.2.

### Preparation of Example 1218

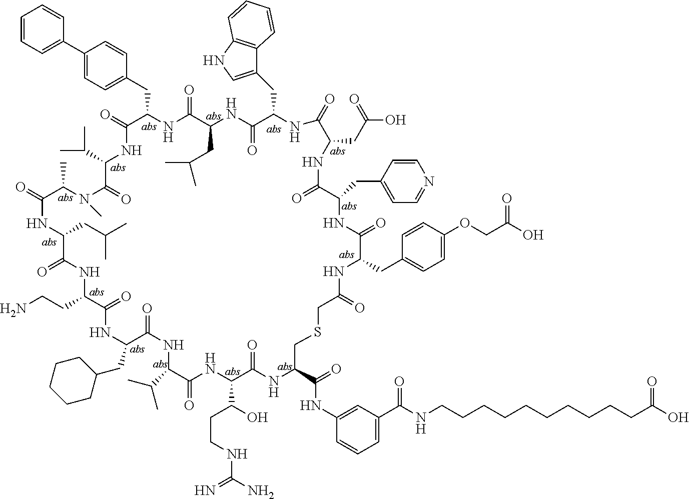 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](C(O)CCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1218 was prepared on a 50 μmol scale. The yield of the product was 3.6 mg, and its estimated purity by LCMS analysis was 97.5%. Analysis condition B: Retention time=1.73 min; ESI-MS(+) m/z [M+2H]²⁺: 1139.1.

### Preparation of Example 1219

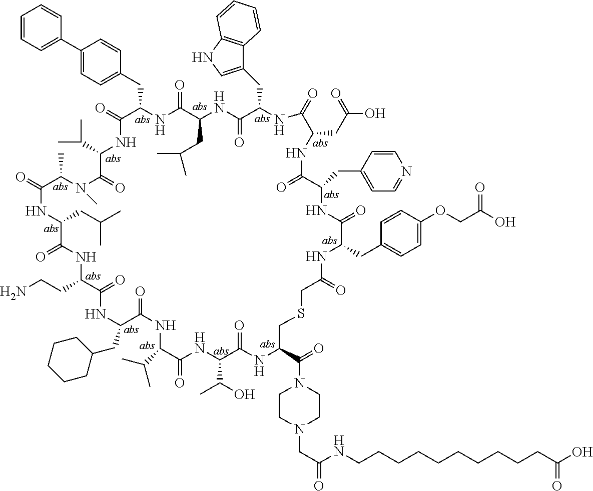 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCN(CC(=O)NCCCCCCCCCCC(=O)O)CC2)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1219 was prepared on a 50 μmol scale. The yield of the product was 14.5 mg, and its estimated purity by LCMS analysis was 85.2%. Analysis condition A: Retention time=1.84 min; ESI-MS(+) m/z [M+2H]²⁺: 1115.1.

### Preparation of Example 1220

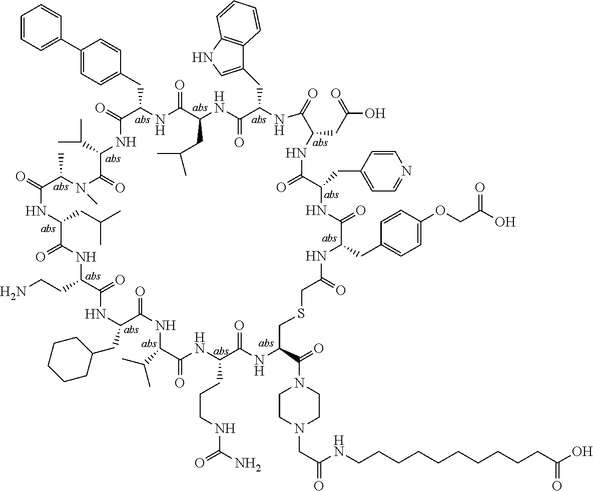 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCN(CC(=O)NCCCCCCCCCCC(=O)O)CC2)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1220 was prepared on a 50 μmol scale. The yield of the product was 3.7 mg, and its estimated purity by LCMS analysis was 97.8%. Analysis condition A: Retention time=1.77 min; ESI-MS(+) m/z [M+2H]²⁺: 1143.2.

### Preparation of Example 1221

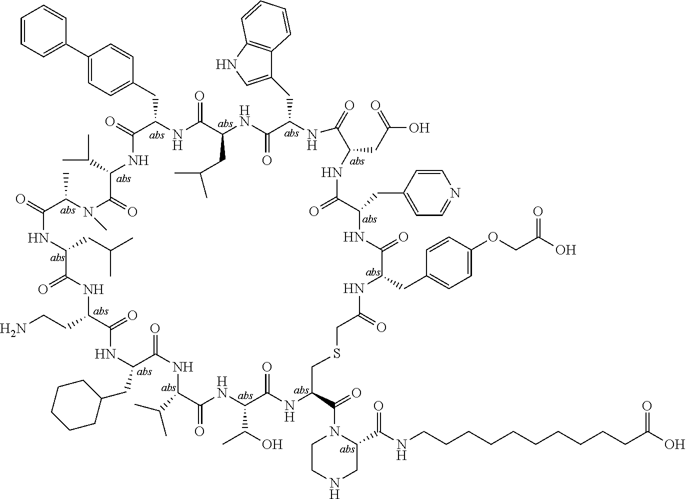 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCNC[C@H]2C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1221 was prepared on a 50 μmol scale. The yield of the product was 15.6 mg, and its estimated purity by LCMS analysis was 93.4%. Analysis condition A: Retention time=1.79 min; ESI-MS(+) m/z [M+2H]²⁺: 1108.1.

### Preparation of Example 1222

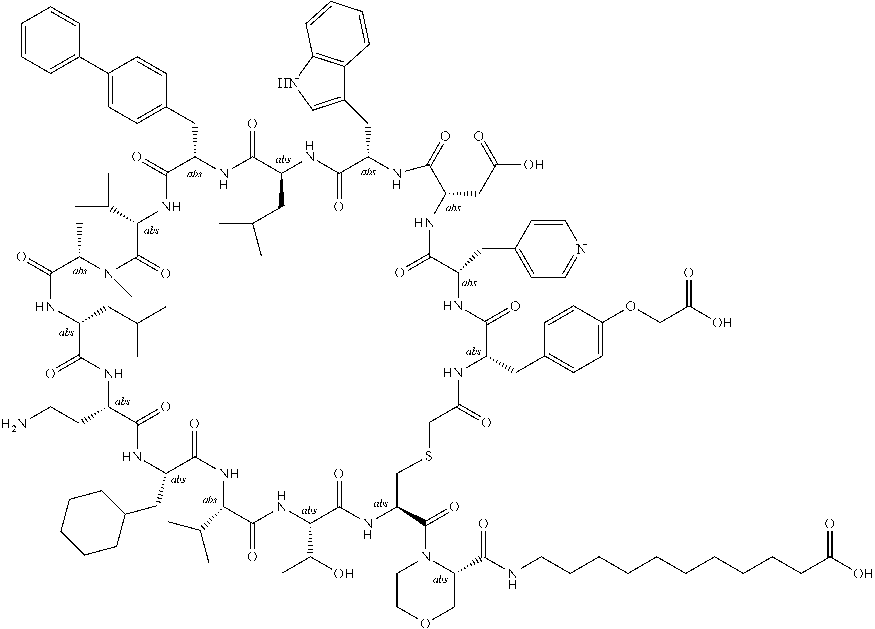 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCOC[C@H]2C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1222 was prepared on a 50 μmol scale. The yield of the product was 12.5 mg, and its estimated purity by LCMS analysis was 95.8%. Analysis condition A: Retention time=1.78 min; ESI-MS(+) m/z [M+2H]²⁺: 1108.

### Preparation of Example 1223

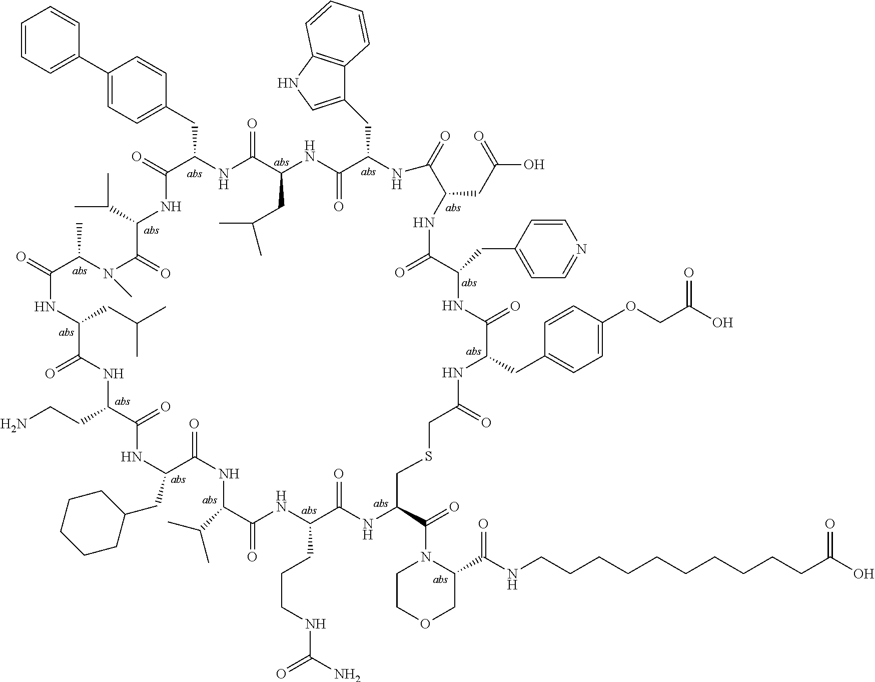 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCOC[C@H]2C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1223 was prepared on a 50 μmol scale. The yield of the product was 1.4 mg, and its estimated purity by LCMS analysis was 85.2%. Analysis condition A: Retention time=1.73 min; ESI-MS(+) m/z [M+2H]²⁺: 1137.2.

### Preparation of Example 1224

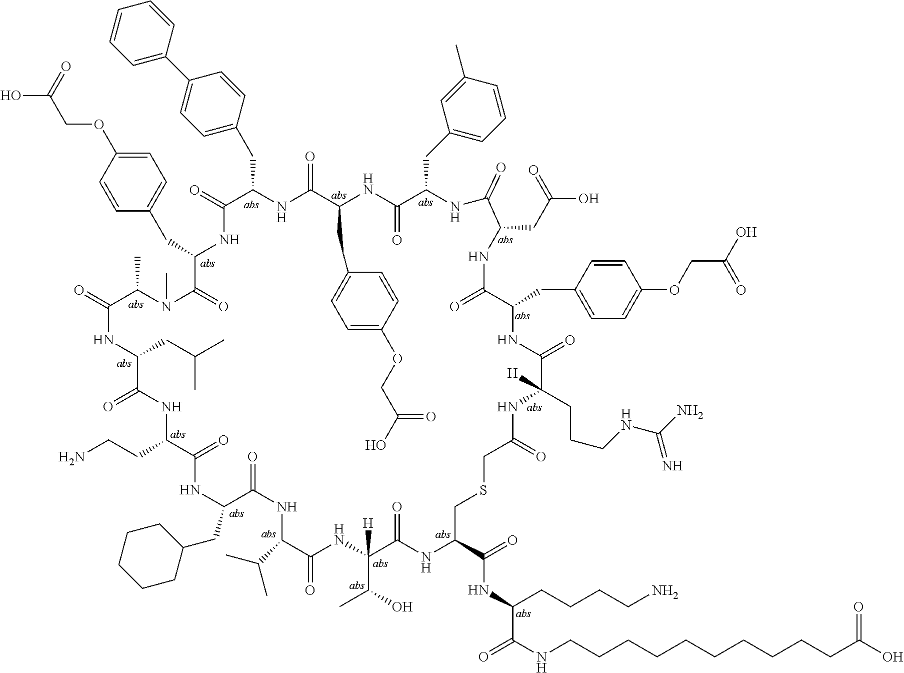 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1224 was prepared on a 50 μmol scale. The yield of the product was 2.6 mg, and its estimated purity by LCMS analysis was 96.7%. Analysis condition A: Retention time=1.44 min; ESI-MS(+) m/z [M+3H]³⁺: 815.9.

### Preparation of Example 1225

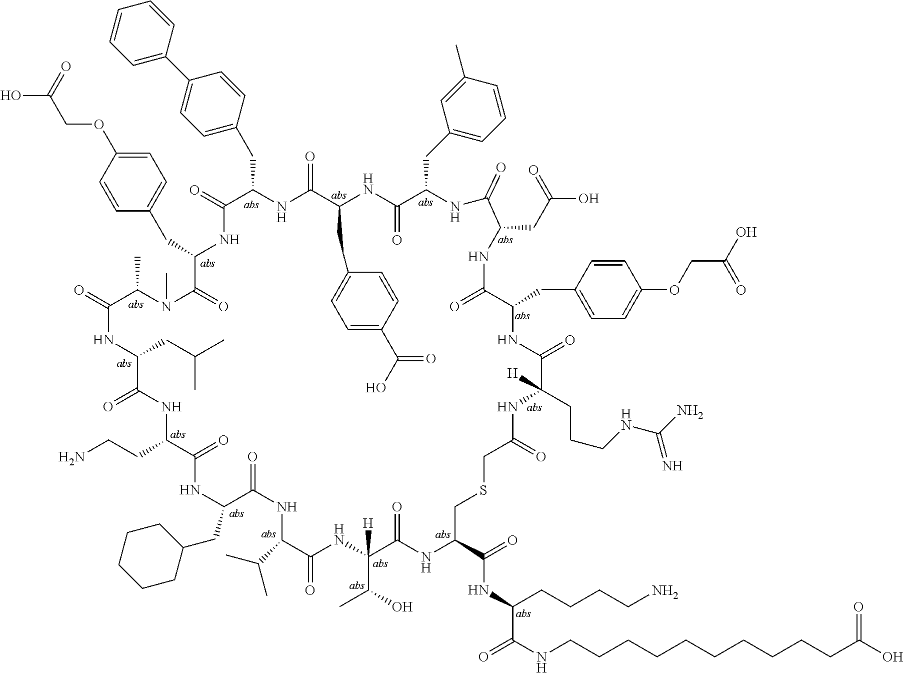 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1225 was prepared on a 50 μmol scale. The yield of the product was 21.1 mg, and its estimated purity by LCMS analysis was 98.4%. Analysis condition B: Retention time=1.71 min; ESI-MS(+) m/z [M+3H]³⁺: 805.

### Preparation of Example 1226

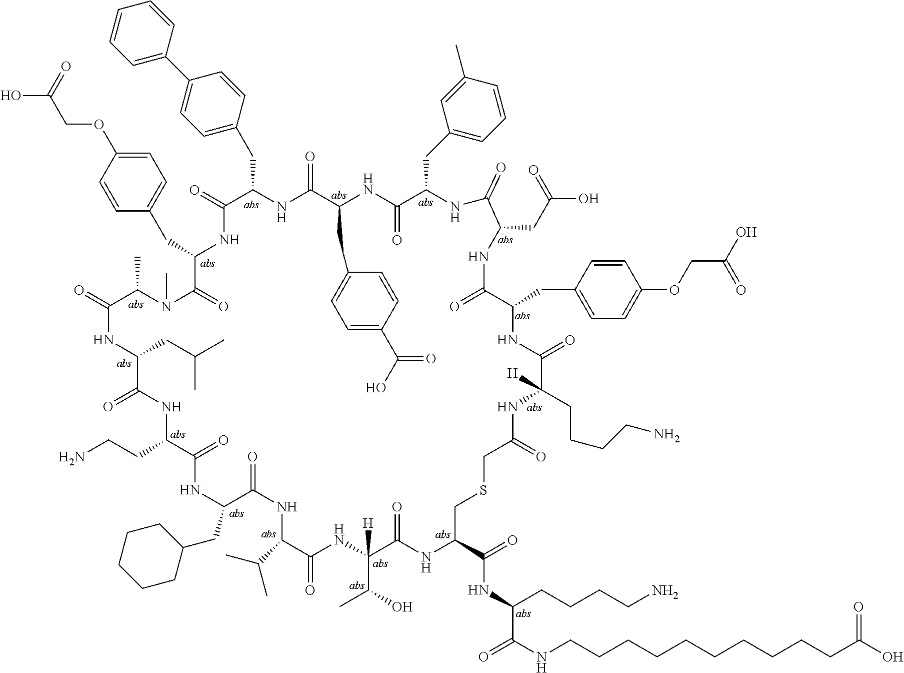 [SMILES: Cc1cccc(C[C@@H]2NC(=O)[C@H](CC(=O)O)NC(=O)[C@H](Cc3ccc(OCC(=O)O)cc3)NC(=O)[C@H](CCCCN)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1226 was prepared on a 50 μmol scale. The yield of the product was 5 mg, and its estimated purity by LCMS analysis was 91.7%. Analysis condition B: Retention time=1.61 min; ESI-MS(+) m/z [M+3H]³⁺: 796.1.

### Preparation of Example 1227

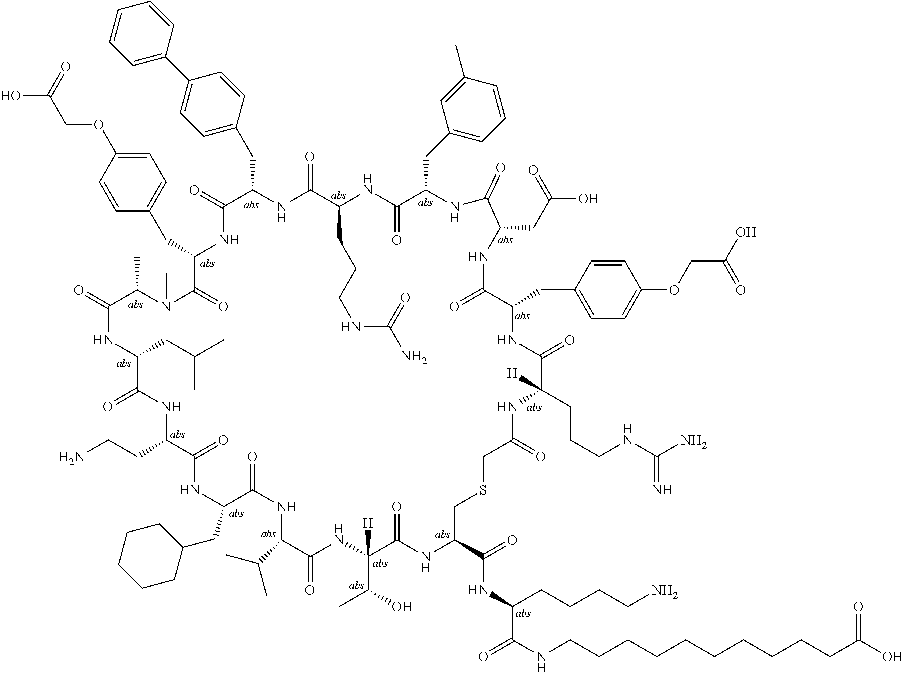 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCNC(N)=O)NC2=O)c1]

Example 1227 was prepared on a 50 μmol scale. The yield of the product was 14.2 mg, and its estimated purity by LCMS analysis was 91.3%. Analysis condition B: Retention time=1.63 min; ESI-MS(+) m/z [M+3H]³⁺: 794.3.

### Preparation of Example 1228

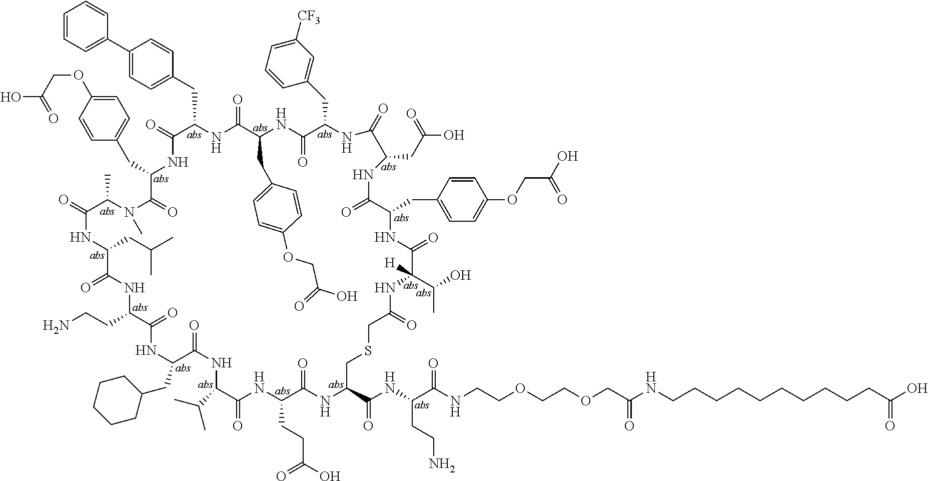 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1228 was prepared on a 50 μmol scale. The yield of the product was 30.9 mg, and its estimated purity by LCMS analysis was 95.6%. Analysis condition A: Retention time=1.92 min; ESI-MS(+) m/z [M+2H]²⁺: 1294.2.

### Preparation of Example 1229

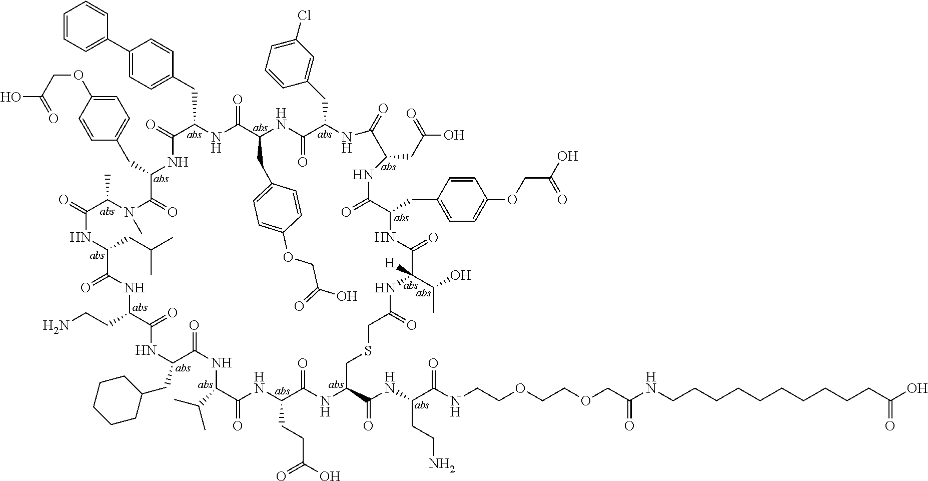 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(Cl)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1229 was prepared on a 50 μmol scale. The yield of the product was 6.8 mg, and its estimated purity by LCMS analysis was 100%. Analysis condition A: Retention time=1.37 min; ESI-MS(+) m/z [M+2H]²⁺: 1278.1.

### Preparation of Example 1230

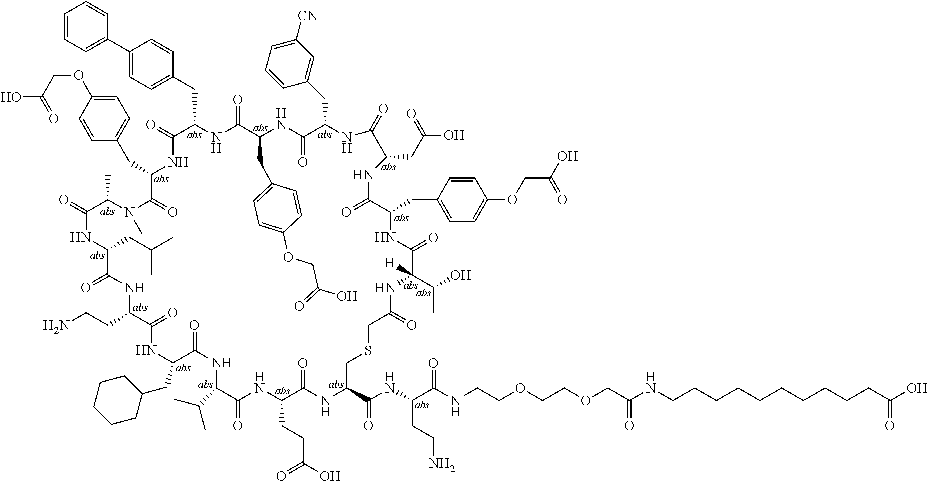 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C#N)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1230 was prepared on a 50 μmol scale. The yield of the product was 1.9 mg, and its estimated purity by LCMS analysis was 82.4%. Analysis condition A: Retention time=1.35 min; ESI-MS(+) m/z [M+2H]²⁺: 1273.1.

### Preparation of Example 1231

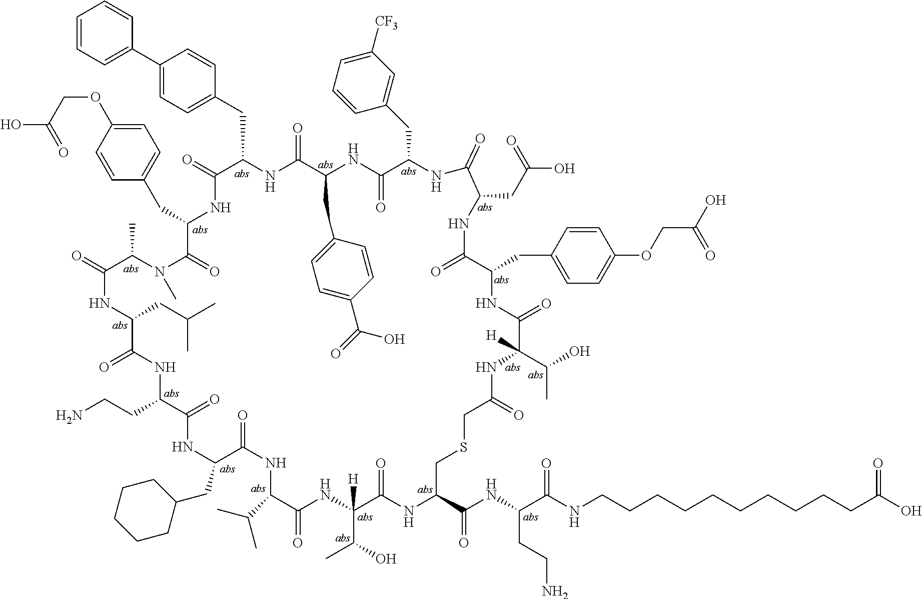 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1231 was prepared on a 50 μmol scale. The yield of the product was 1.9 mg, and its estimated purity by LCMS analysis was 100%. Analysis condition A: Retention time=1.56 min; ESI-MS(+) m/z [M+2H]²⁺: 1193.1.

### Preparation of Example 1232

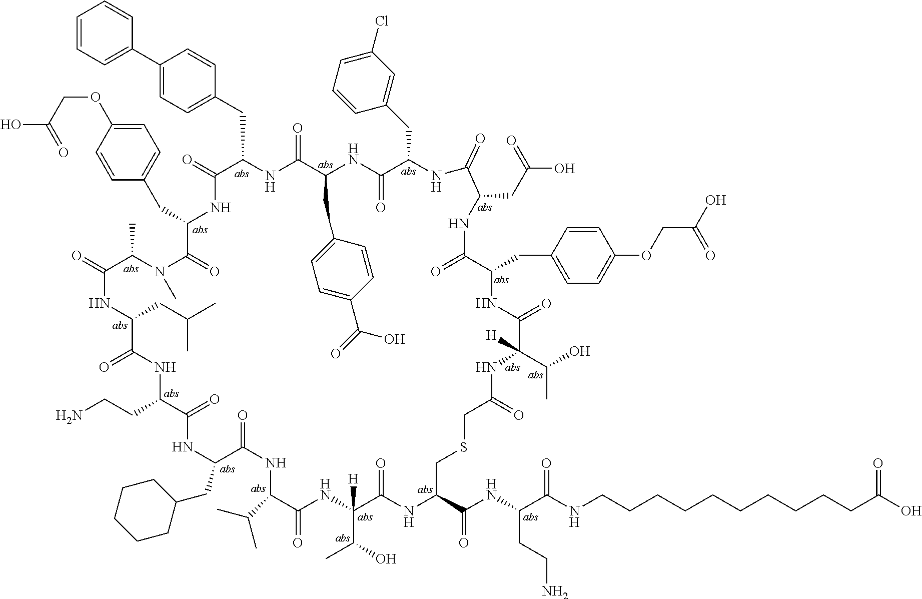 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(Cl)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1232 was prepared on a 50 μmol scale. The yield of the product was 24.2 mg, and its estimated purity by LCMS analysis was 100%. Analysis condition A: Retention time=1.48 min; ESI-MS(+) m/z [M+2H]²⁺: 1176.

### Preparation of Example 1233

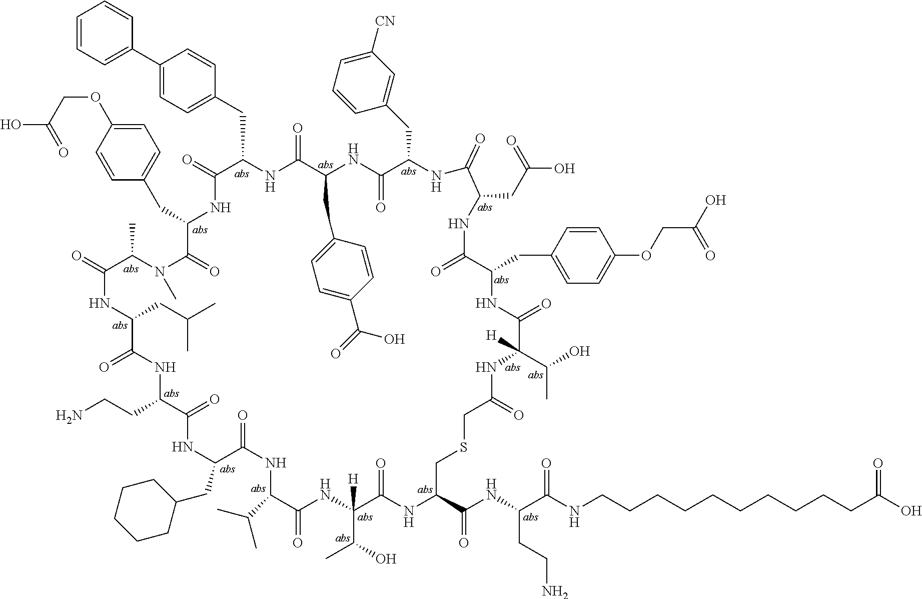 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C#N)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1233 was prepared on a 50 μmol scale. The yield of the product was 25.6 mg, and its estimated purity by LCMS analysis was 98.5%. Analysis condition A: Retention time=1.42 min; ESI-MS(+) m/z [M+2H]²⁺: 1171.

### Preparation of Example 1234

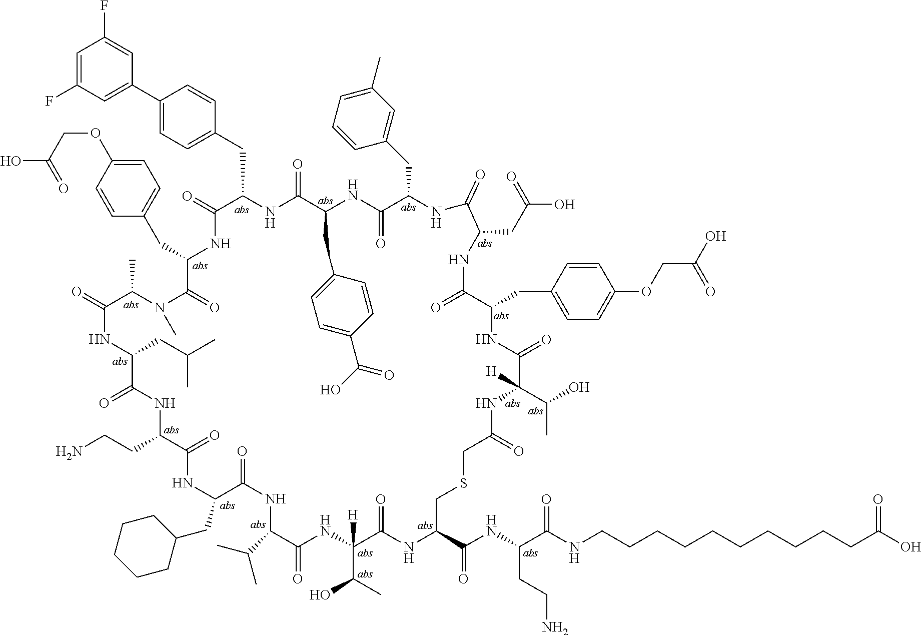 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4cc(F)cc(F)c4)cc3)NC(=O)[C@H](Cc3ccc(C(=O)O)cc3)NC2=O)c1]

Example 1234 was prepared on a 50 μmol scale. The yield of the product was 12.9 mg, and its estimated purity by LCMS analysis was 89%. Analysis condition A: Retention time=1.48 min; ESI-MS(+) m/z [M+2H]²⁺: 1184.1.

### Preparation of Example 1235

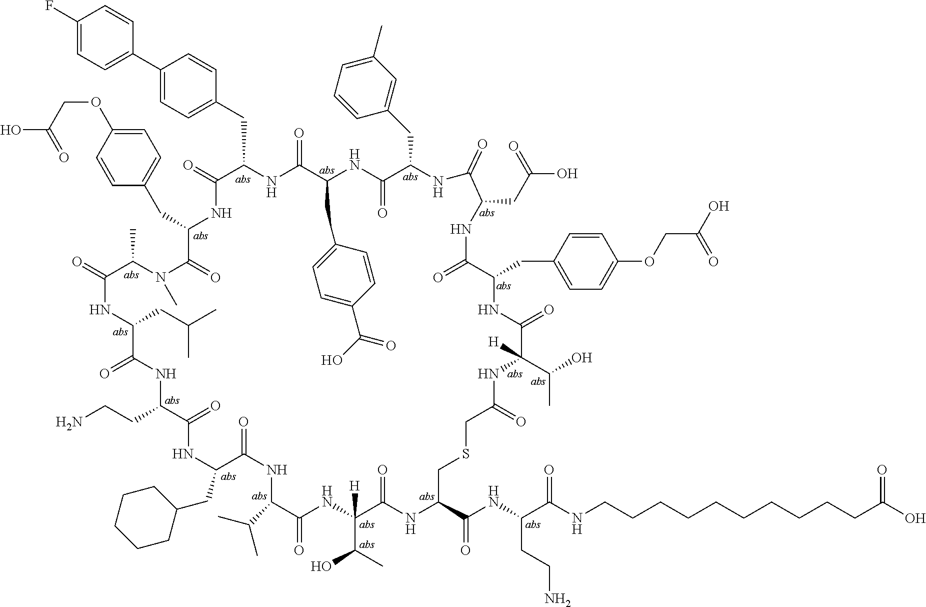 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccc(F)cc4)cc3)NC(=O)[C@H](Cc3ccc(C(=O)O)cc3)NC2=O)c1]

Example 1235 was prepared on a 50 μmol scale. The yield of the product was 17.4 mg, and its estimated purity by LCMS analysis was 94.6%. Analysis condition A: Retention time=1.64 min; ESI-MS(+) m/z [M+2H]²⁺: 1175.3.

### Preparation of Example 1236

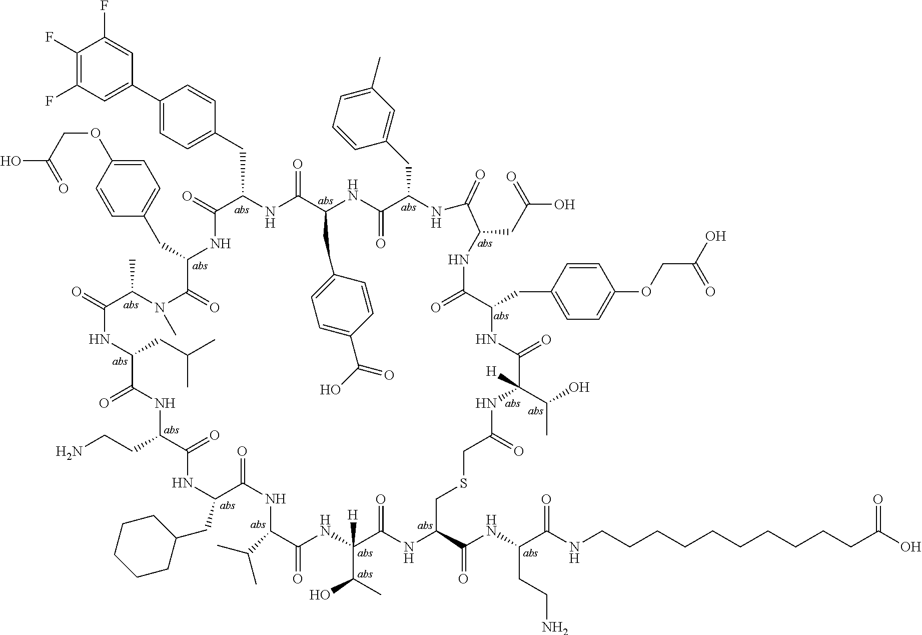 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4cc(F)c(F)c(F)c4)cc3)NC(=O)[C@H](Cc3ccc(C(=O)O)cc3)NC2=O)c1]

Example 1236 was prepared on a 50 μmol scale. The yield of the product was 33.3 mg, and its estimated purity by LCMS analysis was 91.5%. Analysis condition A: Retention time=1.51 min; ESI-MS(+) m/z [M+2H]²⁺: 1193.1.

### Preparation of Example 1237

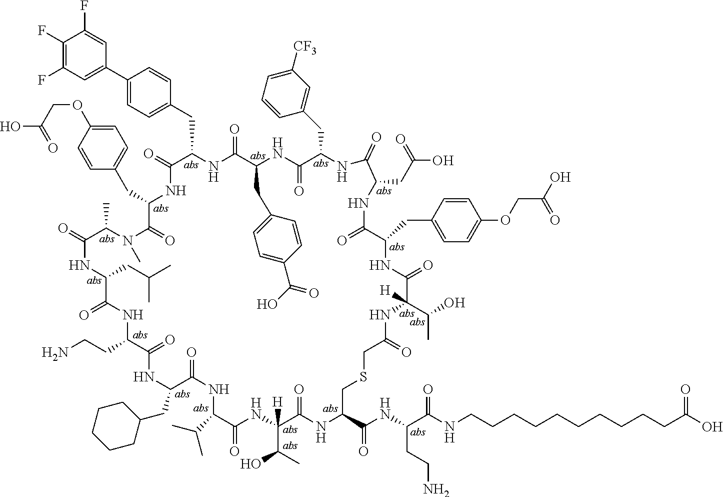 [SMILES: CC(C)C[C@H]1NC(=O)[C@H](C)N(C)C(=O)[C@H](Cc2ccc(OCC(=O)O)cc2)NC(=O)[C@H](Cc2ccc(-c3cc(F)c(F)c(F)c3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1237 was prepared on a 50 μmol scale. The yield of the product was 57.2 mg, and its estimated purity by LCMS analysis was 90.7%. Analysis condition B: Retention time=1.97 min; ESI-MS(+) m/z [M+2H]²⁺: 1220.2.

### Preparation of Example 1238

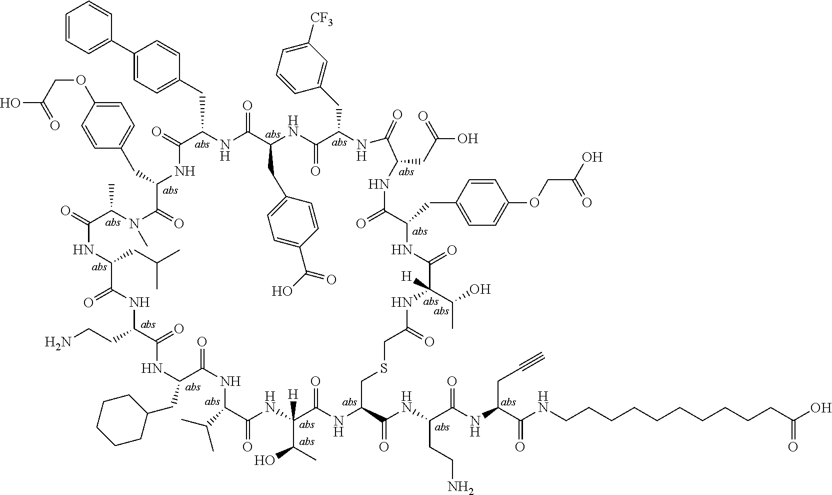 [SMILES: C#CC[C@H](NC(=O)[C@H](CCN)NC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2cccc(C(F)(F)F)c2)C(=O)N[C@@H](Cc2ccc(C(=O)O)cc2)C(=O)N[C@@H](Cc2ccc(-c3ccccc3)cc2)C(=O)N[C@@H](Cc2ccc(OCC(=O)O)cc2)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)NCCCCCCCCCCC(=O)O]

Example 1238 was prepared on a 50 μmol scale. The yield of the product was 26.3 mg, and its estimated purity by LCMS analysis was 91.1%. Analysis condition A: Retention time=1.75 min; ESI-MS(+) m/z [M+2H]²⁺: 1241.2.

### Preparation of Example 1239

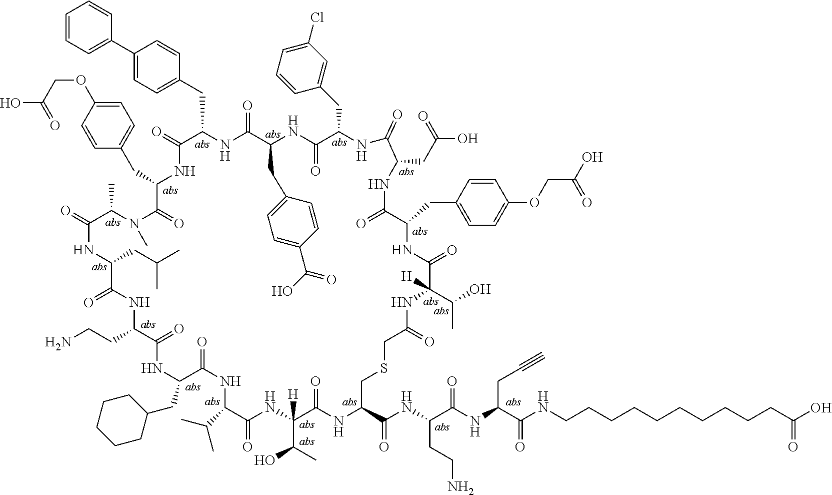 [SMILES: C#CC[C@H](NC(=O)[C@H](CCN)NC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2cccc(Cl)c2)C(=O)N[C@@H](Cc2ccc(C(=O)O)cc2)C(=O)N[C@@H](Cc2ccc(-c3ccccc3)cc2)C(=O)N[C@@H](Cc2ccc(OCC(=O)O)cc2)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)NCCCCCCCCCCC(=O)O]

Example 1239 was prepared on a 50 μmol scale. The yield of the product was 19.1 mg, and its estimated purity by LCMS analysis was 91.4%. Analysis condition A: Retention time=1.65, 1.7 min; ESI-MS(+) m/z [M+2H]²⁺: 1224.

### Preparation of Example 1240

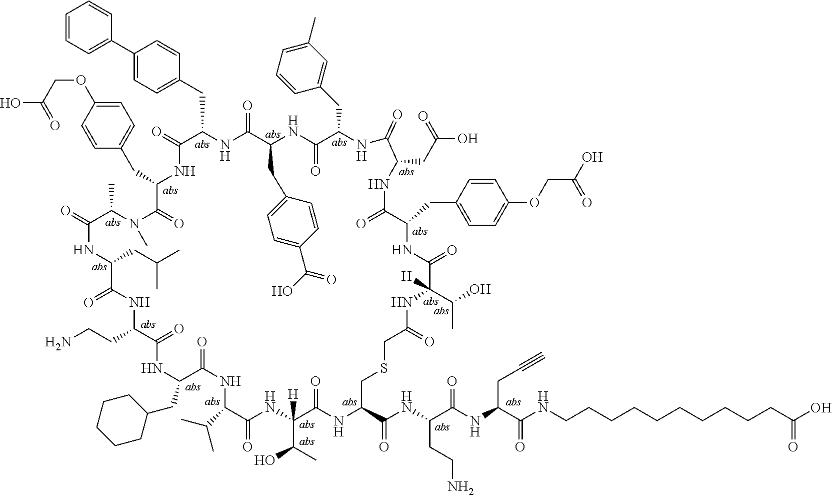 [SMILES: C#CC[C@H](NC(=O)[C@H](CCN)NC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(OCC(=O)O)cc2)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)NCCCCCCCCCCC(=O)O]

Example 1240 was prepared on a 50 μmol scale. The yield of the product was 16.9 mg, and its estimated purity by LCMS analysis was 98.4%. Analysis condition B: Retention time=1.93 min; ESI-MS(+) m/z [M+2H]²⁺: 1213.9.

### Preparation of Example 1241

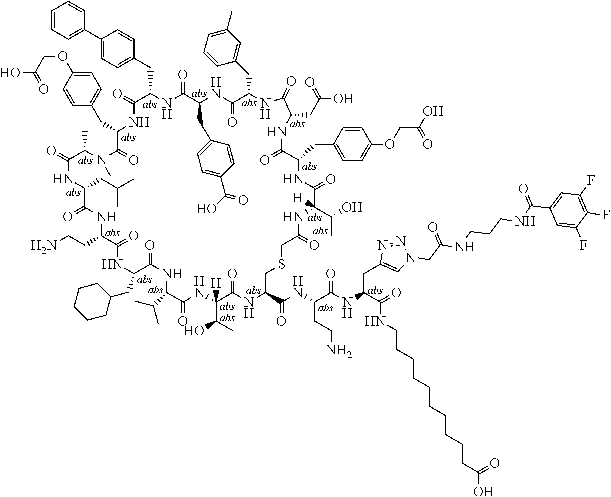 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)N[C@@H](Cc3cn(CC(=O)NCCCNC(=O)c4cc(F)c(F)c(F)c4)nn3)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1241 was prepared on a 31 μmol scale. The yield of the product was 11.7 mg, and its estimated purity by LCMS analysis was 99.3%. Analysis condition B: Retention time=2.04 min; ESI-MS(+) m/z [M+2H]²⁺: 1371.1.

### Preparation of Example 1242

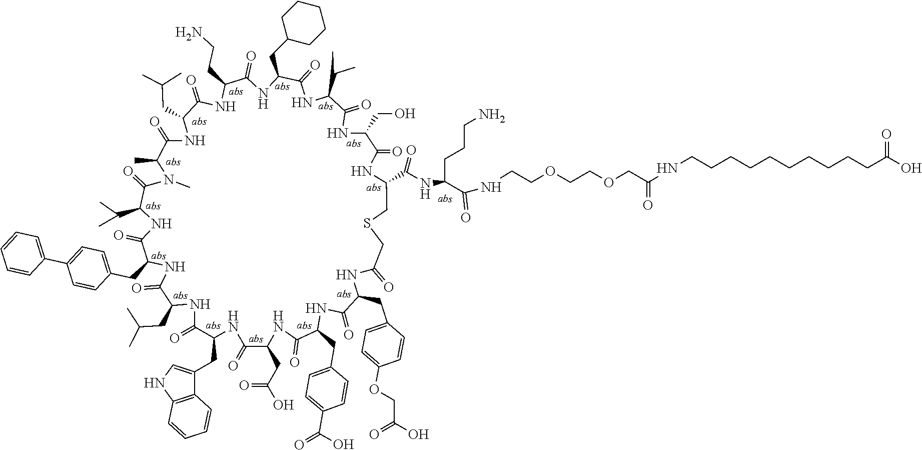 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1242 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.7 mg, and its estimated purity by LCMS analysis was 97.1%. Analysis condition A: Retention time=1.63 min; ESI-MS(+) m/z [M+2H]²⁺: 1196.

### Preparation of Example 1243

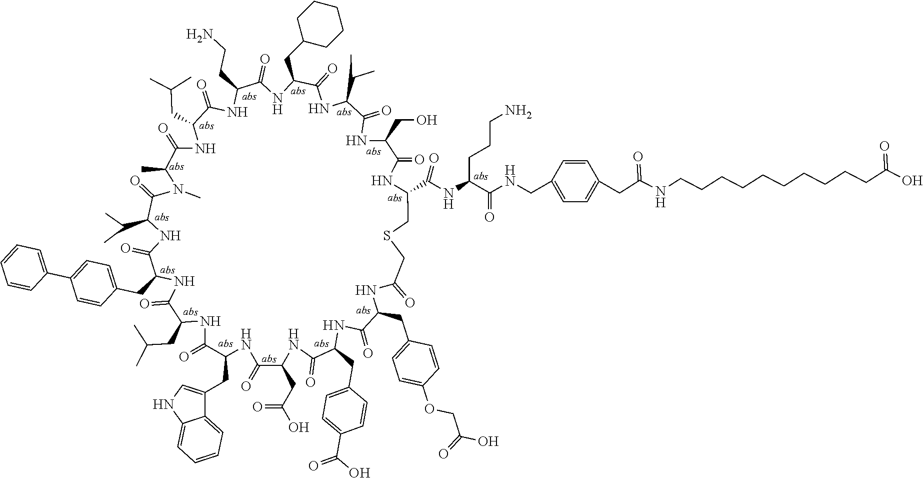 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCc2ccc(CC(=O)NCCCCCCCCCCC(=O)O)cc2)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1243 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 2-(4-(((((9H-fluoren-9-yl)methoxy)carbonyl)amino)methyl)phenyl)acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 20% B, 20-60% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 31 mg, and its estimated purity by LCMS analysis was 97.6%. Analysis condition A: Retention time=1.47 min; ESI-MS(+) m/z [M+2H]²⁺: 1197.1.

### Preparation of Example 1244

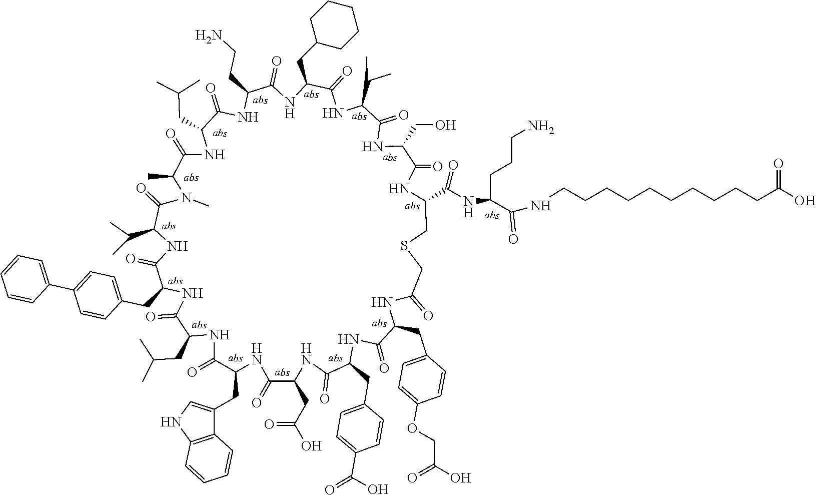 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1244 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.4 mg, and its estimated purity by LCMS analysis was 95.6%. Analysis condition A: Retention time=1.65 min; ESI-MS(+) m/z [M+2H]²⁺: 1117.

### Preparation of Example 1245

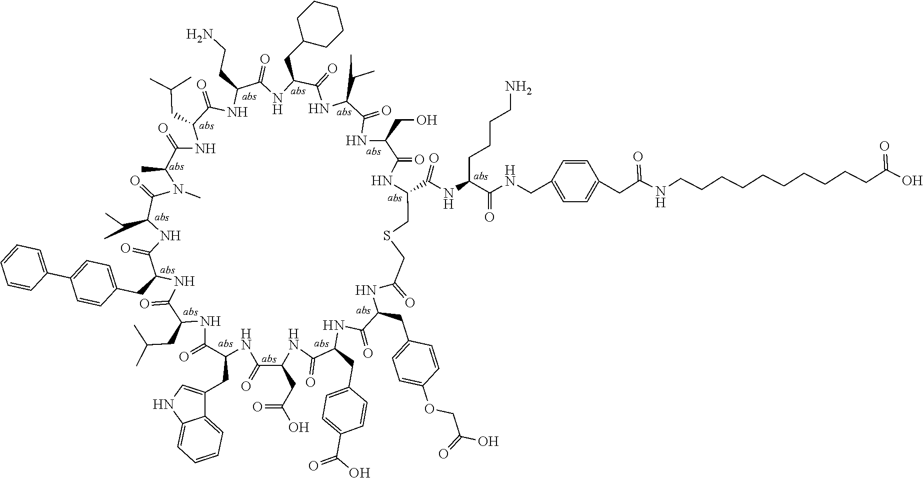 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCc2ccc(CC(=O)NCCCCCCCCCCC(=O)O)cc2)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1245 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 2-(4-(((((9H-fluoren-9-yl)methoxy)carbonyl)amino)methyl)phenyl)acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14.7 mg, and its estimated purity by LCMS analysis was 92.4%. Analysis condition A: Retention time=1.7 min; ESI-MS(+) m/z [M+2H]²⁺: 1204.1.

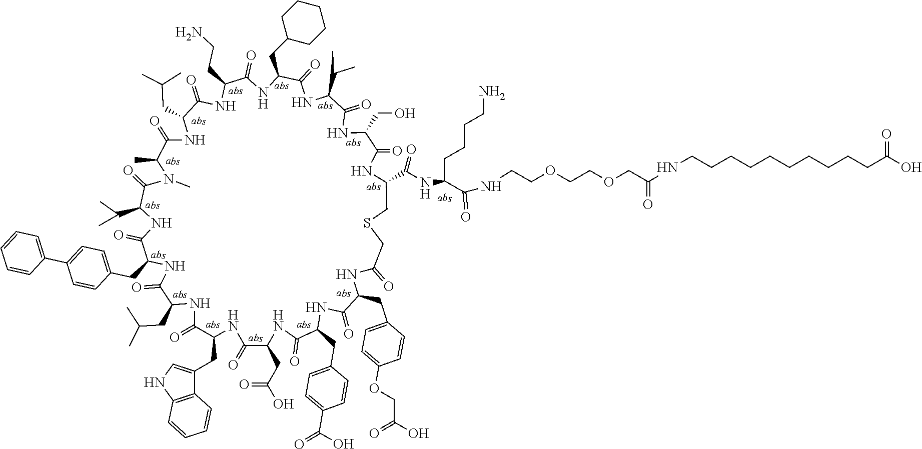 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1246 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 19.2 mg, and its estimated purity by LCMS analysis was 96.6%. Analysis condition B: Retention time=1.84 min; ESI-MS(+) m/z [M+2H]²⁺: 1203.2.

### Preparation of Example 1247

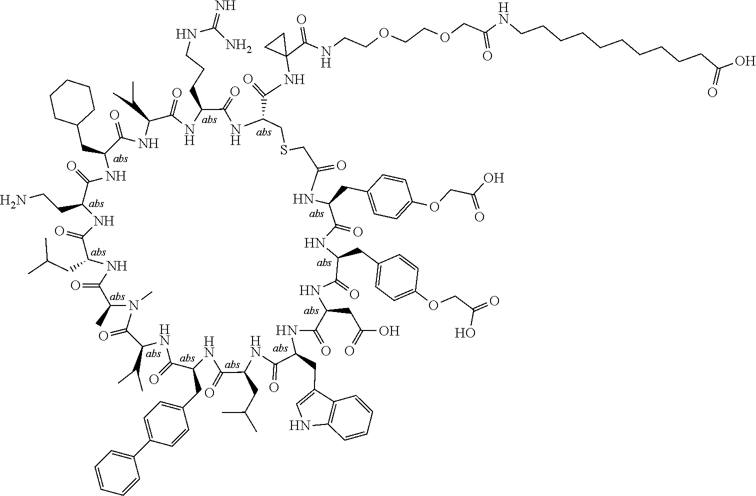 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NC2(C(=O)NCCOCCOCC(=O)NCCCCCCCCCCC(=O)O)CC2)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1247 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.1 mg, and its estimated purity by LCMS analysis was 97%. Analysis condition A: Retention time=1.71 min; ESI-MS(+) m/z [M+2H]²⁺: 1230.2.

### Preparation of Example 1248

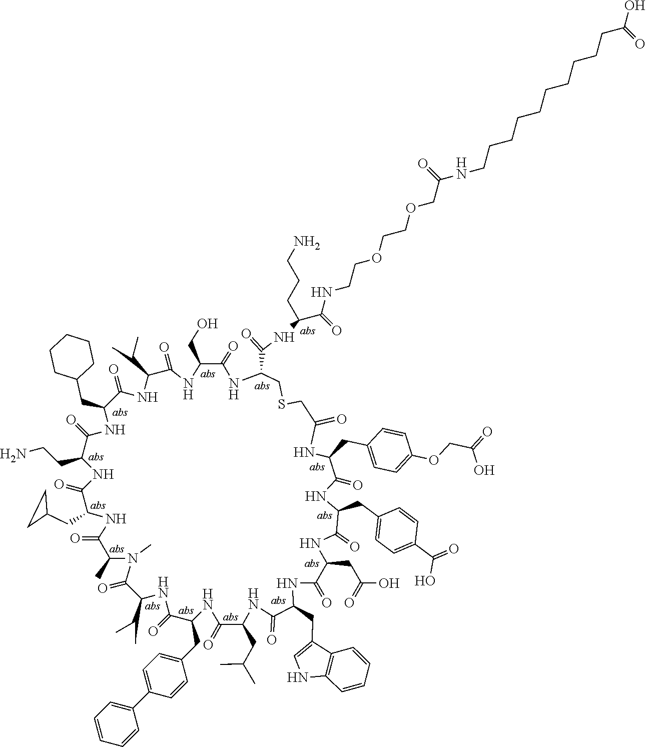 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC2CC2)NC(=O)[C@H](C)N(C)C(=O)[C@H](C(C)C)NC(=O)[C@H](Cc2ccc(-c3ccccc3)cc2)NC1=O]

Example 1248 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-D-Ala(cyclopropyl); “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.5 mg, and its estimated purity by LCMS analysis was 100%. Analysis condition A: Retention time=1.53 min; ESI-MS(+) m/z [M+2H]²⁺: 1195.1.

### Preparation of Example 1249

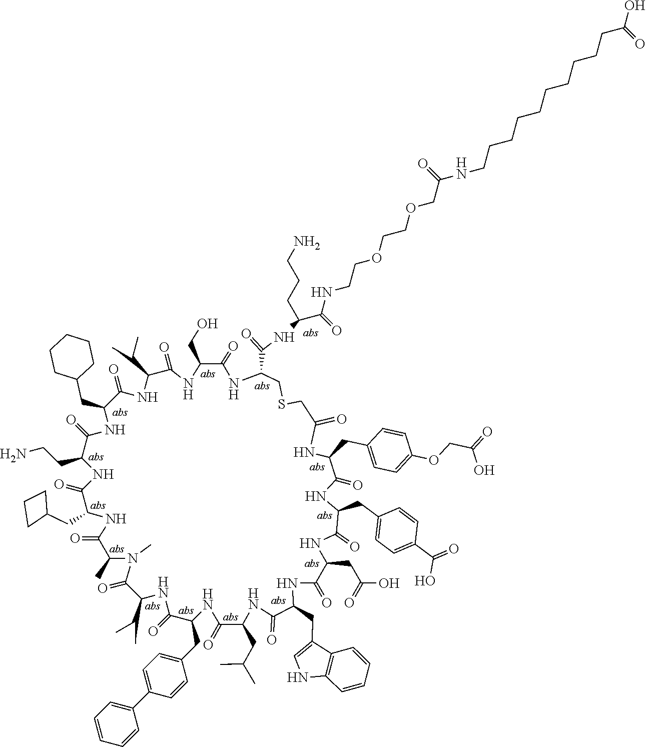 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC2CCC2)NC(=O)[C@H](C)N(C)C(=O)[C@H](C(C)C)NC(=O)[C@H](Cc2ccc(-c3ccccc3)cc2)NC1=O]

Example 1249 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-D-Ala(cyclobutyl), “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.3 mg, and its estimated purity by LCMS analysis was 90.1%. Analysis condition B: Retention time=1.86 min; ESI-MS(+) m/z [M+2H]²⁺: 1202.1.

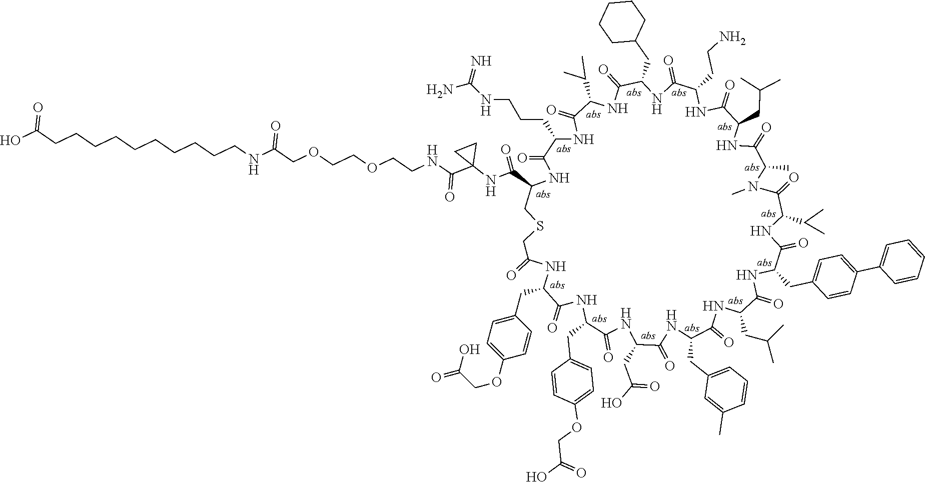 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NC3(C(=O)NCCOCCOCC(=O)NCCCCCCCCCCC(=O)O)CC3)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 1250 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 28% B, 28-68% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 44.4 mg, and its estimated purity by LCMS analysis was 95.5%. Analysis condition B: Retention time=1.91 min; ESI-MS(+) m/z [M+2H]²⁺: 1216.9.

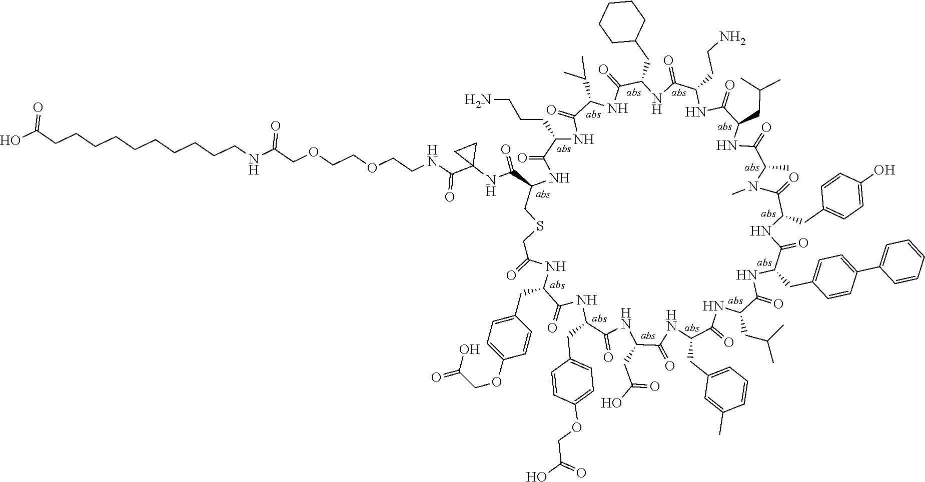 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NC3(C(=O)NCCOCCOCC(=O)NCCCCCCCCCCC(=O)O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CC(C)C)NC2=O)c1]

Example 1251 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 30% B, 30-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 83.3 mg, and its estimated purity by LCMS analysis was 99.2%. Analysis condition B: Retention time=1.9 min; ESI-MS(+) m/z [M+2H]²⁺: 1228.2.

### Preparation of Example 1252

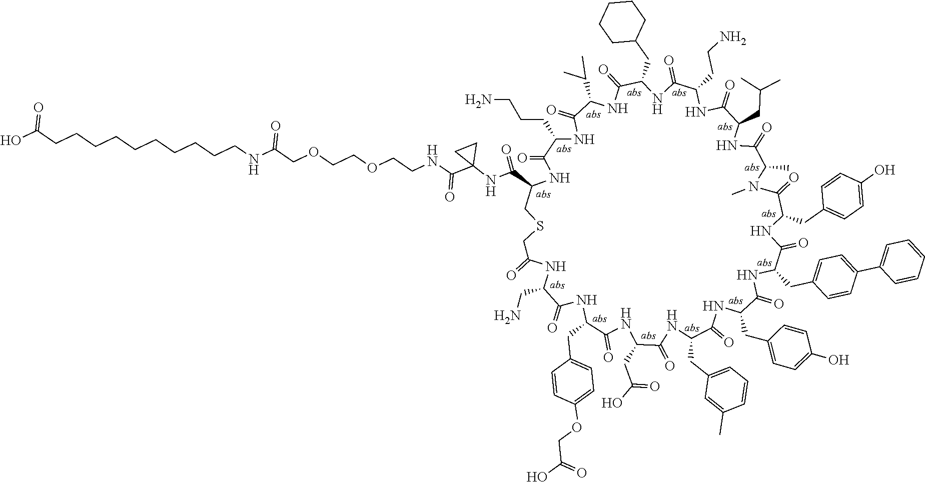 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(=O)NCCOCCOCC(=O)NCCCCCCCCCCC(=O)O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1252 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 39.4 mg, and its estimated purity by LCMS analysis was 97.4%. Analysis condition A: Retention time=1.73 min; ESI-MS(+) m/z [M+2H]²⁺: 1185.9.

### Preparation of Example 1253

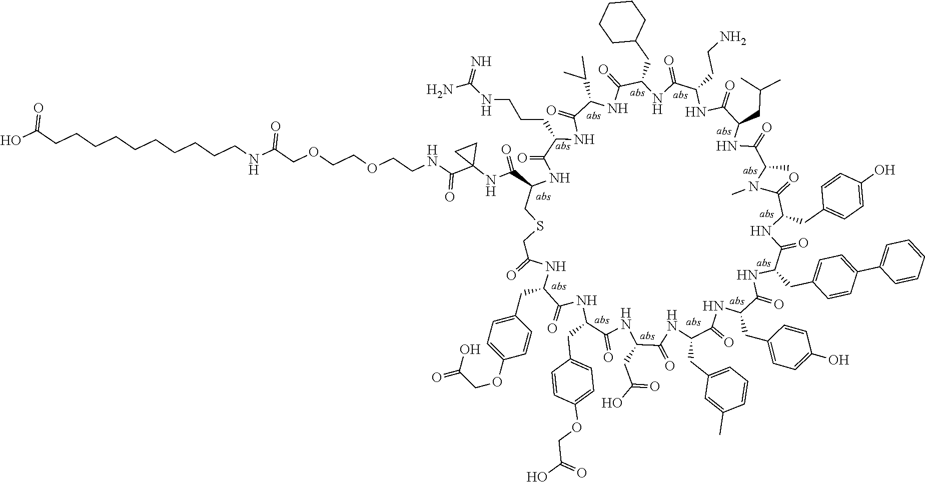 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NC3(C(=O)NCCOCCOCC(=O)NCCCCCCCCCCC(=O)O)CC3)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1253 was prepared, using 2-Chlorotrityl resin pre-loaded with 11-((((9H-fluoren-9-yl)methoxy)carbonyl)amino)undecanoic acid on a 50 □mol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 23% B, 23-63% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 92.1 mg, and its estimated purity by LCMS analysis was 97.1%. Analysis condition A: Retention time=1.54 min; ESI-MS(+) m/z [M+3H]³⁺: 850.2.

### Preparation of Example 1254

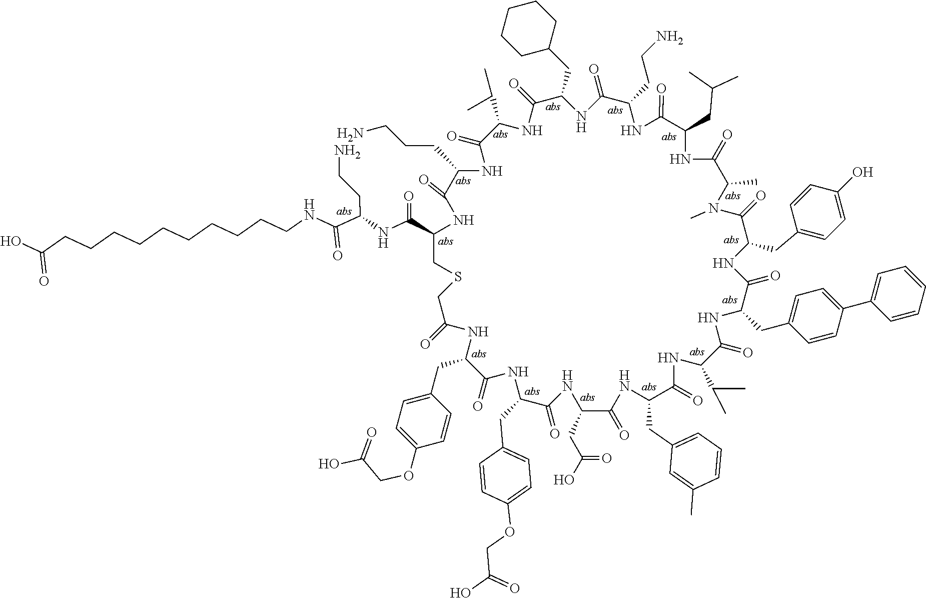 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1254 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.3 mg, and its estimated purity by LCMS analysis was 88.6%. Analysis condition B: Retention time=1.8 min; ESI-MS(+) m/z [M+2H]²⁺: 1157.3.

### Preparation of Example 1255

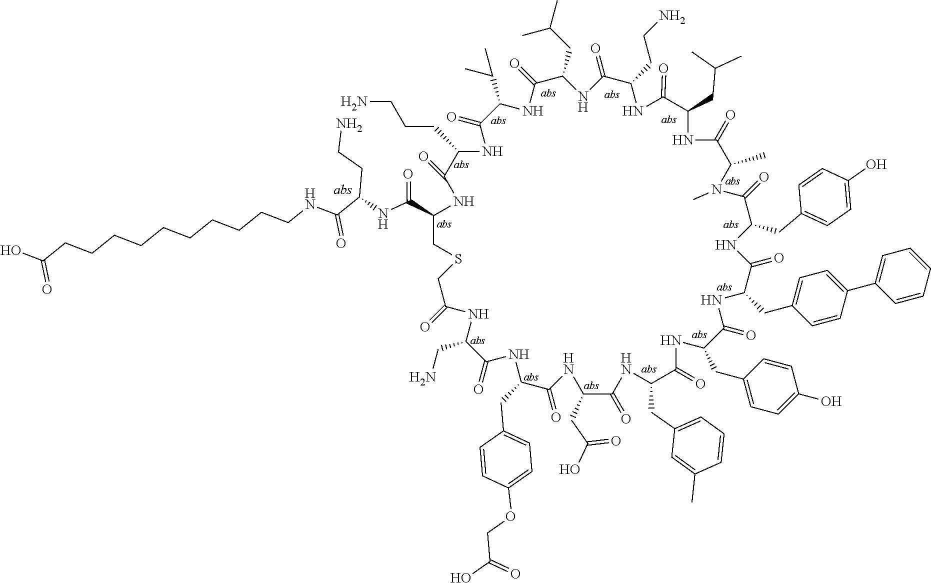 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1255 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 35.5 mg, and its estimated purity by LCMS analysis was 94.8%. Analysis condition A: Retention time=1.62 min; ESI-MS(+) m/z [M+2H]²⁺: 1101.9.

### Preparation of Example 1256

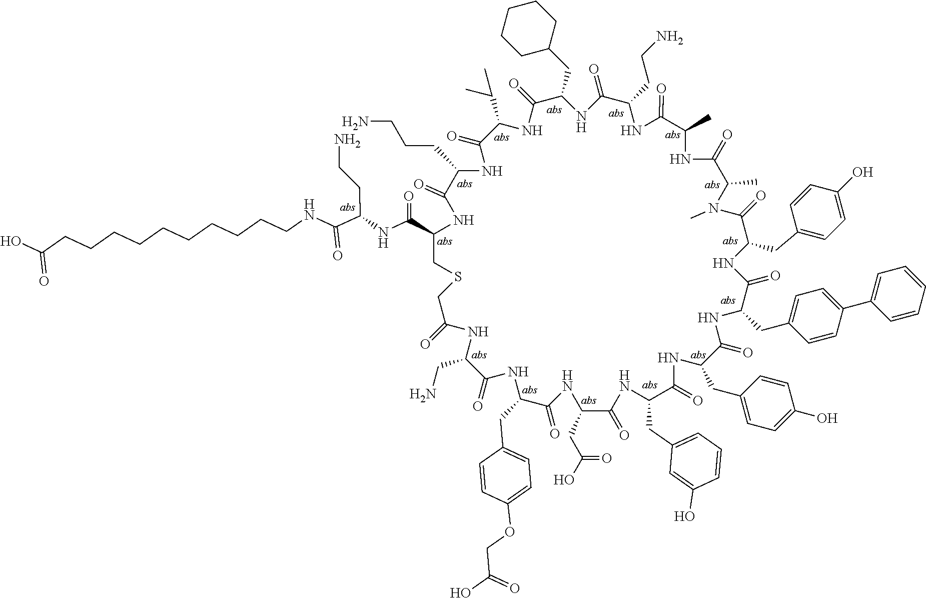 [SMILES: CC(C)[C@@H]1NC(=O)[C@H](CC2CCCCC2)NC(=O)[C@H](CCN)NC(=O)[C@@H](C)NC(=O)[C@H](C)N(C)C(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2cccc(O)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCN)NC1=O]

Example 1256 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 38.4 mg, and its estimated purity by LCMS analysis was 89.2%. Analysis condition A: Retention time=1.38 min; ESI-MS(+) m/z [M+2H]²⁺: 1102.2.

### Preparation of Example 1257

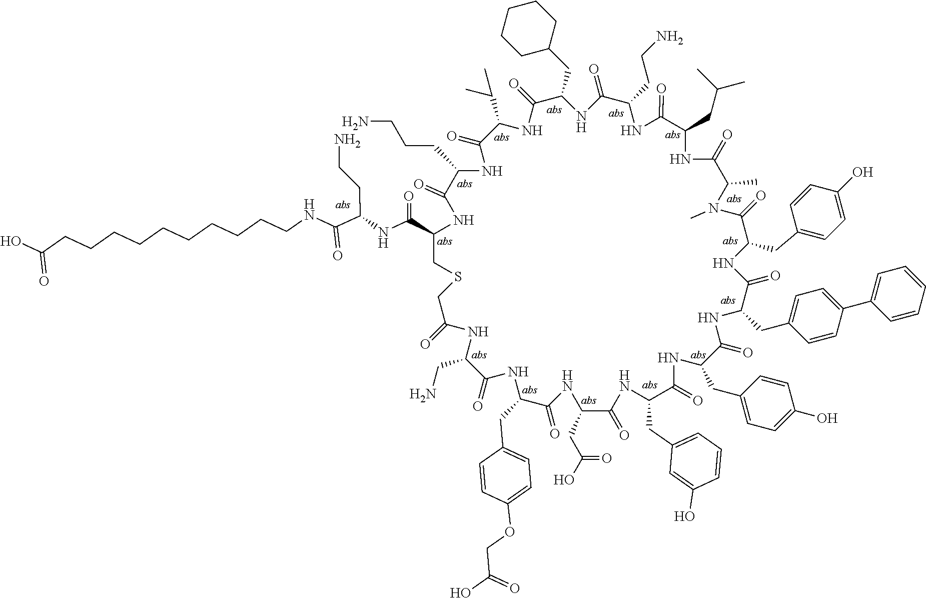 [SMILES: CC(C)C[C@H]1NC(=O)[C@H](C)N(C)C(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2cccc(O)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Example 1257 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 57.5 mg, and its estimated purity by LCMS analysis was 86%. Analysis condition A: Retention time=1.57 min; ESI-MS(+) m/z [M+2H]²⁺: 1123.2.

### Preparation of Example 1258

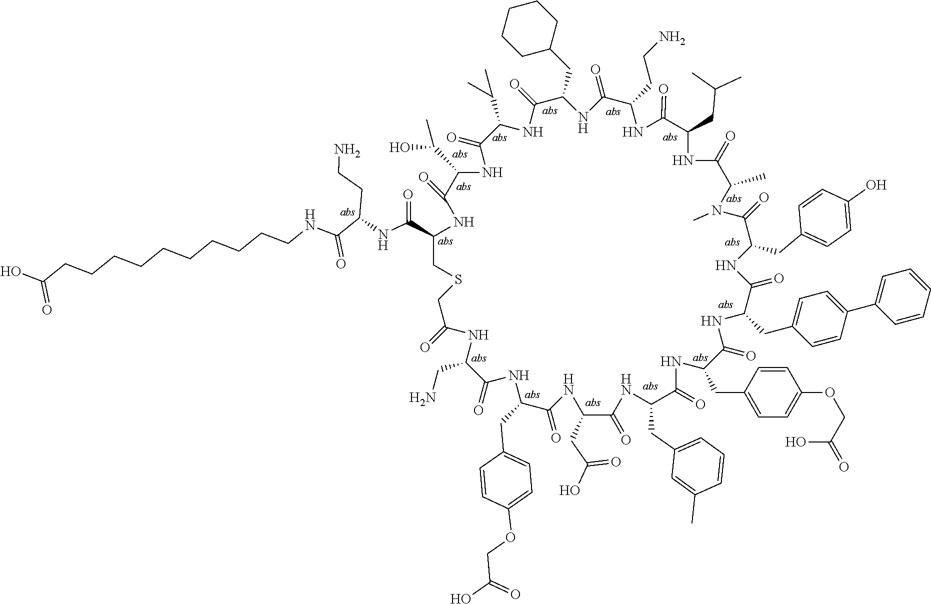 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1258 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.6 mg, and its estimated purity by LCMS analysis was 100%. Analysis condition A: Retention time=1.75 min; ESI-MS(+) m/z [M+2H]²⁺: 1144.1.

### Preparation of Example 1259

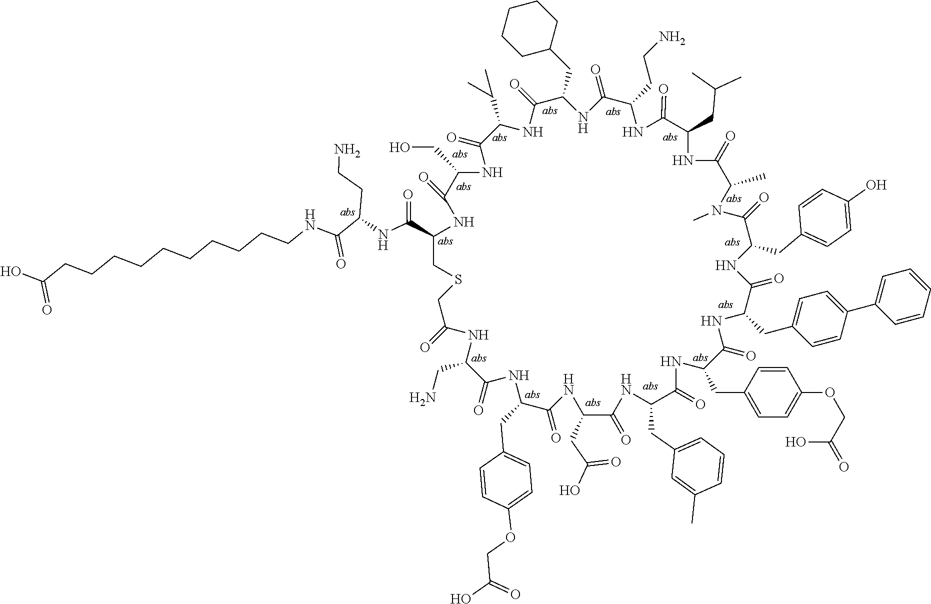 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1259 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.6 mg, and its estimated purity by LCMS analysis was 95.3%. Analysis condition B: Retention time=1.74 min; ESI-MS(+) m/z [M+2H]²⁺: 1137.2.

### Preparation of Example 1260

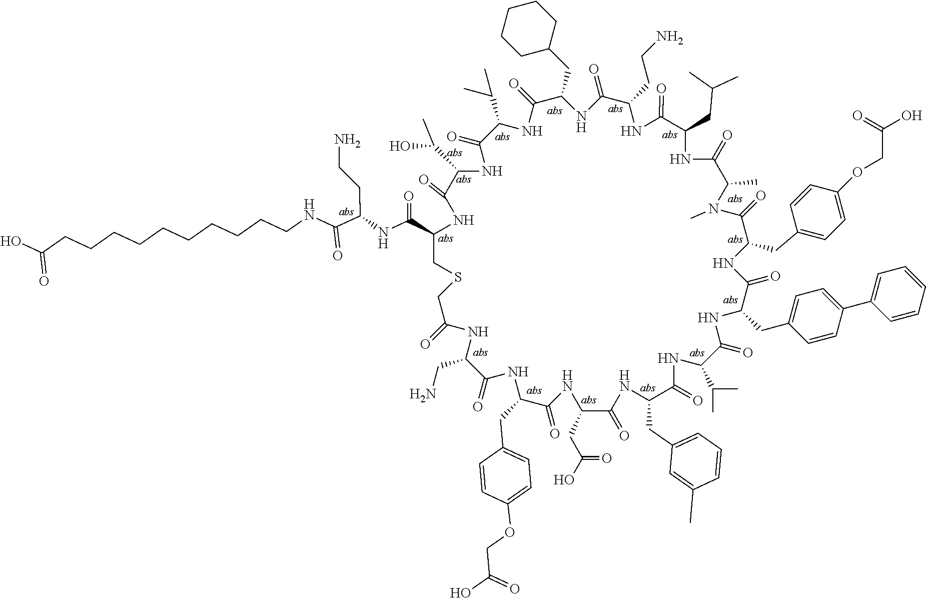 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 1260 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.6 mg, and its estimated purity by LCMS analysis was 95.9%. Analysis condition A: Retention time=1.52 min; ESI-MS(+) m/z [M+2H]²⁺: 1112.5.

### Preparation of Example 1261

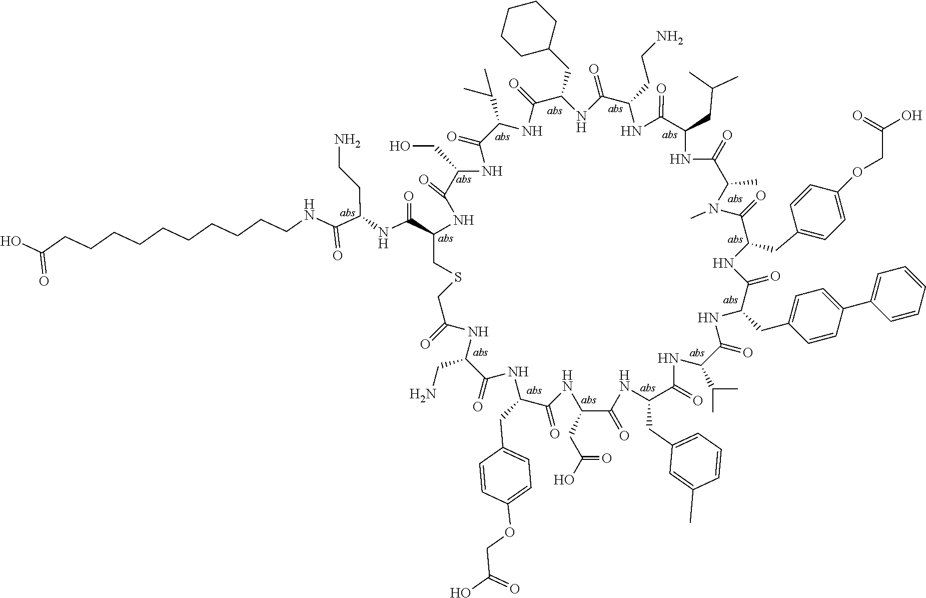 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 1261 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 20% B, 20-60% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 95.7%. Analysis condition A: Retention time=1.43 min; ESI-MS(+) m/z [M+2H]²⁺: 1105.4.

### Preparation of Example 1262

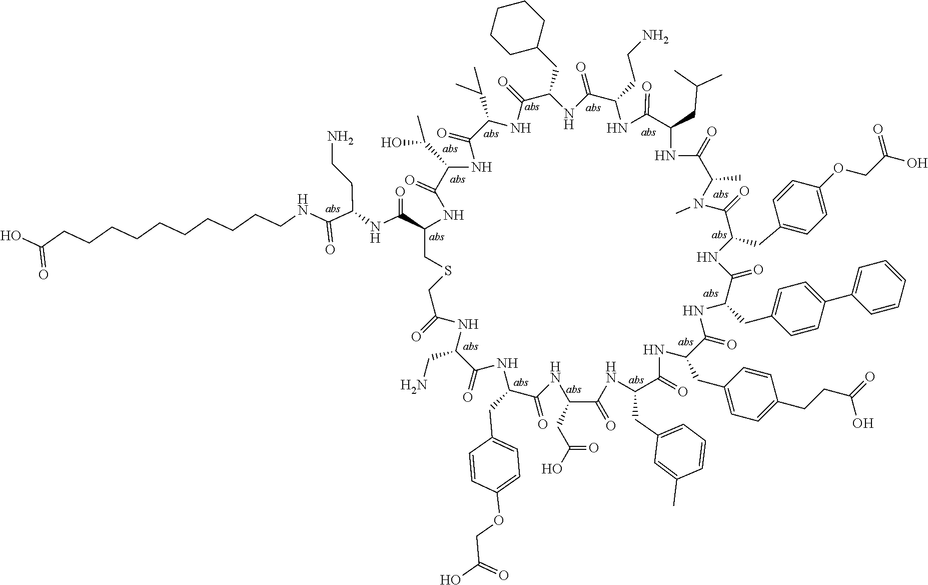 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(CCC(=O)O)cc3)NC2=O)c1]

Example 1262 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 42.4 mg, and its estimated purity by LCMS analysis was 94.8%. Analysis condition A: Retention time=1.53 min; ESI-MS(+) m/z [M+2H]²⁺: 1173.4.

### Preparation of Example 1263

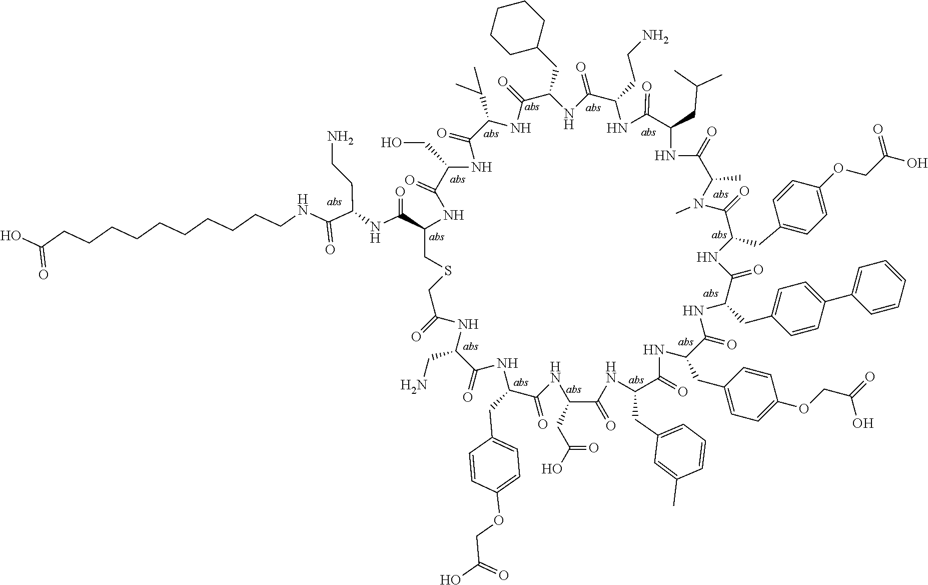 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1263 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 19% B, 19-59% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 100%. Analysis condition B: Retention time=1.71 min; ESI-MS(+) m/z [M+3H]³⁺: 778.2.

### Preparation of Example 1264

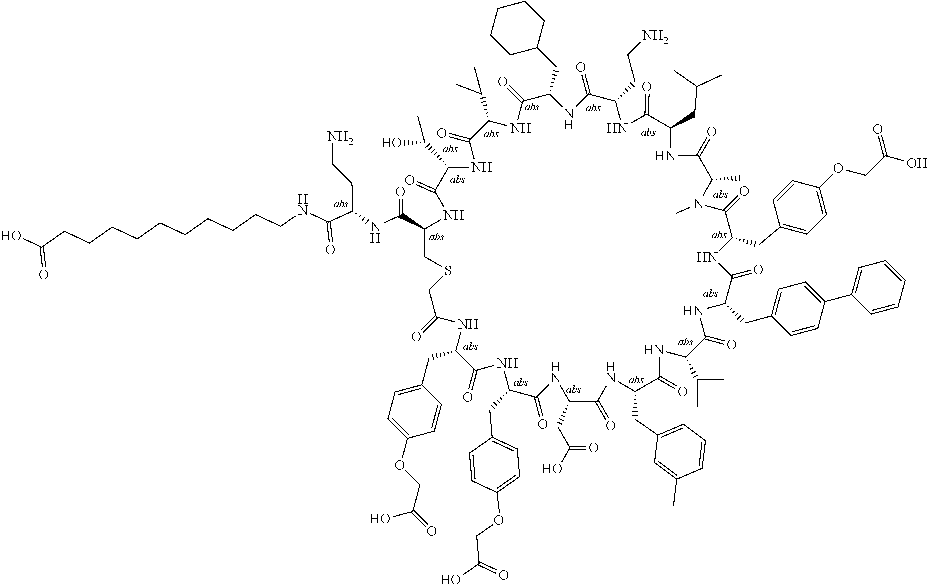 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 1264 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 22% B, 22-62% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 51.9 mg, and its estimated purity by LCMS analysis was 97.1%. Analysis condition A: Retention time=1.49 min; ESI-MS(+) m/z [M+2H]²⁺: 1180.1.

### Preparation of Example 1265

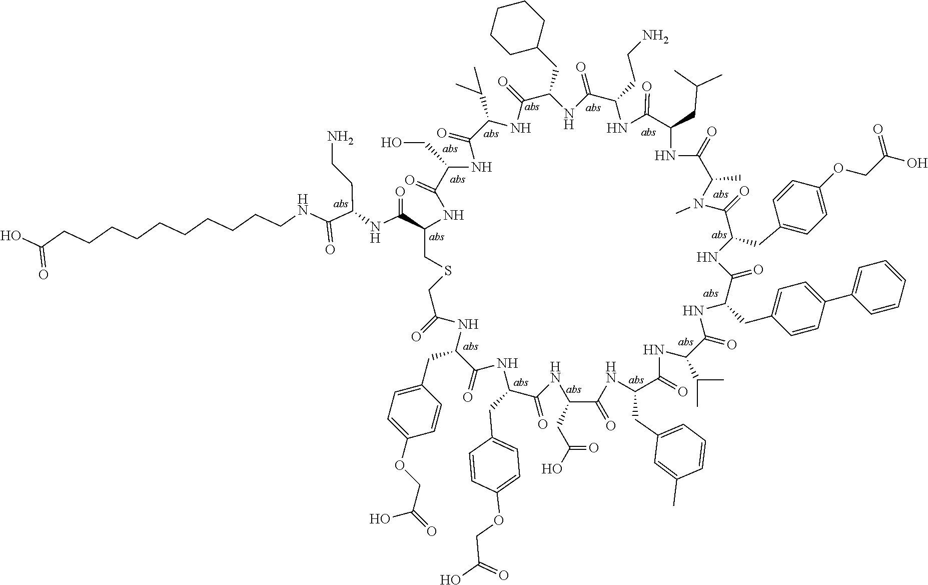 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 1265 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 24 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.3 mg, and its estimated purity by LCMS analysis was 95%. Analysis condition A: Retention time=1.17 min; ESI-MS(+) m/z [M+2H]²⁺: 1173.

### Preparation of Example 1266

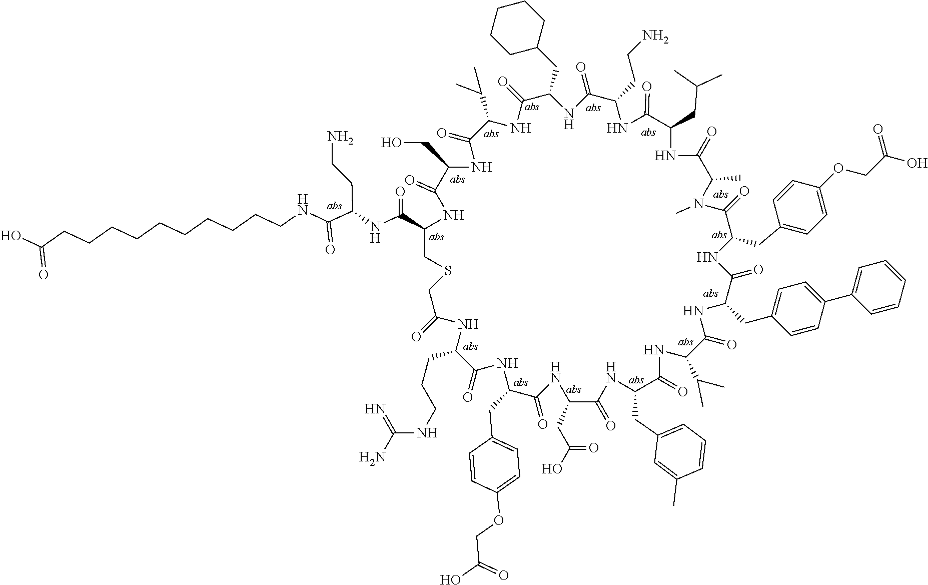 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 1266 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.8 mg, and its estimated purity by LCMS analysis was 98.9%. Analysis condition B: Retention time=1.66 min; ESI-MS(+) m/z [M+2H]²⁺: 1140.2.

### Preparation of Example 1267

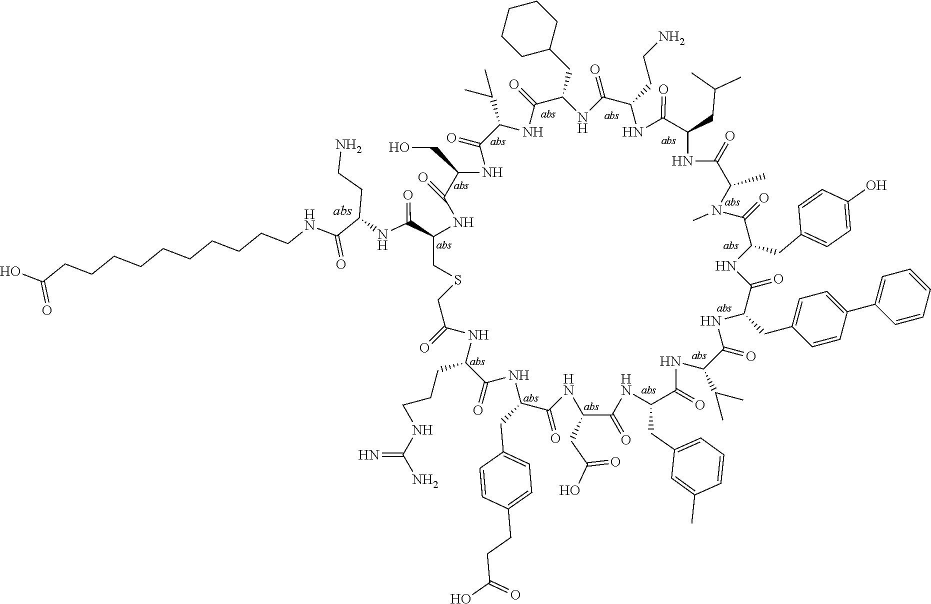 [SMILES: Cc1cccc(C[C@@H]2NC(=O)[C@H](CC(=O)O)NC(=O)[C@H](Cc3ccc(CCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1267 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.9 mg, and its estimated purity by LCMS analysis was 94.8%. Analysis condition B: Retention time=1.65 min; ESI-MS(+) m/z [M+2H]²⁺: 1111.1.

### Preparation of Example 1268

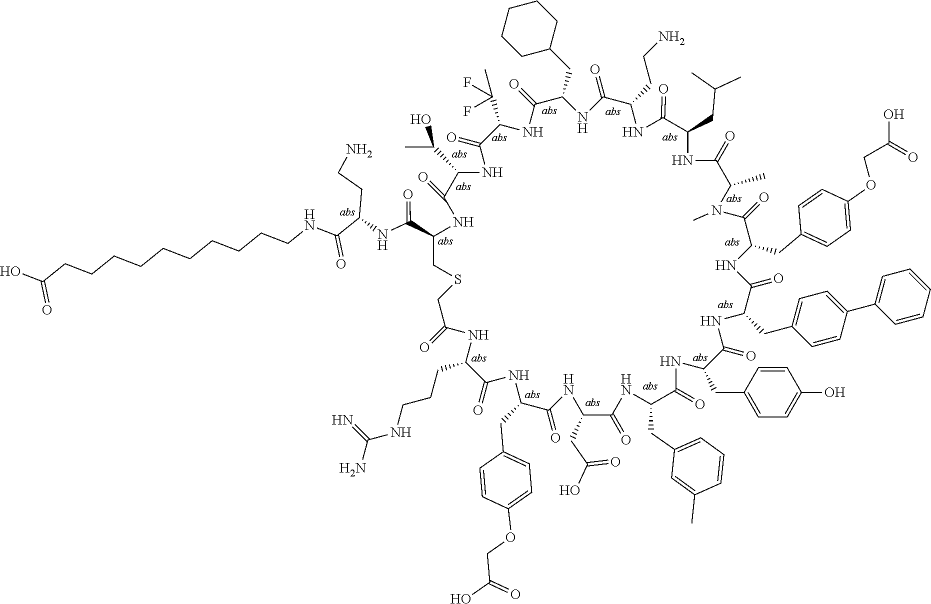 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)(F)F)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Example 1268 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with (R)-2-((((9H-fluoren-9-yl)methoxy)carbonyl)amino)-3-fluoro-3-methylbutanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 62.1 mg, and its estimated purity by LCMS analysis was 92.5%. Analysis condition A: Retention time=1.7 min; ESI-MS(+) m/z [M+3H]³⁺: 793.

### Preparation of Example 1269

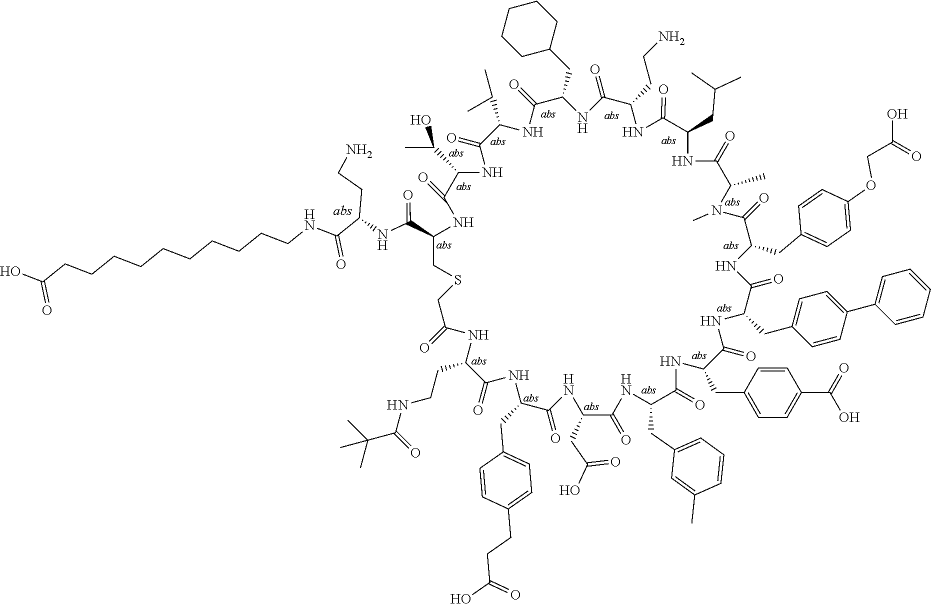 [SMILES: Cc1cccc(C[C@@H]2NC(=O)[C@H](CC(=O)O)NC(=O)[C@H](Cc3ccc(CCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1269 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-Dab(CotBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.2 mg, and its estimated purity by LCMS analysis was 95.9%. Analysis condition B: Retention time=1.84 min; ESI-MS(+) m/z [M+3H]³⁺: 805.4.

### Preparation of Example 1270

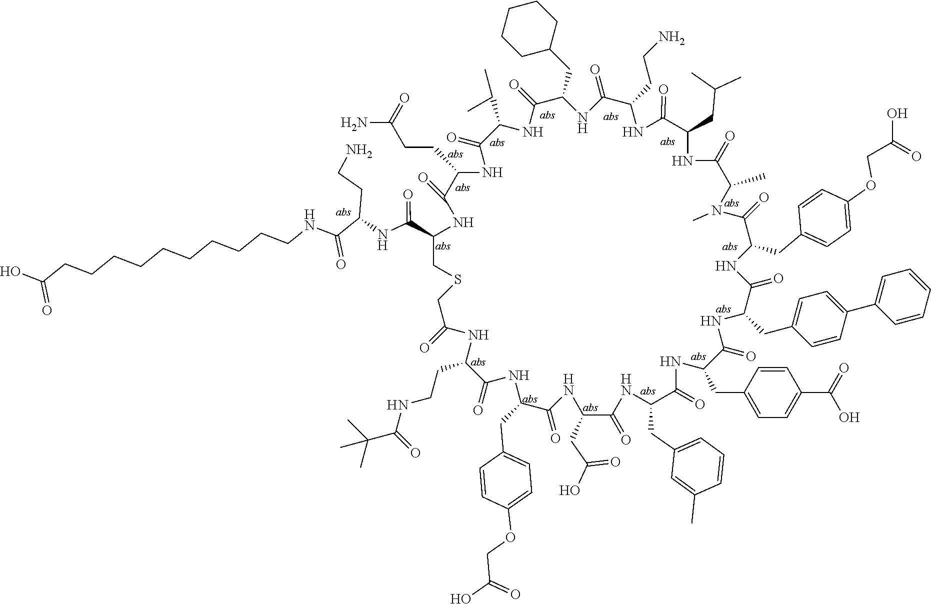 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1270 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23 mg, and its estimated purity by LCMS analysis was 95.3%. Analysis condition: A Retention time=1.58 min; ESI-MS(+) m/z [M+2H]²⁺: 1220.9.

### Preparation of Example 1271

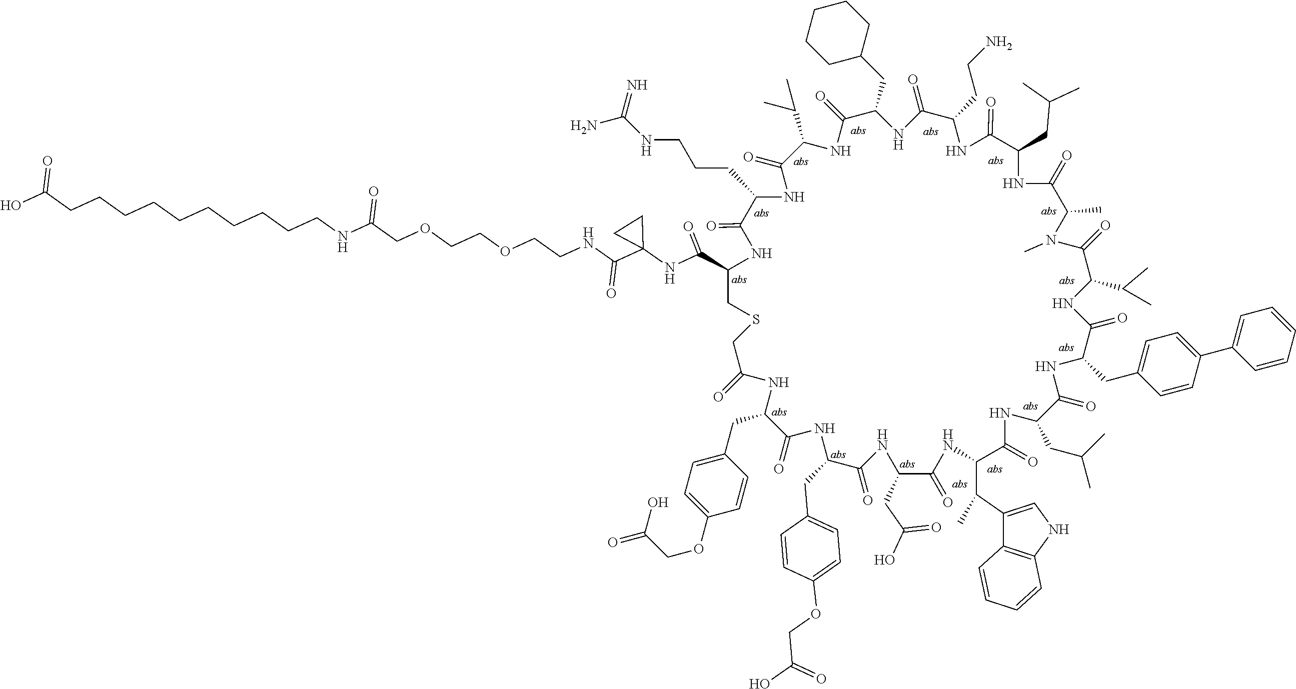 [SMILES: CC(C)C[C@@H]1NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NC2(C(=O)NCCOCCOCC(=O)NCCCCCCCCCCC(=O)O)CC2)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1271 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 28% B, 28-68% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 27.6 mg, and its estimated purity by LCMS analysis was 98.3%. Analysis condition A: Retention time=1.74 min; ESI-MS(+) m/z [M+2H]²⁺: 1237.2.

### Preparation of Example 1272

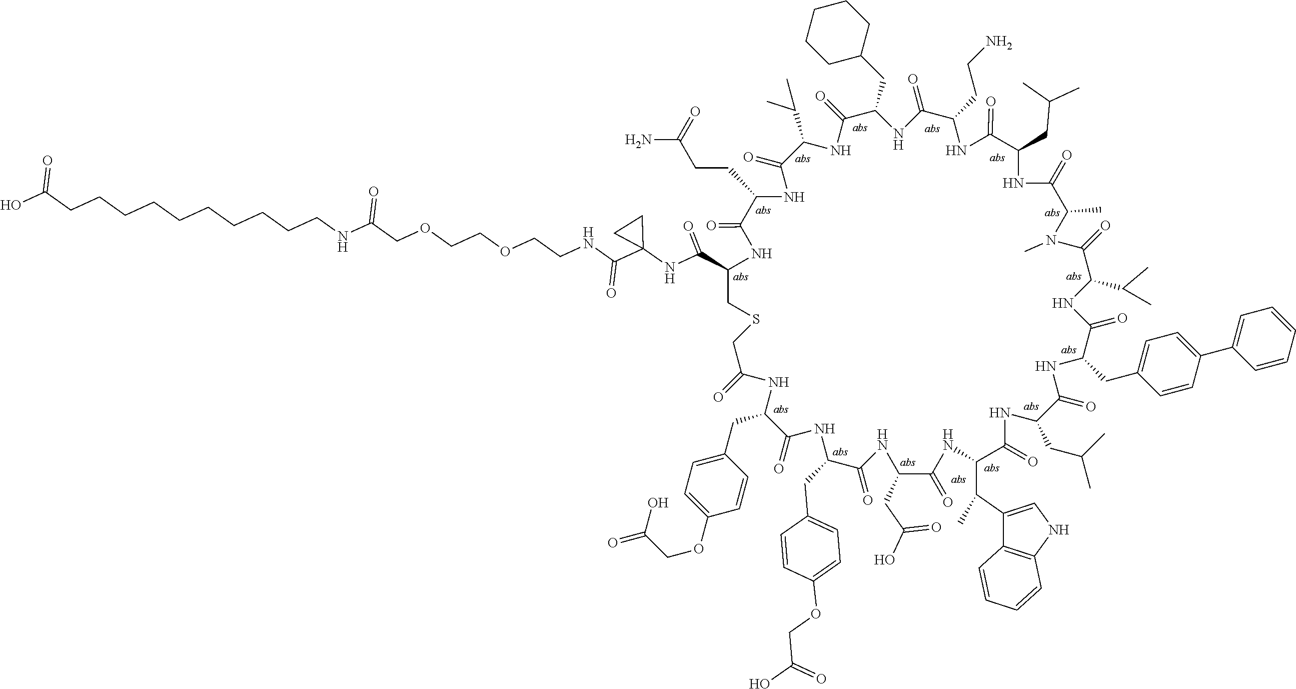 [SMILES: CC(C)C[C@@H]1NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NC2(C(=O)NCCOCCOCC(=O)NCCCCCCCCCCC(=O)O)CC2)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1272 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with 1-((((9H-fluoren-9-yl)methoxy)carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 51.4 mg, and its estimated purity by LCMS analysis was 99.5%. Analysis condition B: Retention time=2.04 min; ESI-MS(+) m/z [M+2H]²⁺: 1223.2.

### Preparation of Example 1273

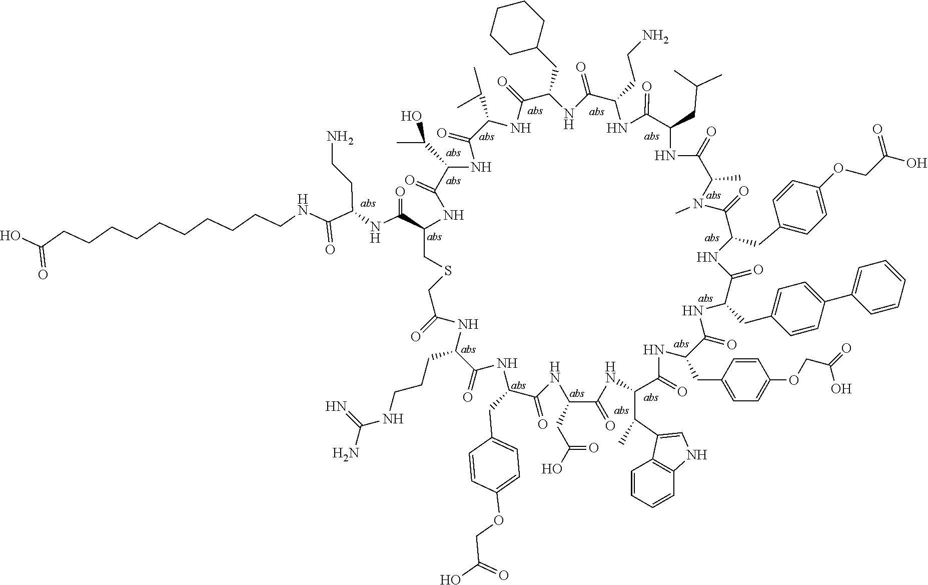 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1273 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 68.2 mg, and its estimated purity by LCMS analysis was 90.3%. Analysis condition A: Retention time=1.55 min; ESI-MS(+) m/z [M+2H]²⁺: 1228.

### Preparation of Example 1274

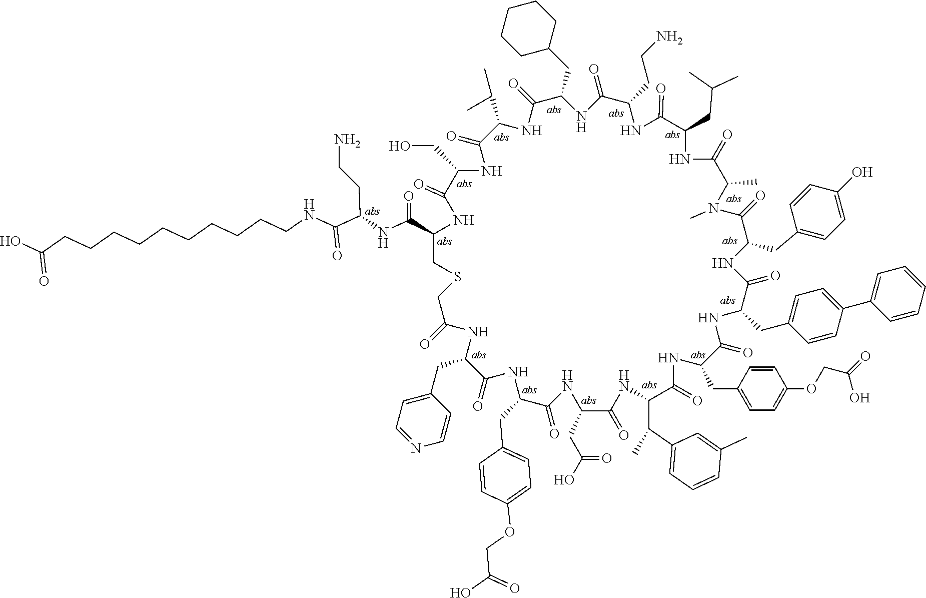 [SMILES: Cc1cccc([C@H](C)[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1274 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 15% B, 15-59% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 93.9%. Analysis condition B: Retention time=1.77 min; ESI-MS(+) m/z [M+3H]³⁺: 779.

### Preparation of Example 1275

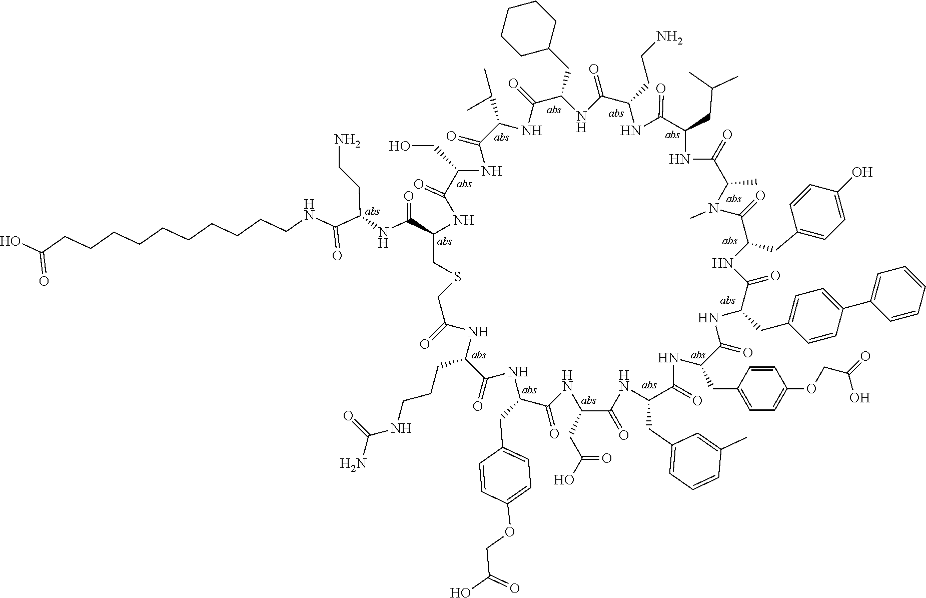 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(N)=O)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1275 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.8 mg, and its estimated purity by LCMS analysis was 93.9%. Analysis condition B: Retention time=2.02 min; ESI-MS(+) m/z [M+2H]²⁺: 1173.1.

### Preparation of Example 1276

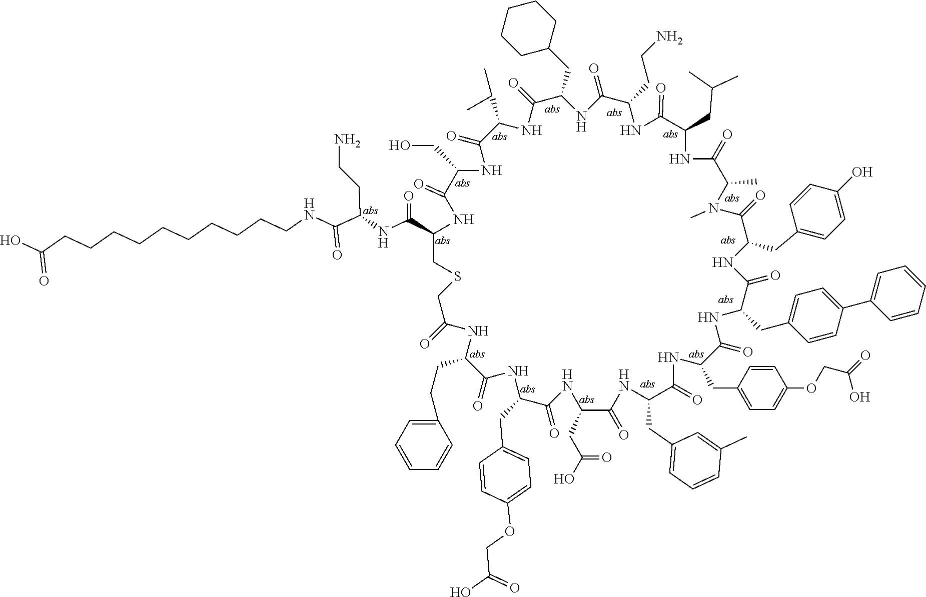 [SMILES: Cc1cccc(C[C@@H]2NC(=O)[C@H](CC(=O)O)NC(=O)[C@H](Cc3ccc(OCC(=O)O)cc3)NC(=O)[C@H](CCc3ccccc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1276 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-homo-Phe-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 34% B, 34-64% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 32.5 mg, and its estimated purity by LCMS analysis was 93.6%. Analysis condition B: Retention time=1.94 min; ESI-MS(+) m/z [M+2H]²⁺: 1175.

### Preparation of Example 1277

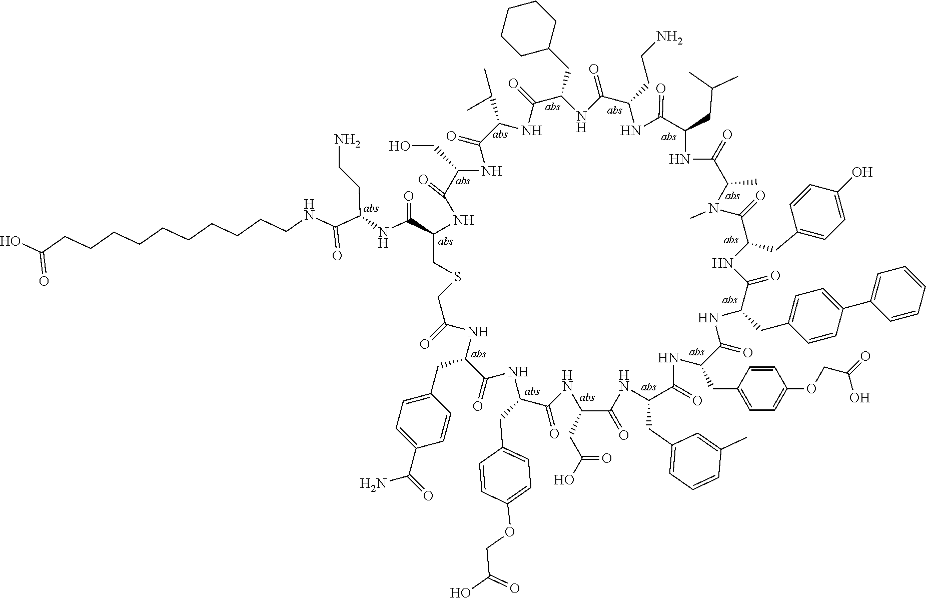 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1277 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-Phe(4-CONH₂); “Symphony X Chloroacetic Anhydride coupling procedure”, “Symphony X Final rinse and dry procedure”,
“Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 13% B, 13-58% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 28% B, 28-68% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-pm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.6 mg, and its estimated purity by LCMS analysis was 98.4%. Analysis condition A: Retention time=1.52 min; ESI-MS(+) m/z [M+2H]²⁺: 1189.3.

### Preparation of Example 1278

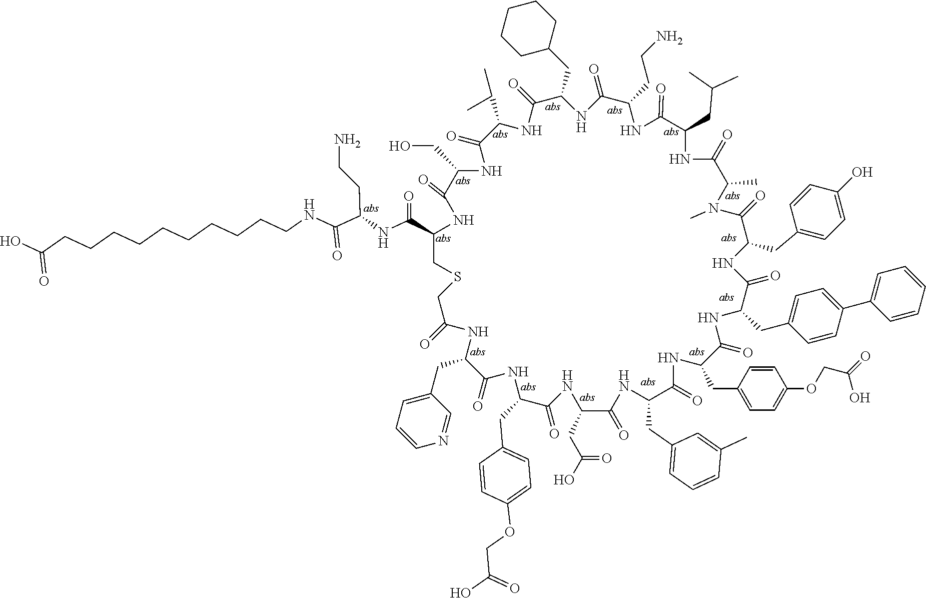 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1278 was prepared, using 2-Chlorotrityl resin pre-loaded with 11-((((9H-fluoren-9-yl)methoxy)carbonyl)amino)undecanoic acid on a 25 □mol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-Ala(3-pyridyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 15% B, 15-59% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 90.6%. Analysis condition B: Retention time=1.78 min; ESI-MS(+) m/z [M+3H]³⁺: 779.

### Preparation of Example 1279

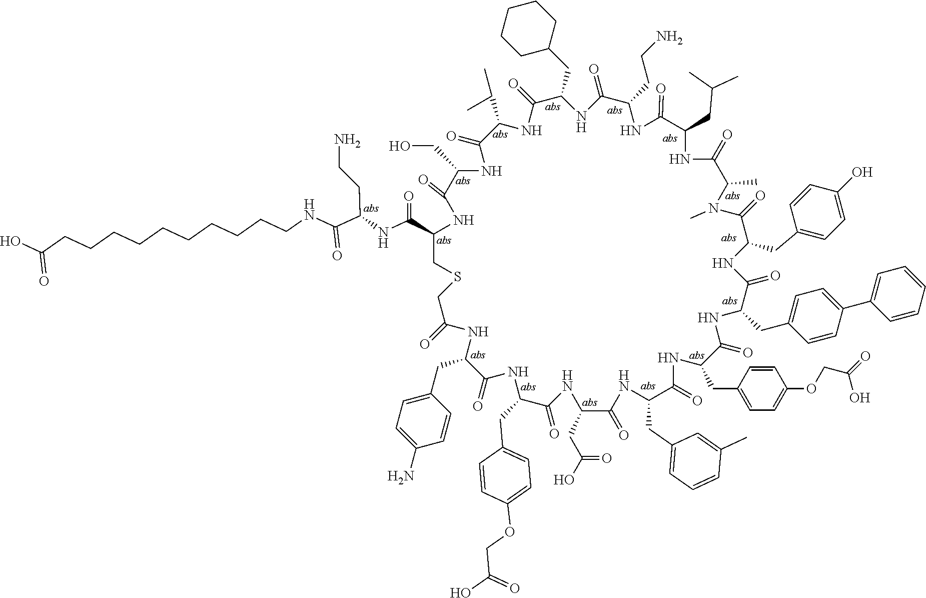 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(N)cc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1279 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Fmoc-Phe-(4-NHBoc); “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 21% B, 21-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.9 mg, and its estimated purity by LCMS analysis was 91.5%. Analysis condition B: Retention time=1.77 min; ESI-MS(+) m/z [M+2H]²⁺: 1175.1.

### Preparation of Example 1280

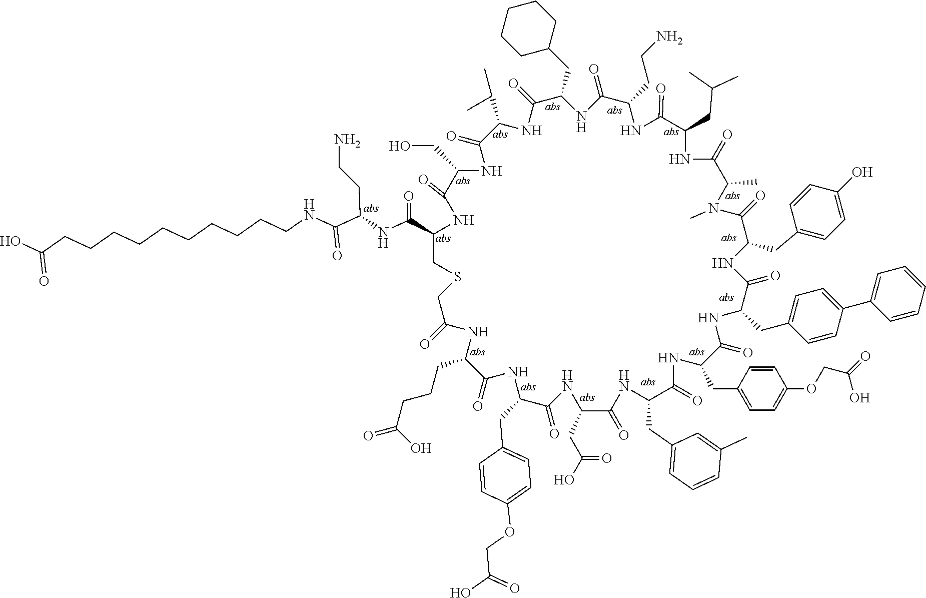 [SMILES: Cc1cccc(C[C@@H]2NC(=O)[C@H](CC(=O)O)NC(=O)[C@H](Cc3ccc(OCC(=O)O)cc3)NC(=O)[C@H](CCCC(=O)O)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1280 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with (S)-2-((((9H-fluoren-9-yl)methoxy)carbonyl)amino)-6-(tert-butoxy)-6-oxohexanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 13% B, 13-57% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.9 mg, and its estimated purity by LCMS analysis was 88.8%. Analysis condition B: Retention time=1.89 min; ESI-MS(+) m/z [M+2H]²⁺: 1166.

### Preparation of Example 1281

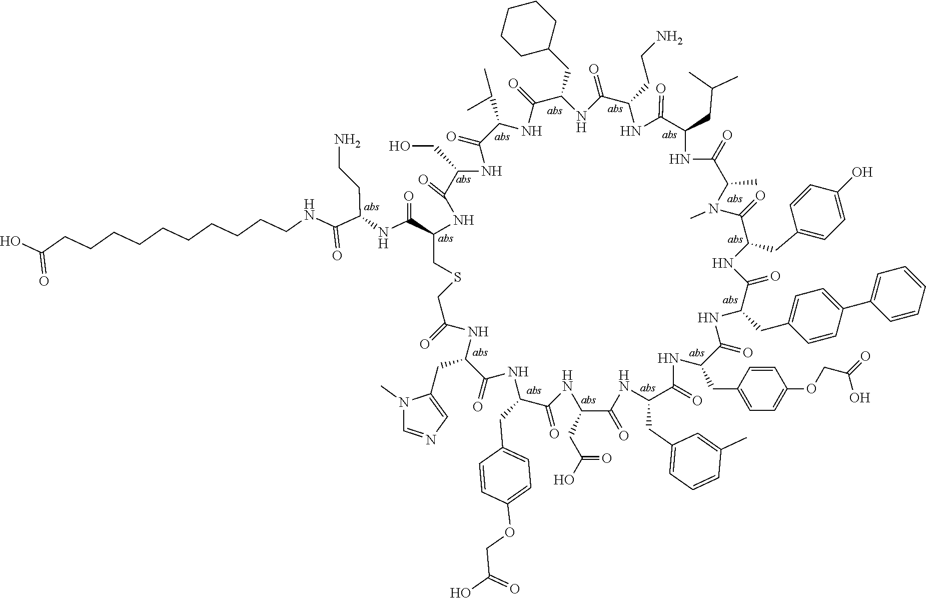 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 1281 was prepared, using 2-Chlorotrityl resin pre-loaded with 11-((((9H-fluoren-9-yl)methoxy)carbonyl)amino)undecanoic acid on a 25 μmol scale, following the general synthetic sequence described for the preparation of Example 1000 composed of the following general procedures: “Symphony X Resin-swelling procedure”, “Symphony X Single-coupling procedure”, or ““Symphony X Double-coupling procedure”, “Symphony X Single-Coupling Manual Addition Procedure A” was followed with Na-(((9H-fluoren-9-yl)methoxy)carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 15% B, 15-59% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 90.7%. Analysis condition B: Retention time=1.78 min; ESI-MS(+) m/z [M+2H]²⁺: 1170.2.

### Preparation of Example 1282

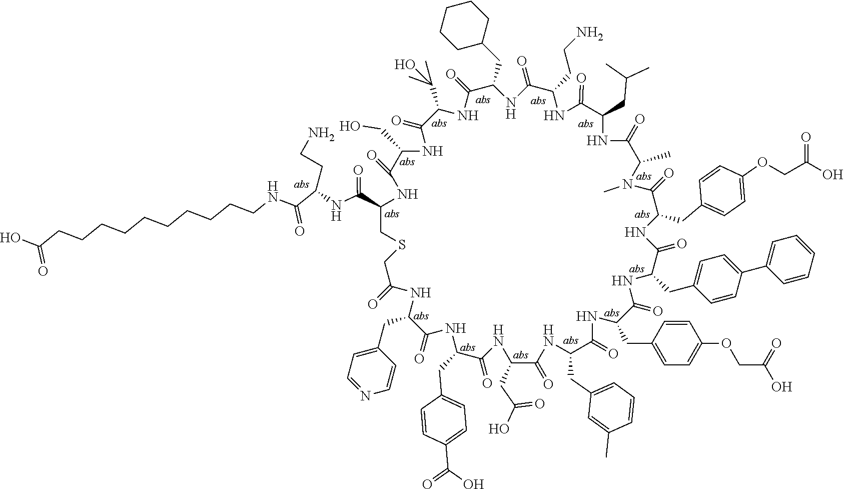 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1282 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 24.6 mg, and its estimated purity by LCMS analysis was 97.2%. Analysis condition A: Retention time=1.52 min; ESI-MS(+) m/z [M+2H]²⁺: 1190.1.

### Preparation of Example 1283

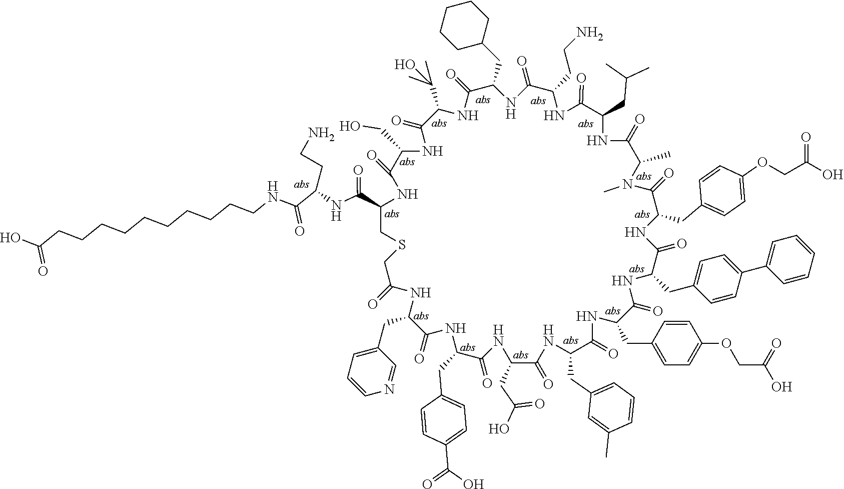 [SMILES: Cc1cccc(C[C@@H]2NC(=O)[C@H](CC(=O)O)NC(=O)[C@H](Cc3ccc(C(=O)O)cc3)NC(=O)[C@H](Cc3cccnc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1283 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Ala(2-pyridyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 31.8 mg, and its estimated purity by LCMS analysis was 91.9%. Analysis condition A: Retention time=1.55 min; ESI-MS(+) m/z [M+2H]²⁺: 1191.

### Preparation of Example 1284

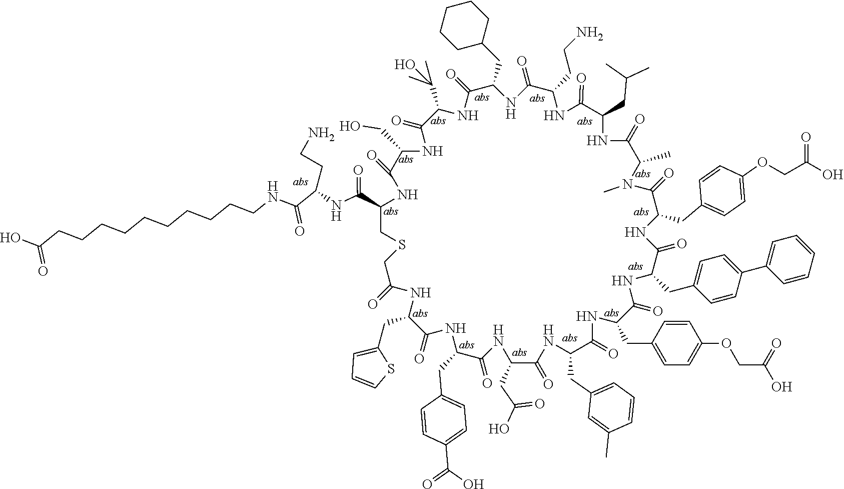 [SMILES: Cc1cccc(C[C@@H]2NC(=O)[C@H](CC(=O)O)NC(=O)[C@H](Cc3ccc(C(=O)O)cc3)NC(=O)[C@H](Cc3cccs3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1284 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Ala(2-thiophene)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 26% B, 26-66% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.3 mg, and its estimated purity by LCMS analysis was 95.1%. Analysis condition A: Retention time=1.56 min; ESI-MS(+) m/z [M+2H]²⁺: 1193.1.

### Preparation of Example 1285

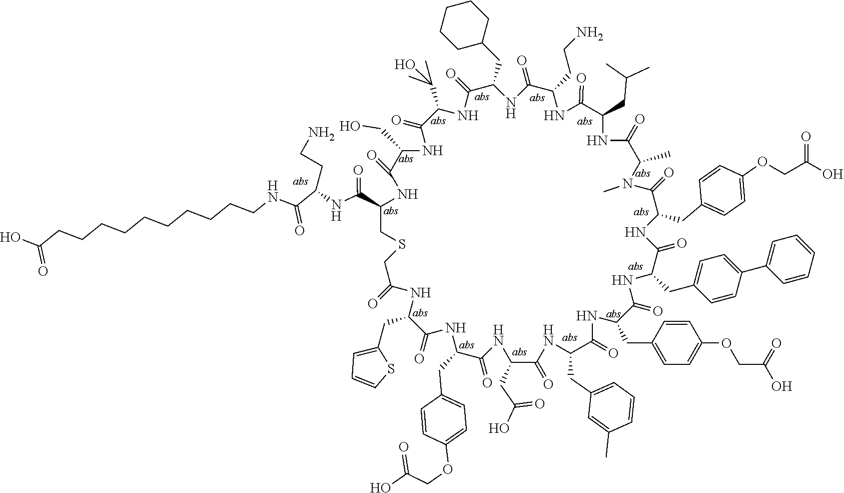 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccs3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1285 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Ala(2-thiophene)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.100 trifluoroacetic acid; Mobile Phase B: 95:5 acetonitrile:water with 0.1%0 trifluoroacetic acid; Gradient: a 0-minute hold at 29% B, 29-69% B over 20 minutes, then a 10-minute hold at 100% B; Flow Rate: 20 mL/min; Column Temperature: 25 C. Fraction collection was triggered by MS signals. Fractions containing the desired product were combined and dried via centrifugal evaporation. The yield of the product was 69.1 mg, and its estimated purity by LCMS analysis was 90.2%. Analysis condition A: Retention time=1.63 min; ESI-MS(+) m/z [M+2H]²⁺: 1207.9.

### Preparation of Example 1286

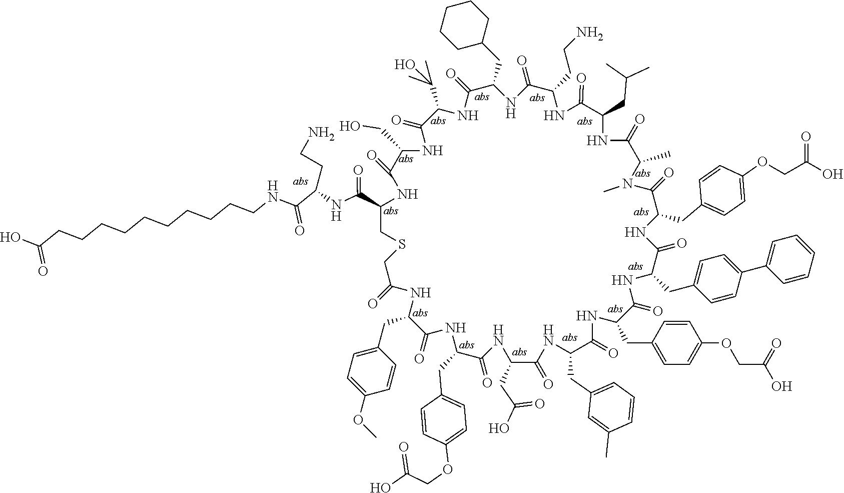 [SMILES: COc1ccc(C[C@@H]2NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC2=O)cc1]

Example 1286 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 26% B, 26-66% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.6 mg, and its estimated purity by LCMS analysis was 95.6%. Analysis condition A: Retention time=1.62 min; ESI-MS(+) m/z [M+2H]²⁺: 1219.9.

### Preparation of Example 1287

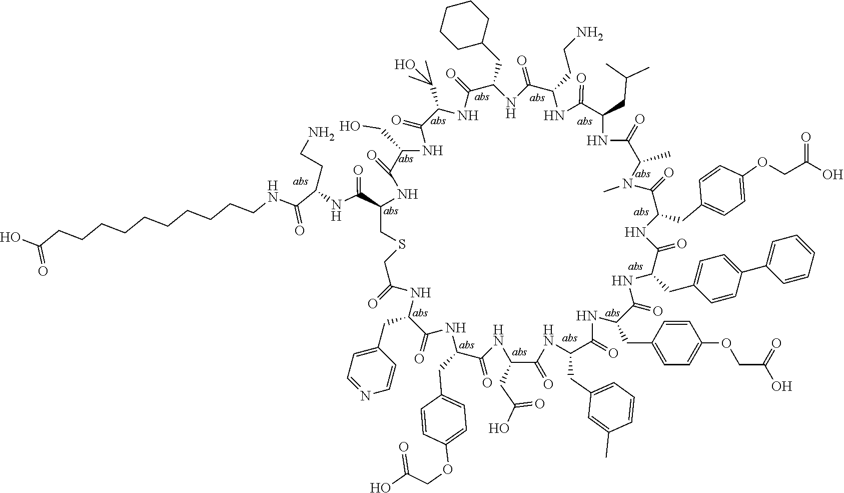 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1287 was prepared, using 2-Chorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.100 trifluoroacetic acid; Gradient: a 0-minute hold at 21% B, 21-61% B over 20 minutes, then a 0-minute hold at 100% a B; Flow Rate: 20 mL/min; Column Temperature: 25 C. Fraction collection was triggered by MS and UV signals. Fractions containing the desired product were combined and dried via centrifugal evaporation. The yield of the product was 22.2 mg, and its estimated purity by LCMS analysis was 96.1%. Analysis condition A: Retention time=1.44 min; ESI-MS(+v) m/z [M+2H]²⁺: 1206.

### Preparation of Example 1288

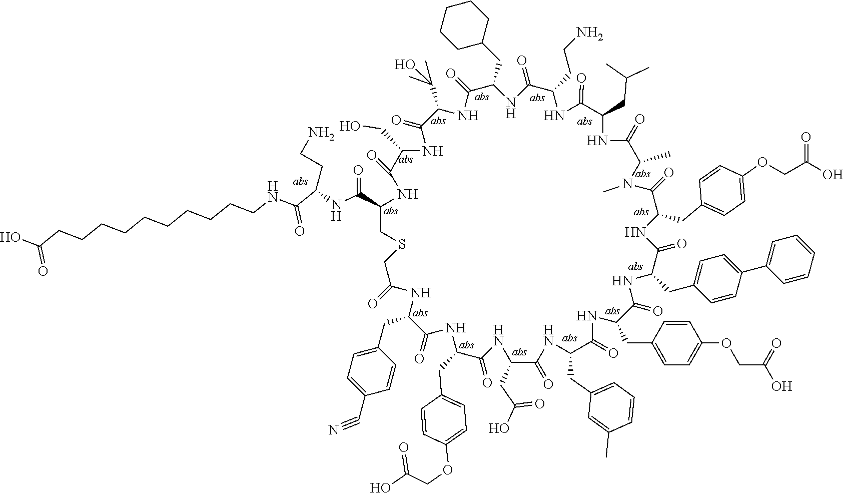 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C#N)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1288 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(3-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 26% B, 26-66% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14.8 mg, and its estimated purity by LCMS analysis was 89.9%. Analysis condition A: Retention time=1.6 min; ESI-MS(+) m/z [M+2H]²⁺: 1217.2.

### Preparation of Example 1289

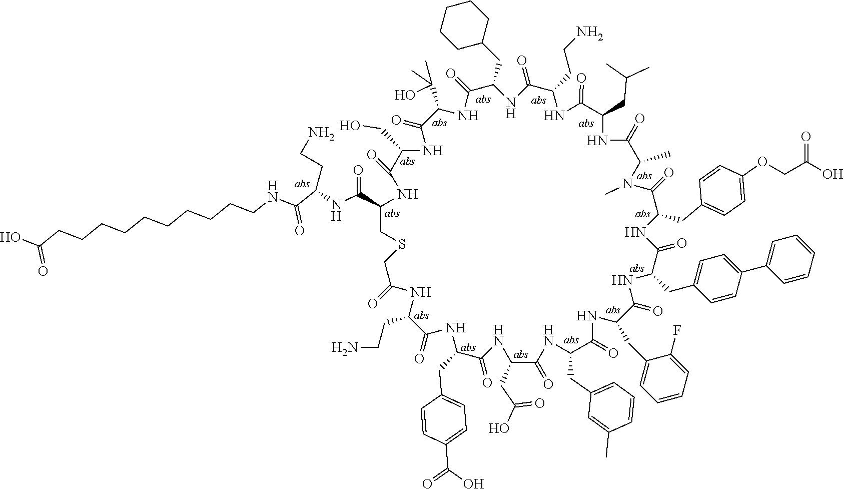 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccccc3F)NC2=O)c1]

Example 1289 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(2-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 24% B, 24-69% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12 mg, and its estimated purity by LCMS analysis was 92.5%. Analysis condition A: Retention time=1.76 min; ESI-MS(+) m/z [M+3H]²⁺: 759.1.

### Preparation of Example 1290

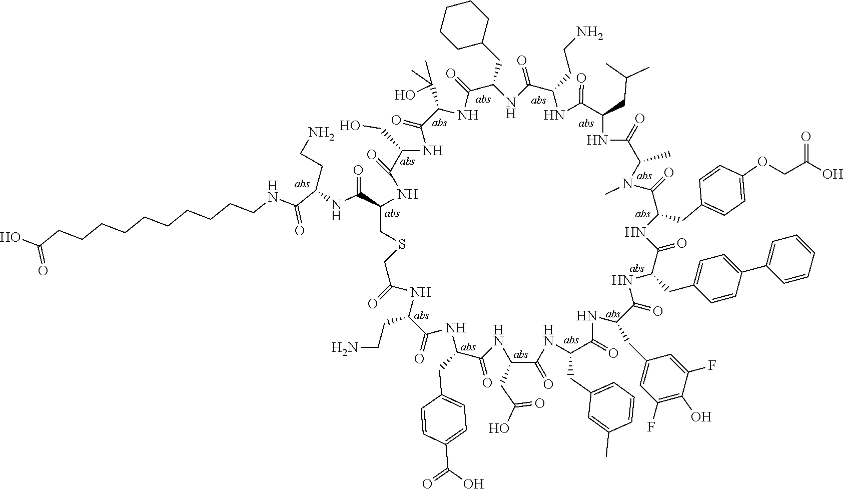 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cc(F)c(O)c(F)c3)NC2=O)c1]

Example 1290 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(2,6-diF)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 22% B, 22-62% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.9 mg, and its estimated purity by LCMS analysis was 97.2%. Analysis condition A: Retention time=1.61 min; ESI-MS(+) m/z [M+3H]³⁺: 770.5.

### Preparation of Example 1291

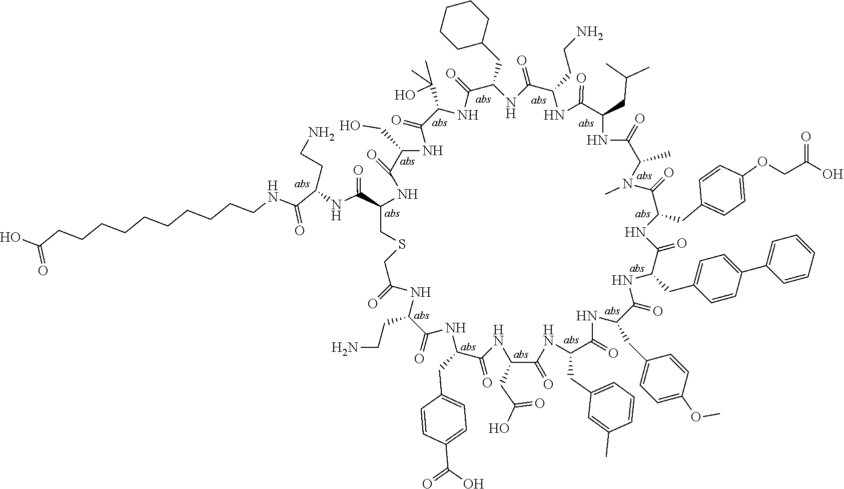 [SMILES: COc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC2=O)cc1]

Example 1291 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 24% B, 24-64% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23.5 mg, and its estimated purity by LCMS analysis was 94.9%. Analysis condition A: Retention time=1.74 min; ESI-MS(+) m/z [M+2H]²⁺: 1144.1.

### Preparation of Example 1292

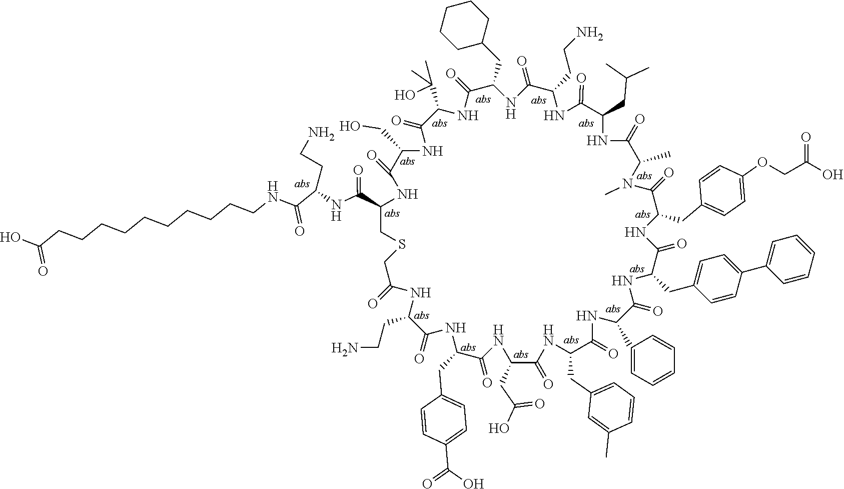 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](c3ccccc3)NC2=O)c1]

Example 1292 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-pheylglycine-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 24% B, 24-64% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 18% B, 18-58% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 95.6%. Analysis condition A: Retention time=1.64 min; ESI-MS(+) m/z [M+2H]²⁺: 1122.5.

### Preparation of Example 1293

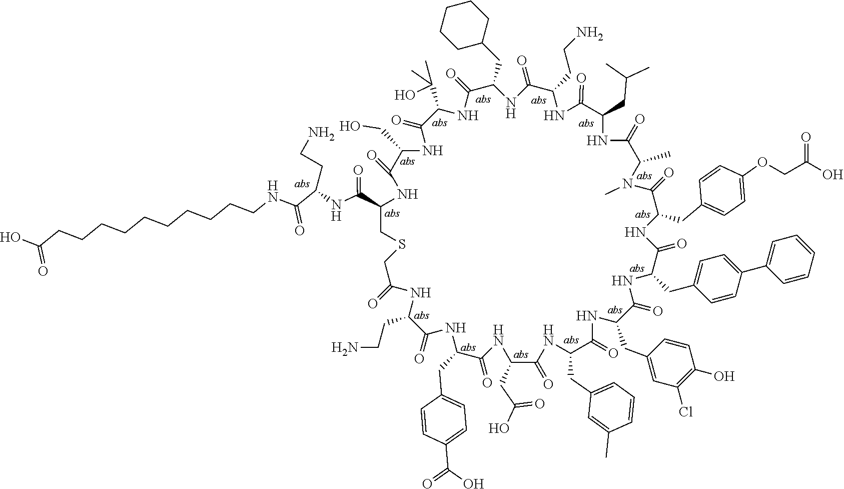 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(O)c(Cl)c3)NC2=O)c1]

Example 1293 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(3-Cl)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 2.7 mg, and its estimated purity by LCMS analysis was 90.9%. Analysis condition B: Retention time=1.9 min; ESI-MS(+) m/z [M+3H]³⁺: 770.2.

### Preparation of Example 1294

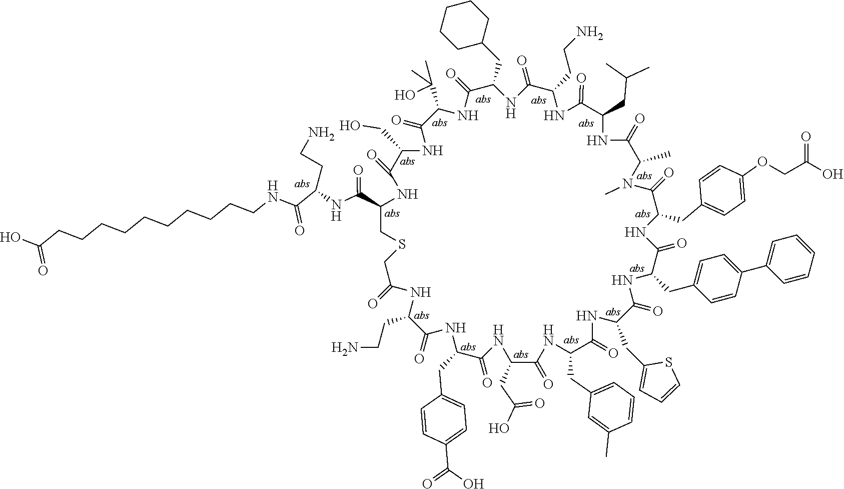 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccs3)NC2=O)c1]

Example 1294 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Ala(2-thiophene)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 20% B, 20-60% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 10 mg, and its estimated purity by LCMS analysis was 93.3%. Analysis condition A: Retention time=1.61 min; ESI-MS(+) m/z [M+2H]²⁺: 1132.1.

### Preparation of Example 1295

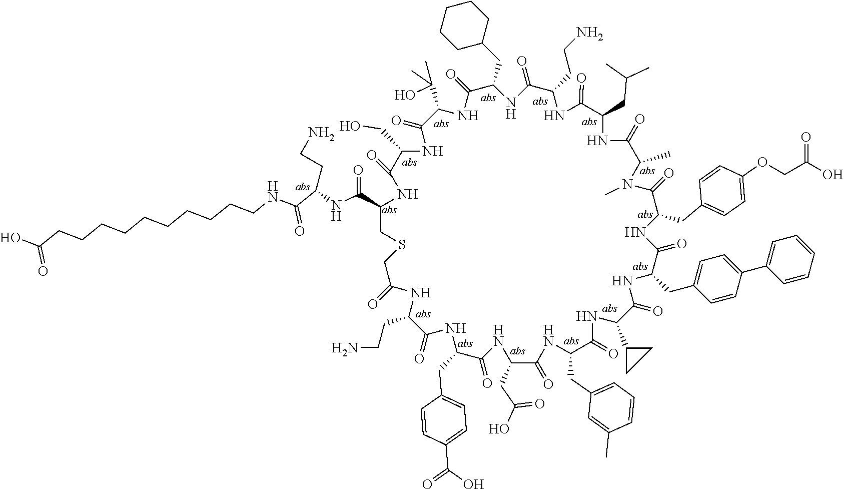 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C3CC3)NC2=O)c1]

Example 1295 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-cyclopropylglycine-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 23% B, 23-63% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 100%. Analysis condition A: Retention time=1.66 min; ESI-MS(+) m/z [M+2H]²⁺: 1104.4.

### Preparation of Example 1296

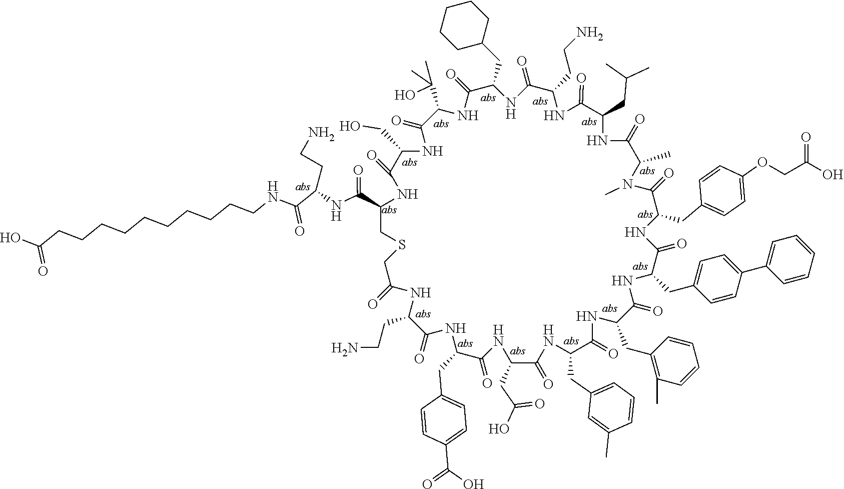 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccccc3C)NC2=O)c1]

Example 1296 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(2-Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 26% B, 26-66% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.2 mg, and its estimated purity by LCMS analysis was 95.7%. Analysis condition A: Retention time=1.82 min; ESI-MS(+) m/z [M+3H]³⁺: 758.3.

### Preparation of Example 1297

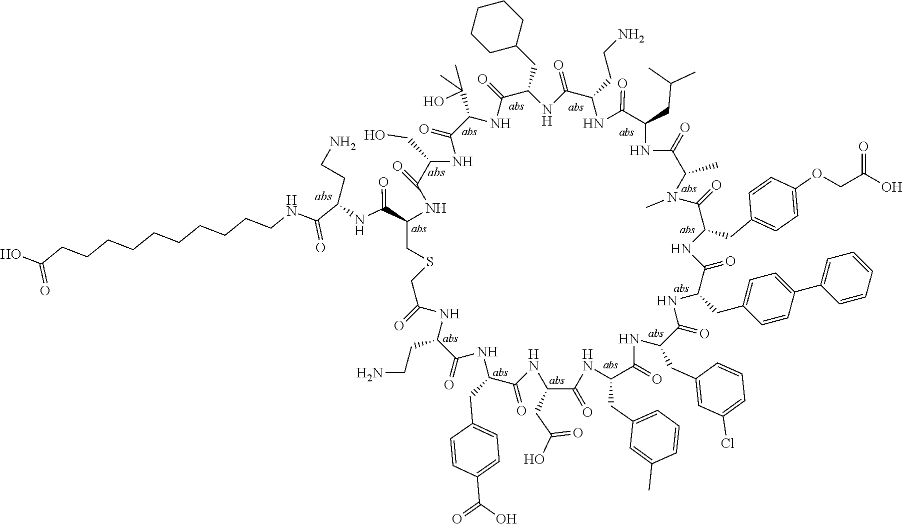 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(Cl)c3)NC2=O)c1]

Example 1297 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(3-Cl)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 92.1%. Analysis condition A: Retention time=1.72 min; ESI-MS(+) m/z [M+2H]²⁺: 1146.2.

### Preparation of Example 1298

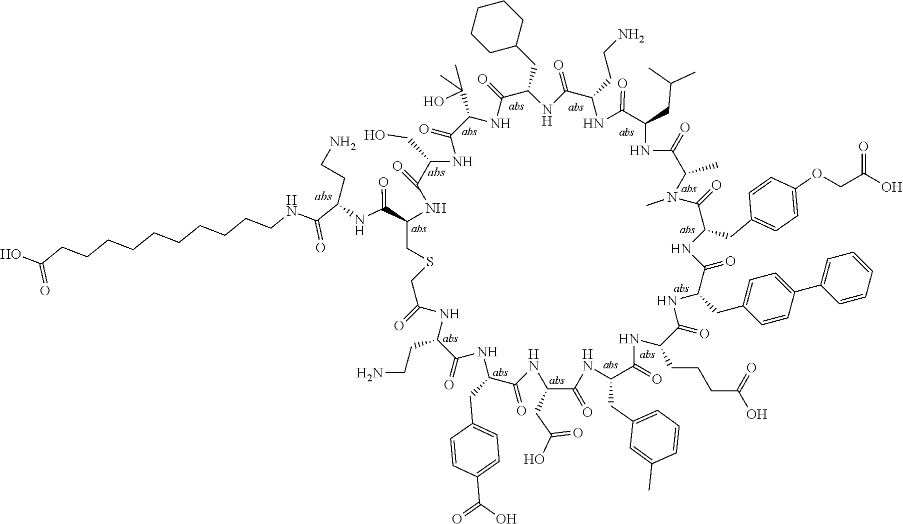 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCC(=O)O)NC2=O)c1]

Example 1298 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with (S)-2-((((9H-fluoren-9-yl)methoxy)carbonyl)amino)-6-(tert-butoxy)-6-oxohexanoic acid, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 14% B, 14-54% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.5 mg, and its estimated purity by LCMS analysis was 92.1%. Analysis condition B: Retention time=1.82 min; ESI-MS(+) m/z [M+3H]²⁺: 752.2.

### Preparation of Example 1299

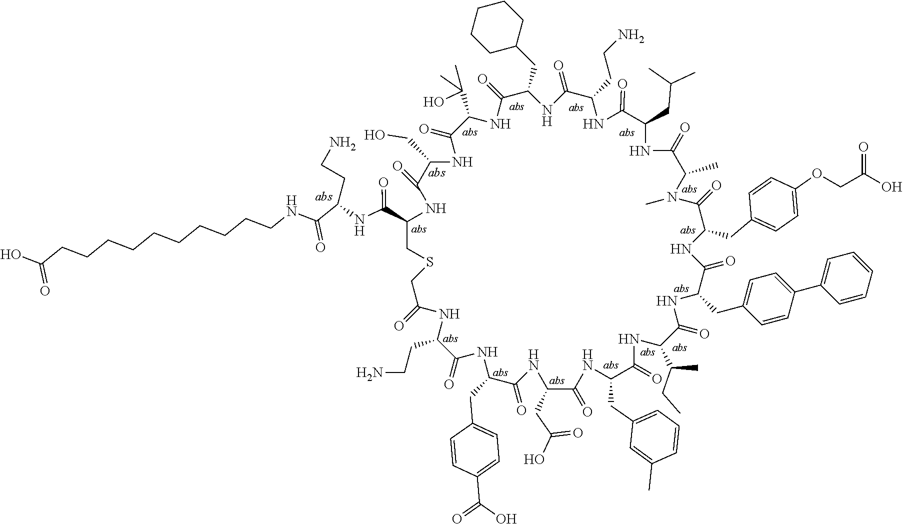 [SMILES: CC[C@H](C)[C@@H]1NC(=O)[C@H](Cc2cccc(C)c2)NC(=O)[C@H](CC(=O)O)NC(=O)[C@H](Cc2ccc(C(=O)O)cc2)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC1=O]

Example 1299 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 20% B, 20-60% B over 25 minutes, then a 0-minute hold at 100% a B; Flow Rate: 20 mL/min; Column Temperature: 25 C. Fraction collection was triggered by MS signals. Fractions containing the desired product were combined and dried via centrifugal evaporation. The yield of the product was 5.2 mg, and its estimated purity by LCMS analysis was 100%. Analysis condition A: Retention time=1.6 min; ESI-MS(+) m/z [M+2H]²⁺: 1112.2.

### Preparation of Example 1300

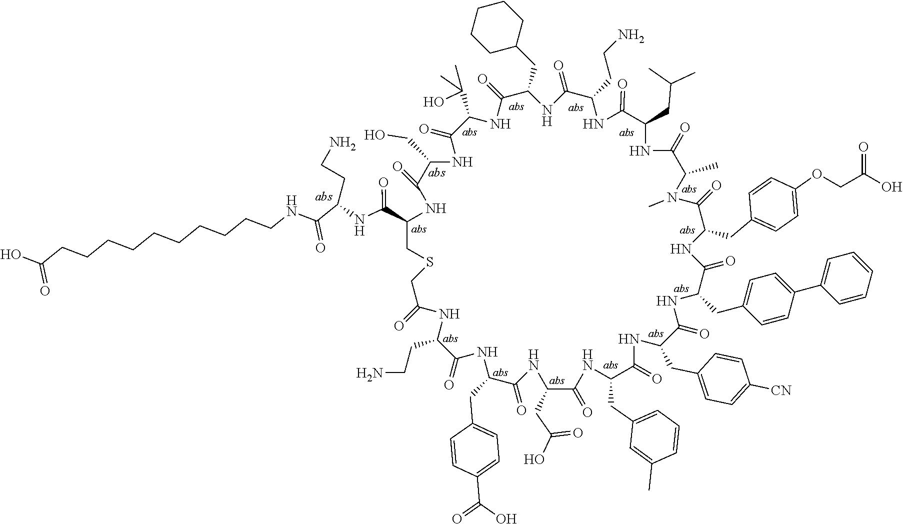 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C#N)cc3)NC2=O)c1]

Example 1300 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 19% B, 19-59% B over 27 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.2 mg, and its estimated purity by LCMS analysis was 93.9%. Analysis condition B: Retention time=1.79 min; ESI-MS(+) m/z [M+2H]²⁺: 1142.2.

### Preparation of Example 1301

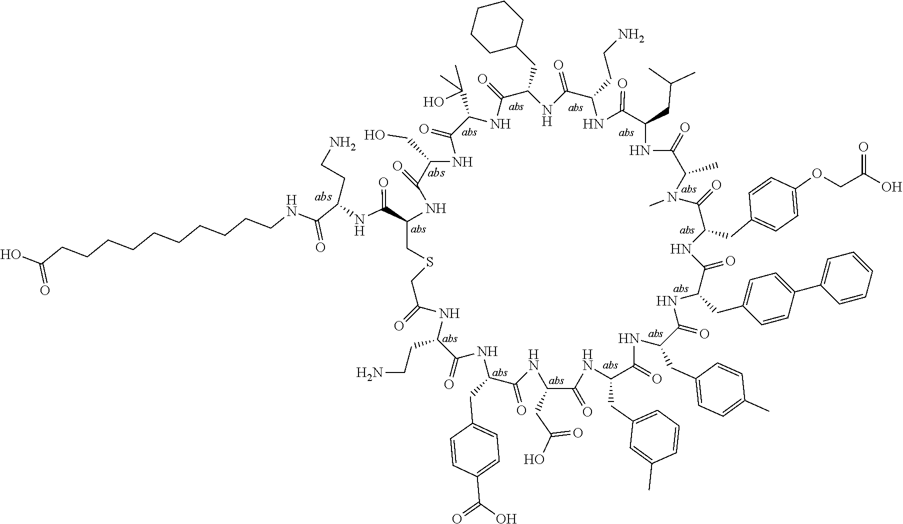 [SMILES: Cc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC2=O)cc1]

Example 1301 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed Fmoc-Phe(4-Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 20% B, 20-60% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.3 mg, and its estimated purity by LCMS analysis was 97.3%. Analysis condition B: Retention time=1.85 min; ESI-MS(+) m/z [M+3H]³⁺: 758.1.

### Preparation of Example 1302

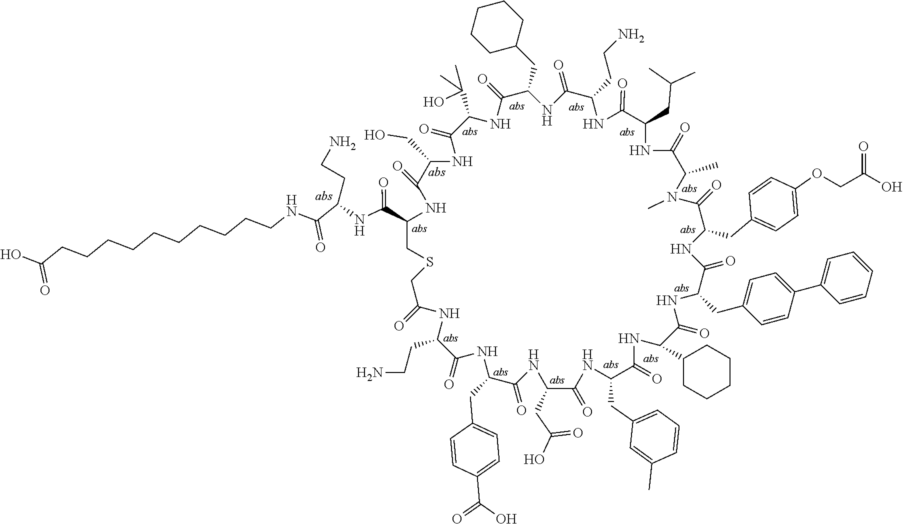 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C3CCCCC3)NC2=O)c1]

Example 1302 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-cyclohexylglycine-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 25% B, 25-65% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.2 mg, and its estimated purity by LCMS analysis was 98.7%. Analysis condition A: Retention time=1.71 min; ESI-MS(+) m/z [M+2H]²⁺: 1125.3.

### Preparation of Example 1303

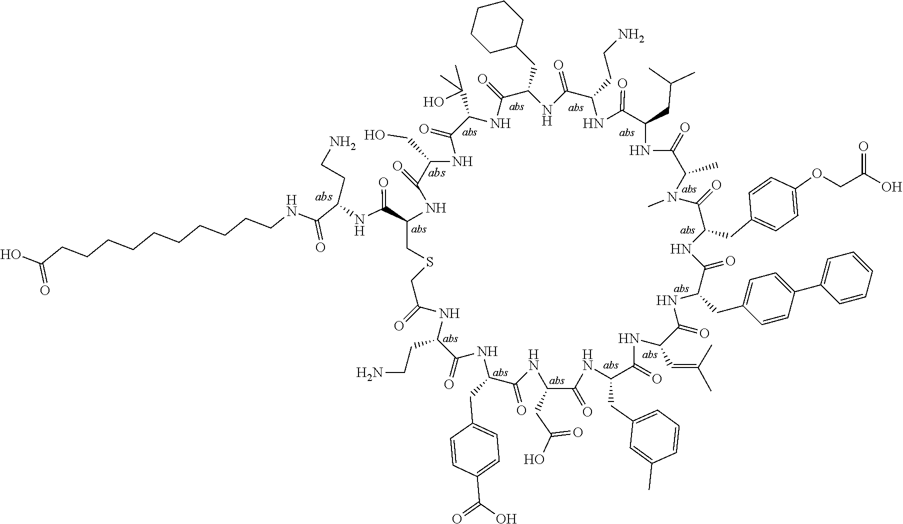 [SMILES: CC(C)=C[C@@H]1NC(=O)[C@H](Cc2cccc(C)c2)NC(=O)[C@H](CC(=O)O)NC(=O)[C@H](Cc2ccc(C(=O)O)cc2)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC1=O]

Example 1303 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-dehydro-Leu-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.100 trifluoroacetic acid; Mobile Phase B: 95:5 acetonitrile:water with 0.1% trifluoroacetic acid; Gradient: a 0-minute hold at 2400 B, 24-64% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.5 mg, and its estimated purity by LCMS analysis was 92.5%. Analysis condition B: Retention time=1.82 min; ESI-MS(+) m/z [M+3H]³⁺: 741.2.

### Preparation of Example 1304

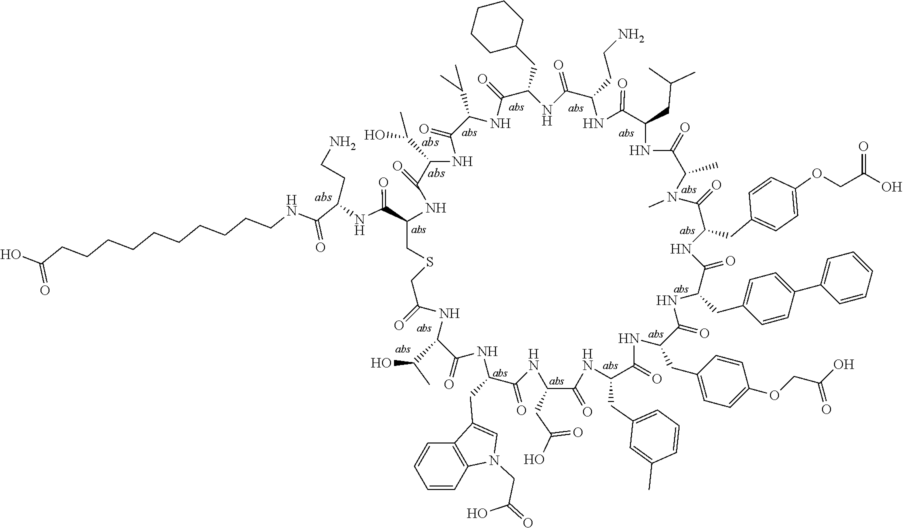 [SMILES: Cc1cccc(C[C@@H]2NC(=O)[C@H](CC(=O)O)NC(=O)[C@H](Cc3cn(CC(=O)O)c4ccccc34)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1304 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile:water with 0.1% trifluoroacetic acid; Gradient: a 0-minute hold at 27% B, 27-67% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.5 mg, and its estimated purity by LCMS analysis was 93.9%. Analysis condition A: Retention time=1.69 min; ESI-MS(+) m/z [M+2H]²⁺: 1192.1.

### Preparation of Example 1305

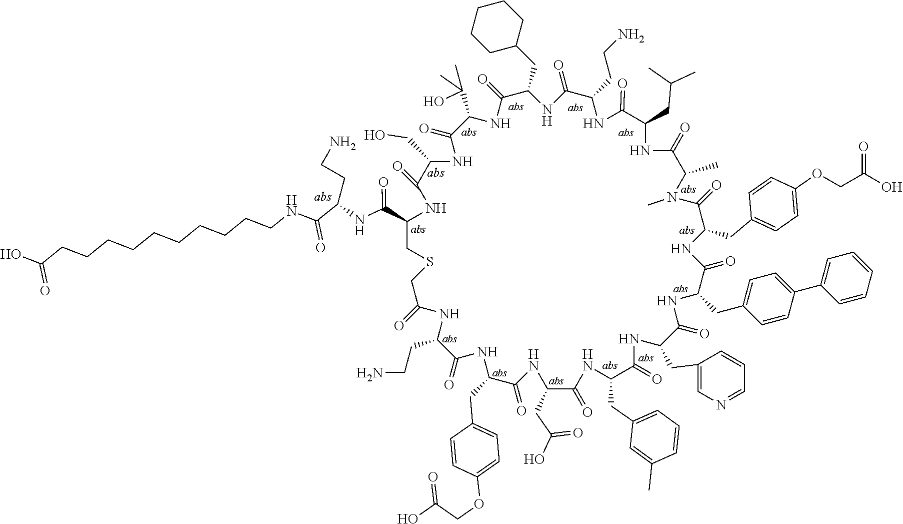 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccnc3)NC2=O)c1]

Example 1305 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Ala(3-pyridyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 19% B, 19-59% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 95.9%. Analysis condition A: Retention time=1.66 min; ESI-MS(+) m/z [M+2H]²⁺: 1145.1.

### Preparation of Example 1306

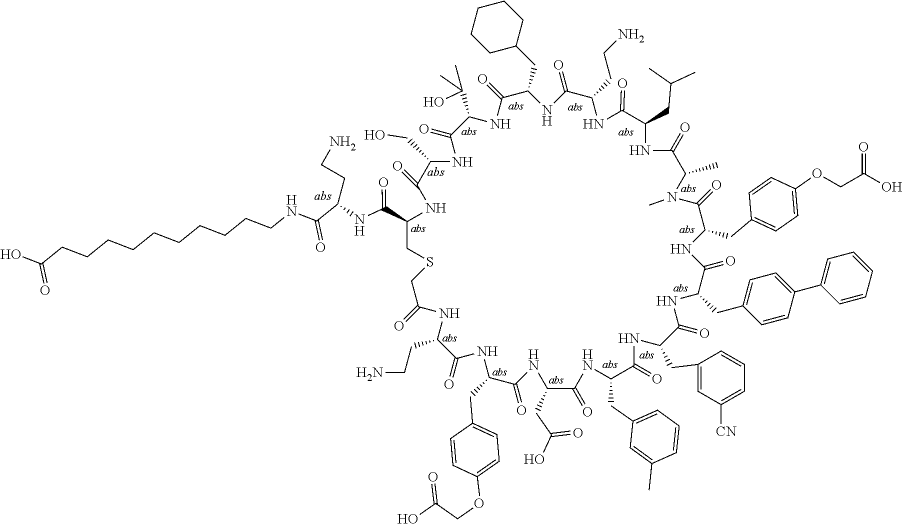 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(C#N)c3)NC2=O)c1]

Example 1306 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(3-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 20% B, 20-60% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.4 mg, and its estimated purity by LCMS analysis was 91.2%. Analysis condition A: Retention time=1.69 min; ESI-MS(+) m/z [M+2H]²⁺: 1157.2.

### Preparation of Example 1307

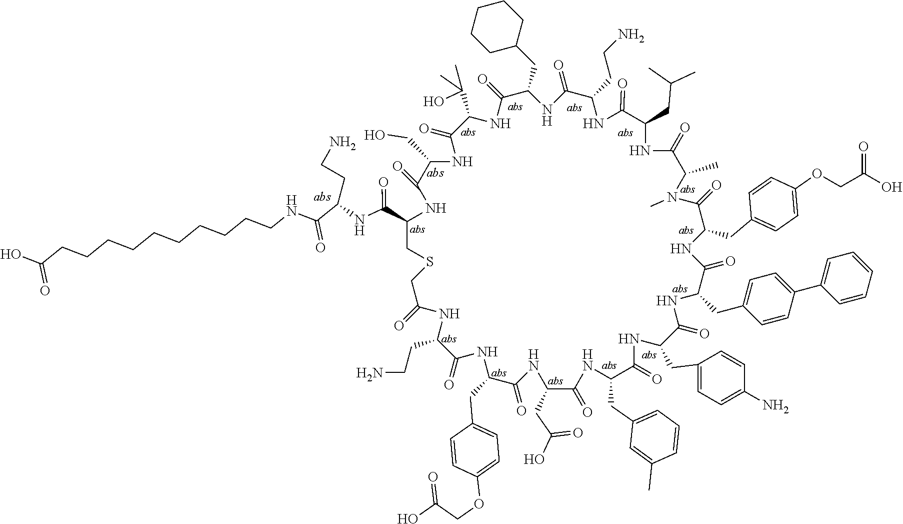 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(N)cc3)NC2=O)c1]

Example 1307 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-NHBoc)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 19% B, 19-59% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.1 mg, and its estimated purity by LCMS analysis was 98.2%. Analysis condition A: Retention time=1.67 min; ESI-MS(+) m/z [M+2H]²⁺: 1152.1.

### Preparation of Example 1308

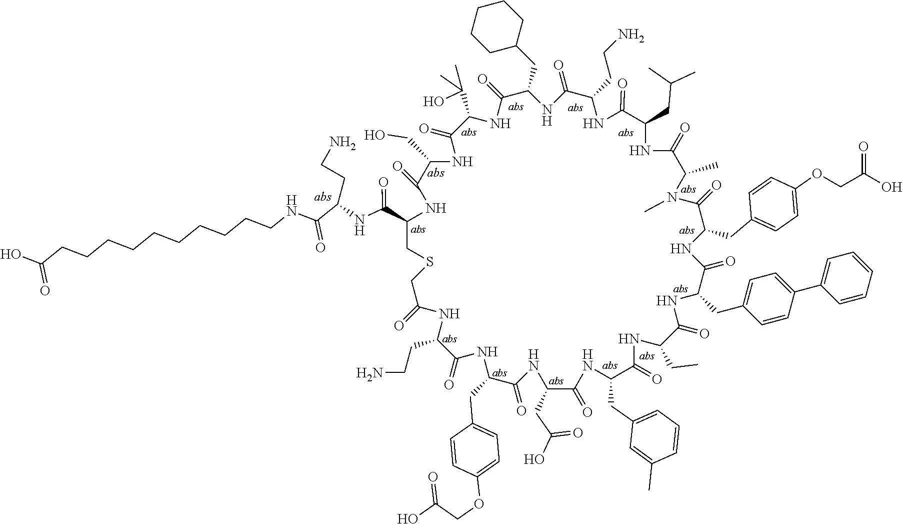 [SMILES: CC[C@@H]1NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC1=O]

Example 1308 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Abu-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05%0 trifluoroacetic acid; Mobile Phase B: 95:5 acetonitrile:water with 0.05%˜trifluoroacetic acid; Gradient: a 0-minute hold at 2200 B, 22-62% B over 25 minutes, then a 0-minute hold at 100% a B; Flow Rate: 20 mL/min; Column Temperature: 25 C. Fraction collection was triggered by MS signals. Fractions containing the desired product were combined and dried via centrifugal evaporation. The yield of the product was 26.2 mg, and its estimated purity by LCMS analysis was 93.8%. Analysis condition A: Retention time=1.73 min; ESI-MS(+) m/z [M+3H]³⁺: 742.3.

### Preparation of Example 1309

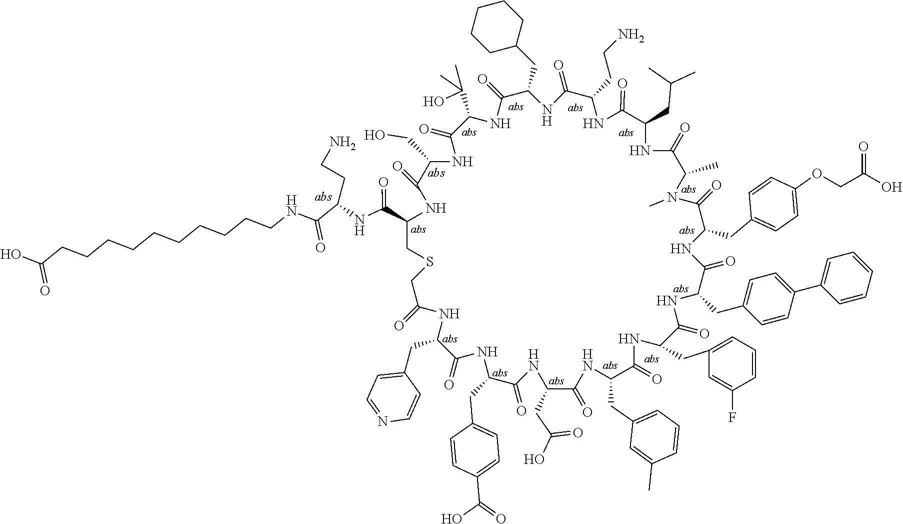 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Example 1309 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Phe(3-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 19% B, 19-59% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.1 mg, and its estimated purity by LCMS analysis was 96.4%. Analysis condition B: Retention time=1.91 min; ESI-MS(+) m/z [M+2H]²⁺: 1163.2.

### Preparation of Example 1310

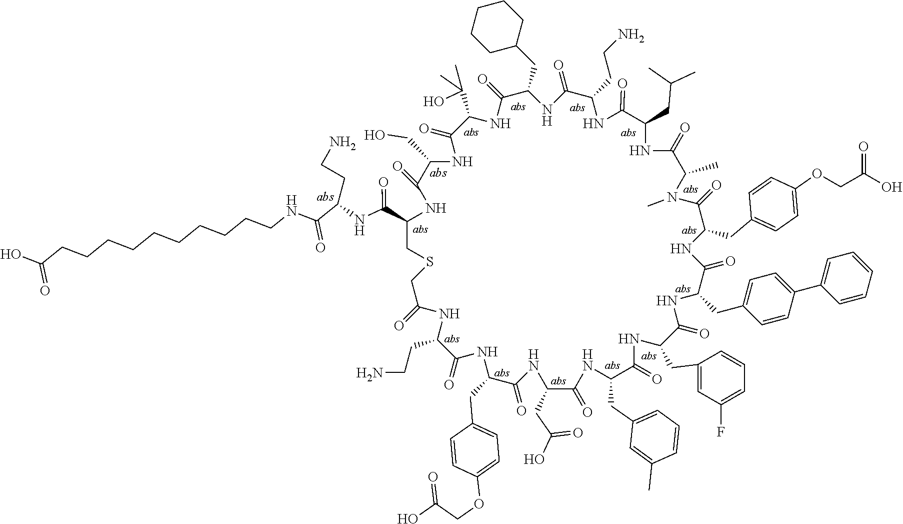 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Example 1310 was prepared, using 2-Chlorotrityl resin pre-loaded with 11-((((9H-fluoren-9-yl)methoxy)carbonyl)amino)undecanoic acid on a 30 μmol scale, following the general synthetic sequence described for the preparation of Example 1002 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed Fmoc-Phe(3-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile:water with 0.05% trifluoroacetic acid; Gradient: a 0-minute hold at 20% B, 20-58% B over 28 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.5 mg, and its estimated purity by LCMS analysis was 96%. Analysis condition A: Retention time=1.77 min; ESI-MS(+) m/z [M+3H]³⁺: 769.

### Preparation of Example 1311

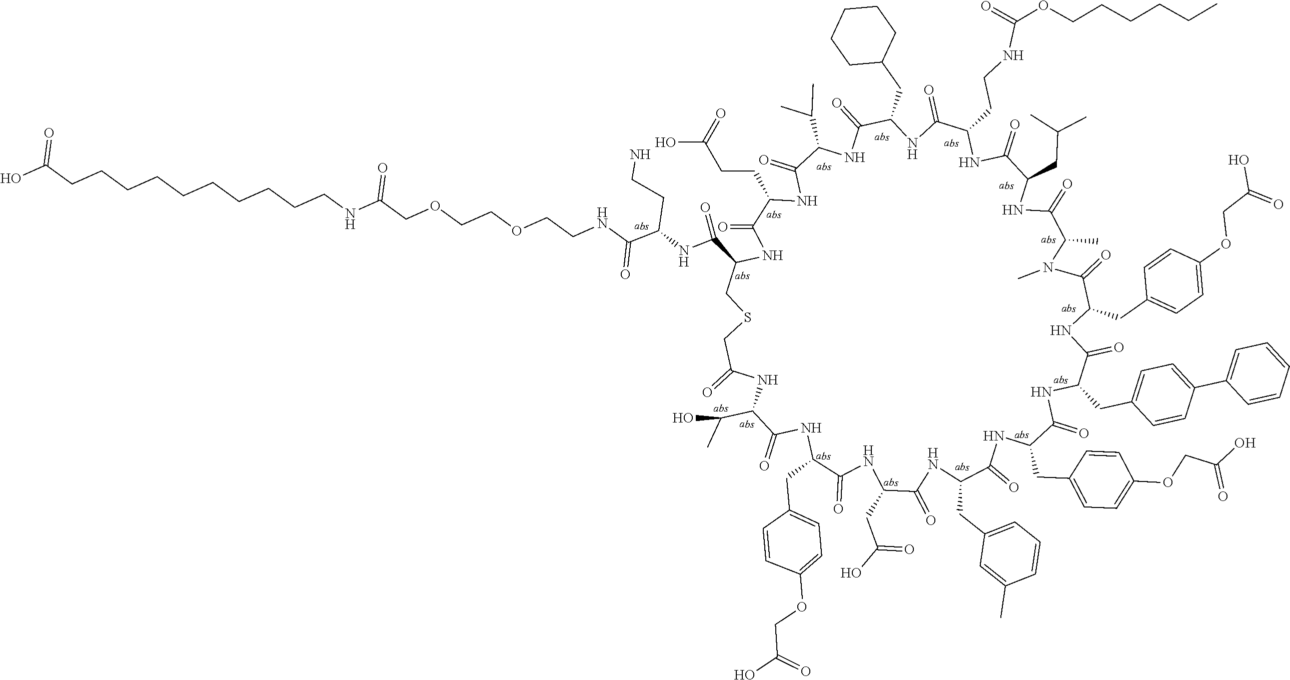 [SMILES: CCCCCCOC(=O)NCC[C@@H]1NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC1=O]

Product from Example 1310 (20 mg, 7.89 μmol) was dissolved in DMF (0.3 mL) and shaken for 1 min. To the solution was added the above freshly made 0.1 mL DMF solution of N-ethyl-N-isopropylpropan-2-amine (0.018 mL, 0.101 mmol) followed by the addition of 0.1 mL DMF solution of hexyl carbonochloridate (1.658 mg, 10.07 μmol). The resulting clear colorless solution was shaken at rt for 3 h. The reaction was concentrated and submitted for purification. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 35% B, 35-90% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 2 mg, and its estimated purity by LCMS analysis was 93.6%. Analysis condition B: Retention time=2.17 min; ESI-MS(+) m/z [M+2H]²⁺: 1332.3.

### Preparation of Example 1312

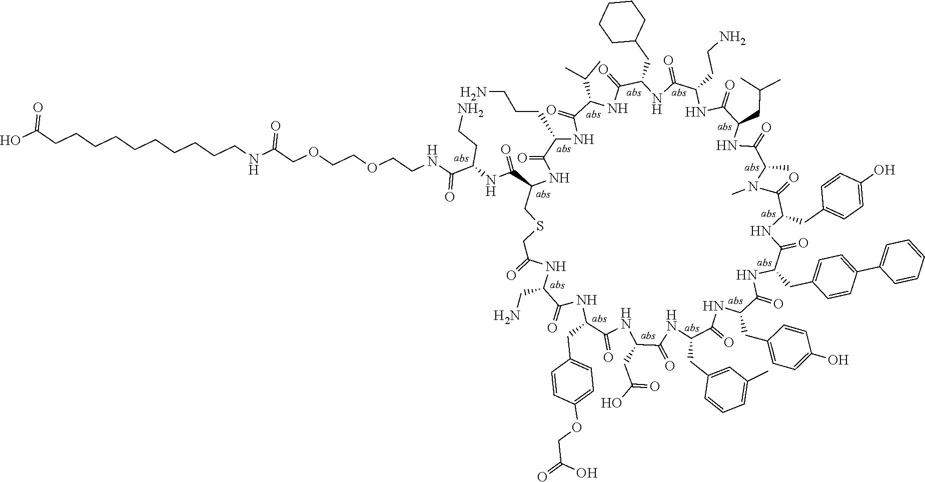 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1312 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 26.4 mg, and its estimated purity by LCMS analysis was 94.8%. Analysis condition B: Retention time=1.68 min; ESI-MS(+) m/z [M+2H]²⁺: 1194.2.

### Preparation of Example 1313

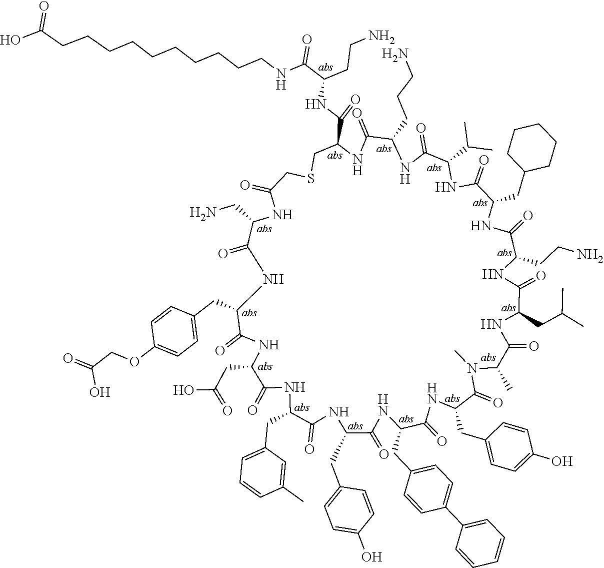 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1313 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 14.4 mg, and its estimated purity by LCMS analysis was 100%. Analysis condition A: Retention time=1.65 min; ESI-MS(+) m/z [M+2H]²⁺: 1122.3.

### Preparation of Example 1314

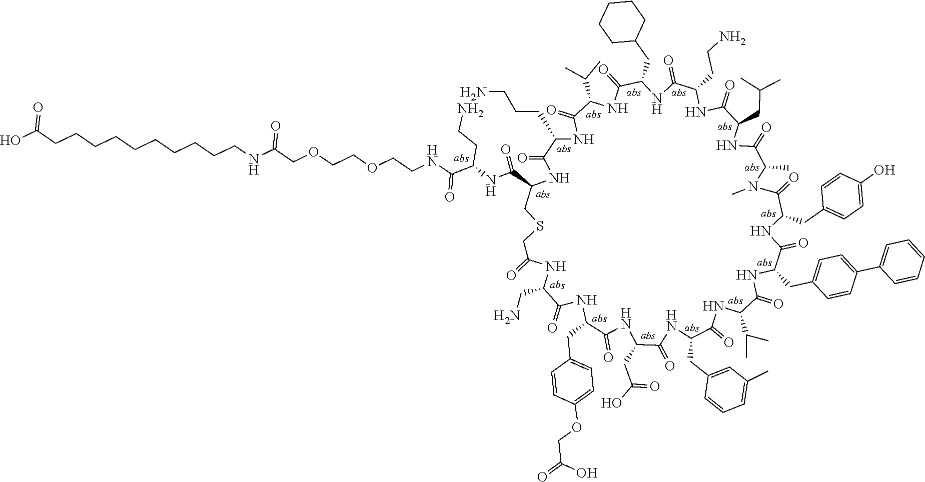 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1314 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 21 mg, and its estimated purity by LCMS analysis was 94.3%. Analysis condition B: Retention time=1.67 min; ESI-MS(+) m/z [M+3H]²⁺: 775.1.

### Preparation of Example 1315

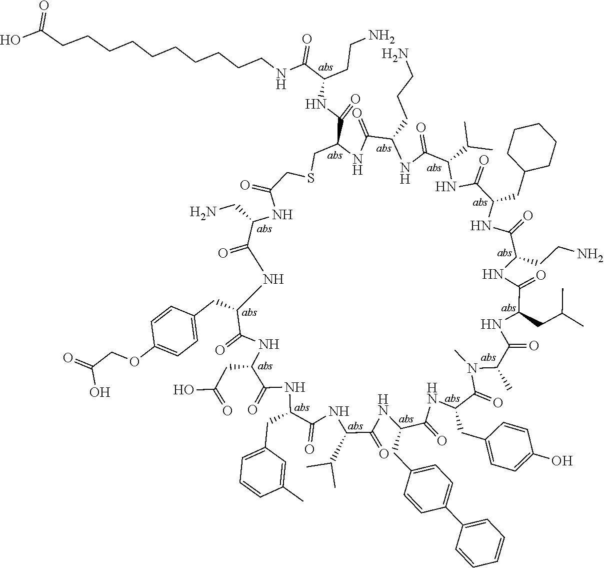 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1315 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: a 0-minute hold at 18% B, 18-58% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 19.1 mg, and its estimated purity by LCMS analysis was 100%. Analysis condition A: Retention time=1.7 min; ESI-MS(+) m/z [M+2H]²⁺: 1090.

### Preparation of Example 1316

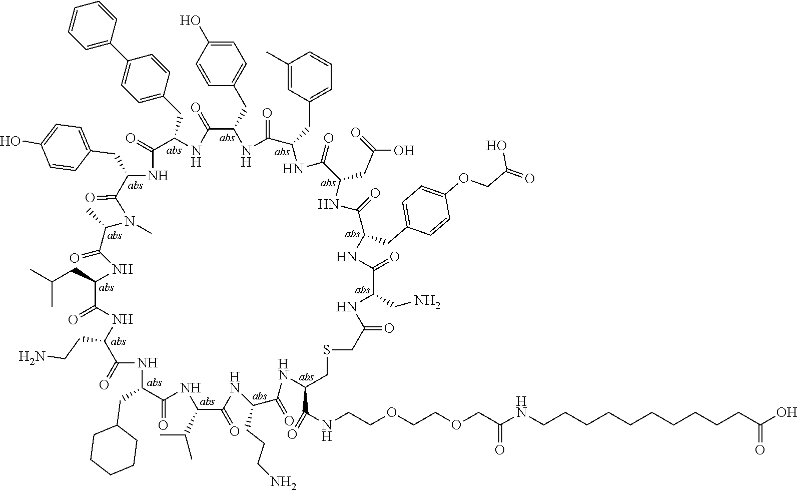 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1316 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 56.5 mg, and its estimated purity by LCMS analysis was 94.8%. Analysis condition A: Retention time=1.69 min; ESI-MS(+) m/z [M+2H]²⁺: 1144.2.

### Preparation of Example 1317

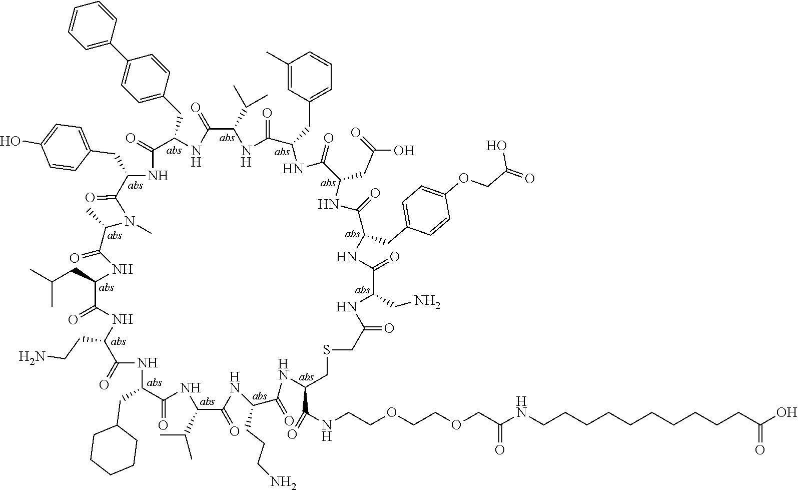 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1317 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 21% B, 21-61% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 39.6 mg, and its estimated purity by LCMS analysis was 92%. Analysis condition B: Retention time=1.75 min; ESI-MS(+) m/z [M+2H]²⁺: 1112.3.

### Preparation of Example 1318

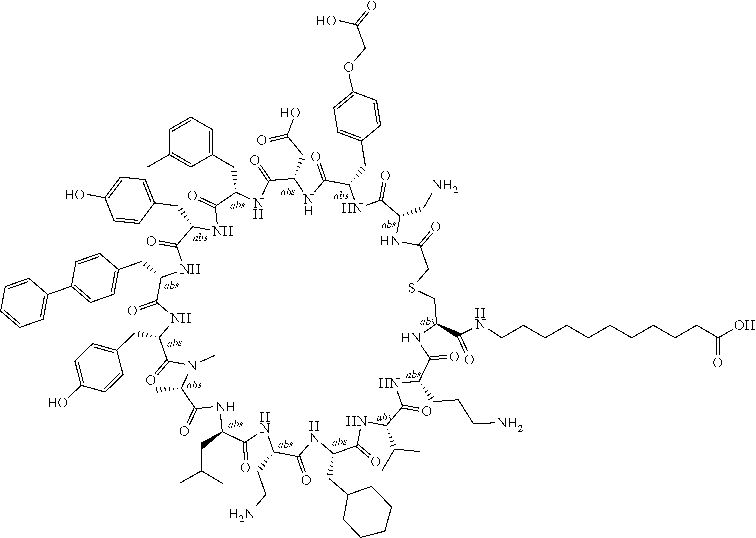 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 1318 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 20.3 mg, and its estimated purity by LCMS analysis was 91%. Analysis condition B: Retention time=1.61 min; ESI-MS(+) m/z [M+3H]²⁺: 715.1.

### Preparation of Example 1319

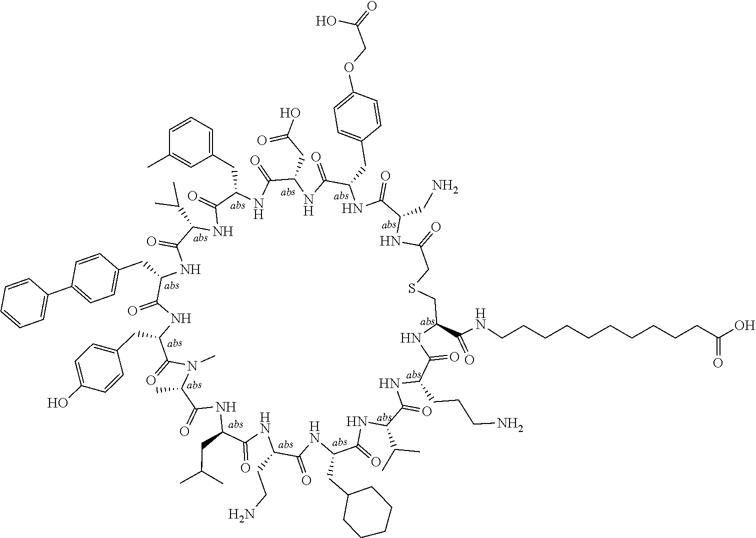 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1319 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 25.7 mg, and its estimated purity by LCMS analysis was 94.4%. Analysis condition B: Retention time=1.73 min; ESI-MS(+) m/z [M+2H]²⁺: 1039.5.

### Preparation of Example 1320

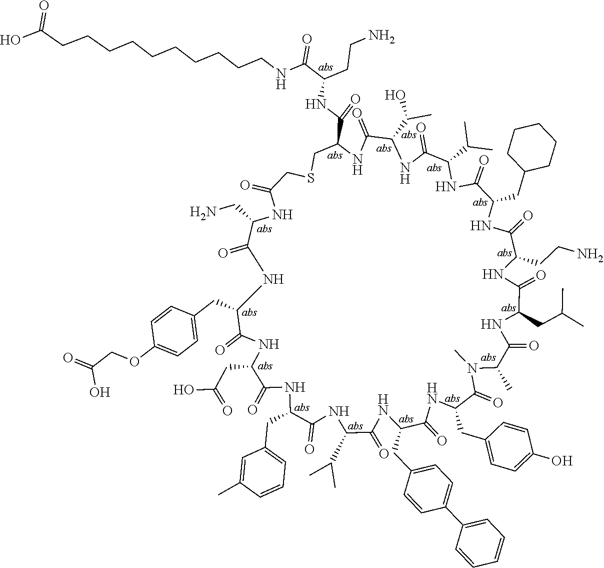 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1320 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 8.8 mg, and its estimated purity by LCMS analysis was 95.9%. Analysis condition A: Retention time=1.7 min; ESI-MS(+) m/z [M+2H]²⁺: 1083.2.

### Preparation of Example 1321

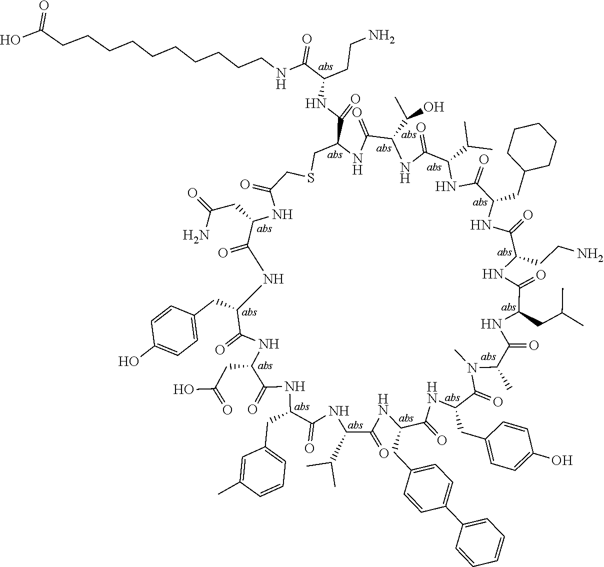 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1321 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 26% B, 26-66% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile: water with 0.1% trifluoroacetic acid; Gradient: a 0-minute hold at 26% B, 26-66% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 3.1 mg, and its estimated purity by LCMS analysis was 100%. Analysis condition A: Retention time=1.74 min; ESI-MS(+) m/z [M+2H]²⁺: 1068.9.

### Preparation of Example 1322

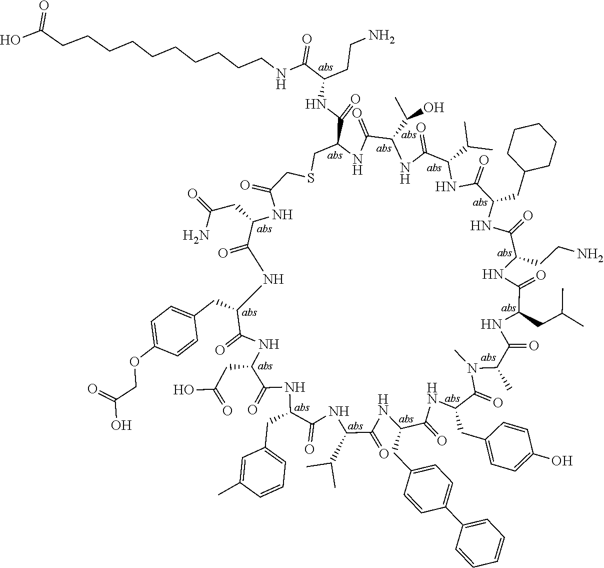 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1322 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 22% B, 22-62% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.7 mg, and its estimated purity by LCMS analysis was 100%. Analysis condition A: Retention time=1.67 min; ESI-MS(+) m/z [M+2H]²⁺: 1097.2.

### Preparation of Example 1323

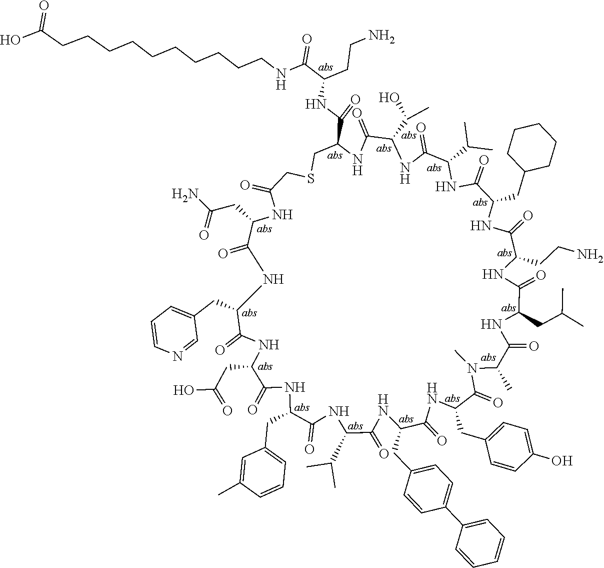 [SMILES: Cc1cccc(C[C@@H]2NC(=O)[C@H](CC(=O)O)NC(=O)[C@H](Cc3cccnc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 1323 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile: water with 0.1% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2 mg, and its estimated purity by LCMS analysis was 100%. Analysis condition A: Retention time=1.88 min; ESI-MS(+) m/z [M+2H]²⁺: 1061.9.

### Preparation of Example 1324

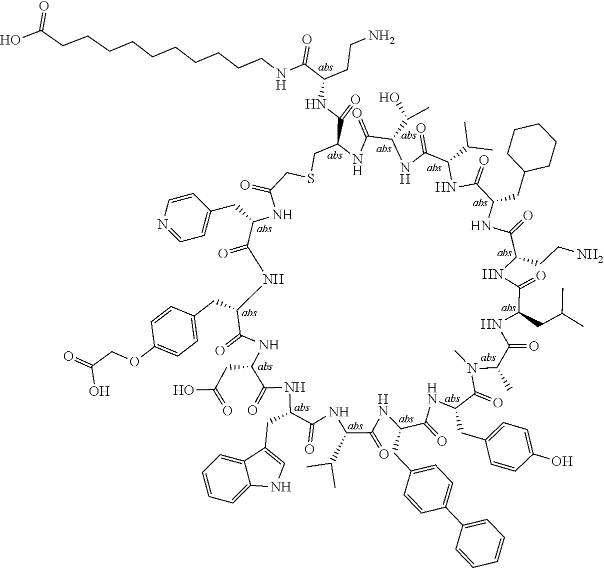 [SMILES: CC(C)C[C@H]1NC(=O)[C@H](C)N(C)C(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1324 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 17.4 mg, and its estimated purity by LCMS analysis was 96.5%. Analysis condition A: Retention time=1.62 min; ESI-MS(+) m/z [M+2H]²⁺: 1127.2.

### Preparation of Example 1325

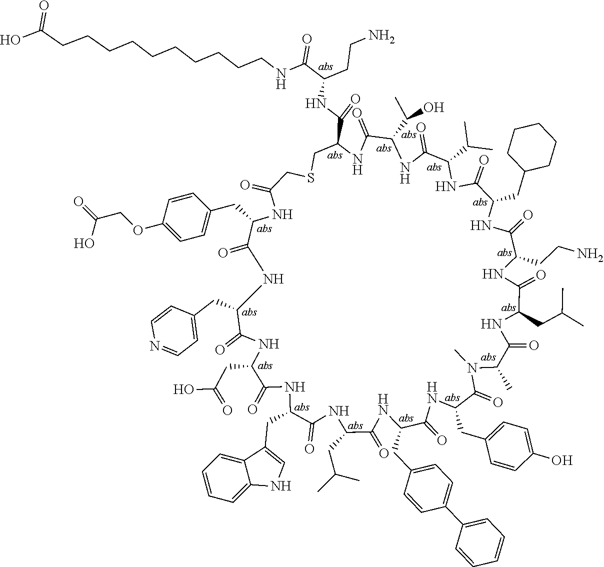 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(O)cc2)NC(=O)[C@H](Cc2ccc(-c3ccccc3)cc2)NC1=O]

Example 1325 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-50% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 27% B, 27-67% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 14.4 mg, and its estimated purity by LCMS analysis was 94.8%. Analysis condition B: Retention time=1.74 min; ESI-MS(+) m/z [M+2H]²⁺: 1133.8.

### Preparation of Example 1326

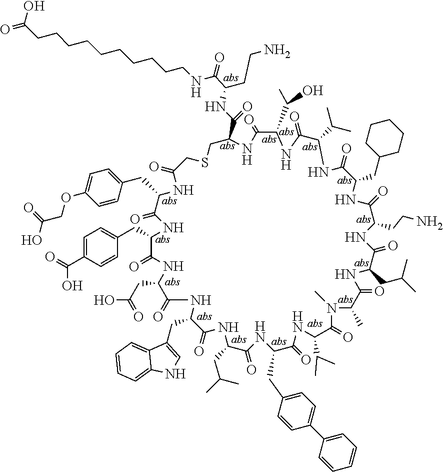 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1326 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 19% B, 19-59% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10.2 mg, and its estimated purity by LCMS analysis was 100%. Analysis condition A: Retention time=1.73 min; ESI-MS(+) m/z [M+2H]²⁺: 1124.

### Preparation of Example 1327

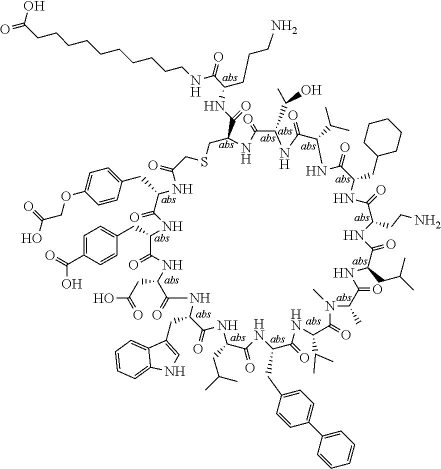 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1327 was prepared, using 2-Chlorotrityl resin pre-loaded with 11-((((9H-fluoren-9-yl)methoxy)carbonyl)amino)undecanoic acid on a 50 □mol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 14.1 mg, and its estimated purity by LCMS analysis was 96.4%. Analysis condition A: Retention time=1.71 min; ESI-MS(+) m/z [M+2H]²⁺: 1128.2.

### Preparation of Example 1328

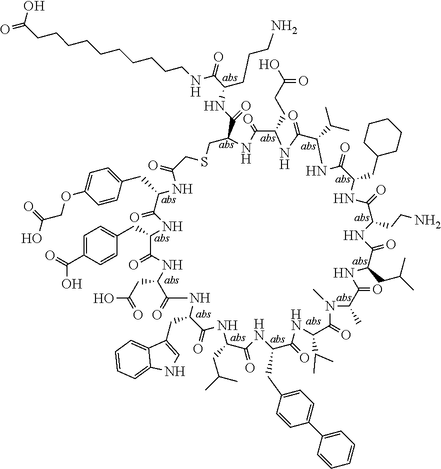 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1328 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 24% B, 24-64% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 40.1 mg, and its estimated purity by LCMS analysis was 91.2%. Analysis condition B: Retention time=1.82 min; ESI-MS(+) m/z [M+2H]²⁺: 1145.1.

### Preparation of Example 1329

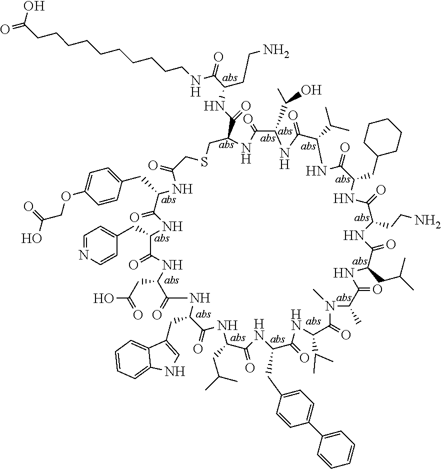 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1329 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 24% B, 24-64% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 38.2 mg, and its estimated purity by LCMS analysis was 95.7%. Analysis condition B: Retention time=1.74 min; ESI-MS(+) m/z [M+2H]²⁺: 1102.1.

### Preparation of Example 1330

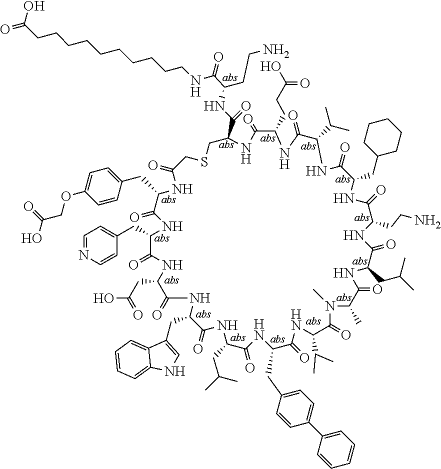 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1330 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-pm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 13.6 mg, and its estimated purity by LCMS analysis was 97.7%. Analysis condition A: Retention time=1.56 min; ESI-MS(+) m/z [M+2H]²⁺: 1116.

### Preparation of Example 1331

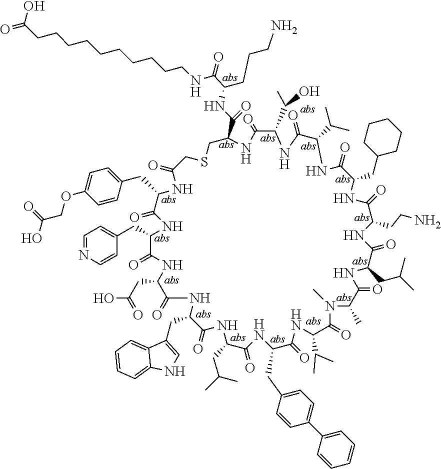 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1331 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-64% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 41.1 mg, and its estimated purity by LCMS analysis was 95.5%. Analysis condition A: Retention time=1.69 min; ESI-MS(+) m/z [M+2H]²⁺: 1108.9.

### Preparation of Example 1332

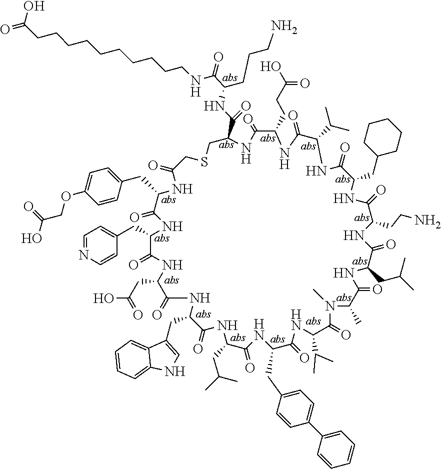 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1332 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 22 mg, and its estimated purity by LCMS analysis was 94.7%. Analysis condition B: Retention time=1.72 min; ESI-MS(+) m/z [M+2H]²⁺: 1122.8.

### Preparation of Example 1333

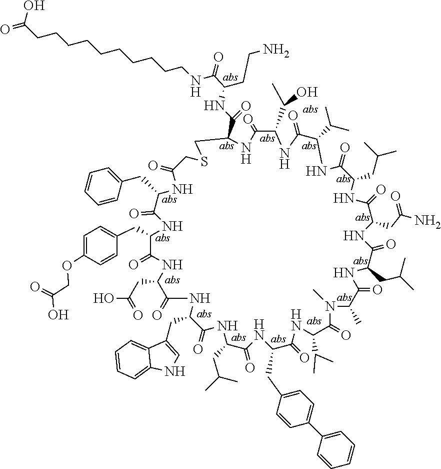 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1333 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60%0 B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 37.4 mg, and its estimated purity by LCMS analysis was 98.30%. Analysis condition A: Retention time=1.73 mi; ESI-MS(+) m/z [M+2H]²⁺: 1087.2.

### Preparation of Example 1334

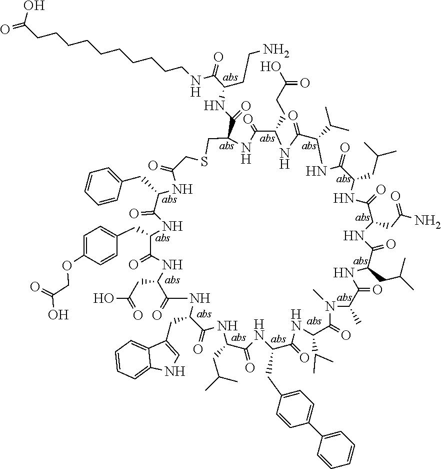 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1334 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-pm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 28% B, 28-68% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 11.7 mg, and its estimated purity by LCMS analysis was 95.2%. Analysis condition B: Retention time=2.04 min; ESI-MS(+) m/z [M+2H]²⁺: 1102.3.

### Preparation of Example 1335

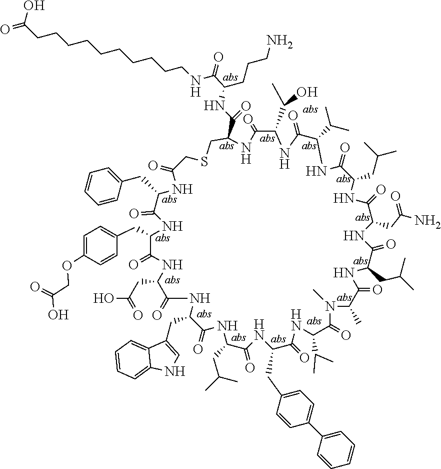 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1335 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.100 trifluoroacetic acid; Mobile Phase B: 95:5 acetonitrile:water with 0.1% trifluoroacetic acid; Gradient: a 0-minute hold at 32% B, 32-72% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 13.2 mg, and its estimated purity by LCMS analysis was 95.8%. Analysis condition A: Retention time=1.57 min; ESI-MS(+) m/z [M+2H]²⁺: 1096.

### Preparation of Example 1336

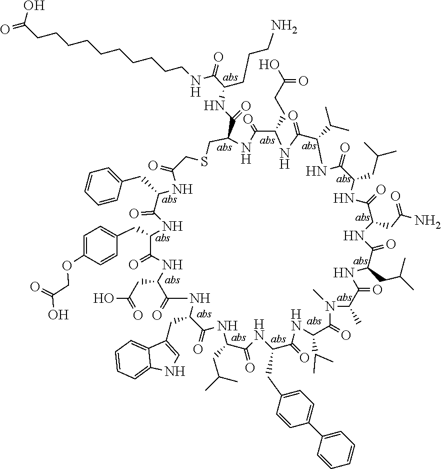 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1336 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 12.9 mg, and its estimated purity by LCMS analysis was 92%. Analysis condition A: Retention time=1.62 min; ESI-MS(+) m/z [M+2H]²⁺: 1110.1.

### Preparation of Example 1337

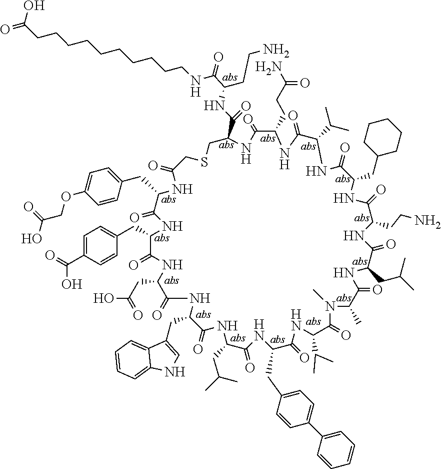 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1337 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A” “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 21% B. 21-61% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.5 mg, and its estimated purity by LCMS analysis was 97.7%. Analysis condition B: Retention time=1.91 min; ESI-MS(+) m/z [M+2H]²⁺: 1137.1.

### Preparation of Example 1338

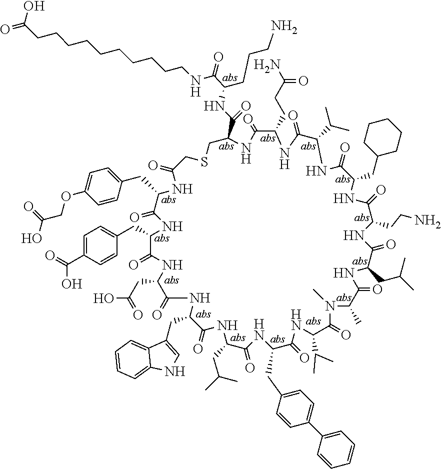 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1338 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 36.6 mg, and its estimated purity by LCMS analysis was 97.6%. Analysis condition B: Retention time=1.89 min; ESI-MS(+) m/z [M+2H]²⁺: 1144.1.

### Preparation of Example 1339

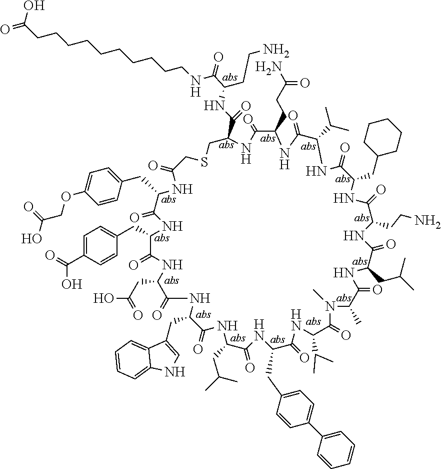 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1339 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 23% B, 23-63% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 42 mg, and its estimated purity by LCMS analysis was 94.2%. Analysis condition B: Retention time=1.85 min; ESI-MS(+) m/z [M+2H]²⁺: 1136.8.

### Preparation of Example 1340

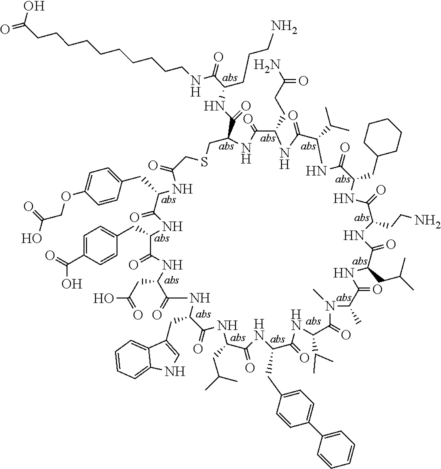 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1340 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 23% B, 23-63% B over 24 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 51.7 mg, and its estimated purity by LCMS analysis was 99.1%. Analysis condition B: Retention time=1.9 min; ESI-MS(+) m/z [M+2H]²⁺: 1144.1.

### Preparation of Example 1341

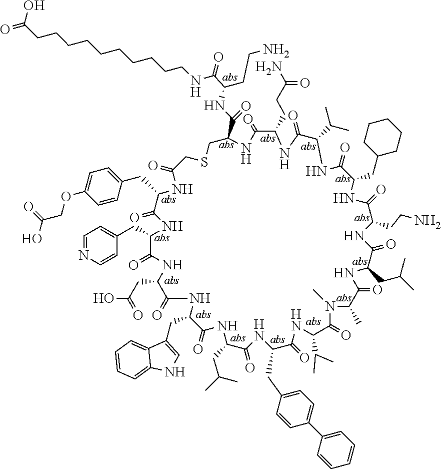 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1341 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.9 mg, and its estimated purity by LCMS analysis was 95.8%. Analysis condition A: Retention time=1.82 min; ESI-MS(+) m/z [M+2H]²⁺: 1115.3.

### Preparation of Example 1342

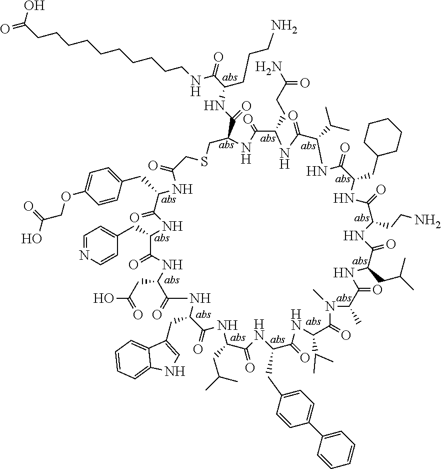 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1342 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 29.3 mg, and its estimated purity by LCMS analysis was 99.3%. Analysis condition B: Retention time=1.77 min; ESI-MS(+) m/z [M+2H]²⁺: 1122.3.

### Preparation of Example 1343

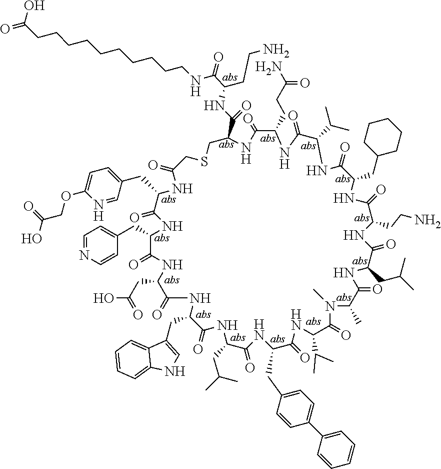 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)nc2)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1343 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 27% B, 27-67% B over 26 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 38.3 mg, and its estimated purity by LCMS analysis was 98.5%. Analysis condition B: Retention time=1.79 min; ESI-MS(+) m/z [M+3H]³⁺: 744.1.

### Preparation of Example 1344

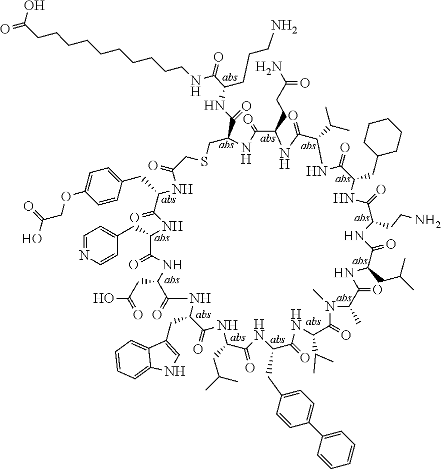 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1344 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 26% B, 26-66% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 41.5 mg, and its estimated purity by LCMS analysis was 99.4%. Analysis condition B: Retention time=1.78 min; ESI-MS(+) m/z [M+3H]³⁺: 749.1.

### Preparation of Example 1345

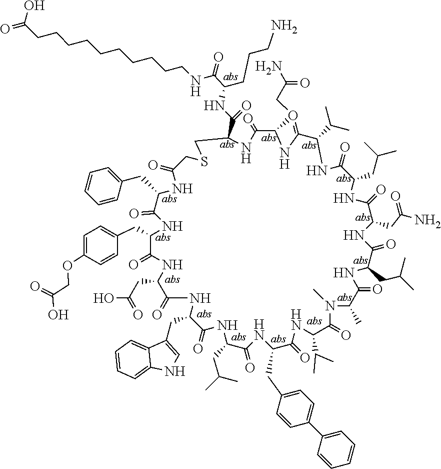

Example 1345 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 19% B, 19-59% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.2 mg, and its estimated purity by LCMS analysis was 93.9%. Analysis condition B: Retention time=2.01 min; ESI-MS(+) m/z [M+2H]²⁺: 1109.2.

### Preparation of Example 1346

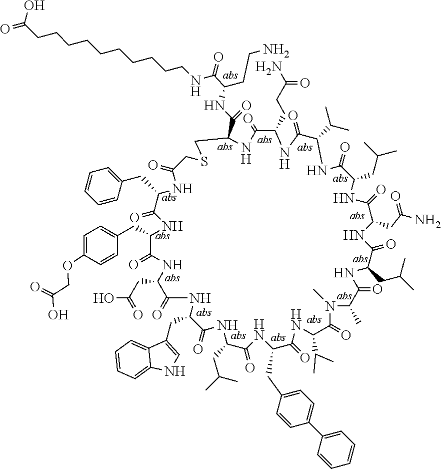 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1346 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 17% B, 17-57% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.3 mg, and its estimated purity by LCMS analysis was 95.3%. Analysis condition B: Retention time=2.05 min; ESI-MS(+) m/z [M+2H]²⁺: 1102.2.

### Preparation of Example 1347

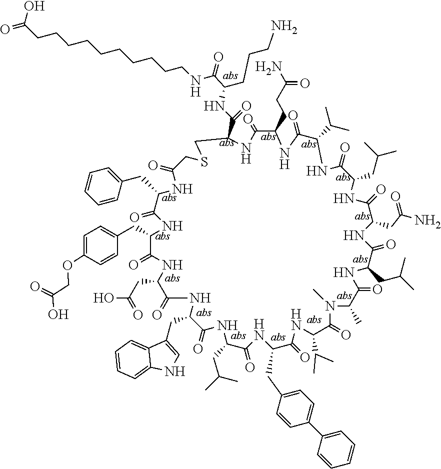 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1347 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 32% B, 32-72% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21 mg, and its estimated purity by LCMS analysis was 98.6%. Analysis condition A: Retention time=1.62 min; ESI-MS(+) m/z [M+2H]²⁺: 1109.2.

### Preparation of Example 1348

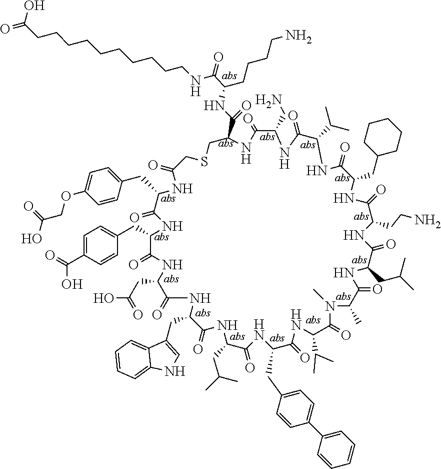 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1348 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.6 mg, and its estimated purity by LCMS analysis was 98.2%. Analysis condition B: Retention time=1.83 min; ESI-MS(+) m/z [M+2H]²⁺: 1130.1.

### Preparation of Example 1349

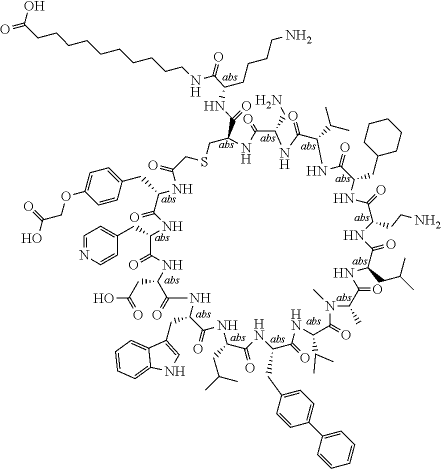 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1349 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.3 mg, and its estimated purity by LCMS analysis was 100%. Analysis condition A: Retention time=1.79 min; ESI-MS(+) m/z [M+3H]³⁺: 739.3.

### Preparation of Example 1350

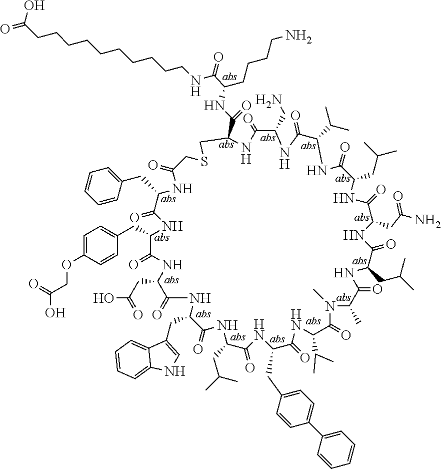 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1350 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 22% B, 22-62% B over 24 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 29.9 mg, and its estimated purity by LCMS analysis was 98.8%. Analysis condition B: Retention time=1.95 min; ESI-MS(+) m/z [M+2H]²⁺: 1095.2.

### Preparation of Example 1351

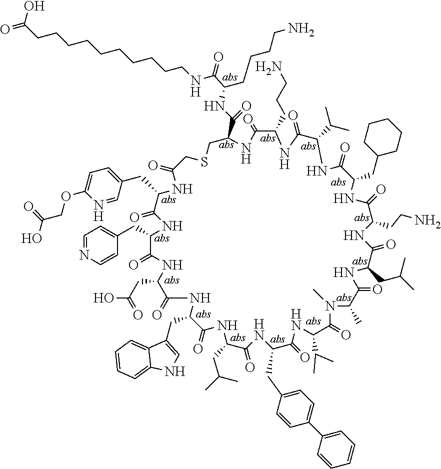 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)nc2)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1351 was prepared, using 2-Chlorotrityl resin pre-loaded with 11-((((9H-fluoren-9-yl)methoxy)carbonyl)amino)undecanoic acid on a 50 □mol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.8 mg, and its estimated purity by LCMS analysis was 86.7%. Analysis condition A: Retention time=1.74 min; ESI-MS(+) m/z [M+3H]³⁺: 748.3.

### Preparation of Example 1352

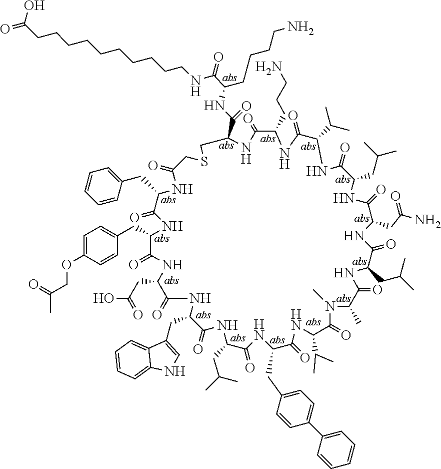 [SMILES: CC(=O)COc1ccc(C[C@@H]2NC(=O)[C@H](Cc3ccccc3)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC(=O)[C@H](Cc3c[nH]c4ccccc34)NC(=O)[C@H](CC(=O)O)NC2=O)cc1]

Example 1352 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.10% trifluoroacetic acid; Mobile Phase B: 95:5 acetonitrile:water with 0.1% trifluoroacetic acid; Gradient: a 0-minute hold at 29% B, 29-69% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.3 mg, and its estimated purity by LCMS analysis was 970%. Analysis condition A: Retention time=1.73 min; ESI-MS(+) m/z [M+2H]²⁺: 1109.2.

### Preparation of Example 1353

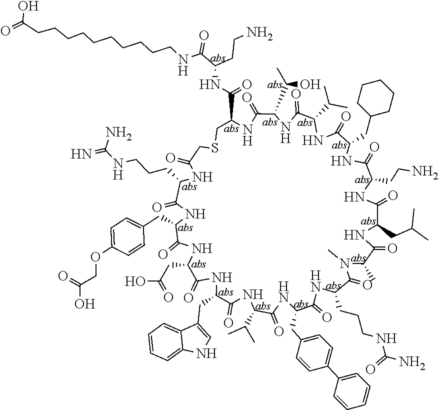 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1353 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.7 mg, and its estimated purity by LCMS analysis was 94.2%. Analysis condition A: Retention time=1.55 min; ESI-MS(+) m/z [M+2H]²⁺: 1128.

### Preparation of Example 1354

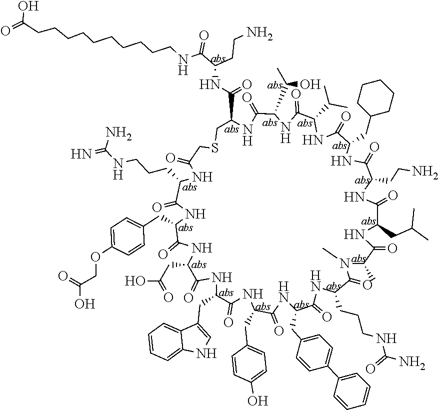 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1354 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.3 mg, and its estimated purity by LCMS analysis was 97.3%. Analysis condition A: Retention time=1.6 min; ESI-MS(+) m/z [M+2H]²⁺: 1160.

### Preparation of Example 1355

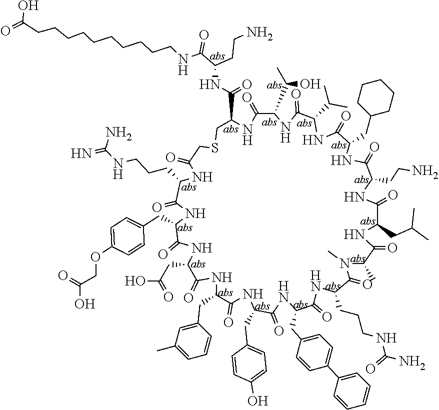 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(O)cc3)NC2=O)c1]

Example 1355 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33 mg, and its estimated purity by LCMS analysis was 92.6%. Analysis condition A: Retention time=1.55 min; ESI-MS(+) m/z [M+3H]³⁺: 765.1.

### Preparation of Example 1356

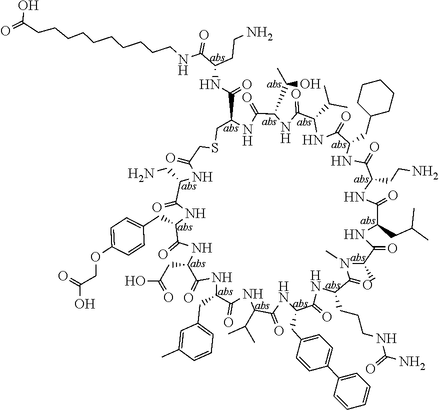 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)C(C(C)C)NC2=O)c1]

Example 1356 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 27% B, 27-67% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 28.7 mg, and its estimated purity by LCMS analysis was 94.7%. Analysis condition A: Retention time=1.64 min; ESI-MS(+) m/z [M+2H]²⁺: 1080.3.

### Preparation of Example 1357

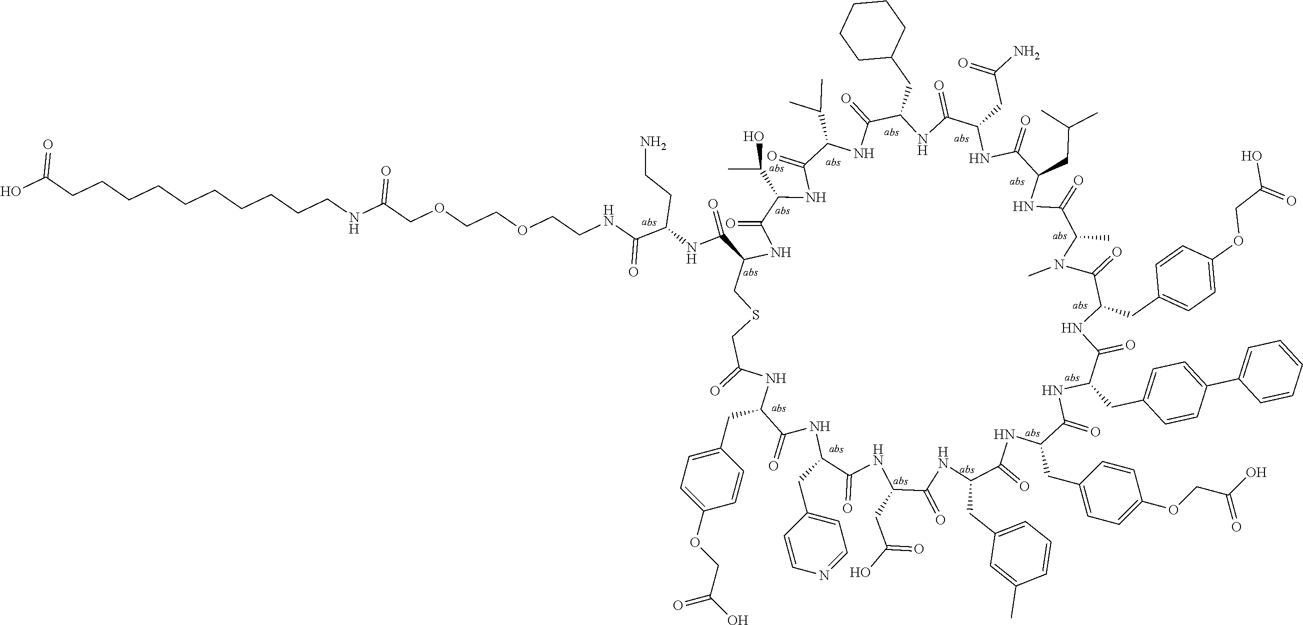 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1357 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.1 mg, and its estimated purity by LCMS analysis was 96.5%. Analysis condition B: Retention time=1.93 min; ESI-MS(+) m/z [M+2H]²⁺: 1283.9.

### Preparation of Example 1358

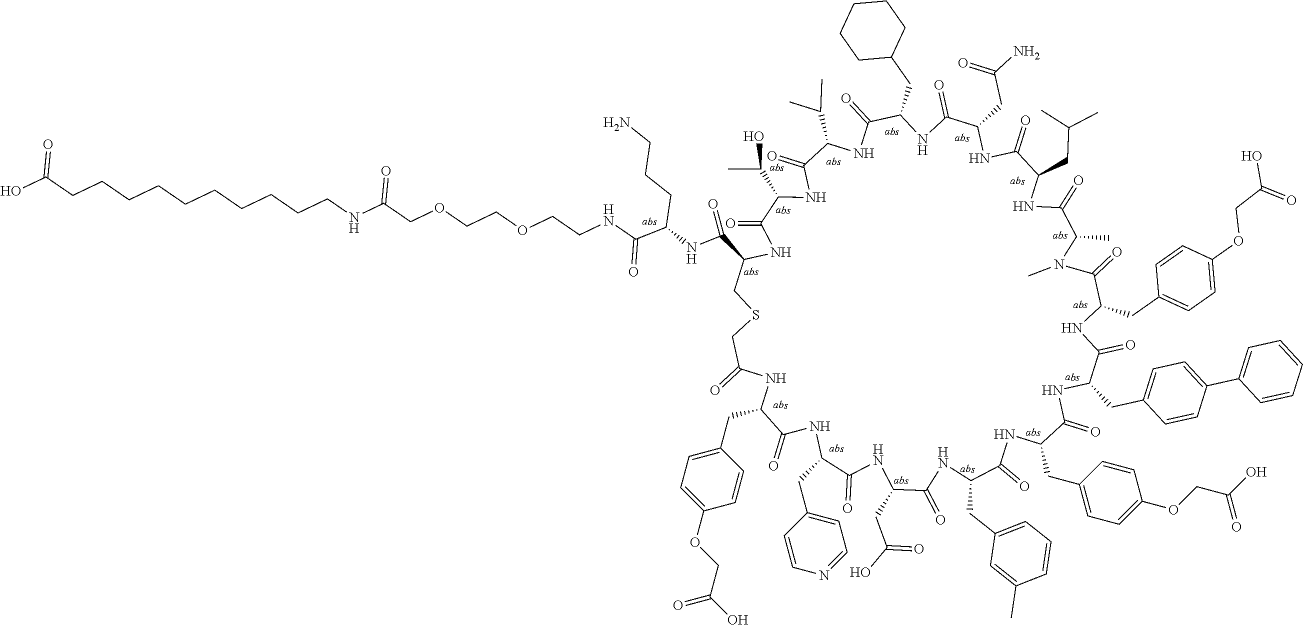 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1358 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 30% B, 30-70% B over 20 minutes, then a 0-minute hold at 100% B; Flow Rate: 20 mL/min; Column Temperature: 25 C. Fraction collection was triggered by UV signals. Fractions containing the desired product were combined and dried via centrifugal evaporation. The yield of the product was 19.1 mg, and its estimated purity by LCMS analysis was 100%. Analysis condition A: Retention time=1.46 min; ESI-MS(+) m/z [M+2H]²⁺: 1290.9.

### Preparation of Example 1359

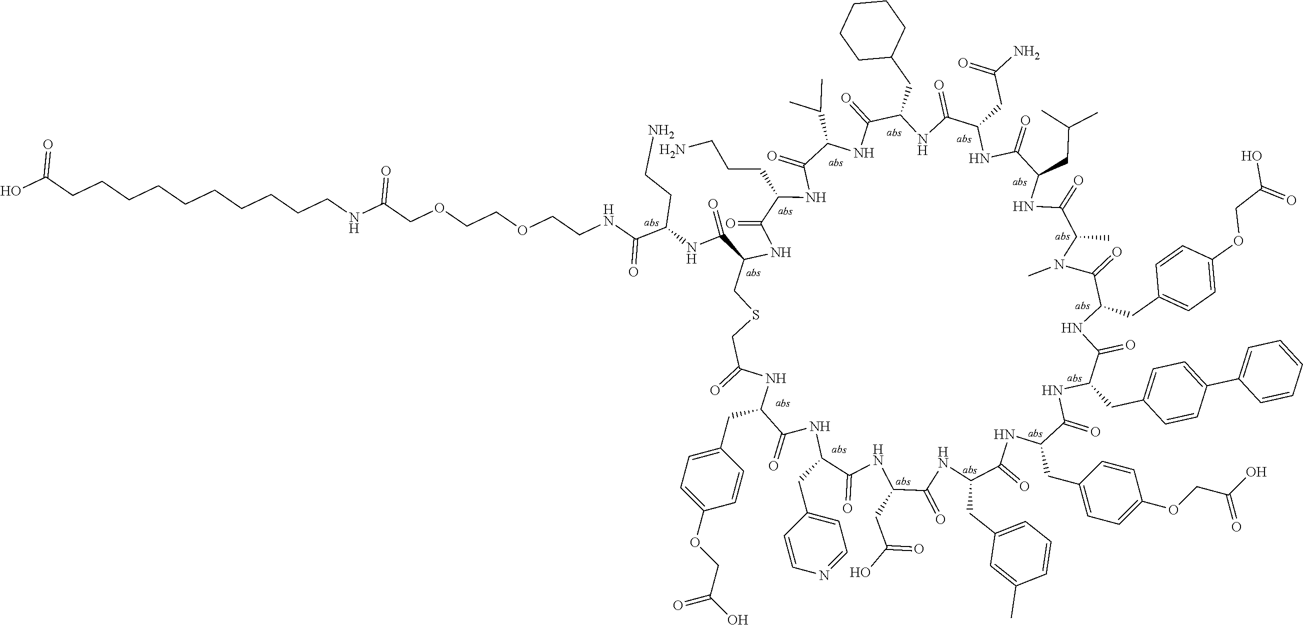 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1359 was prepared, using 2-Chlorotrityl resin pre-loaded with 11-((((9H-fluoren-9-yl)methoxy)carbonyl)amino)undecanoic acid on a 50 □mol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 32.2 mg, and its estimated purity by LCMS analysis was 85.8%. Analysis condition B: Retention time=1.82 min; ESI-MS(+) m/z [M+2H]²⁺: 1290.9.

### Preparation of Example 1360

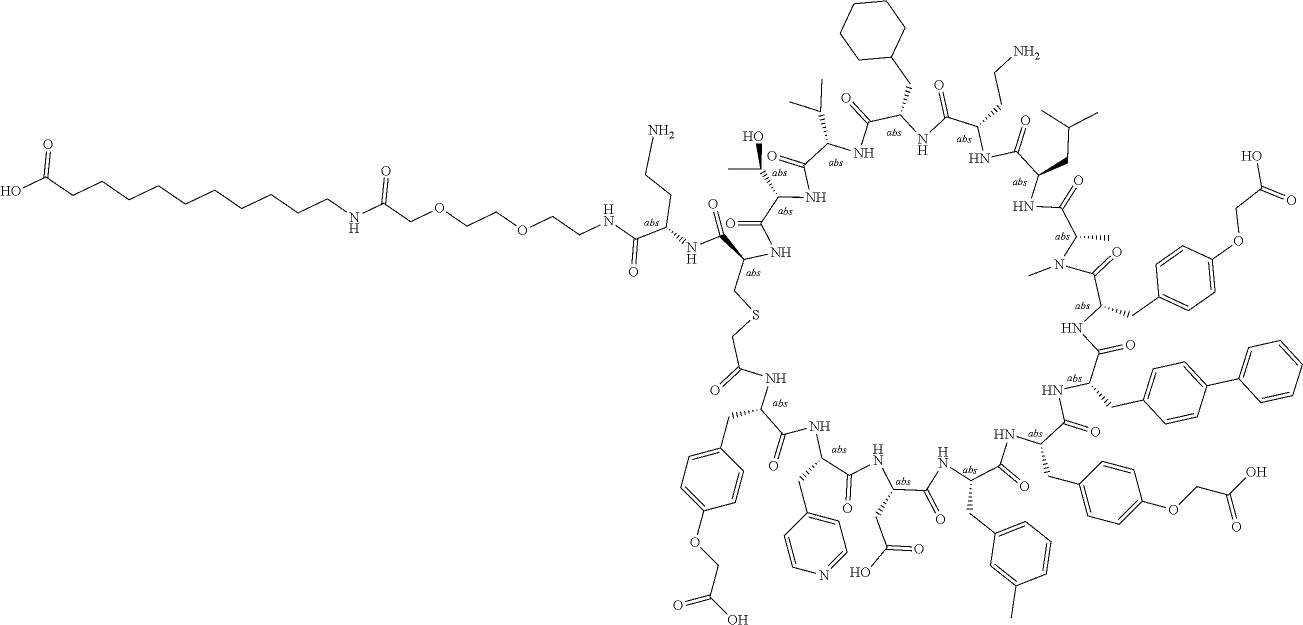 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1360 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 44.9 mg, and its estimated purity by LCMS analysis was 95%. Analysis condition B: Retention time=1.73 min; ESI-MS(+) m/z [M+2H]²⁺: 1276.9.

### Preparation of Example 1361

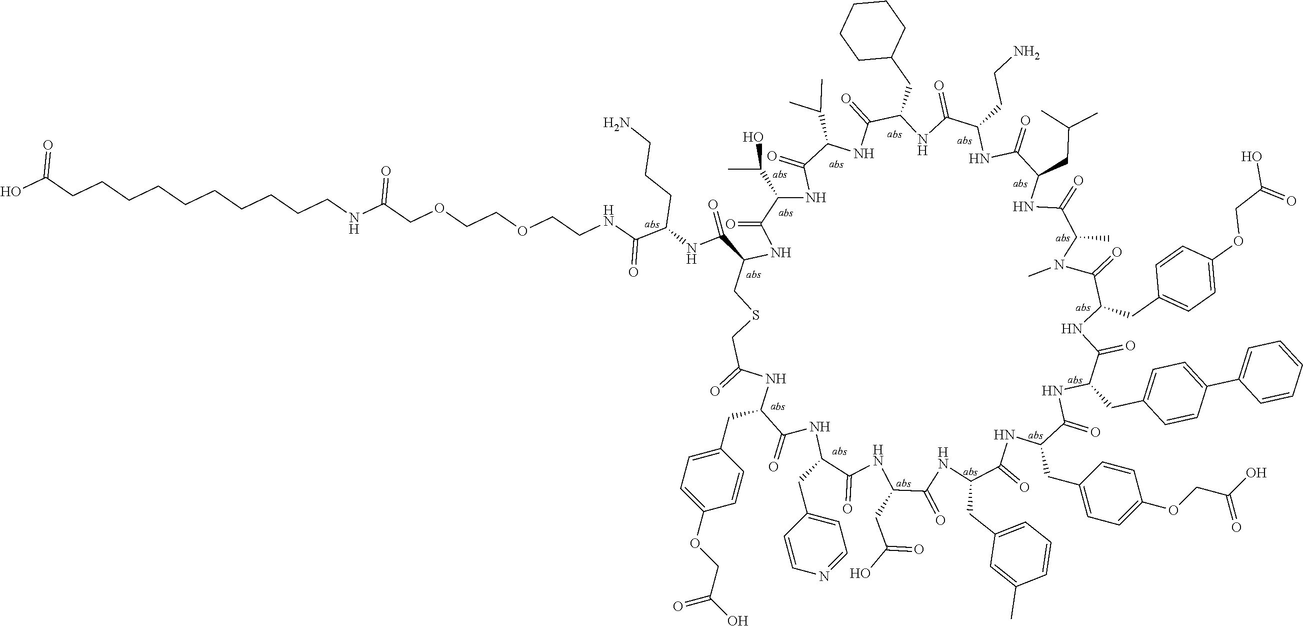 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1361 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 39.6 mg, and its estimated purity by LCMS analysis was 92.9%. Analysis condition B: Retention time=1.76 min; ESI-MS(+) m/z [M+2H]²⁺: 1283.9.

### Preparation of Example 1362

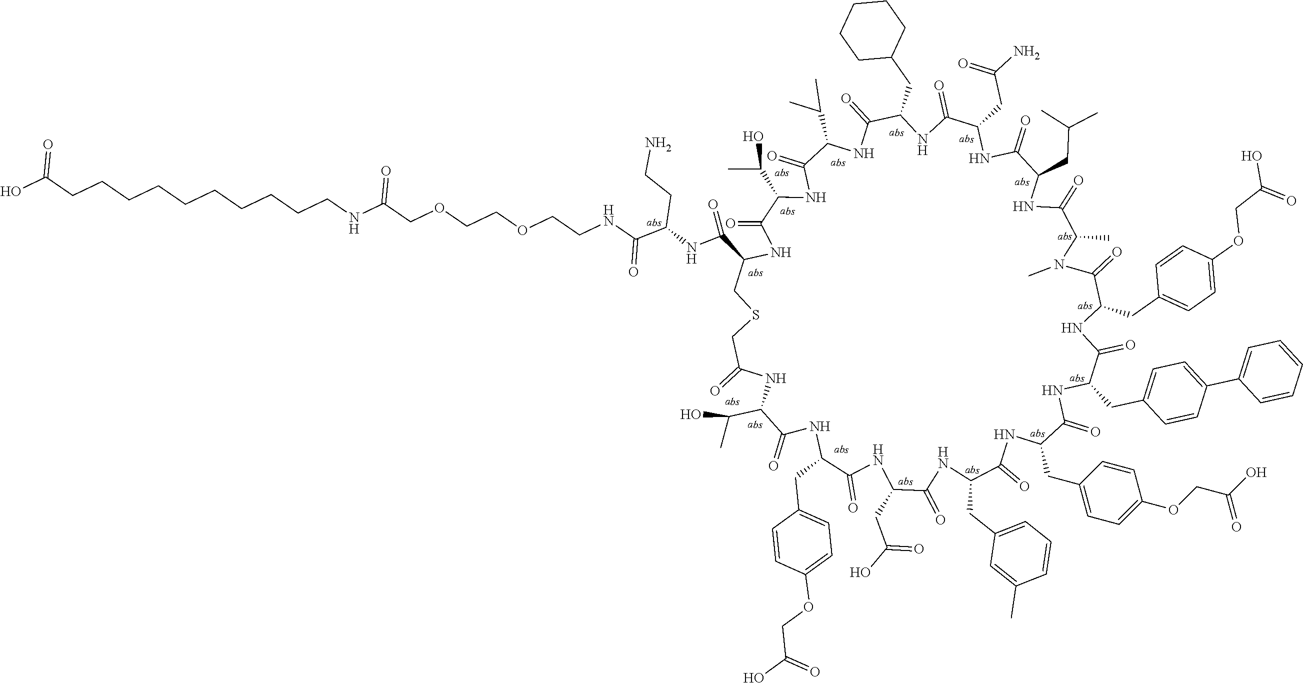 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1362 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 24.1 mg, and its estimated purity by LCMS analysis was 94.7%. Analysis condition B: Retention time=2.09, 2.12 min; ESI-MS(+) m/z [M+2H]²⁺: 1260.06, 1260.41.

### Preparation of Example 1363

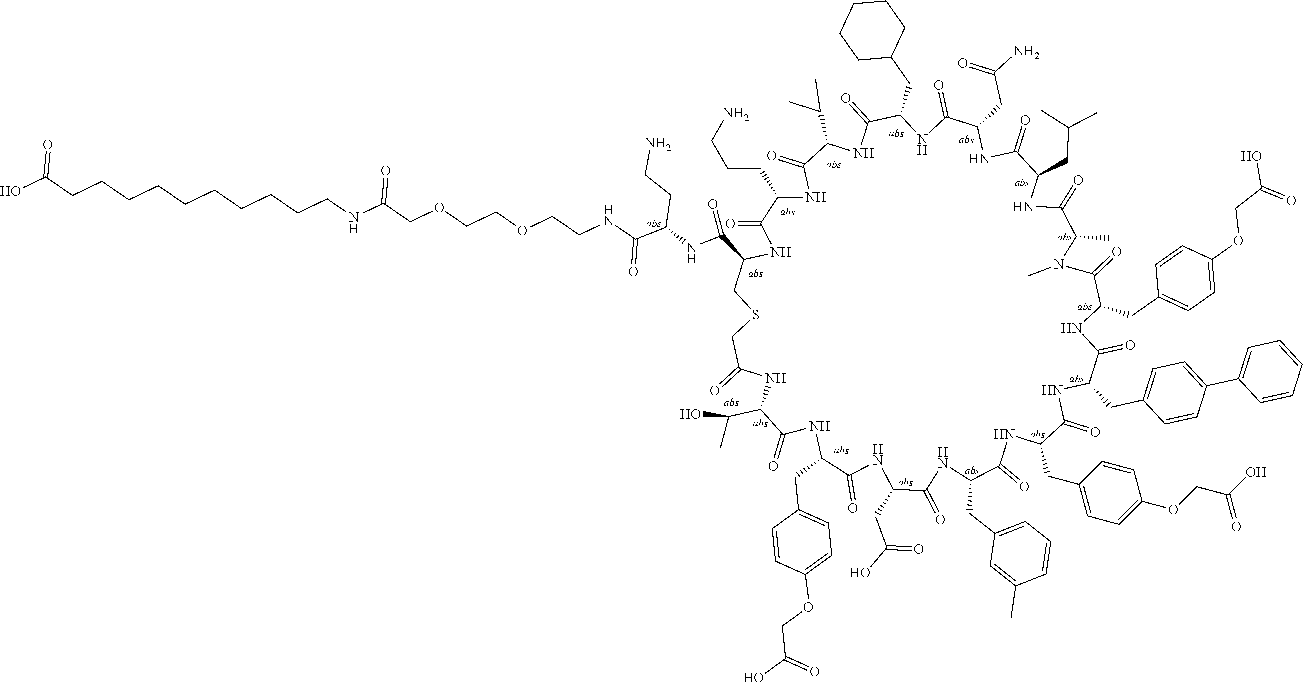 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1363 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 49.6 mg, and its estimated purity by LCMS analysis was 92.4%. Analysis condition B: Retention time=1.93 min; ESI-MS(+) m/z [M+2H]²⁺: 1267.1.

### Preparation of Example 1364

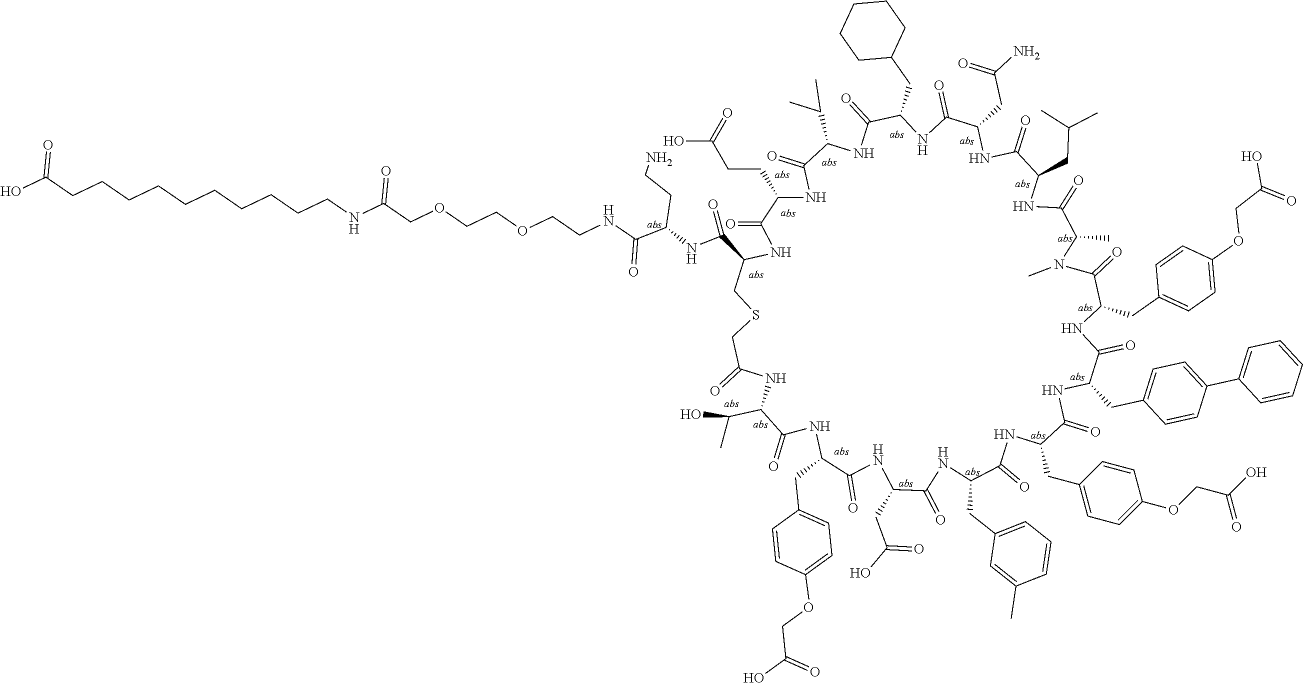 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1364 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 7% B, 7-47% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.9 mg, and its estimated purity by LCMS analysis was 94.9%. Analysis condition B: Retention time=2.06 min; ESI-MS(+) m/z [M+2H]²⁺: 1274.2.

### Preparation of Example 1365

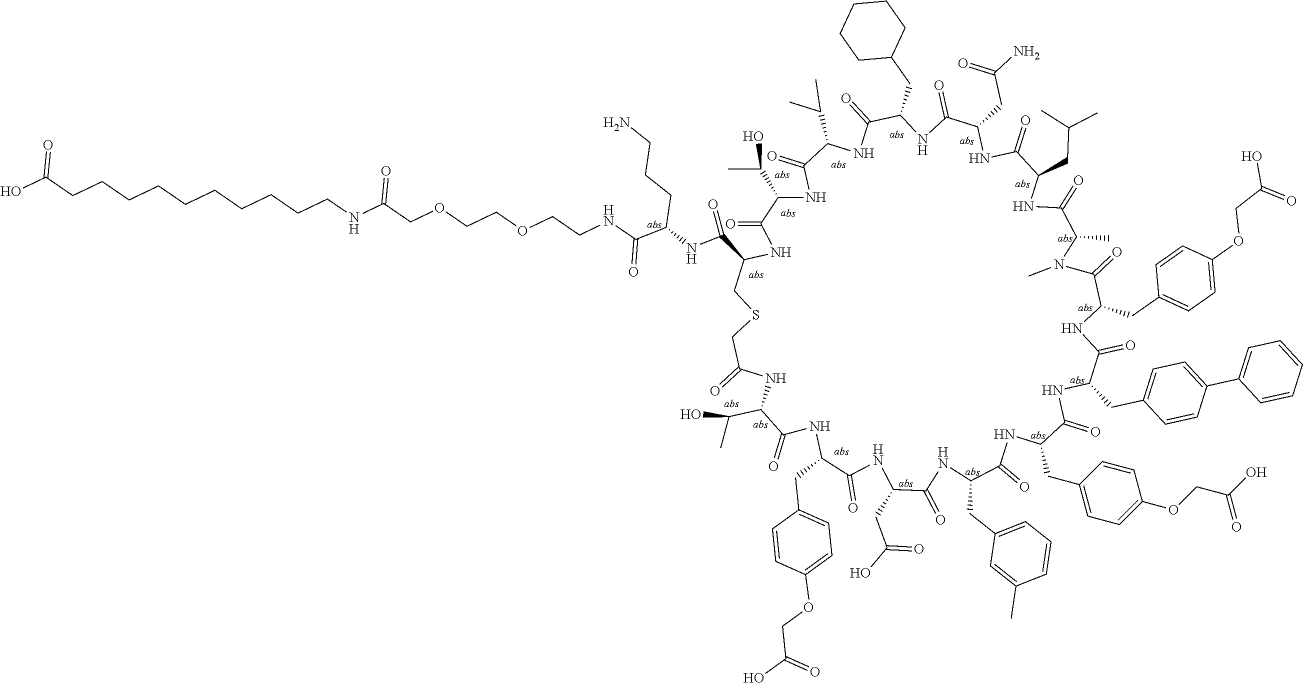 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1365 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 29.6 mg, and its estimated purity by LCMS analysis was 97.8%. Analysis condition B: Retention time=1.97 min; ESI-MS(+) m/z [M+2H]²⁺: 1267.2.

### Preparation of Example 1366

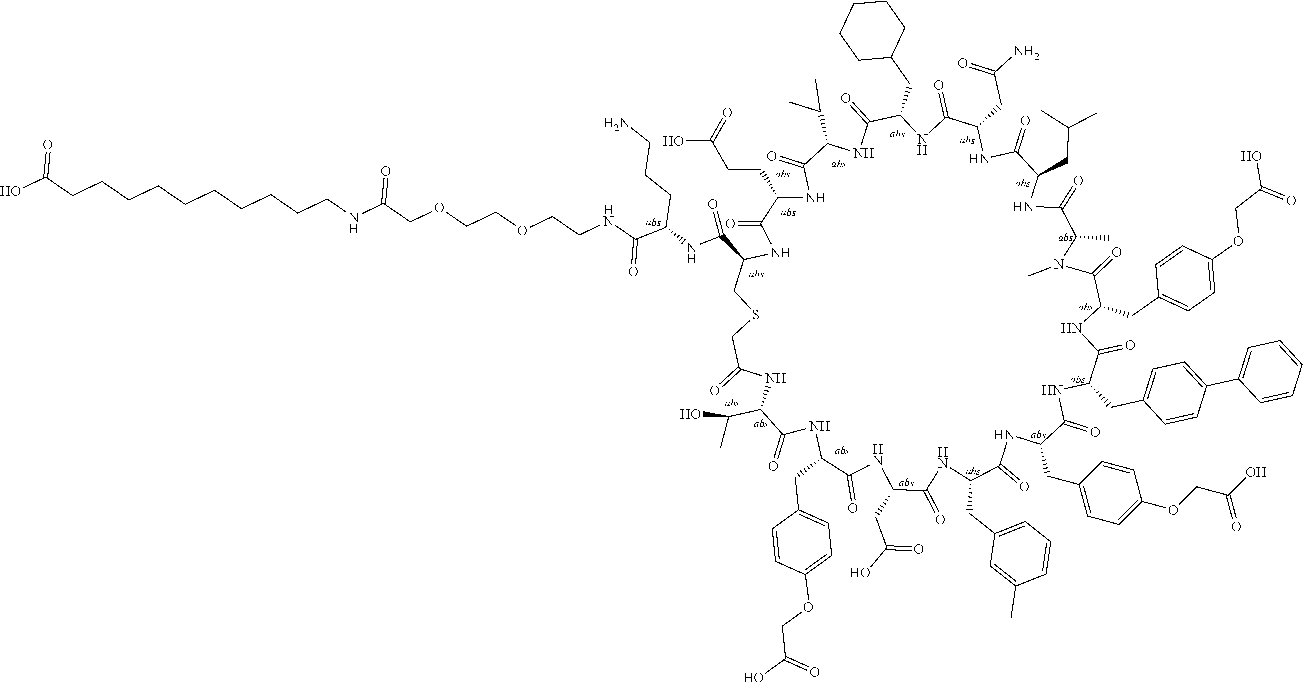 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1366 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 10% B, 10-50% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 62.9 mg, and its estimated purity by LCMS analysis was 97.4%. Analysis condition B: Retention time=1.96 min; ESI-MS(+) m/z [M+2H]²⁺: 1282.1.

### Preparation of Example 1367

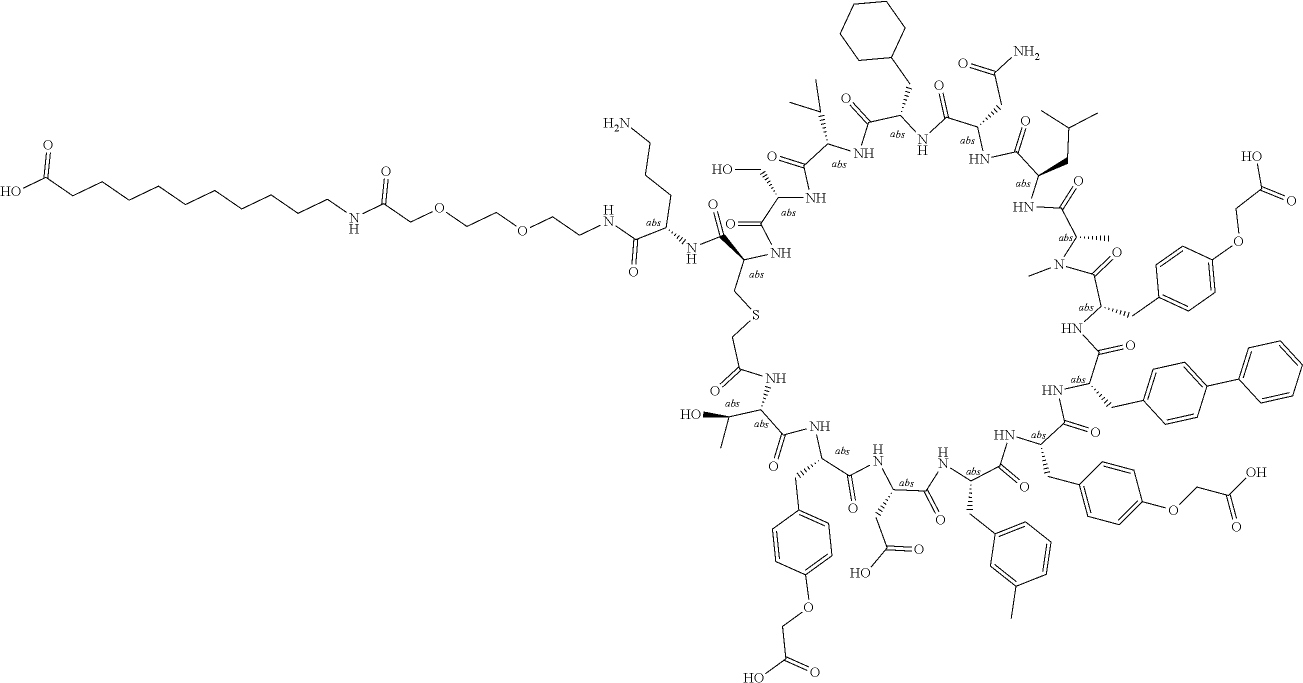 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC3CCCCC3)NC(=O)[C@H](CC(N)=O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1367 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.8 mg, and its estimated purity by LCMS analysis was 92%. Analysis condition A: Retention time=1.38 min; ESI-MS(+) m/z [M+2H]²⁺: 1260.4.

### Preparation of Example 1368

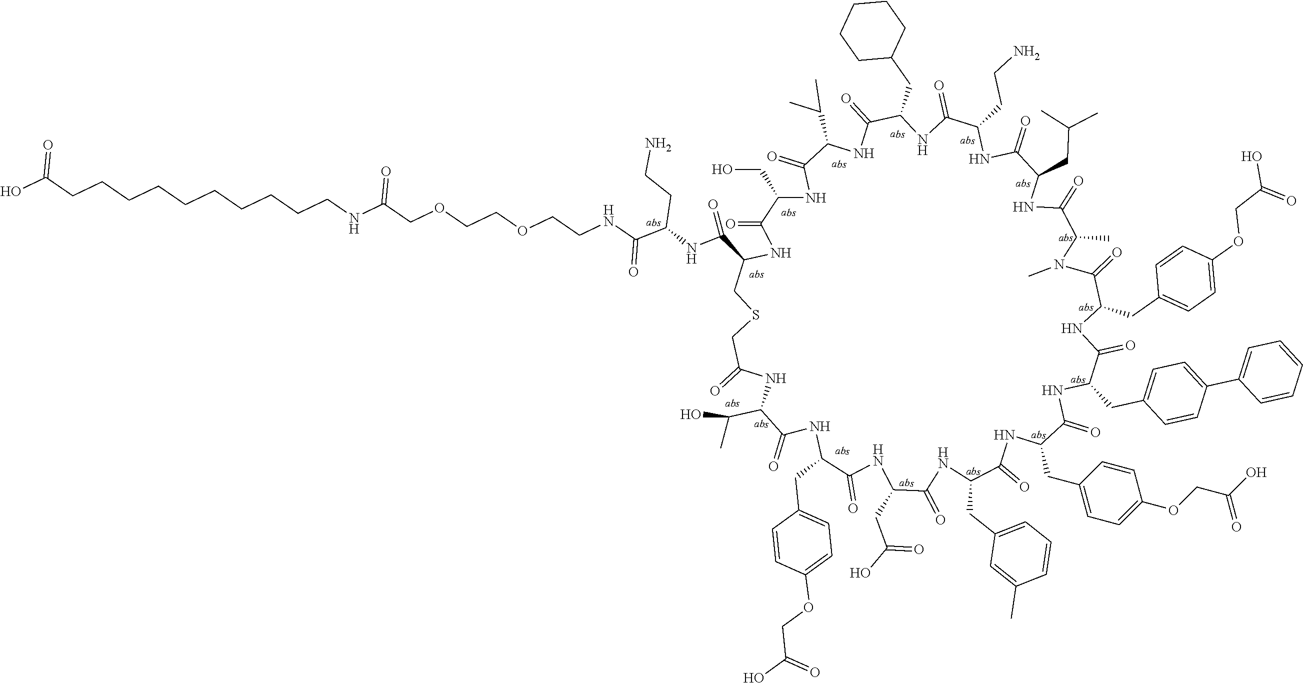 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1368 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 30% B, 30-70% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.4 mg, and its estimated purity by LCMS analysis was 94.7%. Analysis condition A: Retention time=1.4 min; ESI-MS(+) m/z [M+2H]²⁺: 1246.1.

### Preparation of Example 1369

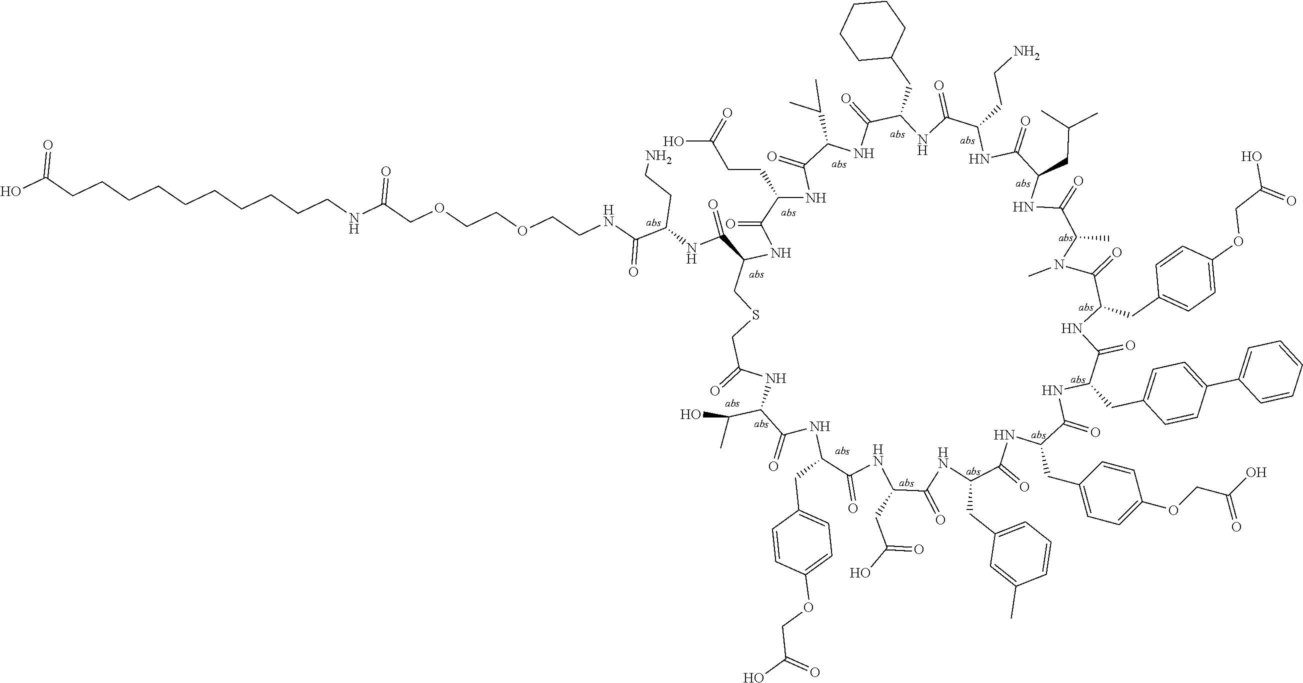 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1369 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 9% B, 9-49% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-pm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 26% B, 26-66% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.4 mg, and its estimated purity by LCMS analysis was 100%. Analysis condition A: Retention time=1.54 min; ESI-MS(+) m/z [M+2H]²⁺: 1267.1.

### Preparation of Example 1370

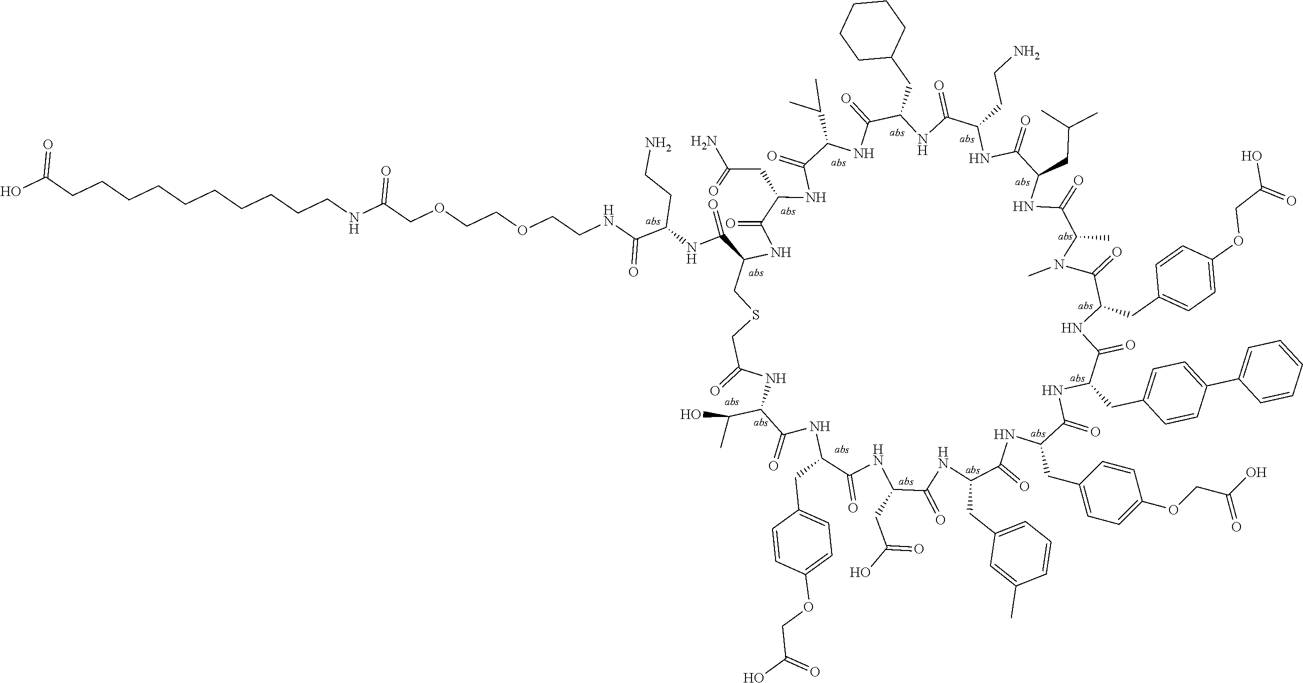 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1370 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 52.8 mg, and its estimated purity by LCMS analysis was 94.8%. Analysis condition B: Retention time=1.85 min; ESI-MS(+) m/z [M+2H]²⁺: 1260.

### Preparation of Example 1371

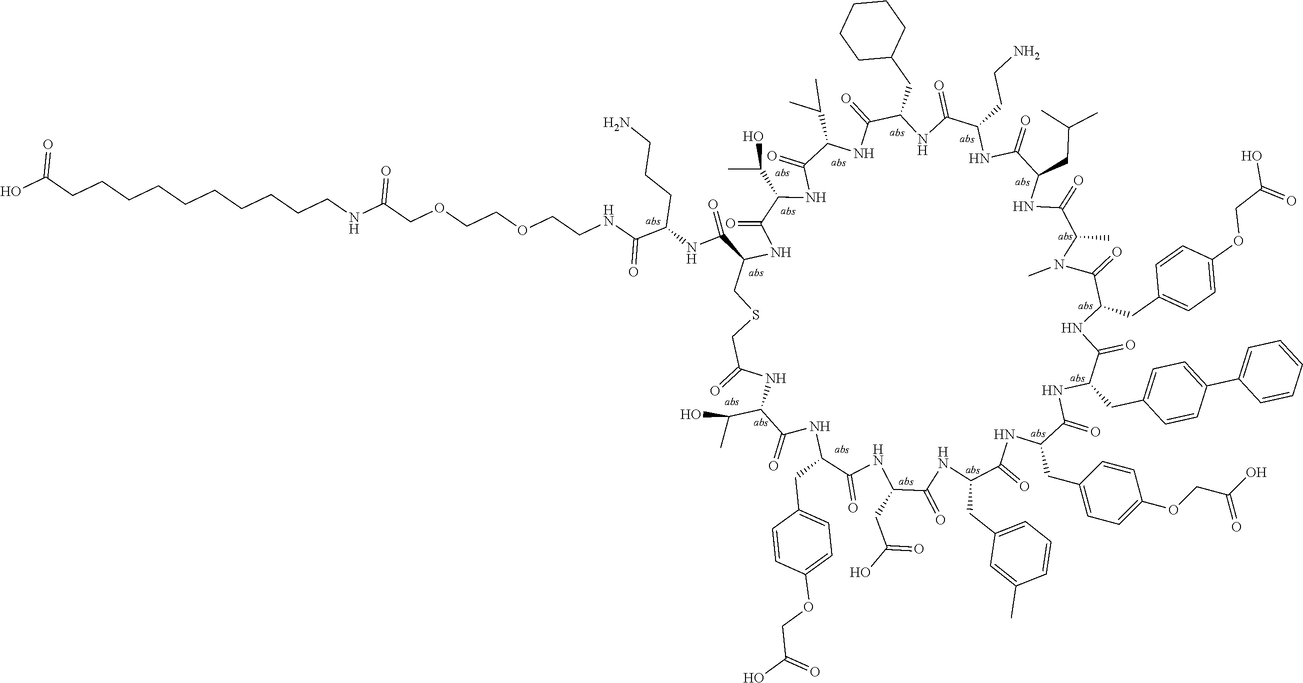 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1371 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 38.1 mg, and its estimated purity by LCMS analysis was 93.3%. Analysis condition B: Retention time=1.86 min; ESI-MS(+) m/z [M+2H]²⁺: 1260.2.

### Preparation of Example 1372

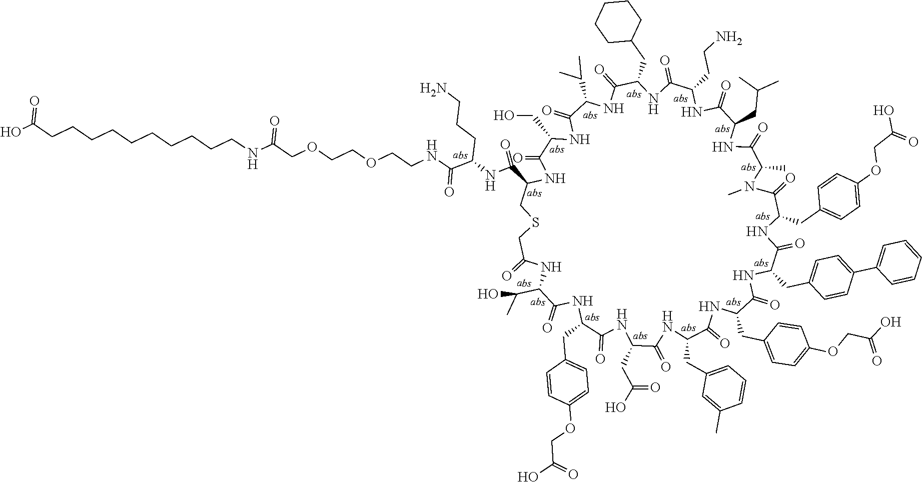 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1372 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 26% B, 26-66% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 14.9 mg, and its estimated purity by LCMS analysis was 98.5%. Analysis condition B: Retention time=1.82 min; ESI-MS(+) m/z [M+2H]²⁺: 1253.2.

### Preparation of Example 1373

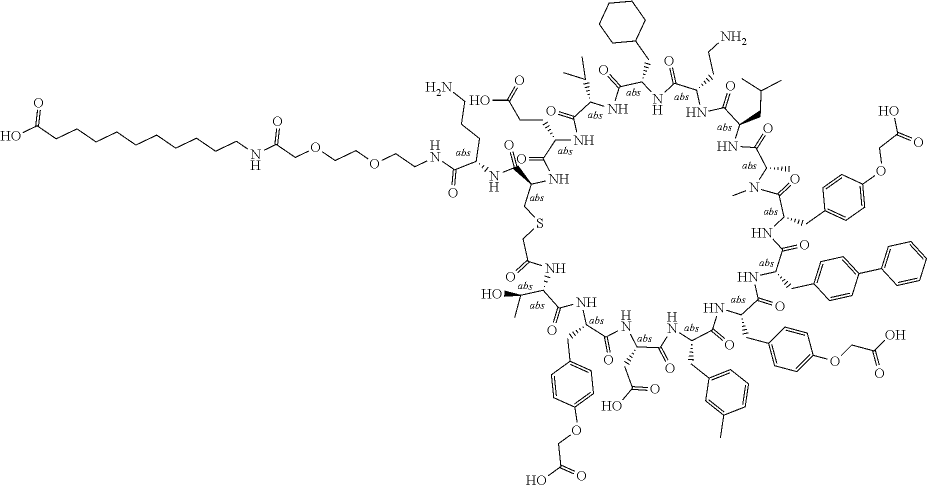 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1373 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20 mg, and its estimated purity by LCMS analysis was 100%. Analysis condition A: Retention time=1.41 min; ESI-MS(+) m/z [M+2H]²⁺: 1273.9.

### Preparation of Example 1374

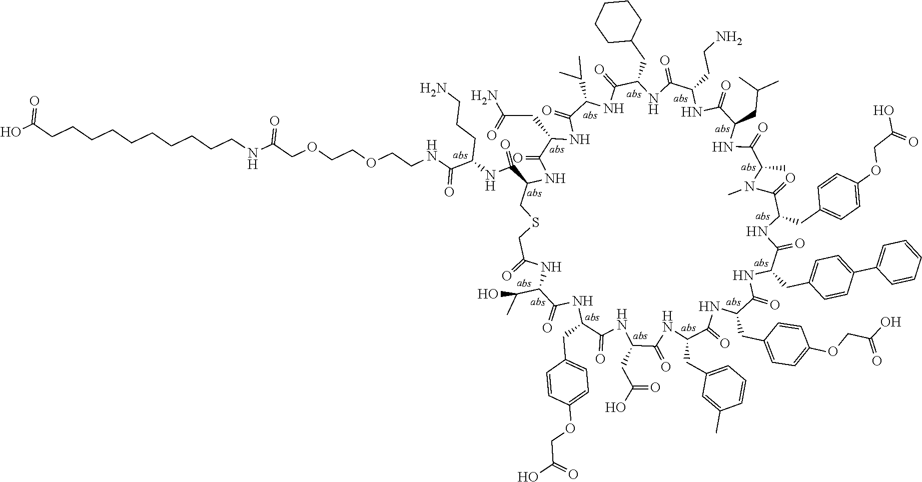 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1374 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 27% B, 27-67% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 44.4 mg, and its estimated purity by LCMS analysis was 97.10%. Analysis condition A: Retention time=1.58 min; ESI-MS(+) m/z [M+2H]²⁺: 1267.1.

### Preparation of Example 1375

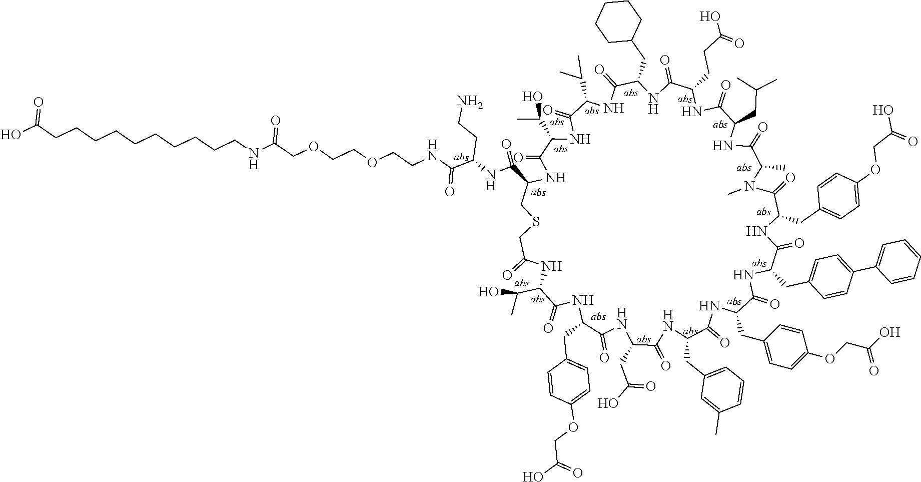 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1375 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 8% B, 8-48% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24 mg, and its estimated purity by LCMS analysis was 95.6%. Analysis condition B: Retention time=2.07 min; ESI-MS(+) m/z [M+2H]²⁺: 1268.1.

### Preparation of Example 1376

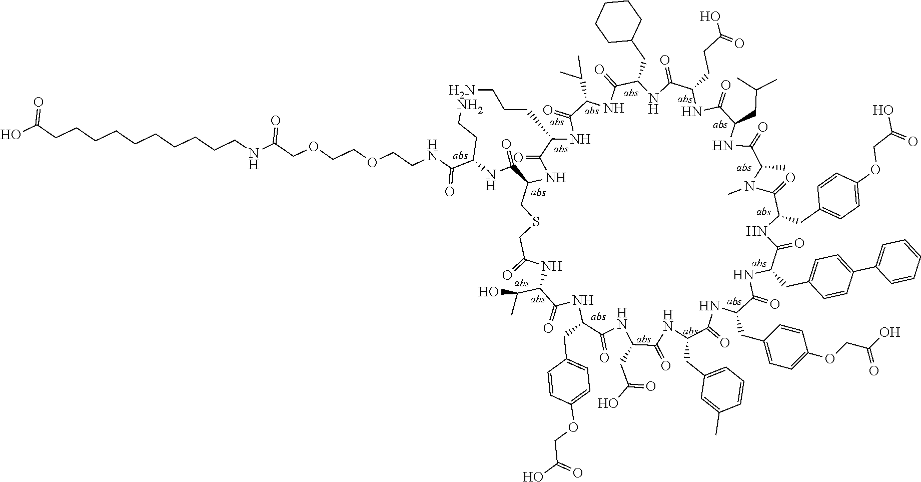 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCC(=O)O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1376 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.7 mg, and its estimated purity by LCMS analysis was 94.9%. Analysis condition A: Retention time=1.4 min; ESI-MS(+) m/z [M+2H]²⁺: 1274.2.

### Preparation of Example 1377

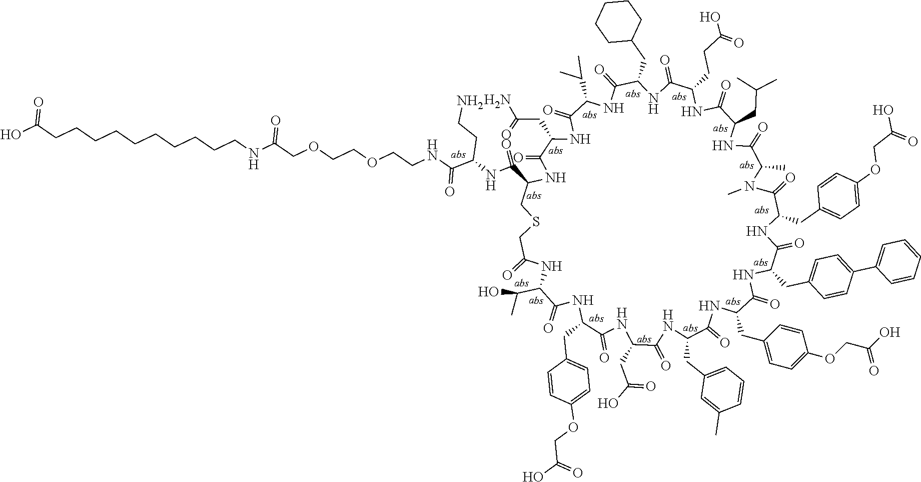 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CC(N)=O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1377 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 7% B, 7-47% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 23.2 mg, and its estimated purity by LCMS analysis was 98.7%. Analysis condition B: Retention time=1.95 min; ESI-MS(+) m/z [M+2H]²⁺: 1275.1.

### Preparation of Example 1378

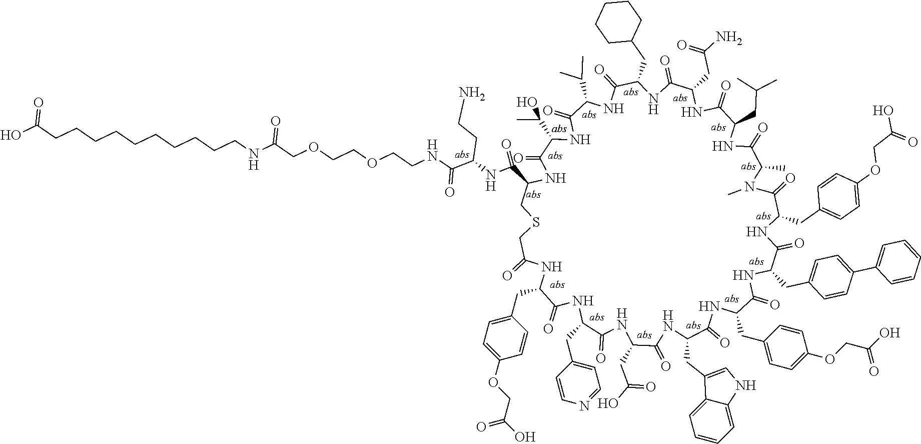 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CC(N)=O)NC1=O]

Example 1378 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 26% B, 26-66% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 44.8 mg, and its estimated purity by LCMS analysis was 92.2%. Analysis condition A: Retention time=1.39 min; ESI-MS(+) m/z [M+2H]²⁺: 1296.1.

### Preparation of Example 1379

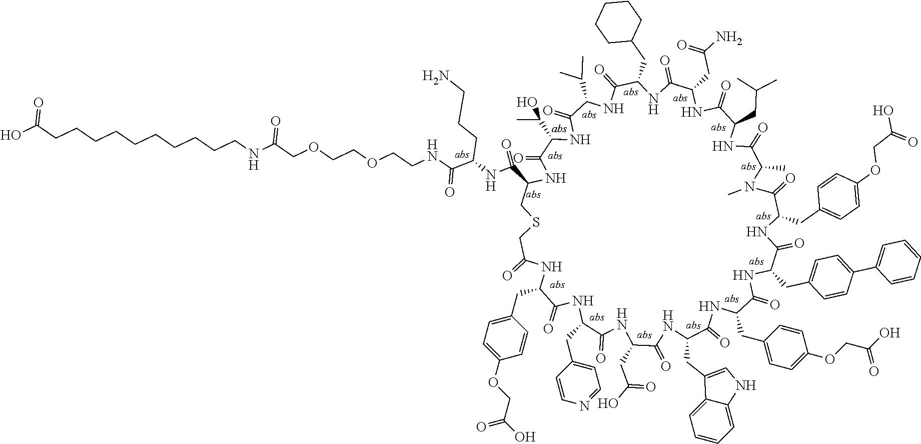 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CC(N)=O)NC1=O]

Example 1379 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.5 mg, and its estimated purity by LCMS analysis was 100%. Analysis condition A: Retention time=1.48 min; ESI-MS(+) m/z [M+2H]²⁺: 1303.1.

### Preparation of Example 1380

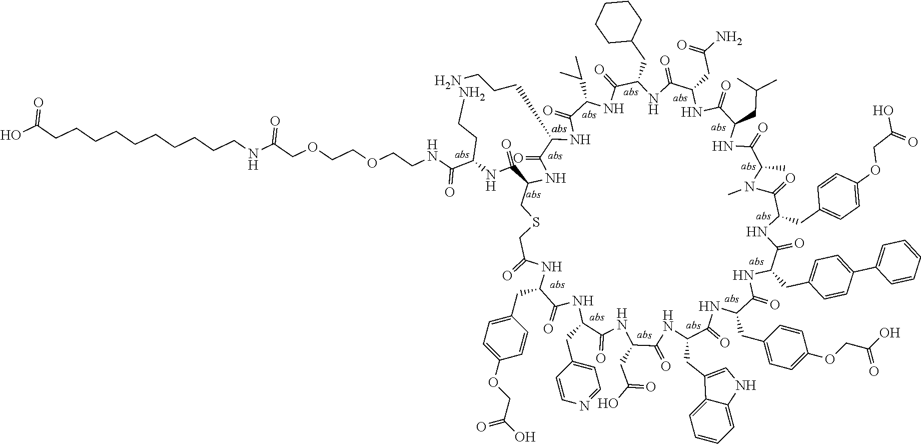 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Example 1380 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 22% B, 22-62% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 1400 B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10.4 mg, and its estimated purity by LCMS analysis was 100%. Analysis condition A: Retention time=1.4 min; ESI-MS(+) m/z [M+2H]²⁺: 1303.1.

### Preparation of Example 1381

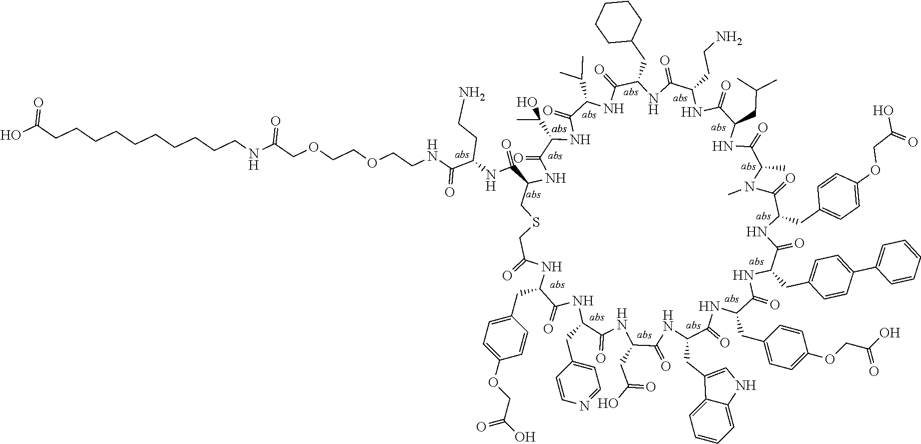 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1381 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 37 mg, and its estimated purity by LCMS analysis was 96.5%. Analysis condition B: Retention time=1.78 min; ESI-MS(+) m/z [M+2H]²⁺: 860.1.

### Preparation of Example 1382

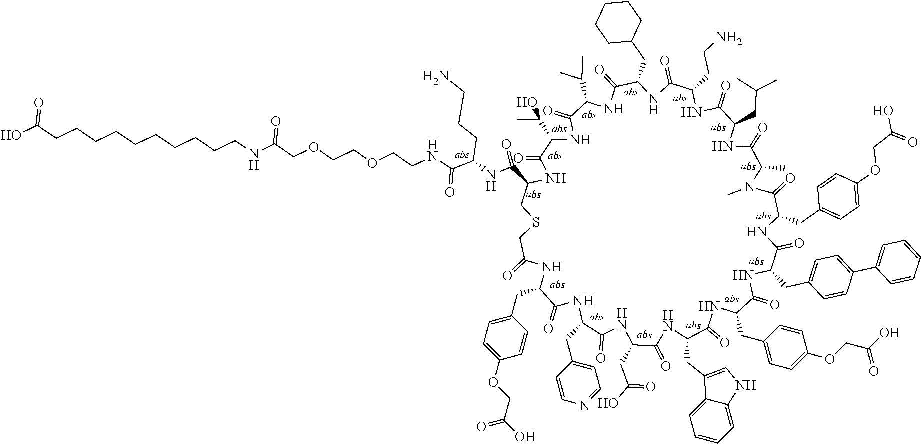 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1382 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 20% B, 20-60% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-pm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 14.5 mg, and its estimated purity by LCMS analysis was 95.3%. Analysis condition B: Retention time=1.73 min; ESI-MS(+) m/z [M+2H]²⁺: 1296.4.

### Preparation of Example 1383

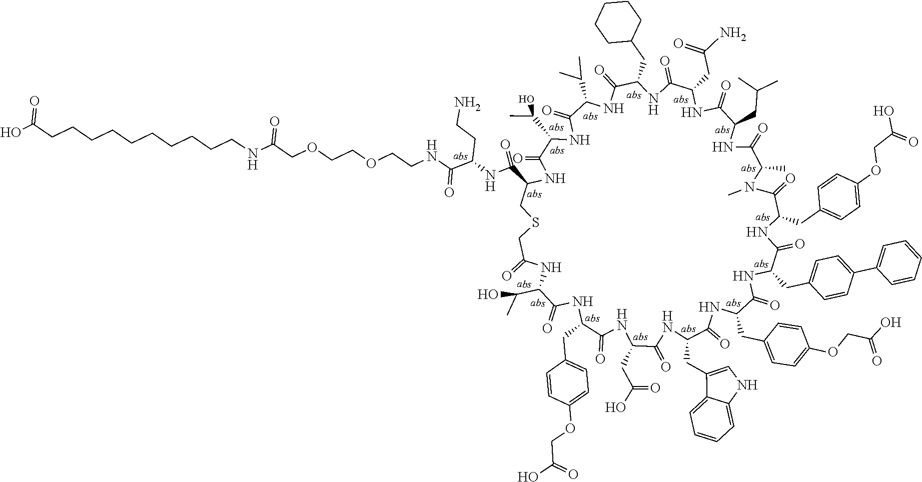 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CC(N)=O)NC1=O]

Example 1383 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 22 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33.2 mg, and its estimated purity by LCMS analysis was 94.9%. Analysis condition A: Retention time=1.5 min; ESI-MS(+) m/z [M+2H]²⁺: 1272.9.

### Preparation of Example 1384

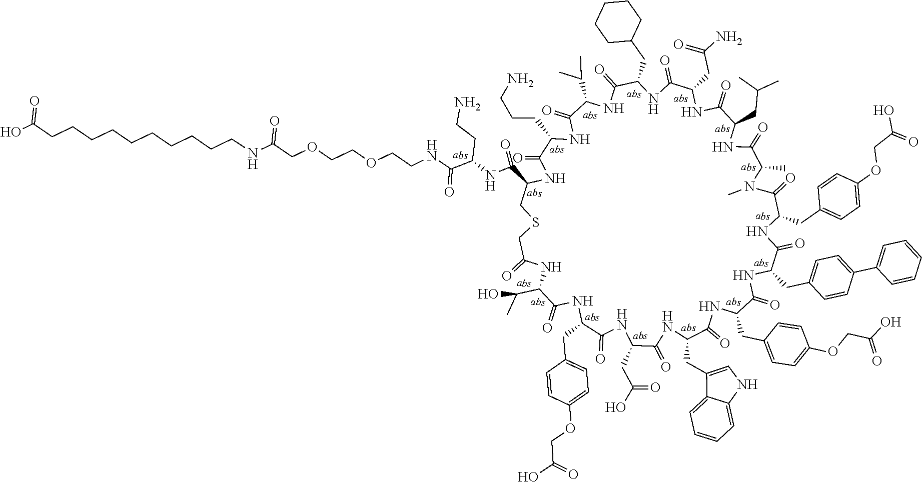 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Example 1384was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 26% B, 26-66% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.1 mg, and its estimated purity by LCMS analysis was 95.5%. Analysis condition B: Retention time=1.89 min; ESI-MS(+) m/z [M+2H]²⁺: 1280.3.

### Preparation of Example 1385

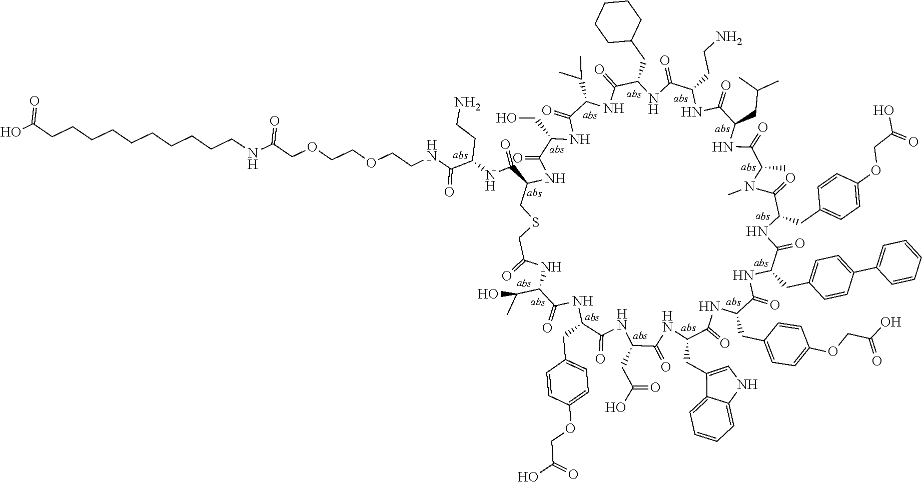 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 1385 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 29% B, 29-69% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 11.6 mg, and its estimated purity by LCMS analysis was 0%. Analysis condition B: Retention time=1.94 min; ESI-MS(+) m/z [M+2H]²⁺: 1266.1.

### Preparation of Example 1386

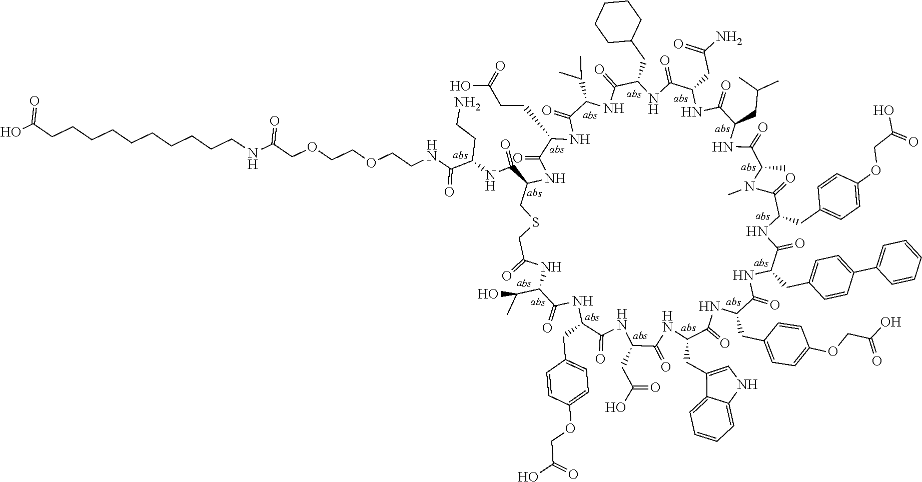 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CC(N)=O)NC1=O]

Example 1386 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 9% B, 9-49% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 24.1 mg, and its estimated purity by LCMS analysis was 97.6%. Analysis condition B: Retention time=1.97 min; ESI-MS(+) m/z [M+2H]²⁺: 1286.9.

### Preparation of Example 1387

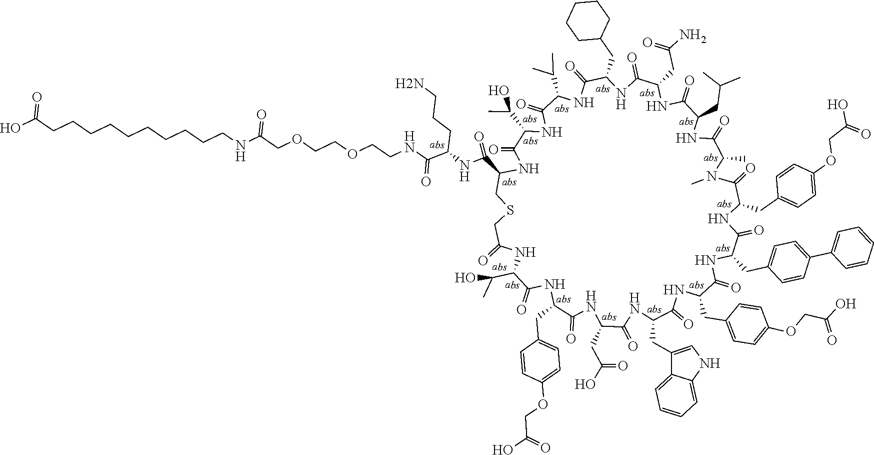 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CC(N)=O)NC1=O]

Example 1387 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 29% B, 29-69% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 47.2 mg, and its estimated purity by LCMS analysis was 95.3%. Analysis condition A: Retention time=1.42 min; ESI-MS(+) m/z [M+2H]²⁺: 1280.1.

### Preparation of Example 1388

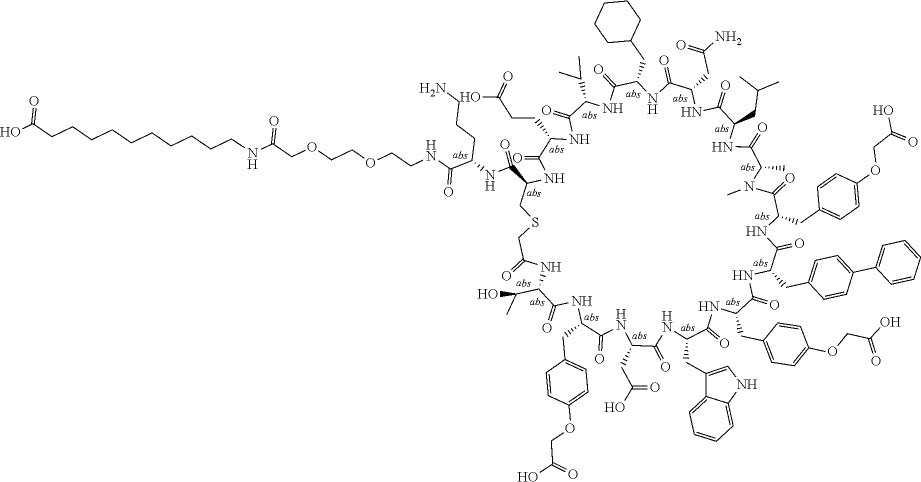 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CC(N)=O)NC1=O]

Example 1388 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 9% B, 9-49% B over 22 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 35.9 mg, and its estimated purity by LCMS analysis was 99.10%. Analysis condition B: Retention time=1.96 min; ESI-MS(+) m/z [M+2H]²⁺: 1294.2.

### Preparation of Example 1389

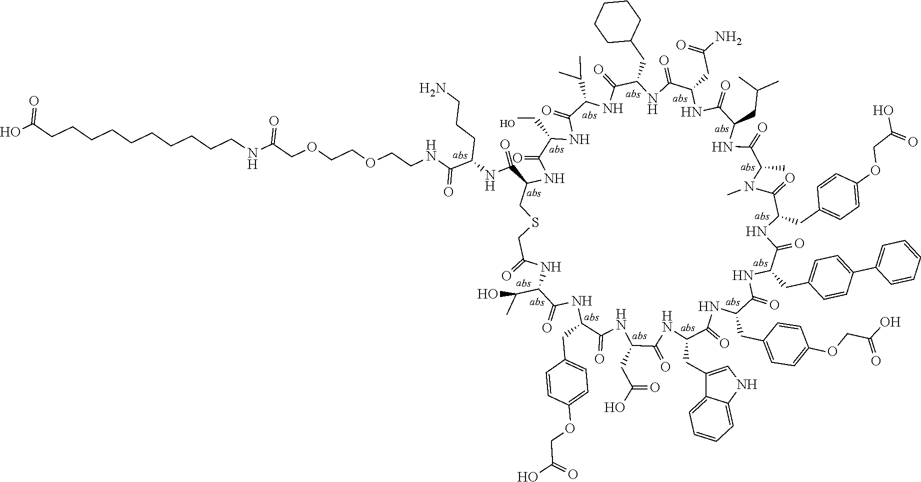 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC1=O]

Example 1389 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.2 mg, and its estimated purity by LCMS analysis was 94.60%. Analysis condition A: Retention time=1.37 min; ESI-MS(+) m/z [M+2H]²⁺: 1273.2.

### Preparation of Example 1390

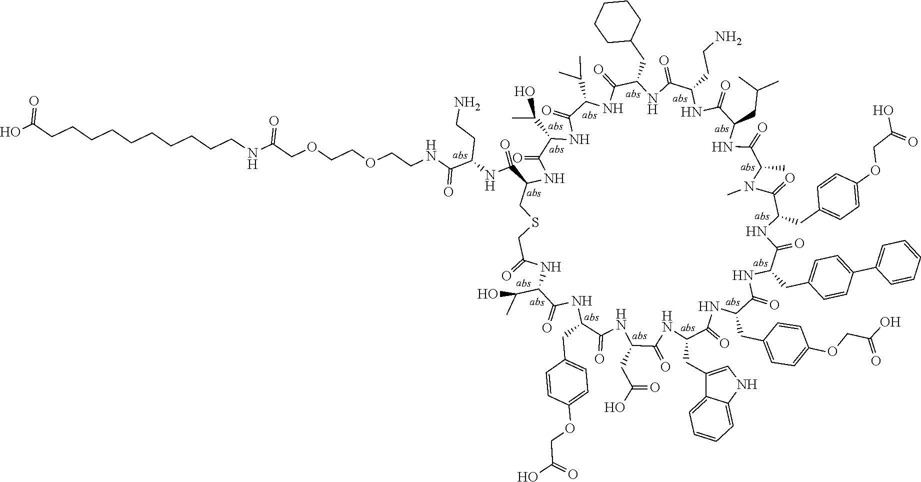 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1390 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 23% B, 23-63% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 88 mg, and its estimated purity by LCMS analysis was 93.6%. Analysis condition A: Retention time=1.6 min; ESI-MS(+) m/z [M+2H]²⁺: 1266.2.

### Preparation of Example 1391

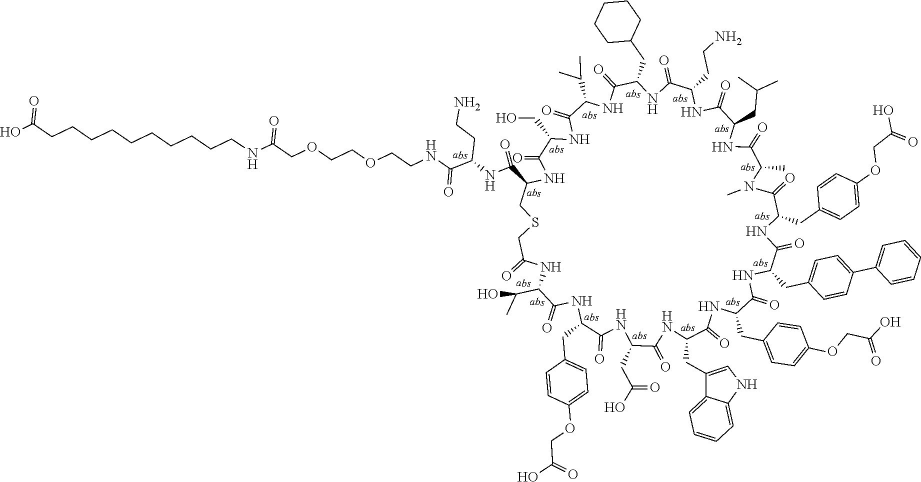 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 1391 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile: water with 0.1% trifluoroacetic acid; Gradient: a 0-minute hold at 21% B, 21-51% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.7 mg, and its estimated purity by LCMS analysis was 98.1%. Analysis condition B: Retention time=1.89 min; ESI-MS(+) m/z [M+2H]²⁺: 1259.

### Preparation of Example 1392

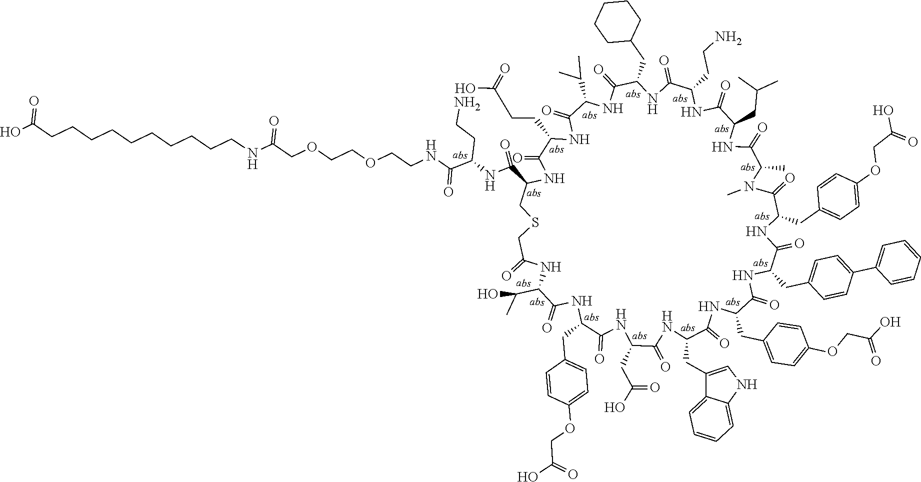 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1392 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 48.1 mg, and its estimated purity by LCMS analysis was 90.3%. Analysis condition B: Retention time=1.81 min; ESI-MS(+) m/z [M+2H]²⁺: 1280.

### Preparation of Example 1393

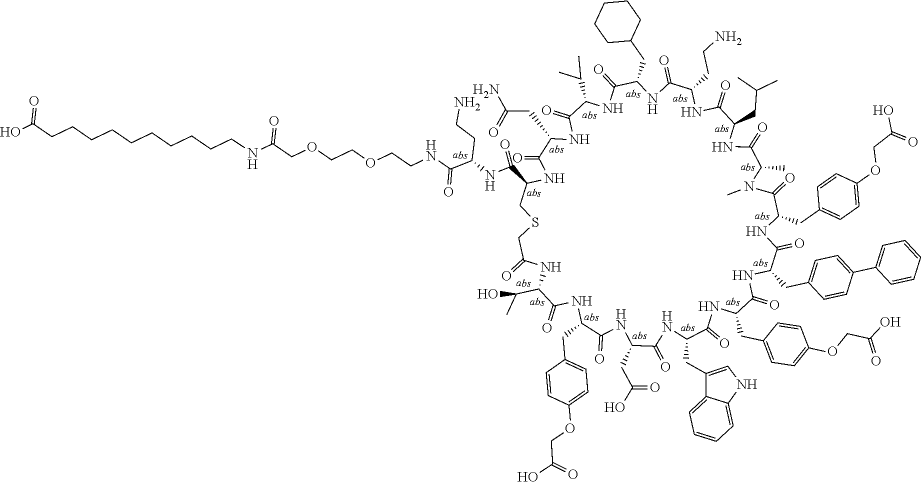 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CC(N)=O)NC(=O)[C@H](C(C)C)NC(=O)[C@H](CC2CCCCC2)NC(=O)[C@H](CCN)NC1=O]

Example 1393 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 34.4 mg, and its estimated purity by LCMS analysis was 95%. Analysis condition B: Retention time=1.88 min; ESI-MS(+) m/z [M+2H]²⁺: 1272.2.

### Preparation of Example 1394

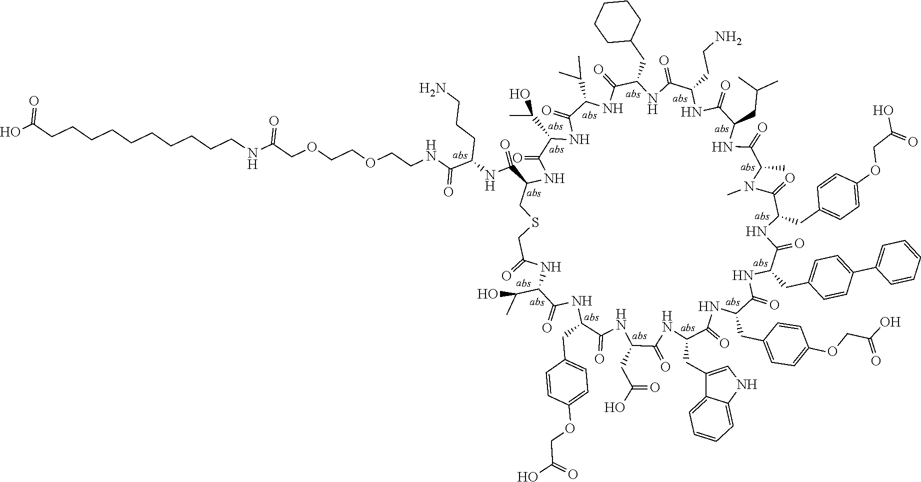 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Example 1394 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 24% B, 24-64% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 36.6 mg, and its estimated purity by LCMS analysis was 100%. Analysis condition A: Retention time=1.6 min; ESI-MS(+) m/z [M+2H]²⁺: 1273.2.

### Preparation of Example 1395

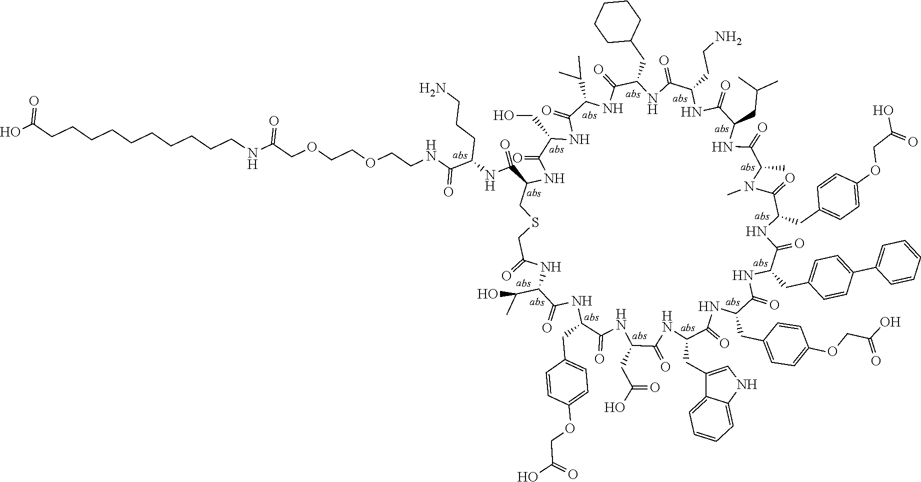 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 1395 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 30 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 26% B, 26-66% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 22.5 mg, and its estimated purity by LCMS analysis was 94.8%. Analysis condition B: Retention time=1.82 min; ESI-MS(+) m/z [M+2H]²⁺: 1266.1.

### Preparation of Example 1396

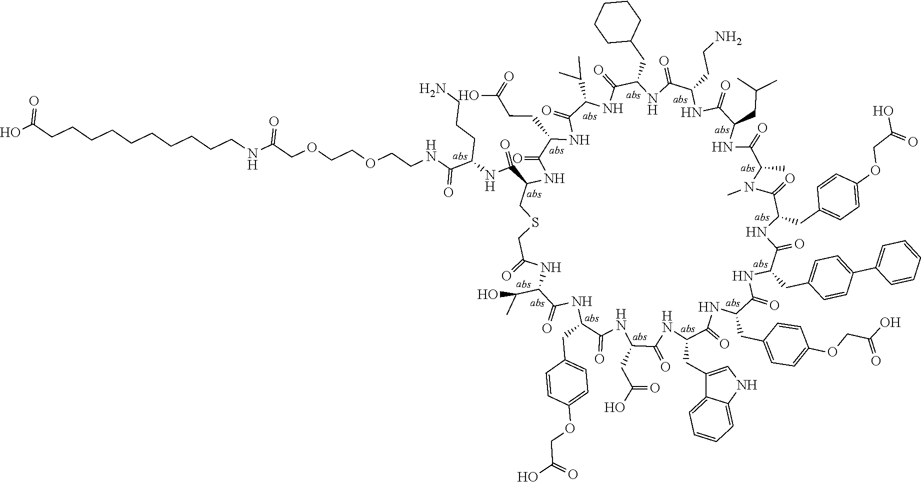 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 1396 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 10%0 B, 10-50% B over 22 minutes, then a 0-minute hold at 100% a B; Flow Rate: 20 mL/min; Column Temperature: 25 C. Fraction collection was triggered by MS signals. Fractions containing the desired product were combined and dried via centrifugal evaporation. The yield of the product was 39.8 mg, and its estimated purity by LCMS analysis was 95.8% a. Analysis condition B: Retention time=1.83 min; ESI-MS(+) m/z [M+2H]²⁺: 1287.1.

### Preparation of Example 1397

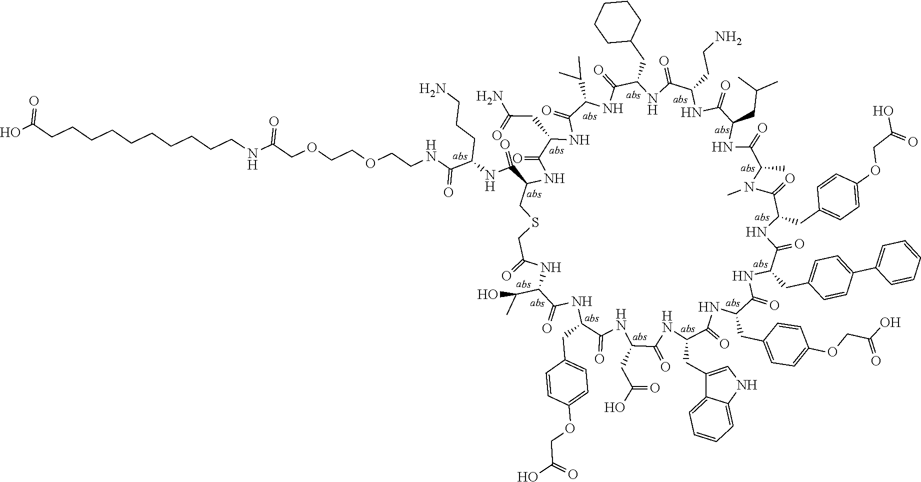 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCCN)C(=O)NCCOCCOCC(=O)NCCCCCCCCCCC(=O)O)NC(=O)[C@H](CC(N)=O)NC(=O)[C@H](C(C)C)NC(=O)[C@H](CC2CCCCC2)NC(=O)[C@H](CCN)NC1=O]

Example 1397 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 24% B, 24-64% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 150% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 22.2 mg, and its estimated purity by LCMS analysis was 98.8%. Analysis condition B: Retention time=1.8 min; ESI-MS(+) m/z [M+2H]²⁺: 1279.9.

### Preparation of Example 1398

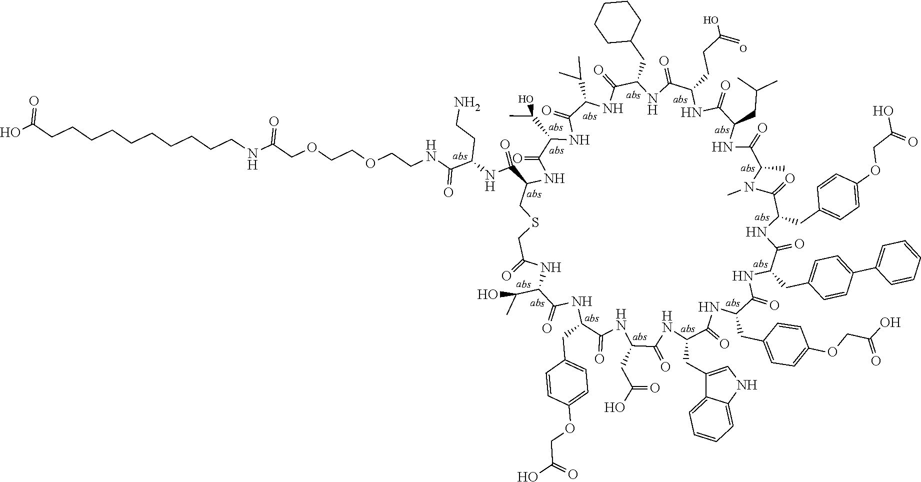 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCC(=O)O)NC1=O]

Example 1398 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 29% B, 29-69% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-pm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-43% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23 mg, and its estimated purity by LCMS analysis was 100%. Analysis condition A: Retention time=1.53 min; ESI-MS(+) m/z [M+2H]²⁺: 1281.1.

### Preparation of Example 1399

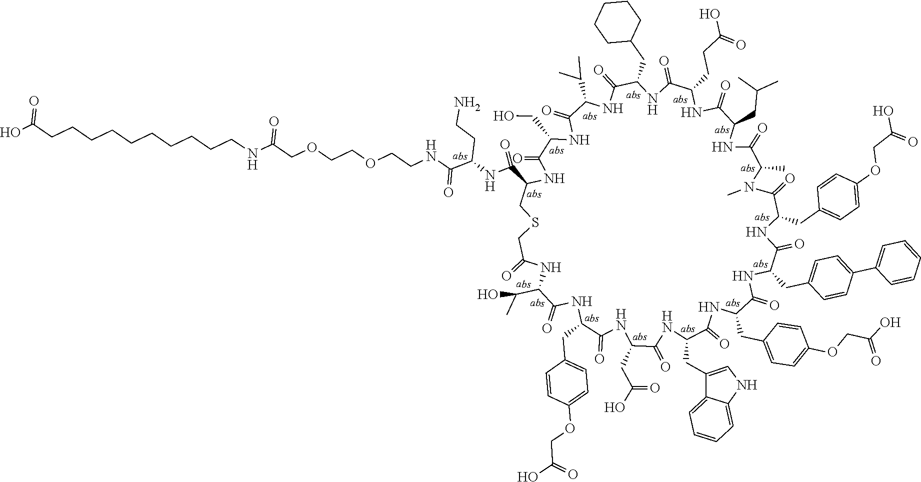 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCC(=O)O)NC1=O]

Example 1399 was prepared, using 2-Chlorotrityl resin pre-loaded with 11-((((9H-fluoren-9-yl)methoxy)carbonyl)amino)undecanoic acid on a 50 gmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 27% B, 27-67% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by UV signals. Fractions containing the desired product were combined and dried via centrifugal evaporation. The yield of the product was 7.2 mg, and its estimated purity by LCMS analysis was 95.2%. Analysis condition B: Retention time=1.96 min; ESI-MS(+) m/z [M+2H]²⁺: 1274.2.

### Preparation of Example 1400

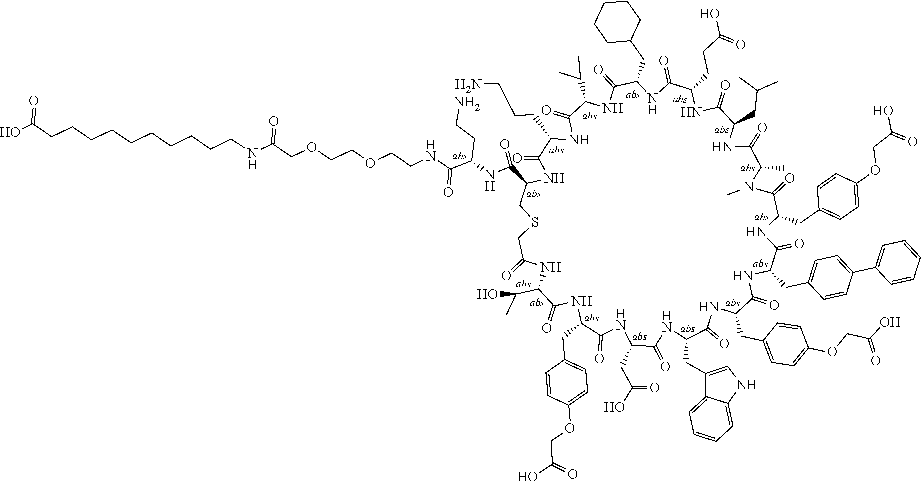 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCC(=O)O)NC1=O]

Example 1400 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 25% B, 25-65% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by UV signals. Fractions containing the desired product were combined and dried via centrifugal evaporation. The yield of the product was 16.8 mg, and its estimated purity by LCMS analysis was 91.2%. Analysis condition A: Retention time=1.42 min; ESI-MS(+) m/z [M+2H]²⁺: 1287.1.

### Preparation of Example 1401

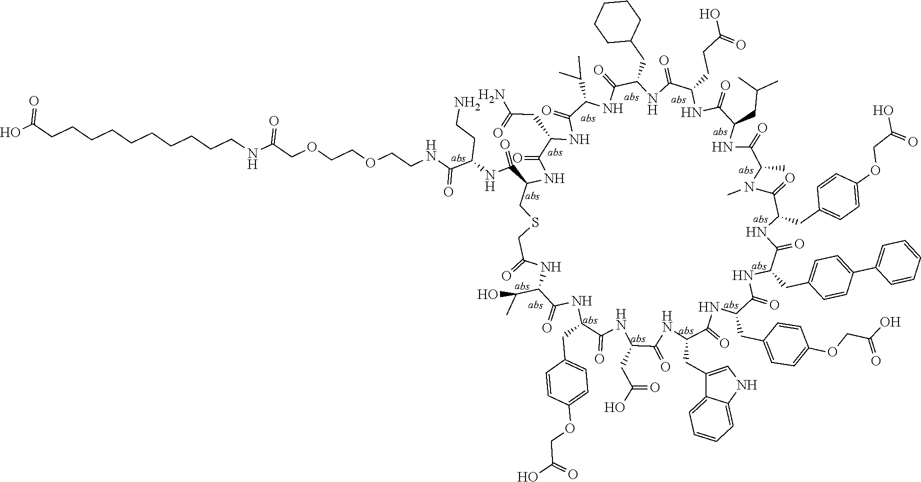 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CC(N)=O)NC(=O)[C@H](C(C)C)NC(=O)[C@H](CC2CCCCC2)NC(=O)[C@H](CCC(=O)O)NC1=O]

Example 1401 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 9% B, 9-49% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.4 mg, and its estimated purity by LCMS analysis was 99%. Analysis condition A: Retention time=1.5 min; ESI-MS(+) m/z [M+2H]²⁺: 1287.1.

### Preparation of Example 1402

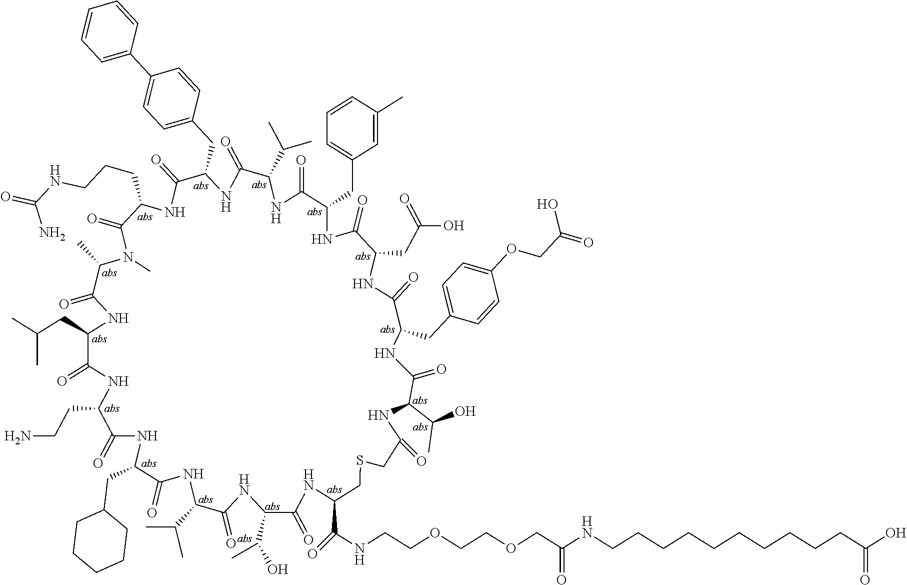 [SMILES: Cc1cccc(C[C@@H]2NC(=O)[C@H](CC(=O)O)NC(=O)[C@H](Cc3ccc(OCC(=O)O)cc3)NC(=O)[C@@H]([C@H](C)O)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 1402 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 23% B, 23-63% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 47.8 mg, and its estimated purity by LCMS analysis was 98.3%. Analysis condition B: Retention time=1.96 min; ESI-MS(+) m/z [M+2H]²⁺: 1110.1.

### Preparation of Example 1403

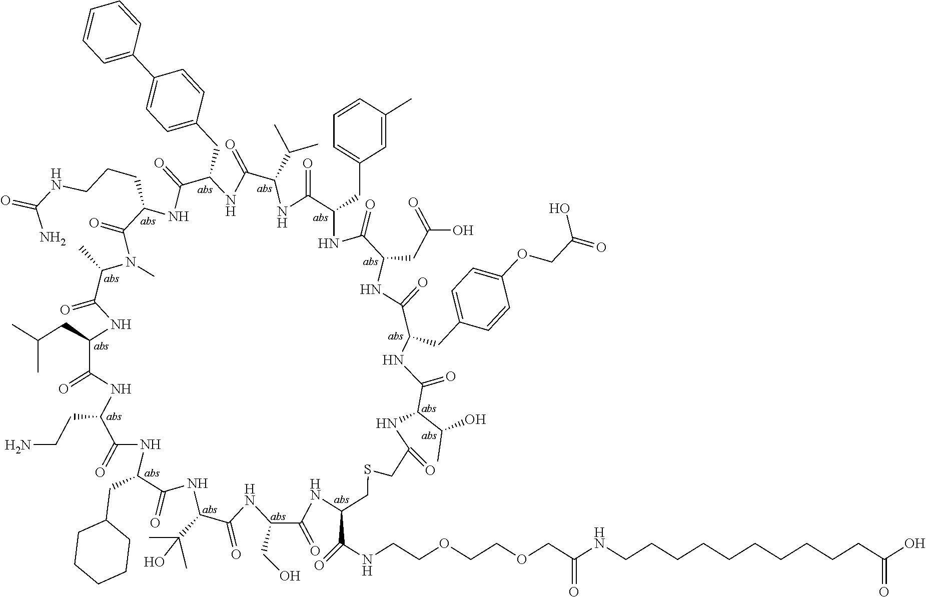 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 1403 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 22% B, 22-62% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 30.6 mg, and its estimated purity by LCMS analysis was 96.5%. Analysis condition A: Retention time=1.71 min; ESI-MS(+) m/z [M+2H]²⁺: 1111.1.

### Preparation of Example 1404

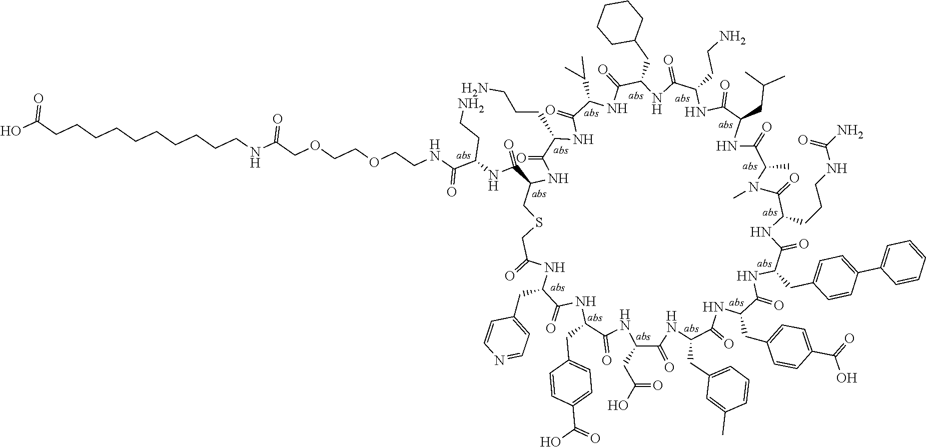 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(C(=O)O)cc3)NC2=O)c1]

Example 1404 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-50% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 20% B, 20-60% B over 20 minutes, then a 2-minute hold at 100% B; Flow Rate: 45 mL/min; Column Temperature: 25 C. Fraction collection was triggered by MS signals. Fractions containing the desired product were combined and dried via centrifugal evaporation. The yield of the product was 9.5 mg, and its estimated purity by LCMS analysis was 95.5%. Analysis condition B: Retention time=1.7 min; ESI-MS(+) m/z [M+2H]²⁺: 815.1.

### Preparation of Example 1405

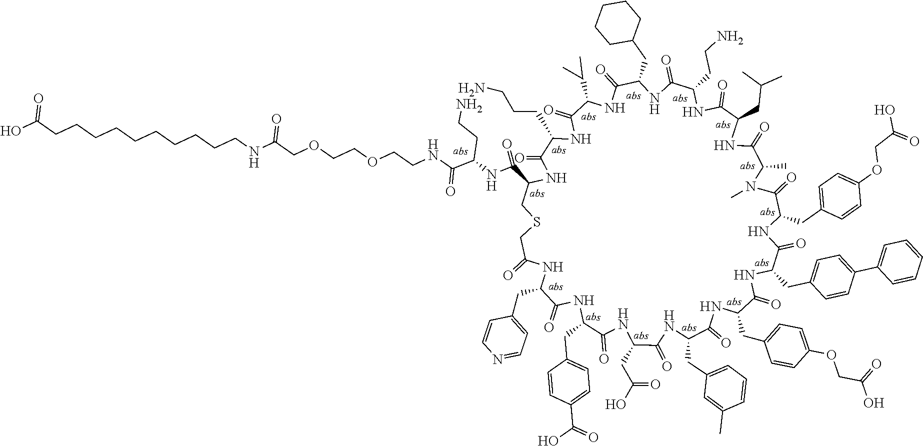 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1405 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 17% B, 17-55% B over 30 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: Waters CSH Fluoro Phenyl, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 2-minute hold at 100% B; Flow Rate: 45 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 95.7%. Analysis condition B: Retention time=1.71 min; ESI-MS(+) m/z [M+2H]²⁺: 1268.4.

### Preparation of Example 1406

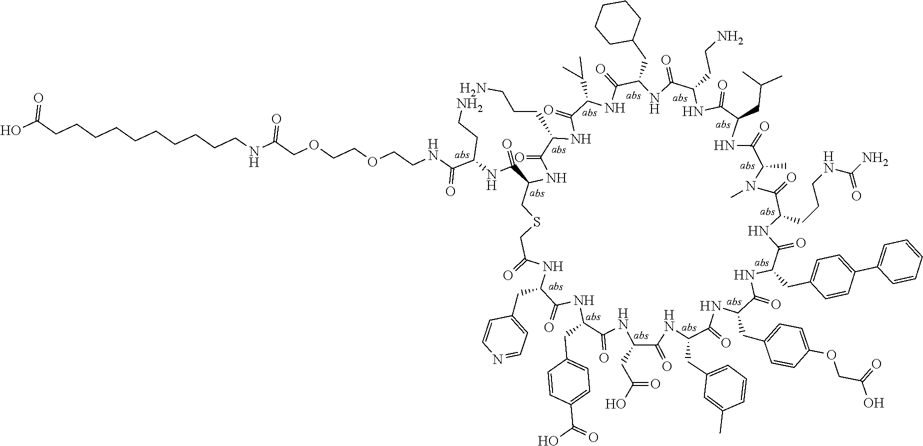 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(OCC(=O)O)cc3)NC2=O)c1]

Example 1406 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8.1 mg, and its estimated purity by LCMS analysis was 92.9%. Analysis condition A: Retention time=1.6 min; ESI-MS(+) m/z [M+2H]²⁺: 1236.4.

### Preparation of Example 1407

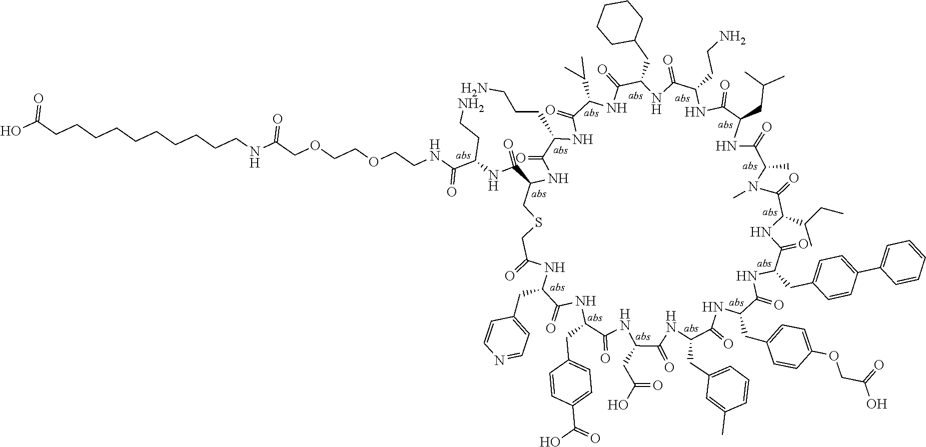 [SMILES: CCC(C)[C@@H]1NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 1407 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 21% B, 21-61% B over 20 minutes, then a 2-minute hold at 100% B; Flow Rate: 45 mL/min; Column Temperature: 25 C. Fraction collection was triggered by MS signals. Fractions containing the desired product were combined and dried via centrifugal evaporation. The yield of the product was 3.5 mg, and its estimated purity by LCMS analysis was 96%. Analysis condition A: Retention time=1.7 min; ESI-MS(+) m/z [M+3H]³⁺: 810.2.

### Preparation of Example 1408

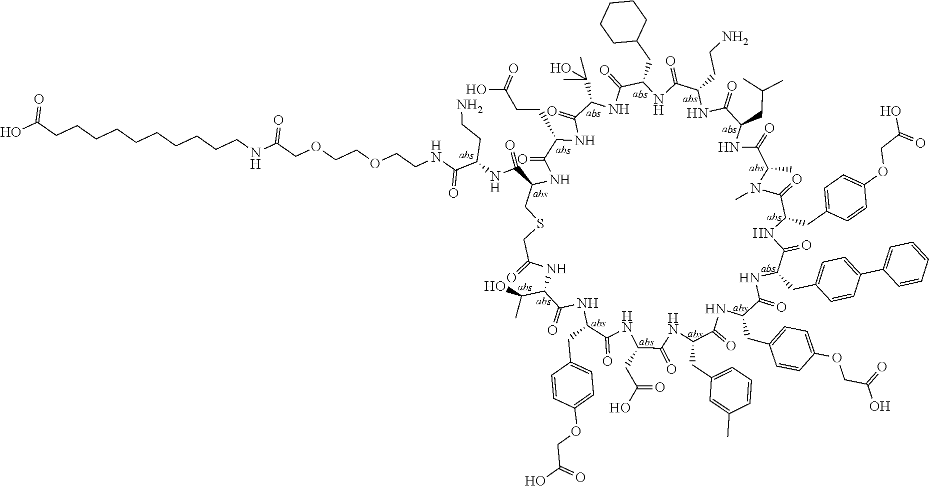 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1408 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 23.5 mg, and its estimated purity by LCMS analysis was 100%. Analysis condition A: Retention time=1.37 min; ESI-MS(+) m/z [M+2H]²⁺: 1276.1.

### Preparation of Example 1409

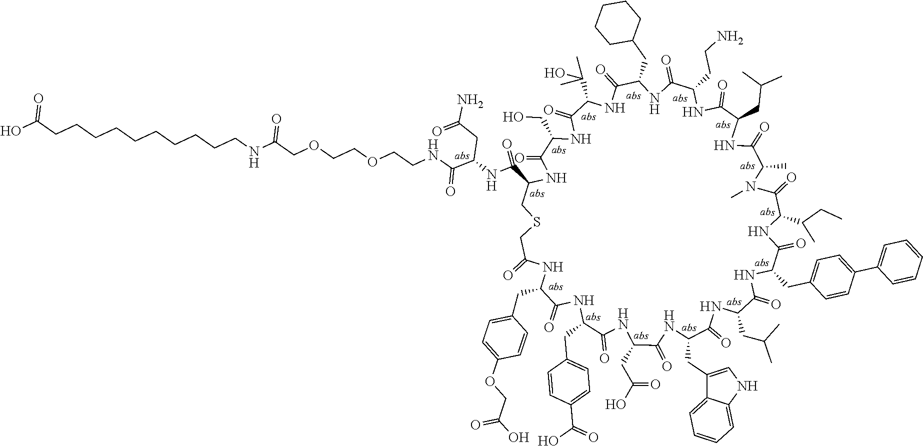 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 1409 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 33% B, 33-73% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 24% B, 24-64% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 21.1 mg, and its estimated purity by LCMS analysis was 93%. Analysis condition A: Retention time=1.76 min; ESI-MS(+) m/z [M+2H]²⁺: 1211.1.

### Preparation of Example 1410

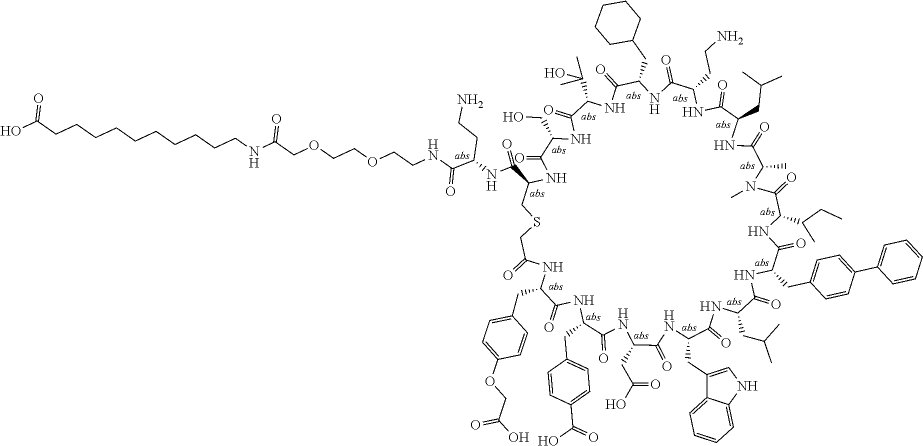 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 1410 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 24% B, 24-64% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.2 mg, and its estimated purity by LCMS analysis was 97.2%. Analysis condition B: Retention time=2.06 min; ESI-MS(+) m/z [M+2H]²⁺: 1204.2.

### Preparation of Example 1411

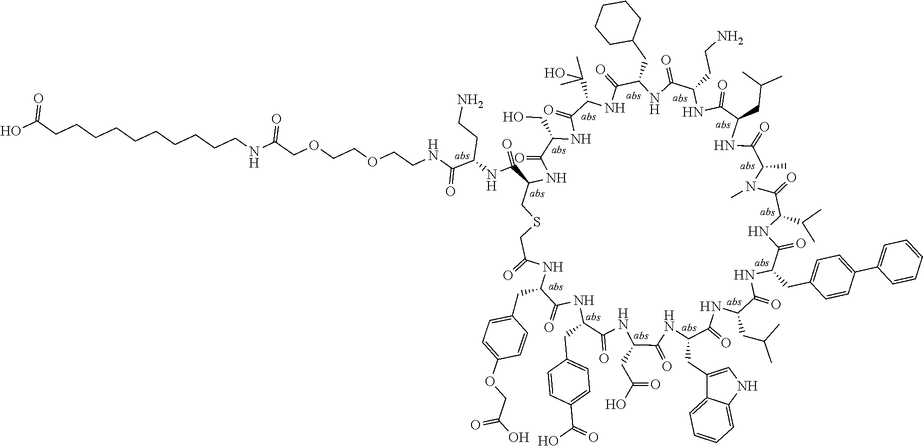 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1411 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 23% B, 23-63% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.7 mg, and its estimated purity by LCMS analysis was 95%. Analysis condition A: Retention time=1.77 min; ESI-MS(+) m/z [M+2H]²⁺: 1197.3.

### Preparation of Example 1412

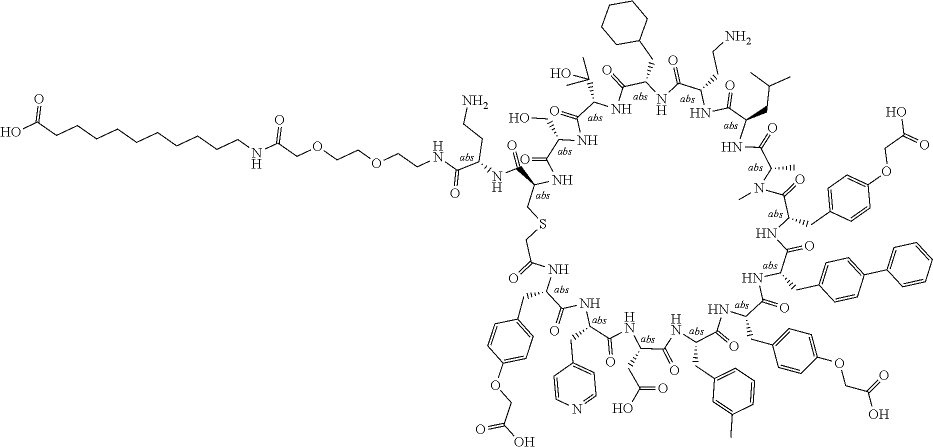 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1412 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 23% B, 23-63% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 17.2 mg, and its estimated purity by LCMS analysis was 99.5%. Analysis condition A: Retention time=1.53 min; ESI-MS(+) m/z [M+2H]²⁺: 1278.1.

### Preparation of Example 1413

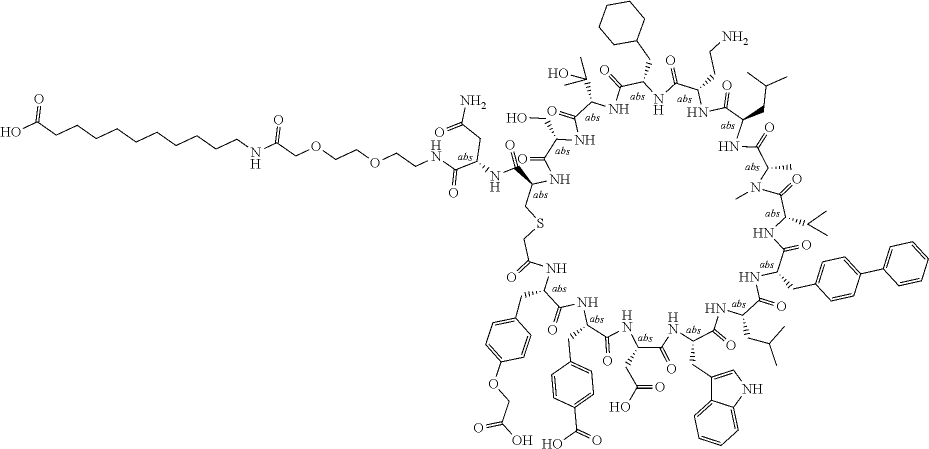 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1413 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 32% B, 32-72% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 47.2 mg, and its estimated purity by LCMS analysis was 93.2%. Analysis condition A: Retention time=1.83 min; ESI-MS(+) m/z [M+2H]²⁺: 1204.1.

### Preparation of Example 1414

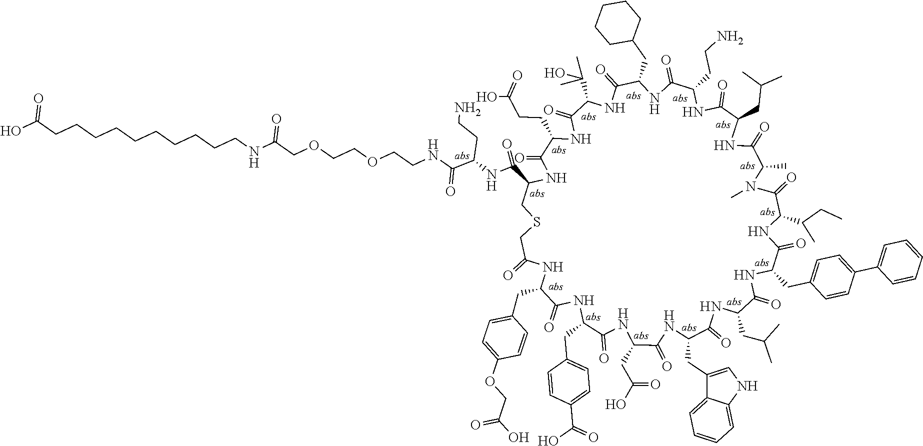 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 1414 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 30% B, 30-70% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33 mg, and its estimated purity by LCMS analysis was 97.4%. Analysis condition A: Retention time=1.68 min; ESI-MS(+) m/z [M+2H]²⁺: 1225.2.

### Preparation of Example 1415

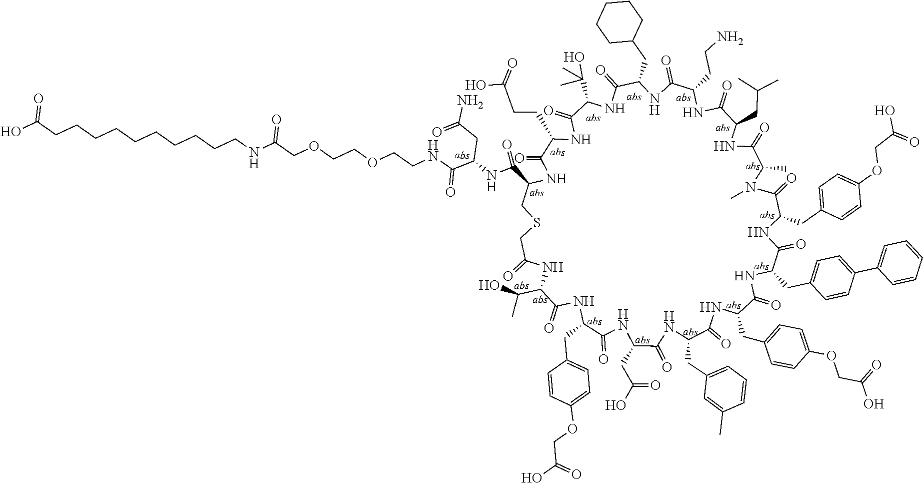 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CC(N)=O)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1415 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 10% B, 10-50% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 60.1 mg, and its estimated purity by LCMS analysis was 95.9%. Analysis condition B: Retention time=1.89 min; ESI-MS(+) m/z [M+2H]²⁺: 1283.

### Preparation of Example 1416

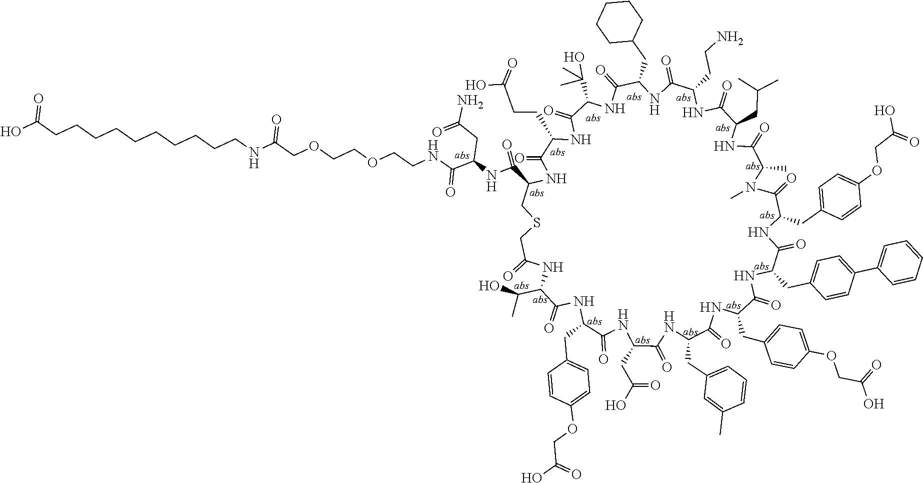 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@H](CC(N)=O)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1416 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 22 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 80 mg, and its estimated purity by LCMS analysis was 95.9%. Analysis condition A: Retention time=1.29 min; ESI-MS(+) m/z [M+2H]²⁺: 1282.1.

### Preparation of Example 1417

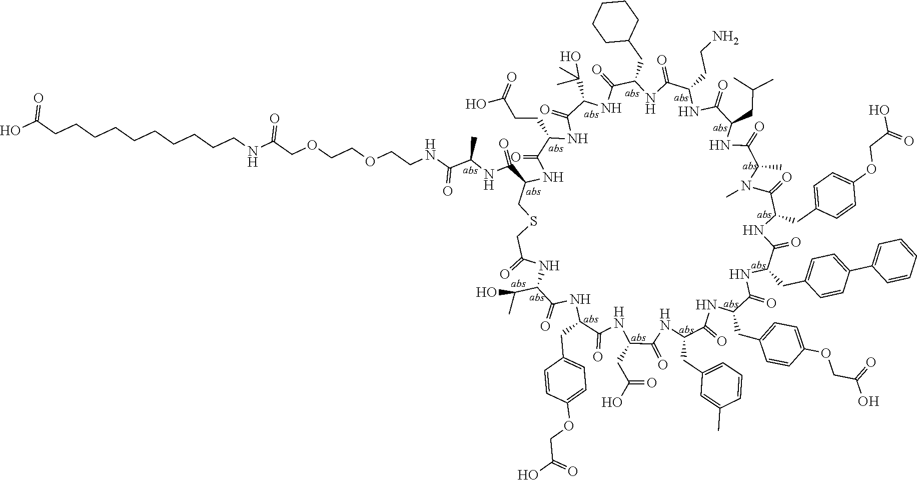 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@H](C)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1417 was prepared, using 2-Chlorotrityl resin pre-loaded with 11-((((9H-fluoren-9-yl)methoxy)carbonyl)amino)undecanoic acid on a 50 □mol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 47.3 mg, and its estimated purity by LCMS analysis was 100%. Analysis condition A: Retention time=1.42 min; ESI-MS(+) m/z [M+2H]²⁺: 1261.1.

### Preparation of Example 1418

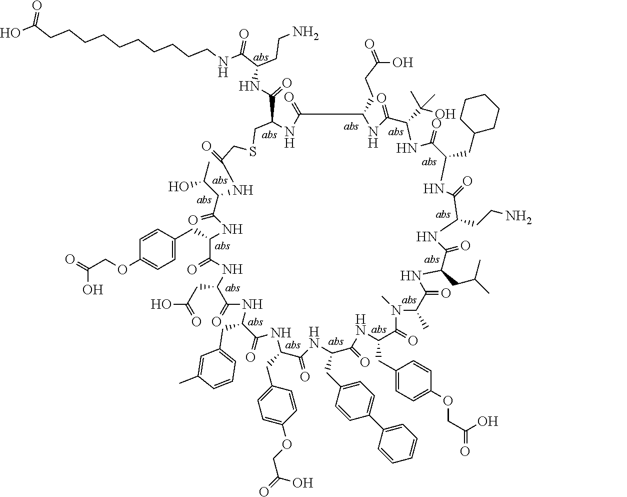 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1418 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 23% B, 23-63% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 71.3 mg, and its estimated purity by LCMS analysis was 94.5%. Analysis condition A: Retention time=1.37 min; ESI-MS(+) m/z [M+2H]²⁺: 1202.3.

### Preparation of Example 1419

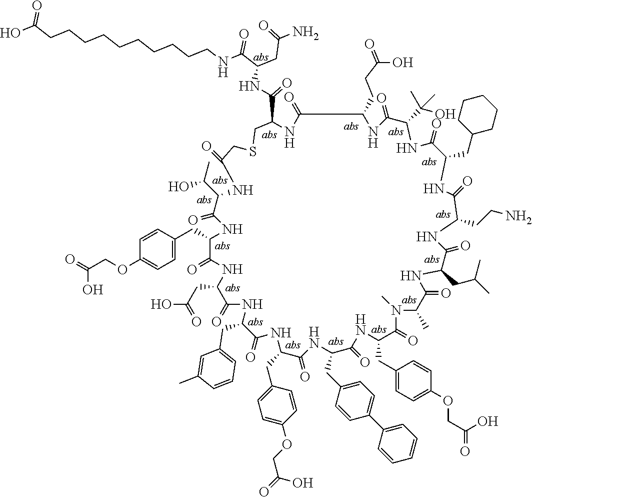 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CC(N)=O)C(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1419 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 10% B, 10-50% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 11% B, 11-51% B over 20 minutes, then a 1-minute hold at 100% B; Flow Rate: 40 mL/min; Column Temperature: 25 C. Fraction collection was triggered by MS signals. Fractions containing the desired product were combined and dried via centrifugal evaporation. The yield of the product was 8.8 mg, and its estimated purity by LCMS analysis was 100%. Analysis condition B: Retention time=1.95 min; ESI-MS(+) m/z [M+2H]²⁺: 1209.9.

### Preparation of Example 1420

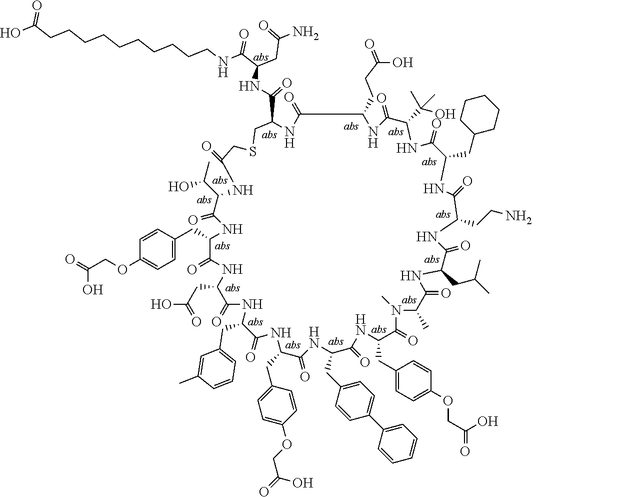 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@H](CC(N)=O)C(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1420 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 10% B, 10-50% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 69 mg, and its estimated purity by LCMS analysis was 100%. Analysis condition A: Retention time=1.38 min; ESI-MS(+) m/z [M+2H]²⁺: 1210.3.

### Preparation of Example 1421

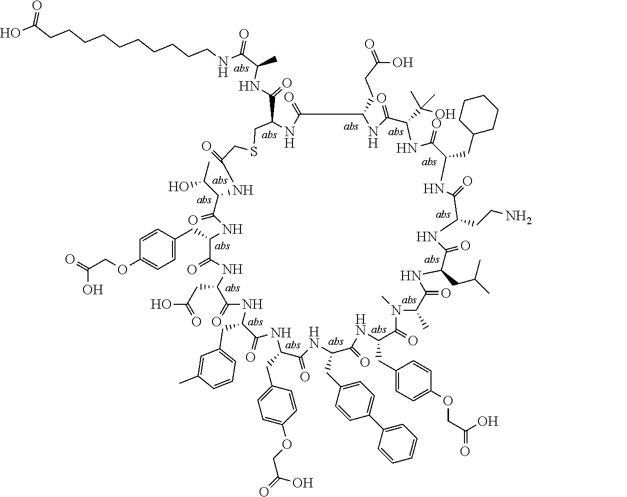 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@H](C)C(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1421 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 46.2 mg, and its estimated purity by LCMS analysis was 100%. Analysis condition A: Retention time=1.41 min; ESI-MS(+) m/z [M+2H]²⁺: 1189.1.

### Preparation of Example 1422

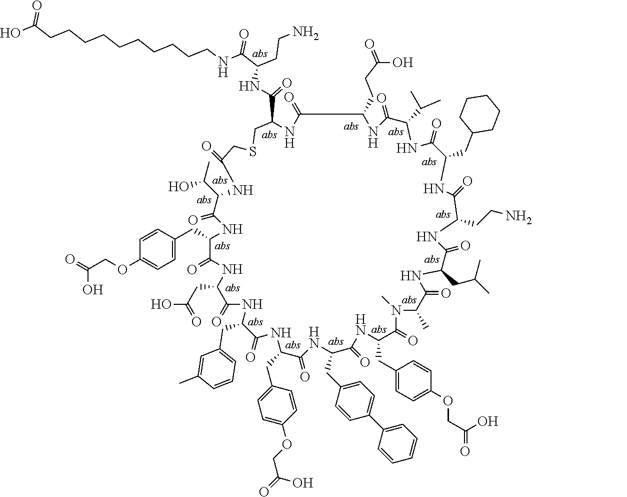 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1422 was prepared, using 2-Chlorotrityl resin pre-loaded with 11-((((9H-fluoren-9-yl)methoxy)carbonyl)amino)undecanoic acid on a 50 gmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 25.4 mg, and its estimated purity by LCMS analysis was 100%. Analysis condition A: Retention time=1.41 min; ESI-MS(+) m/z [M+2H]²⁺: 1195.2.

### Preparation of Example 1423

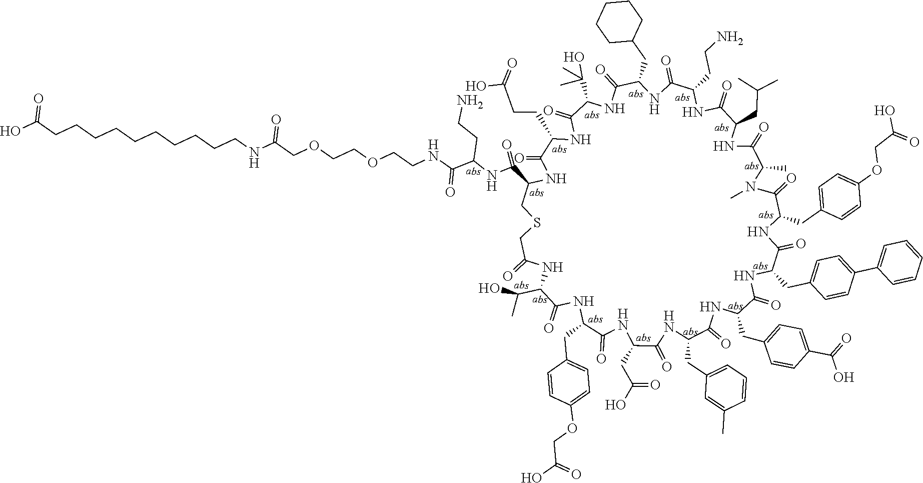 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)NC(CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 1423 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 57.9 mg, and its estimated purity by LCMS analysis was 95.9%. Analysis condition B: Retention time=1.9 min; ESI-MS(+) m/z [M+2H]²⁺: 1260.5.

### Preparation of Example 1424

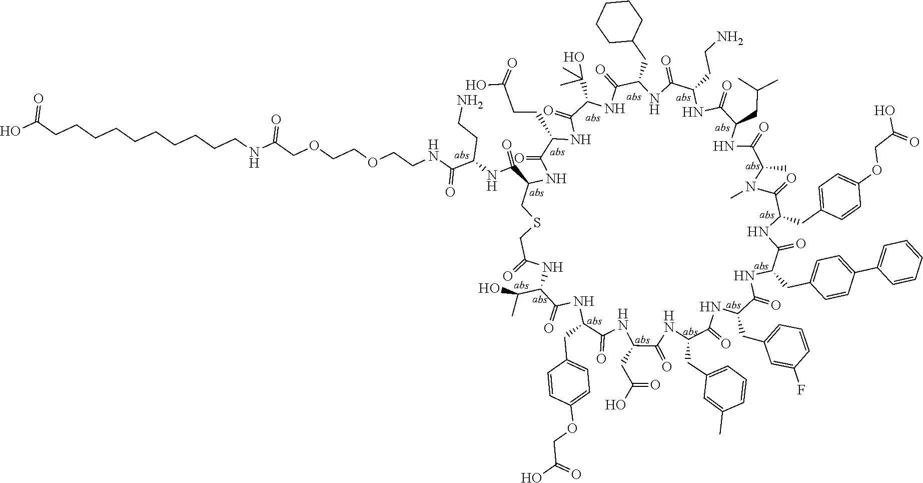 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Example 1424 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 17% B, 17-57% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 32.9 mg, and its estimated purity by LCMS analysis was 100%. Analysis condition A: Retention time=1.59 min; ESI-MS(+) m/z [M+2H]²⁺: 1247.

### Preparation of Example 1425

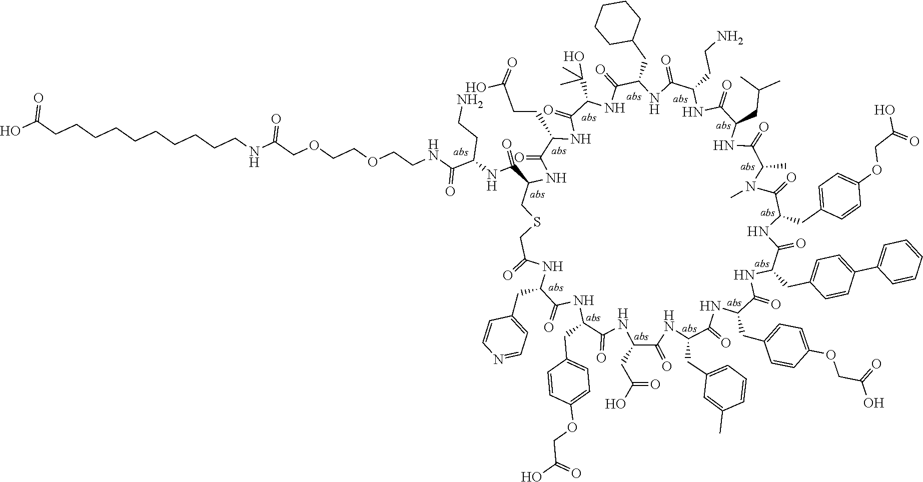 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1425 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.3 mg, and its estimated purity by LCMS analysis was 100%. Analysis condition A: Retention time=1.47 min; ESI-MS(+) m/z [M+2H]²⁺: 1299.

### Preparation of Example 1426

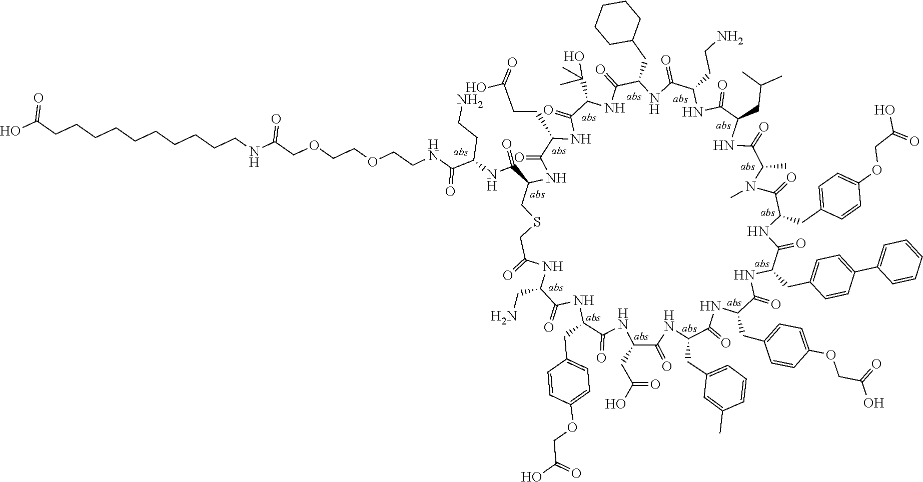 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1426 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 52.5 mg, and its estimated purity by LCMS analysis was 100%. Analysis condition A: Retention time=1.4 min; ESI-MS(+) m/z [M+2H]²⁺: 1268.4.

### Preparation of Example 1427

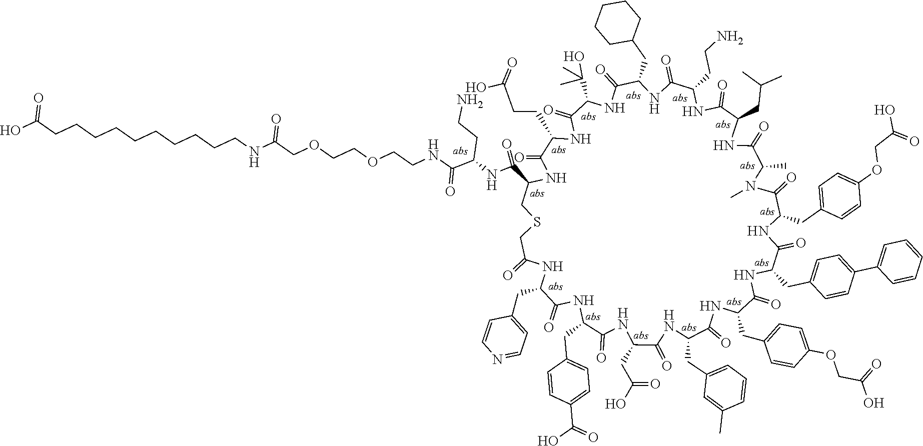 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1427 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 91.6 mg, and its estimated purity by LCMS analysis was 93.200. Analysis condition A: Retention time=min; ESI-MS(+) m/z [M+2H]²⁺: 1284.16, 1284.16.

### Preparation of Example 1428

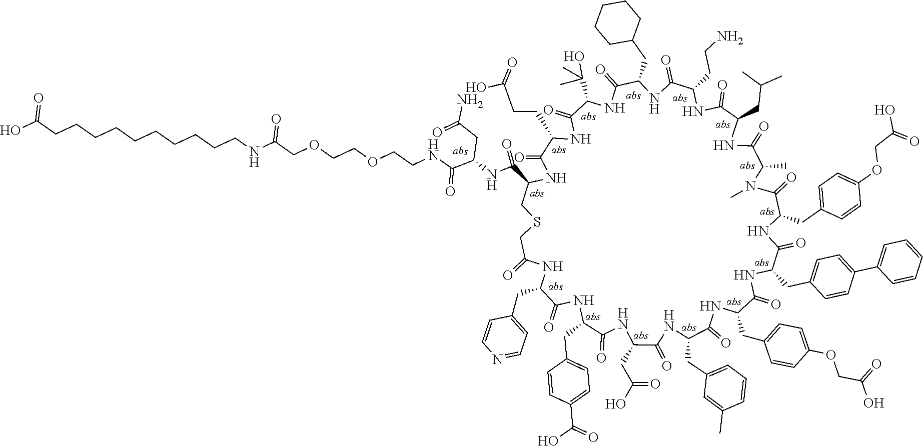 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1428 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 23% B, 23-63% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 89.4 mg, and its estimated purity by LCMS analysis was 100%. Analysis condition B: Retention time=1.84 min; ESI-MS(+) m/z [M+2H]²⁺: 1291.2.

### Preparation of Example 1429

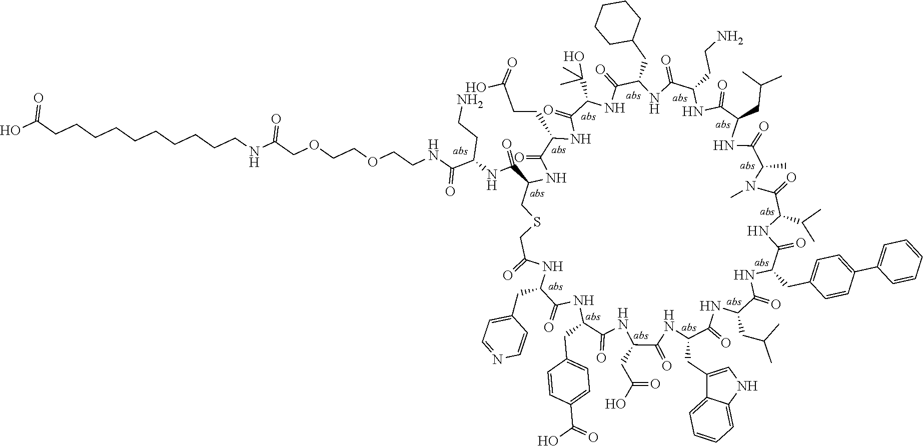 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 1429 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile: water with 10-mM ammonium acetate; Gradient: a 0-minute hold at 20% B, 20-60% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.7 mg, and its estimated purity by LCMS analysis was 100%. Analysis condition b: Retention time=1.71 min; ESI-MS(+) m/z [M+3H]³⁺: 788.

### Preparation of Example 1430

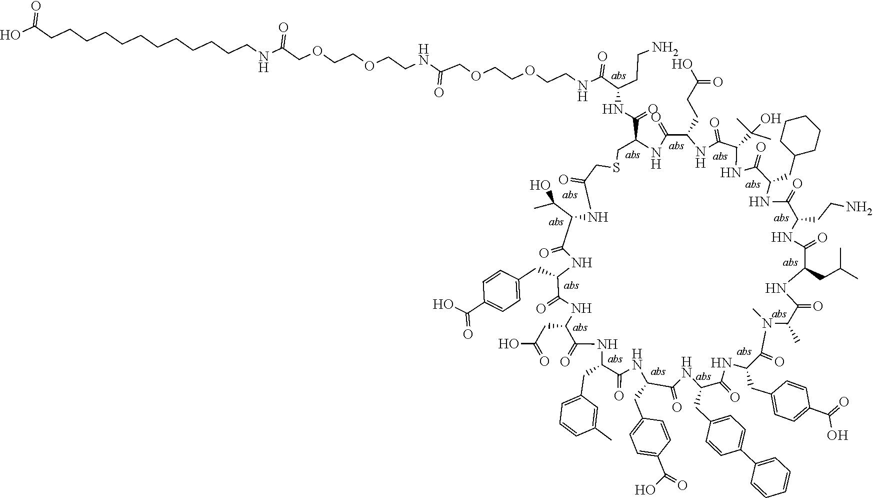 [SMILES: Cc1cccc(C[C@@H]2NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 1430 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 23% B, 23-63% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 54.4 mg, and its estimated purity by LCMS analysis was 96.2%. Analysis condition A: Retention time=1.45 min; ESI-MS(+) m/z [M+2H]²⁺: 1317.2.

### Preparation of Example 1431

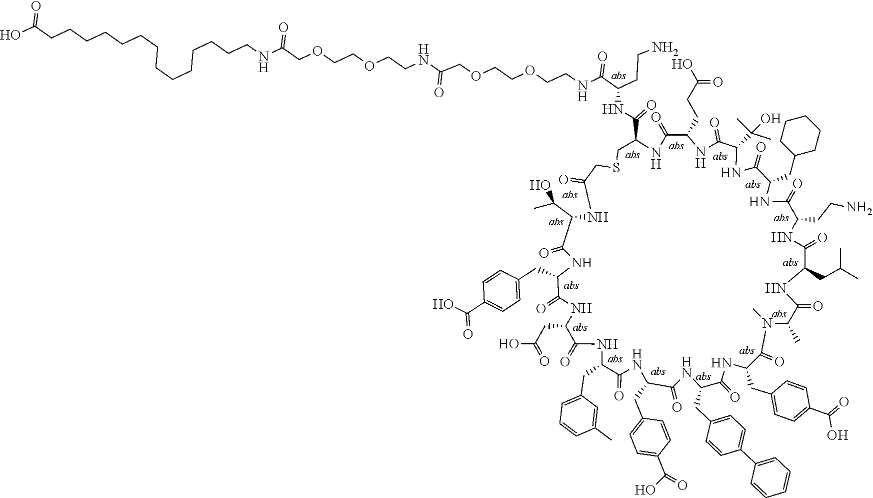 [SMILES: Cc1cccc(C[C@@H]2NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 1431 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 25% B, 25-65% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 65.5 mg, and its estimated purity by LCMS analysis was 94.4%. Analysis condition B: Retention time=1.91 min; ESI-MS(+) m/z [M+2H]²⁺: 1331.1.

### Preparation of Example 1432

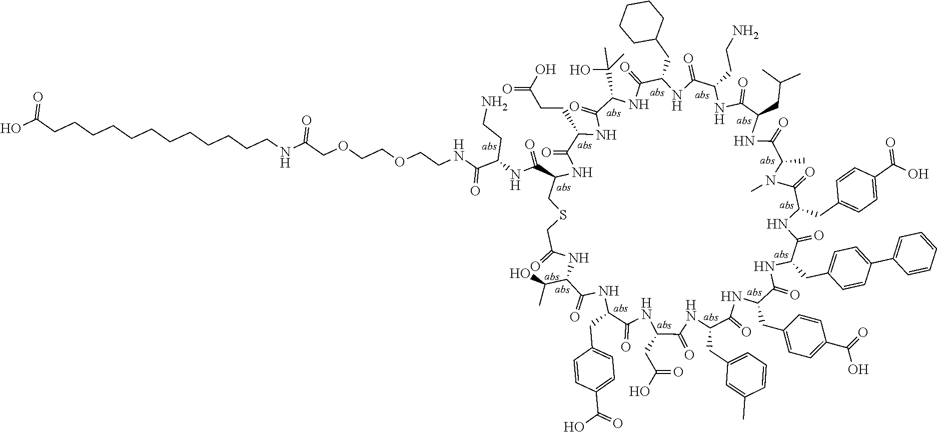 [SMILES: Cc1cccc(C[C@@H]2NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 1432 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 44.3 mg, and its estimated purity by LCMS analysis was 92.2%. Analysis condition B: Retention time=1.86 min; ESI-MS(+) m/z [M+2H]²⁺: 1245.1.

### Preparation of Example 1433

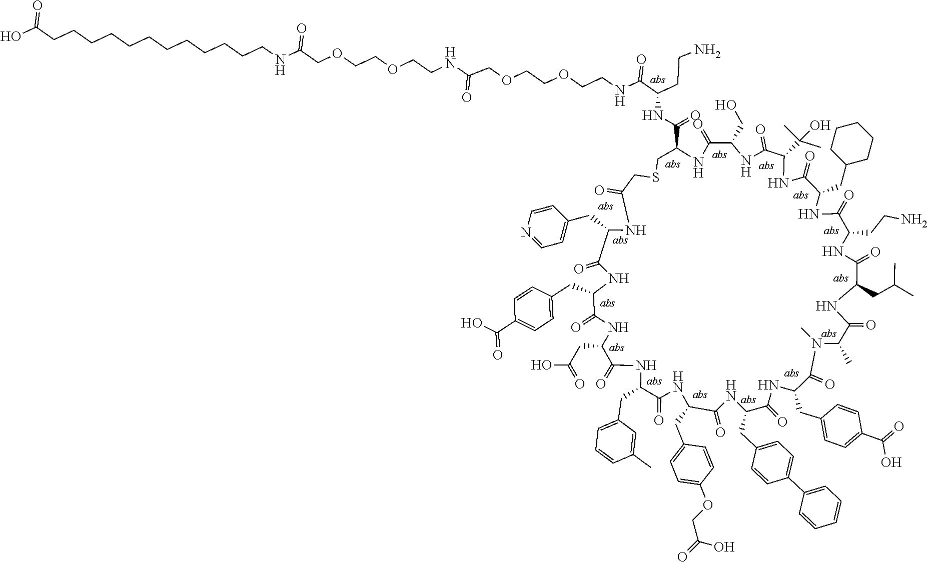 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Example 1433 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 20% B, 20-60% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 56.2 mg, and its estimated purity by LCMS analysis was 95.4%. Analysis condition B: Retention time=1.7 min; ESI-MS(+) m/z [M+2H]²⁺: 1349.9.

### Preparation of Example 1434

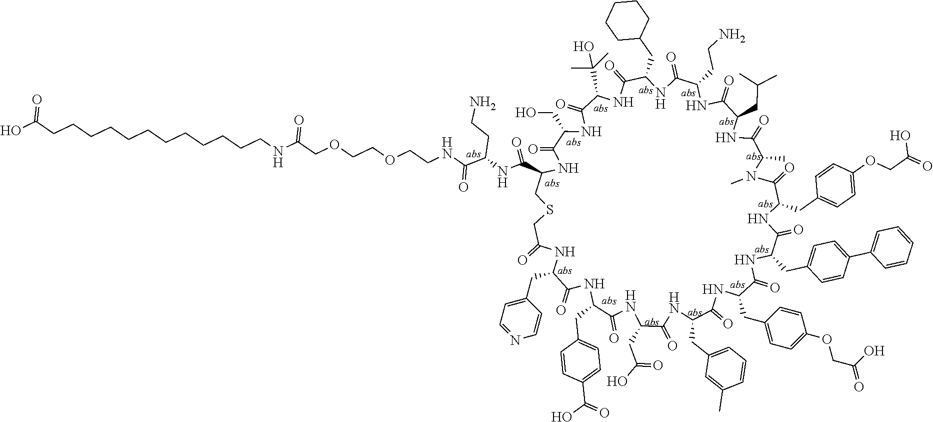 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 1434 was prepared, using 2-Chlorotrityl resin pre-loaded with 11-((((9H-fluoren-9-yl)methoxy)carbonyl)amino)undecanoic acid on a 50 μmol scale, following the general synthetic sequence described for the preparation of Example 1000 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile: water with 0.05% trifluoroacetic acid; Gradient: a 0-minute hold at 18% B, 18-58% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33.3 mg, and its estimated purity by LCMS analysis was 97.1%. Analysis condition A: Retention time=1.52 min; ESI-MS(+) m/z [M+2H]²⁺: 1277.1.

### Preparation of Example 2000

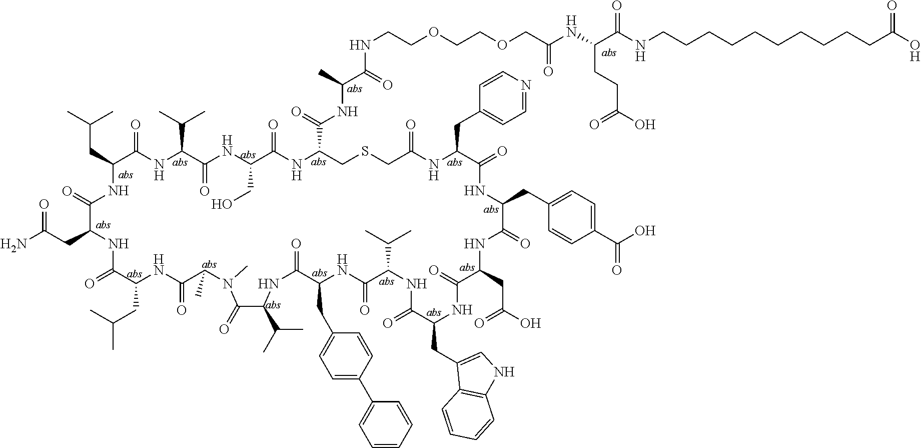 [SMILES: CC(C)C[C@@H]1NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(=O)NCCOCCOCC(=O)N[C@@H](CCC(=O)O)C(=O)NCCCCCCCCCCC(=O)O)NC(=O)[C@H](CO)NC(=O)[C@H](C(C)C)NC1=O]

Example 2000 was prepared on a 50 μmol scale. The yield of the product was 9.8 mg, and its estimated purity by LCMS analysis was 98.8%. Analysis condition B: Retention time=1.96 min; ESI-MS(+) m/z [M+2H]²⁺: 1189.9.

### Preparation of Example 2001

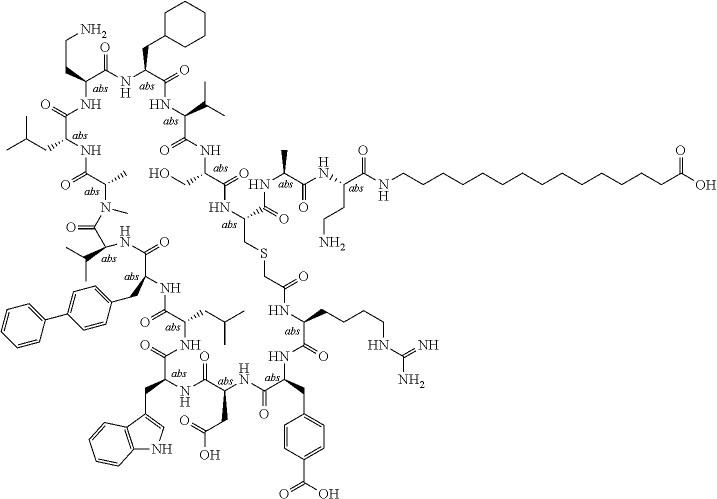 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCCCNC(=N)N)NC(=O)CSC[C@@H](C(=O)N[C@@H](C)C(=O)N[C@@H](CCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2001 was prepared on a 25 μmol scale. The yield of the product was 7.4 mg, and its estimated purity by LCMS analysis was 97.9%. Analysis condition B: Retention time=1.96 min; ESI-MS(+) m/z [M+3H]³⁺: 769.9.

### Preparation of Example 2002

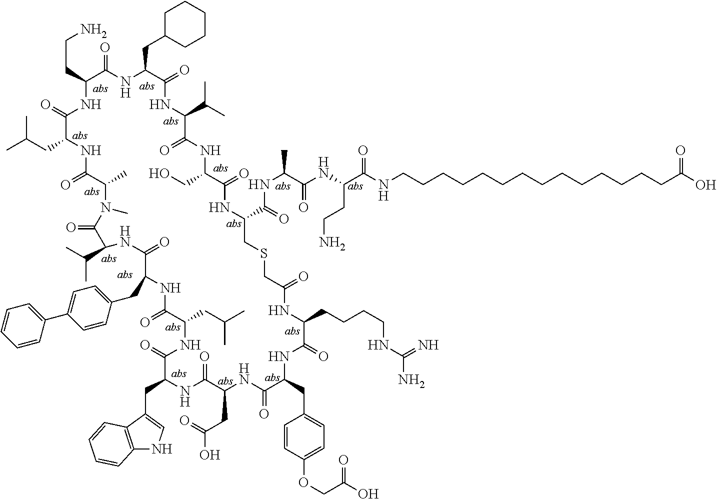 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(=O)N[C@@H](CCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2002 was prepared on a 25 μmol scale. The yield of the product was 11.1 mg, and its estimated purity by LCMS analysis was 95.7%. Analysis condition A: Retention time=1.98 min; ESI-MS(+) m/z [M+3H]³⁺: 780.2.

### Preparation of Example 2003

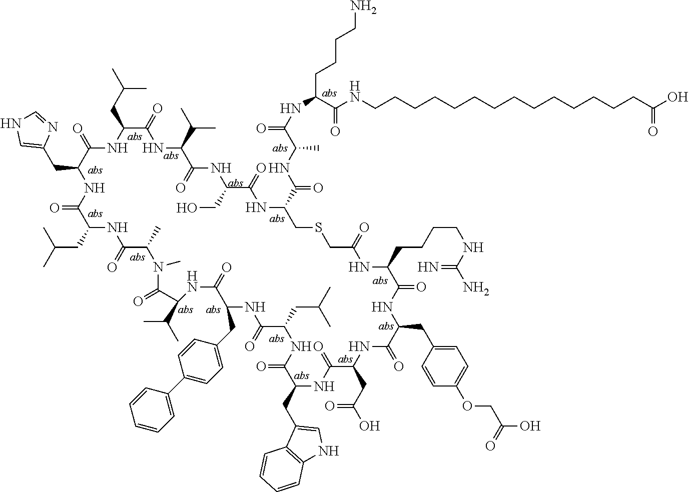 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(=O)N[C@@H](CCCCN)C(=O)NCCCCCCCCCCCCCCC(=O)O)NC(=O)[C@H](CO)NC(=O)[C@H](C(C)C)NC(=O)[C@H](CC(C)C)NC(=O)[C@H](Cc2c[nH]cn2)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2003 was prepared on a 25 μmol scale. The yield of the product was 9.5 mg, and its estimated purity by LCMS analysis was 97.3%. Analysis condition B: Retention time=1.87 min; ESI-MS(+) m/z [M+2H]²⁺: 1182.1.

### Preparation of Example 2004

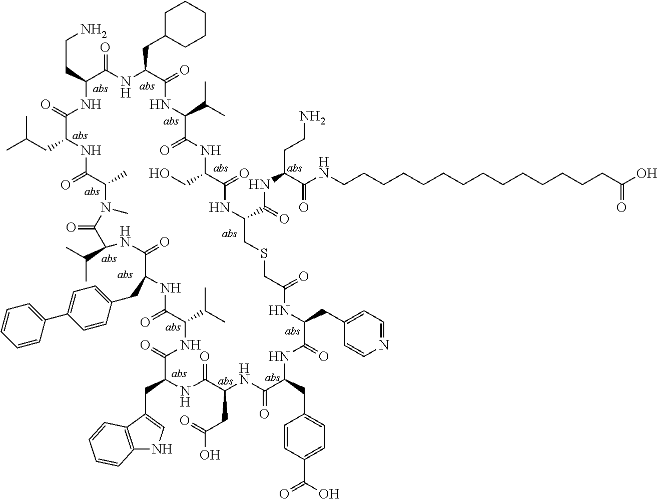 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2004 was prepared on a 25 μmol scale. The yield of the product was 12.1 mg, and its estimated purity by LCMS analysis was 96.6%. Analysis condition A: Retention time=1.7 min; ESI-MS(+) m/z [M+2H]²⁺: 1102.3.

### Preparation of Example 2005

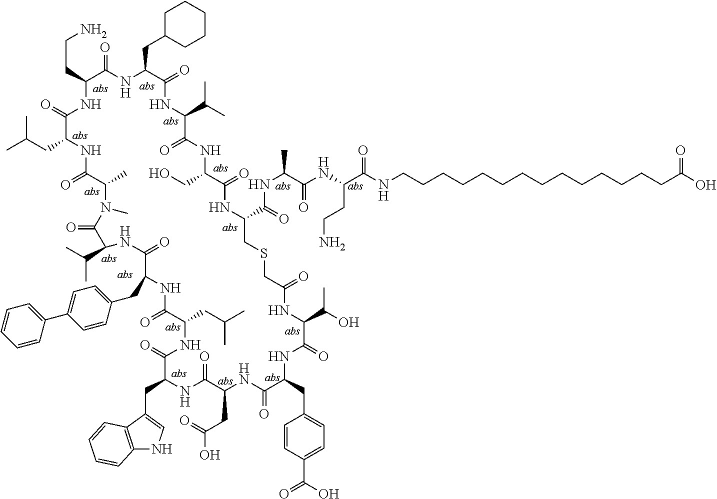 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](C)C(=O)N[C@@H](CCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2005 was prepared on a 25 μmol scale. The yield of the product was 9.8 mg, and its estimated purity by LCMS analysis was 95.3%. Analysis condition A: Retention time=2.02 min; ESI-MS(+) m/z [M+2H]²⁺: 1120.

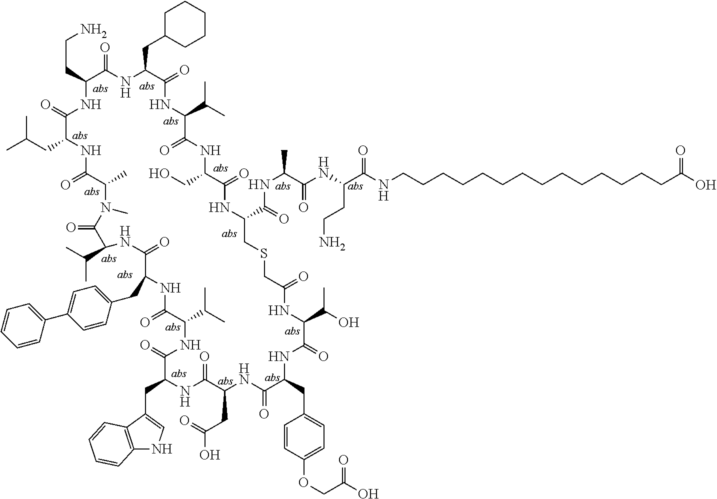 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](C)C(=O)N[C@@H](CCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2006 was prepared on a 25 μmol scale. The yield of the product was 8.3 mg, and its estimated purity by LCMS analysis was 95.2%. Analysis condition A: Retention time=2 min; ESI-MS(+) m/z [M+2H]²⁺: 1135.

### Preparation of Example 2007

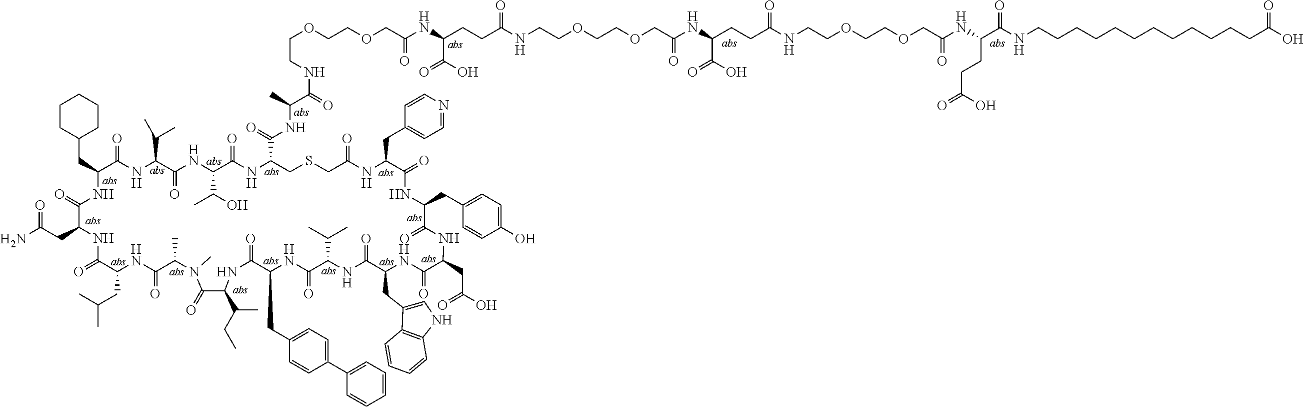 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(=O)NCCOCCOCC(=O)N[C@@H](CCC(=O)NCCOCCOCC(=O)N[C@@H](CCC(=O)NCCOCCOCC(=O)N[C@@H](CCC(=O)O)C(=O)NCCCCCCCCCCCCC(=O)O)C(=O)O)C(=O)O)NC(=O)[C@H](C(C)O)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2007 was prepared on a 50 μmol scale. The yield of the product was 84.7 mg, and its estimated purity by LCMS analysis was 95.1%. Analysis condition A: Retention time=1.53 min; ESI-MS(+) m/z [M+3H]³⁺: 985.

### Preparation of Example 2008

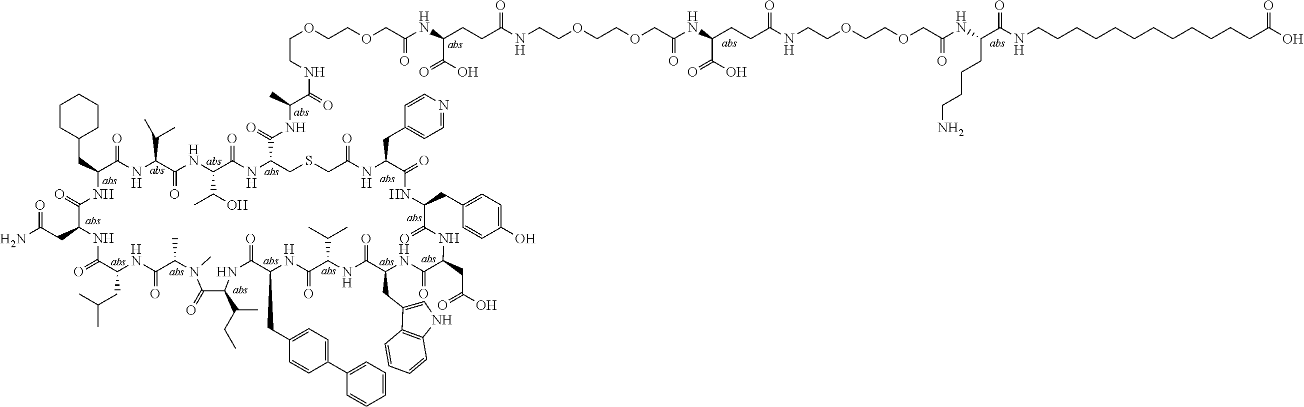 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(=O)NCCOCCOCC(=O)N[C@@H](CCC(=O)NCCOCCOCC(=O)N[C@@H](CCC(=O)NCCOCCOCC(=O)N[C@@H](CCCCN)C(=O)NCCCCCCCCCCCCC(=O)O)C(=O)O)C(=O)O)NC(=O)[C@H](C(C)O)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2008 was prepared on a 50 μmol scale. The yield of the product was 27.7 mg, and its estimated purity by LCMS analysis was 97.3%. Analysis condition B: Retention time=1.82 min; ESI-MS(+) m/z [M+2H]²⁺: 1475.9.

### Preparation of Example 2009

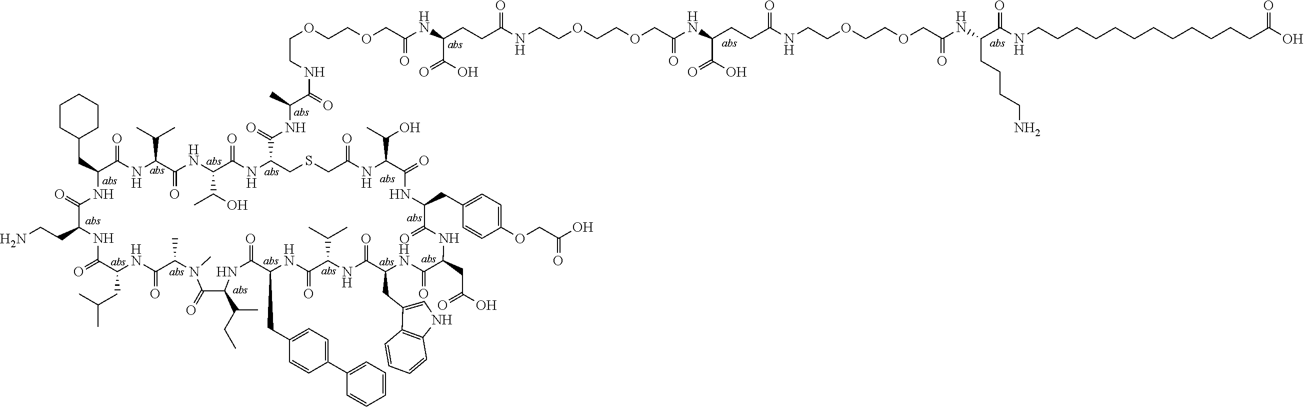 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](C)C(=O)NCCOCCOCC(=O)N[C@@H](CCC(=O)NCCOCCOCC(=O)N[C@@H](CCC(=O)NCCOCCOCC(=O)N[C@@H](CCCCN)C(=O)NCCCCCCCCCCCCC(=O)O)C(=O)O)C(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2009 was prepared on a 50 μmol scale. The yield of the product was 36.9 mg, and its estimated purity by LCMS analysis was 96.9%. Analysis condition B: Retention time=1.92, 2.11 min; ESI-MS(+) m/z [M+3H]³⁺: 993.2.

### Preparation of Example 2010

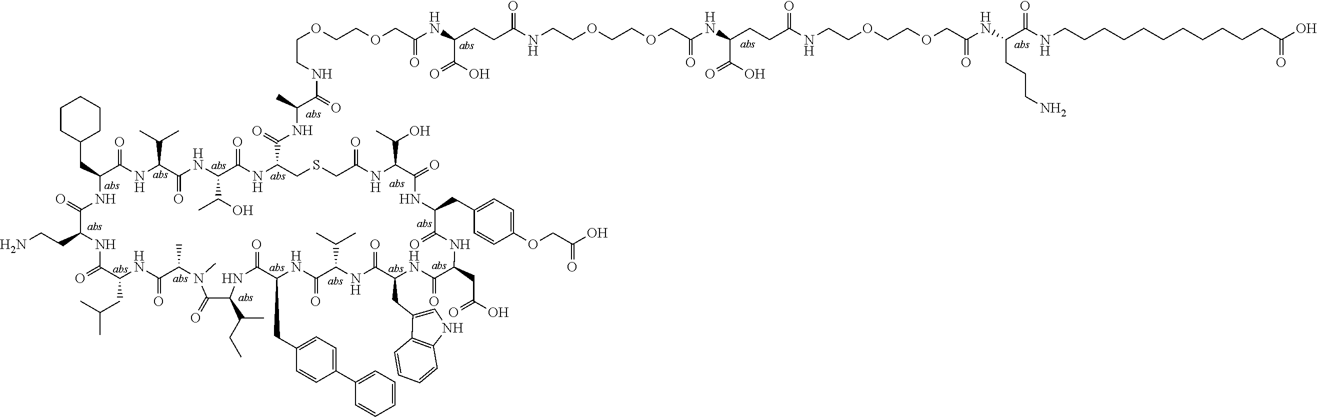 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](C)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCCC(=O)O)C(=O)O)C(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2010 was prepared on a 50 μmol scale. The yield of the product was 43.4 mg, and its estimated purity by LCMS analysis was 96.5%. Analysis condition A: Retention time=1.69 min; ESI-MS(+) m/z [M+2H]²⁺: 1475.1.

### Preparation of Example 2011

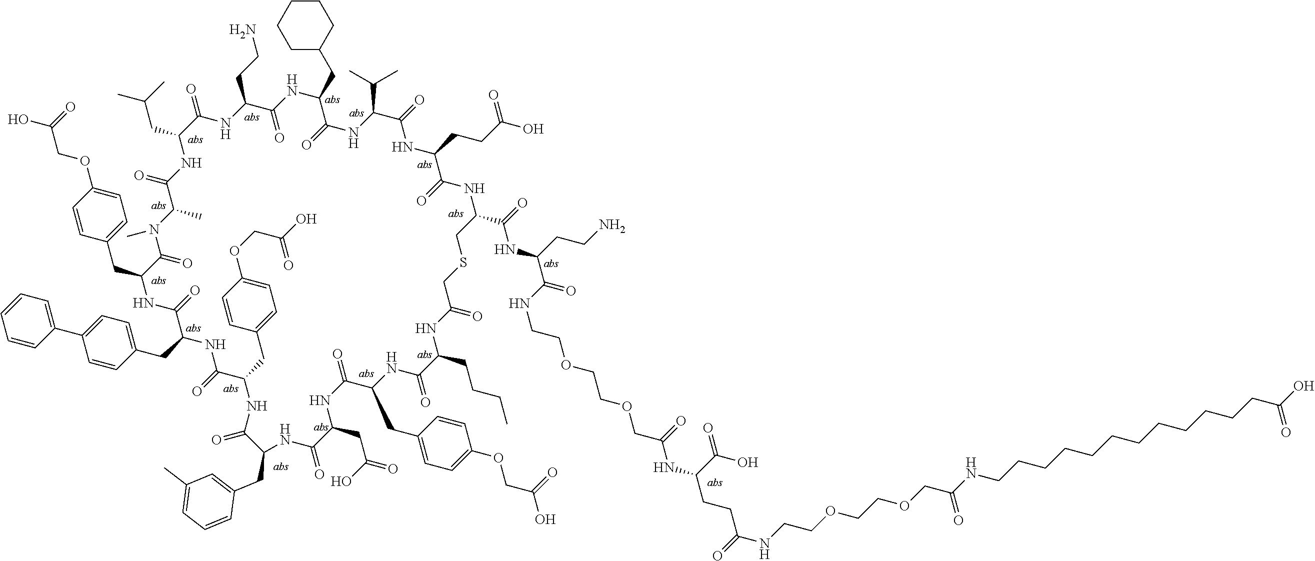 [SMILES: CCCC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Example 2011 was prepared on a 50 μmol scale. The yield of the product was 20.1 mg, and its estimated purity by LCMS analysis was 96.50%. Analysis condition B: Retention time=2.08 min; ESI-MS(+) m/z [M+2H]²⁺: 1423.9.

### Preparation of Example 2012

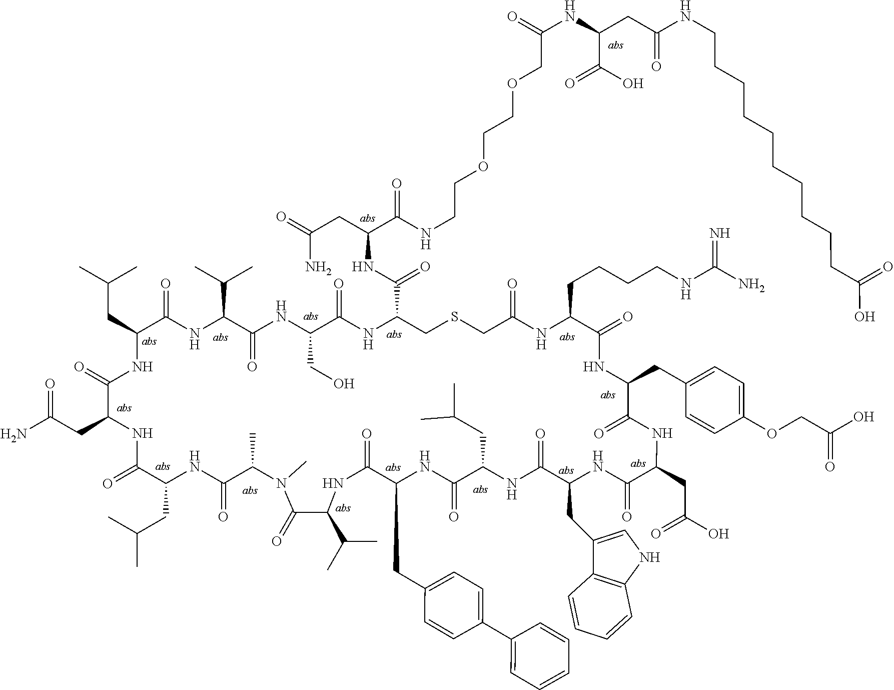 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(=O)NCCOCCOCC(=O)N[C@@H](CC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2012 was prepared on a 50 μmol scale. The yield of the product was 6.6 mg, and its estimated purity by LCMS analysis was 95.2%. Analysis condition A: Retention time=1.58 min; ESI-MS(+) m/z [M+2H]²⁺: 1230.1.

### Preparation of Example 2013

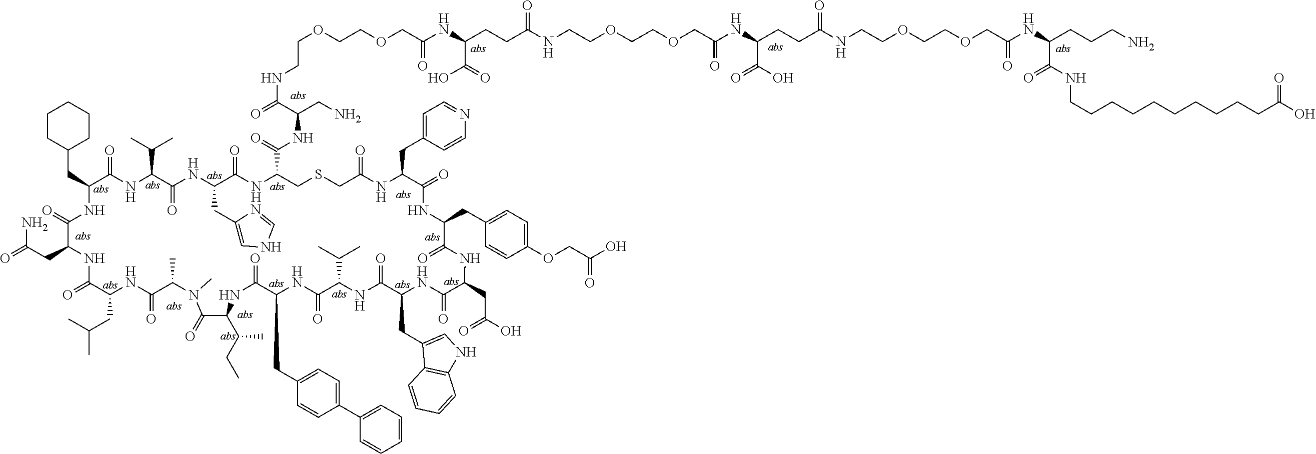 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CN)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](Cc2c[nH]cn2)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2013 was prepared on a 50 μmol scale. The yield of the product was 14.3 mg, and its estimated purity by LCMS analysis was 96.8%. Analysis condition B: Retention time=1.73 min; ESI-MS(+) m/z [M+3H]³⁺: 1016.3.

### Preparation of Example 2014

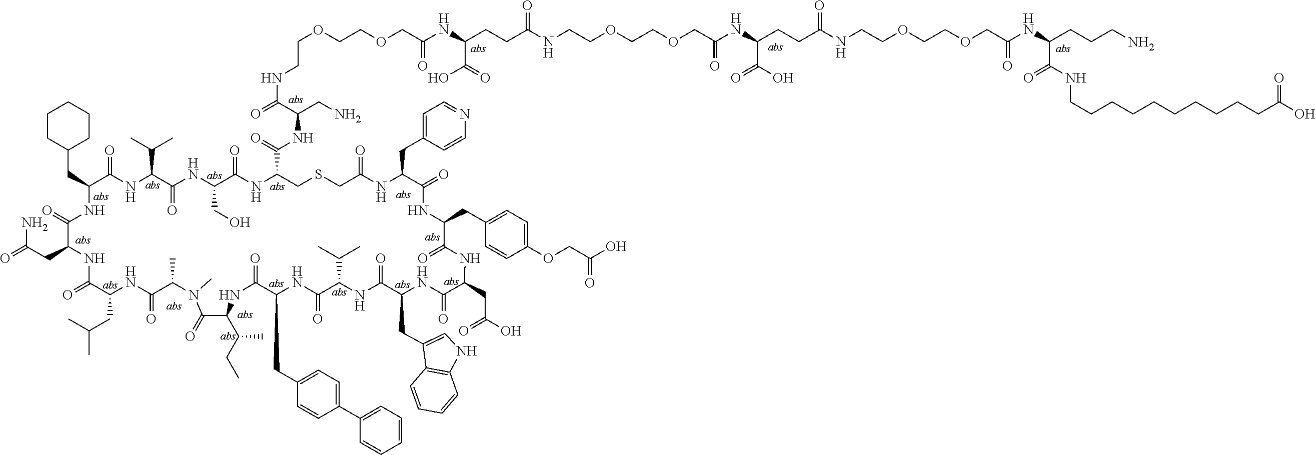 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CN)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2014 was prepared on a 50 μmol scale. The yield of the product was 70.3 mg, and its estimated purity by LCMS analysis was 95.2%. Analysis condition A: Retention time=1.53 min; ESI-MS(+) m/z [M+3H]³⁺: 1000.

### Preparation of Example 2015

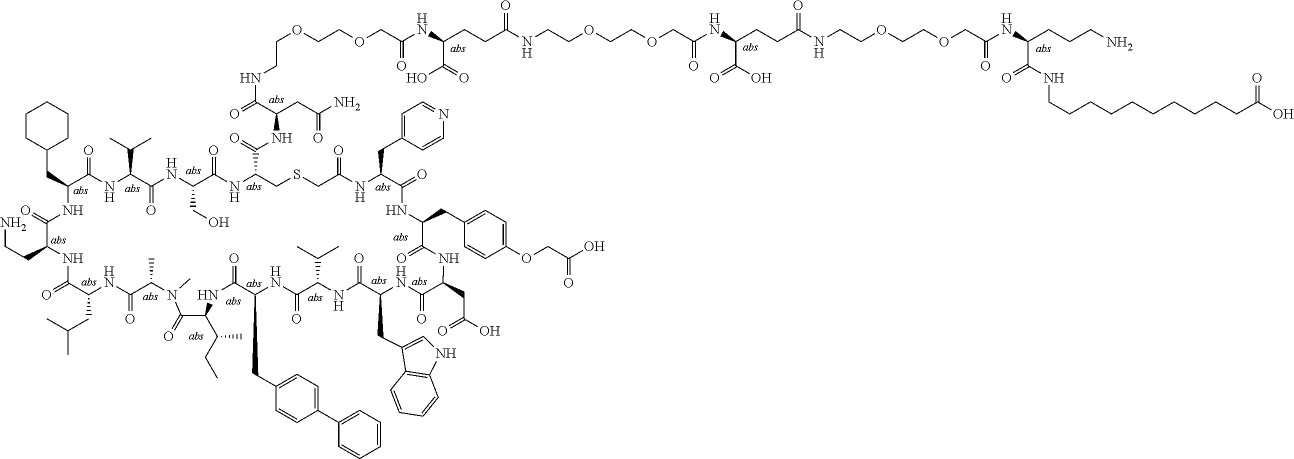 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2015 was prepared on a 50 μmol scale. The yield of the product was 51.3 mg, and its estimated purity by LCMS analysis was 100%. Analysis condition A: Retention time=1.61 min; ESI-MS(+) m/z [M+3H]³⁺: 1004.1.

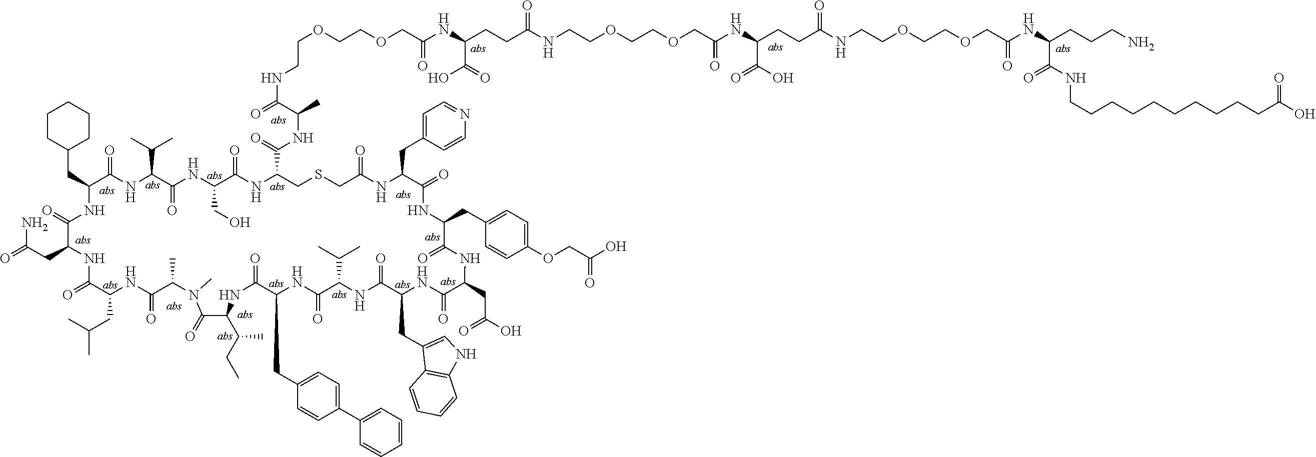 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](C)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2016 was prepared on a 50 μmol scale. The yield of the product was 14.6 mg, and its estimated purity by LCMS analysis was 100%. Analysis condition A: Retention time=1.55 min; ESI-MS(+) m/z [M+2H]²⁺: 1491.2.

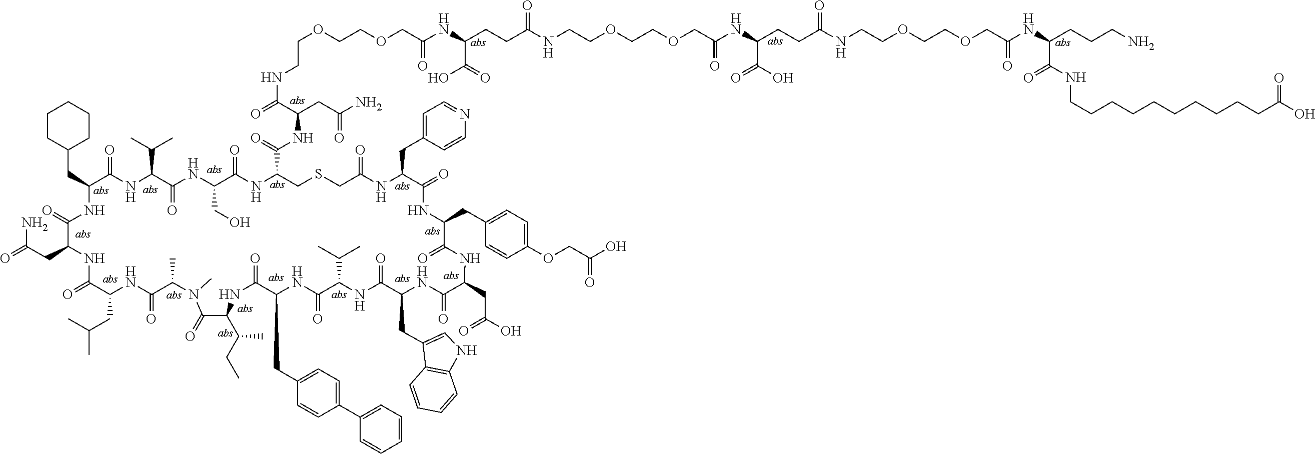 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2017 was prepared on a 50 μmol scale. The yield of the product was 35.7 mg, and its estimated purity by LCMS analysis was 100%. Analysis condition B: Retention time=1.8 min; ESI-MS(+) m/z [M+3H]³⁺: 1009.2.

### Preparation of Example 2018

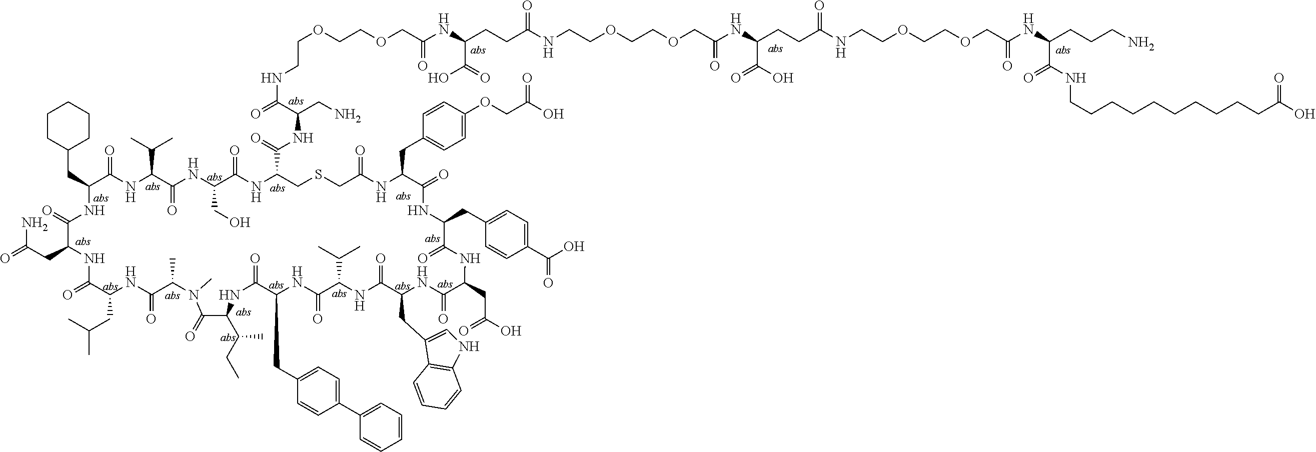 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CN)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2018 was prepared on a 50 μmol scale. The yield of the product was 26.2 mg, and its estimated purity by L CMS analysis was 100%. Analysis condition A: Retention time=1.65 min; ESI-MS(+) m/z [M+2H]²⁺: 1520.1.

### Preparation of Example 2019

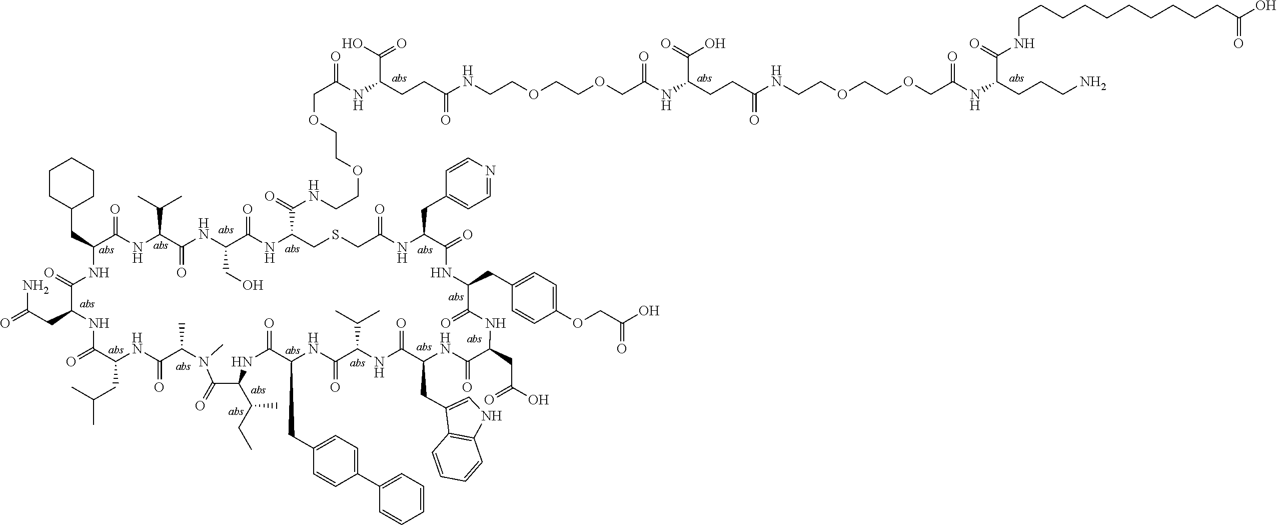 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2019 was prepared on a 50 μmol scale. The yield of the product was 71.1 mg, and its estimated purity by LCMS analysis was 100%. Analysis condition B: Retention time=1.82 min; ESI-MS(+) m/z [M+3H]³⁺: 971.

### Preparation of Example 2020

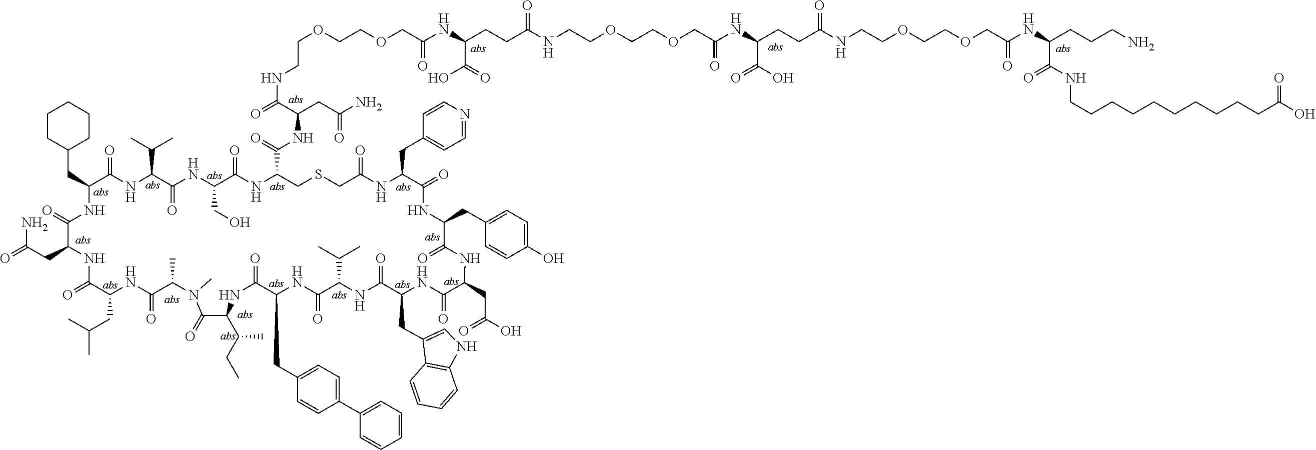 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2020 was prepared on a 50 μmol scale. The yield of the product was 22.2 mg, and its estimated purity by LCMS analysis was 100%. Analysis condition A: Retention time=1.72 min; ESI-MS(+) m/z [M+2H]²⁺: 1484.1.

### Preparation of Example 2021

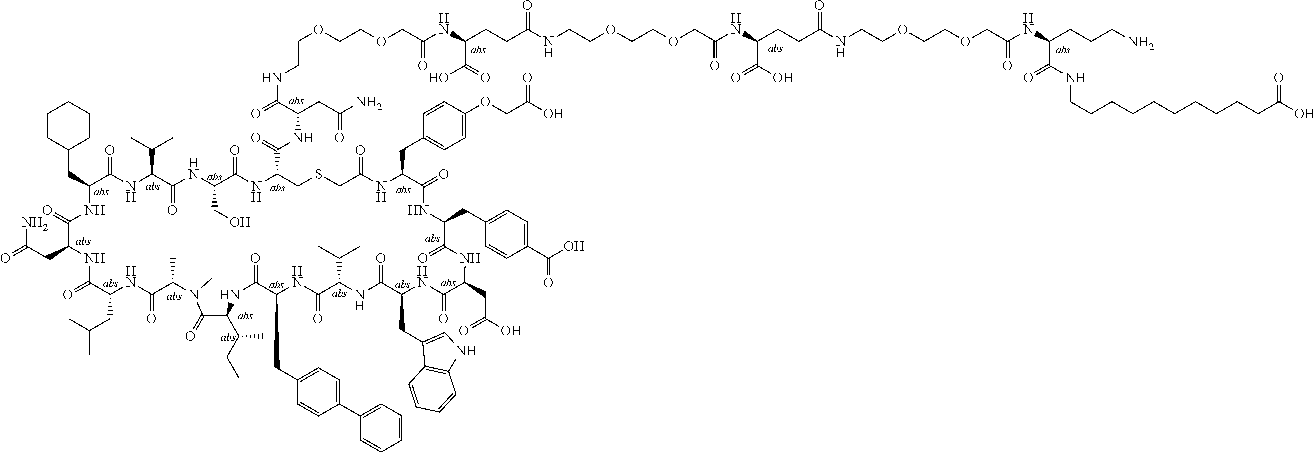 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2021 was prepared on a 50 μmol scale. The yield of the product was 27.2 mg, and its estimated purity by LCMS analysis was 95%. Analysis condition A: Retention time=1.92 min; ESI-MS(+) m/z [M+2H]²⁺: 1534.3.

### Preparation of Example 2022

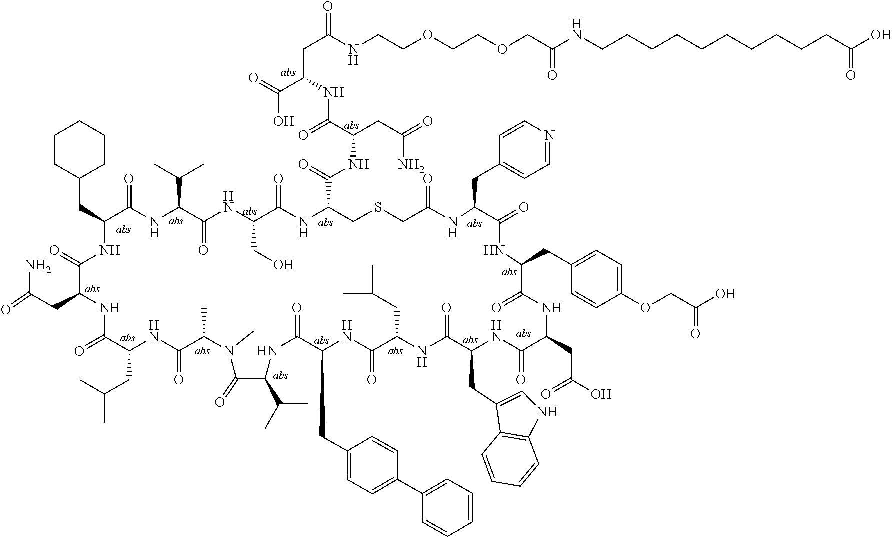 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)N[C@@H](CC(=O)NCCOCCOCC(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2022 was prepared on a 50 μmol scale. The yield of the product was 36.4 mg, and its estimated purity by LCMS analysis was 100%. Analysis condition A: Retention time=1.67 min; ESI-MS(+) m/z [M+2H]²⁺: 1239.1.

### Preparation of Example 2023

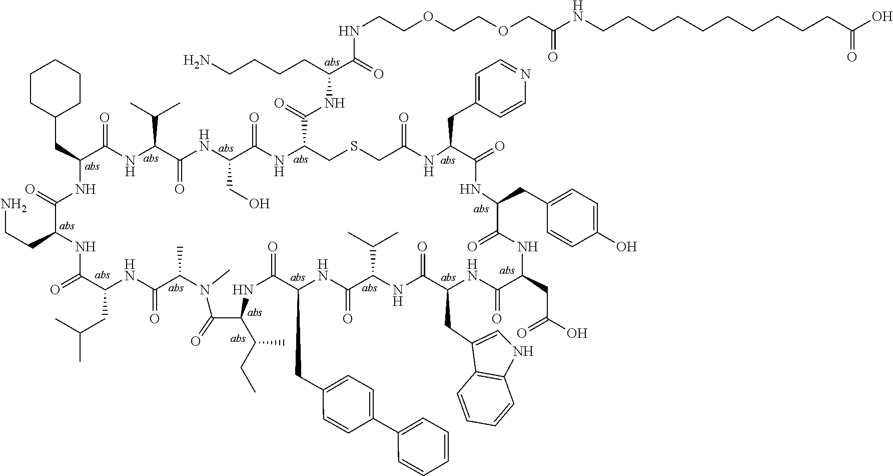 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2023 was prepared on a 50 μmol scale. The yield of the product was 22.1 mg, and its estimated purity by LCMS analysis was 98.8%. Analysis condition B: Retention time=1.76 min; ESI-MS(+) m/z [M+3H]³⁺: 769.

### Preparation of Example 2024

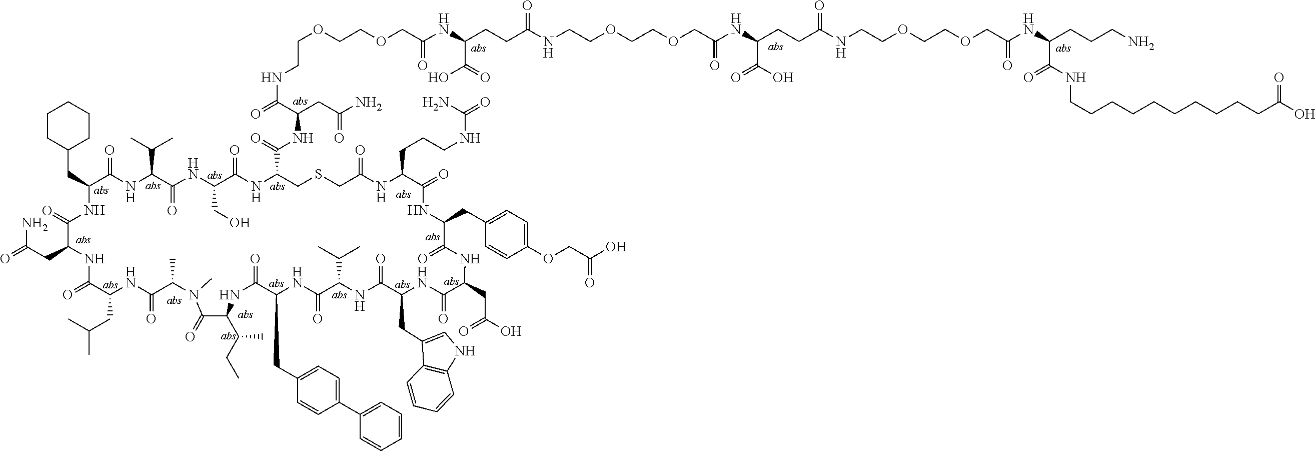 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(N)=O)NC(=O)CSC[C@@H](C(=O)N[C@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2024 was prepared on a 50 μmol scale. The yield of the product was 12.4 mg, and its estimated purity by LCMS analysis was 96.7%. Analysis condition A: Retention time=1.52 min; ESI-MS(+) m/z [M+3H]³⁺: 1011.9.

### Preparation of Example 2025

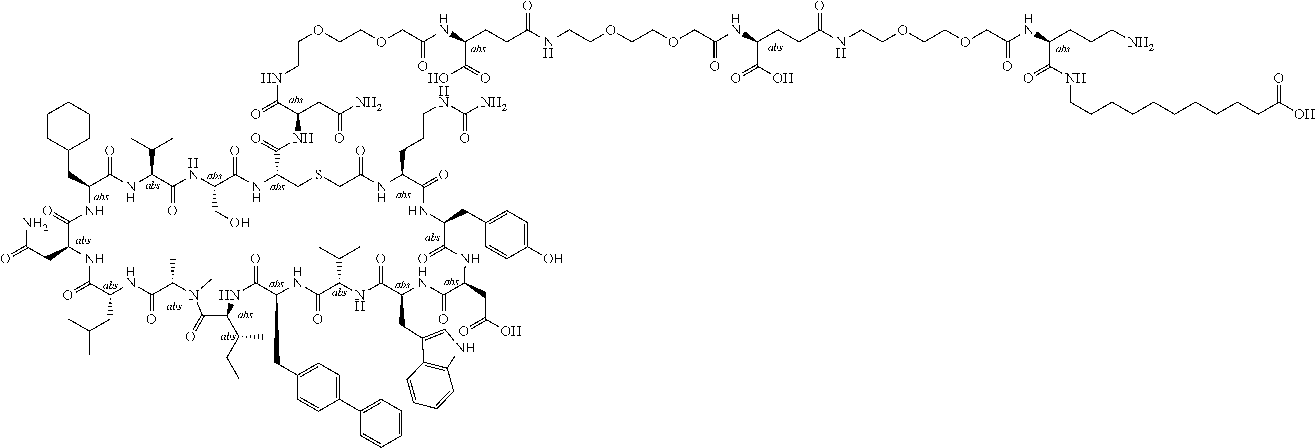 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCNC(N)=O)NC(=O)CSC[C@@H](C(=O)N[C@H](CC(N)=O)C(=O)NCCOCCOCC(=O)N[C@@H](CCC(=O)NCCOCCOCC(=O)N[C@@H](CCC(=O)NCCOCCOCC(=O)N[C@@H](CCCN)C(=O)NCCCCCCCCCCC(=O)O)C(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2025 was prepared on a 50 μmol scale. The yield of the product was 6.4 mg, and its estimated purity by LCMS analysis was 100%. Analysis condition A: Retention time=1.64 min; ESI-MS(+) m/z [M+3H]³⁺: 992.2.

### Preparation of Example 2026

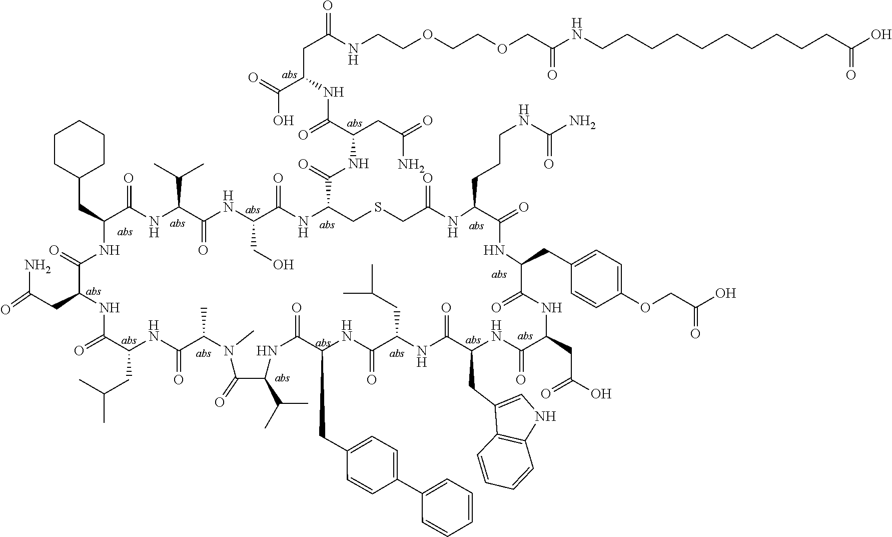 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(N)=O)NC(=O)CSC[C@@H](C(=O)N[C@@H](CC(N)=O)C(=O)N[C@@H](CC(=O)NCCOCCOCC(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2026 was prepared on a 50 μmol scale. The yield of the product was 7.7 mg, and its estimated purity by LCMS analysis was 95.1%. Analysis condition B: Retention time=1.75 min; ESI-MS(+) m/z [M+2H]²⁺: 1243.27, 1243.32.

### Preparation of Example 2027

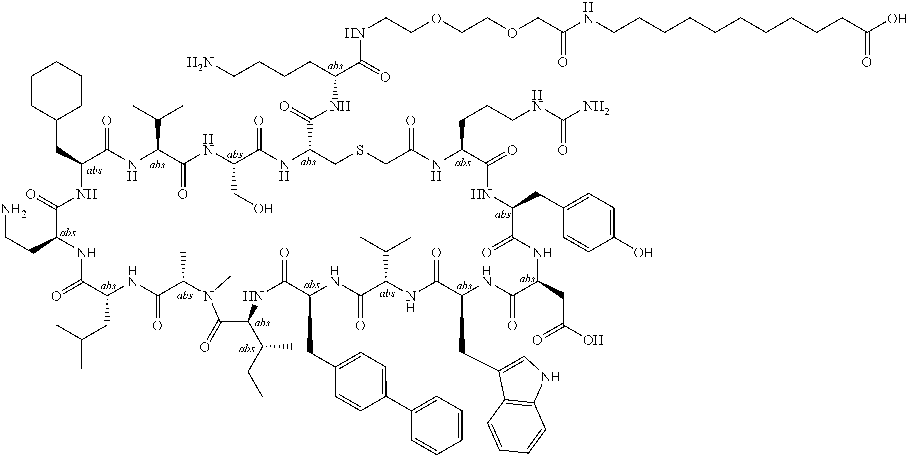

Example 2027 was prepared on a 50 μmol scale. The yield of the product was 20.3 mg, and its estimated purity by LCMS analysis was 100%. Analysis condition A: Retention time=1.67 min; ESI-MS(+) m/z [M+2H]²⁺: 1157.1.

### Preparation of Example 2028

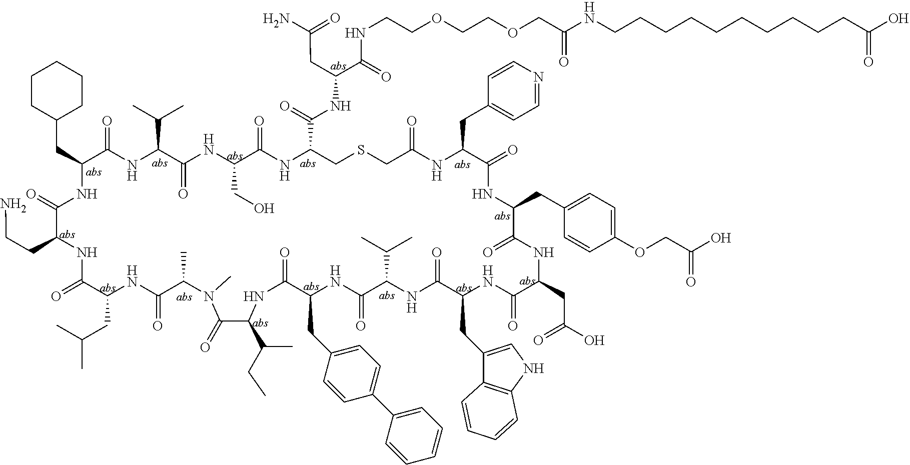

Example 2028 was prepared on a 25 μmol scale. The yield of the product was 27.7 mg, and its estimated purity by LCMS analysis was 97.1%. Analysis condition A: Retention time=1.72 min; ESI-MS(+) m/z [M+2H]²⁺: 1175.1.

### Preparation of Example 2029

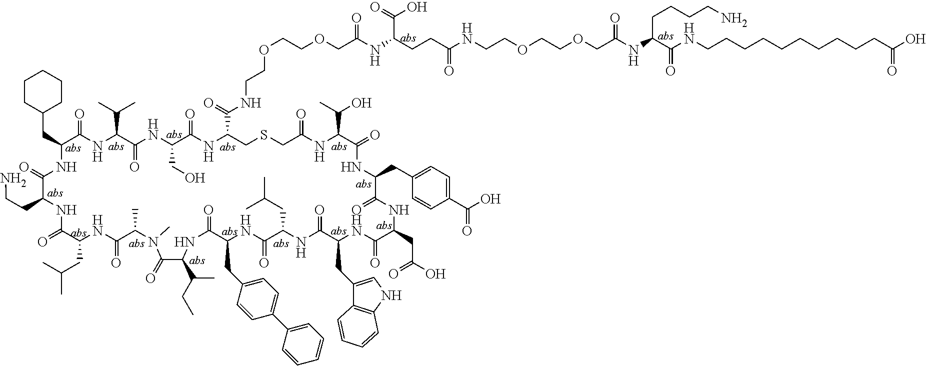

Example 2029 was prepared on a 25 μmol scale. The yield of the product was 40.9 mg, and its estimated purity by LCMS analysis was 100%. Analysis condition A: Retention time=1.71 min; ESI-MS(+) m/z [M+2H]²⁺: 1294.9.

### Preparation of Example 2030

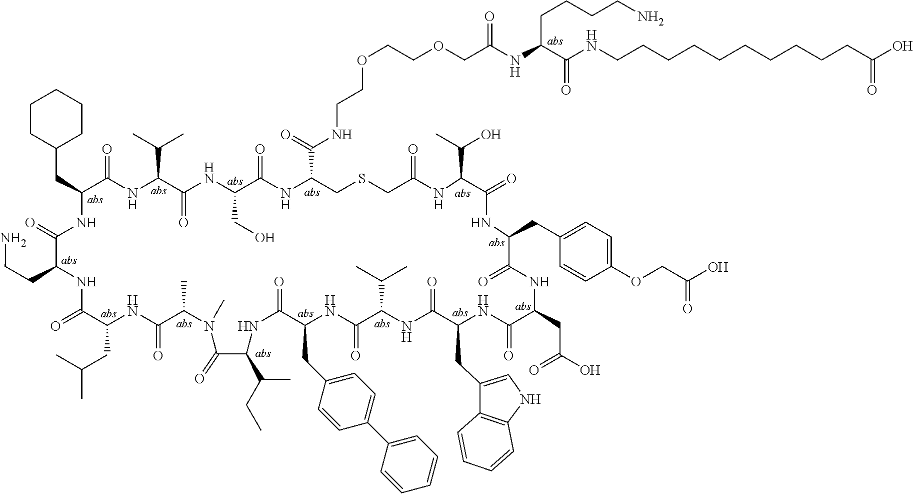

Example 2030 was prepared on a 25 μmol scale. The yield of the product was 30.8 mg, and its estimated purity by LCMS analysis was 97%. Analysis condition B: Retention time=1.86 min; ESI-MS(+) m/z [M+2H]²⁺: 1158.1.

### Preparation of Example 2031

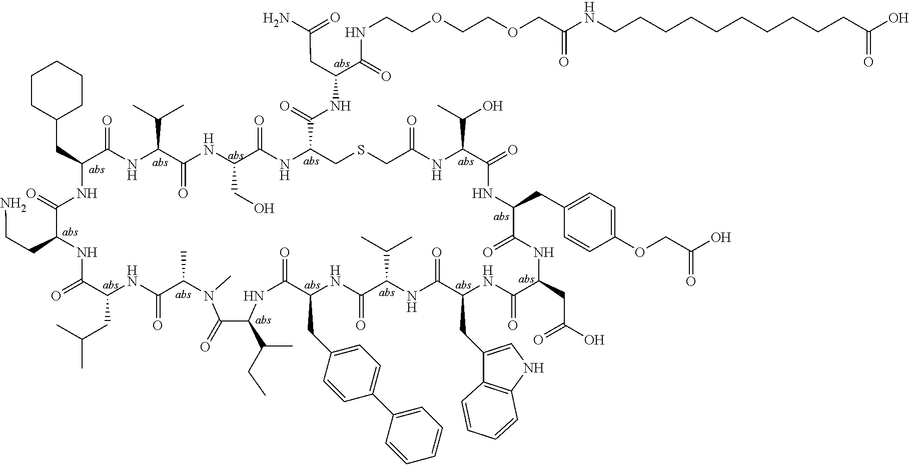

Example 2031 was prepared on a 25 μmol scale. The yield of the product was 28.3 mg, and its estimated purity by LCMS analysis was 98.1%. Analysis condition B: Retention time=1.96 min; ESI-MS(+) m/z [M+2H]²⁺: 1151.1.

### Preparation of Example 2032

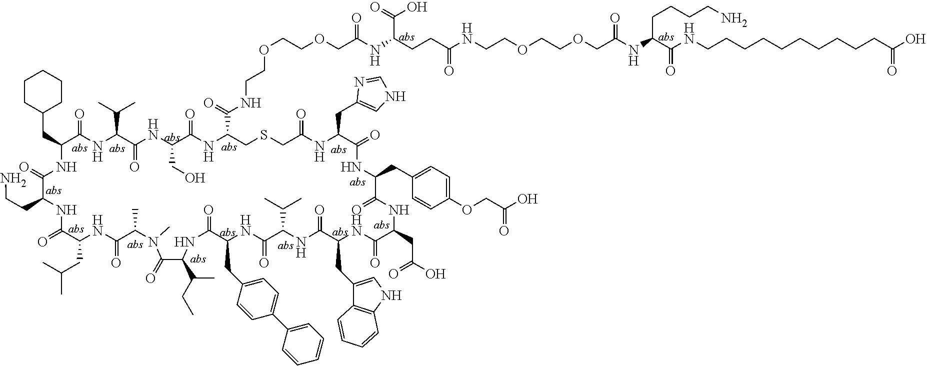

Example 2032 was prepared on a 25 μmol scale. The yield of the product was 31.8 mg, and its estimated purity by LCMS analysis was 100%. Analysis condition A: Retention time=1.73 min; ESI-MS(+) m/z [M+2H]²⁺: 1313.1.

### Preparation of Example 2033

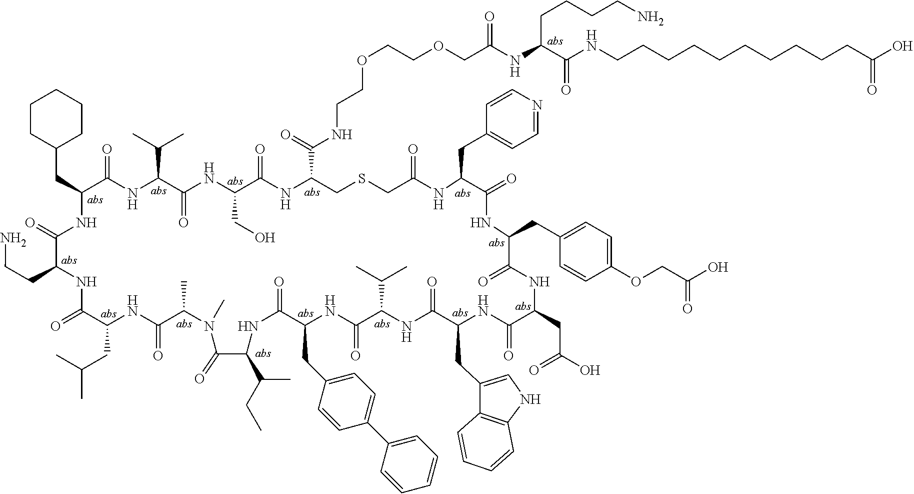

Example 2033 was prepared on a 25 μmol scale. The yield of the product was 22.8 mg, and its estimated purity by LCMS analysis was 100%. Analysis condition A: Retention time=1.79 min; ESI-MS(+) m/z [M+2H]²⁺: 1182.

### Preparation of Example 2034

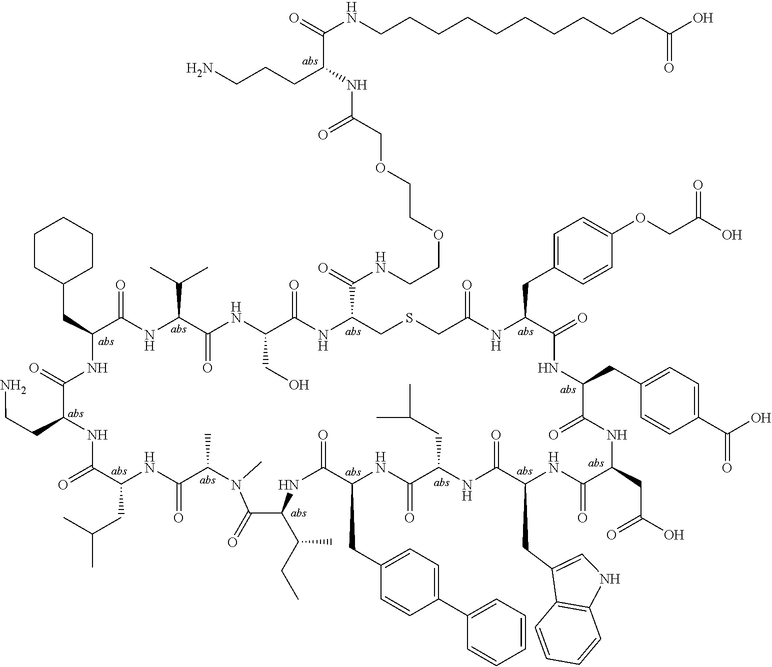

Example 2034 was prepared on a 50 μmol scale. The yield of the product was 3.3 mg, and its estimated purity by LCMS analysis was 98.4%. Analysis condition A: Retention time=1.72 min; ESI-MS(+) m/z [M+2H]²⁺: 1202.9.

### Preparation of Example 2035

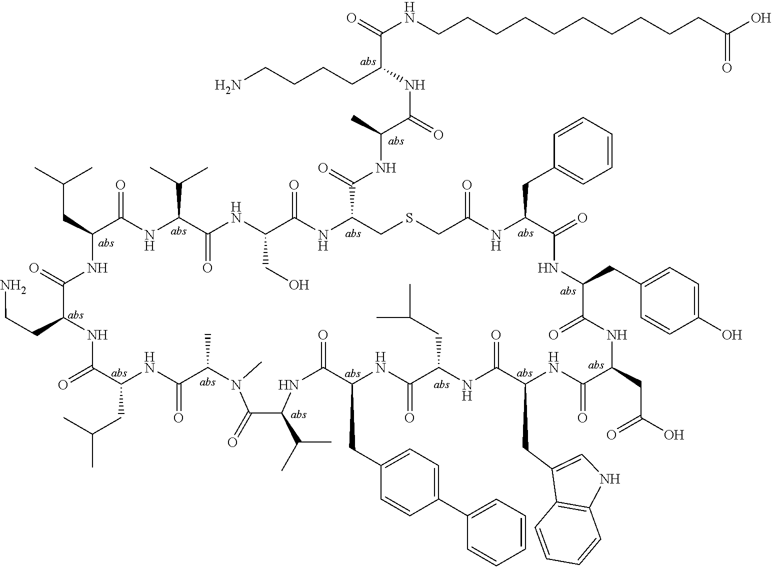

Example 2035 was prepared on a 50 μmol scale. The yield of the product was 13 mg, and its estimated purity by LCMS analysis was 96.4%. Analysis condition B: Retention time=1.85 min; ESI-MS(+) m/z [M+2H]²⁺: 1094.7.

### Preparation of Example 2036

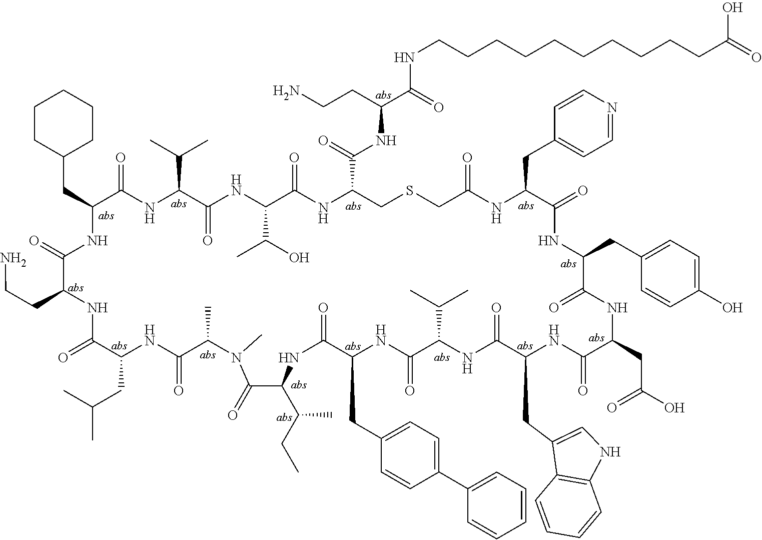

Example 2036 was prepared on a 50 μmol scale. The yield of the product was 38 mg, and its estimated purity by LCMS analysis was 98.2%. Analysis condition B: Retention time=1.72 min; ESI-MS(+) m/z [M+2H]²⁺: 1073.

### Preparation of Example 2037

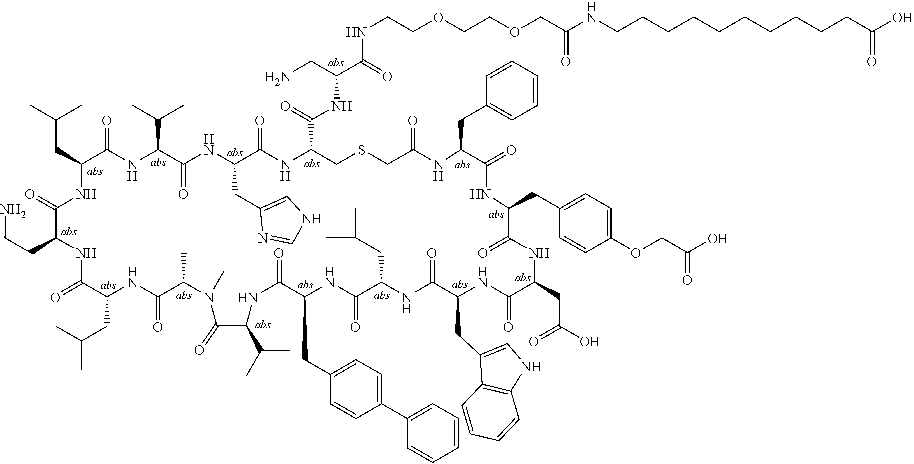

Example 2037 was prepared on a 50 μmol scale. The yield of the product was 62.7 mg, and its estimated purity by LCMS analysis was 95.8%. Analysis condition B: Retention time=1.74 min; ESI-MS(+) m/z [M+2H]²⁺: 1165.

### Preparation of Example 2038

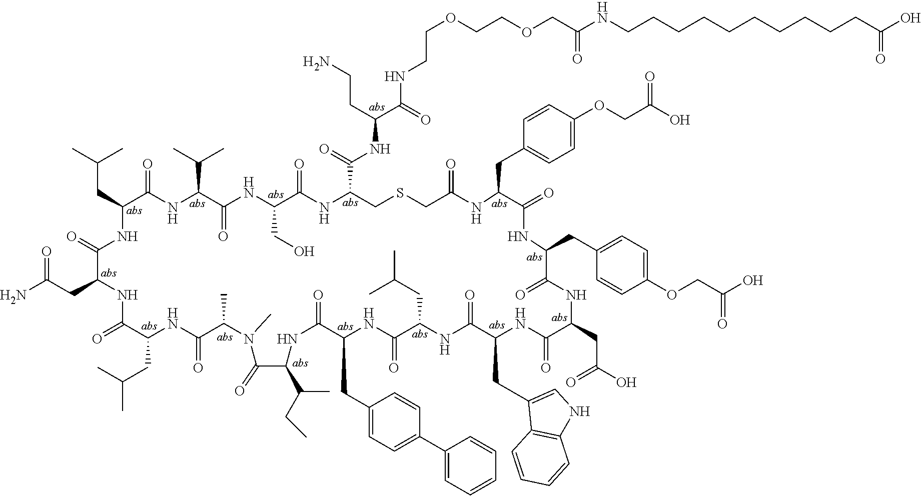

Example 2038 was prepared on a 50 μmol scale. The yield of the product was 18.5 mg, and its estimated purity by LCMS analysis was 100%. Analysis condition A: Retention time=1.52 min; ESI-MS(+) m/z [M+2H]²⁺: 1198.2.

### Preparation of Example 2039

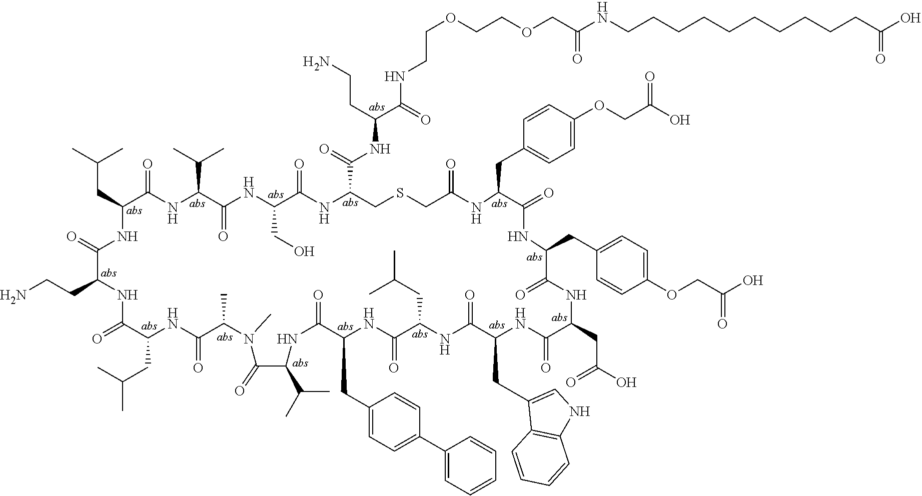

Example 2039 was prepared on a 50 μmol scale. The yield of the product was 18.2 mg, and its estimated purity by LCMS analysis was 97.2%. Analysis condition B Retention time=1.96 min; ESI-MS(+) m/z [M+2H]²⁺: 1184.0.

### Preparation of Example 2040

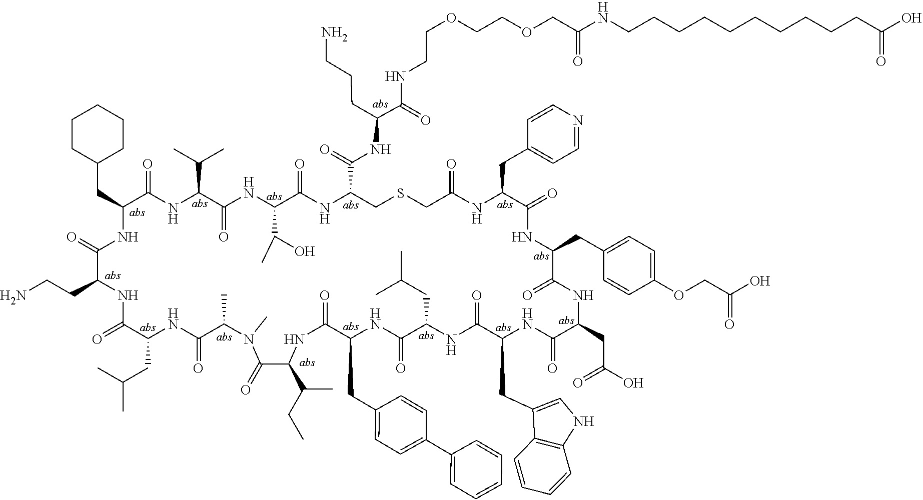

Example 2040 was prepared on a 50 μmol scale. The yield of the product was 33.8 mg, and its estimated purity by LCMS analysis was 98.3%. Analysis condition B: Retention time=1.79 min; ESI-MS(+) m/z [M+2H]²⁺: 1189.1.

### Preparation of Example 2041

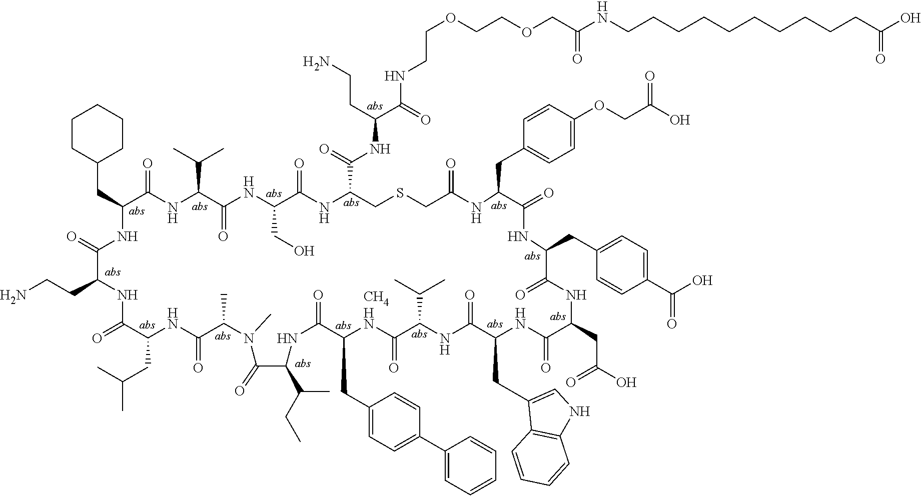

Example 2041 was prepared on a 50 μmol scale. The yield of the product was 8.4 mg, and its estimated purity by LCMS analysis was 100%. Analysis condition A: Retention time=1.54 min; ESI-MS(+) m/z [M+2H]²⁺: 1197.5.

### Preparation of Example 2042

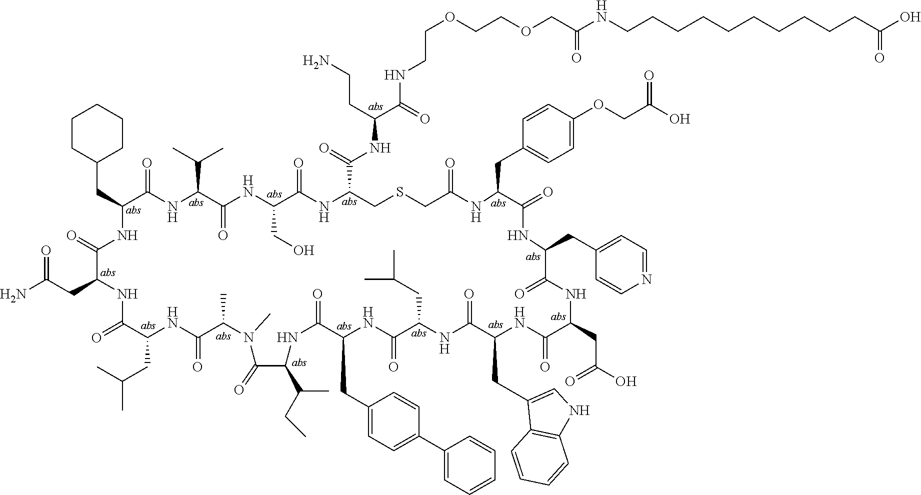 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2042 was prepared on a 50 μmol scale. The yield of the product was 6.3 mg, and its estimated purity by LCMS analysis was 100%. Analysis condition A: Retention time=1.68 min; ESI-MS(+) m/z [M+2H]²⁺: 1162.1.

### Preparation of Example 2043

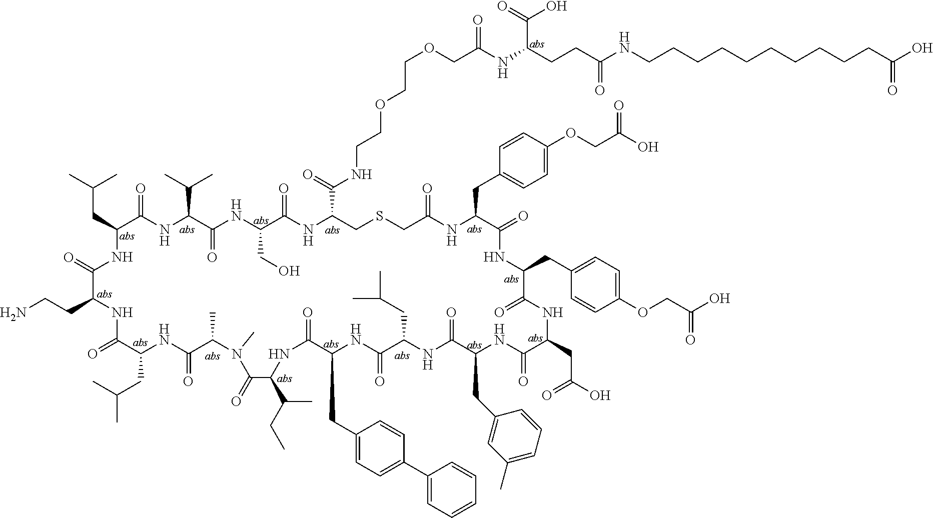 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2043 was prepared on a 50 μmol scale. The yield of the product was 14.3 mg, and its estimated purity by LCMS analysis was 99.3%. Analysis condition B: Retention time=1.95 min; ESI-MS(+) m/z [M+2H]²⁺: 1193.2.

### Preparation of Example 2044

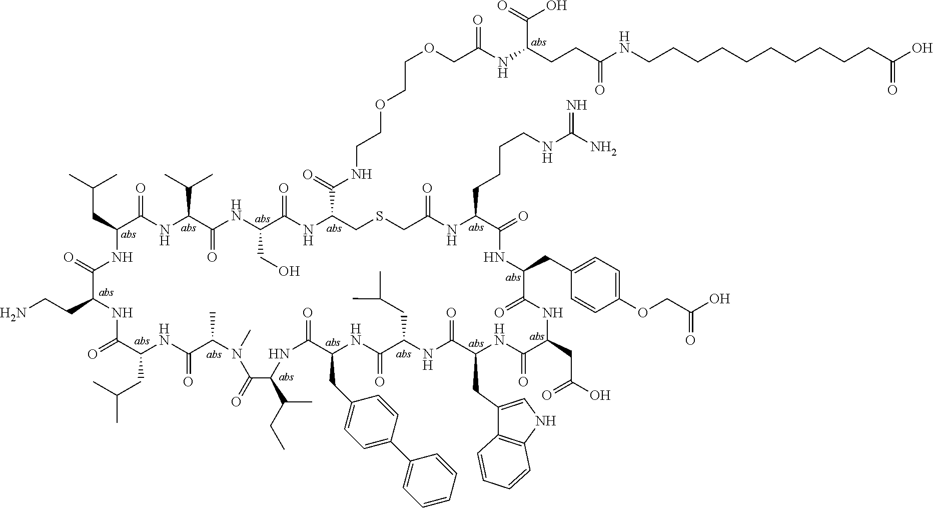 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2044 was prepared on a 50 μmol scale. The yield of the product was 30.6 mg, and its estimated purity by LCMS analysis was 96.1%. Analysis condition B: Retention time=1.84 min; ESI-MS(+) m/z [M+2H]²⁺: 1180.2.

### Preparation of Example 2045

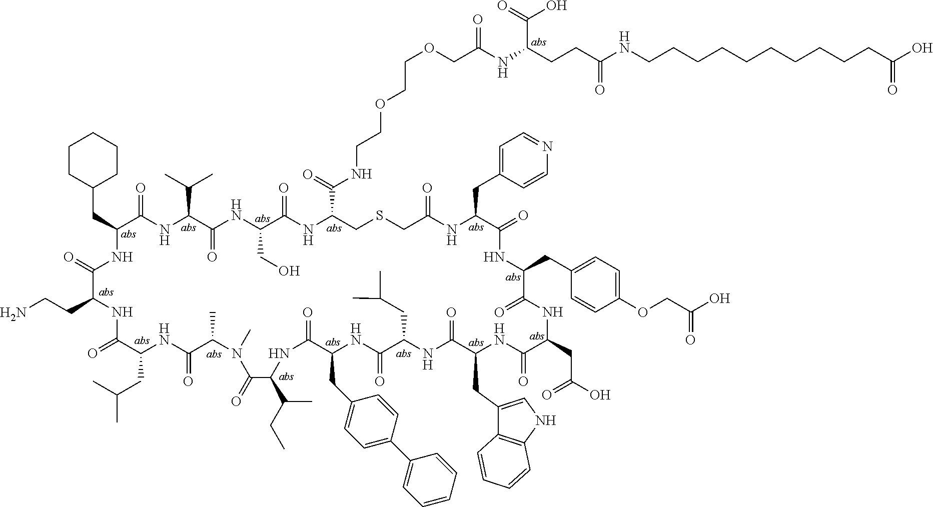 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)N[C@@H](CCC(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2045 was prepared on a 50 μmol scale. The yield of the product was 60.2 mg, and its estimated purity by LCMS analysis was 95.6%. Analysis condition A: Retention time=1.7 min; ESI-MS(+) m/z [M+2H]²⁺: 1188.8.

### Preparation of Example 2046

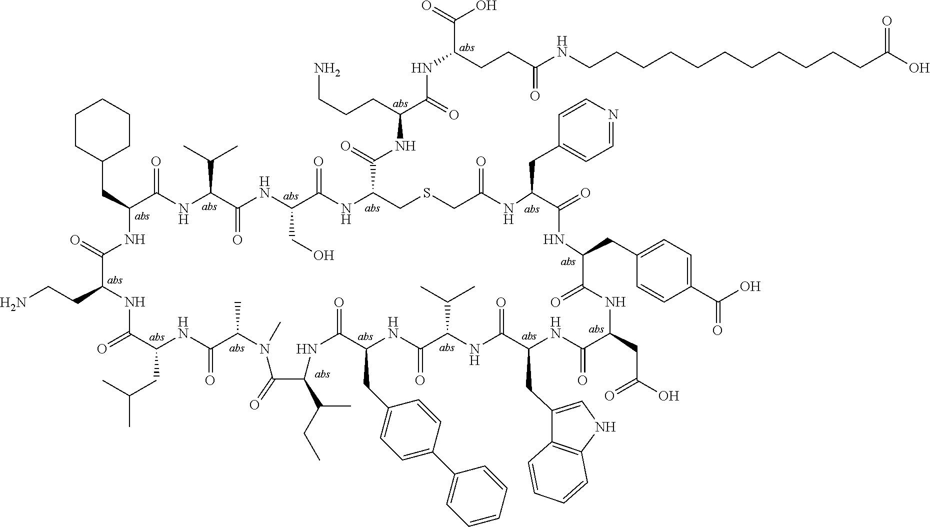 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCCN)C(=O)N[C@@H](CCC(=O)NCCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2046 was prepared on a 25 μmol scale. The yield of the product was 6 mg, and its estimated purity by LCMS analysis was 92.6%. Analysis condition B: Retention time=1.7 min; ESI-MS(+) m/z [M+2H]²⁺: 1173.9.

### Preparation of Example 2047

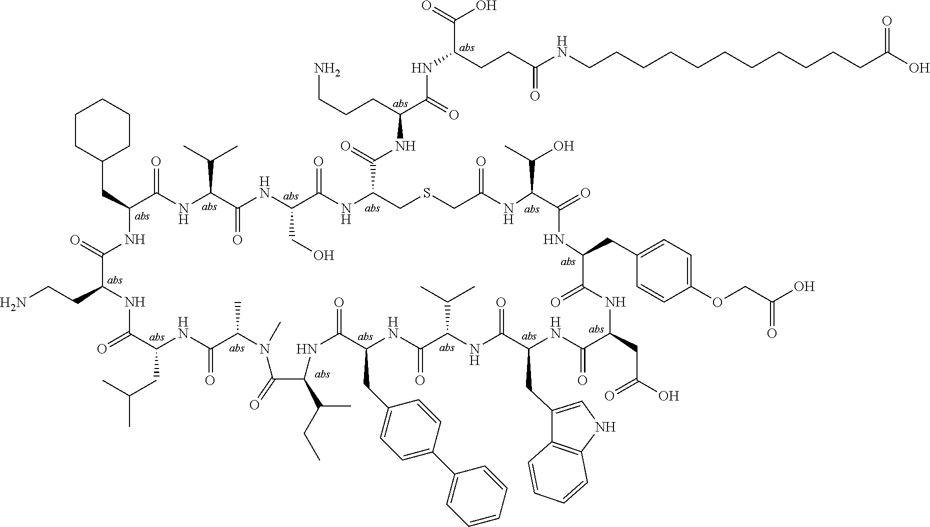 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CCC(=O)NCCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2047 was prepared on a 25 μmol scale. The yield of the product was 6.7 mg, and its estimated purity by LCMS analysis was 96.9%. Analysis condition A: Retention time=1.58 min; ESI-MS(+) m/z [M+2H]²⁺: 1149.9.

### Preparation of Example 2048

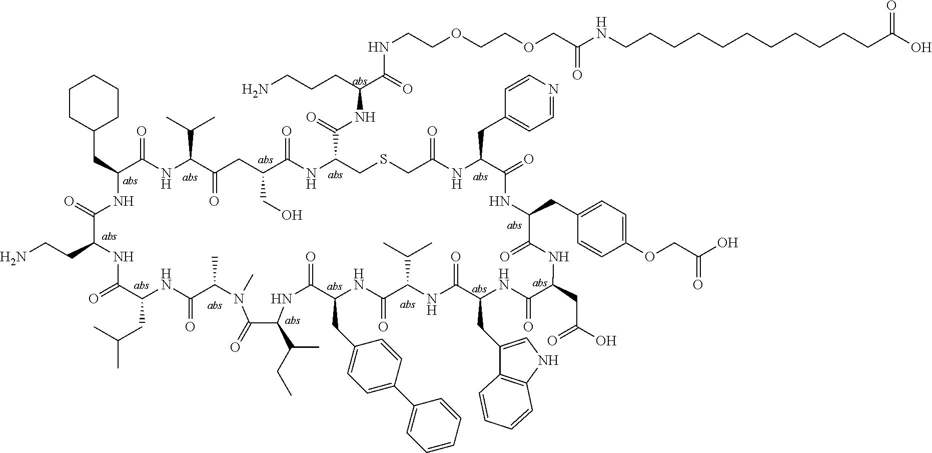 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2048 was prepared on a 25 μmol scale. The yield of the product was 5.8 mg, and its estimated purity by LCMS analysis was 96%. Analysis condition B: Retention time=1.73 min; ESI-MS(+) m/z [M+2H]²⁺: 1181.

### Preparation of Example 2049

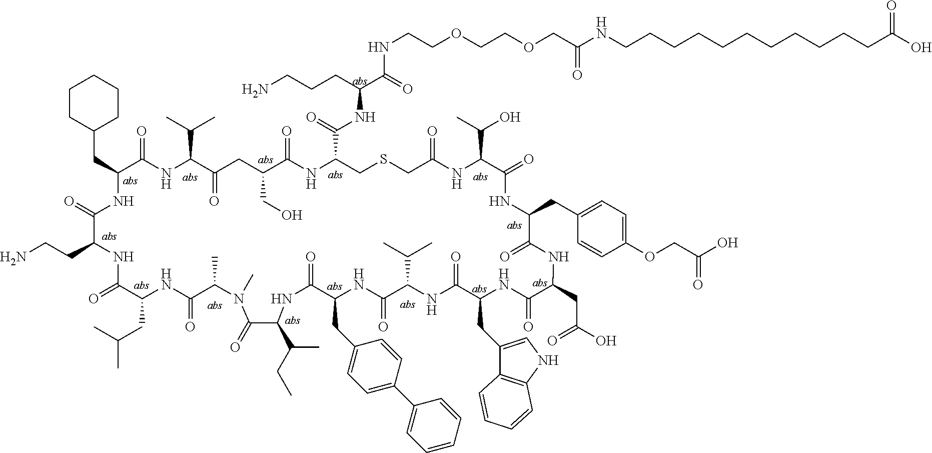 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)NCCOCCOCC(=O)NCCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2049 was prepared on a 25 μmol scale. The yield of the product was 7.7 mg, and its estimated purity by LCMS analysis was 99%. Analysis condition B: Retention time=1.78 min; ESI-MS(+) m/z [M+2H]²⁺: 1158.4.

### Preparation of Example 2050

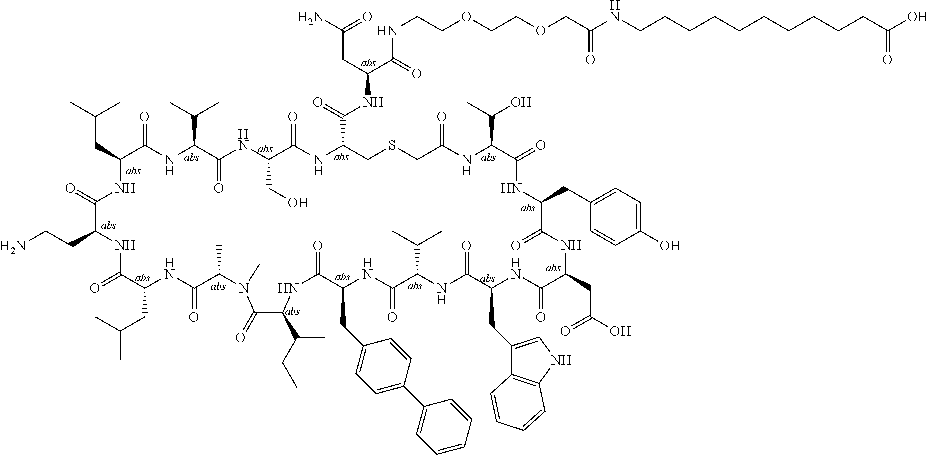 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](C(C)O)NC(=O)CSC[C@@H](C(=O)N[C@@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2050 was prepared on a 25 μmol scale. The yield of the product was 21.9 mg, and its estimated purity by LCMS analysis was 95.9%. Analysis condition B: Retention time=1.71 min; ESI-MS(+) m/z [M+2H]²⁺: 1102.

### Preparation of Example 2051

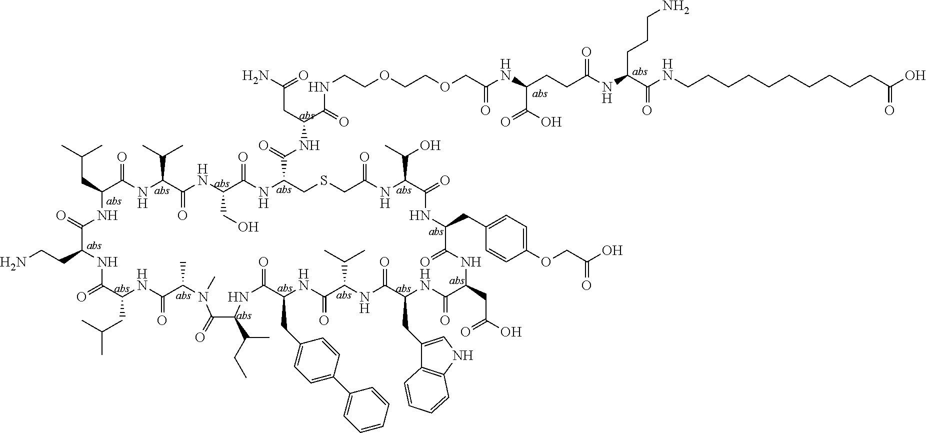 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N[C@@H](CCC(=O)N[C@@H](CCCN)C(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2051 was prepared on a 25 μmol scale. The yield of the product was 10.9 mg, and its estimated purity by LCMS analysis was 90.9%. Analysis condition B: Retention time=1.66 min; ESI-MS(+) m/z [M+2H]²⁺: 1253.1.

### Preparation of Example 2052

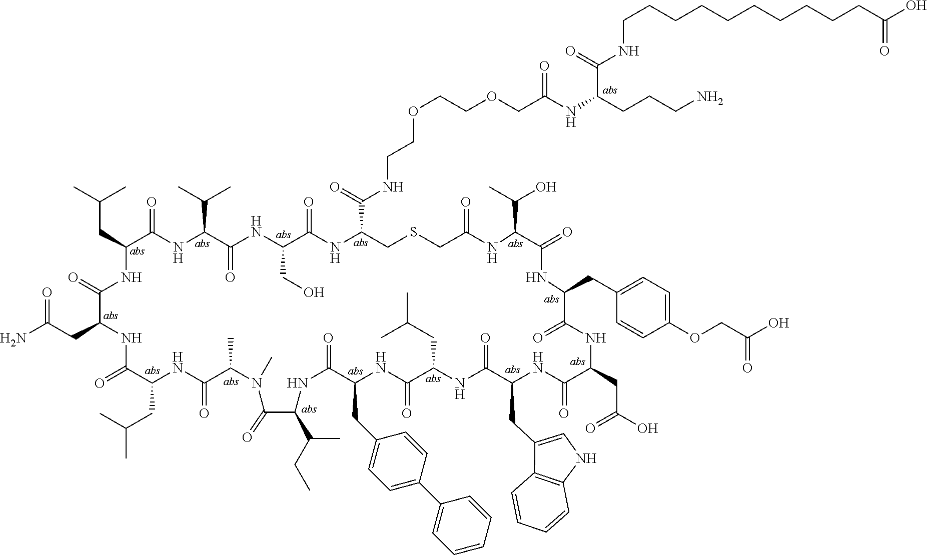 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C1=O]

Example 2052 was prepared on a 25 μmol scale. The yield of the product was 9.5 mg, and its estimated purity by LCMS analysis was 96.5%. Analysis condition A: Retention time=1.7 min; ESI-MS(+) m/z [M+2H]²⁺: 1144.9.

### Preparation of Example 2053

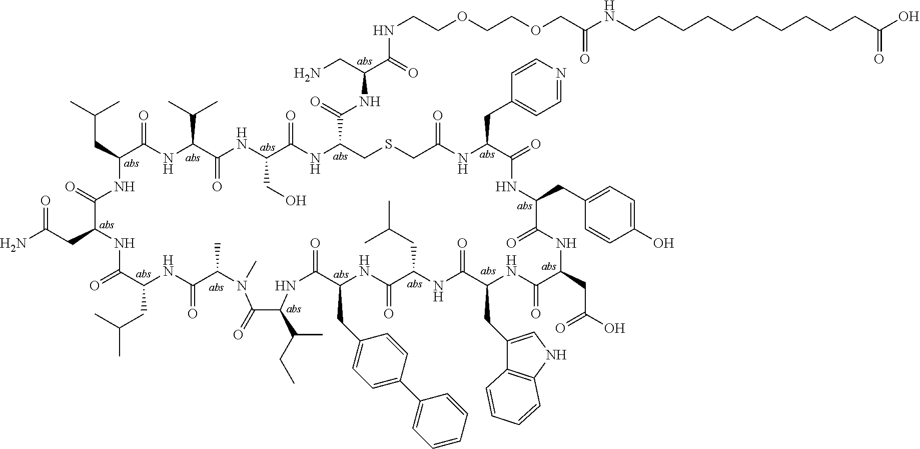 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CN)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C1=O]

Example 2053 was prepared on a 25 μmol scale. The yield of the product was 1.8 mg, and its estimated purity by LCMS analysis was 95.6%. Analysis condition B: Retention time=2.02 min; ESI-MS(+) m/z [M+2H]²⁺: 1125.2.

### Preparation of Example 2054

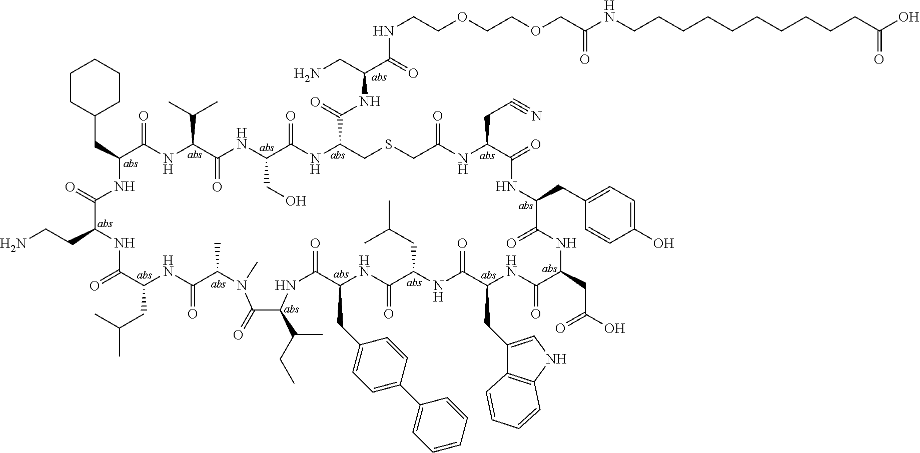 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC#N)NC(=O)CSC[C@@H](C(=O)N[C@@H](CN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2054 was prepared on a 25 μmol scale. The yield of the product was 7.7 mg, and its estimated purity by LCMS analysis was 96%. Analysis condition B: Retention time=1.96 min; ESI-MS(+) m/z [M+2H]²⁺: 1112.3.

### Preparation of Example 2055

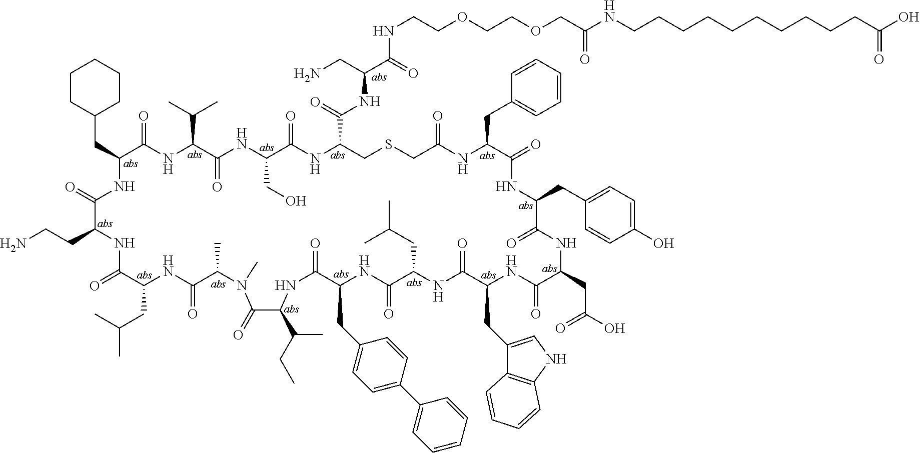 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2055 was prepared on a 25 μmol scale. The yield of the product was 2.4 mg, and its estimated purity by LCMS analysis was 96.4%. Analysis condition B: Retention time=1.99 min; ESI-MS(+) m/z [M+2H]²⁺: 1138.2.

### Preparation of Example 2056

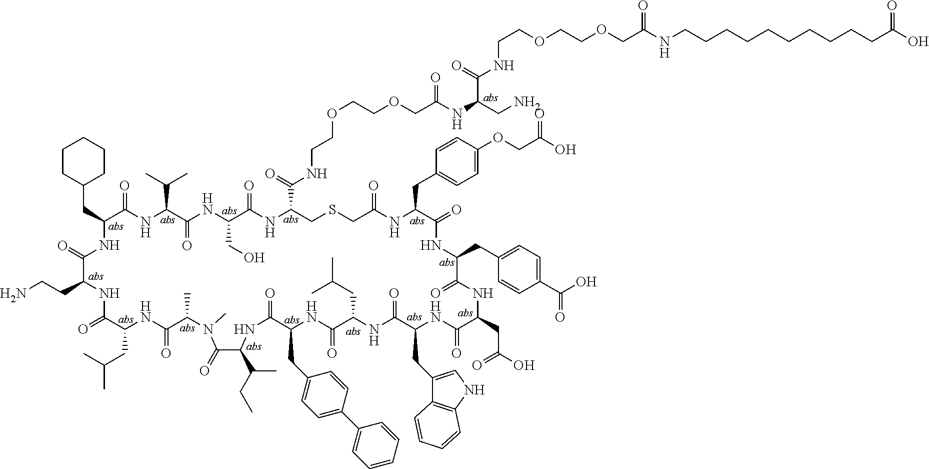 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@H](CN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2056 was prepared on a 50 μmol scale. The yield of the product was 33.7 mg, and its estimated purity by LCMS analysis was 95.1%. Analysis condition B: Retention time=1.93 min; ESI-MS(+) m/z [M+2H]²⁺: 1261.2.

### Preparation of Example 2057

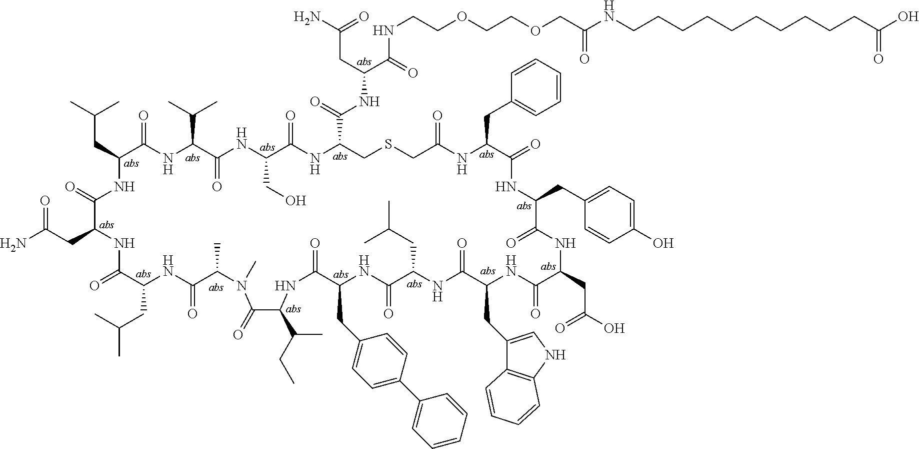 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C1=O]

Example 2057 was prepared on a 25 μmol scale. The yield of the product was 11.5 mg, and its estimated purity by LCMS analysis was 98.2%. Analysis condition B: Retention time=1.93 min; ESI-MS(+) m/z [M+2H]²⁺: 1132.

### Preparation of Example 2058

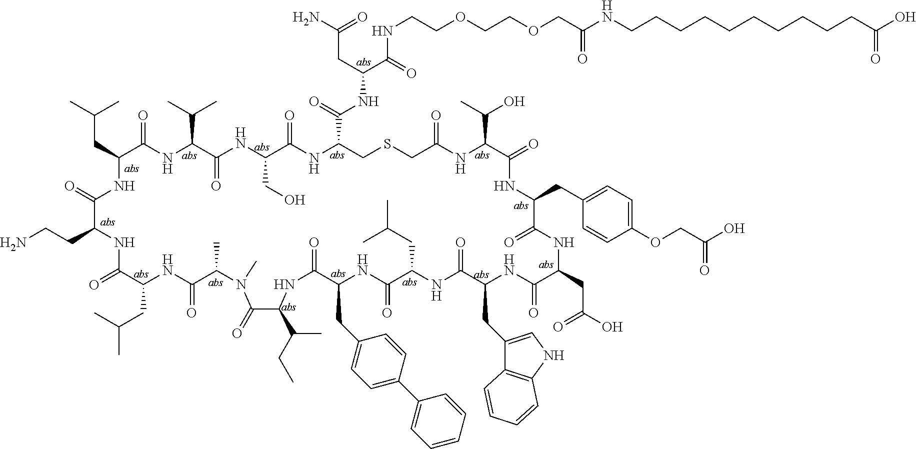 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2058 was prepared on a 25 μmol scale. The yield of the product was 9.7 mg, and its estimated purity by LCMS analysis was 97.8%. Analysis condition A: Retention time=1.66 min; ESI-MS(+) m/z [M+2H]²⁺: 1138.1.

### Preparation of Example 2059

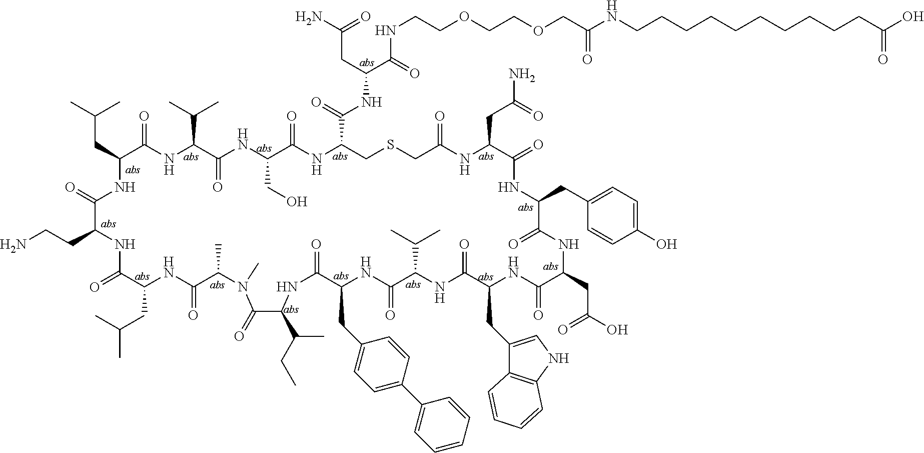 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2059 was prepared on a 25 μmol scale. The yield of the product was 7.8 mg, and its estimated purity by LCMS analysis was 98.7%. Analysis condition A: Retention time=1.61 min; ESI-MS(+) m/z [M+2H]²⁺: 1108.2.

### Preparation of Example 2060

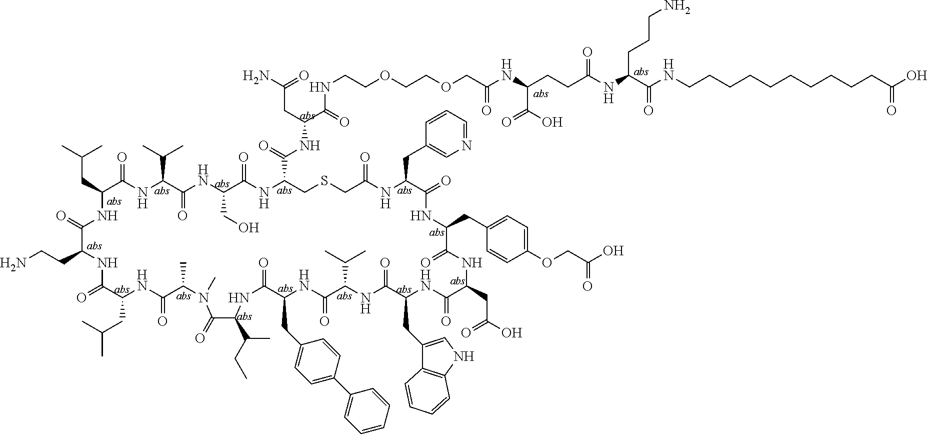 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cccnc2)NC(=O)CSC[C@@H](C(=O)N[C@H](CC(N)=O)C(=O)NCCOCCOCC(=O)N[C@@H](CCC(=O)N[C@@H](CCCN)C(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2060 was prepared on a 25 μmol scale. The yield of the product was 16.2 mg, and its estimated purity by LCMS analysis was 88.6%. Analysis condition A: Retention time=1.5 min; ESI-MS(+) m/z [M+2H]²⁺: 1276.

### Preparation of Example 2061

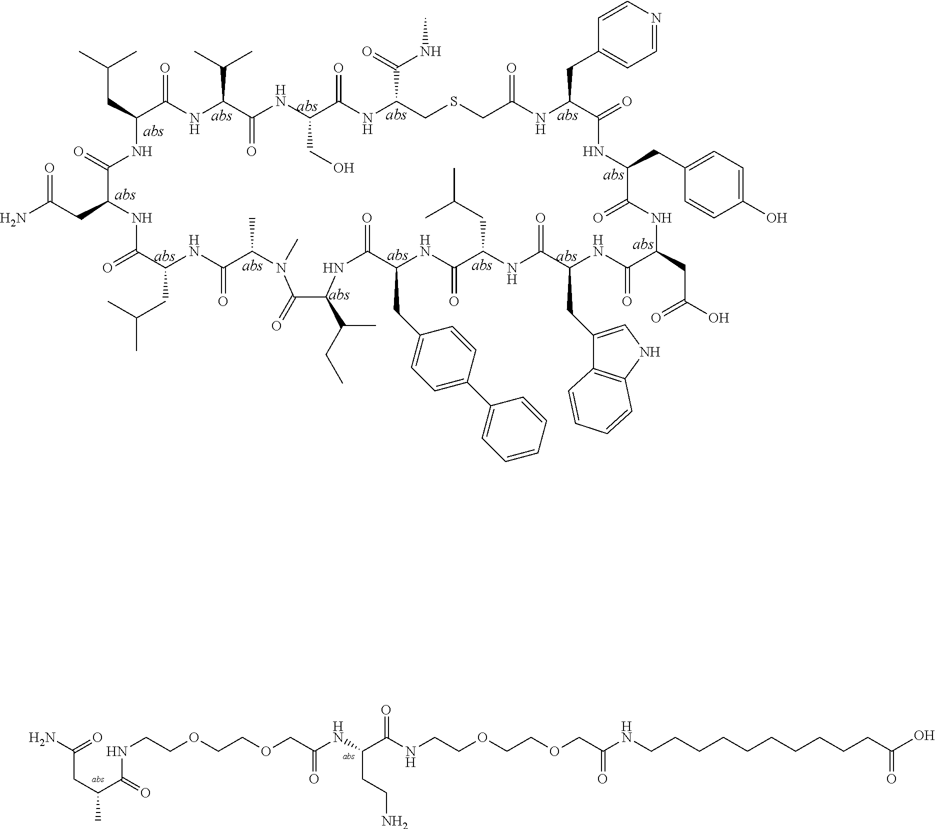

Example 2061 was prepared on a 25 μmol scale. The yield of the product was 8.1 mg, and its estimated purity by LCMS analysis was 100%. Analysis condition A: Retention time=1.72 min; ESI-MS(+) m/z [M+2H]²⁺: 1262.2.

### Preparation of Example 2062

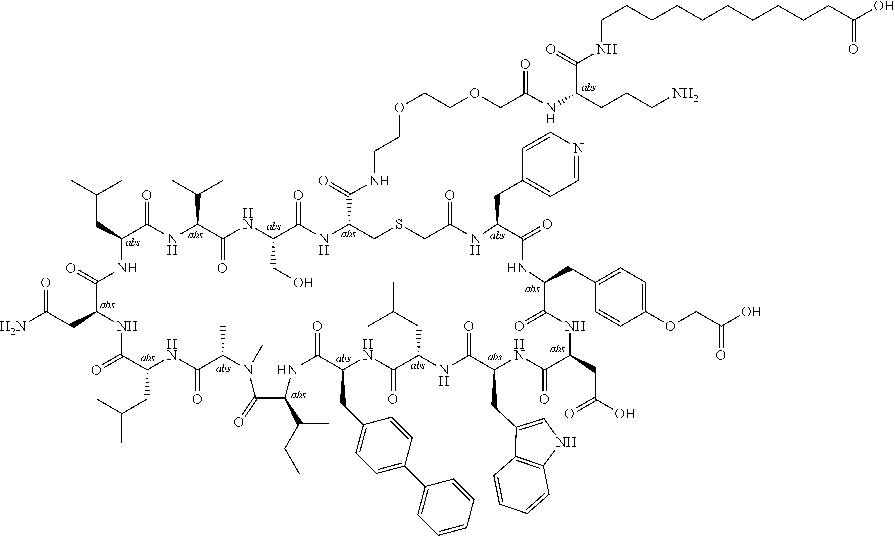 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](C)N(C)C1=O]

Example 2062 was prepared on a 25 μmol scale. The yield of the product was 7.2 mg, and its estimated purity by LCMS analysis was 97.5%. Analysis condition A: Retention time=1.55 min; ESI-MS(+) m/z [M+3H]³⁺: 779.1.

### Preparation of Example 2063

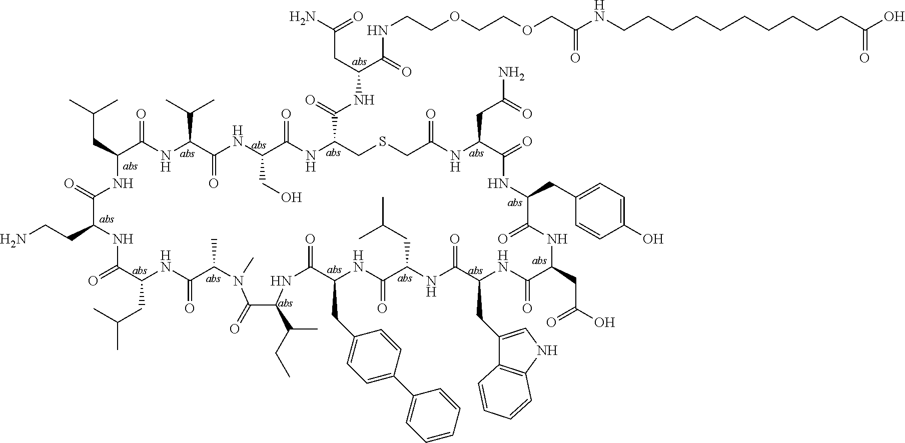 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2063 was prepared on a 25 μmol scale. The yield of the product was 8.8 mg, and its estimated purity by LCMS analysis was 100%. Analysis condition A: Retention time=1.59 min; ESI-MS(+) m/z [M+2H]²⁺: 1115.1.

### Preparation of Example 2064

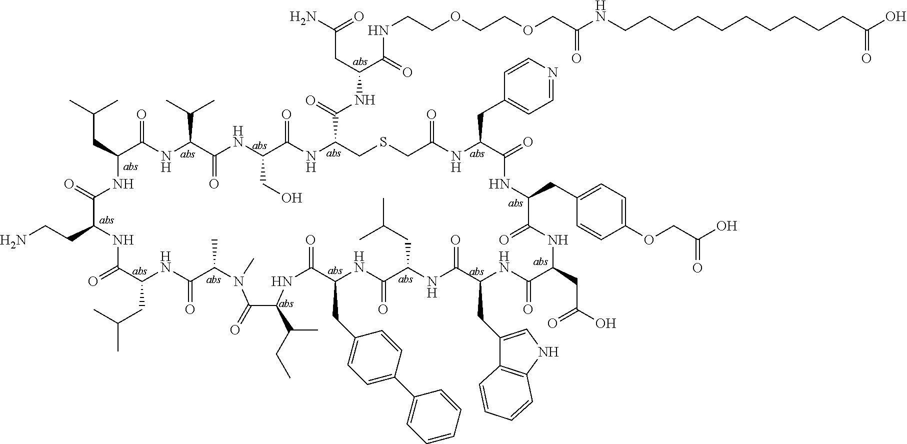 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2064 was prepared on a 25 μmol scale. The yield of the product was 16.7 mg, and its estimated purity by LCMS analysis was 97.5%. Analysis condition A: Retention time=1.64 min; ESI-MS(+) m/z [M+2H]²⁺: 1161.2.

### Preparation of Example 2065

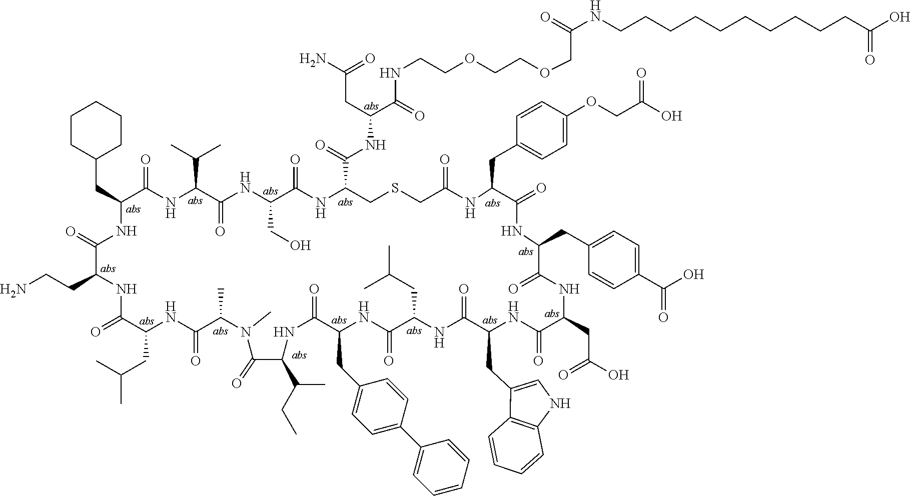 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2065 was prepared on a 50 μmol scale. The yield of the product was 29.9 mg, and its estimated purity by LCMS analysis was 99.5%. Analysis condition B: Retention time=1.95 min; ESI-MS(+) m/z [M+2H]²⁺: 1202.9.

### Preparation of Example 2066

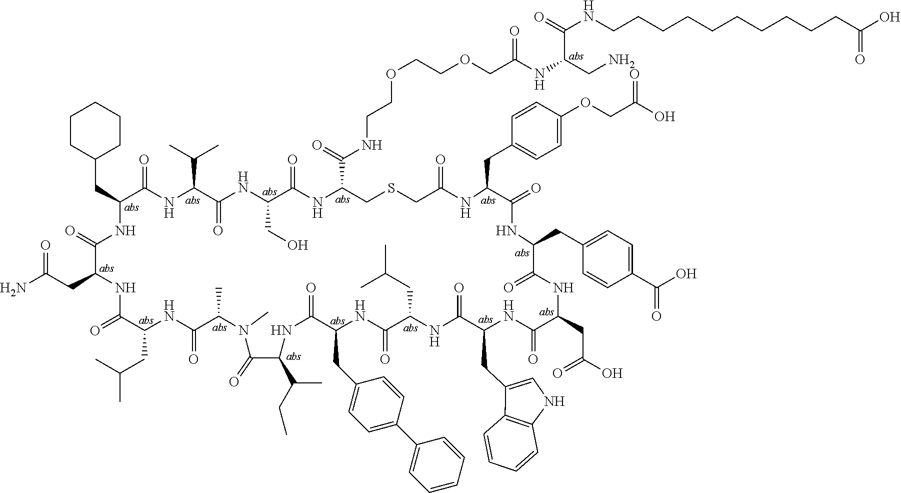 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CN)C(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2066 was prepared on a 50 μmol scale. The yield of the product was 47.8 mg, and its estimated purity by LCMS analysis was 96.4%. Analysis condition B: Retention time=2.18 min; ESI-MS(+) m/z [M+2H]²⁺: 1196.1.

### Preparation of Example 2067

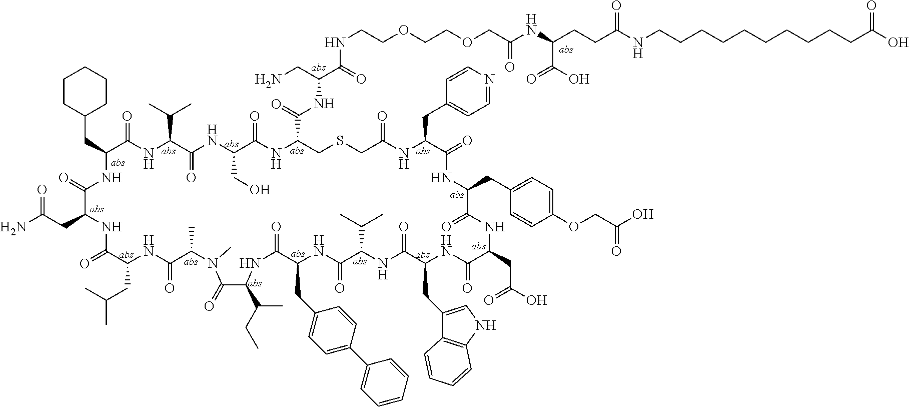 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@H](CN)C(=O)NCCOCCOCC(=O)N[C@@H](CCC(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2067 was prepared on a 50 μmol scale. The yield of the product was 13.1 mg, and its estimated purity by LCMS analysis was 90.9%. Analysis condition B: Retention time=2 min; ESI-MS(+) m/z [M+2H]²⁺: 1231.9.

### Preparation of Example 2068

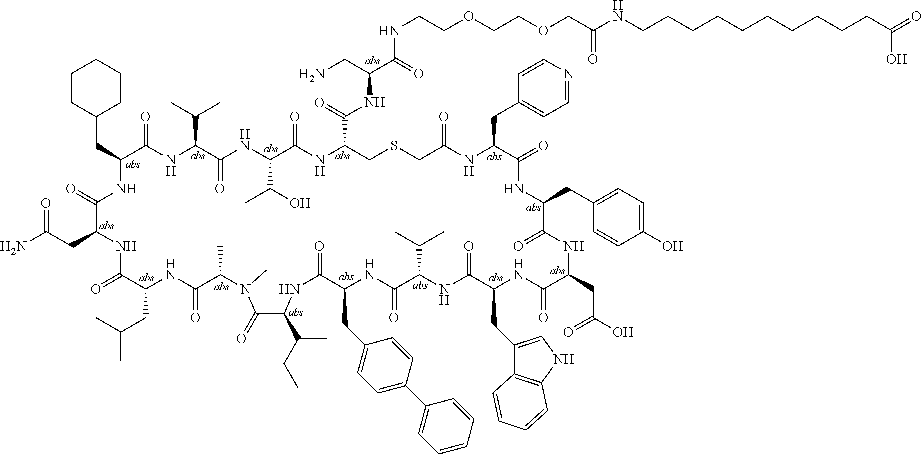 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2068 was prepared on a 50 μmol scale. The yield of the product was 11.8 mg, and its estimated purity by LCMS analysis was 98.9%. Analysis condition A: Retention time=1.93 min; ESI-MS(+) m/z [M+2H]²⁺: 1145.3.

### Preparation of Example 2069

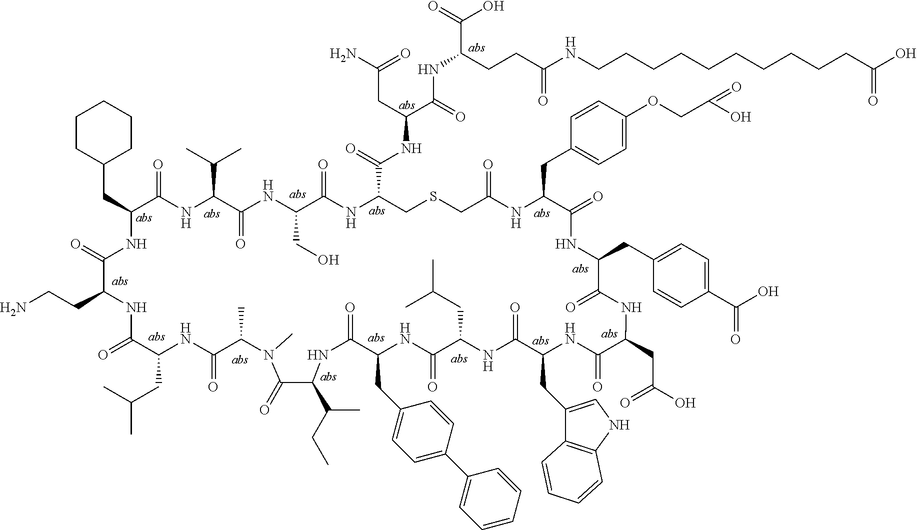 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)N[C@@H](CCC(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2069 was prepared on a 50 μmol scale. The yield of the product was 18.7 mg, and its estimated purity by LCMS analysis was 92%. Analysis condition B: Retention time=1.99 min; ESI-MS(+) m/z [M+2H]²⁺: 1195.1.

### Preparation of Example 2070

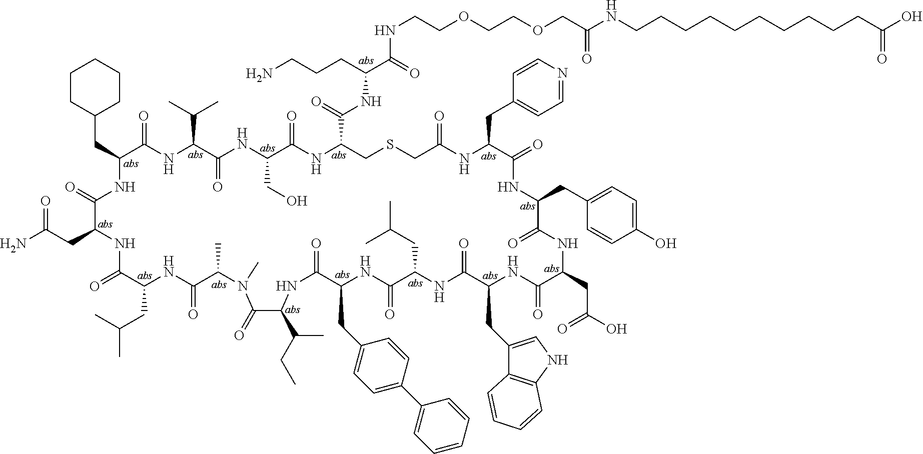 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2070 was prepared on a 50 μmol scale. The yield of the product was 26.1 mg, and its estimated purity by LCMS analysis was 98.9%. Analysis condition B: Retention time=2.09 min; ESI-MS(+) m/z [M+2H]²⁺: 1159.1.

### Preparation of Example 2071

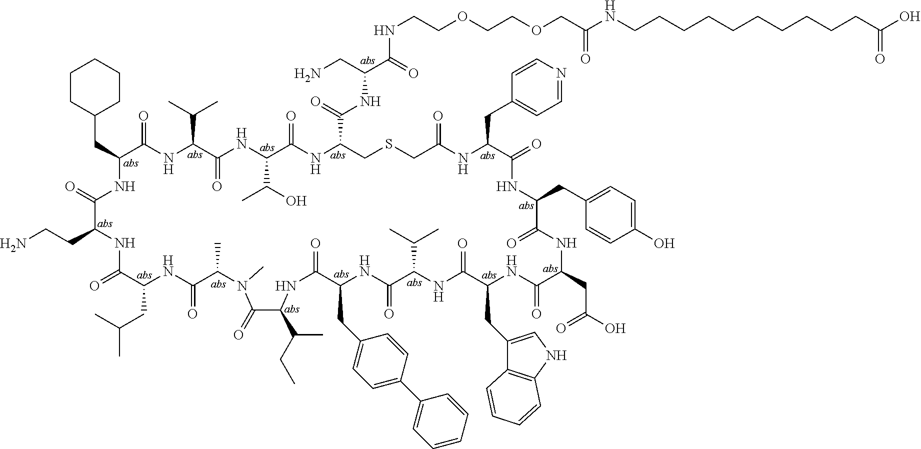 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2071 was prepared on a 50 μmol scale. The yield of the product was 20.6 mg, and its estimated purity by LCMS analysis was 98.9%. Analysis condition A: Retention time=1.64 min; ESI-MS(+) m/z [M+2H]²⁺: 1138.8.

### Preparation of Example 2072

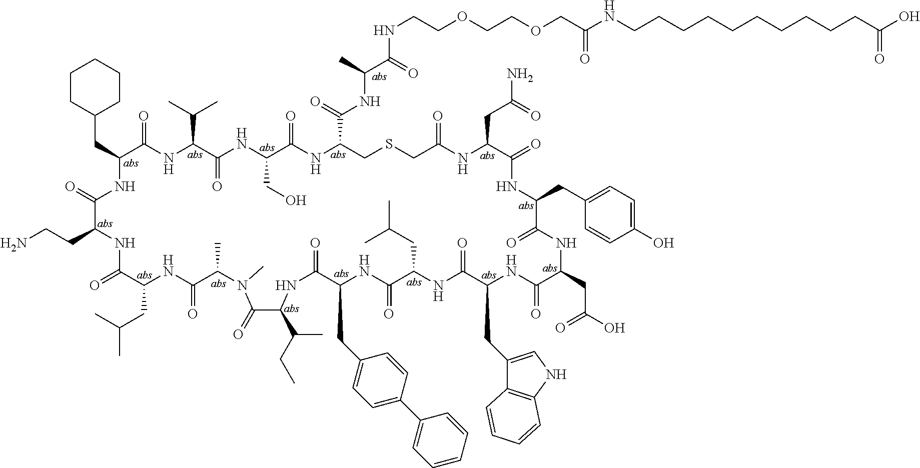 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2072 was prepared on a 50 μmol scale. The yield of the product was 12.6 mg, and its estimated purity by LCMS analysis was 98.9%. Analysis condition A: Retention time=1.9 min; ESI-MS(+) m/z [M+2H]²⁺: 1113.8.

### Preparation of Example 2073

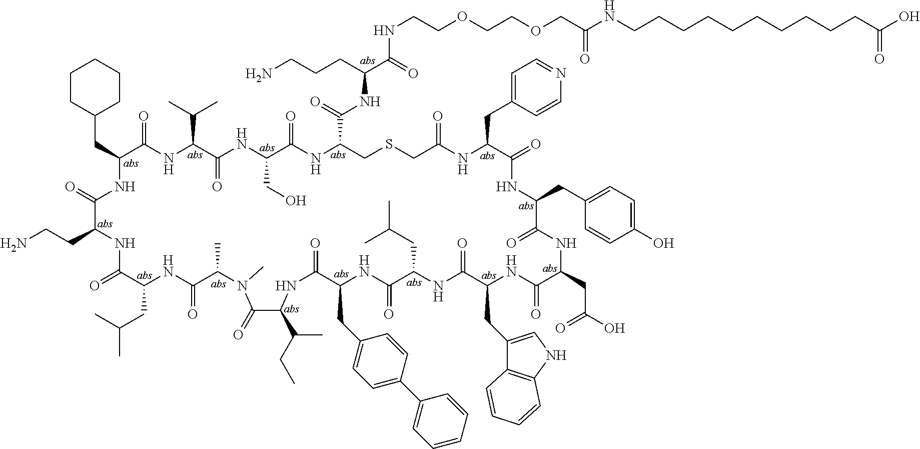 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2073 was prepared on a 50 μmol scale. The yield of the product was 22.3 mg, and its estimated purity by LCMS analysis was 100%. Analysis condition A: Retention time=1.77 min; ESI-MS(+) m/z [M+2H]²⁺: 1153.1.

### Preparation of Example 2074

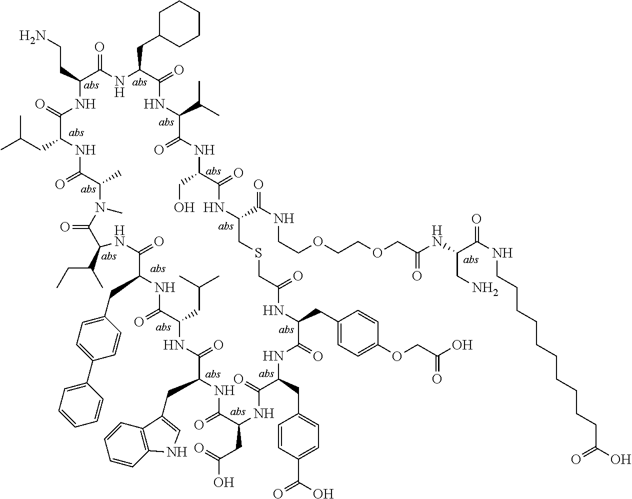 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2074 was prepared on a 50 μmol scale. The yield of the product was 25.9 mg, and its estimated purity by LCMS analysis was 100%. Analysis condition A: Retention time=1.6 min; ESI-MS(+) m/z [M+2H]²⁺: 1189.3.

### Preparation of Example 2075

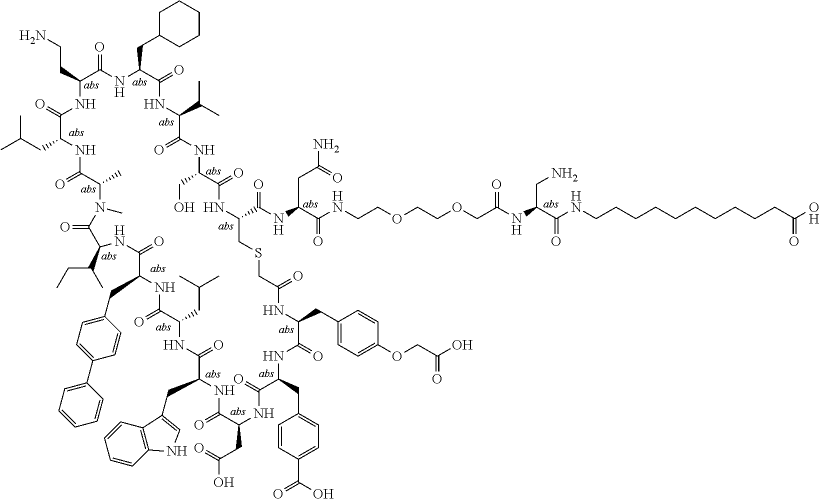 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCC(=O)N[C@@H](CN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2075 was prepared on a 50 μmol scale. The yield of the product was 17.7 mg, and its estimated purity by LCMS analysis was 97.9%. Analysis condition B: Retention time=1.88 min; ESI-MS(+) m/z [M+2H]²⁺: 1246.1.

### Preparation of Example 2076

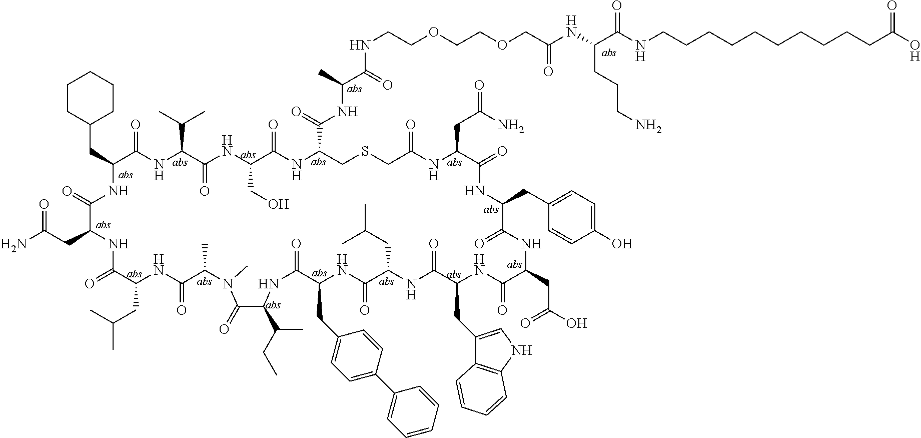 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C1=O]

Example 2076 was prepared on a 50 μmol scale. The yield of the product was 65.3 mg, and its estimated purity by LCMS analysis was 90.2%. Analysis condition B: Retention time=2.14 min; ESI-MS(+) m/z [M+2H]²⁺: 1178.3.

### Preparation of Example 2077

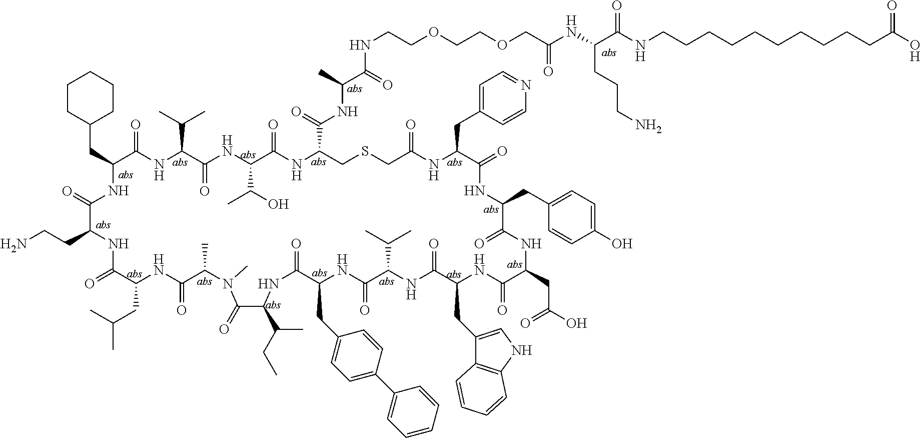 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2077 was prepared on a 50 μmol scale. The yield of the product was 43 mg, and its estimated purity by LCMS analysis was 100%. Analysis condition A: Retention time=1.75 min; ESI-MS(+) m/z [M+2H]²⁺: 1188.1.

### Preparation of Example 2078

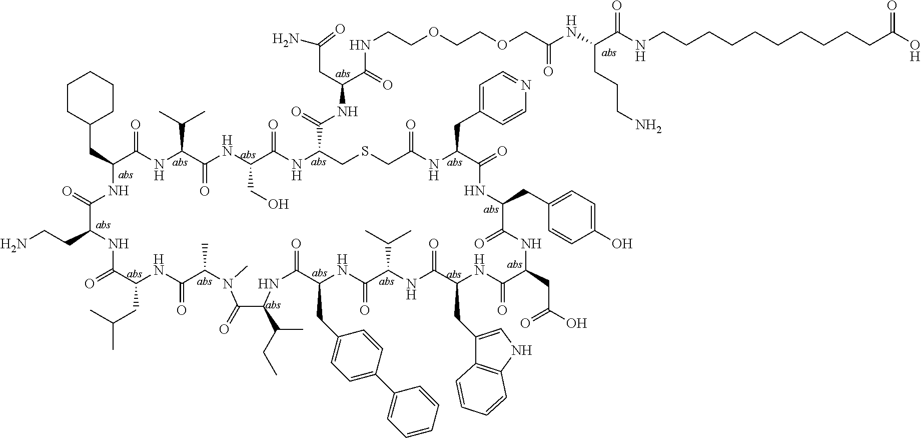 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CC(N)=O)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2078 was prepared on a 50 μmol scale. The yield of the product was 20.1 mg, and its estimated purity by LCMS analysis was 100%. Analysis condition A: Retention time=1.57 min; ESI-MS(+) m/z [M+3H]³⁺: 802.2.

### Preparation of Example 2079

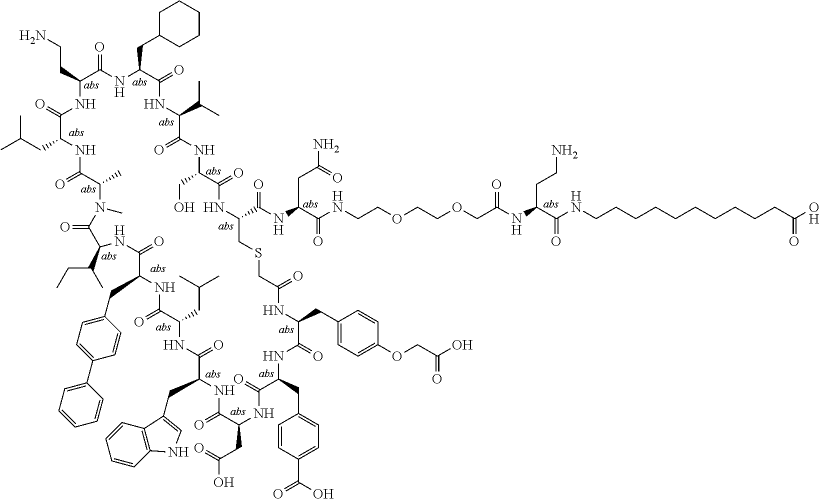 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCC(=O)N[C@@H](CCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2079 was prepared on a 50 μmol scale. The yield of the product was 30 mg, and its estimated purity by LCMS analysis was 95.5%. Analysis condition A: Retention time=1.64 min; ESI-MS(+) m/z [M+2H]²⁺: 1252.9.

### Preparation of Example 2080

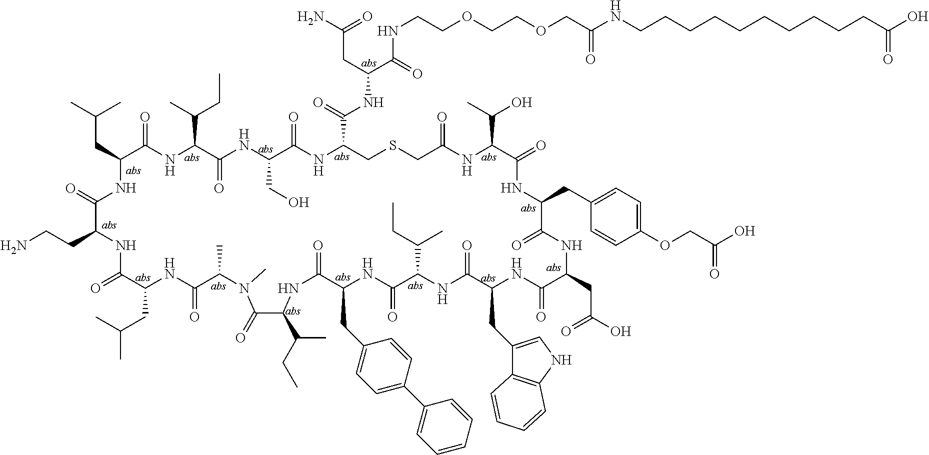 [SMILES: CCC(C)[C@@H]1NC(=O)[C@H](CC(C)C)NC(=O)[C@H](CCN)NC(=O)[C@@H](CC(C)C)NC(=O)[C@H](C)N(C)C(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)NCCCCCCCCCCC(=O)O)NC(=O)[C@H](CO)NC1=O]

Example 2080 was prepared on a 50 μmol scale. The yield of the product was 23 mg, and its estimated purity by LCMS analysis was 96.1%. Analysis condition B: Retention time=1.85 min; ESI-MS(+) m/z [M+2H]²⁺: 1145.1.

### Preparation of Example 2081

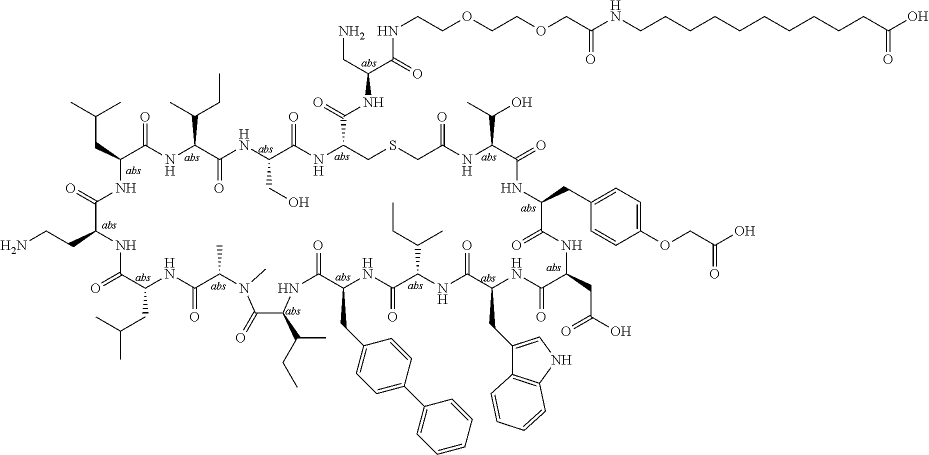 [SMILES: CCC(C)[C@@H]1NC(=O)[C@H](CC(C)C)NC(=O)[C@H](CCN)NC(=O)[C@@H](CC(C)C)NC(=O)[C@H](C)N(C)C(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CN)C(=O)NCCOCCOCC(=O)NCCCCCCCCCCC(=O)O)NC(=O)[C@H](CO)NC1=O]

Example 2081 was prepared on a 50 μmol scale. The yield of the product was 7.8 mg, and its estimated purity by LCMS analysis was 97.5%. Analysis condition B: Retention time=1.69 min; ESI-MS(+) m/z [M+2H]²⁺: 1131.1.

### Preparation of Example 2082

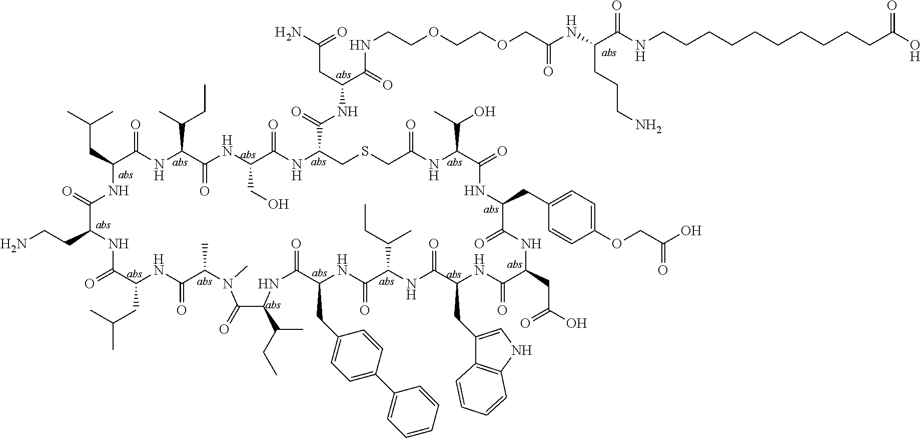 [SMILES: CCC(C)[C@@H]1NC(=O)[C@H](CC(C)C)NC(=O)[C@H](CCN)NC(=O)[C@@H](CC(C)C)NC(=O)[C@H](C)N(C)C(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N[C@@H](CCCN)C(=O)NCCCCCCCCCCC(=O)O)NC(=O)[C@H](CO)NC1=O]

Example 2082 was prepared on a 50 μmol scale. The yield of the product was 23.3 mg, and its estimated purity by LCMS analysis was 99.1%. Analysis condition A: Retention time=1.58 min; ESI-MS(+) m/z [M+2H]²⁺: 1202.2.

### Preparation of Example 2083

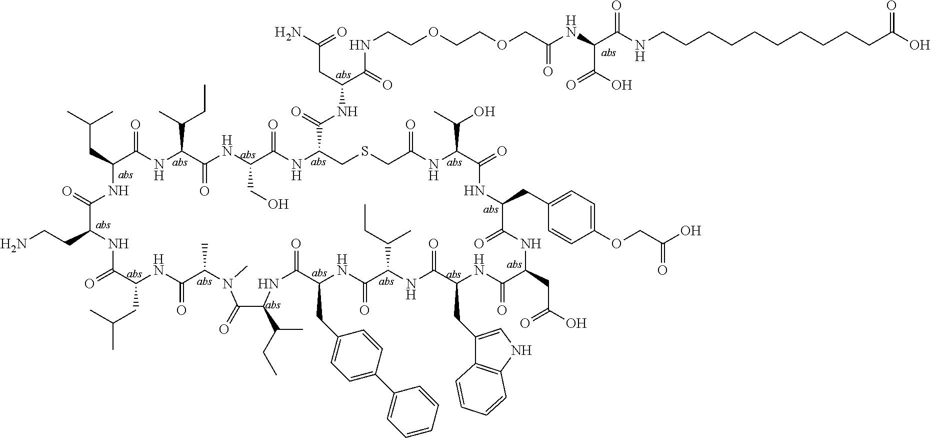 [SMILES: CCC(C)[C@@H]1NC(=O)[C@H](CC(C)C)NC(=O)[C@H](CCN)NC(=O)[C@@H](CC(C)C)NC(=O)[C@H](C)N(C)C(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N[C@H](C(=O)O)C(=O)NCCCCCCCCCCC(=O)O)NC(=O)[C@H](CO)NC1=O]

Example 2083 was prepared on a 50 μmol scale. The yield of the product was 29.7 mg, and its estimated purity by LCMS analysis was 96.2%. Analysis condition A: Retention time=1.4 min; ESI-MS(+) m/z [M+2H]²⁺: 806.5.

### Preparation of Example 2084

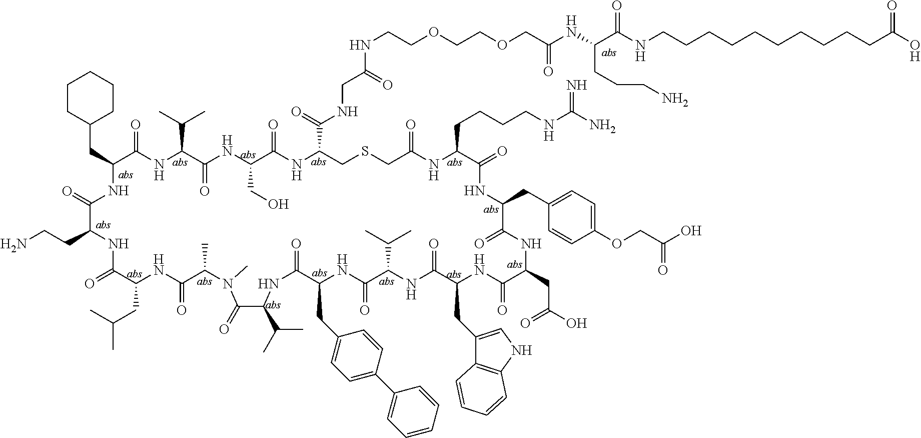 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2084 was prepared on a 50 μmol scale. The yield of the product was 23.1 mg, and its estimated purity by LCMS analysis was 96.7%. Analysis condition B: Retention time=1.64 min; ESI-MS(+) m/z [M+3H]³⁺: 805.

### Preparation of Example 2085

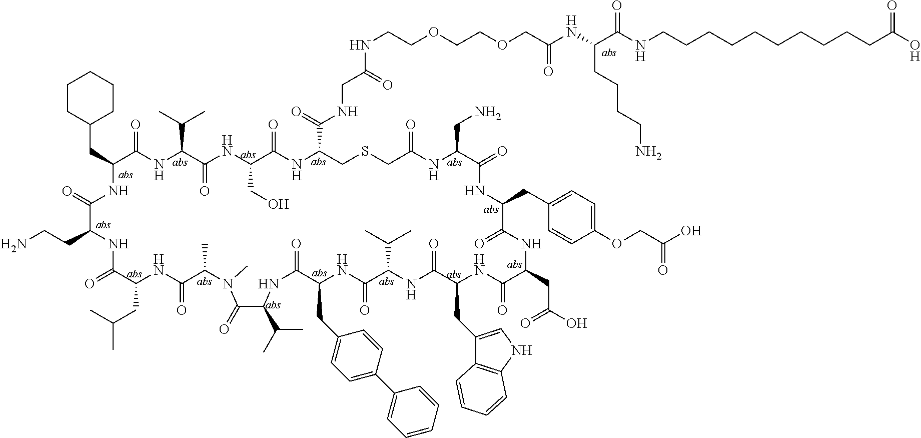 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2085 was prepared on a 50 μmol scale. The yield of the product was 12.2 mg, and its estimated purity by LCMS analysis was 95.9%. Analysis condition A: Retention time=1.66 min; ESI-MS(+) m/z [M+2H]²⁺: 1172.

### Preparation of Example 2086

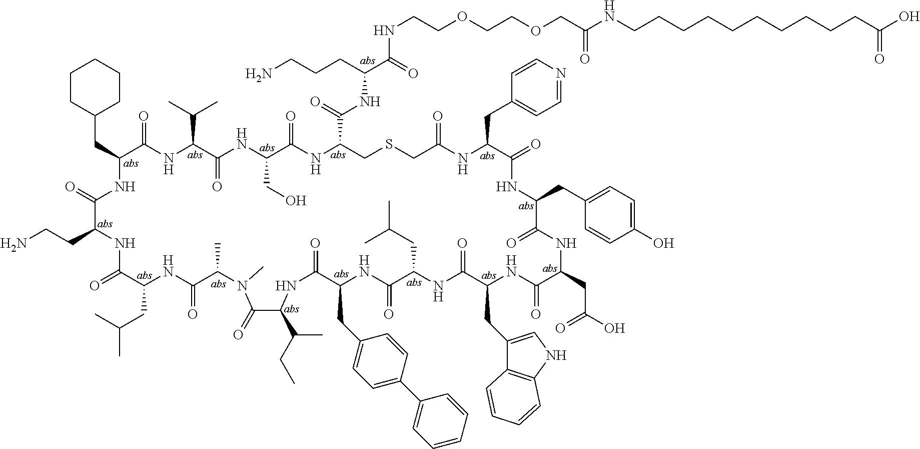 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2086 was prepared on a 50 μmol scale. The yield of the product was 14.2 mg, and its estimated purity by LCMS analysis was 99%. Analysis condition B: Retention time=1.73 min; ESI-MS(+) m/z [M+2H]²⁺: 1152.1.

### Preparation of Example 2087

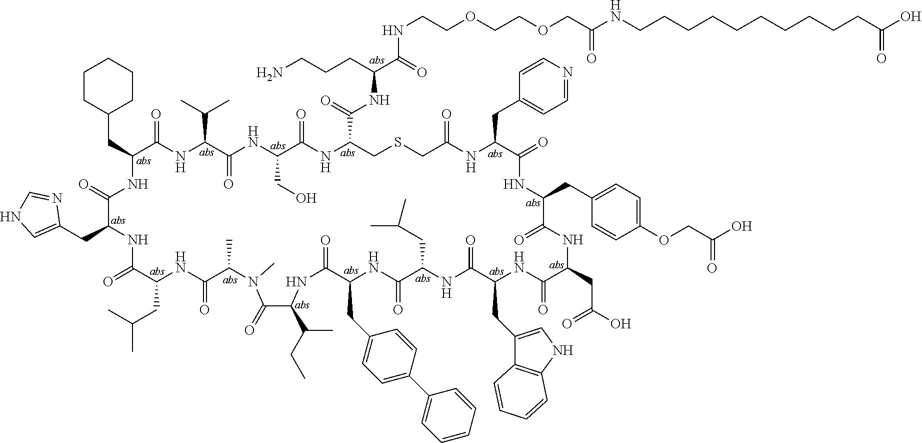 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](Cc2c[nH]cn2)NC(=O)[C@@H](CC(C)C)NC(=O)[C@H](C)N(C)C1=O]

Example 2087 was prepared on a 50 μmol scale. The yield of the product was 9.8 mg, and its estimated purity by LCMS analysis was 95.7%. Analysis condition B: Retention time=1.79 min; ESI-MS(+) m/z [M+2H]²⁺: 1199.4.

### Preparation of Example 2088

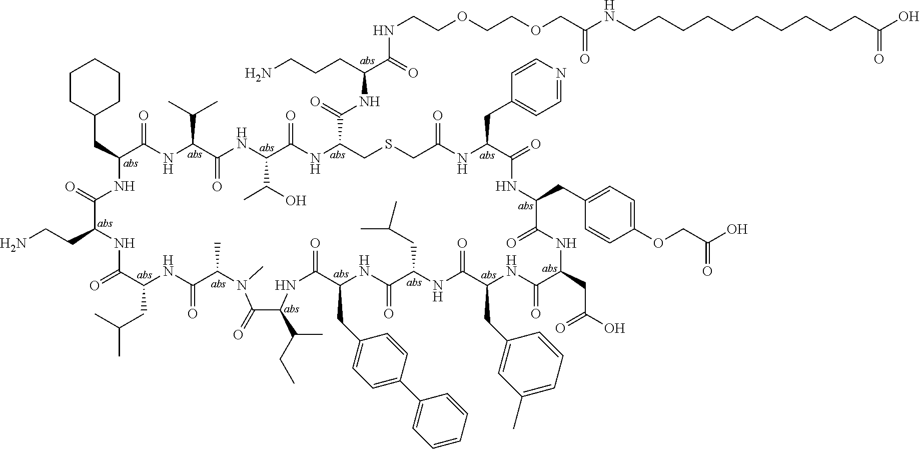 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2088 was prepared on a 50 μmol scale. The yield of the product was 13.2 mg, and its estimated purity by LCMS analysis was 97.5%. Analysis condition B: Retention time=1.84 min; ESI-MS(+) m/z [M+2H]²⁺: 1176.1.

### Preparation of Example 2089

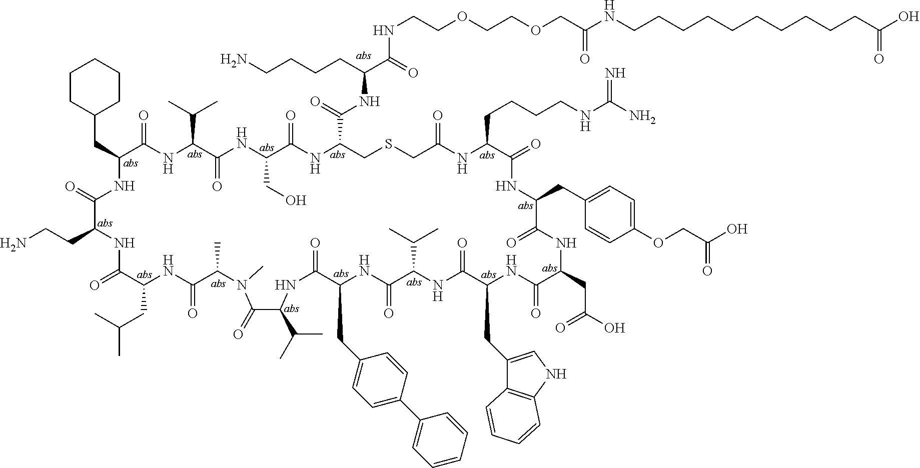 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2089 was prepared on a 50 μmol scale. The yield of the product was 25.1 mg, and its estimated purity by LCMS analysis was 97.4%. Analysis condition B: Retention time=1.76 min; ESI-MS(+) m/z [M+2H]²⁺: 791.1.

### Preparation of Example 2090

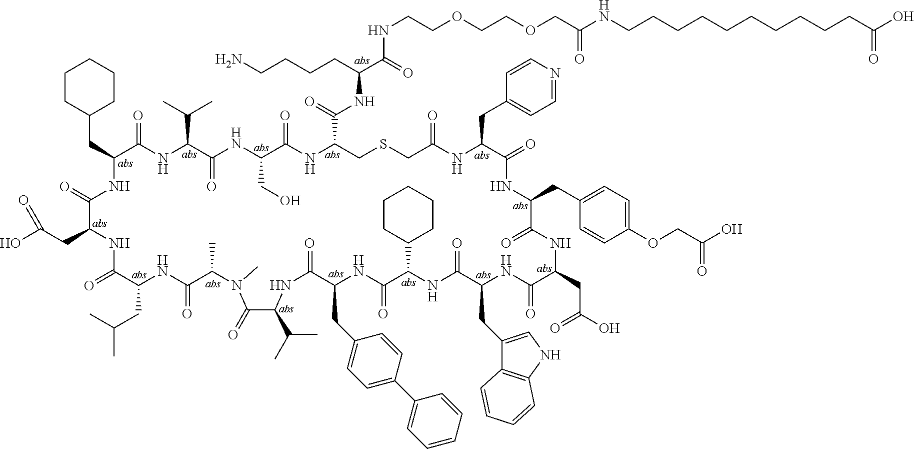 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(=O)O)NC1=O]

Example 2090 was prepared on a 50 μmol scale. The yield of the product was 18.9 mg, and its estimated purity by LCMS analysis was 95.2%. Analysis condition A: Retention time=1.66 min; ESI-MS(+) m/z [M+2H]²⁺: 1202.1.

### Preparation of Example 2091

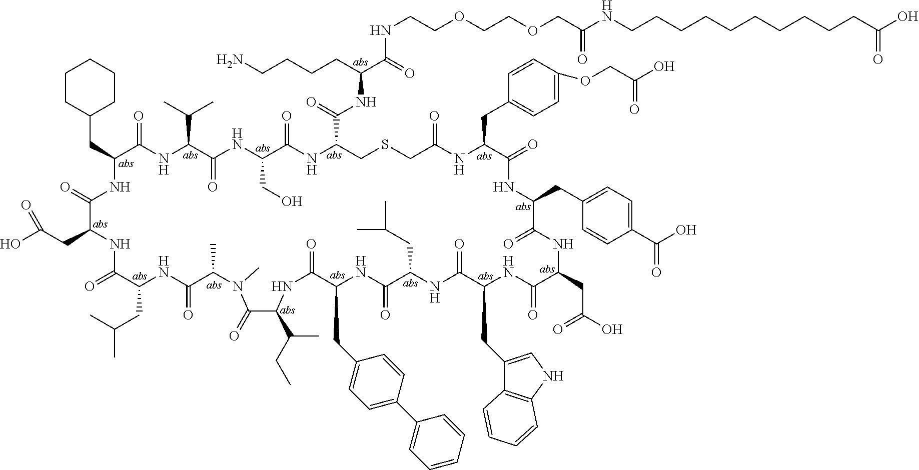 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(=O)O)NC(=O)[C@@H](CC(C)C)NC(=O)[C@H](C)N(C)C1=O]

Example 2091 was prepared on a 50 μmol scale. The yield of the product was 17.8 mg, and its estimated purity by LCMS analysis was 98.5%. Analysis condition B: Retention time=2.17 min; ESI-MS(+) m/z [M+2H]²⁺: 1210.

### Preparation of Example 2092

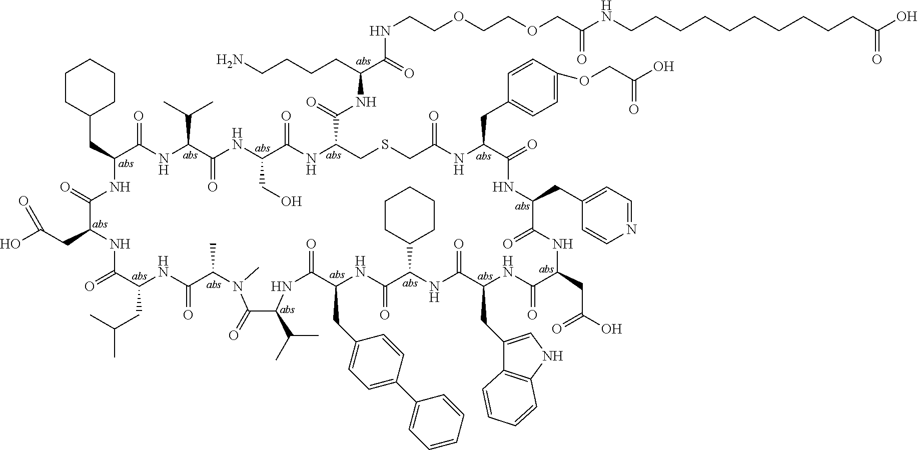 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(=O)O)NC1=O]

Example 2092 was prepared on a 50 μmol scale. The yield of the product was 20.3 mg, and its estimated purity by LCMS analysis was 95.6%. Analysis condition A: Retention time=1.7 min; ESI-MS(+) m/z [M+2H]²⁺: 1601.8.

### Preparation of Example 2093

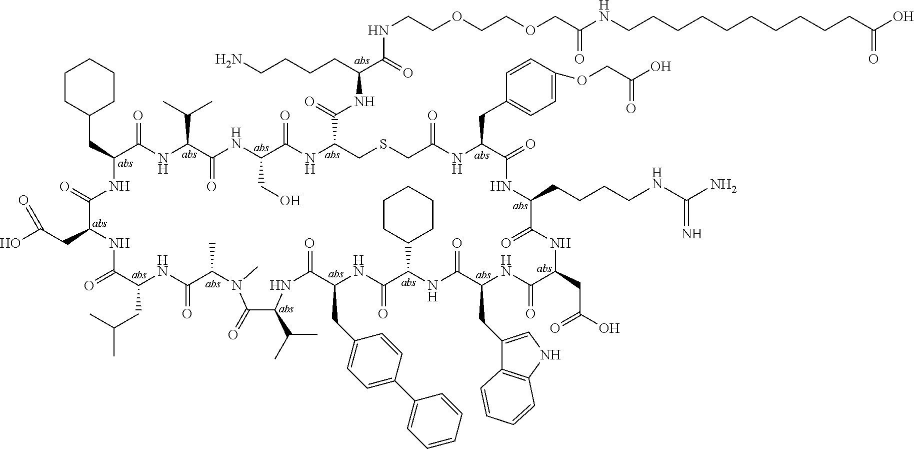 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CC(=O)O)NC1=O]

Example 2093 was prepared on a 50 μmol scale. The yield of the product was 15.7 mg, and its estimated purity by LCMS analysis was 96.5%. Analysis condition A: Retention time=1.74 min; ESI-MS(+) m/z [M+2H]²⁺: 1213.1.

### Preparation of Example 2094

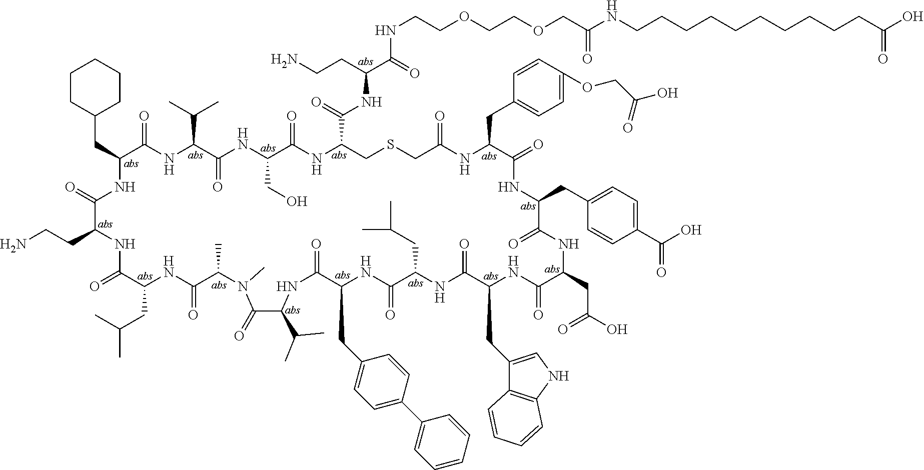 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2094 was prepared on a 50 μmol scale. The yield of the product was 18.1 mg, and its estimated purity by LCMS analysis was 99%. Analysis condition A: Retention time=1.59 min; ESI-MS(+) m/z [M+2H]²⁺: 1189.2.

### Preparation of Example 2095

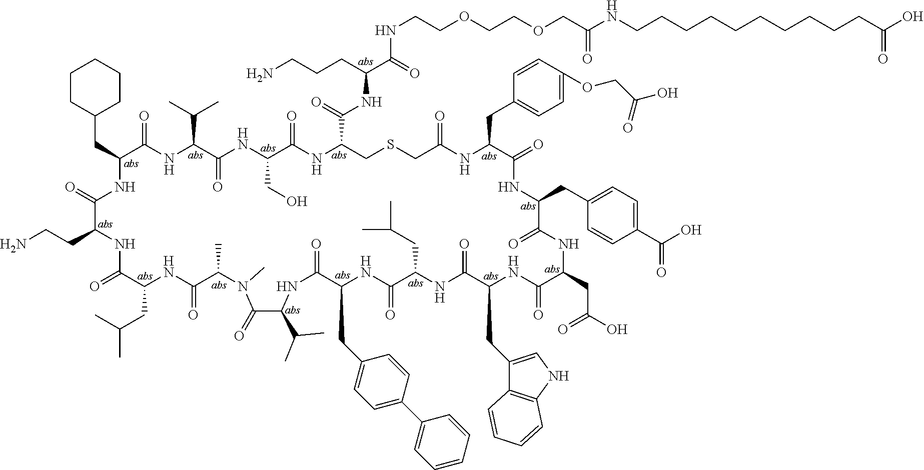 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2095 was prepared on a 50 μmol scale. The yield of the product was 22.4 mg, and its estimated purity by LCMS analysis was 95.4%. Analysis condition A: Retention time=1.63 min; ESI-MS(+) m/z [M+2H]²⁺: 1196.2.

### Preparation of Example 2096

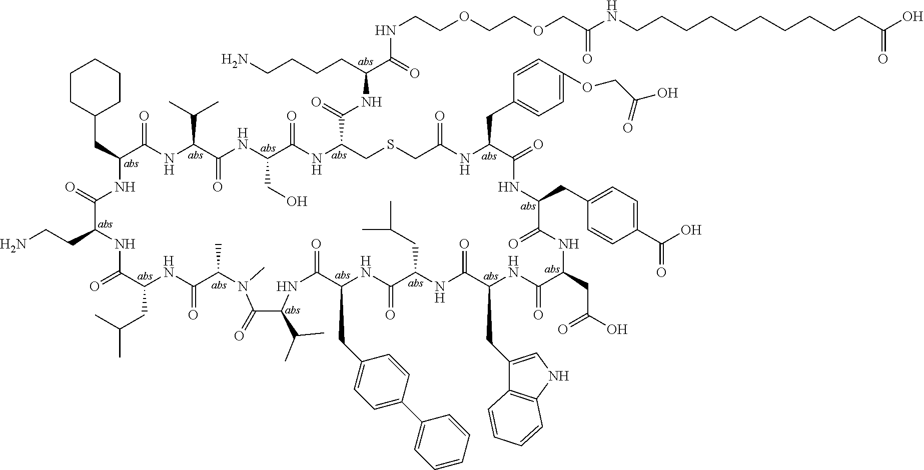 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2096 was prepared on a 50 μmol scale. The yield of the product was 23.7 mg, and its estimated purity by LCMS analysis was 99.2%. Analysis condition A: Retention time=1.65 min; ESI-MS(+) m/z [M+2H]²⁺: 1203.

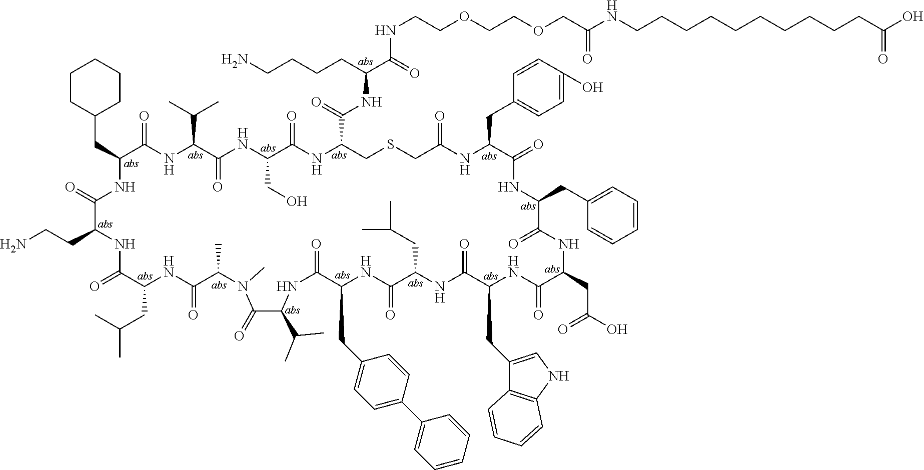 [SMILES: CC(C)C[C@@H]1NC(=O)[C@H](Cc2c[nH]c3ccccc23)NC(=O)[C@H](CC(=O)O)NC(=O)[C@H](Cc2ccccc2)NC(=O)[C@H](Cc2ccc(O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2097 was prepared on a 50 μmol scale. The yield of the product was 26.4 mg, and its estimated purity by LCMS analysis was 98.5%. Analysis condition A: Retention time=1.92 min; ESI-MS(+) m/z [M+2H]²⁺: 1152.1.

### Preparation of Example 2098

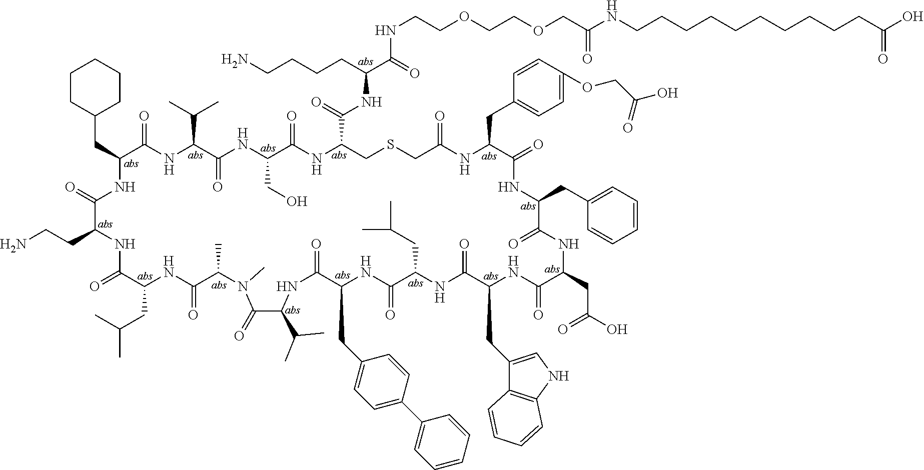 [SMILES: CC(C)C[C@@H]1NC(=O)[C@H](Cc2c[nH]c3ccccc23)NC(=O)[C@H](CC(=O)O)NC(=O)[C@H](Cc2ccccc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2098 was prepared on a 50 μmol scale. The yield of the product was 36.2 mg, and its estimated purity by LCMS analysis was 99.2%. Analysis condition A: Retention time=1.86 min; ESI-MS(+) m/z [M+2H]²⁺: 1181.2.

### Preparation of Example 2099

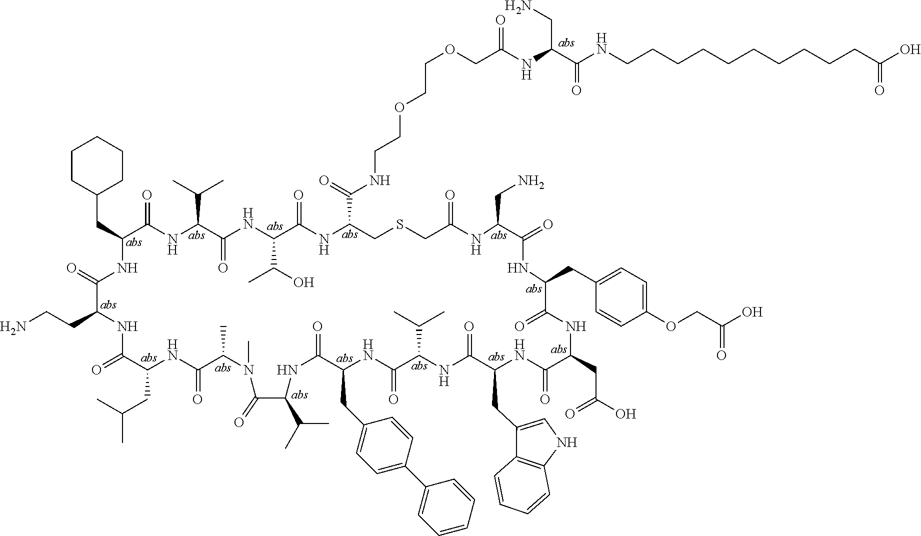 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2099 was prepared on a 50 μmol scale. The yield of the product was 32.7 mg, and its estimated purity by LCMS analysis was 97.1%. Analysis condition A: Retention time=1.67, 1.72 min; ESI-MS(+) m/z [M+3H]³⁺: 753.22, 753.06.

### Preparation of Example 2100

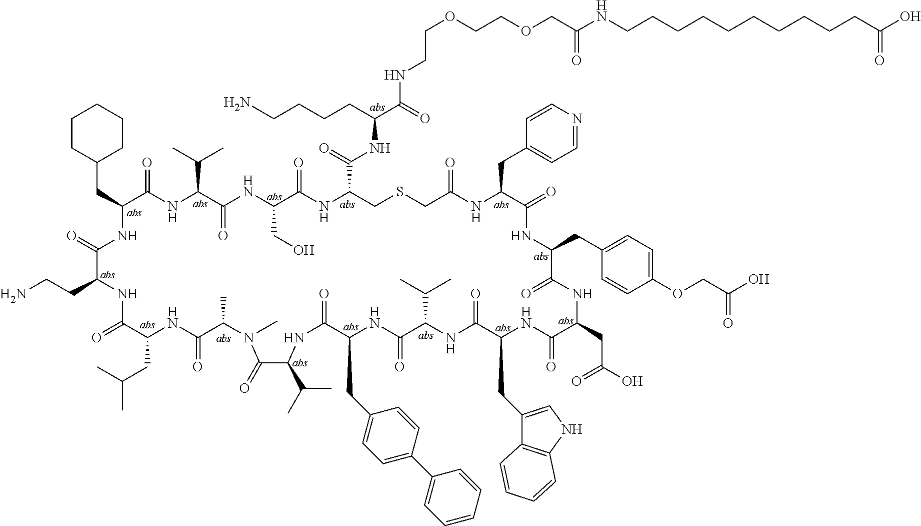 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2100 was prepared on a 50 μmol scale. The yield of the product was 47.2 mg, and its estimated purity by LCMS analysis was 97.2%. Analysis condition A: Retention time=1.63 min; ESI-MS(+) m/z [M+3H]³⁺: 783.6.

### Preparation of Example 2101

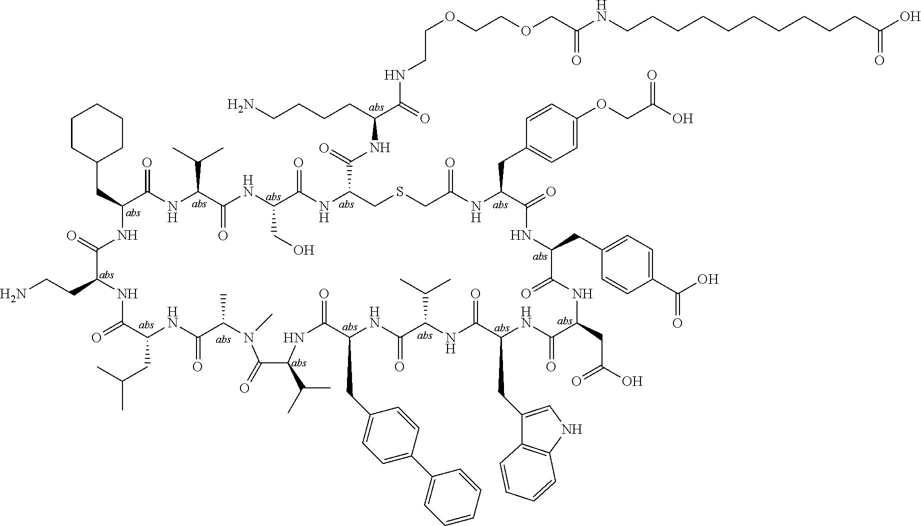 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2101 was prepared on a 50 μmol scale. The yield of the product was 17.8 mg, and its estimated purity by LCMS analysis was 100%. Analysis condition A: Retention time=1.4 min; ESI-MS(+) m/z [M+2H]²⁺: 1196.

### Preparation of Example 2102

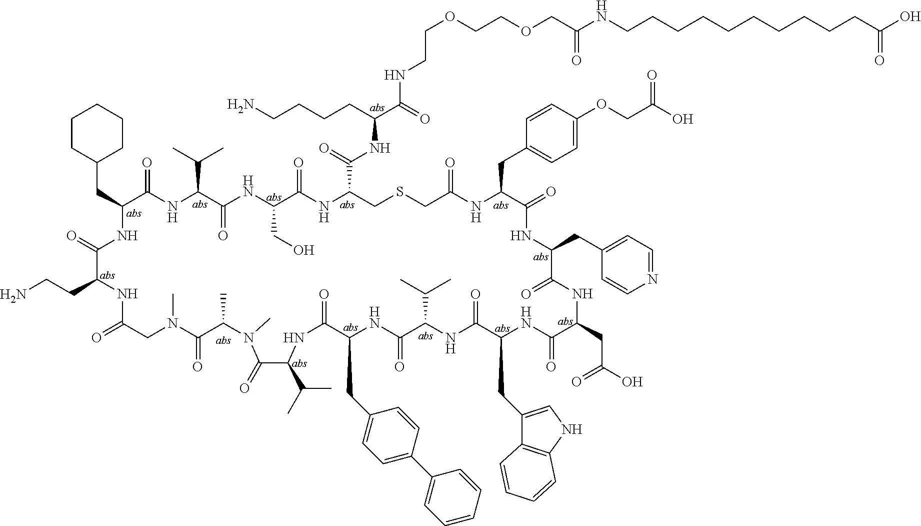 [SMILES: CC(C)[C@@H]1NC(=O)[C@H](CC2CCCCC2)NC(=O)[C@H](CCN)NC(=O)CN(C)C(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC1=O]

Example 2102 was prepared on a 50 μmol scale. The yield of the product was 16.9 mg, and its estimated purity by LCMS analysis was 95.1%. Analysis condition A: Retention time=1.51 min; ESI-MS(+) m/z [M+2H]²⁺: 1154.1.

### Preparation of Example 2103

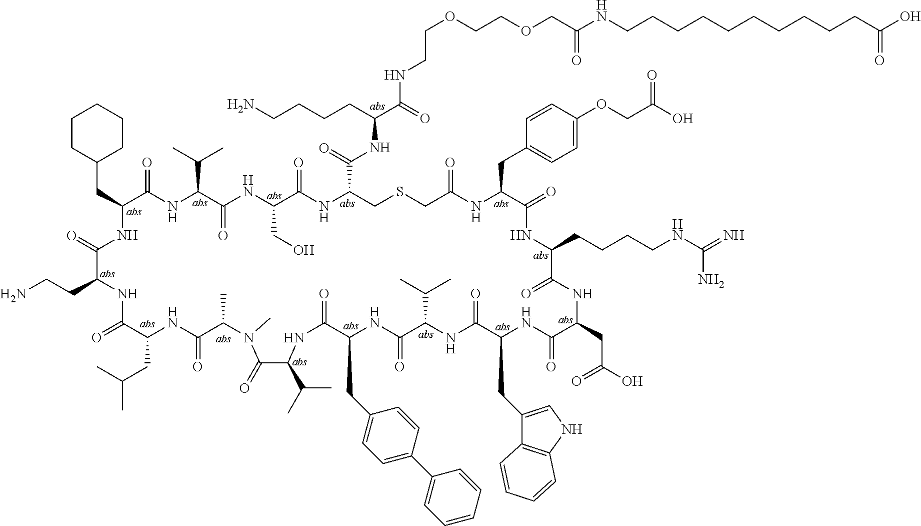 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2103 was prepared on a 50 μmol scale. The yield of the product was 31 mg, and its estimated purity by LCMS analysis was 95.5%. Analysis condition A: Retention time=1.46 min; ESI-MS(+) m/z [M+3H]³⁺: 791.1.

### Preparation of Example 2104

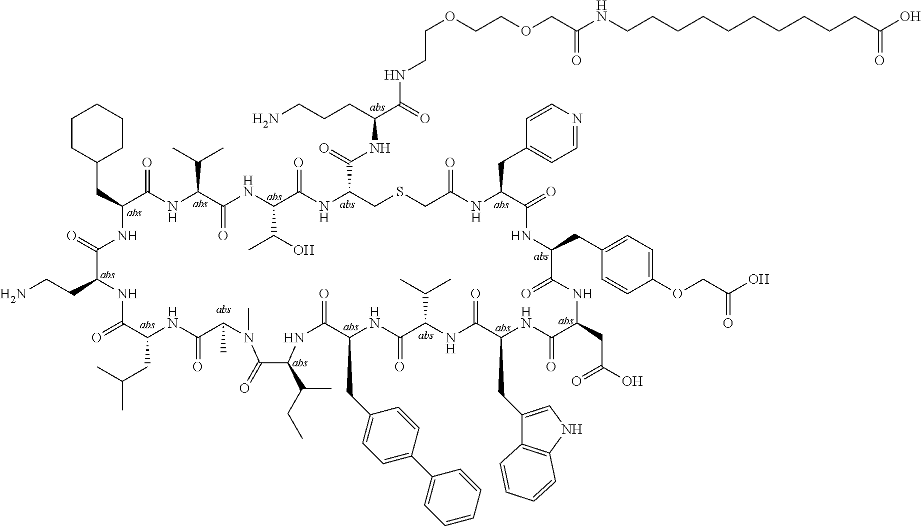 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2104 was prepared on a 50 μmol scale. The yield of the product was 43.4 mg, and its estimated purity by LCMS analysis was 100%. Analysis condition A: Retention time=1.69 min; ESI-MS(+) m/z [M+3H]³⁺: 788.2.

### Preparation of Example 2105

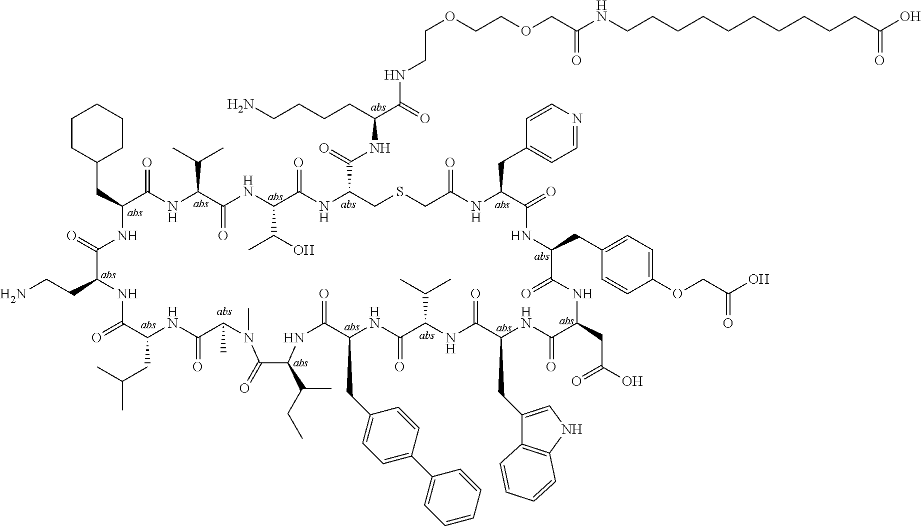 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2105 was prepared on a 50 μmol scale. The yield of the product was 27 mg, and its estimated purity by LCMS analysis was 99.2%. Analysis condition B: Retention time=1.76 min; ESI-MS(+) m/z [M+3H]⁺: 792.4.

### Preparation of Example 2106

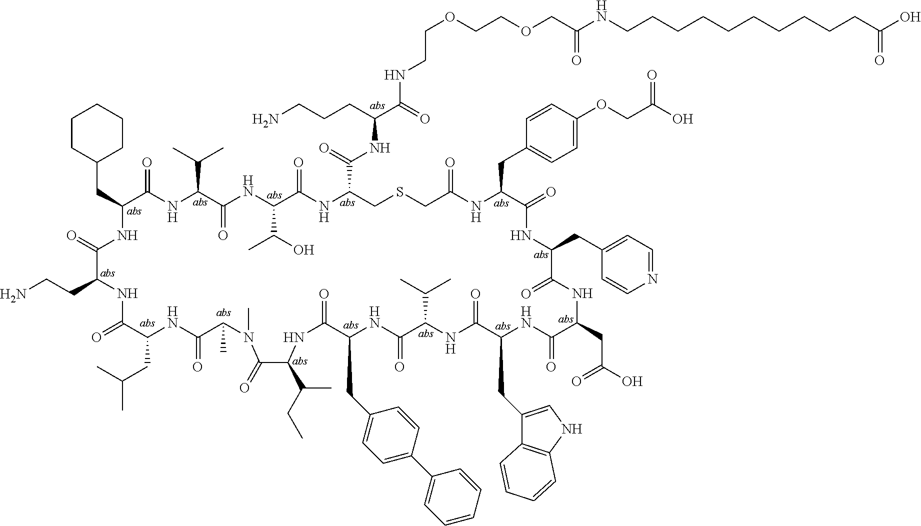 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2106 was prepared on a 50 μmol scale. The yield of the product was 66.1 mg, and its estimated purity by LCMS analysis was 95.4%. Analysis condition B: Retention time=1.76 min; ESI-MS(+) m/z [M+3H]⁺: 788.1.

### Preparation of Example 2107

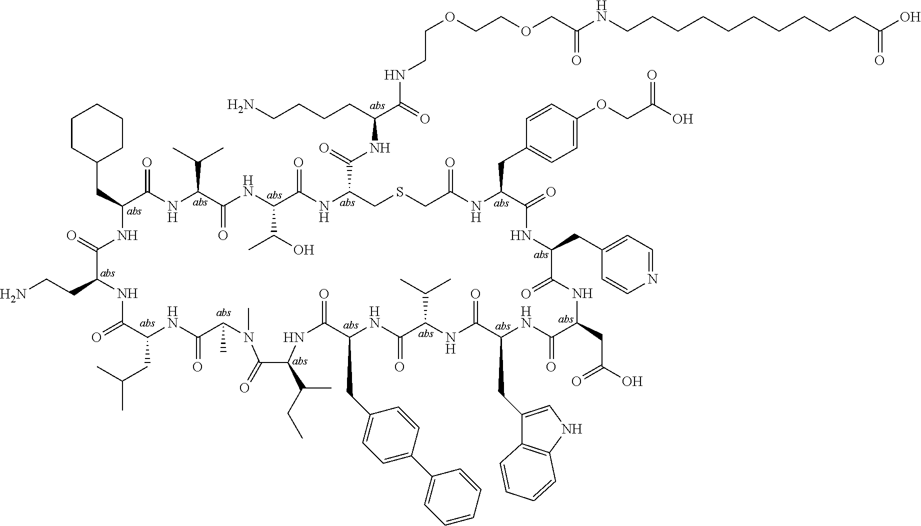 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2107 was prepared on a 50 μmol scale. The yield of the product was 38 mg, and its estimated purity by LCMS analysis was 96.8%. Analysis condition B: Retention time=1.74 min; ESI-MS(+) m/z [M+3H]³⁺: 792.6.

### Preparation of Example 2108

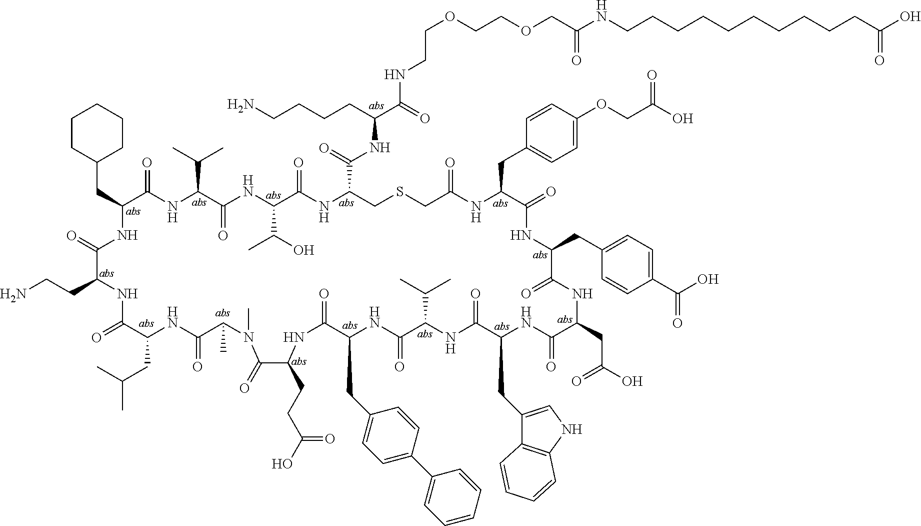 [SMILES: CC(C)C[C@H]1NC(=O)[C@H](C)N(C)C(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2108 was prepared on a 50 μmol scale. The yield of the product was 73.3 mg, and its estimated purity by LCMS analysis was 96.8%. Analysis condition A: Retention time=1.35 min; ESI-MS(+) m/z [M+2H]²⁺: 1218.

### Preparation of Example 2109

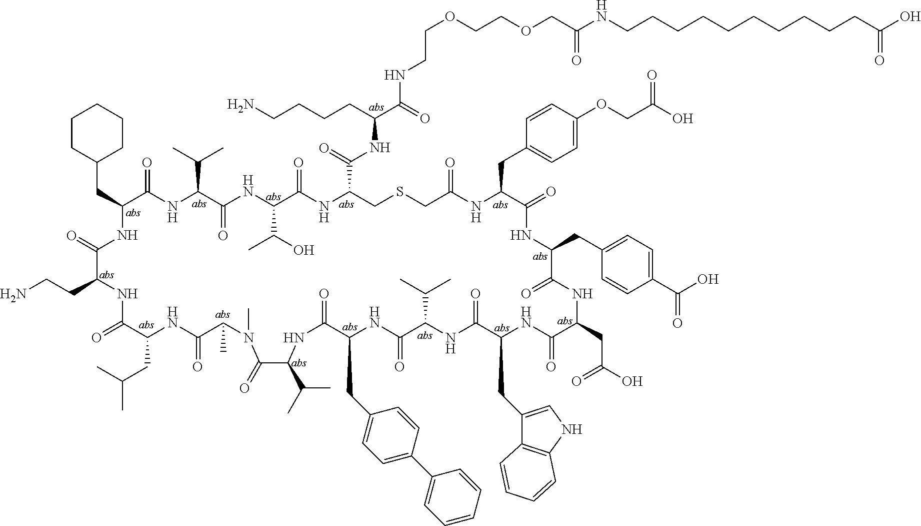 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2109 was prepared on a 50 μmol scale. The yield of the product was 60.1 mg, and its estimated purity by LCMS analysis was 99.4%. Analysis condition B: Retention time=1.82 min; ESI-MS(+) m/z [M+2H]²⁺: 1203.2.

### Preparation of Example 2110

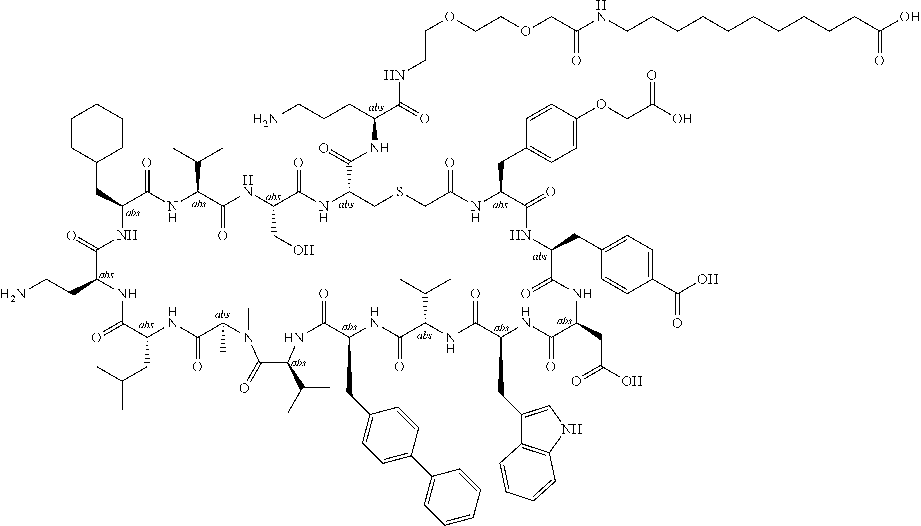 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2110 was prepared on a 50 μmol scale. The yield of the product was 45.5 mg, and its estimated purity by LCMS analysis was 97.1%. Analysis condition A: Retention time=1.38 min; ESI-MS(+) m/z [M+2H]²⁺: 1189.1.

### Preparation of Example 2111

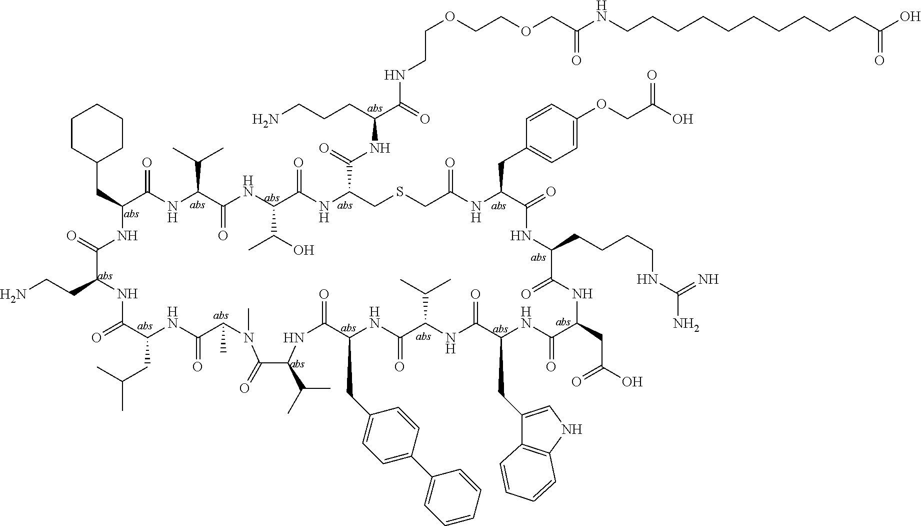 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2111 was prepared on a 50 μmol scale. The yield of the product was 46.2 mg, and its estimated purity by LCMS analysis was 100%. Analysis condition A: Retention time=1.65 min; ESI-MS(+) m/z [M+2H]²⁺: 1185.2.

### Preparation of Example 2112

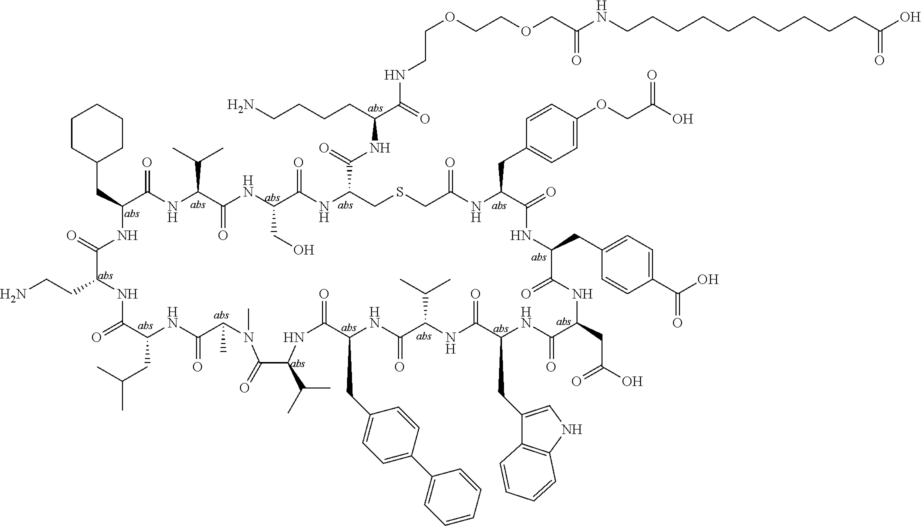 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@@H](CCN)NC1=O]

Example 2112 was prepared on a 50 μmol scale. The yield of the product was 59.2 mg, and its estimated purity by LCMS analysis was 95.3%. Analysis condition A: Retention time=1.55 min; ESI-MS(+) m/z [M+2H]²⁺: 1196.1.

### Preparation of Example 2113

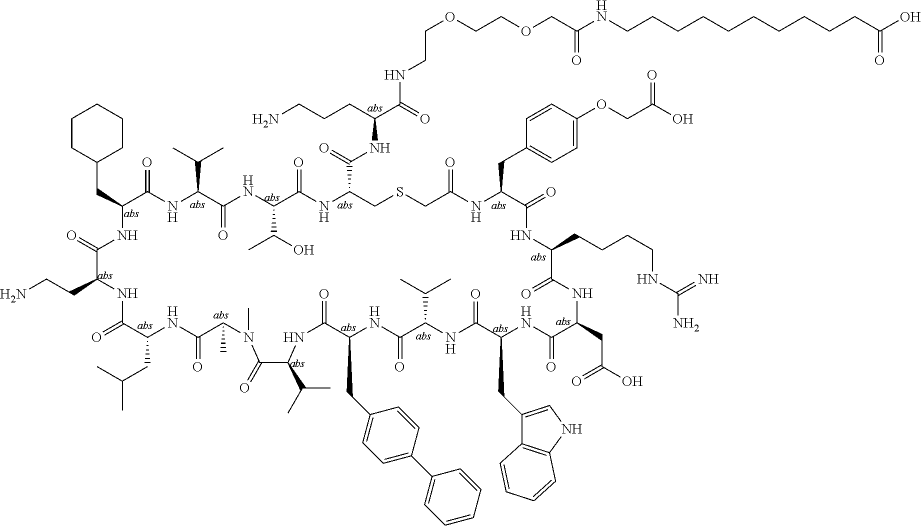 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2113 was prepared on a 50 μmol scale. The yield of the product was 42.6 mg, and its estimated purity by LCMS analysis was 97.1%. Analysis condition B: Retention time=1.73 min; ESI-MS(+) m/z [M+3H]³⁺: 795.1.

### Preparation of Example 2114

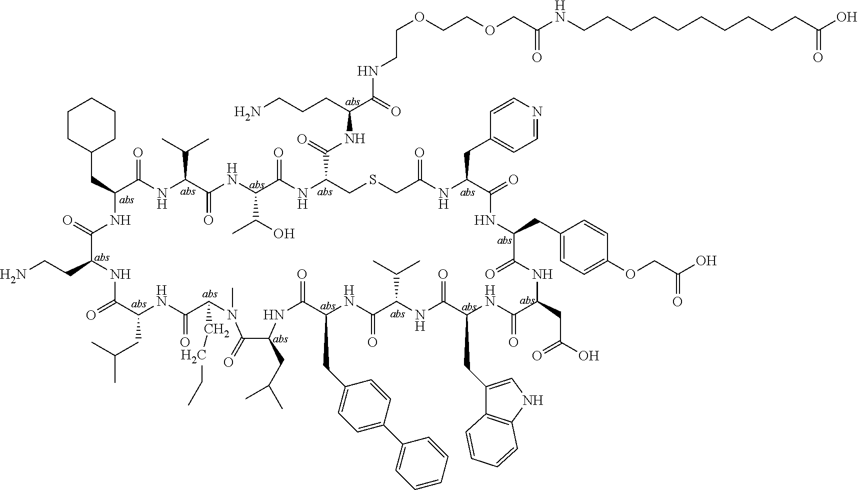 [SMILES: CCCC[C@H]1C(=O)N[C@H](CC(C)C)C(=O)N[C@@H](CCN)C(=O)N[C@@H](CC2CCCCC2)C(=O)N[C@@H](C(C)C)C(=O)N[C@@H](C(C)O)C(=O)N[C@H](C(=O)N[C@@H](CCCN)C(=O)NCCOCCOCC(=O)NCCCCCCCCCCC(=O)O)CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CC(C)C)C(=O)N1C]

Example 2114 was prepared on a 50 μmol scale. The yield of the product was 56.7 mg, and its estimated purity by LCMS analysis was 96.1%. Analysis condition B: Retention time=1.96 min; ESI-MS(+) m/z [M+2H]²⁺: 1202.9.

### Preparation of Example 2115

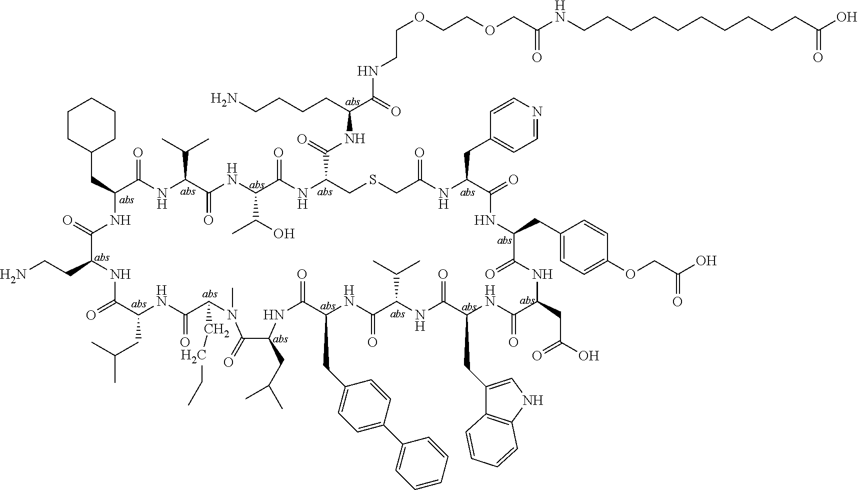 [SMILES: CCCC[C@H]1C(=O)N[C@H](CC(C)C)C(=O)N[C@@H](CCN)C(=O)N[C@@H](CC2CCCCC2)C(=O)N[C@@H](C(C)C)C(=O)N[C@@H](C(C)O)C(=O)N[C@H](C(=O)N[C@@H](CCCCN)C(=O)NCCOCCOCC(=O)NCCCCCCCCCCC(=O)O)CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CC(C)C)C(=O)N1C]

Example 2115 was prepared on a 50 μmol scale. The yield of the product was 78.3 mg, and its estimated purity by LCMS analysis was 95.5%. Analysis condition A: Retention time=1.83 min; ESI-MS(+) m/z [M+2H]²⁺: 1210.

### Preparation of Example 2116 A174D-035-11

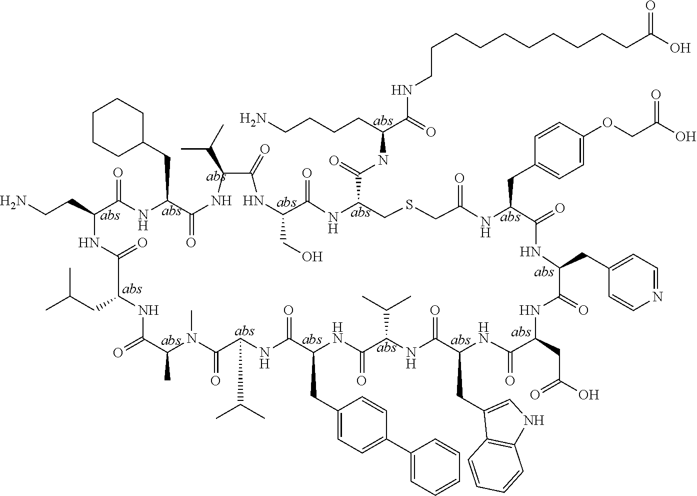 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)C[C@H](CO)NC(=O)[C@H](C(C)C)NC(=O)[C@H](CC2CCCCC2)NC(=O)[C@H](CCN)NC1=O]

Example 2116 was prepared on a 50 μmol scale. The yield of the product was 15.2 mg, and its estimated purity by LCMS analysis was 96.7%. Analysis condition B: Retention time=1.82 min; ESI-MS(+) m/z [M+2H]²⁺: 1109.

### Preparation of Example 2117

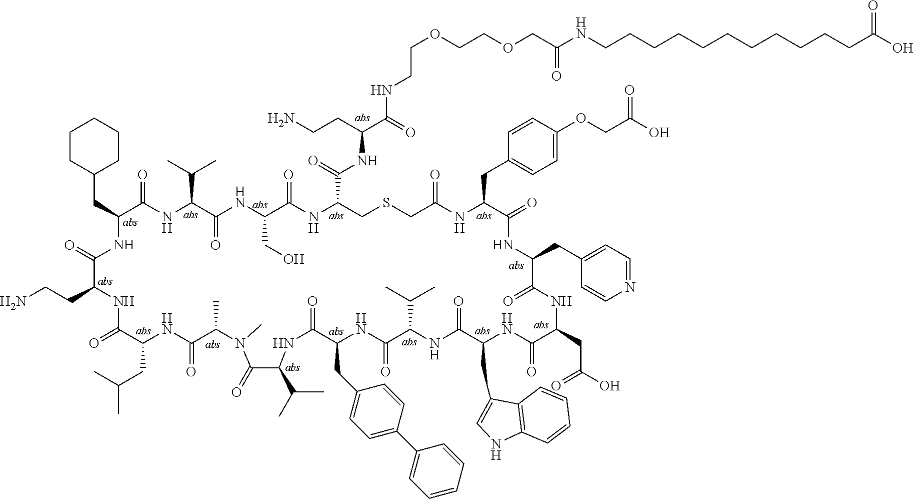 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2117 was prepared on a 50 μmol scale. The yield of the product was 8.7 mg, and its estimated purity by LCMS analysis was 95.1%. Analysis condition B: Retention time=1.75 min; ESI-MS(+) m/z [M+3H]³⁺: 779.1.

### Preparation of Example 2118

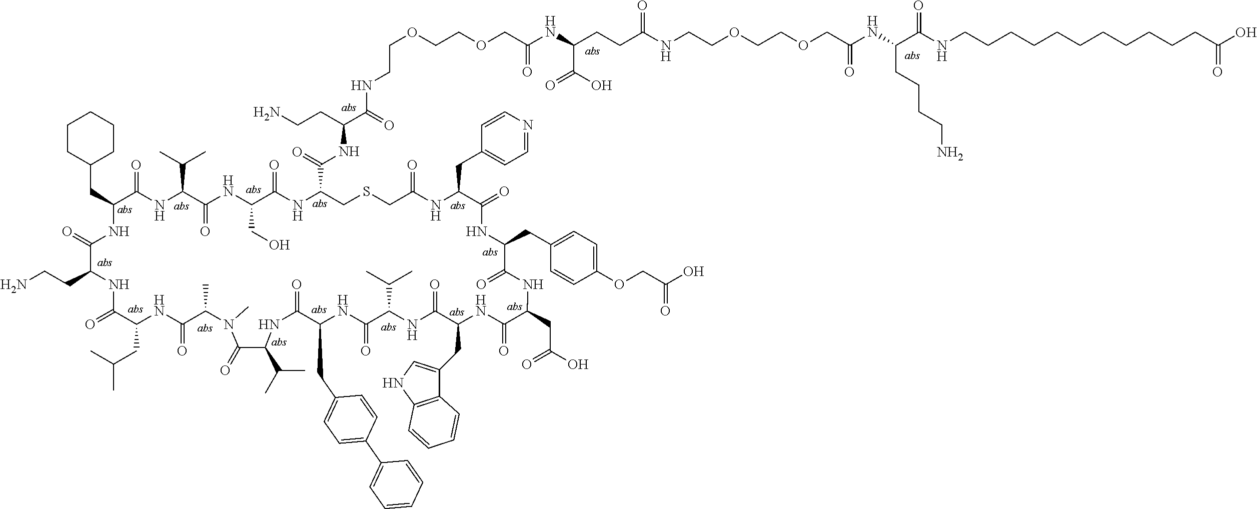 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)N[C@@H](CCC(=O)NCCOCCOCC(=O)N[C@@H](CCCCN)C(=O)NCCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC1=O]

Example 2118 was prepared on a 50 μmol scale. The yield of the product was 22.8 mg, and its estimated purity by LCMS analysis was 96.8%. Analysis condition B: Retention time=1.56 min; ESI-MS(+) m/z [M+3H]³⁺: 913.2.

### Preparation of Example 2119

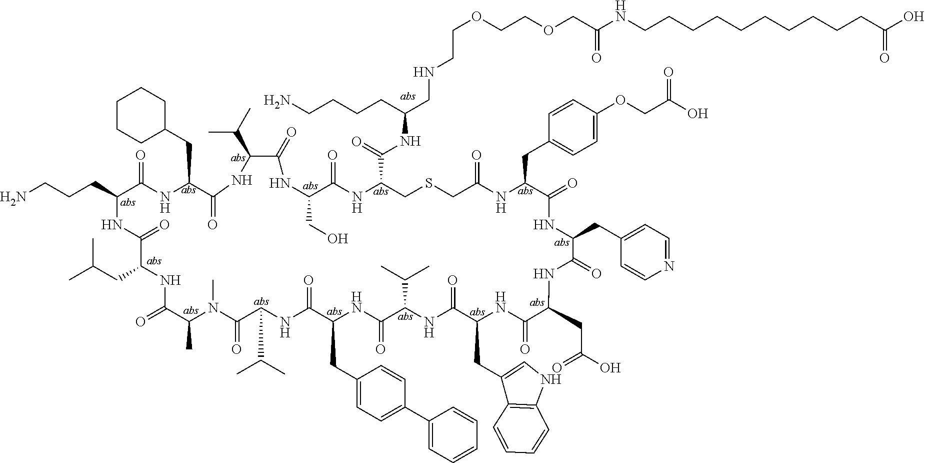 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCCN)CNCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCCN)NC1=O]

Example 2119 was prepared on a 50 μmol scale. The yield of the product was 13.8 mg, and its estimated purity by LCMS analysis was 96.9%. Analysis condition A: Retention time=1.6 min; ESI-MS(+) m/z [M+3H]³⁺: 787.8.

### Preparation of Example 2120

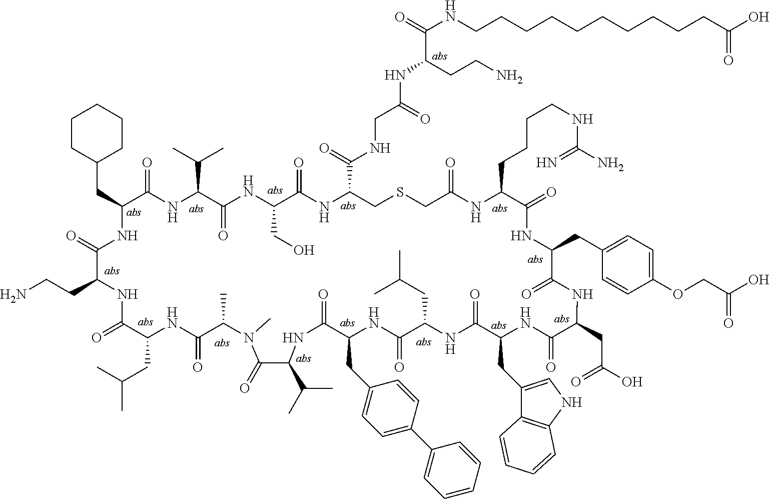 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)N[C@@H](CCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2120 was prepared on a 50 μmol scale. The yield of the product was 19.7 mg, and its estimated purity by LCMS analysis was 96.5%. Analysis condition B: Retention time=1.73 min; ESI-MS(+) m/z [M+3H]³⁺: 757.1.

### Preparation of Example 2121

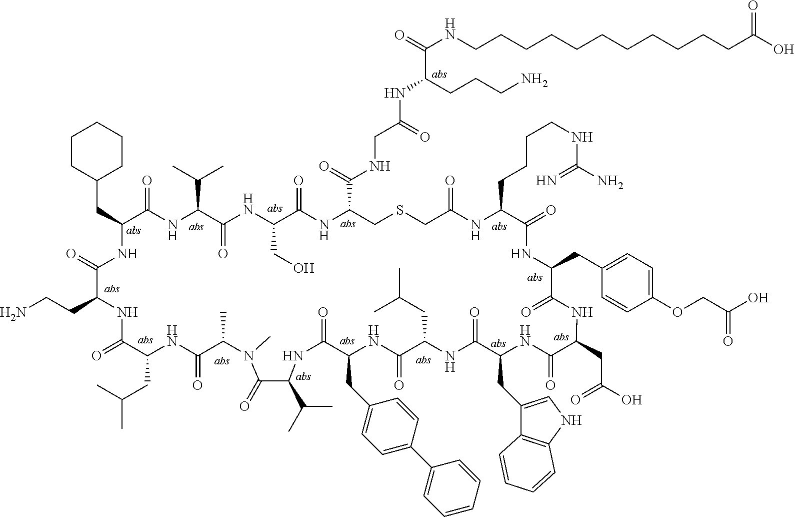 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2121 was prepared on a 50 μmol scale. The yield of the product was 29.2 mg, and its estimated purity by LCMS analysis was 95.4%. Analysis condition B: Retention time=1.76 min; ESI-MS(+) m/z [M+3H]³⁺: 766.1.

### Preparation of Example 2122

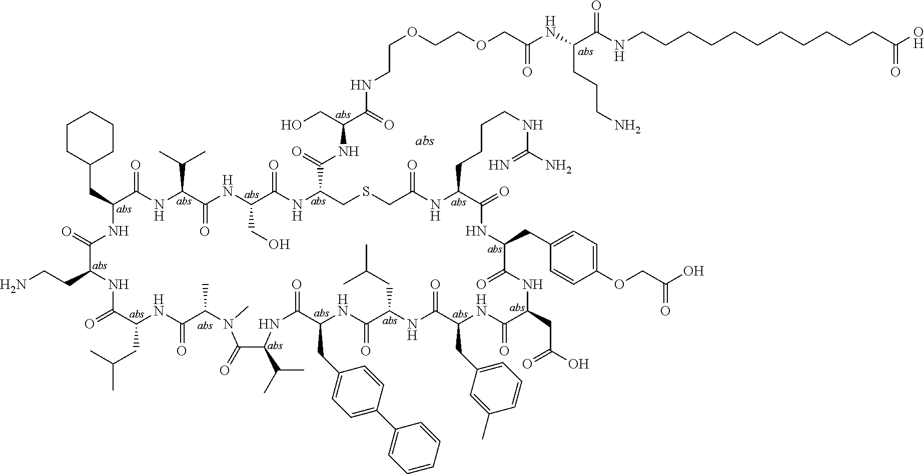 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CO)C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2122 was prepared on a 50 μmol scale. The yield of the product was 9.4 mg, and its estimated purity by LCMS analysis was 100%. Analysis condition A: Retention time=1.76 min; ESI-MS(+) m/z [M+3H]³⁺: 816.

### Preparation of Example 2123

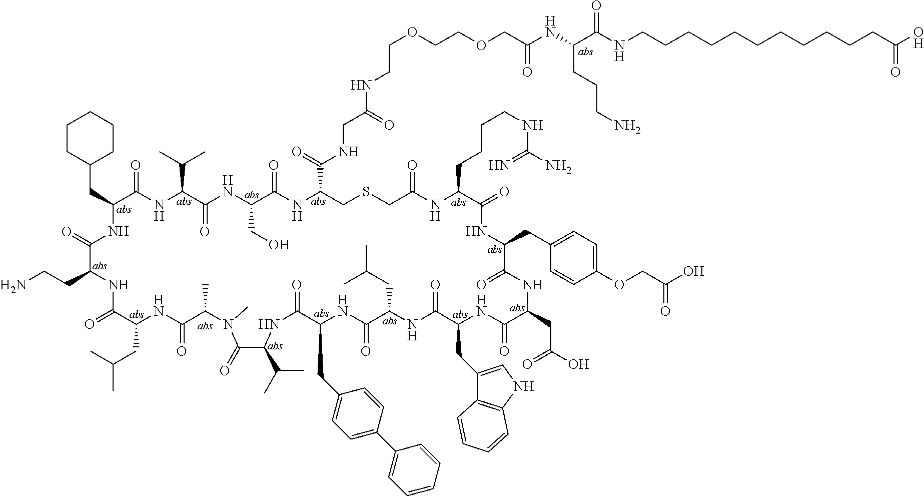 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2123 was prepared on a 50 μmol scale. The yield of the product was 28.6 mg, and its estimated purity by LCMS analysis was 100%. Analysis condition A: Retention time=1.75 min; ESI-MS(+) m/z [M+2H]²⁺: 1221.1.

### Preparation of Example 2124

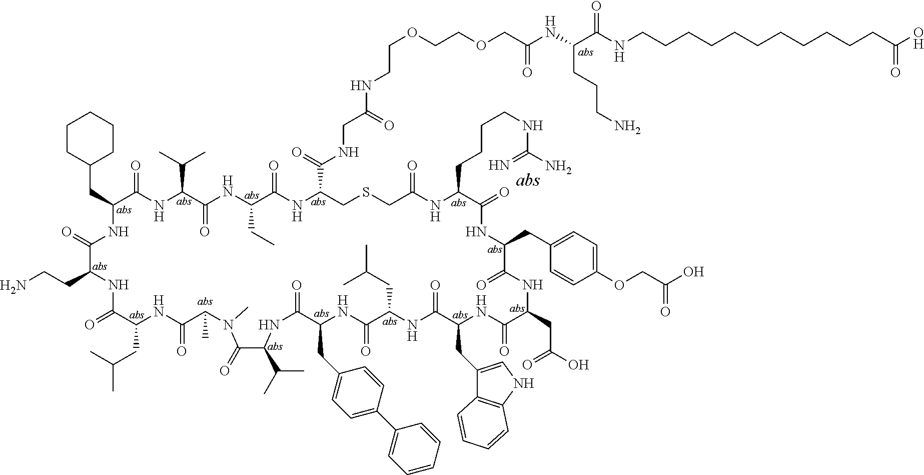 [SMILES: CC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCCC(=O)O)NC1=O]

Example 2124 was prepared on a 50 μmol scale. The yield of the product was 17.4 mg, and its estimated purity by LCMS analysis was 95.8%. Analysis condition A: Retention time=1.76 min; ESI-MS(+) m/z [M+3H]³⁺: 814.1.

### Preparation of Example 2125

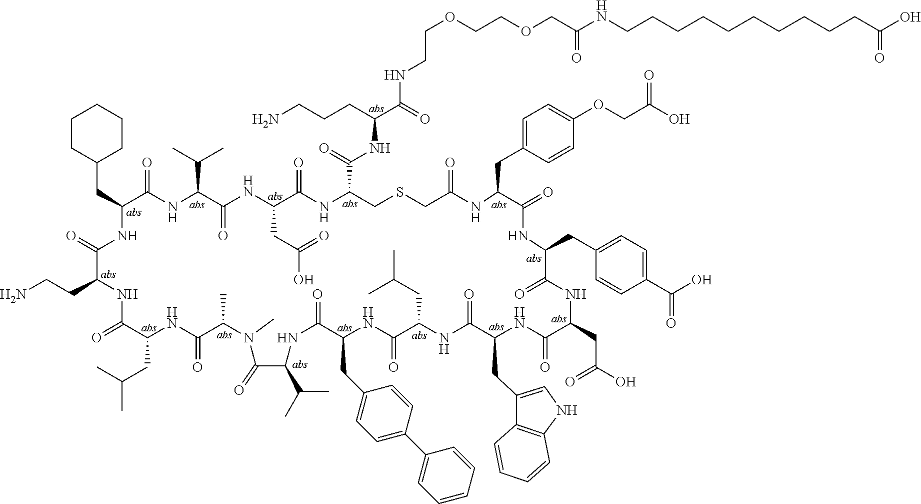 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2125 was prepared on a 50 μmol scale. The yield of the product was 66.8 mg, and its estimated purity by LCMS analysis was 100%. Analysis condition A: Retention time=1.62 min; ESI-MS(+) m/z [M+2H]²⁺: 1210.1.

### Preparation of Example 2126

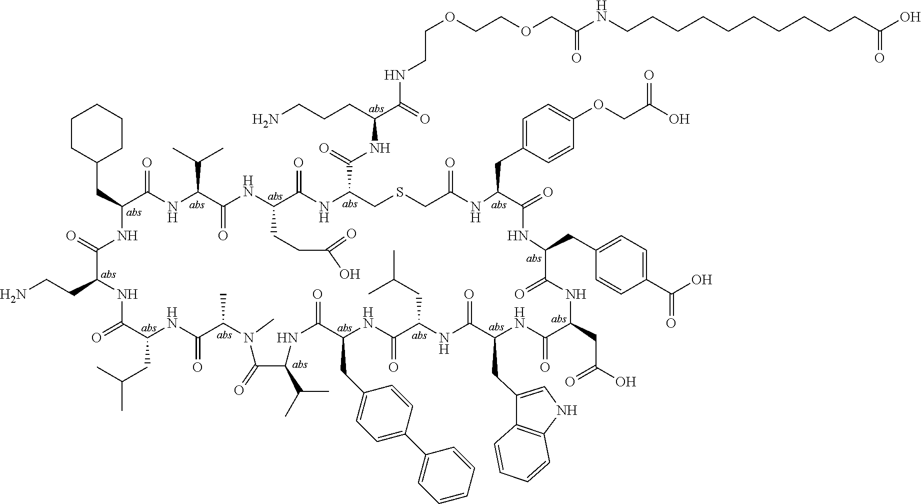 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2126 was prepared on a 50 μmol scale. The yield of the product was 34.1 mg, and its estimated purity by LCMS analysis was 98%. Analysis condition B: Retention time=1.79 min; ESI-MS(+) m/z [M+2H]²⁺: 1217.1.

### Preparation of Example 2127

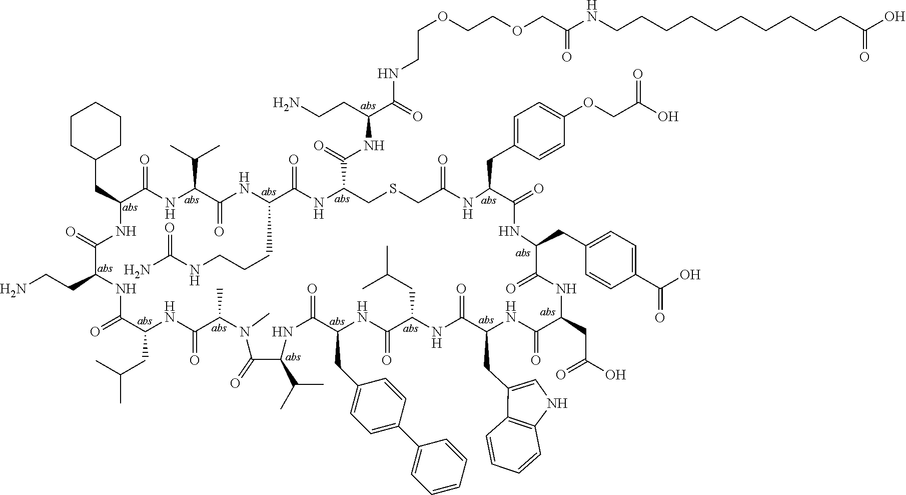 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2127 was prepared on a 50 μmol scale. The yield of the product was 24.8 mg, and its estimated purity by L CMS analysis was 100%. Analysis condition A: Retention time=1.54 min; ESI-MS(+) m/z [M+2H]²⁺: 1224.2.

### Preparation of Example 2128

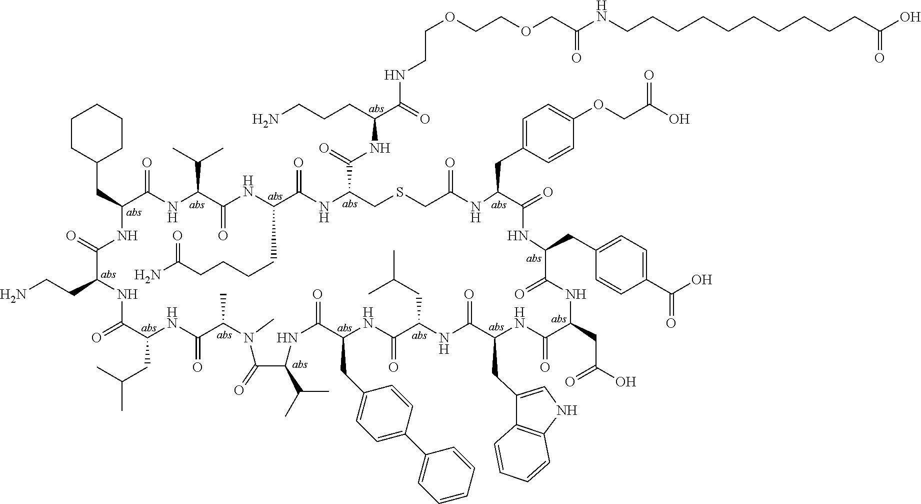 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2128 was prepared on a 50 μmol scale. The yield of the product was 15.1 mg, and its estimated purity by LCMS analysis was 100%. Analysis condition A: Retention time=1.61 min; ESI-MS(+) m/z [M+2H]²⁺: 1231.2.

### Preparation of Example 2129

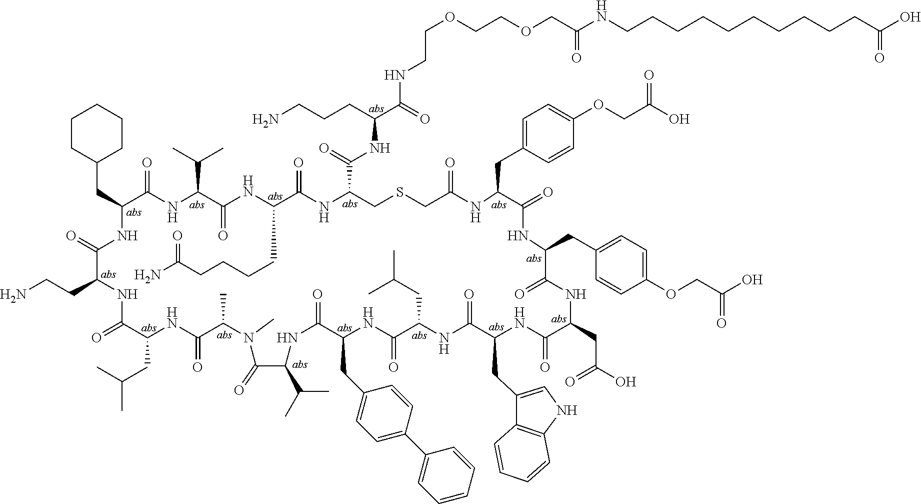 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2129 was prepared on a 50 μmol scale. The yield of the product was 16.3 mg, and its estimated purity by LCMS analysis was 96.8%. Analysis condition B: Retention time=1.77 min; ESI-MS(+) m/z [M+2H]²⁺: 1246.1.

### Preparation of Example 2130

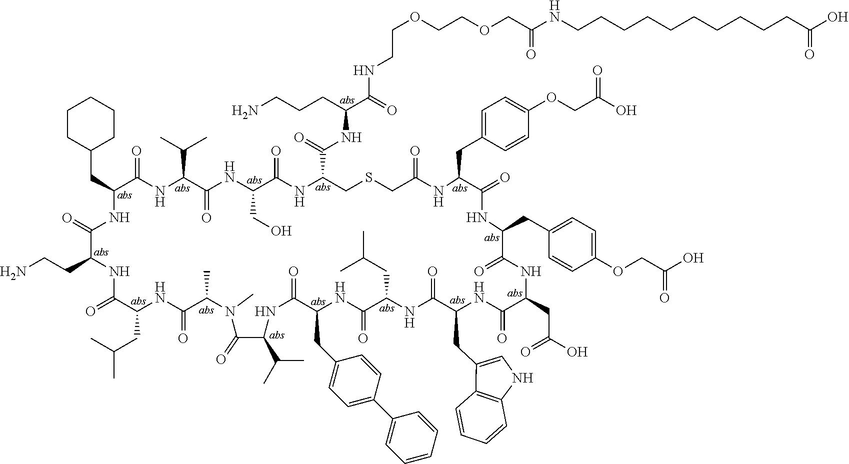 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2130 was prepared on a 50 μmol scale. The yield of the product was 19.2 mg, and its estimated purity by LCMS analysis was 97.4%. Analysis condition B: Retention time=1.77 min; ESI-MS(+) m/z [M+2H]²⁺: 1211.2.

### Preparation of Example 2131

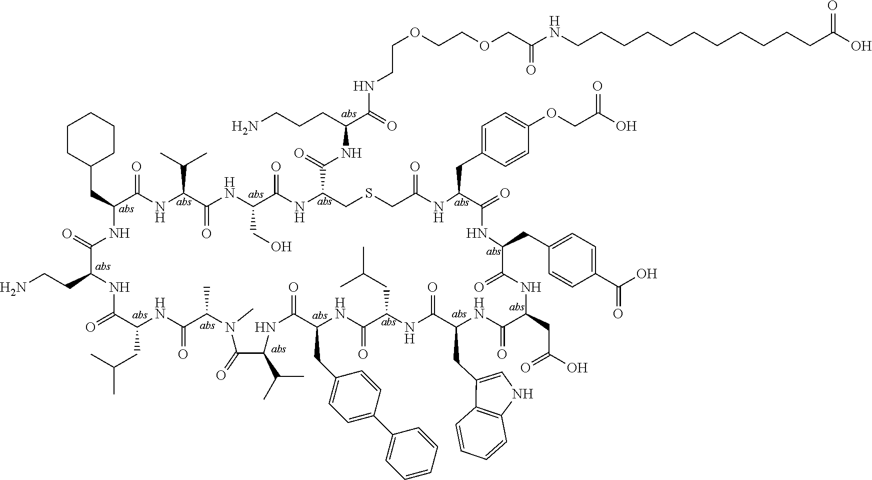 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2131 was prepared on a 50 μmol scale. The yield of the product was 46.9 mg, and its estimated purity by LCMS analysis was 100%. Analysis condition A: Retention time=1.56 min; ESI-MS(+) m/z [M+2H]²⁺: 1203.3.

### Preparation of Example 2132

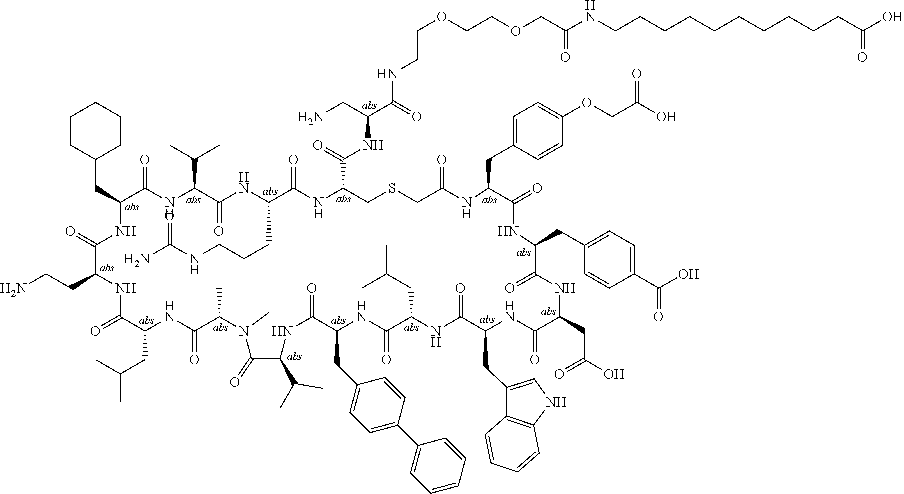 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2132 was prepared on a 50 μmol scale. The yield of the product was 19.3 mg, and its estimated purity by LCMS analysis was 95.7%. Analysis condition B: Retention time=1.79 min; ESI-MS(+) m/z [M+2H]²⁺: 1217.2.

### Preparation of Example 2133

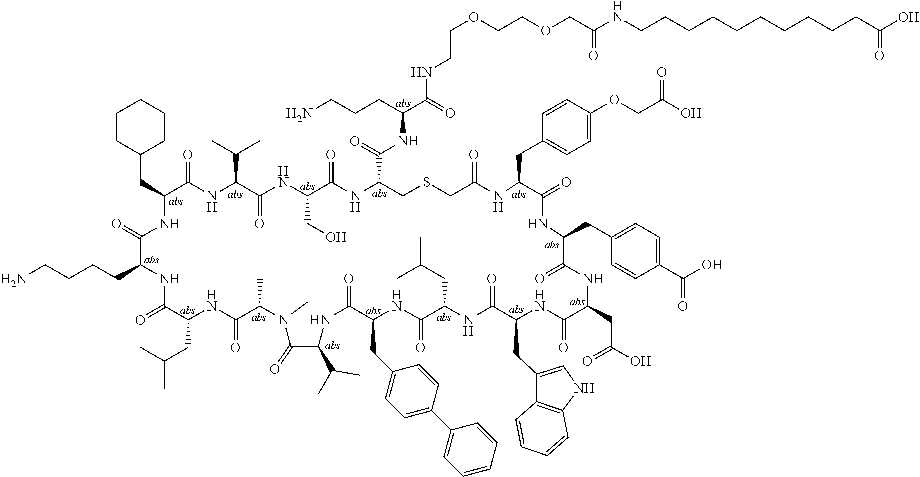 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCCCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2133 was prepared on a 50 μmol scale. The yield of the product was 16.1 mg, and its estimated purity by LCMS analysis was 100%. Analysis condition A: Retention time=1.52 min; ESI-MS(+) m/z [M+2H]²⁺: 1210.2.

### Preparation of Example 2134

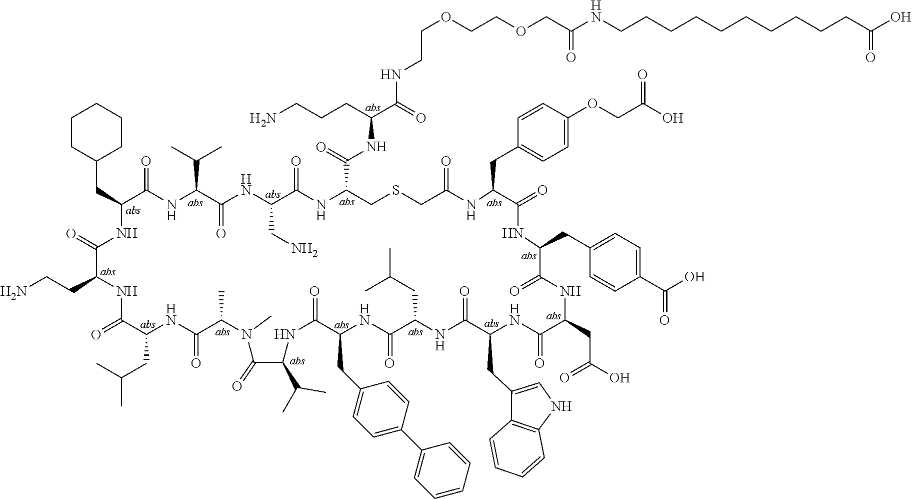 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2134 was prepared on a 50 μmol scale. The yield of the product was 45.7 mg, and its estimated purity by LCMS analysis was 97.7%. Analysis condition B: Retention time=1.73 min; ESI-MS(+) m/z [M+3H]³⁺: 797.2.

### Preparation of Example 2135

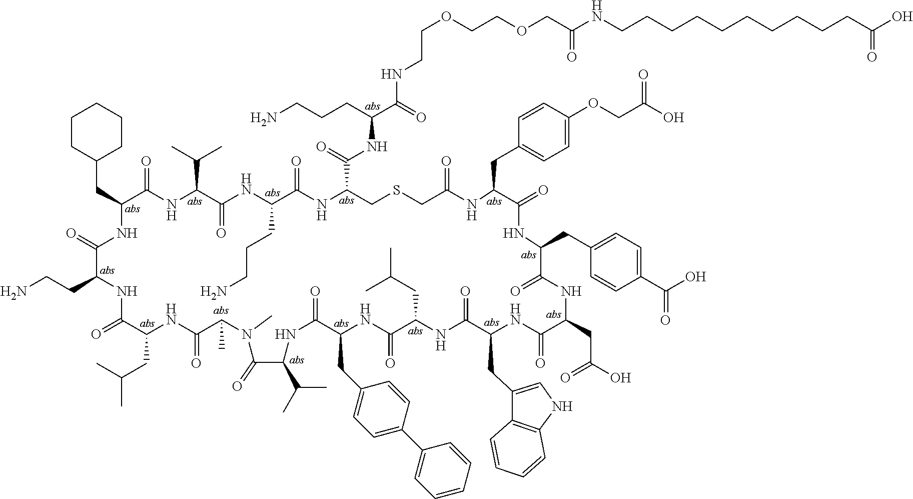 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2135 was prepared on a 50 μmol scale. The yield of the product was 19.5 mg, and its estimated purity by LCMS analysis was 95.4%. Analysis condition B: Retention time=1.8 min; ESI-MS(+) m/z [M+2H]²⁺: 1209.3.

### Preparation of Example 2136

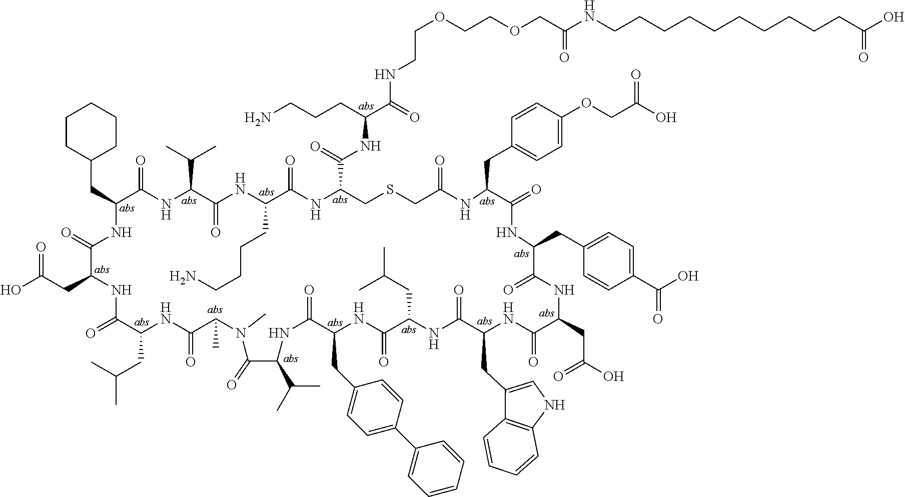 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2136 was prepared on a 50 μmol scale. The yield of the product was 8.6 mg, and its estimated purity by LCMS analysis was 97.4%. Analysis condition B: Retention time=2.04 min; ESI-MS(+) m/z [M+2H]²⁺: 1224.1.

### Preparation of Example 2137

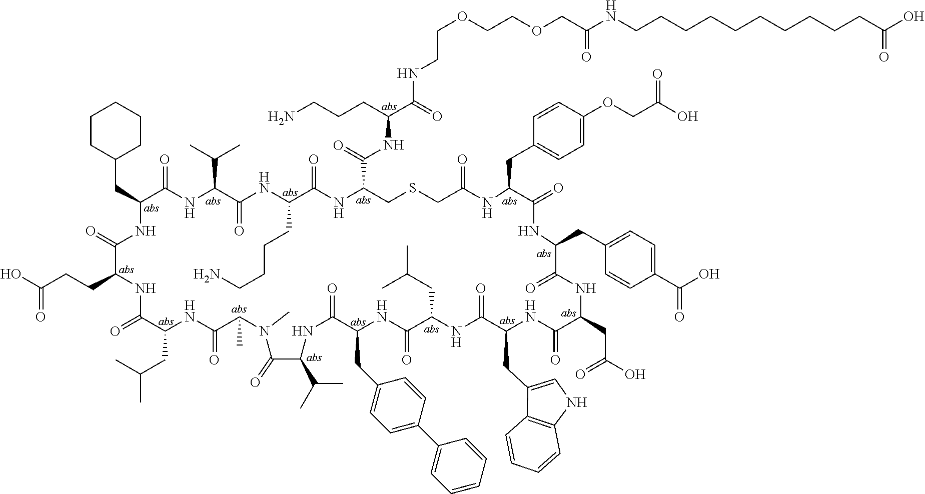 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2137 was prepared on a 50 μmol scale. The yield of the product was 14.4 mg, and its estimated purity by LCMS analysis was 99.1%. Analysis condition A: Retention time=1.5 min; ESI-MS(+) m/z [M+2H]²⁺: 1231.

### Preparation of Example 2138

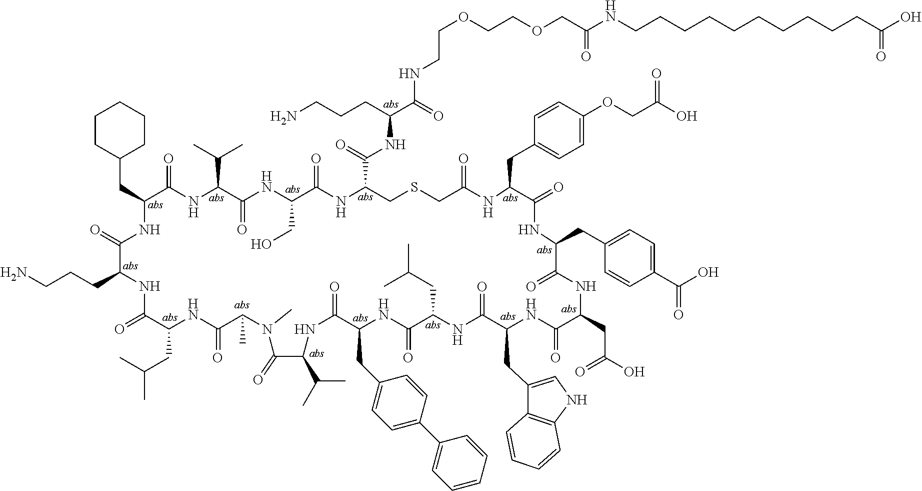 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2138 was prepared on a 50 μmol scale. The yield of the product was 32.4 mg, and its estimated purity by LCMS analysis was 97%. Analysis condition A: Retention time=1.6 min; ESI-MS(+) m/z [M+2H]²⁺: 1203.1.

### Preparation of Example 2139

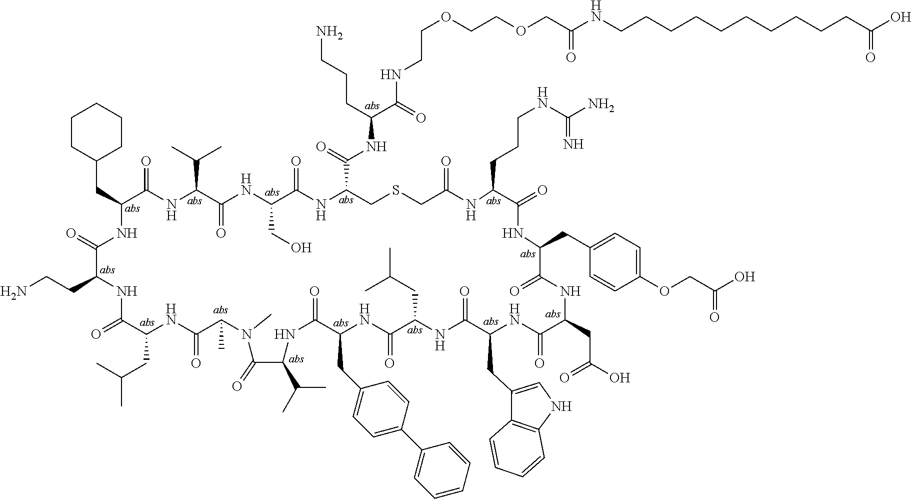 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2139 was prepared on a 50 μmol scale. The yield of the product was 12.5 mg, and its estimated purity by LCMS analysis was 100%. Analysis condition A: Retention time=1.68 min; ESI-MS(+) m/z [M+2H]²⁺: 786.1.

### Preparation of Example 2140

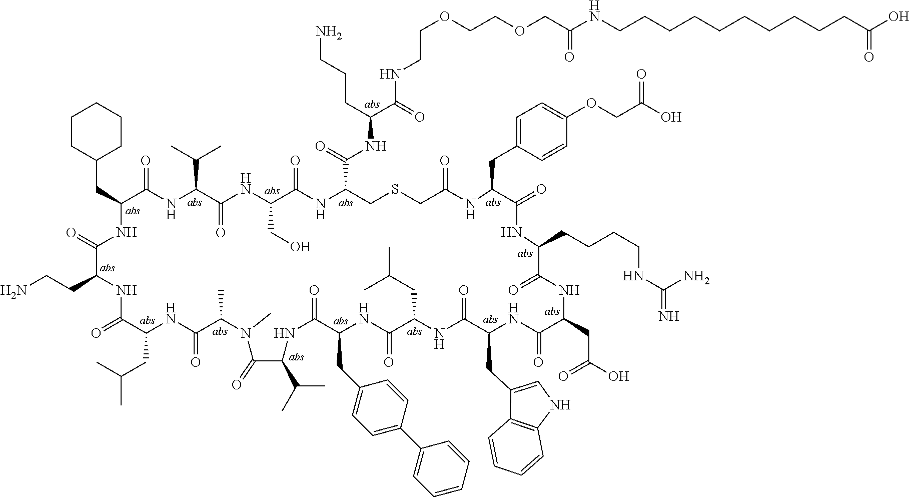 [SMILES: CC(C)C[C@@H]1NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2140 was prepared on a 50 μmol scale. The yield of the product was 12 mg, and its estimated purity by LCMS analysis was 99.3%. Analysis condition B: Retention time=1.65 min; ESI-MS(+) m/z [M+3H]³⁺: 791.2.

### Preparation of Example 2141

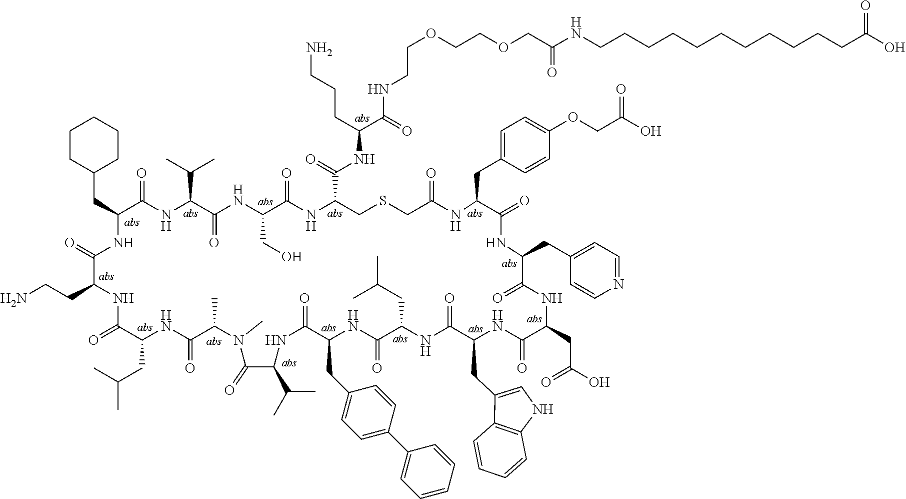 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2141 was prepared on a 50 μmol scale. The yield of the product was 26.8 mg, and its estimated purity by LCMS analysis was 98.6%. Analysis condition B: Retention time=1.79 min; ESI-MS(+) m/z [M+3H]³⁺: 788.

### Preparation of Example 2142

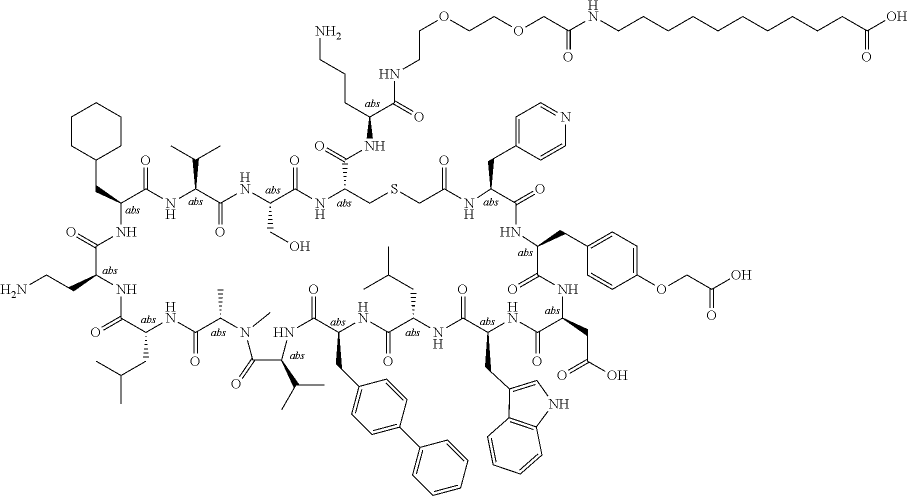 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2142 was prepared on a 50 μmol scale. The yield of the product was 18.7 mg, and its estimated purity by LCMS analysis was 100%. Analysis condition A: Retention time=1.6 min; ESI-MS(+) m/z [M+2H]²⁺: 1175.1.

### Preparation of Example 2143

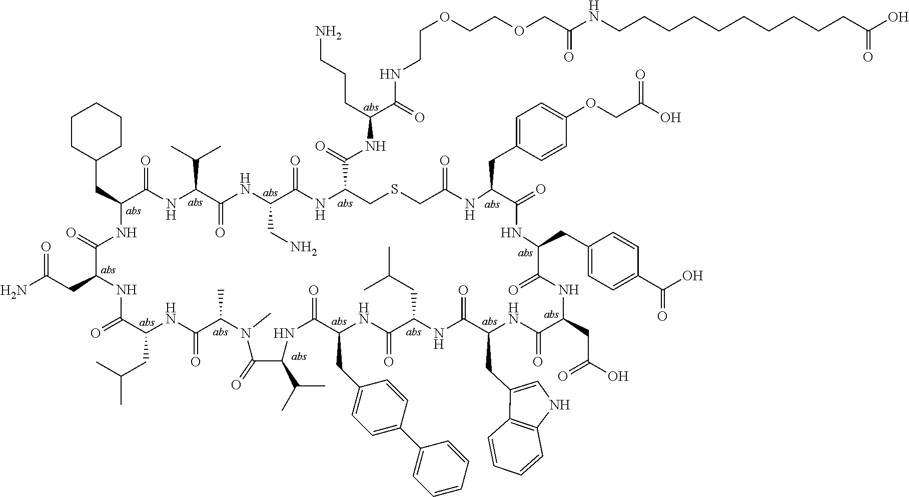 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2143 was prepared on a 50 μmol scale. The yield of the product was 33.8 mg, and its estimated purity by LCMS analysis was 97.2%. Analysis condition A: Retention time=1.74 min; ESI-MS(+) m/z [M+2H]²⁺: 1203.3.

### Preparation of Example 2144

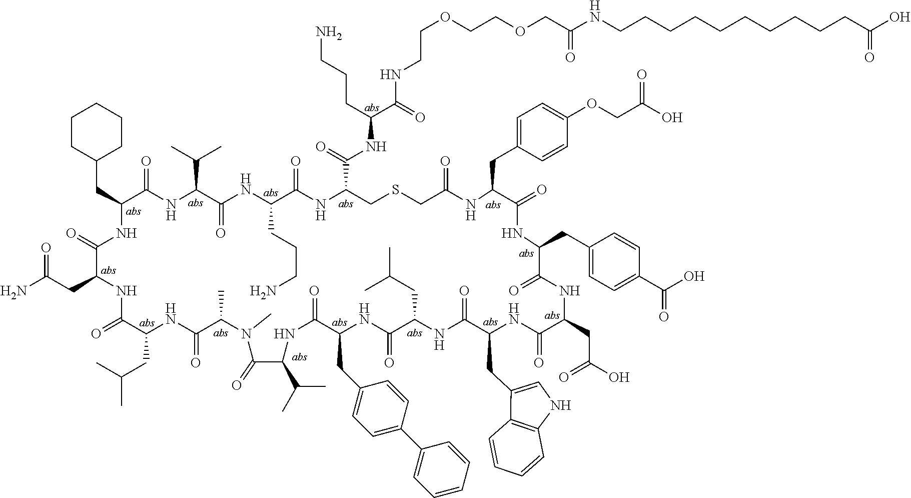 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2144 was prepared on a 50 μmol scale. The yield of the product was 30.6 mg, and its estimated purity by LCMS analysis was 100%. Analysis condition A: Retention time=1.73 min; ESI-MS(+) m/z [M+2H]²⁺: 1217.3.

### Preparation of Example 2145

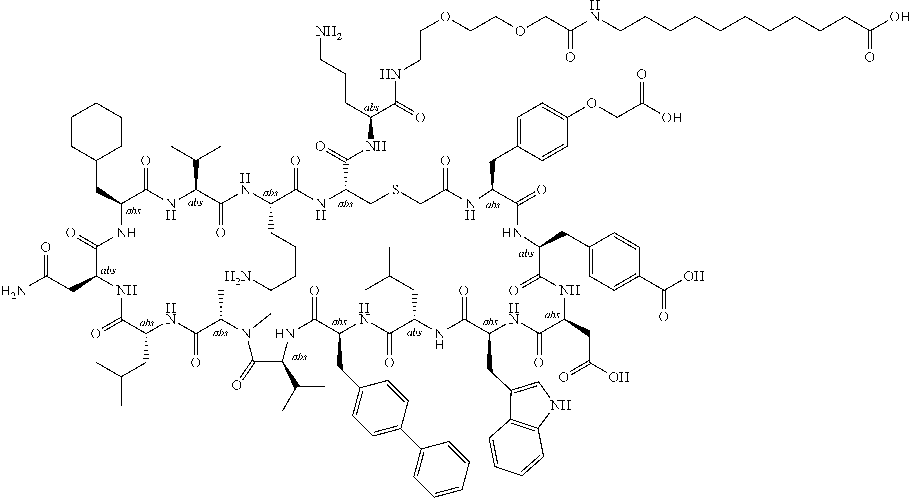 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2145 was prepared on a 50 μmol scale. The yield of the product was 23.1 mg, and its estimated purity by LCMS analysis was 92.1%. Analysis condition A: Retention time=1.75 min; ESI-MS(+) m/z [M+2H]²⁺: 1224.

### Preparation of Example 2146

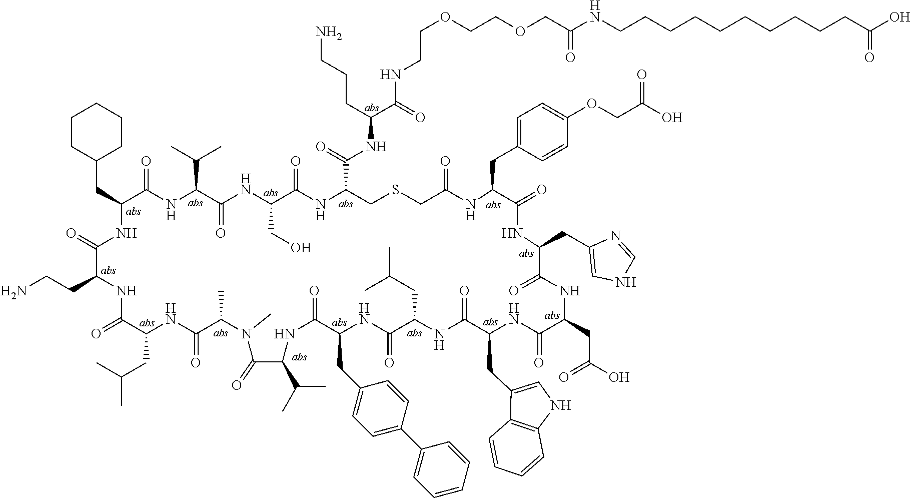 [SMILES: CC(C)C[C@@H]1NC(=O)[C@H](Cc2c[nH]c3ccccc23)NC(=O)[C@H](CC(=O)O)NC(=O)[C@H](Cc2c[nH]cn2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2146 was prepared on a 50 μmol scale. The yield of the product was 24.8 mg, and its estimated purity by LCMS analysis was 99%. Analysis condition B: Retention time=1.76 min; ESI-MS(+) m/z [M+2H]²⁺: 1169.4.

### Preparation of Example 2147

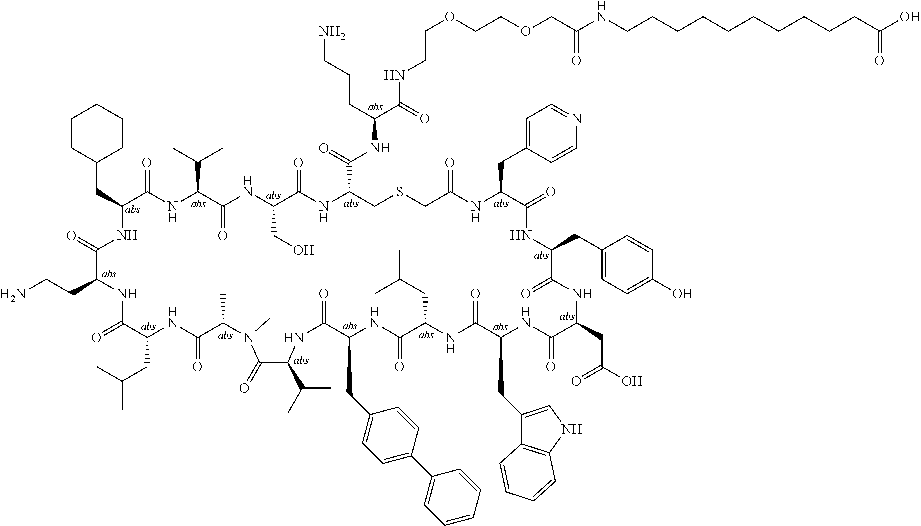 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2147 was prepared on a 50 μmol scale. The yield of the product was 28.9 mg, and its estimated purity by LCMS analysis was 96.1%. Analysis condition B: Retention time=1.75 min; ESI-MS(+) m/z [M+3H]³⁺: 764.1.

### Preparation of Example 2148

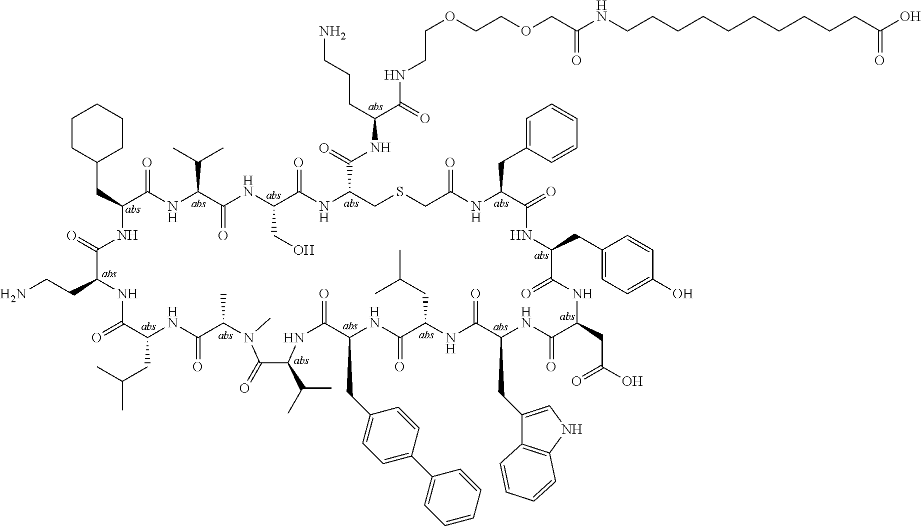 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2148 was prepared on a 50 μmol scale. The yield of the product was 28.6 mg, and its estimated purity by LCMS analysis was 97.8%. Analysis condition B: Retention time=1.97 min; ESI-MS(+) m/z [M+2H]²⁺: 1145.2.

### Preparation of Example 2149

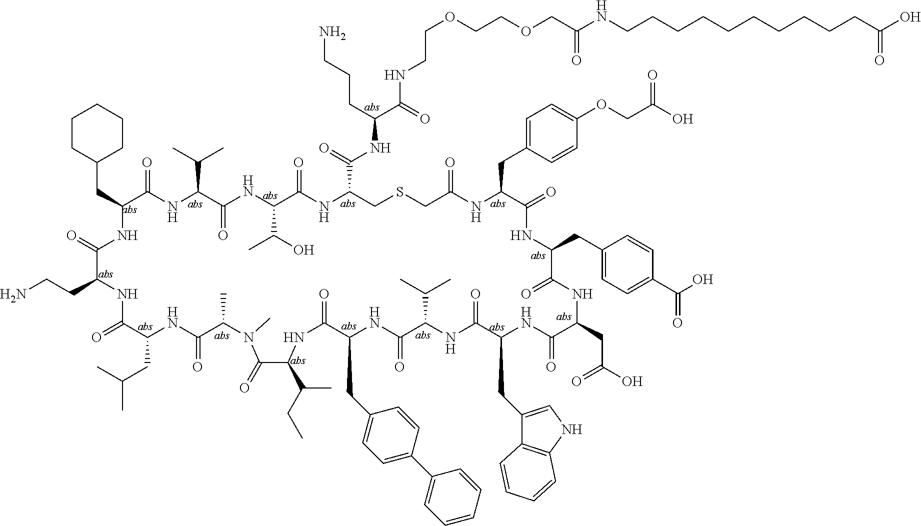 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2149 was prepared on a 50 μmol scale. The yield of the product was 38.9 mg, and its estimated purity by LCMS analysis was 100%. Analysis condition A: Retention time=1.52 min; ESI-MS(+) m/z [M+2H]²⁺: 1203.1.

### Preparation of Example 2150

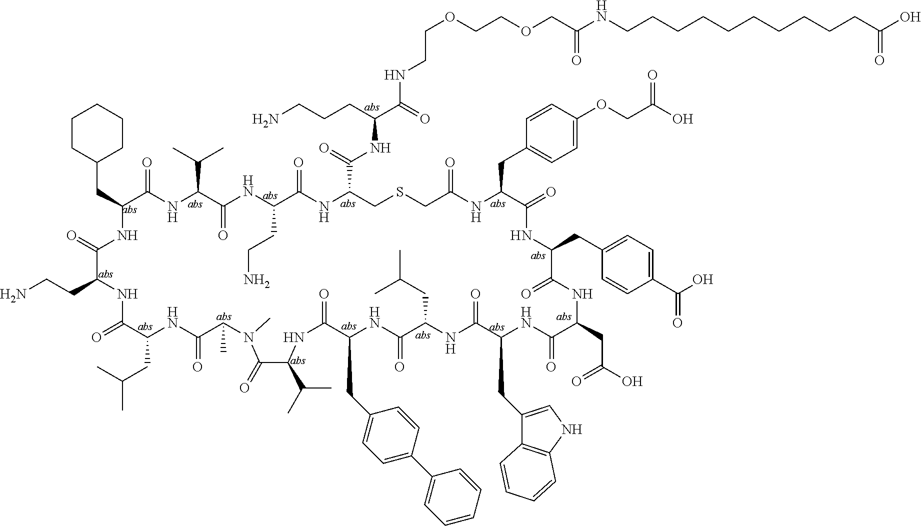 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2150 was prepared on a 50 μmol scale. The yield of the product was 7 mg, and its estimated purity by LCMS analysis was 96.7%. Analysis condition A: Retention time=1.63 min; ESI-MS(+) m/z [M+2H]²⁺: 1203.

### Preparation of Example 2151

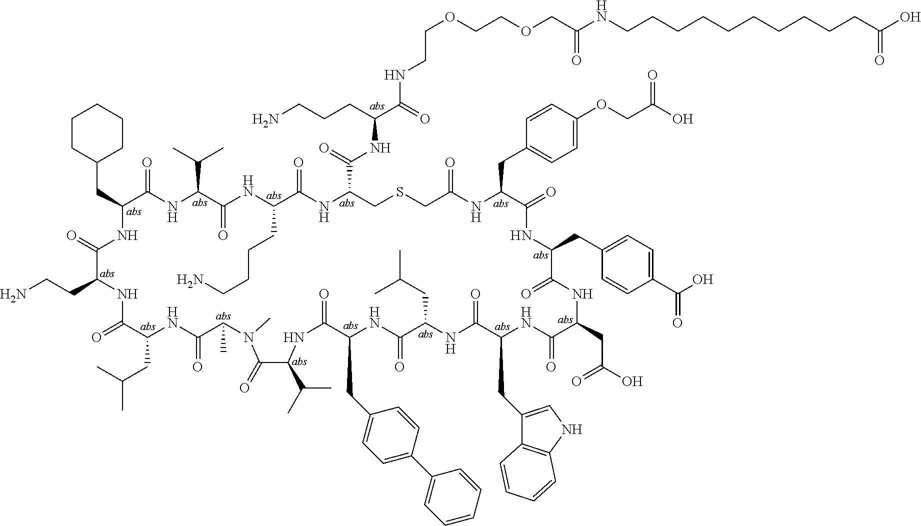 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2151 was prepared on a 50 μmol scale. The yield of the product was 10.1 mg, and its estimated purity by LCMS analysis was 97.9%. Analysis condition B: Retention time=1.78 min; ESI-MS(+) m/z [M+3H]³⁺: 811.2.

### Preparation of Example 2152

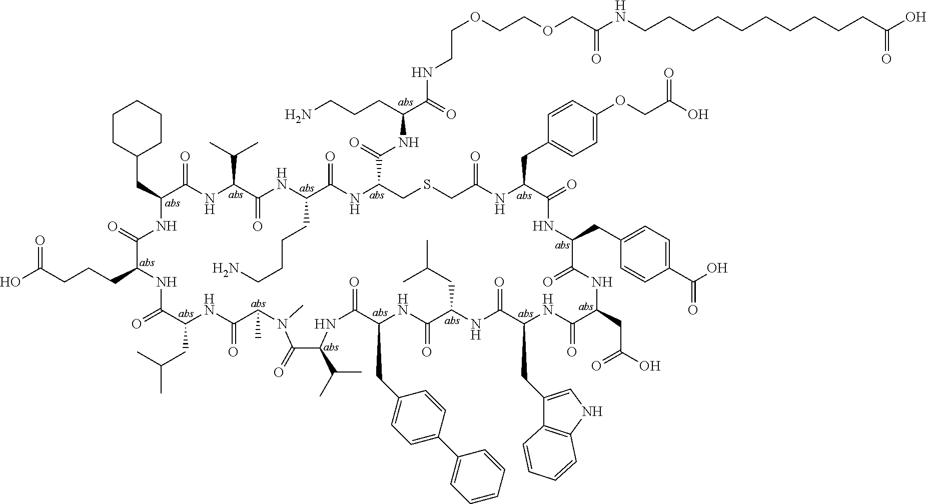 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCCC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2152 was prepared on a 50 μmol scale. The yield of the product was 18.7 mg, and its estimated purity by LCMS analysis was 100%. Analysis condition A: Retention time=1.64 min; ESI-MS(+) m/z [M+2H]²⁺: 1238.2.

### Preparation of Example 2153

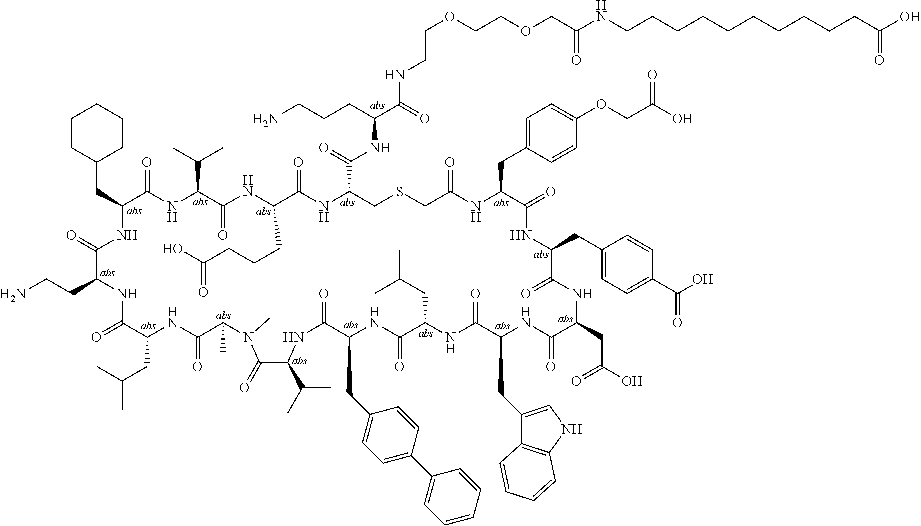 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2153 was prepared on a 50 μmol scale. The yield of the product was 43.6 mg, and its estimated purity by LCMS analysis was 91.1%. Analysis condition B: Retention time=2.17 min; ESI-MS(+) m/z [M+2H]²⁺: 1224.2.

### Preparation of Example 2154

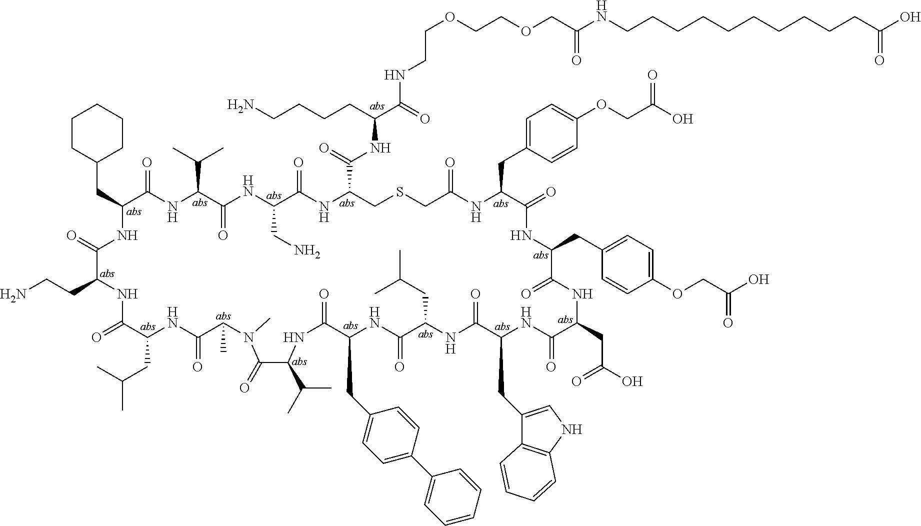 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2154 was prepared on a 50 μmol scale. The yield of the product was 7.8 mg, and its estimated purity by LCMS analysis was 100%. Analysis condition A: Retention time=1.69 min; ESI-MS(+) m/z [M+3H]³⁺: 812.8.

### Preparation of Example 2155

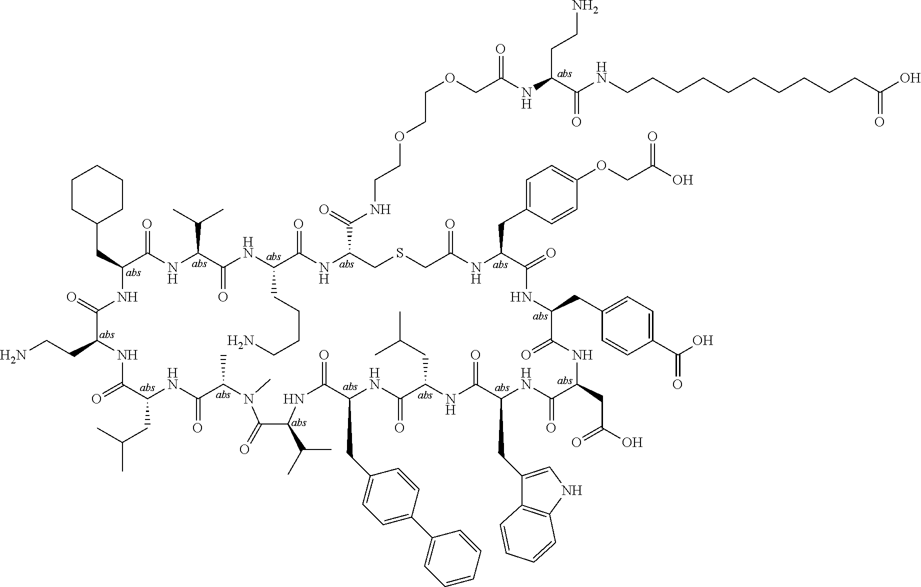 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2155 was prepared on a 50 μmol scale. The yield of the product was 8.2 mg, and its estimated purity by LCMS analysis was 100%. Analysis condition B: Retention time=1.73 min; ESI-MS(+) m/z [M+2H]²⁺: 1210.

### Preparation of Example 2156

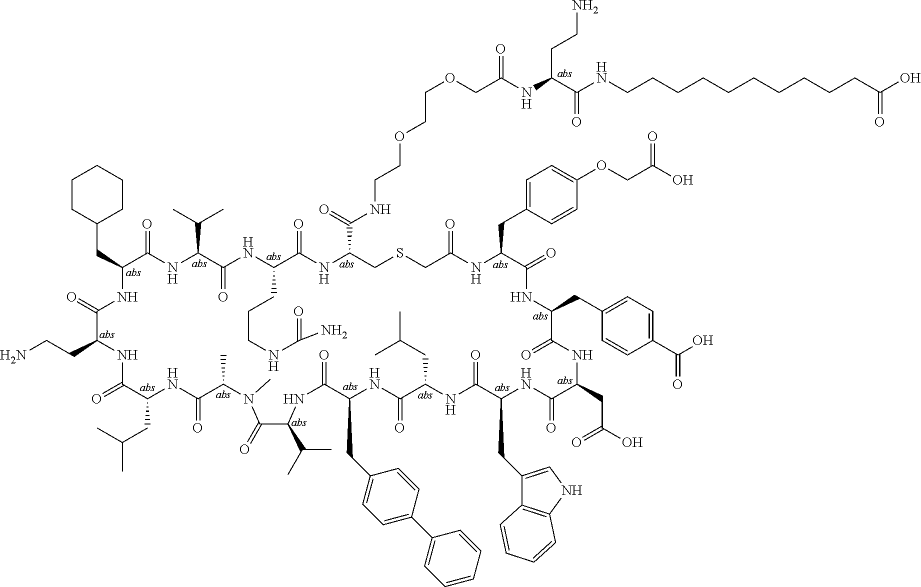 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2156 was prepared on a 50 μmol scale. The yield of the product was 2.1 mg, and its estimated purity by LCMS analysis was 95.2%. Analysis condition B: Retention time=1.67 min; ESI-MS(+) m/z [M+2H]²⁺: 1224.3.

### Preparation of Example 2157

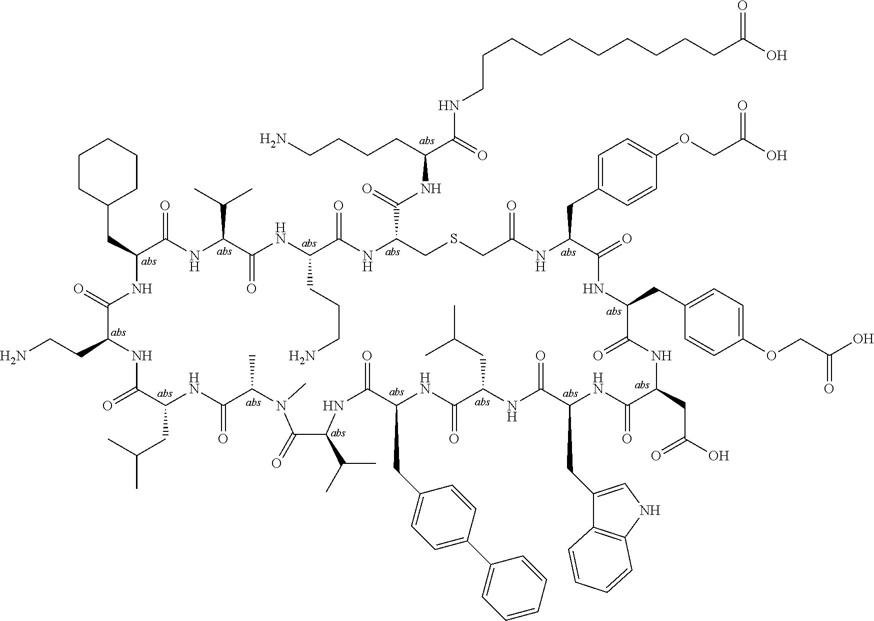 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2157 was prepared on a 50 μmol scale. The yield of the product was 3 mg, and its estimated purity by LCMS analysis was 100%. Analysis condition B: Retention time=1.73 min; ESI-MS(+) m/z [M+2H]²⁺: 1159.1.

### Preparation of Example 2158

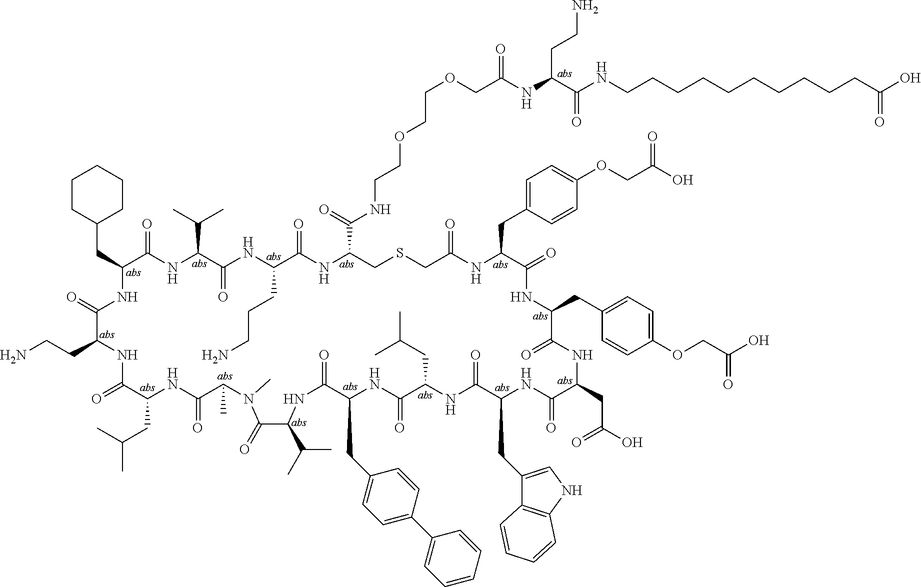 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2158 was prepared on a 50 μmol scale. The yield of the product was 13.2 mg, and its estimated purity by LCMS analysis was 100%. Analysis condition A: Retention time=1.57 min; ESI-MS(+) m/z [M+3H]³⁺: 812.2.

### Preparation of Example 2159

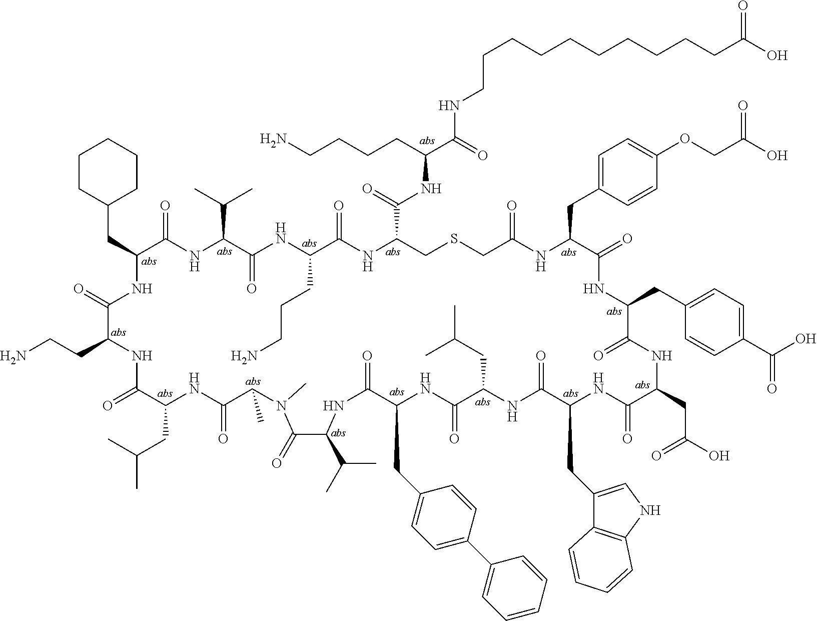 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2159 was prepared on a 50 μmol scale. The yield of the product was 6.2 mg, and its estimated purity by LCMS analysis was 97.300. Analysis condition B: Retention time=1.63 min; ESI-MS(+) m/z [M+2H]²⁺: 1144.1.

### Preparation of Example 2160

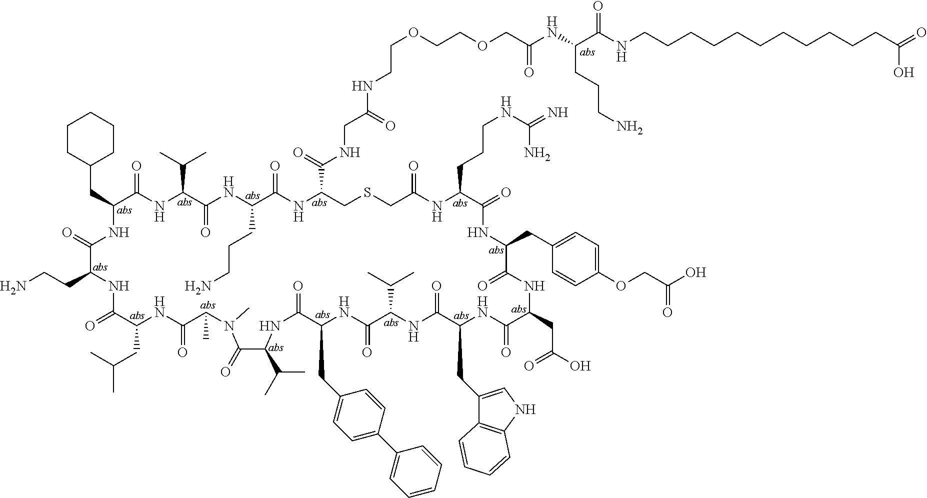 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Example 2160 was prepared on a 50 μmol scale. The yield of the product was 11.9 mg, and its estimated purity by LCMS analysis was 99%. Analysis condition A: Retention time=1.45 min; ESI-MS(+) m/z [M+3H]³⁺: 814.2.

### Preparation of Example 2161

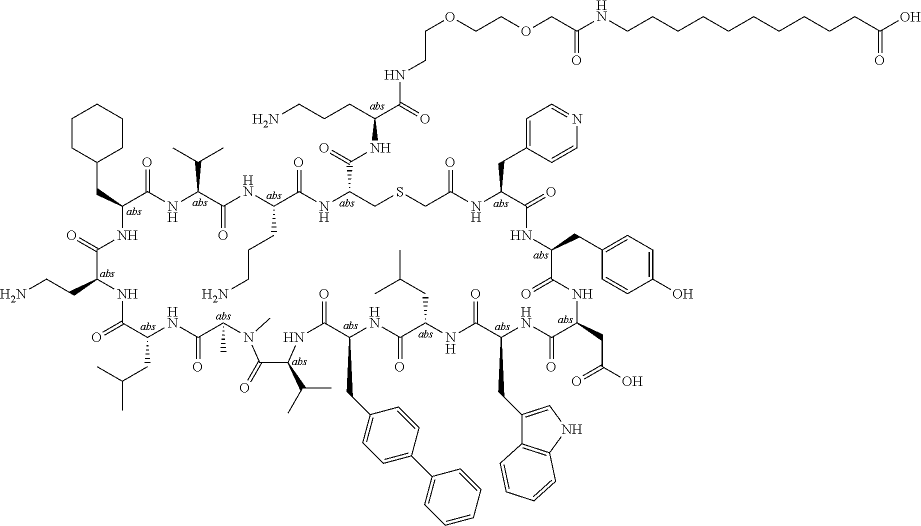 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2161 was prepared on a 50 μmol scale. The yield of the product was 2.2 mg, and its estimated purity by LCMS analysis was 95.7%. Analysis condition B: Retention time=1.73 min; ESI-MS(+) m/z [M+2H]²⁺: 1159.1.

### Preparation of Example 2162

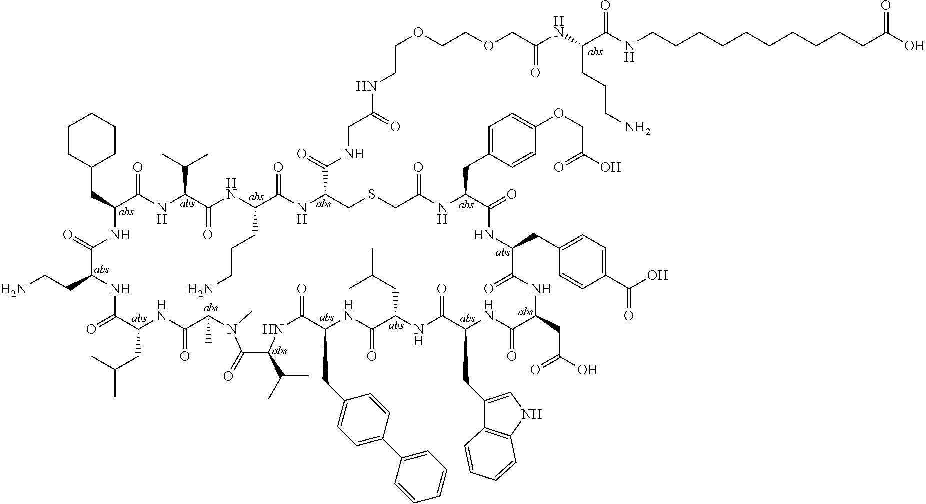 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2162 was prepared on a 50 μmol scale. The yield of the product was 47.6 mg, and its estimated purity by LCMS analysis was 98.5%. Analysis condition A: Retention time=1.51 min; ESI-MS(+) m/z [M+2H]²⁺: 1238.1.

### Preparation of Example 2163

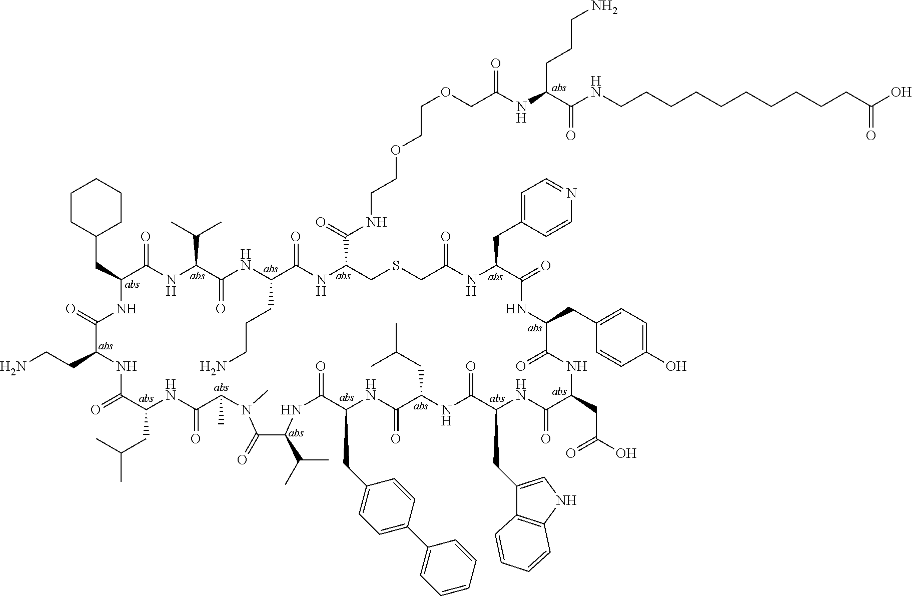 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2163 was prepared on a 50 μmol scale. The yield of the product was 41 mg, and its estimated purity by LCMS analysis was 100%. Analysis condition A: Retention time=1.85 min; ESI-MS(+) m/z [M+2H]²⁺: 1159.2.

### Preparation of Example 2164

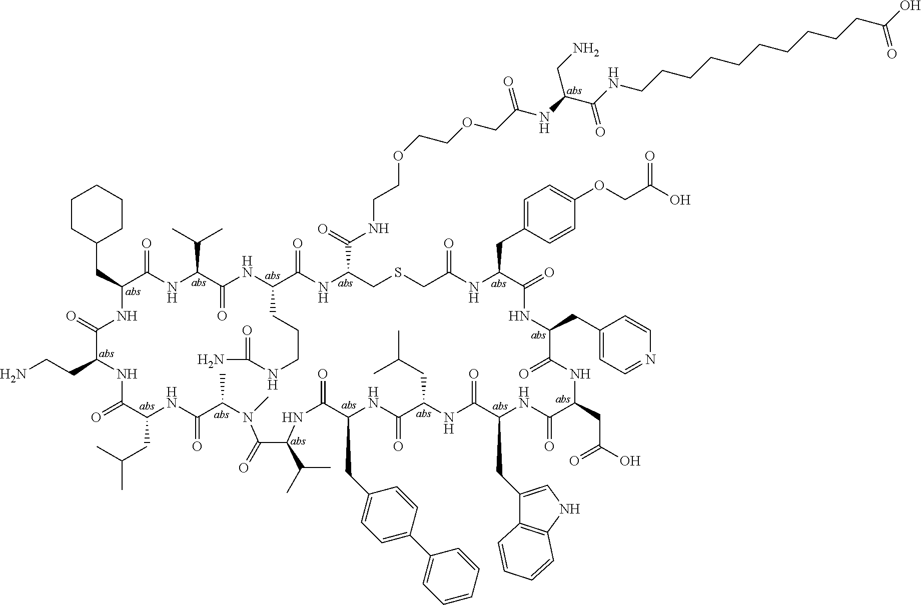 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)N[C@@H](CN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2164 was prepared on a 50 μmol scale. The yield of the product was 6 mg, and its estimated purity by LCMS analysis was 97.6% Analysis condition B Retention time=1.72 min; ESI-MS(+) m/z [M+3H]³⁺: 797.1

### Preparation of Example 2165

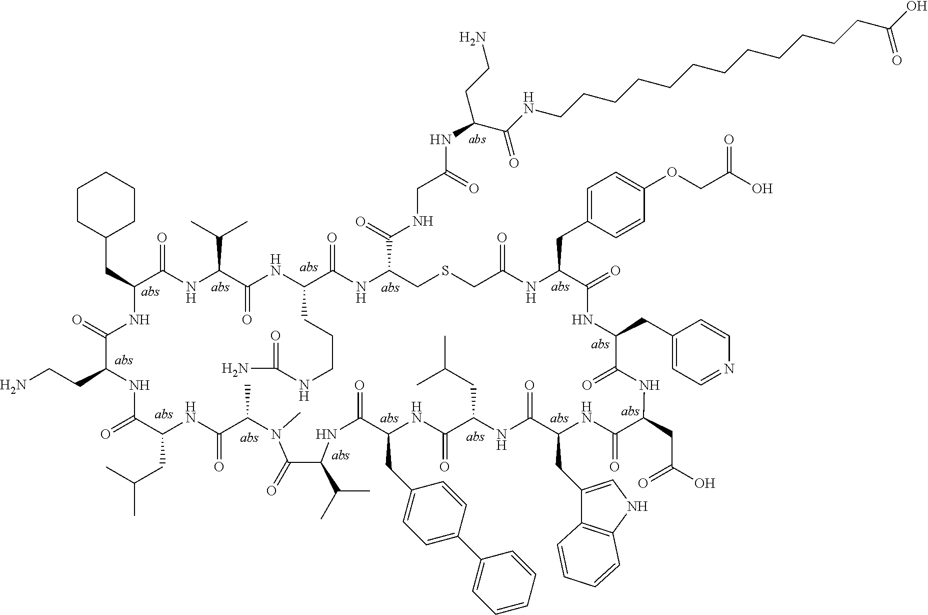 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)N[C@@H](CCN)C(=O)NCCCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2165 was prepared on a 50 μmol scale. The yield of the product was 6.6 mg, and its estimated purity by LCMS analysis was 91.9%. Analysis condition B: Retention time=1.62 min; ESI-MS(+) m/z [M+3H]³⁺: 772.9.

### Preparation of Example 2166

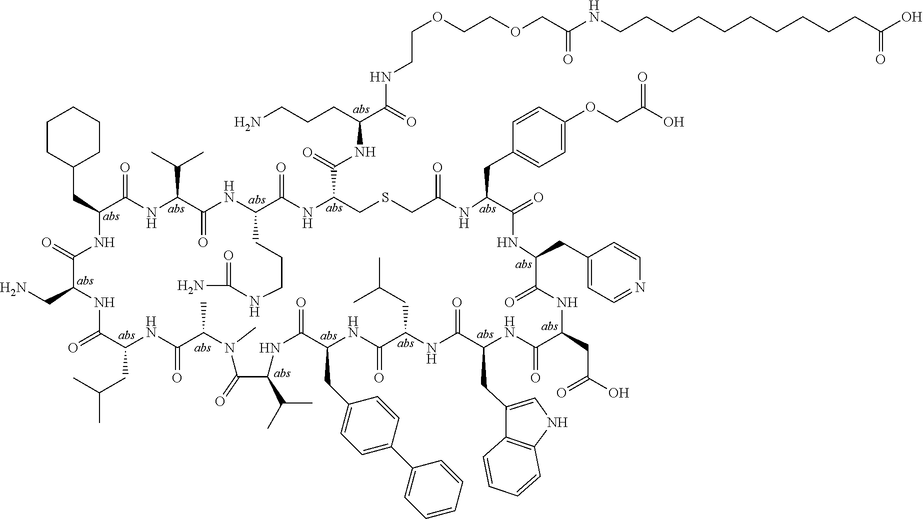 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2166 was prepared on a 50 μmol scale. The yield of the product was 7 mg, and its estimated purity by LCMS analysis was 100%. Analysis condition A: Retention time=1.7 min; ESI-MS(+) m/z [M+3H]³⁺: 802.3.

### Preparation of Example 2167

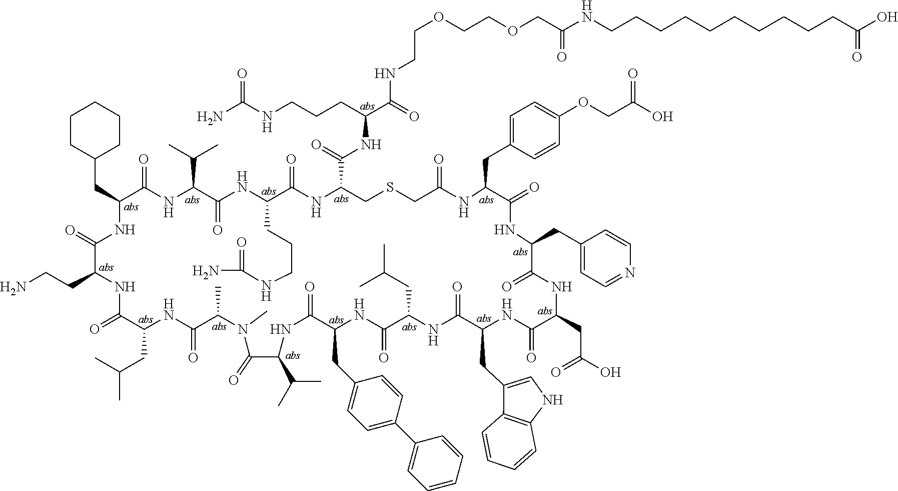 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCNC(N)=O)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2167 was prepared on a 50 μmol scale. The yield of the product was 1.3 mg, and its estimated purity by LCMS analysis was 920%. Analysis condition B: Retention time=1.74 min; ESI-MS(+) m/z [M+3H]⁺: 821.2.

### Preparation of Example 2168

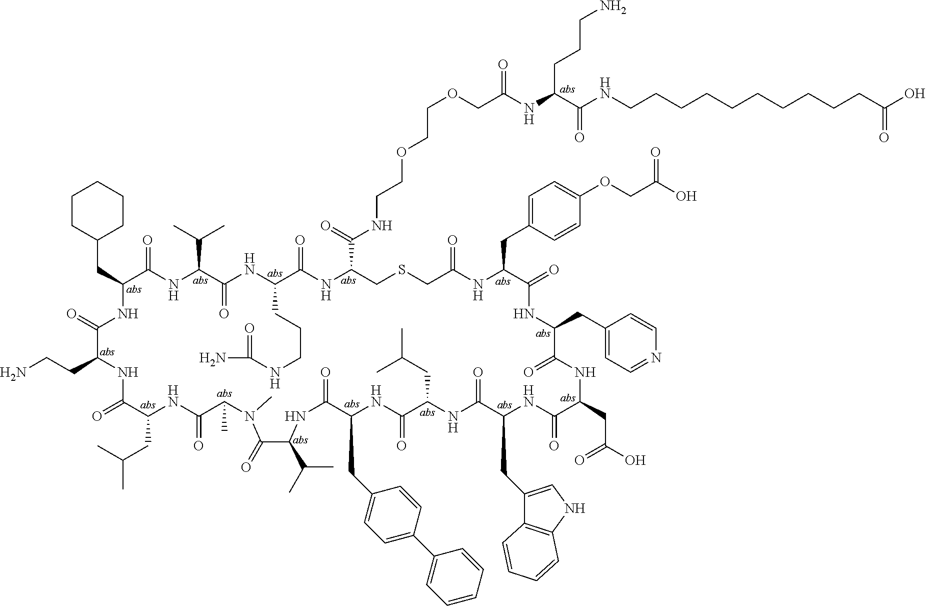 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)N[C@@H](CCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2168 was prepared on a 50 μmol scale. The yield of the product was 4.8 mg, and its estimated purity by LCMS analysis was 98.1%. Analysis condition A: Retention time=1.6 min; ESI-MS(+) m/z [M+3H]³⁺: 807.1.

### Preparation of Example 2169

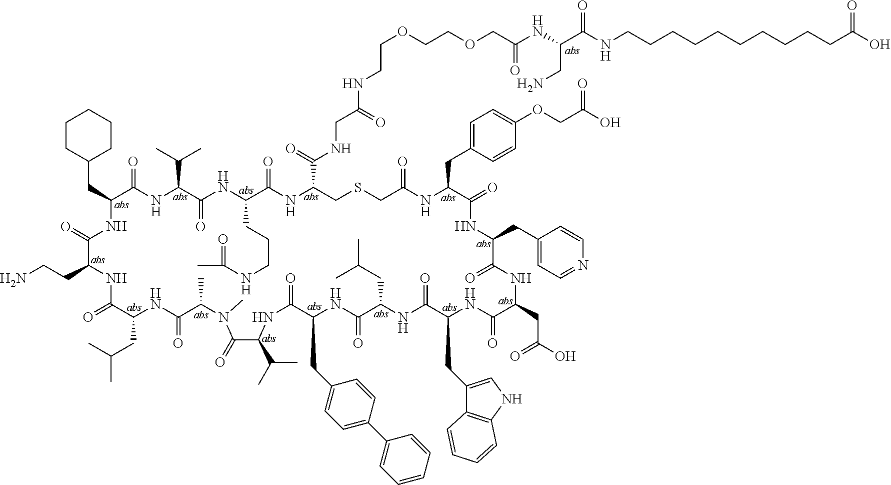 [SMILES: CC(=O)NCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CN)C(=O)NCCCCCCCCCCC(=O)O)NC1=O]

Example 2169 was prepared on a 50 μmol scale. The yield of the product was 4.4 mg, and its estimated purity by LCMS analysis was 99.2%. Analysis condition A: Retention time=1.59 min; ESI-MS(+) m/z [M+2H]²⁺: 1224.1.

### Preparation of Example 2170

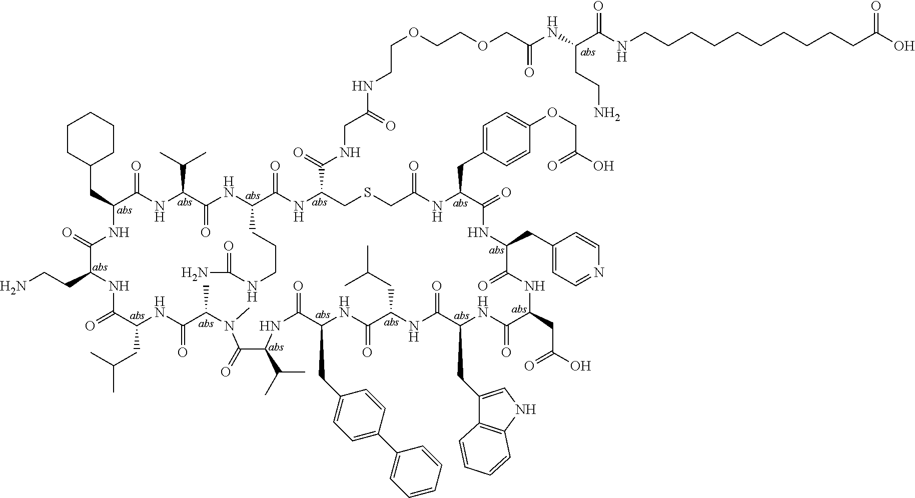 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2170 was prepared on a 50 μmol scale. The yield of the product was 10.5 mg, and its estimated purity by LCMS analysis was 100%. Analysis condition A: Retention time=1.58 min; ESI-MS(+) m/z [M+2H]²⁺: 1231.2.

### Preparation of Example 2171

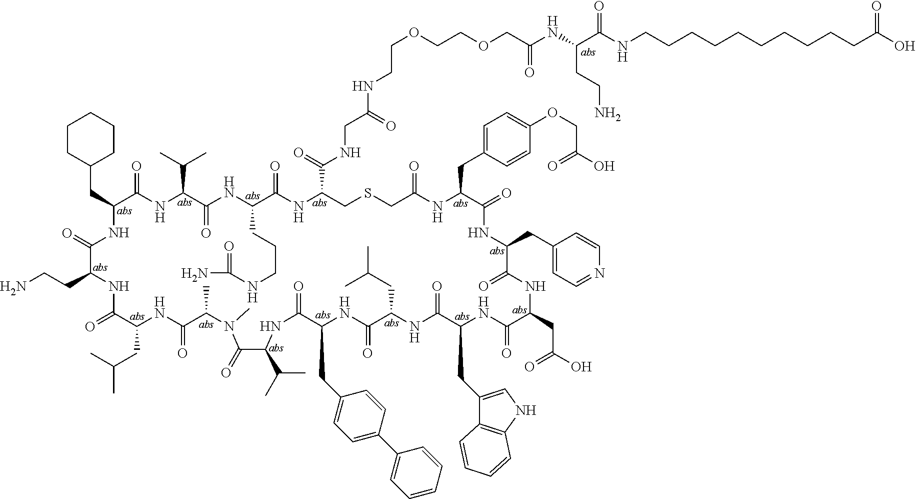 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2171 was prepared on a 50 μmol scale. The yield of the product was 5.4 mg, and its estimated purity by LCMS analysis was 98.5%. Analysis condition A: Retention time=1.69 min; ESI-MS(+) m/z [M+2H]²⁺: 1237.9.

### Preparation of Example 2172

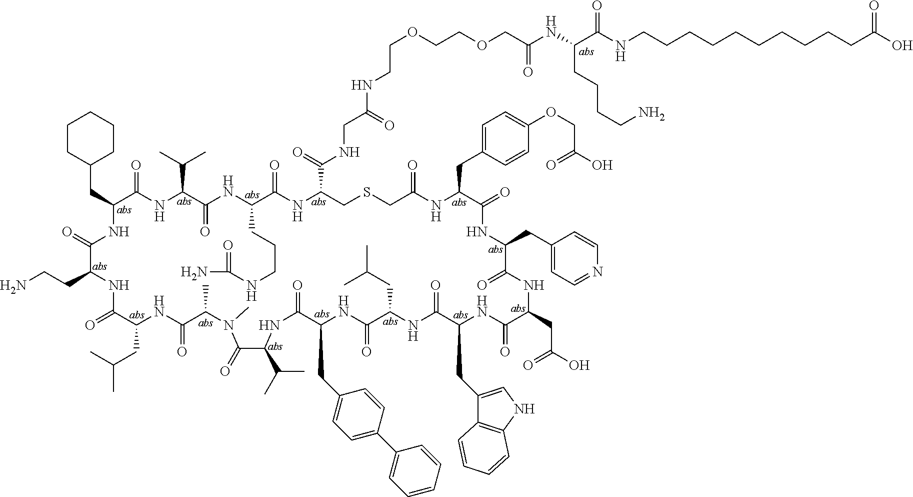 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2172 was prepared on a 50 μmol scale. The yield of the product was 2.5 mg, and its estimated purity by LCMS analysis was 100%. Analysis condition A: Retention time=1.73 min; ESI-MS(+) m/z [M+2H]²⁺: 1245.2.

### Preparation of Example 2173

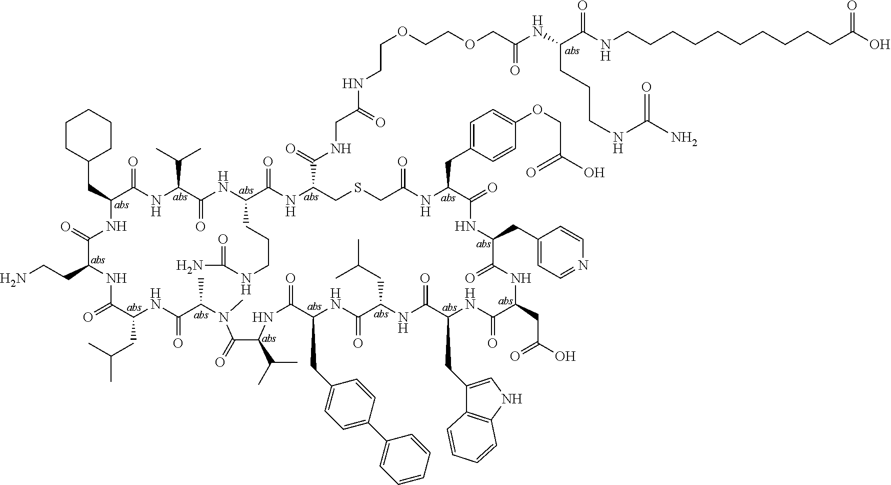 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCCNC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2173 was prepared on a 50 μmol scale. The yield of the product was 9.5 mg, and its estimated purity by LCMS analysis was 93.9%. Analysis condition B: Retention time=1.78 min; ESI-MS(+) m/z [M+3H]³⁺: 840.1.

### Preparation of Example 2174

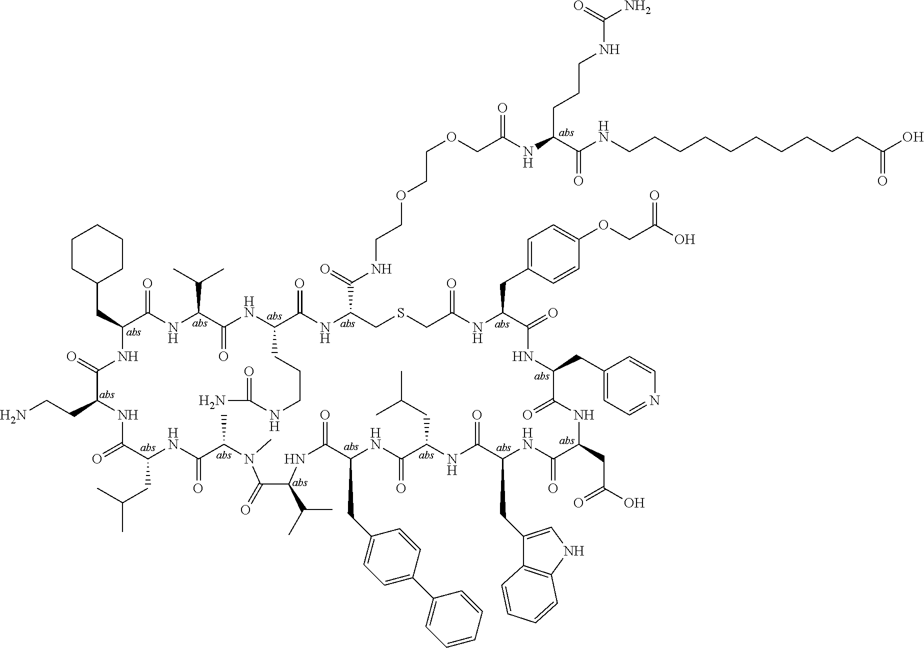 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)N[C@@H](CCCNC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2174 was prepared on a 50 μmol scale. The yield of the product was 6.7 mg, and its estimated purity by LCMS analysis was 94.2%. Analysis condition B: Retention time=1.79 min; ESI-MS(+) m/z [M+2H]²⁺: 1231.3.

### Preparation of Example 2175

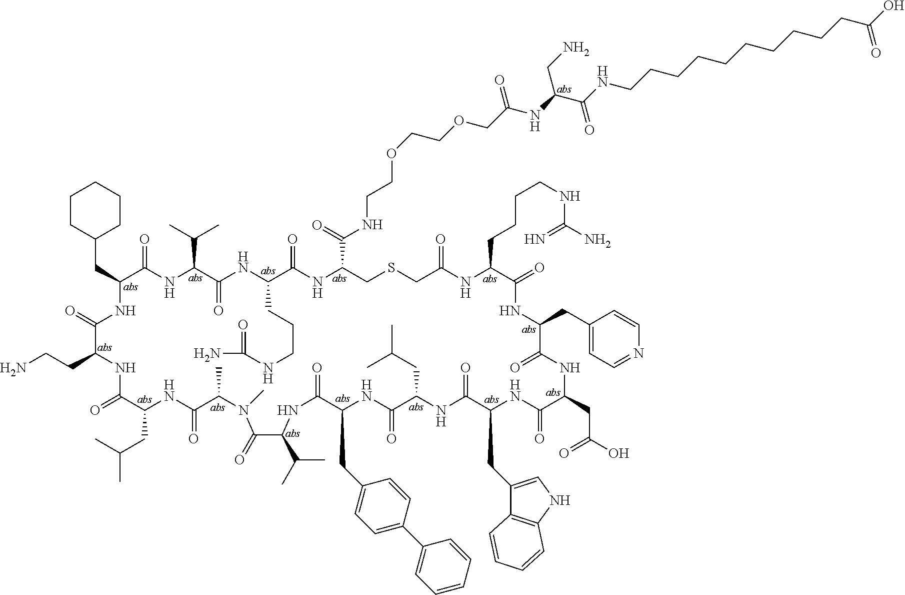 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](CCCCNC(=N)N)NC(=O)CSC[C@@H](C(=O)NCCOCCOCC(=O)N[C@@H](CN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2175 was prepared on a 50 μmol scale. The yield of the product was 2 mg, and its estimated purity by LCMS analysis was 100%. Analysis condition A: Retention time=1.63 min; ESI-MS(+) m/z [M+2H]²⁺: 1206.9.

### Preparation of Example 2176

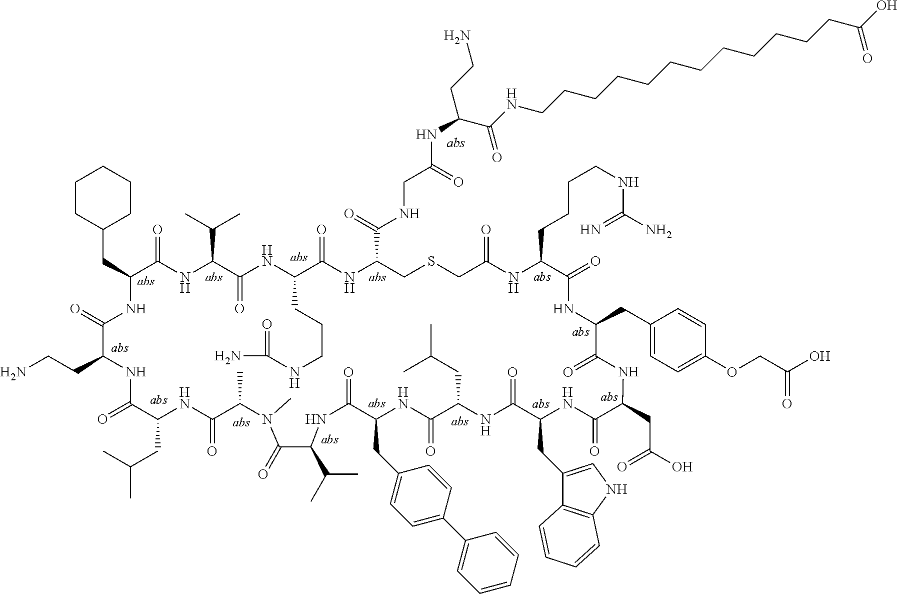 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)N[C@@H](CCN)C(=O)NCCCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2176 was prepared on a 50 μmol scale. The yield of the product was 4.2 mg, and its estimated purity by LCMS analysis was 94.4%. Analysis condition B: Retention time=1.73 min; ESI-MS(+) m/z [M+3H]³⁺: 780.3.

### Preparation of Example 2177

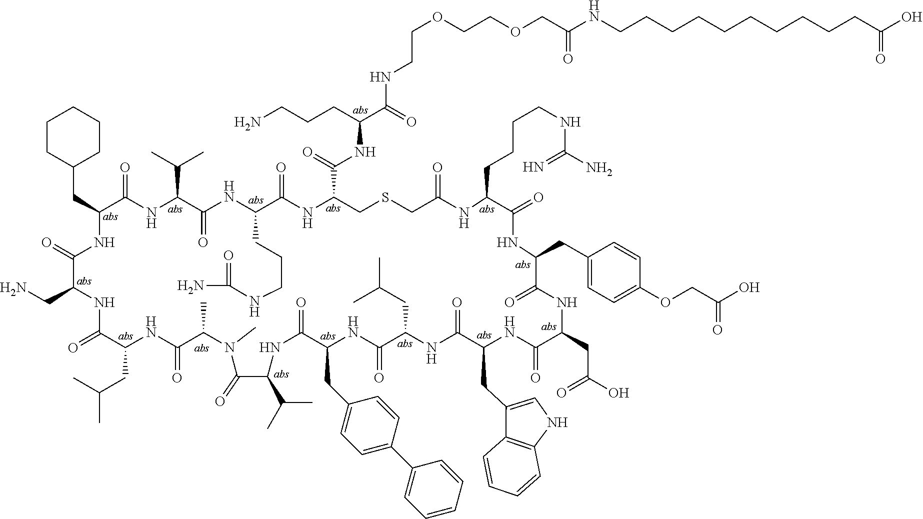 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCN)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2177 was prepared on a 50 μmol scale. The yield of the product was 1.8 mg, and its estimated purity by LCMS analysis was 98.2%. Analysis condition B: Retention time=1.71 min; ESI-MS(+) m/z [M+3H]³⁺: 809.4.

### Preparation of Example 2178

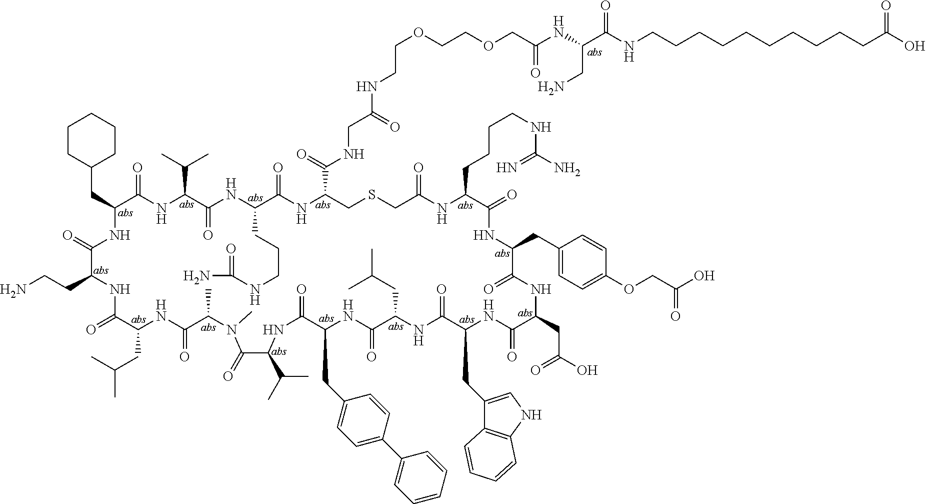 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2178 was prepared on a 50 μmol scale. The yield of the product was 3.5 mg, and its estimated purity by LCMS analysis was 100%. Analysis condition A: Retention time=1.68 min; ESI-MS(+) m/z [M+2H]²⁺: 1235.2.

### Preparation of Example 2179

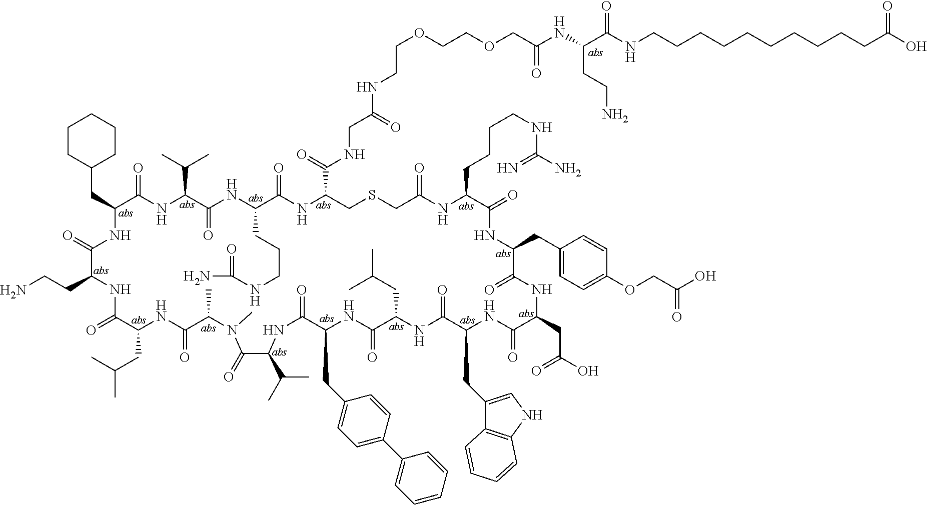 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2179 was prepared on a 50 μmol scale. The yield of the product was 2.3 mg, and its estimated purity by LCMS analysis was 100%. Analysis condition A: Retention time=1.61 min; ESI-MS(+) m/z [M+2H]²⁺: 1242.1.

### Preparation of Example 2180

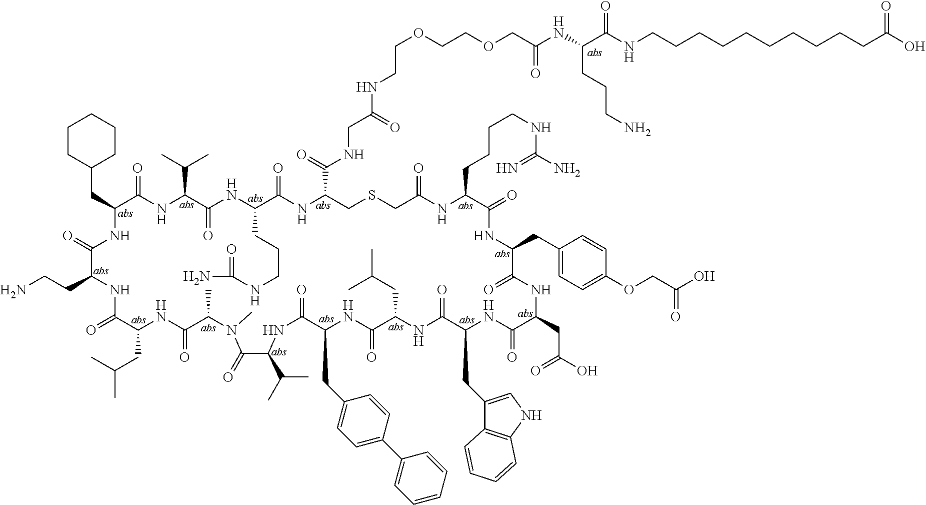 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2180 was prepared on a 50 μmol scale. The yield of the product was 2.7 mg, and its estimated purity by LCMS analysis was 100%. Analysis condition A: Retention time=1.67 min; ESI-MS(+) m/z [M+2H]²⁺: 1249.1.

### Preparation of Example 2181

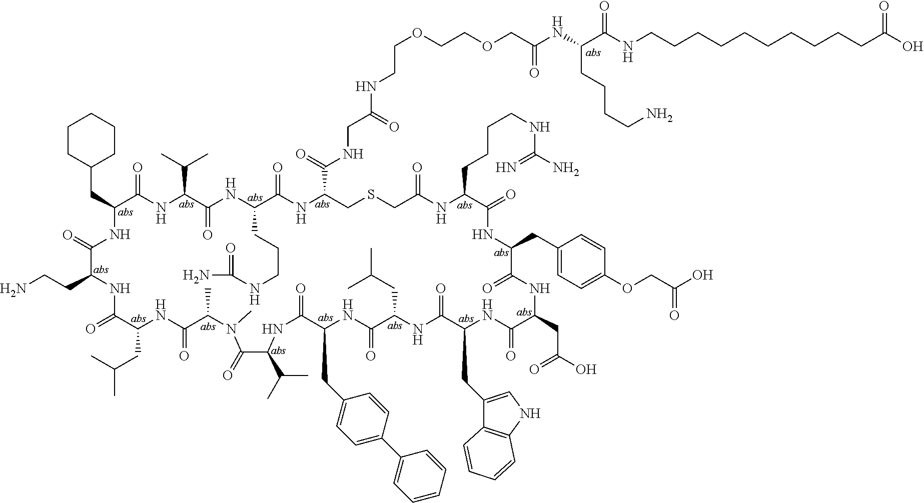 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2181 was prepared on a 50 μmol scale. The yield of the product was 2 mg, and its estimated purity by LCMS analysis was 100%. Analysis condition A: Retention time=1.68 min; ESI-MS(+) m/z [M+2H]²⁺: 1256.2.

### Preparation of Example 2182

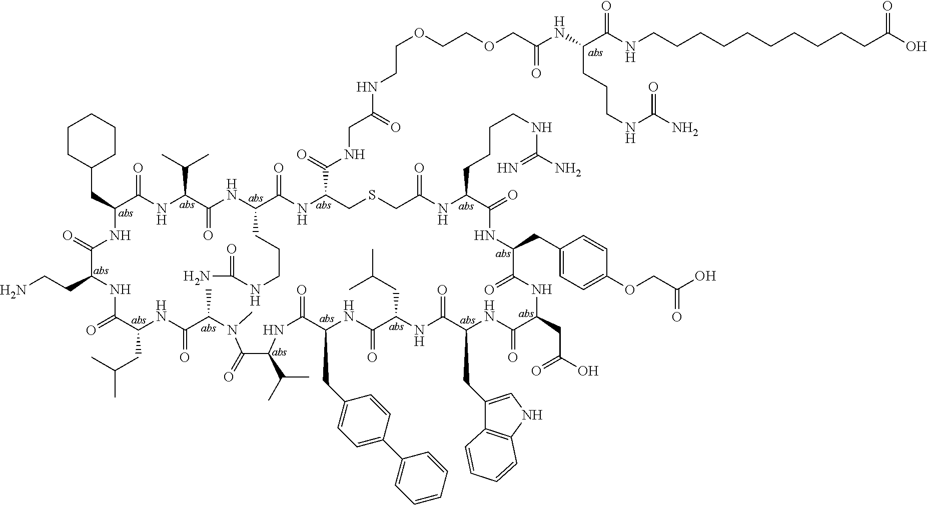 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCNC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2182 was prepared on a 50 μmol scale. The yield of the product was 1.6 mg, and its estimated purity by LCMS analysis was 100%. Analysis condition A: Retention time=1.74 min; ESI-MS(+) m/z [M+3H]³⁺: 847.4.

### Preparation of Example 2183

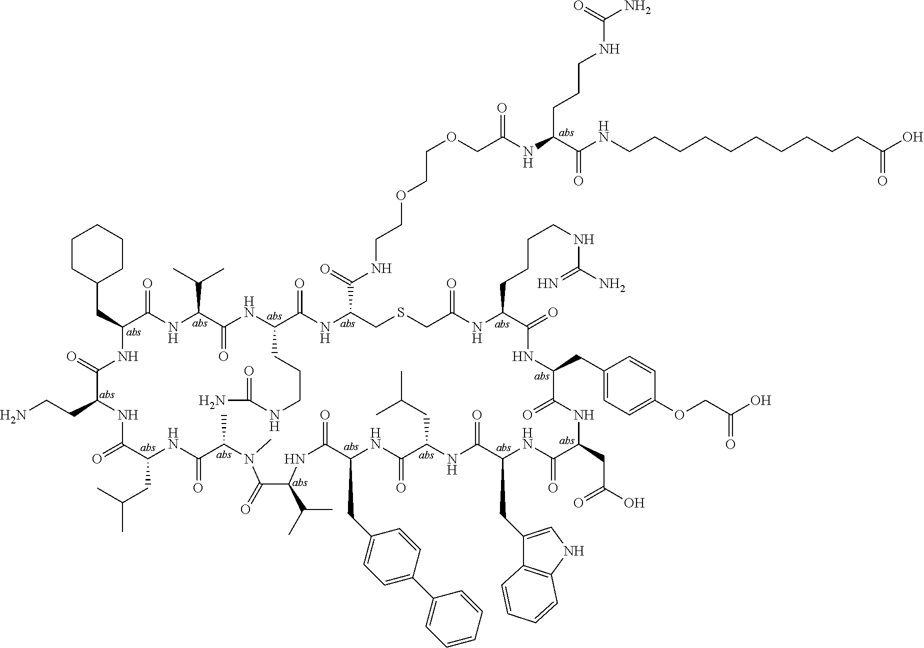 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCNC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2183 was prepared on a 50 μmol scale. The yield of the product was 1.7 mg, and its estimated purity by LCMS analysis was 100%. Analysis condition A: Retention time=1.69 min; ESI-MS(+) m/z [M+2H]²⁺: 1242.6.

### Preparation of Example 2184

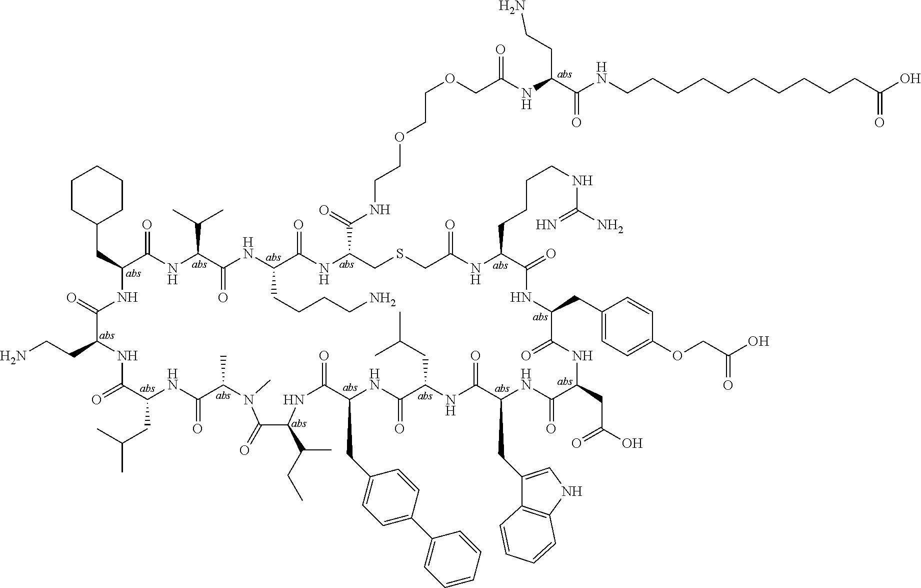 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2184 was prepared on a 50 μmol scale. The yield of the product was 21.7 mg, and its estimated purity by LCMS analysis was 100%. Analysis condition A: Retention time=1.66 min; ESI-MS(+) m/z [M+2H]²⁺: 1206.2.

### Preparation of Example 2185

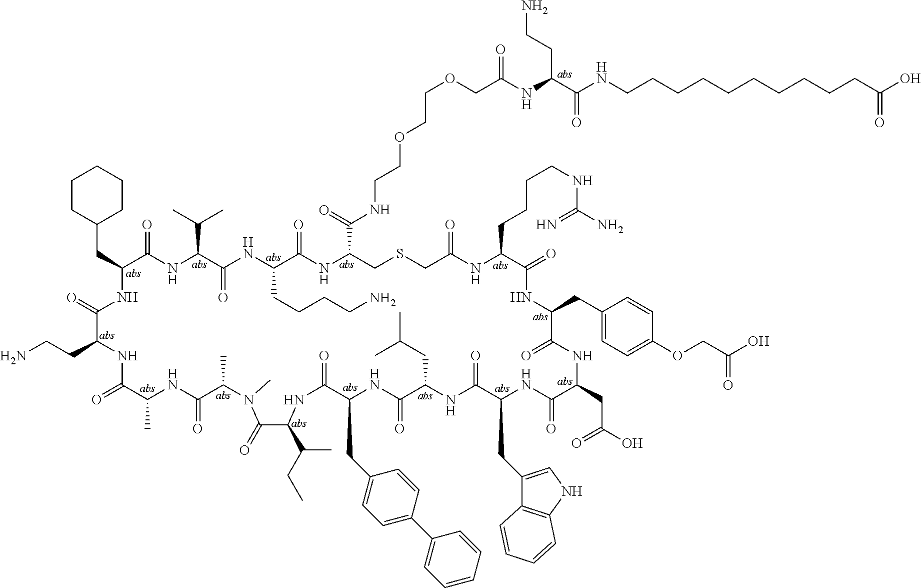 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](C)NC(=O)[C@H](C)N(C)C1=O]

Example 2185 was prepared on a 50 μmol scale. The yield of the product was 21 mg, and its estimated purity by LCMS analysis was 99.4%. Analysis condition A: Retention time=1.48 min; ESI-MS(+) m/z [M+2H]²⁺: 791.1

### Preparation of Example 2186

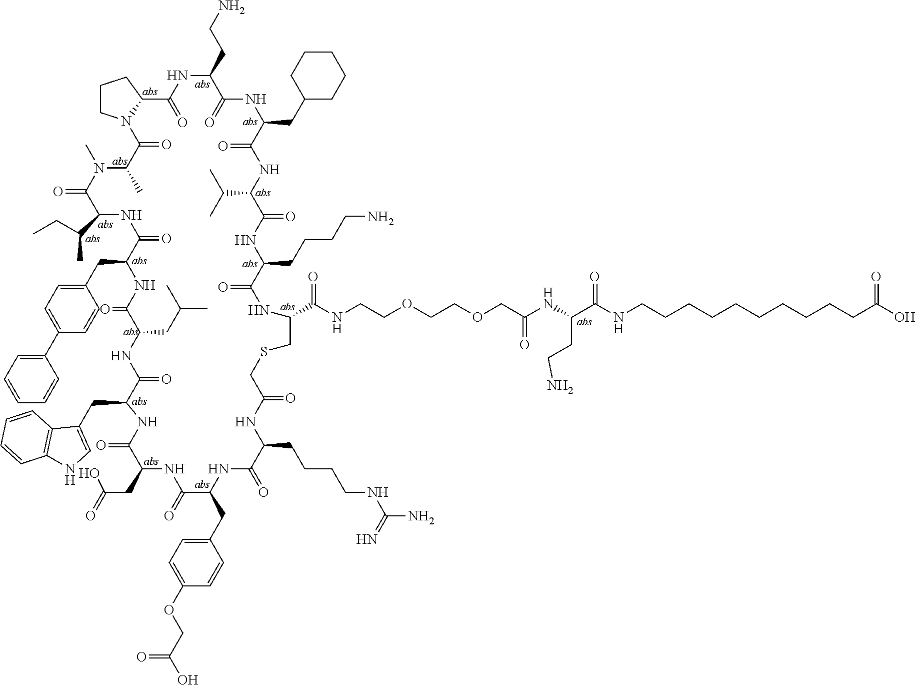 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@H]2CCCN2C(=O)[C@H](C)N(C)C1=O]

Example 2186 was prepared on a 50 μmol scale. The yield of the product was 18.7 mg, and its estimated purity by LCMS analysis was 96%. Analysis condition B: Retention time=1.48 min; ESI-MS(+) m/z [M+3H]³⁺: 799.3.

### Preparation of Example 2187

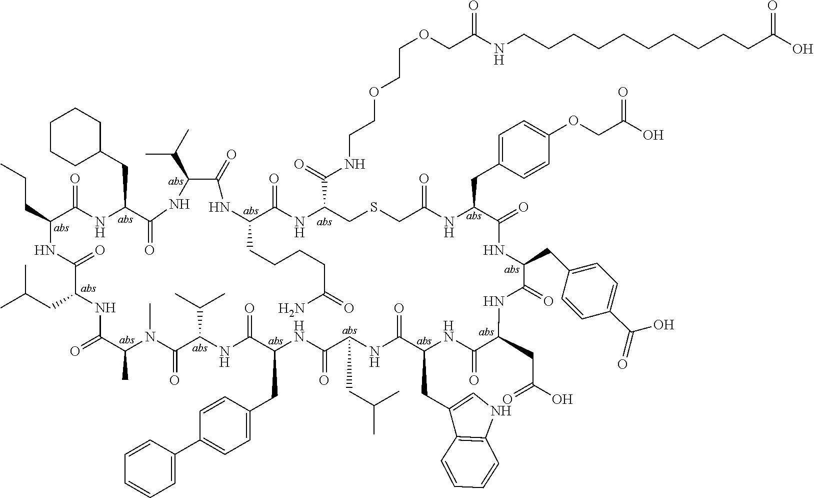 [SMILES: CCC[C@@H]1NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCC(N)=O)NC(=O)[C@H](C(C)C)NC(=O)[C@H](CC2CCCCC2)NC1=O]

Example 2187 was prepared on a 50 μmol scale. The yield of the product was 30.2 mg, and its estimated purity by LCMS analysis was 99.1%. Analysis condition A: Retention time=1.68 min; ESI-MS(+) m/z [M+2H]²⁺: 1174.1.

### Preparation of Example 2188

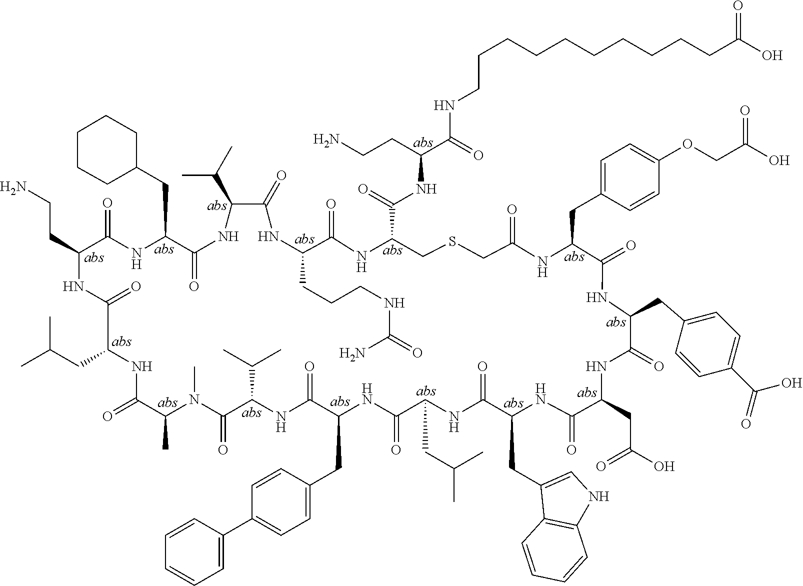 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)C[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2188 was prepared on a 50 μmol scale. The yield of the product was 21 mg, and its estimated purity by LCMS analysis was 95.1%. Analysis condition B: Retention time=1.88 min; ESI-MS(+) m/z [M+2H]²⁺: 1152.

### Preparation of Example 2189

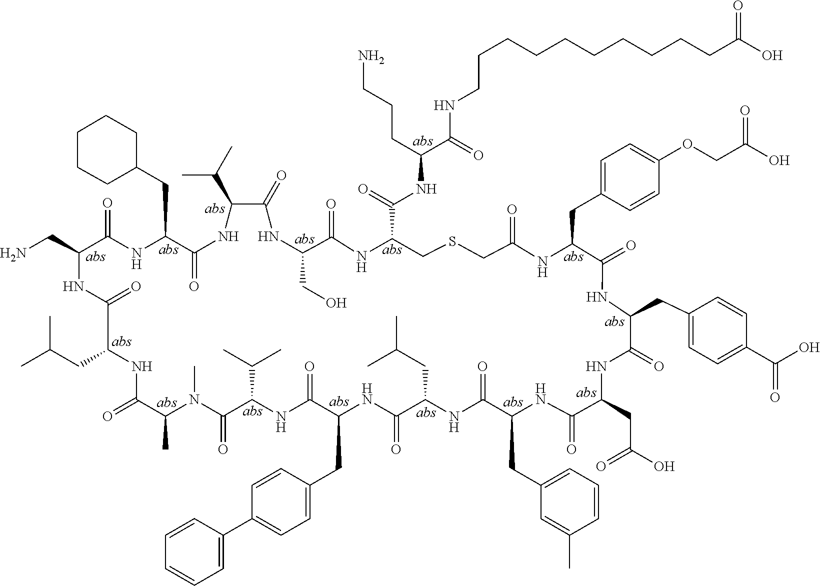 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCCN)C(=O)NCCCCCCCCCCC(=O)O)NC(=O)C[C@H](CO)NC(=O)[C@H](C(C)C)NC(=O)[C@H](CC3CCCCC3)NC(=O)[C@H](CN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2189 was prepared on a 50 μmol scale. The yield of the product was 25 mg, and its estimated purity by LCMS analysis was 97.4%. Analysis condition B: Retention time=1.75 min; ESI-MS(+) m/z [M+2H]²⁺: 1104.

### Preparation of Example 2190

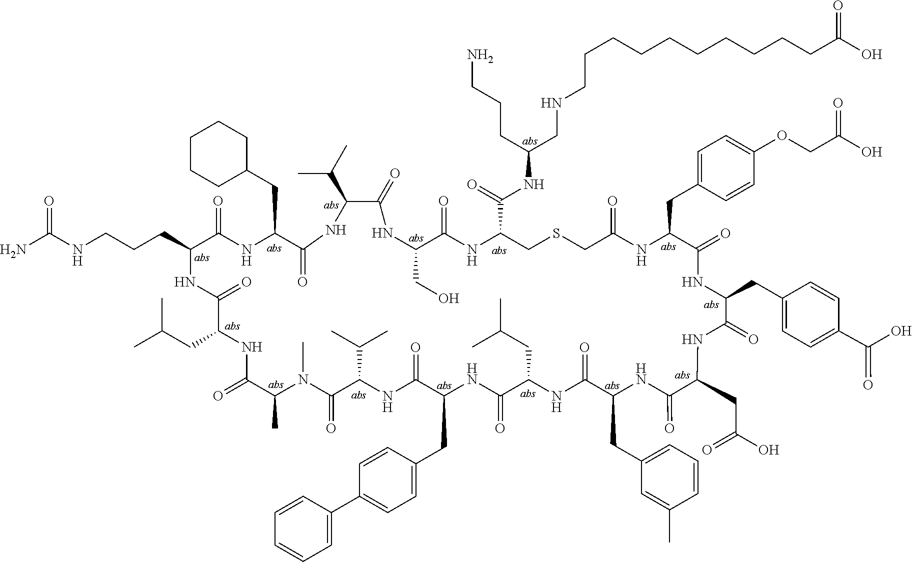 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCCN)CNCCCCCCCCCCC(=O)O)NC(=O)[C@H](CO)NC(=O)[C@H](C(C)C)NC(=O)[C@H](CC3CCCCC3)NC(=O)[C@H](CCCNC(N)=O)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2190 was prepared on a 50 μmol scale. The yield of the product was 23.7 mg, and its estimated purity by LCMS analysis was 100%. Analysis condition A: Retention time=1.52 min; ESI-MS(+) m/z [M+2H]²⁺: 1140.1.

### Preparation of Example 2191

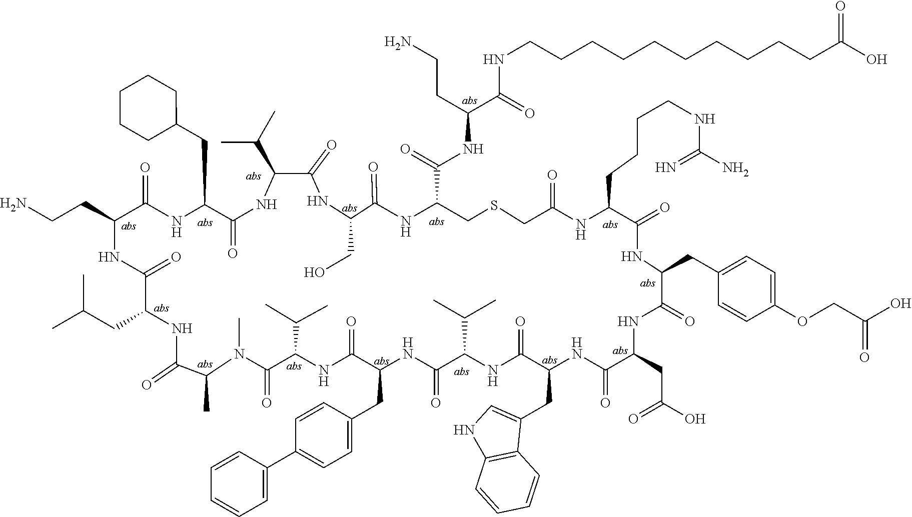 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2191 was prepared on a 50 μmol scale. The yield of the product was 14.9 mg, and its estimated purity by LCMS analysis was 99.3%. Analysis condition A: Retention time=1.4 min; ESI-MS(+) m/z [M+3H]³⁺: 733.2.

### Preparation of Example 2192

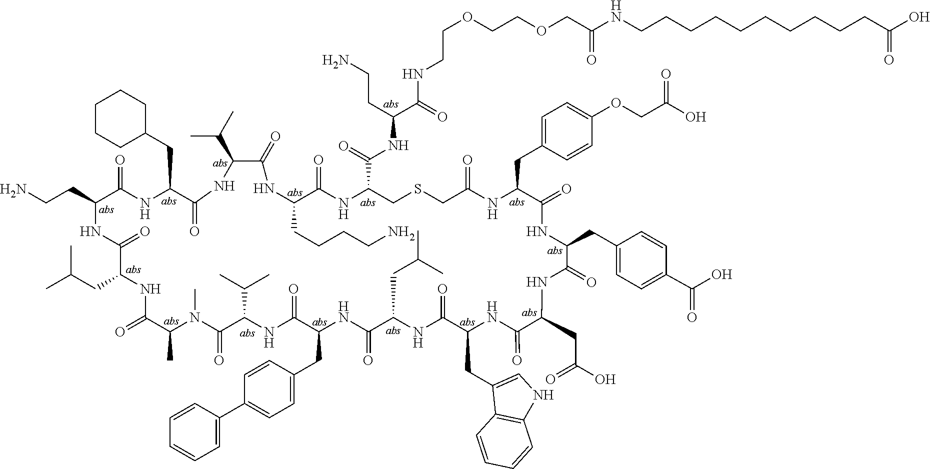 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2192 was prepared on a 50 μmol scale. The yield of the product was 15.9 mg, and its estimated purity by LCMS analysis was 95.1%. Analysis condition B: Retention time=1.84 min; ESI-MS(+) m/z [M+3H]³⁺: 806.5.

### Preparation of Example 2193

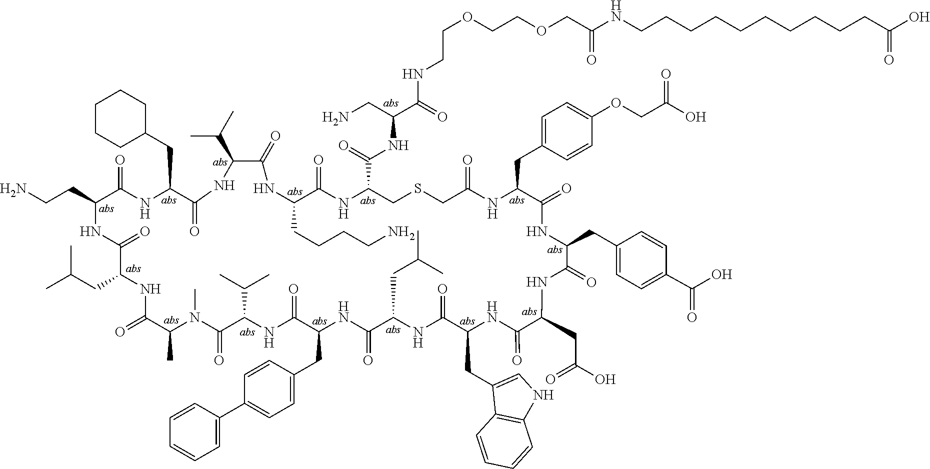 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2193 was prepared on a 50 μmol scale. The yield of the product was 21.2 mg, and its estimated purity by LCMS analysis was 95.2%. Analysis condition B: Retention time=1.83 min; ESI-MS(+) m/z [M+2H]²⁺: 1200.4.

### Preparation of Example 2194

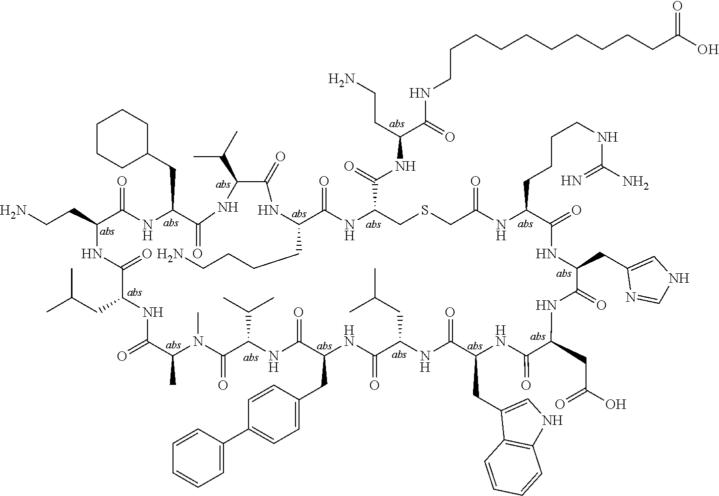 [SMILES: CC(C)C[C@@H]1NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2194 was prepared on a 50 μmol scale. The yield of the product was 10.8 mg, and its estimated purity by LCMS analysis was 100%. Analysis condition B: Retention time=1.64 min; ESI-MS(+) m/z [M+3H]³⁺: 723.2.

### Preparation of Example 2195

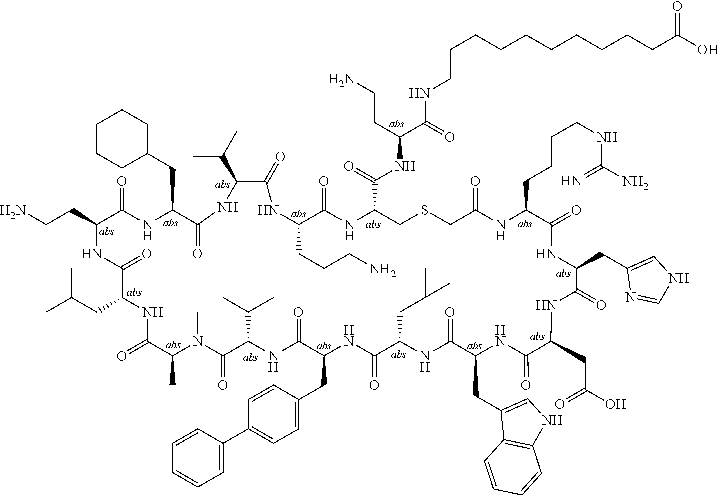 [SMILES: CC(C)C[C@@H]1NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2195 was prepared on a 50 μmol scale. The yield of the product was 17.8 mg, and its estimated purity by LCMS analysis was 95.9%. Analysis condition B: Retention time=1.51 min; ESI-MS(+) m/z [M+3H]³⁺: 718.9.

### Preparation of Example 2196

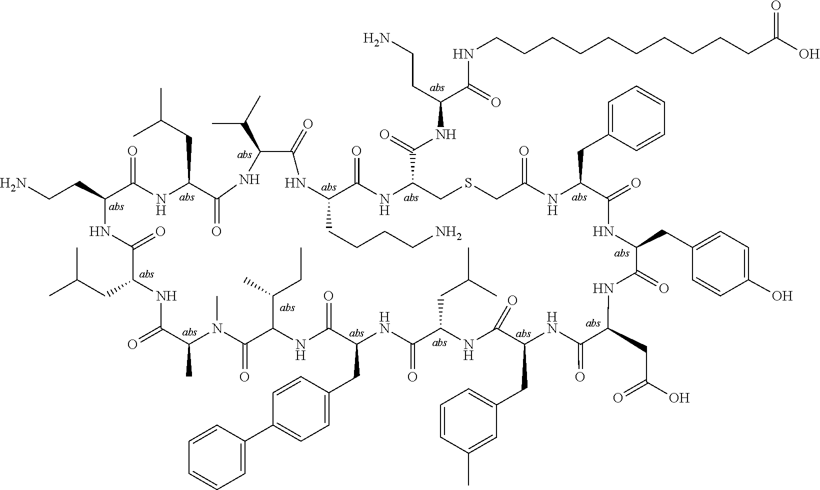 [SMILES: CC[C@@H](C)C1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC(C)C)NC(=O)[C@H](CCN)NC(=O)[C@@H](CC(C)C)NC(=O)[C@H](C)N(C)C1=O]

Example 2196 was prepared on a 50 μmol scale. The yield of the product was 2.7 mg, and its estimated purity by LCMS analysis was 100%. Analysis condition A: Retention time=1.73 min; ESI-MS(+) m/z [M+2H]²⁺: 1046.3.

### Preparation of Example 2197

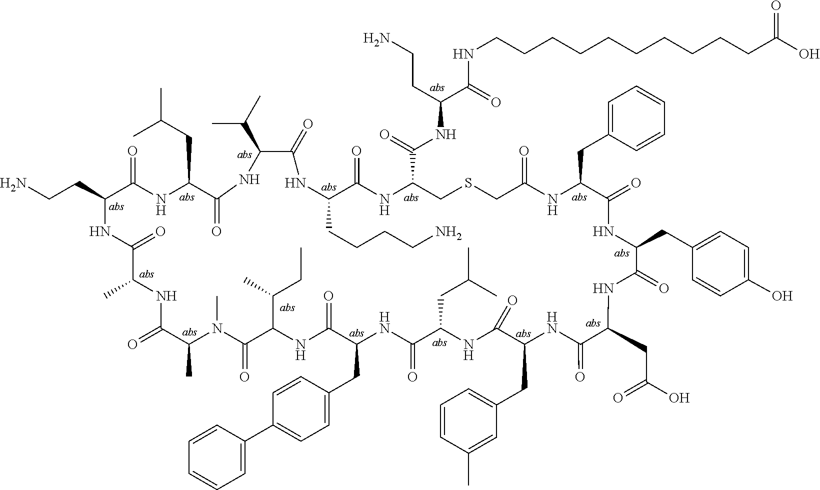 [SMILES: CC[C@@H](C)C1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC(C)C)NC(=O)[C@H](CCN)NC(=O)[C@@H](C)NC(=O)[C@H](C)N(C)C1=O]

Example 2197 was prepared on a 50 μmol scale. The yield of the product was 5.9 mg, and its estimated purity by LCMS analysis was 95.2%. Analysis condition B: Retention time=1.58 min; ESI-MS(+) m/z [M+3H]³⁺: 693.1.

### Preparation of Example 2198

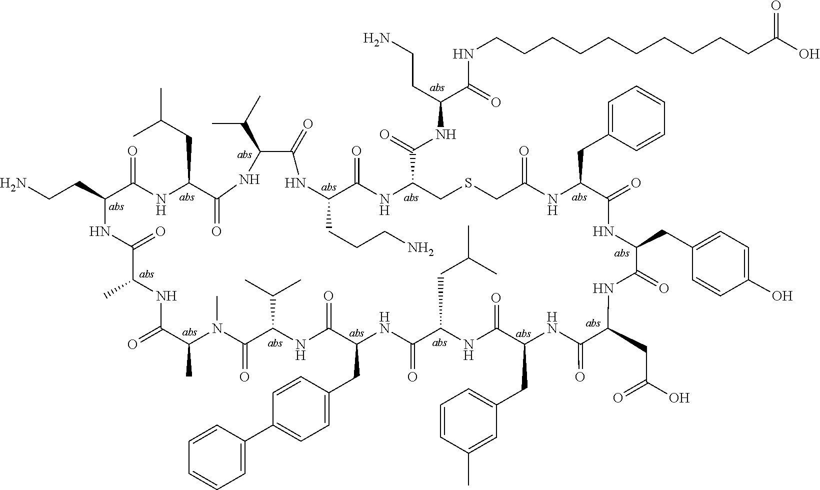 [SMILES: Cc1cccc(C[C@@H]2NC(=O)[C@H](CC(=O)O)NC(=O)[C@H](Cc3ccc(O)cc3)NC(=O)[C@H](Cc3ccccc3)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2198 was prepared on a 50 μmol scale. The yield of the product was 9.7 mg, and its estimated purity by LCMS analysis was 97.6%. Analysis condition A: Retention time=1.74 min; ESI-MS(+) m/z [M+3H]³⁺: 684.1.

### Preparation of Example 2199

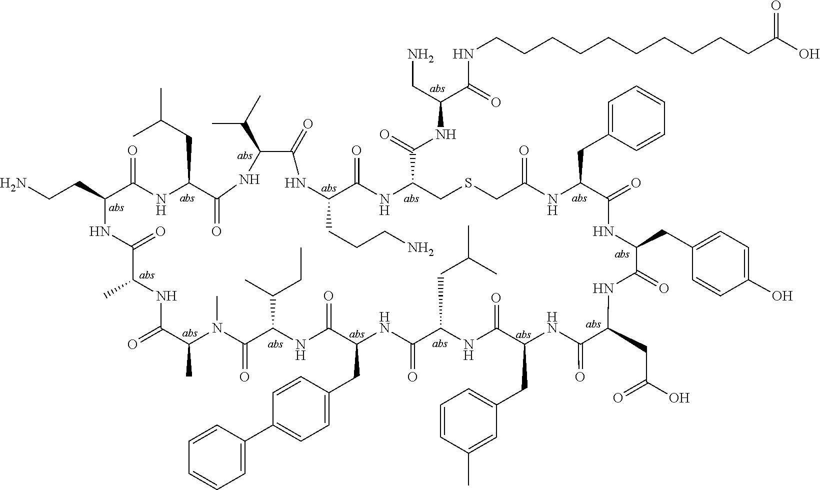 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](CN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](C)NC(=O)[C@H](C)N(C)C1=O]

Example 2199 was prepared on a 50 μmol scale. The yield of the product was 9.7 mg, and its estimated purity by LCMS analysis was 97.4%. Analysis condition B: Retention time=1.67 min; ESI-MS(+) m/z [M+3H]³⁺: 684.

### Preparation of Example 2200

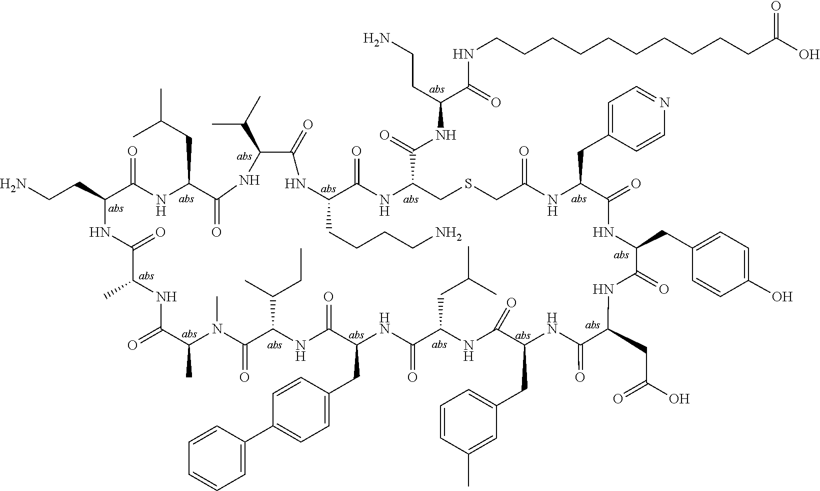 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC(C)C)NC(=O)[C@H](CCN)NC(=O)[C@@H](C)NC(=O)[C@H](C)N(C)C1=O]

Example 2200 was prepared on a 50 μmol scale. The yield of the product was 3.6 mg, and its estimated purity by LCMS analysis was 95.3%. Analysis condition A: Retention time=1.47 min; ESI-MS(+) m/z [M+2H]²⁺: 1040.1.

### Preparation of Example 2201

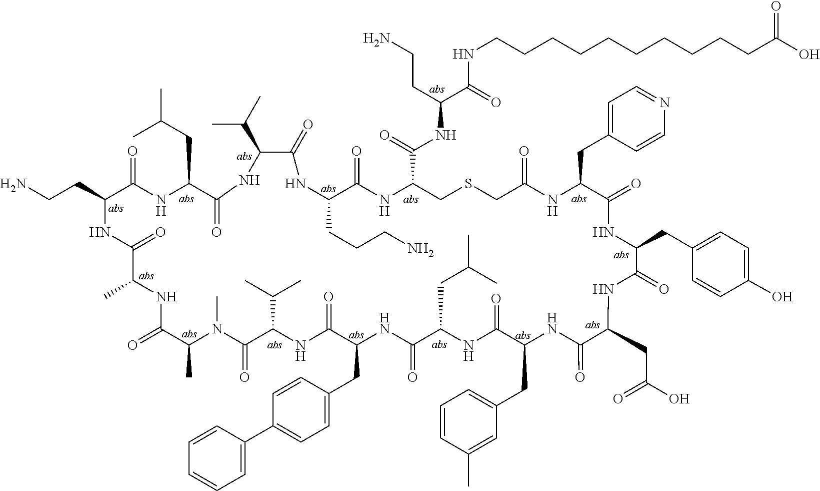 [SMILES: Cc1cccc(C[C@@H]2NC(=O)[C@H](CC(=O)O)NC(=O)[C@H](Cc3ccc(O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2201 was prepared on a 50 μmol scale. The yield of the product was 7.3 mg, and its estimated purity by LCMS analysis was 100%. Analysis condition A: Retention time=1.47 min; ESI-MS(+) m/z [M+2H]²⁺: 1026.1.

### Preparation of Example 2202

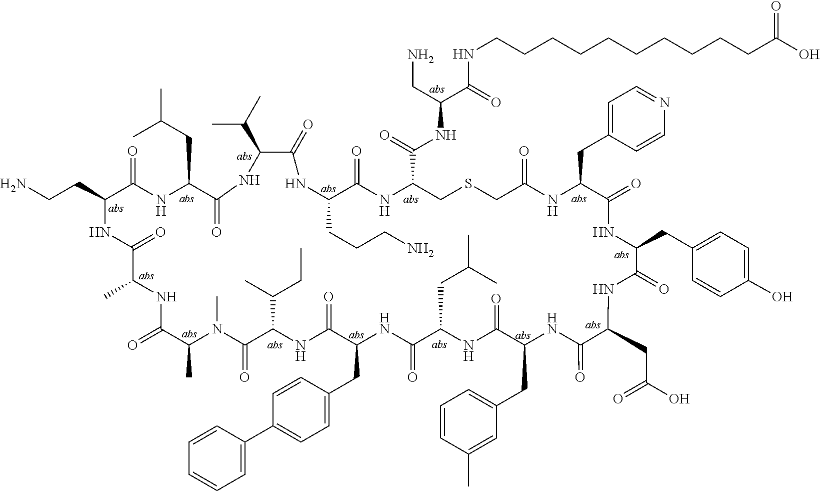 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](C)NC(=O)[C@H](C)N(C)C1=O]

Example 2202 was prepared on a 50 μmol scale. The yield of the product was 6.2 mg, and its estimated purity by LCMS analysis was 100%. Analysis condition A: Retention time=1.53 min; ESI-MS(+) m/z [M+2H]²⁺: 1025.9.

### Preparation of Example 2203

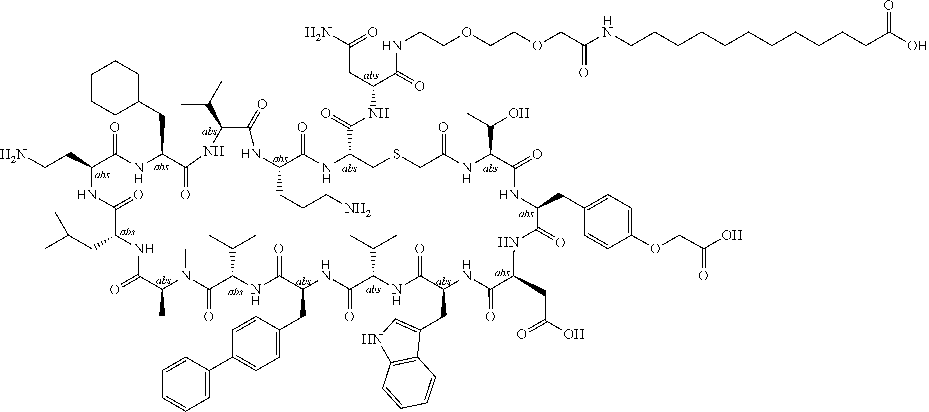 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Example 2203 was prepared on a 50 μmol scale. The yield of the product was 43.7 mg, and its estimated purity by LCMS analysis was 90.1%. Analysis condition A: Retention time=1.62 min; ESI-MS(+) m/z [M+3H]³⁺: 776.5.

### Preparation of Example 2204

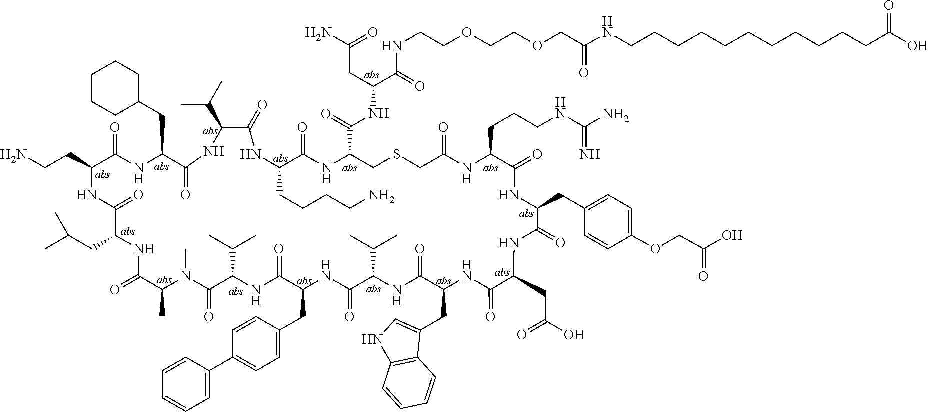 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCOCCOCC(=O)NCCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Example 2204 was prepared on a 50 μmol scale. The yield of the product was 27.9 mg, and its estimated purity by LCMS analysis was 95.3%. Analysis condition B: Retention time=1.6 min; ESI-MS(+) m/z [M+2H]²⁺: 1199.3.

### Preparation of Example 2205

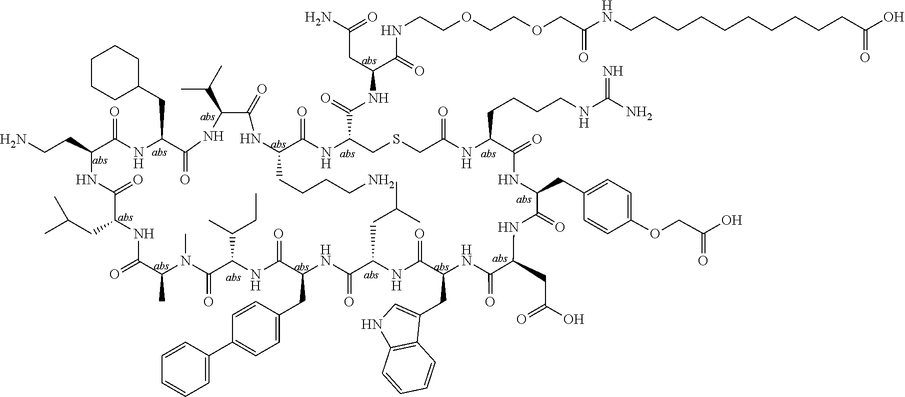 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2205 was prepared on a 50 μmol scale. The yield of the product was 11.3 mg, and its estimated purity by LCMS analysis was 99.2%. Analysis condition B: Retention time=1.53 min; ESI-MS(+) m/z [M+2H]²⁺: 1185.2.

### Preparation of Example 2206

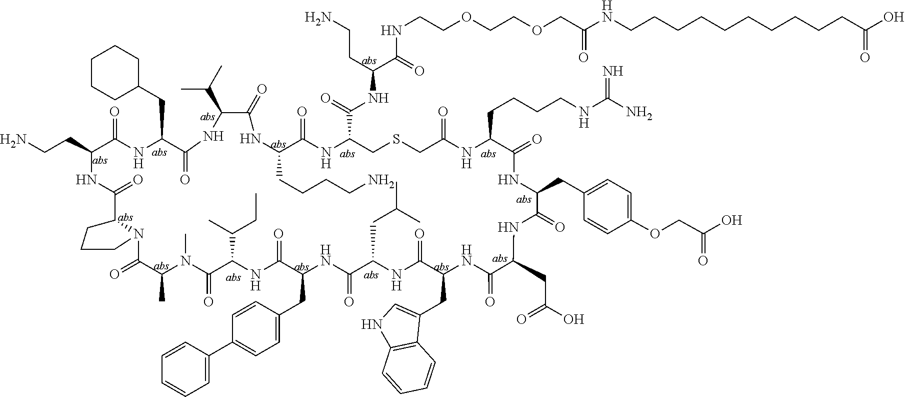 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@H]2CCCN2C(=O)[C@H](C)N(C)C1=O]

Example 2206 was prepared on a 50 μmol scale. The yield of the product was 16.5 mg, and its estimated purity by LCMS analysis was 99.5%. Analysis condition A: Retention time=1.42 min; ESI-MS(+) m/z [M+3H]³⁺: 799.

### Preparation of Example 2207

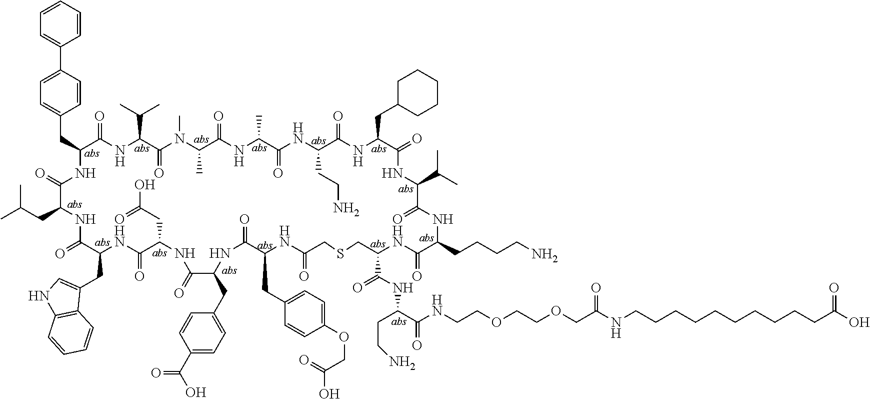 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](C)NC(=O)[C@H](C)N(C)C(=O)[C@H](C(C)C)NC(=O)[C@H](Cc2ccc(-c3ccccc3)cc2)NC1=O]

Example 2207 was prepared on a 50 μmol scale. The yield of the product was 22.9 mg, and its estimated purity by LCMS analysis was 97.9%. Analysis condition B: Retention time=1.63 min; ESI-MS(+) m/z [M+3H]³⁺: 792.6.

### Preparation of Example 2208

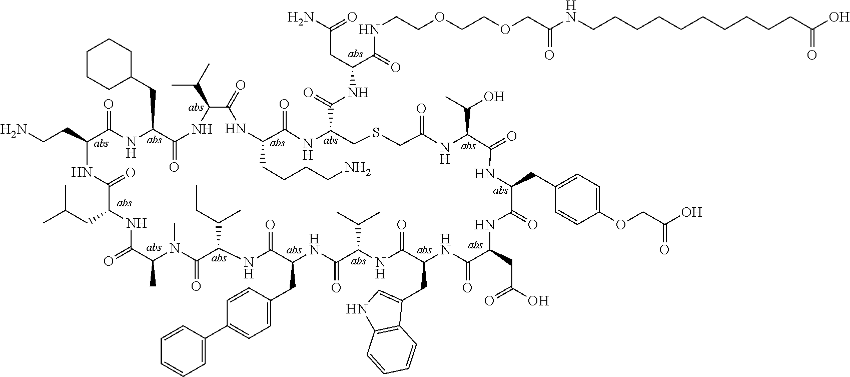 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)NCCCCCCCCCCC(=O)O)NC(=O)C[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2208 was prepared on a 50 μmol scale. The yield of the product was 26.1 mg, and its estimated purity by LCMS analysis was 95.3%. Analysis condition B: Retention time=2.52 min; ESI-MS(+) m/z [M+2H]²⁺: 1172.

### Preparation of Example 2209

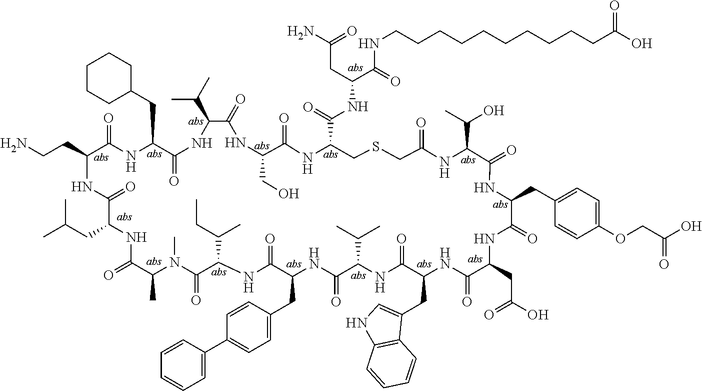 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)C[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2209 was prepared on a 50 μmol scale. The yield of the product was 15.2 mg, and its estimated purity by LCMS analysis was 98.5%. Analysis condition A: Retention time=1.62 min; ESI-MS(+) m/z [M+2H]²⁺: 1078.3.

### Preparation of Example 2210

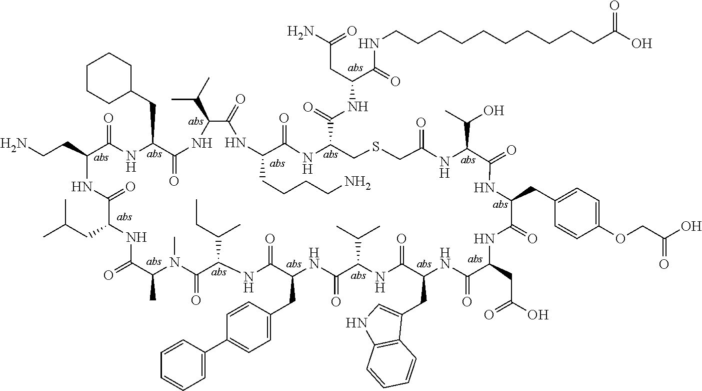 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)C[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2210 was prepared on a 50 μmol scale. The yield of the product was 20.6 mg, and its estimated purity by LCMS analysis was 95.6%. Analysis condition B: Retention time=1.69 min; ESI-MS(+) m/z [M+2H]²⁺: 1099.3.

### Preparation of Example 2211

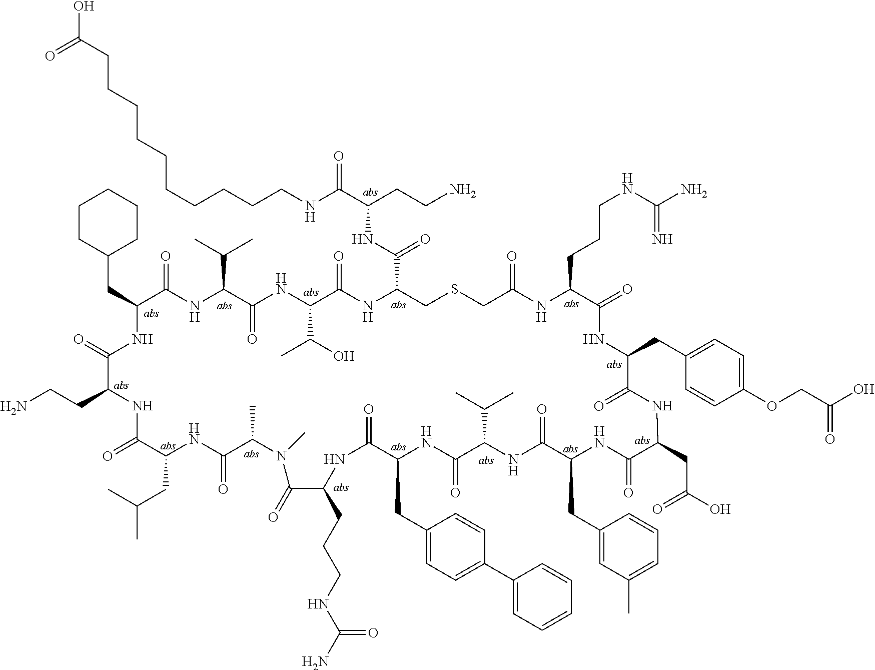 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2211 was prepared on a 50 μmol scale. The yield of the product was 22.9 mg, and its estimated purity by LCMS analysis was 95%. Analysis condition A: Retention time=1.62 min; ESI-MS(+) m/z [M+3H]³⁺: 744.2.

### Preparation of Example 2212

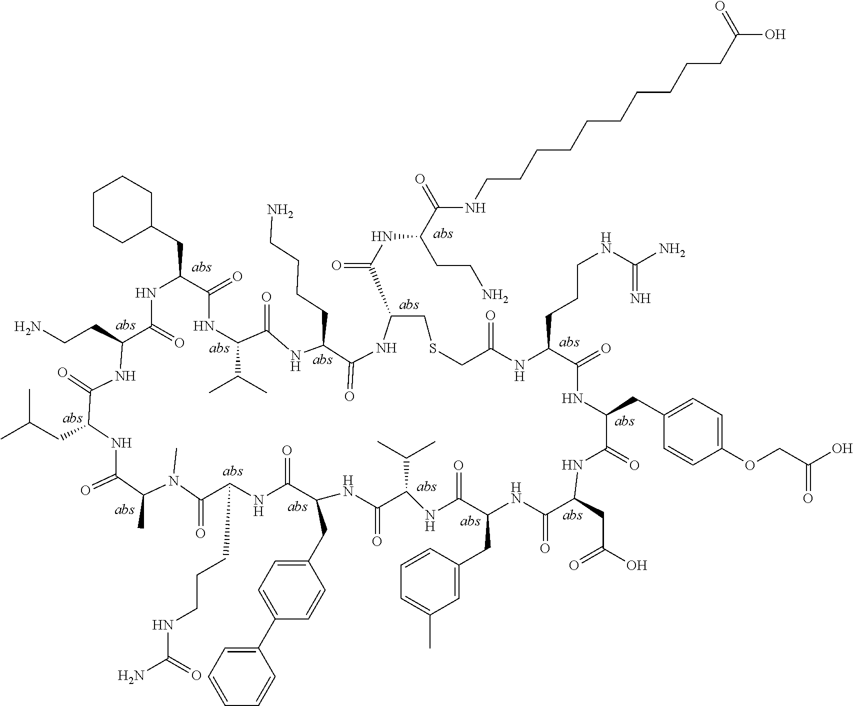 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2212 was prepared on a 50 μmol scale. The yield of the product was 16.1 mg, and its estimated purity by LCMS analysis was 94%. Analysis condition B: Retention time=1.62 min; ESI-MS(+) m/z [M+2H]²⁺: 1129.1.

### Preparation of Example 2213

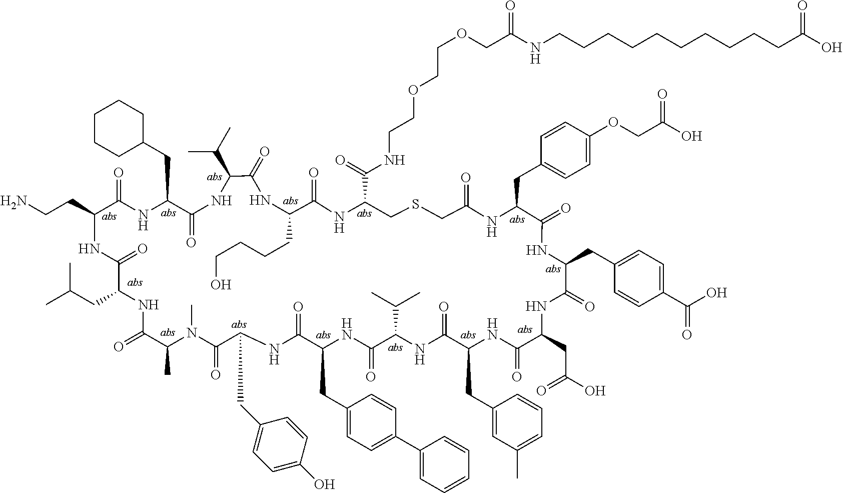 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)C[C@H](CCCCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 2213 was prepared on a 50 μmol scale. The yield of the product was 21.1 mg, and its estimated purity by LCMS analysis was 96.6%. Analysis condition A: Retention time=1.62 min; ESI-MS(+) m/z [M+2H]²⁺: 1173.

### Preparation of Example 2214

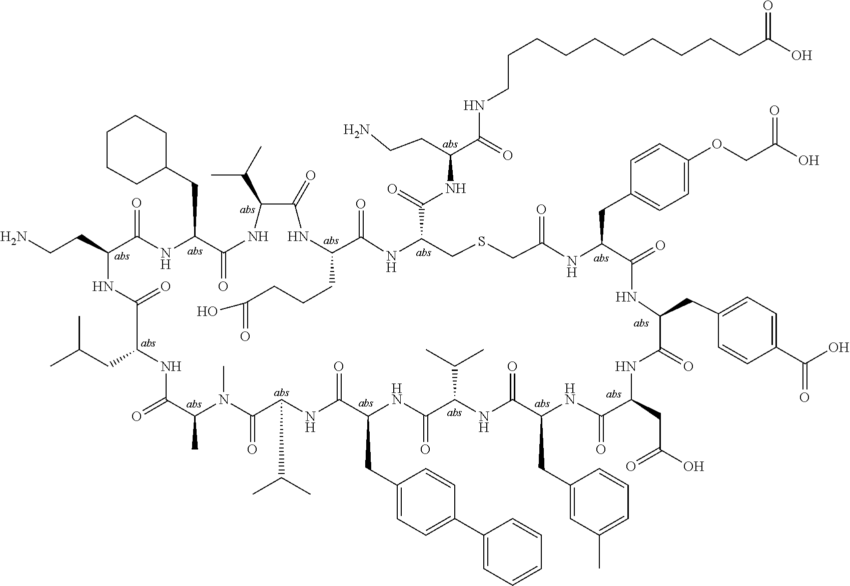 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)C[C@H](CCCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2214 was prepared on a 50 μmol scale. The yield of the product was 12.7 mg, and its estimated purity by LCMS analysis was 97.4%. Analysis condition A: Retention time=1.5 min; ESI-MS(+) m/z [M+2H]²⁺: 1125.1.

### Preparation of Example 2215

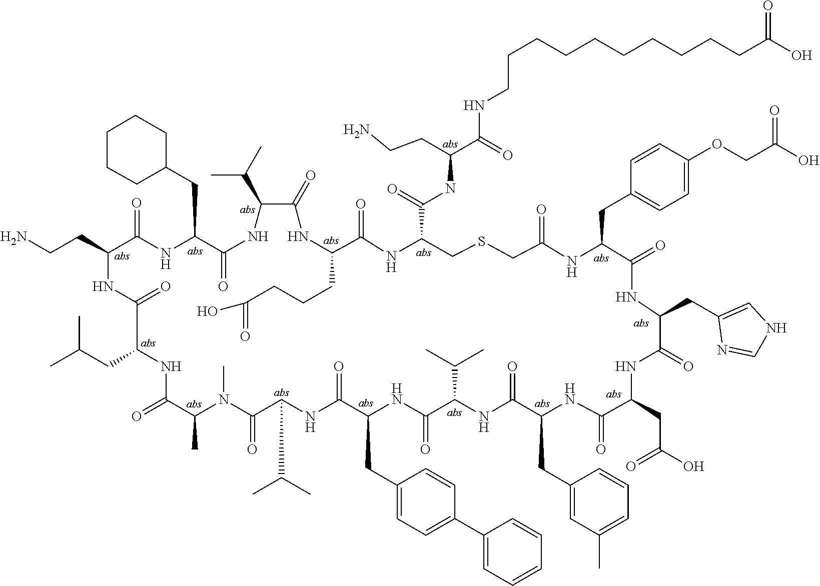 [SMILES: Cc1cccc(C[C@@H]2NC(=O)[C@H](CC(=O)O)NC(=O)[C@H](Cc3c[nH]cn3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)C[C@H](CCCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2215 was prepared on a 50 μmol scale. The yield of the product was 22.2 mg, and its estimated purity by LCMS analysis was 100%. Analysis condition A: Retention time=1.56 min; ESI-MS(+) m/z [M+2H]²⁺: 1098.1.

### Preparation of Example 2216

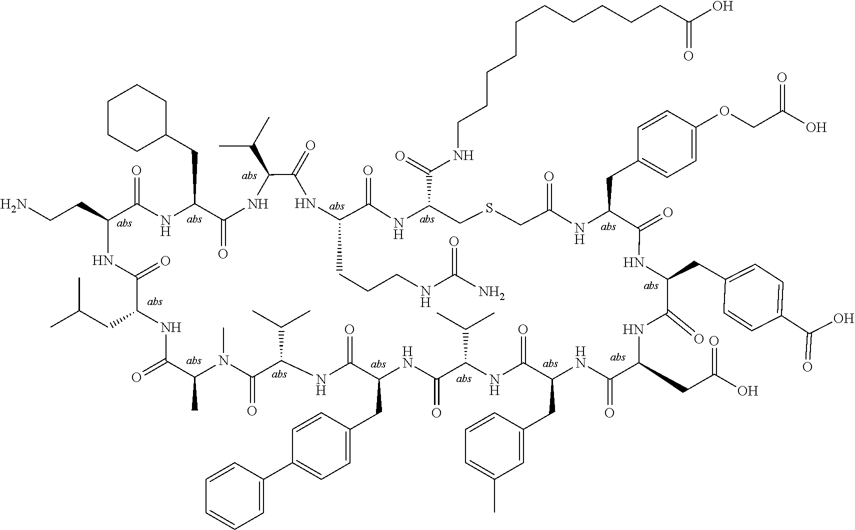 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)C[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2216 was prepared on a 50 μmol scale. The yield of the product was 49.9 mg, and its estimated purity by LCMS analysis was 95.6%. Analysis condition A: Retention time=1.68 min; ESI-MS(+) m/z [M+2H]²⁺: 1081.9.

### Preparation of Example 2217

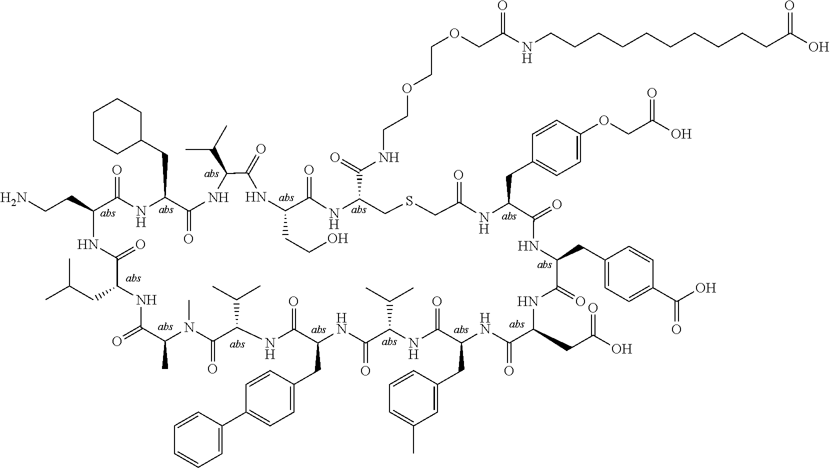 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)C[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2217 was prepared on a 50 μmol scale. The yield of the product was 25 mg, and its estimated purity by LCMS analysis was 100%. Analysis condition A: Retention time=1.64 min; ESI-MS(+) m/z [M+2H]²⁺: 1126.4.

### Preparation of Example 2218

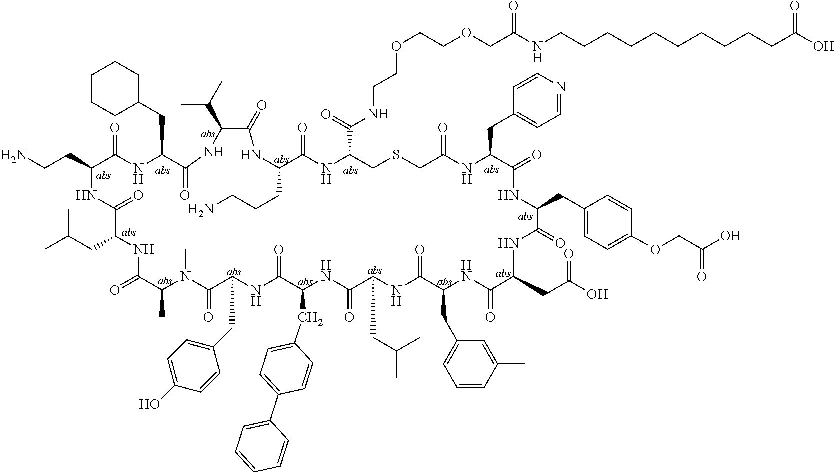 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CC(C)C)NC2=O)c1]

Example 2218 was prepared on a 50 μmol scale. The yield of the product was 20.2 mg, and its estimated purity by LCMS analysis was 96.4%. Analysis condition B: Retention time=1.79 min; ESI-MS(+) m/z [M+3H]³⁺: 767.1.

### Preparation of Example 2219

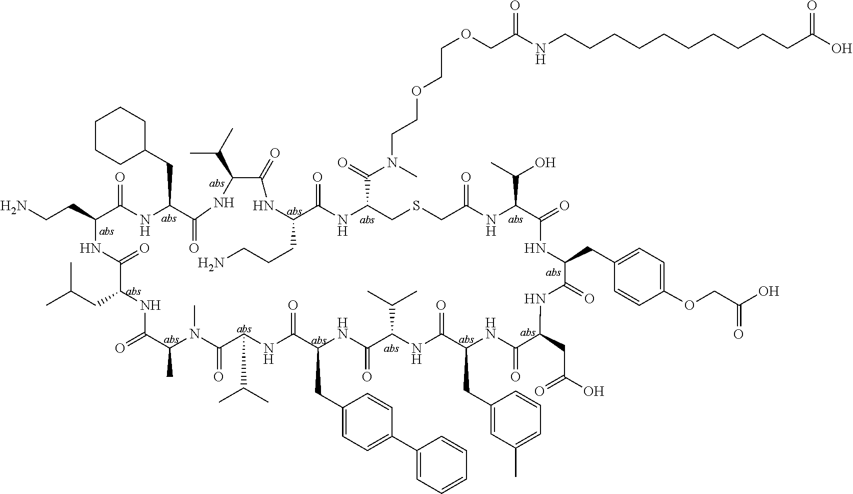 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2219 was prepared on a 50 μmol scale. The yield of the product was 5.4 mg, and its estimated purity by LCMS analysis was 96%. Analysis condition B: Retention time=1.87 min; ESI-MS(+) m/z [M+2H]²⁺: 1095.1.

### Preparation of Example 2220

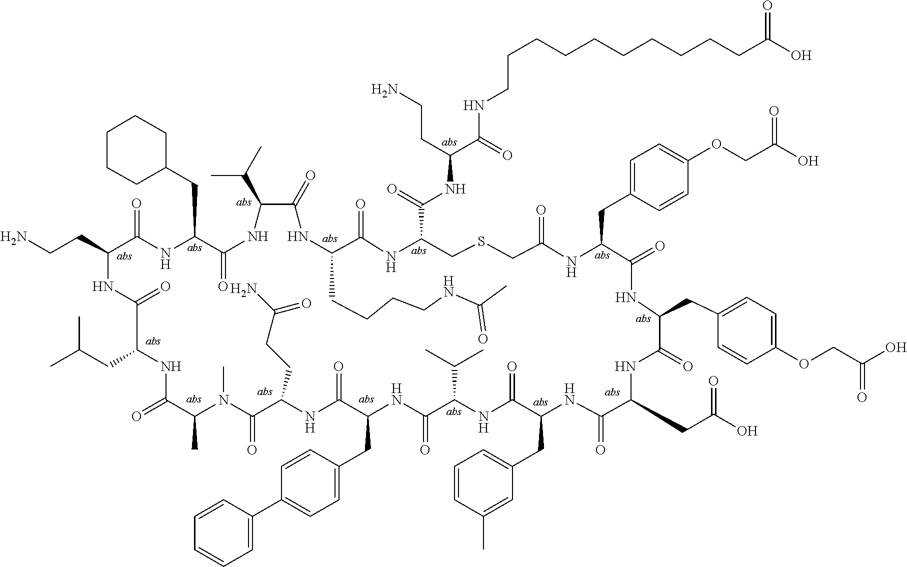 [SMILES: CC(=O)NCCCC[C@H]1CC(=O)N[C@H](C(=O)N[C@@H](CCN)C(=O)NCCCCCCCCCCC(=O)O)CSCC(=O)N[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCC(N)=O)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N1]

Example 2220 was prepared on a 80 μmol scale. The yield of the product was 30.6 mg, and its estimated purity by LCMS analysis was 95.7%. Analysis condition A: Retention time=1.49 min; ESI-MS(+) m/z [M+2H]²⁺: 1168.

### Preparation of Example 2221

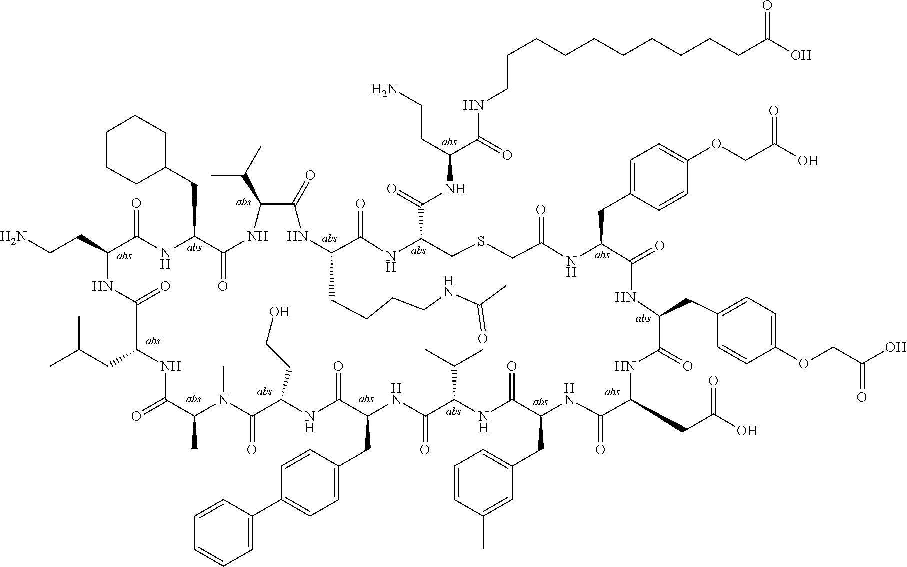 [SMILES: CC(=O)NCCCC[C@H]1CC(=O)N[C@H](C(=O)N[C@@H](CCN)C(=O)NCCCCCCCCCCC(=O)O)CSCC(=O)N[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCO)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N1]

Example 2221 was prepared on a 80 μmol scale. The yield of the product was 16.6 mg, and its estimated purity by LCMS analysis was 91.8%. Analysis condition B: Retention time=1.82 min; ESI-MS(+) m/z [M+2H]²⁺: 1155.2.

### Preparation of Example 2222

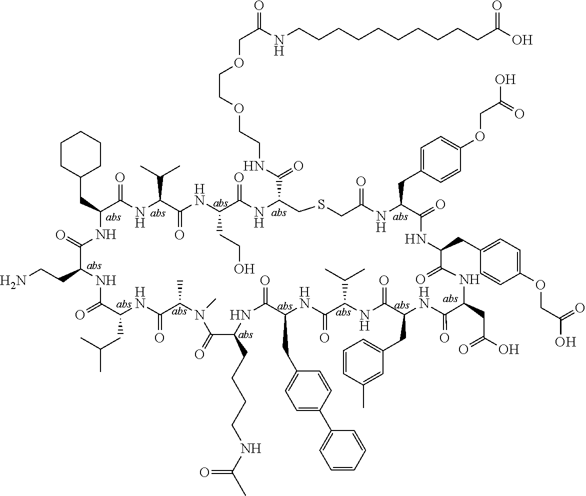 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2222 was prepared on a 80 μmol scale. The yield of the product was 49.2 mg, and its estimated purity by LCMS analysis was 95.4%. Analysis condition A: Retention time=1.6 min; ESI-MS(+) m/z [M+2H]²⁺: 1177.1.

### Preparation of Example 2223

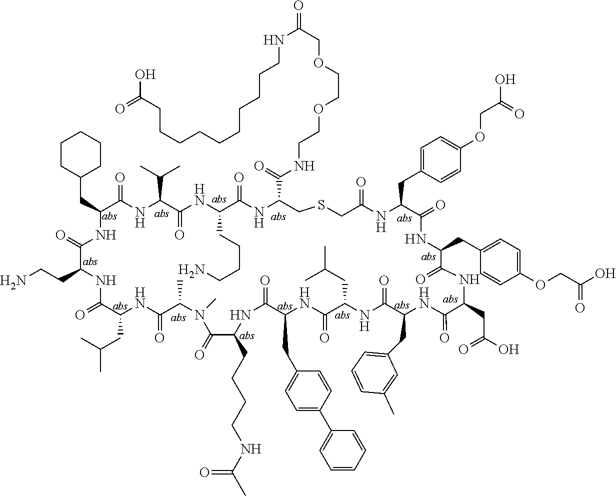 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2223 was prepared on a 80 μmol scale. The yield of the product was 3.6 mg, and its estimated purity by LCMS analysis was 95.7%. Analysis condition B: Retention time=1.87 min; ESI-MS(+) m/z [M+3H]³⁺: 798.6.

### Preparation of Example 2224

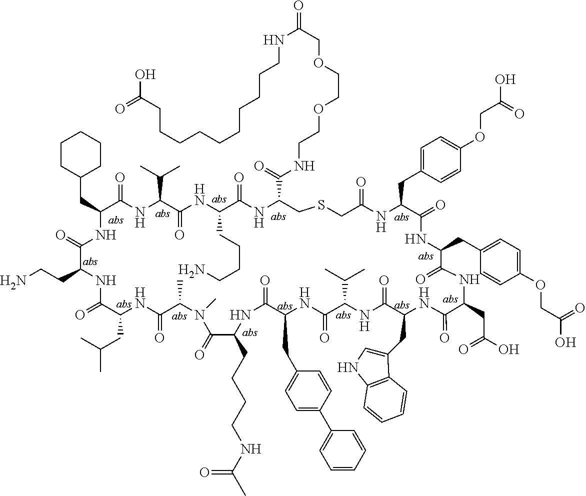 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2224 was prepared on a 80 μmol scale. The yield of the product was 24.3 mg, and its estimated purity by LCMS analysis was 95%. Analysis condition B: Retention time=1.6 min; ESI-MS(+) m/z [M+2H]²⁺: 1202.9.

### Preparation of Example 2225

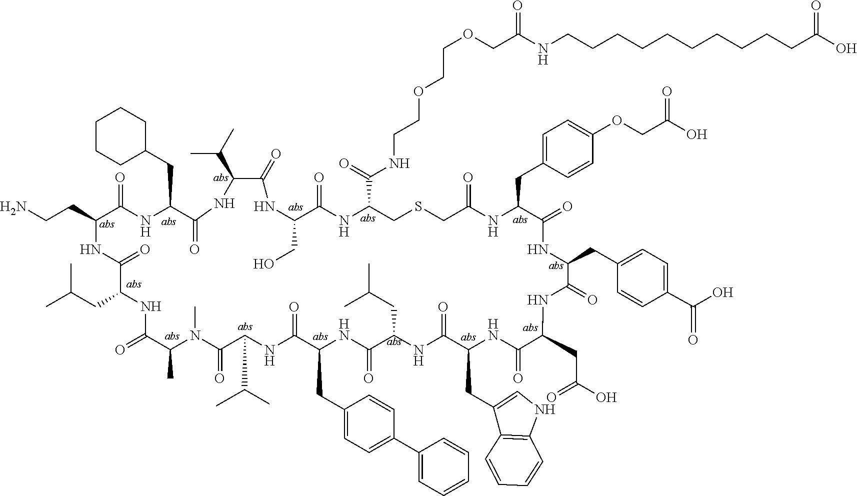 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2225 was prepared on a 50 μmol scale. The yield of the product was 36.5 mg, and its estimated purity by LCMS analysis was 100%. Analysis condition A: Retention time=1.75 min; ESI-MS(+) m/z [M+2H]²⁺: 1138.9.

### Preparation of Example 2226

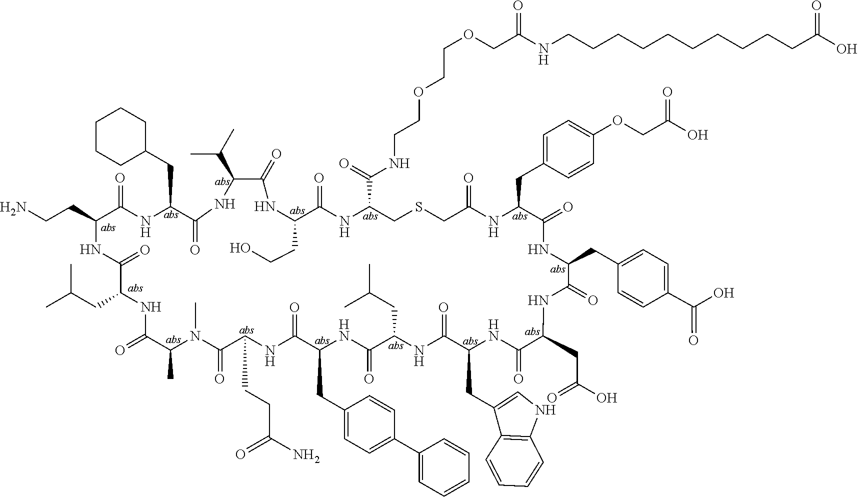 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2226 was prepared on a 50 μmol scale. The yield of the product was 51.9 mg, and its estimated purity by LCMS analysis was 94.5%. Analysis condition A: Retention time=1.7 min; ESI-MS(+) m/z [M+2H]²⁺: 1153.2.

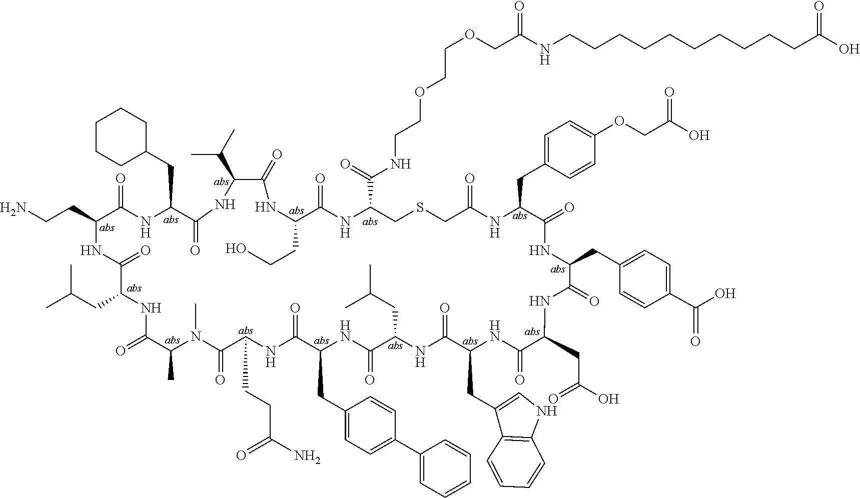 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2227 was prepared on a 50 μmol scale. The yield of the product was 6.3 mg, and its estimated purity by LCMS analysis was 97.4%. Analysis condition A: Retention time=1.51 min; ESI-MS(+) m/z [M+2H]²⁺: 1160.3.

### Preparation of Example 2228

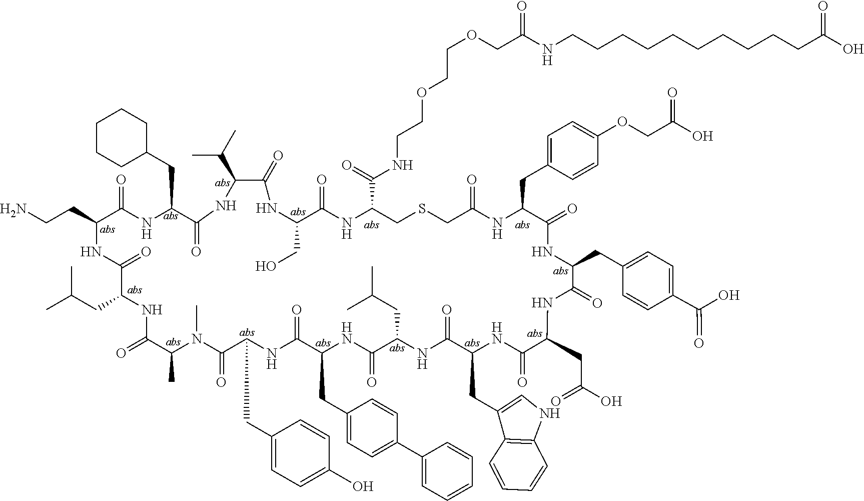 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(O)cc2)NC(=O)[C@H](Cc2ccc(-c3ccccc3)cc2)NC1=O]

Example 2228 was prepared on a 50 μmol scale. The yield of the product was 57.1 mg, and its estimated purity by LCMS analysis was 96.5%. Analysis condition A: Retention time=1.77 min; ESI-MS(+) m/z [M+2H]²⁺: 1170.8.

### Preparation of Example 2229

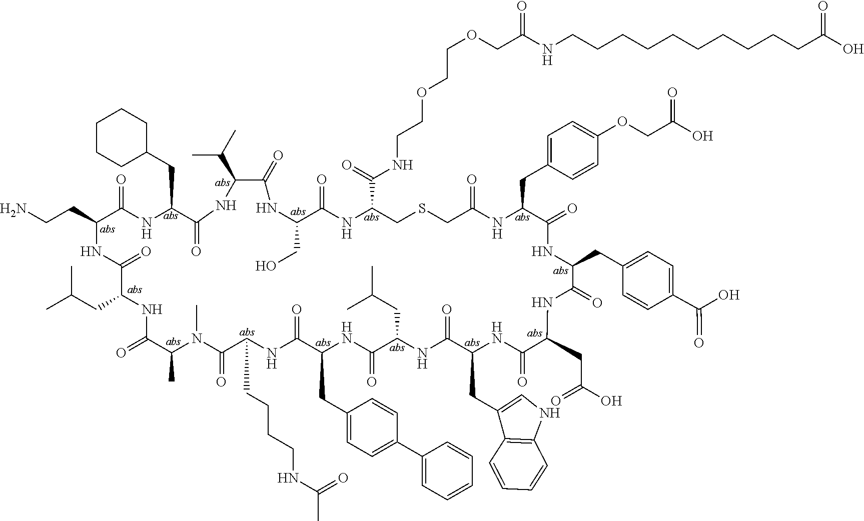 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2229 was prepared on a 50 μmol scale. The yield of the product was 63.4 mg, and its estimated purity by LCMS analysis was 92%. Analysis condition A: Retention time=1.7 min; ESI-MS(+) m/z [M+2H]²⁺: 1174.9.

### Preparation of Example 2230

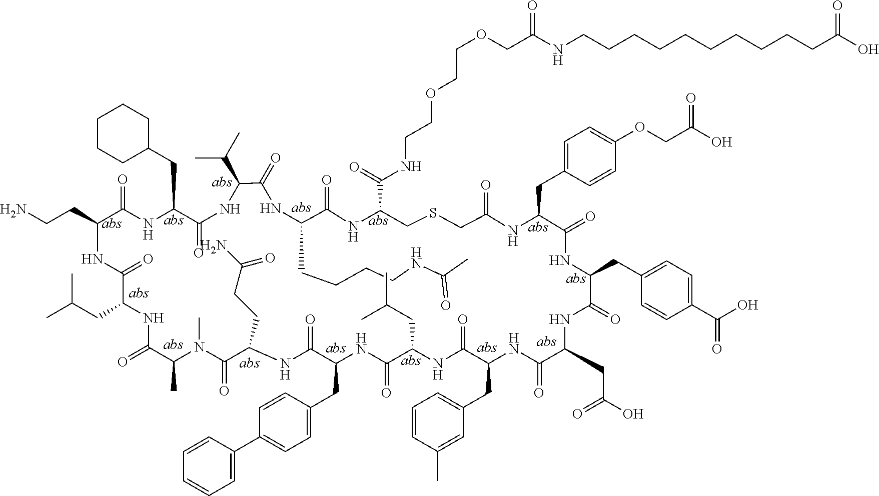 [SMILES: CC(=O)NCCCC[C@H]1CC(=O)N[C@H](C(=O)NCCOCCOCC(=O)NCCCCCCCCCCC(=O)O)CSCC(=O)N[C@@H](Cc2ccc(OCC(=O)O)cc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](CC(C)C)C(=O)N[C@@H](Cc2ccc(-c3ccccc3)cc2)C(=O)N[C@@H](CCC(N)=O)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N1]

Example 2230 was prepared on a 50 μmol scale. The yield of the product was 72 mg, and its estimated purity by LCMS analysis was 97.4%. Analysis condition B: Retention time=2.03 min; ESI-MS(+) m/z [M+2H]²⁺: 1183.

### Preparation of Example 2231

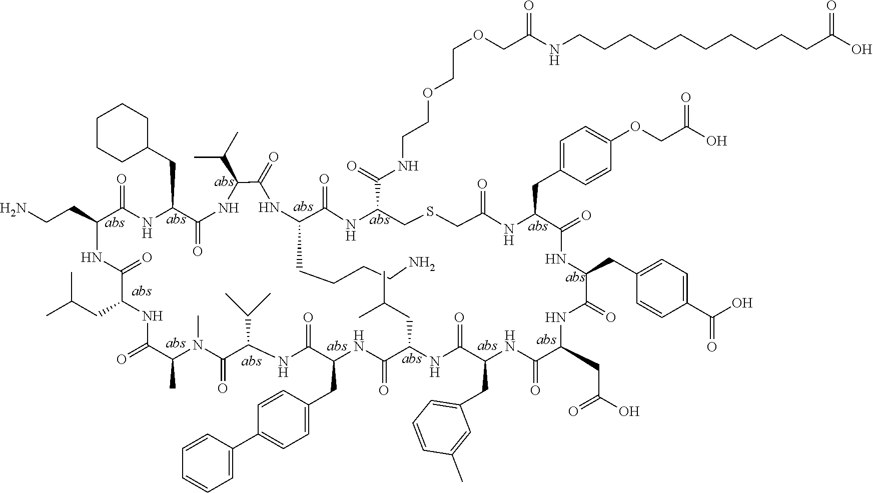 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)C[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2231 was prepared on a 50 μmol scale. The yield of the product was 65.2 mg, and its estimated purity by LCMS analysis was 97.8%. Analysis condition B: Retention time=1.9 min; ESI-MS(+) m/z [M+2H]²⁺: 1147.3.

### Preparation of Example 2232

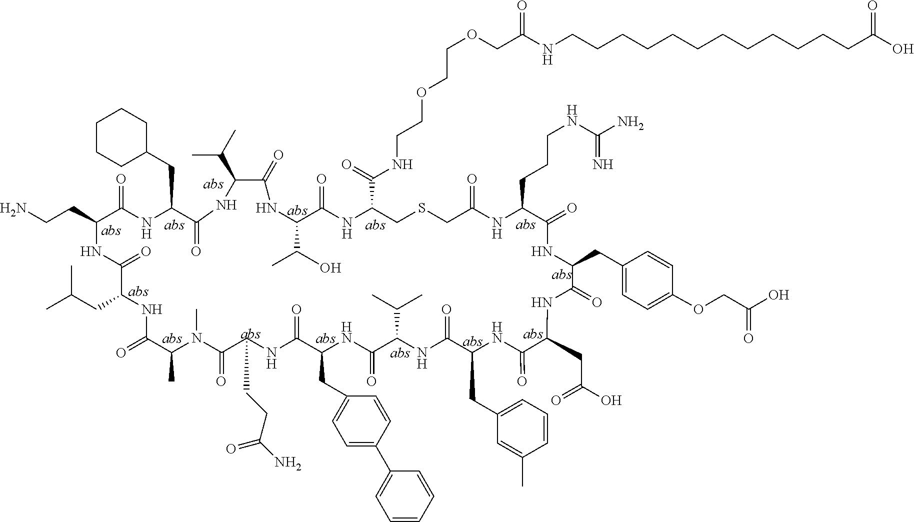 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](C(C)C)NC2=O)c1]

Example 2232 was prepared on a 50 μmol scale. The yield of the product was 41.3 mg, and its estimated purity by LCMS analysis was 96.3%. Analysis condition A: Retention time=1.79 min; ESI-MS(+) m/z [M+2H]²⁺: 1123.3.

### Preparation of Example 2233

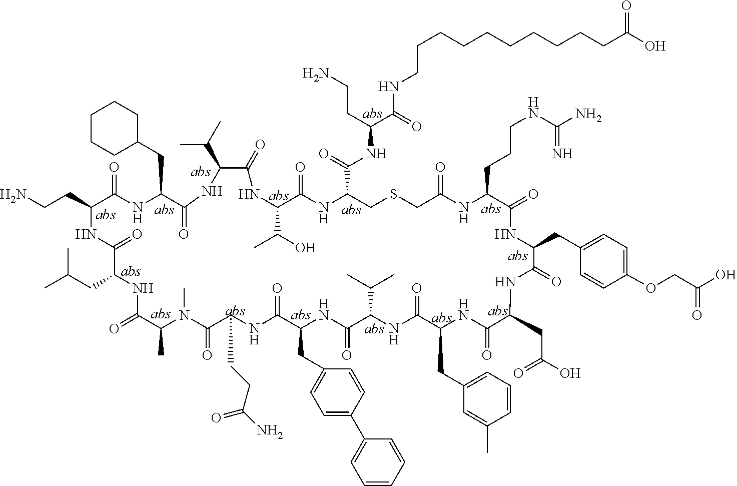 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](C(C)C)NC2=O)c1]

Example 2233 was prepared on a 50 μmol scale. The yield of the product was 39.5 mg, and its estimated purity by LCMS analysis was 94.9%. Analysis condition A: Retention time=1.67 min; ESI-MS(+) m/z [M+2H]²⁺: 1100.9.

### Preparation of Example 2234

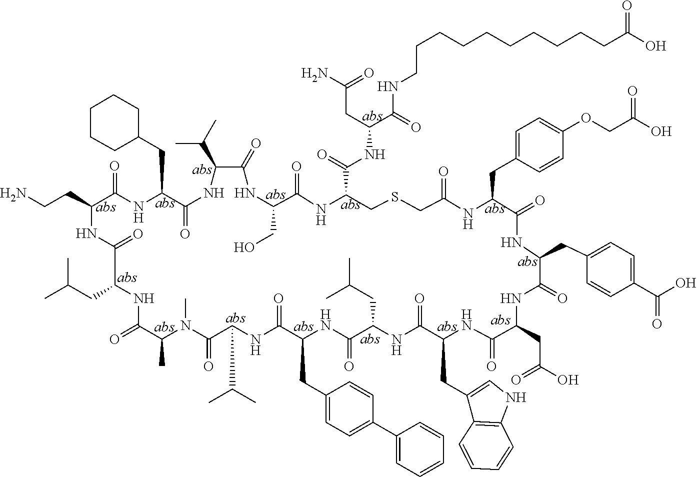 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CC(N)=O)C(=O)NCCCCCCCCCCC(=O)O)NC(=O)C[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2234 was prepared on a 50 μmol scale. The yield of the product was 81.6 mg, and its estimated purity by LCMS analysis was 95%. Analysis condition B: Retention time=2 min; ESI-MS(+) m/z [M+2H]²⁺: 1123.2.

### Preparation of Example 2235

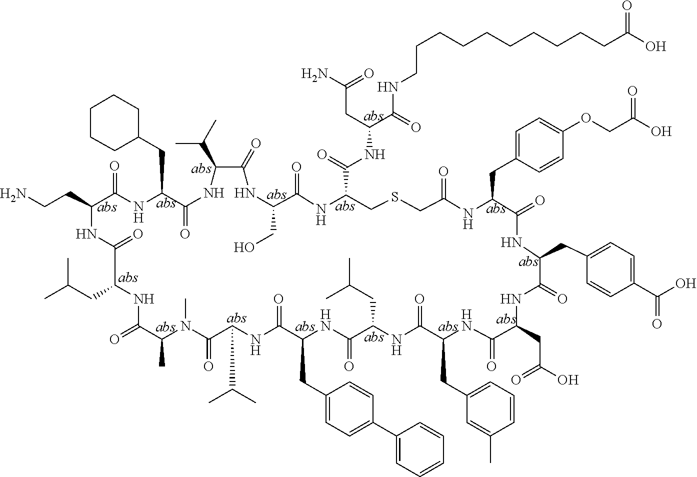 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@H](CC(N)=O)C(=O)NCCCCCCCCCCC(=O)O)NC(=O)C[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2235 was prepared on a 50 μmol scale. The yield of the product was 8.4 mg, and its estimated purity by LCMS analysis was 91.5%. Analysis condition B: Retention time=2.04 min; ESI-MS(+) m/z [M+2H]²⁺: 1111.

### Preparation of Example 2236

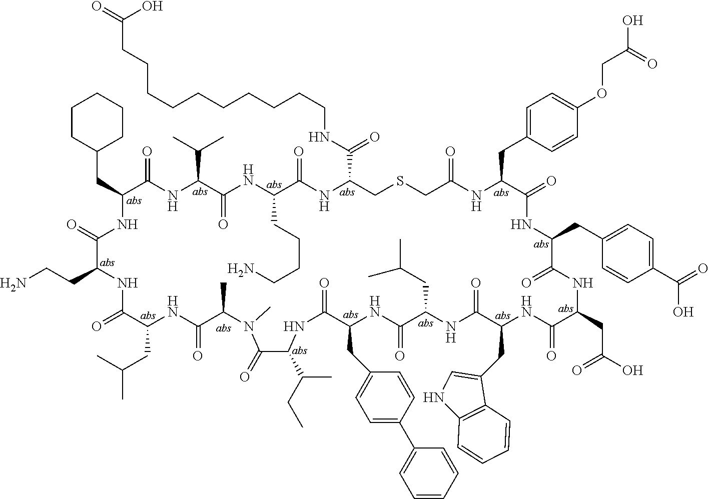 [SMILES: CCC(C)[C@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@@H](C)N(C)C1=O]

Example 2236 was prepared on a 50 μmol scale. The yield of the product was 73.2 mg, and its estimated purity by LCMS analysis was 98.6%. Analysis condition B: Retention time=1.83 min; ESI-MS(+) m/z [M+2H]²⁺: 1094.1.

### Preparation of Example 2237

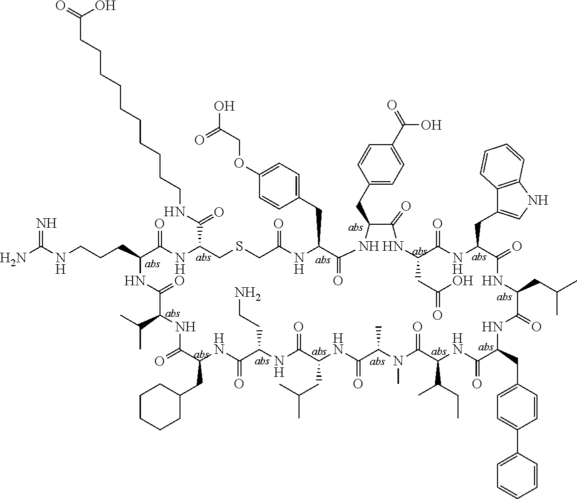 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2237 was prepared on a 50 μmol scale. The yield of the product was 41.9 mg, and its estimated purity by LCMS analysis was 93.1%. Analysis condition B: Retention time=1.92 min; ESI-MS(+) m/z [M+2H]²⁺: 1107.9.

### Preparation of Example 2238

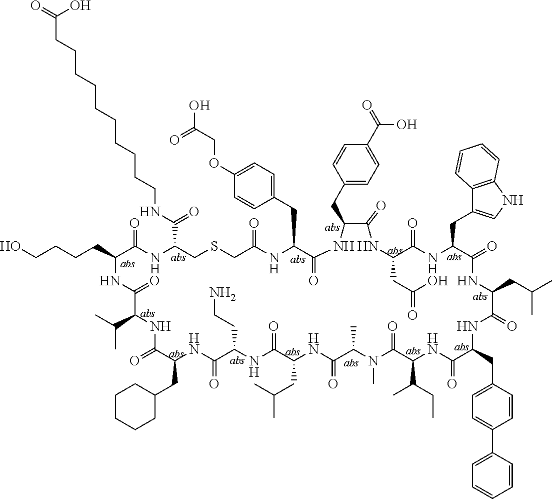 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2238 was prepared on a 50 μmol scale. The yield of the product was 40.6 mg, and its estimated purity by LCMS analysis was 95%. Analysis condition A: Retention time=1.75 min; ESI-MS(+) m/z [M+2H]²⁺: 1092.2.

### Preparation of Example 2239

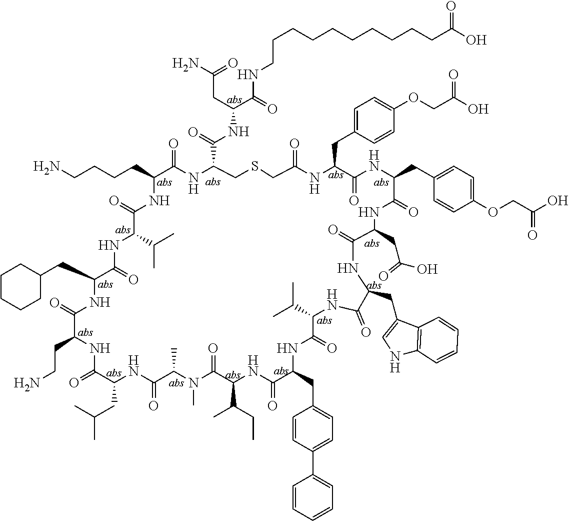 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CC(N)=O)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2239 was prepared on a 50 μmol scale. The yield of the product was 90.8 mg, and its estimated purity by LCMS analysis was 97.4%. Analysis condition A: Retention time=1.59 min; ESI-MS(+) m/z [M+2H]²⁺: 1159.1.

### Preparation of Example 2240

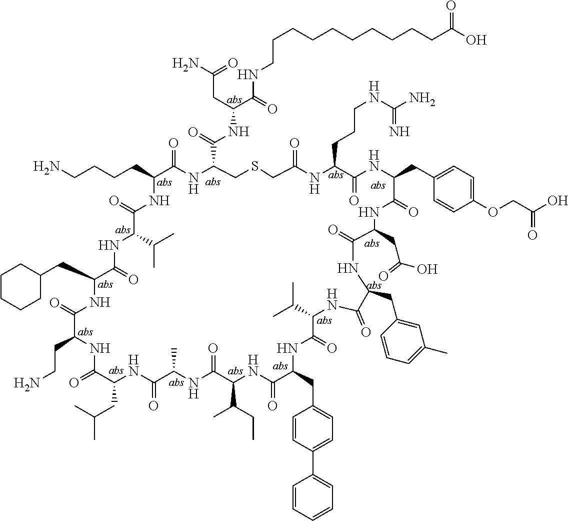 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](C)NC1=O]

Example 2240 was prepared on a 50 μmol scale. The yield of the product was 17.6 mg, and its estimated purity by LCMS analysis was 89.8%. Analysis condition A: Retention time=1.78 min; ESI-MS(+) m/z [M+2H]²⁺: 1113.9.

### Preparation of Example 2241

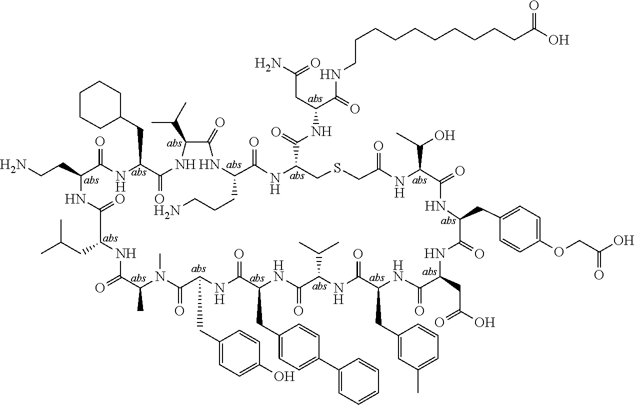 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](C(C)C)NC2=O)c1]

Example 2241 was prepared on a 50 μmol scale. The yield of the product was 53 mg, and its estimated purity by LCMS analysis was 96%. Analysis condition B: Retention time=1.83 min; ESI-MS(+) m/z [M+2H]²⁺: 1105.2.

### Preparation of Example 2242

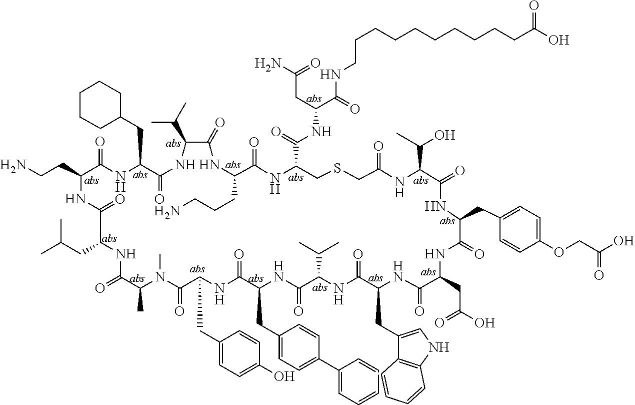 [SMILES: CC(C)C[C@H]1NC(=O)[C@H](C)N(C)C(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Example 2242 was prepared on a 50 μmol scale. The yield of the product was 30.6 mg, and its estimated purity by LCMS analysis was 100%. Analysis condition A: Retention time=1.61 min; ESI-MS(+) m/z [M+2H]²⁺: 1117.1.

### Preparation of Example 2243

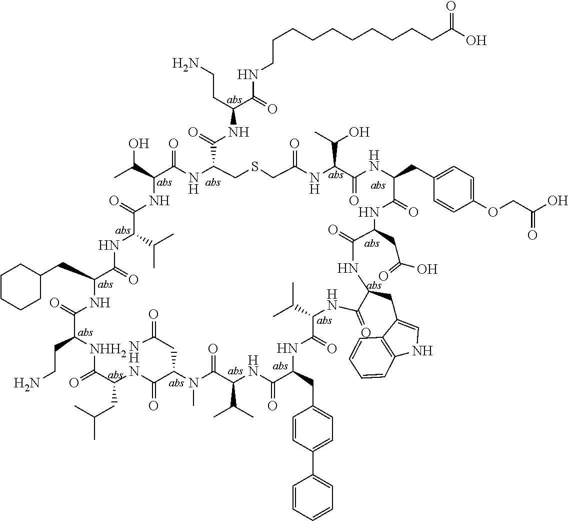

Example 2243 was prepared on a 50 μmol scale. The yield of the product was 2.5 mg, and its estimated purity by LCMS analysis was 98.4%. Analysis condition B: Retention time=1.64 min; ESI-MS(+) m/z [M+2H]²⁺: 1093.3.

### Preparation of Example 2244

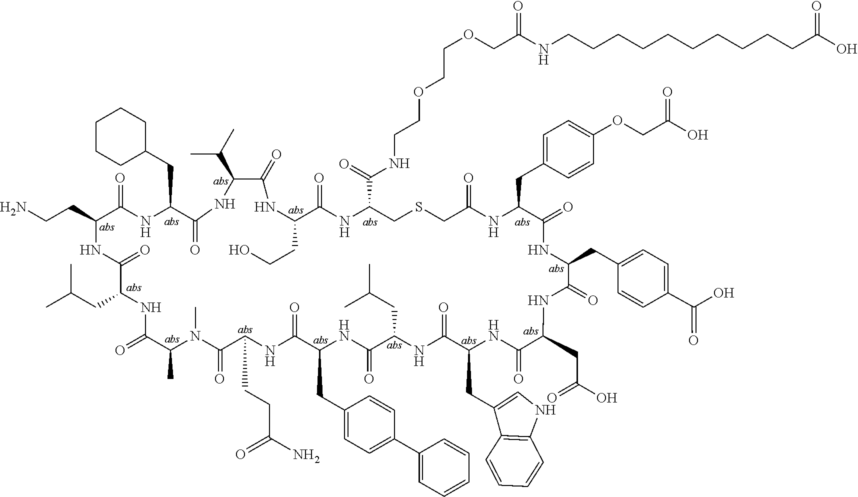 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2244 was prepared on a 50 μmol scale. The yield of the product was 13.5 mg, and its estimated purity by LCMS analysis was 99.1%. Analysis condition A: Retention time=1.51 min; ESI-MS(+) m/z [M+2H]²⁺: 1160.3.

### Preparation of Example 2245

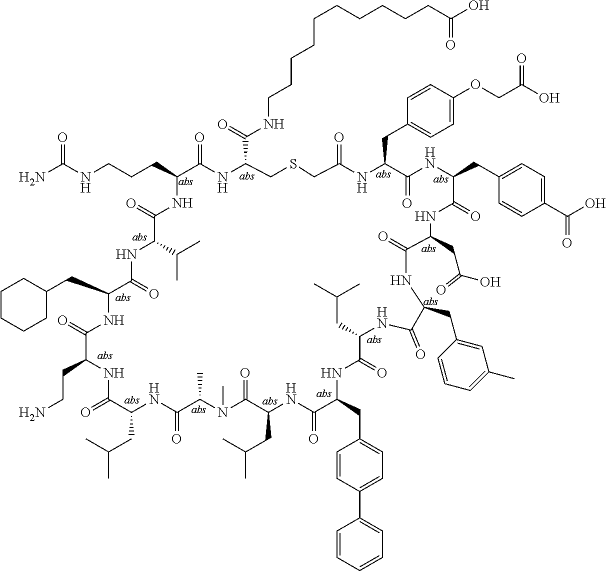 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CC(C)C)NC(=O)[C@H](Cc3ccc(-c4ccccc4)cc3)NC(=O)[C@H](CC(C)C)NC2=O)c1]

Example 2245 was prepared on a 50 μmol scale. The yield of the product was 57.2 mg, and its estimated purity by LCMS analysis was 94.7%. Analysis condition B: Retention time=2.09 min; ESI-MS(+) m/z [M+2H]²⁺: 1095.2.

### Preparation of Example 2246

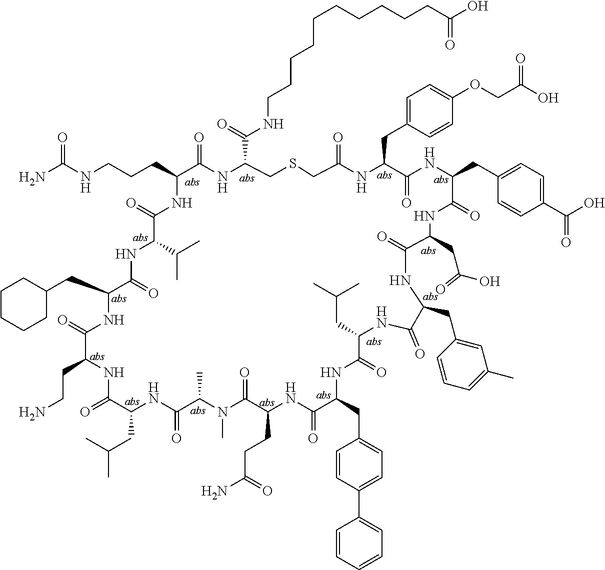 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](CC(C)C)NC2=O)c1]

Example 2246 was prepared on a 50 μmol scale. The yield of the product was 11.2 mg, and its estimated purity by LCMS analysis was 95%. Analysis condition A: Retention time=1.75 min; ESI-MS(+) m/z [M+2H]²⁺: 1103.1.

### Preparation of Example 2247

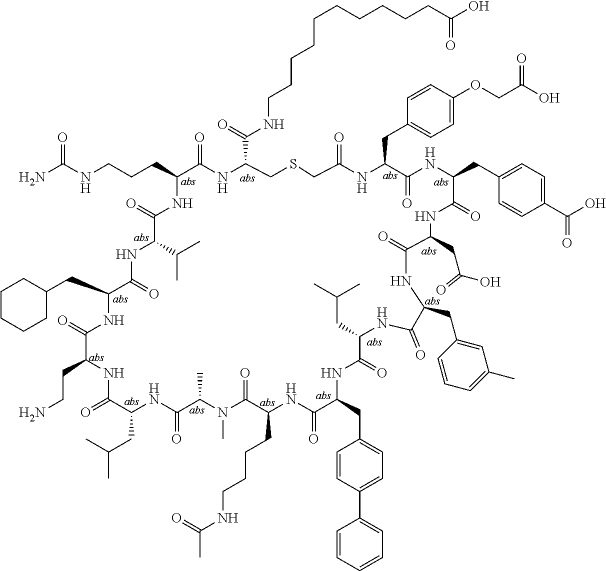 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2247 was prepared on a 50 μmol scale. The yield of the product was 54.8 mg, and its estimated purity by LCMS analysis was 97.3%. Analysis condition A: Retention time=1.79 min; ESI-MS(+) m/z [M+2H]²⁺: 1125.5.

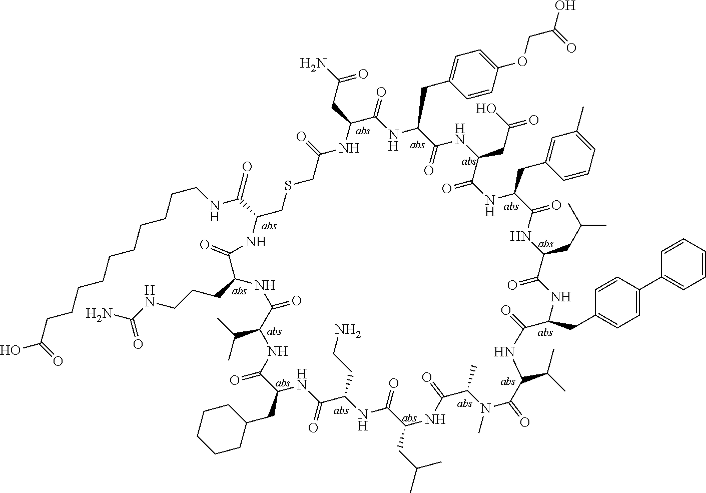

Example 2248 was prepared on a 50 μmol scale. The yield of the product was 15.8 mg, and its estimated purity by LCMS analysis was 100%. Analysis condition A: Retention time=1.86 min; ESI-MS(+) m/z [M+2H]²⁺: 1050.1.

### Preparation of Example 2249

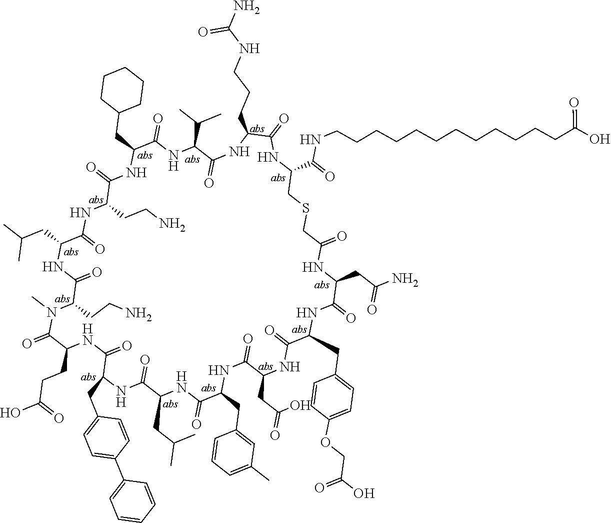 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)NCCCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)N(C)C(=O)[C@H](CCC(=O)O)NC(=O)[C@H](Cc3ccc(-c4ccccc4)cc3)NC(=O)[C@H](CC(C)C)NC2=O)c1]

Example 2249 was prepared on a 50 μmol scale. The yield of the product was 55 mg, and its estimated purity by LCMS analysis was 98.2%. Analysis condition A: Retention time=1.81 min; ESI-MS(+) m/z [M+2H]²⁺: 1065.

### Preparation of Example 2250

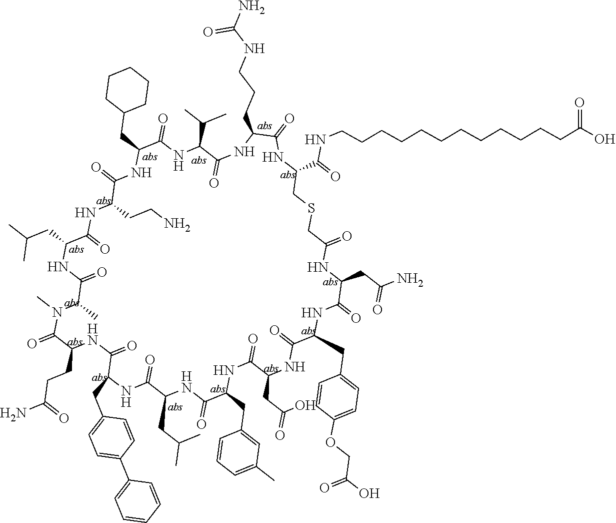 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)NCCCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](CC(C)C)NC2=O)c1]

Example 2250 was prepared on a 50 μmol scale. The yield of the product was 39.6 mg, and its estimated purity by LCMS analysis was 95.6%. Analysis condition B: Retention time=2.01 min; ESI-MS(+) m/z [M+2H]²⁺: 1065.2.

### Preparation of Example 2251

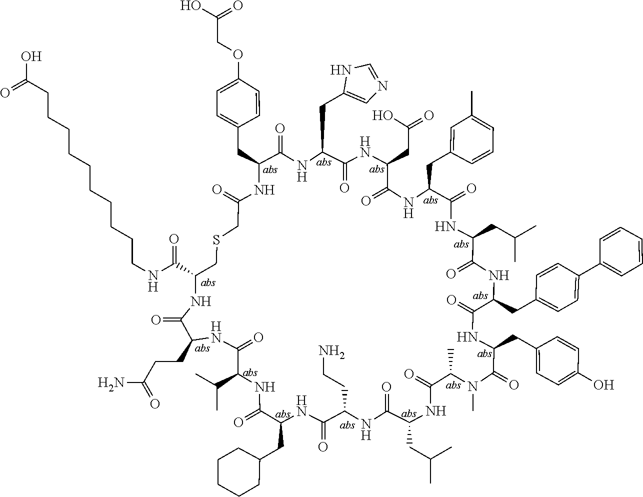 [SMILES: Cc1cccc(C[C@@H]2NC(=O)[C@H](CC(=O)O)NC(=O)[C@H](Cc3cnc[nH]3)NC(=O)[C@H](Cc3ccc(OCC(=O)O)cc3)NC(=O)CSC[C@@H](C(=O)NCCCCCCCCCCC(=O)O)NC(=O)[C@H](CCC(N)=O)NC(=O)[C@H](C(C)C)NC(=O)C[C@H](CC3CCCCC3)NC(=O)[C@H](CCN)NC(=O)C[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CC(C)C)NC2=O)c1]

Example 2251 was prepared on a 50 μmol scale. The yield of the product was 64.9 mg, and its estimated purity by LCMS analysis was 100%. Analysis condition A: Retention time=1.81 min; ESI-MS(+) m/z [M+2H]²⁺: 1079.3.

### Preparation of Example 2252

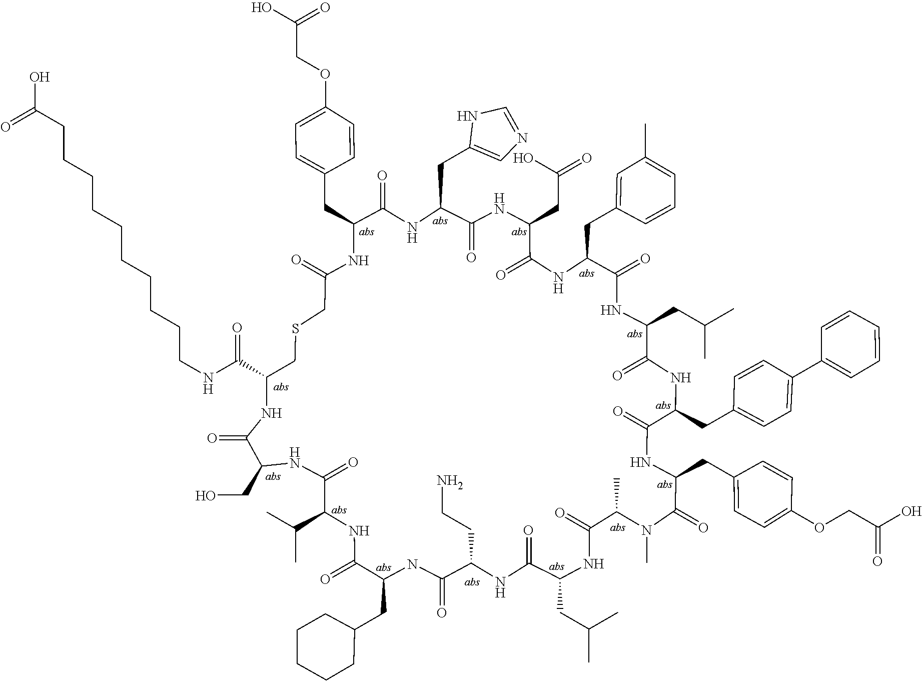

Example 2252 was prepared on a 50 μmol scale. The yield of the product was 71.6 mg, and its estimated purity by LCMS analysis was 96.2%. Analysis condition A: Retention time=1.75 min; ESI-MS(+) m/z [M+2H]²⁺: 1088.1.

### Preparation of Example 2253

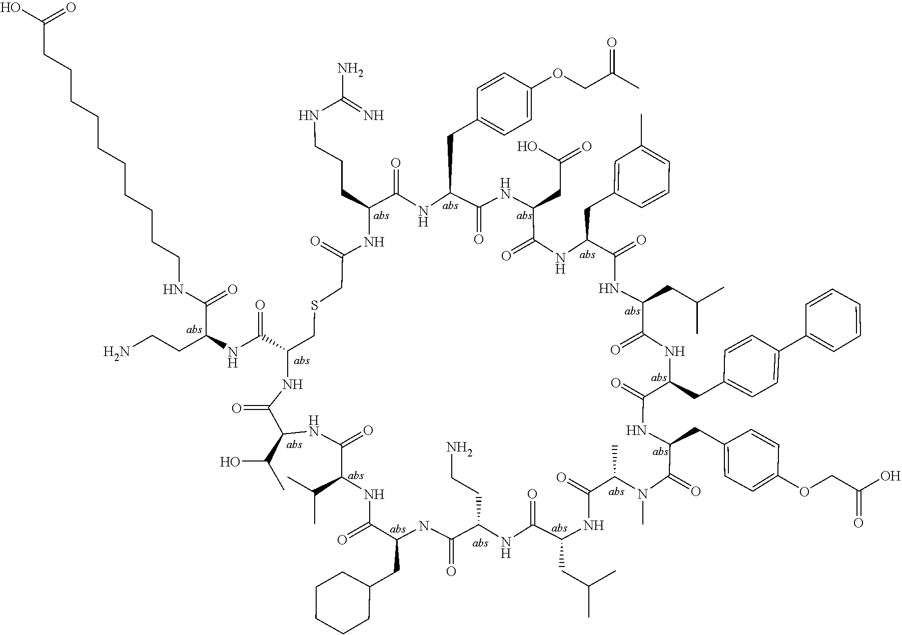

Example 2253 was prepared on a 50 μmol scale. The yield of the product was 52.6 mg, and its estimated purity by LCMS analysis was 100%. Analysis condition A: Retention time=1.75 min; ESI-MS(+) m/z [M+2H]²⁺: 1154.1.

### Preparation of Example 2254

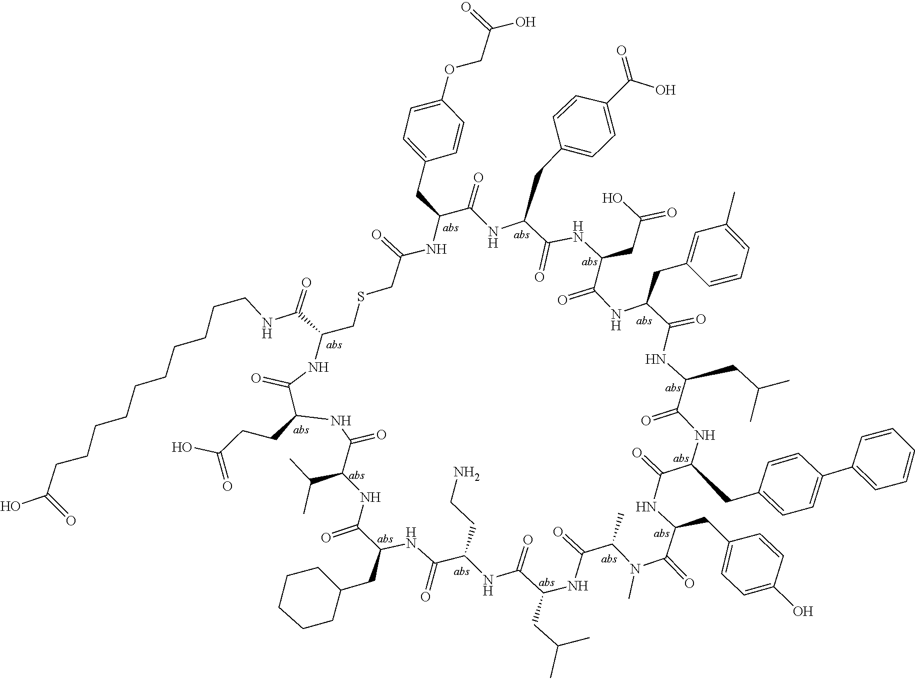

Example 2254 was prepared on a 50 μmol scale. The yield of the product was 58.7 mg, and its estimated purity by LCMS analysis was 98.8%. Analysis condition A: Retention time=1.56 min; ESI-MS(+) m/z [M+2H]²⁺: 1107.1.

### Preparation of Example 2255

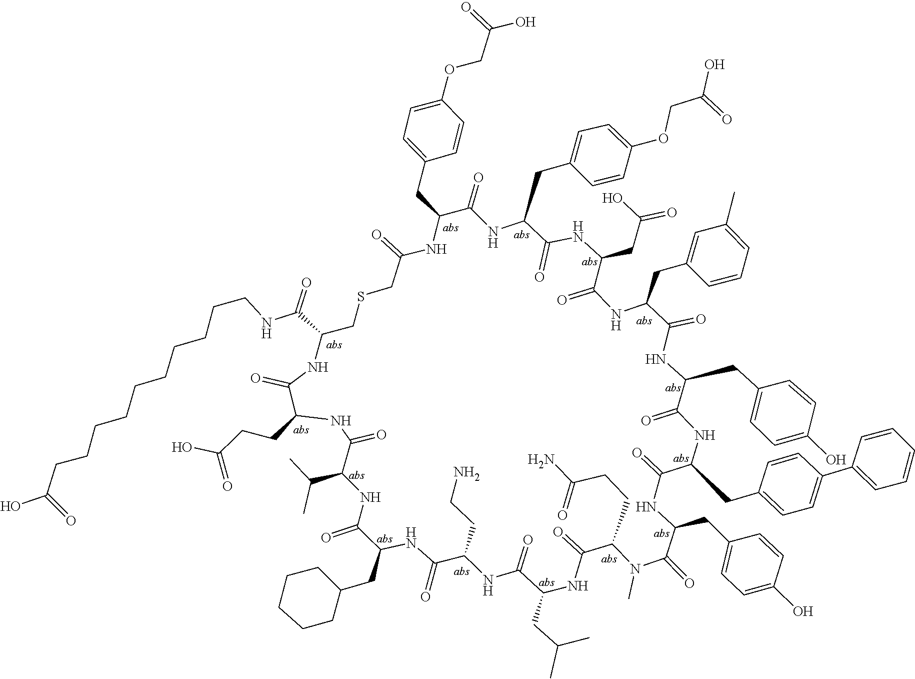

Example 2255 was prepared on a 50 μmol scale. The yield of the product was 101 mg, and its estimated purity by LCMS analysis was 98.1%. Analysis condition A: Retention time=1.39 min; ESI-MS(+) m/z [M+2H]²⁺: 1175.

### Preparation of Example 2256

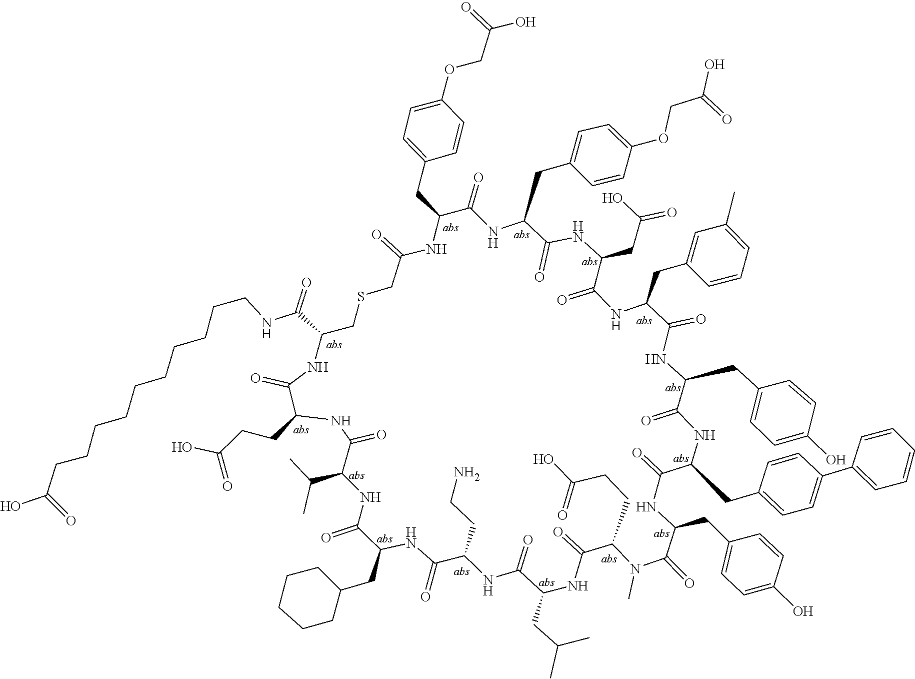

Example 2256 was prepared on a 50 μmol scale. The yield of the product was 45 mg, and its estimated purity by LCMS analysis was 100%. Analysis condition A: Retention time=1.3 min; ESI-MS(+) m/z [M+2H]²⁺: 1176.1.

### Preparation of Example 2257

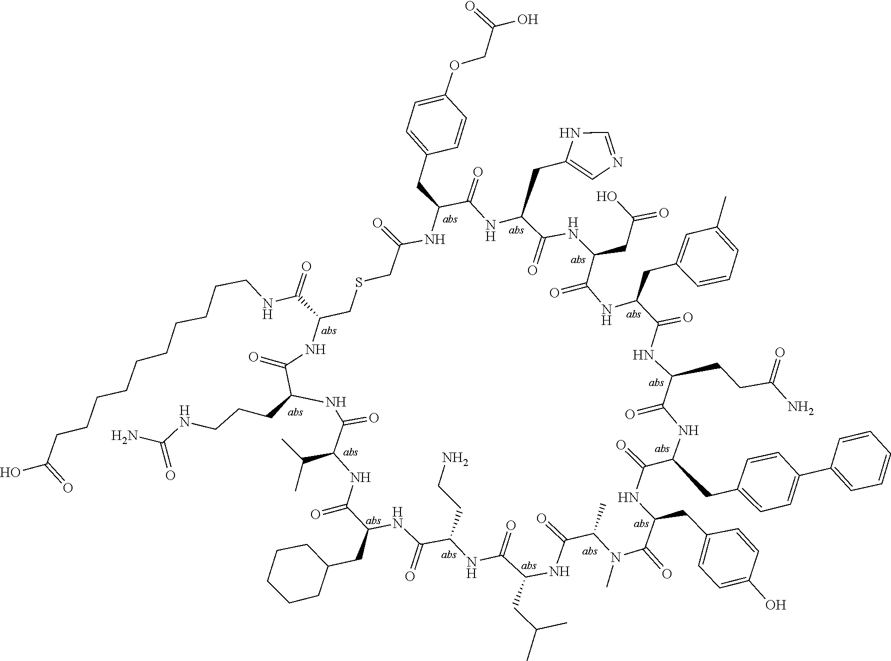 [SMILES: Cc1cccc(C[C@@H]2NC(=O)[C@H](CC(=O)O)NC(=O)[C@H](Cc3cnc[nH]3)NC(=O)[C@H](Cc3ccc(OCC(=O)O)cc3)NC(=O)CSC[C@@H](C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CCC(N)=O)NC2=O)c1]

Example 2257 was prepared on a 50 μmol scale. The yield of the product was 23.4 mg, and its estimated purity by LCMS analysis was 96.9%. Analysis condition B: Retention time=1.66 min; ESI-MS(+) m/z [M+2H]²⁺: 1101.3.

### Preparation of Example 2258

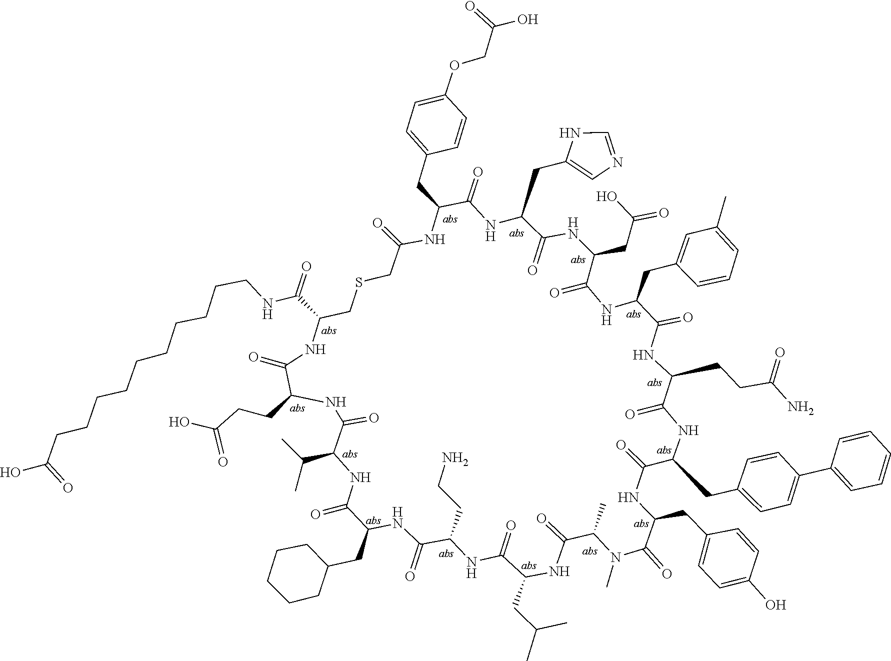 [SMILES: Cc1cccc(C[C@@H]2NC(=O)[C@H](CC(=O)O)NC(=O)[C@H](Cc3cnc[nH]3)NC(=O)[C@H](Cc3ccc(OCC(=O)O)cc3)NC(=O)CSC[C@@H](C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CCC(N)=O)NC2=O)c1]

Example 2258 was prepared on a 50 μmol scale. The yield of the product was 23.5 mg, and its estimated purity by LCMS analysis was 100%. Analysis condition A: Retention time=1.72 min; ESI-MS(+) m/z [M+2H]²⁺: 1087.3.

### Preparation of Example 2259

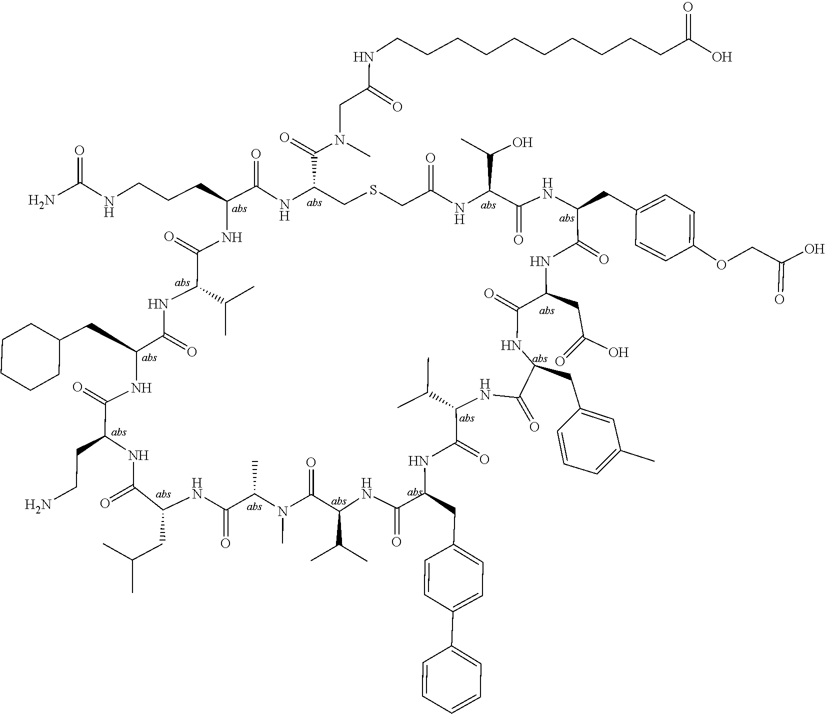

Example 2259 was prepared on a 50 μmol scale. The yield of the product was 73 mg, and its estimated purity by LCMS analysis was 95.1%. Analysis condition B: Retention time=1.93 min; ESI-MS(+) m/z [M+2H]²⁺: 1073.

### Preparation of Example 2260

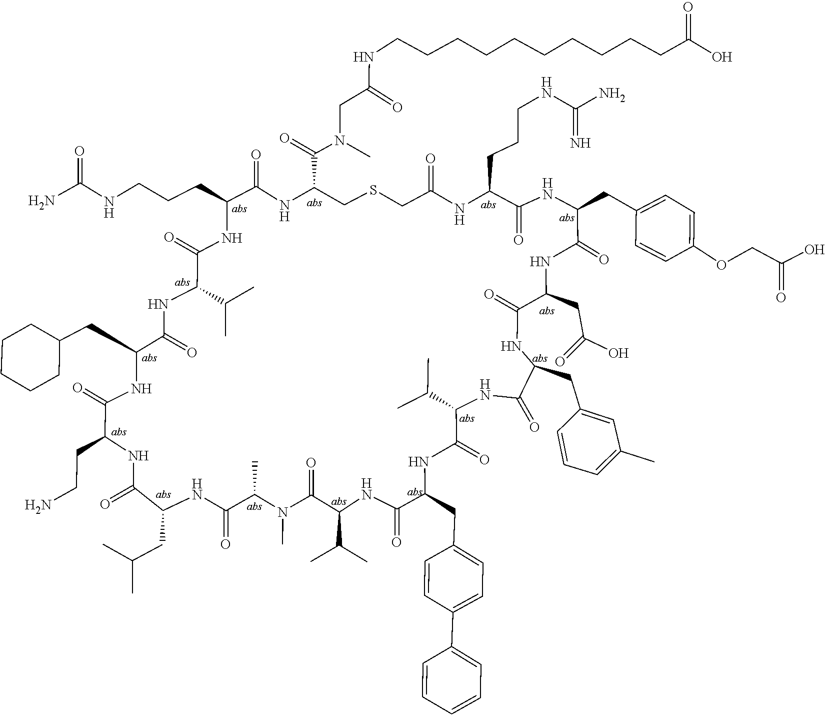

Example 2260 was prepared on a 50 μmol scale. The yield of the product was 50.4 mg, and its estimated purity by LCMS analysis was 100%. Analysis condition A: Retention time=1.73 min; ESI-MS(+) m/z [M+2H]²⁺: 1100.

### Preparation of Example 2261

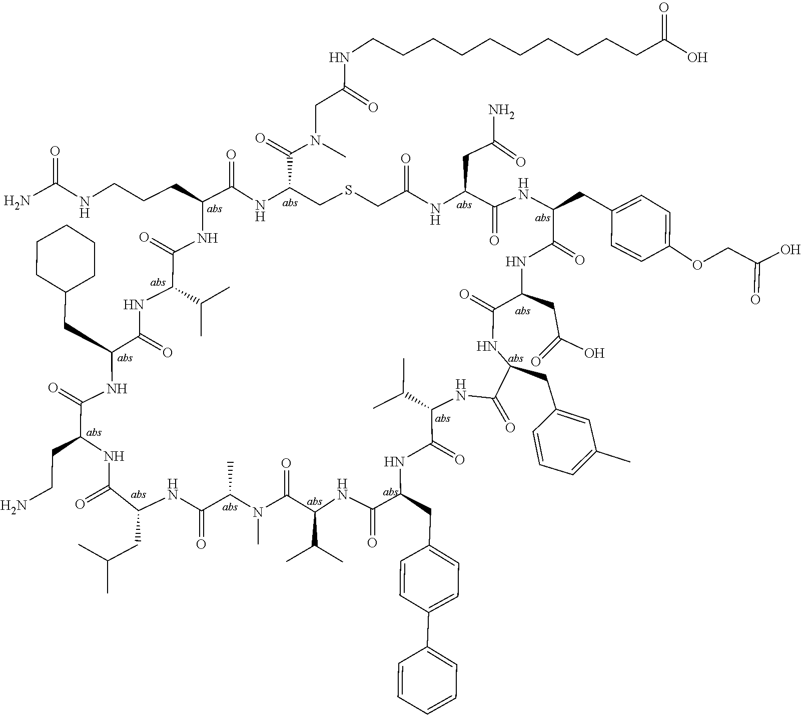

Example 2261 was prepared on a 50 μmol scale. The yield of the product was 77.6 mg, and its estimated purity by LCMS analysis was 85.7%. Analysis condition B: Retention time=1.91 min; ESI-MS(+) m/z [M+2H]²⁺: 1079.2.

### Preparation of Example 2262

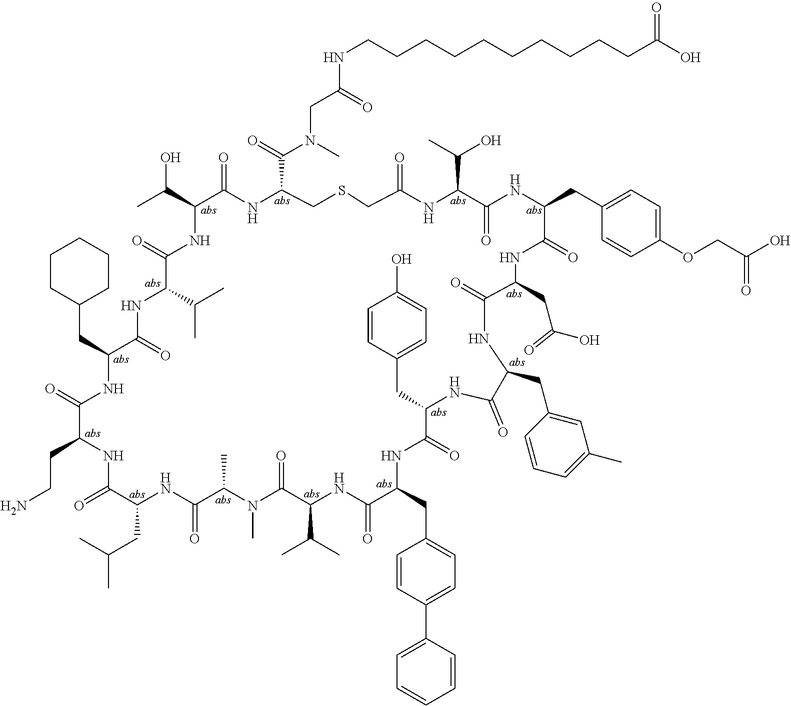

Example 2262 was prepared on a 50 μmol scale. The yield of the product was 72.4 mg, and its estimated purity by LCMS analysis was 97.2%. Analysis condition A: Retention time=1.74 min; ESI-MS(+) m/z [M+2H]²⁺: 1076.3.

### Preparation of Example 2263

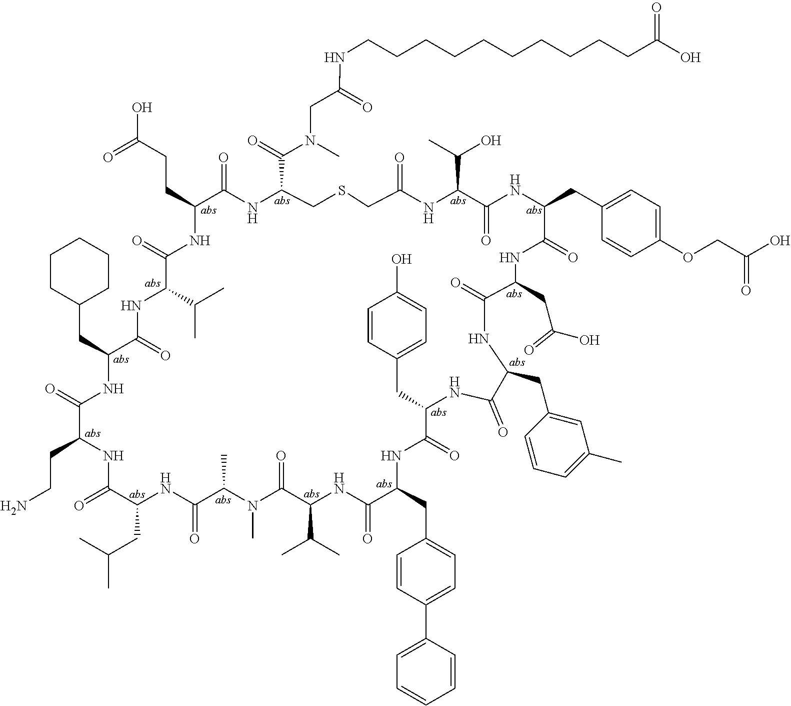

Example 2263 was prepared on a 50 μmol scale. The yield of the product was 22.8 mg, and its estimated purity by LCMS analysis was 96.1%. Analysis condition A: Retention time=1.64 min; ESI-MS(+) m/z [M+2H]²⁺: 1091.1.

### Preparation of Example 2264

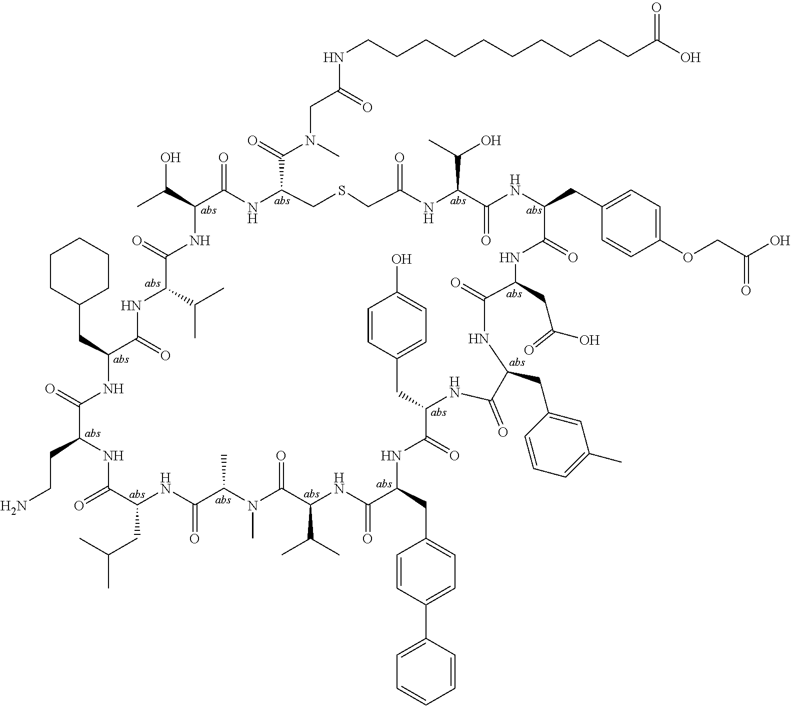

Example 2264 was prepared on a 50 μmol scale. The yield of the product was 46.5 mg, and its estimated purity by LCMS analysis was 97%. Analysis condition A: Retention time=1.73 min; ESI-MS(+) m/z [M+2H]²⁺: 1083.1.

### Preparation of Example 2265

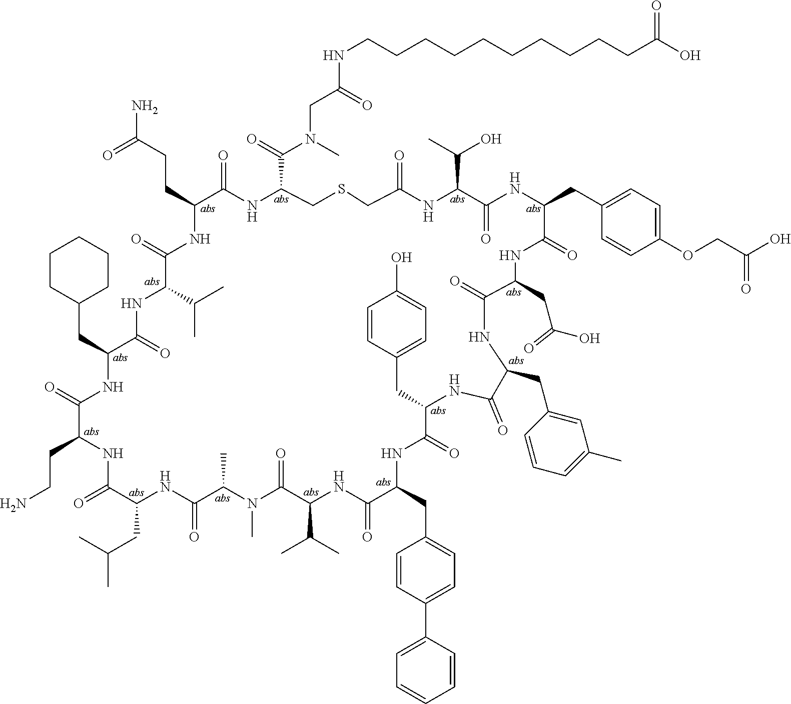 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](Cc3ccc(O)cc3)NC2=O)c1]

Example 2265 was prepared on a 50 μmol scale. The yield of the product was 5.9 mg, and its estimated purity by LCMS analysis was 99.4%. Analysis condition A: Retention time=1.63 min; ESI-MS(+) m/z [M+2H]²⁺: 1090.1.

### Preparation of Example 2266

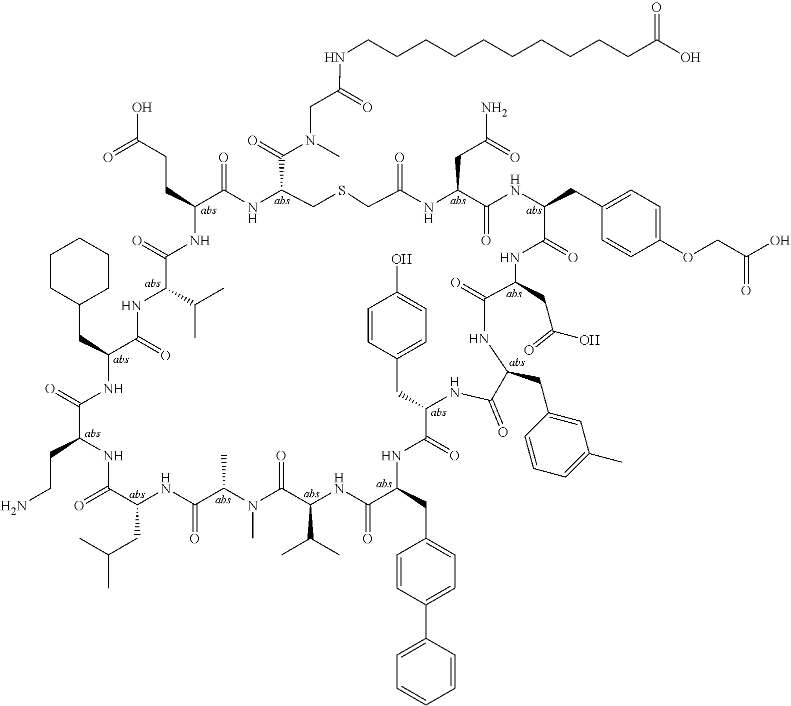

Example 2266 was prepared on a 50 μmol scale. The yield of the product was 58.1 mg, and its estimated purity by LCMS analysis was 96.3%. Analysis condition A: Retention time=1.61 min; ESI-MS(+) m/z [M+2H]²⁺: 1097.2.

### Preparation of Example 2267

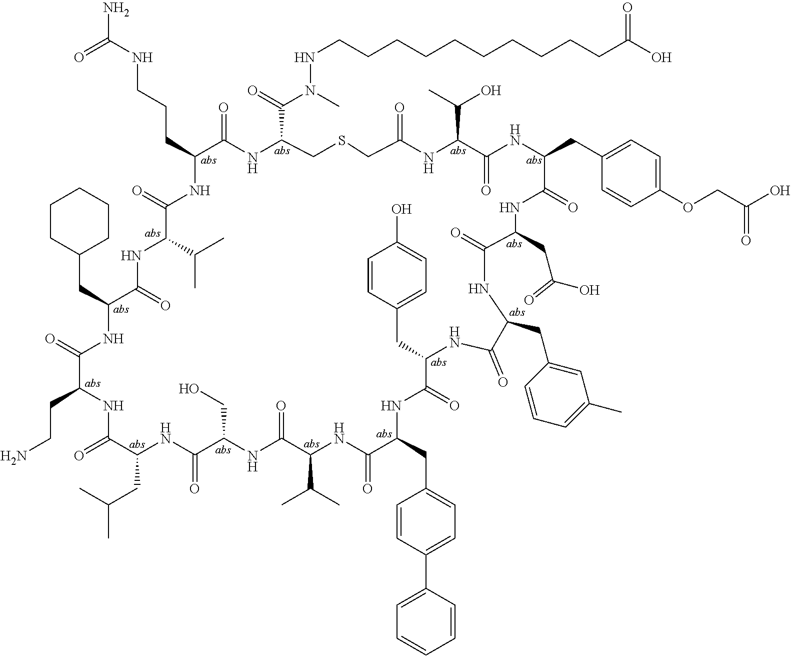

Example 2267 was prepared on a 50 μmol scale. The yield of the product was 5.7 mg, and its estimated purity by LCMS analysis was 97.3%. Analysis condition B: Retention time=1.77 min; ESI-MS(+) m/z [M+2H]²⁺: 1112.4.

### Preparation of Example 2268

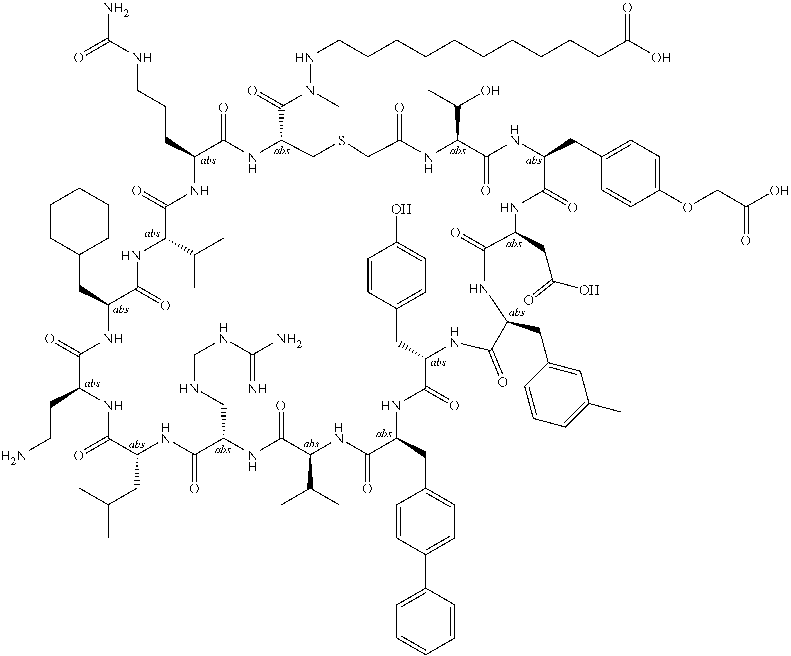

Example 2268 was prepared on a 50 μmol scale. The yield of the product was 10.3 mg, and its estimated purity by LCMS analysis was 92.8%. Analysis condition B: Retention time=1.82 min; ESI-MS(+) m/z [M+2H]²⁺: 1147.3.

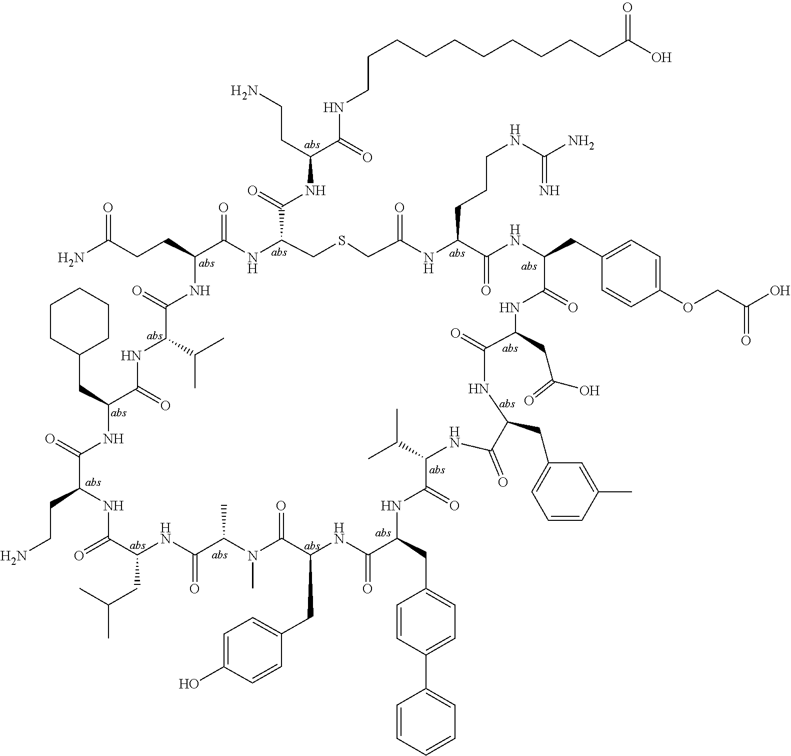

Example 2269 was prepared on a 50 μmol scale. The yield of the product was 41.4 mg, and its estimated purity by LCMS analysis was 98.6%. Analysis condition A: Retention time=1.61 min; ESI-MS(+) m/z [M+2H]²⁺: 1131.8.

### Preparation of Example 2270

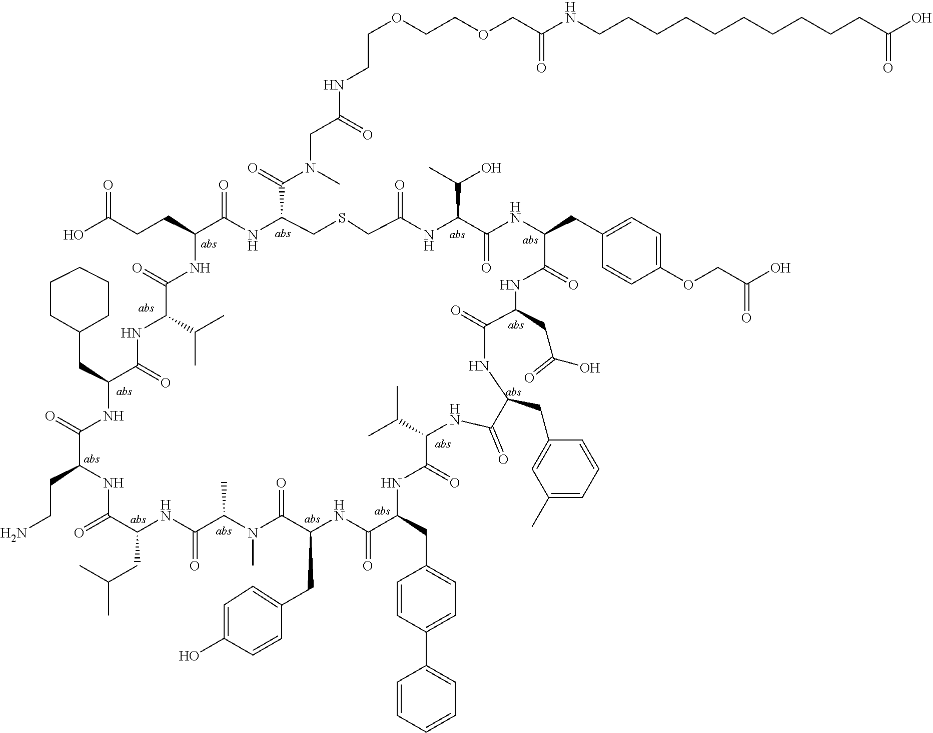

Example 2270 was prepared on a 50 μmol scale. The yield of the product was 67.9 mg, and its estimated purity by LCMS analysis was 90.1%. Analysis condition A: Retention time=1.63 min; ESI-MS(+) m/z [M+2H]²⁺: 1163.1.

### Preparation of Example 2271

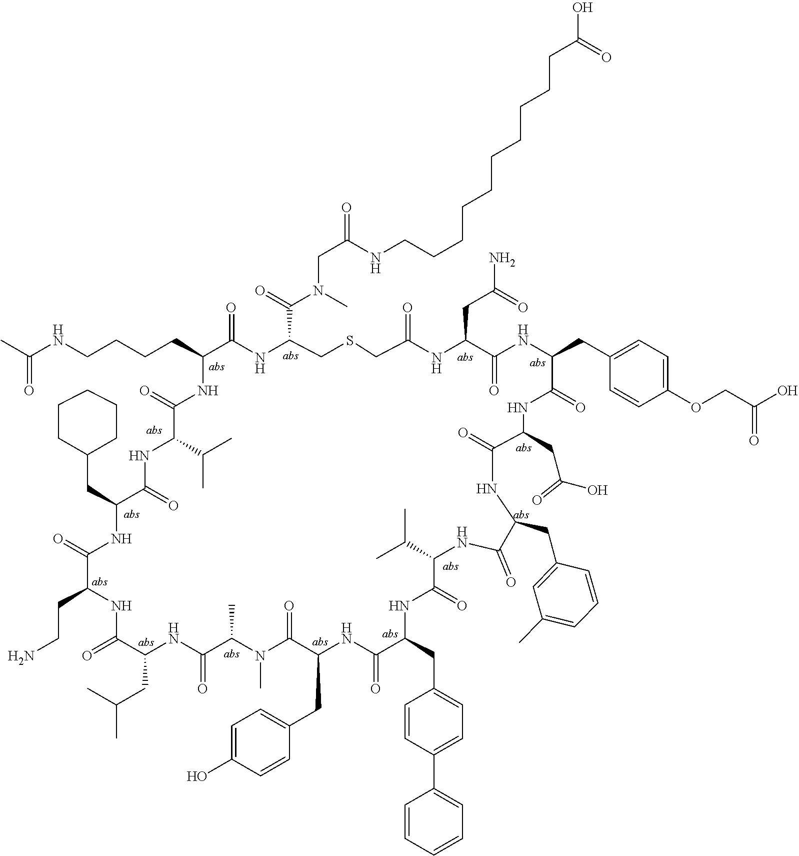

Example 2271 was prepared on a 50 μmol scale. The yield of the product was 88.1 mg, and its estimated purity by LCMS analysis was 96.8%. Analysis condition B: Retention time=1.93 min; ESI-MS(+) m/z [M+2H]²⁺: 1117.1.

### Preparation of Example 2272

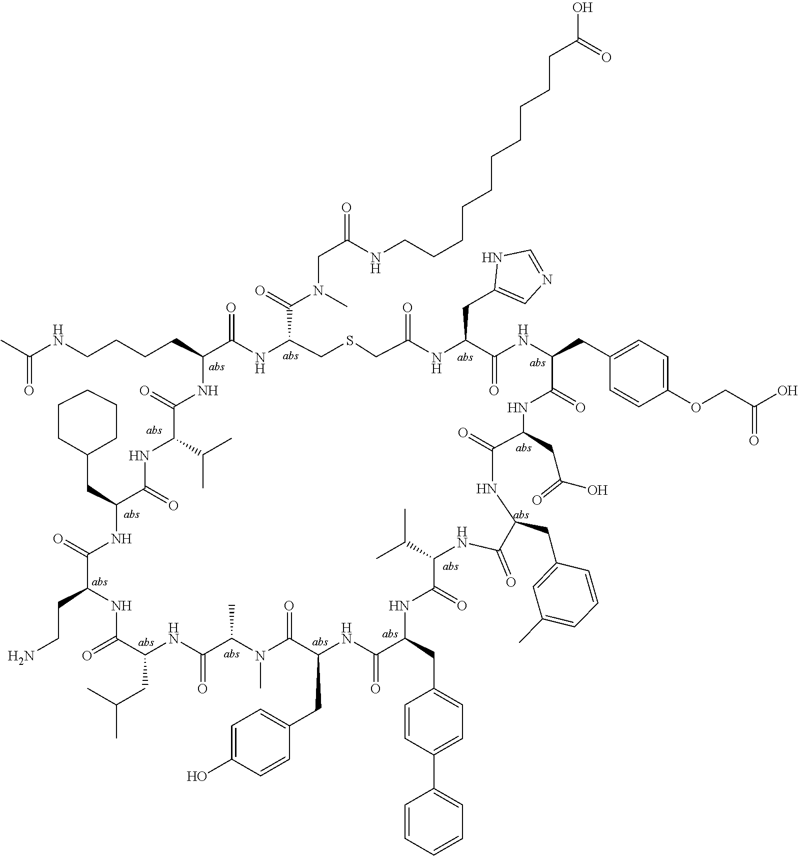

Example 2272 was prepared on a 50 μmol scale. The yield of the product was 51.2 mg, and its estimated purity by LCMS analysis was 100%. Analysis condition A: Retention time=1.64, 1.68 min; ESI-MS(+) m/z [M+2H]²⁺: 1128.87, 1128.87.

### Preparation of Example 2273

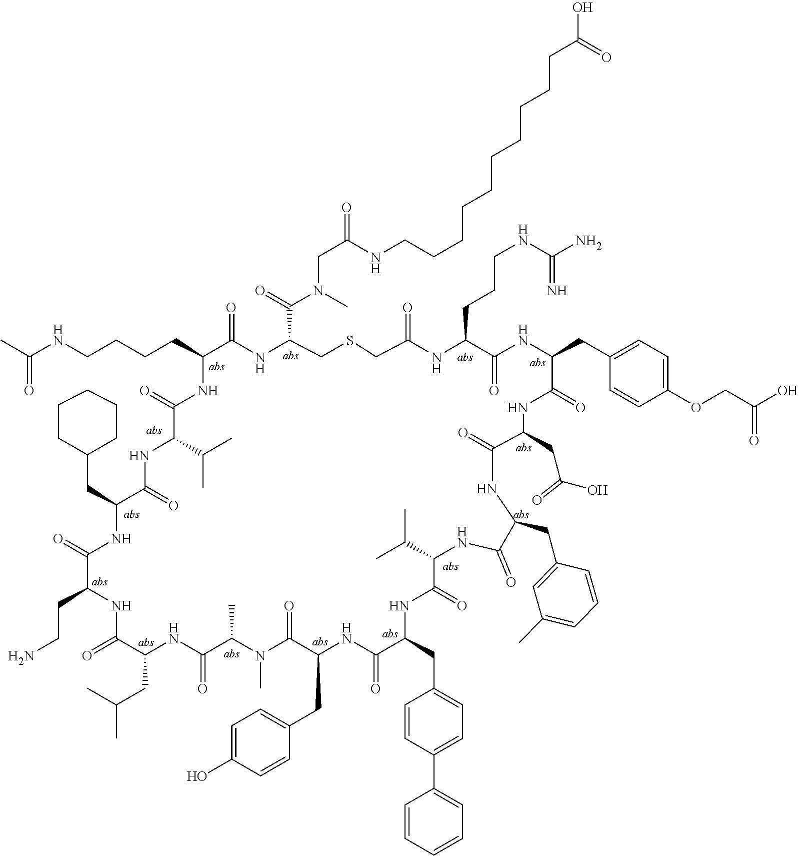

Example 2273 was prepared on a 50 μmol scale. The yield of the product was 47.9 mg, and its estimated purity by LCMS analysis was 95%. Analysis condition A: Retention time=1.72 min; ESI-MS(+) m/z [M+2H]²⁺: 1139.

### Preparation of Example 2274

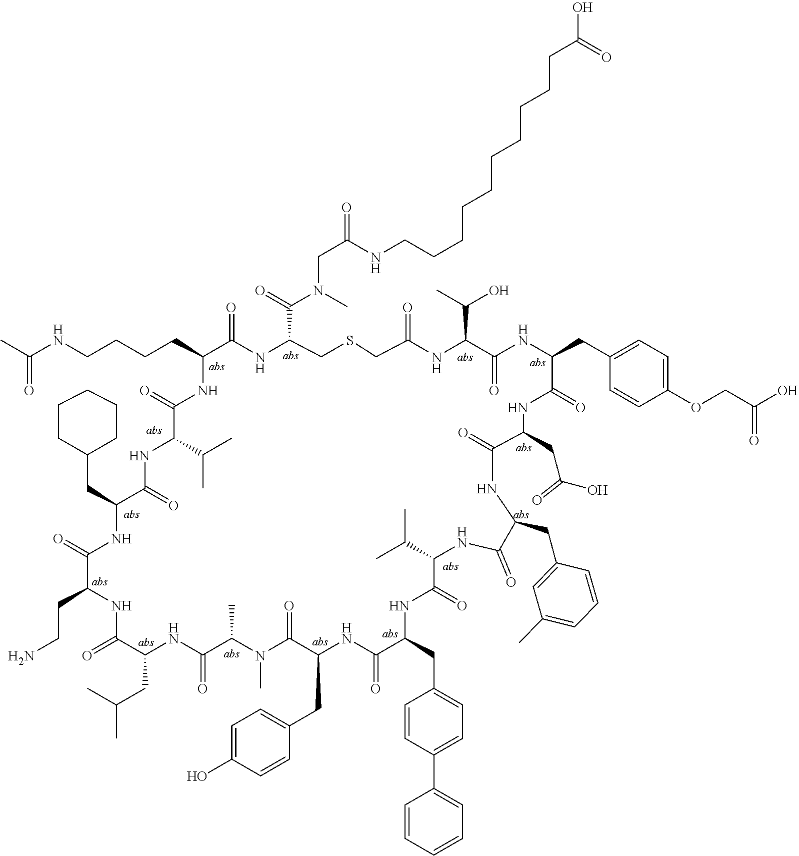

Example 2274 was prepared on a 50 μmol scale. The yield of the product was 75.7 mg, and its estimated purity by LCMS analysis was 96.1%. Analysis condition B: Retention time=1.96 min; ESI-MS(+) m/z [M+2H]²⁺: 1111.

### Preparation of Example 2275

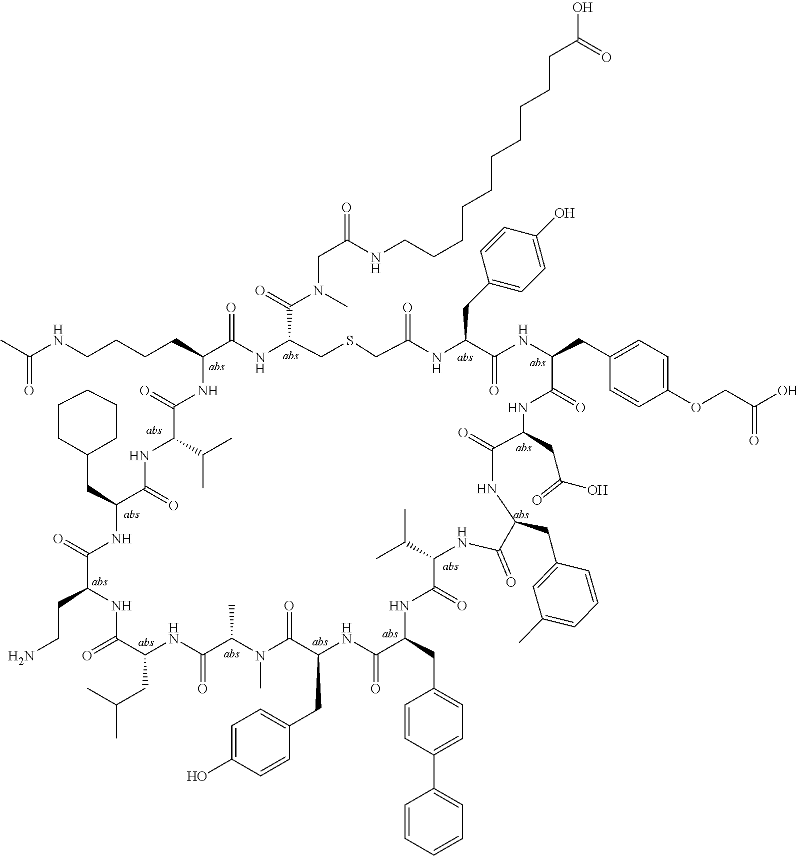

Example 2275 was prepared on a 50 μmol scale. The yield of the product was 21.2 mg, and its estimated purity by LCMS analysis was 95.5%. Analysis condition B: Retention time=1.98 min; ESI-MS(+) m/z [M+2H]²⁺: 1142.

### Preparation of Example 2276

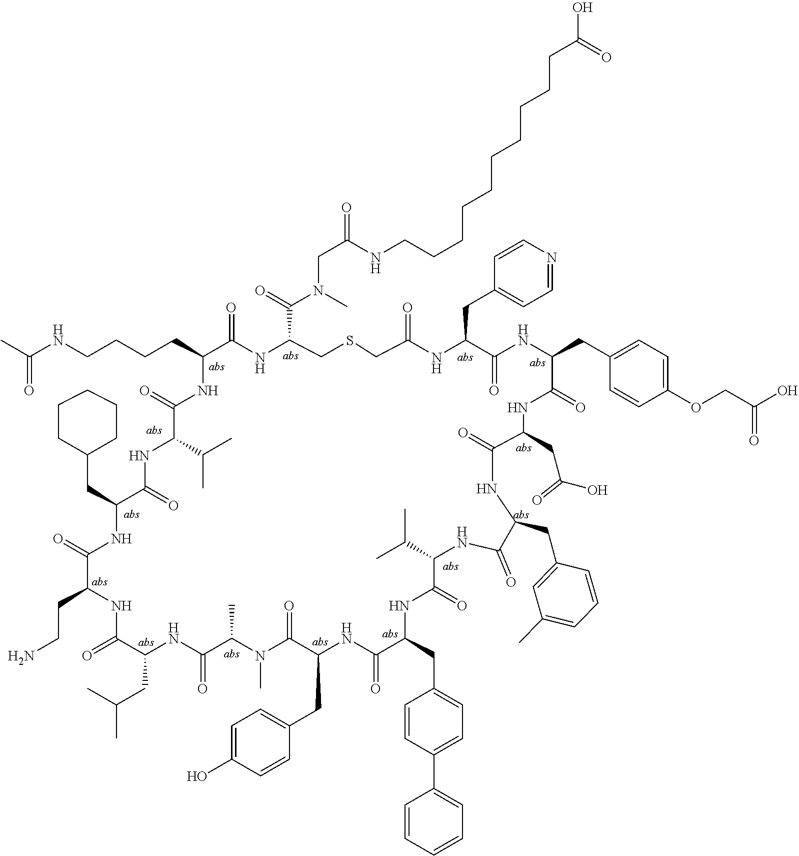

Example 2276 was prepared on a 50 μmol scale. The yield of the product was 46.2 mg, and its estimated purity by LCMS analysis was 96.5%. Analysis condition A: Retention time=1.68 min; ESI-MS(+) m/z [M+2H]²⁺: 1134.2.

### Preparation of Example 2277

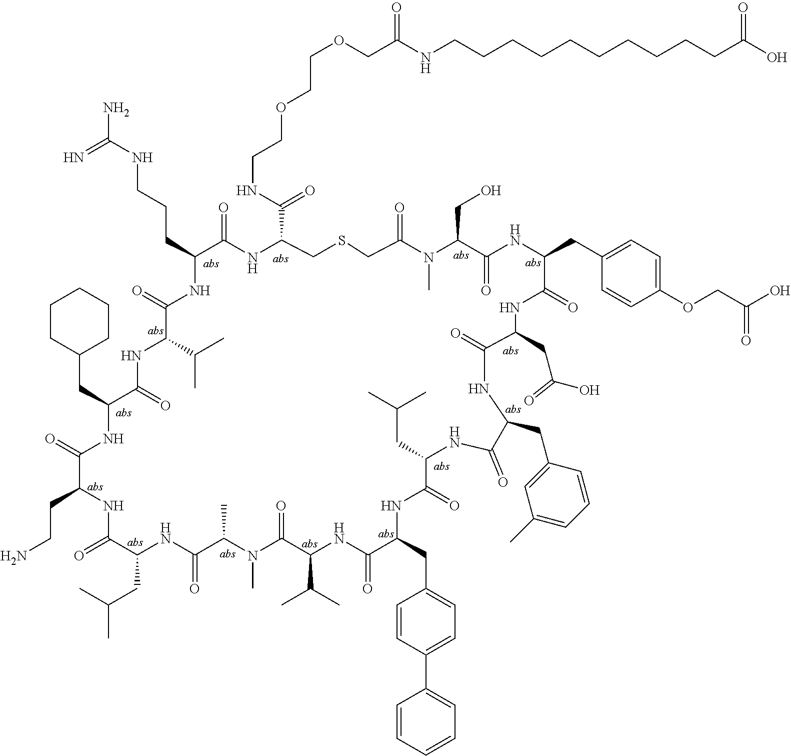

Example 2277 was prepared on a 50 μmol scale. The yield of the product was 60.4 mg, and its estimated purity by LCMS analysis was 98.1%. Analysis condition B: Retention time=1.94 min; ESI-MS(+) m/z [M+2H]²⁺: 1116.2.

### Preparation of Example 2278

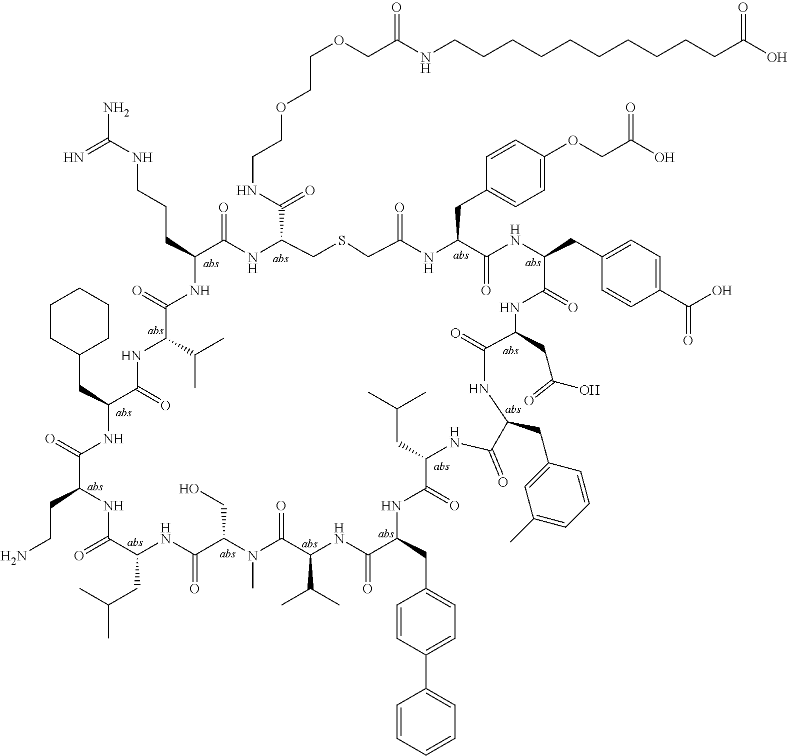

Example 2278 was prepared on a 50 μmol scale. The yield of the product was 2.8 mg, and its estimated purity by LCMS analysis was 100%. Analysis condition A: Retention time=1.62 min; ESI-MS(+) m/z [M+2H]²⁺: 1169.1.

### Preparation of Example 2279

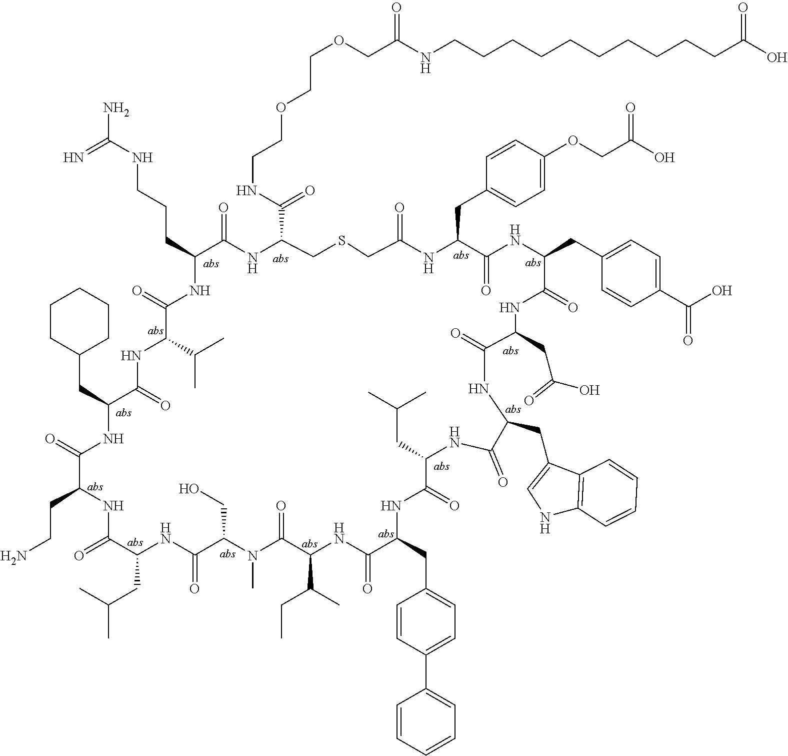

Example 2279 was prepared on a 50 μmol scale. The yield of the product was 9.2 mg, and its estimated purity by LCMS analysis was 95.1%. Analysis condition A: Retention time=1.68 min; ESI-MS(+) m/z [M+2H]²⁺: 1189.2.

### Preparation of Example 2280

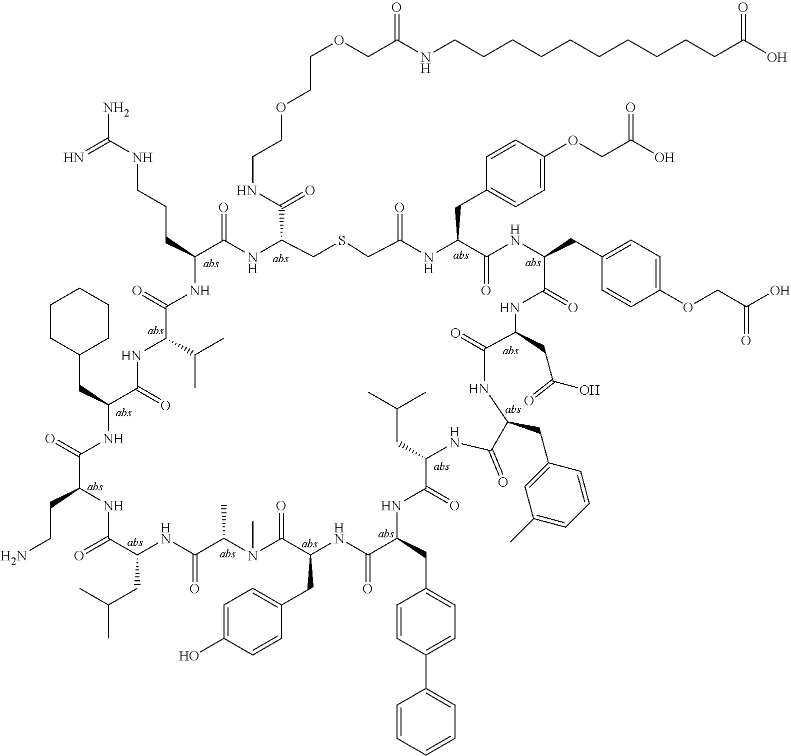

Example 2280 was prepared on a 50 μmol scale. The yield of the product was 101.3 mg, and its estimated purity by LCMS analysis was 93.4%. Analysis condition B: Retention time=1.94 min; ESI-MS(+) m/z [M+2H]²⁺: 1208.2.

### Preparation of Example 2281

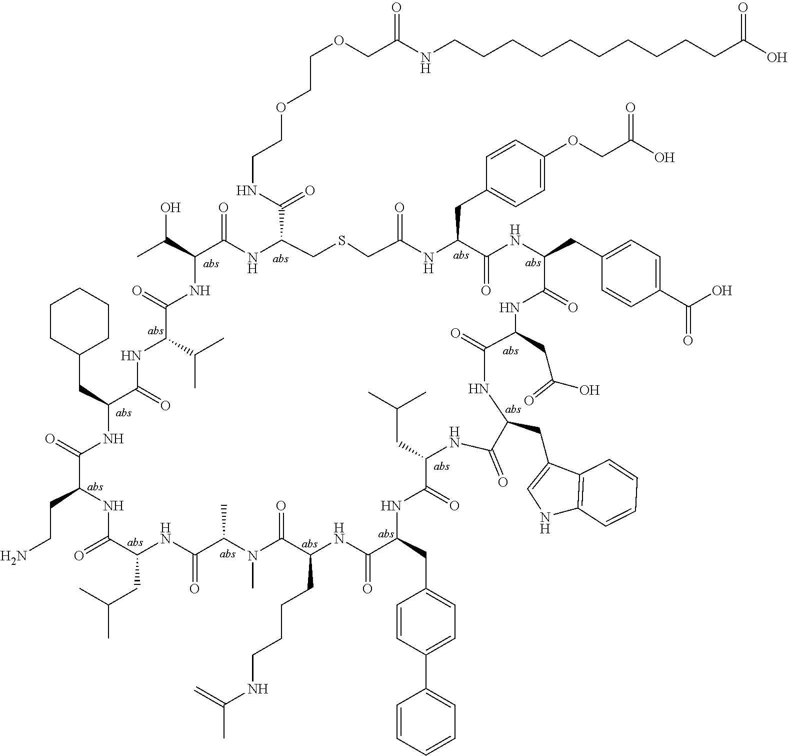

Example 2281 was prepared on a 50 μmol scale. The yield of the product was 42 mg, and its estimated purity by LCMS analysis was 87.2%. Analysis condition A: Retention time=1.69 min; ESI-MS(+) m/z [M+2H]²⁺: 1181.1.

### Preparation of Example 2282

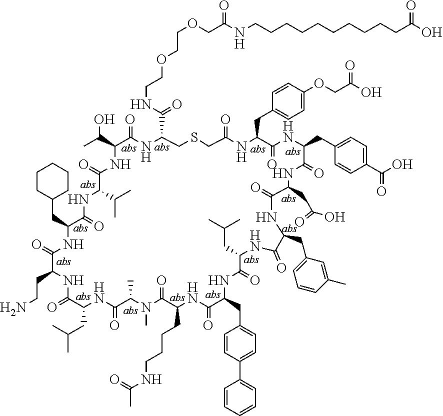 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2282 was prepared on a 50 μmol scale. The yield of the product was 105.7 mg, and its estimated purity by LCMS analysis was 92.90%. Analysis condition A: Retention time=1.88 min; ESI-MS(+) m/z [M+2H]²⁺: 1169.2.

### Preparation of Example 2283

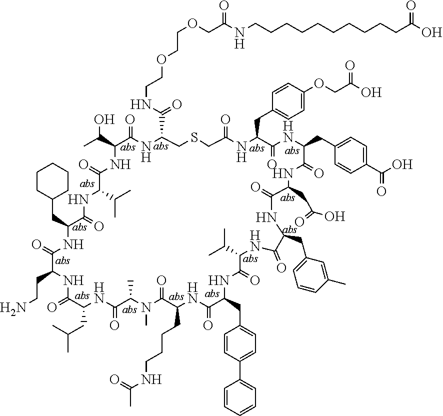 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2283 was prepared on a 50 μmol scale. The yield of the product was 65.3 mg, and its estimated purity by LCMS analysis was 99.1%. Analysis condition A: Retention time=1.67 min; ESI-MS(+) m/z [M+2H]²⁺: 1162.1.

### Preparation of Example 2284

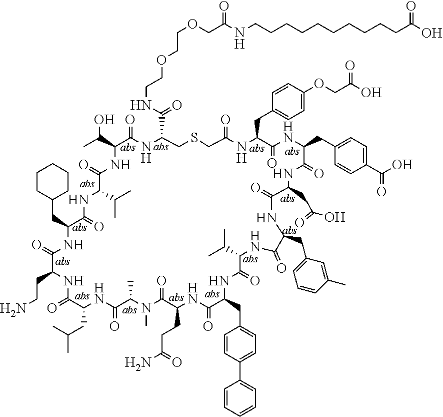 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](C(C)C)NC2=O)c1]

Example 2284 was prepared on a 50 μmol scale. The yield of the product was 44.7 mg, and its estimated purity by LCMS analysis was 100%. Analysis condition A: Retention time=1.68 min; ESI-MS(+) m/z [M+2H]²⁺: 1141.2.

### Preparation of Example 2285

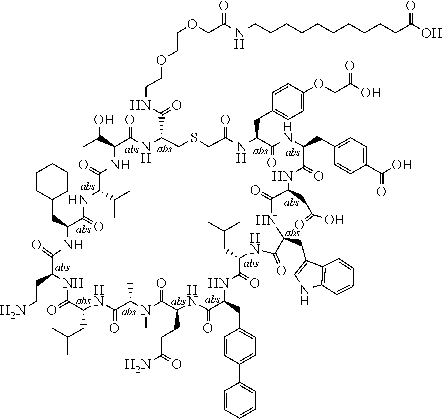 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2285 was prepared on a 50 μmol scale. The yield of the product was 68 mg, and its estimated purity by LCMS analysis was 97.4%. Analysis condition A: Retention time=1.67 min; ESI-MS(+) m/z [M+2H]²⁺:

### Preparation of Example 2286

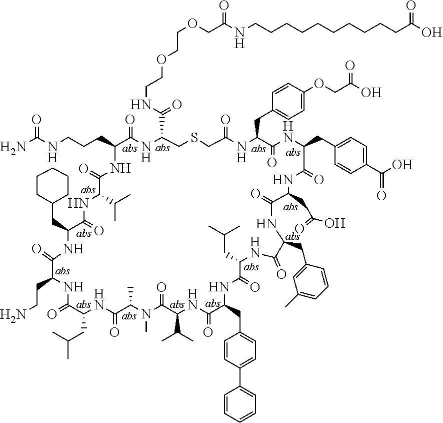 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2286 was prepared on a 50 μmol scale. The yield of the product was 22.7 mg, and its estimated purity by LCMS analysis was 97.2%. Analysis condition A: Retention time=1.68 min; ESI-MS(+) m/z [M+2H]²⁺: 1161.1.

### Preparation of Example 2287

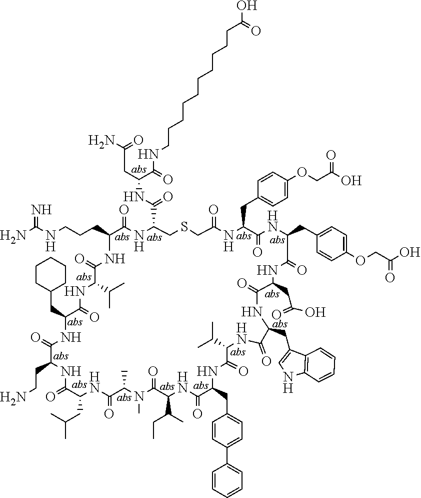 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CC(N)=O)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2287 was prepared on a 50 μmol scale. The yield of the product was 50 mg, and its estimated purity by LCMS analysis was 99%. Analysis condition B: Retention time=1.84 min; ESI-MS(+) m/z [M+3H]⁺: 782.3.

### Preparation of Example 2288

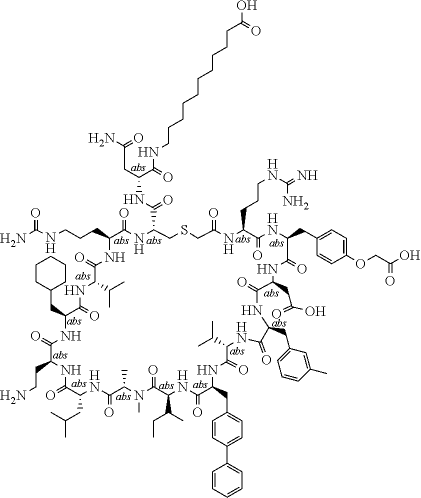 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2288 was prepared on a 50 μmol scale. The yield of the product was 30 mg, and its estimated purity by LCMS analysis was 96.6%. Analysis condition A: Retention time=1.7 min; ESI-MS(+) m/z [M+2H]²⁺: 1128.2.

### Preparation of Example 2289

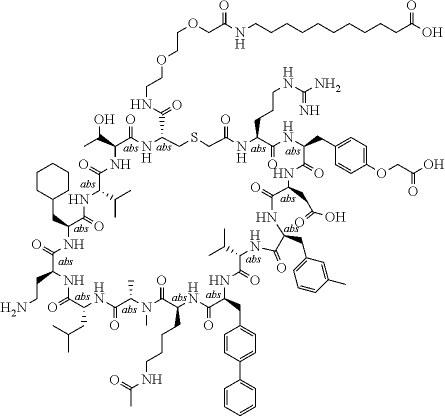 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2289 was prepared on a 50 μmol scale. The yield of the product was 98 mg, and its estimated purity by LCMS analysis was 98.5%. Analysis condition A: Retention time=1.74 min; ESI-MS(+) m/z [M+2H]²⁺: 1144.0.

### Preparation of Example 2290

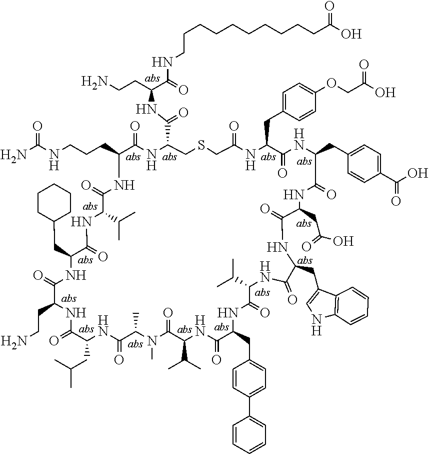 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Example 2290 was prepared on a 50 μmol scale. The yield of the product was 11.6 mg, and its estimated purity by LCMS analysis was 93.6%. Analysis condition B: Retention time=1.76 min; ESI-MS(+) m/z [M+2H]²⁺: 1145.

### Preparation of Example 2291

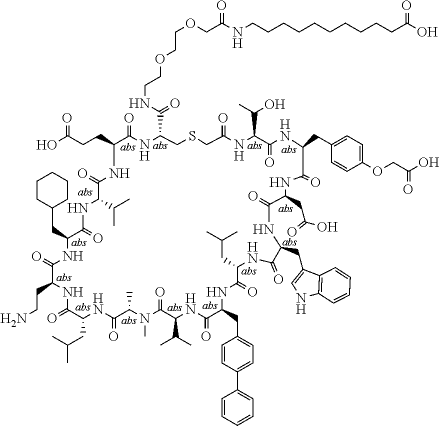 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2291 was prepared on a 50 μmol scale. The yield of the product was 42.3 mg, and its estimated purity by LCMS analysis was 100%. Analysis condition A: Retention time=1.65 min; ESI-MS(+) m/z [M+2H]²⁺: 1115.1.

### Preparation of Example 2292

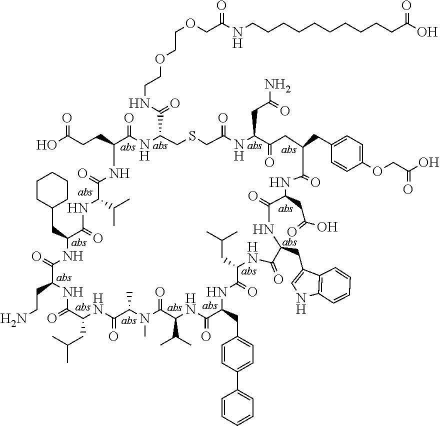 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)CC(=O)[C@H](CC(N)=O)NC(=O)CSC[C@@H](C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2292 was prepared on a 50 μmol scale. The yield of the product was 64 mg, and its estimated purity by LCMS analysis was 99%. Analysis condition B: Retention time=1.96 min; ESI-MS(+) m/z [M+2H]²⁺: 1121.2.

### Preparation of Example 2293

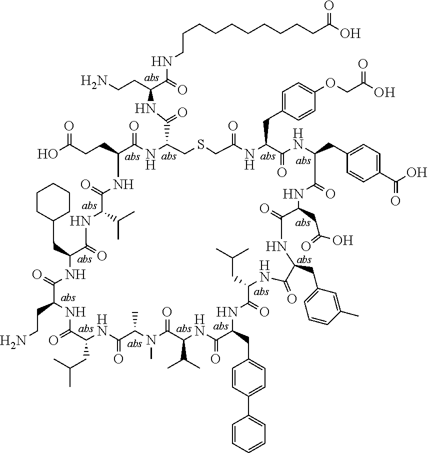 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2293 was prepared on a 50 μmol scale. The yield of the product was 37.1 mg, and its estimated purity by LCMS analysis was 84.9%. Analysis condition B: Retention time=1.92 min; ESI-MS(+) m/z [M+2H]²⁺: 1125.

### Preparation of Example 2294

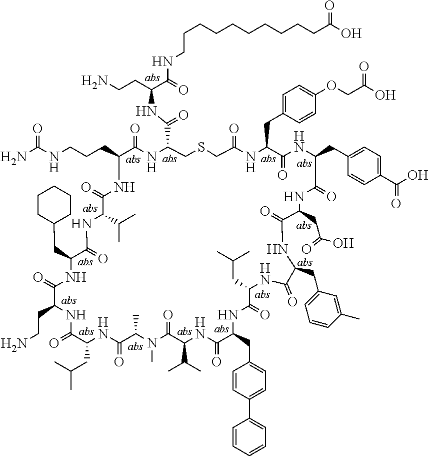 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2294 was prepared on a 50 μmol scale. The yield of the product was 25.1 mg, and its estimated purity by LCMS analysis was 96.7%. Analysis condition B: Retention time=1.92 min; ESI-MS(+) m/z [M+2H]²⁺: 1139.1.

### Preparation of Example 2295

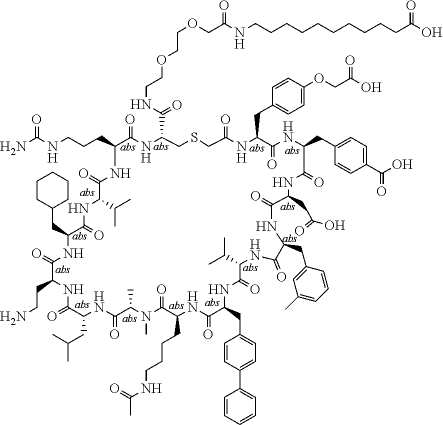 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2295 was prepared on a 50 μmol scale. The yield of the product was 46.4 mg, and its estimated purity by LCMS analysis was 97.7%. Analysis condition A: Retention time=1.53 min; ESI-MS(+) m/z [M+2H]²⁺: 1190.2.

### Preparation of Example 2296

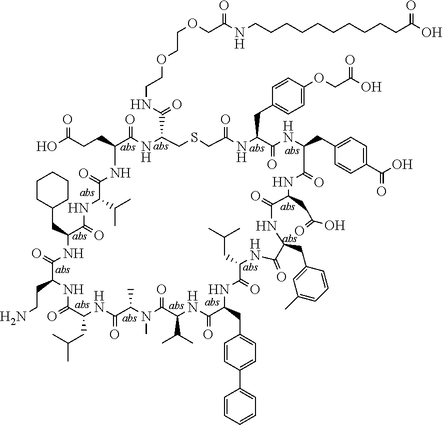 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2296 was prepared on a 50 μmol scale. The yield of the product was 35.7 mg, and its estimated purity by LCMS analysis was 97.1%. Analysis condition A: Retention time=1.61 min; ESI-MS(+) m/z [M+2H]²⁺: 1148.

### Preparation of Example 2297

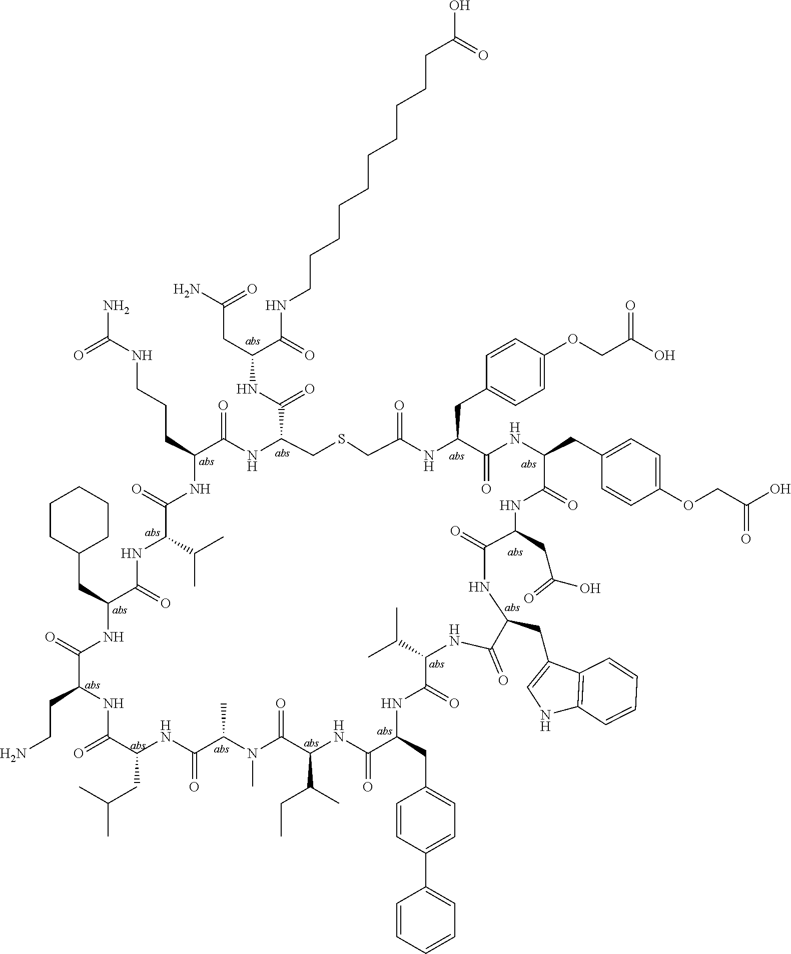 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CC(N)=O)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2297 was prepared on a 50 μmol scale. The yield of the product was 43.3 mg, and its estimated purity by LCMS analysis was 100%. Analysis condition A: Retention time=1.53 min; ESI-MS(+) m/z [M+2H]²⁺: 1173.2.

### Preparation of Example 2298

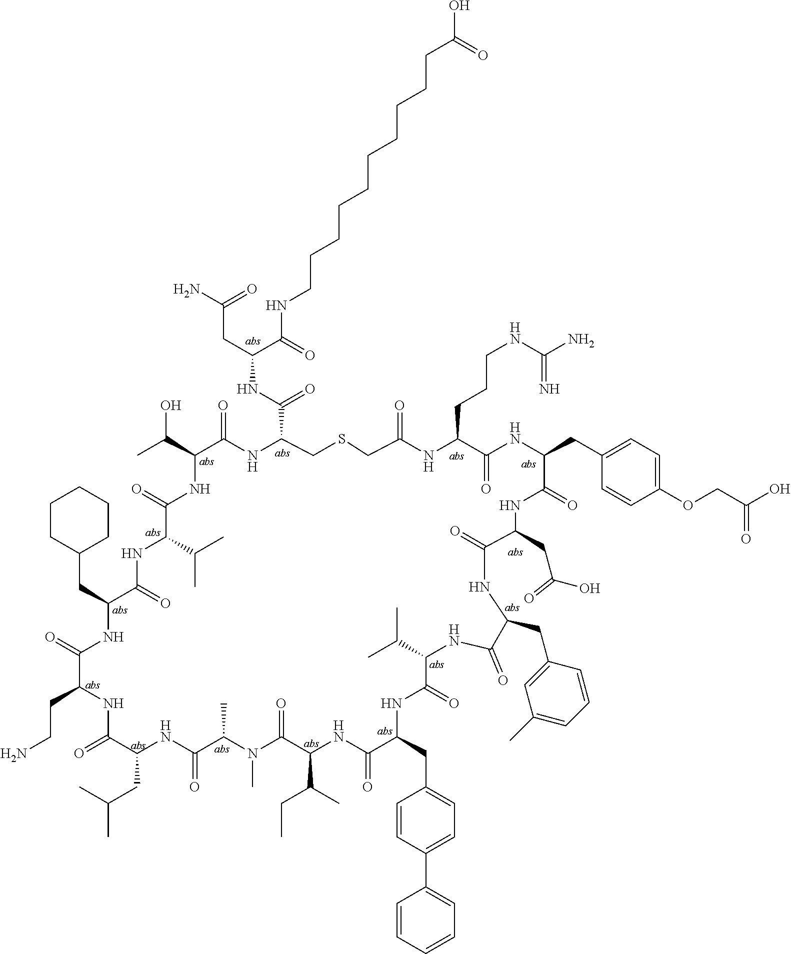 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2298 was prepared on a 50 μmol scale. The yield of the product was 47.6 mg, and its estimated purity by LCMS analysis was 100%. Analysis condition A: Retention time=1.76 min; ESI-MS(+) m/z [M+2H]²⁺: 1100.3.

### Preparation of Example 2299

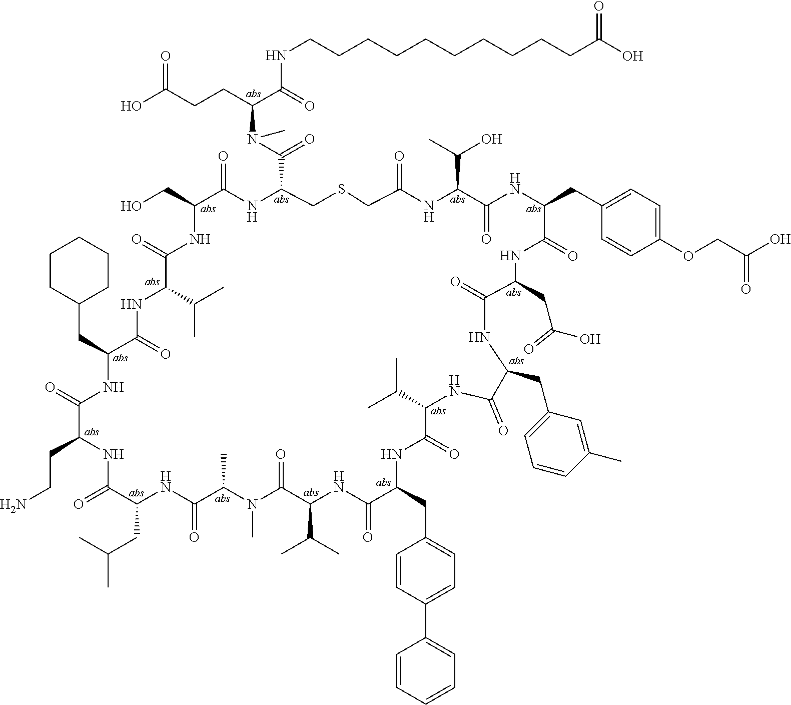 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CCC(=O)O)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2299 was prepared on a 50 μmol scale. The yield of the product was 31.9 mg, and its estimated purity by LCMS analysis was 100%. Analysis condition A: Retention time=1.59 min; ESI-MS(+) m/z [M+2H]²⁺: 1073.2.

### Preparation of Example 2300

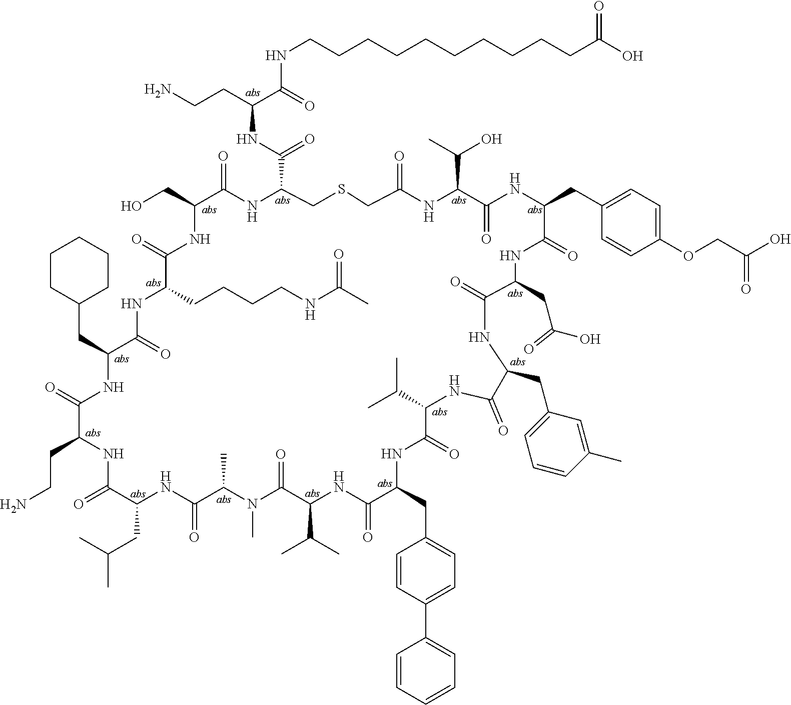 [SMILES: CC(=O)NCCCC[C@@H]1NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC1=O]

Example 2300 was prepared on a 50 μmol scale. The yield of the product was 27.7 mg, and its estimated purity by LCMS analysis was 96.4%. Analysis condition B: Retention time=1.69 min; ESI-MS(+) m/z [M+2H]²⁺: 1087.9.

### Preparation of Example 2301

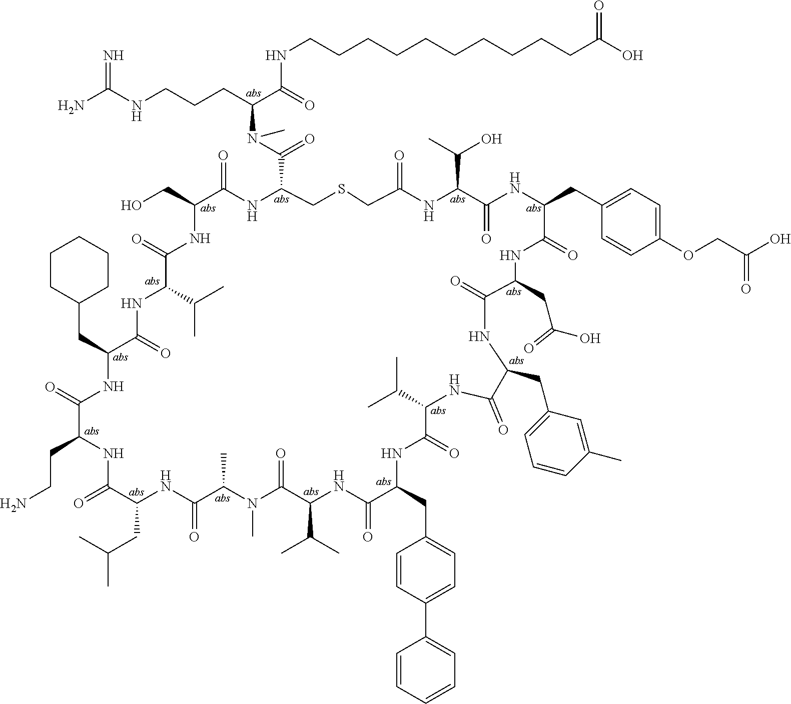 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CCCNC(=N)N)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2301 was prepared on a 50 μmol scale. The yield of the product was 22.7 mg, and its estimated purity by LCMS analysis was 96.5%. Analysis condition B: Retention time=2.55 min; ESI-MS(+) m/z [M+2H]²⁺: 1087.

### Preparation of Example 2302

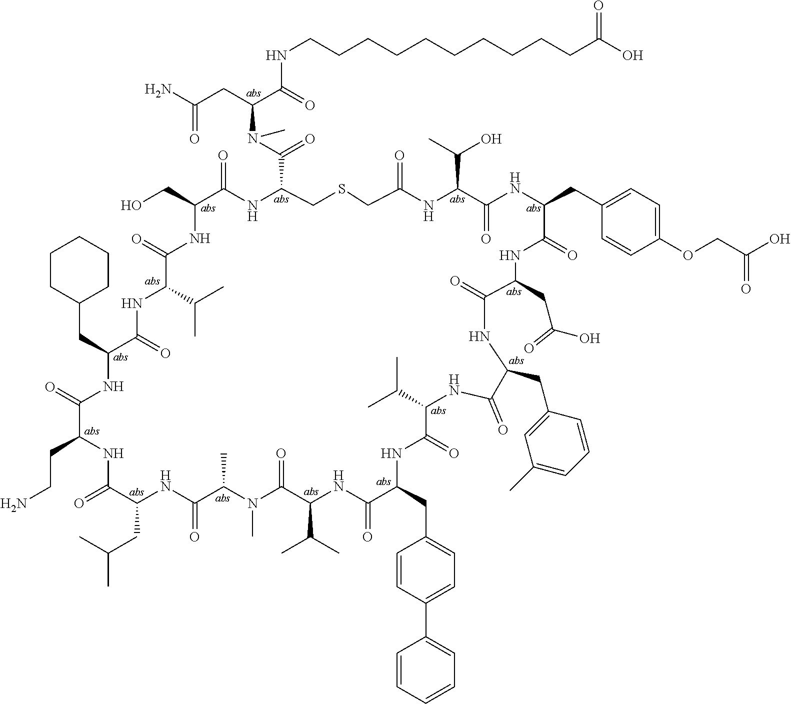 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CC(N)=O)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2302 was prepared on a 50 μmol scale. The yield of the product was 17.8 mg, and its estimated purity by LCMS analysis was 98.5%. Analysis condition B: Retention time=1.92 min; ESI-MS(+) m/z [M+2H]²⁺: 1066.

### Preparation of Example 2303

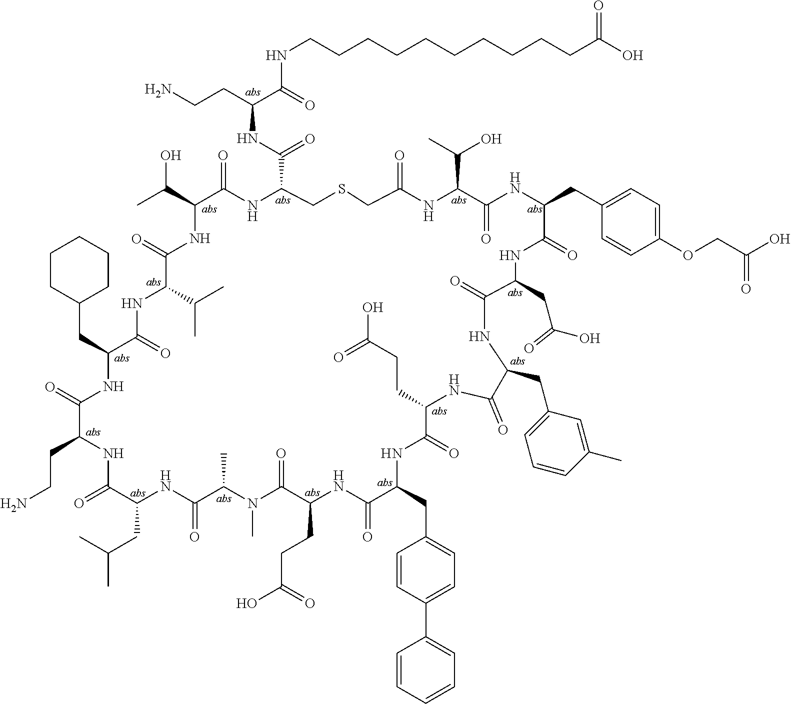 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(=O)O)NC(=O)[C@H](Cc3ccc(-c4ccccc4)cc3)NC(=O)[C@H](CCC(=O)O)NC2=O)c1]

Example 2303 was prepared on a 50 μmol scale. The yield of the product was 4 mg, and its estimated purity by LCMS analysis was 96.7%. Analysis condition A: Retention time=1.6 min; ESI-MS(+) m/z [M+2H]²⁺: 1088.9.

### Preparation of Example 2304

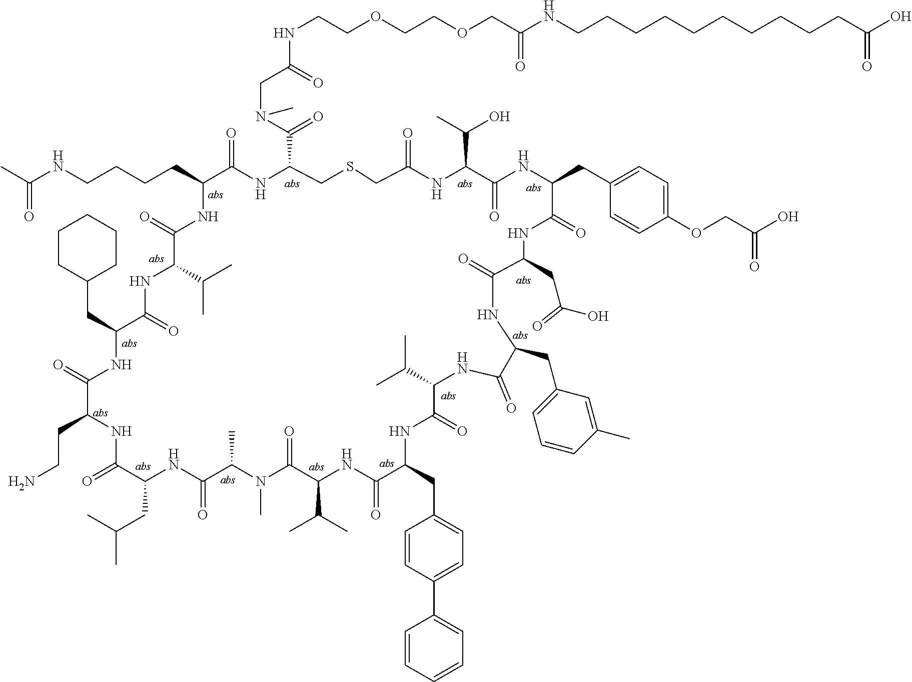 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)CC(=O)NCCOCCOCC(=O)NCCCCCCCCCCC(=O)O)NC1=O]

Example 2304 was prepared on a 50 μmol scale. The yield of the product was 49.1 mg, and its estimated purity by LCMS analysis was 97%. Analysis condition B: Retention time=2.01 min; ESI-MS(+) m/z [M+2H]²⁺: 1152.

### Preparation of Example 2305

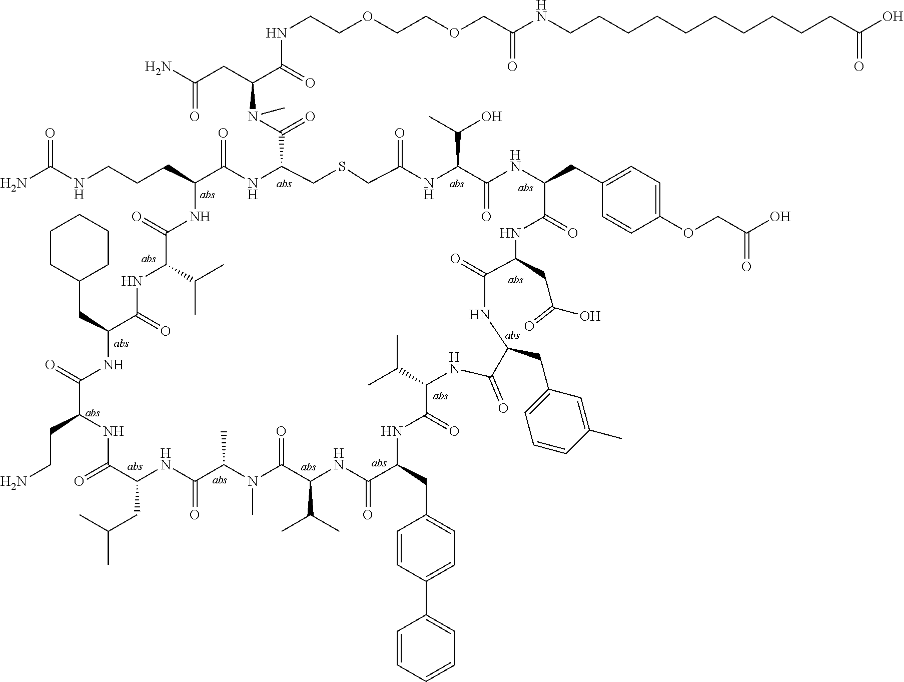 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CC(N)=O)C(=O)NCCOCCOCC(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2305 was prepared on a 50 μmol scale. The yield of the product was 11.2 mg, and its estimated purity by LCMS analysis was 98.1%. Analysis condition A: Retention time=1.65 min; ESI-MS(+) m/z [M+2H]²⁺: 1174.2.

### Preparation of Example 2306

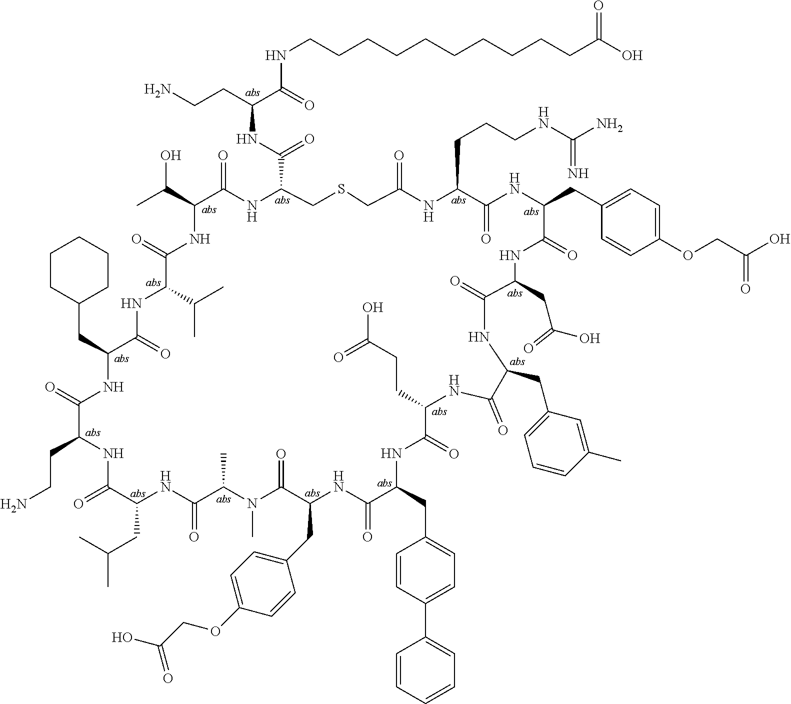 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCC(=O)O)NC2=O)c1]

Example 2306 was prepared on a 50 μmol scale. The yield of the product was 38.4 mg, and its estimated purity by LCMS analysis was 98.3%. Analysis condition B: Retention time=1.71 min; ESI-MS(+) m/z [M+3H]³⁺: 775.

### Preparation of Example 2307

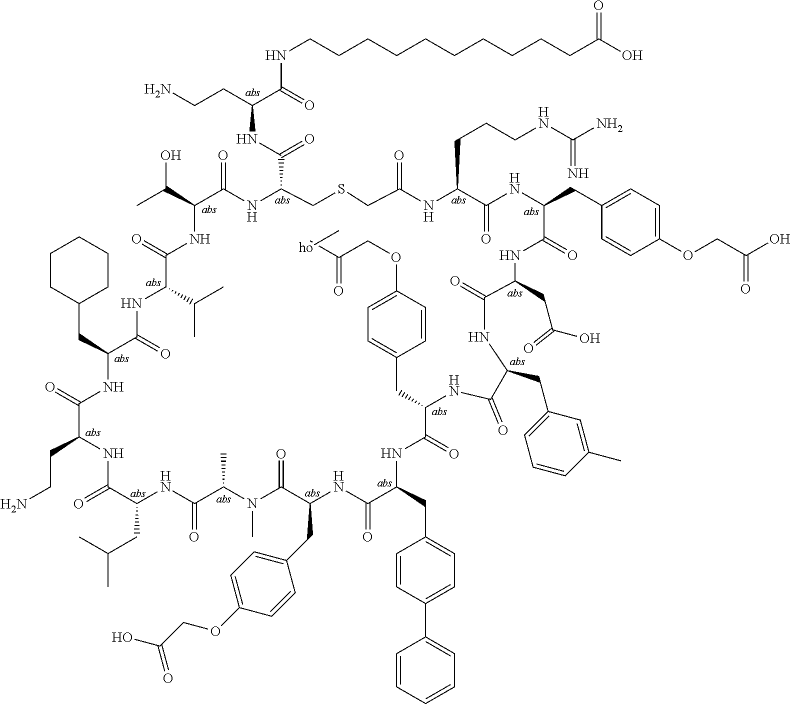

Example 2307 was prepared on a 50 μmol scale. The yield of the product was 72.5 mg, and its estimated purity by LCMS analysis was 97.5%. Analysis condition A: Retention time=1.55 min; ESI-MS(+) m/z [M+3H]³⁺: 806.1.

### Preparation of Example 2308

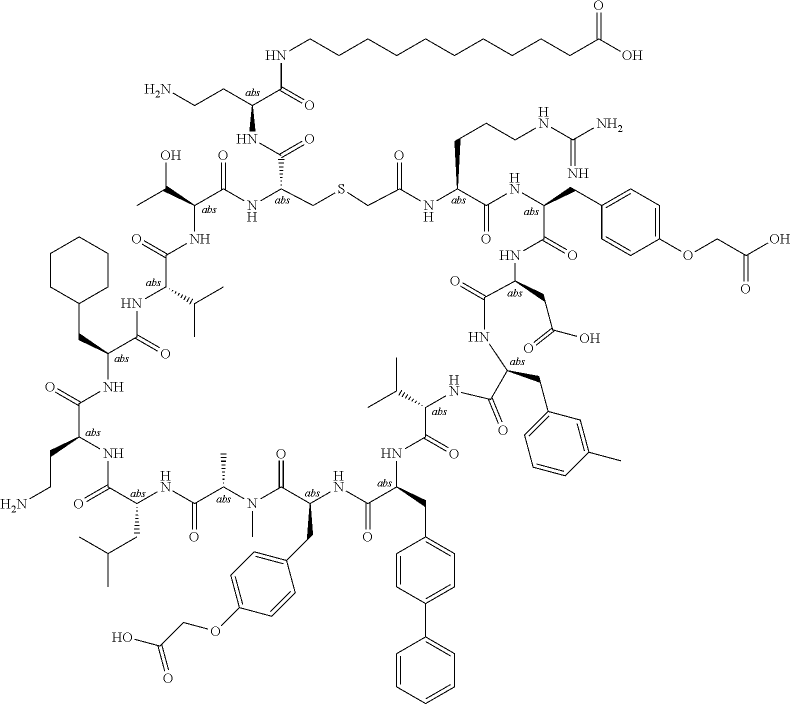 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 2308 was prepared on a 50 μmol scale. The yield of the product was 76.3 mg, and its estimated purity by LCMS analysis was 95.9%. Analysis condition B: Retention time=1.6 min; ESI-MS(+) m/z [M+3H]³⁺: 766.

### Preparation of Example 2309

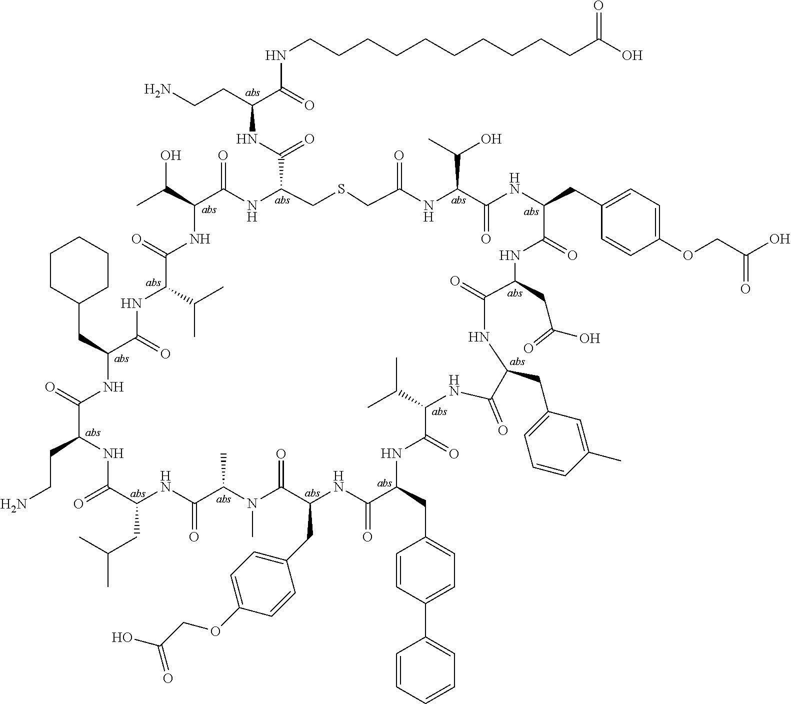 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 2309 was prepared on a 50 μmol scale. The yield of the product was 77.3 mg, and its estimated purity by LCMS analysis was 98.2%. Analysis condition A: Retention time=1.52 min; ESI-MS(+) m/z [M+2H]²⁺: 1120.1.

### Preparation of Example 2310

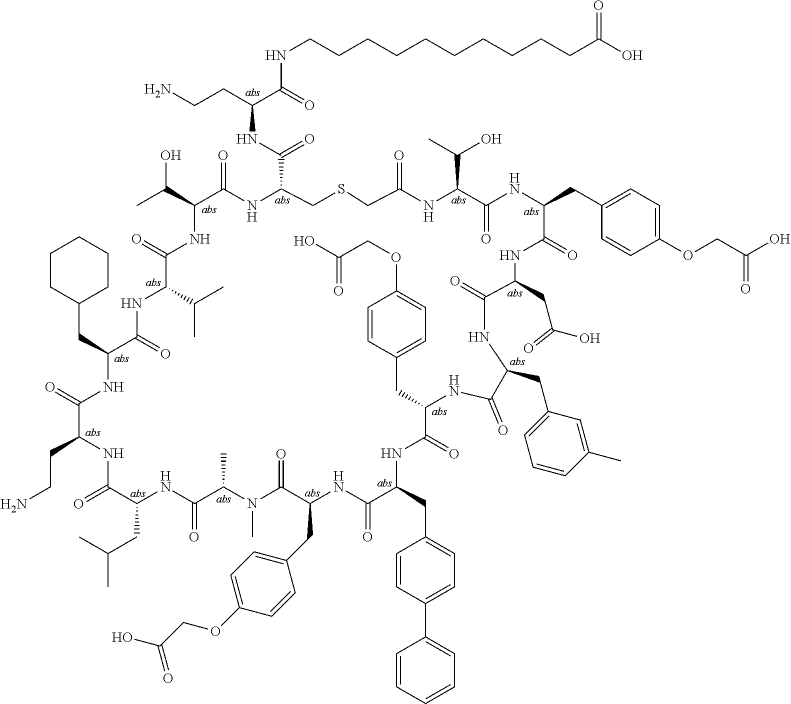 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2310 was prepared on a 50 μmol scale. The yield of the product was 99.9 mg, and its estimated purity by LCMS analysis was 97.7%. Analysis condition B: Retention time=1.73 min; ESI-MS(+) m/z [M+2H]²⁺: 1180.8.

### Preparation of Example 2311

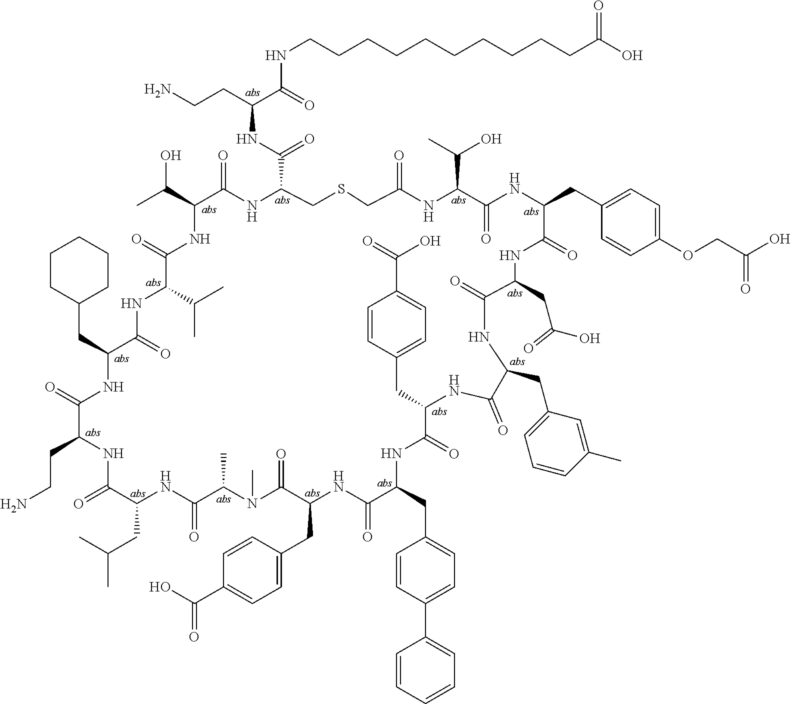 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2311 was prepared on a 50 μmol scale. The yield of the product was 5.6 mg, and its estimated purity by LCMS analysis was 95.2%. Analysis condition B: Retention time=1.92 min; ESI-MS(+) m/z [M+2H]²⁺: 1151.1.

### Preparation of Example 2312

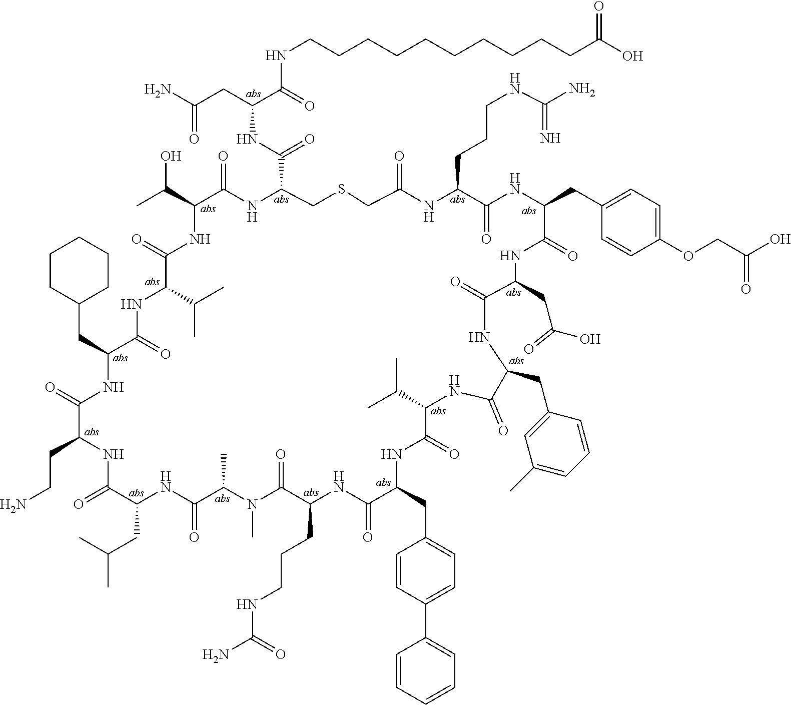 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2312 was prepared on a 50 μmol scale. The yield of the product was 24.6 mg, and its estimated purity by LCMS analysis was 96.6%. Analysis condition B: Retention time=1.87 min; ESI-MS(+) m/z [M+2H]²⁺: 1122.3.

### Preparation of Example 2313

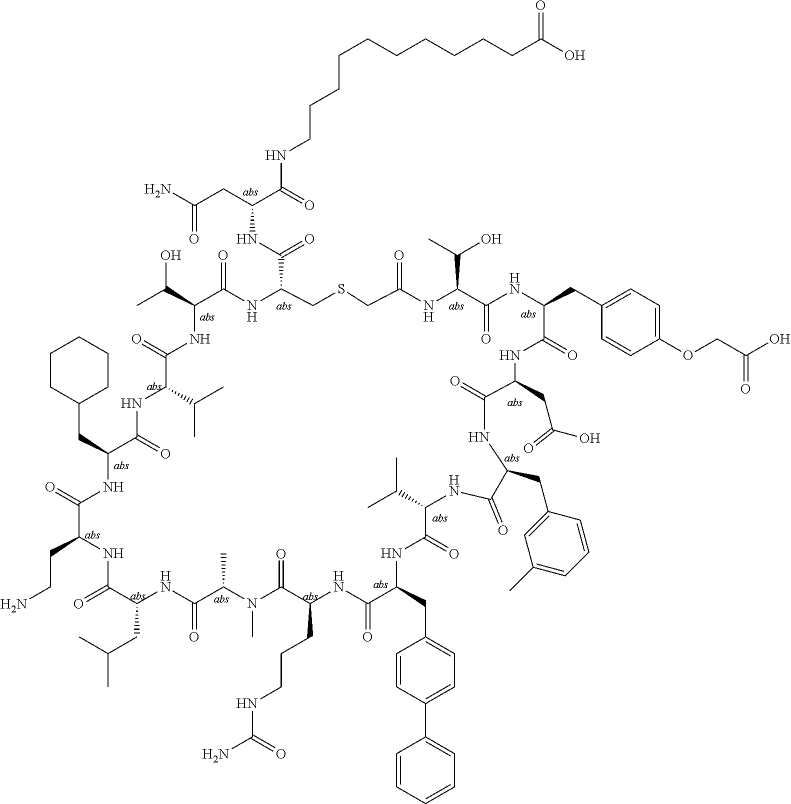 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2313 was prepared on a 50 μmol scale. The yield of the product was 74.5 mg, and its estimated purity by LCMS analysis was 97.2%. Analysis condition A: Retention time=1.77 min; ESI-MS(+) m/z [M+2H]²⁺: 1095.1.

### Preparation of Example 2314

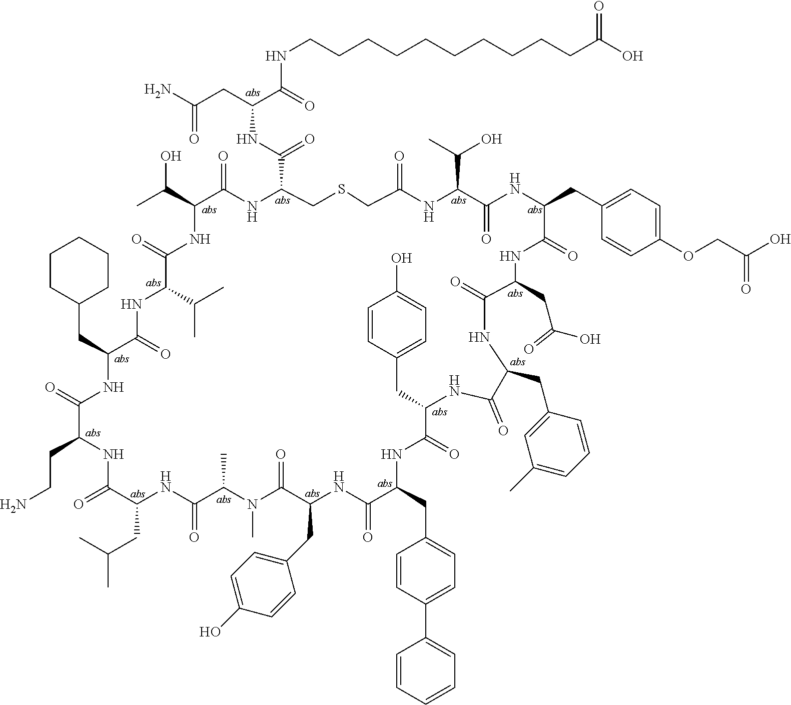 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 2314 was prepared on a 50 μmol scale. The yield of the product was 34.9 mg, and its estimated purity by LCMS analysis was 100%. Analysis condition A: Retention time=1.75 min; ESI-MS(+) m/z [M+2H]²⁺: 1130.1.

### Preparation of Example 2315

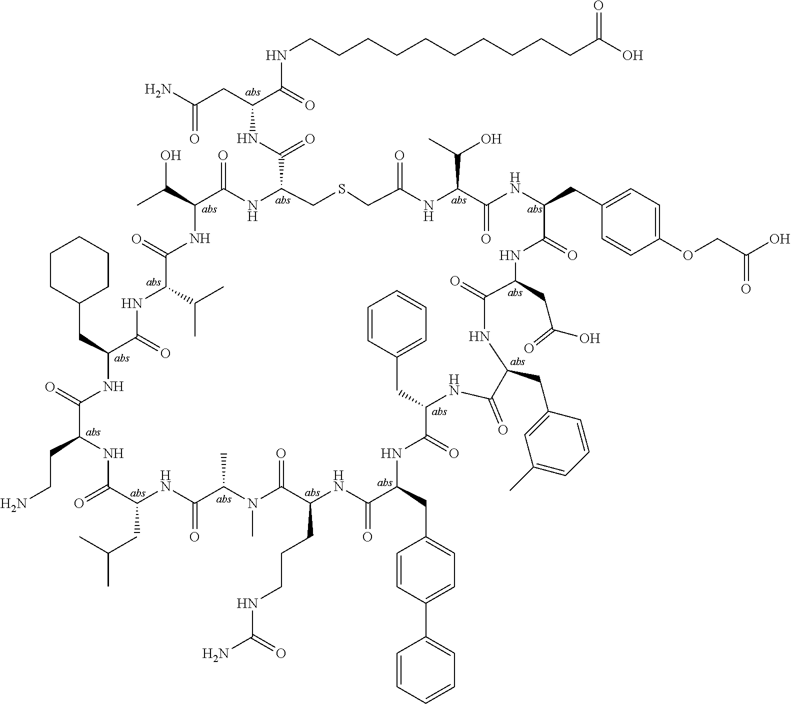 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccccc3)NC2=O)c1]

Example 2315 was prepared on a 50 μmol scale. The yield of the product was 35.1 mg, and its estimated purity by LCMS analysis was 96.4%. Analysis condition B: Retention time=2.05 min; ESI-MS(+) m/z [M+2H]²⁺: 1119.

### Preparation of Example 2316

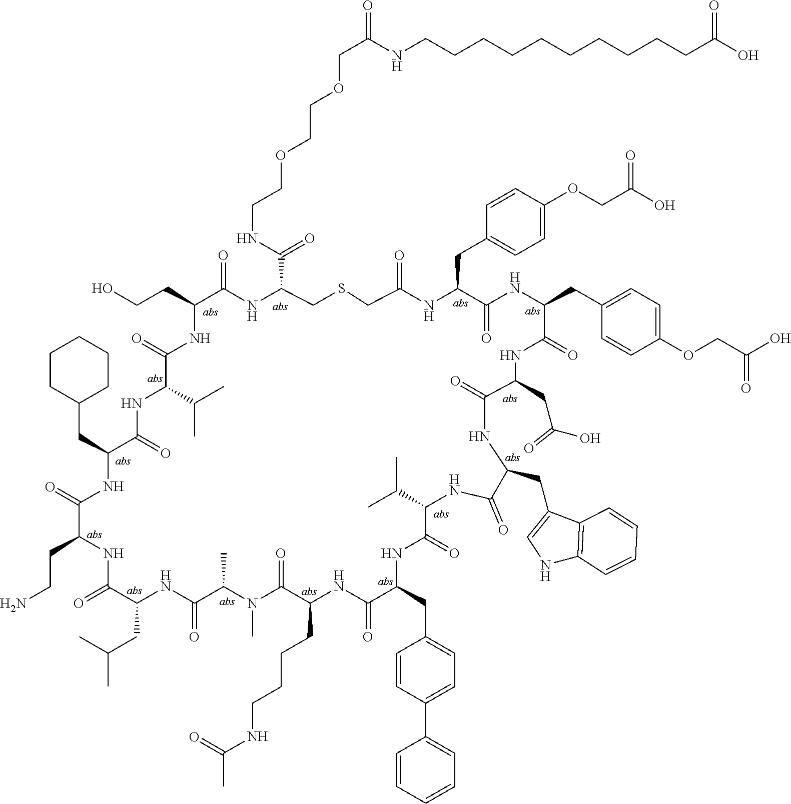 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2316 was prepared on a 50 μmol scale. The yield of the product was 64.8 mg, and its estimated purity by LCMS analysis was 98.1%. Analysis condition B: Retention time=2 min; ESI-MS(+) m/z [M+3H]³⁺: 793.4.

### Preparation of Example 2317

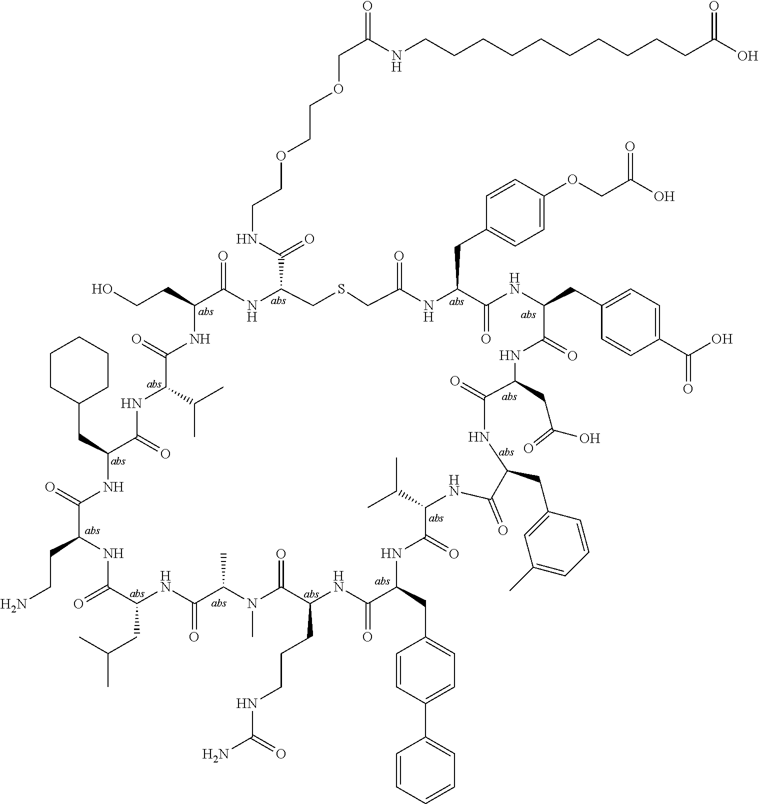 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2317 was prepared on a 50 μmol scale. The yield of the product was 11.1 mg, and its estimated purity by LCMS analysis was 98.1%. Analysis condition B: Retention time=2.04 min; ESI-MS(+) m/z [M+2H]²⁺: 1156.

### Preparation of Example 2318

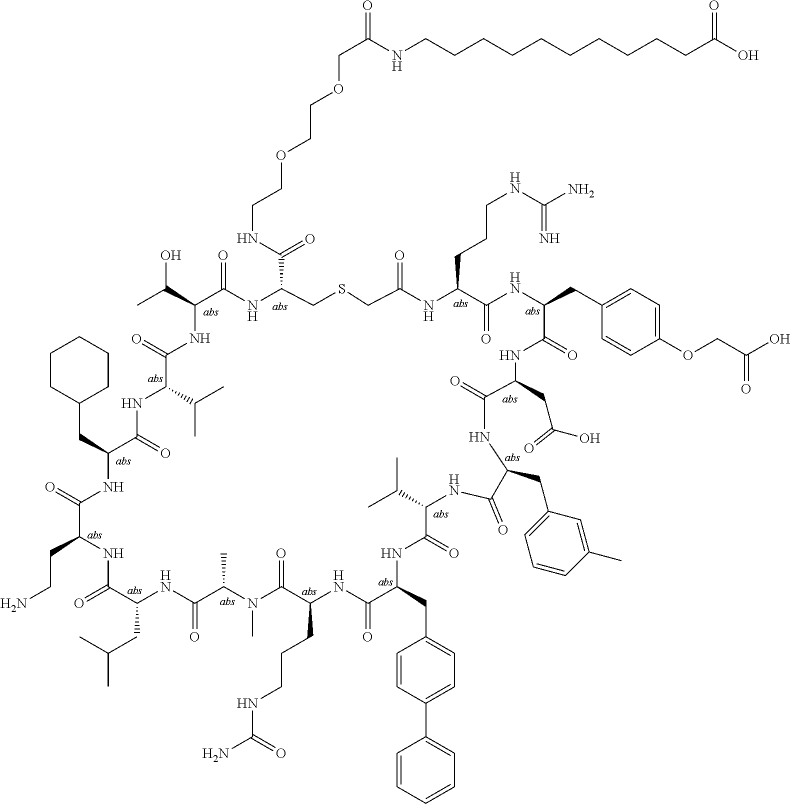 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2318 was prepared on a 50 μmol scale. The yield of the product was 74.6 mg, and its estimated purity by LCMS analysis was 96.9%. Analysis condition B: Retention time=1.92 min; ESI-MS(+) m/z [M+2H]²⁺: 1138.1.

### Preparation of Example 2319

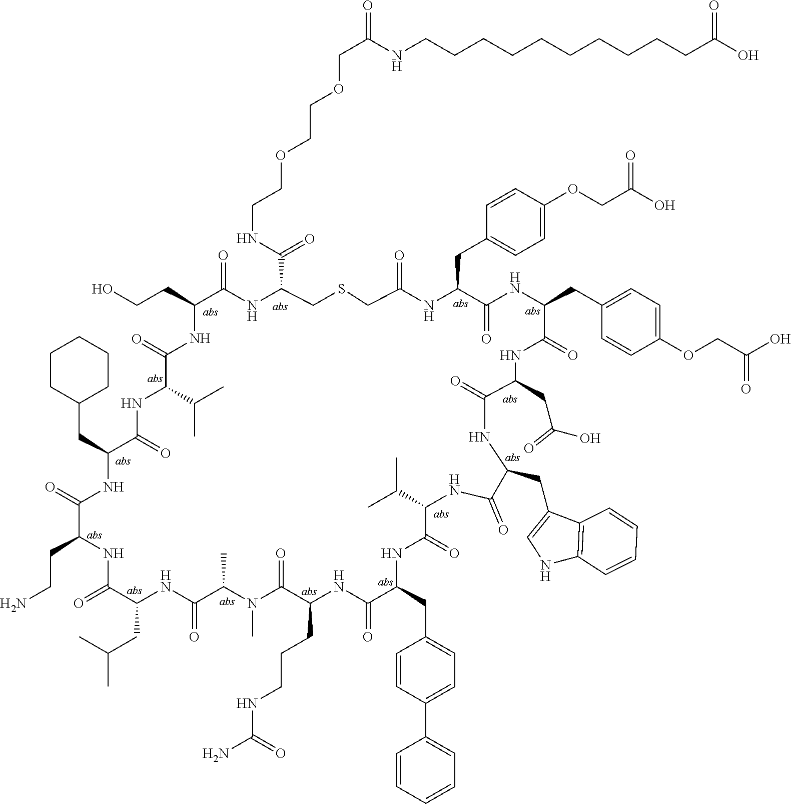 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Example 2319 was prepared on a 50 μmol scale. The yield of the product was 62.3 mg, and its estimated purity by LCMS analysis was 92.5%. Analysis condition A: Retention time=1.59 min; ESI-MS(+) m/z [M+2H]²⁺: 1182.9.

### Preparation of Example 2320

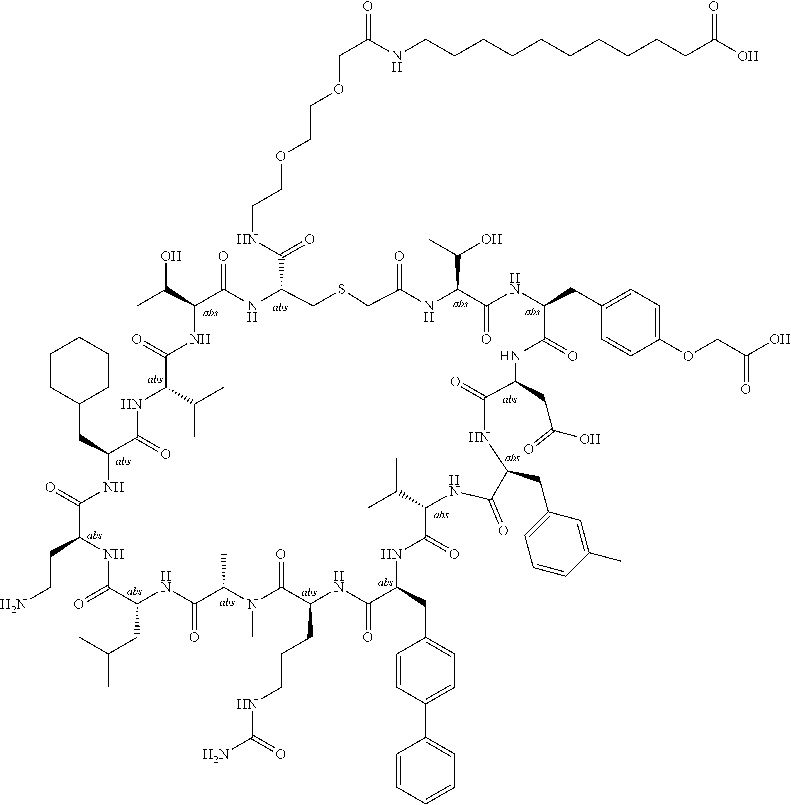 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2320 was prepared on a 50 μmol scale. The yield of the product was 48.1 mg, and its estimated purity by LCMS analysis was 99.4%. Analysis condition B: Retention time=1.85 min; ESI-MS(+) m/z [M+2H]²⁺: 1110.3.

### Preparation of Example 2321

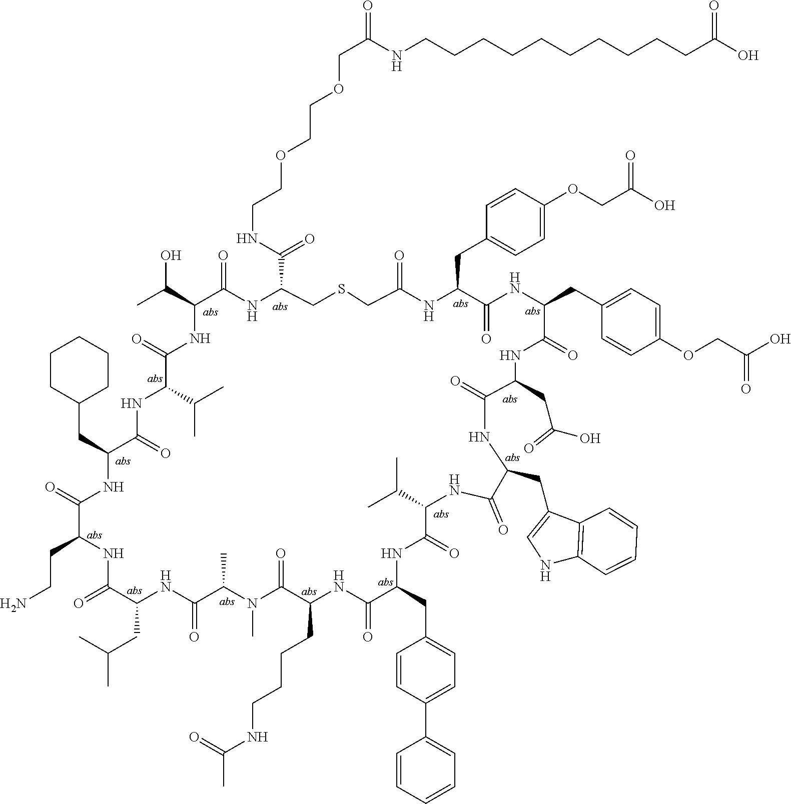 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2321 was prepared on a 50 μmol scale. The yield of the product was 35.6 mg, and its estimated purity by LCMS analysis was 100%. Analysis condition A: Retention time=1.71 min; ESI-MS(+) m/z [M+2H]²⁺: 1189.4.

### Preparation of Example 2322

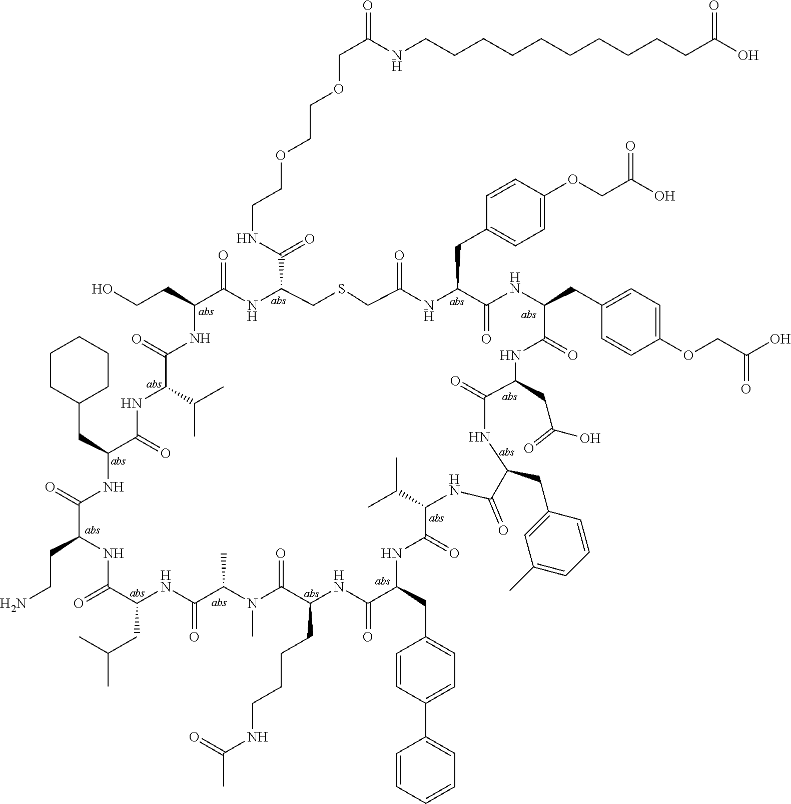 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2322 was prepared on a 50 μmol scale. The yield of the product was 67.2 mg, and its estimated purity by LCMS analysis was 95%. Analysis condition B: Retention time=2.06 min; ESI-MS(+) m/z [M+2H]²⁺: 1162.1.

### Preparation of Example 2323

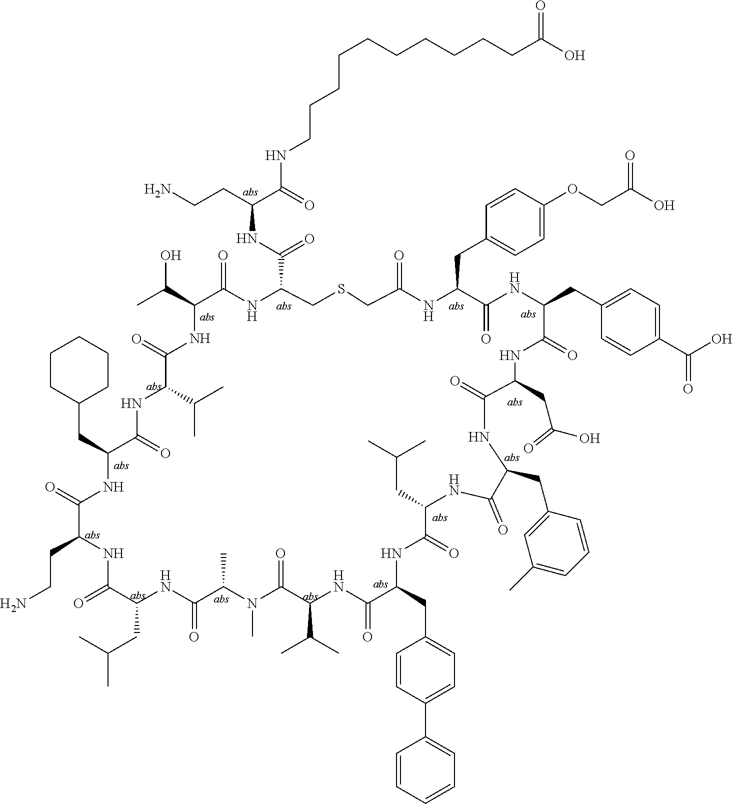 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2323 was prepared on a 50 μmol scale. The yield of the product was 56.5 mg, and its estimated purity by LCMS analysis was 98.9%. Analysis condition A: Retention time=1.68 min; ESI-MS(+) m/z [M+2H]²⁺: 1111.

### Preparation of Example 2324

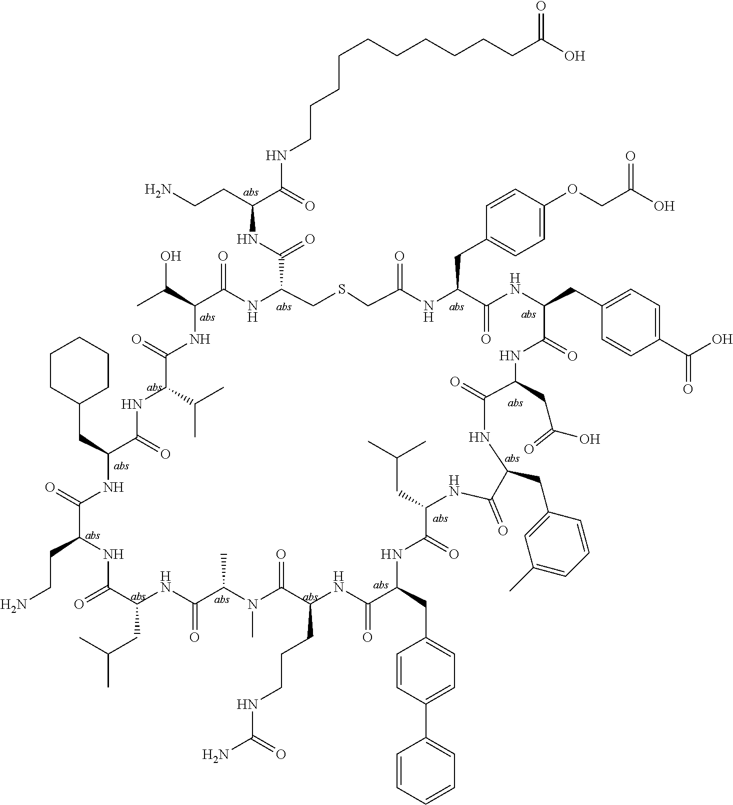 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](CC(C)C)NC2=O)c1]

Example 2324 was prepared on a 50 μmol scale. The yield of the product was 44.5 mg, and its estimated purity by LCMS analysis was 97.6%. Analysis condition B: Retention time=1.94 min; ESI-MS(+) m/z [M+2H]²⁺: 1140.1.

### Preparation of Example 2325

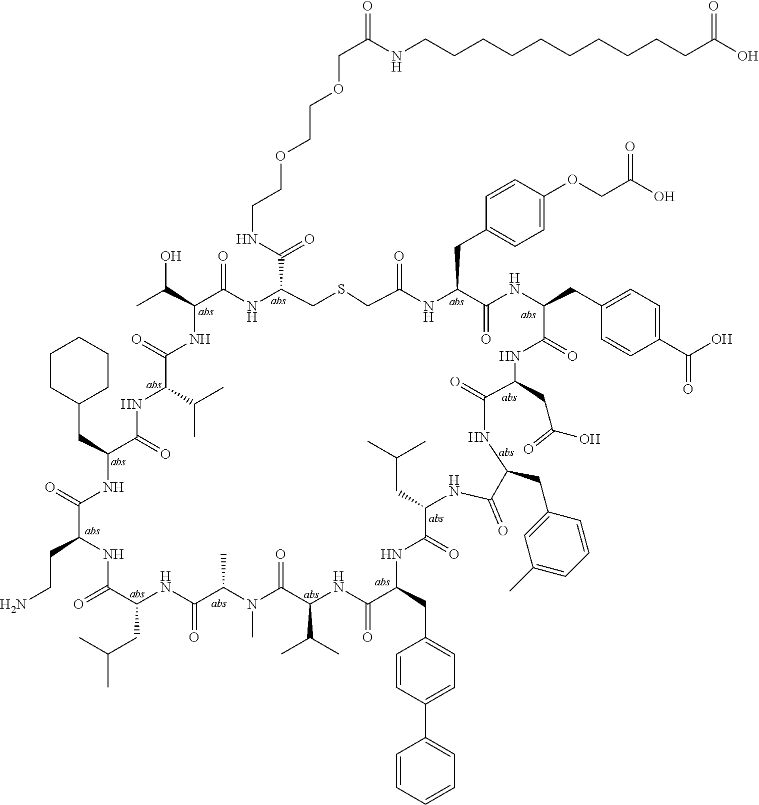 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2325 was prepared on a 50 μmol scale. The yield of the product was 36.1 mg, and its estimated purity by LCMS analysis was 97.9%. Analysis condition A: Retention time=1.77 min; ESI-MS(+) m/z [M+2H]²⁺: 1134.1.

### Preparation of Example 2326

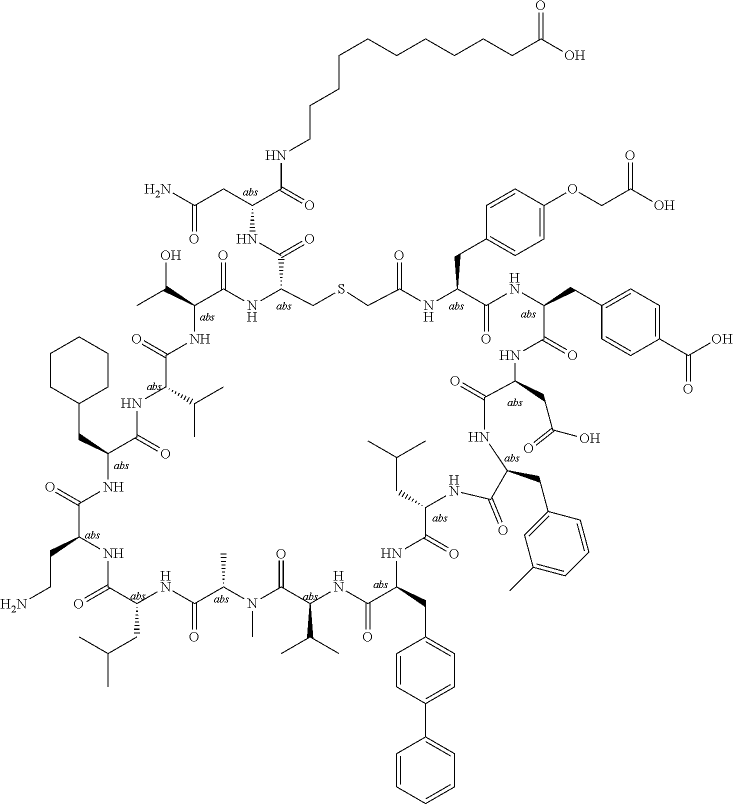 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2326 was prepared on a 50 μmol scale. The yield of the product was 1.9 mg, and its estimated purity by LCMS analysis was 98.4%. Analysis condition A: Retention time=1.75 min; ESI-MS(+) m/z [M+2H]²⁺: 1118.

### Preparation of Example 2327

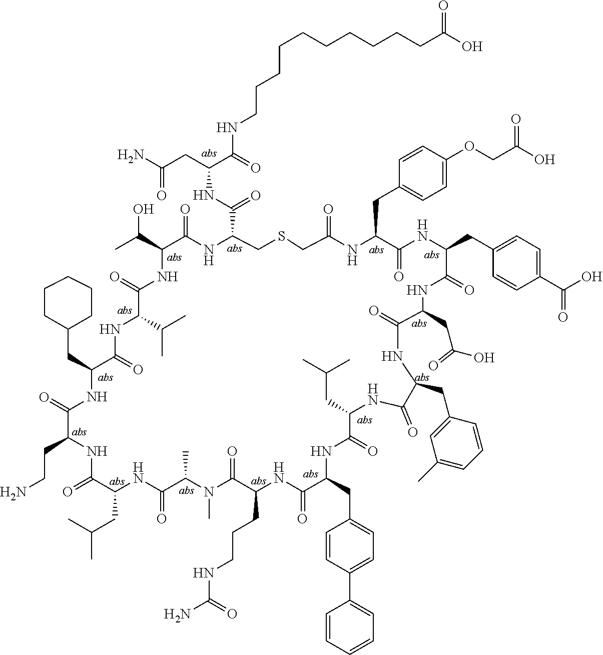 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](CC(C)C)NC2=O)c1]

Example 2327 was prepared on a 50 μmol scale. The yield of the product was 34.9 mg, and its estimated purity by LCMS analysis was 97.2%. Analysis condition A: Retention time=1.71 min; ESI-MS(+) m/z [M+2H]²⁺: 1146.8.

### Preparation of Example 2328

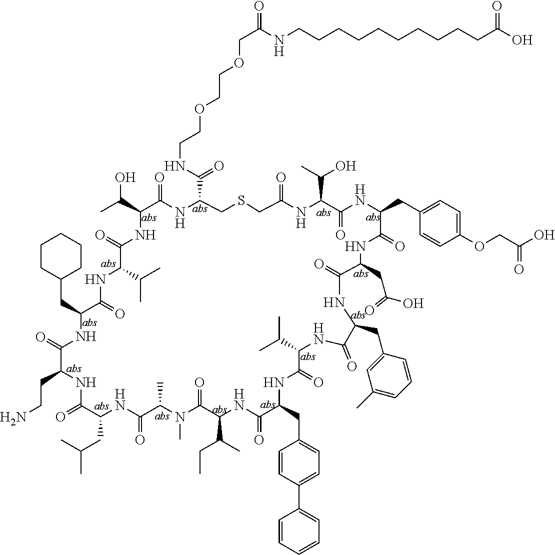 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2328 was prepared on a 50 μmol scale. The yield of the product was 58.4 mg, and its estimated purity by LCMS analysis was 98.7%. Analysis condition B: Retention time=2 min; ESI-MS(+) m/z [M+2H]²⁺: 1089.1.

### Preparation of Example 2329

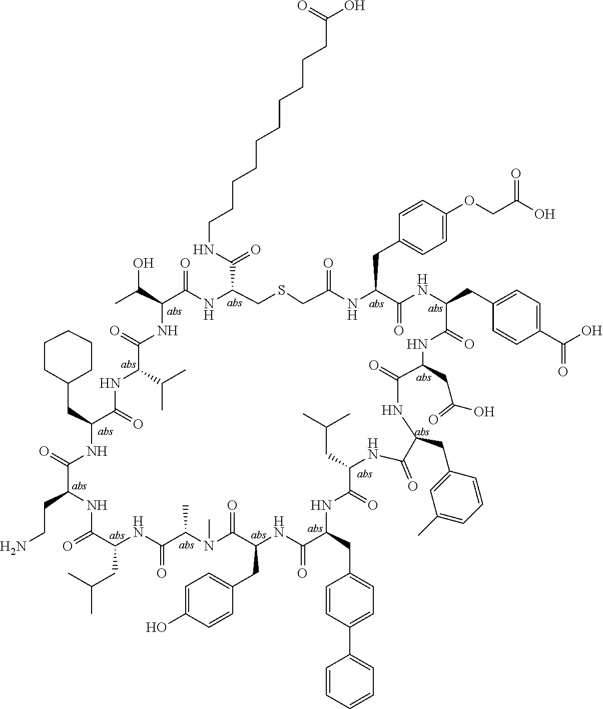 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CC(C)C)NC2=O)c1]

Example 2329 was prepared on a 50 μmol scale. The yield of the product was 41.3 mg, and its estimated purity by LCMS analysis was 95.3%. Analysis condition A: Retention time=1.81 min; ESI-MS(+) m/z [M+2H]²⁺: 1093.2.

### Preparation of Example 2330

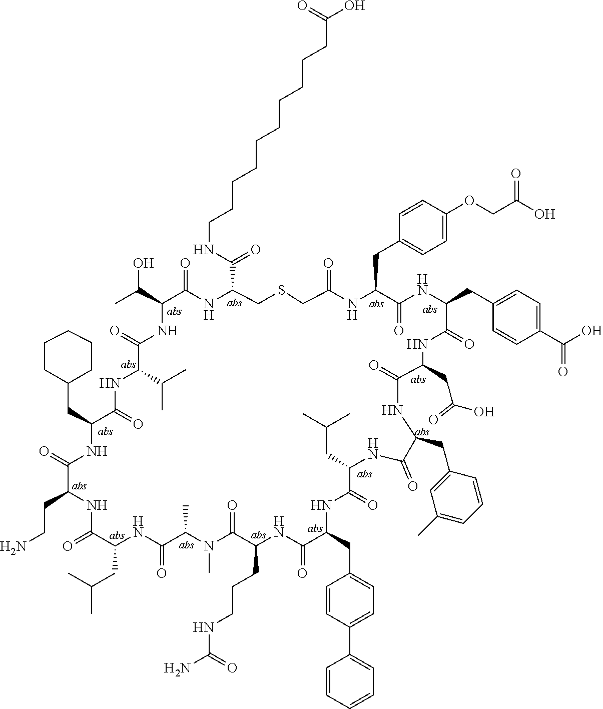 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](CC(C)C)NC2=O)c1]

Example 2330 was prepared on a 50 μmol scale. The yield of the product was 67.5 mg, and its estimated purity by LCMS analysis was 98.3%. Analysis condition A: Retention time=1.83 min; ESI-MS(+) m/z [M+2H]²⁺: 1090.2.

### Preparation of Example 2331

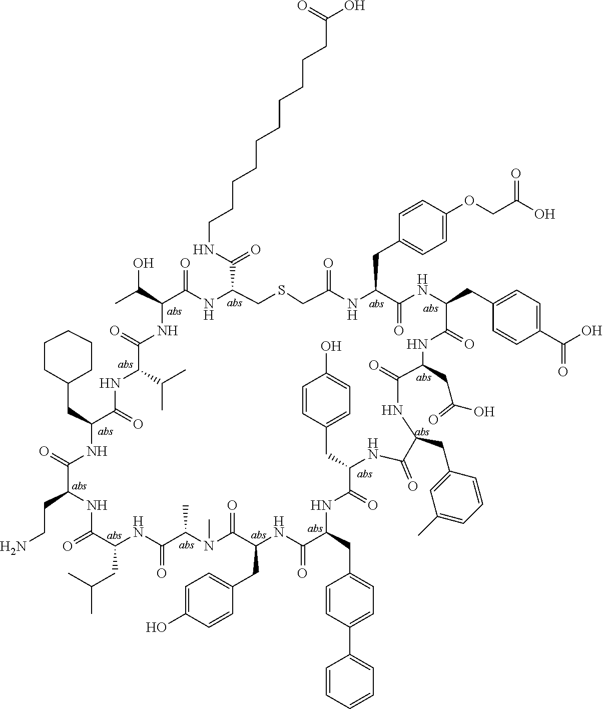 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Example 2331 was prepared on a 50 μmol scale. The yield of the product was 18.1 mg, and its estimated purity by LCMS analysis was 98.3%. Analysis condition B: Retention time=2 min; ESI-MS(+) m/z [M+2H]²⁺: 1118.

### Preparation of Example 2332

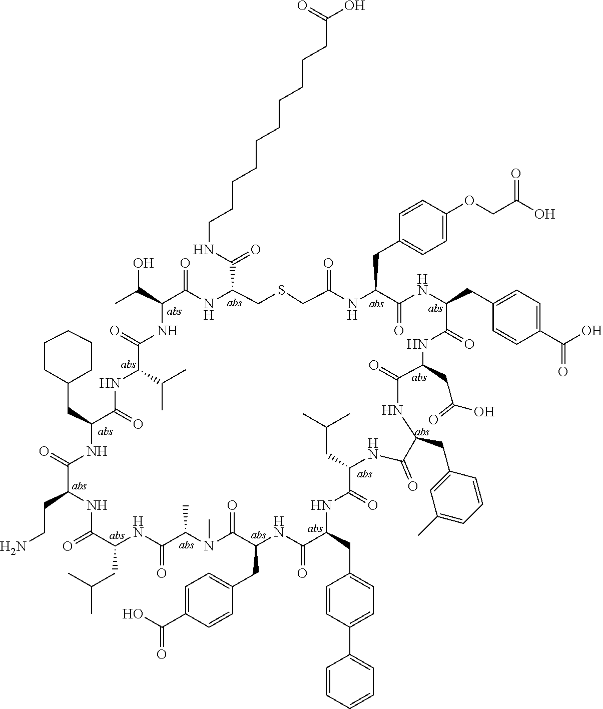 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](CC(C)C)NC2=O)c1]

Example 2332 was prepared on a 50 μmol scale. The yield of the product was 30.7 mg, and its estimated purity by LCMS analysis was 95.4%. Analysis condition B: Retention time=2.07 min; ESI-MS(+) m/z [M+2H]²⁺: 1106.9.

### Preparation of Example 2333

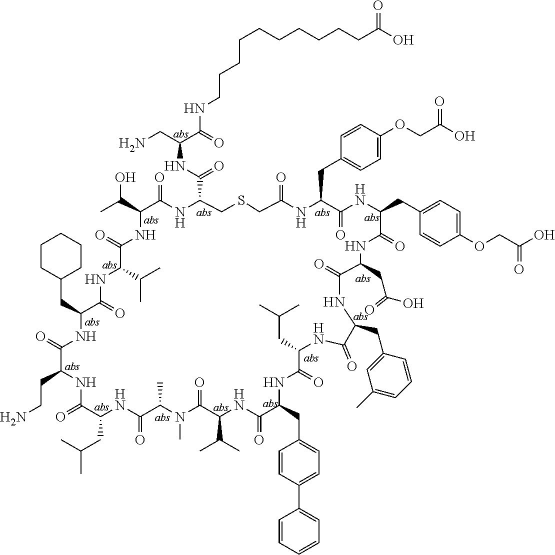 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2333 was prepared on a 50 μmol scale. The yield of the product was 72.8 mg, and its estimated purity by LCMS analysis was 96.5%. Analysis condition B: Retention time=1.92 min; ESI-MS(+) m/z [M+2H]²⁺: 1118.9.

### Preparation of Example 2334

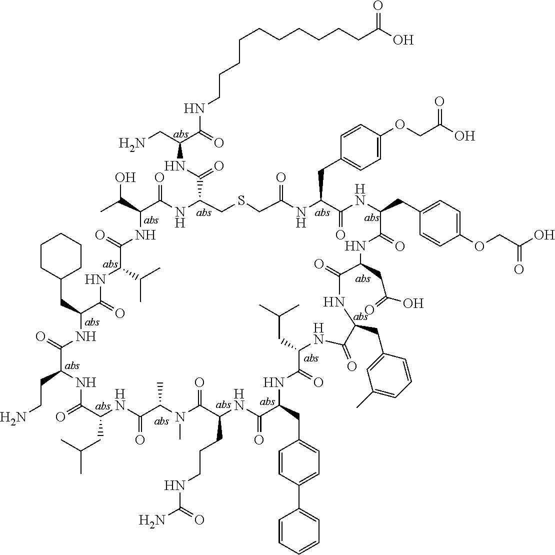 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](CC(C)C)NC2=O)c1]

Example 2334 was prepared on a 50 μmol scale. The yield of the product was 55.7 mg, and its estimated purity by LCMS analysis was 95.3%. Analysis condition A: Retention time=1.69 min; ESI-MS(+) m/z [M+2H]²⁺: 1148.1.

### Preparation of Example 2335

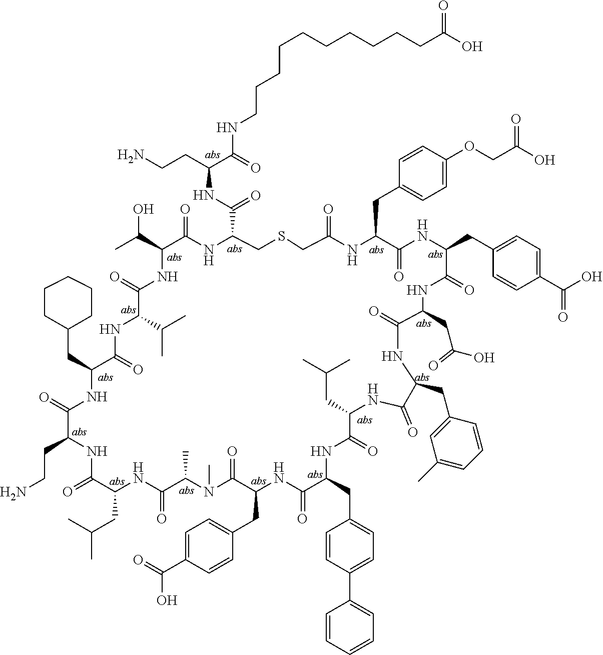 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](CC(C)C)NC2=O)c1]

Example 2335 was prepared on a 50 μmol scale. The yield of the product was 44.7 mg, and its estimated purity by LCMS analysis was 95.3%. Analysis condition A: Retention time=1.56 min; ESI-MS(+) m/z [M+2H]²⁺: 1157.1.

### Preparation of Example 2336

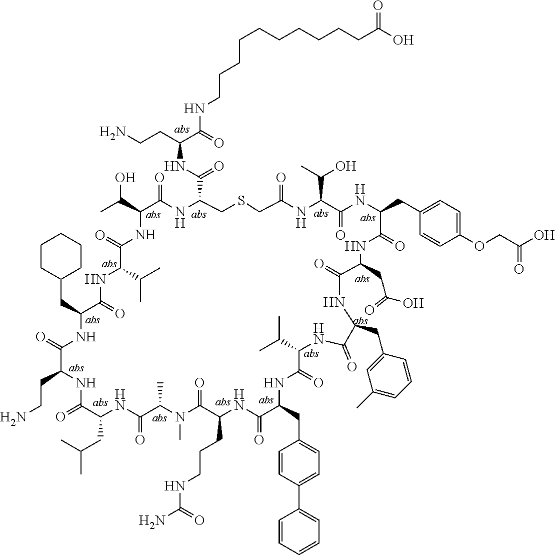 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2336 was prepared on a 50 μmol scale. The yield of the product was 27.8 mg, and its estimated purity by LCMS analysis was 98%. Analysis condition B: Retention time=1.65 min; ESI-MS(+) m/z [M+2H]²⁺: 1088.

### Preparation of Example 2337

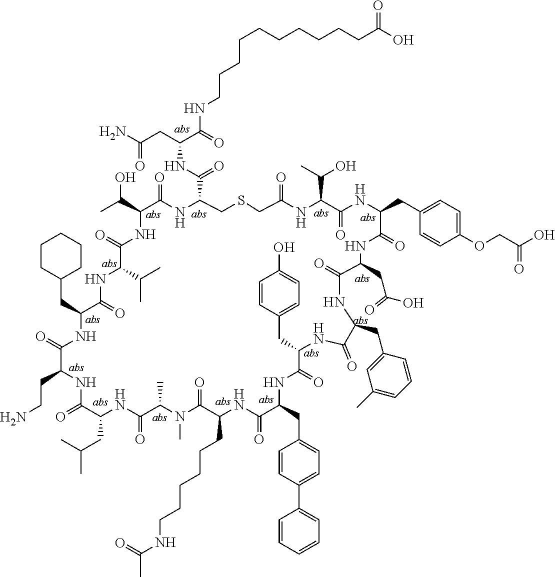 [SMILES: CC(=O)NCCCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2337 was prepared on a 50 μmol scale. The yield of the product was 42.8 mg, and its estimated purity by LCMS analysis was 100%. Analysis condition A: Retention time=1.68 min; ESI-MS(+) m/z [M+2H]²⁺: 1134.1.

### Preparation of Example 2338

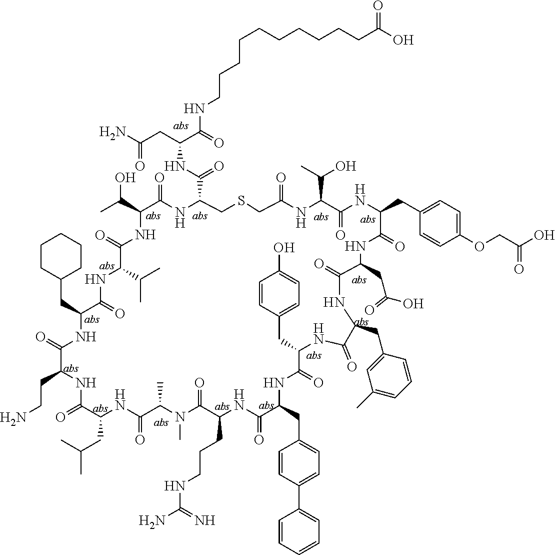 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](Cc3ccc(O)cc3)NC2=O)c1]

Example 2338 was prepared on a 50 μmol scale. The yield of the product was 55.1 mg, and its estimated purity by LCMS analysis was 95.7%. Analysis condition A: Retention time=1.67 min; ESI-MS(+) m/z [M+2H]²⁺: 1127.

### Preparation of Example 2339

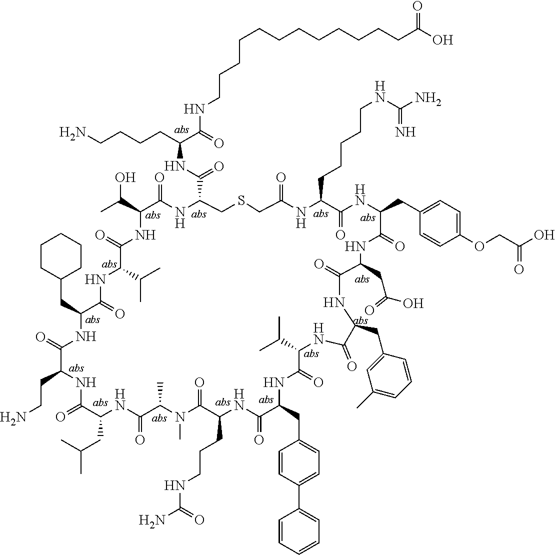 [SMILES: Cc1cccc(C[C@@H]2NC(=O)[C@H](CC(=O)O)NC(=O)[C@H](Cc3ccc(OCC(=O)O)cc3)NC(=O)[C@H](CCCCCNC(=N)N)NC(=O)CSC[C@@H](C(=O)N[C@@H](CCCCN)C(=O)NCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2339 was prepared on a 50 μmol scale. The yield of the product was 48.4 mg, and its estimated purity by LCMS analysis was 95.1%. Analysis condition A: Retention time=1.63 min; ESI-MS(+) m/z [M+2H]²⁺: 1129.3.

### Preparation of Example 2340

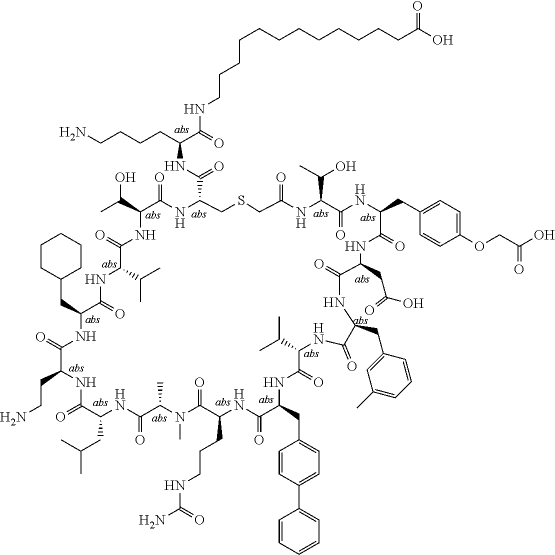 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCCN)C(=O)NCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2340 was prepared on a 50 μmol scale. The yield of the product was 9.8 mg, and its estimated purity by LCMS analysis was 97.2%. Analysis condition A: Retention time=1.65 min; ESI-MS(+) m/z [M+2H]²⁺: 1102.3.

### Preparation of Example 2341

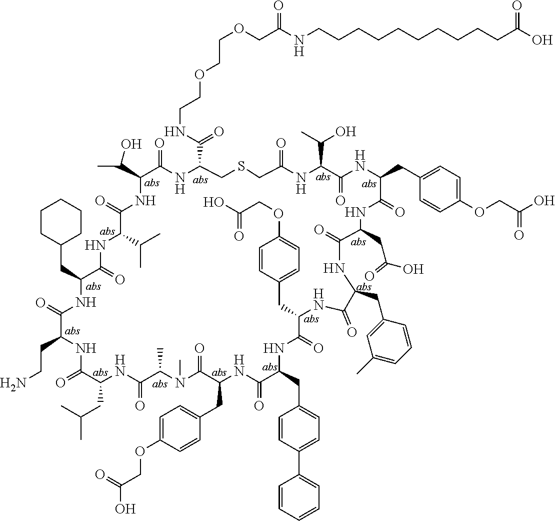 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2341 was prepared on a 50 μmol scale. The yield of the product was 86.7 mg, and its estimated purity by LCMS analysis was 97.4%. Analysis condition B: Retention time=2.06 min; ESI-MS(+) m/z [M+2H]²⁺: 1204.1.

### Preparation of Example 2342

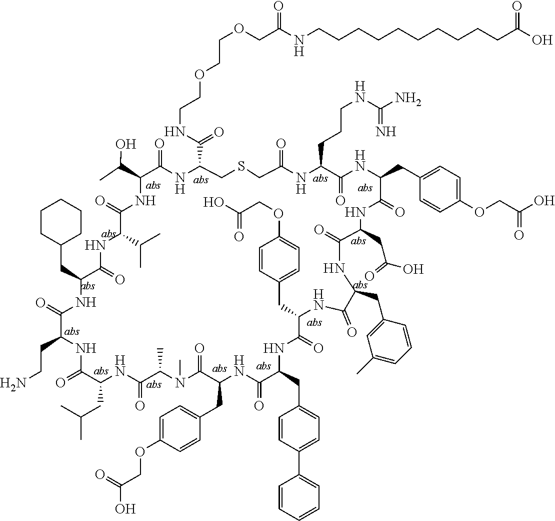 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2342 was prepared on a 50 μmol scale. The yield of the product was 63.1 mg, and its estimated purity by LCMS analysis was 95.8%. Analysis condition B: Retention time=1.88 min; ESI-MS(+) m/z [M+2H]²⁺: 1231.

### Preparation of Example 2343

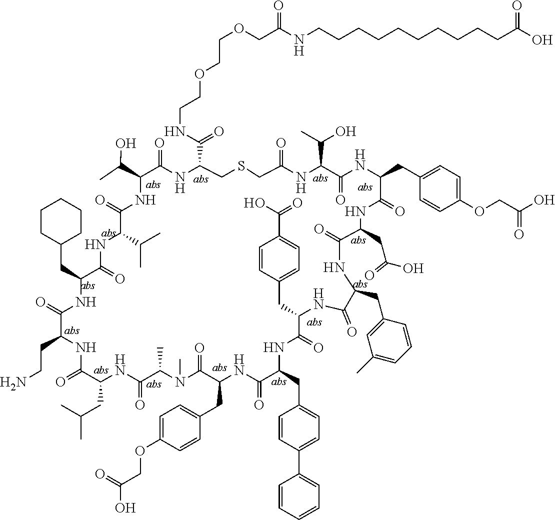 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2343 was prepared on a 50 μmol scale. The yield of the product was 102.9 mg, and its estimated purity by LCMS analysis was 97.3%. Analysis condition A: Retention time=1.53 min; ESI-MS(+) m/z [M+2H]²⁺: 1187.8.

### Preparation of Example 2344

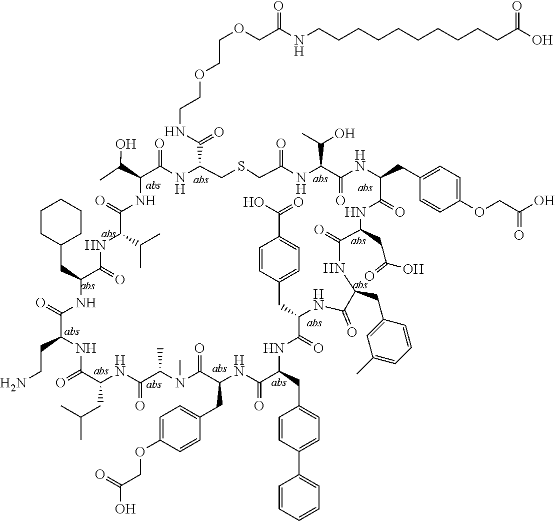 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2344 was prepared on a 50 μmol scale. The yield of the product was 56.2 mg, and its estimated purity by LCMS analysis was 98.1%. Analysis condition A: Retention time=1.56 min; ESI-MS(+) m/z [M+2H]²⁺: 1174.

### Preparation of Example 2345

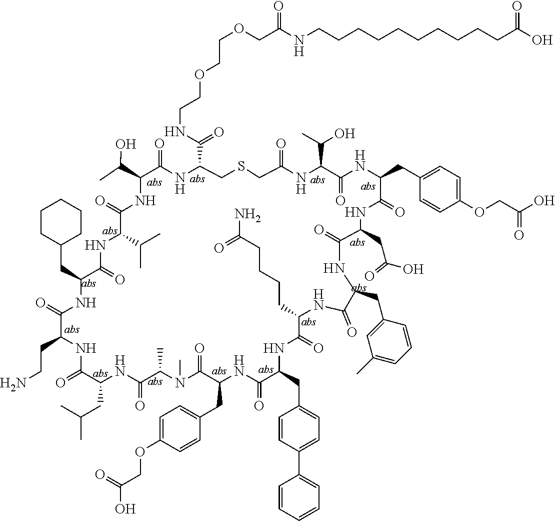 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCCC(N)=O)NC2=O)c1]

Example 2345 was prepared on a 50 μmol scale. The yield of the product was 23.6 mg, and its estimated purity by LCMS analysis was 96.8%. Analysis condition B: Retention time=1.91 min; ESI-MS(+) m/z [M+2H]²⁺: 1172.1.

### Preparation of Example 2346

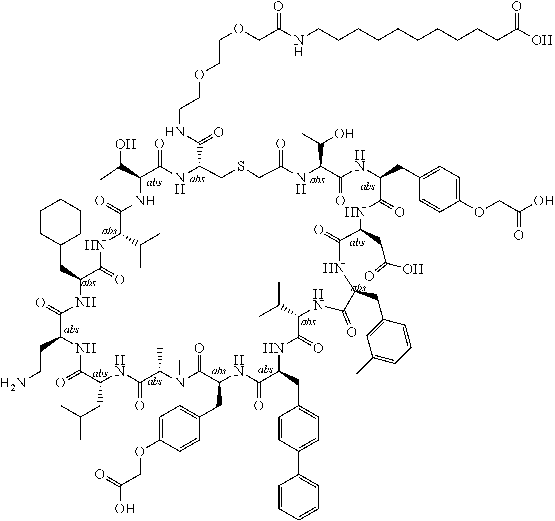 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Example 2346 was prepared on a 50 μmol scale. The yield of the product was 32.3 mg, and its estimated purity by LCMS analysis was 96.6%. Analysis condition B: Retention time=1.97 min; ESI-MS(+) m/z [M+2H]²⁺: 1142.

### Preparation of Example 2347

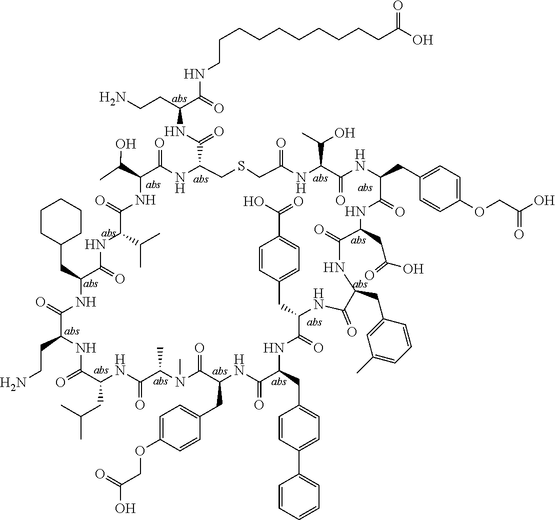 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2347 was prepared on a 50 μmol scale. The yield of the product was 64.5 mg, and its estimated purity by LCMS analysis was 98.7%. Analysis condition A: Retention time=1.52 min; ESI-MS(+) m/z [M+2H]²⁺: 1166.1.

### Preparation of Example 2348

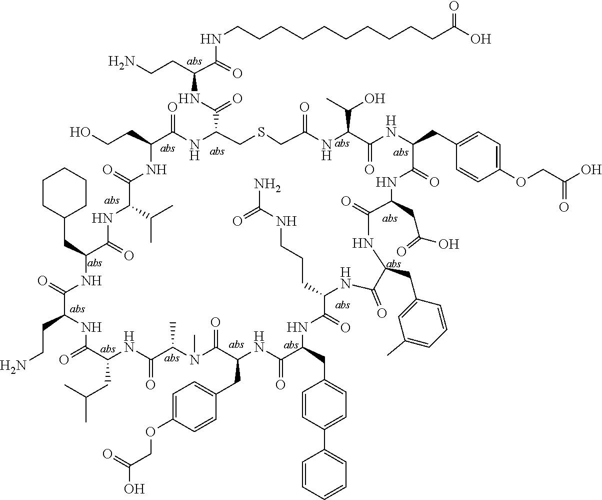 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCNC(N)=O)NC2=O)c1]

Example 2348 was prepared on a 50 μmol scale. The yield of the product was 8.7 mg, and its estimated purity by LCMS analysis was 98.3%. Analysis condition A: Retention time=1.34 min; ESI-MS(+) m/z [M+2H]²⁺: 1149.1.

### Preparation of Example 2349

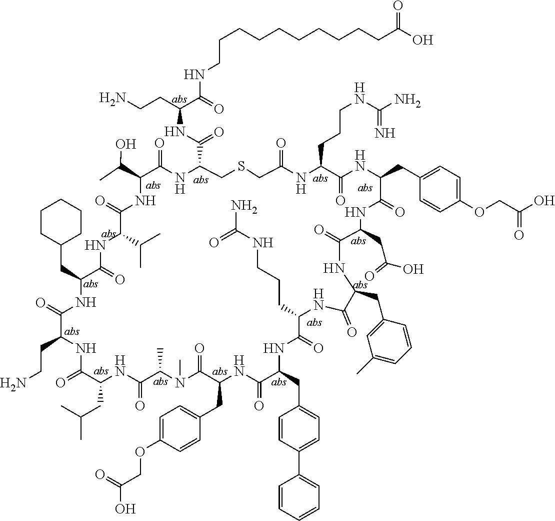 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCNC(N)=O)NC2=O)c1]

Example 2349 was prepared on a 50 μmol scale. The yield of the product was 1.3 mg, and its estimated purity by LCMS analysis was 95.8%. Analysis condition B: Retention time=1.52 min; ESI-MS(+) m/z [M+3H]³⁺: 784.5.

### Preparation of Example 2350

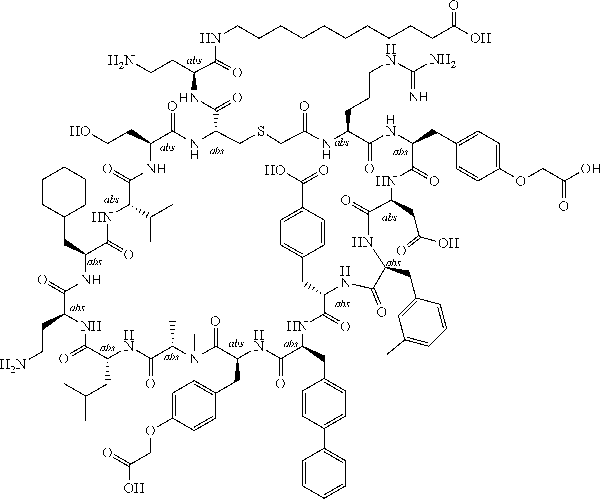 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2350 was prepared on a 50 μmol scale. The yield of the product was 4.1 mg, and its estimated purity by LCMS analysis was 99.2%. Analysis condition A: Retention time=1.5 min; ESI-MS(+) m/z [M+3H]³⁺: 796.4.

### Preparation of Example 2351

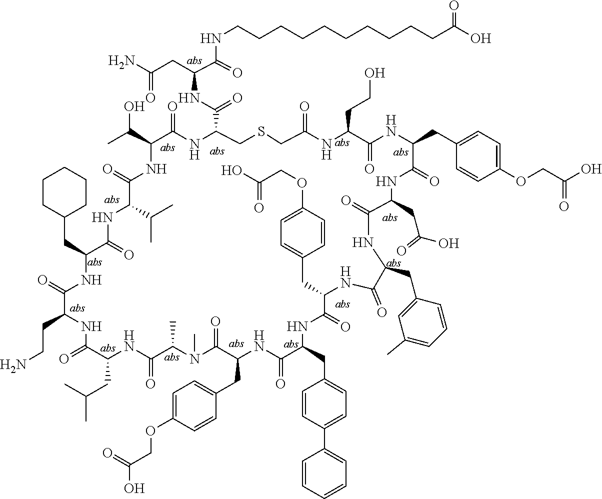 [SMILES: Cc1cccc(C[C@@H]2NC(=O)[C@H](CC(=O)O)NC(=O)[C@H](Cc3ccc(OCC(=O)O)cc3)NC(=O)[C@H](CCO)NC(=O)CSC[C@@H](C(=O)N[C@@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2351 was prepared on a 50 μmol scale. The yield of the product was 18.2 mg, and its estimated purity by LCMS analysis was 97.5%. Analysis condition B: Retention time=1.83 min; ESI-MS(+) m/z [M+2H]²⁺: 1180.9.

### Preparation of Example 2352

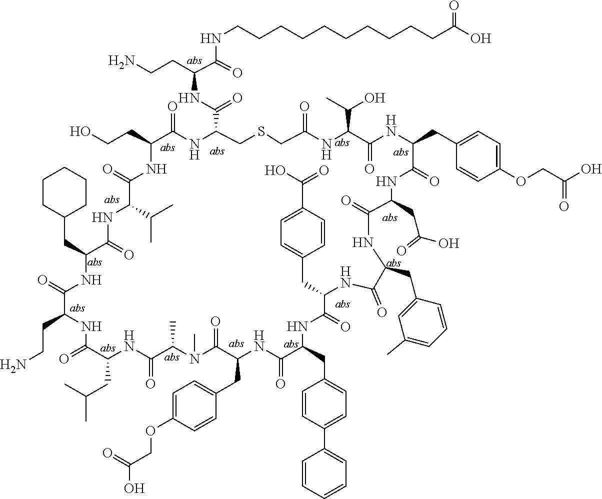 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2352 was prepared on a 50 μmol scale. The yield of the product was 8 mg, and its estimated purity by LCMS analysis was 97.3%. Analysis condition B: Retention time=1.81 min; ESI-MS(+) m/z [M+2H]²⁺: 1166.1.

### Preparation of Example 2353

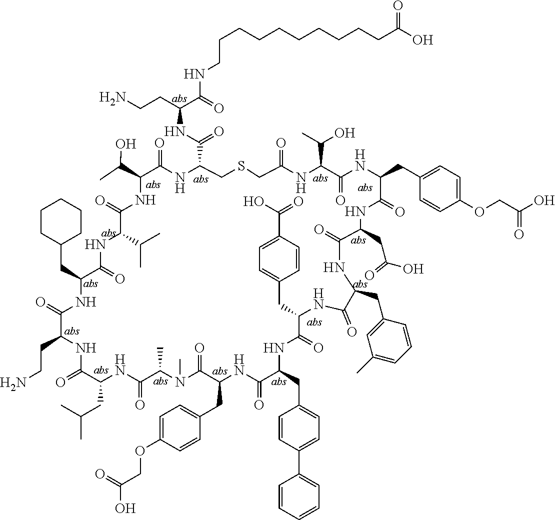 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2353 was prepared on a 50 μmol scale. The yield of the product was 21.1 mg, and its estimated purity by LCMS analysis was 95.6%. Analysis condition B: Retention time=1.84 min; ESI-MS(+) m/z [M+2H]²⁺: 1165.9.

### Preparation of Example 2354

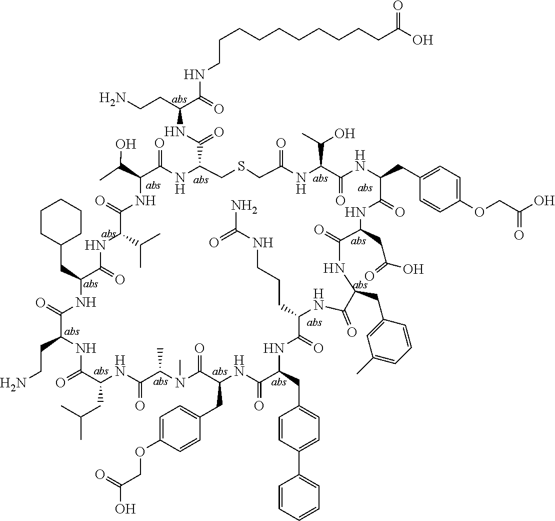 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](CCCNC(N)=O)NC2=O)c1]

Example 2354 was prepared on a 50 μmol scale. The yield of the product was 24.1 mg, and its estimated purity by LCMS analysis was 98.3%. Analysis condition B: Retention time=1.78 min; ESI-MS(+) m/z [M+2H]²⁺: 1149.2.

### Preparation of Example 2355

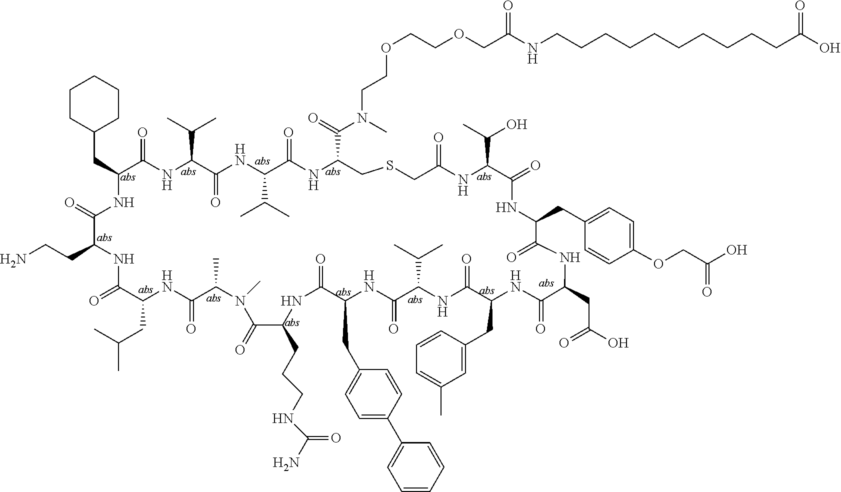 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](C(C)C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2355 was prepared on a 50 μmol scale. The yield of the product was 10.3 mg, and its estimated purity by LCMS analysis was 95.1%. Analysis condition B: Retention time=1.97 min; ESI-MS(+) m/z [M+2H]²⁺: 1117.2.

### Preparation of Example 2356

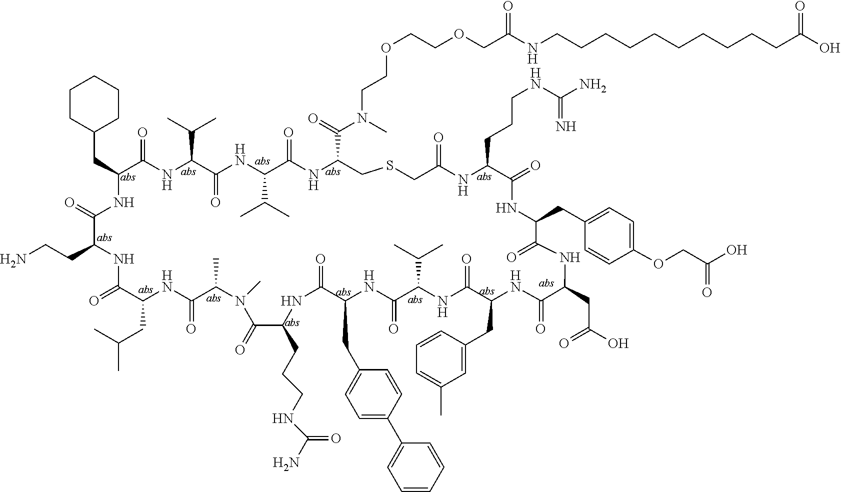 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)CCOCCOCC(=O)NCCCCCCCCCCC(=O)O)NC(=O)[C@H](C(C)C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2356 was prepared on a 50 μmol scale. The yield of the product was 29.6 mg, and its estimated purity by LCMS analysis was 96.9%. Analysis condition B: Retention time=1.85 min; ESI-MS(+) m/z [M+2H]²⁺: 1145.1.

### Preparation of Example 2357

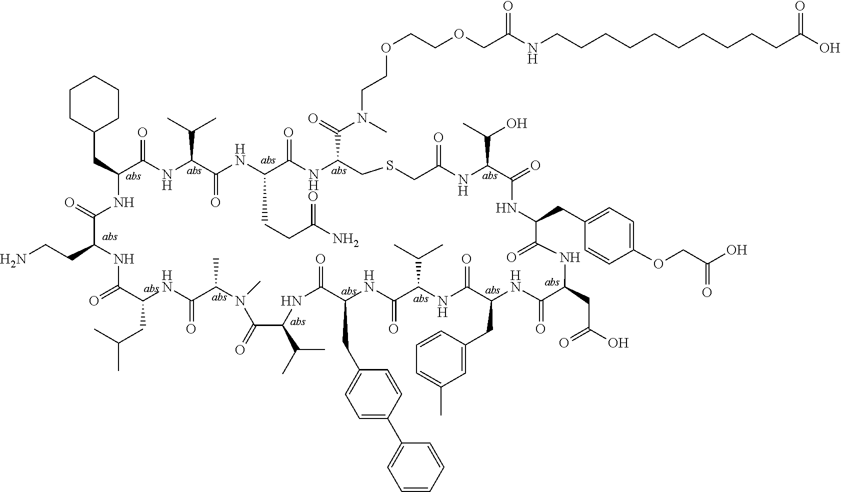 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2357 was prepared on a 50 μmol scale. The yield of the product was 11.2 mg, and its estimated purity by LCMS analysis was 99.3%. Analysis condition B: Retention time=1.97 min; ESI-MS(+) m/z [M+2H]²⁺: 1102.2.

### Preparation of Example 2358

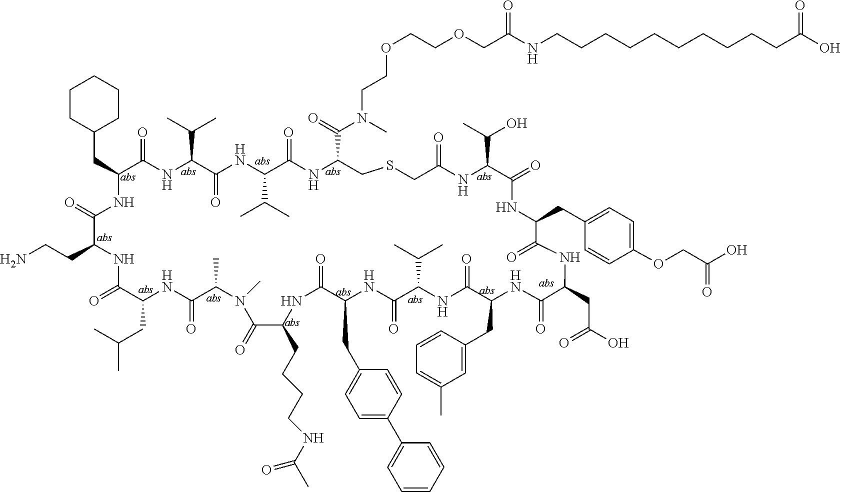 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)CCOCCOCC(=O)NCCCCCCCCCCC(=O)O)NC(=O)[C@H](C(C)C)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2358 was prepared on a 50 μmol scale. The yield of the product was 16.7 mg, and its estimated purity by LCMS analysis was 96.4%. Analysis condition B: Retention time=1.97 min; ESI-MS(+) m/z [M+2H]²⁺: 1124.2.

### Preparation of Example 2359

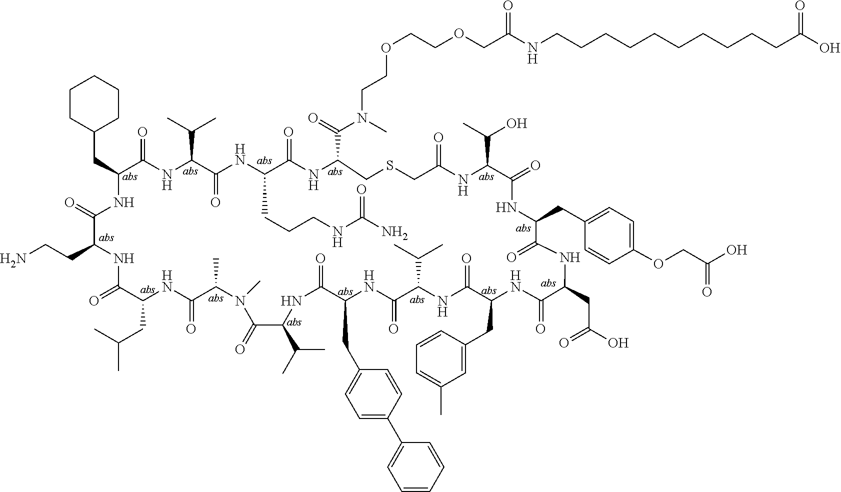 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2359 was prepared on a 50 μmol scale. The yield of the product was 9 mg, and its estimated purity by LCMS analysis was 98.9%. Analysis condition B: Retention time=1.94 min; ESI-MS(+) m/z [M+2H]²⁺: 1116.2.

### Preparation of Example 2360

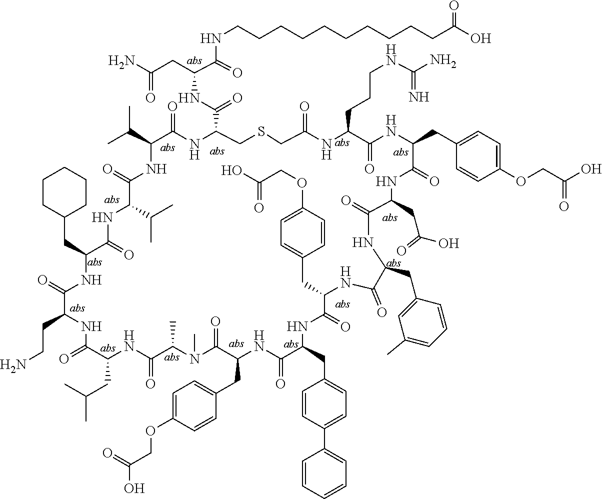 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2360 was prepared on a 50 μmol scale. The yield of the product was 53.5 mg, and its estimated purity by LCMS analysis was 96%. Analysis condition B: Retention time=1.82 min; ESI-MS(+) m/z [M+2H]²⁺: 1214.8.

### Preparation of Example 2361

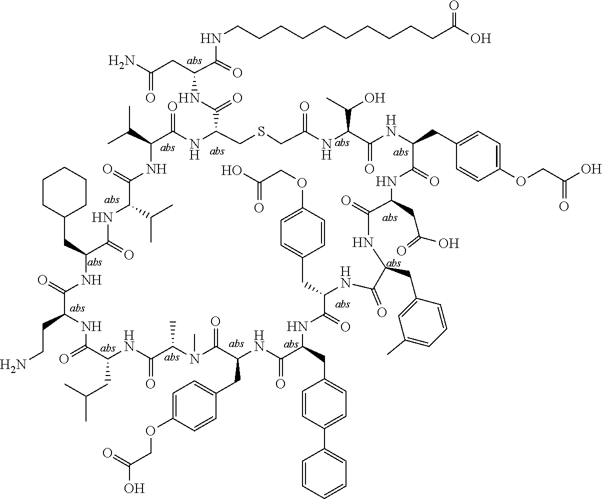 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2361 was prepared on a 50 μmol scale. The yield of the product was 89 mg, and its estimated purity by LCMS analysis was 94.6%. Analysis condition A: Retention time=1.44, 1.51 min; ESI-MS(+) m/z [M+2H]²⁺: 1186.95, 1187.1.

### Preparation of Example 2362

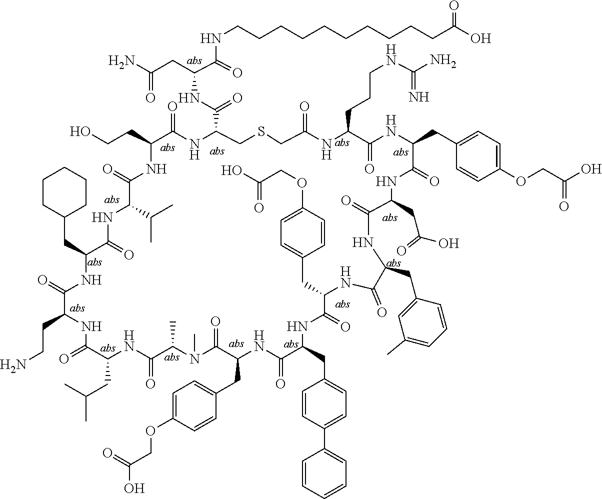 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2362 was prepared on a 50 μmol scale. The yield of the product was 27 mg, and its estimated purity by LCMS analysis was 96.3%. Analysis condition A: Retention time=1.48 min; ESI-MS(+) m/z [M+2H]²⁺: 1215.3.

### Preparation of Example 2363

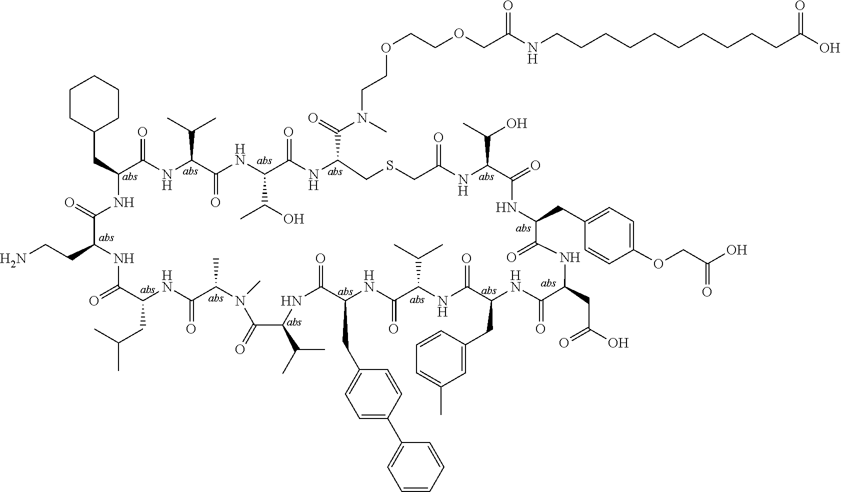 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2363 was prepared on a 50 μmol scale. The yield of the product was 22.8 mg, and its estimated purity by LCMS analysis was 95.7%. Analysis condition B: Retention time=1.87 min; ESI-MS(+) m/z [M+2H]²⁺: 1089.

### Preparation of Example 2364

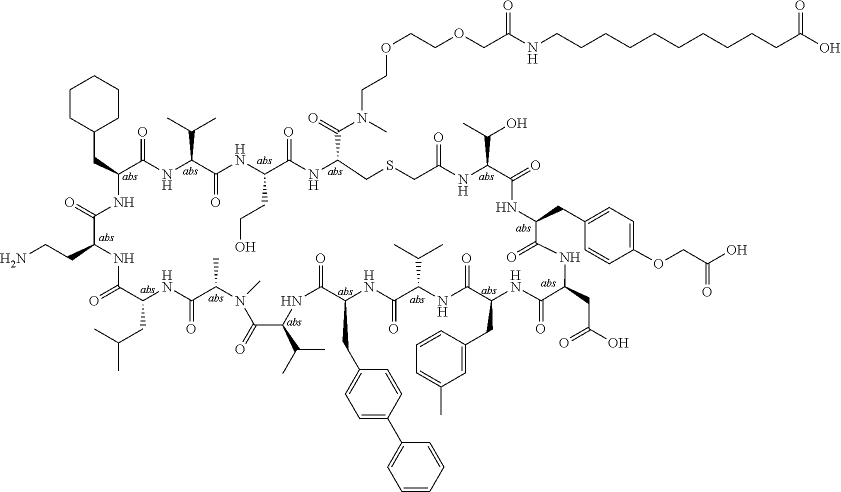 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2364 was prepared on a 50 μmol scale. The yield of the product was 19.5 mg, and its estimated purity by LCMS analysis was 95.2%. Analysis condition B: Retention time=1.85 min; ESI-MS(+) m/z [M+2H]²⁺: 1088.9.

### Preparation of Example 2365

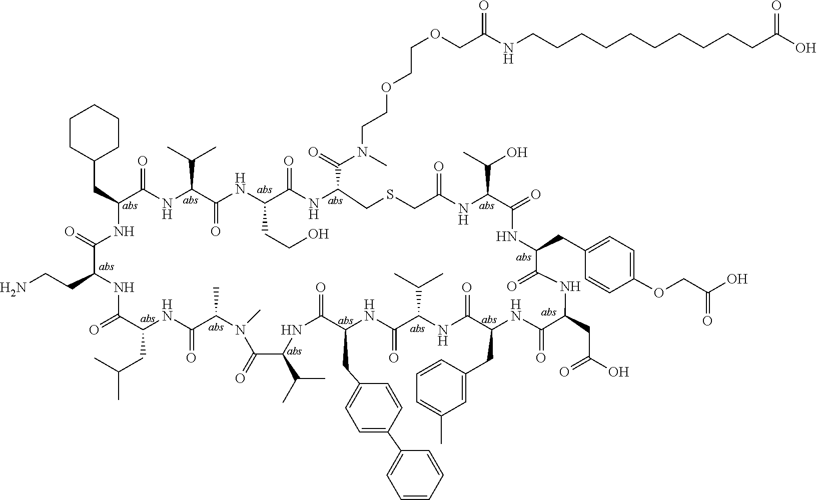 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2365 was prepared on a 50 μmol scale. The yield of the product was 6.9 mg, and its estimated purity by LCMS analysis was 97.4%. Analysis condition A: Retention time=1.76 min; ESI-MS(+) m/z [M+2H]²⁺: 1116.1.

### Preparation of Example 2366

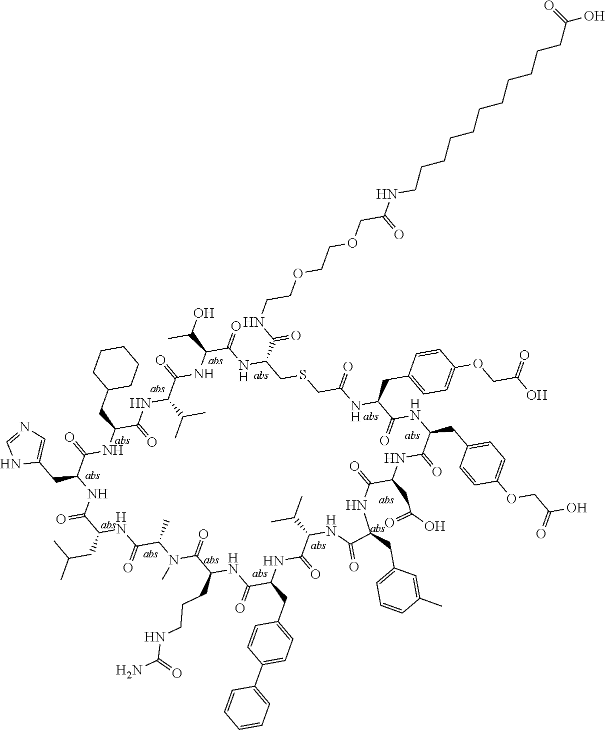 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCCOCCOCC(=O)NCCCCCCCCCCCC(=O)O)NC(=O)[C@H](C(C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2366 was prepared on a 50 μmol scale. The yield of the product was 19.8 mg, and its estimated purity by LCMS analysis was 100%. Analysis condition A: Retention time=1.64 min; ESI-MS(+) m/z [M+2H]²⁺: 1180.2.

### Preparation of Example 2367

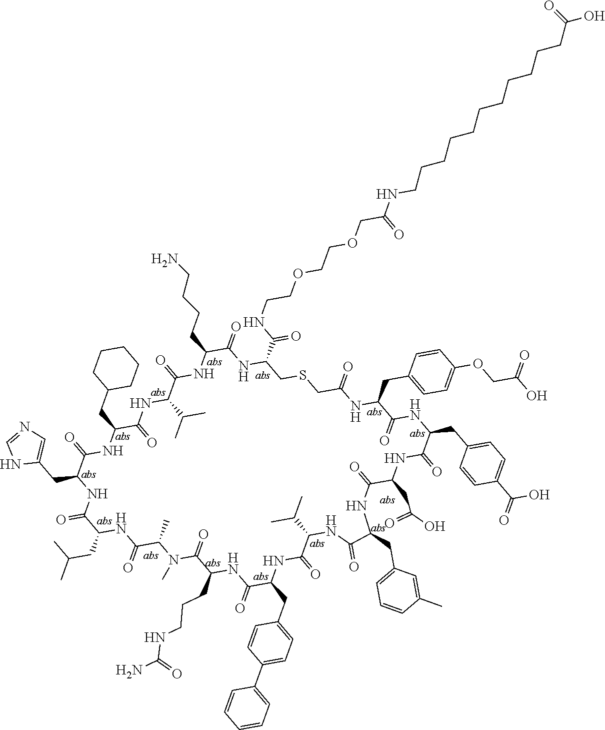 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCCC(=O)O)NC(=O)[C@H](CCCCN)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2367 was prepared on a 50 μmol scale. The yield of the product was 63.9 mg, and its estimated purity by LCMS analysis was 98.9%. Analysis condition A: Retention time=1.64 min; ESI-MS(+) m/z [M+2H]²⁺: 1194.1.

### Preparation of Example 2368

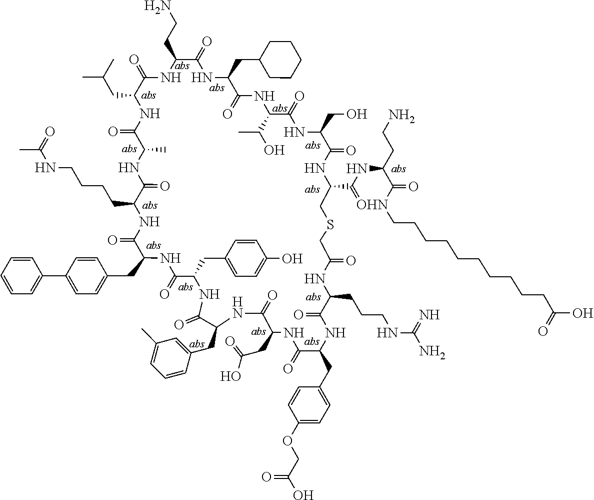 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](C)NC1=O]

Example 2368 was prepared on a 50 μmol scale. The yield of the product was 13.4 mg, and its estimated purity by LCMS analysis was 97.7%. Analysis condition A: Retention time=1.66 min; ESI-MS(+) m/z [M+2H]²⁺: 1148.3.

### Preparation of Example 2369

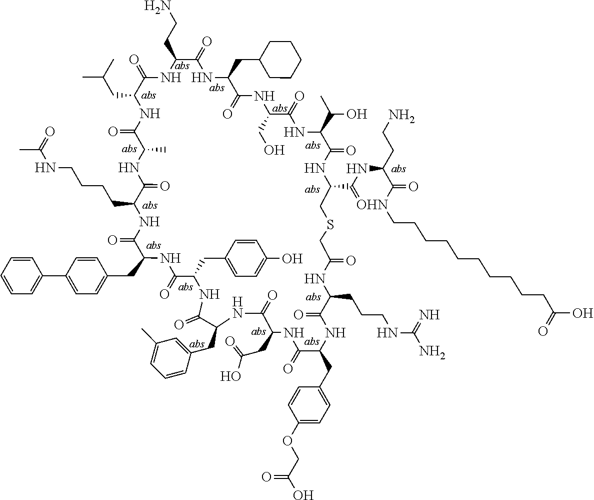 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](CO)NC(=O)[C@H](CC2CCCCC2)NC(=O)[C@H](CCN)NC(=O)[C@@H](CC(C)C)NC(=O)[C@H](C)NC1=O]

Example 2369 was prepared on a 50 μmol scale. The yield of the product was 17.4 mg, and its estimated purity by LCMS analysis was 99%. Analysis condition A: Retention time=1.72 min; ESI-MS(+) m/z [M+2H]²⁺: 1148.4.

### Preparation of Example 2370

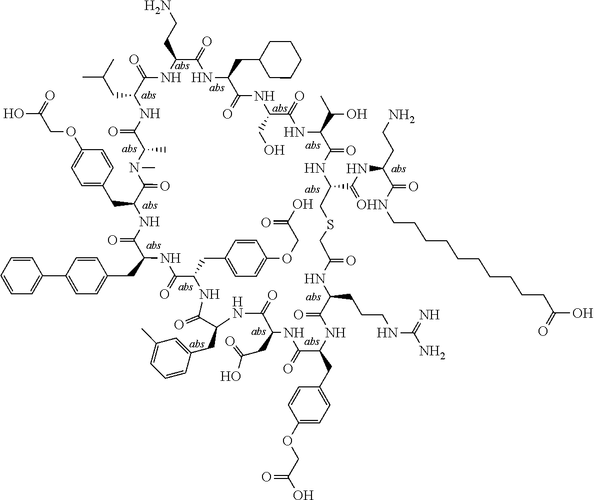 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2370 was prepared on a 50 μmol scale. The yield of the product was 25.8 mg, and its estimated purity by LCMS analysis was 97.2%. Analysis condition B: Retention time=1.71 min; ESI-MS(+) m/z [M+3H]³⁺: 802.3.

### Preparation of Example 2371

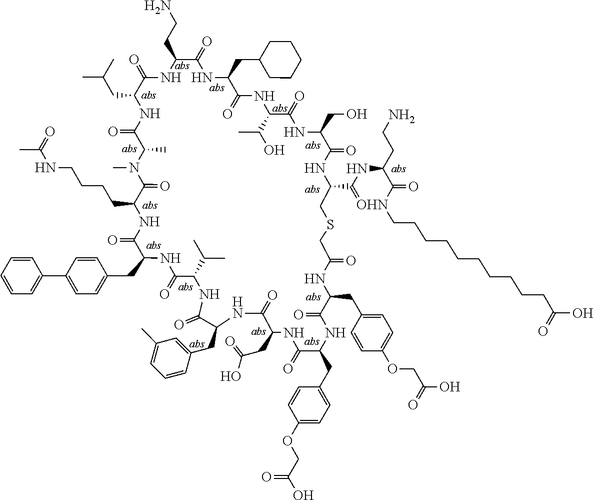 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](C)N(C)C1=O]

Example 2371 was prepared on a 50 μmol scale. The yield of the product was 1.4 mg, and its estimated purity by LCMS analysis was 96.1%. Analysis condition A: Retention time=1.56 min; ESI-MS(+) m/z [M+2H]²⁺: 1148.2.

### Preparation of Example 2372

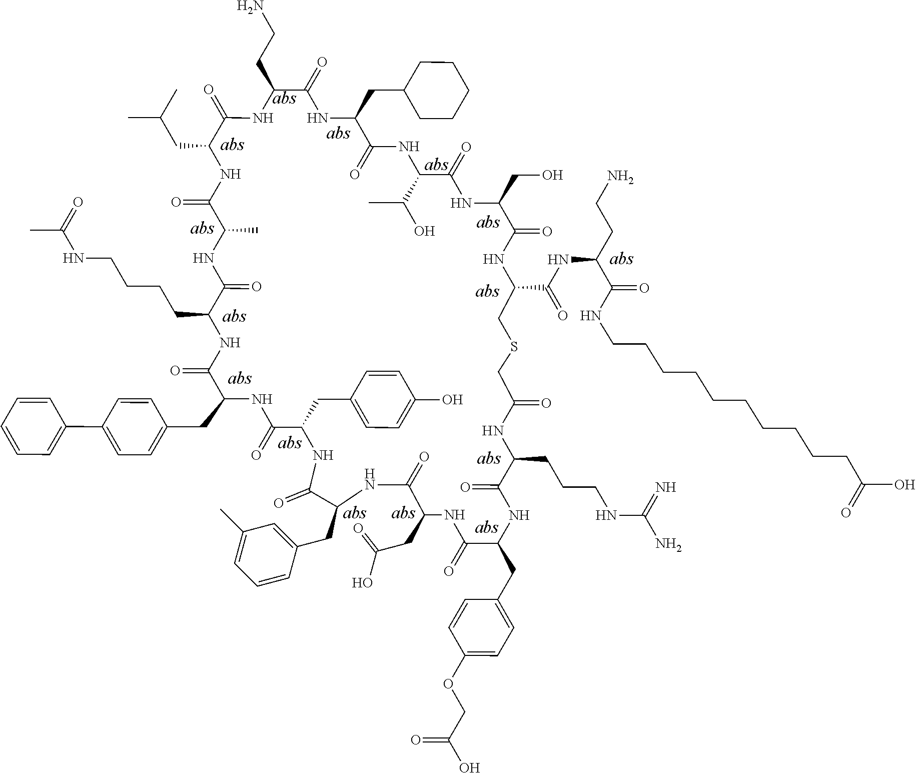 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](C)NC1=O]

Example 2372 was prepared on a 50 μmol scale. The yield of the product was 9.6 mg, and its estimated purity by LCMS analysis was 97.1%. Analysis condition A: Retention time=1.52 min; ESI-MS(+) m/z [M+2H]²⁺: 1177.

### Preparation of Example 2373

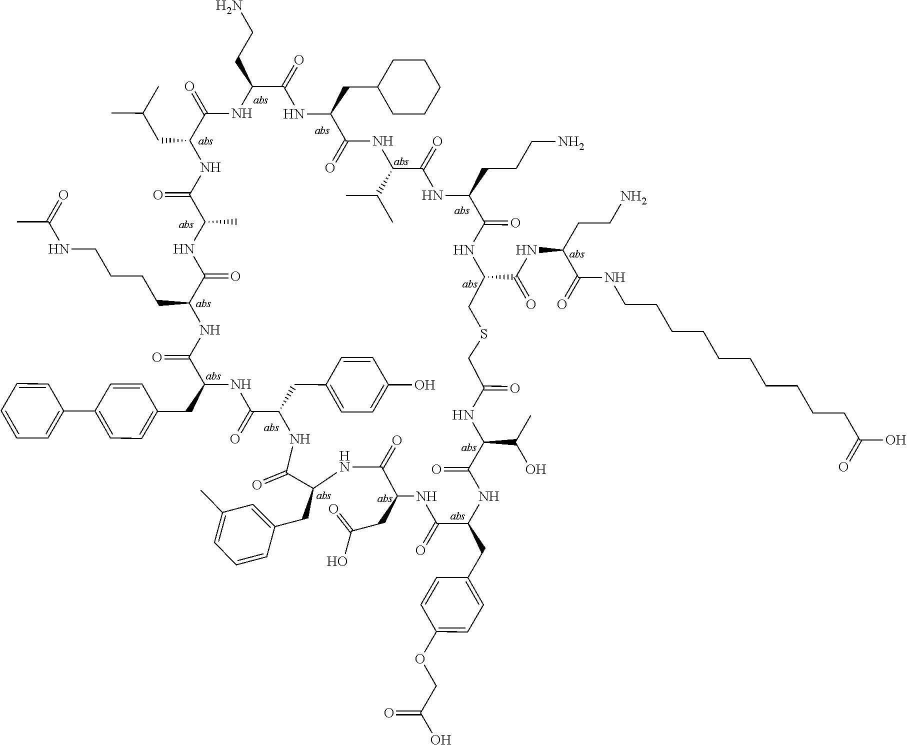 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)NC1=O]

Example 2373 was prepared on a 50 μmol scale. The yield of the product was 1.1 mg, and its estimated purity by LCMS analysis was 100%. Analysis condition B: Retention time=1.7 min; ESI-MS(+) m/z [M+2H]²⁺: 1139.6.

### Preparation of Example 2374

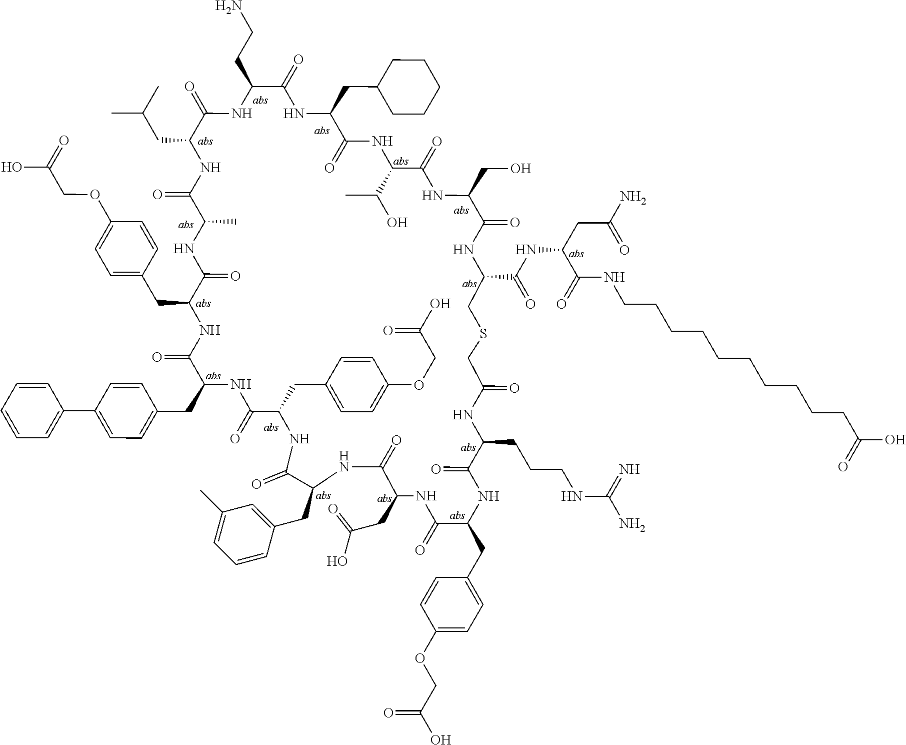 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](C)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2374 was prepared on a 50 μmol scale. The yield of the product was 63.1 mg, and its estimated purity by LCMS analysis was 91.9%. Analysis condition A: Retention time=1.49 min; ESI-MS(+) m/z [M+2H]²⁺: 1216.9.

### Preparation of Example 2375

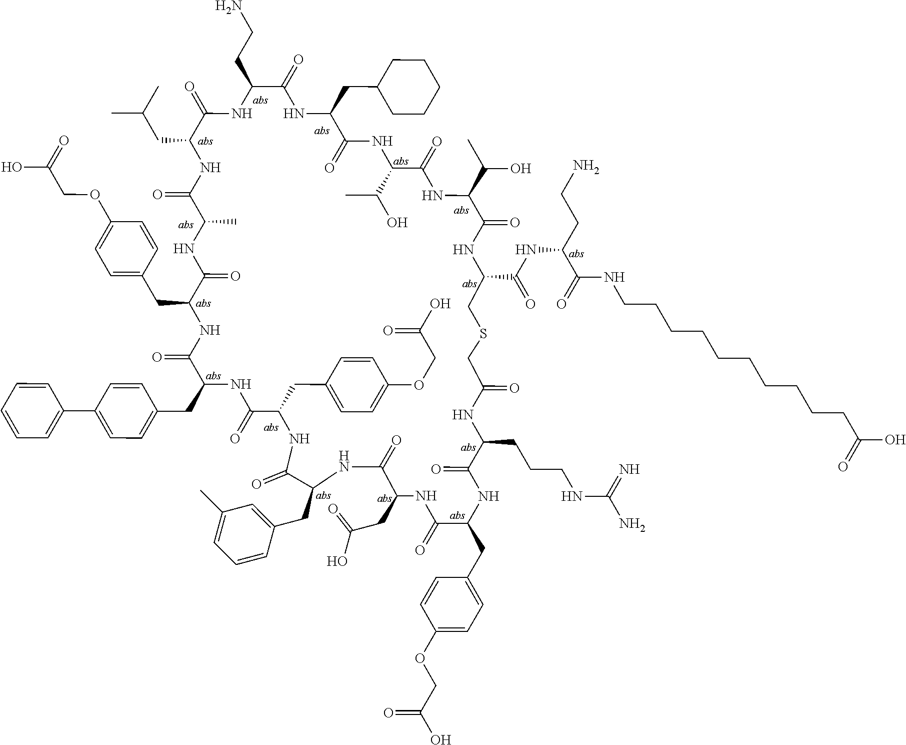 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CCN)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](C)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2375 was prepared on a 50 μmol scale. The yield of the product was 3.2 mg, and its estimated purity by LCMS analysis was 95.9%. Analysis condition: Retention time=1.66 min; ESI-MS(+) m/z [M+3H]³⁺: 811.2.

### Preparation of Example 2376

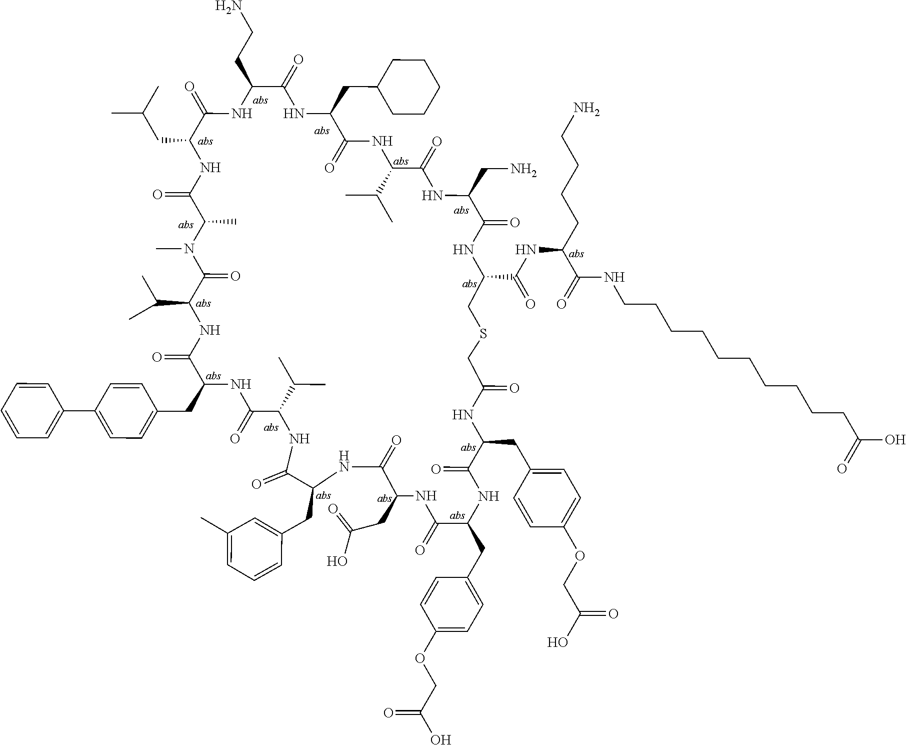 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2376 was prepared on a 50 μmol scale. The yield of the product was 51.6 mg, and its estimated purity by LCMS analysis was 95.1%. Analysis condition: Retention time=1.81 min; ESI-MS(+) m/z [M+3H]³⁺: 751.1.

### Preparation of Example 2377

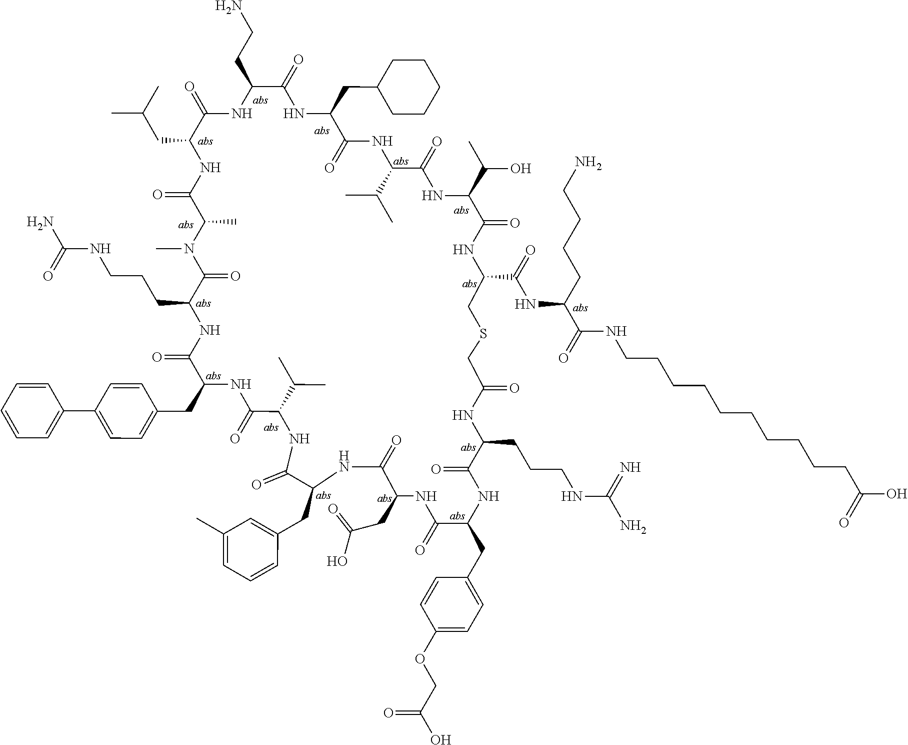 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2377 was prepared on a 50 μmol scale. The yield of the product was 52.1 mg, and its estimated purity by LCMS analysis was 97.2%. Analysis condition: Retention time=1.64 min; ESI-MS(+) m/z [M+3H]³⁺: 748.2.

### Preparation of Example 2378

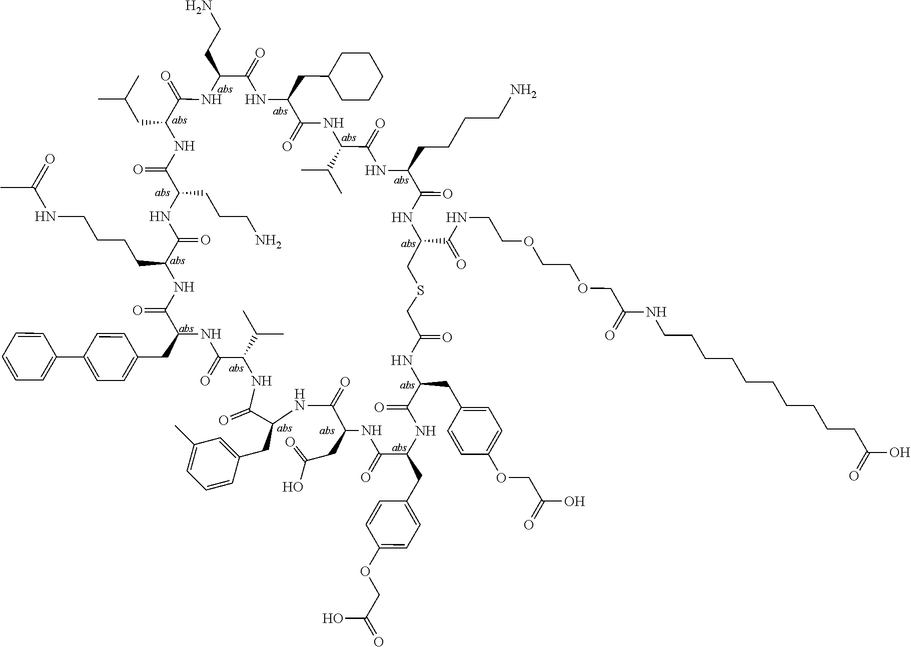 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Example 2378 was prepared on a 50 μmol scale. The yield of the product was 40.7 mg, and its estimated purity by LCMS analysis was 98.7%. Analysis condition B: Retention time=1.8 min; ESI-MS(+) m/z [M+2H]²⁺: 1223.1.

### Preparation of Example 2379

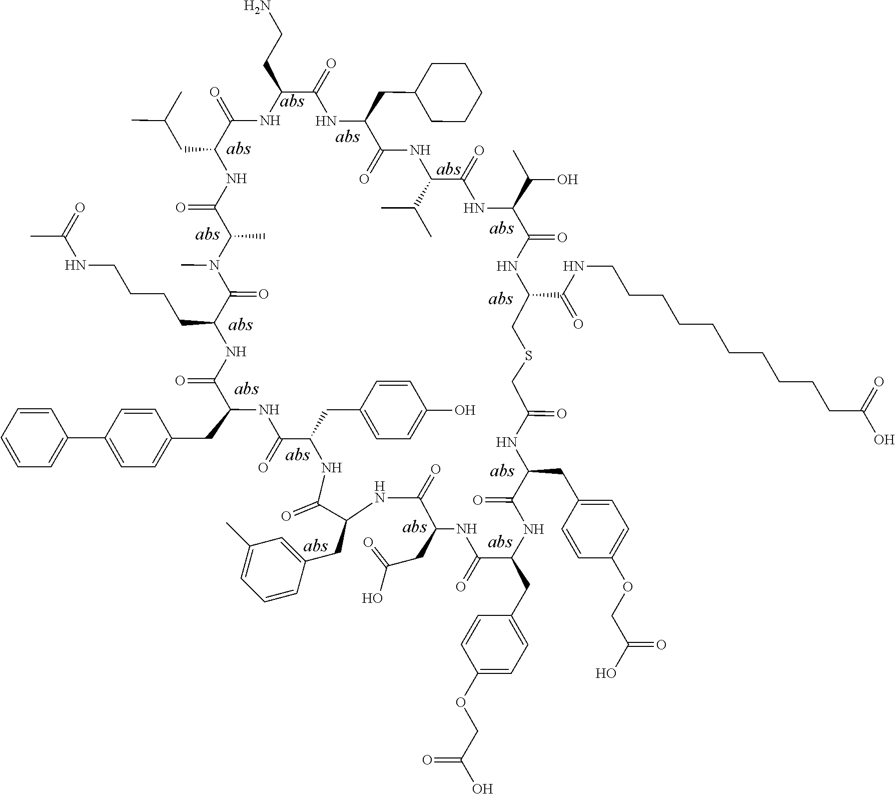 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2379 was prepared on a 50 μmol scale. The yield of the product was 55 mg, and its estimated purity by LCMS analysis was 100%. Analysis condition B: Retention time=1.99 min; ESI-MS(+) m/z [M+2H]²⁺: 1136.2.

### Preparation of Example 2380

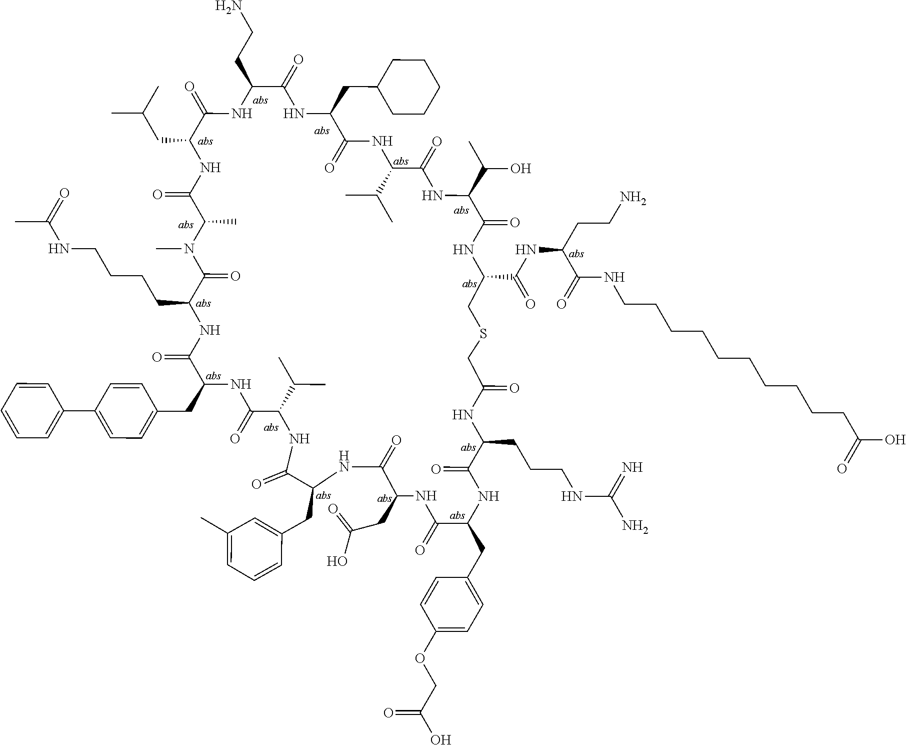 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2380 was prepared on a 50 μmol scale. The yield of the product was 20 mg, and its estimated purity by LCMS analysis was 100%. Analysis condition A: Retention time=1.57 min; ESI-MS(+) m/z [M+2H]²⁺: 1122.2.

### Preparation of Example 2381

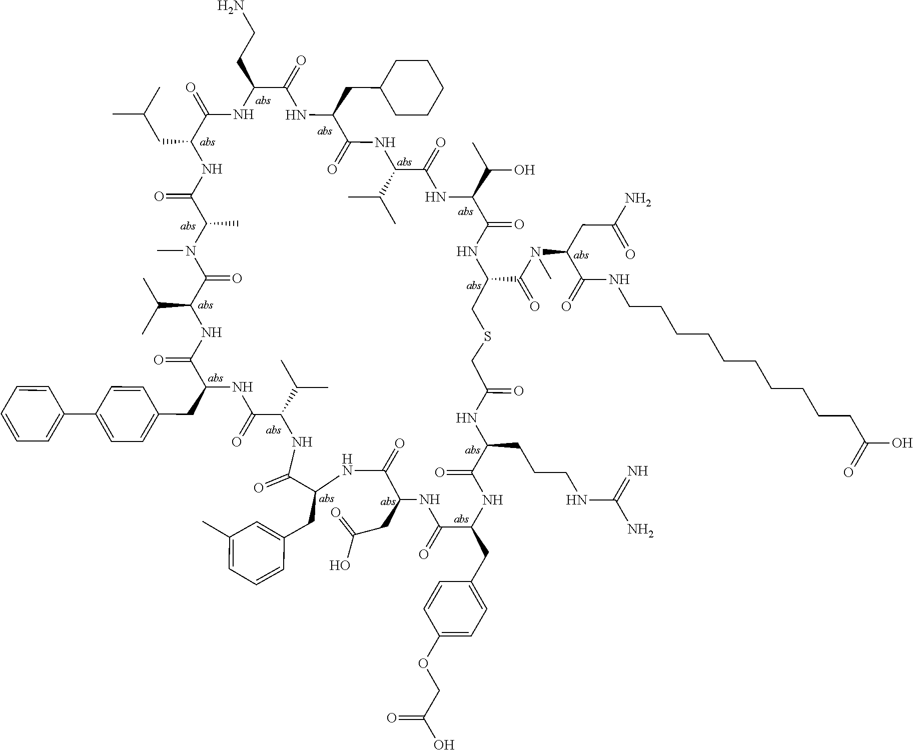 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)[C@@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2381 was prepared on a 50 μmol scale. The yield of the product was 32.8 mg, and its estimated purity by LCMS analysis was 100%. Analysis condition A: Retention time=1.74 min; ESI-MS(+) m/z [M+2H]²⁺: 1101.2.

### Preparation of Example 2382

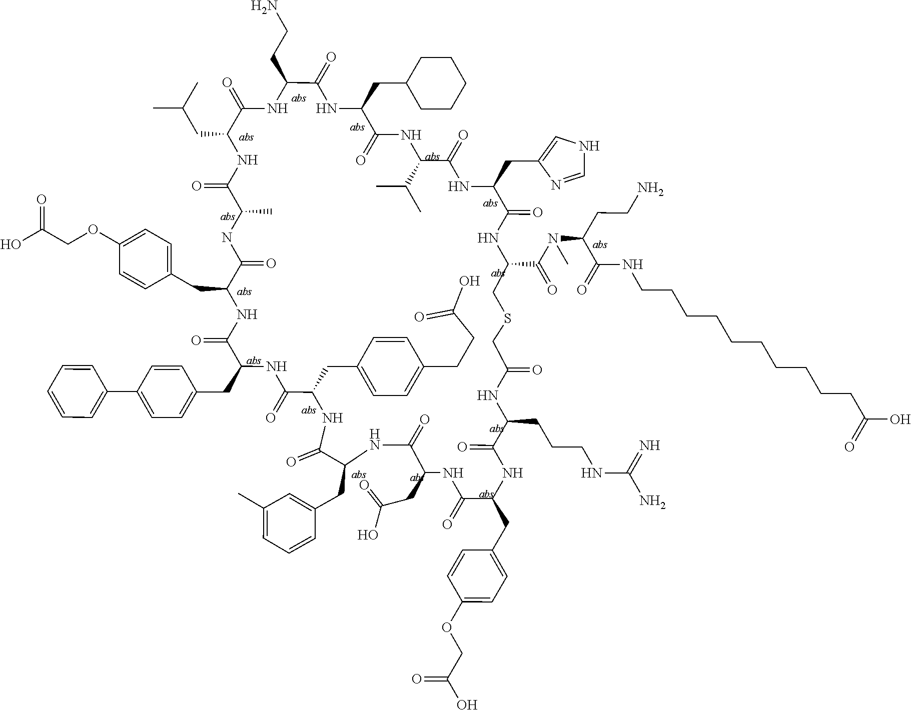 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)[C@@H](CCN)C(=O)NCCCCCCCCCCC(=O)O)NC(=O)[C@H](Cc3c[nH]cn3)NC(=O)[C@H](C(C)C)NC(=O)[C@H](CC3CCCCC3)NC(=O)[C@H](CCN)NC(=O)[C@@H](CC(C)C)NC(=O)[C@H](C)NC(=O)[C@H](Cc3ccc(OCC(=O)O)cc3)NC(=O)[C@H](Cc3ccc(-c4ccccc4)cc3)NC(=O)[C@H](Cc3ccc(CCC(=O)O)cc3)NC2=O)c1]

Example 2382 was prepared on a 50 μmol scale. The yield of the product was 73 mg, and its estimated purity by LCMS analysis was 95.6%. Analysis condition A: Retention time=1.49 min; ESI-MS(+) m/z [M+3H]³⁺: 818.1.

### Preparation of Example 2383

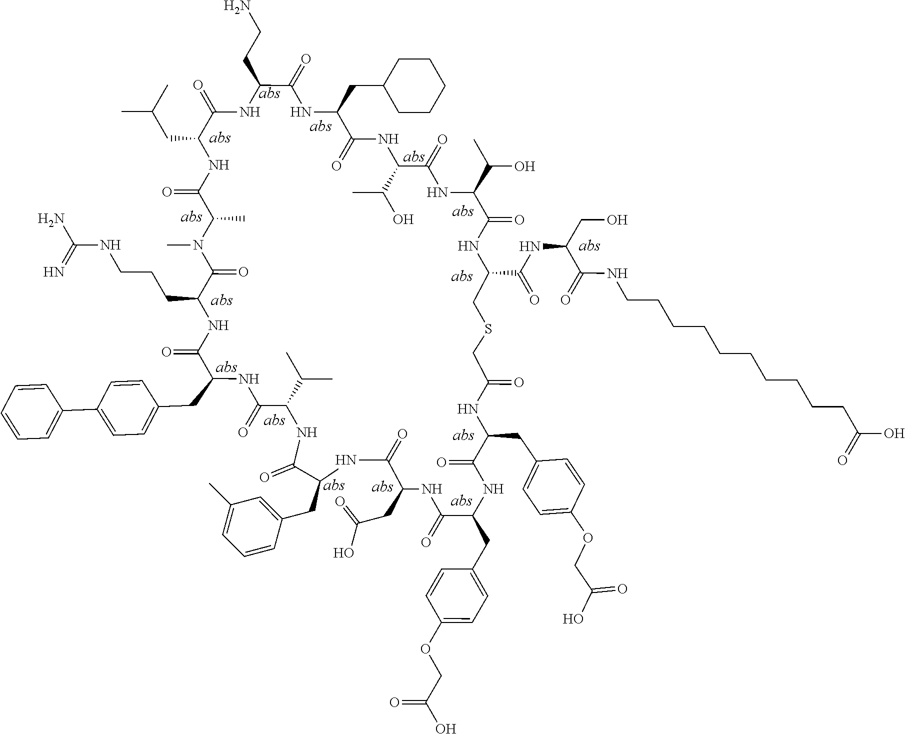 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CO)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](C(C)C)NC2=O)c1]

Example 2383 was prepared on a 50 μmol scale. The yield of the product was 21.2 mg, and its estimated purity by LCMS analysis was 100%. Analysis condition: Retention time=1.78 min; ESI-MS(+) m/z [M+2H]²⁺: 1141.1.

### Preparation of Example 2384

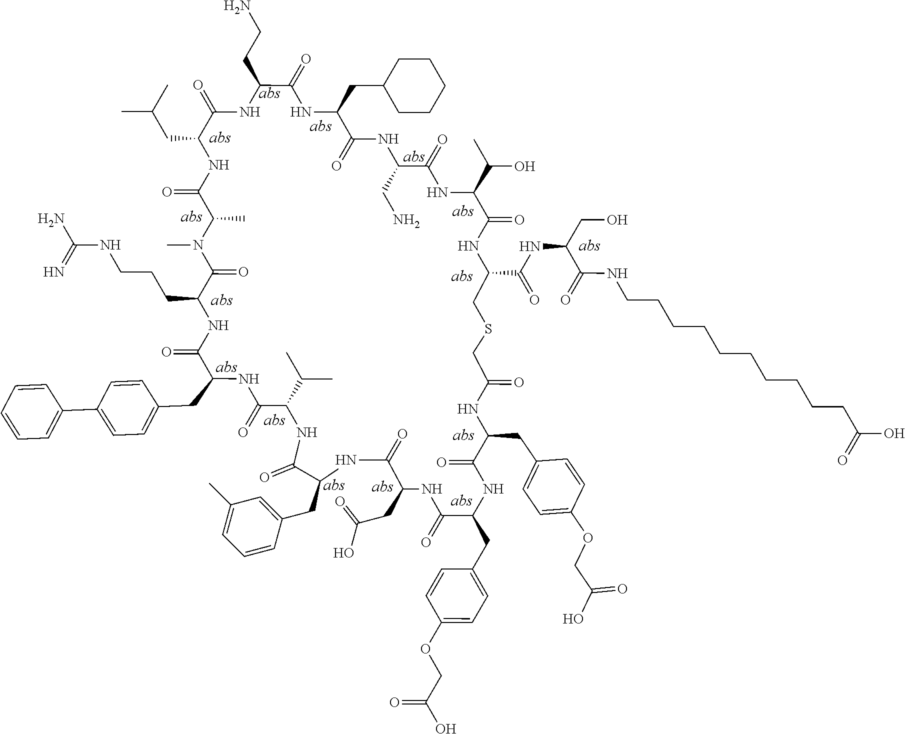 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CO)C(=O)NCCCCCCCCCCC(=O)O)NC(=O)[C@H](C(C)O)NC(=O)[C@H](CN)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](C(C)C)NC2=O)c1]

Example 2384 was prepared on a 50 μmol scale. The yield of the product was 9 mg, and its estimated purity by LCMS analysis was 96.3%. Analysis condition B: Retention time=1.74 min; ESI-MS(+) m/z [M+3H]³⁺: 756.9.

### Preparation of Example 2385

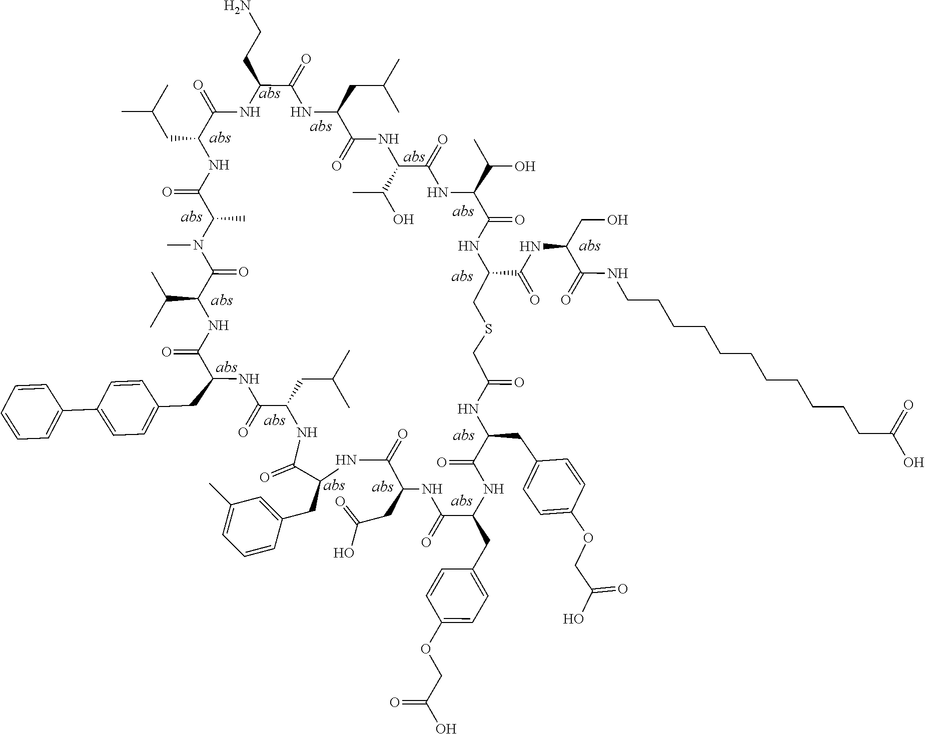 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CO)C(=O)NCCCCCCCCCCCC(=O)O)NC(=O)[C@H](C(C)O)NC(=O)[C@H](C(C)O)NC(=O)[C@H](CC(C)C)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2385 was prepared on a 50 μmol scale. The yield of the product was 8.1 mg, and its estimated purity by LCMS analysis was 100%. Analysis condition A: Retention time=1.61 min; ESI-MS(+) m/z [M+2H]²⁺: 1106.1.

### Preparation of Example 2386

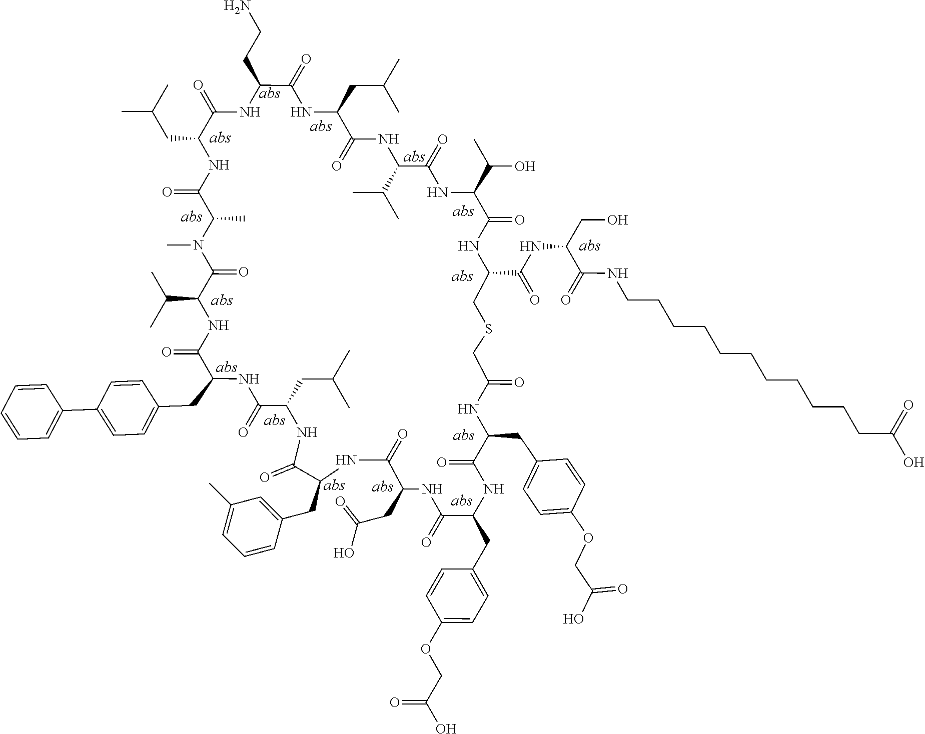 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@H](CO)C(=O)NCCCCCCCCCCCC(=O)O)NC(=O)[C@H](C(C)O)NC(=O)[C@H](C(C)C)NC(=O)[C@H](CC(C)C)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](CC(C)C)NC2=O)c1]

Example 2386 was prepared on a 50 μmol scale. The yield of the product was 4.8 mg, and its estimated purity by LCMS analysis was 95.7%. Analysis condition A: Retention time=1.5 min; ESI-MS(+) m/z [M+2H]²⁺: 1099.6.

### Preparation of Example 2387

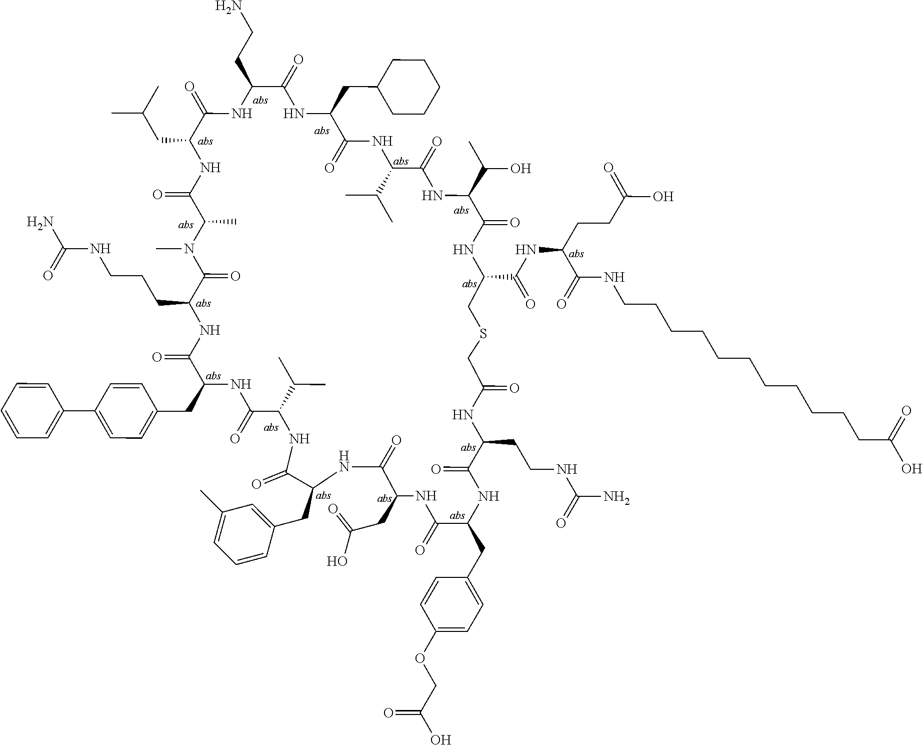 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(N)=O)NC(=O)CSC[C@@H](C(=O)N[C@@H](CCC(=O)O)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2387 was prepared on a 50 μmol scale. The yield of the product was 17.8 mg, and its estimated purity by LCMS analysis was 100%. Analysis condition A: Retention time=1.68 min; ESI-MS(+) m/z [M+2H]²⁺: 1144.1.

### Preparation of Example 2388

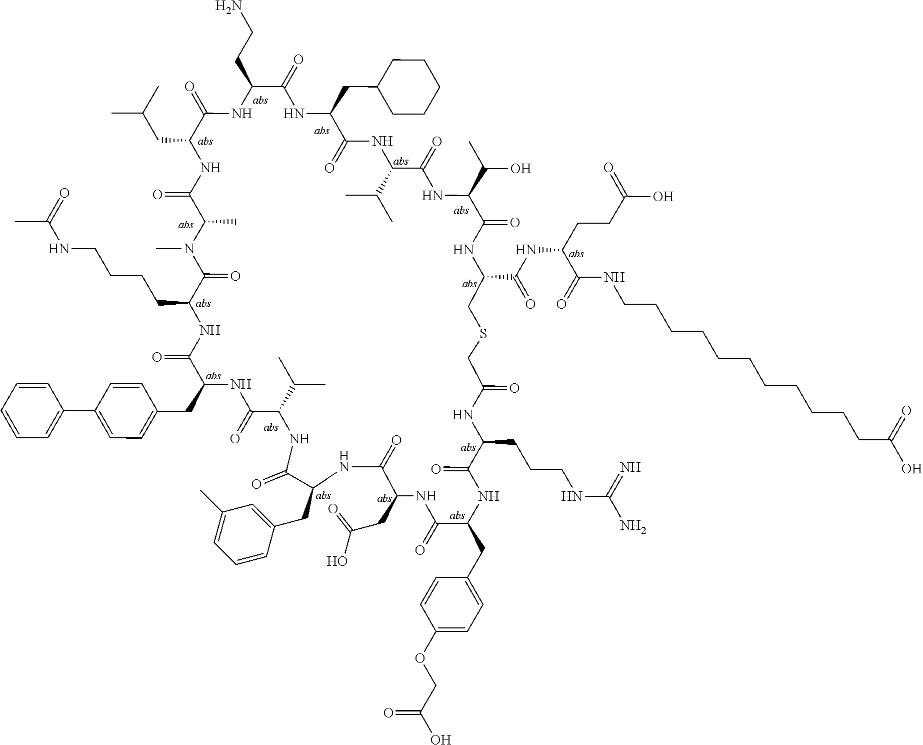 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CCC(=O)O)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2388 was prepared on a 50 μmol scale. The yield of the product was 40.3 mg, and its estimated purity by LCMS analysis was 96.5%. Analysis condition B: Retention time=1.9 min; ESI-MS(+) m/z [M+2H]²⁺: 1144.2.

### Preparation of Example 2389

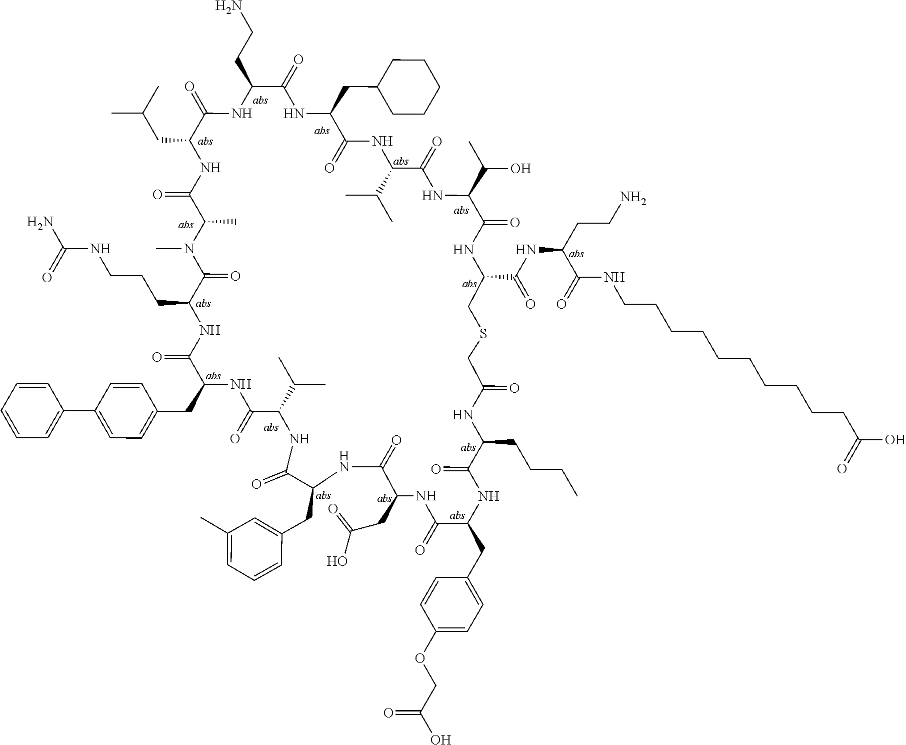 [SMILES: CCCC[C@@H]1NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Example 2389 was prepared on a 50 μmol scale. The yield of the product was 23.4 mg, and its estimated purity by LCMS analysis was 100%. Analysis condition A: Retention time=1.84 min; ESI-MS(+) m/z [M+2H]²⁺: 1101.2.

### Preparation of Example 2390

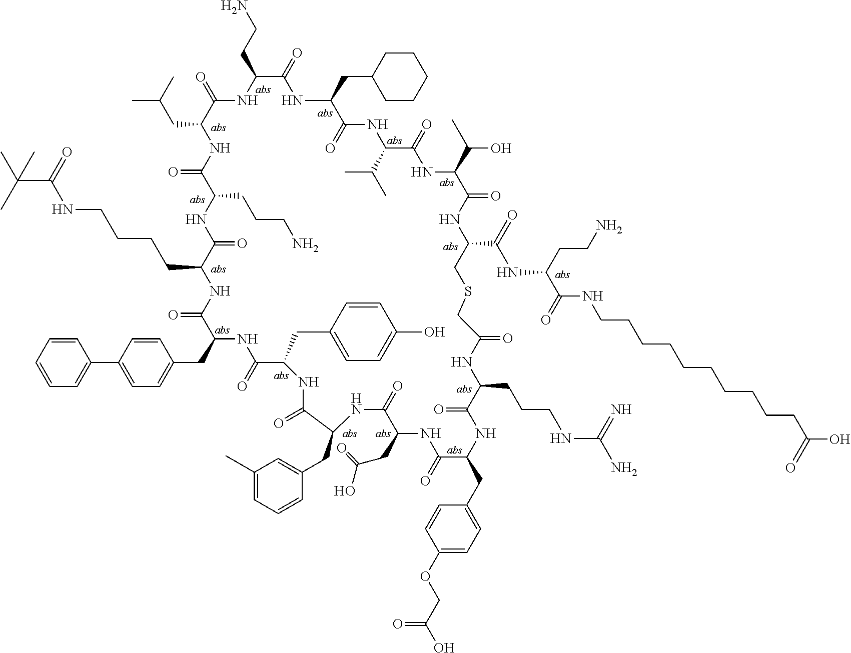 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCNC(=O)C(C)(C)C)NC(=O)[C@H](Cc3ccc(-c4ccccc4)cc3)NC(=O)[C@H](Cc3ccc(O)cc3)NC2=O)c1]

Example 2390 was prepared on a 50 μmol scale. The yield of the product was 34.7 mg, and its estimated purity by LCMS analysis was 96.3%. Analysis condition B: Retention time=1.79 min; ESI-MS(+) m/z [M+2H]²⁺: 1175.1.

### Preparation of Example 2391

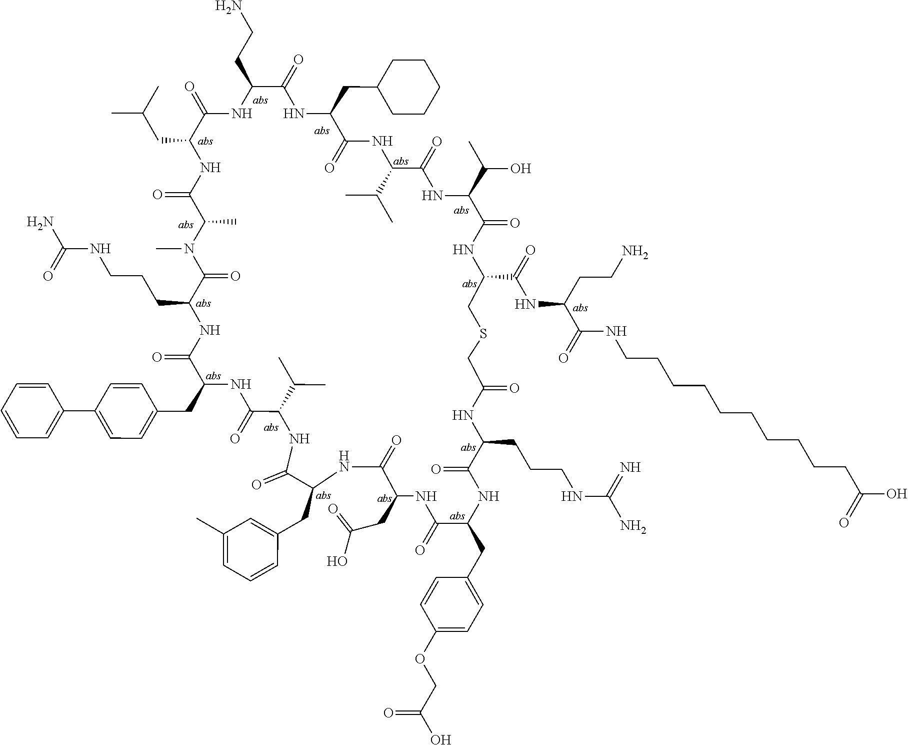 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2391 was prepared on a 50 μmol scale. The yield of the product was 48.1 mg, and its estimated purity by LCMS analysis was 95.6%. Analysis condition A: Retention time=1.69 min; ESI-MS(+) m/z [M+3H]³⁺: 749.

### Preparation of Example 2392

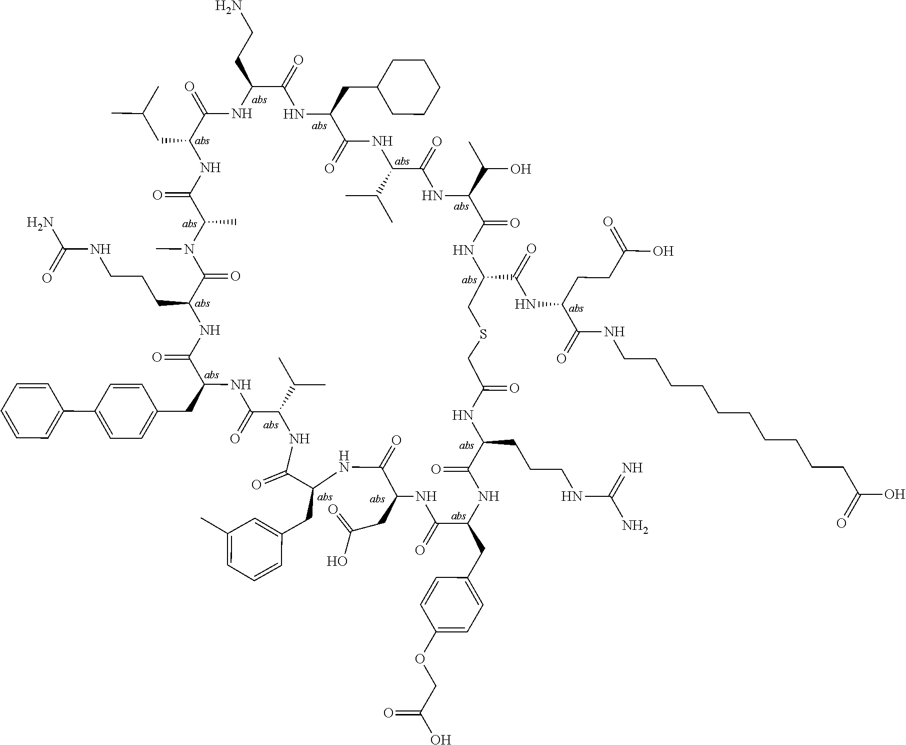 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CCC(=O)O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2392 was prepared on a 50 μmol scale. The yield of the product was 3.1 mg, and its estimated purity by LCMS analysis was 100%. Analysis condition A: Retention time=1.61 min; ESI-MS(+) m/z [M+2H]²⁺: 1130.

### Preparation of Example 2393

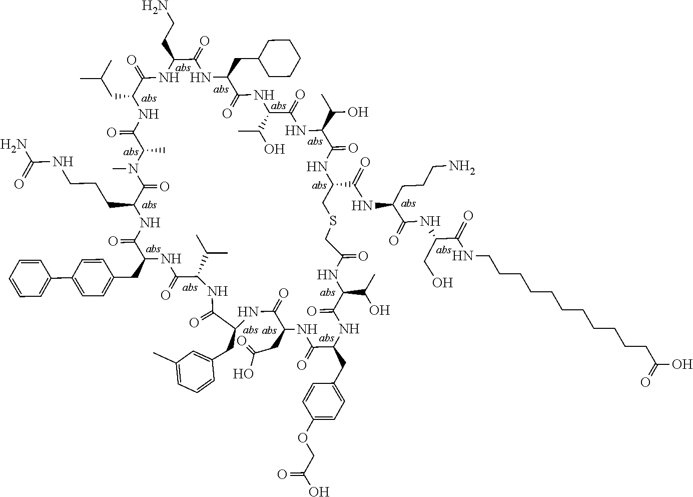 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2393 was prepared on a 50 μmol scale. The yield of the product was 2.8 mg, and its estimated purity by LCMS analysis was 100%. Analysis condition: Retention time=1.74 min; ESI-MS(+) m/z [M+2H]²⁺: 1139.1.

### Preparation of Example 2394

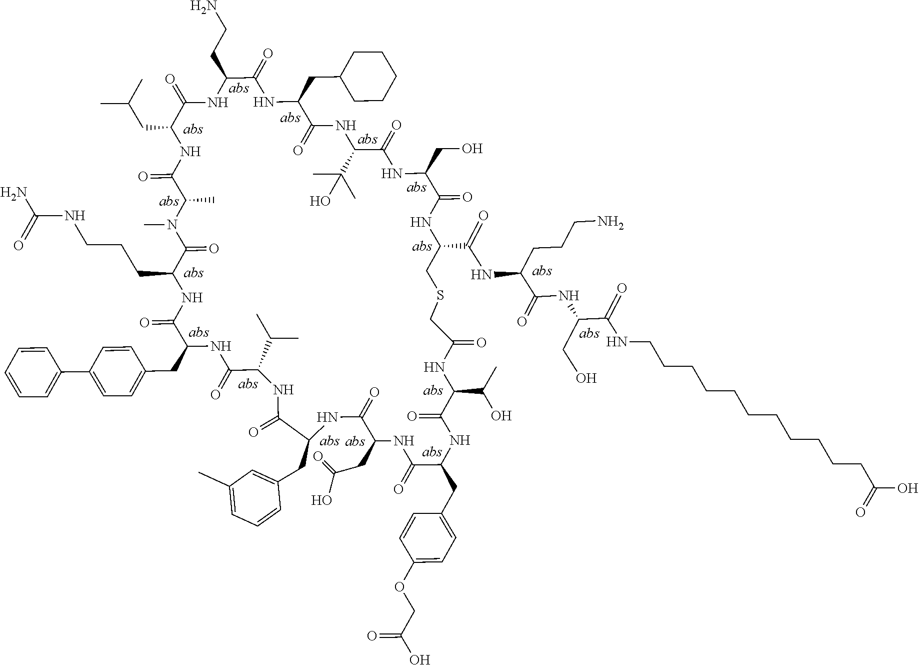 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2394 was prepared on a 50 μmol scale. The yield of the product was 12.9 mg, and its estimated purity by LCMS analysis was 95.3%. Analysis condition: Retention time=1.84 min; ESI-MS(+) m/z [M+2H]²⁺: 1146.4.

### Preparation of Example 2395

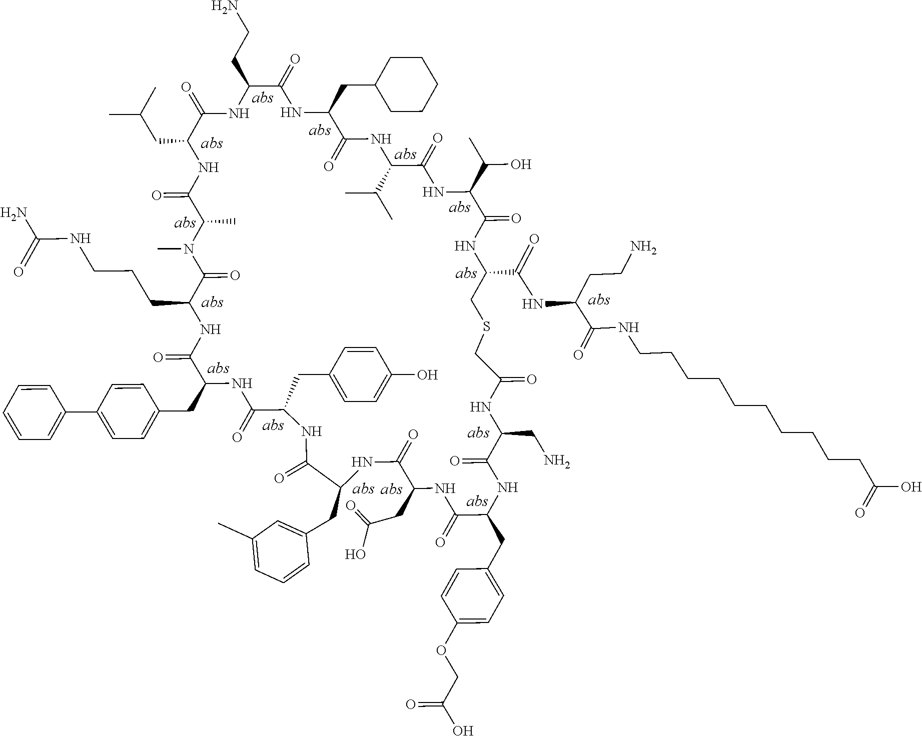 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(O)cc3)NC2=O)c1]

Example 2395 was prepared on a 50 μmol scale. The yield of the product was 20.2 mg, and its estimated purity by LCMS analysis was 95.600. Analysis condition: Retention time=1.68 min; ESI-MS(+) m/z [M+3H]³⁺: 770.2.

### Preparation of Example 2396

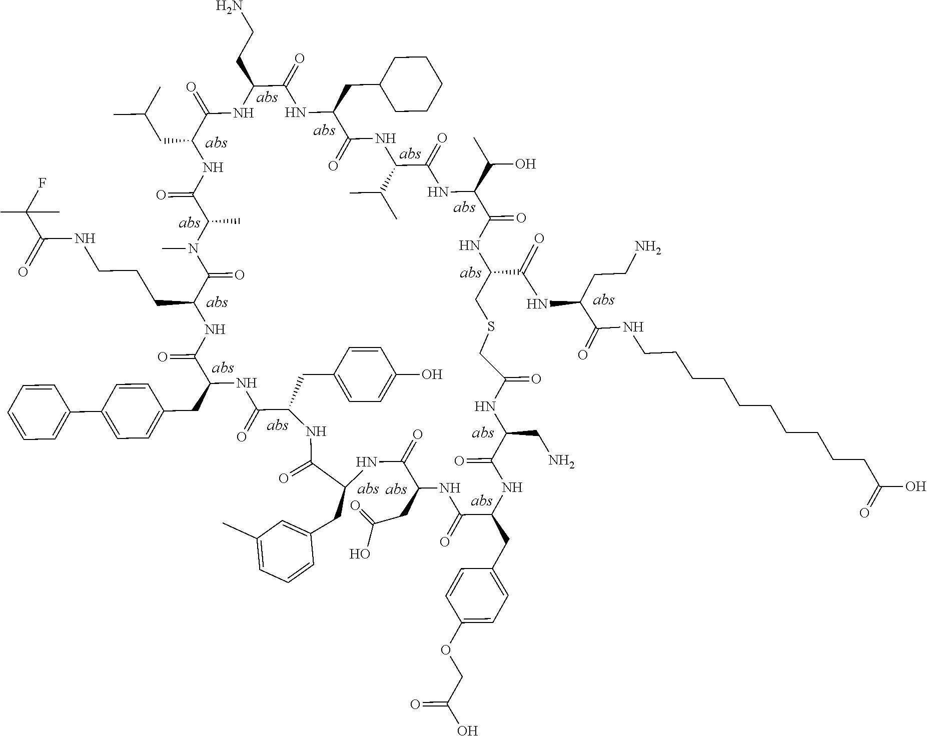 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=O)C(C)(C)F)NC(=O)[C@H](Cc3ccc(-c4ccccc4)cc3)NC(=O)[C@H](Cc3ccc(O)cc3)NC2=O)c1]

Example 2396 was prepared on a 50 μmol scale. The yield of the product was 8.4 mg, and its estimated purity by LCMS analysis was 96.7%. Analysis condition: Retention time=1.73 min; ESI-MS(+) m/z [M+2H]²⁺: 1177.2.

### Preparation of Example 2397

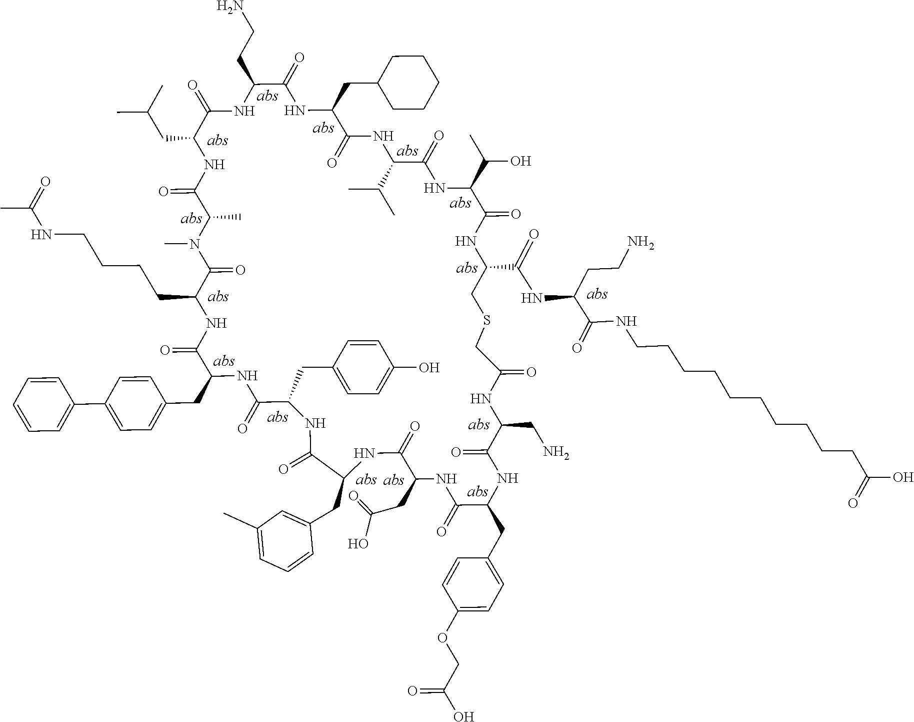 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2397 was prepared on a 50 μmol scale. The yield of the product was 38.1 mg, and its estimated purity by LCMS analysis was 96%. Analysis condition: Retention time=1.69 min; ESI-MS(+) m/z [M+2H]²⁺: 1161.

### Preparation of Example 2398

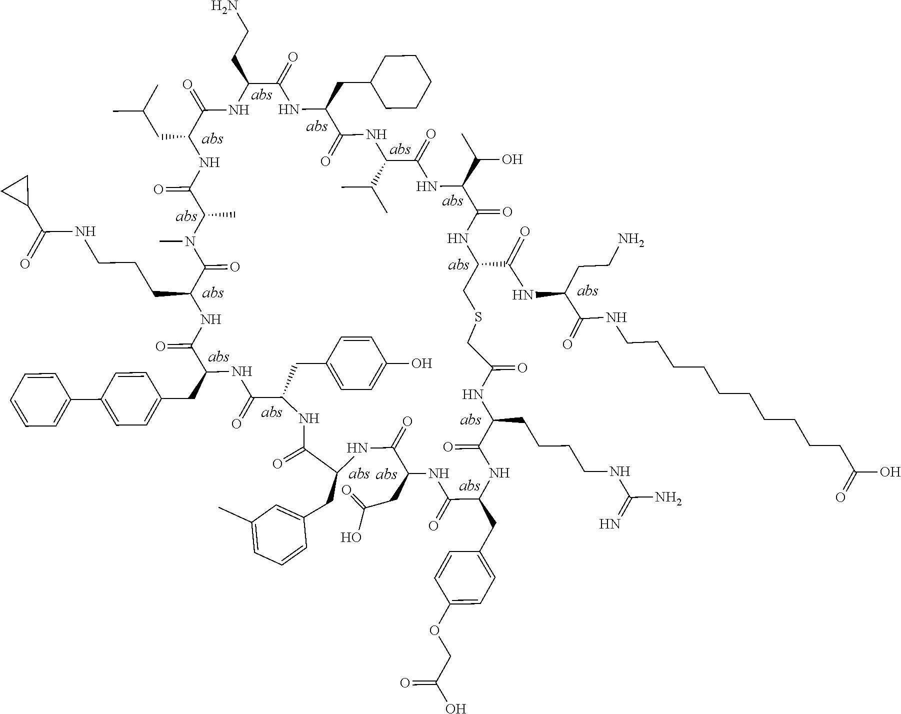 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=O)C3CC3)NC(=O)[C@H](Cc3ccc(-c4ccccc4)cc3)NC(=O)[C@H](Cc3ccc(O)cc3)NC2=O)c1]

Example 2398 was prepared on a 50 μmol scale. The yield of the product was 19.4 mg, and its estimated purity by LCMS analysis was 95.2%. Analysis condition: Retention time=1.71 min; ESI-MS(+) m/z [M+2H]²⁺: 1167.2.

### Preparation of Example 2399

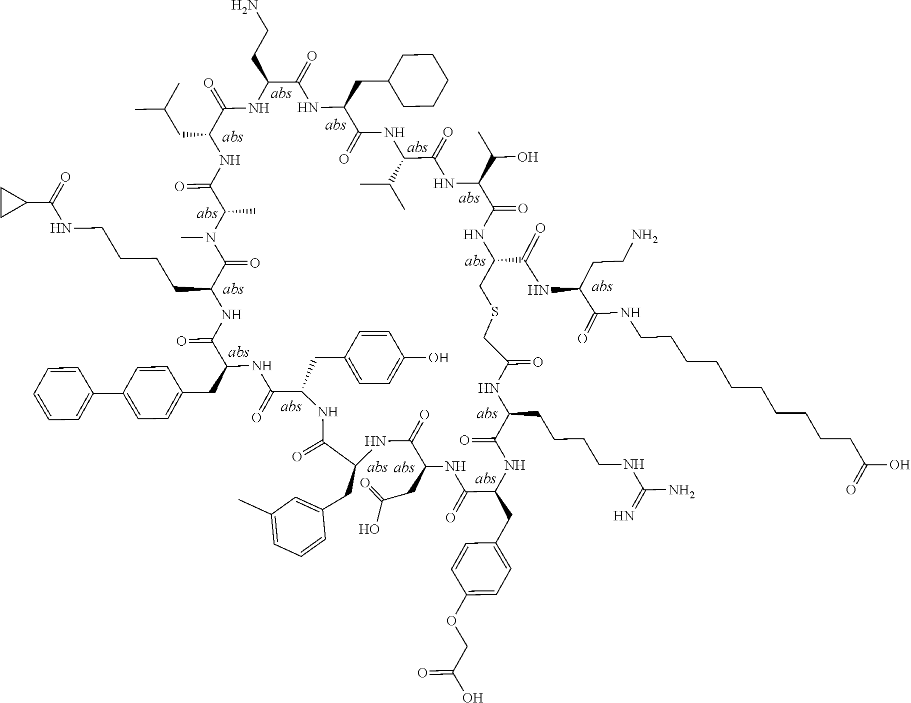 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCCNC(=O)C3CC3)NC(=O)[C@H](Cc3ccc(-c4ccccc4)cc3)NC(=O)[C@H](Cc3ccc(O)cc3)NC2=O)c1]

Example 2399 was prepared on a 50 μmol scale. The yield of the product was 16 mg, and its estimated purity by LCMS analysis was 95.2%. Analysis condition: Retention time=1.81 min; ESI-MS(+) m/z [M+3H]⁺: 783.2.

### Preparation of Example 2400

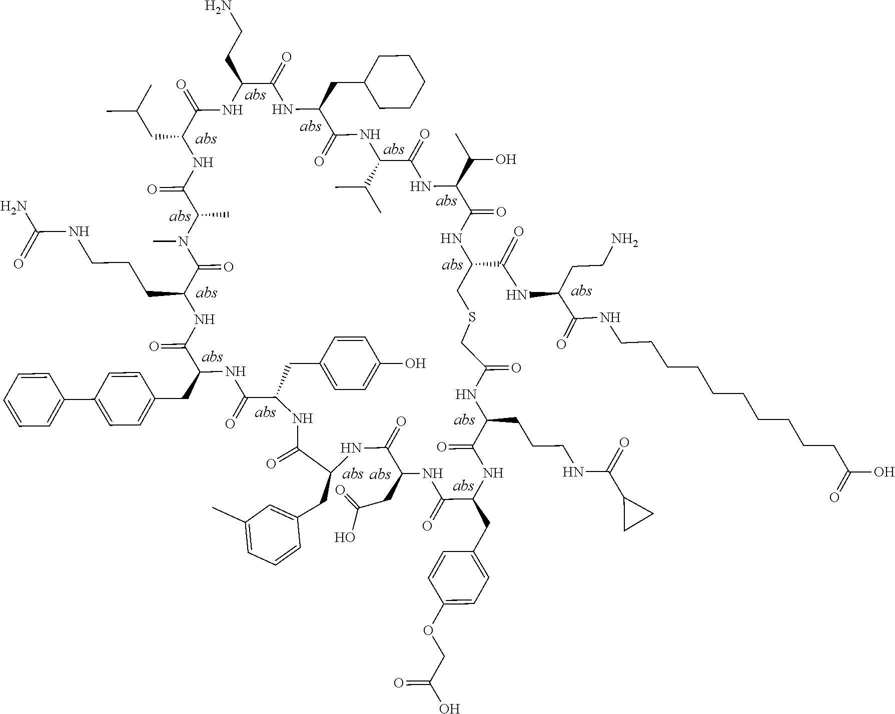 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=O)C3CC3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(O)cc3)NC2=O)c1]

Example 2400 was prepared on a 50 μmol scale. The yield of the product was 11.2 mg, and its estimated purity by LCMS analysis was 96.3%. Analysis condition A: Retention time=1.55 min; ESI-MS(+) m/z [M+2H]²⁺: 1160.

### Preparation of Example 2401

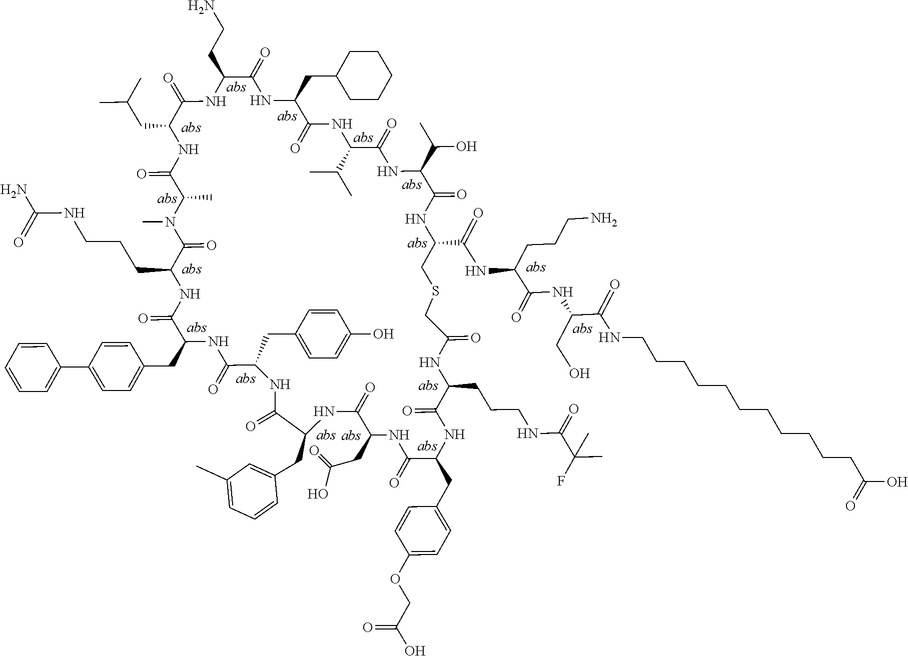 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=O)C(C)(C)F)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(O)cc3)NC2=O)c1]

Example 2401 was prepared on a 50 μmol scale. The yield of the product was 14.1 mg, and its estimated purity by LCMS analysis was 95.8%. Analysis condition: Retention time=1.76 min; ESI-MS(+) m/z [M+2H]²⁺: 1228.2.

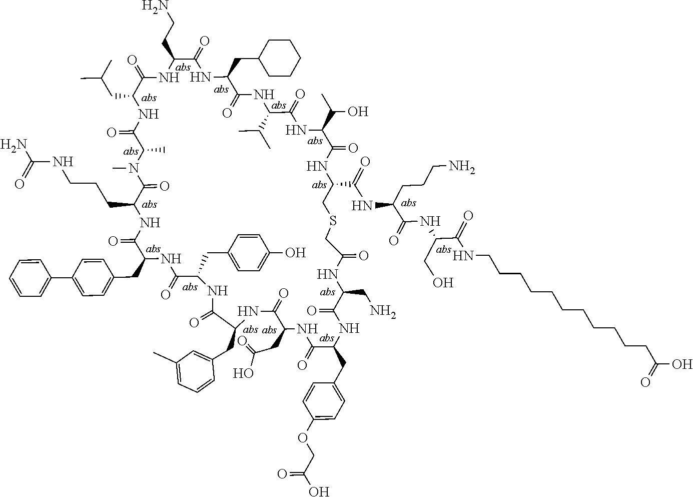 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](Cc3ccc(O)cc3)NC2=O)c1]

Example 2402 was prepared on a 50 μmol scale. The yield of the product was 8.4 mg, and its estimated purity by LCMS analysis was 95.9%. Analysis condition A: Retention time=1.6 min; ESI-MS(+) m/z [M+2H]²⁺: 1170.

### Preparation of Example 2403

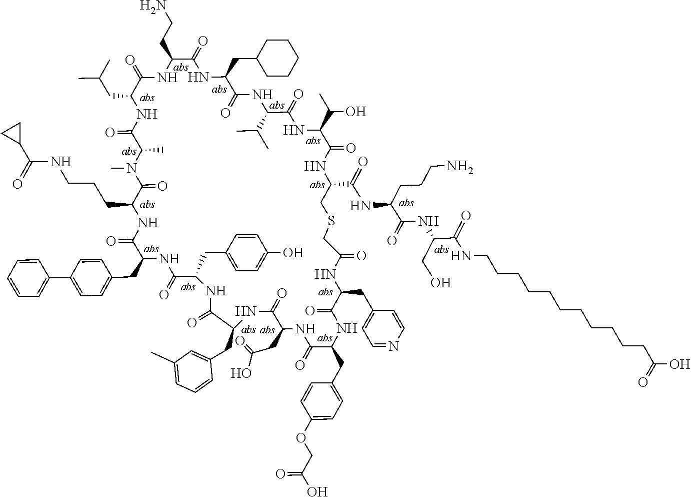 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=O)C3CC3)NC(=O)[C@H](Cc3ccc(-c4ccccc4)cc3)NC(=O)[C@H](Cc3ccc(O)cc3)NC2=O)c1]

Example 2403 was prepared on a 50 μmol scale. The yield of the product was 3.8 mg, and its estimated purity by LCMS analysis was 99.1%. Analysis condition: Retention time=1.76 min; ESI-MS(+) m/z [M+2H]²⁺: 1213.3.

### Preparation of Example 2404

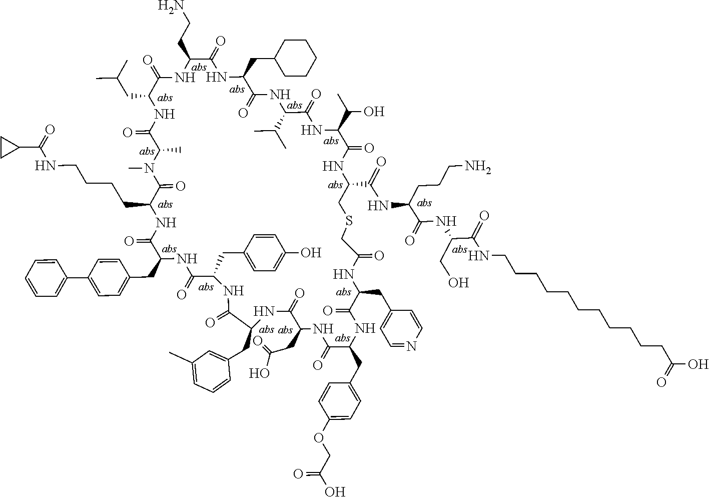 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCCNC(=O)C3CC3)NC(=O)[C@H](Cc3ccc(-c4ccccc4)cc3)NC(=O)[C@H](Cc3ccc(O)cc3)NC2=O)c1]

Example 2404 was prepared on a 50 μmol scale. The yield of the product was 18.4 mg, and its estimated purity by LCMS analysis was 98.2%. Analysis condition A: Retention time=1.72 min; ESI-MS(+) m/z [M+3H]³⁺: 814.3.

### Preparation of Example 2405

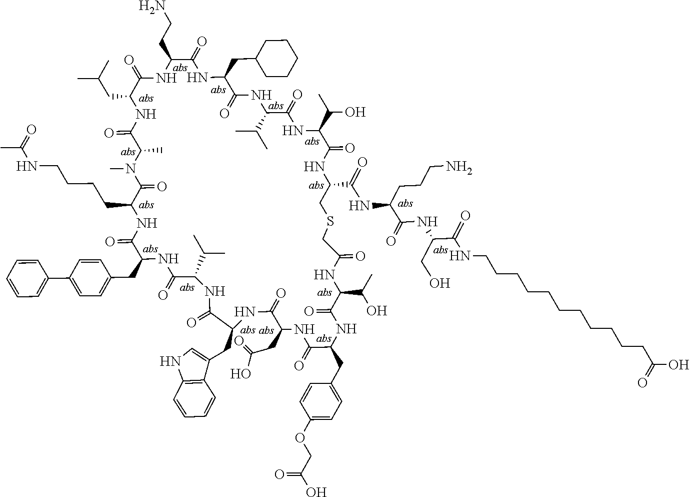 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2405 was prepared on a 50 μmol scale. The yield of the product was 19 mg, and its estimated purity by LCMS analysis was 98.2%. Analysis condition B: Retention time=1.82 min; ESI-MS(+) m/z [M+2H]²⁺: 1165.

### Preparation of Example 2406

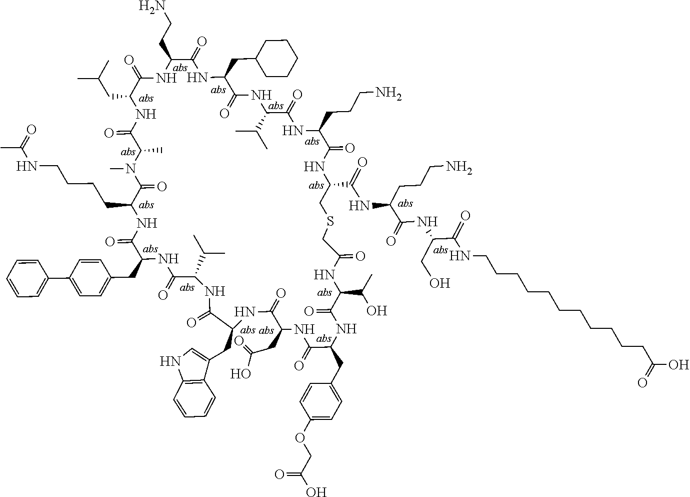 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2406 was prepared on a 50 μmol scale. The yield of the product was 28.3 mg, and its estimated purity by LCMS analysis was 98.8%. Analysis condition B: Retention time=1.73 min; ESI-MS(+) m/z [M+2H]²⁺: 781.2.

### Preparation of Example 2407

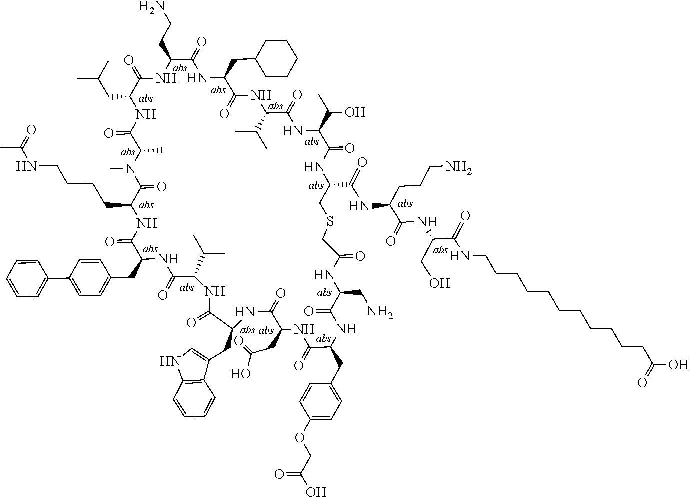 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2407 was prepared on a 50 μmol scale. The yield of the product was 25.6 mg, and its estimated purity by LCMS analysis was 97.1%. Analysis condition B: Retention time=1.68 min; ESI-MS(+) m/z [M+2H]²⁺: 1157.2.

### Preparation of Example 2408

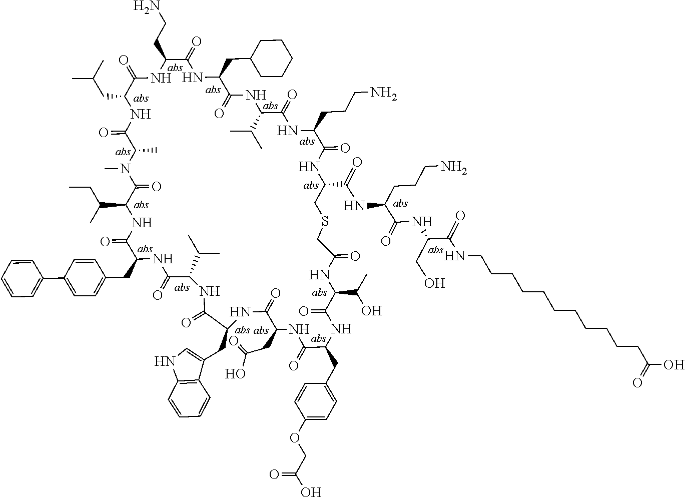 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2408 was prepared on a 50 μmol scale. The yield of the product was 6.5 mg, and its estimated purity by LCMS analysis was 97.7%. Analysis condition B: Retention time=1.77 min; ESI-MS(+) m/z [M+3H]³⁺: 762.

### Preparation of Example 2409

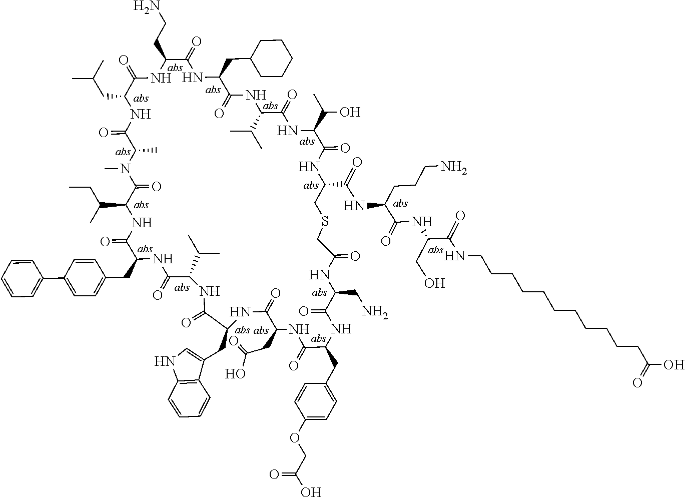 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2409 was prepared on a 50 μmol scale. The yield of the product was 11.6 mg, and its estimated purity by LCMS analysis was 96.8%. Analysis condition A: Retention time=1.7 min; ESI-MS(+) m/z [M+2H]²⁺: 1128.3.

### Preparation of Example 2410

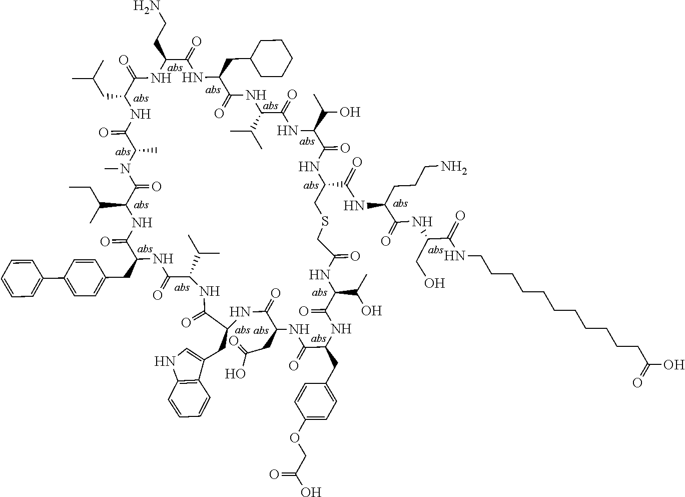 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2410 was prepared on a 50 μmol scale. The yield of the product was 13.4 mg, and its estimated purity by LCMS analysis was 98.6%. Analysis condition B: Retention time=1.84 min; ESI-MS(+) m/z [M+2H]²⁺: 1136.

### Preparation of Example 2411

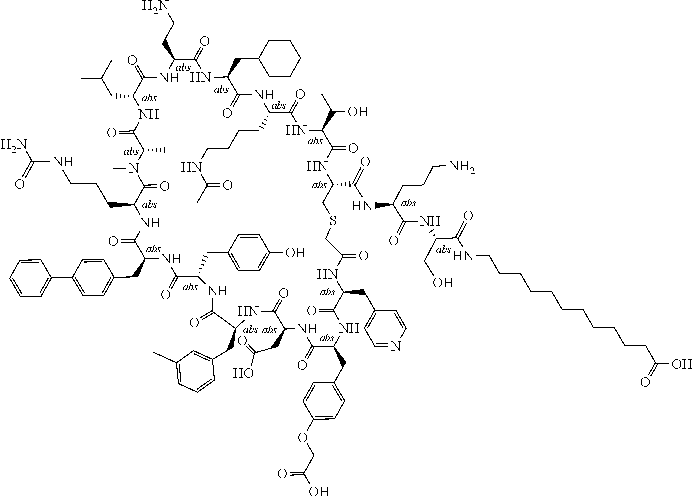 [SMILES: CC(=O)NCCCC[C@@H]1NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC1=O]

Example 2411 was prepared on a 50 μmol scale. The yield of the product was 4.3 mg, and its estimated purity by LCMS analysis was 96.9%. Analysis condition A: Retention time=1.63 min; ESI-MS(+) m/z [M+2H]²⁺: 1236.3.

### Preparation of Example 2412

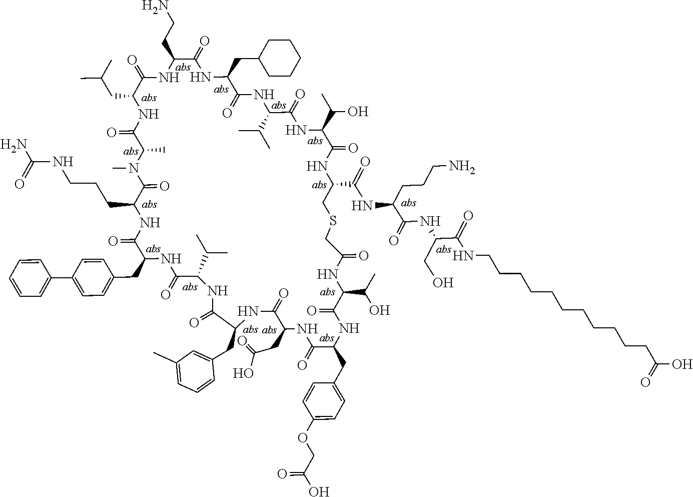 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2412 was prepared on a 50 μmol scale. The yield of the product was 4.7 mg, and its estimated purity by LCMS analysis was 99.3%. Analysis condition A: Retention time=1.63 min; ESI-MS(+) m/z [M+2H]²⁺: 1145.2.

### Preparation of Example 2413

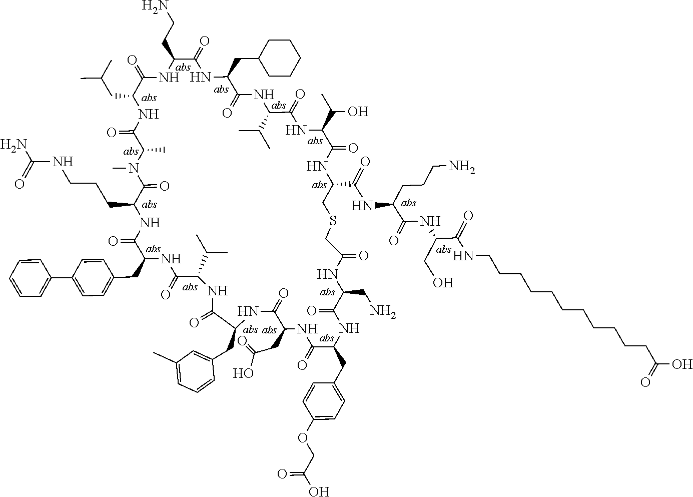 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(N)=O)NC(=O)[C@H](Cc3ccc(-c4ccccc4)cc3)NC(=O)[C@H](C(C)C)NC2=O)c1]

Example 2413 was prepared on a 50 μmol scale. The yield of the product was 11.2 mg, and its estimated purity by LCMS analysis was 95.3%. Analysis condition B: Retention time=1.75 min; ESI-MS(+) m/z [M+2H]²⁺: 1138.1.

### Preparation of Example 2414

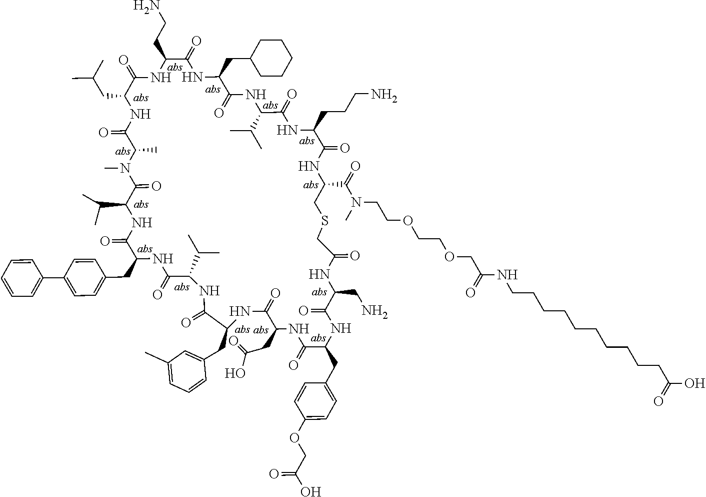 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N(C)CCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](C(C)C)NC(=O)[C@H](Cc3ccc(-c4ccccc4)cc3)NC(=O)[C@H](C(C)C)NC2=O)c1]

Example 2414 was prepared on a 50 μmol scale. The yield of the product was 8.9 mg, and its estimated purity by LCMS analysis was 96.5%. Analysis condition A: Retention time=1.65 min; ESI-MS(+) m/z [M+3H]³⁺: 725.4.

### Preparation of Example 2415

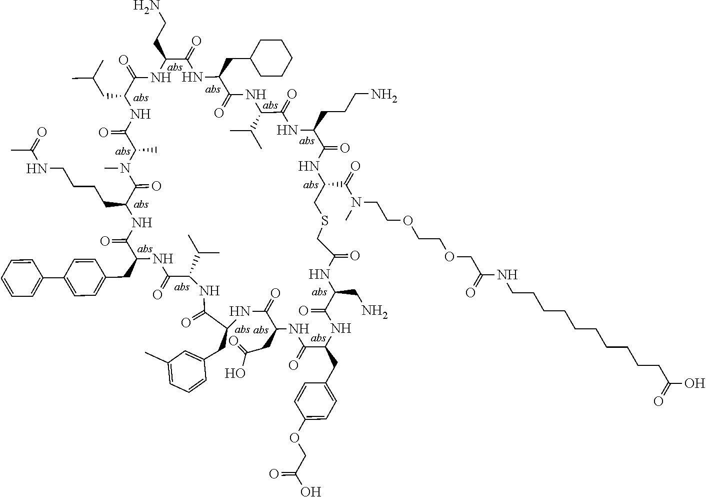 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N(C)CCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2415 was prepared on a 50 μmol scale. The yield of the product was 11.9 mg, and its estimated purity by LCMS analysis was 96.2%. Analysis condition A: Retention time=1.62 min; ESI-MS(+) m/z [M+2H]²⁺: 1123.1.

### Preparation of Example 2416

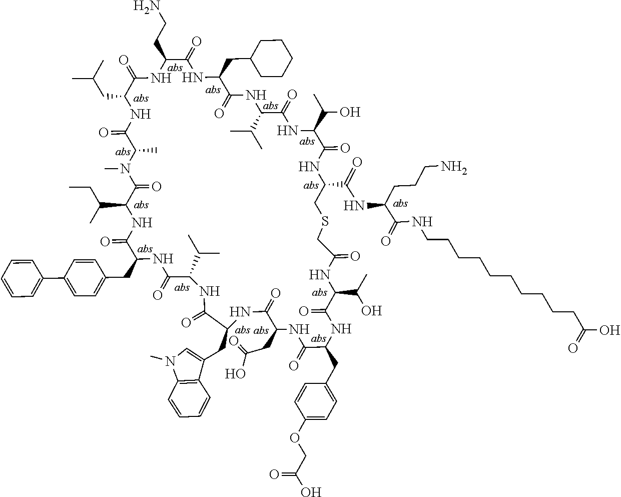 [SMILES: CCC(C)[C@@H]1NC(=O)[C@H](Cc2ccc(-c3ccccc3)cc2)NC(=O)[C@H](C(C)C)NC(=O)[C@H](Cc2cn(C)c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2416 was prepared on a 50 μmol scale. The yield of the product was 15.6 mg, and its estimated purity by LCMS analysis was 95.5%. Analysis condition B: Retention time=1.87 min; ESI-MS(+) m/z [M+2H]²⁺: 1093.1.

### Preparation of Example 2417

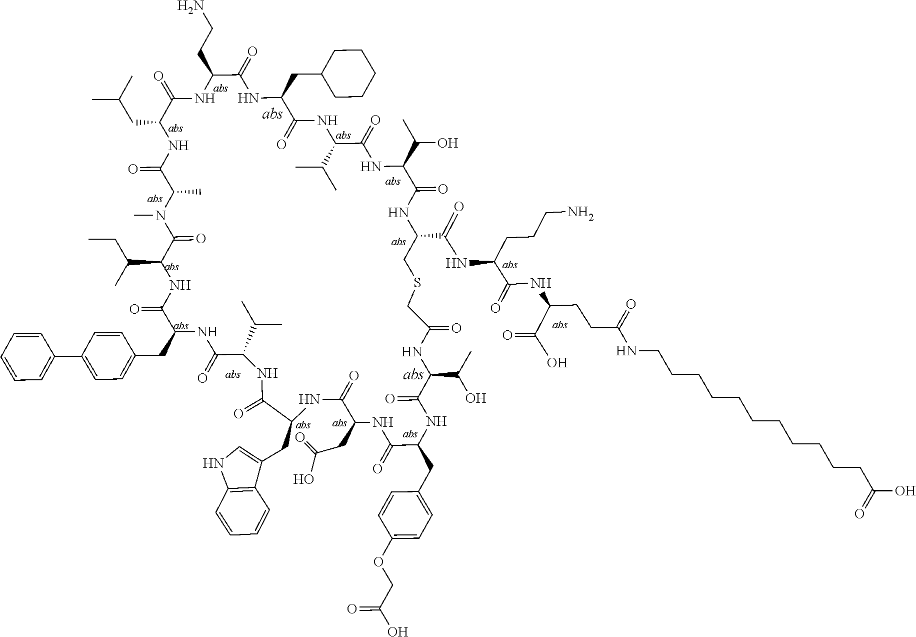 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CCC(=O)NCCCCCCCCCCCC(=O)O)C(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2417 was prepared on a 50 μmol scale. The yield of the product was 7.4 mg, and its estimated purity by LCMS analysis was 100%. Analysis condition A: Retention time=1.63 min; ESI-MS(+) m/z [M+2H]²⁺: 1157.1.

### Preparation of Example 2418

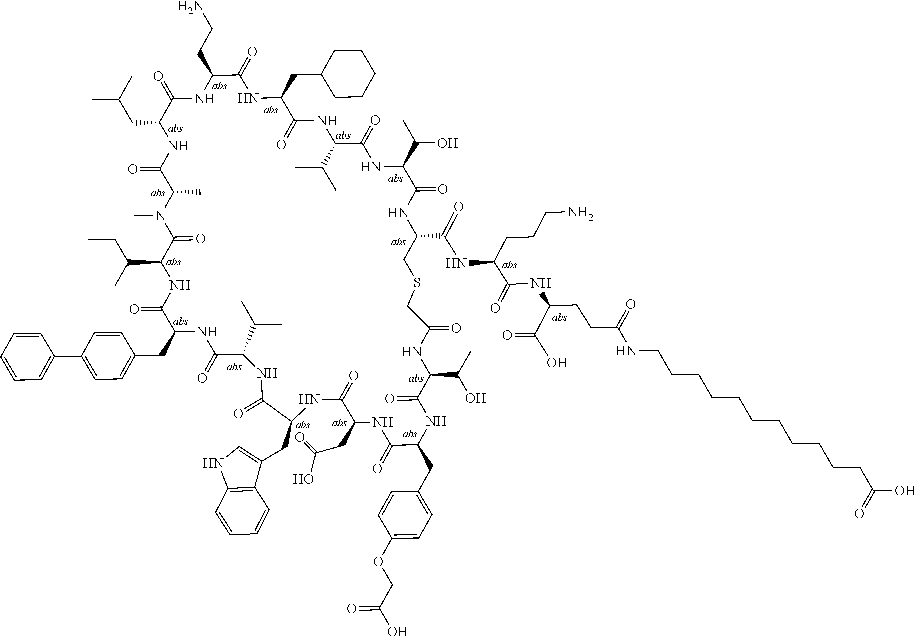 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)N[C@@H](CCC(=O)NCCCCCCCCCCCC(=O)O)C(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2418 was prepared on a 50 μmol scale. The yield of the product was 25.8 mg, and its estimated purity by LCMS analysis was 96%. Analysis condition B: Retention time=1.9 min; ESI-MS(+) m/z [M+2H]²⁺: 1157.

### Preparation of Example 2419

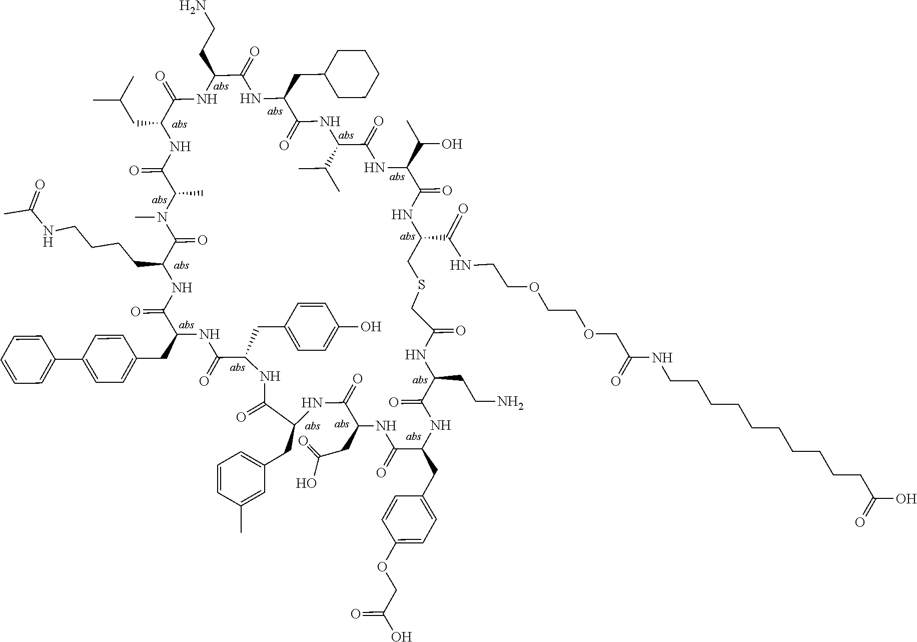 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2419 was prepared on a 50 μmol scale. The yield of the product was 11.9 mg, and its estimated purity by LCMS analysis was 95%. Analysis condition B: Retention time=1.89 min; ESI-MS(+) m/z [M+3H]³⁺: 765.6.

### Preparation of Example 2420

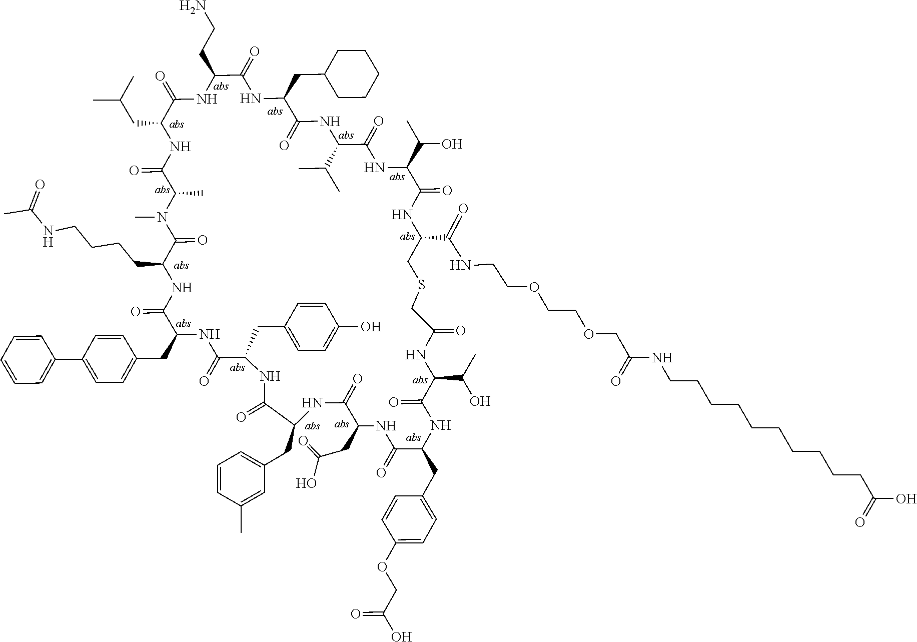 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2420 was prepared on a 50 μmol scale. The yield of the product was 40.1 mg, and its estimated purity by LCMS analysis was 100%. Analysis condition A: Retention time=1.72 min; ESI-MS(+) m/z [M+2H]²⁺: 1149.2.

### Preparation of Example 2421

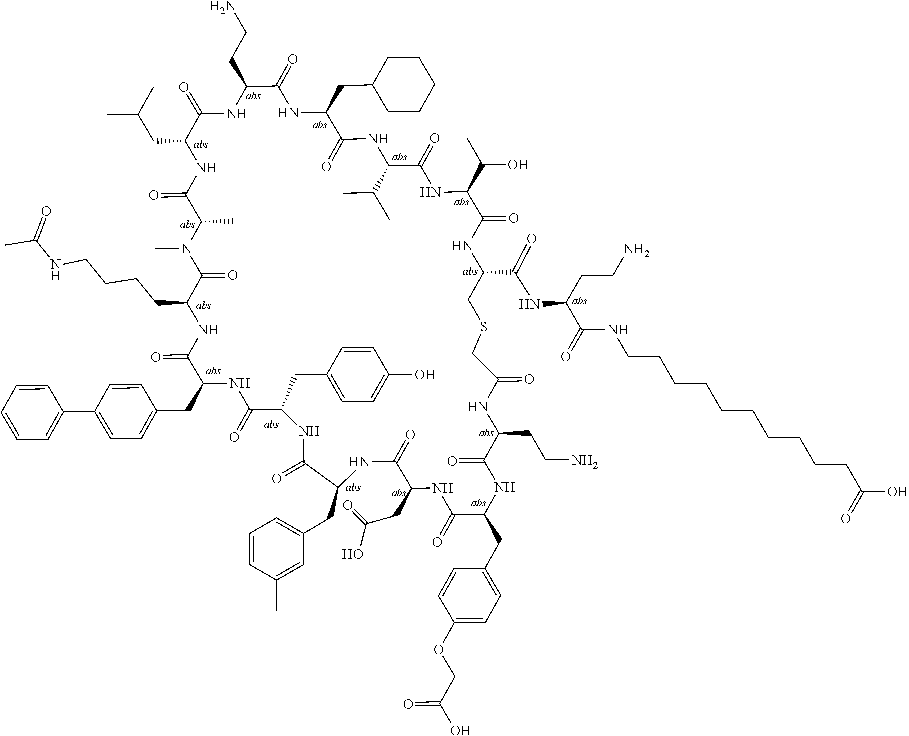 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2421 was prepared on a 50 μmol scale. The yield of the product was 75 mg, and its estimated purity by LCMS analysis was 90.4%. Analysis condition B: Retention time=1.71 min; ESI-MS(+) m/z [M+2H]²⁺: 1126.2.

### Preparation of Example 2422

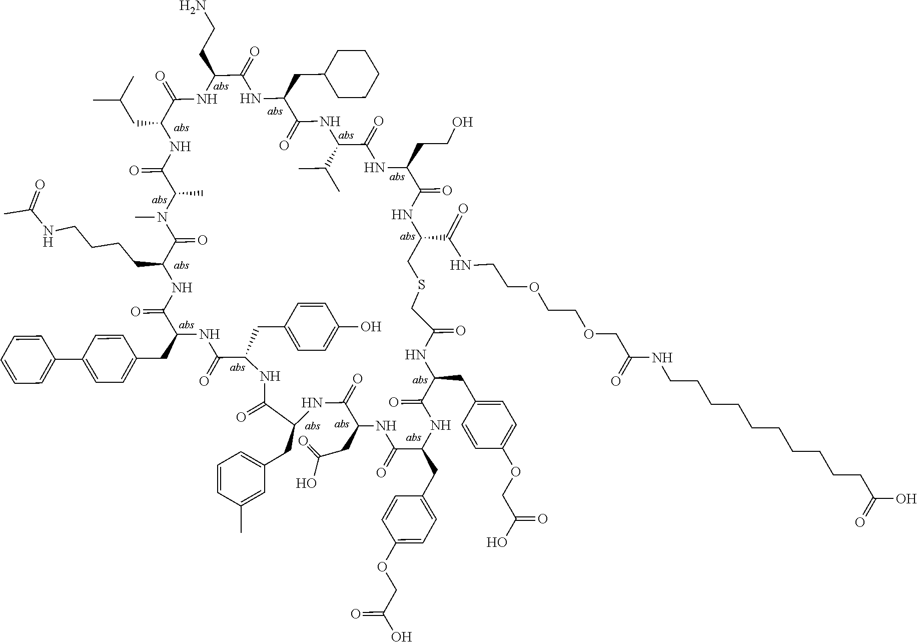 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2422 was prepared on a 50 μmol scale. The yield of the product was 19.9 mg, and its estimated purity by LCMS analysis was 95.3%. Analysis condition B: Retention time=1.92 min; ESI-MS(+) m/z [M+2H]²⁺: 1208.9.

### Preparation of Example 2423

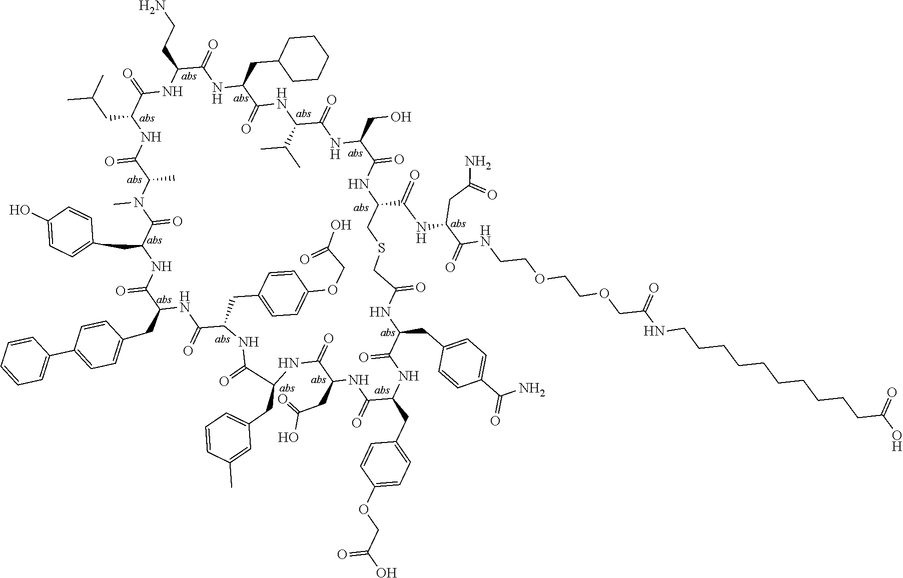 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2423 was prepared on a 50 μmol scale. The yield of the product was 60.9 mg, and its estimated purity by LCMS analysis was 95.8%. Analysis condition B: Retention time=1.91 min; ESI-MS(+) m/z [M+2H]²⁺: 1269.1.

### Preparation of Example 2424

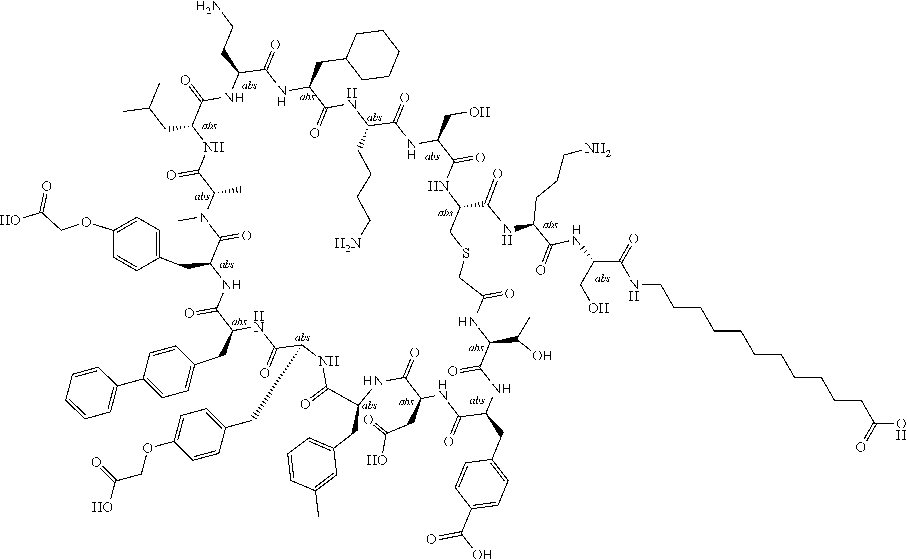 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](CCCCN)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2424 was prepared on a 50 μmol scale. The yield of the product was 10.4 mg, and its estimated purity by LCMS analysis was 100%. Analysis condition A: Retention time=1.54 min; ESI-MS(+) m/z [M+2H]²⁺: 1231.

### Preparation of Example 2425

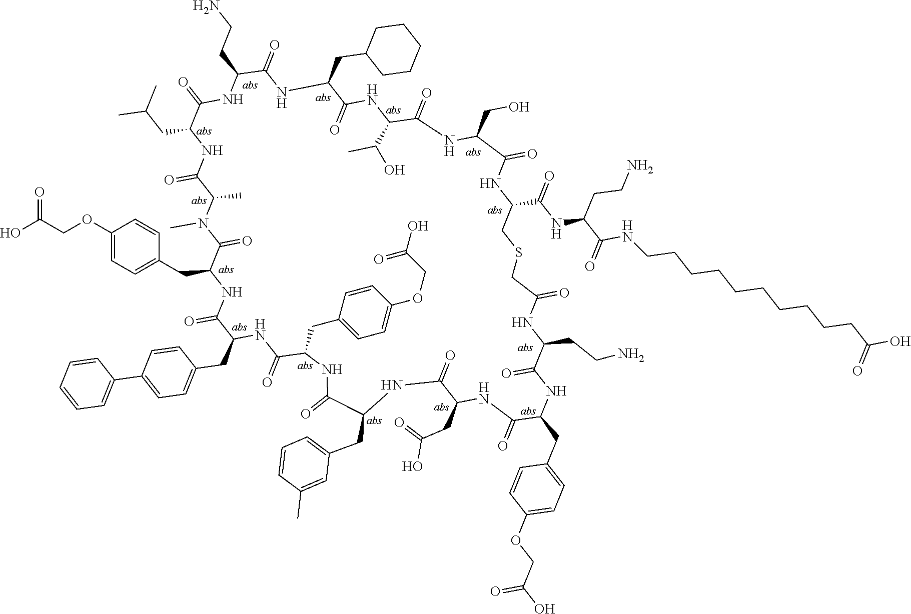 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2425 was prepared on a 50 μmol scale. The yield of the product was 6.6 mg, and its estimated purity by LCMS analysis was 97%. Analysis condition B: Retention time=1.82 min; ESI-MS(+) m/z [M+2H]²⁺: 1174.2.

### Preparation of Example 2426

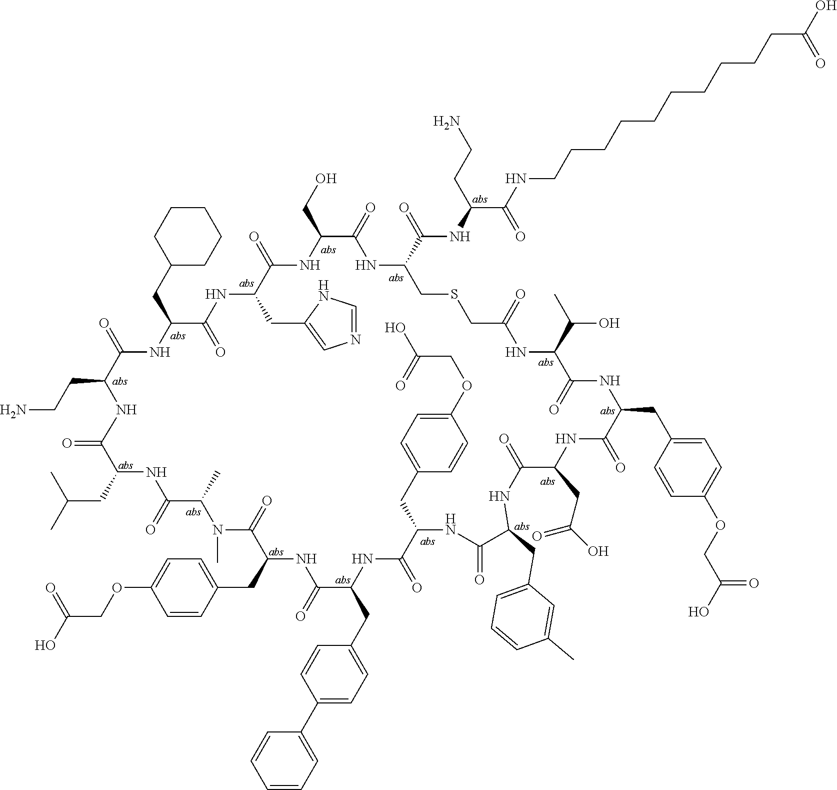 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2426 was prepared on a 50 μmol scale. The yield of the product was 17.2 mg, and its estimated purity by LCMS analysis was 95.7%. Analysis condition B: Retention time=1.74 min; ESI-MS(+) m/z [M+2H]²⁺: 1193.2.

### Preparation of Example 2427

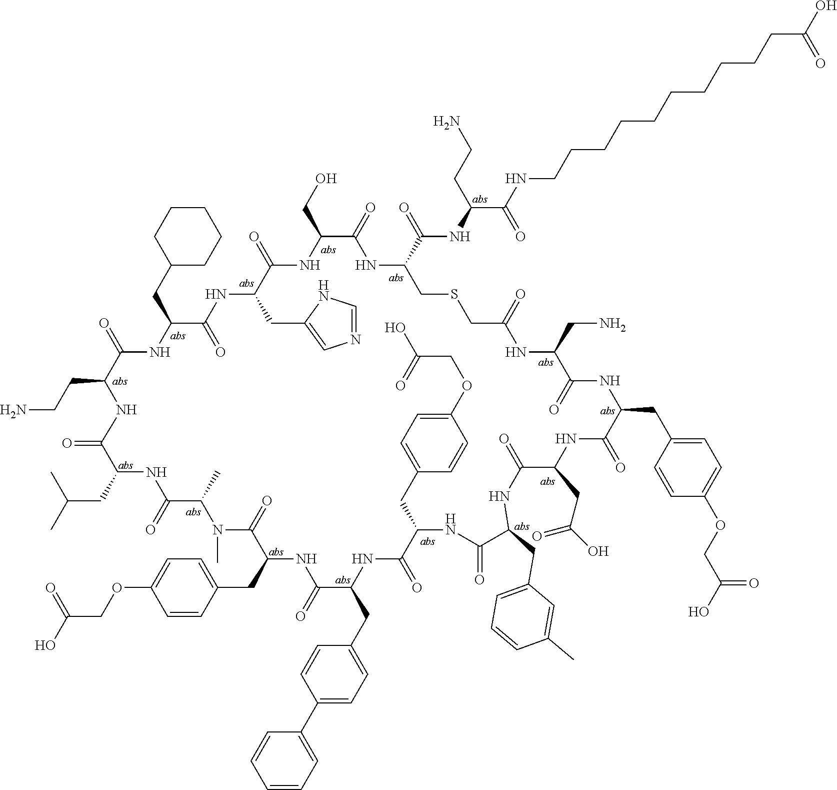 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2427 was prepared on a 50 μmol scale. The yield of the product was 20.3 mg, and its estimated purity by LCMS analysis was 93.4%. Analysis condition B: Retention time=1.67 min; ESI-MS(+) m/z [M+2H]²⁺: 1185.

### Preparation of Example 2428

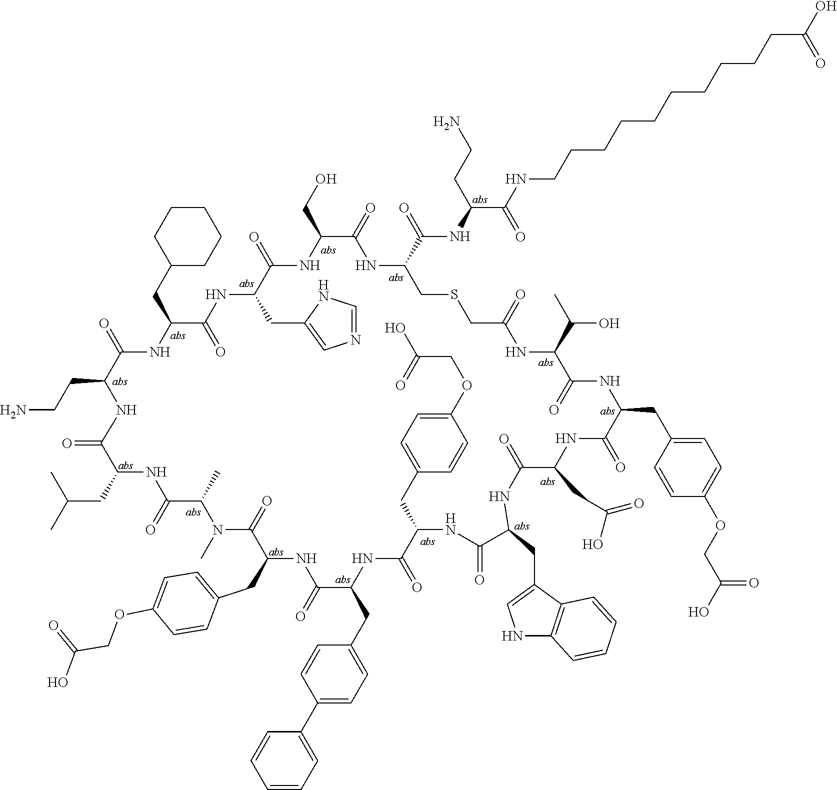 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCN)NC1=O]

Example 2428 was prepared on a 50 μmol scale. The yield of the product was 9.4 mg, and its estimated purity by LCMS analysis was 100%. Analysis condition A: Retention time=1.61 min; ESI-MS(+) m/z [M+2H]²⁺: 1205.3.

### Preparation of Example 2429

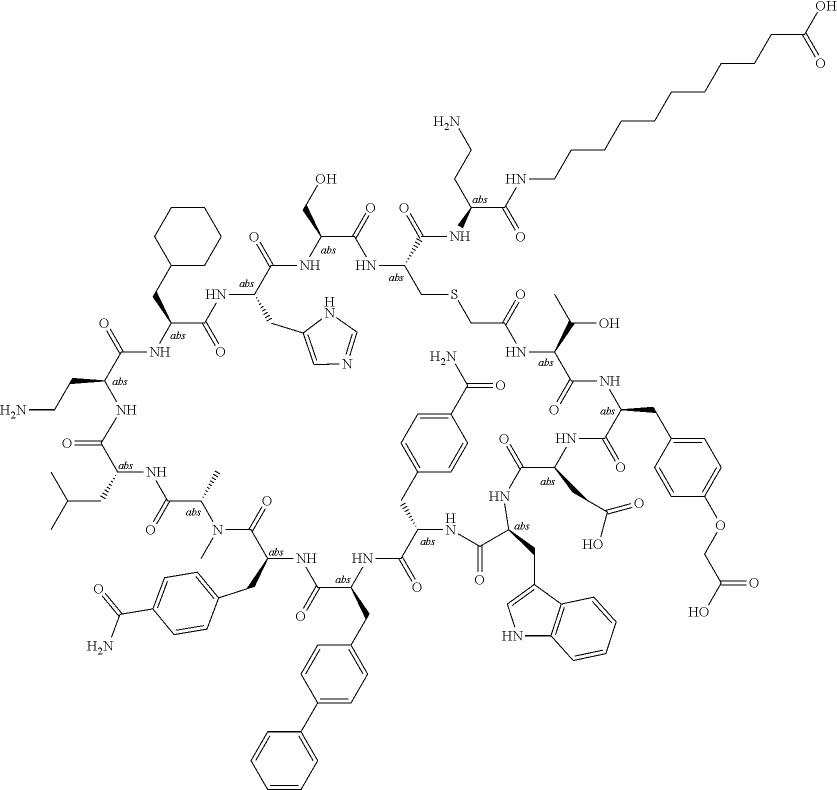 [SMILES: CC(C)C[C@H]1NC(=O)[C@H](C)N(C)C(=O)[C@H](Cc2ccc(C(N)=O)cc2)NC(=O)[C@H](Cc2ccc(-c3ccccc3)cc2)NC(=O)[C@H](Cc2ccc(C(N)=O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCN)NC1=O]

Example 2429 was prepared on a 50 μmol scale. The yield of the product was 11.6 mg, and its estimated purity by LCMS analysis was 95.5%. Analysis condition B: Retention time=1.74 min; ESI-MS(+) m/z [M+3H]³⁺: 783.1.

### Preparation of Example 2430

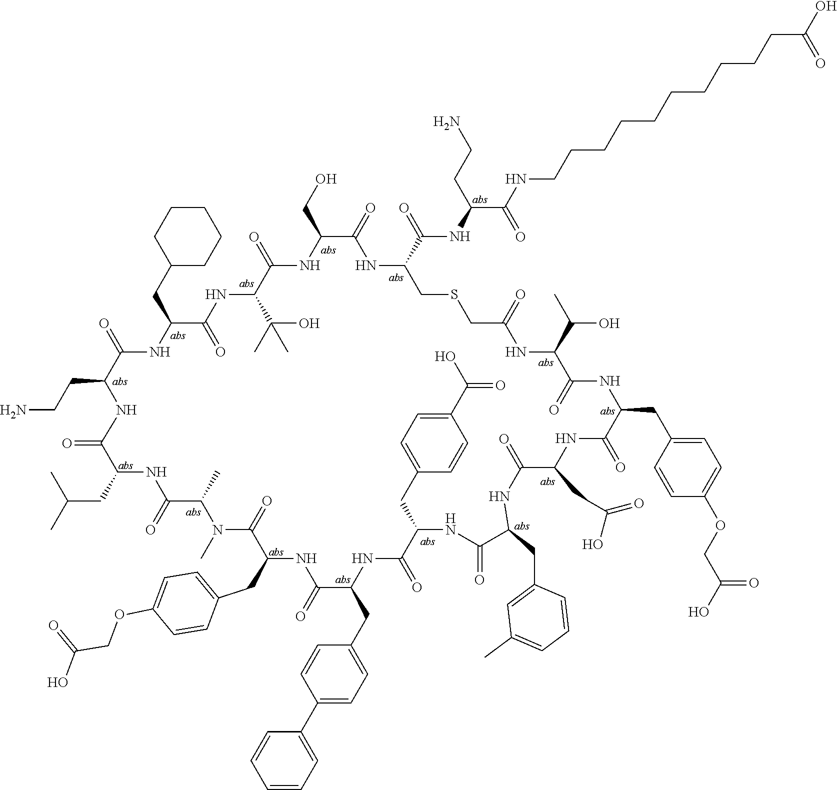 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2430 was prepared on a 50 μmol scale. The yield of the product was 16.7 mg, and its estimated purity by LCMS analysis was 95.2%. Analysis condition A: Retention time=1.52 min; ESI-MS(+) m/z [M+2H]²⁺: 1167.

### Preparation of Example 2431

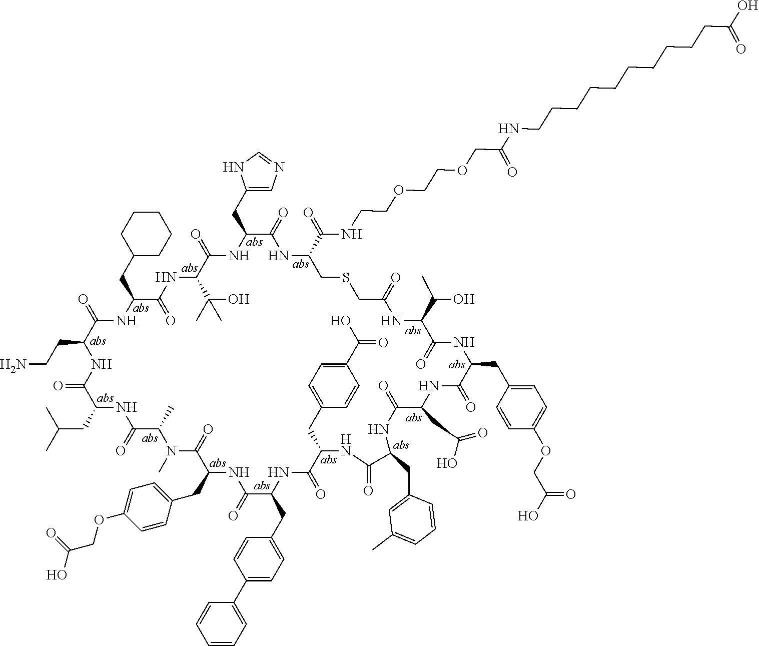 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2431 was prepared on a 50 μmol scale. The yield of the product was 11 mg, and its estimated purity by LCMS analysis was 99.2%. Analysis condition A: Retention time=1.43 min; ESI-MS(+) m/z [M+2H]²⁺: 1214.8.

### Preparation of Example 2432

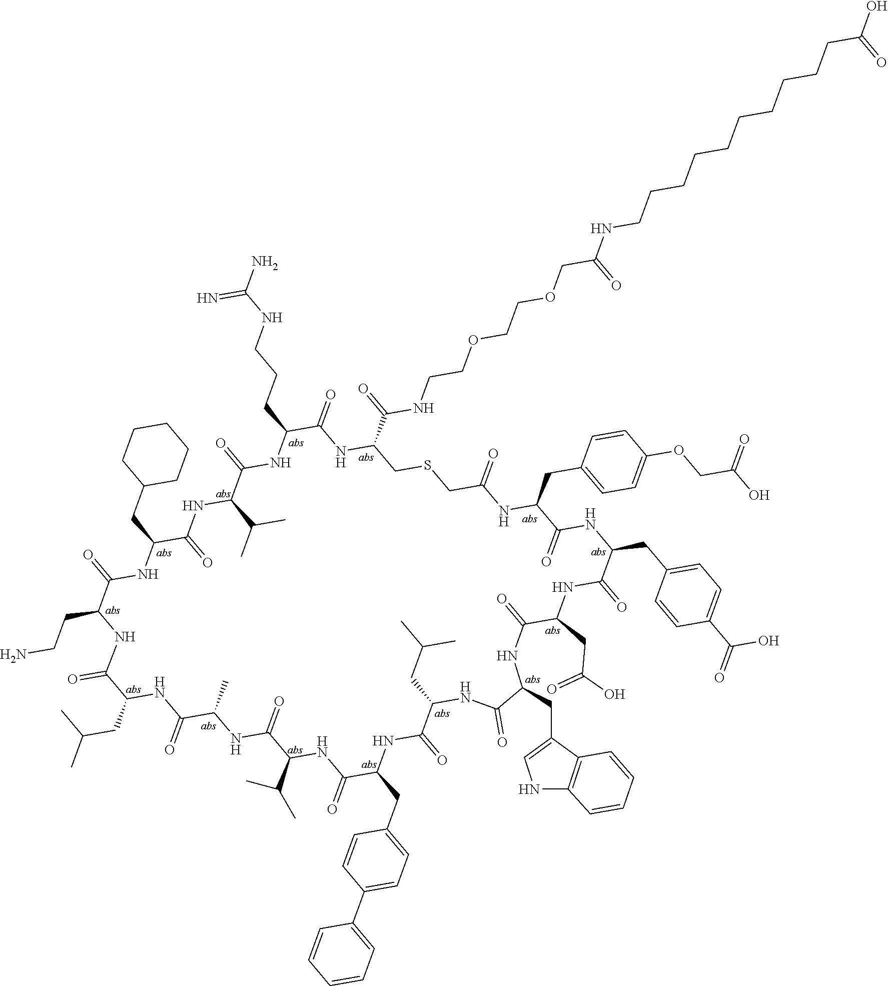 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(=N)N)NC(=O)[C@@H](C(C)C)NC(=O)[C@H](CC2CCCCC2)NC(=O)[C@H](CCN)NC(=O)[C@@H](CC(C)C)NC(=O)[C@H](C)NC(=O)[C@H](C(C)C)NC(=O)[C@H](Cc2ccc(-c3ccccc3)cc2)NC1=O]

Example 2432 was prepared on a 50 μmol scale. The yield of the product was 15.3 mg, and its estimated purity by LCMS analysis was 100%. Analysis condition A: Retention time=1.89 min; ESI-MS(+) m/z [M+2H]²⁺: 1173.

### Preparation of Example 2433

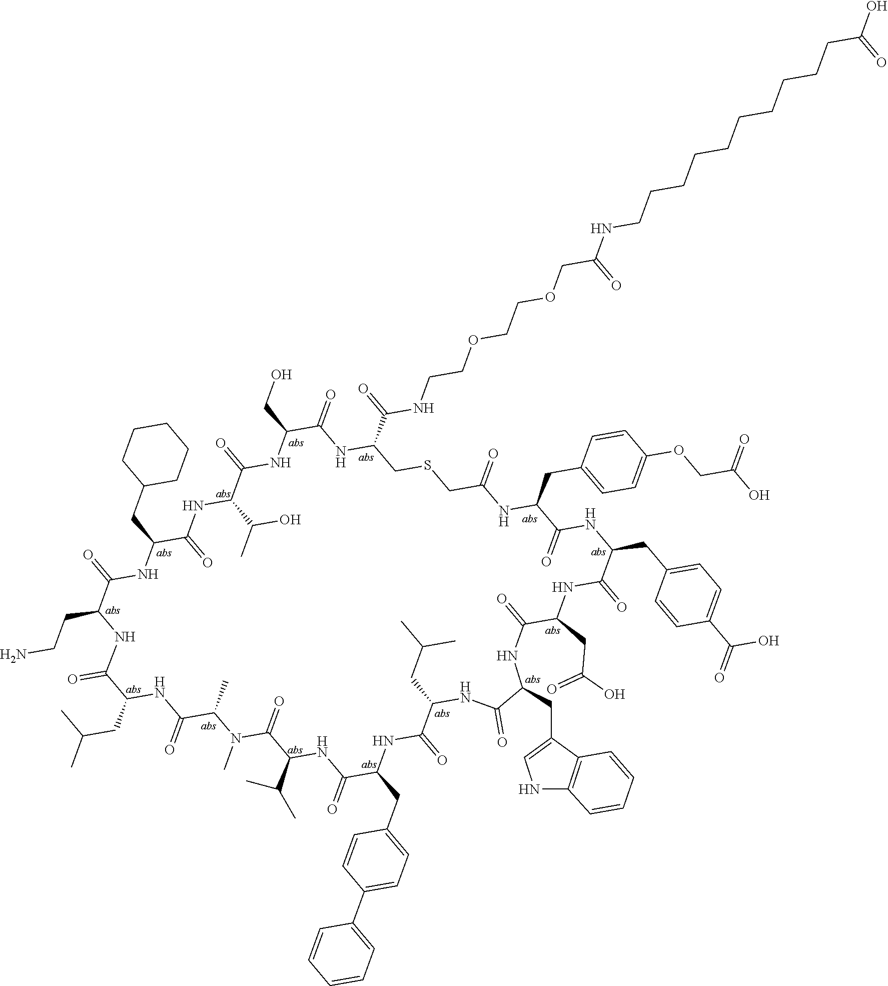 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2433 was prepared on a 50 μmol scale. The yield of the product was 12.8 mg, and its estimated purity by LCMS analysis was 95.7%. Analysis condition A: Retention time=1.77 min; ESI-MS(+) m/z [M+2H]²⁺: 1140.2.

### Preparation of Example 2434

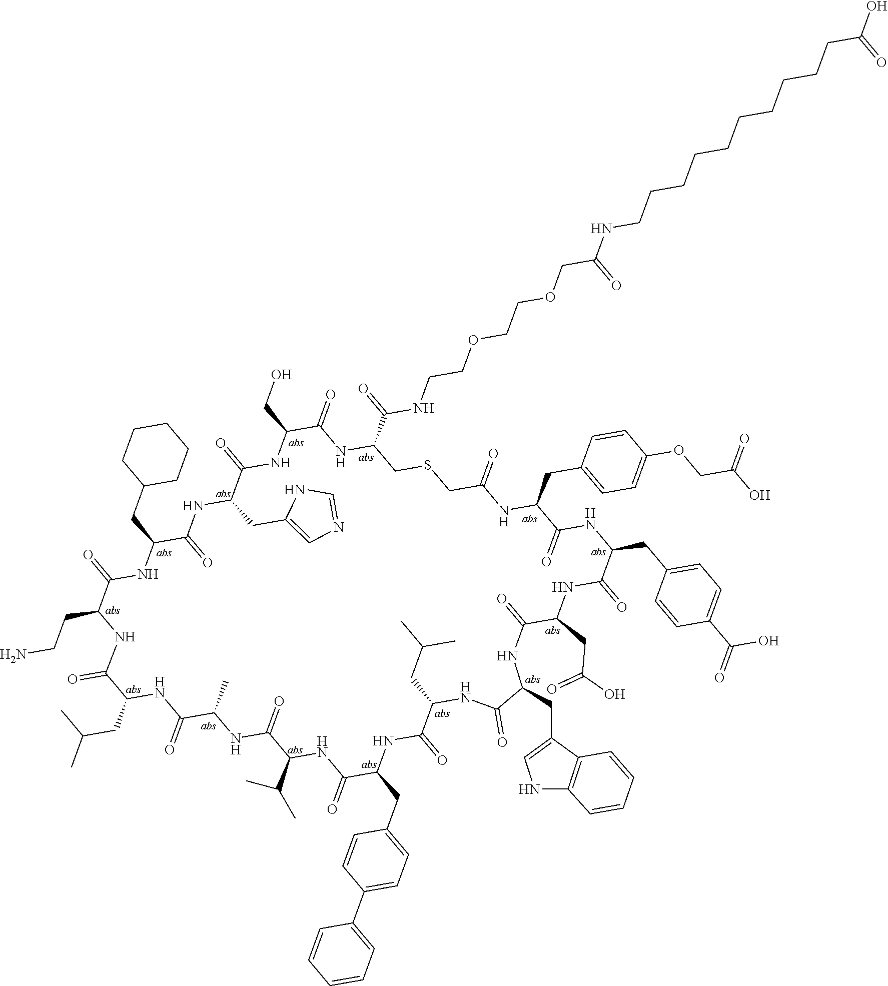 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCN)NC(=O)[C@@H](CC(C)C)NC(=O)[C@H](C)NC(=O)[C@H](C(C)C)NC(=O)[C@H](Cc2ccc(-c3ccccc3)cc2)NC1=O]

Example 2434 was prepared on a 50 μmol scale. The yield of the product was 5.4 mg, and its estimated purity by LCMS analysis was 97.7%. Analysis condition B: Retention time=1.98 min; ESI-MS(+) m/z [M+2H]²⁺: 1158.1.

### Preparation of Example 2435

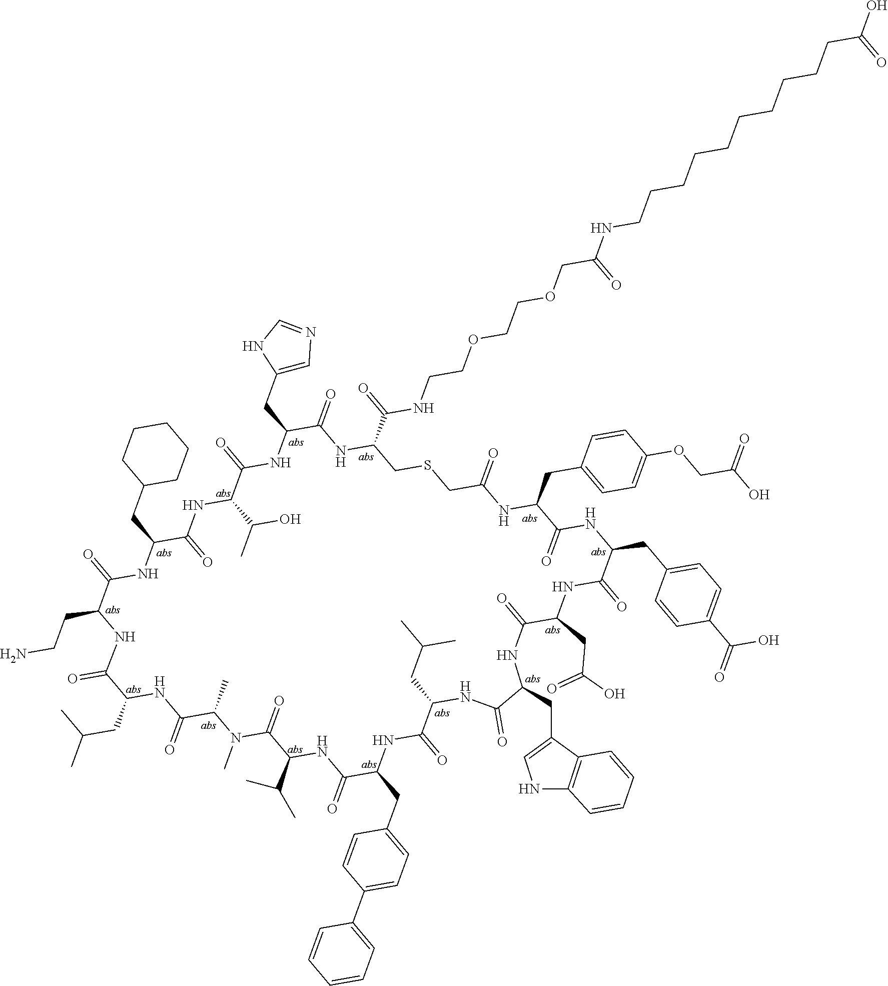 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2435 was prepared on a 50 μmol scale. The yield of the product was 36.1 mg, and its estimated purity by LCMS analysis was 96%. Analysis condition A: Retention time=1.74 min; ESI-MS(+) m/z [M+2H]²⁺: 1165.3.

### Preparation of Example 2436

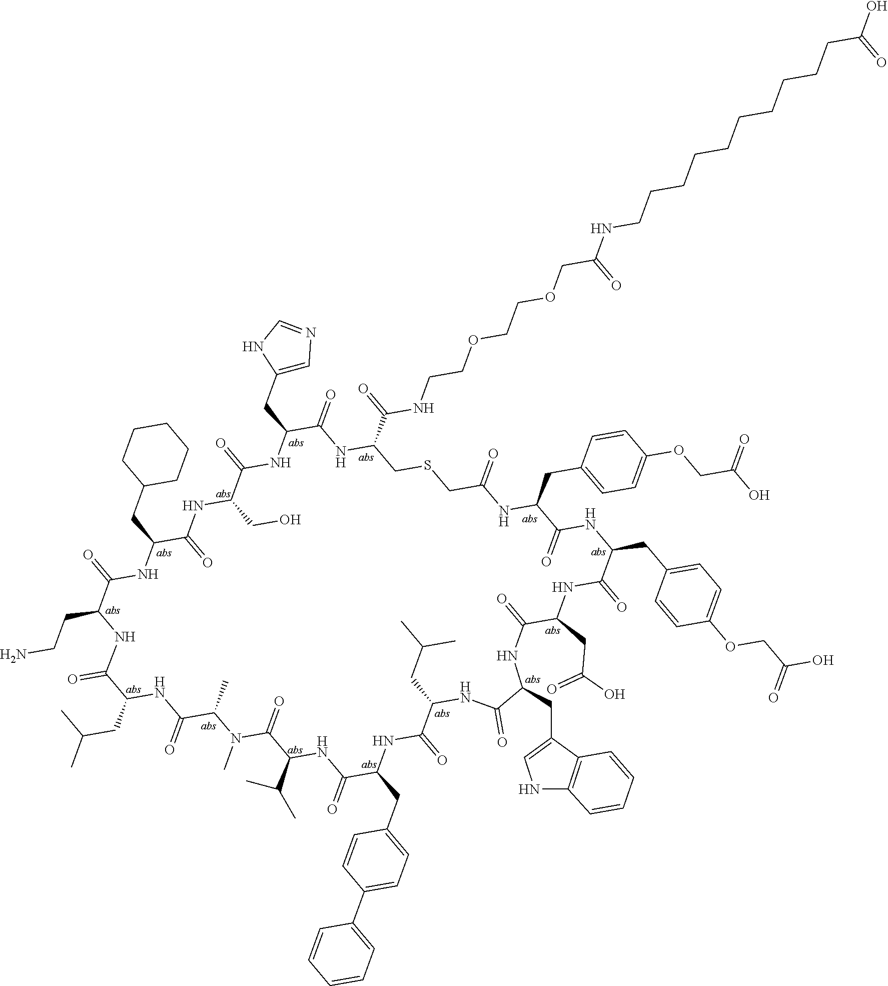 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2cnc[nH]2)NC(=O)[C@H](CO)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2436 was prepared on a 50 μmol scale. The yield of the product was 17.8 mg, and its estimated purity by LCMS analysis was 95.8%. Analysis condition A: Retention time=1.71 min; ESI-MS(+) m/z [M+2H]²⁺: 1173.1.

### Preparation of Example 2437

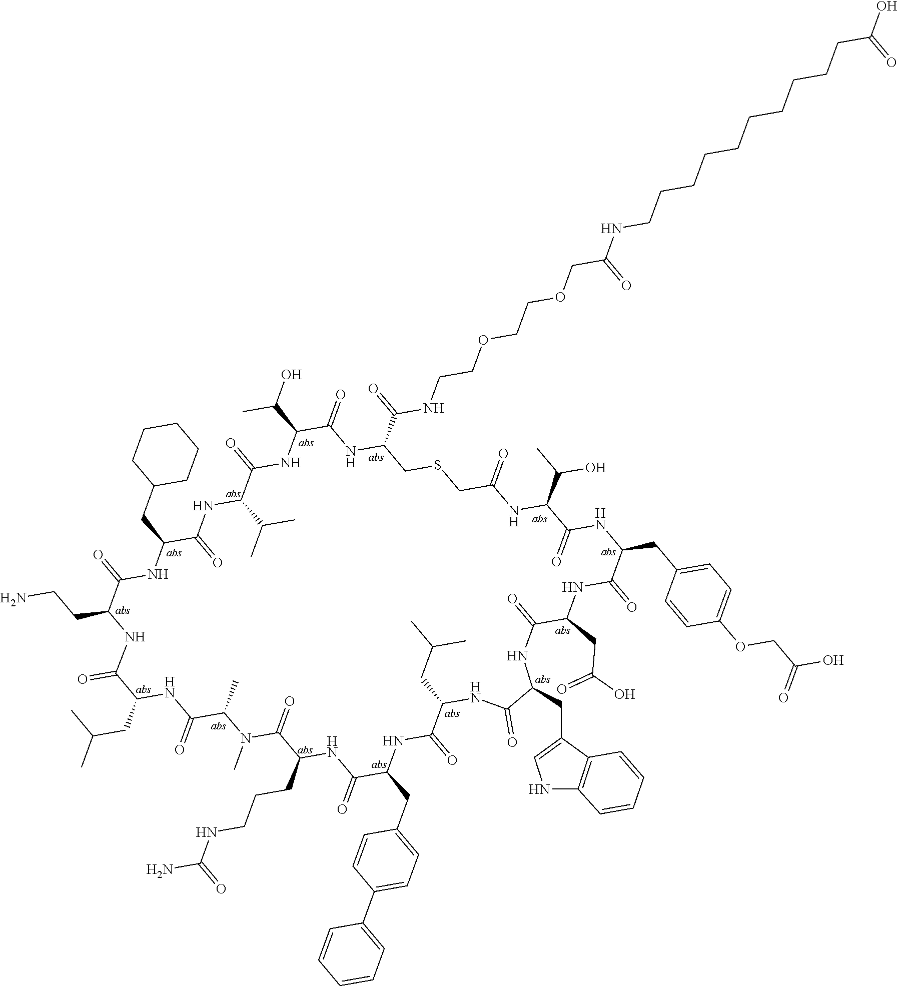 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC1=O]

Example 2437 was prepared on a 50 μmol scale. The yield of the product was 25.9 mg, and its estimated purity by LCMS analysis was 90.2%. Analysis condition A: Retention time=1.84 min; ESI-MS(+) m/z [M+2H]²⁺: 1129.8.

### Preparation of Example 2438

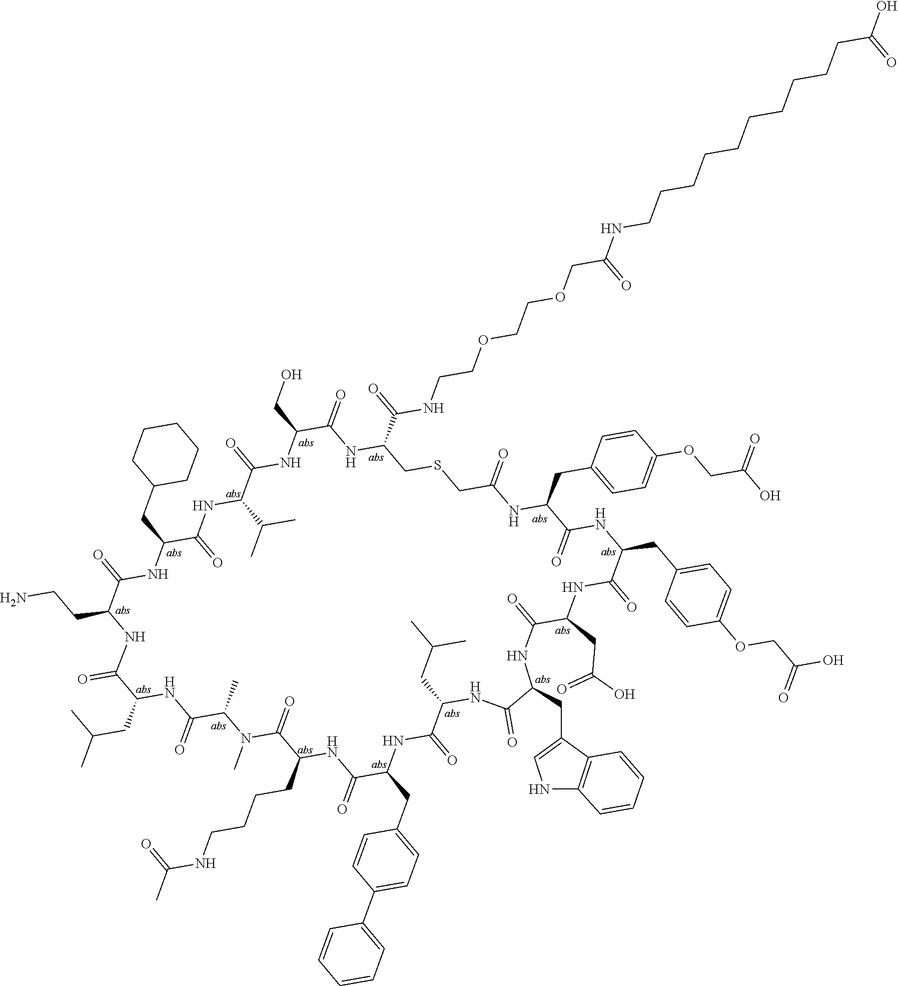 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2438 was prepared on a 50 μmol scale. The yield of the product was 29.2 mg, and its estimated purity by LCMS analysis was 96.3%. Analysis condition A: Retention time=1.73 min; ESI-MS(+) m/z [M+2H]²⁺: 1189.9.

### Preparation of Example 2439

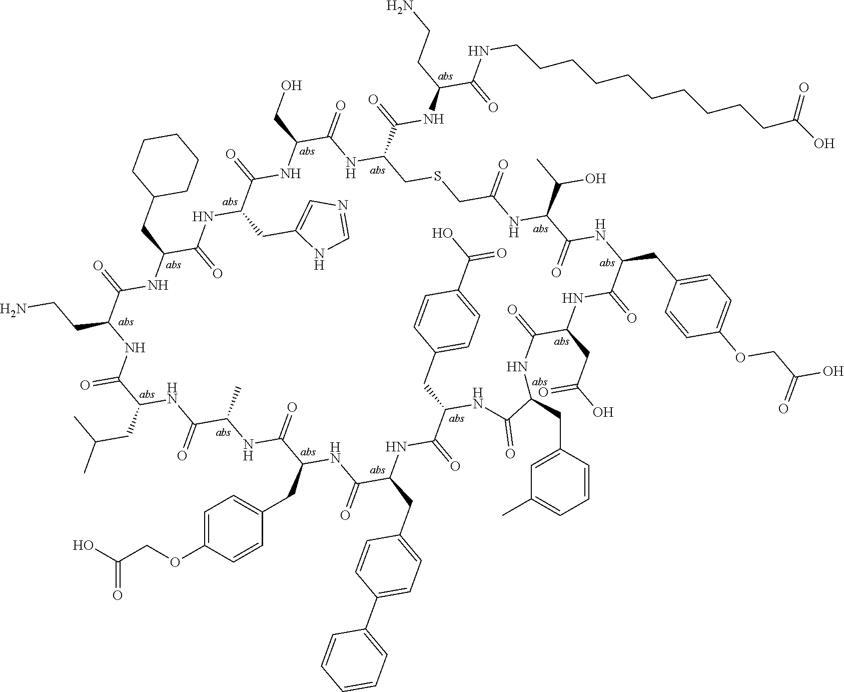 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](C)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2439 was prepared on a 50 μmol scale. The yield of the product was 14.6 mg, and its estimated purity by LCMS analysis was 94.9%. Analysis condition B: Retention time=1.73 min; ESI-MS(+) m/z [M+2H]²⁺: 1178.3.

### Preparation of Example 2440

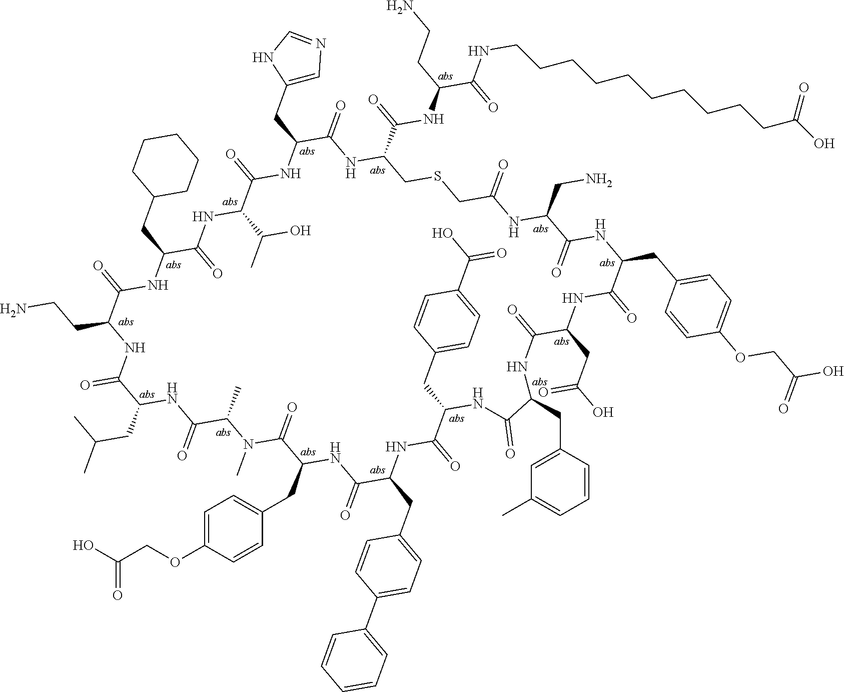 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2440 was prepared on a 50 μmol scale. The yield of the product was 16.9 mg, and its estimated purity by LCMS analysis was 98.3%. Analysis condition B: Retention time=1.73 min; ESI-MS(+) m/z [M+2H]²⁺: 1185.3.

### Preparation of Example 2441

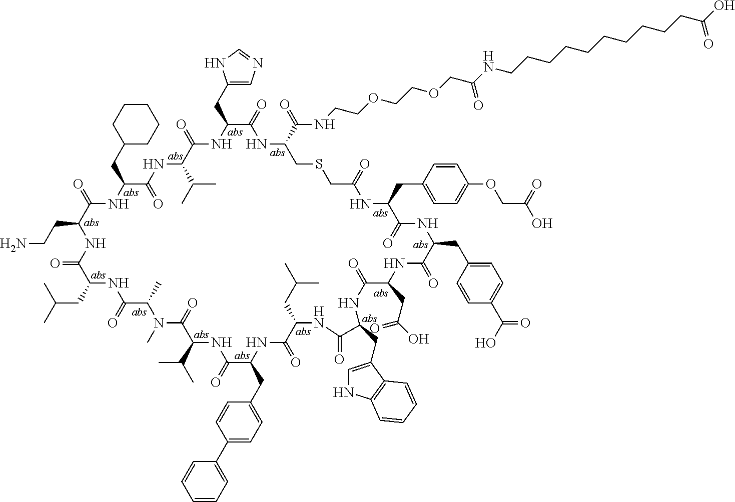 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2cnc[nH]2)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2441 was prepared on a 50 μmol scale. The yield of the product was 29.9 mg, and its estimated purity by LCMS analysis was 97.4%. Analysis condition B: Retention time=1.91 min; ESI-MS(+) m/z [M+2H]²⁺: 1164.1.

### Preparation of Example 2442

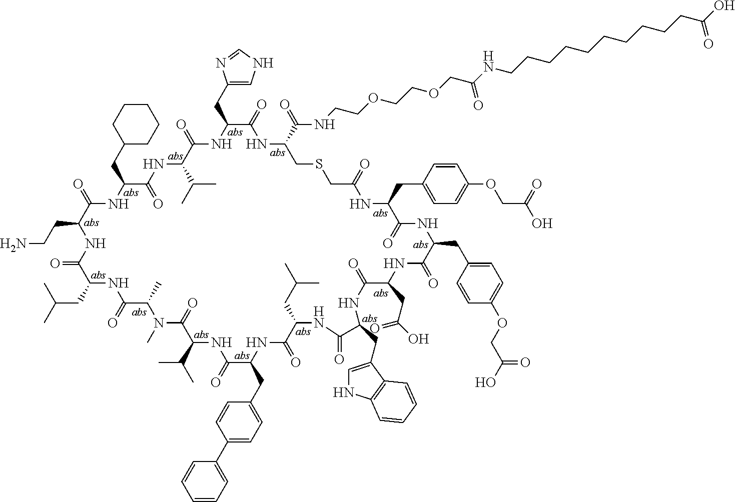 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2c[nH]cn2)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2442 was prepared on a 50 μmol scale. The yield of the product was 28.4 mg, and its estimated purity by LCMS analysis was 95.3%. Analysis condition B: Retention time=1.96 min; ESI-MS(+) m/z [M+2H]²⁺: 1179.1

### Preparation of Example 2443

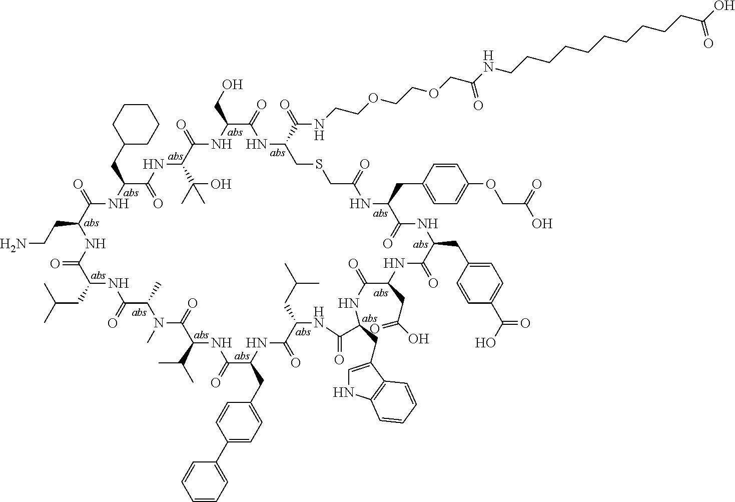 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2443 was prepared on a 50 μmol scale. The yield of the product was 44.8 mg, and its estimated purity by LCMS analysis was 96.5%. Analysis condition A: Retention time=1.9 min; ESI-MS(+) m/z [M+2H]²⁺: 1147.

### Preparation of Example 2444

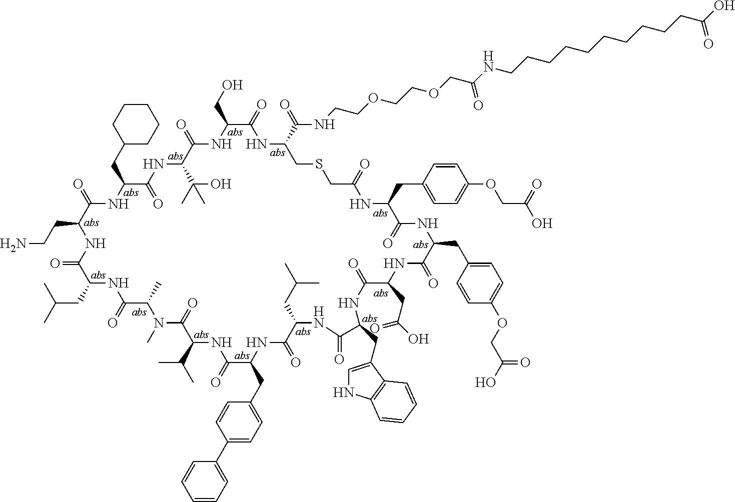 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2444 was prepared on a 50 μmol scale. The yield of the product was 59.8 mg, and its estimated purity by LCMS analysis was 95.8%. Analysis condition A: Retention time=1.7 min; ESI-MS(+) m/z [M+2H]²⁺: 1161.9.

### Preparation of Example 2445

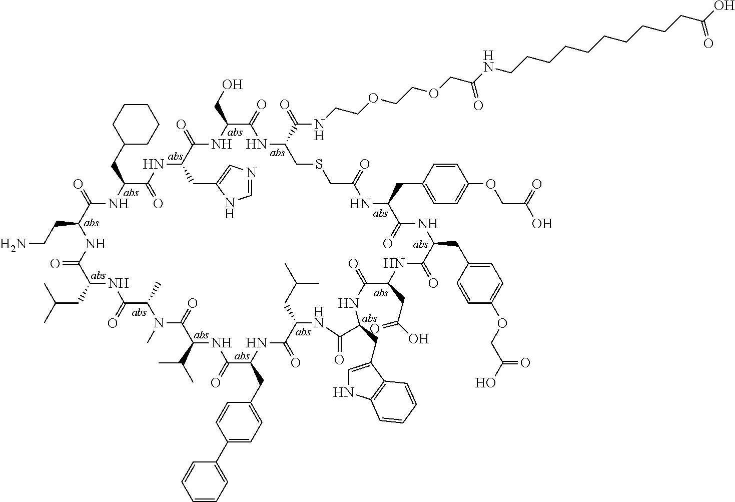 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2445 was prepared on a 50 μmol scale. The yield of the product was 35.4 mg, and its estimated purity by LCMS analysis was 97.8%. Analysis condition B: Retention time=2.02 min; ESI-MS(+) m/z [M+2H]²⁺: 1173.1.

### Preparation of Example 2446

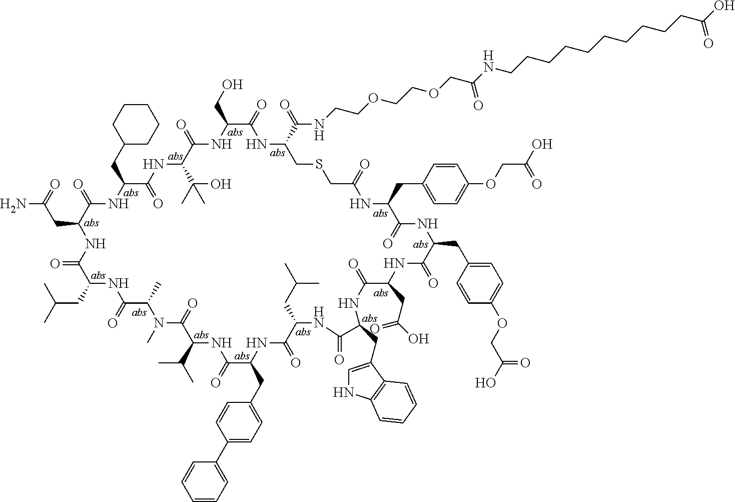 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CC(N)=O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2446 was prepared on a 50 μmol scale. The yield of the product was 5 mg, and its estimated purity by LCMS analysis was 97.5%. Analysis condition B: Retention time=2.31 min; ESI-MS(+) m/z [M+2H]²⁺: 1169.1.

### Preparation of Example 2447

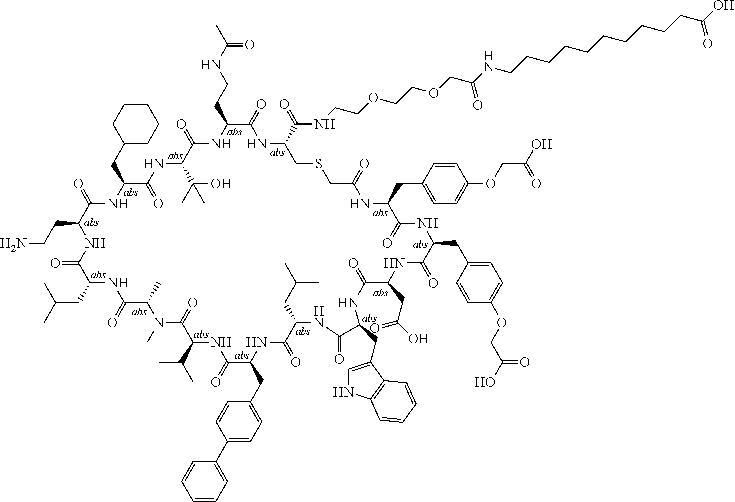 [SMILES: CC(=O)NCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC1=O]

Example 2447 was prepared on a 50 μmol scale. The yield of the product was 21.1 mg, and its estimated purity by LCMS analysis was 99%. Analysis condition B: Retention time=1.8 min; ESI-MS(+) m/z [M+2H]²⁺: 1189.2.

### Preparation of Example 2448

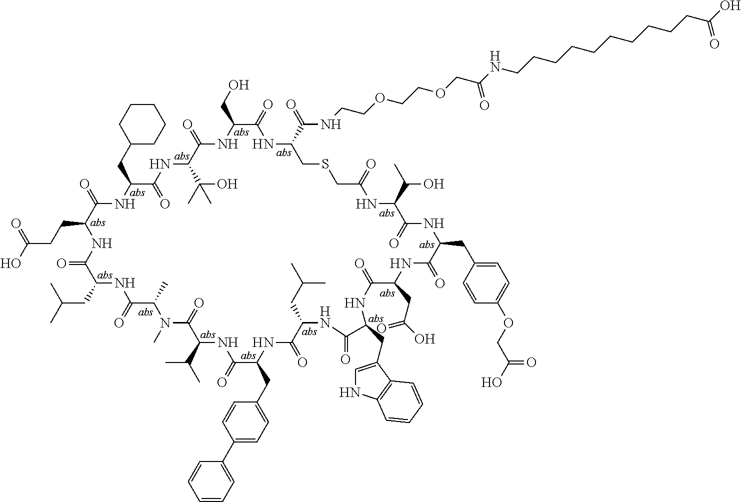 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2448 was prepared on a 50 μmol scale. The yield of the product was 20.5 mg, and its estimated purity by LCMS analysis was 95.8%. Analysis condition B: Retention time=2.26 min; ESI-MS(+) m/z [M+2H]²⁺: 1117.1.

### Preparation of Example 2449

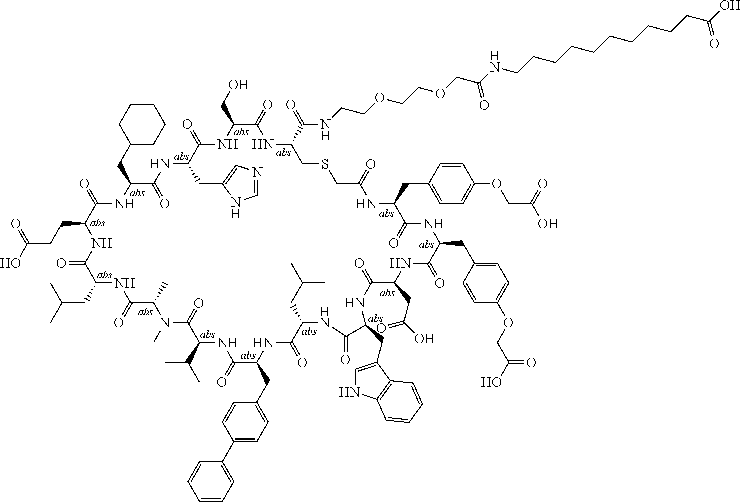 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2449 was prepared on a 50 μmol scale. The yield of the product was 19 mg, and its estimated purity by LCMS analysis was 97.5%. Analysis condition B: Retention time=2.18 min; ESI-MS(+) m/z [M+2H]²⁺: 792.1.

### Preparation of Example 2450

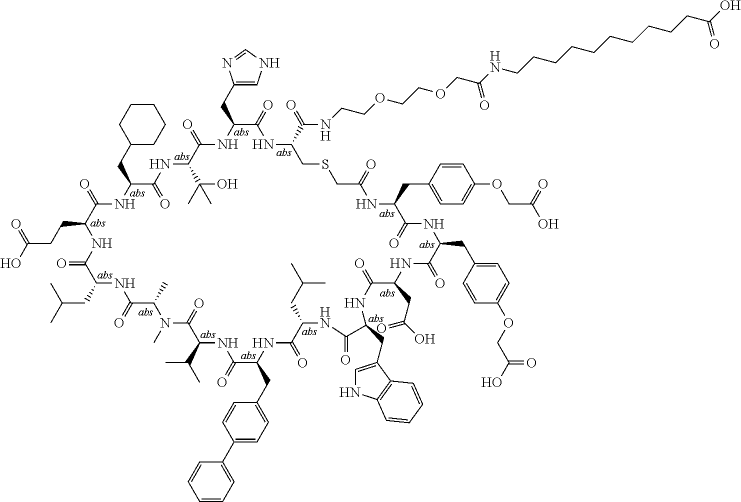 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCC(=O)O)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2450 was prepared on a 50 μmol scale. The yield of the product was 18.1 mg, and its estimated purity by LCMS analysis was 95.8%. Analysis condition A: Retention time=1.52 min; ESI-MS(+) m/z [M+3H]³⁺: 799.2.

### Preparation of Example 2451

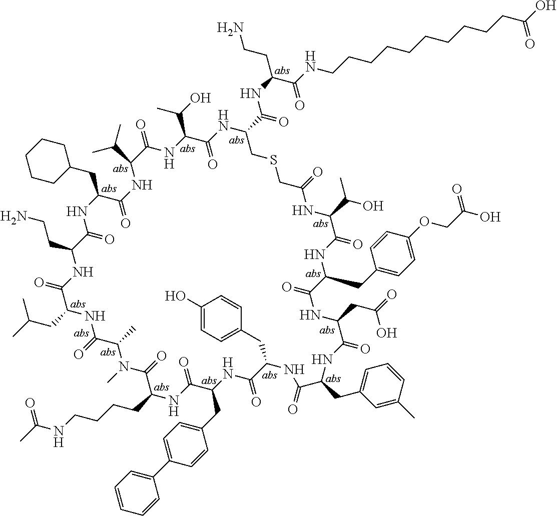 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2451 was prepared on a 50 μmol scale. The yield of the product was 37.2 mg, and its estimated purity by LCMS analysis was 96.7%. Analysis condition B: Retention time=1.82 min; ESI-MS(+) m/z [M+2H]²⁺: 1126.2.

### Preparation of Example 2452

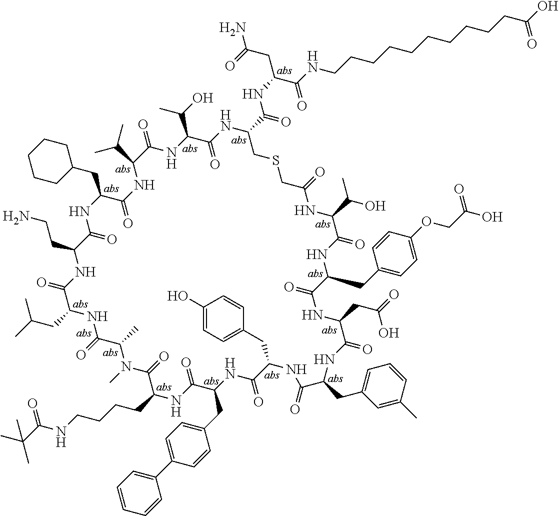 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCCNC(=O)C(C)(C)C)NC(=O)[C@H](Cc3ccc(-c4ccccc4)cc3)NC(=O)[C@H](Cc3ccc(O)cc3)NC2=O)c1]

Example 2452 was prepared on a 50 μmol scale. The yield of the product was 27 mg, and its estimated purity by LCMS analysis was 98.3%. Analysis condition B: Retention time=1.99 min; ESI-MS(+) m/z [M+2H]²⁺: 1154.2.

### Preparation of Example 2453

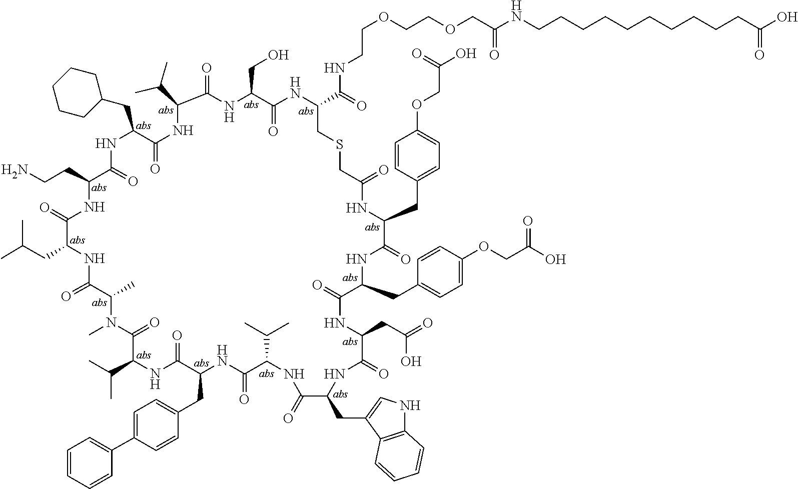 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Example 2453 was prepared on a 50 μmol scale. The yield of the product was 24.4 mg, and its estimated purity by LCMS analysis was 95.1%. Analysis condition A: Retention time=1.68 min; ESI-MS(+) m/z [M+2H]²⁺: 1147.2.

### Preparation of Example 2454

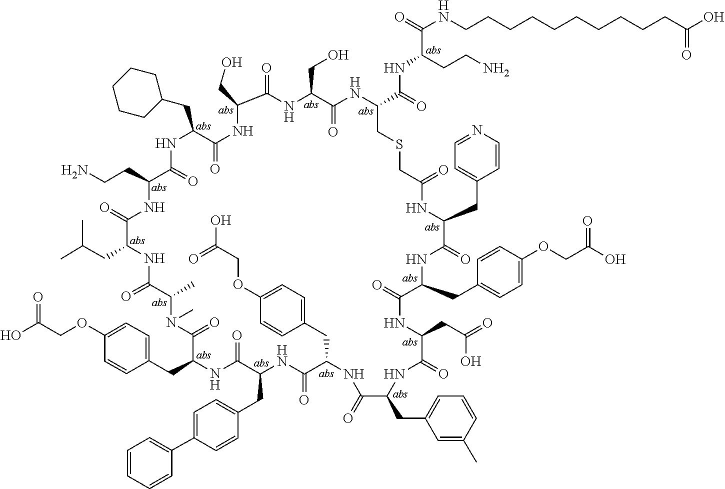 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](CO)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2454 was prepared on a 50 μmol scale. The yield of the product was 3.2 mg, and its estimated purity by LCMS analysis was 96.3%. Analysis condition A: Retention time=1.48 min; ESI-MS(+) m/z [M+2H]²⁺: 1191.9.

### Preparation of Example 2455

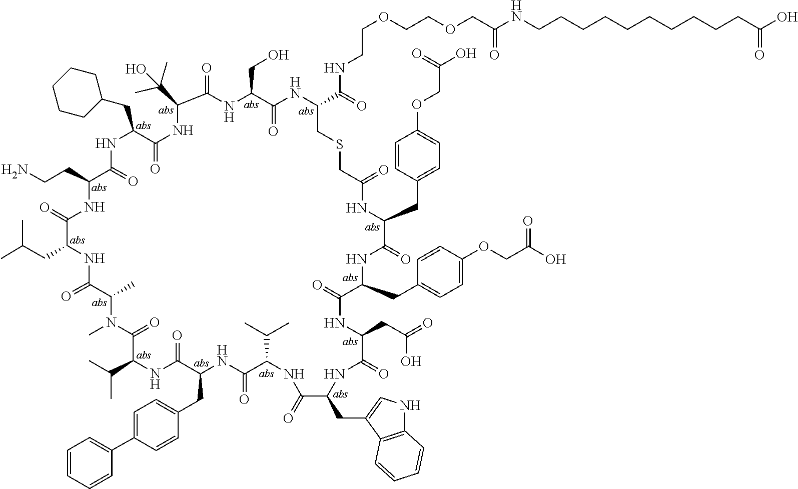 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2455 was prepared on a 50 μmol scale. The yield of the product was 29.4 mg, and its estimated purity by LCMS analysis was 100%. Analysis condition A: Retention time=1.68 min; ESI-MS(+) m/z [M+2H]²⁺: 1155.

### Preparation of Example 2456

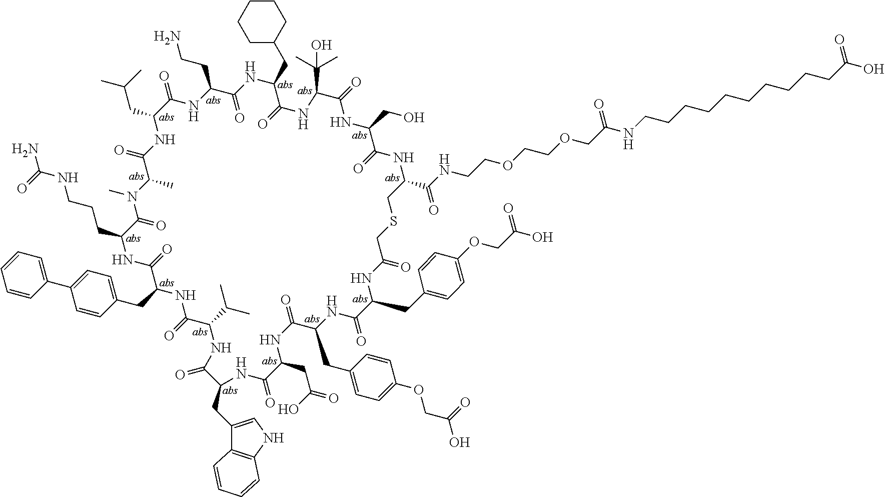 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2456 was prepared on a 50 μmol scale. The yield of the product was 29.9 mg, and its estimated purity by LCMS analysis was 97.5%. Analysis condition A: Retention time=1.63 min; ESI-MS(+) m/z [M+2H]²⁺: 1184.1.

### Preparation of Example 2457

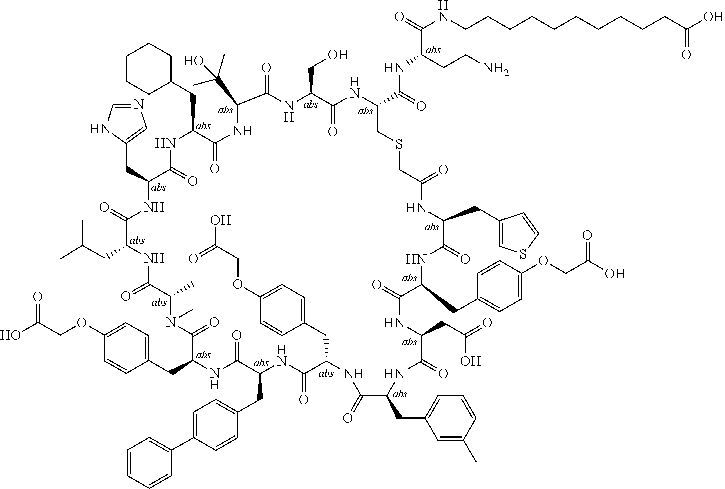 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccsc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2457 was prepared on a 50 μmol scale. The yield of the product was 6.9 mg, and its estimated purity by LCMS analysis was 97.5%. Analysis condition A: Retention time=1.56 min; ESI-MS(+) m/z [M+2H]²⁺: 817.8.

### Preparation of Example 2458

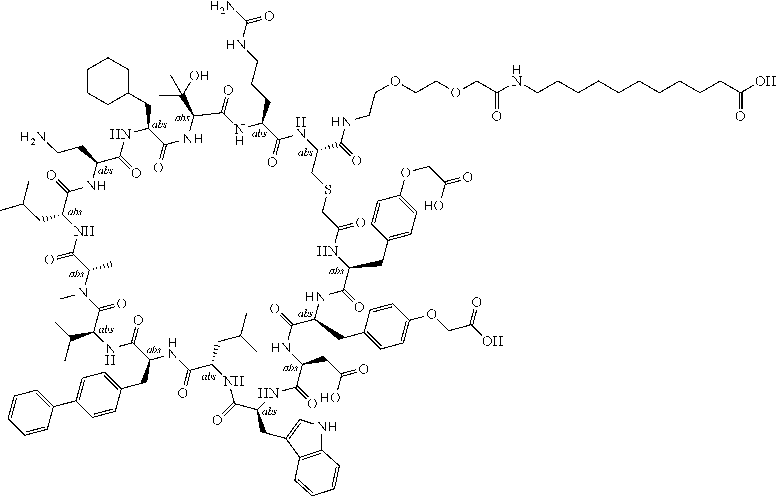 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC1=O]

Example 2458 was prepared on a 50 μmol scale. The yield of the product was 20.6 mg, and its estimated purity by LCMS analysis was 95%. Analysis condition B: Retention time=2.02 min; ESI-MS(+) m/z [M+2H]²⁺: 1197.1.

### Preparation of Example 2459

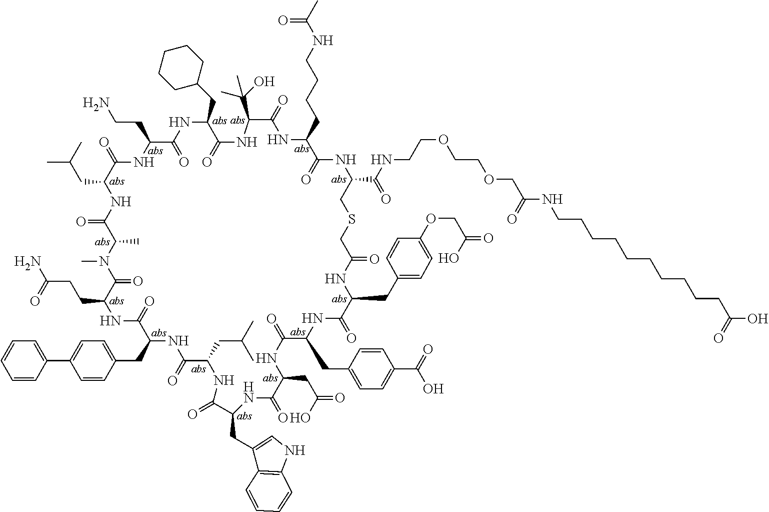 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC1=O]

Example 2459 was prepared on a 50 μmol scale. The yield of the product was 34.9 mg, and its estimated purity by LCMS analysis was 100%. Analysis condition A: Retention time=1.68 min; ESI-MS(+) m/z [M+2H]²⁺: 1203.1.

### Preparation of Example 2460

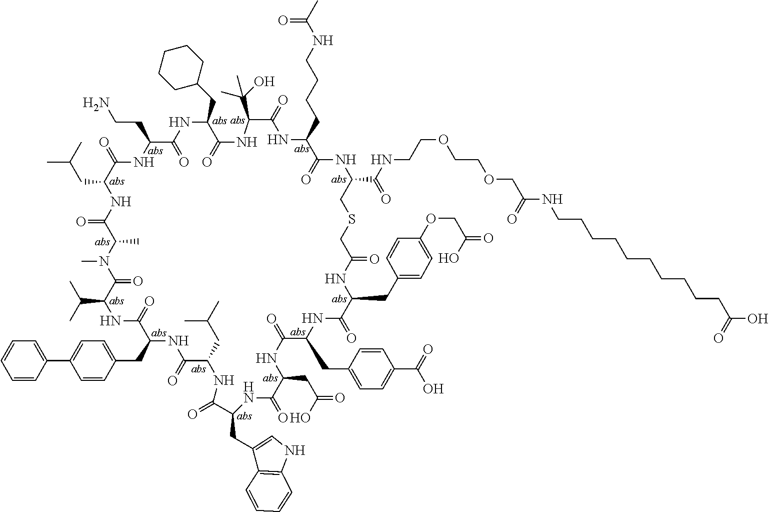 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC1=O]

Example 2460 was prepared on a 50 μmol scale. The yield of the product was 28.8 mg, and its estimated purity by LCMS analysis was 95.2%. Analysis condition A: Retention time=1.77 min; ESI-MS(+) m/z [M+2H]²⁺: 1188.2.

### Preparation of Example 2461

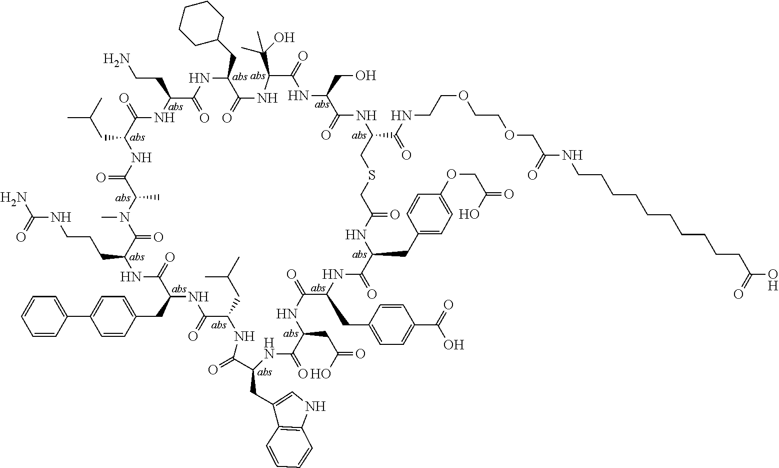 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC1=O]

Example 2461 was prepared on a 50 μmol scale. The yield of the product was 23.3 mg, and its estimated purity by LCMS analysis was 96.3%. Analysis condition A: Retention time=1.63 min; ESI-MS(+) m/z [M+2H]²⁺: 1169.2.

### Preparation of Example 2462

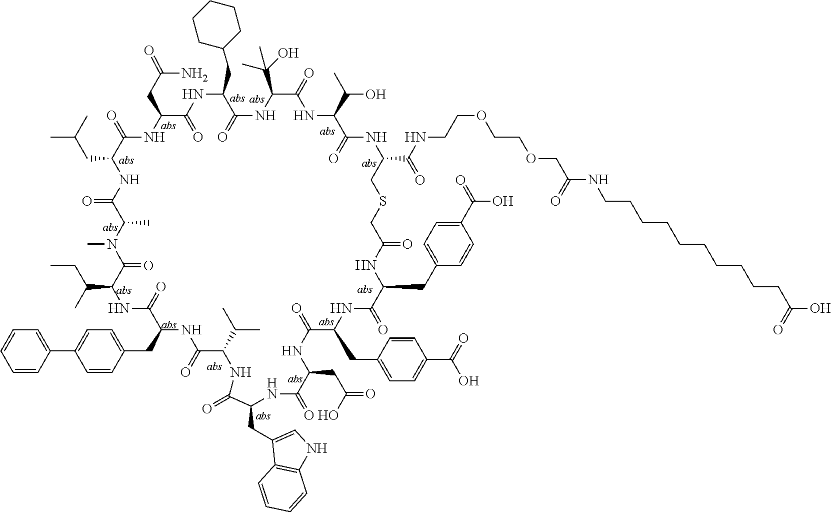 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(C(=O)O)cc2)NC(=O)CSC[C@@H](C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CC(N)=O)NC(=O)[C@@H](CC(C)C)NC(=O)[C@H](C)N(C)C1=O]

Example 2462 was prepared on a 50 μmol scale. The yield of the product was 6.1 mg, and its estimated purity by LCMS analysis was 99.4%. Analysis condition B: Retention time=2.28 min; ESI-MS(+) m/z [M+2H]²⁺: 1139.2.

### Preparation of Example 2463

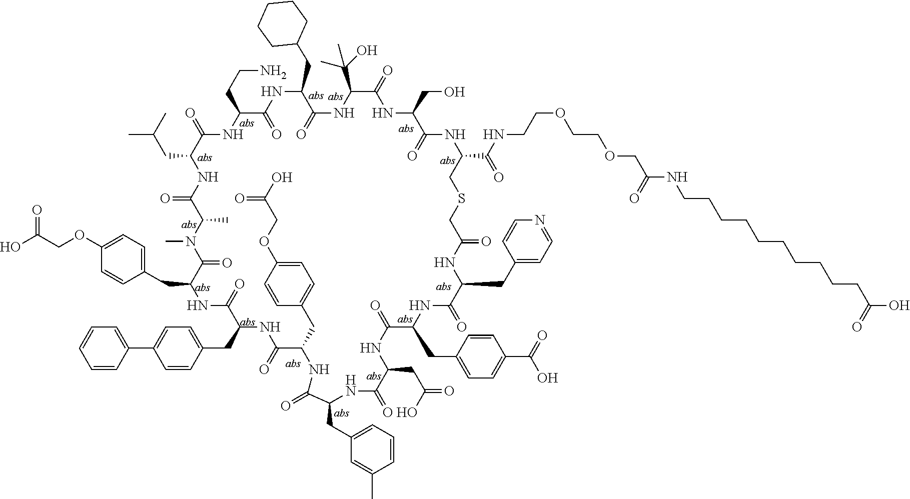 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2463 was prepared on a 50 μmol scale. The yield of the product was 21.5 mg, and its estimated purity by LCMS analysis was 98.5%. Analysis condition B: Retention time=1.9 min; ESI-MS(+) m/z [M+2H]²⁺: 1213.2.

### Preparation of Example 2464

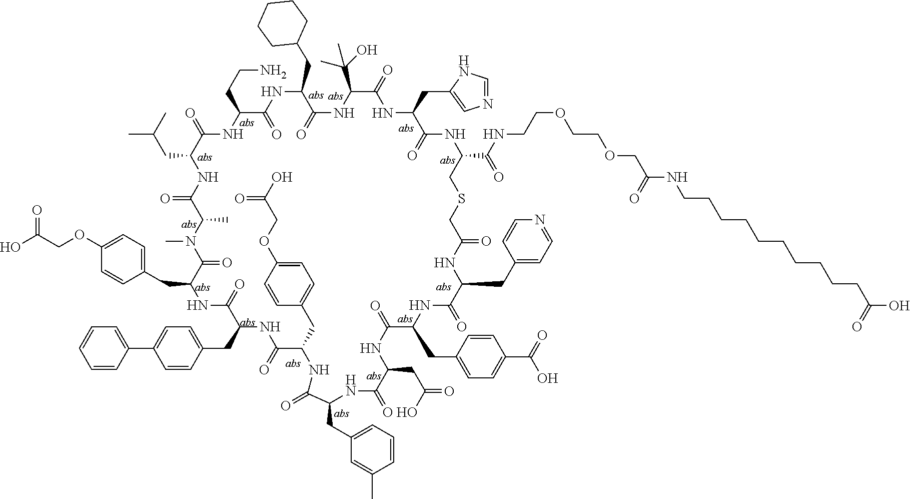 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2464 was prepared on a 50 μmol scale. The yield of the product was 31 mg, and its estimated purity by LCMS analysis was 95.3%. Analysis condition B: Retention time=1.77 min; ESI-MS(+) m/z [M+2H]²⁺: 1238.2.

### Preparation of Example 2465

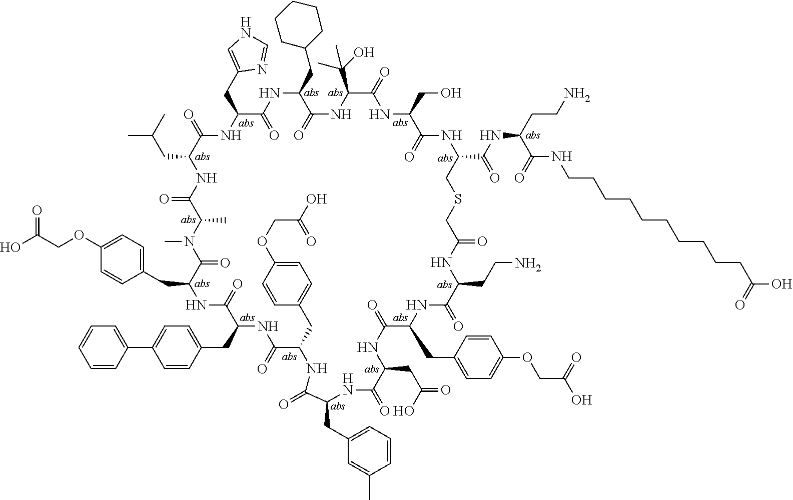 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2465 was prepared on a 50 μmol scale. The yield of the product was 3.8 mg, and its estimated purity by LCMS analysis was 95.5%. Analysis condition A: Retention time=1.52 min; ESI-MS(+) m/z [M+2H]²⁺: 1199.9.

### Preparation of Example 2466

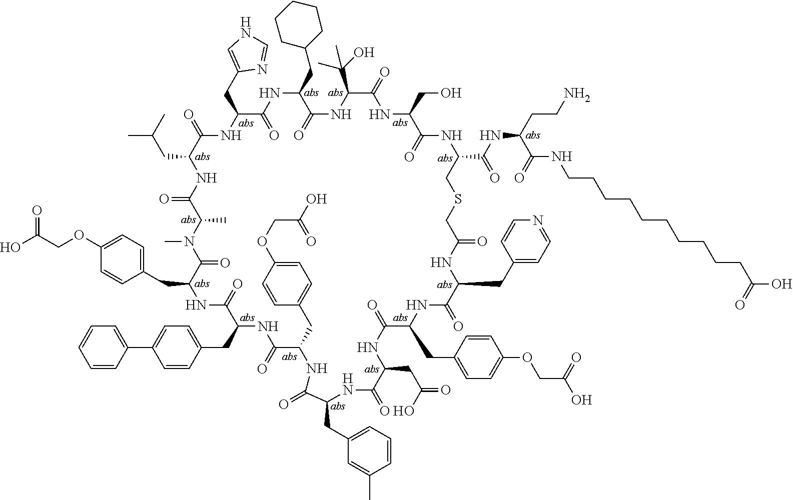

Example 2466 was prepared on a 50 μmol scale. The yield of the product was 5.7 mg, and its estimated purity by LCMS analysis was 95.4%. Analysis condition A: Retention time=1.48, 1.5 min; ESI-MS(+) m/z [M+2H]²⁺: 1223.

### Preparation of Example 2467

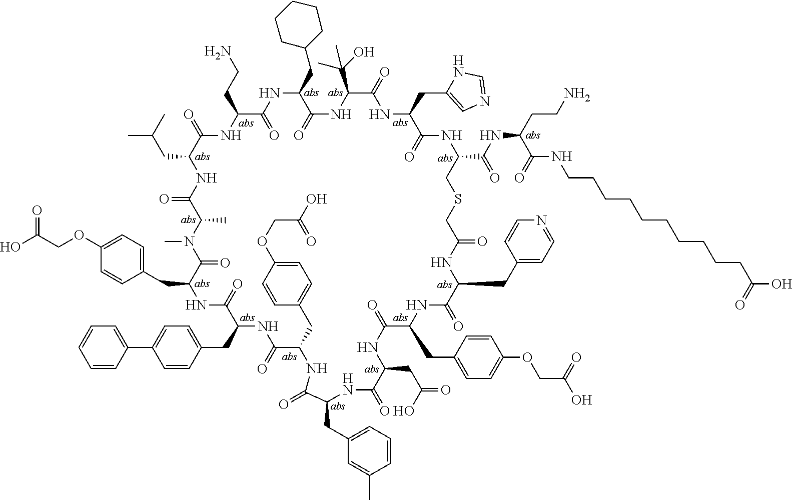 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2467 was prepared on a 50 μmol scale. The yield of the product was 12.8 mg, and its estimated purity by LCMS analysis was 95.8%. Analysis condition A: Retention time=1.44 min; ESI-MS(+) m/z [M+2H]²⁺: 1230.1.

### Preparation of Example 2468

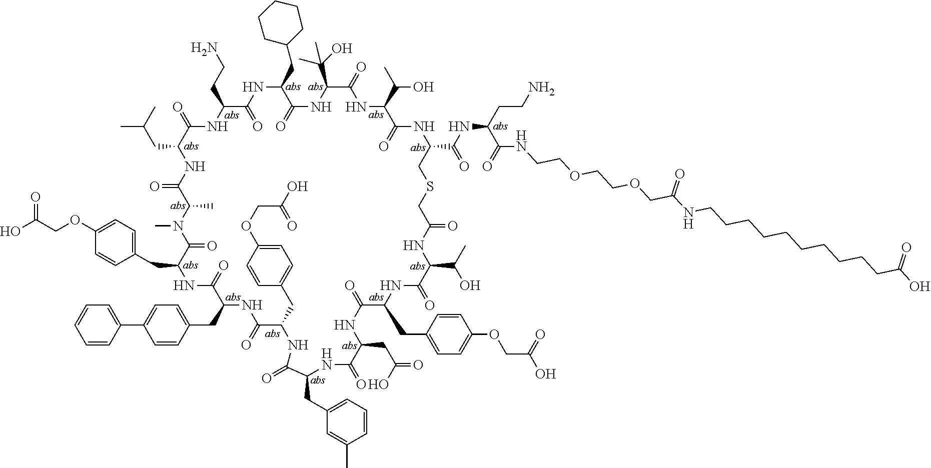 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2468 was prepared on a 50 μmol scale. The yield of the product was 43.1 mg, and its estimated purity by LCMS analysis was 95.8%. Analysis condition B: Retention time=1.93 min; ESI-MS(+) m/z [M+2H]²⁺: 1261.5.

### Preparation of Example 2469

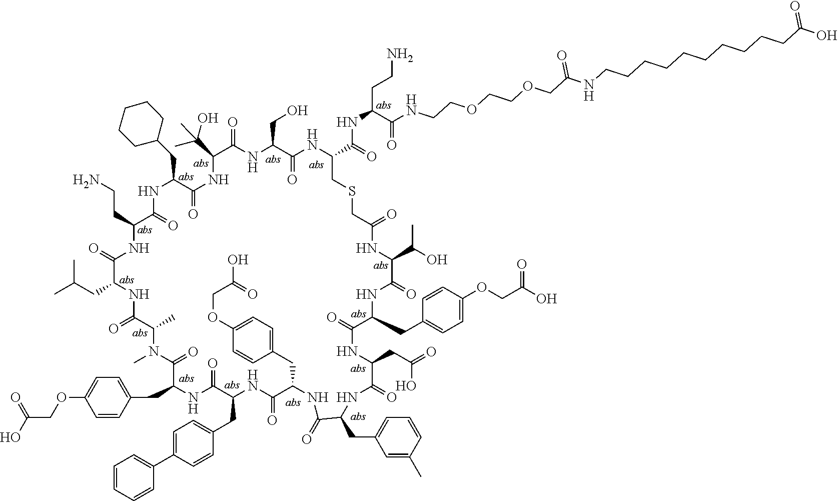 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2469 was prepared on a 50 μmol scale. The yield of the product was 17.4 mg, and its estimated purity by LCMS analysis was 100%. Analysis condition A: Retention time=1.5 min; ESI-MS(+) m/z [M+2H]²⁺: 1254.3.

### Preparation of Example 2470

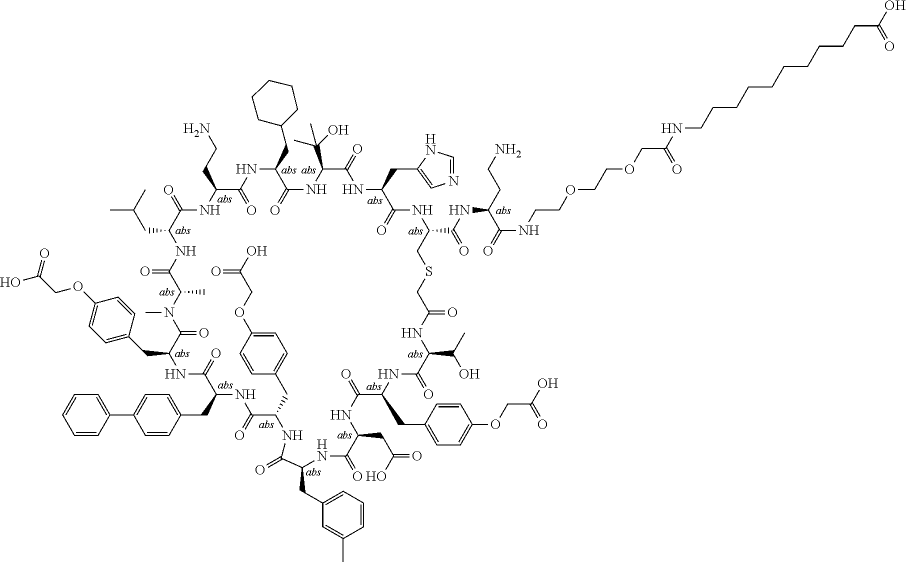 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2470 was prepared on a 50 μmol scale. The yield of the product was 28.1 mg, and its estimated purity by LCMS analysis was 95.7%. Analysis condition B: Retention time=1.77 min; ESI-MS(+) m/z [M+2H]²⁺: 1279.3.

### Preparation of Example 2471

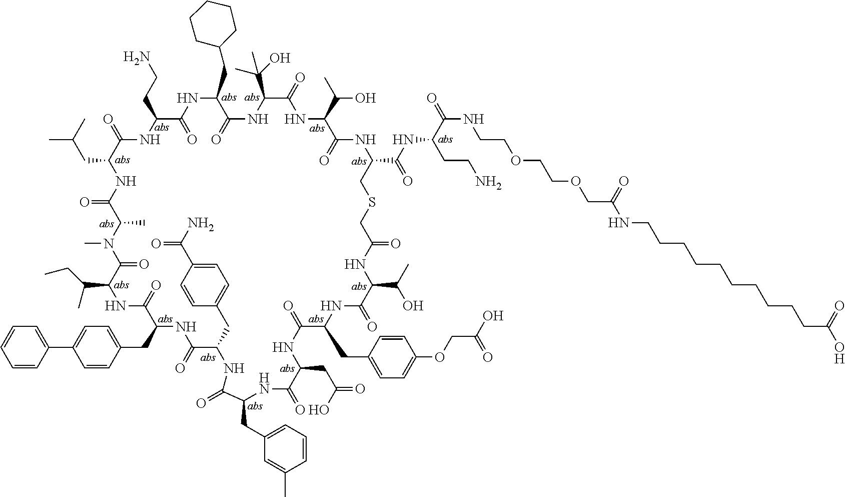 [SMILES: CCC(C)[C@@H]1NC(=O)[C@H](Cc2ccc(-c3ccccc3)cc2)NC(=O)[C@H](Cc2ccc(C(N)=O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2471 was prepared on a 50 μmol scale. The yield of the product was 15.9 mg, and its estimated purity by LCMS analysis was 96.6%. Analysis condition A: Retention time=1.73 min; ESI-MS(+) m/z [M+2H]²⁺: 1192.2.

### Preparation of Example 2472

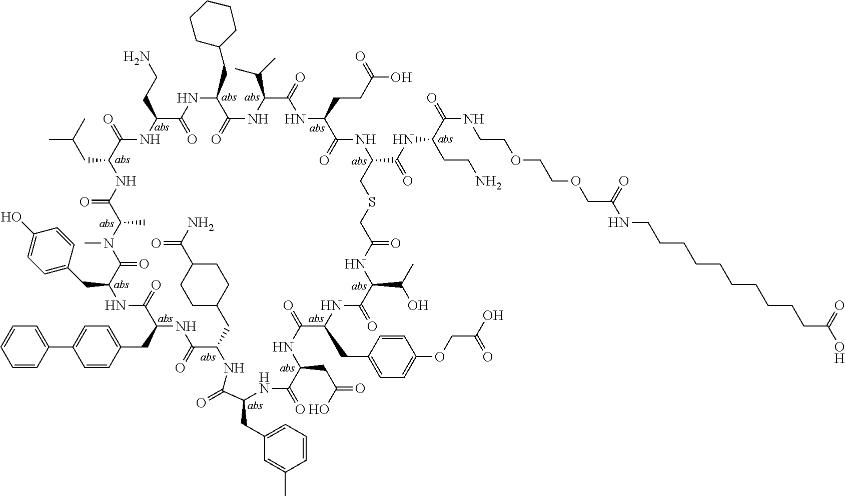 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](CC3CCC(C(N)=O)CC3)NC2=O)c1]

Example 2472 was prepared on a 50 μmol scale. The yield of the product was 12.1 mg, and its estimated purity by LCMS analysis was 95.5%. Analysis condition B: Retention time=1.89 min; ESI-MS(+) m/z [M+2H]²⁺: 1223.2.

### Preparation of Example 2473

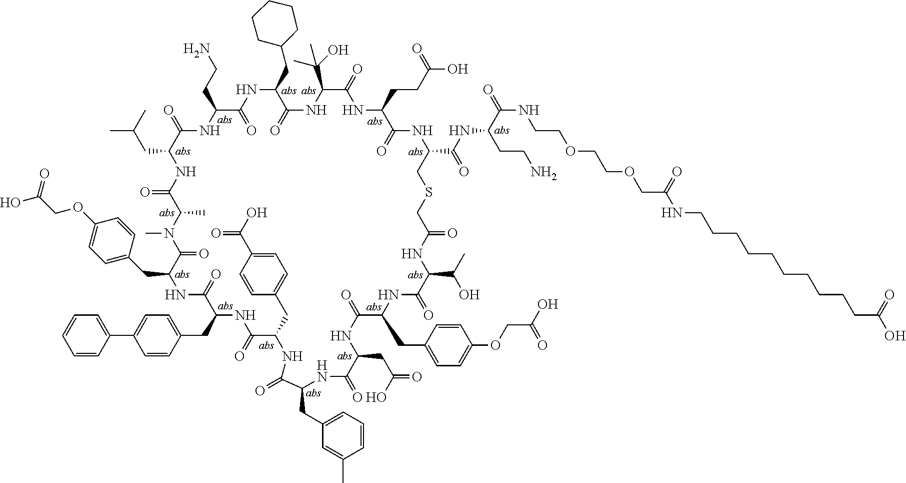 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2473 was prepared on a 50 μmol scale. The yield of the product was 22.4 mg, and its estimated purity by LCMS analysis was 93.8%. Analysis condition B: Retention time=1.92 min; ESI-MS(+) m/z [M+2H]²⁺: 1260.4.

### Preparation of Example 2474

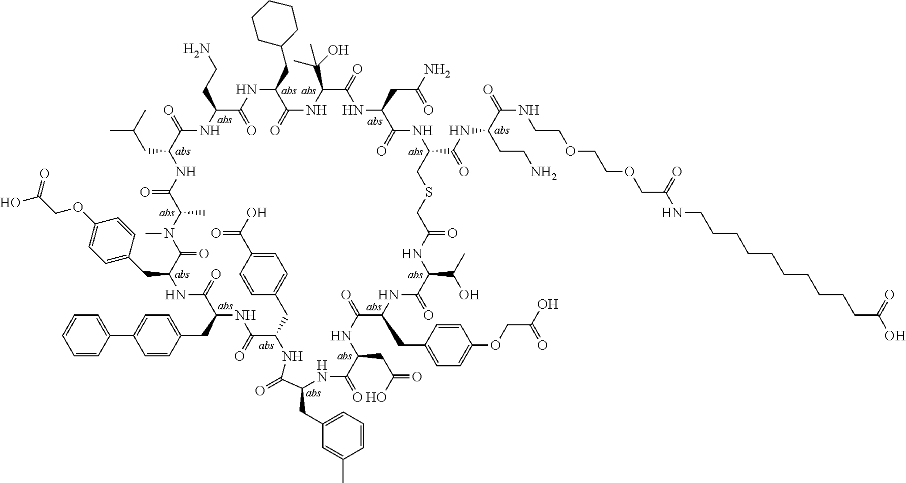 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2474 was prepared on a 50 μmol scale. The yield of the product was 34 mg, and its estimated purity by LCMS analysis was 97.1%. Analysis condition A: Retention time=1.46 min; ESI-MS(+) m/z [M+2H]²⁺: 1253.2.

### Preparation of Example 2475

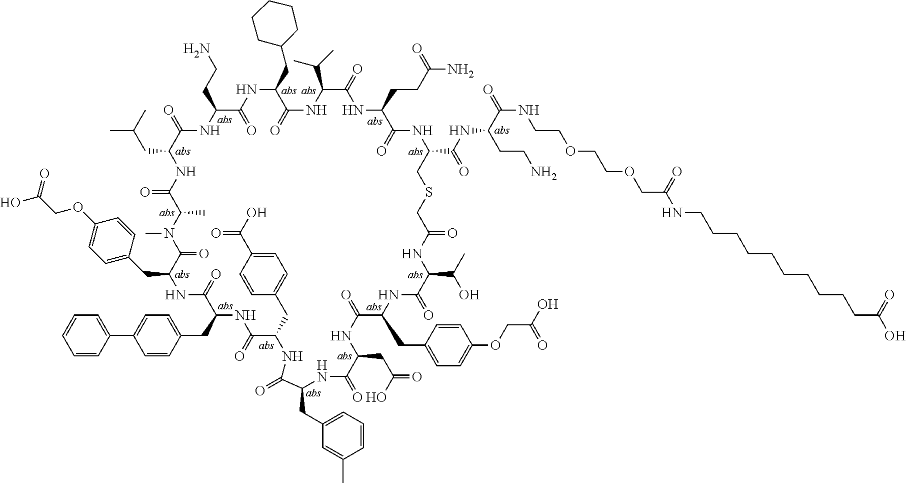 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2475 was prepared on a 50 μmol scale. The yield of the product was 29.5 mg, and its estimated purity by LCMS analysis was 100%. Analysis condition A: Retention time=1.48 min; ESI-MS(+) m/z [M+2H]²⁺: 1252.1.

### Preparation of Example 2476

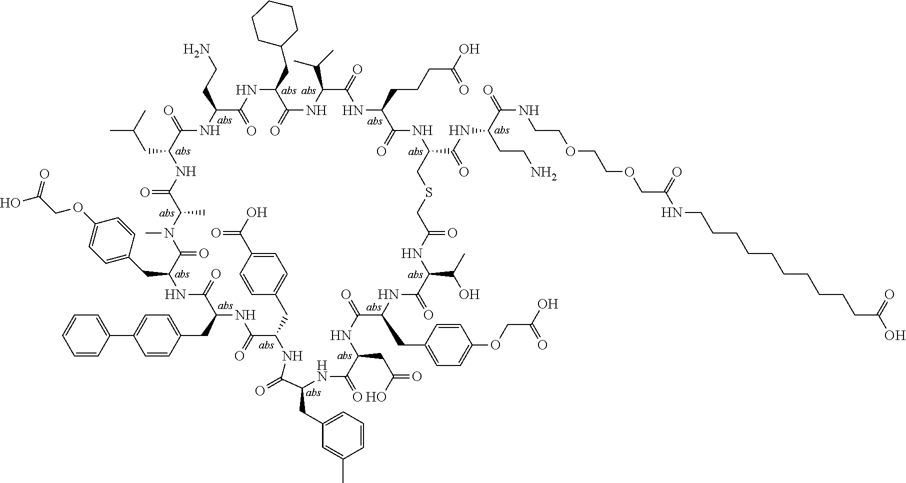 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2476 was prepared on a 50 μmol scale. The yield of the product was 18.5 mg, and its estimated purity by LCMS analysis was 96.2%. Analysis condition A: Retention time=1.33 min; ESI-MS(+) m/z [M+2H]²⁺: 1259.2.

### Preparation of Example 2477

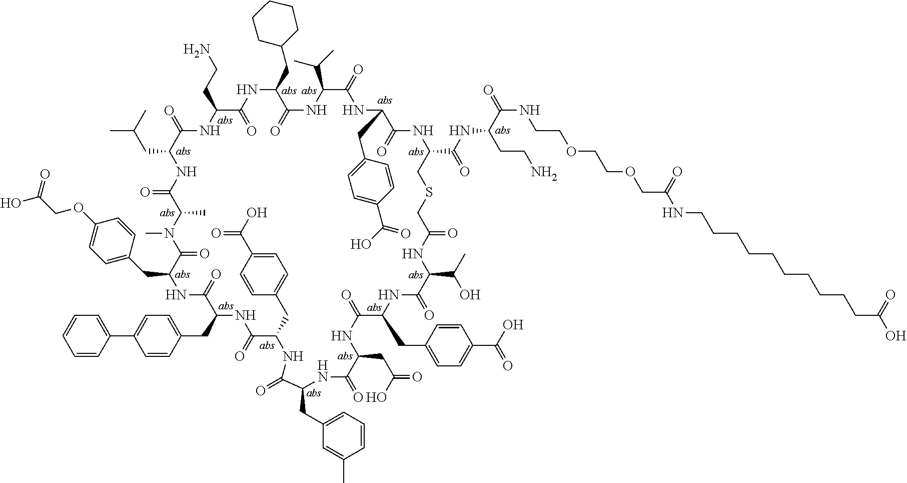 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](Cc3ccc(C(=O)O)cc3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2477 was prepared on a 50 μmol scale. The yield of the product was 11.8 mg, and its estimated purity by LCMS analysis was 100%. Analysis condition A: Retention time=1.41 min; ESI-MS(+) m/z [M+2H]²⁺: 1268.

### Preparation of Example 2478

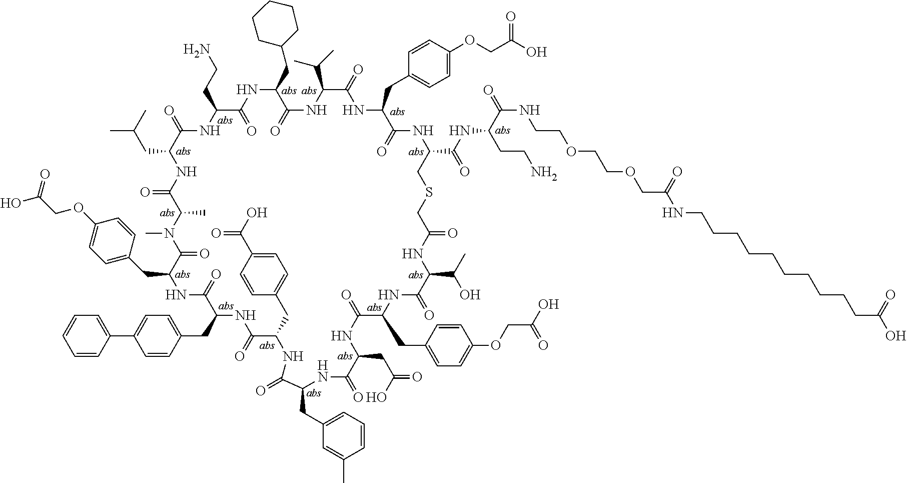 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](Cc3ccc(OCC(=O)O)cc3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2478 was prepared on a 50 μmol scale. The yield of the product was 35.9 mg, and its estimated purity by LCMS analysis was 100%. Analysis condition B: Retention time=1.92 min; ESI-MS(+) m/z [M+3H]³⁺: 866.0.

### Preparation of Example 2479

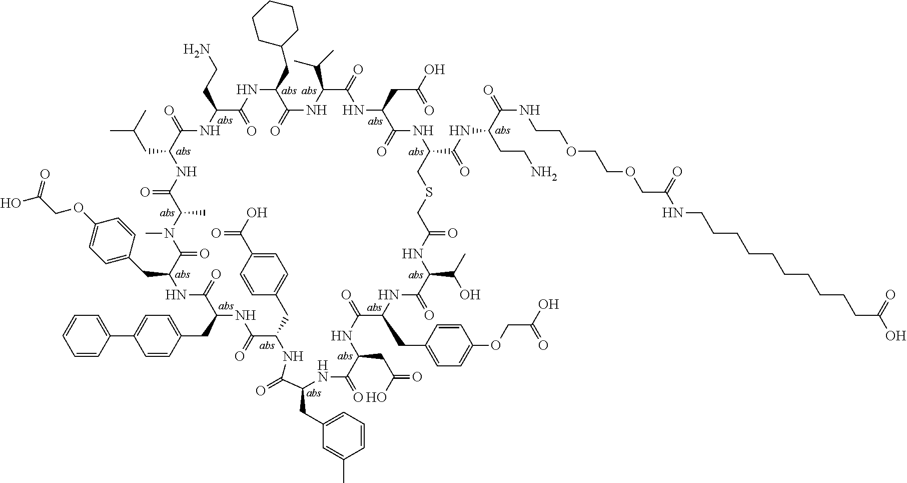 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2479 was prepared on a 50 μmol scale. The yield of the product was 20 mg, and its estimated purity by LCMS analysis was 95.8%. Analysis condition B: Retention time=1.91 min; ESI-MS(+) m/z [M+2H]²⁺: 1245.3.

### Preparation of Example 2480

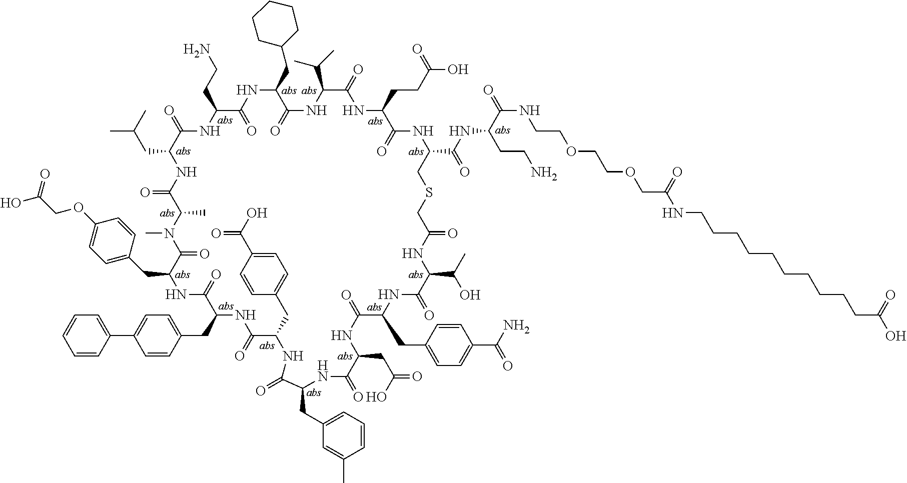 [SMILES: Cc1cccc(C[C@@H]2NC(=O)[C@H](CC(=O)O)NC(=O)[C@H](Cc3ccc(C(N)=O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2480 was prepared on a 50 μmol scale. The yield of the product was 19.3 mg, and its estimated purity by LCMS analysis was 100%. Analysis condition A: Retention time=1.49 min; ESI-MS(+) m/z [M+2H]²⁺: 1237.1.

### Preparation of Example 2481

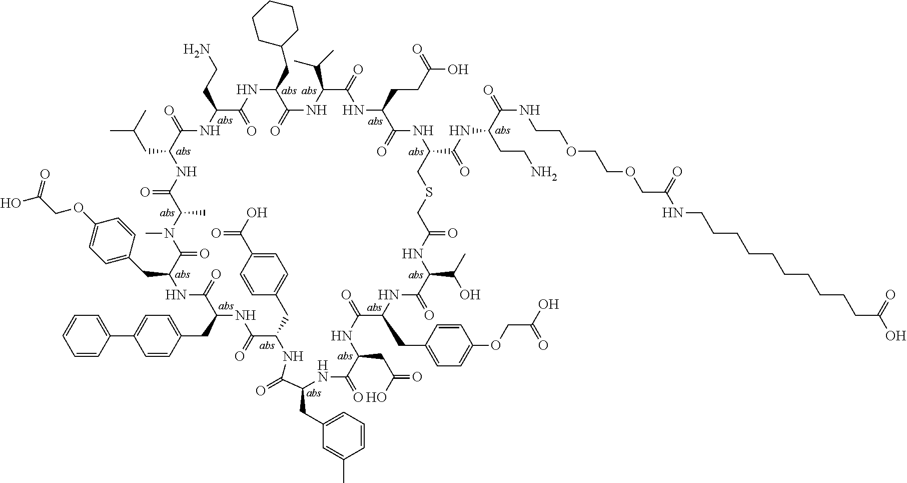 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2481 was prepared on a 50 μmol scale. The yield of the product was 13.1 mg, and its estimated purity by LCMS analysis was 96.4%. Analysis condition A: Retention time=1.43 min; ESI-MS(+) m/z [M+3H]³⁺: 835.3.

### Preparation of Example 2482

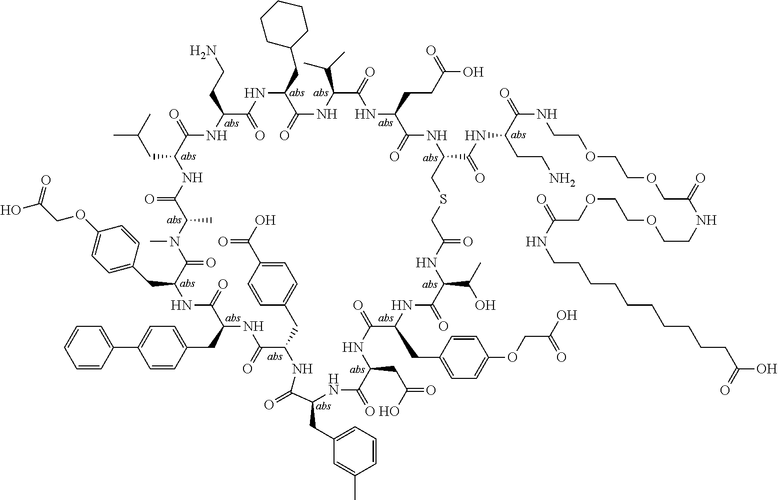 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2482 was prepared on a 50 μmol scale. The yield of the product was 17.9 mg, and its estimated purity by LCMS analysis was 98.7%. Analysis condition A: Retention time=1.49 min; ESI-MS(+) m/z [M+2H]²⁺: 1324.9.

### Preparation of Example 2483

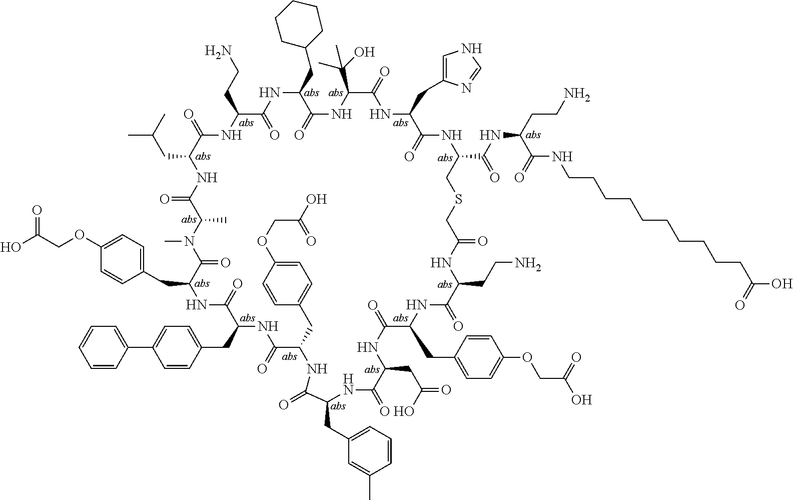 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2483 was prepared on a 50 μmol scale. The yield of the product was 21.1 mg, and its estimated purity by LCMS analysis was 100%. Analysis condition A: Retention time=1.74 min; ESI-MS(+) m/z [M+2H]²⁺: 1206.4.

### Preparation of Example 2484

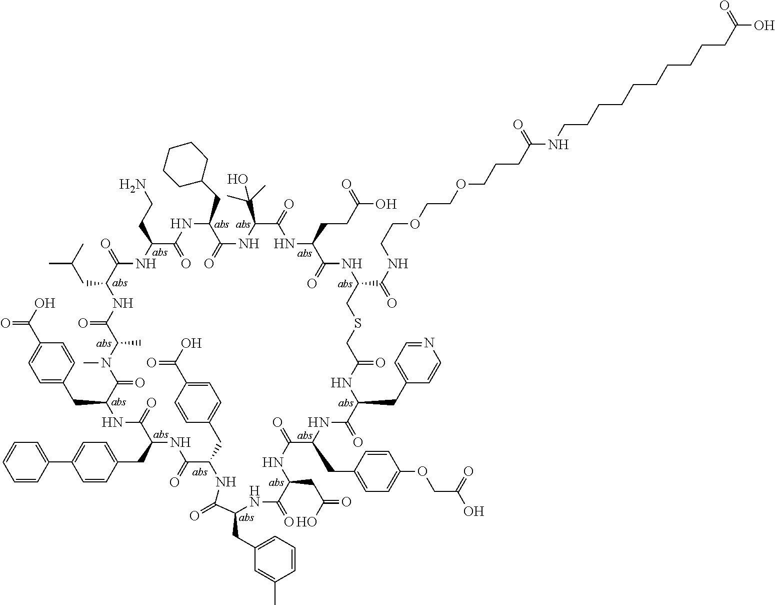 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)NCCOCCOCCCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2484 was prepared on a 50 μmol scale. The yield of the product was 22.9 mg, and its estimated purity by LCMS analysis was 100%. Analysis condition A: Retention time=1.44 min; ESI-MS(+) m/z [M+2H]²⁺: 1219.1.

### Preparation of Example 2485

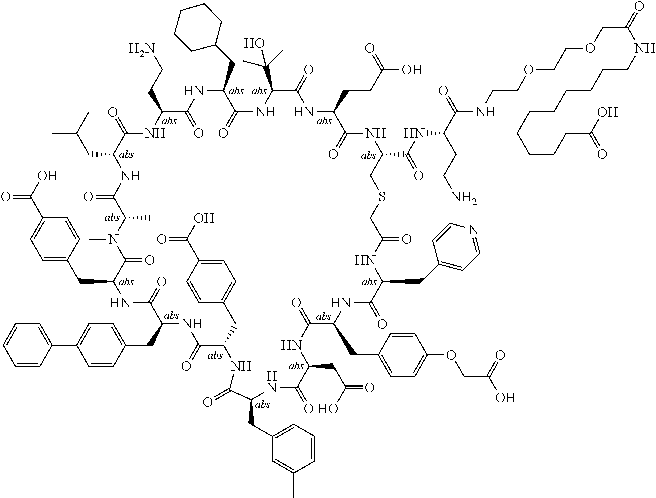 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2485 was prepared on a 50 μmol scale. The yield of the product was 61 mg, and its estimated purity by LCMS analysis was 96.5%. Analysis condition A: Retention time=1.42 min; ESI-MS(+) m/z [M+2H]²⁺: 1269.1.

### Preparation of Example 2486

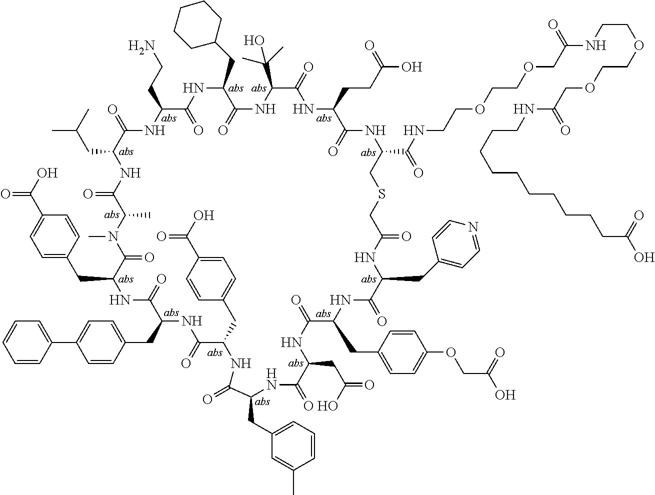 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2486 was prepared on a 50 μmol scale. The yield of the product was 28.4 mg, and its estimated purity by LCMS analysis was 100%. Analysis condition A: Retention time=1.43 min; ESI-MS(+) m/z [M+2H]²⁺: 1291.1.

### Preparation of Example 2487

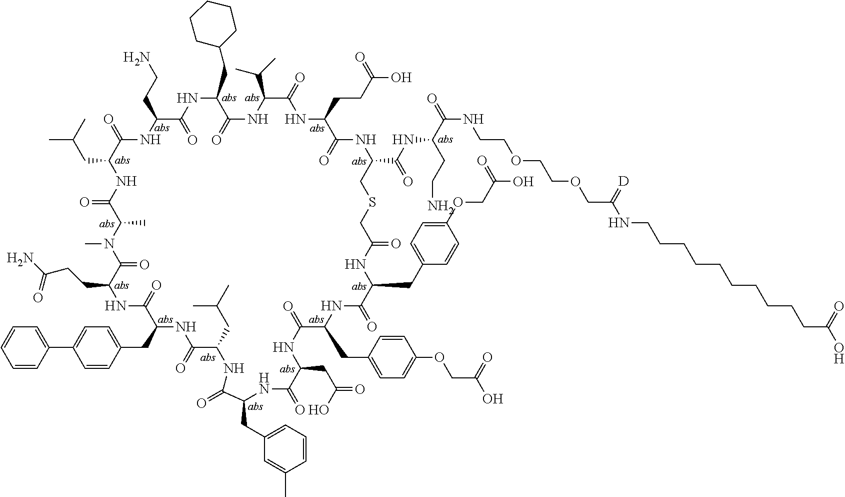

Example 2487 was prepared on a 50 μmol scale. The yield of the product was 24.4 mg, and its estimated purity by LCMS analysis was 96.1%. Analysis condition A: Retention time=1.64 min; ESI-MS(+) m/z [M+2H]²⁺: 1239.9.

### Preparation of Example 2488

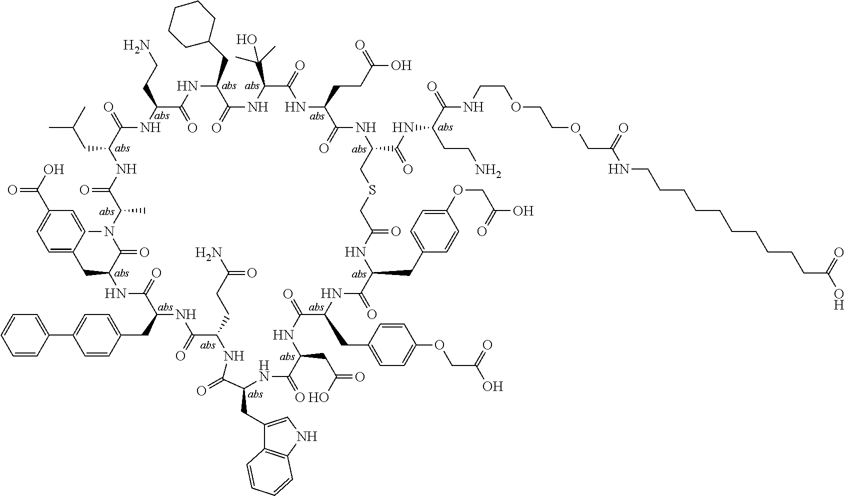 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](CCC(N)=O)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2488 was prepared on a 50 μmol scale. The yield of the product was 61.7 mg, and its estimated purity by LCMS analysis was 90.4%. Analysis condition A: Retention time=1.33 min; ESI-MS(+) m/z [M+2H]²⁺: 1286.2.

### Preparation of Example 2489

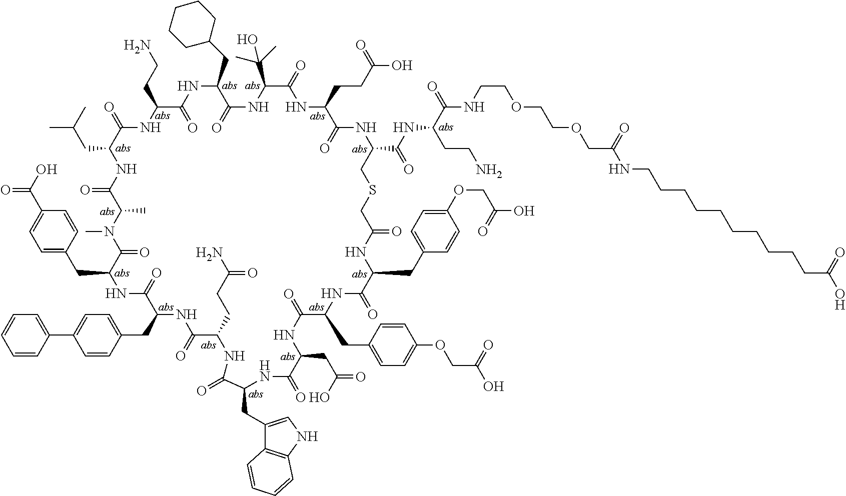 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](CCC(N)=O)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2489 was prepared on a 50 μmol scale. The yield of the product was 45.9 mg, and its estimated purity by LCMS analysis was 100%. Analysis condition A: Retention time=1.37 min; ESI-MS(+) m/z [M+2H]²⁺: 1287.1.

### Preparation of Example 2490

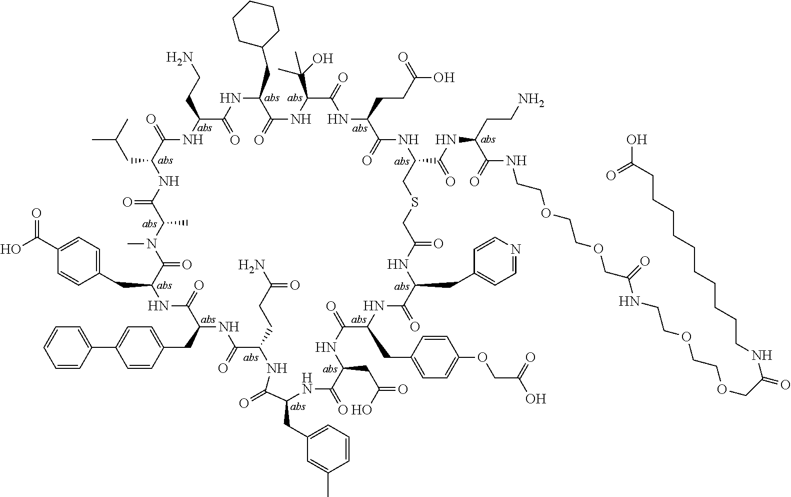 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](CCC(N)=O)NC2=O)c1]

Example 2490 was prepared on a 50 μmol scale. The yield of the product was 75.9 mg, and its estimated purity by LCMS analysis was 99.4%. Analysis condition B: Retention time=1.8 min; ESI-MS(+) m/z [M+3H]³⁺: 895.1.

### Preparation of Example 2491

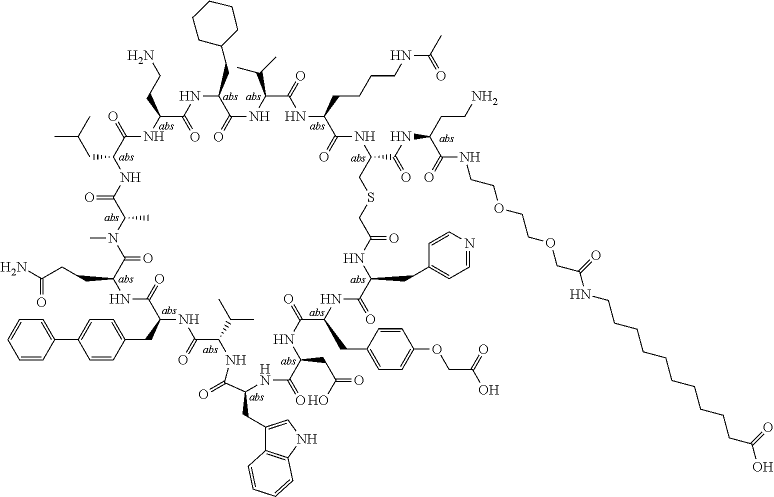 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC1=O]

Example 2491 was prepared on a 50 μmol scale. The yield of the product was 22.5 mg, and its estimated purity by LCMS analysis was 94.4%. Analysis condition A: Retention time=1.53, 1.57 min; ESI-MS(+) m/z [M+2H]²⁺: 1216.

### Preparation of Example 2492

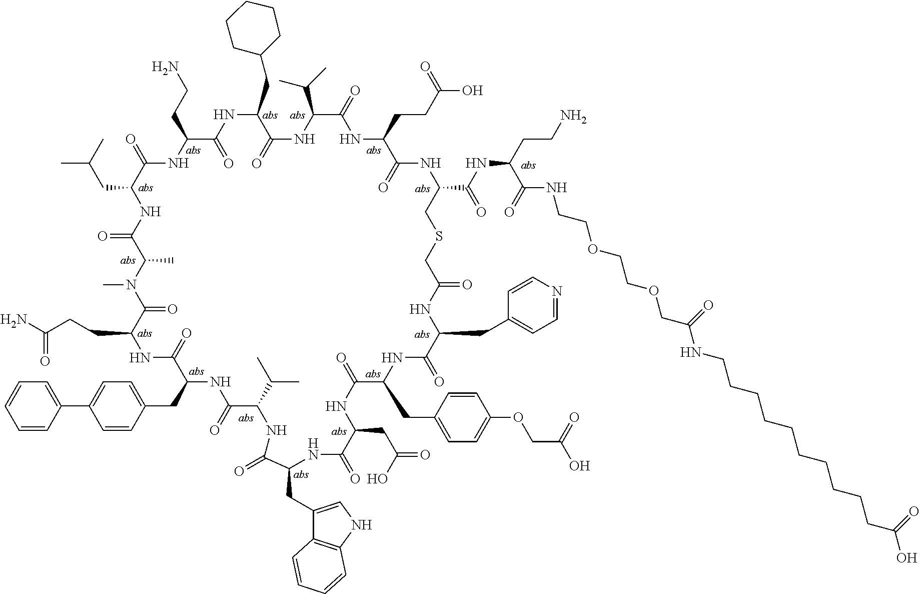 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2492 was prepared on a 50 μmol scale. The yield of the product was 17 mg, and its estimated purity by LCMS analysis was 92.6%. Analysis condition B: Retention time=1.67 min; ESI-MS(+) m/z [M+2H]²⁺: 1196.1.

### Preparation of Example 2493

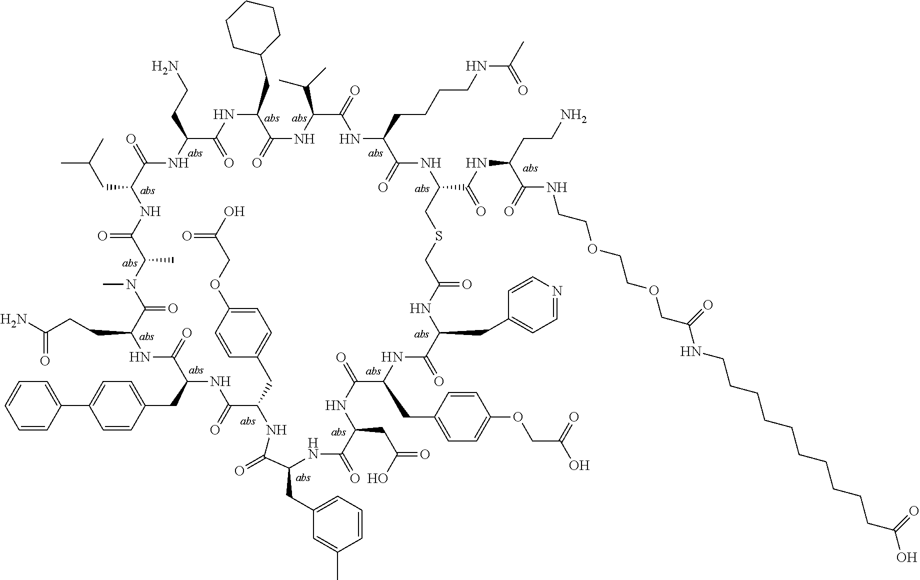 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC1=O]

Example 2493 was prepared on a 50 μmol scale. The yield of the product was 14.1 mg, and its estimated purity by LCMS analysis was 100%. Analysis condition B: Retention time=1.76 min; ESI-MS(+) m/z [M+3H]³⁺: 844.

### Preparation of Example 2494

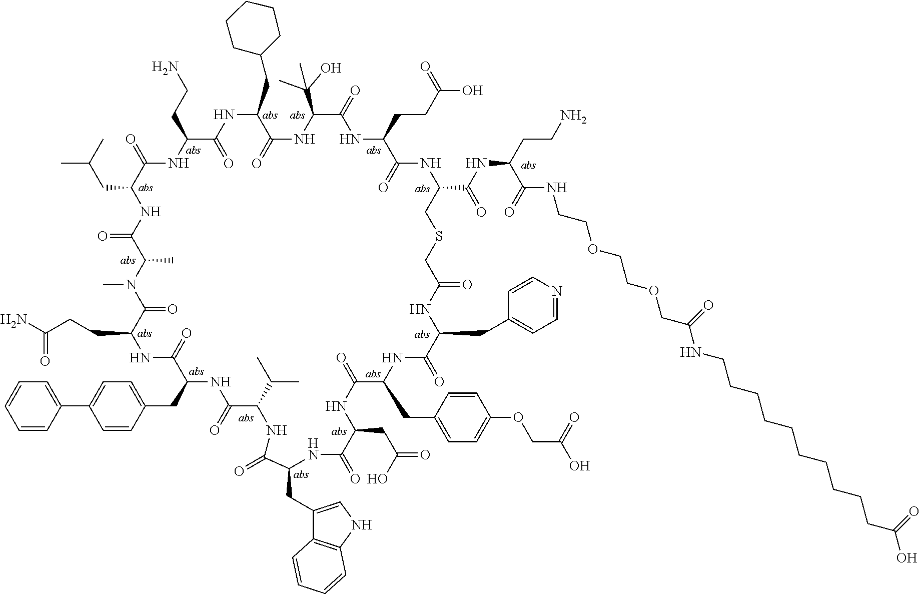 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2494 was prepared on a 50 μmol scale. The yield of the product was 63 mg, and its estimated purity by LCMS analysis was 96.1%. Analysis condition A: Retention time=1.56 min; ESI-MS(+) m/z [M+2H]²⁺: 1204.1.

### Preparation of Example 2495

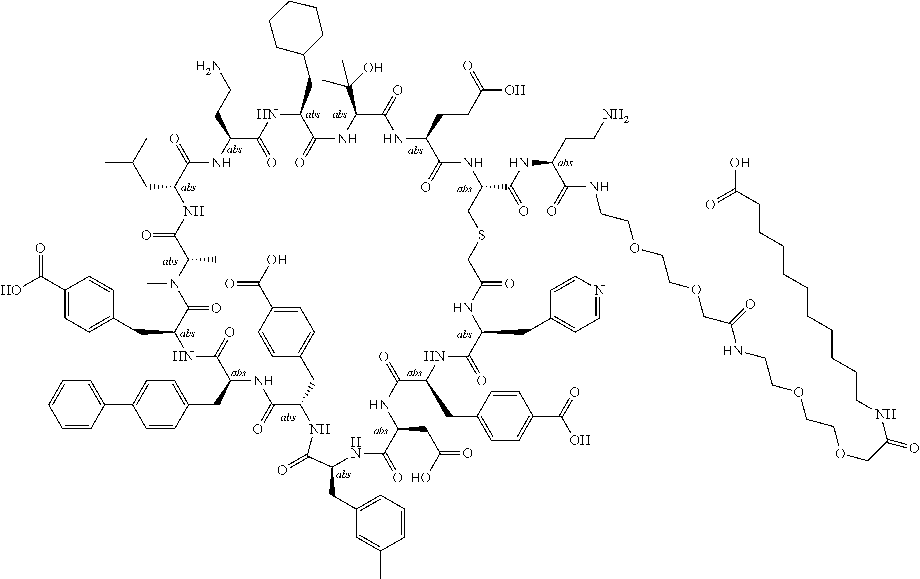 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2495 was prepared on a 50 μmol scale. The yield of the product was 94.5 mg, and its estimated purity by LCMS analysis was 97.6%. Analysis condition A: Retention time=1.46 min; ESI-MS(+) m/z [M+3H]³⁺: 885.

### Preparation of Example 2496

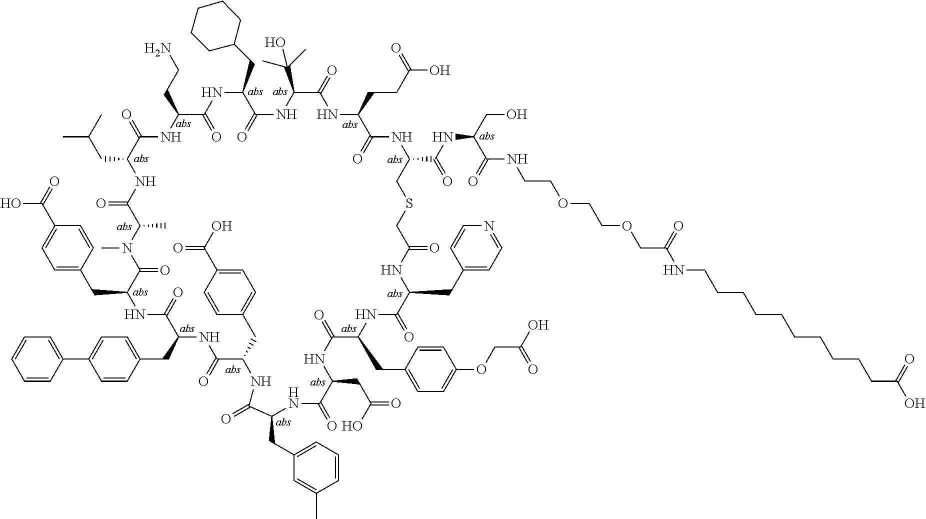 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CO)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2496 was prepared on a 50 μmol scale. The yield of the product was 15.4 mg, and its estimated purity by LCMS analysis was 100%. Analysis condition A: Retention time=1.42 min; ESI-MS(+) m/z [M+2H]²⁺: 1262.1.

### Preparation of Example 2497

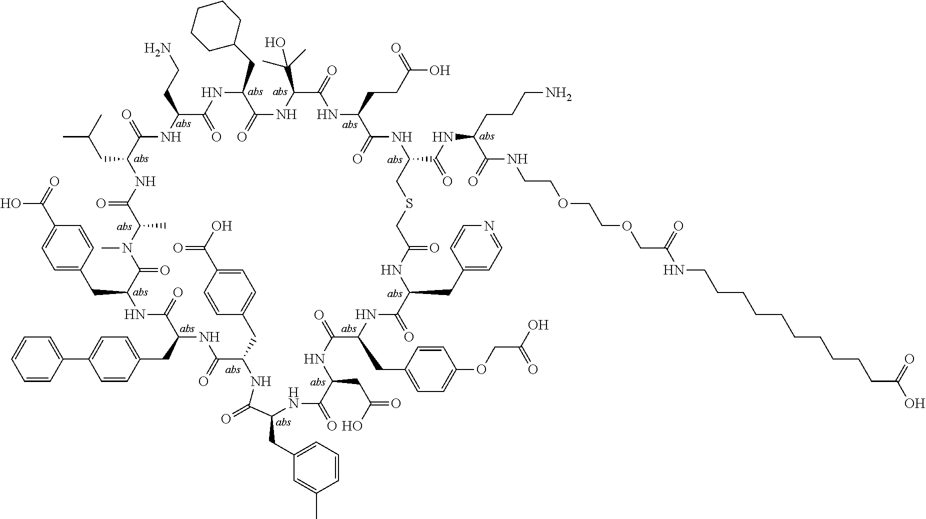 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2497 was prepared on a 50 μmol scale. The yield of the product was 13.9 mg, and its estimated purity by LCMS analysis was 98%. Analysis condition A: Retention time=1.52 min; ESI-MS(+) m/z [M+2H]²⁺: 1276.

### Preparation of Example 2498

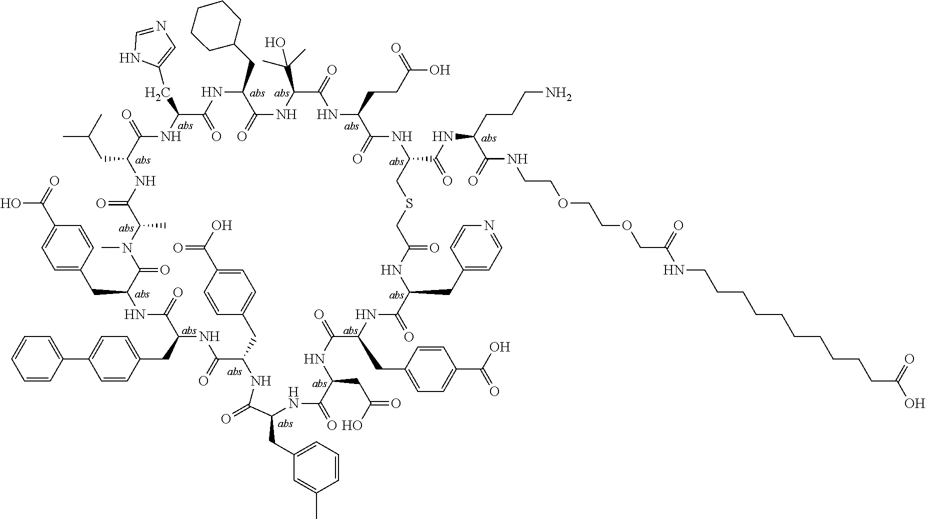 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2498 was prepared on a 50 μmol scale. The yield of the product was 66.6 mg, and its estimated purity by LCMS analysis was 100%. Analysis condition A: Retention time=1.36 min; ESI-MS(+) m/z [M+2H]²⁺: 1279.2.

### Preparation of Example 2499

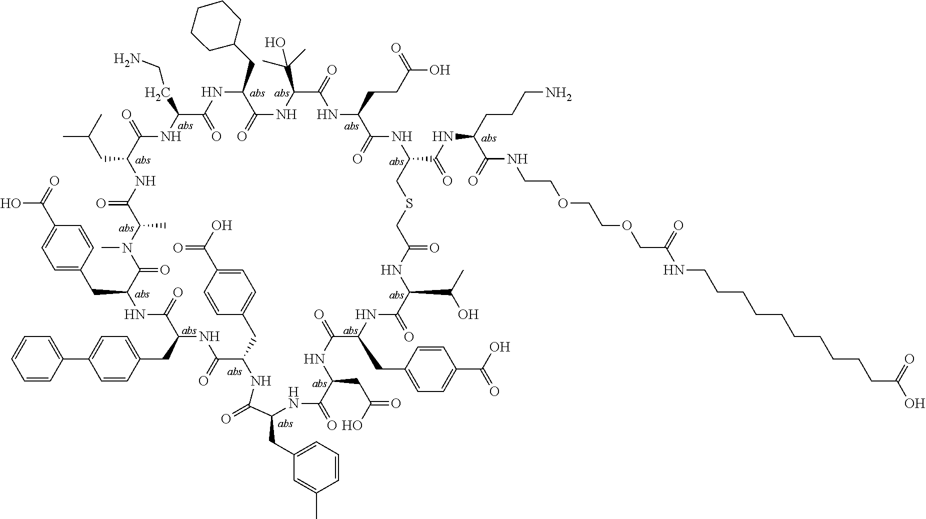 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2499 was prepared on a 50 μmol scale. The yield of the product was 73.5 mg, and its estimated purity by LCMS analysis was 95.1%. Analysis condition B: Retention time=1.86 min; ESI-MS(+) m/z [M+2H]²⁺: 1237.1.

### Preparation of Example 2500

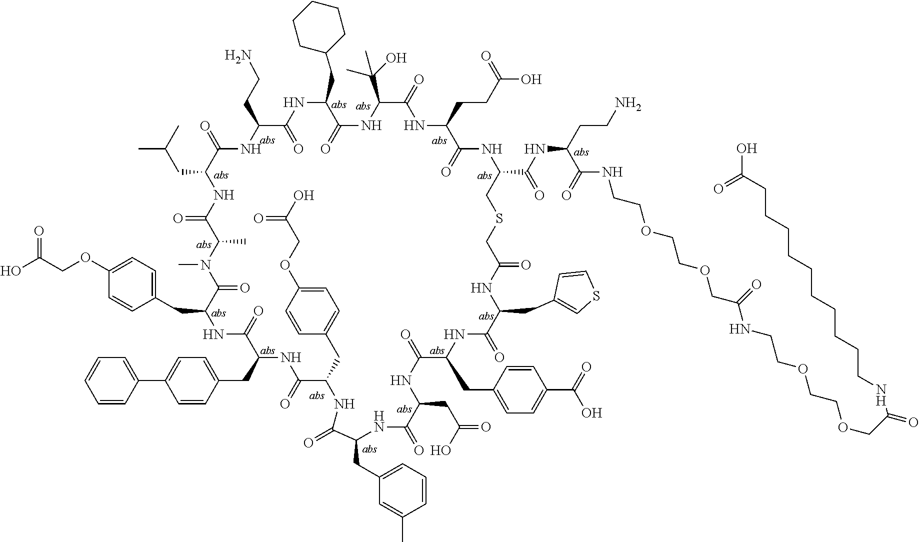 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2500 was prepared on a 30 μmol scale. The yield of the product was 24.2 mg, and its estimated purity by LCMS analysis was 97.3%. Analysis condition B: Retention time=1.9 min; ESI-MS(+) m/z [M+2H]²⁺: 1359.1.

### Preparation of Example 2501

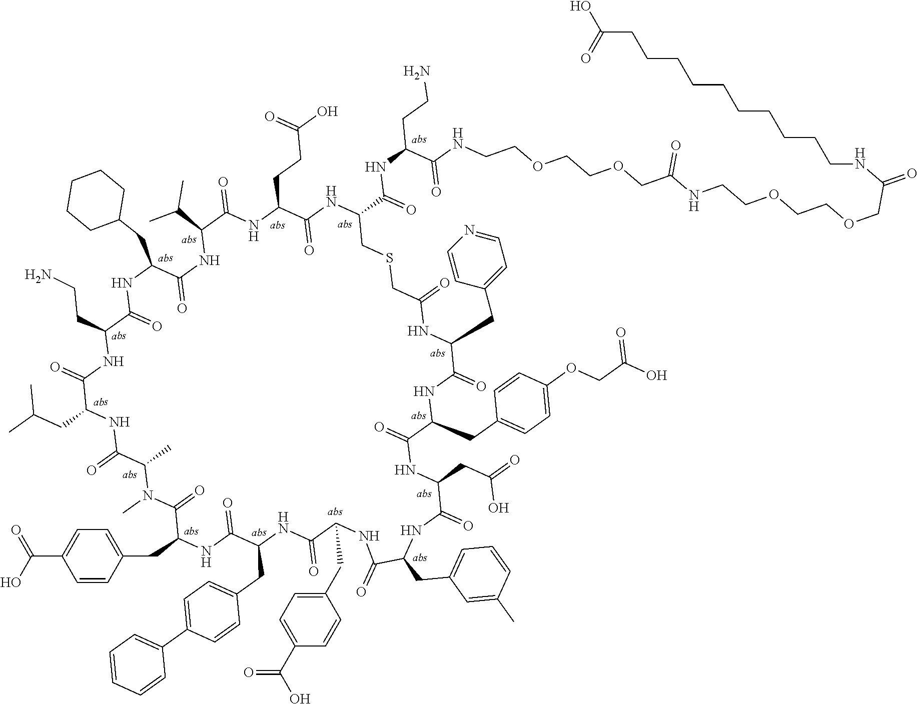 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2501 was prepared on a 30 μmol scale. The yield of the product was 8.5 mg, and its estimated purity by LCMS analysis was 90%. Analysis condition B: Retention time=1.75 min; ESI-MS(+) m/z [M+3H]³⁺: 889.2.

### Preparation of Example 2502

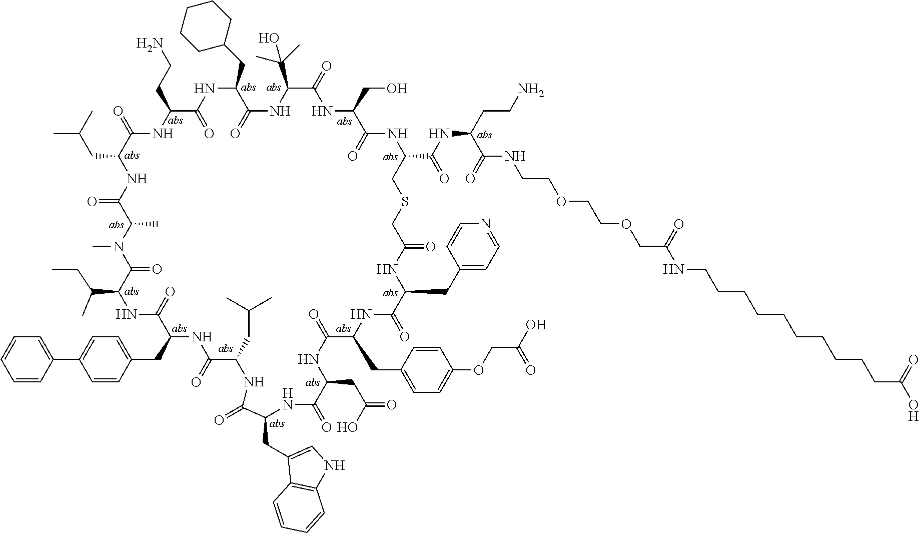 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2502 was prepared on a 50 μmol scale. The yield of the product was 11.7 mg, and its estimated purity by LCMS analysis was 91.4%. Analysis condition B: Retention time=1.79 min; ESI-MS(+) m/z [M+3H]³⁺: 788.7.

### Preparation of Example 2503

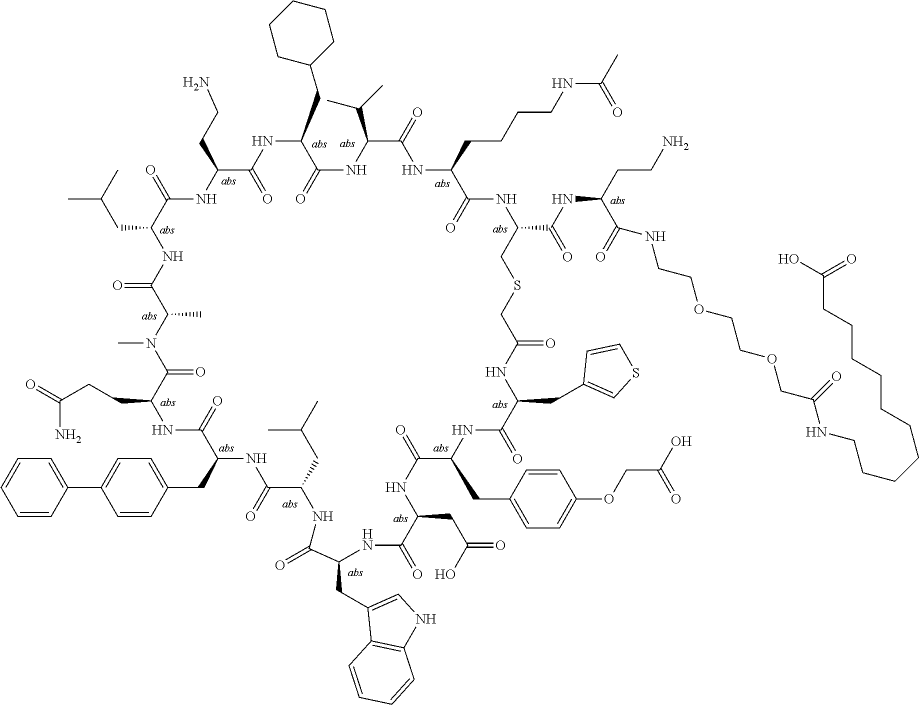 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCN)C(=O)NCCOCCOCC(=O)NCCCCCCCCCCC(=O)O)NC1=O]

Example 2503 was prepared on a 50 μmol scale. The yield of the product was 50.3 mg, and its estimated purity by LCMS analysis was 95.3%. Analysis condition B: Retention time=1.84 min; ESI-MS(+) m/z [M+2H]²⁺: 1226.2.

### Preparation of Example 2504

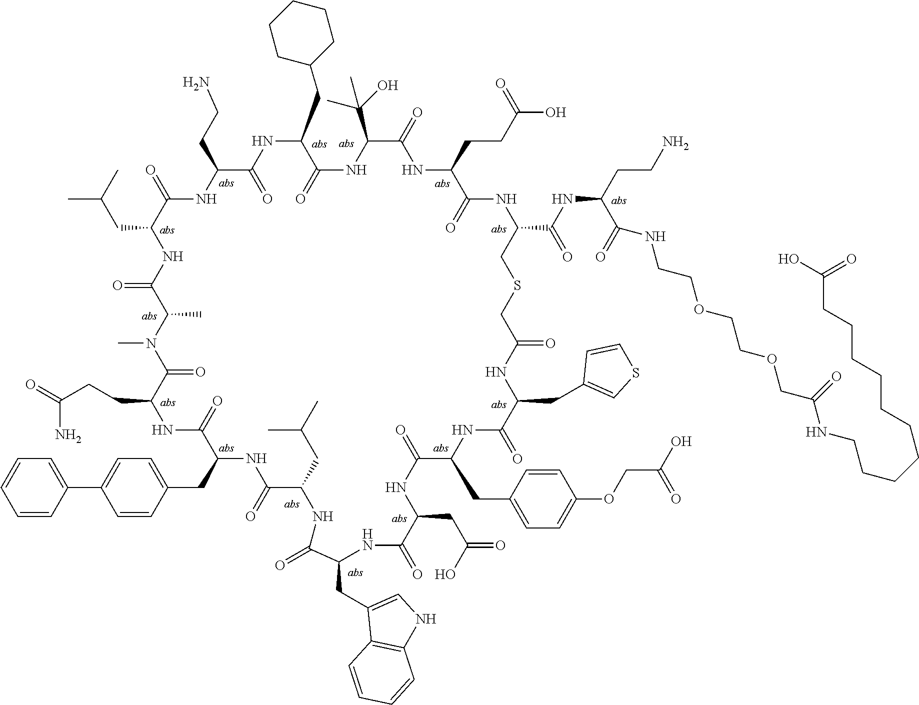 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2504 was prepared on a 50 μmol scale. The yield of the product was 99.6 mg, and its estimated purity by LCMS analysis was 91.4%. Analysis condition B: Retention time=1.99 min; ESI-MS(+) m/z [M+2H]²⁺: 1213.1.

### Preparation of Example 2505

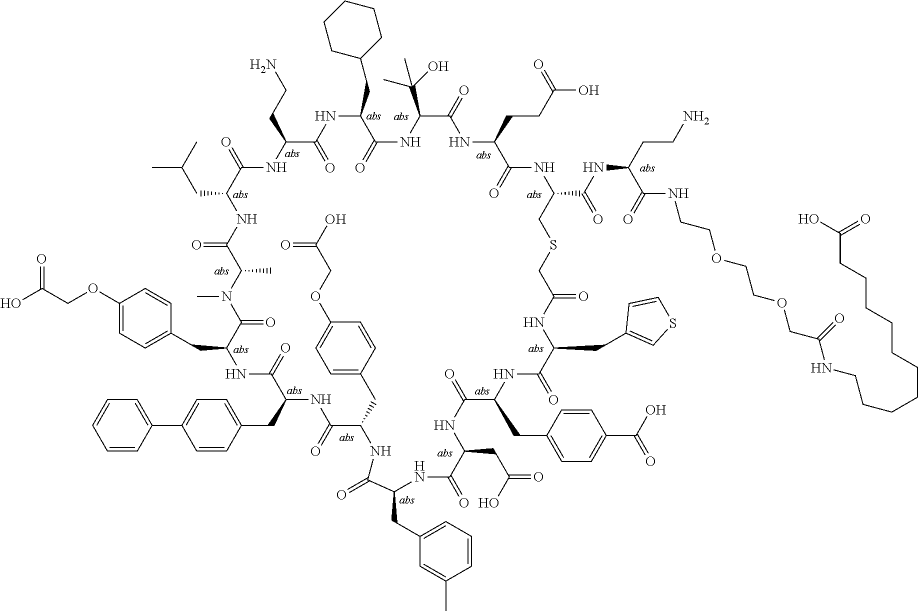 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2505 was prepared on a 50 μmol scale. The yield of the product was 88.1 mg, and its estimated purity by LCMS analysis was 95%. Analysis condition A: Retention time=1.56 min; ESI-MS(+) m/z [M+2H]²⁺: 1287.1.

### Preparation of Example 2506

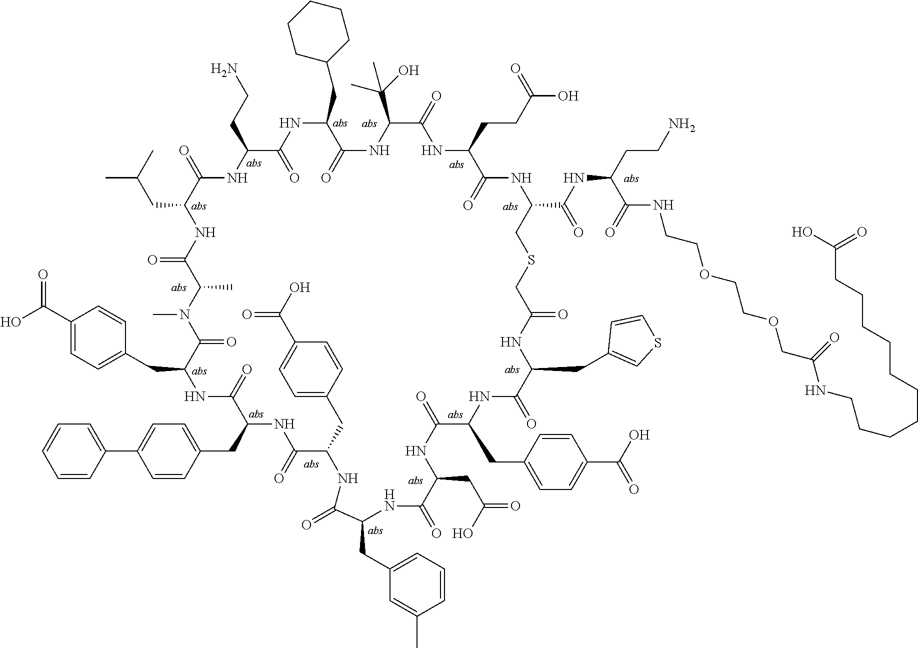 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2506 was prepared on a 50 μmol scale. The yield of the product was 44.2 mg, and its estimated purity by LCMS analysis was 97.2%. Analysis condition B: Retention time=1.88 min; ESI-MS(+) m/z [M+2H]²⁺: 1256.

### Preparation of Example 2507

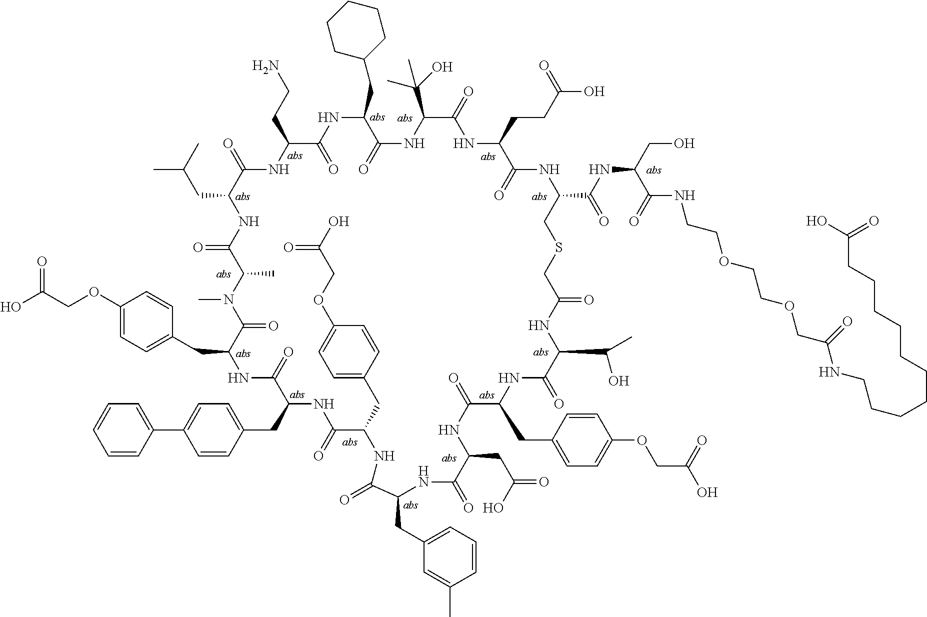 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CO)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2507 was prepared on a 50 μmol scale. The yield of the product was 42.4 mg, and its estimated purity by LCMS analysis was 100%. Analysis condition A: Retention time=1.37 min; ESI-MS(+) m/z [M+2H]²⁺: 1268.4.

### Preparation of Example 2508

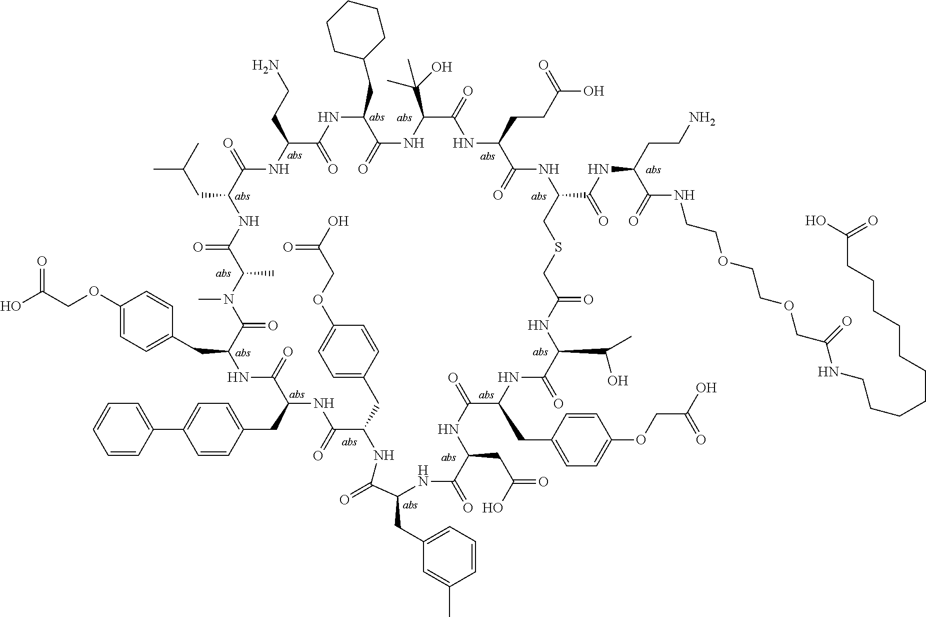 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2508 was prepared on a 40 μmol scale. The yield of the product was 20 mg, and its estimated purity by LCMS analysis was 81.9%. Analysis condition A: Retention time=1.43 min; ESI-MS(+) m/z [M+2H]²⁺: 1275.9.

### Preparation of Example 2509

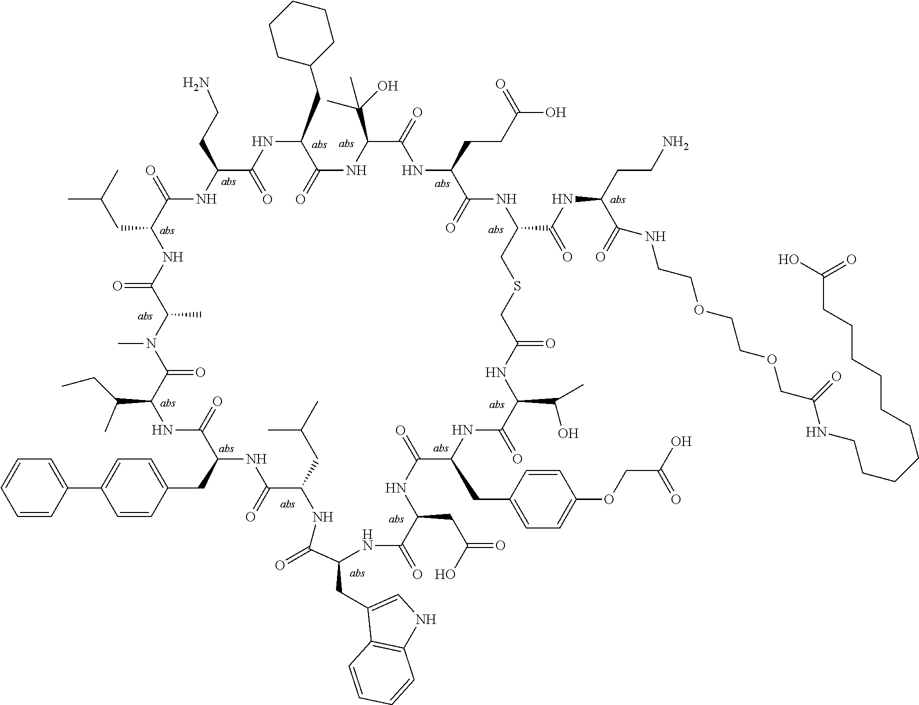 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2509 was prepared on a 40 μmol scale. The yield of the product was 41.9 mg, and its estimated purity by LCMS analysis was 90%. Analysis condition B: Retention time=1.9 min; ESI-MS(+) m/z [M+2H]²⁺: 1180.3.

### Preparation of Example 2510

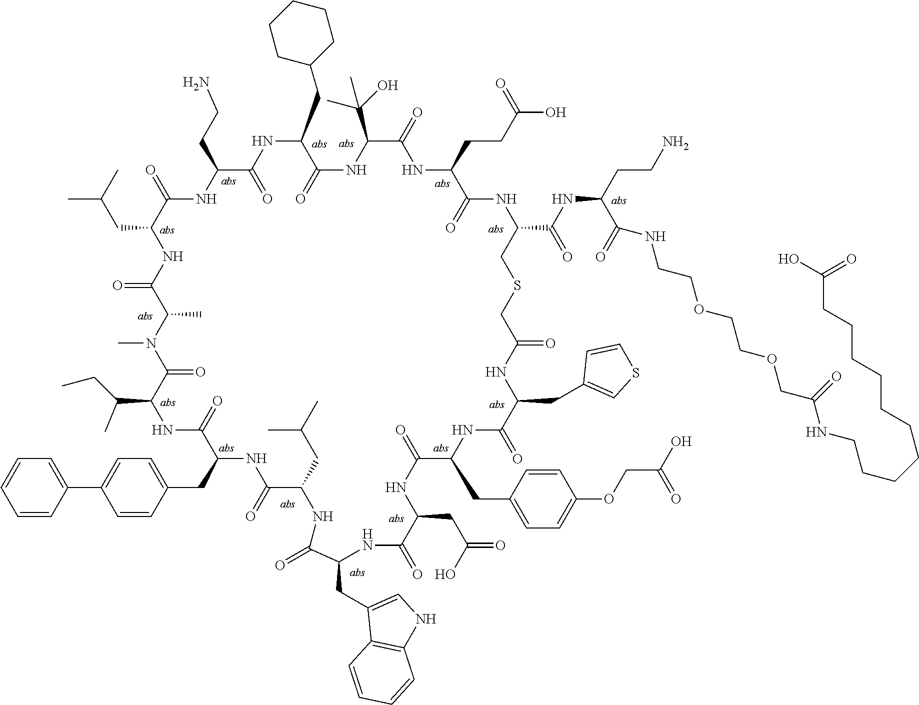 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2510 was prepared on a 40 μmol scale. The yield of the product was 29.6 mg, and its estimated purity by LCMS analysis was 96.3%. Analysis condition B: Retention time=1.91 min; ESI-MS(+) m/z [M+2H]²⁺: 1206.2.

### Preparation of Example 2511

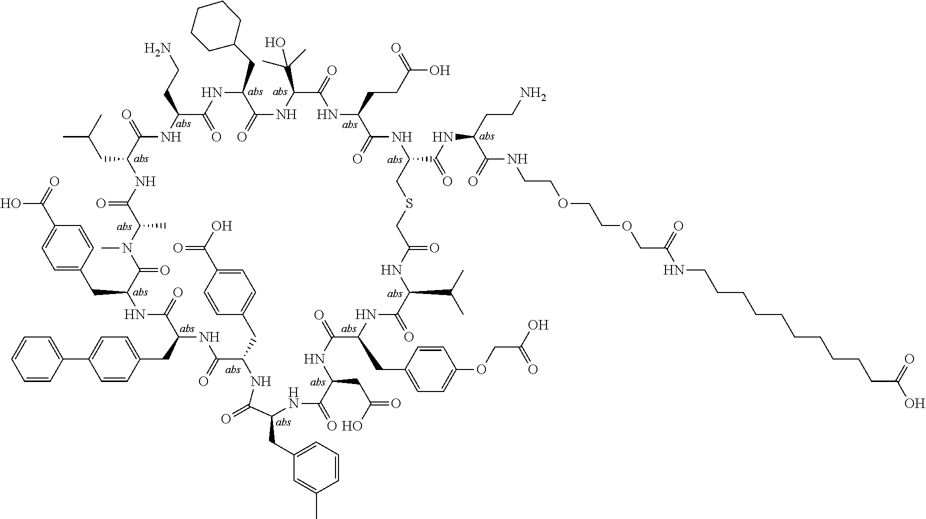 [SMILES: Cc1cccc(C[C@@H]2NC(=O)[C@H](CC(=O)O)NC(=O)[C@H](Cc3ccc(OCC(=O)O)cc3)NC(=O)[C@H](C(C)C)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2511 was prepared on a 40 μmol scale. The yield of the product was 75 mg, and its estimated purity by LCMS analysis was 97.7%. Analysis condition A: Retention time=1.43 min; ESI-MS(+) m/z [M+2H]²⁺: 1245.1.

### Preparation of Example 2512

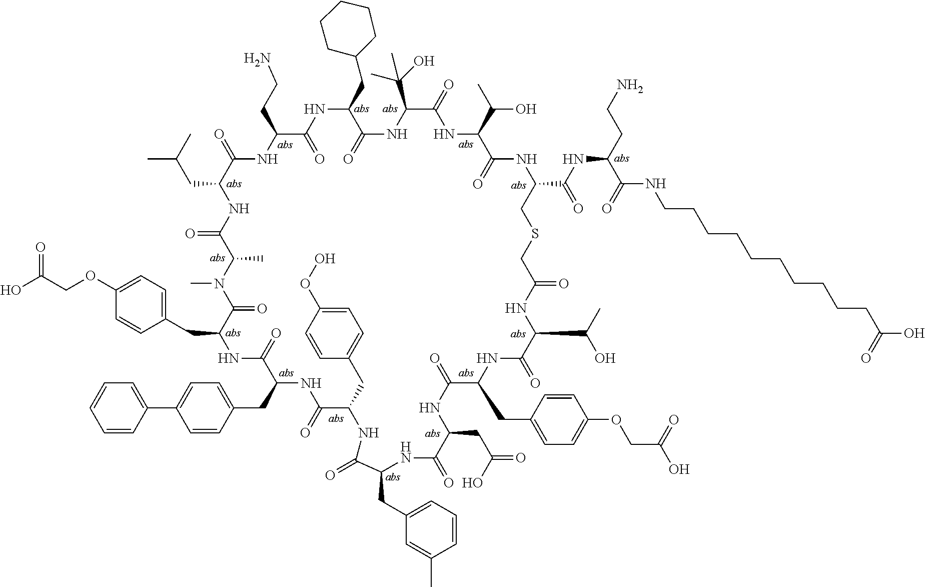

Example 2512 was prepared on a 50 μmol scale. The yield of the product was 78.2 mg, and its estimated purity by LCMS analysis was 100%. Analysis condition A: Retention time=1.51 min; ESI-MS(+) m/z [M+2H]²⁺: 1174.

### Preparation of Example 2513

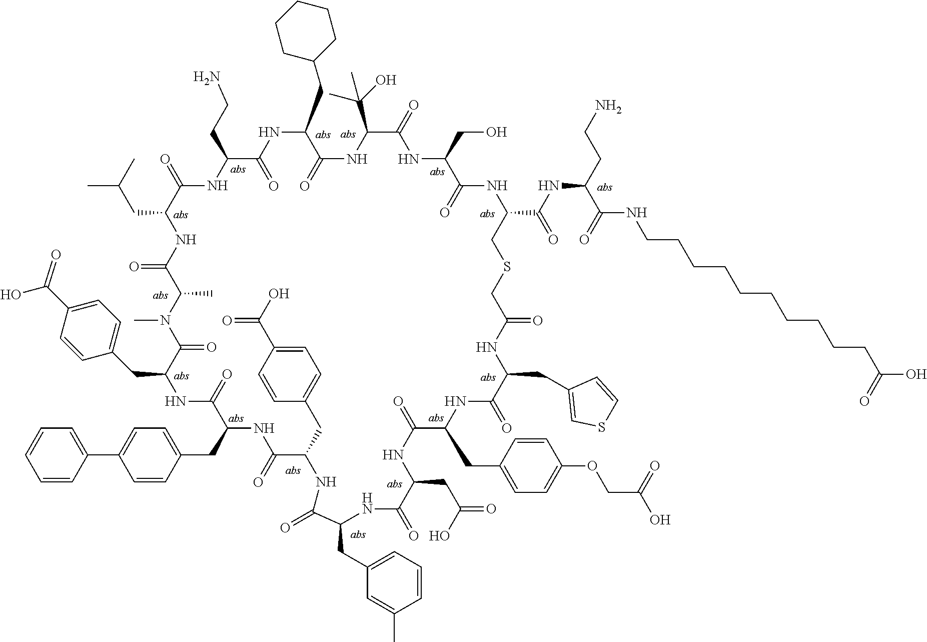 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccsc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2513 was prepared on a 50 μmol scale. The yield of the product was 19.6 mg, and its estimated purity by LCMS analysis was 98.9%. Analysis condition B: Retention time=1.92 min; ESI-MS(+) m/z [M+2H]²⁺: 1178.1.

### Preparation of Example 2514

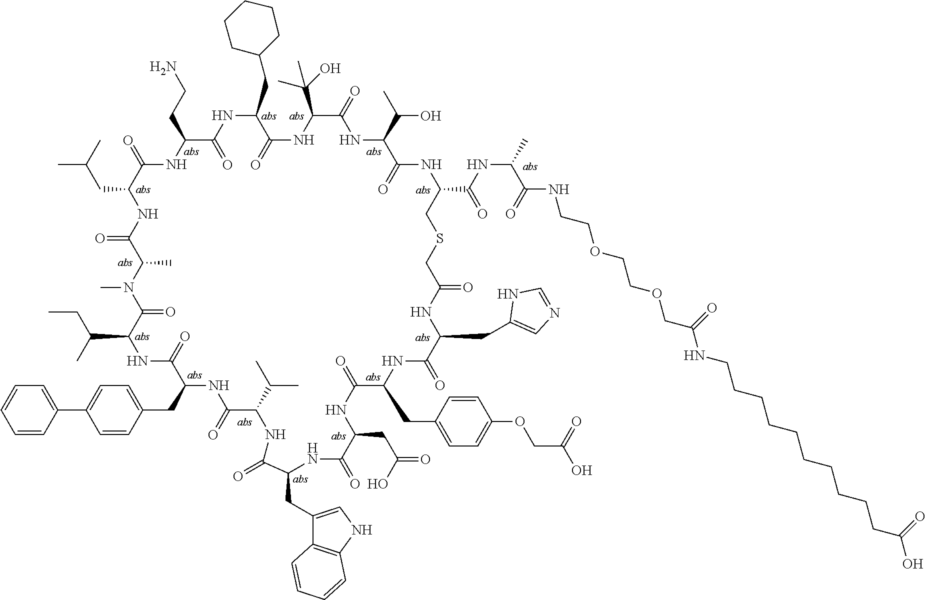 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@H](C)C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2514 was prepared on a 50 μmol scale. The yield of the product was 65.4 mg, and its estimated purity by LCMS analysis was 96.4%. Analysis condition A: Retention time=1.73 min; ESI-MS(+) m/z [M+2H]²⁺: 1162.

### Preparation of Example 2515

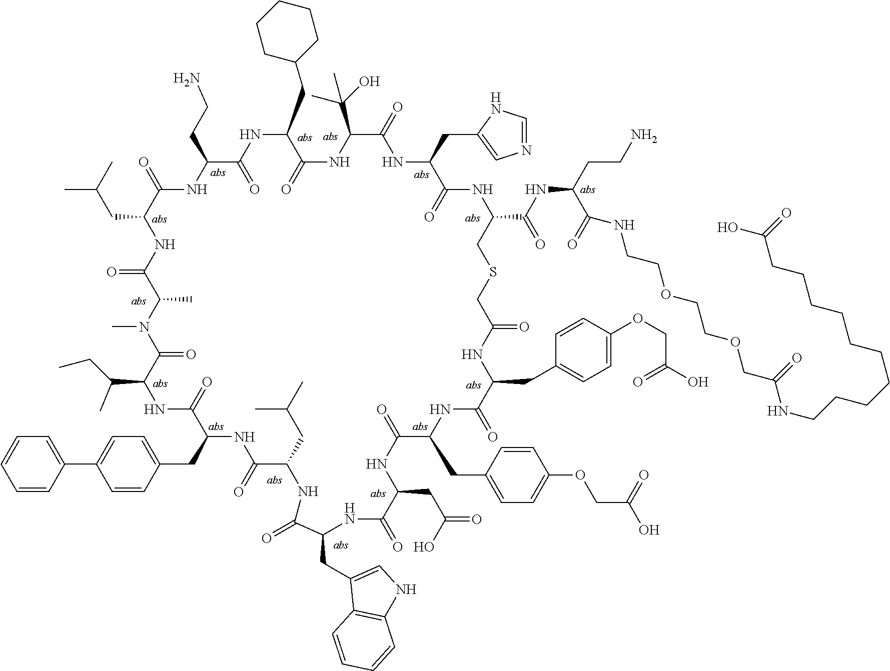 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2515 was prepared on a 50 μmol scale. The yield of the product was 11.6 mg, and its estimated purity by LCMS analysis was 97.8%. Analysis condition A: Retention time=1.65 min; ESI-MS(+) m/z [M+2H]²⁺: 1244.2.

### Preparation of Example 2516

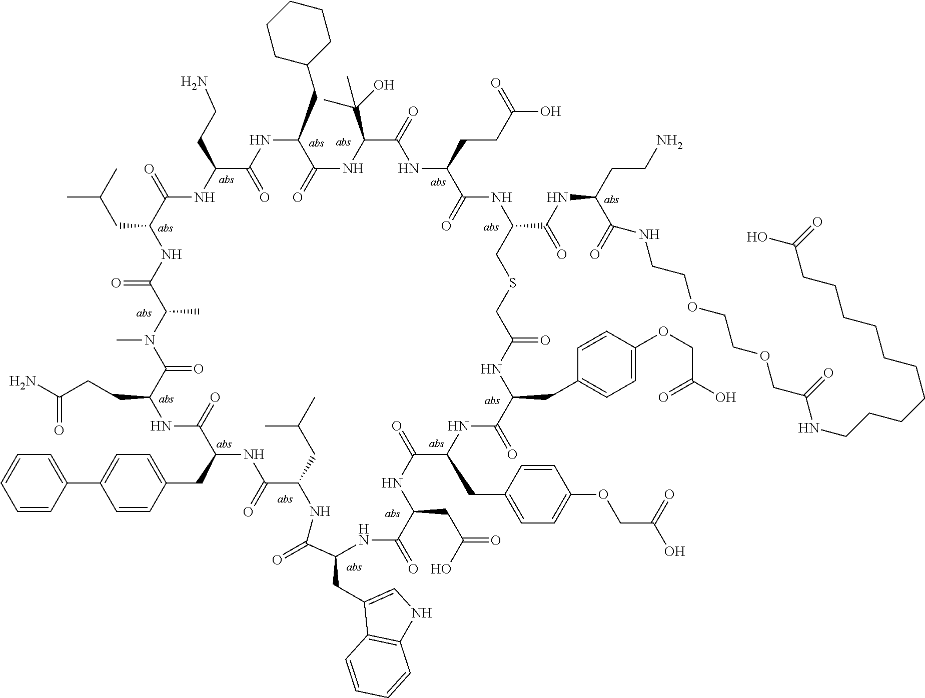 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2516 was prepared on a 50 μmol scale. The yield of the product was 61.4 mg, and its estimated purity by LCMS analysis was 98.8%. Analysis condition A: Retention time=1.67 min; ESI-MS(+) m/z [M+2H]²⁺: 1247.2.

### Preparation of Example 2517

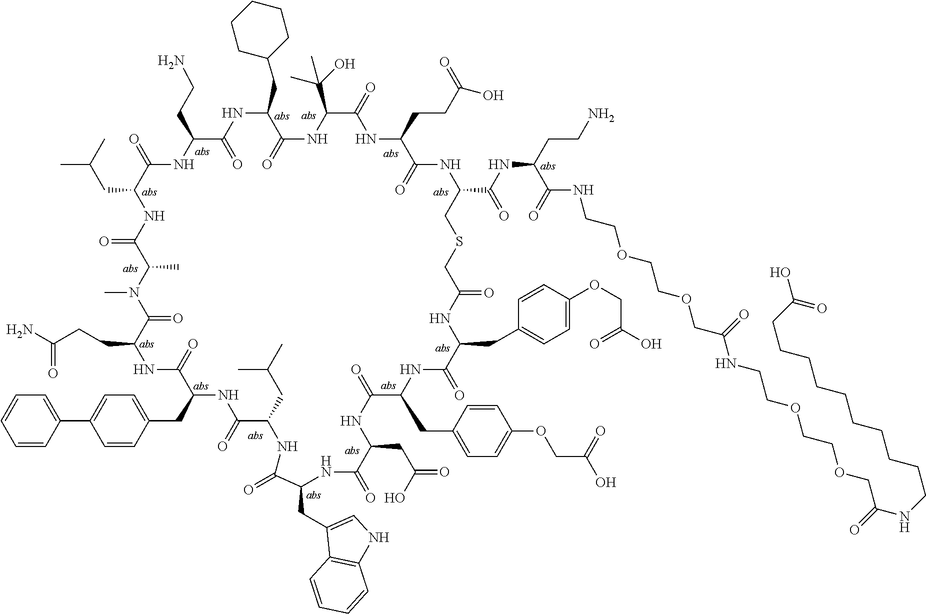 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC1=O]

Example 2517 was prepared on a 50 μmol scale. The yield of the product was 54.1 mg, and its estimated purity by LCMS analysis was 96.1%. Analysis condition A: Retention time=1.51 min; ESI-MS(+) m/z [M+2H]²⁺: 1320.2.

### Preparation of Example 2518

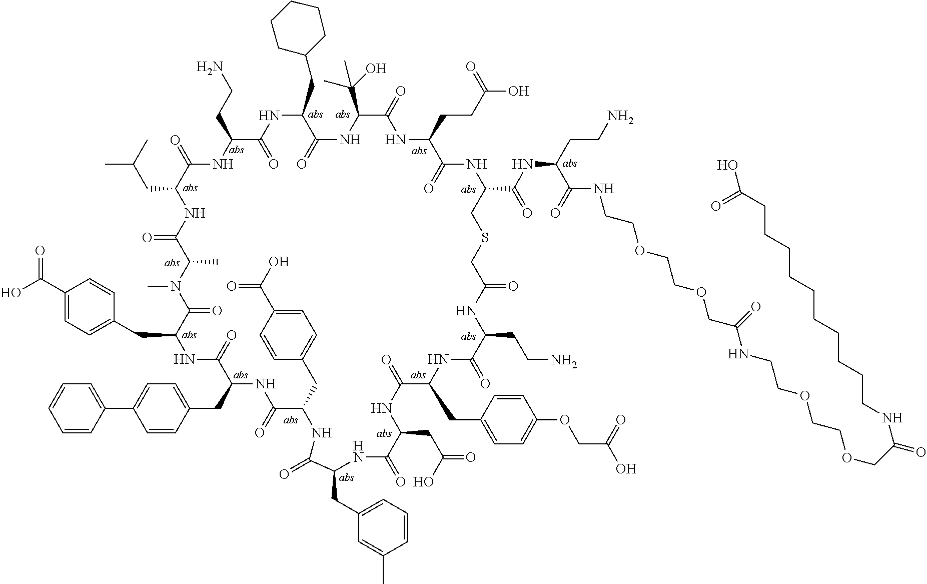 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2518 was prepared on a 50 μmol scale. The yield of the product was 128 mg, and its estimated purity by LCMS analysis was 97.7%. Analysis condition A: Retention time=1.44, 1.48 min; ESI-MS(+) m/z [M+2H]²⁺: 879.

### Preparation of Example 2519

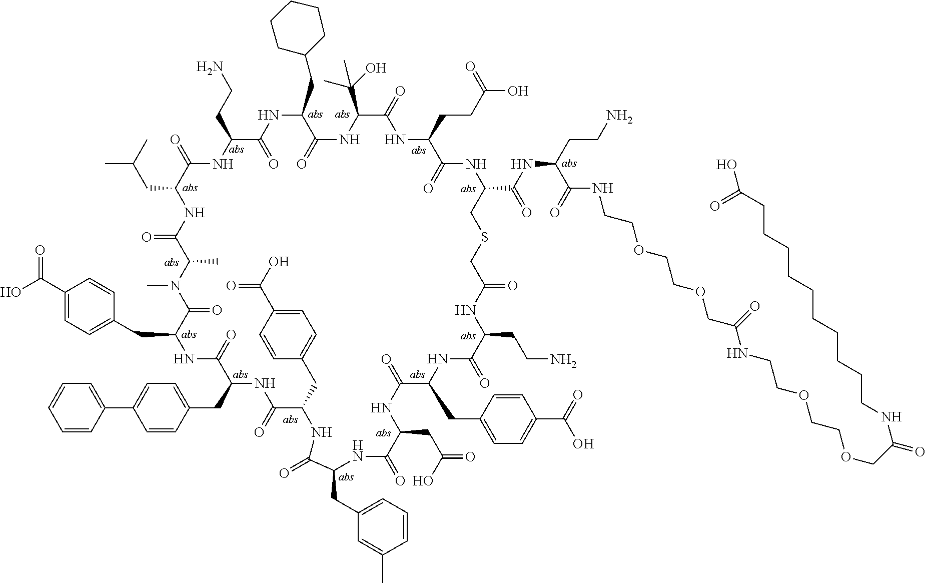 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2519 was prepared on a 50 μmol scale. The yield of the product was 37.8 mg, and its estimated purity by LCMS analysis was 96%. Analysis condition B: Retention time=1.8 min; ESI-MS(+) m/z [M+3H]³⁺: 868.9.

### Preparation of Example 2520

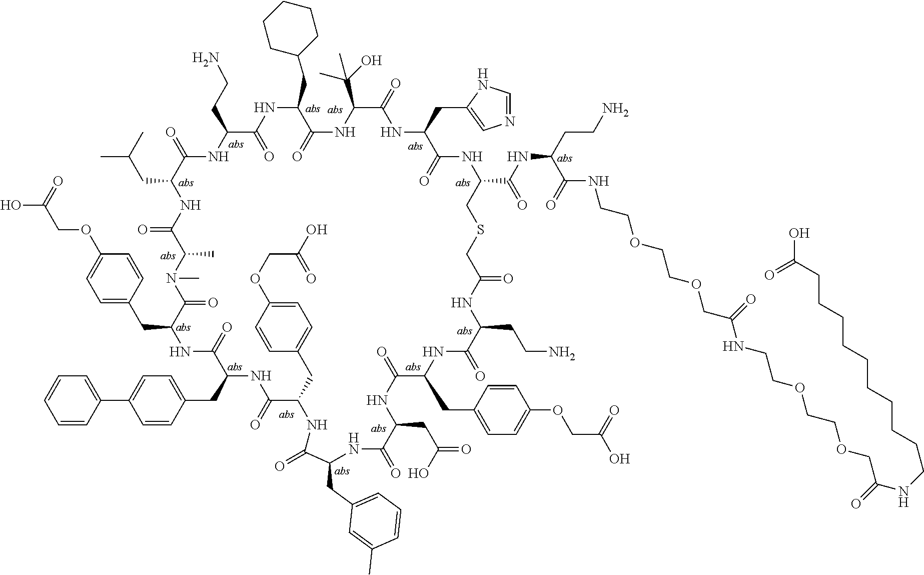 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2520 was prepared on a 50 μmol scale. The yield of the product was 24 mg, and its estimated purity by LCMS analysis was 97%. Analysis condition A: Retention time=1.64 min; ESI-MS(+) m/z [M+2H]²⁺: 901.1.

### Preparation of Example 2521

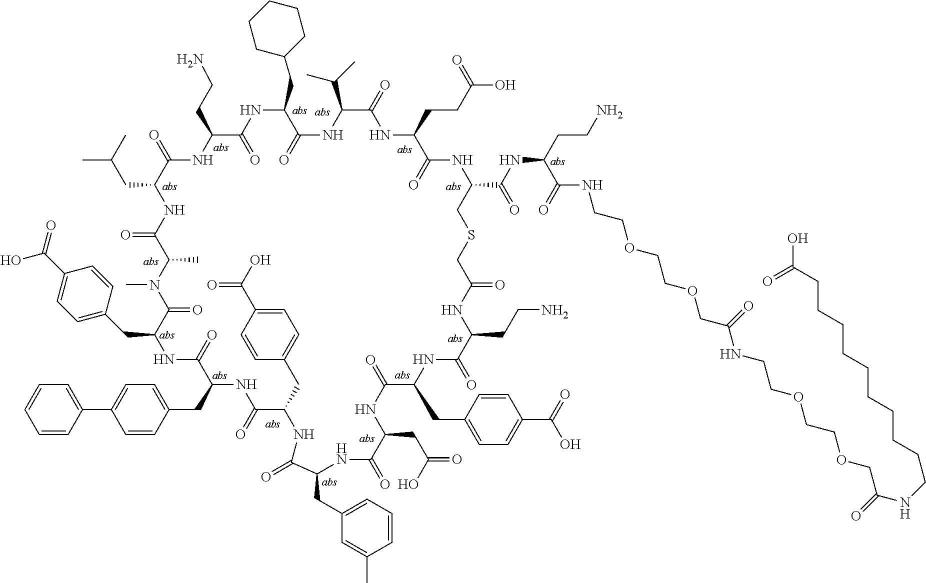 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2521 was prepared on a 50 μmol scale. The yield of the product was 19.7 mg, and its estimated purity by LCMS analysis was 96.4%. Analysis condition A: Retention time=1.48 min; ESI-MS(+) m/z [M+2H]²⁺: 1294.1.

### Preparation of Example 2522

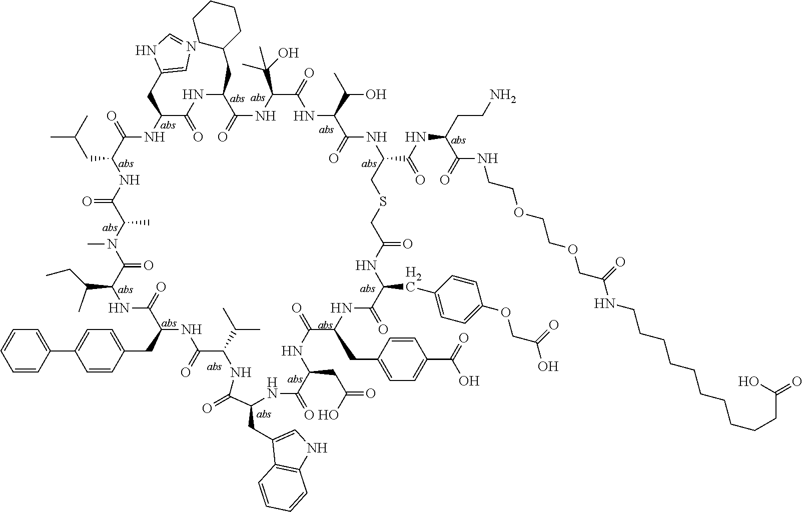 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](Cc2cnc[nH]2)NC(=O)[C@@H](CC(C)C)NC(=O)[C@H](C)N(C)C1=O]

Example 2522 was prepared on a 50 μmol scale. The yield of the product was 14.2 mg, and its estimated purity by LCMS analysis was 98.7%. Analysis condition A: Retention time=1.57 min; ESI-MS(+) m/z [M+2H]²⁺: 1223.3.

### Preparation of Example 2523

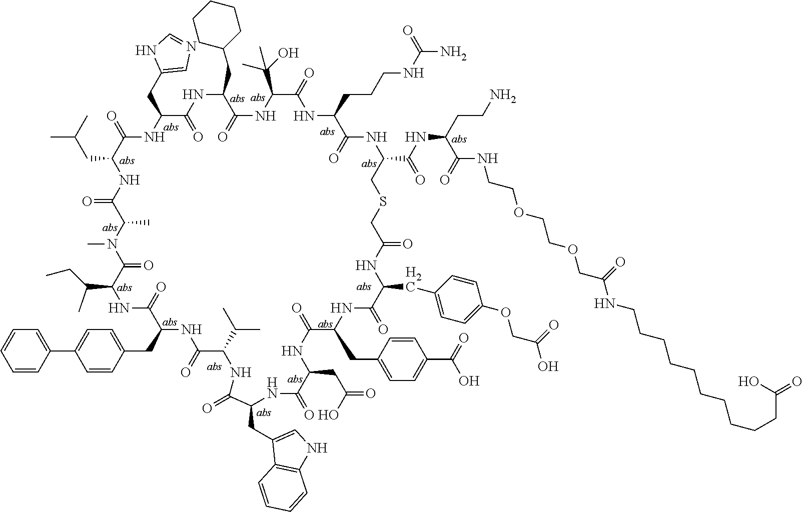 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCNC(N)=O)NC(=O)[C@H](C(C)(C)O)NC(=O)[C@H](CC2CCCCC2)NC(=O)[C@H](Cc2cnc[nH]2)NC(=O)[C@@H](CC(C)C)NC(=O)[C@H](C)N(C)C1=O]

Example 2523 was prepared on a 50 μmol scale. The yield of the product was 25.2 mg, and its estimated purity by LCMS analysis was 100%. Analysis condition A: Retention time=1.73 min; ESI-MS(+) m/z [M+2H]²⁺: 1250.9.

### Preparation of Example 2524

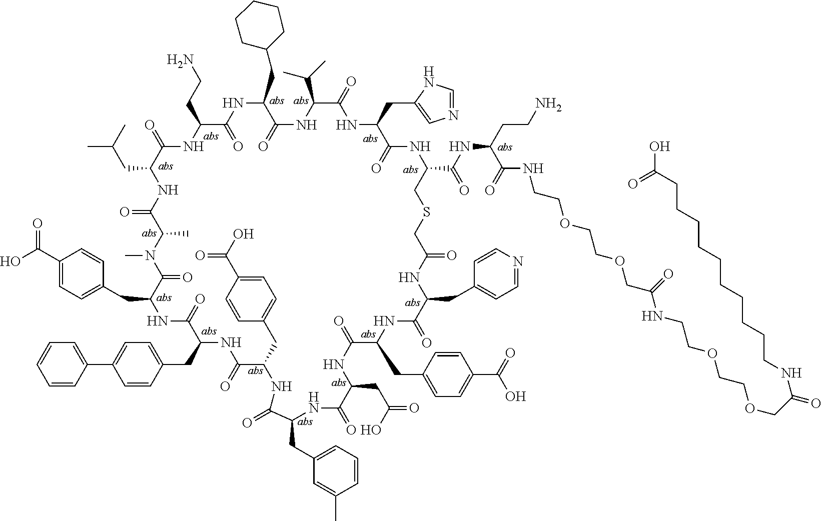 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2524 was prepared on a 50 μmol scale. The yield of the product was 28.5 mg, and its estimated purity by LCMS analysis was 96.3%. Analysis condition A: Retention time=1.47 min; ESI-MS(+) m/z [M+3H]³⁺: 882.3.

### Preparation of Example 2525

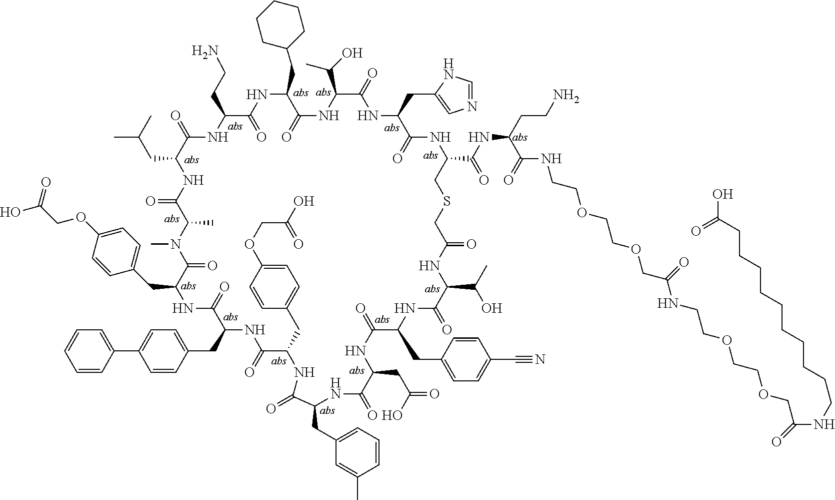 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2525 was prepared on a 50 μmol scale. The yield of the product was 54.6 mg, and its estimated purity by LCMS analysis was 100%. Analysis condition A: Retention time=1.8 min; ESI-MS(+) m/z [M+2H]²⁺: 1321.

### Preparation of Example 2526

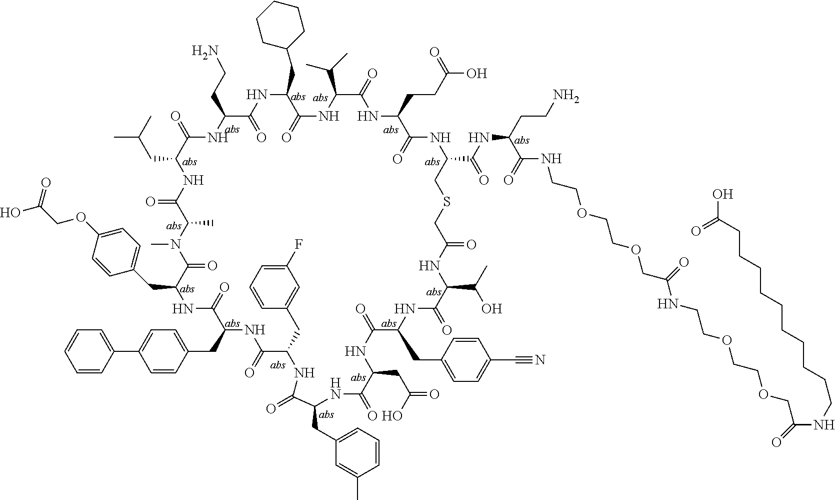 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Example 2526 was prepared on a 50 μmol scale. The yield of the product was 31 mg, and its estimated purity by LCMS analysis was 95.6%. Analysis condition A: Retention time=1.85 min; ESI-MS(+) m/z [M+2H]²⁺: 1287.

### Preparation of Example 2527

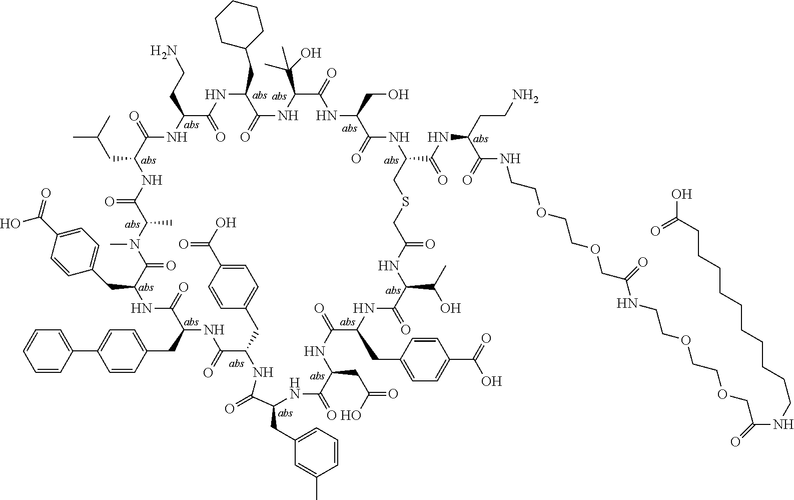 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2527 was prepared on a 50 μmol scale. The yield of the product was 25.4 mg, and its estimated purity by LCMS analysis was 91.6%. Analysis condition B: Retention time=1.79 min; ESI-MS(+) m/z [M+2H]²⁺: 1282.2.

### Preparation of Example 2528

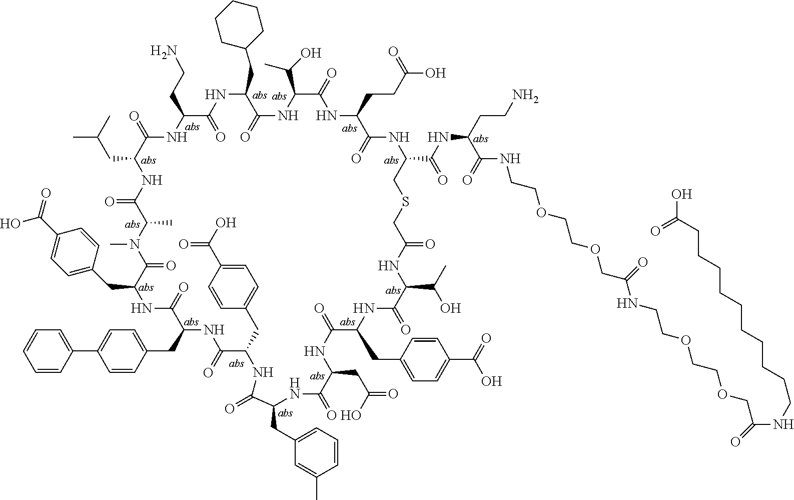 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2528 was prepared on a 50 μmol scale. The yield of the product was 49.3 mg, and its estimated purity by LCMS analysis was 97.5%. Analysis condition B: Retention time=1.92 min; ESI-MS(+) m/z [M+2H]²⁺: 1296.

### Preparation of Example 2529

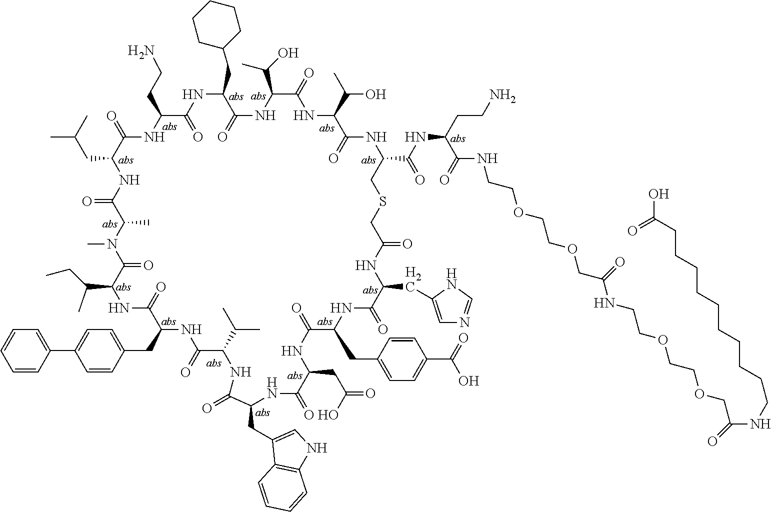 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](C(C)O)NC(=O)[C@H](C(C)O)NC(=O)[C@H](CC2CCCCC2)NC(=O)[C@H](CCN)NC(=O)[C@@H](CC(C)C)NC(=O)[C@H](C)N(C)C1=O]

Example 2529 was prepared on a 50 μmol scale. The yield of the product was 29.2 mg, and its estimated purity by LCMS analysis was 95.8%. Analysis condition A: Retention time=1.68 min; ESI-MS(+) m/z [M+3H]³⁺: 819.1.

### Preparation of Example 2530

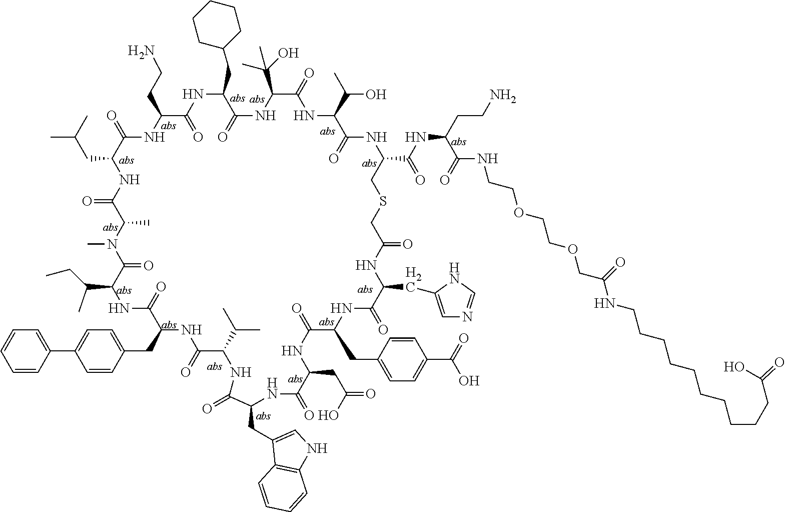 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2530 was prepared on a 50 μmol scale. The yield of the product was 33.7 mg, and its estimated purity by LCMS analysis was 98.2%. Analysis condition A: Retention time=1.67 min; ESI-MS(+) m/z [M+2H]²⁺: 1162.3.

### Preparation of Example 2531

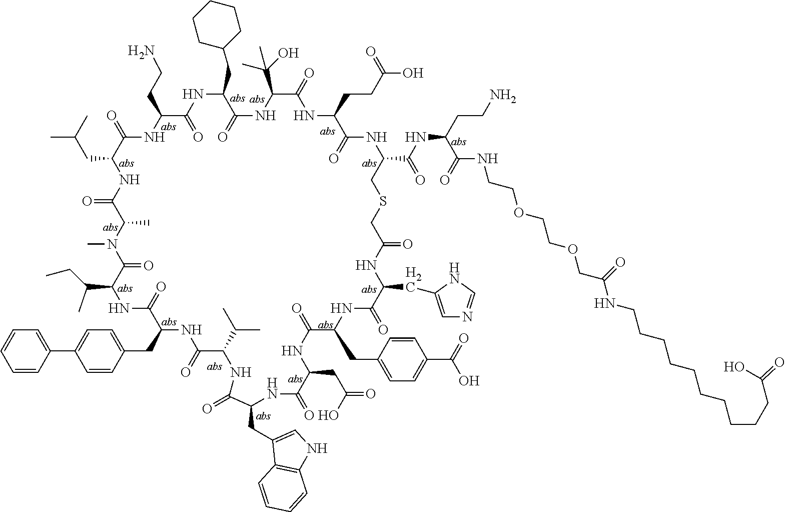 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2531 was prepared on a 50 μmol scale. The yield of the product was 42.8 mg, and its estimated purity by LCMS analysis was 96.9%. Analysis condition A: Retention time=1.63 min; ESI-MS(+) m/z [M+2H]²⁺: 1176.3.

### Preparation of Example 2532

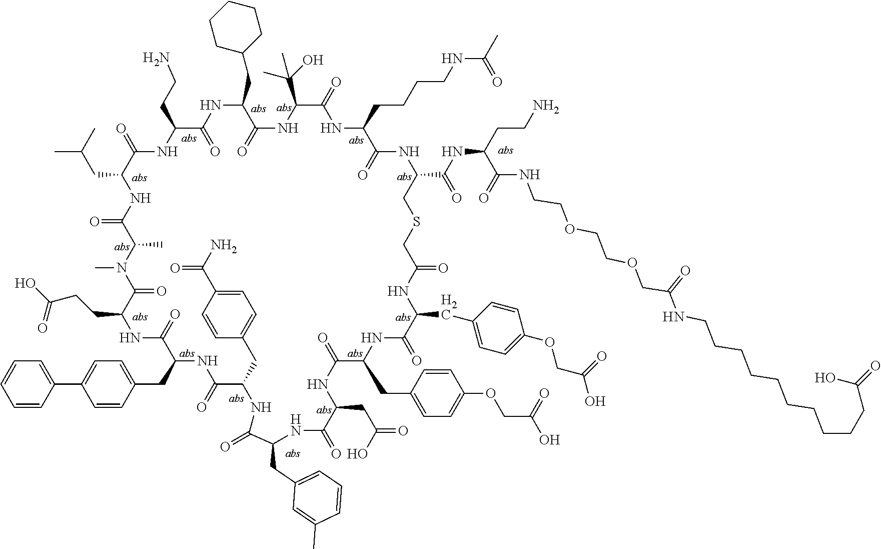 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(=O)O)NC(=O)[C@H](Cc2ccc(-c3ccccc3)cc2)NC(=O)[C@H](Cc2ccc(C(N)=O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2532 was prepared on a 50 μmol scale. The yield of the product was 53.1 mg, and its estimated purity by LCMS analysis was 95.8%. Analysis condition A: Retention time=1.53 min; ESI-MS(+) m/z [M+2H]²⁺: 1294.5.

### Preparation of Example 2533

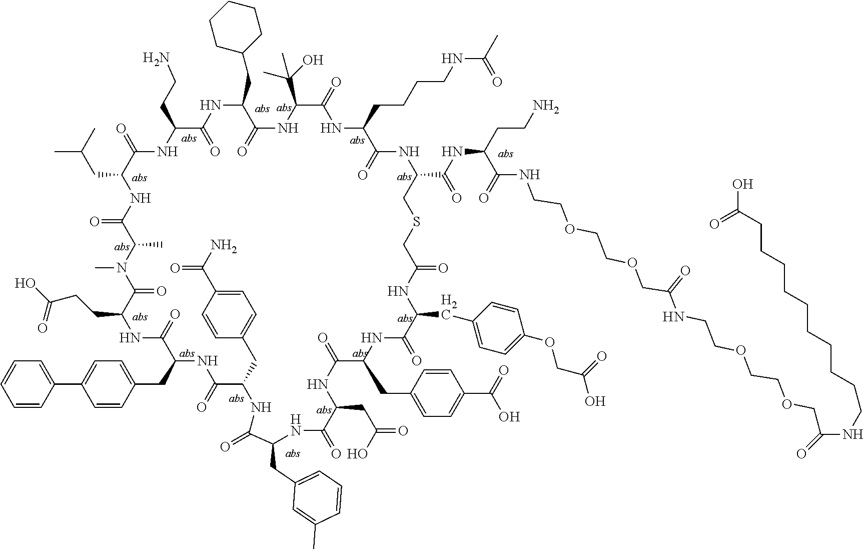 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(=O)O)NC(=O)[C@H](Cc2ccc(-c3ccccc3)cc2)NC(=O)[C@H](Cc2ccc(C(N)=O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCC(=O)O)NC1=O]

Example 2533 was prepared on a 50 μmol scale. The yield of the product was 58.3 mg, and its estimated purity by LCMS analysis was 96%. Analysis condition A: Retention time=1.6, 1.64 min; ESI-MS(+) m/z [M+2H]²⁺: 1352.

### Preparation of Example 2534

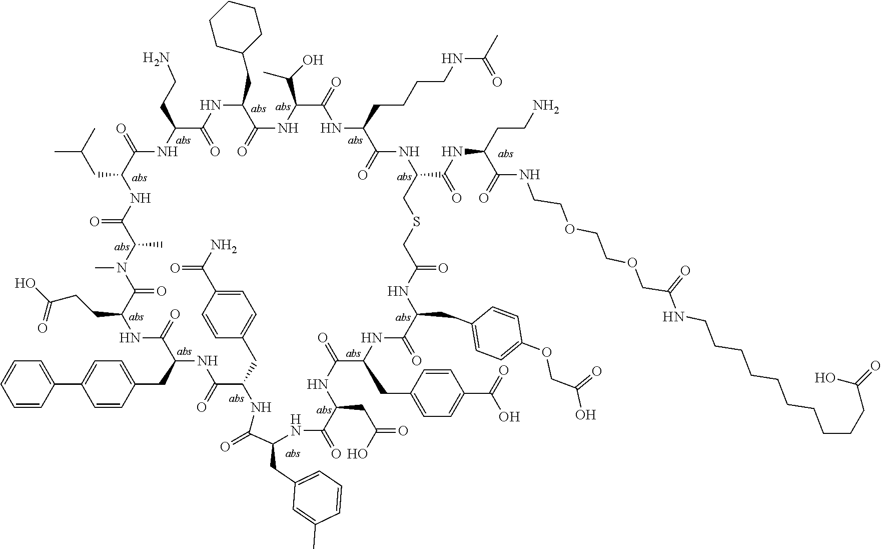 [SMILES: CC(=O)NCCCC[C@@H]1NC(=O)[C@H](C(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(=O)O)NC(=O)[C@H](Cc2ccc(-c3ccccc3)cc2)NC(=O)[C@H](Cc2ccc(C(N)=O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2534 was prepared on a 50 μmol scale. The yield of the product was 18.6 mg, and its estimated purity by LCMS analysis was 100%. Analysis condition A: Retention time=1.59 min; ESI-MS(+) m/z [M+2H]²⁺: 1273.

### Preparation of Example 2535

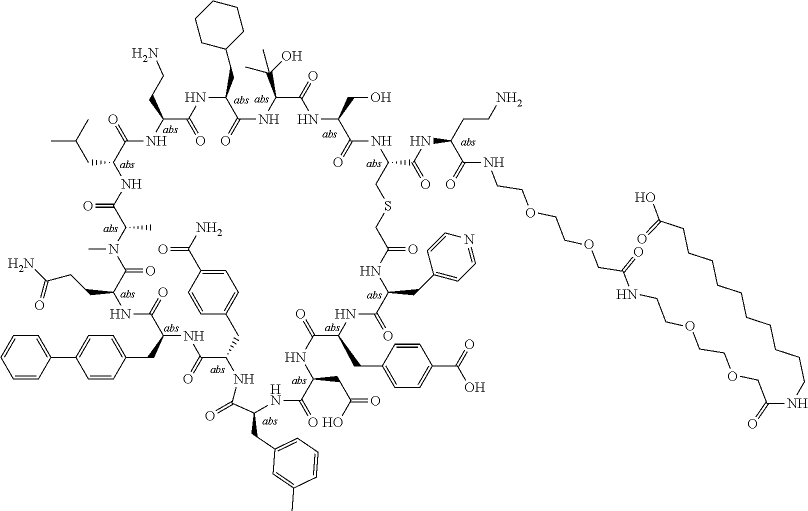 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](Cc3ccc(C(N)=O)cc3)NC2=O)c1]

Example 2535 was prepared on a 50 μmol scale. The yield of the product was 6.2 mg, and its estimated purity by LCMS analysis was 100%. Analysis condition A: Retention time=1.68 min; ESI-MS(+) m/z [M+2H]²⁺: 1273.9.

### Preparation of Example 2536

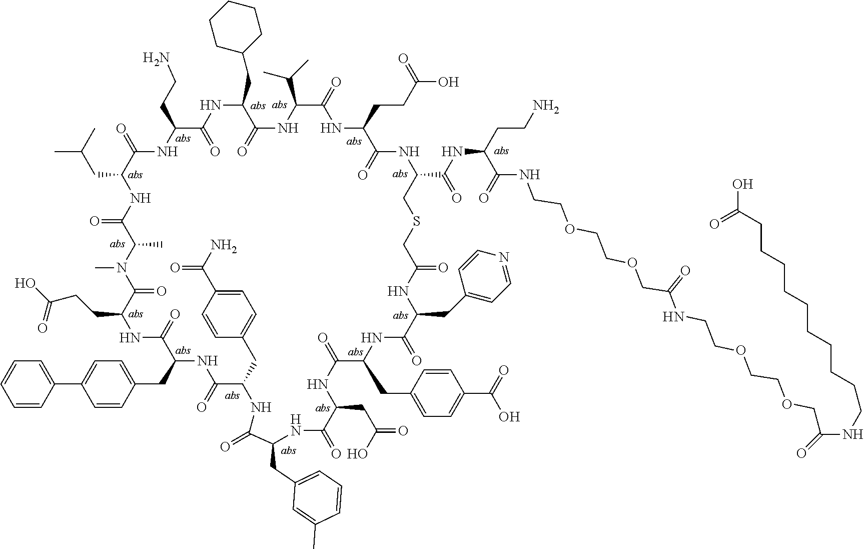 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCC(=O)O)NC(=O)[C@H](Cc3ccc(-c4ccccc4)cc3)NC(=O)[C@H](Cc3ccc(C(N)=O)cc3)NC2=O)c1]

Example 2536 was prepared on a 50 μmol scale. The yield of the product was 10.3 mg, and its estimated purity by LCMS analysis was 95.6% a. Analysis condition B: Retention time=1.68 min; ESI-MS(+) m/z [M+3H]³⁺: 858.2.

### Preparation of Example 2537

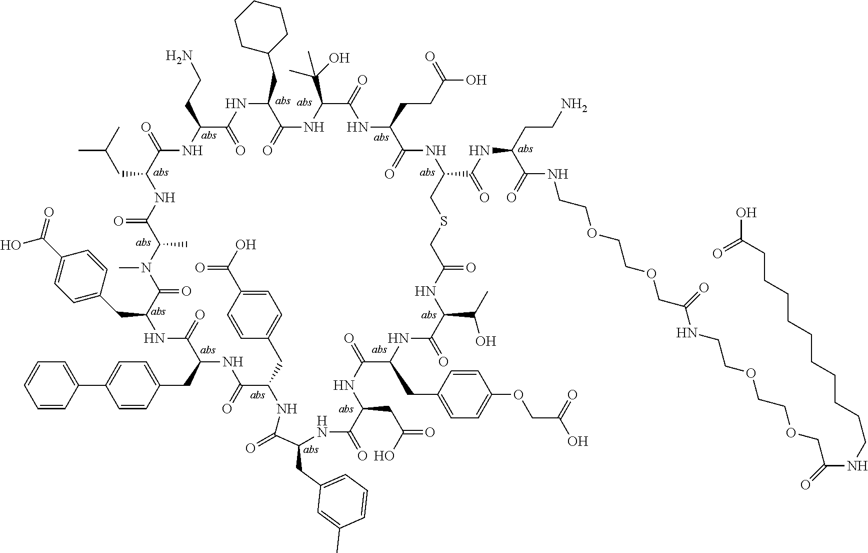 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2537 was prepared on a 50 μmol scale. The yield of the product was 88.6 mg, and its estimated purity by LCMS analysis was 96.3%. Analysis condition A: Retention time=1.42 min; ESI-MS(+) m/z [M+2H]²⁺: 1318.2.

### Preparation of Example 2538

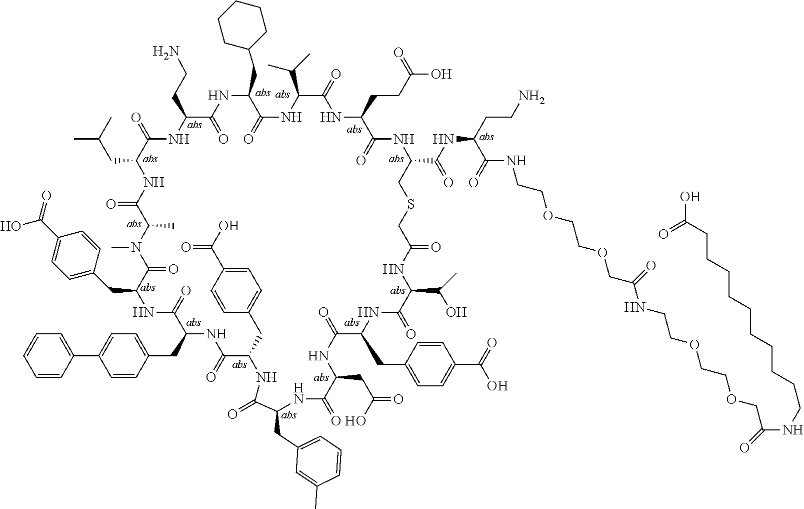 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2538 was prepared on a 50 μmol scale. The yield of the product was 16.6 mg, and its estimated purity by LCMS analysis was 100%. Analysis condition A: Retention time=1.42 min; ESI-MS(+) m/z [M+2H]²⁺: 1295.2.

### Preparation of Example 2539

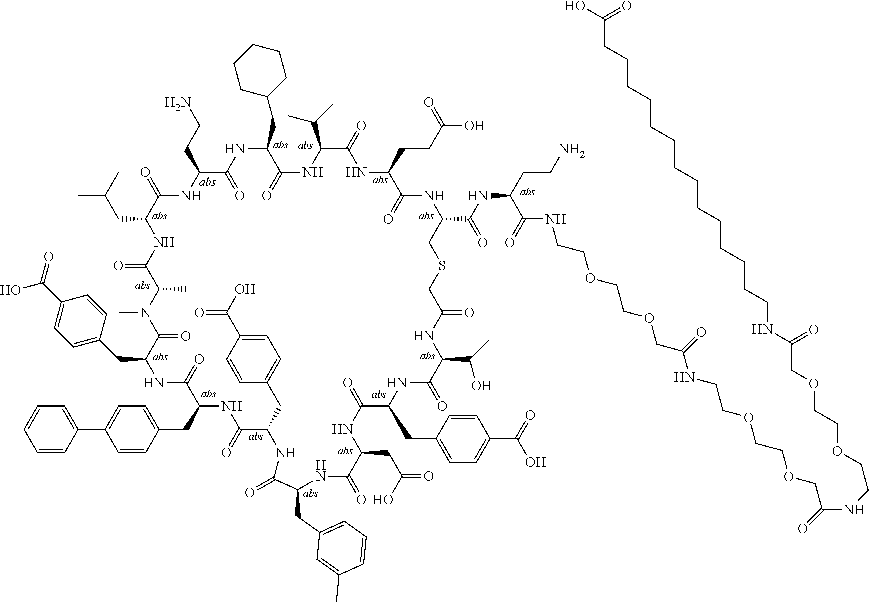 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCOCCOCC(=O)NCCCCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2539 was prepared on a 50 μmol scale. The yield of the product was 23.5 mg, and its estimated purity by LCMS analysis was 96%. Analysis condition A: Retention time=1.72 min; ESI-MS(+) m/z [M+2H]²⁺: 1396.

### Preparation of Example 2540

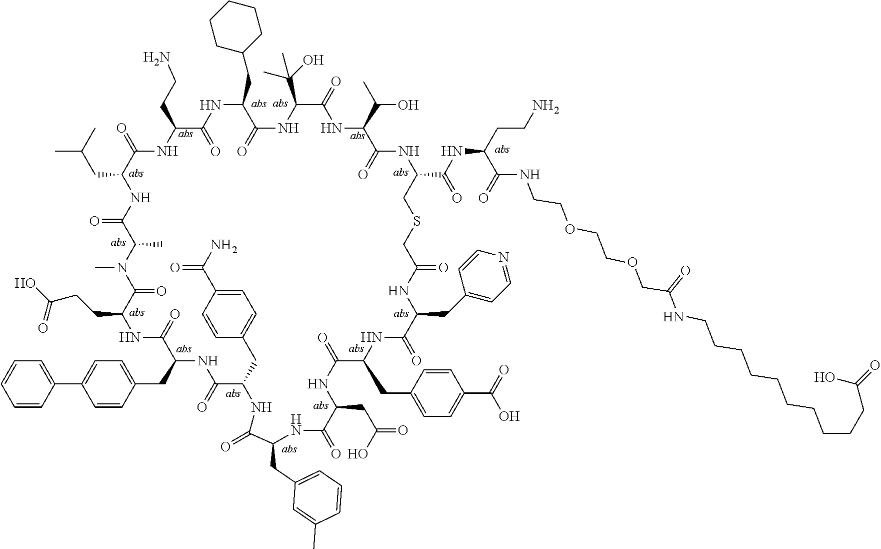 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCC(=O)O)NC(=O)[C@H](Cc3ccc(-c4ccccc4)cc3)NC(=O)[C@H](Cc3ccc(C(N)=O)cc3)NC2=O)c1]

Example 2540 was prepared on a 50 μmol scale. The yield of the product was 82.6 mg, and its estimated purity by LCMS analysis was 95.5%. Analysis condition B: Retention time=1.72 min; ESI-MS(+) m/z [M+3H]³⁺: 806.2.

### Preparation of Example 2541

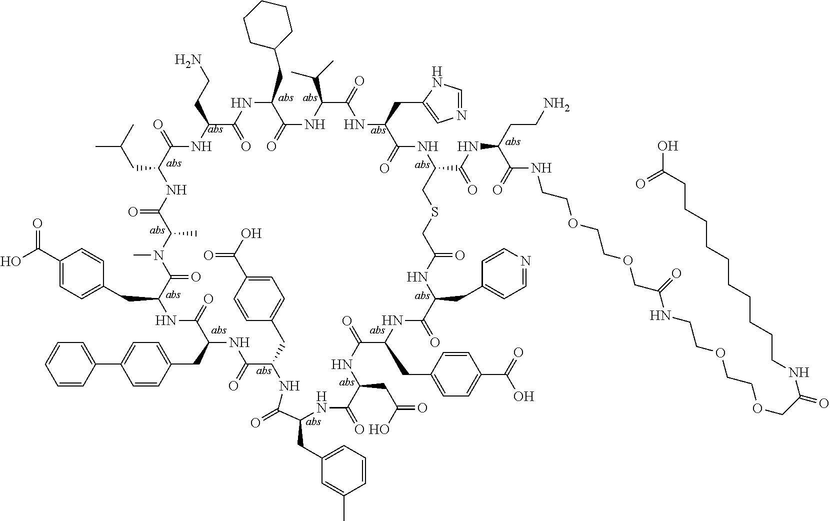 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2541 was prepared on a 50 μmol scale. The yield of the product was 16.1 mg, and its estimated purity by LCMS analysis was 96.6%. Analysis condition B: Retention time=1.78 min; ESI-MS(+) m/z [M+3H]³⁺: 882.3.

### Preparation of Example 2542

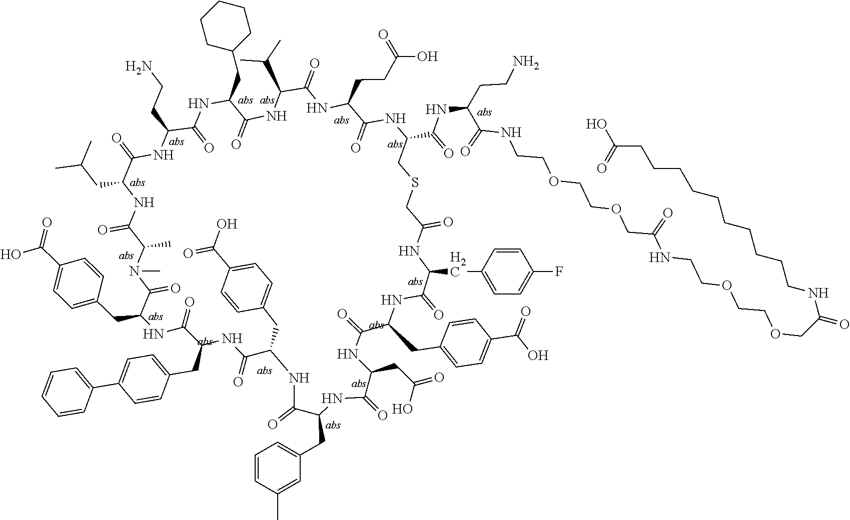 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(F)cc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2542 was prepared on a 50 μmol scale. The yield of the product was 69.2 mg, and its estimated purity by LCMS analysis was 96%. Analysis condition B: Retention time=1.86 min; ESI-MS(+) m/z [M+2H]²⁺: 1327.1.

### Preparation of Example 2543

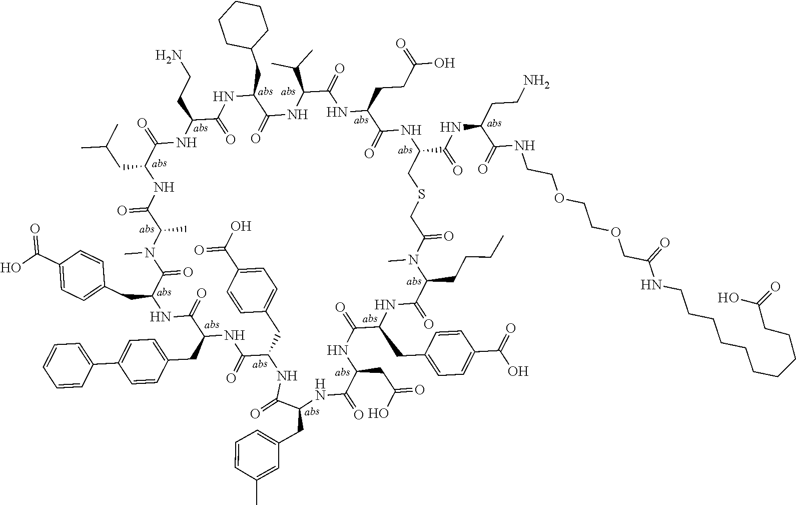 [SMILES: CCCC[C@H]1C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N[C@H](C(=O)N[C@@H](CCN)C(=O)NCCOCCOCC(=O)NCCCCCCCCCCC(=O)O)CSCC(=O)N1C]

Example 2543 was prepared on a 50 μmol scale. The yield of the product was 17.1 mg, and its estimated purity by LCMS analysis was 98.7%. Analysis condition B: Retention time=2.01 min; ESI-MS(+) m/z [M+2H]²⁺: 1235.8.

### Preparation of Example 2544

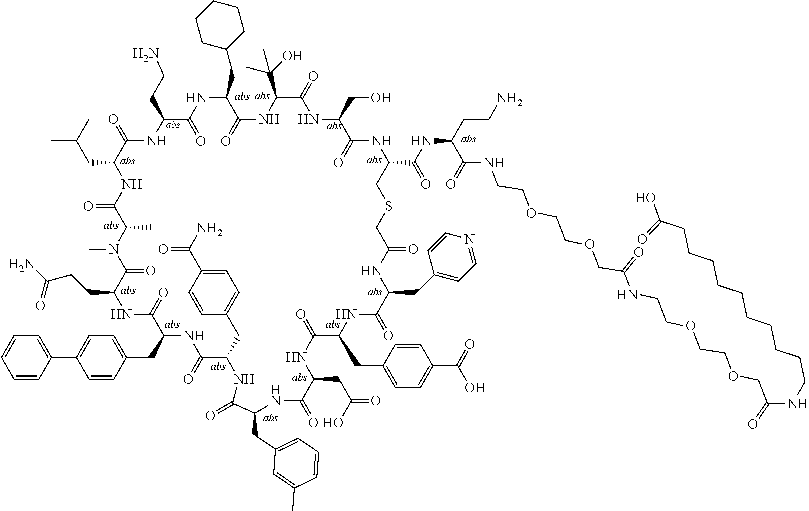 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCC(N)=O)NC(=O)[C@H](Cc3ccc(-c4ccccc4)cc3)NC(=O)[C@H](Cc3ccc(C(N)=O)cc3)NC2=O)c1]

Example 2544 was prepared on a 50 μmol scale. The yield of the product was 13.1 mg, and its estimated purity by LCMS analysis was 95.3%. Analysis condition A: Retention time=1.7 min; ESI-MS(+) m/z [M+2H]²⁺: 1274.

### Preparation of Example 2545

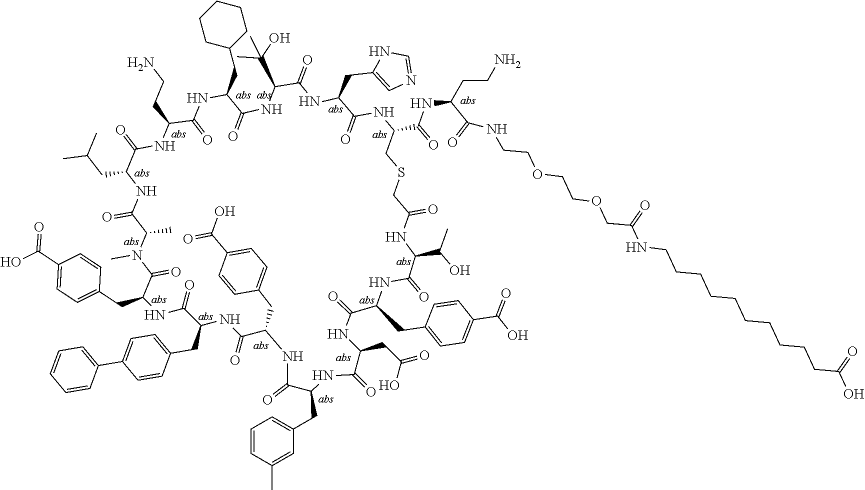 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2545 was prepared on a 50 μmol scale. The yield of the product was 18.8 mg, and its estimated purity by LCMS analysis was 95.1%. Analysis condition B: Retention time=1.8 min; ESI-MS(+) m/z [M+2H]²⁺: 1234.2.

### Preparation of Example 2546

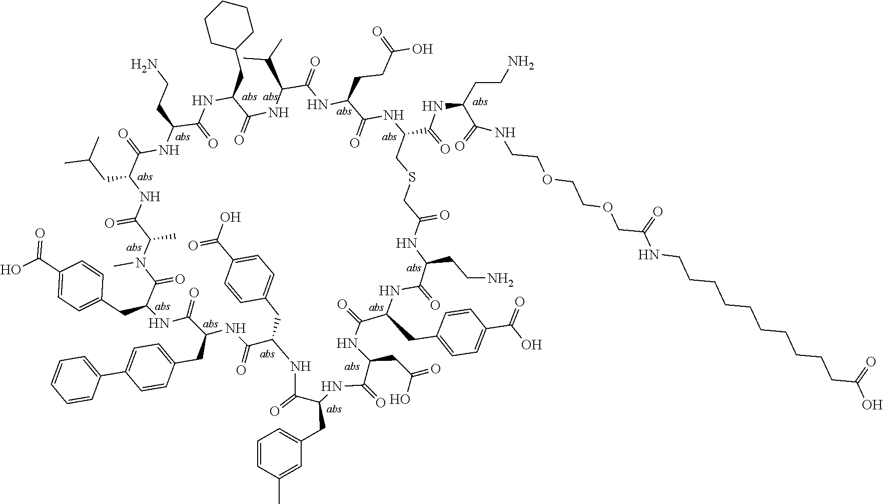 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2546 was prepared on a 50 μmol scale. The yield of the product was 24 mg, and its estimated purity by LCMS analysis was 100%. Analysis condition B: Retention time=1.79 min; ESI-MS(+) m/z [M+3H]³⁺: 815.1.

### Preparation of Example 2547

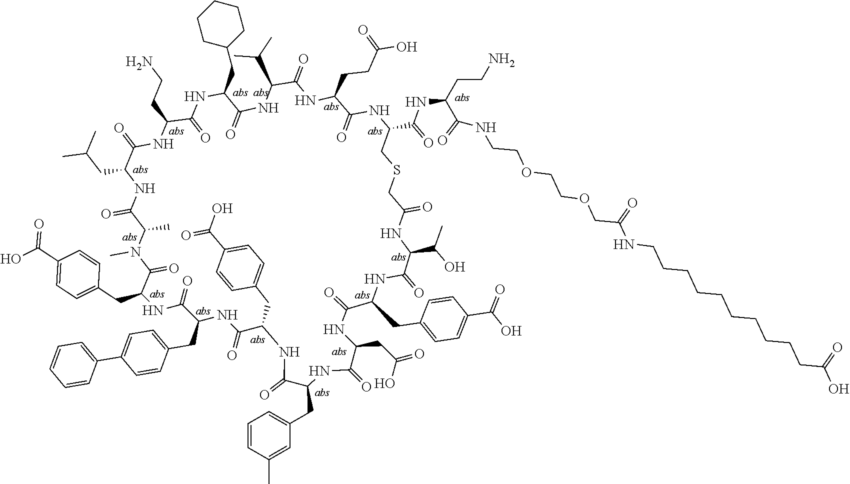 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2547 was prepared on a 50 μmol scale. The yield of the product was 14 mg, and its estimated purity by LCMS analysis was 96%. Analysis condition A: Retention time=1.53 min; ESI-MS(+) m/z [M+2H]²⁺: 1222.2.

### Preparation of Example 2548

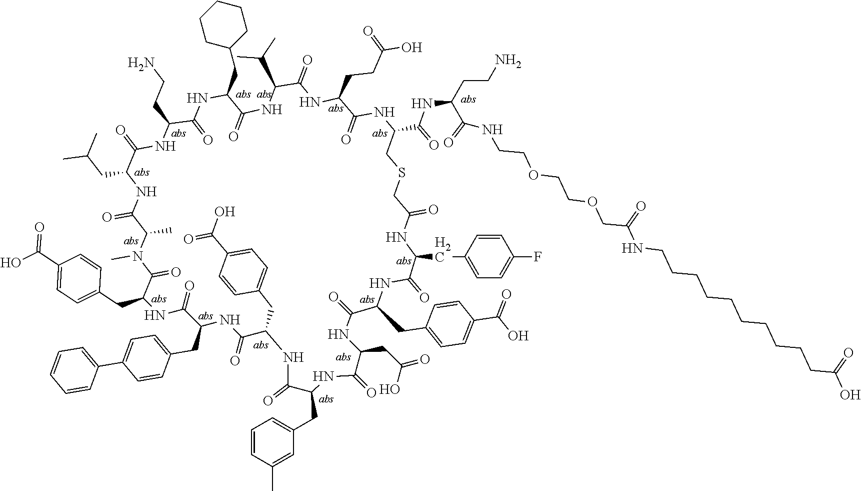 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(F)cc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2548 was prepared on a 50 μmol scale. The yield of the product was 16.5 mg, and its estimated purity by LCMS analysis was 95.8%. Analysis condition A: Retention time=1.67 min; ESI-MS(+) m/z [M+2H]²⁺: 1254.

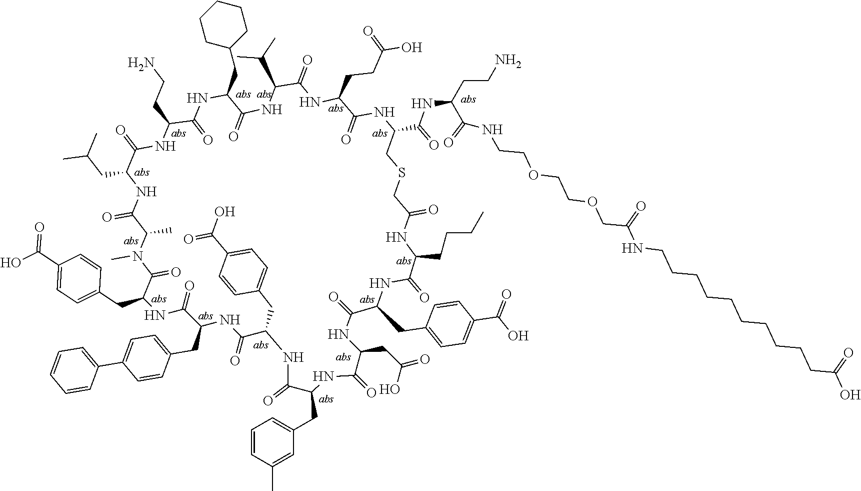 [SMILES: CCCC[C@@H]1NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Example 2549 was prepared on a 50 μmol scale. The yield of the product was 18.3 mg, and its estimated purity by LCMS analysis was 99%. Analysis condition B: Retention time=1.95 min; ESI-MS(+) m/z [M+2H]²⁺: 1228.3.

### Preparation of Example 2550

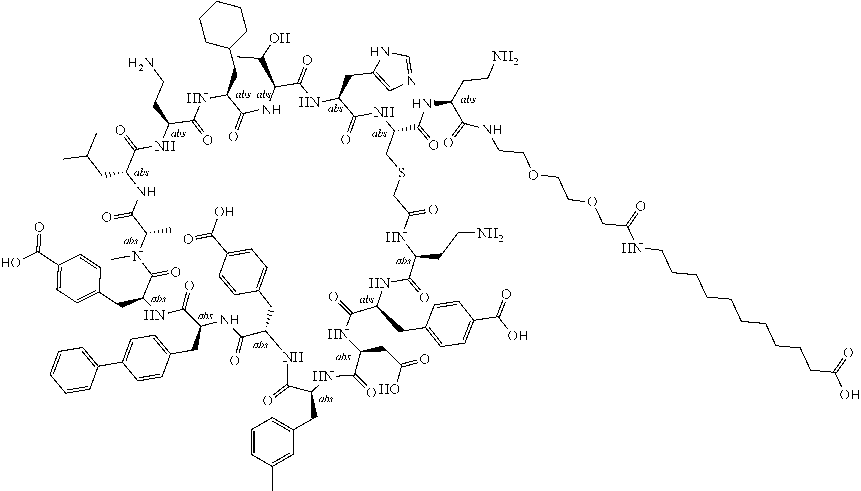 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2550 was prepared on a 50 μmol scale. The yield of the product was 55.1 mg, and its estimated purity by LCMS analysis was 94.1%. Analysis condition A: Retention time=1.61 min; ESI-MS(+) m/z [M+2H]²⁺: 1226.9.

### Preparation of Example 2551

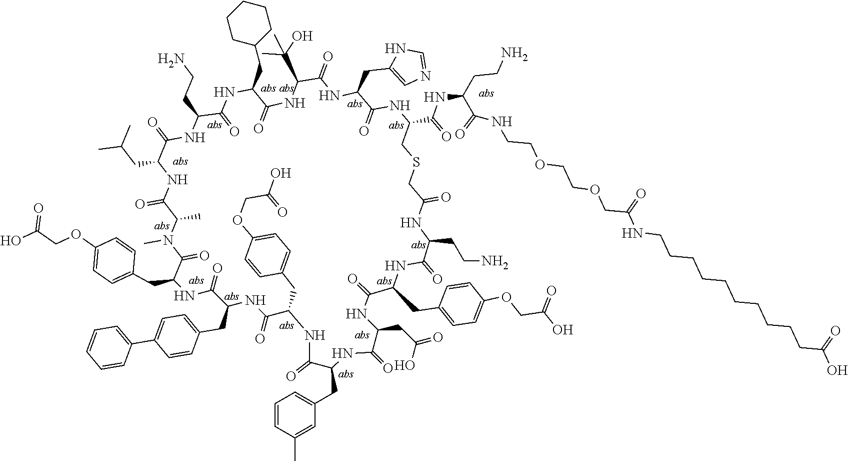 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2551 was prepared on a 50 μmol scale. The yield of the product was 3.5 mg, and its estimated purity by LCMS analysis was 100%. Analysis condition A: Retention time=1.62 min; ESI-MS(+) m/z [M+2H]²⁺: 1279.1.

### Preparation of Example 2552

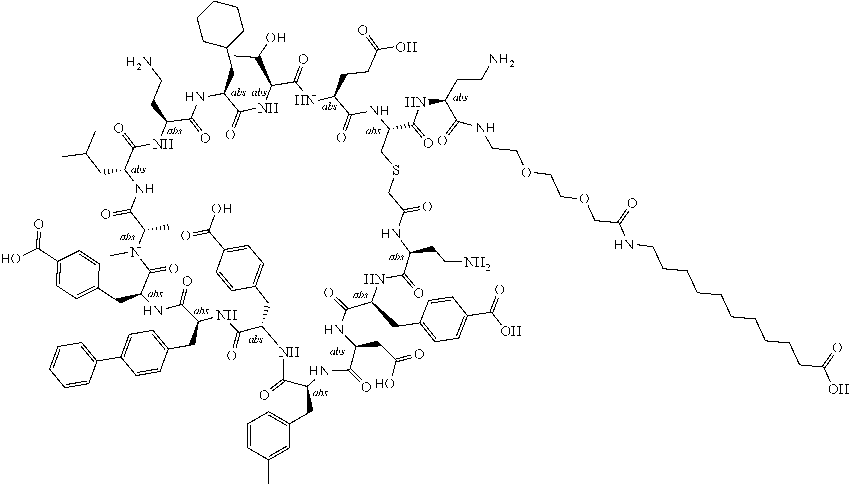 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2552 was prepared on a 50 μmol scale. The yield of the product was 14.6 mg, and its estimated purity by LCMS analysis was 100%. Analysis condition B: Retention time=1.76 min; ESI-MS(+) m/z [M+2H]²⁺: 1223.2.

### Preparation of Example 2553

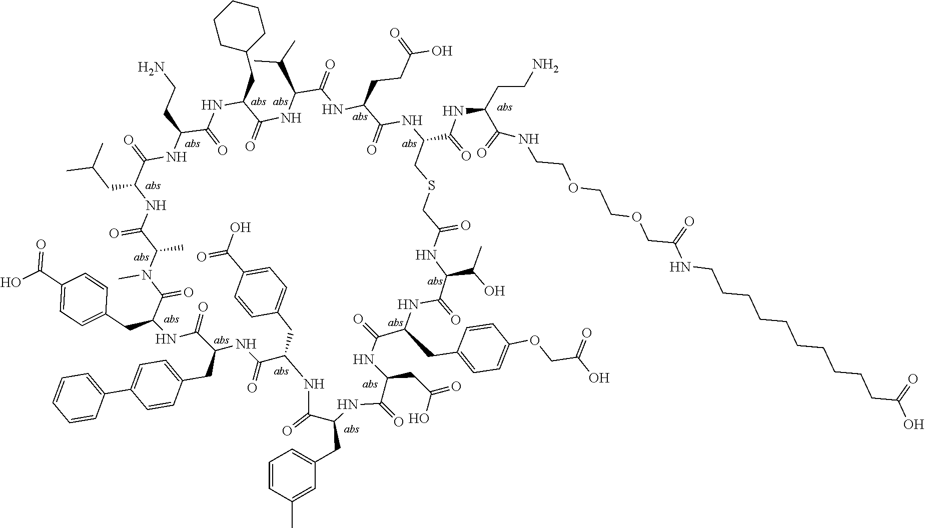 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2553 was prepared on a 50 μmol scale. The yield of the product was 24.6 mg, and its estimated purity by LCMS analysis was 95.3%. Analysis condition A: Retention time=1.51 min; ESI-MS(+) m/z [M+2H]²⁺: 1237.2.

### Preparation of Example 2554

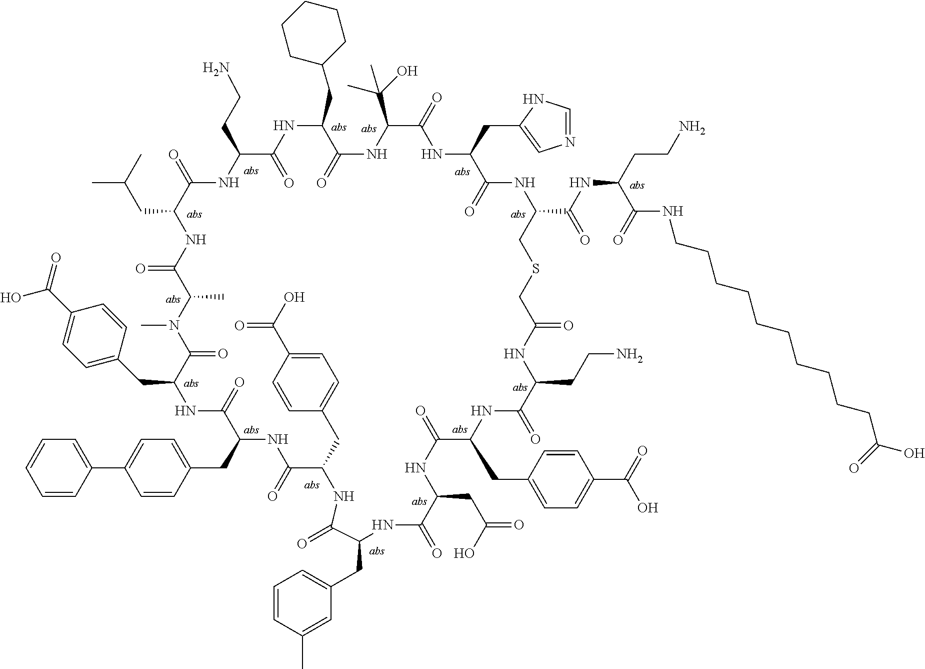 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2554 was prepared on a 50 μmol scale. The yield of the product was 51 mg, and its estimated purity by LCMS analysis was 97.3%. Analysis condition B: Retention time=1.68 min; ESI-MS(+) m/z [M+2H]²⁺: 1161.1.

### Preparation of Example 2555

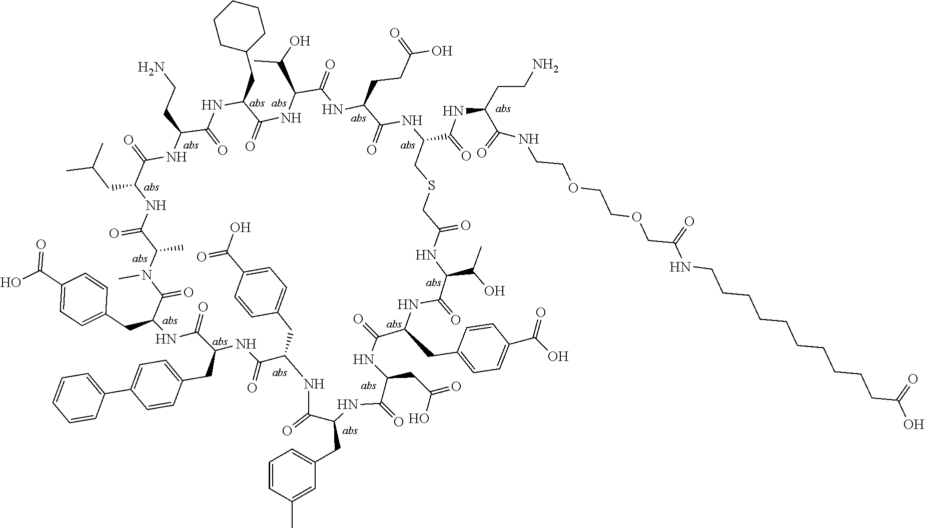 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2555 was prepared on a 50 μmol scale. The yield of the product was 8.9 mg, and its estimated purity by LCMS analysis was 100%. Analysis condition A: Retention time=1.4 min; ESI-MS(+) m/z [M+2H]²⁺: 1223.3.

### Preparation of Example 2556

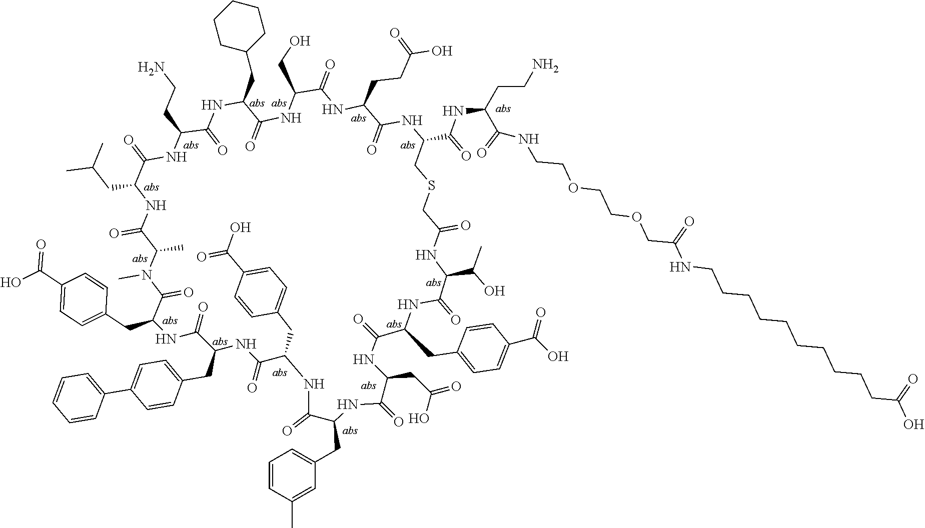 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](CO)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2556 was prepared on a 50 μmol scale. The yield of the product was 46.9 mg, and its estimated purity by LCMS analysis was 95.7%. Analysis condition A: Retention time=1.46 min; ESI-MS(+) m/z [M+2H]²⁺: 1216.3.

### Preparation of Example 2557

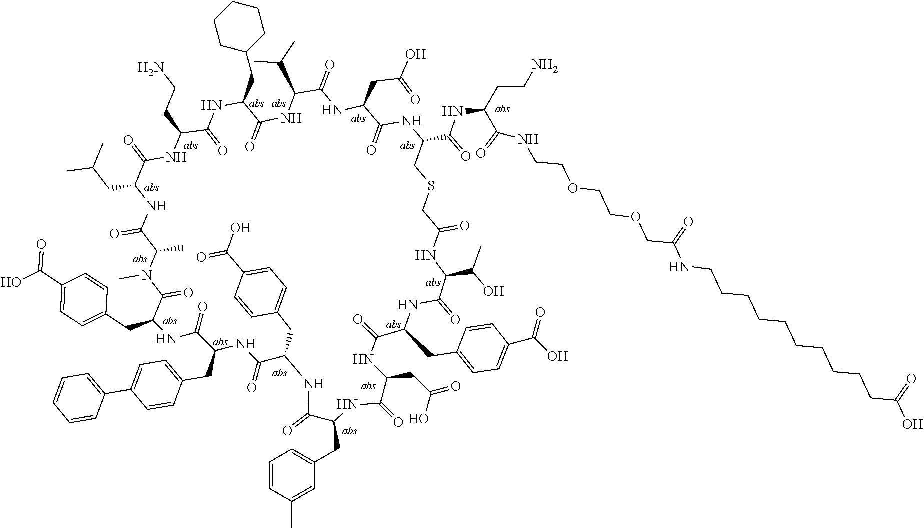 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2557 was prepared on a 50 μmol scale. The yield of the product was 16 mg, and its estimated purity by LCMS analysis was 95%. Analysis condition B: Retention time=1.87 min; ESI-MS(+) m/z [M+2H]²⁺: 1216.

### Preparation of Example 2558

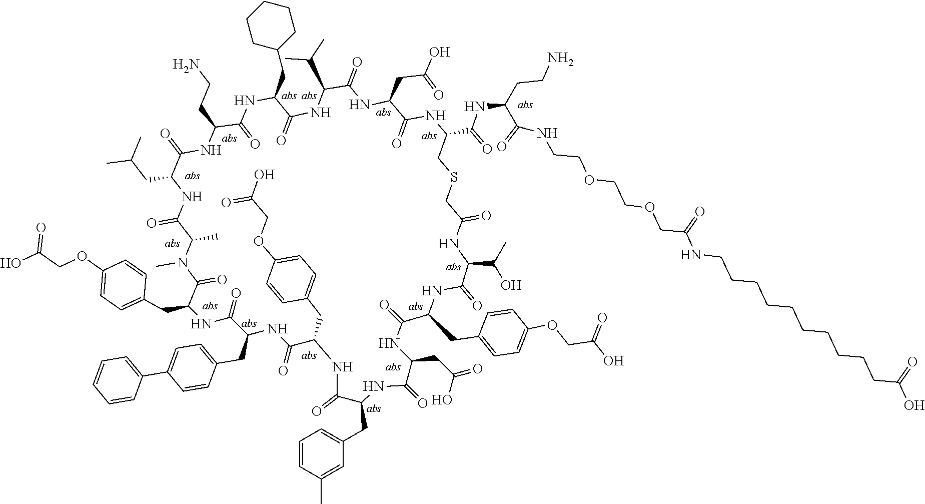 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2558 was prepared on a 50 μmol scale. The yield of the product was 58.8 mg, and its estimated purity by LCMS analysis was 100%. Analysis condition B: Retention time=1.85 min; ESI-MS(+) m/z [M+2H]²⁺: 1260.9.

### Preparation of Example 2559

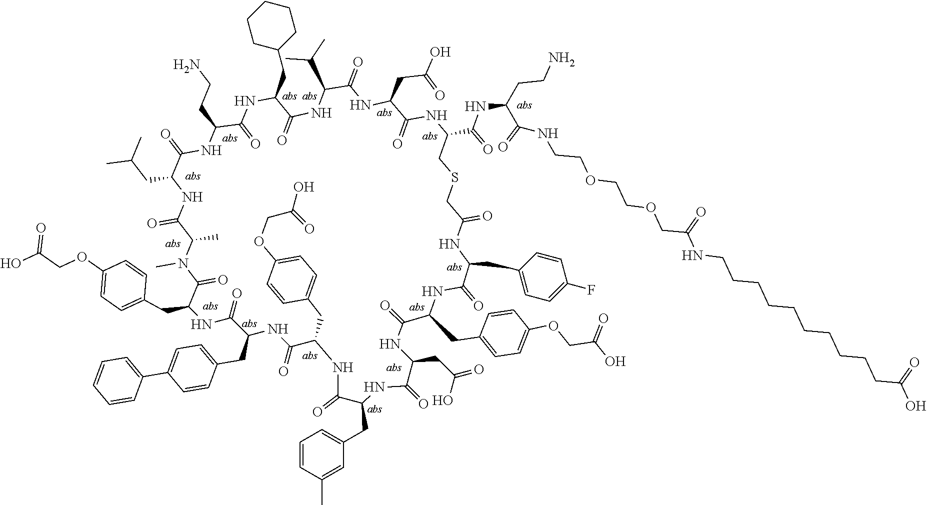 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2559 was prepared on a 50 μmol scale. The yield of the product was 40.2 mg, and its estimated purity by LCMS analysis was 96.6%. Analysis condition A: Retention time=1.57 min; ESI-MS(+) m/z [M+2H]²⁺: 1292.9.

### Preparation of Example 2560

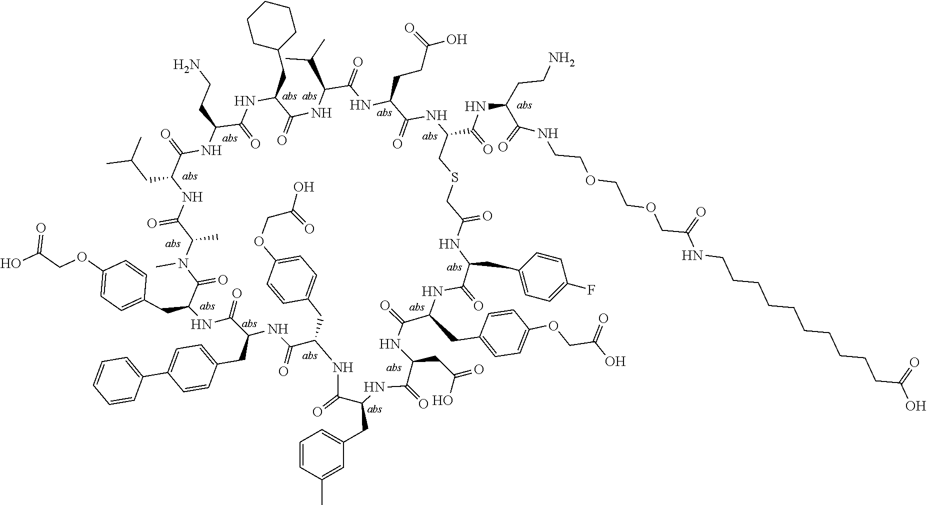 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2560 was prepared on a 50 μmol scale. The yield of the product was 14.5 mg, and its estimated purity by LCMS analysis was 95.1%. Analysis condition A: Retention time=1.6 min; ESI-MS(+) m/z [M+2H]²⁺: 1300.1.

### Preparation of Example 2561

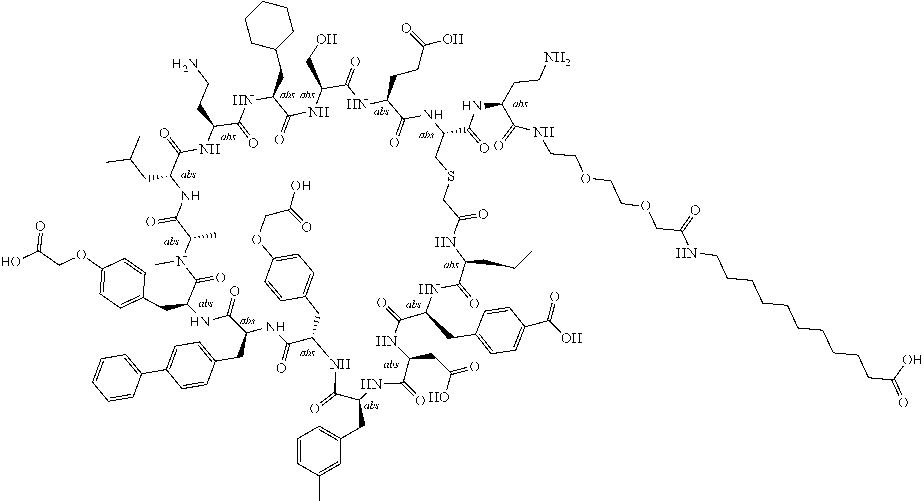 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](CO)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Example 2561 was prepared on a 50 μmol scale. The yield of the product was 29.6 mg, and its estimated purity by LCMS analysis was 97.9%. Analysis condition B: Retention time=1.98 min; ESI-MS(+) m/z [M+2H]²⁺: 1245.3.

### Preparation of Example 2562

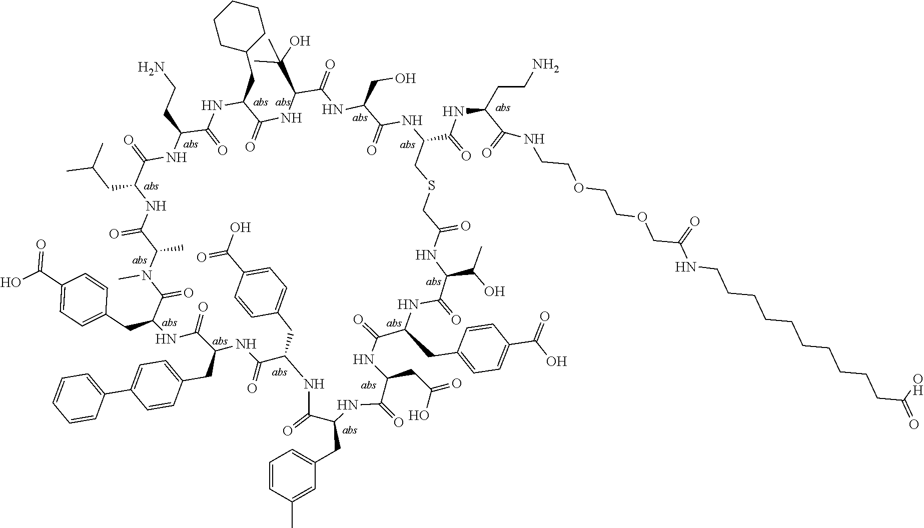 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2562 was prepared on a 50 μmol scale. The yield of the product was 17.3 mg, and its estimated purity by LCMS analysis was 98.9%. Analysis condition B: Retention time=1.92 min; ESI-MS(+) m/z [M+2H]²⁺: 1209.3.

### Preparation of Example 2563

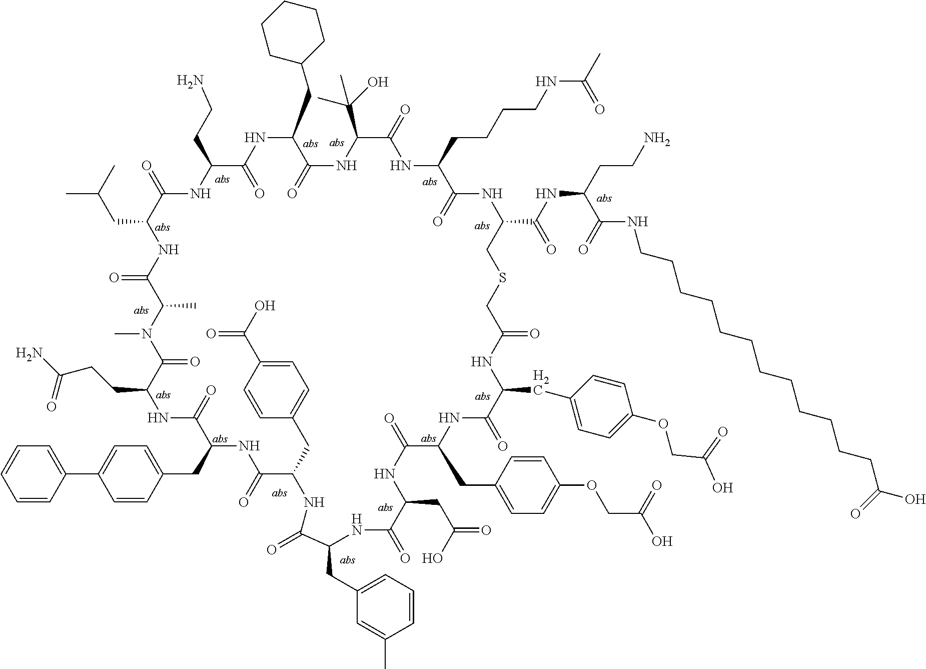 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2563 was prepared on a 50 μmol scale. The yield of the product was 40.4 mg, and its estimated purity by LCMS analysis was 94.5%. Analysis condition B: Retention time=1.73 min; ESI-MS(+) m/z [M+2H]²⁺: 1236.2.

### Preparation of Example 2564

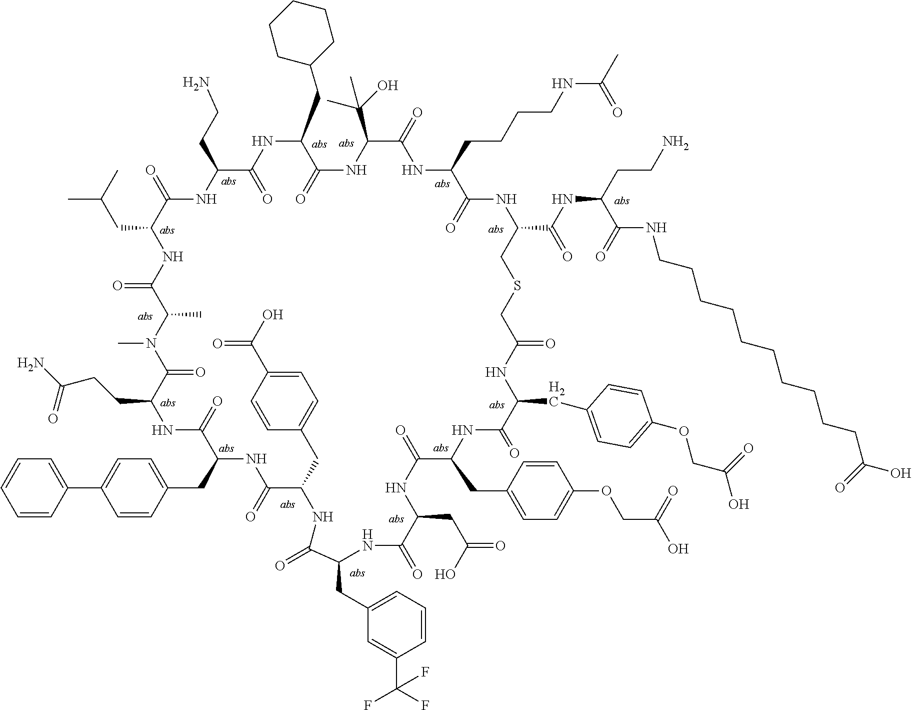 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC1=O]

Example 2564 was prepared on a 50 μmol scale. The yield of the product was 51.9 mg, and its estimated purity by LCMS analysis was 98.9%. Analysis condition B: Retention time=1.76 min; ESI-MS(+) m/z [M+2H]²⁺: 1249.

### Preparation of Example 2565

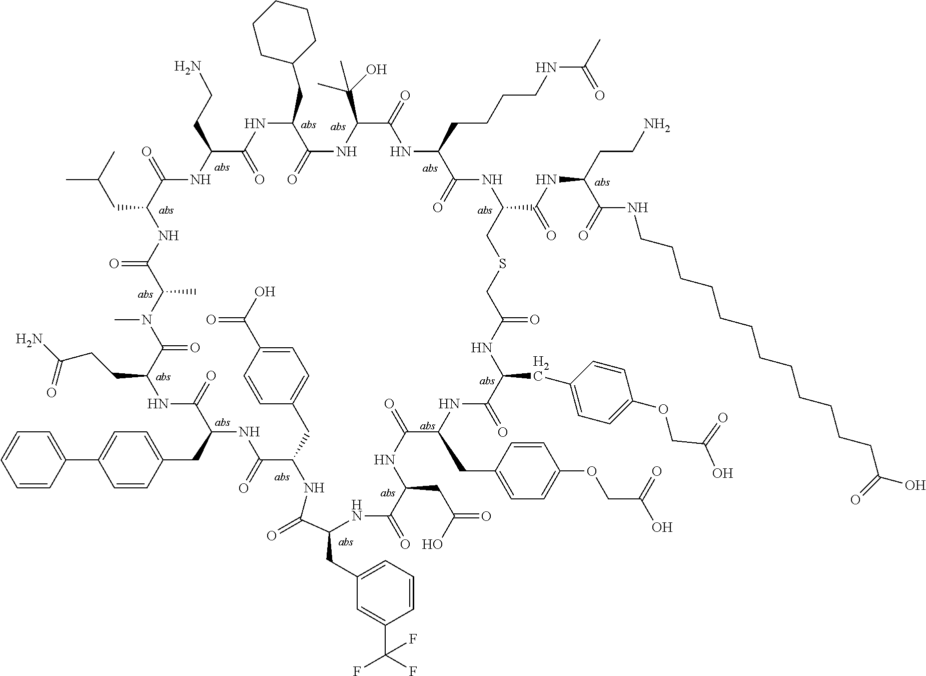 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2565 was prepared on a 50 μmol scale. The yield of the product was 48.2 mg, and its estimated purity by LCMS analysis was 97.8%. Analysis condition A: Retention time=1.62 min; ESI-MS(+) m/z [M+2H]²⁺: 1262.9.

### Preparation of Example 2566

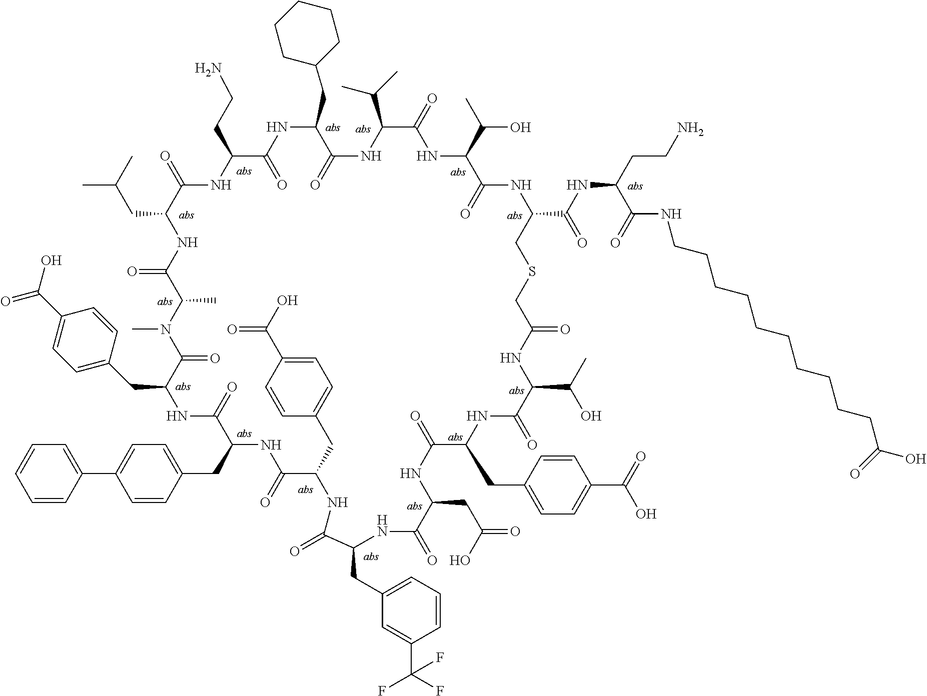 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2566 was prepared on a 50 μmol scale. The yield of the product was 23.4 mg, and its estimated purity by LCMS analysis was 95.6%. Analysis condition A: Retention time=1.52 min; ESI-MS(+) m/z [M+2H]²⁺: 1163.1.

### Preparation of Example 2567

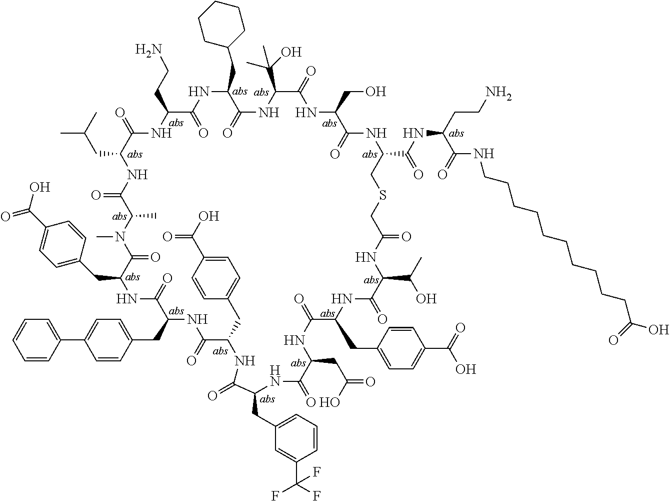 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2567 was prepared on a 50 μmol scale. The yield of the product was 19.1 mg, and its estimated purity by LCMS analysis was 95.4%. Analysis condition B: Retention time=1.79 min; ESI-MS(+) m/z [M+2H]²⁺: 1164.

### Preparation of Example 2568

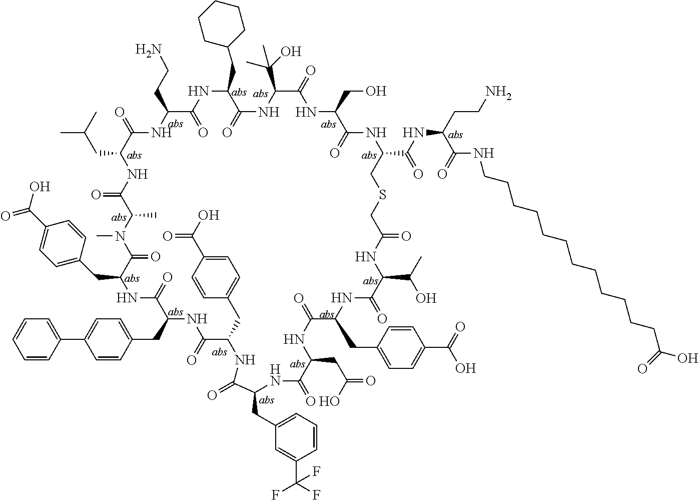 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2568 was prepared on a 50 μmol scale. The yield of the product was 20.2 mg, and its estimated purity by LCMS analysis was 95.9%. Analysis condition A: Retention time=1.68, 1.73 min; ESI-MS(+) m/z [M+2H]²⁺: 1177.82, 1177.88.

### Preparation of Example 2569

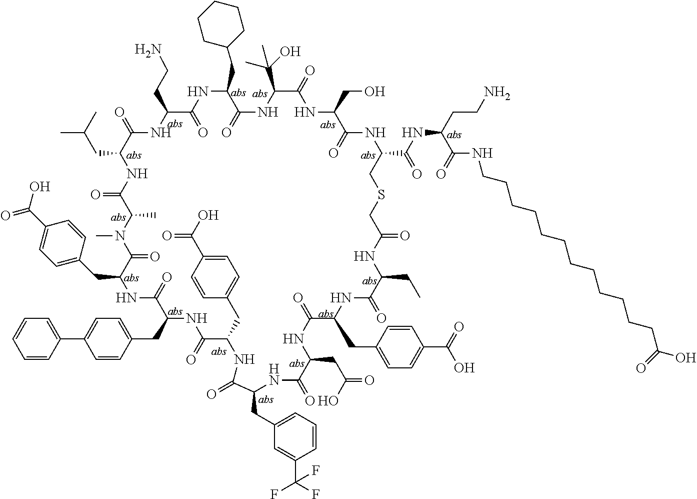 [SMILES: CC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Example 2569 was prepared on a 50 μmol scale. The yield of the product was 27.4 mg, and its estimated purity by LCMS analysis was 97.7%. Analysis condition A: Retention time=1.55 min; ESI-MS(+) m/z [M+2H]²⁺: 1170.

### Preparation of Example 2570

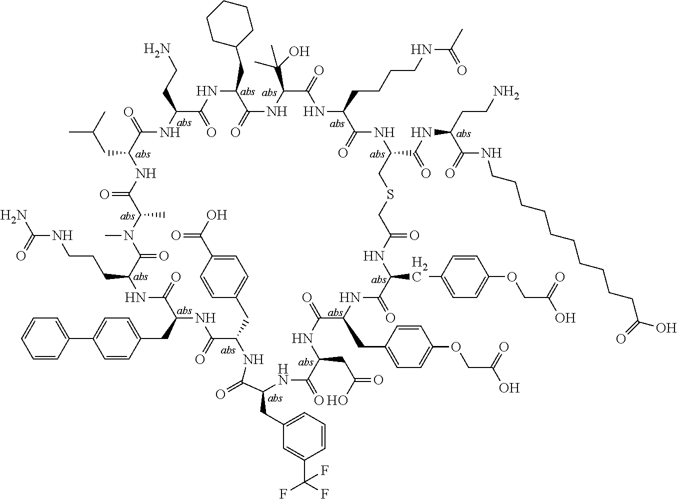 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC1=O]

Example 2570 was prepared on a 50 μmol scale. The yield of the product was 27.3 mg, and its estimated purity by LCMS analysis was 96.6%. Analysis condition A: Retention time=1.46 min; ESI-MS(+) m/z [M+2H]²⁺: 1263.4.

### Preparation of Example 2571

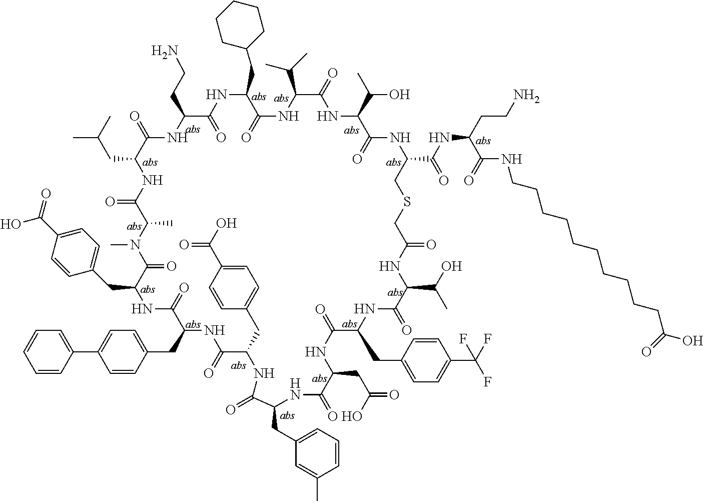 [SMILES: Cc1cccc(C[C@@H]2NC(=O)[C@H](CC(=O)O)NC(=O)[C@H](Cc3ccc(C(F)(F)F)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2571 was prepared on a 50 μmol scale. The yield of the product was 5 mg, and its estimated purity by LCMS analysis was 99.2%. Analysis condition B: Retention time=2.14 min; ESI-MS(+) m/z [M+2H]²⁺: 1148.

### Preparation of Example 2572

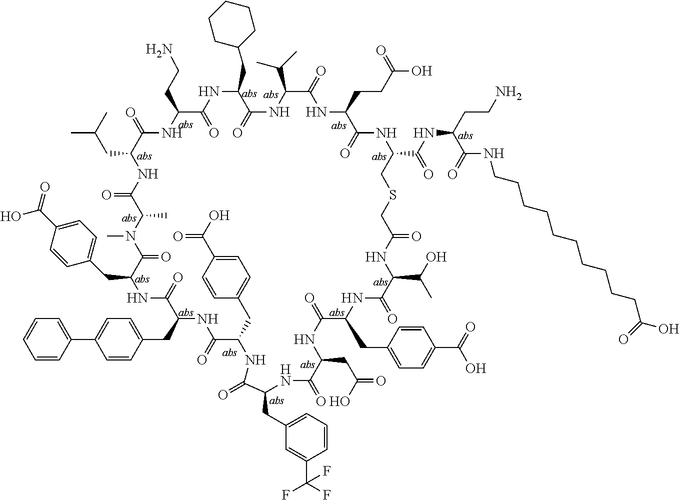 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2572 was prepared on a 50 μmol scale. The yield of the product was 21.9 mg, and its estimated purity by LCMS analysis was 95.7%. Analysis condition B: Retention time=1.88 min; ESI-MS(+) m/z [M+2H]²⁺: 1177.

### Preparation of Example 2573

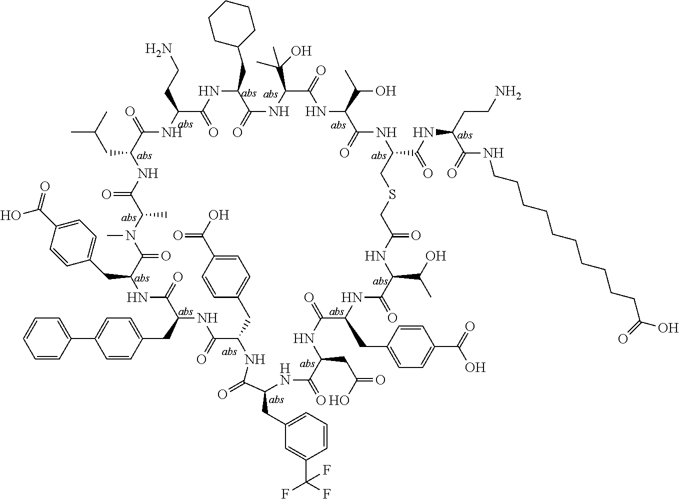 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC1=O]

Example 2573 was prepared on a 50 μmol scale. The yield of the product was 56.1 mg, and its estimated purity by LCMS analysis was 94.2%. Analysis condition A: Retention time=1.5 min; ESI-MS(+) m/z [M+2H]²⁺: 1171.1.

### Preparation of Example 2574

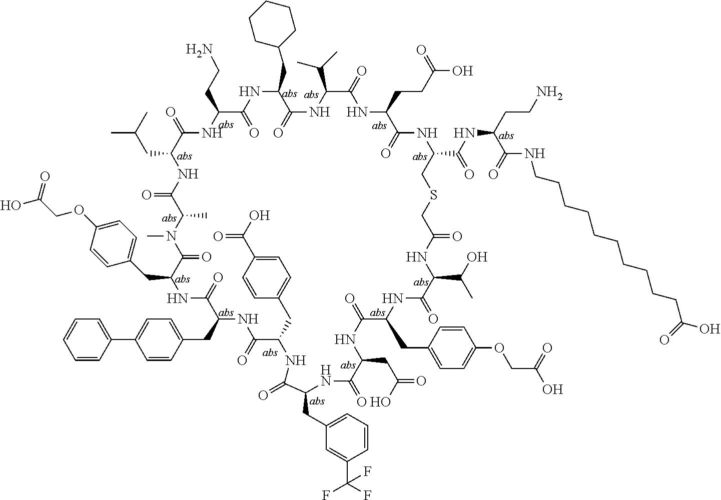 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2574 was prepared on a 50 μmol scale. The yield of the product was 19.1 mg, and its estimated purity by LCMS analysis was 88.1%. Analysis condition A: Retention time=1.42 min; ESI-MS(+) m/z [M+2H]²⁺: 1207.

### Preparation of Example 2575

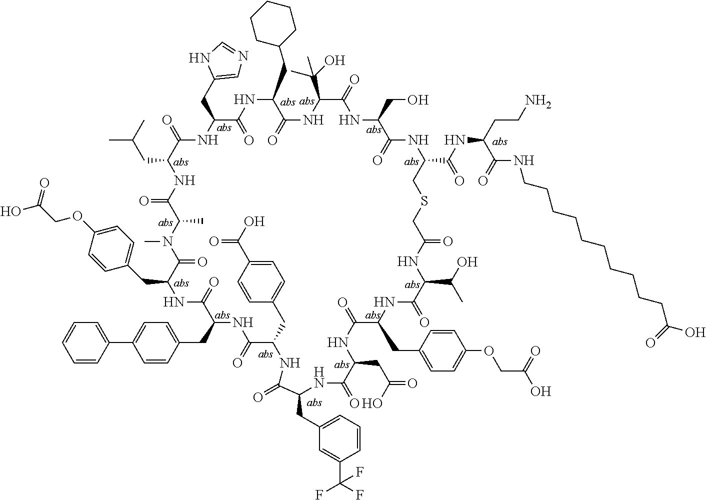 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](Cc2cnc[nH]2)NC1=O]

Example 2575 was prepared on a 50 μmol scale. The yield of the product was 24 mg, and its estimated purity by LCMS analysis was 93.9%. Analysis condition A: Retention time=1.51 min; ESI-MS(+) m/z [M+2H]²⁺: 1211.3.

### Preparation of Example 2576

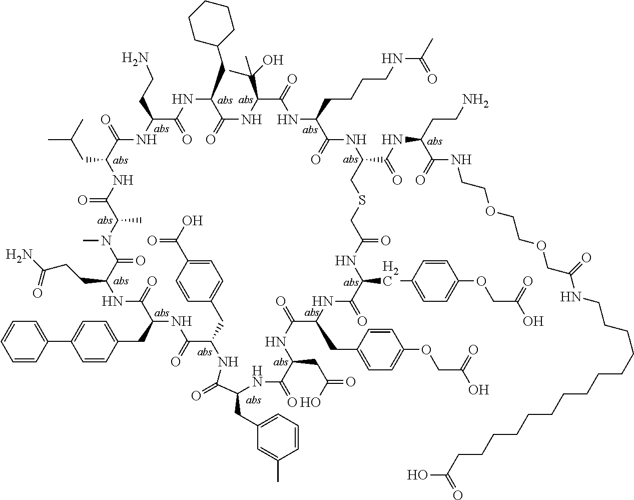 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCCCC(=O)O)NC1=O]

Example 2576 was prepared on a 50 μmol scale. The yield of the product was 27.6 mg, and its estimated purity by LCMS analysis was 93.1%. Analysis condition A: Retention time=1.57 min; ESI-MS(+) m/z [M+2H]²⁺: 1323.3.

### Preparation of Example 2577

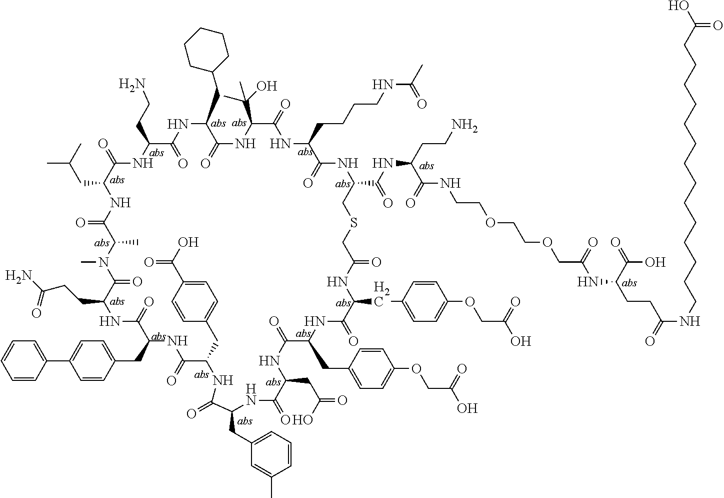 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@@H](CCC(=O)NCCCCCCCCCCCCCCC(=O)O)C(=O)O)NC1=O]

Example 2577 was prepared on a 50 μmol scale. The yield of the product was 32.3 mg, and its estimated purity by LCMS analysis was 100%. Analysis condition B: Retention time=2.02 min; ESI-MS(+) m/z [M+2H]²⁺: 1387.1.

### Preparation of Example 2578

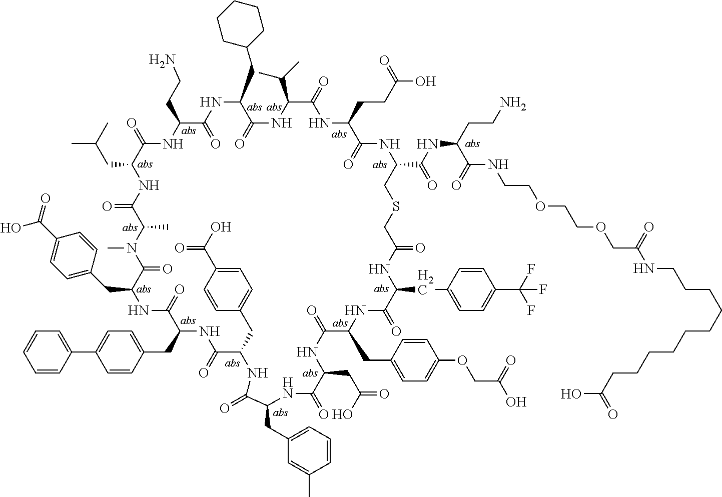

Example 2578 was prepared on a 50 μmol scale. The yield of the product was 45.6 mg, and its estimated purity by LCMS analysis was 92.5%. Analysis condition A: Retention time=1.71 min; ESI-MS(+) m/z [M+2H]²⁺: 1295.2.

### Preparation of Example 2579

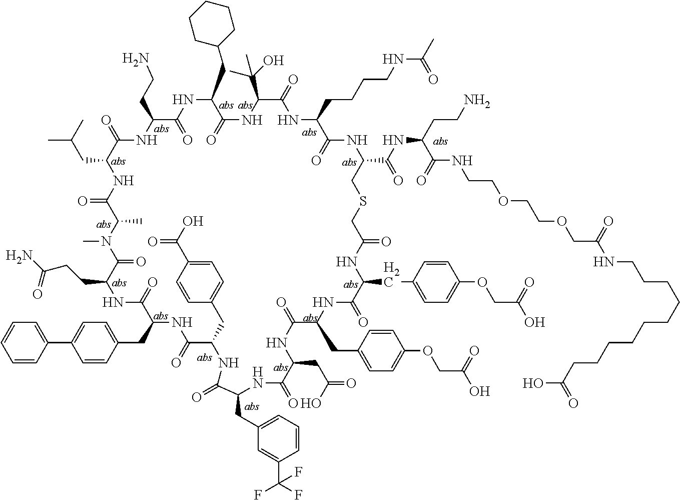

Example 2579 was prepared on a 50 μmol scale. The yield of the product was 54.5 mg, and its estimated purity by LCMS analysis was 91.2%. Analysis condition A: Retention time=1.62 min; ESI-MS(+) m/z [M+2H]²⁺: 1322.

### Preparation of Example 2580

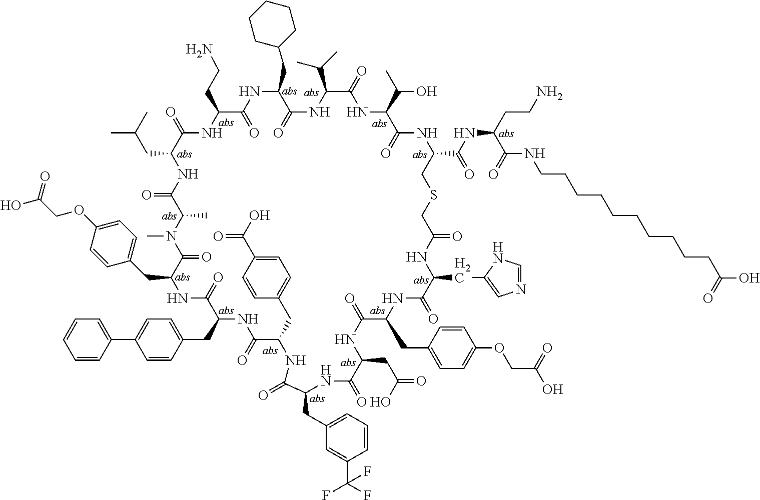 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Example 2580 was prepared on a 50 μmol scale. The yield of the product was 58.2 mg, and its estimated purity by LCMS analysis was 91.1%. Analysis condition A: Retention time=1.56 min; ESI-MS(+) m/z [M+2H]²⁺: 1211.1.

### Preparation of Example 2581

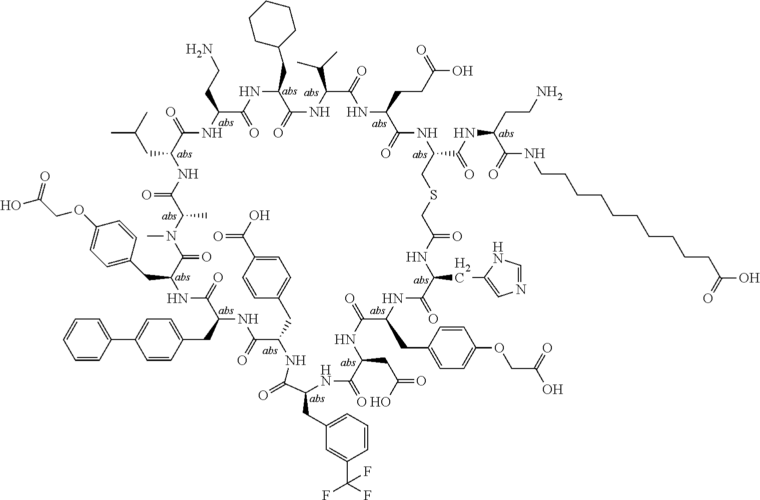 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2581 was prepared on a 50 μmol scale. The yield of the product was 6.8 mg, and its estimated purity by LCMS analysis was 100%. Analysis condition B: Retention time=1.7 min; ESI-MS(+) m/z [M+2H]²⁺: 1225.

### Preparation of Example 2582

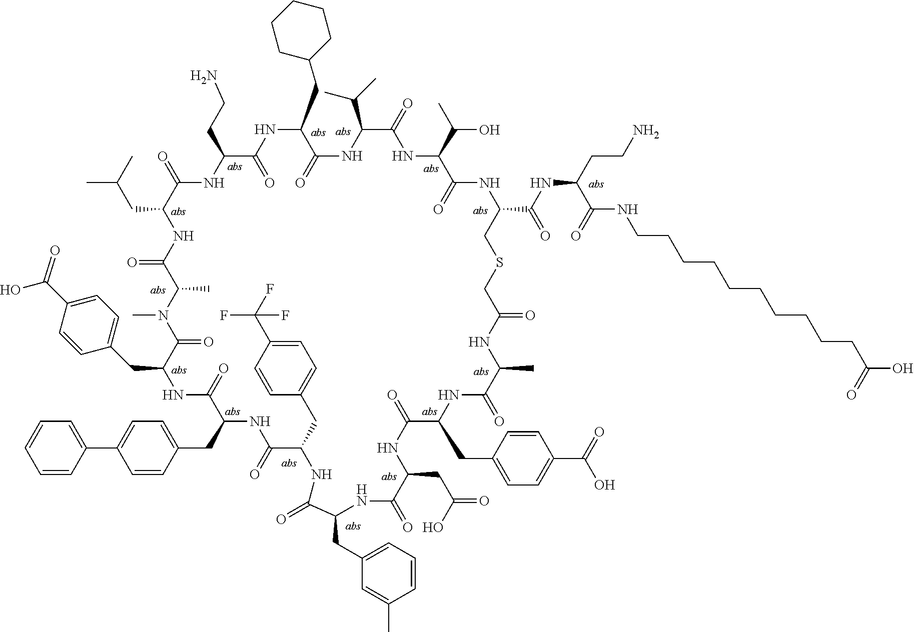 [SMILES: Cc1cccc(C[C@@H]2NC(=O)[C@H](CC(=O)O)NC(=O)[C@H](Cc3ccc(C(=O)O)cc3)NC(=O)[C@H](C)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(C(=O)O)cc3)NC(=O)[C@H](Cc3ccc(-c4ccccc4)cc3)NC(=O)[C@H](Cc3ccc(C(F)(F)F)cc3)NC2=O)c1]

Example 2582 was prepared on a 50 μmol scale. The yield of the product was 34.6 mg, and its estimated purity by LCMS analysis was 94.5%. Analysis condition A: Retention time=1.78 min; ESI-MS(+) m/z [M+2H]²⁺: 1133.1.

### Preparation of Example 2583

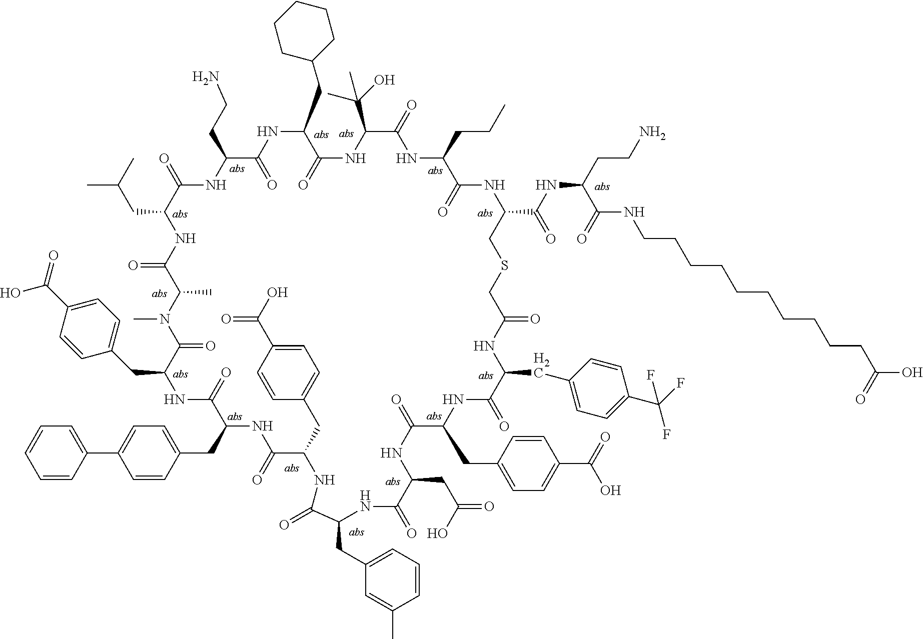 [SMILES: CCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(C(F)(F)F)cc2)NC(=O)CSC[C@@H](C(=O)N[C@@H](CCN)C(=O)NCCCCCCCCCCC(=O)O)NC1=O]

Example 2583 was prepared on a 50 μmol scale. The yield of the product was 23.5 mg, and its estimated purity by LCMS analysis was 100%. Analysis condition A: Retention time=1.82 min; ESI-MS(+) m/z [M+2H]²⁺: 1199.9.

### Preparation of Example 2584

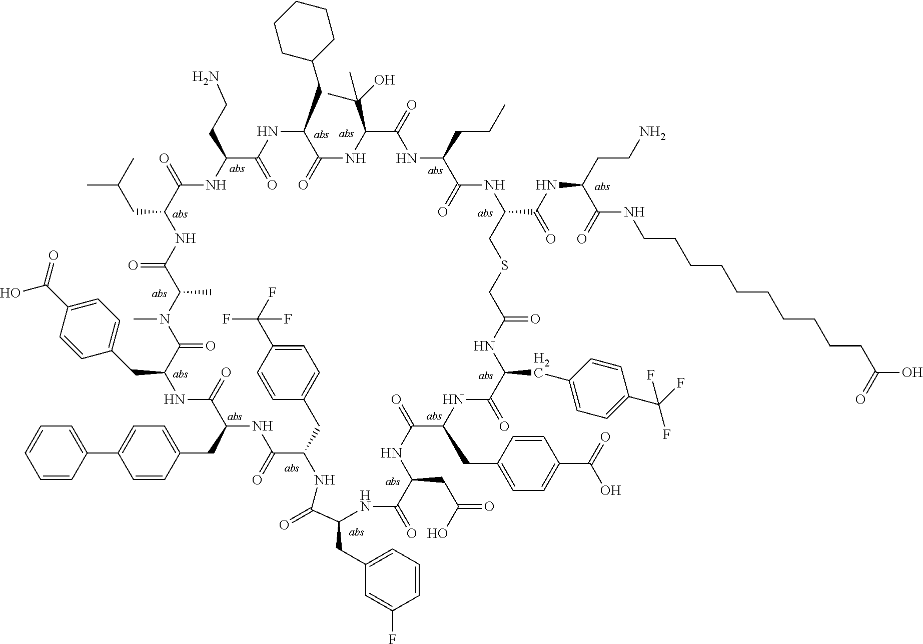 [SMILES: CCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(F)(F)F)cc2)NC(=O)[C@H](Cc2cccc(F)c2)NC(=O)[C@H](CC(=O)O)NC(=O)[C@H](Cc2ccc(C(=O)O)cc2)NC(=O)[C@H](Cc2ccc(C(F)(F)F)cc2)NC(=O)CSC[C@@H](C(=O)N[C@@H](CCN)C(=O)NCCCCCCCCCCC(=O)O)NC1=O]

Example 2584 was prepared on a 50 μmol scale. The yield of the product was 23.6 mg, and its estimated purity by LCMS analysis was 96.1%. Analysis condition A: Retention time=1.59 min; ESI-MS(+) m/z [M+2H]²⁺: 1202.

### Preparation of Example 2585

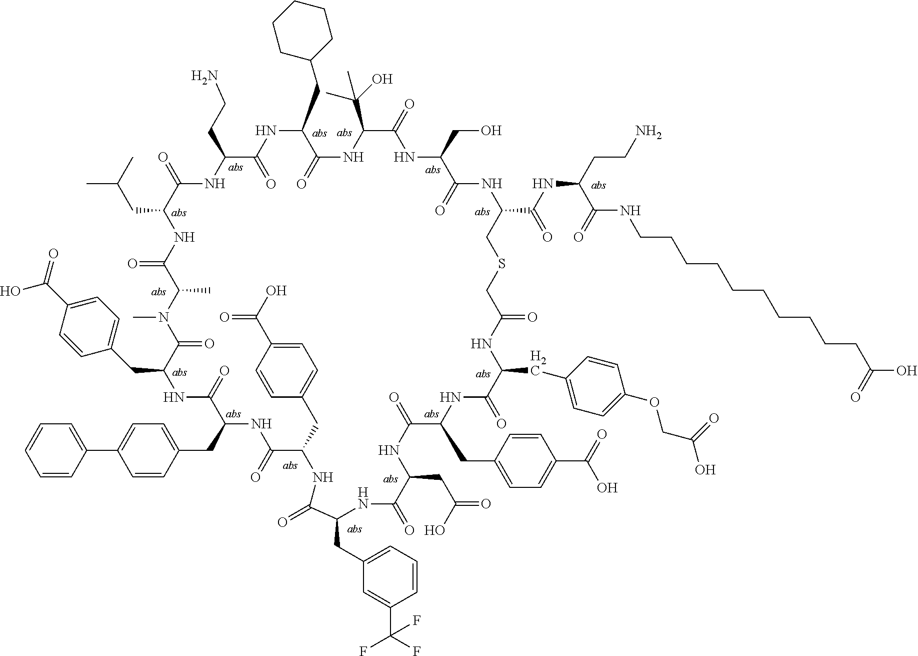 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2585 was prepared on a 50 μmol scale. The yield of the product was 33.6 mg, and its estimated purity by LCMS analysis was 90.9%. Analysis condition B: Retention time=1.58 min; ESI-MS(+) m/z [M+2H]²⁺: 1224.2.

### Preparation of Example 2586

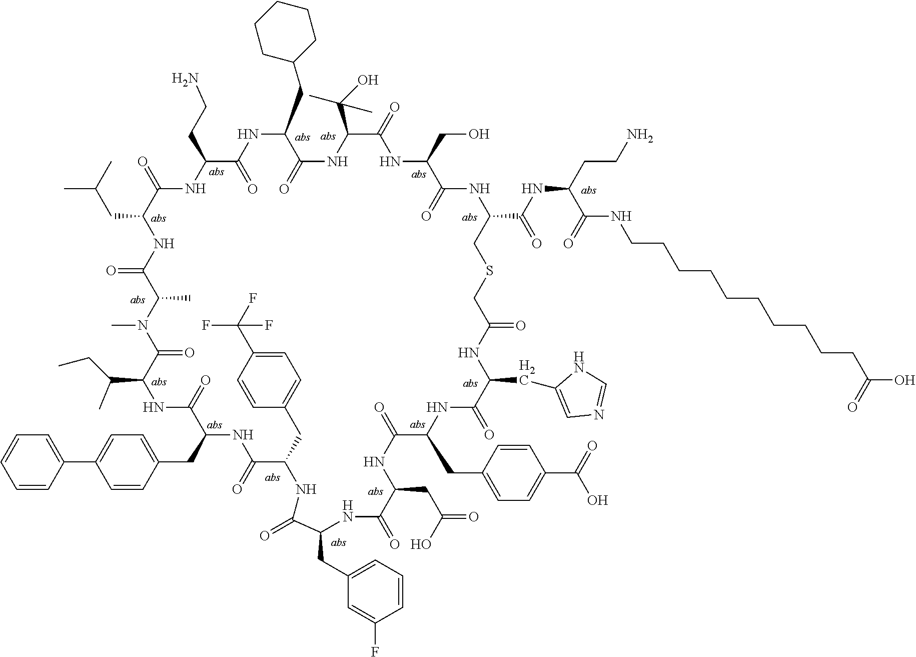 [SMILES: CCC(C)[C@@H]1NC(=O)[C@H](Cc2ccc(-c3ccccc3)cc2)NC(=O)[C@H](Cc2ccc(C(F)(F)F)cc2)NC(=O)[C@H](Cc2cccc(F)c2)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2586 was prepared on a 50 μmol scale. The yield of the product was 19.2 mg, and its estimated purity by LCMS analysis was 95.1%. Analysis condition B: Retention time=1.82 min; ESI-MS(+) m/z [M+2H]²⁺: 1130.1.

### Preparation of Example 2587

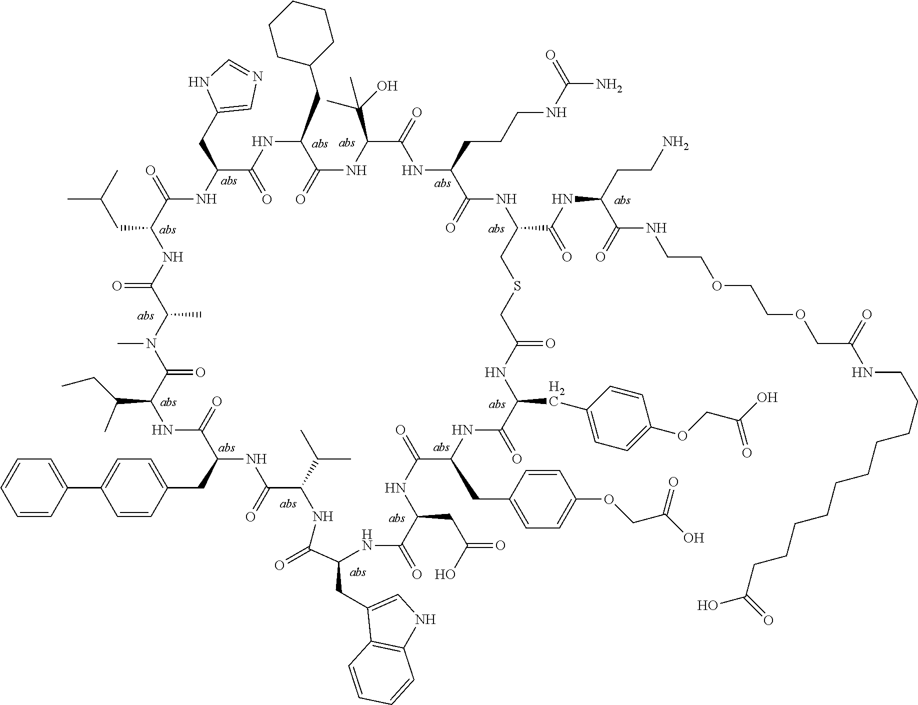 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCNC(N)=O)NC(=O)[C@H](C(C)(C)O)NC(=O)[C@H](CC2CCCCC2)NC(=O)[C@H](Cc2cnc[nH]2)NC(=O)[C@@H](CC(C)C)NC(=O)[C@H](C)N(C)C1=O]

Example 2587 was prepared on a 50 μmol scale. The yield of the product was 17.4 mg, and its estimated purity by LCMS analysis was 95.7%. Analysis condition A: Retention time=1.69 min; ESI-MS(+) m/z [M+2H]²⁺: 1265.2.

### Preparation of Example 2588

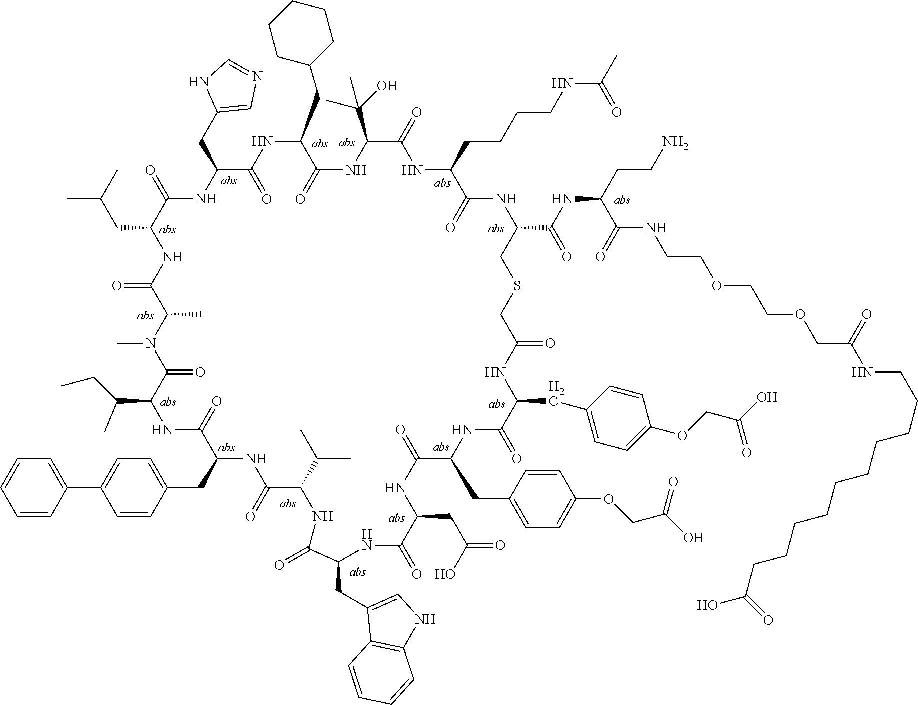 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCCNC(C)=O)NC(=O)[C@H](C(C)(C)O)NC(=O)[C@H](CC2CCCCC2)NC(=O)[C@H](Cc2cnc[nH]2)NC(=O)[C@@H](CC(C)C)NC(=O)[C@H](C)N(C)C1=O]

Example 2588 was prepared on a 50 μmol scale. The yield of the product was 27.1 mg, and its estimated purity by LCMS analysis was 96.1%. Analysis condition A: Retention time=1.55 min; ESI-MS(+) m/z [M+2H]²⁺: 1272.1.

### Preparation of Example 2589

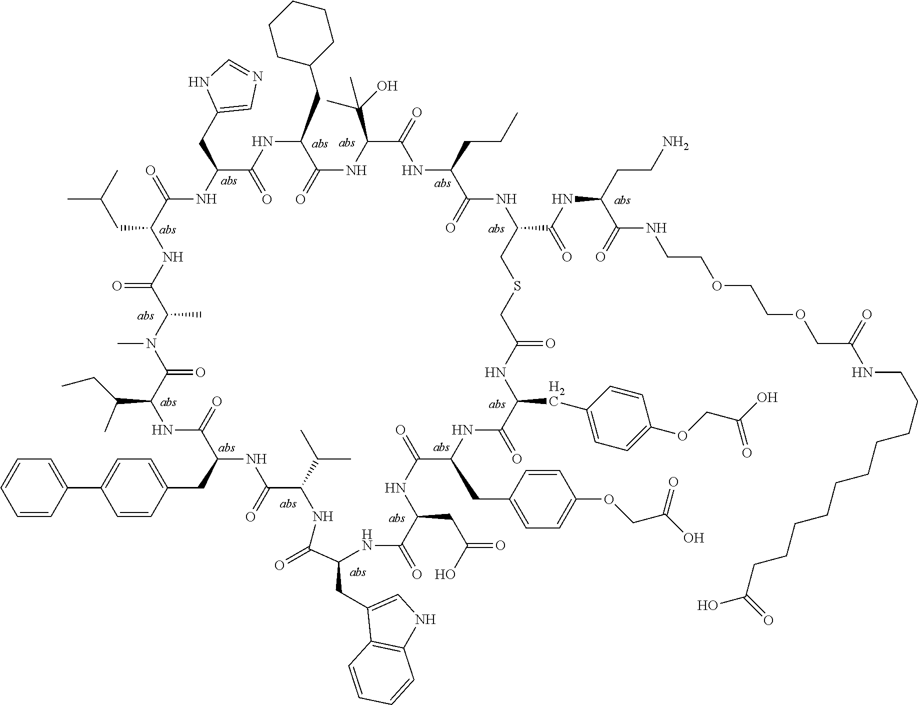 [SMILES: CCC[C@@H]1NC(=O)[C@H](C(C)(C)O)NC(=O)[C@H](CC2CCCCC2)NC(=O)[C@H](Cc2cnc[nH]2)NC(=O)[C@@H](CC(C)C)NC(=O)[C@H](C)N(C)C(=O)[C@H](C(C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2589 was prepared on a 50 μmol scale. The yield of the product was 14.1 mg, and its estimated purity by LCMS analysis was 100%. Analysis condition A: Retention time=1.6 min; ESI-MS(+) m/z [M+2H]²⁺: 1236.

### Preparation of Example 2590

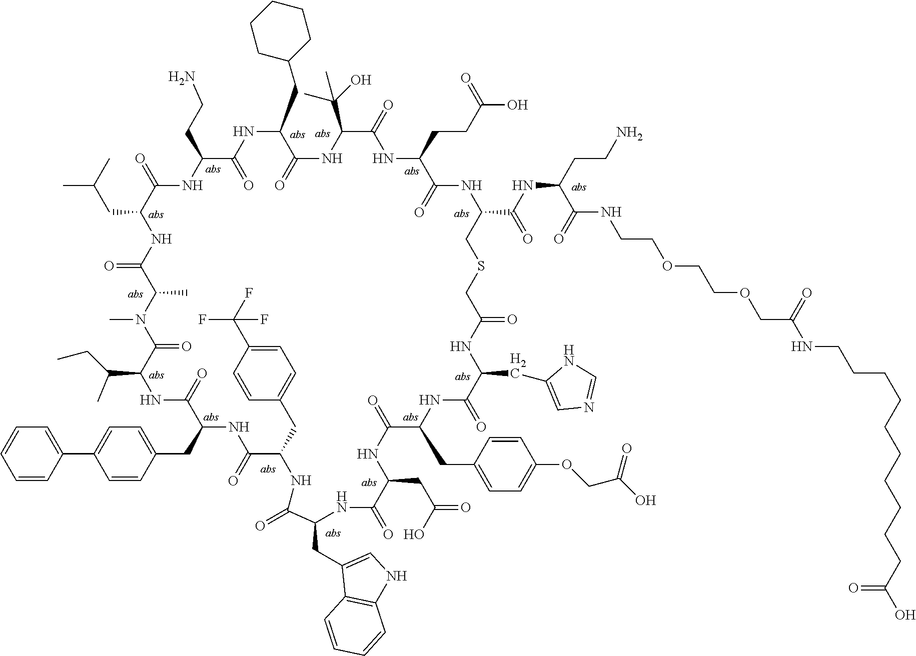 [SMILES: CCC(C)[C@@H]1NC(=O)[C@H](Cc2ccc(-c3ccccc3)cc2)NC(=O)[C@H](Cc2ccc(C(F)(F)F)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2590 was prepared on a 50 μmol scale. The yield of the product was 3.9 mg, and its estimated purity by LCMS analysis was 95.4%. Analysis condition A: Retention time=1.99 min; ESI-MS(+) m/z [M+2H]²⁺: 1249.

### Preparation of Example 2591

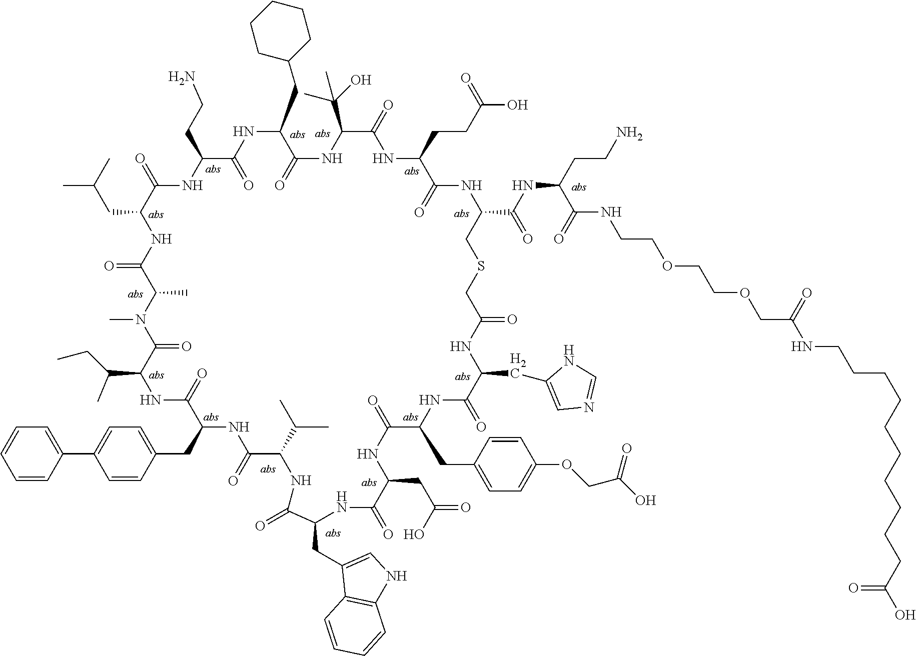 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2591 was prepared on a 50 μmol scale. The yield of the product was 24.1 mg, and its estimated purity by LCMS analysis was 94.3%. Analysis condition A: Retention time=1.74 min; ESI-MS(+) m/z [M+2H]²⁺: 1191.2.

### Preparation of Example 2592

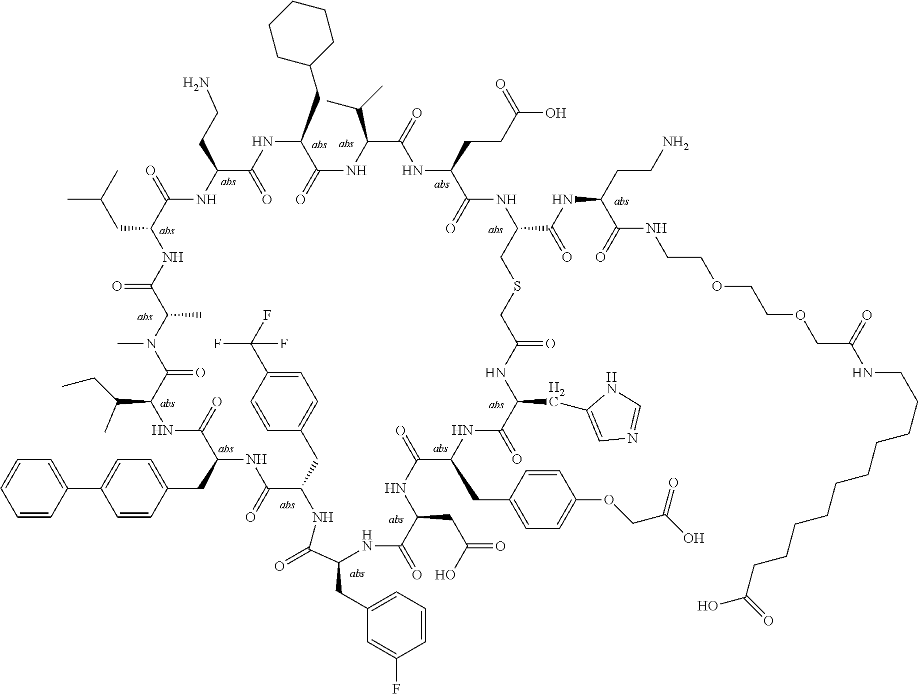 [SMILES: CCC(C)[C@@H]1NC(=O)[C@H](Cc2ccc(-c3ccccc3)cc2)NC(=O)[C@H](Cc2ccc(C(F)(F)F)cc2)NC(=O)[C@H](Cc2cccc(F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C1=O]

Example 2592 was prepared on a 50 μmol scale. The yield of the product was 5.4 mg, and its estimated purity by LCMS analysis was 95.9%. Analysis condition A: Retention time=2.05 min; ESI-MS(+) m/z [M+3H]³⁺: 821.1.

### Preparation of Example 2593

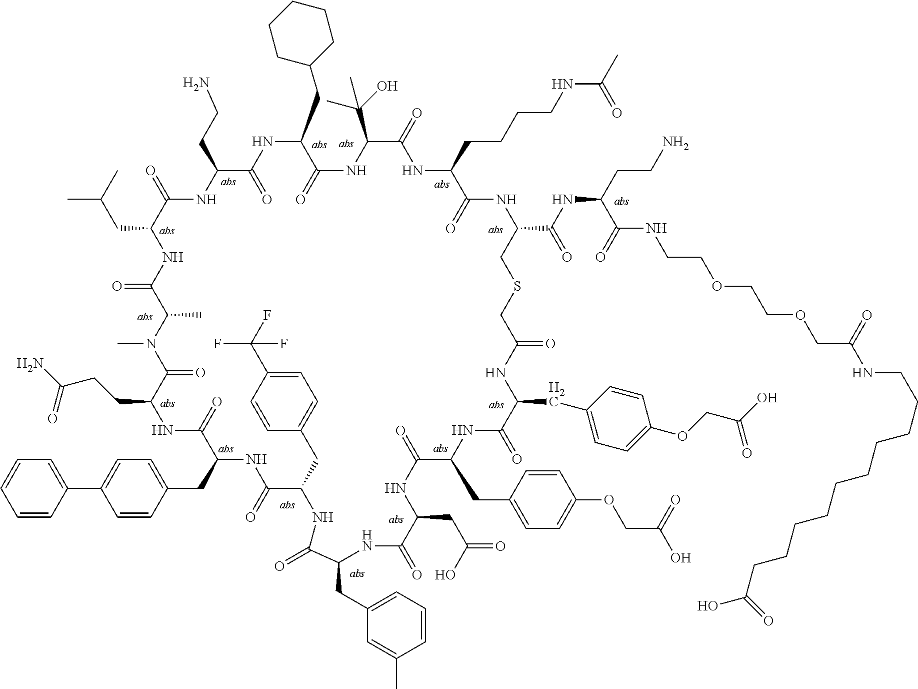 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(F)(F)F)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2593 was prepared on a 50 μmol scale. The yield of the product was 12.7 mg, and its estimated purity by LCMS analysis was 97.5%. Analysis condition B: Retention time=2.11 min; ESI-MS(+) m/z [M+2H]²⁺: 1306.2.

### Preparation of Example 2594

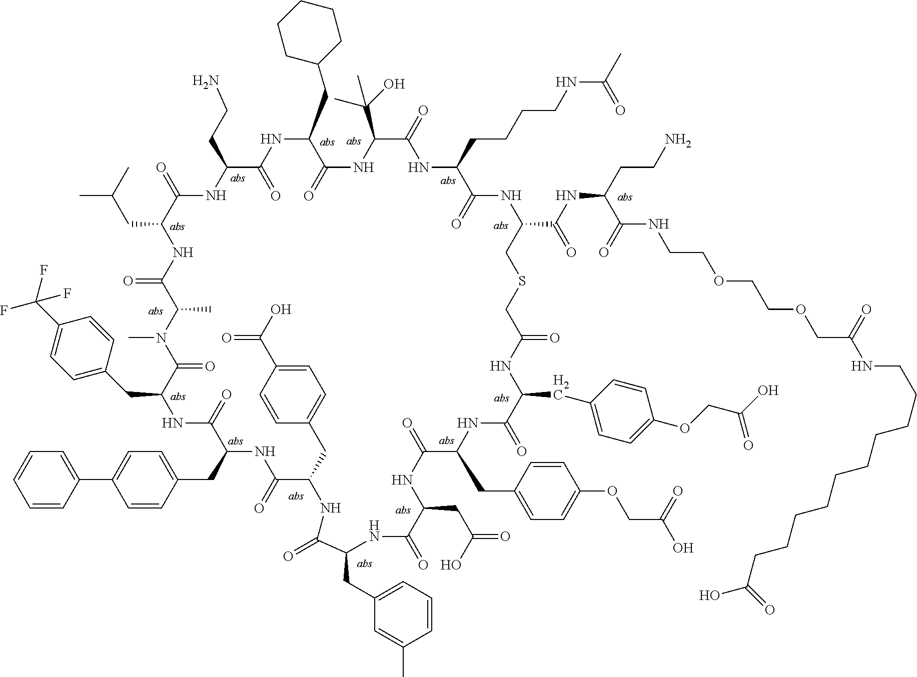 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(F)(F)F)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2594 was prepared on a 50 μmol scale. The yield of the product was 68.8 mg, and its estimated purity by LCMS analysis was 96%. Analysis condition A: Retention time=1.6 min; ESI-MS(+) m/z [M+2H]²⁺: 1338.

### Preparation of Example 2595

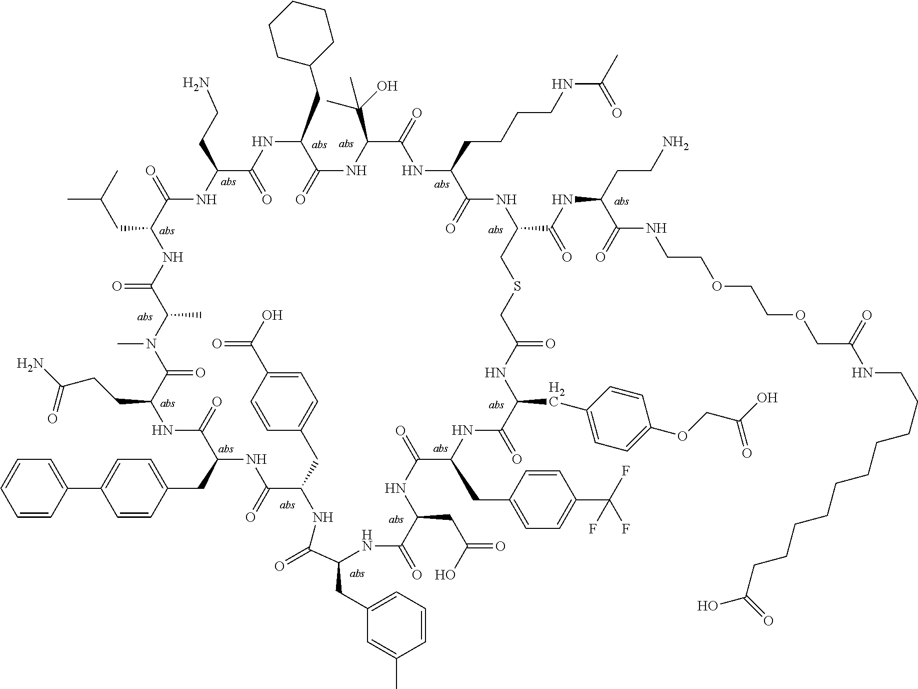 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(C(F)(F)F)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2595 was prepared on a 50 μmol scale. The yield of the product was 47.8 mg, and its estimated purity by LCMS analysis was 89.9%. Analysis condition A: Retention time=1.86 min; ESI-MS(+) m/z [M+2H]²⁺: 1292.

### Preparation of Example 2596

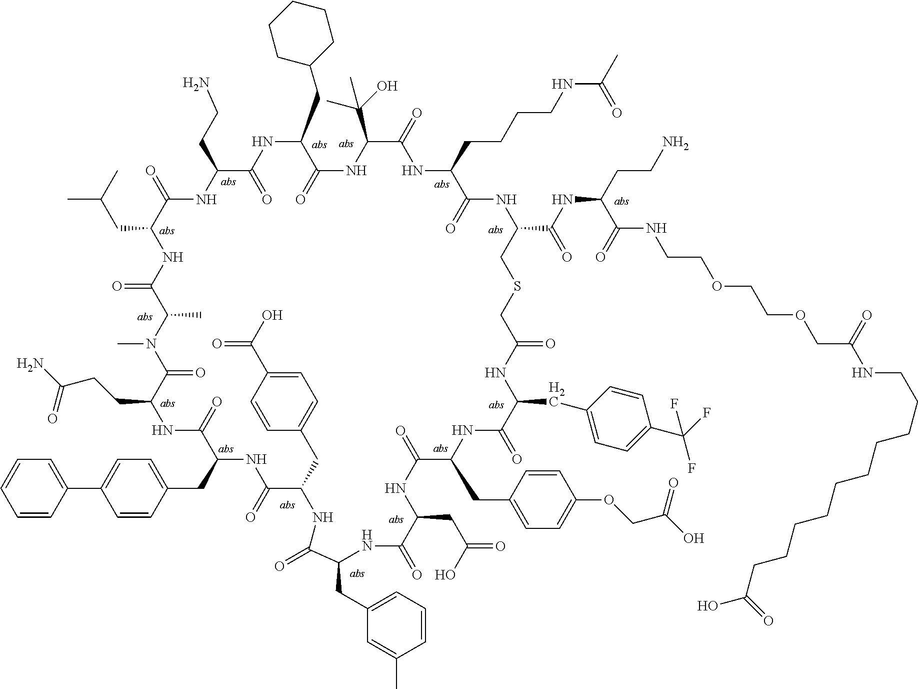 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCOCCOCC(=O)NCCCCCCCCCCC(=O)O)NC1=O]

Example 2596 was prepared on a 50 μmol scale. The yield of the product was 75.6 mg, and its estimated purity by LCMS analysis was 90.3%. Analysis condition A: Retention time=1.63 min; ESI-MS(+) m/z [M+2H]²⁺: 1291.5.

### Preparation of Example 2597

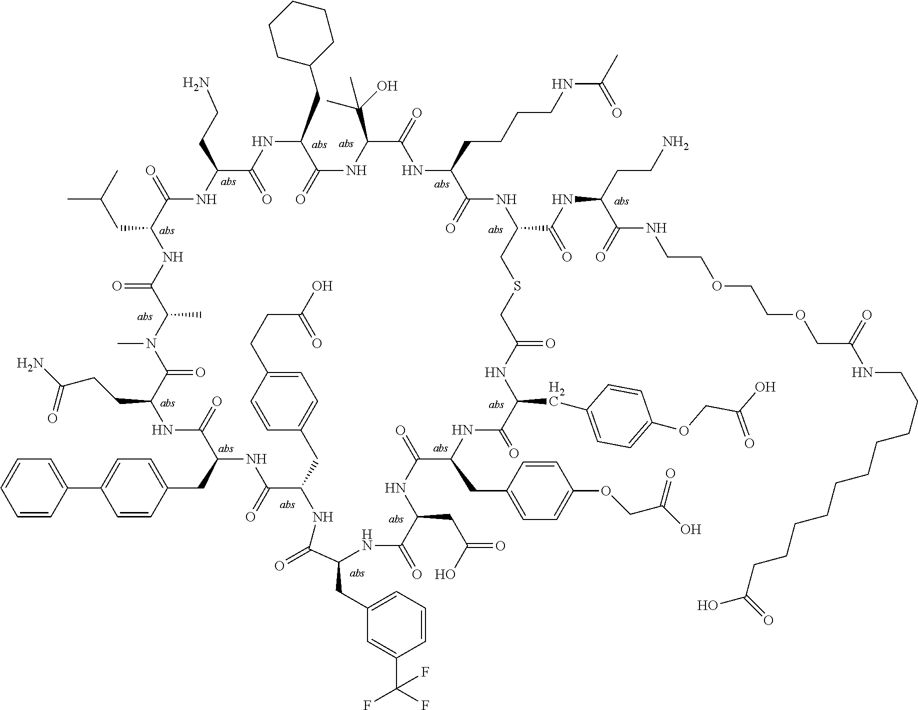 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(CCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2597 was prepared on a 50 μmol scale. The yield of the product was 43.9 mg, and its estimated purity by LCMS analysis was 100%. Analysis condition A: Retention time=1.48 min; ESI-MS(+) m/z [M+2H]²⁺: 1336.4.

### Preparation of Example 2598

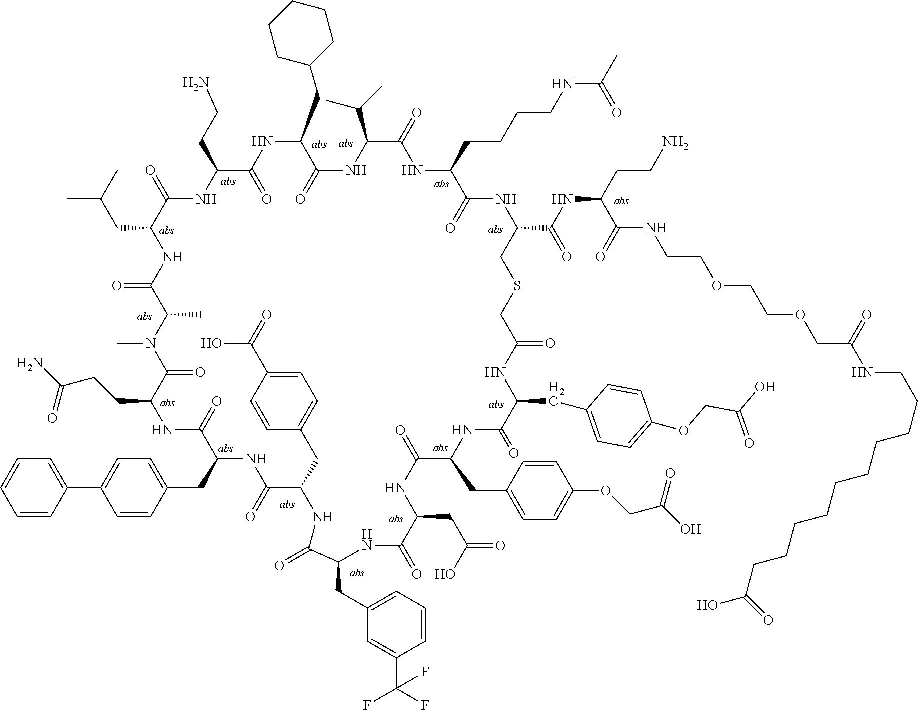 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC1=O]

Example 2598 was prepared on a 50 μmol scale. The yield of the product was 21.1 mg, and its estimated purity by LCMS analysis was 97.9%. Analysis condition A: Retention time=1.47 min; ESI-MS(+) m/z [M+2H]²⁺: 1314.2.

### Preparation of Example 2599

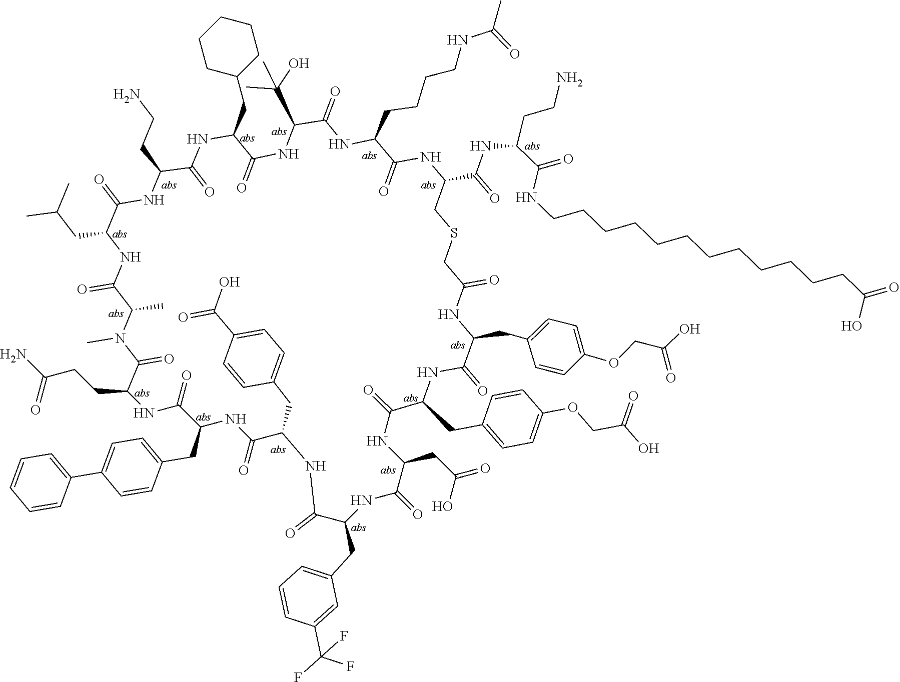 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CCN)C(=O)NCCCCCCCCCCCCC(=O)O)NC1=O]

Example 2599 was prepared on a 50 μmol scale. The yield of the product was 50.4 mg, and its estimated purity by LCMS analysis was 95.3%. Analysis condition A: Retention time=1.59 min; ESI-MS(+) m/z [M+2H]²⁺: 1263.1.

### Preparation of Example 2600

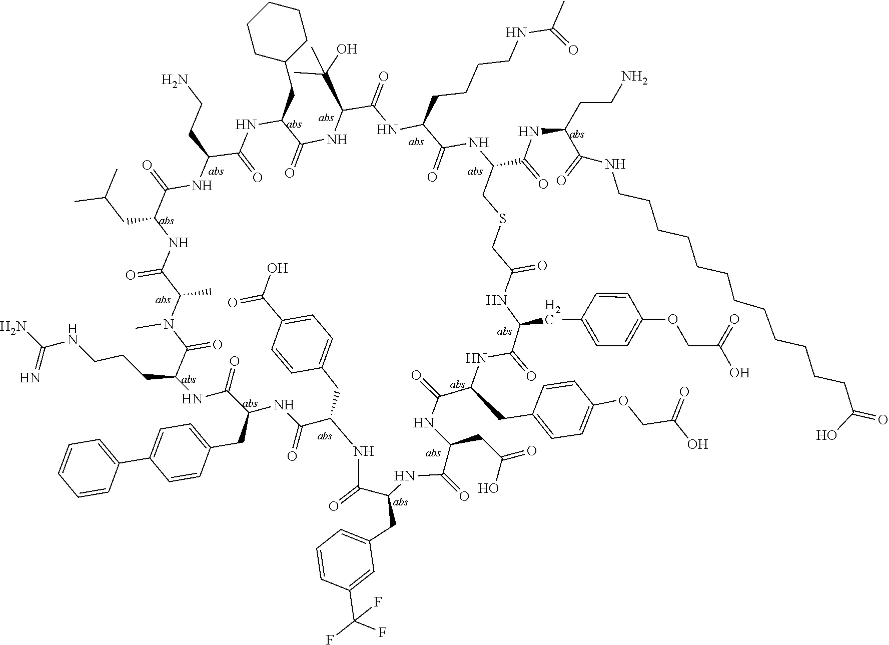 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(=N)N)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2600 was prepared on a 50 μmol scale. The yield of the product was 30.7 mg, and its estimated purity by LCMS analysis was 89%. Analysis condition A: Retention time=1.58 min; ESI-MS(+) m/z [M+2H]²⁺: 1277.5.

### Preparation of Example 2601

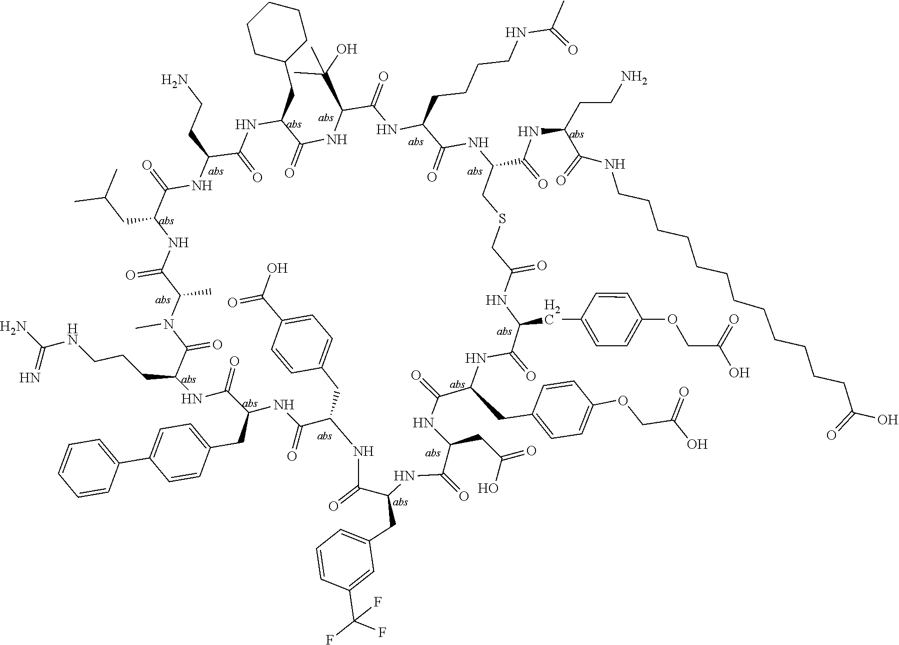 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(=N)N)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2601 was prepared on a 50 μmol scale. The yield of the product was 78.1 mg, and its estimated purity by LCMS analysis was 90.2%. Analysis condition A: Retention time=1.66 min; ESI-MS(+) m/z [M+2H]²⁺: 1292.

### Preparation of Example 2602

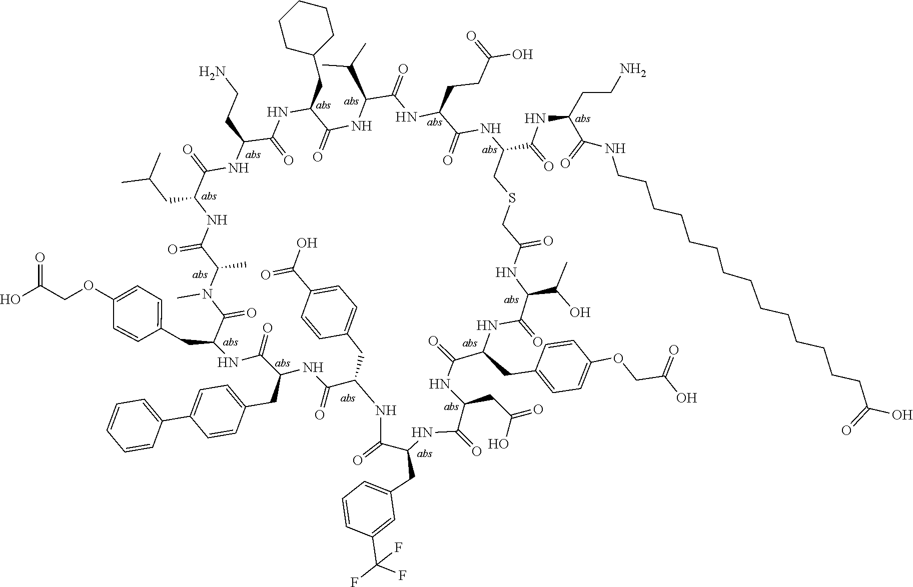 [SMILES: CC(C)C[C@H]1NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2602 was prepared on a 50 μmol scale. The yield of the product was 13.4 mg, and its estimated purity by LCMS analysis was 95%. Analysis condition B: Retention time=1.98 min; ESI-MS(+) m/z [M+2H]²⁺: 1235.

### Preparation of Example 2603

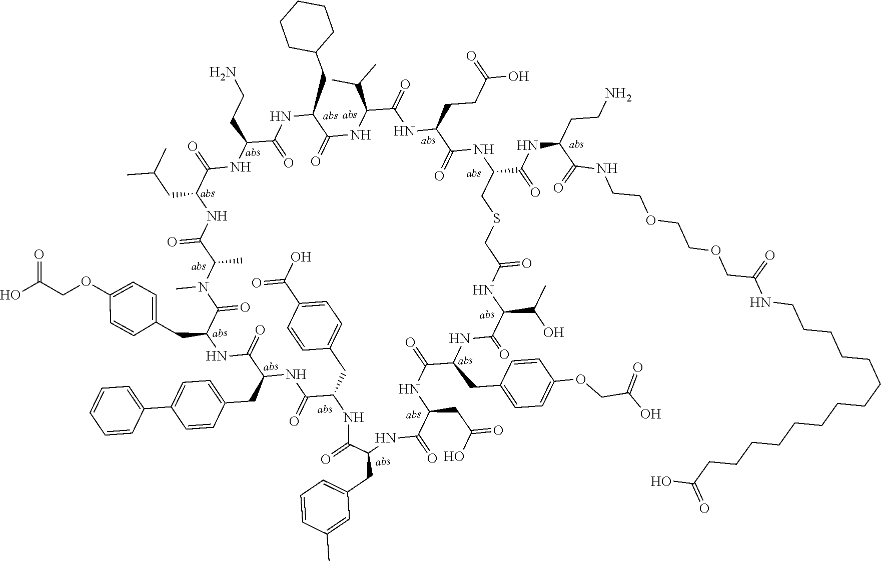 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Example 2603 was prepared on a 50 μmol scale. The yield of the product was 42.1 mg, and its estimated purity by LCMS analysis was 100%. Analysis condition B: Retention time=2.13 min; ESI-MS(+) m/z [M+2H]²⁺: 1280.3.

### Preparation of Example 2604

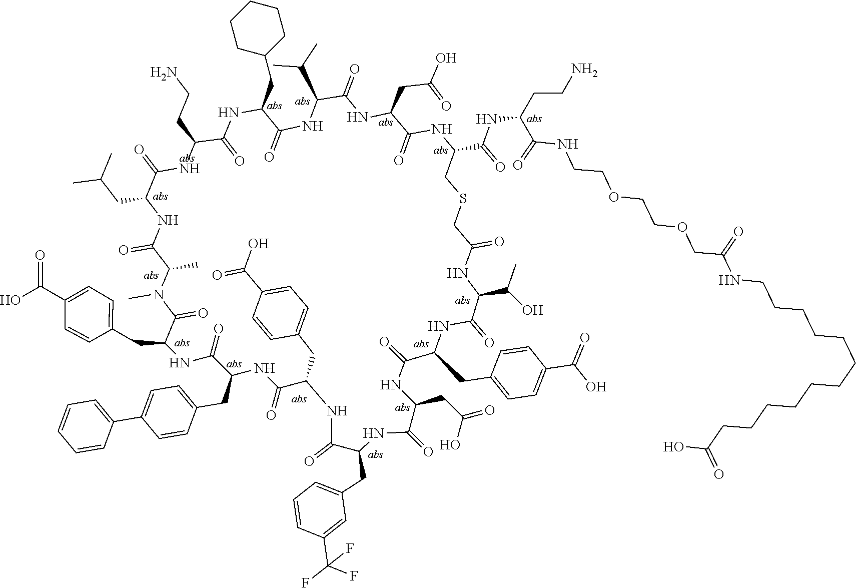 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@H](CCN)C(=O)NCCOCCOCC(=O)NCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Example 2604 was prepared on a 50 μmol scale. The yield of the product was 54.4 mg, and its estimated purity by LCMS analysis was 89.5%. Analysis condition A: Retention time=1.61 min; ESI-MS(+) m/z [M+2H]²⁺: 1256.1.

### Preparation of Example 2605

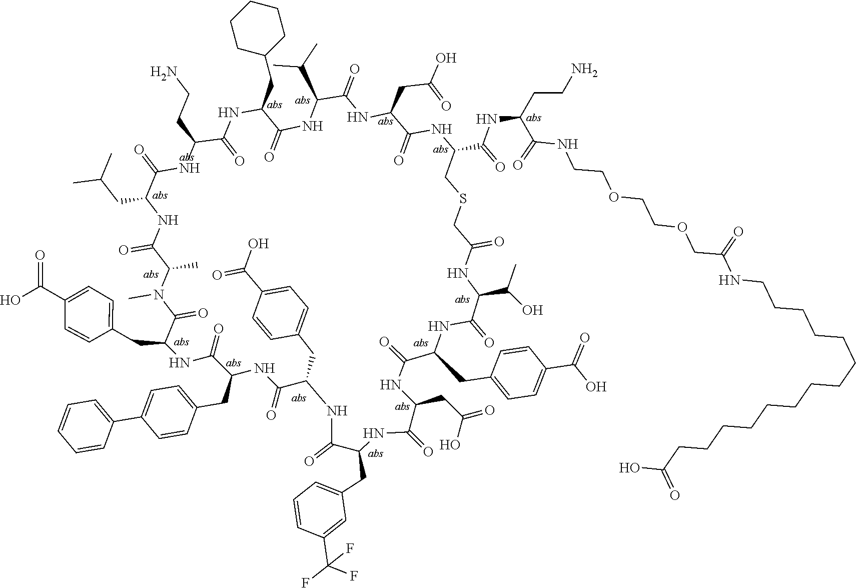 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Example 2605 was prepared on a 50 μmol scale. The yield of the product was 36.9 mg, and its estimated purity by LCMS analysis was 100%. Analysis condition A: Retention time=1.59 min; ESI-MS(+) m/z [M+2H]²⁺: 1270.1.

### Preparation of Example 2606

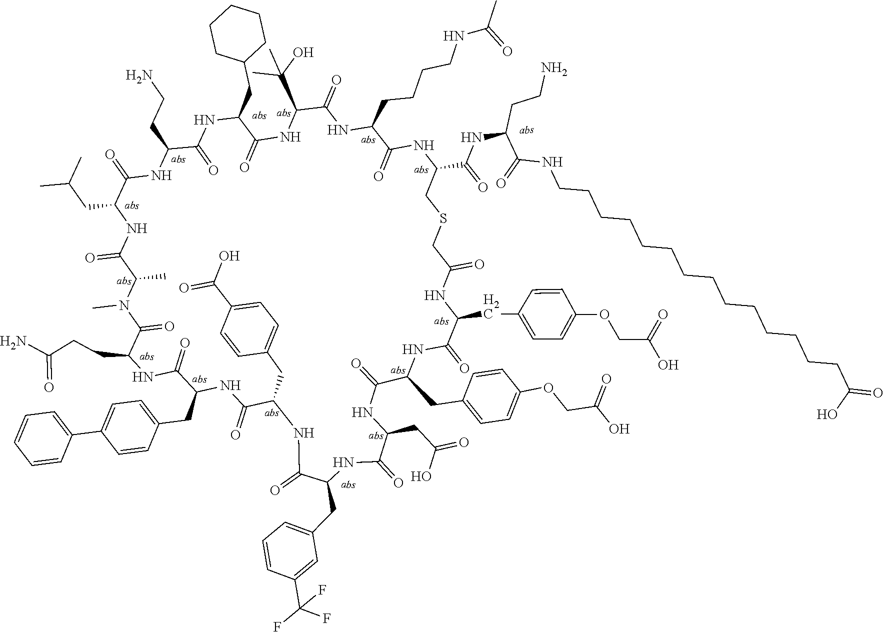 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Example 2606 was prepared on a 50 μmol scale. The yield of the product was 38.9 mg, and its estimated purity by LCMS analysis was 98.5%. Analysis condition A: Retention time=1.67 min; ESI-MS(+) m/z [M+2H]²⁺: 1277.2.

### Preparation of Example 2607

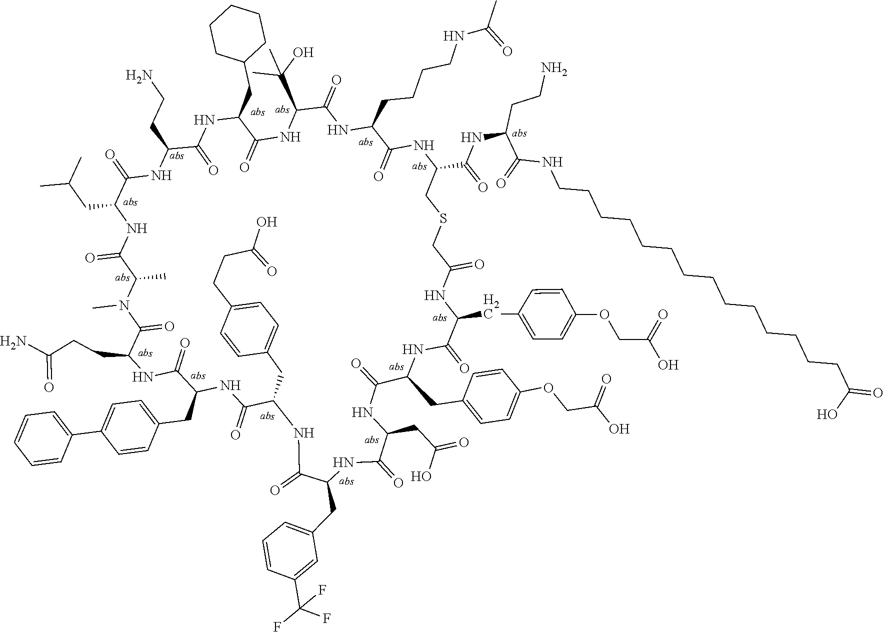 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(CCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Example 2607 was prepared on a 50 μmol scale. The yield of the product was 43.3 mg, and its estimated purity by LCMS analysis was 96.7%. Analysis condition B: Retention time=2.09 min; ESI-MS(+) m/z [M+2H]²⁺: 1292.1.

### Preparation of Example 2608

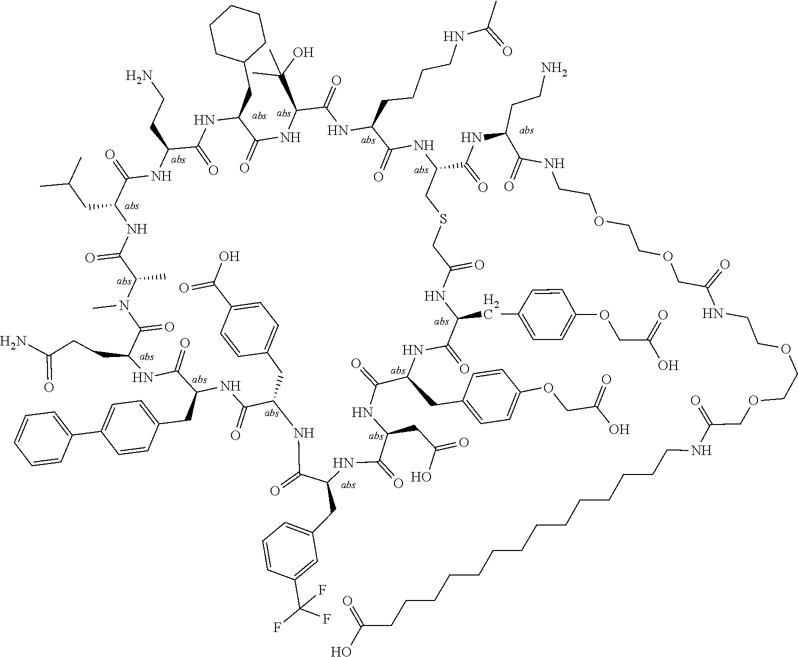 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCCCCCC(=O)O)NC1=O]

Example 2608 was prepared on a 50 μmol scale. The yield of the product was 11.2 mg, and its estimated purity by LCMS analysis was 99.3%. Analysis condition B: Retention time=2.18 min; ESI-MS(+) m/z [M+2H]²⁺: 1422.1.

### Preparation of Example 2609

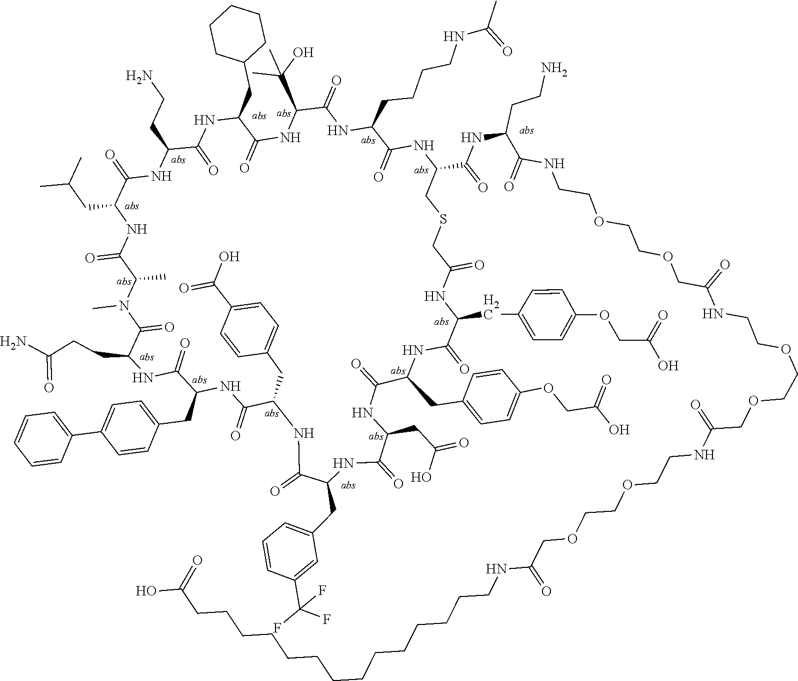 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCOCCOCC(=O)NCCCCCCCCCCCCCCC(=O)O)NC1=O]

Example 2609 was prepared on a 50 μmol scale. The yield of the product was 28.9 mg, and its estimated purity by LCMS analysis was 97.8%. Analysis condition B: Retention time=2.17 min; EST-MS(+) m/z [M+3H]³⁺: 997.

### Preparation of Example 2610

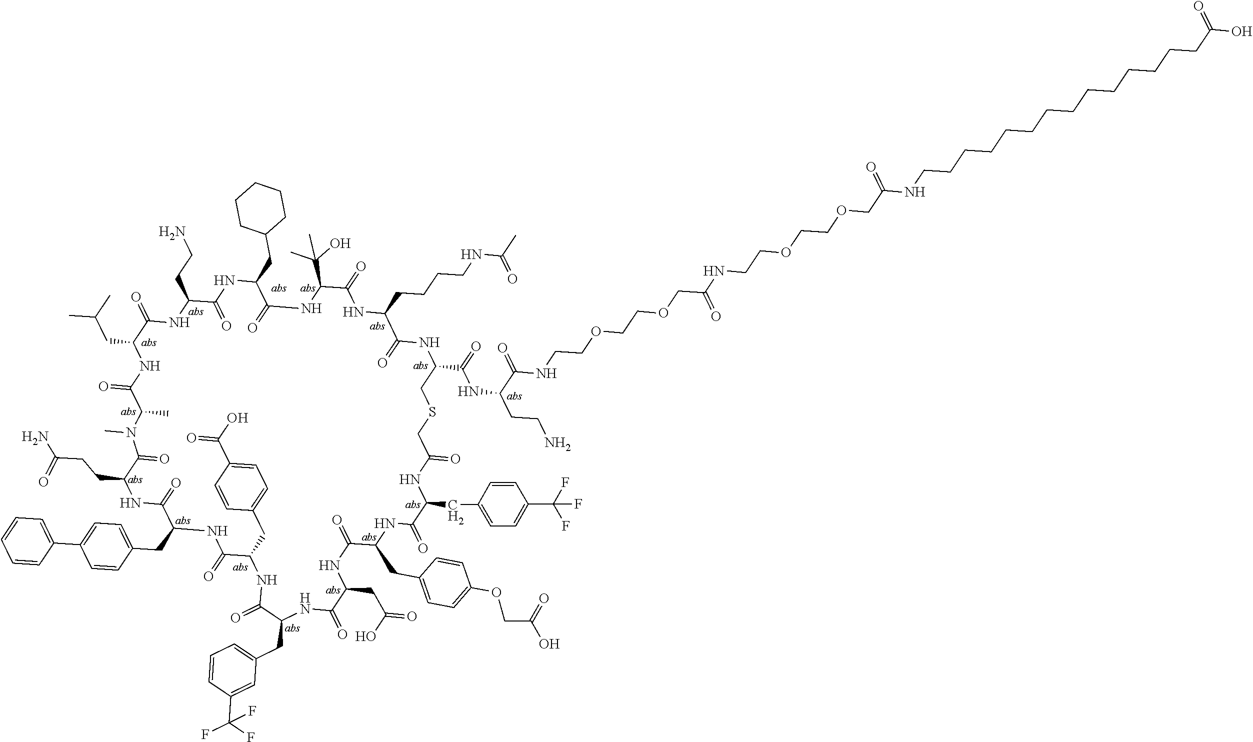 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCOCCOCC(=O)NCCOCCOCC(=O)NCCCCCCCCCCCCCCC(=O)O)NC1=O]

Example 2610 was prepared on a 50 μmol scale. The yield of the product was 29.2 mg, and its estimated purity by LCMS analysis was 98%. Analysis condition A: Retention time=1.92 min; ESI-MS(+) m/z [M+2H]²⁺: 1418.9.

### Preparation of Example 2611

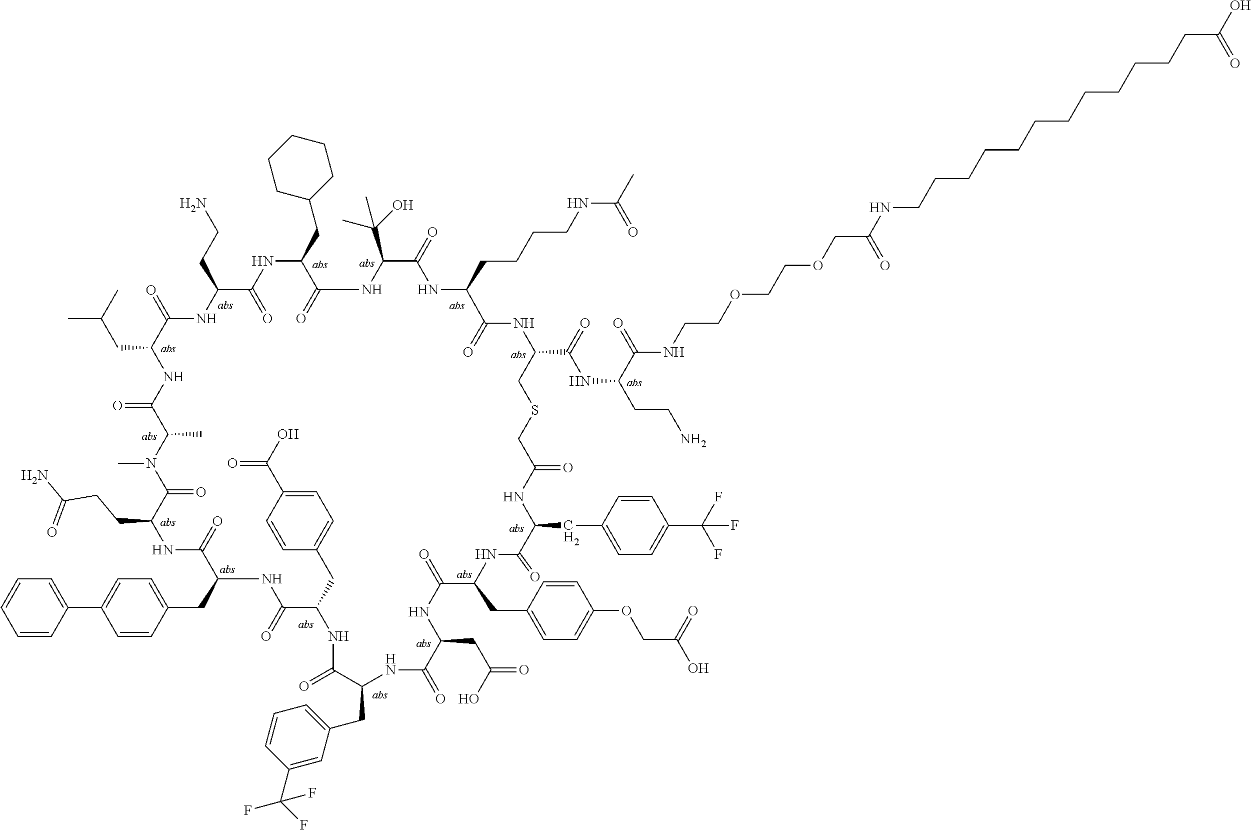 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCOCCOCC(=O)NCCCCCCCCCCCCC(=O)O)NC1=O]

Example 2611 was prepared on a 50 μmol scale. The yield of the product was 34.2 mg, and its estimated purity by LCMS analysis was 96.6%. Analysis condition B: Retention time=2.03 min; ESI-MS(+) m/z [M+2H]²⁺: 1332.3.

### Preparation of Example 2612

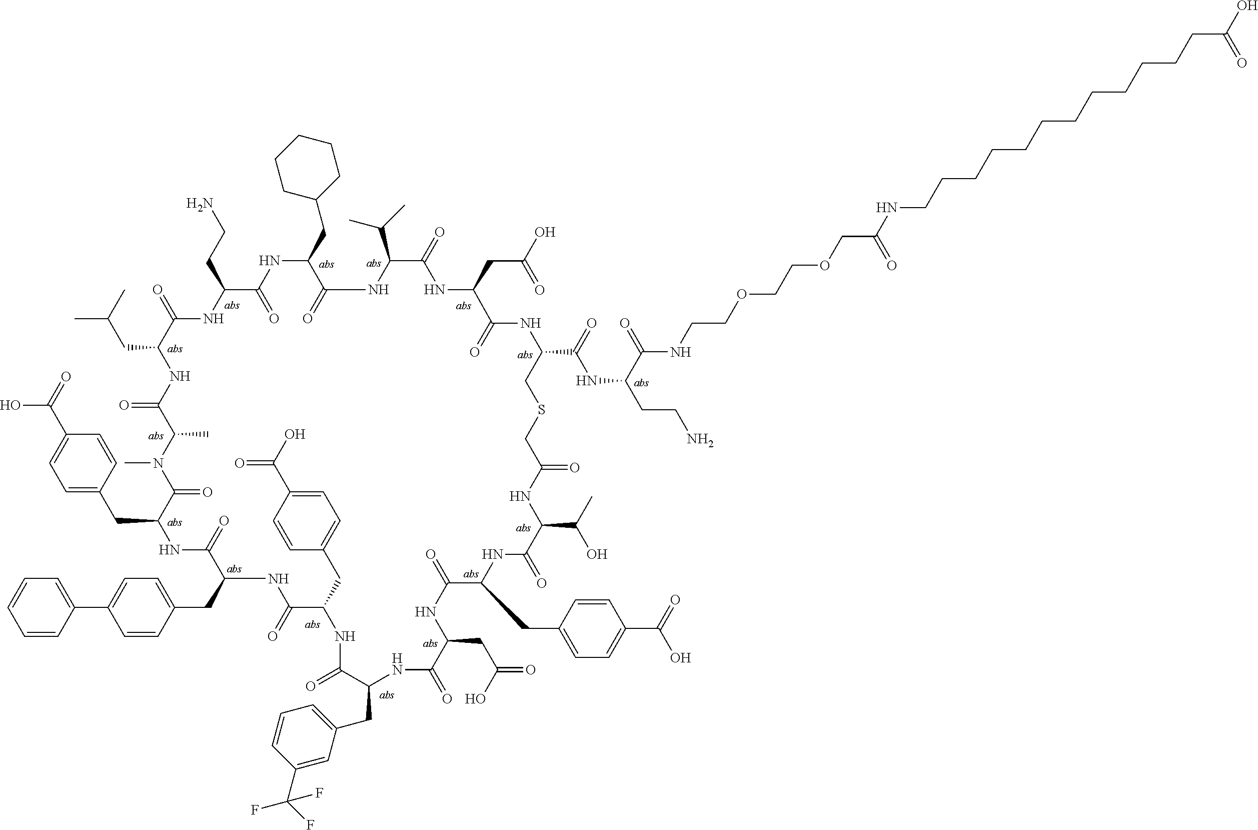 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Example 2612 was prepared on a 50 μmol scale. The yield of the product was 20.5 mg, and its estimated purity by LCMS analysis was 100%. Analysis condition A: Retention time=1.6 min; ESI-MS(+) m/z [M+2H]²⁺: 1256.3.

### Preparation of Example 2613

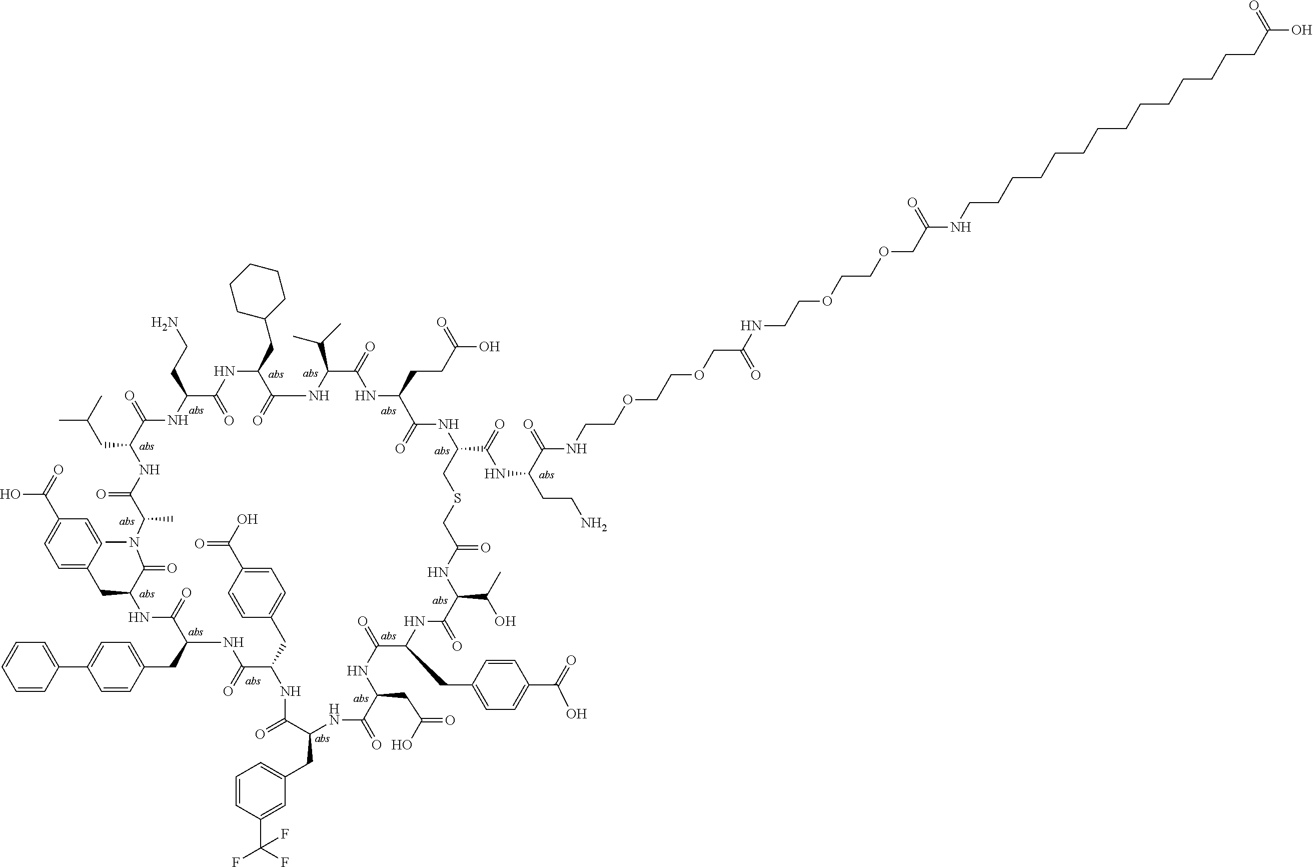 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Example 2613 was prepared on a 50 μmol scale. The yield of the product was 31.7 mg, and its estimated purity by LCMS analysis was 95.4%. Analysis condition A: Retention time=1.61 min; ESI-MS(+) m/z [M+2H]²⁺: 1349.9.

### Preparation of Example 2614

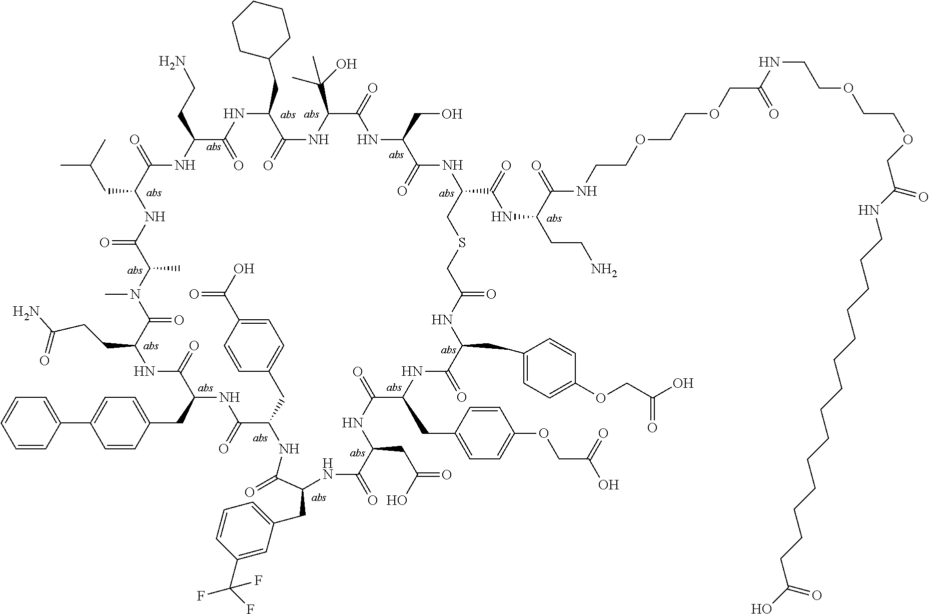 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2614 was prepared on a 50 μmol scale. The yield of the product was 17.5 mg, and its estimated purity by LCMS analysis was 94.9%. Analysis condition A: Retention time=1.76 min; ESI-MS(+) m/z [M+2H]²⁺: 1381.

### Preparation of Example 2615

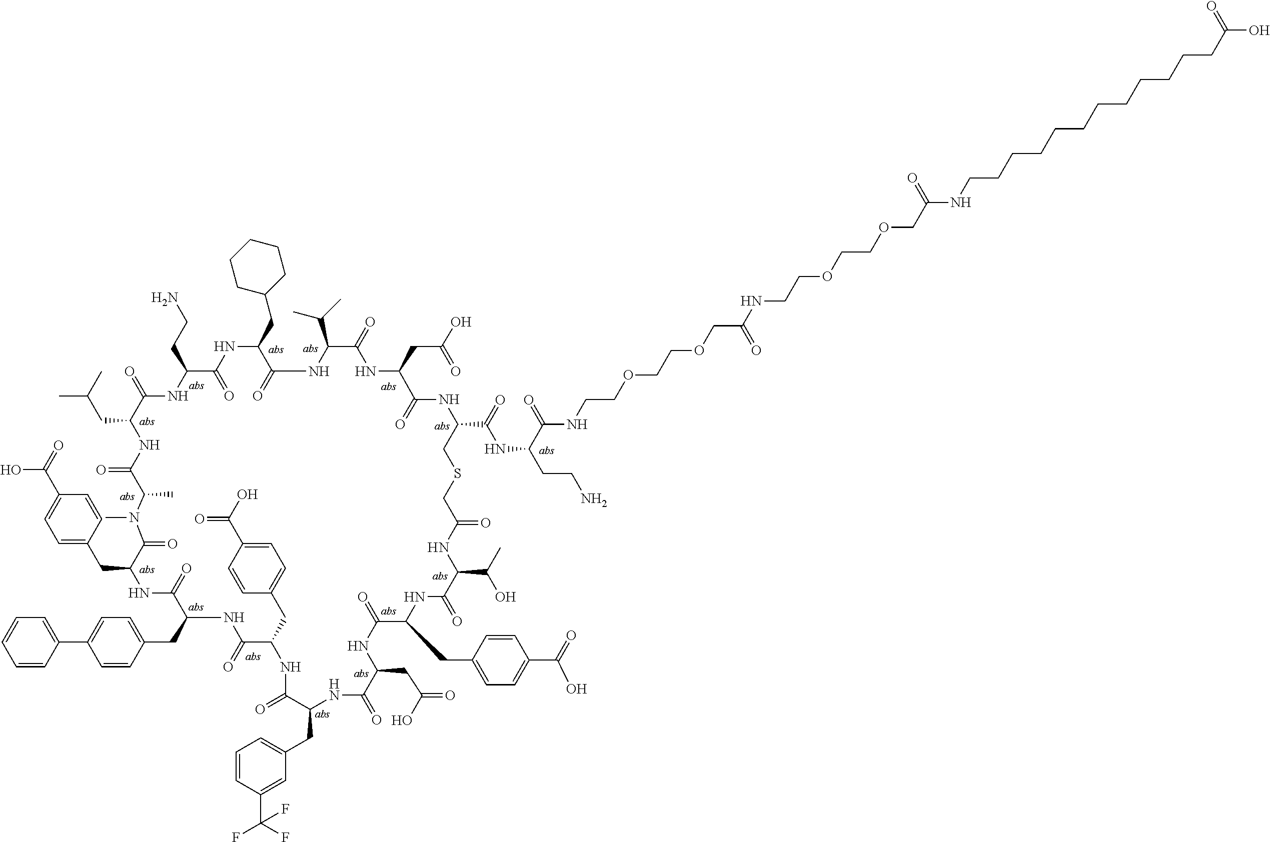 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Example 2615 was prepared on a 50 μmol scale. The yield of the product was 48.3 mg, and its estimated purity by LCMS analysis was 93%. Analysis condition B: Retention time=1.93 min; ESI-MS(+) m/z [M+2H]²⁺: 1329.1.

### Preparation of Example 2616

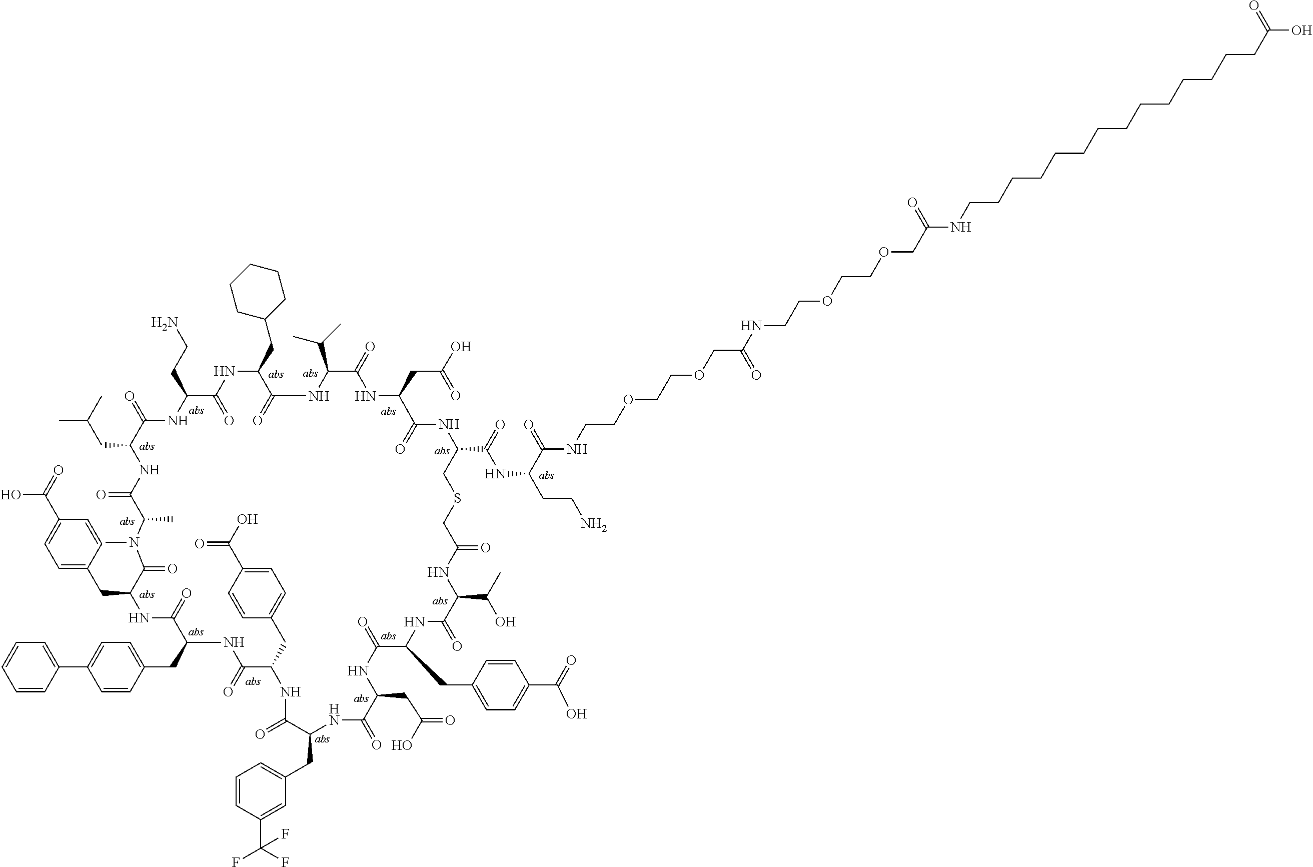 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Example 2616 was prepared on a 50 μmol scale. The yield of the product was 30.4 mg, and its estimated purity by LCMS analysis was 95.8%. Analysis condition A: Retention time=1.71 min; ESI-MS(+) m/z [M+2H]²⁺: 1343.

### Preparation of Example 2617

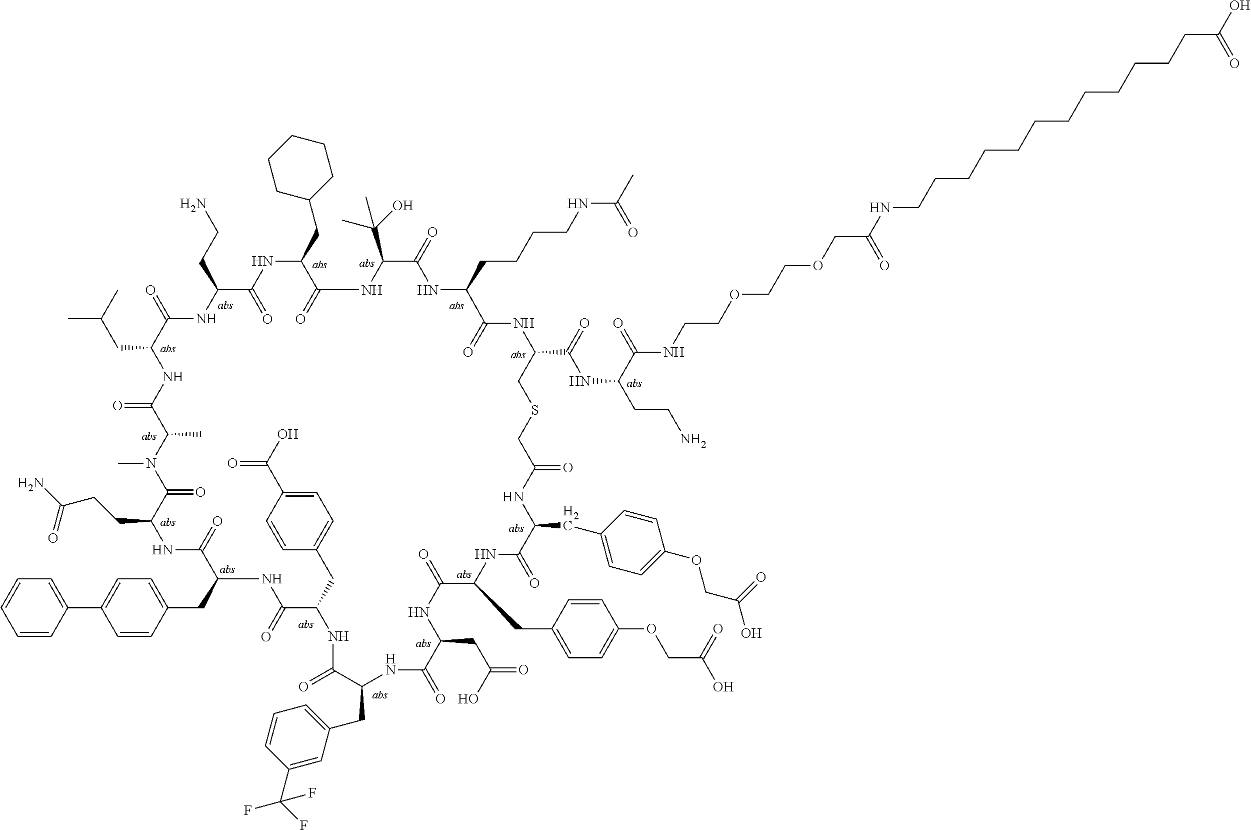 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCC(=O)O)NC1=O]

Example 2617 was prepared on a 50 μmol scale. The yield of the product was 38.2 mg, and its estimated purity by LCMS analysis was 100.%. Analysis condition A: Retention time=1.61 min: ESI-MS(+) m/z [M+2H]²⁺: 1336.1.

### Preparation of Example 2618

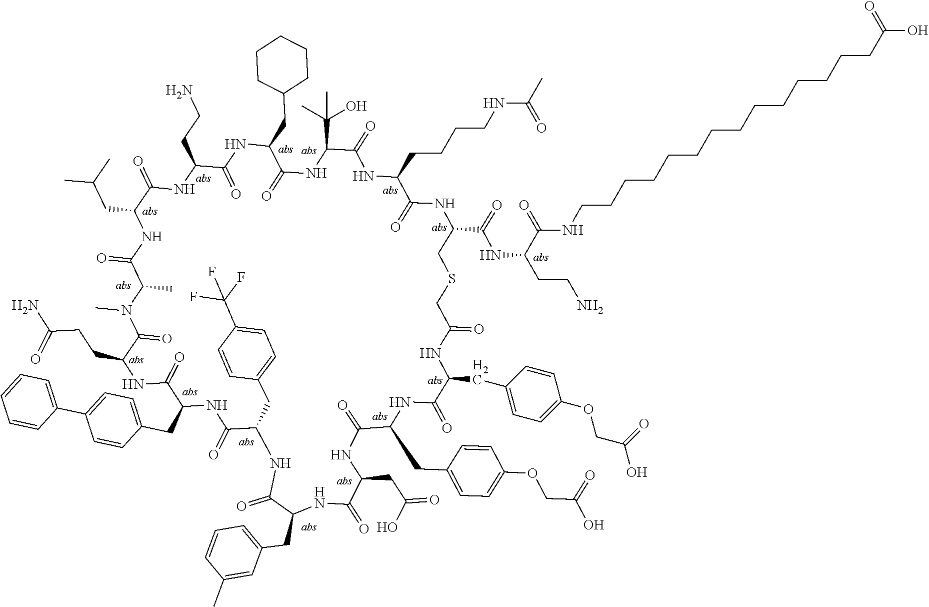 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(F)(F)F)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Example 2618 was prepared on a 50 μmol scale. The yield of the product was 27.2 mg, and its estimated purity by LCMS analysis was 94%. Analysis condition B: Retention time=2.14 min; ESI-MS(+) m/z [M+2H]²⁺: 1262.3.

### Preparation of Example 2619

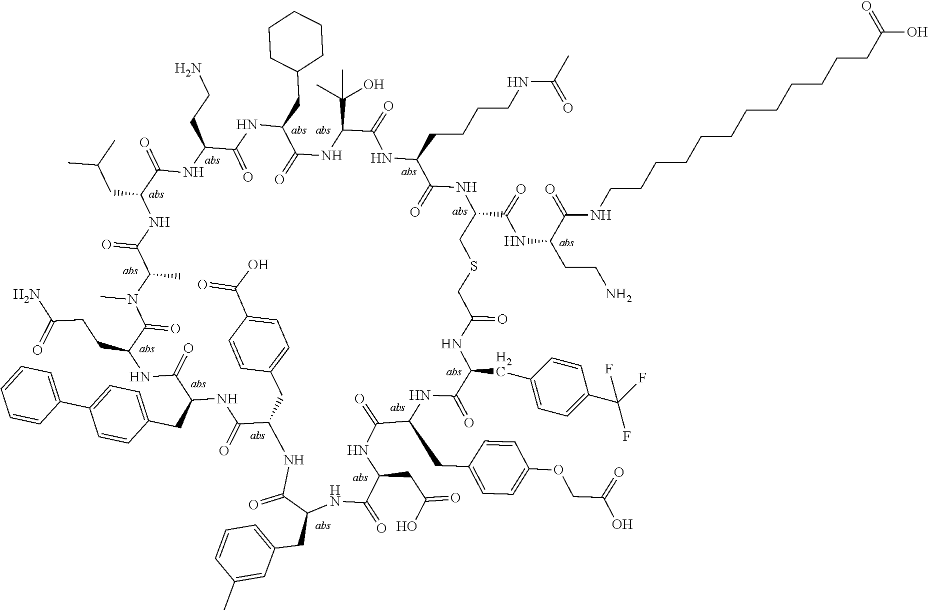 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCCCCCCCCCCCC(=O)O)NC1=O]

Example 2619 was prepared on a 50 μmol scale. The yield of the product was 54.5 mg, and its estimated purity by LCMS analysis was 92.2%. Analysis condition A: Retention time=1.69 min; ESI-MS(+) m/z [M+2H]²⁺: 1233.1.

### Preparation of Example 2620

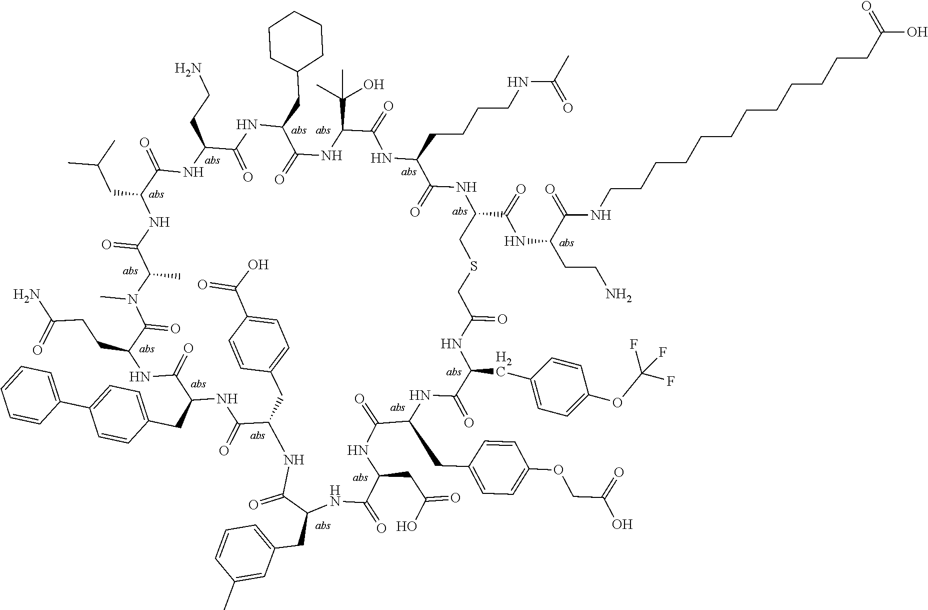 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OC(F)(F)F)cc2)NC(=O)CSC[C@@H](C(=O)N[C@@H](CCN)C(=O)NCCCCCCCCCCCCC(=O)O)NC1=O]

Example 2620 was prepared on a 50 μmol scale. The yield of the product was 60.9 mg, and its estimated purity by LCMS analysis was 92.8%. Analysis condition A: Retention time=1.78 min; ESI-MS(+) m/z [M+2H]²⁺: 1241.1.

### Preparation of Example 2621

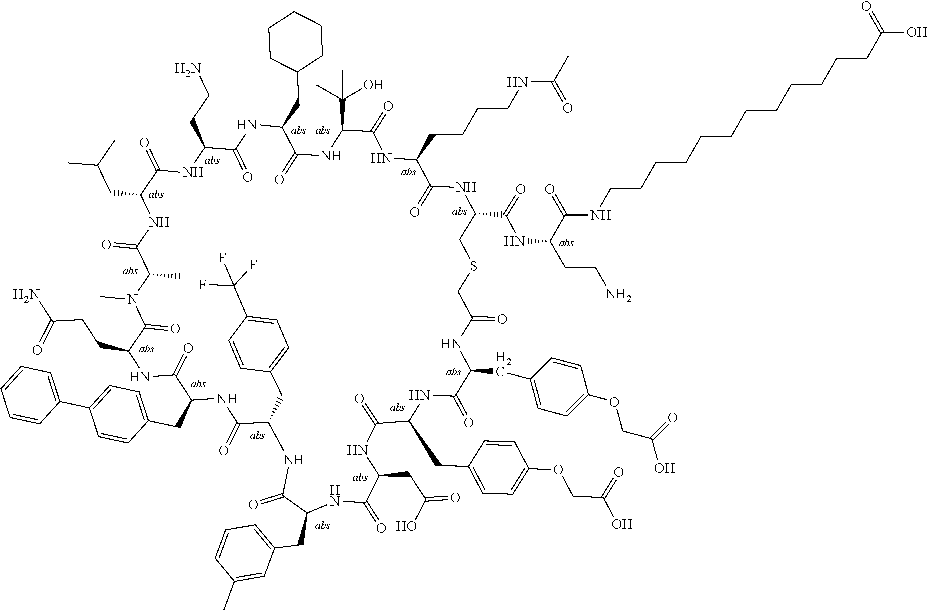 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(F)(F)F)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2621 was prepared on a 50 μmol scale. The yield of the product was 25.5 mg, and its estimated purity by LCMS analysis was 94.9%. Analysis condition A: Retention time=1.9 min; ESI-MS(+) m/z [M+2H]²⁺: 1248.1.

### Preparation of Example 2622

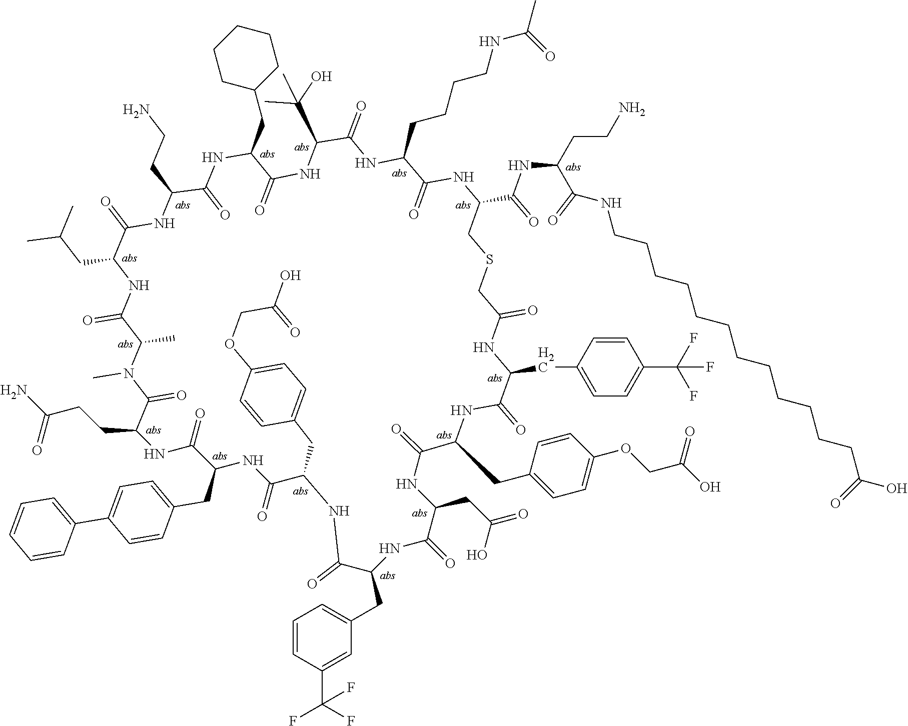 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCCCCCCCCCCCC(=O)O)NC1=O]

Example 2622 was prepared on a 50 μmol scale. The yield of the product was 19.9 mg, and its estimated purity by LCMS analysis was 98.8%. Analysis condition A: Retention time=1.87 min; ESI-MS(+) m/z [M+2H]²⁺: 1275.1.

### Preparation of Example 2623

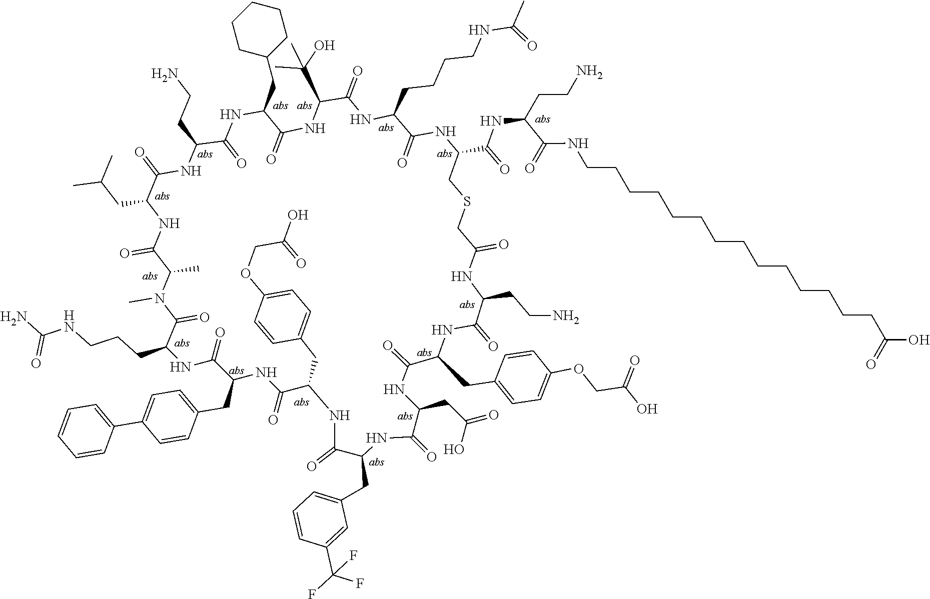 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NCCCCCCCCCCCCCCC(=O)O)NC1=O]

Example 2623 was prepared on a 50 μmol scale. The yield of the product was 18.1 mg, and its estimated purity by LCMS analysis was 100%. Analysis condition B: Retention time=1.9 min; ESI-MS(+) m/z [M+2H]²⁺: 1246.2.

### Preparation of Example 2624

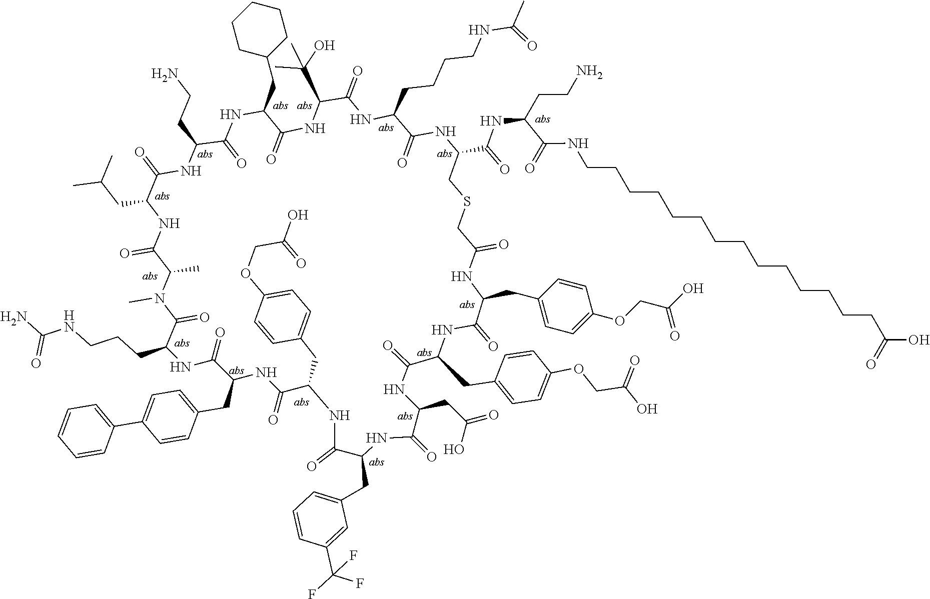 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Example 2624 was prepared on a 50 μmol scale. The yield of the product was 15.8 mg, and its estimated purity by L CMS analysis was 100%. Analysis condition B: Retention time=2.01 min; ESI-MS(+) m/z [M+2H]²⁺: 1307.

### Preparation of Example 2625

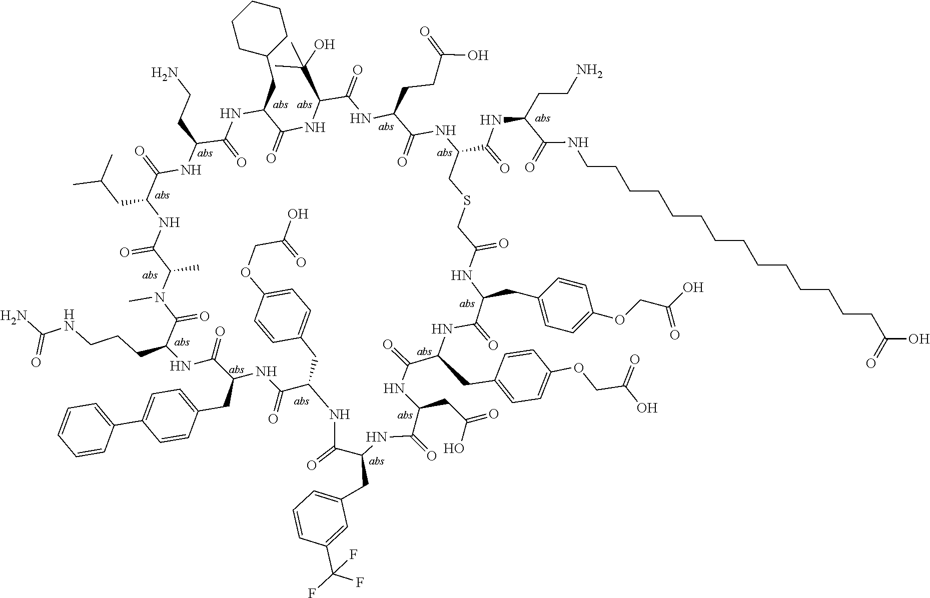 [SMILES: CC(C)C[C@H]1NC(=O)[C@H](C)N(C)C(=O)[C@H](CCCNC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2625 was prepared on a 50 μmol scale. The yield of the product was 21.9 mg, and its estimated purity by LCMS analysis was 95.3%. Analysis condition A: Retention time=1.56 min; ESI-MS(+) m/z [M+2H]²⁺: 1286.2.

### Preparation of Example 2626

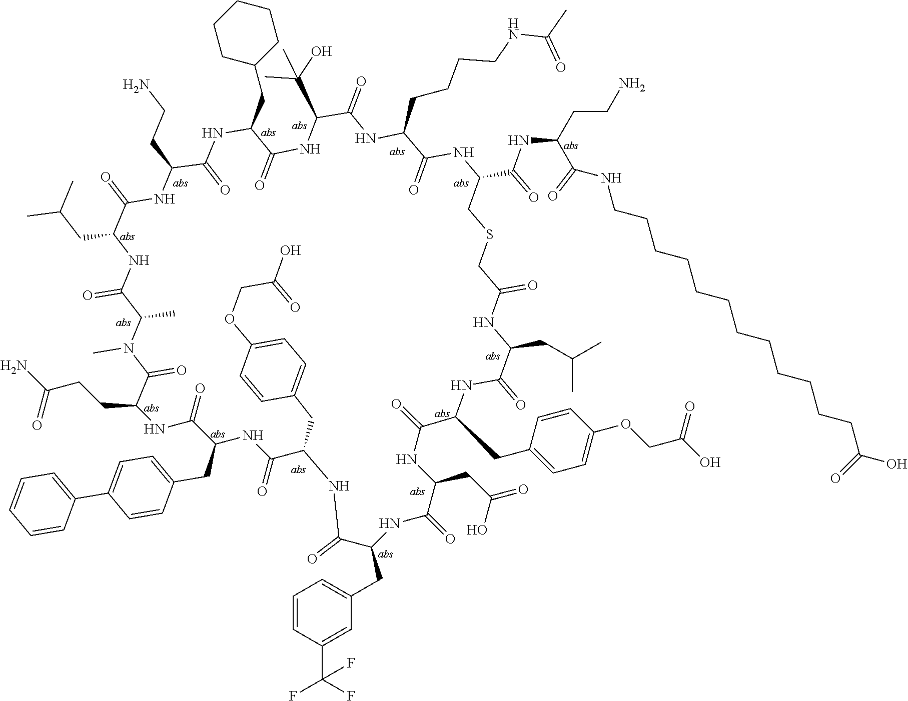 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC(C)C)NC(=O)CSC[C@@H](C(=O)N[C@@H](CCN)C(=O)NCCCCCCCCCCCCC(=O)O)NC1=O]

Example 2626 was prepared on a 50 μmol scale. The yield of the product was 19.8 mg, and its estimated purity by LCMS analysis was 99.5%. Analysis condition B: Retention time=2.01 min; ESI-MS(+) m/z [M+2H]²⁺: 1224.1.

### Preparation of Example 2627

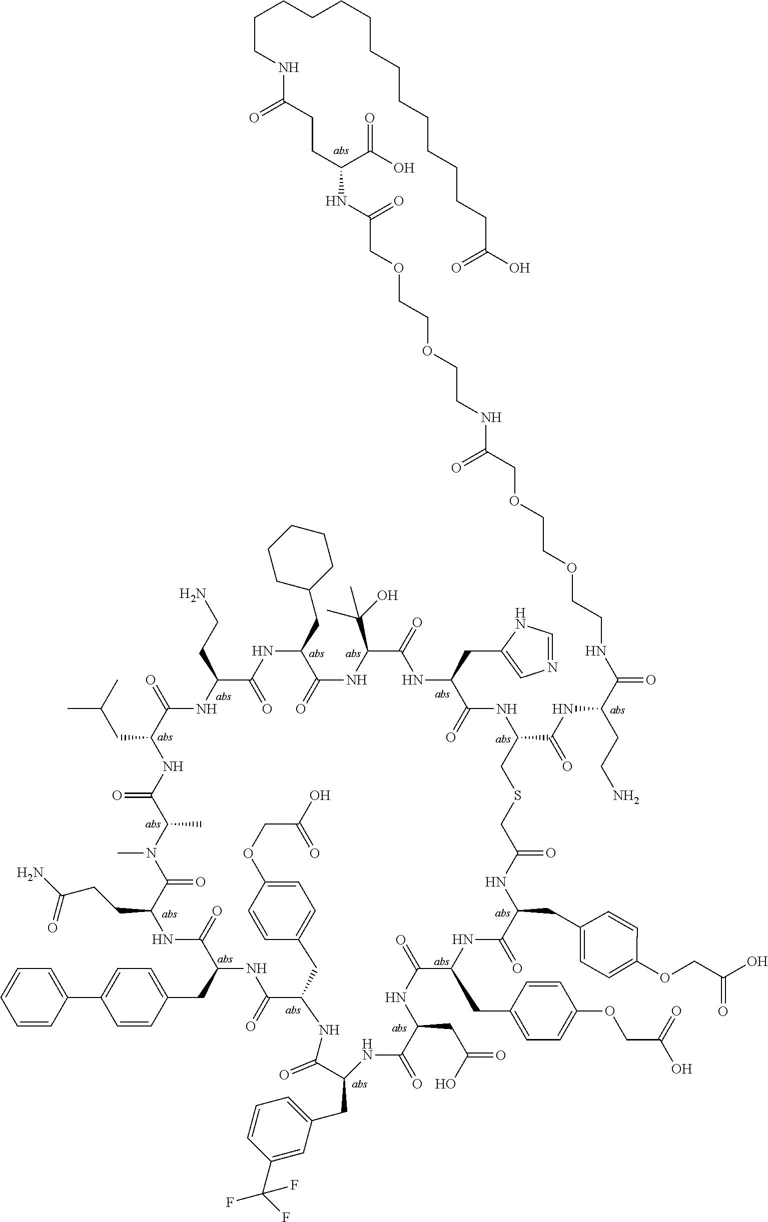 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)N[C@H](CCC(=O)NCCCCCCCCCCCCCCC(=O)O)C(=O)O)NC(=O)[C@H](Cc2cnc[nH]2)NC(=O)[C@H](C(C)(C)O)NC(=O)[C@H](CC2CCCCC2)NC(=O)[C@H](CCN)NC1=O]

Example 2627 was prepared on a 50 μmol scale. The yield of the product was 26.1 mg, and its estimated purity by LCMS analysis was 100%. Analysis condition A: Retention time=1.66 min; ESI-MS(+) m/z [M+2H]²⁺: 1485.4.

### Preparation of Example 2628

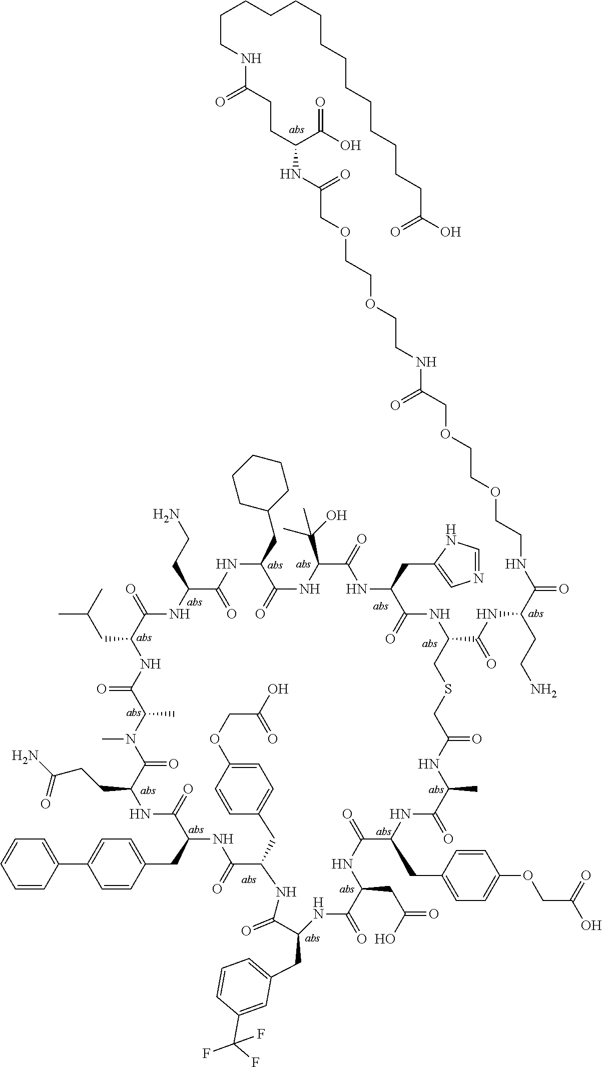 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCC(=O)NCCOCCOCC(=O)N[C@H](CCC(=O)NCCCCCCCCCCCCCCC(=O)O)C(=O)O)NC(=O)[C@H](Cc2cnc[nH]2)NC(=O)[C@H](C(C)(C)O)NC(=O)[C@H](CC2CCCCC2)NC(=O)[C@H](CCN)NC1=O]

Example 2628 was prepared on a 50 μmol scale. The yield of the product was 8.5 mg, and its estimated purity by LCMS analysis was 100%. Analysis condition B: Retention time=1.99 min; ESI-MS(+) m/z [M+2H]²⁺: 1410.

### Preparation of Example 2629

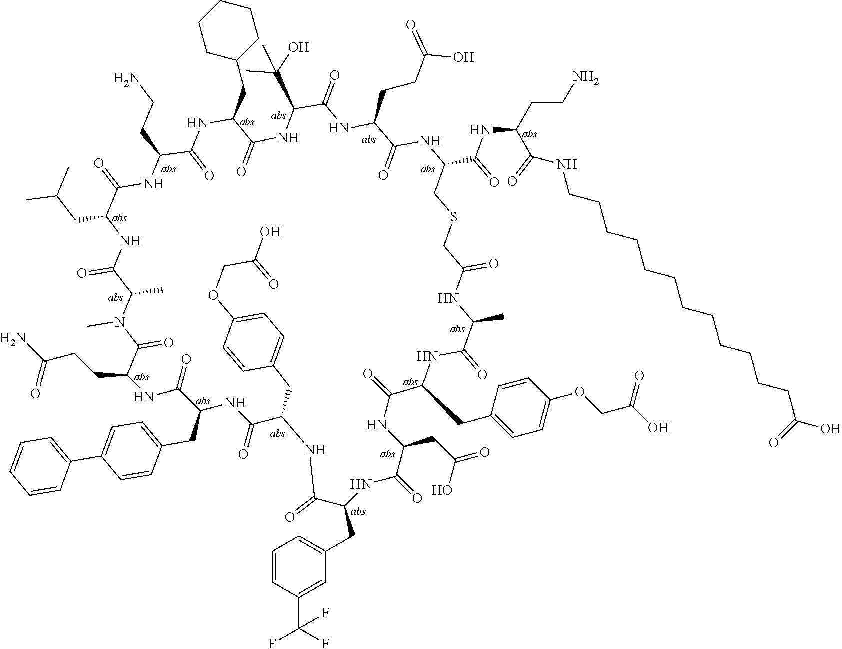 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2629 was prepared on a 50 μmol scale. The yield of the product was 25.9 mg, and its estimated purity by LCMS analysis was 100%. Analysis condition A: Retention time=1.65 min; ESI-MS(+) m/z [M+2H]²⁺: 1183.4.

### Preparation of Example 2630

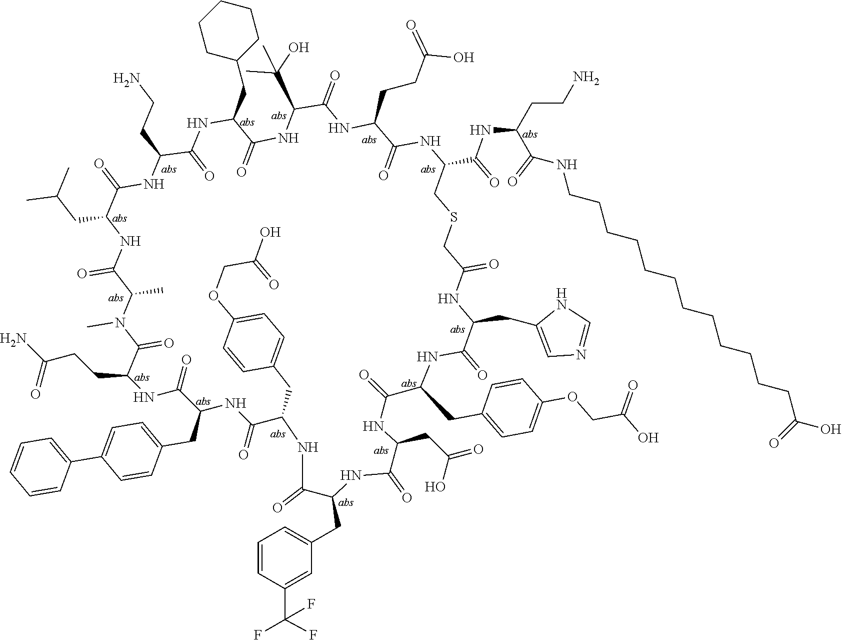 [SMILES: CC(C)C[C@H]1NC(=O)[C@H](C)N(C)C(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Example 2630 was prepared on a 50 μmol scale. The yield of the product was 45 mg, and its estimated purity by LCMS analysis was 99.500. Analysis condition B: Retention time=1.78 min; ESI-MS(+) m/z [M+2H]²⁺: 1215.3.

### Preparation of Example 2631

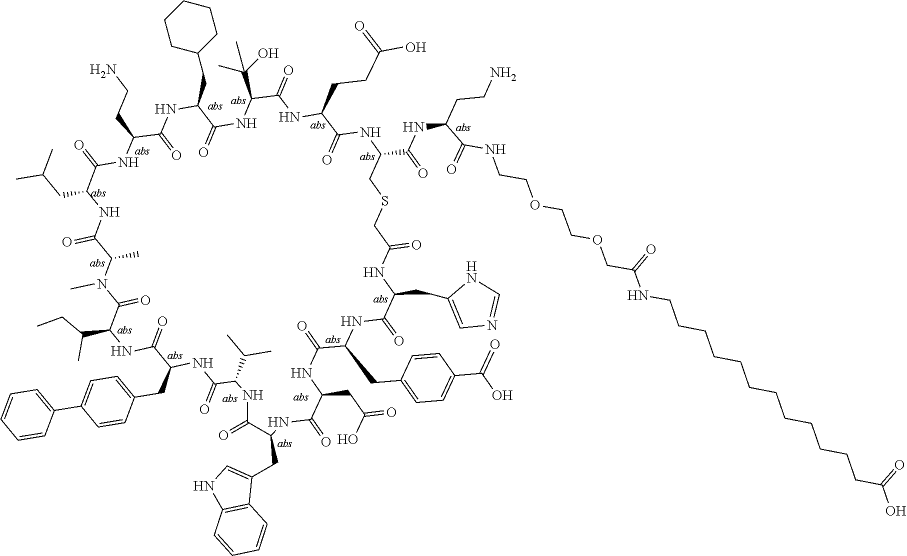 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2631 was prepared on a 50 μmol scale. The yield of the product was 7.1 mg, and its estimated purity by LCMS analysis was 96.7%. Analysis condition A: Retention time=1.72 min; ESI-MS(+) m/z [M+2H]²⁺: 1190.1.

### Preparation of Example 2632

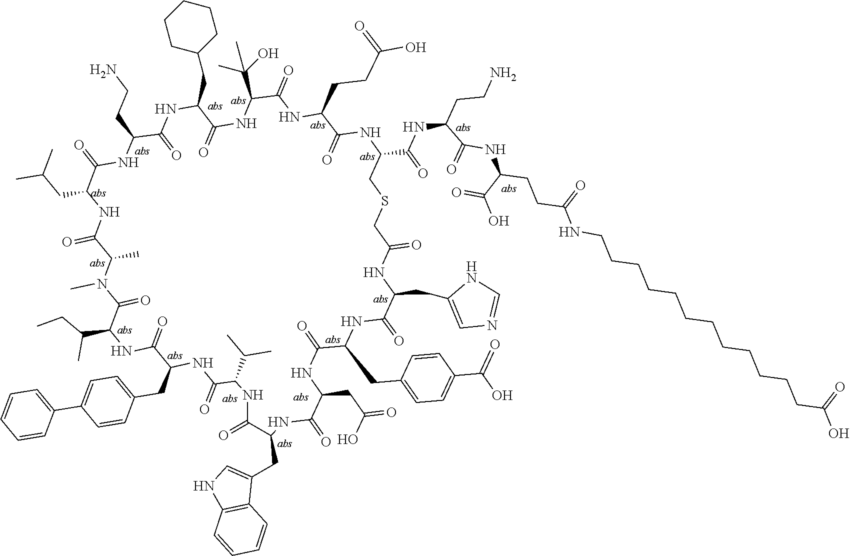 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)N[C@@H](CCC(=O)NCCCCCCCCCCCCC(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2632 was prepared on a 50 μmol scale. The yield of the product was 11.5 mg, and its estimated purity by LCMS analysis was 97.6%. Analysis condition B: Retention time=1.82 min; ESI-MS(+) m/z [M+2H]²⁺: 1182.1.

### Preparation of Example 2633

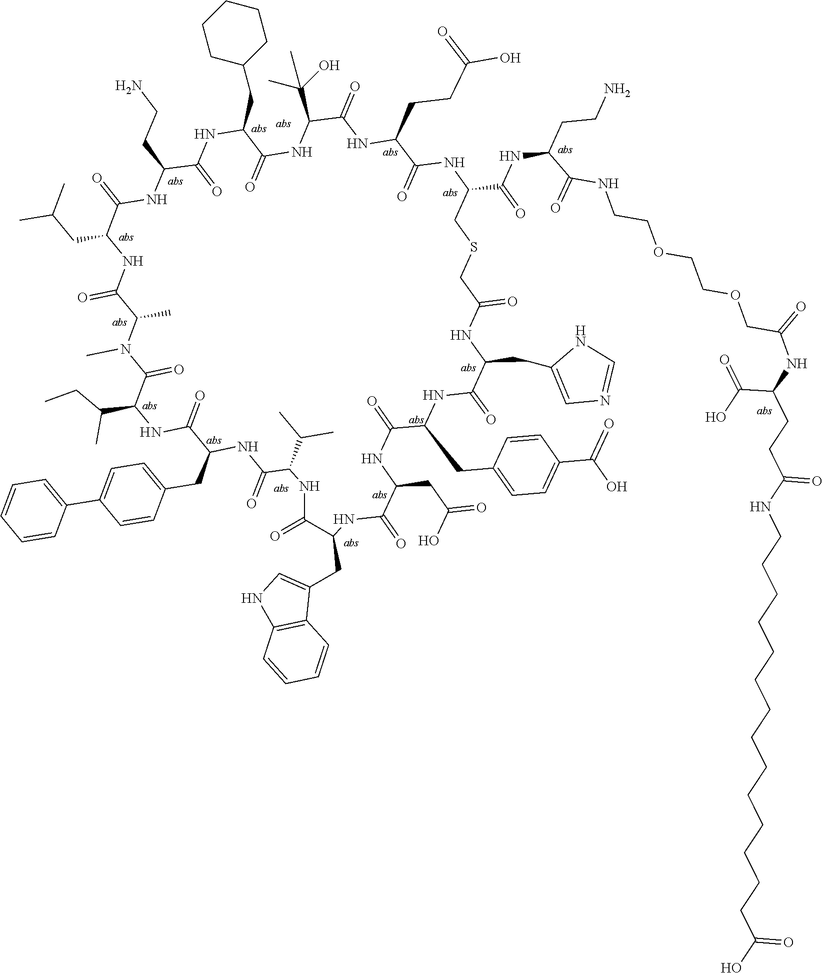 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)N[C@@H](CCC(=O)NCCCCCCCCCCCCCCC(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2633 was prepared on a 50 μmol scale. The yield of the product was 5.6 mg, and its estimated purity by LCMS analysis was 97.1%. Analysis condition B: Retention time=1.95 min; ESI-MS(+) m/z [M+2H]²⁺: 1268.3.

### Preparation of Example 2634

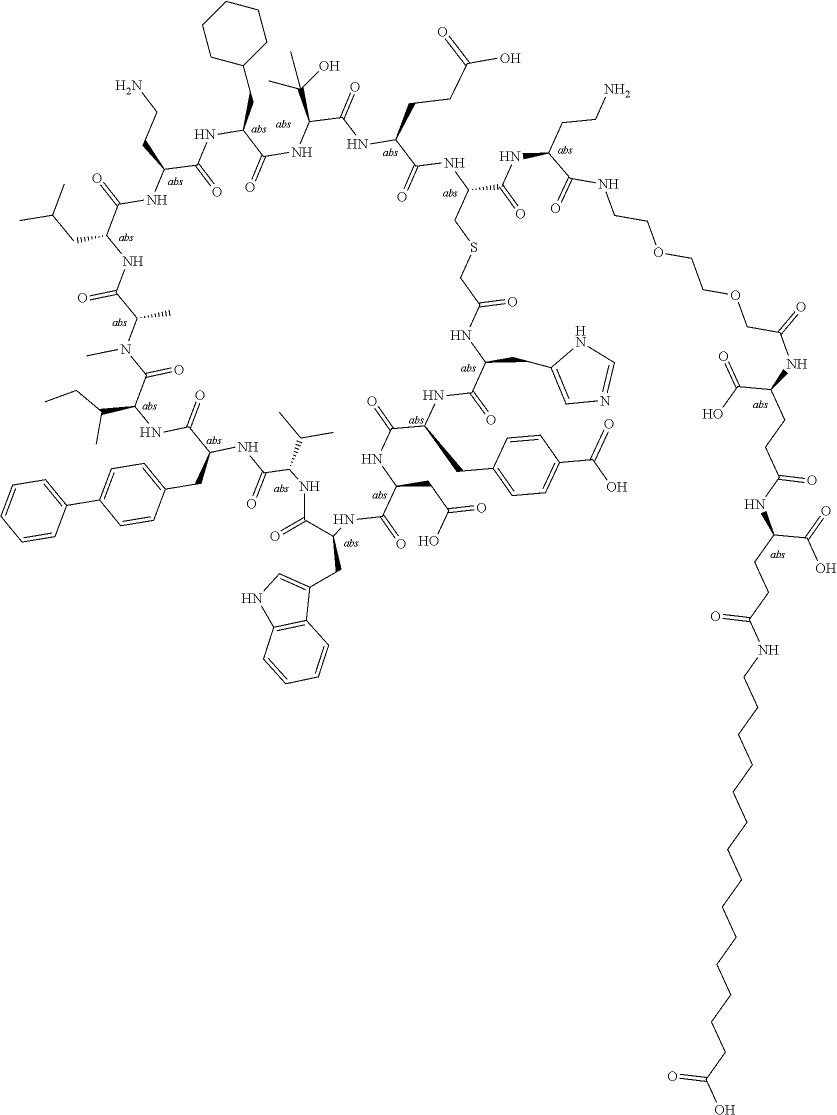 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)N[C@@H](CCC(=O)N[C@H](CCC(=O)NCCCCCCCCCCCCCCC(=O)O)C(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2634 was prepared on a 50 μmol scale. The yield of the product was 10 mg, and its estimated purity by LCMS analysis was 100%. Analysis condition A: Retention time=1.73 min; ESI-MS(+) m/z [M+2H]²⁺: 1333.1.

### Preparation of Example 2635

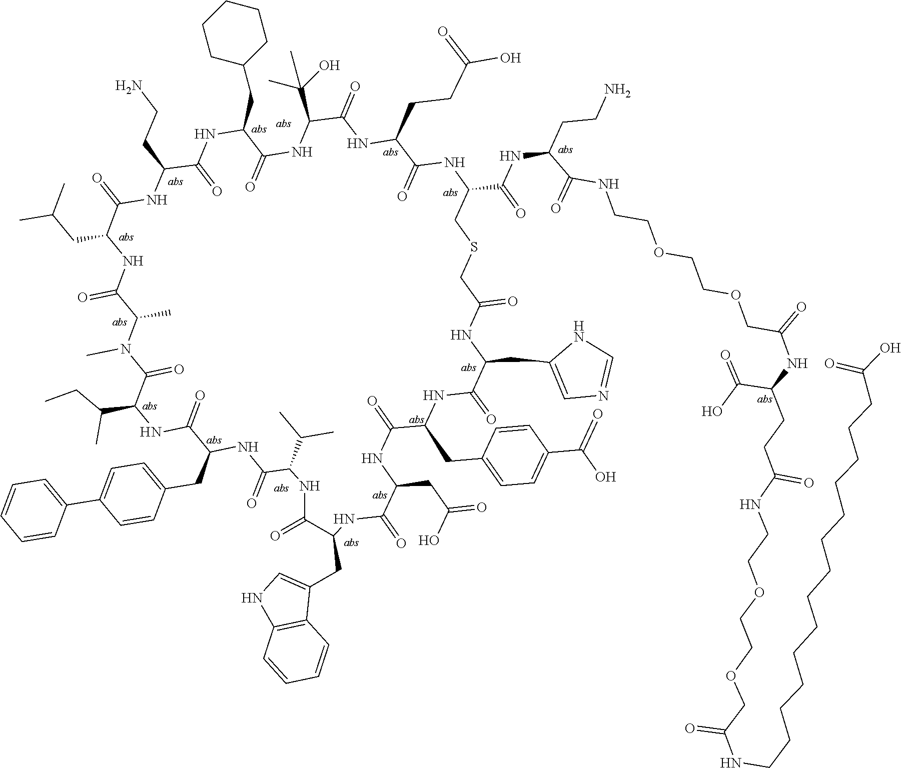 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cnc[nH]2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCCCC(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C1=O]

Example 2635 was prepared on a 50 μmol scale. The yield of the product was 10.5 mg, and its estimated purity by LCMS analysis was 96.1%. Analysis condition B: Retention time=1.95 min; ESI-MS(+) m/z [M+2H]²⁺: 1341.

### Preparation of Example 2636

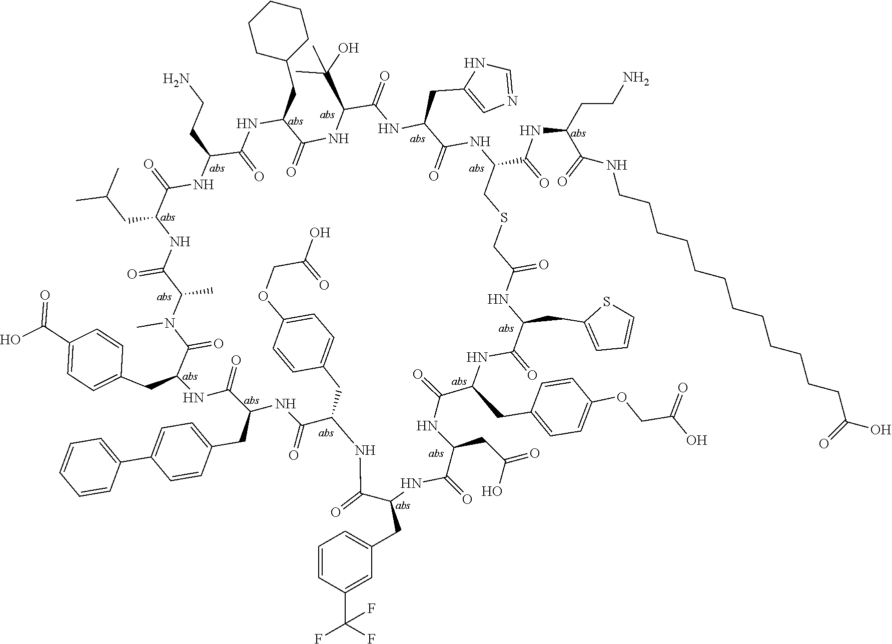 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cccs2)NC(=O)CSC[C@@H](C(=O)N[C@@H](CCN)C(=O)NCCCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC1=O]

Example 2636 was prepared on a 50 μmol scale. The yield of the product was 27.6 mg, and its estimated purity by LCMS analysis was 92.4%. Analysis condition A: Retention time=1.81 min; ESI-MS(+) m/z [M+2H]²⁺: 1259.1.

### Preparation of Example 2637

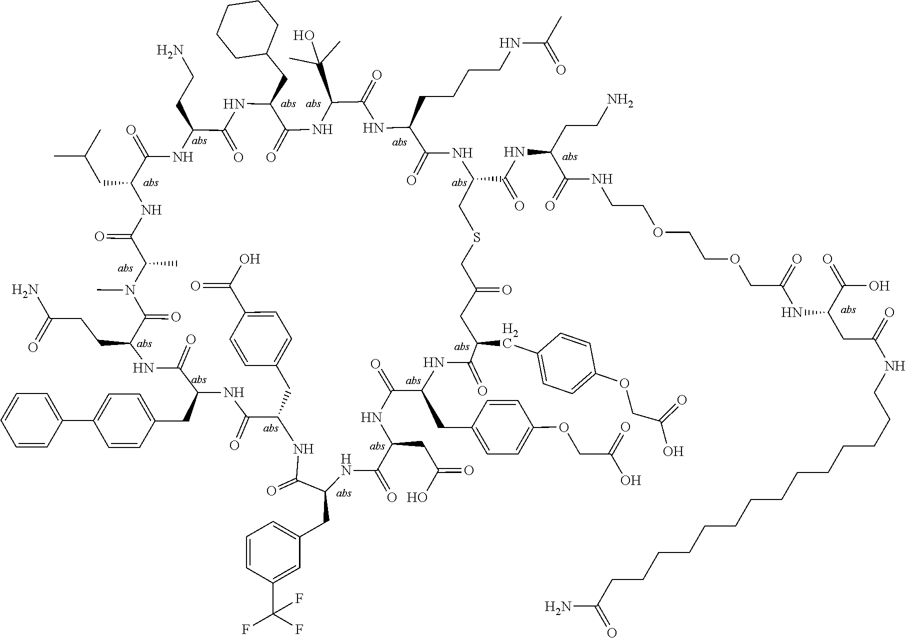 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)CC(=O)CSC[C@@H](C(=O)N[C@@H](CCN)C(=O)NCCOCCOCC(=O)N[C@@H](CC(=O)NCCCCCCCCCCCCCCC(N)=O)C(=O)O)NC1=O]

Example 2637 was prepared on a 50 μmol scale. The yield of the product was 37 mg, and its estimated purity by LCMS analysis was 97.9%. Analysis condition A: Retention time=1.6 min; ESI-MS(+) m/z [M+2H]²⁺: 1407.

### Preparation of Example 2638

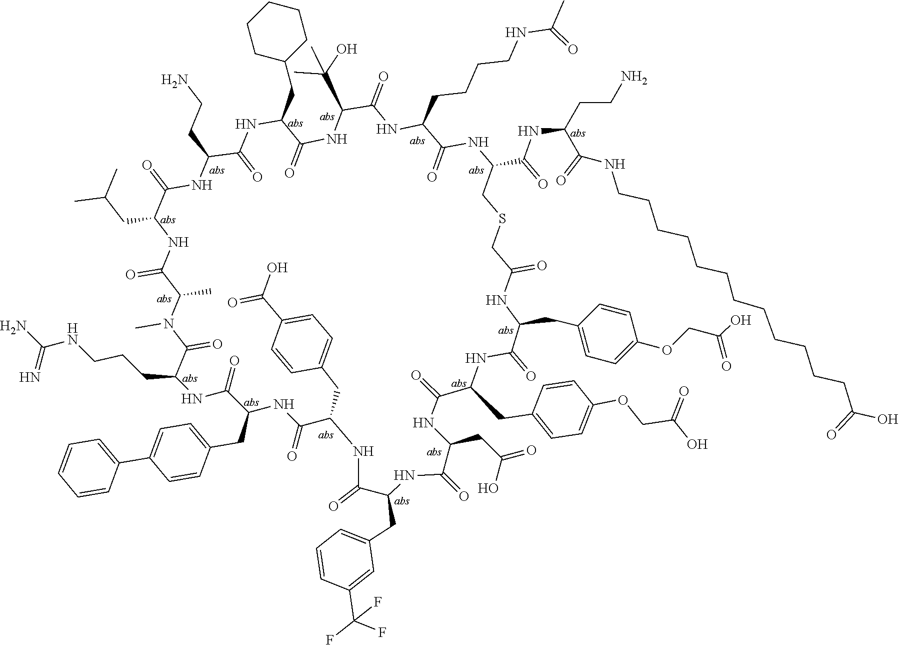 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(=N)N)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Example 2638 was prepared on a 50 μmol scale. The yield of the product was 53.5 mg, and its estimated purity by LCMS analysis was 99.2%. Analysis condition B: Retention time=1.64 min; ESI-MS(+) m/z [M+2H]²⁺: 1277.2.

### Preparation of Example 2639

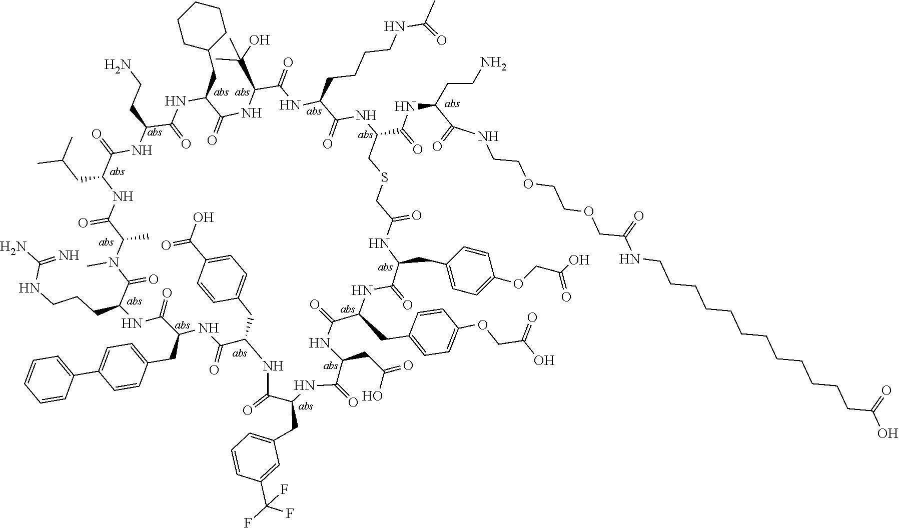 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCCNC(=N)N)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCC(=O)O)NC1=O]

Example 2639 was prepared on a 50 μmol scale. The yield of the product was 11.6 mg, and its estimated purity by L CMS analysis was 100%. Analysis condition A: Retention time=1.67 min; ESI-MS(+) m/z [M+2H]²⁺: 1349.3.

### Preparation of Example 2640

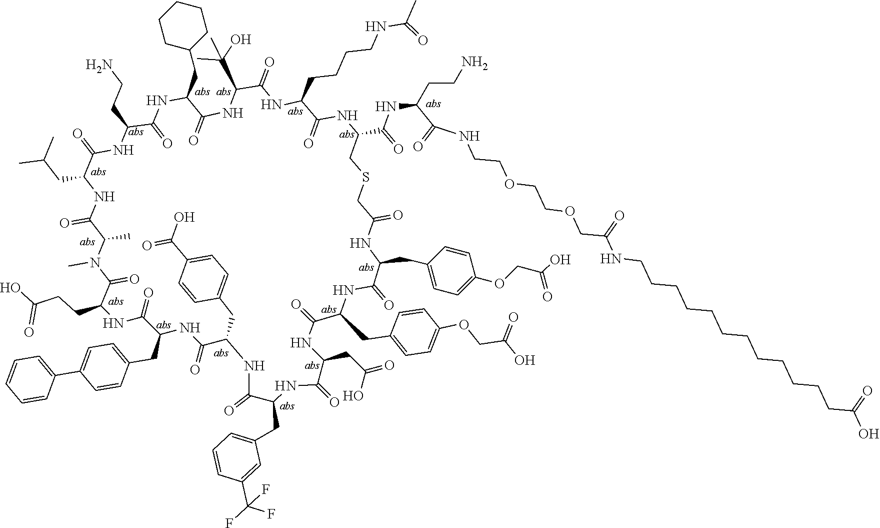 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(=O)O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCC(=O)O)NC1=O]

Example 2640 was prepared on a 50 μmol scale. The yield of the product was 29.9 mg, and its estimated purity by LCMS analysis was 97.9%. Analysis condition B: Retention time=2 min; ESI-MS(+) m/z [M+2H]²⁺: 1336.1.

### Preparation of Example 2641

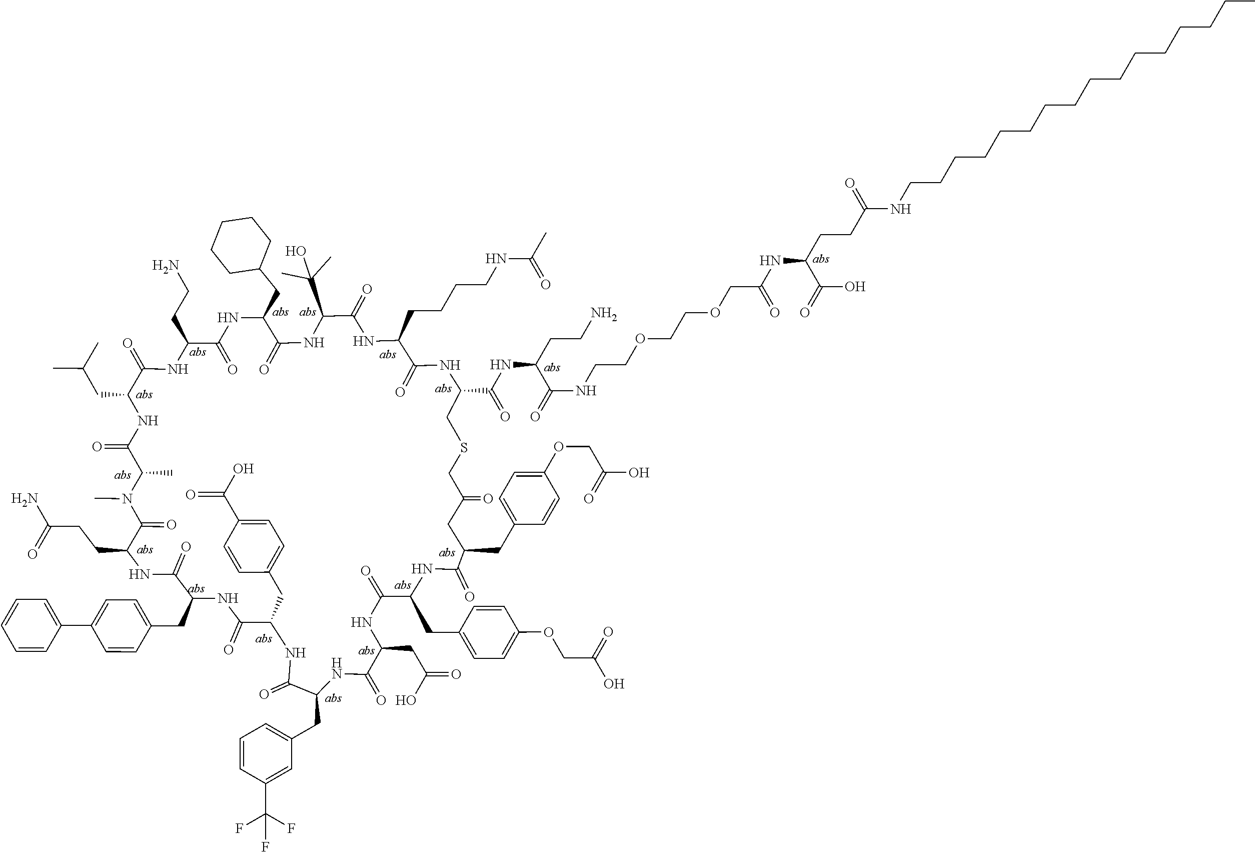 [SMILES: CCCCCCCCCCCCCCCCNC(=O)CC[C@H](NC(=O)COCCOCCNC(=O)[C@H](CCN)NC(=O)[C@@H]1CSCC(=O)C[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C(F)(F)F)c2)C(=O)N[C@@H](Cc2ccc(C(=O)O)cc2)C(=O)N[C@@H](Cc2ccc(-c3ccccc3)cc2)C(=O)N[C@@H](CCC(N)=O)C(=O)N(C)[C@@H](C)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCCNC(C)=O)C(=O)N1)C(=O)O]

Example 2641 was prepared on a 50 μmol scale. The yield of the product was 37.1 mg, and its estimated purity by LCMS analysis was 96.9%. Analysis condition A: Retention time=2.17 min; ESI-MS(+) m/z [M+2H]²⁺: 1406.

### Preparation of Example 2642

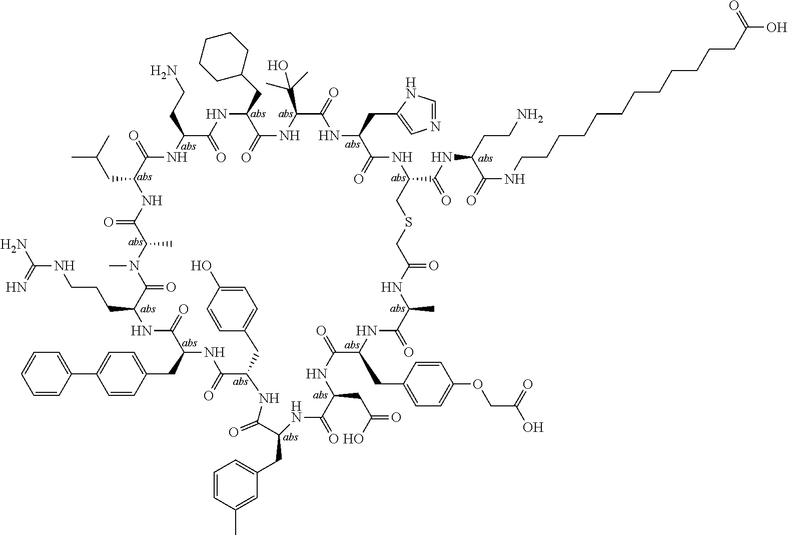 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](Cc3ccc(O)cc3)NC2=O)c1]

Example 2642 was prepared on a 50 μmol scale. The yield of the product was 17.4 mg, and its estimated purity by LCMS analysis was 87.1%. Analysis condition A: Retention time=1.63 min; ESI-MS(+) m/z [M+3H]²⁺: 763.

### Preparation of Example 2643

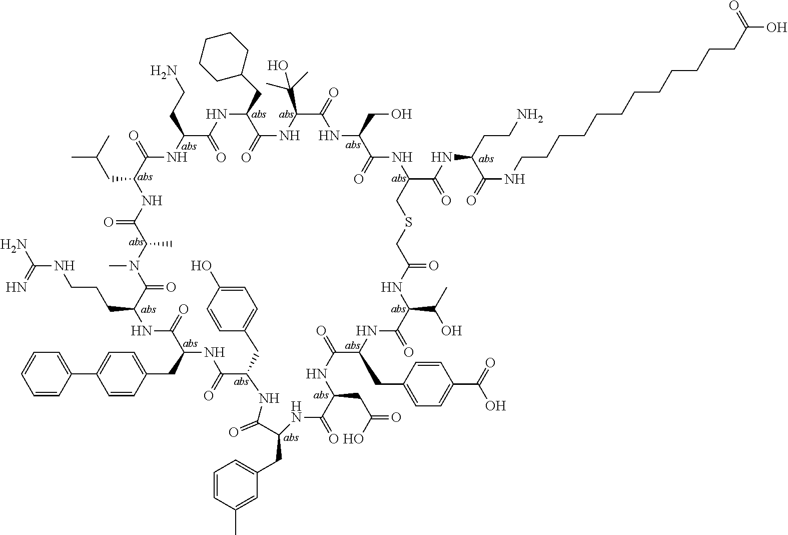 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSCC(C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](Cc3ccc(O)cc3)NC2=O)c1]

Example 2643 was prepared on a 50 μmol scale. The yield of the product was 11.3 mg, and its estimated purity by LCMS analysis was 97.8%. Analysis condition A: Retention time=1.77 min; ESI-MS(+) m/z [M+2H]²⁺: 1119.1.

### Preparation of Example 2644

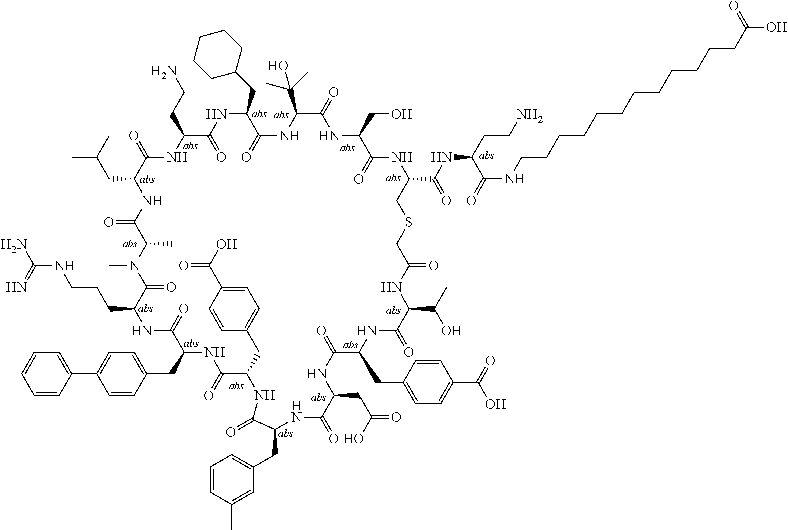 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=N)N)NC(=O)[C@H](Cc3ccc(-c4ccccc4)cc3)NC(=O)[C@H](Cc3ccc(C(=O)O)cc3)NC2=O)c1]

Example 2644 was prepared on a 50 μmol scale. The yield of the product was 15.7 mg, and its estimated purity by LCMS analysis was 96.6%. Analysis condition B: Retention time=1.52 min; ESI-MS(+) m/z [M+3H]³⁺: 756.2.

### Preparation of Example 2645

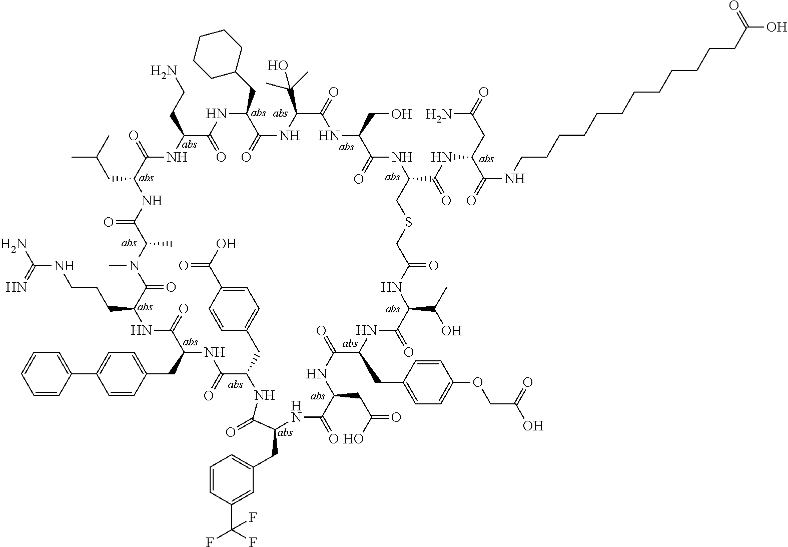 [SMILES: CC(C)C[C@H]1NC(=O)[C@H](C)N(C)C(=O)[C@H](CCCNC(=N)N)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Example 2645 was prepared on a 50 μmol scale. The yield of the product was 20.9 mg, and its estimated purity by LCMS analysis was 95.2%. Analysis condition B: Retention time=1.58 min; ESI-MS(+) m/z [M+2H]²⁺: 1181.9.

### Preparation of Example 2646

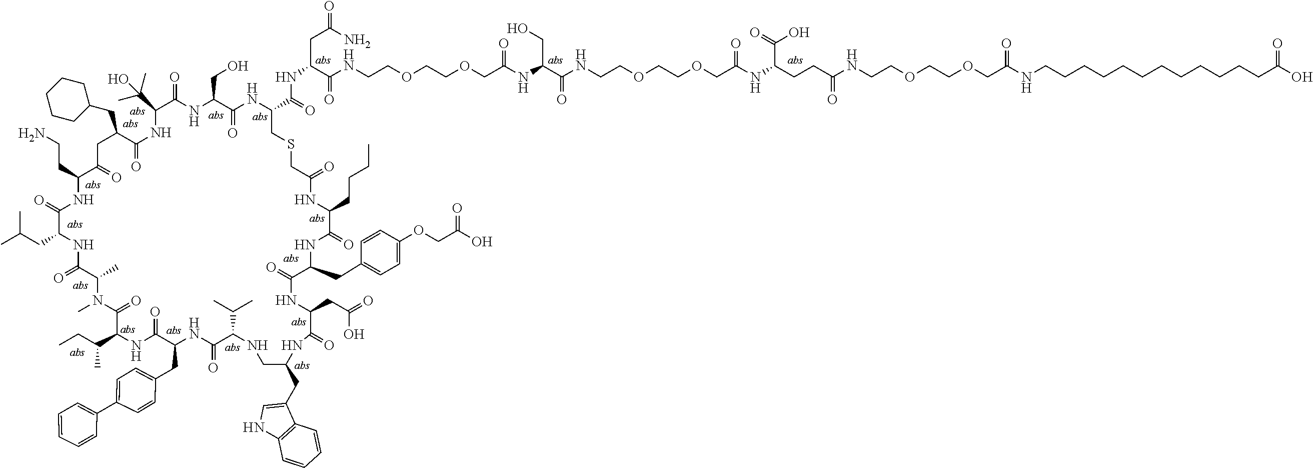 [SMILES: CCCC[C@@H]1NC(=O)CSC[C@@H](C(=O)N[C@H](CC(N)=O)C(=O)NCCOCCOCC(=O)N[C@@H](CO)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)CC(=O)[C@H](CCN)NC(=O)[C@@H](CC(C)C)NC(=O)[C@H](C)N(C)C(=O)[C@H]([C@H](C)CC)NC(=O)[C@H](Cc2ccc(-c3ccccc3)cc2)NC(=O)[C@H](C(C)C)NC[C@H](Cc2c[nH]c3ccccc23)NC(=O)[C@H](CC(=O)O)NC(=O)[C@H](Cc2ccc(OCC(=O)O)cc2)NC1=O]

Example 2646 was prepared on a 50 μmol scale. The yield of the product was 6.4 mg, and its estimated purity by LCMS analysis was 96.1%. Analysis condition A: Retention time=1.7 min; ESI-MS(+) m/z [M+2H]²⁺: 1432.3.

### Preparation of Example 2647

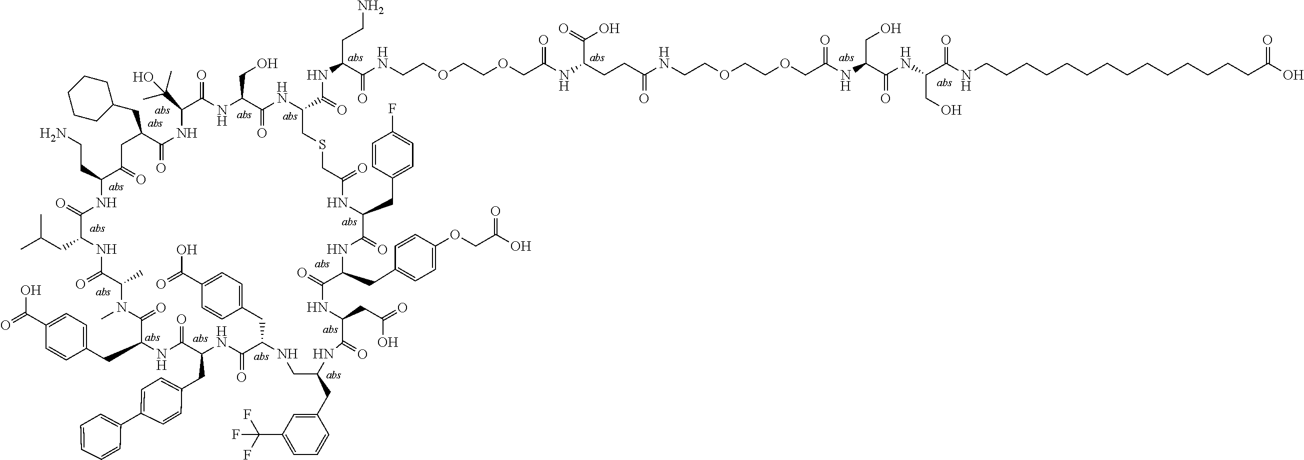 [SMILES: CC(C)C[C@H]1NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(F)cc2)NC(=O)CSC[C@@H](C(=O)N[C@@H](CCN)C(=O)NCCOCCOCC(=O)N[C@@H](CCC(=O)NCCOCCOCC(=O)N[C@@H](CO)C(=O)N[C@@H](CO)C(=O)NCCCCCCCCCCCCCCC(=O)O)C(=O)O)NC(=O)[C@H](CO)NC(=O)[C@H](C(C)(C)O)NC(=O)[C@H](CC2CCCCC2)CC(=O)[C@H](CCN)NC1=O]

Example 2647 was prepared on a 50 μmol scale. The yield of the product was 3.2 mg, and its estimated purity by LCMS analysis was 96.1%. Analysis condition B: Retention time=2.15 min; ESI-MS(+) m/z [M+2H]²⁺: 1536.

### Preparation of Example 2648

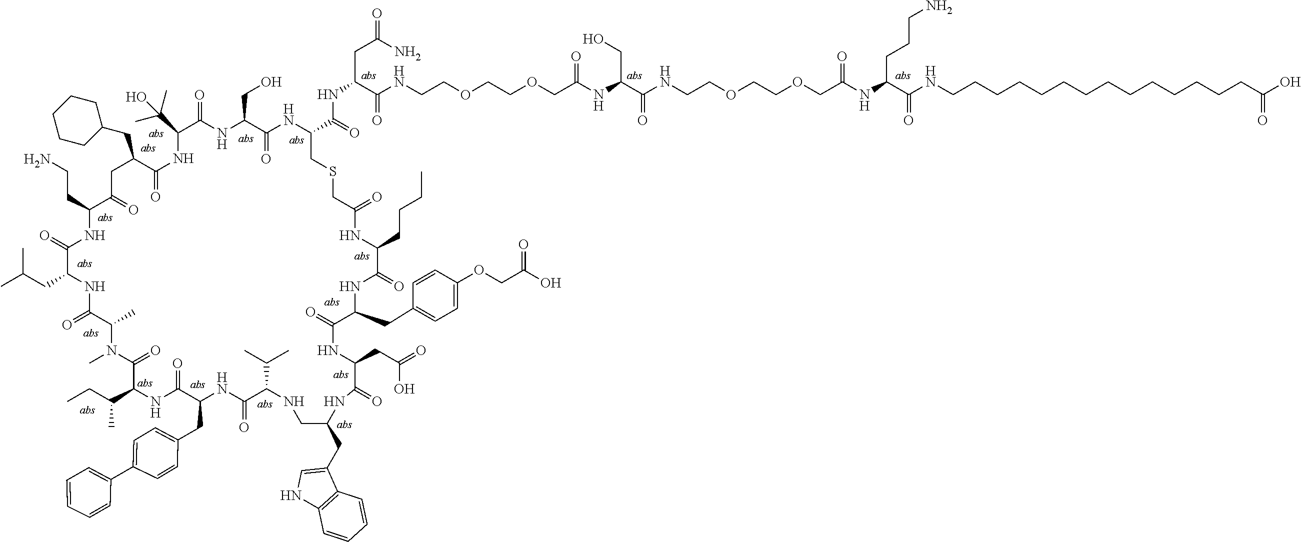 [SMILES: CCCC[C@@H]1NC(=O)CSC[C@@H](C(=O)N[C@H](CC(N)=O)C(=O)NCCOCCOCC(=O)N[C@@H](CO)C(=O)NCCOCCOCC(=O)N[C@@H](CCCN)C(=O)NCCCCCCCCCCCCCCC(=O)O)NC(=O)[C@H](CO)NC(=O)[C@H](C(C)(C)O)NC(=O)[C@H](CC2CCCCC2)CC(=O)[C@H](CCN)NC(=O)[C@@H](CC(C)C)NC(=O)[C@H](C)N(C)C(=O)[C@H]([C@H](C)CC)NC(=O)[C@H](Cc2ccc(-c3ccccc3)cc2)NC(=O)[C@H](C(C)C)NC[C@H](Cc2c[nH]c3ccccc23)NC(=O)[C@H](CC(=O)O)NC(=O)[C@H](Cc2ccc(OCC(=O)O)cc2)NC1=O]

Example 2648 was prepared on a 50 μmol scale. The yield of the product was 3.9 mg, and its estimated purity by LCMS analysis was 95.6%. Analysis condition A: Retention time=1.92 min; ESI-MS(+) m/z [M+2H]²⁺: 1366.1.

### Preparation of Example 2649

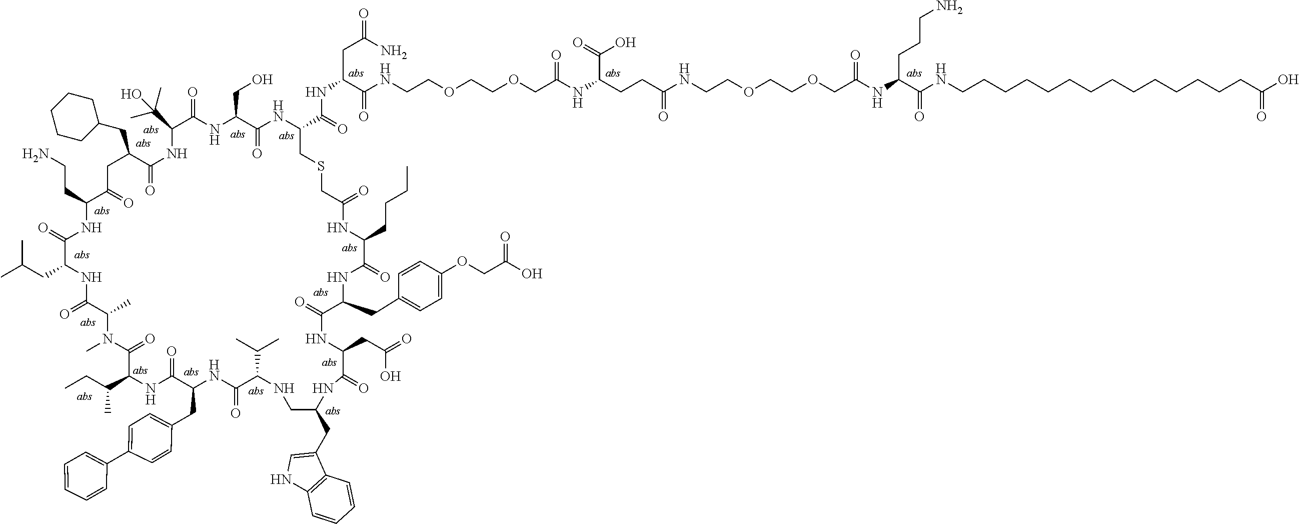 [SMILES: CCCC[C@@H]1NC(=O)CSC[C@@H](C(=O)N[C@H](CC(N)=O)C(=O)NCCOCCOCC(=O)N[C@@H](CCC(=O)NCCOCCOCC(=O)N[C@@H](CCCN)C(=O)NCCCCCCCCCCCCCCC(=O)O)C(=O)O)NC(=O)[C@H](CO)NC(=O)[C@H](C(C)(C)O)NC(=O)[C@H](CC2CCCCC2)CC(=O)[C@H](CCN)NC(=O)[C@@H](CC(C)C)NC(=O)[C@H](C)N(C)C(=O)[C@H]([C@H](C)CC)NC(=O)[C@H](Cc2ccc(-c3ccccc3)cc2)NC(=O)[C@H](C(C)C)NC[C@H](Cc2c[nH]c3ccccc23)NC(=O)[C@H](CC(=O)O)NC(=O)[C@H](Cc2ccc(OCC(=O)O)cc2)NC1=O]

Example 2649 was prepared on a 50 μmol scale. The yield of the product was 15.9 mg, and its estimated purity by LCMS analysis was 91.2%. Analysis condition A: Retention time=1.9 min; ESI-MS(+) m/z [M+2H]²⁺: 1387.

### Preparation of Example 2650

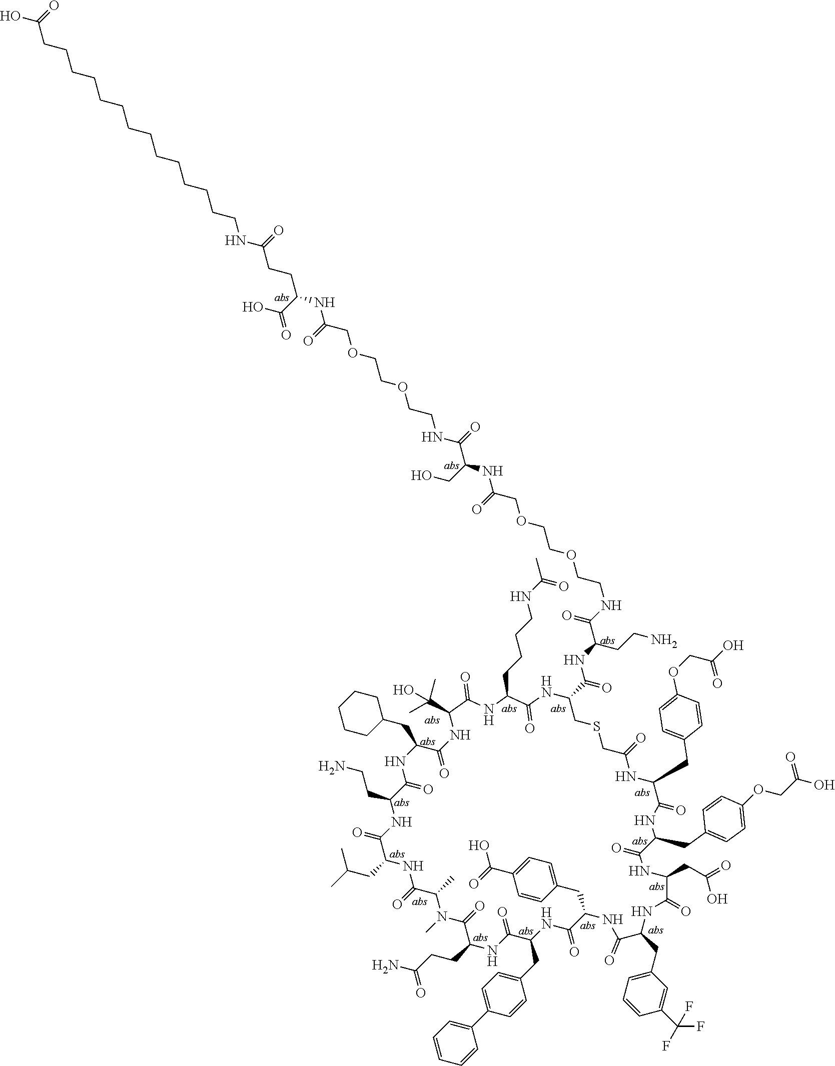 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CCN)C(=O)NCCOCCOCC(=O)N[C@@H](CO)C(=O)NCCOCCOCC(=O)N[C@@H](CCC(=O)NCCCCCCCCCCCCCCC(=O)O)C(=O)O)NC1=O]

Example 2650 was prepared on a 50 μmol scale. The yield of the product was 16.3 mg, and its estimated purity by LCMS analysis was 97.7%. Analysis condition B: Retention time=2.09 min; ESI-MS(+) m/z [M+2H]²⁺: 1531.

### Preparation of Example 2651

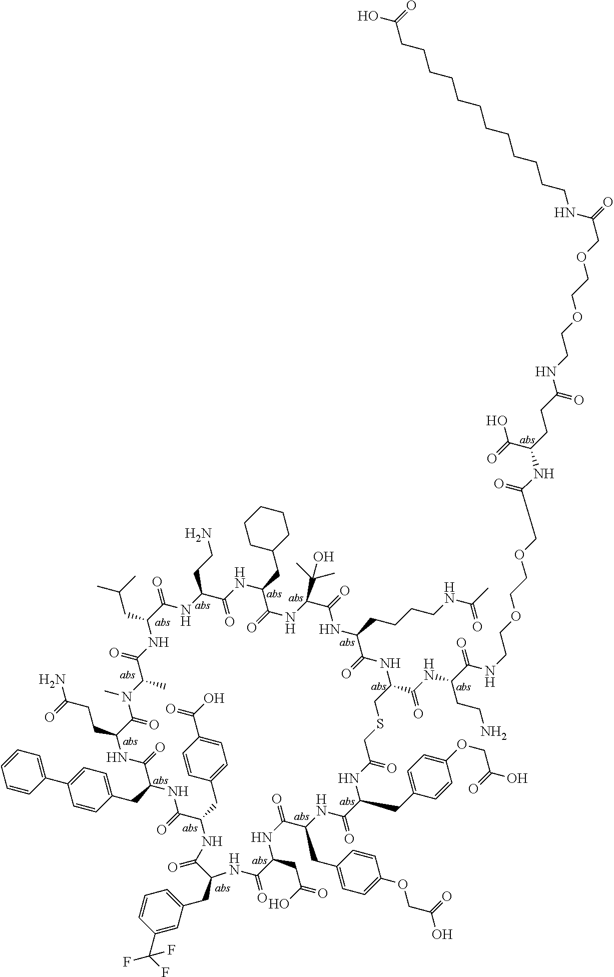 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC1=O]

Example 2651 was prepared on a 50 μmol scale. The yield of the product was 4.7 mg, and its estimated purity by LCMS analysis was 97.1%. Analysis condition B: Retention time=1.99 min; ESI-MS(+) m/z [M+2H]²⁺: 982.1.

### Preparation of Example 2652

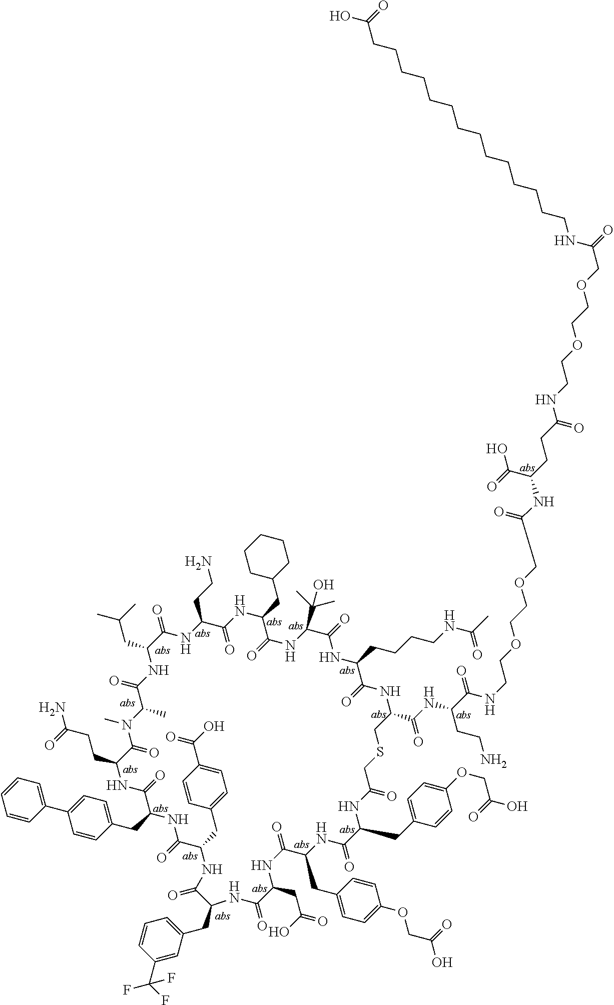 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCCCC(=O)O)C(=O)O)NC1=O]

Example 2652 was prepared on a 50 μmol scale. The yield of the product was 7.8 mg, and its estimated purity by LCMS analysis was 96.2%. Analysis condition B: Retention time=2 min; ESI-MS(+) m/z [M+3H]³: 992.2.

### Preparation of Example 2653

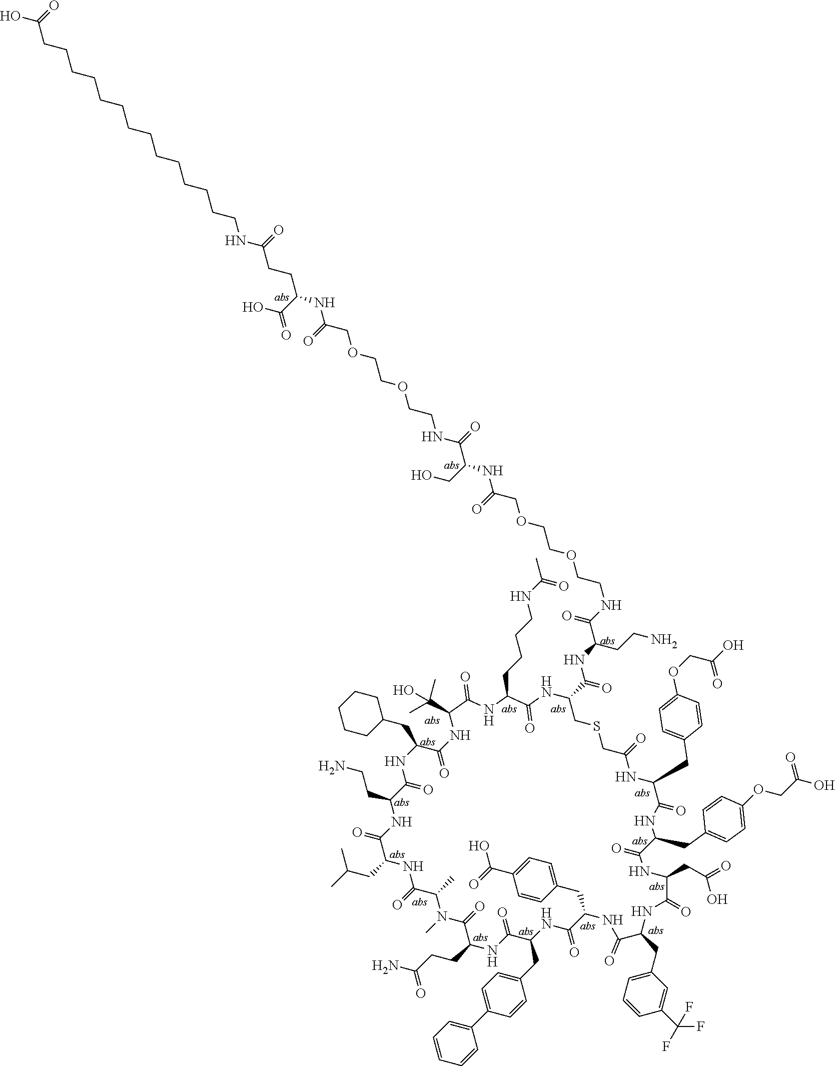 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CCN)C(=O)NCCOCCOCC(=O)N[C@H](CO)C(=O)NCCOCCOCC(=O)N[C@@H](CCC(=O)NCCCCCCCCCCCCCCC(=O)O)C(=O)O)NC1=O]

Example 2653 was prepared on a 50 μmol scale. The yield of the product was 15.5 mg, and its estimated purity by LCMS analysis was 99%. Analysis condition B: Retention time=2.07 min; ESI-MS(+) m/z [M+2H]²⁺: 1551.1.

### Preparation of Example 2654

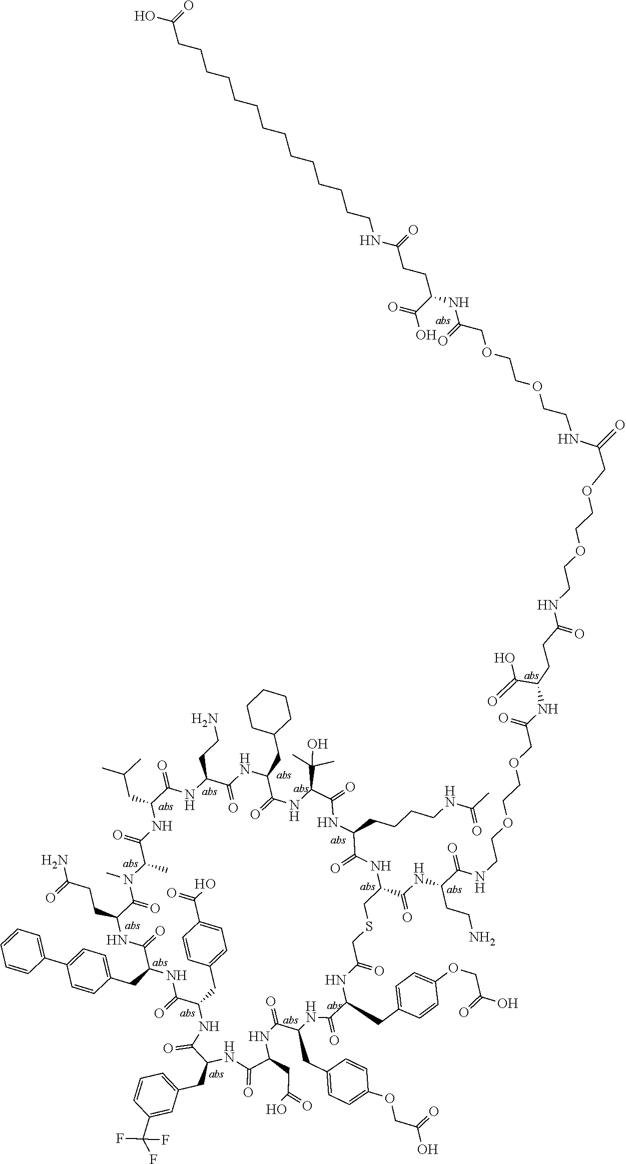 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@@H](CCC(=O)NCCOCCOCC(=O)NCCOCCOCC(=O)N[C@@H](CCC(=O)NCCCCCCCCCCCCCCC(=O)O)C(=O)O)C(=O)O)NC1=O]

Example 2654 was prepared on a 50 μmol scale. The yield of the product was 15.8 mg, and its estimated purity by LCMS analysis was 95.1%. Analysis condition B: Retention time=2.06 min; ESI-MS(+) m/z [M+2H]²⁺: 1083.2.

### Preparation of Example 2655

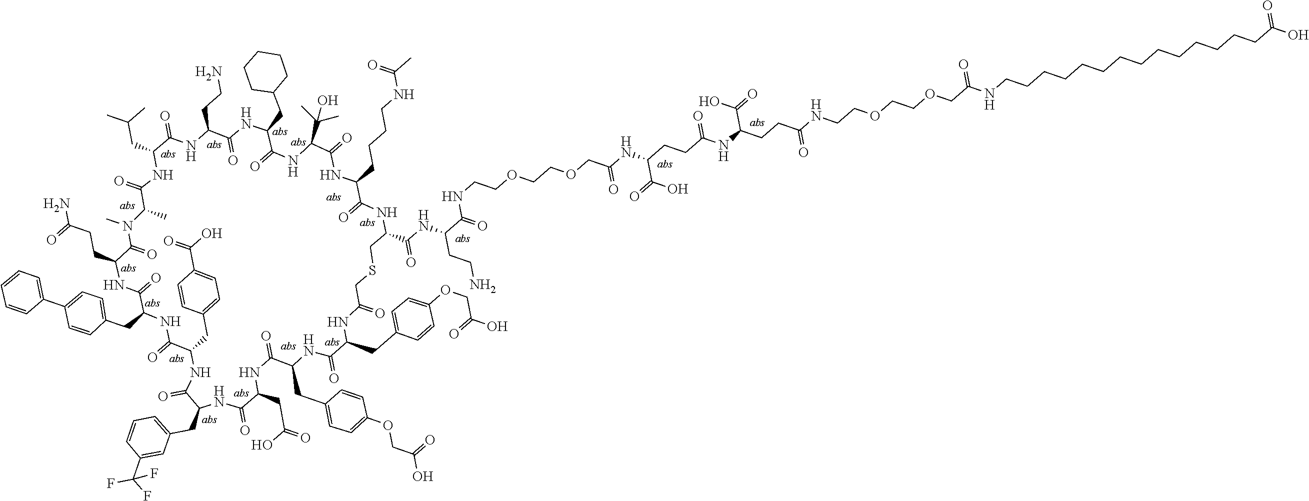 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@H](CCC(=O)N[C@H](CCC(=O)NCCOCCOCC(=O)NCCCCCCCCCCCCCCC(=O)O)C(=O)O)C(=O)O)NC1=O]

Example 2655 was prepared on a 50 μmol scale. The yield of the product was 5.7 mg, and its estimated purity by LCMS analysis was 97.9%. Analysis condition A: Retention time=1.61 min; ESI-MS(+) m/z [M+2H]²⁺: 1551.2.

### Preparation of Example 2656

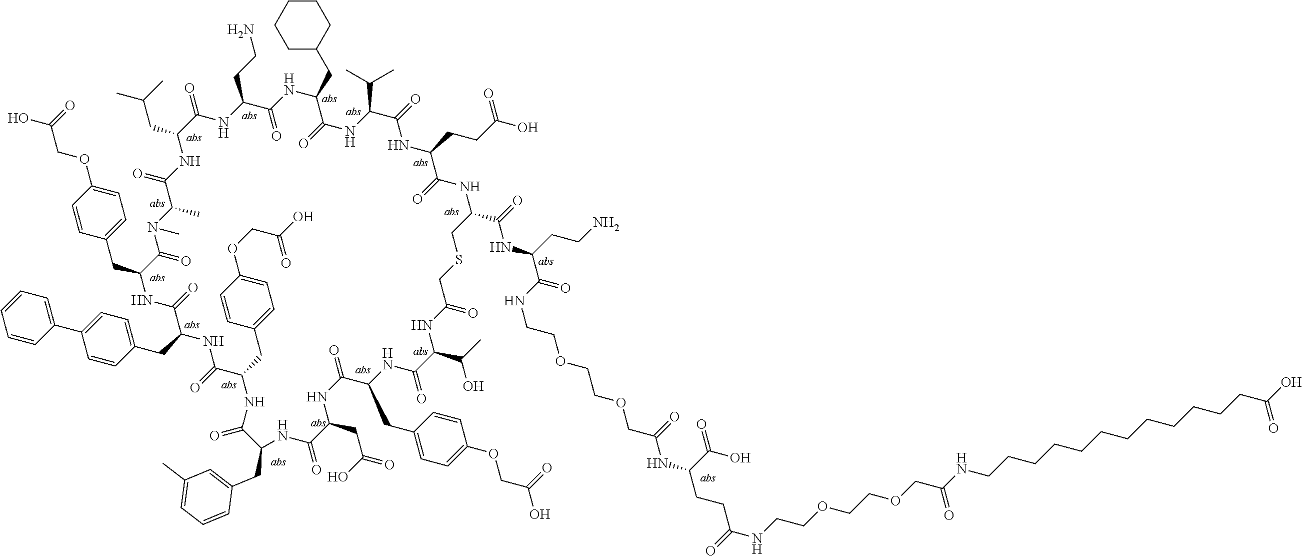 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2656 was prepared on a 50 μmol scale. The yield of the product was 15.8 mg, and its estimated purity by LCMS analysis was 96.8%. Analysis condition A: Retention time=1.62 min; ESI-MS(+) m/z [M+2H]²⁺: 1418.2.

### Preparation of Example 2657

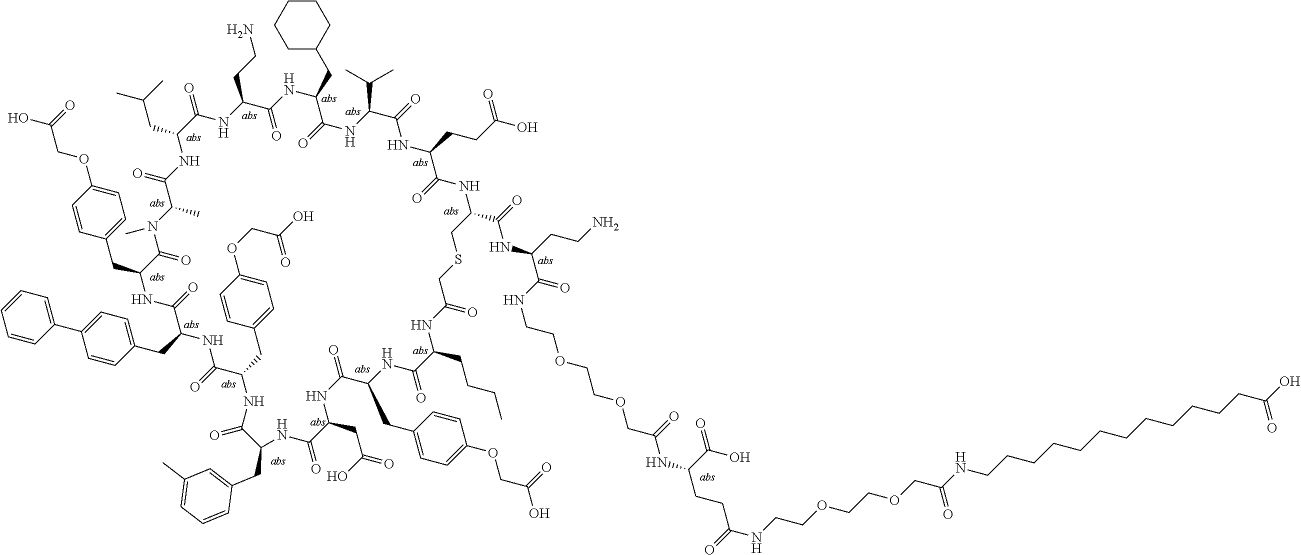 [SMILES: CCCC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Example 2657 was prepared on a 50 μmol scale. The yield of the product was 20.1 mg, and its estimated purity by LCMS analysis was 96.5%. Analysis condition 1: Retention time=2.08 min; ESI-MS(+) m/z [M+2H]²⁺: 1423.9.

### Preparation of Example 2658

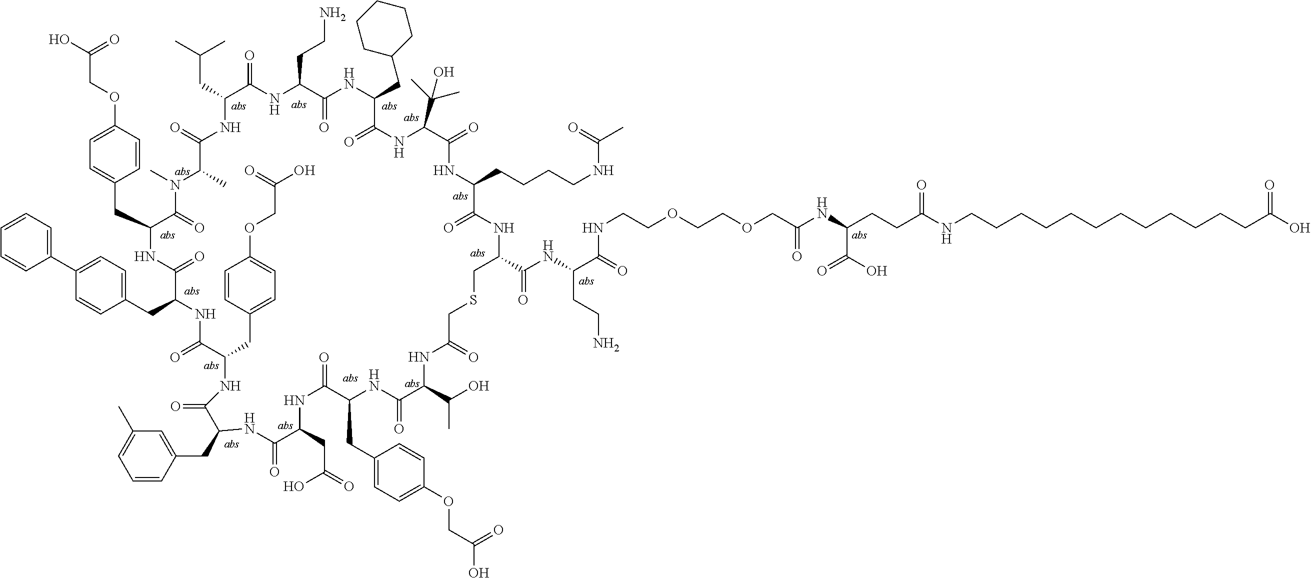 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCCCCCCCCCCCC(=O)O)C(=O)O)NC1=O]

Example 2658 was prepared on a 50 μmol scale. The yield of the product was 15.2 mg, and its estimated purity by LCMS analysis was 100%. Analysis condition 1: Retention time=1.63 min; ESI-MS(+) m/z [M+2H]²⁺: 1374.7.

### Preparation of Example 2659

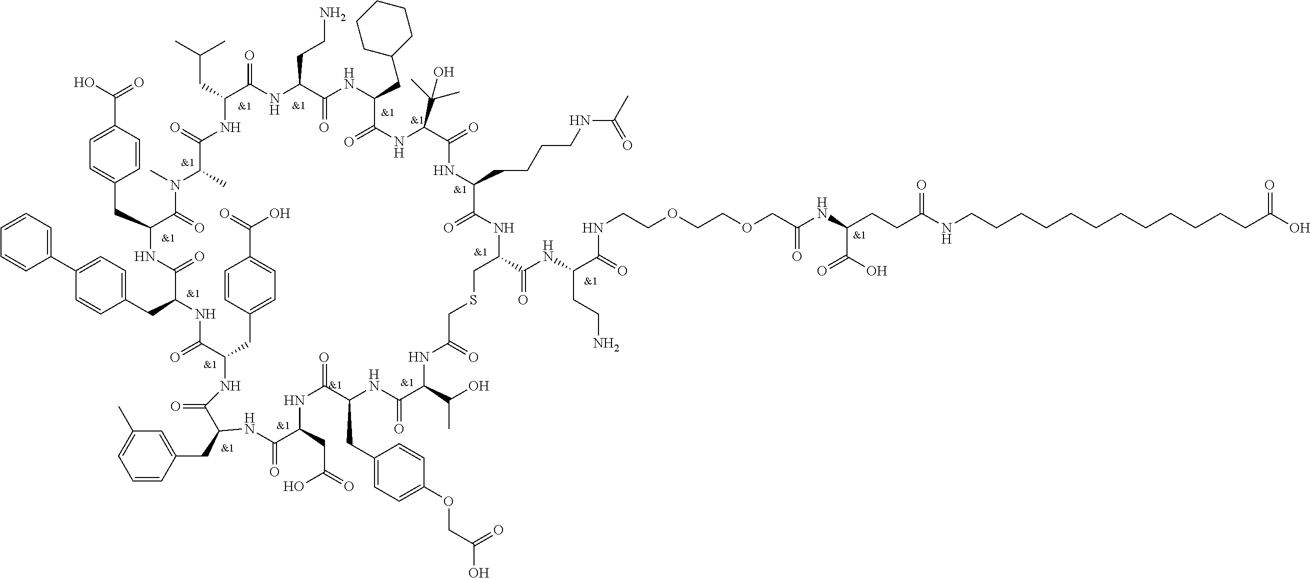 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCCCCCCCCCCCC(=O)O)C(=O)O)NC1=O]

Example 2659 was prepared on a 50 μmol scale. The yield of the product was 8.2 mg, and its estimated purity by LCMS analysis was 100%. Analysis condition 1: Retention time=1.49 min; ESI-MS(+) m/z [M+2H]²⁺: 1345.

### Preparation of Example 2660

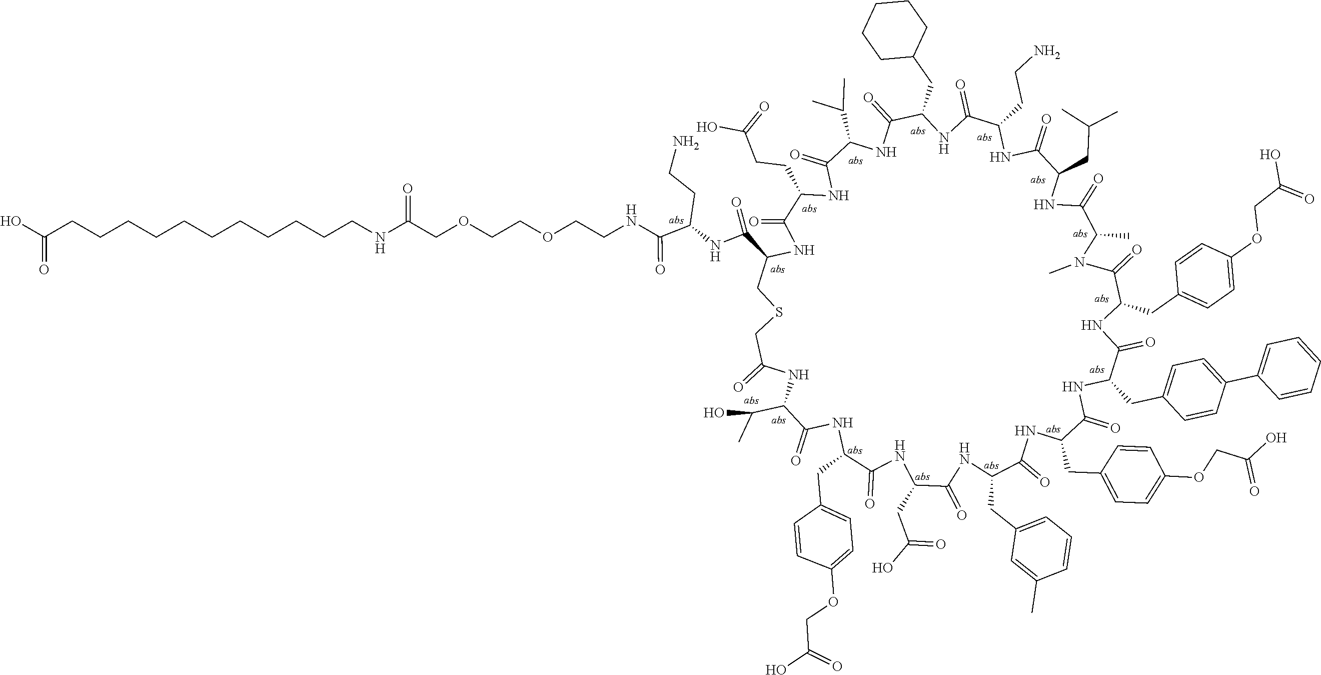 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2660 was prepared on a 100 μmol scale. The yield of the product was 57 mg, and its estimated purity by LCMS analysis was 86.5%. Analysis condition A: Retention time=1.5 min; ESI-MS(+) m/z [M+3H]³⁺: 849.8.

### Preparation of Example 2661

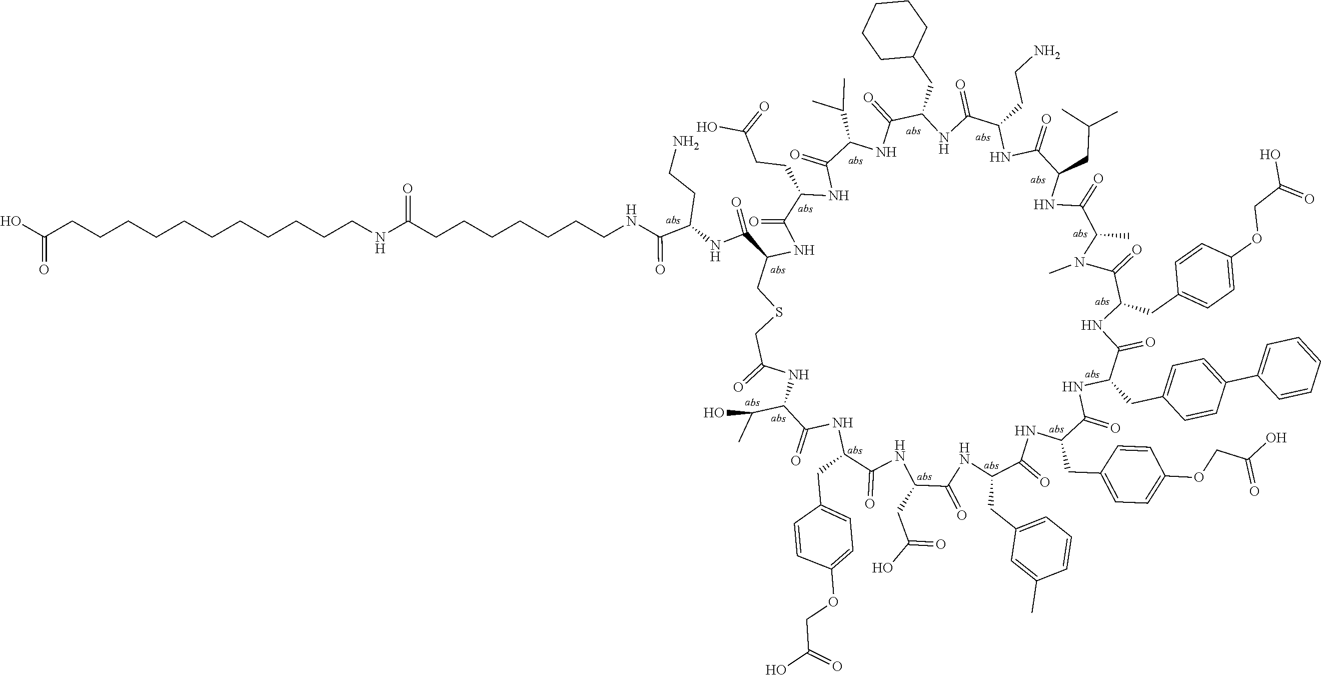 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2661 was prepared on a 100 μmol scale. The yield of the product was 48.7 mg, and its estimated purity by LCMS analysis was 85%. Analysis condition A: Retention time=1.56 min; ESI-MS(+) m/z [M+3H]³⁺: 848.2.

### Preparation of Example 2662

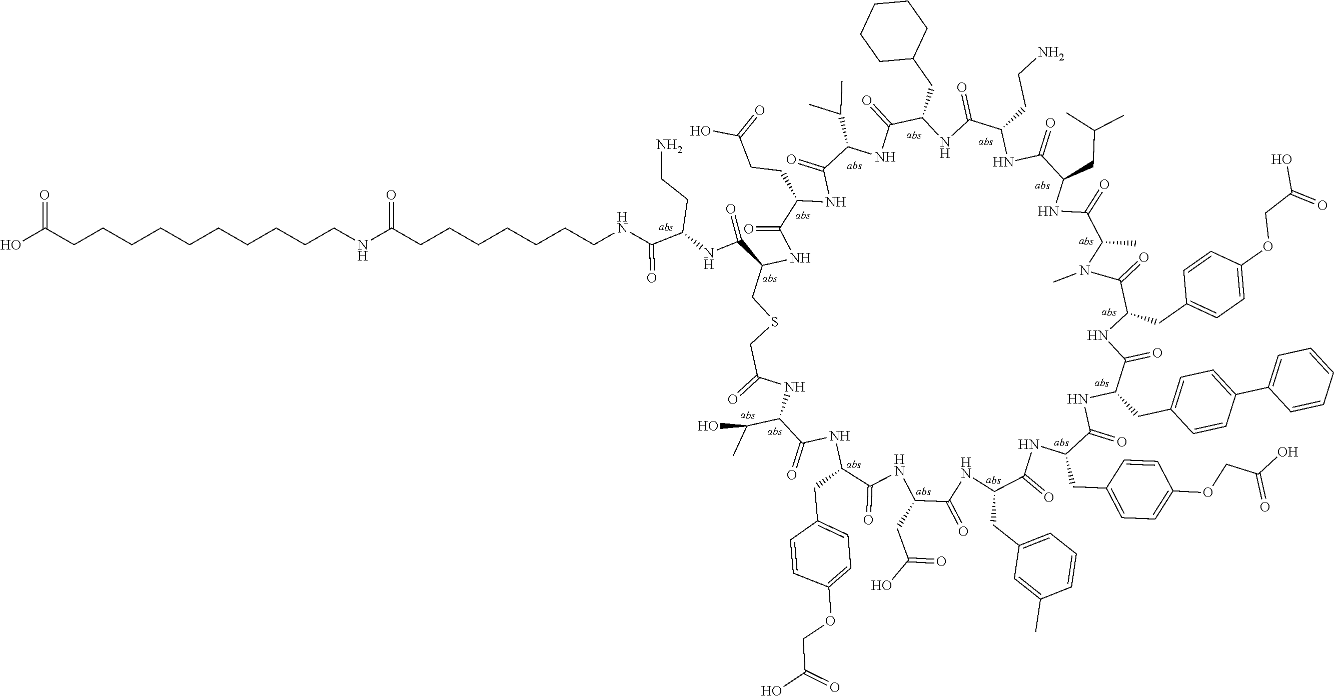 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2662 was prepared on a 100 μmol scale. The yield of the product was 43.5 mg, and its estimated purity by LCMS analysis was 92.9%. Analysis condition A: Retention time=1.54 min; ESI-MS(+) m/z [M+3H]³⁺: 844.1.

### Preparation of Example 2663

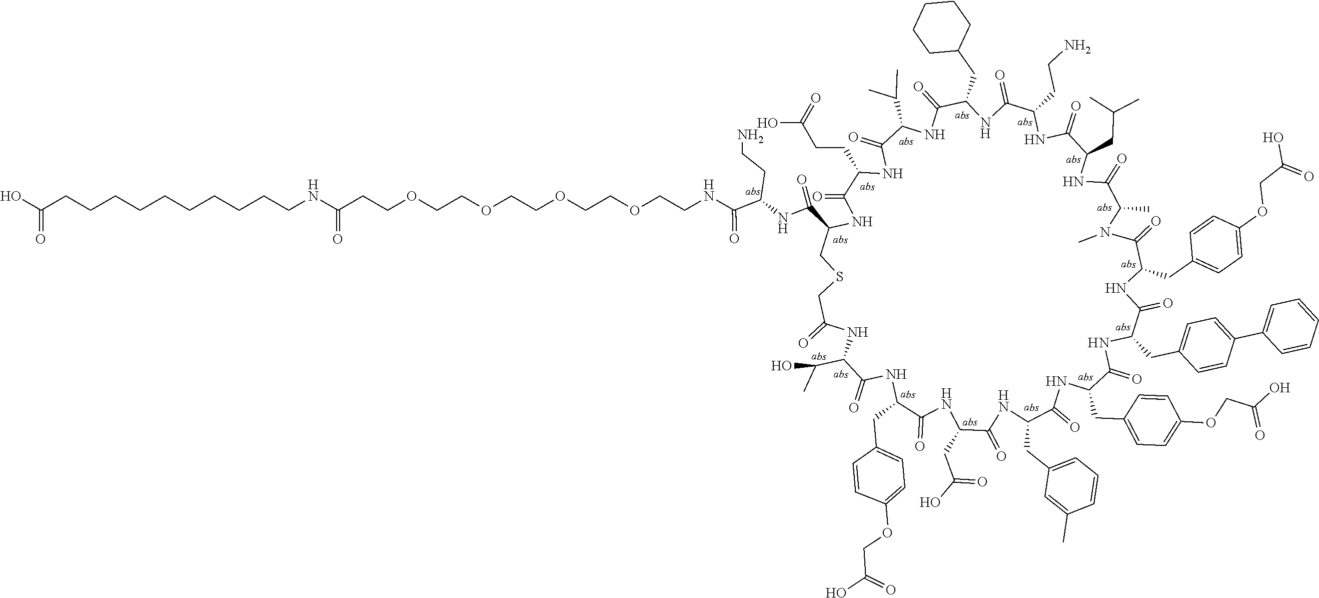 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCCOCCOCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2663 was prepared on a 100 μmol scale. The yield of the product was 72.6 mg, and its estimated purity by LCMS analysis was 91.2%. Analysis condition A: Retention time=1.48 min; ESI-MS(+) m/z [M+3H]³⁺: 879.3.

### Preparation of Example 2664

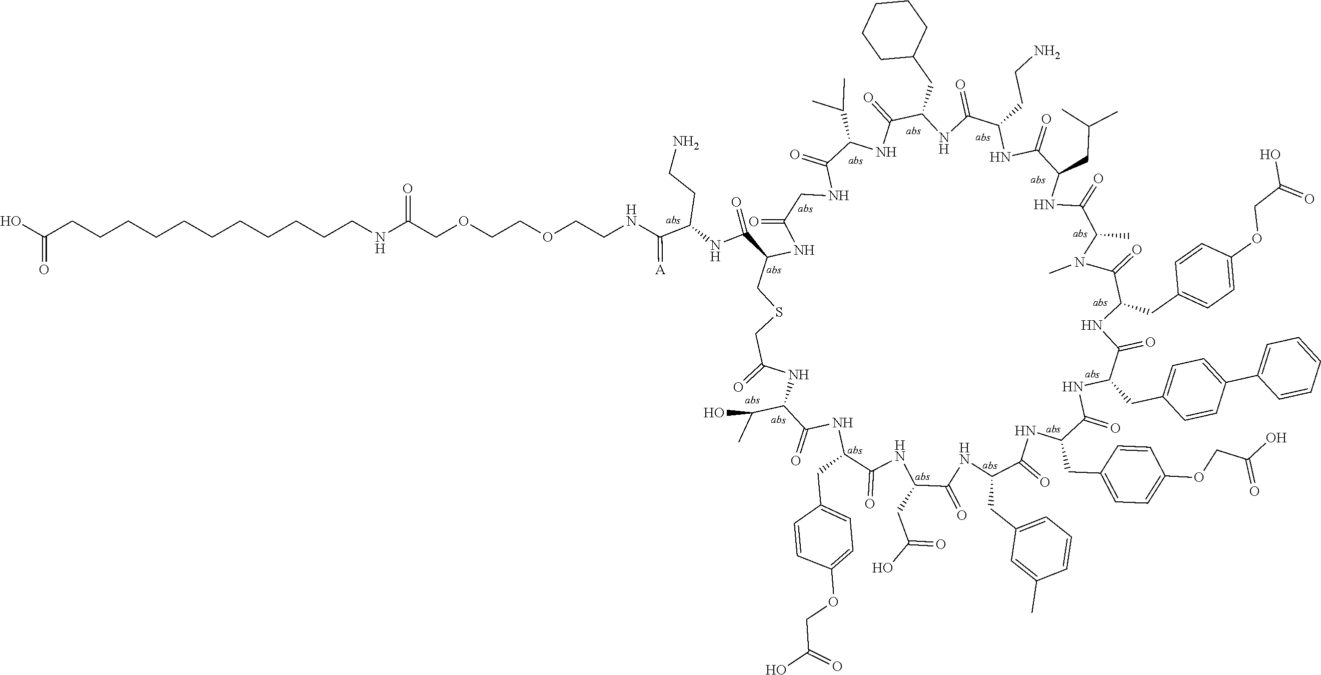

Example 2664 was prepared on a 100 μmol scale. The yield of the product was 12.8 mg, and its estimated purity by LCMS analysis was 99.1%. Analysis condition B: Retention time=1.96 min; ESI-MS(+) m/z [M+2H]²⁺: 1238.2.

### Preparation of Example 2665

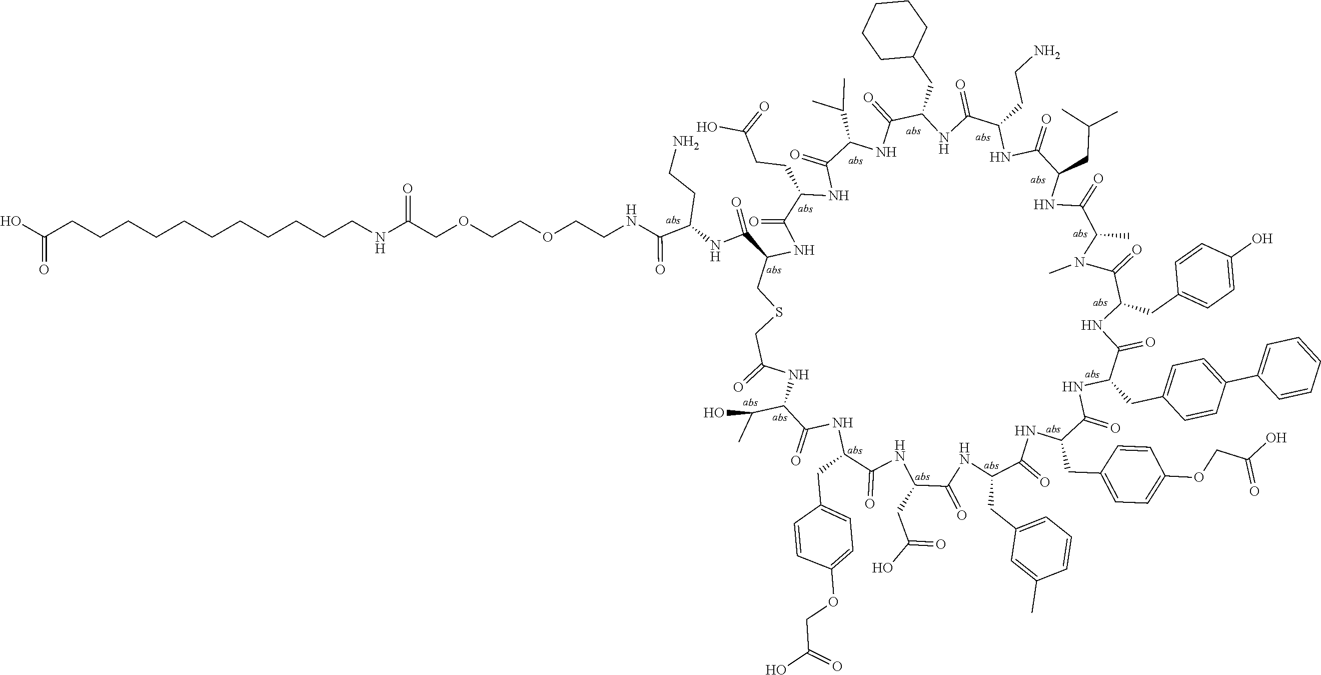 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2665 was prepared on a 100 μmol scale. The yield of the product was 29.2 mg, and its estimated purity by LCMS analysis was 96.7%. Analysis condition B: Retention time=1.87 min; ESI-MS(+) m/z [M+3H]³⁺: 830.6.

### Preparation of Example 2666

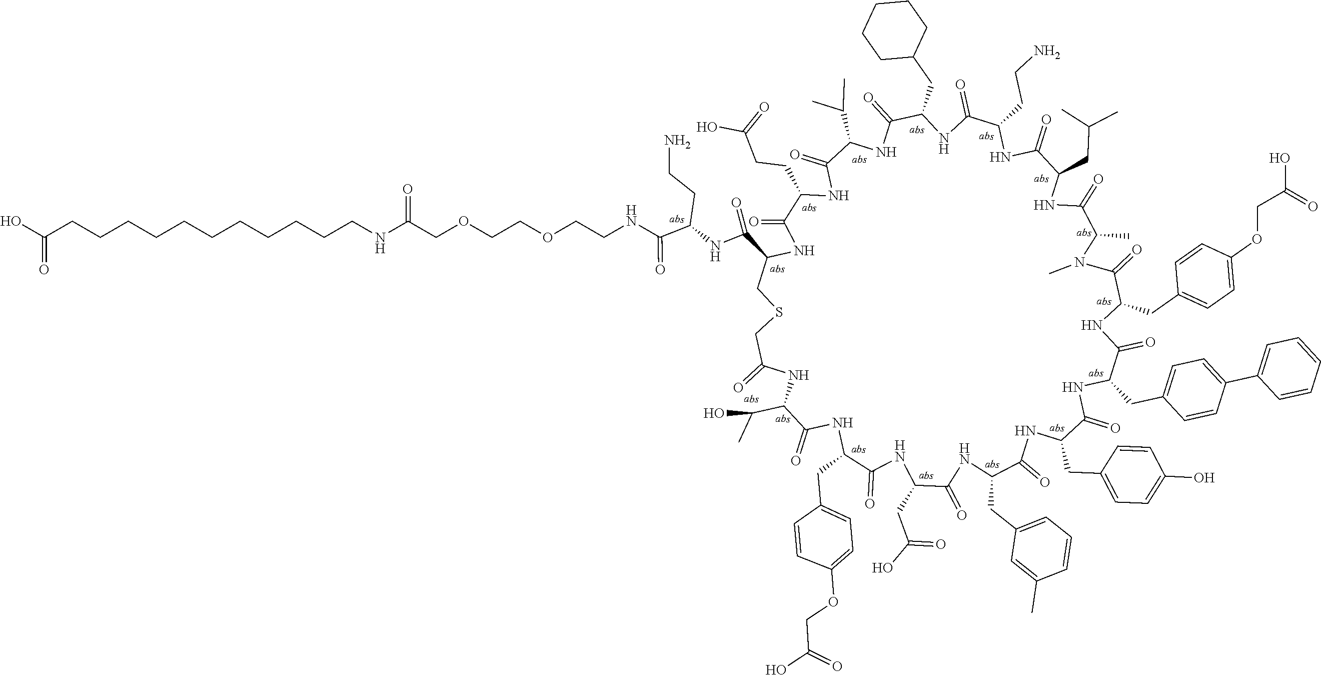 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Example 2666 was prepared on a 100 μmol scale. The yield of the product was 78.1 mg, and its estimated purity by LCMS analysis was 92.5%. Analysis condition A: Retention time=1.54 min; ESI-MS(+) m/z [M+3H]³⁺: 830.5.

### Preparation of Example 2667

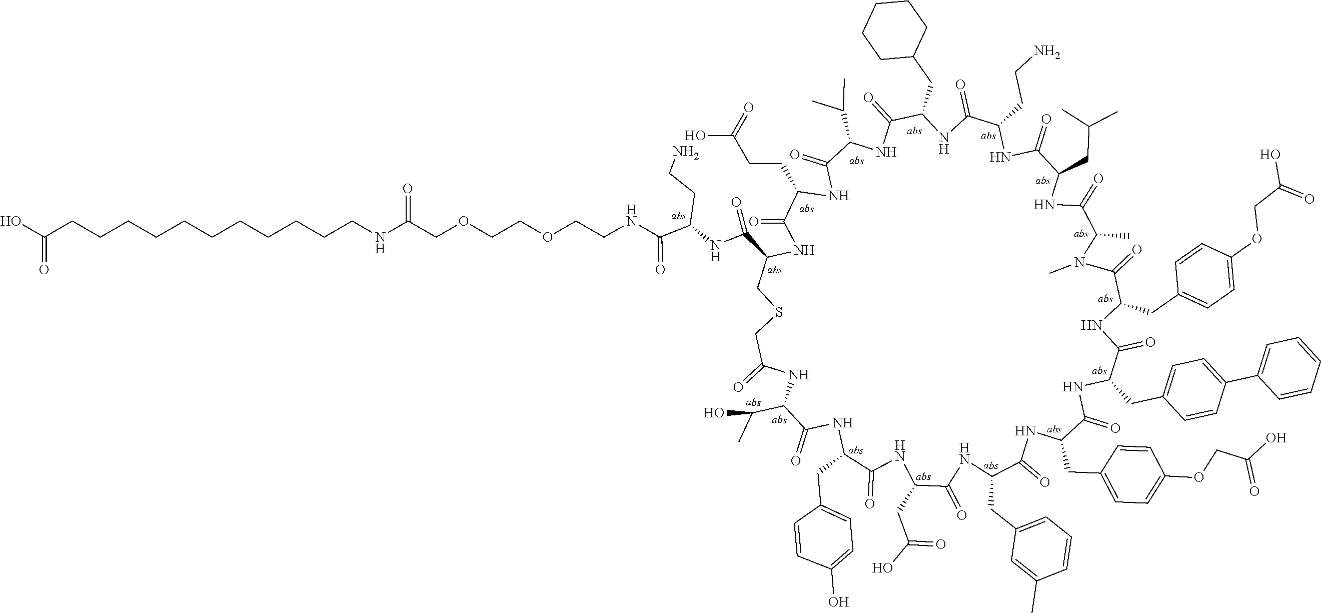 [SMILES: Cc1cccc(C[C@@H]2NC(=O)[C@H](CC(=O)O)NC(=O)[C@H](Cc3ccc(O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2667 was prepared on a 100 μmol scale. The yield of the product was 71.2 mg, and its estimated purity by LCMS analysis was 85.5%. Analysis condition A: Retention time=1.64 min; ESI-MS(+) m/z [M+2H]²⁺: 1245.2.

### Preparation of Example 2668

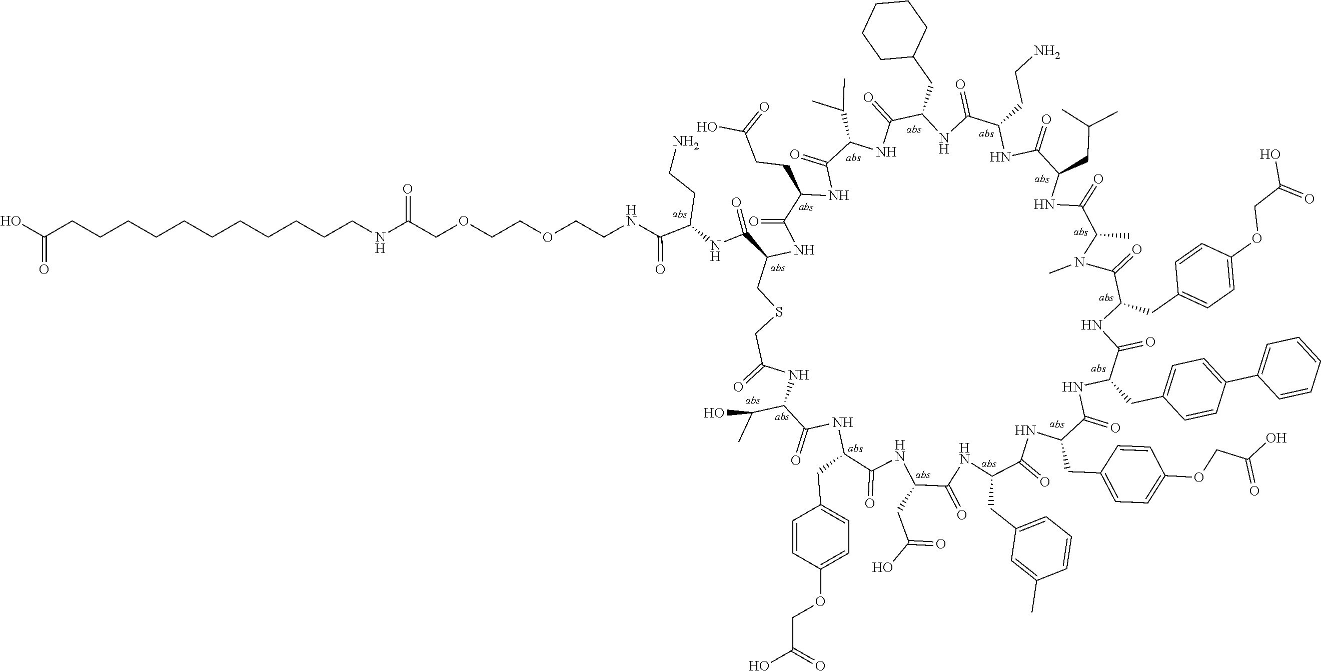 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2668 was prepared on a 100 μmol scale. The yield of the product was 19.2 mg, and its estimated purity by LCMS analysis was 98.9%. Analysis condition B: Retention time=1.97 min; ESI-MS(+) m/z [M+2H]²⁺: 1274.3.

### Preparation of Example 2669

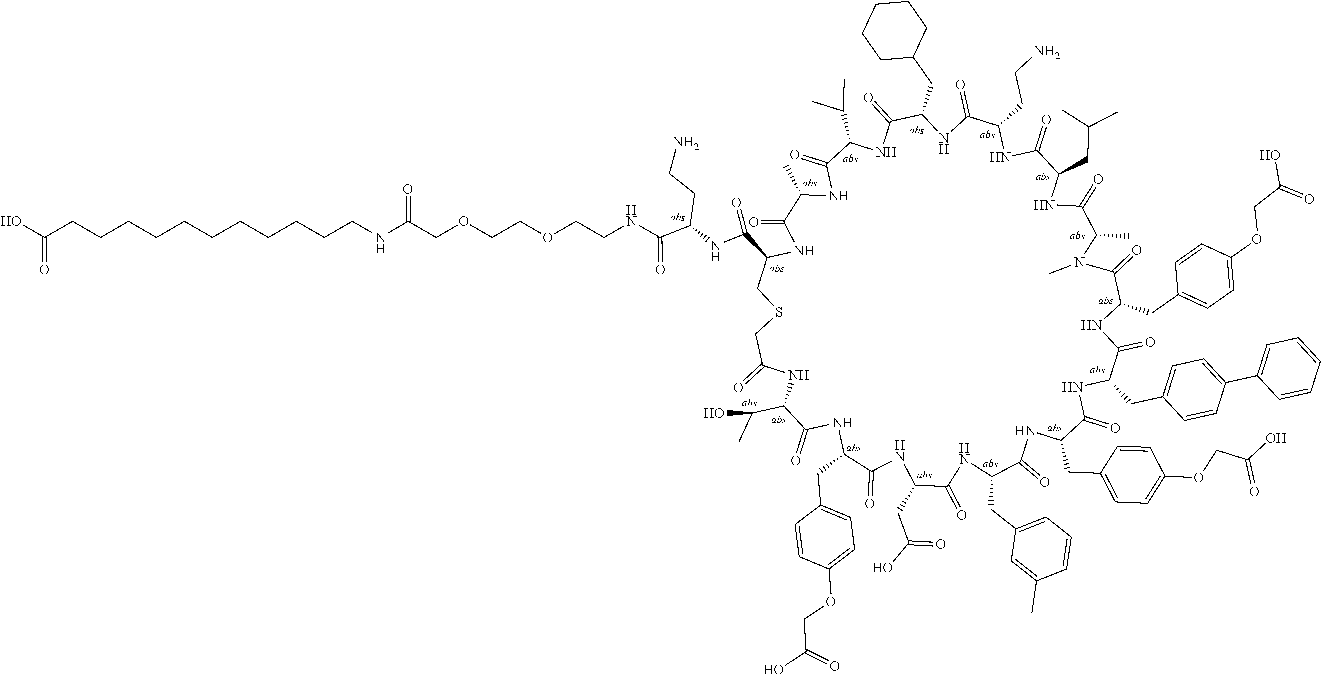 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2669 was prepared on a 100 μmol scale. The yield of the product was 53 mg, and its estimated purity by LCMS analysis was 96%. Analysis condition A: Retention time=1.44 min; ESI-MS(+) m/z [M+2H]²⁺: 1245.2.

### Preparation of Example 2670

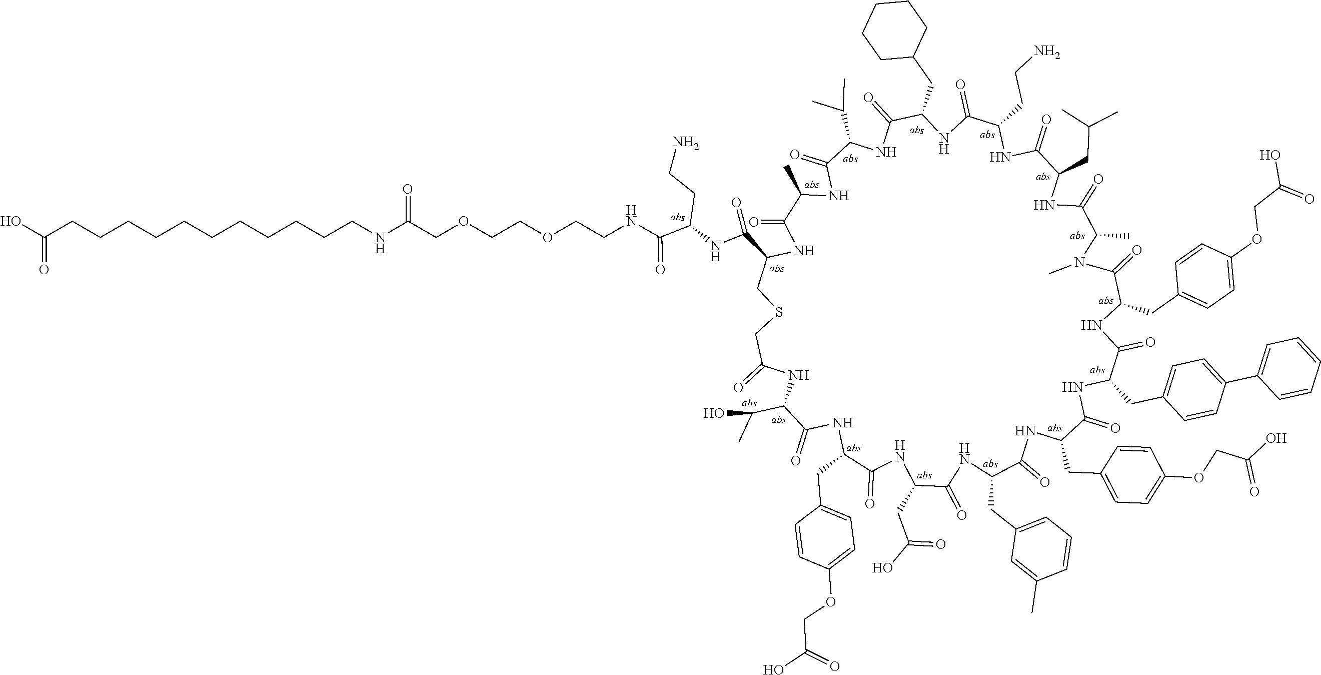 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@@H](C)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2670 was prepared on a 100 μmol scale. The yield of the product was 91.5 mg, and its estimated purity by LCMS analysis was 86.2%. Analysis condition B: Retention time=1.85 min; ESI-MS(+) m/z [M+3H]³⁺: 830.7.

### Preparation of Example 2671

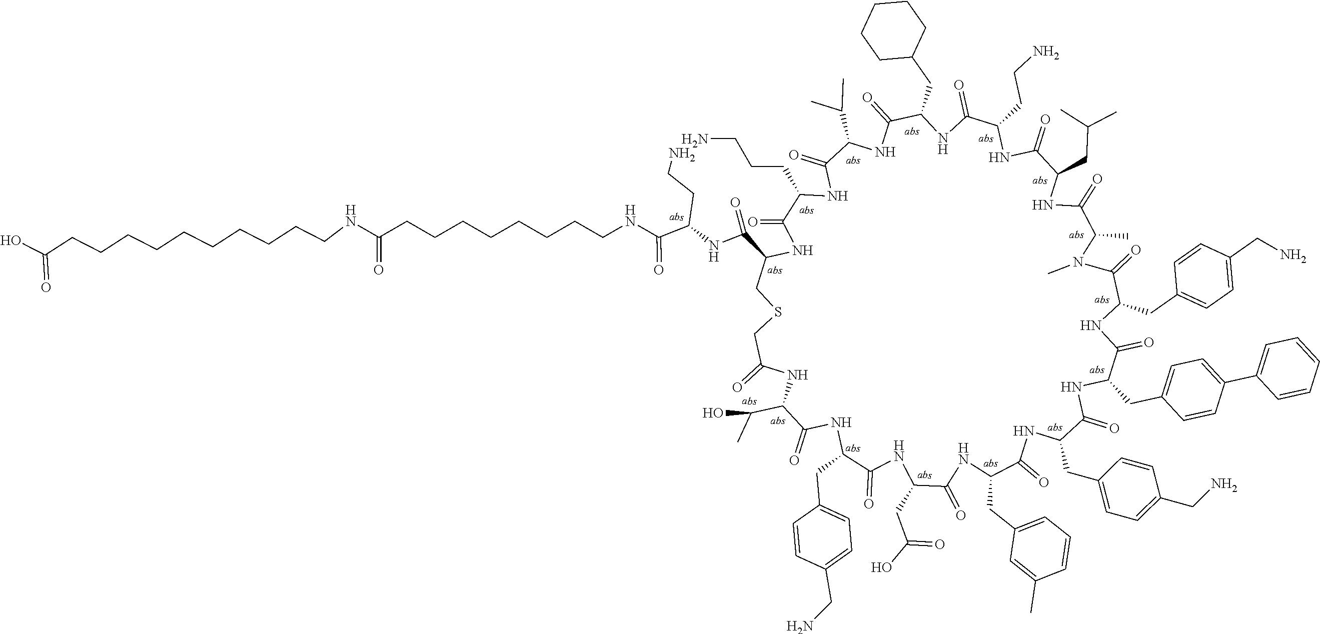 [SMILES: Cc1cccc(C[C@@H]2NC(=O)[C@H](CC(=O)O)NC(=O)[C@H](Cc3ccc(CN)cc3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(CN)cc3)NC(=O)[C@H](Cc3ccc(-c4ccccc4)cc3)NC(=O)[C@H](Cc3ccc(CN)cc3)NC2=O)c1]

Example 2671 was prepared on a 100 μmol scale. The yield of the product was 16.7 mg, and its estimated purity by LCMS analysis was 88.3%. Analysis condition A: Retention time=1.53 min; ESI-MS(+) m/z [M+3H]³⁺: 798.8.

### Preparation of Example 2672

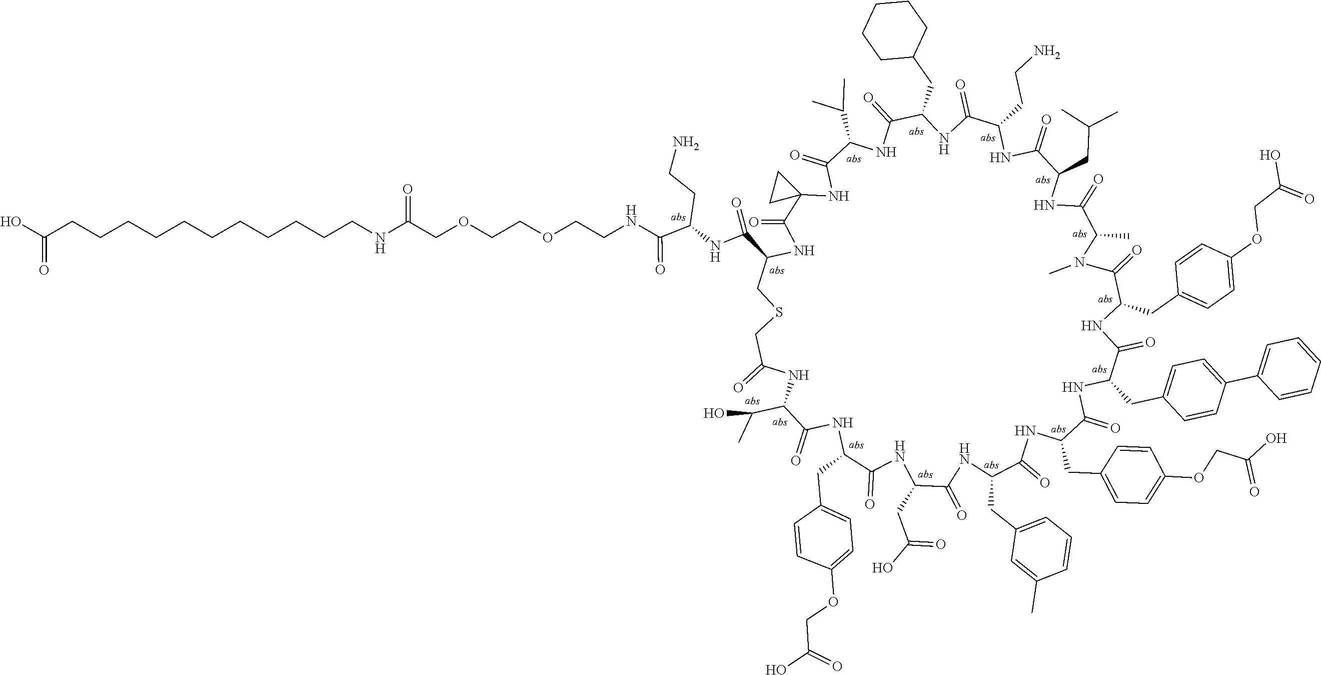 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)C3(CC3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2672 was prepared on a 100 μmol scale. The yield of the product was 49.9 mg, and its estimated purity by LCMS analysis was 92.4%. Analysis condition A: Retention time=1.55 min; ESI-MS(+) m/z [M+3H]³⁺: 834.6.

### Preparation of Example 2673

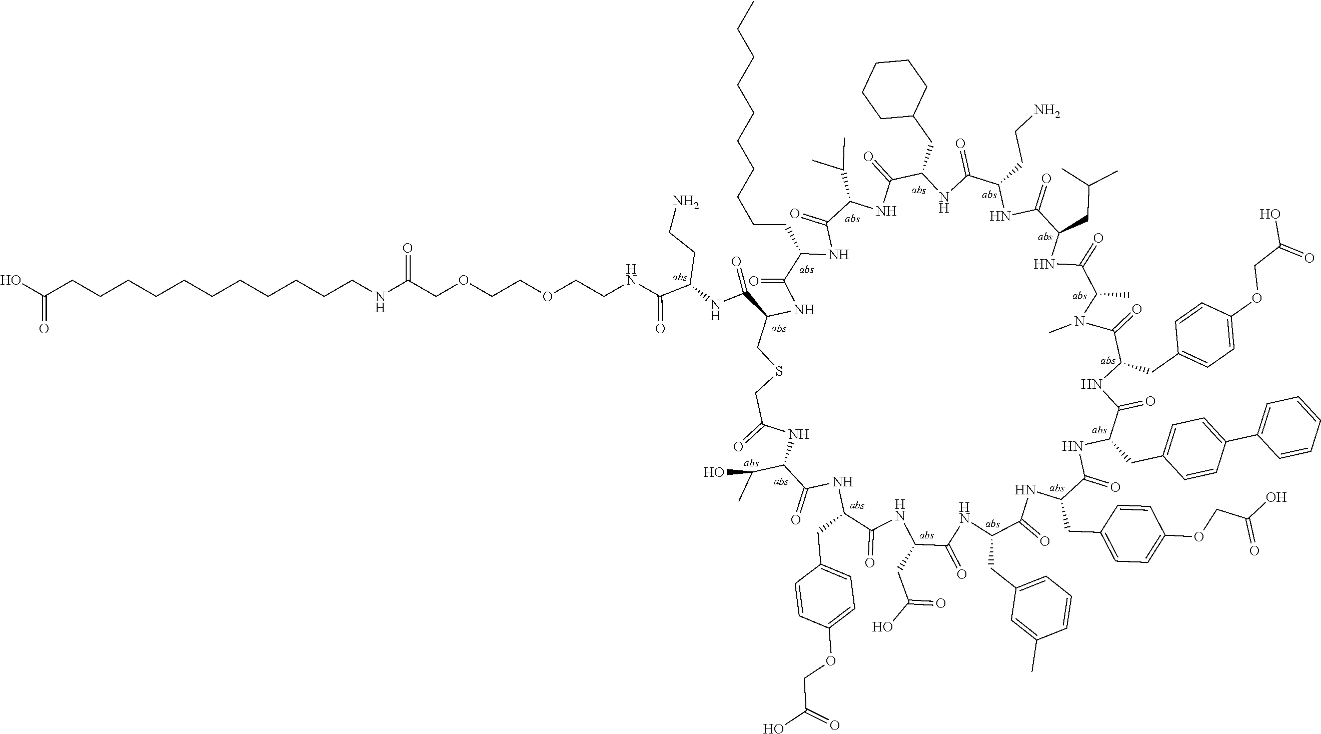 [SMILES: CCCCCCCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](C)N(C)C(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC1=O]

Example 2673 was prepared on a 100 μmol scale. The yield of the product was 27.7 mg, and its estimated purity by LCMS analysis was 95%. Analysis condition B: Retention time=2.26 min; ESI-MS(+) m/z [M+3H]³⁺: 872.8.

### Preparation of Example 2674

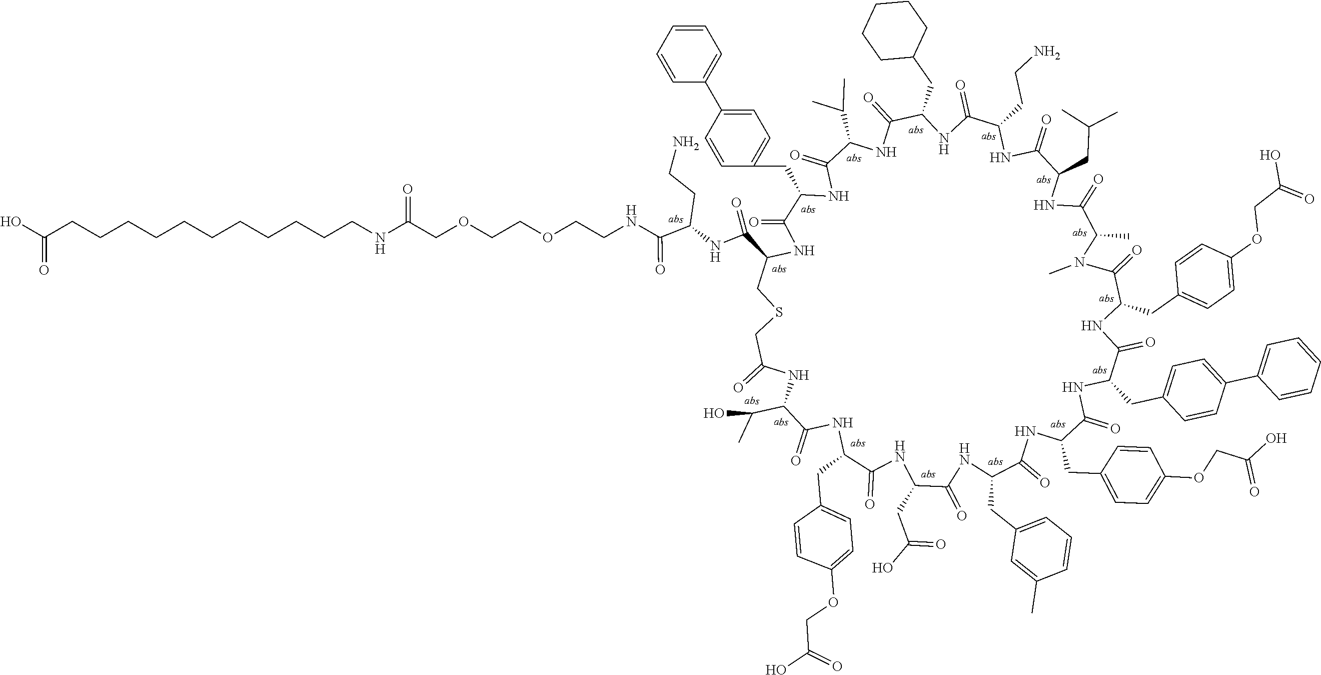 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](Cc3ccc(-c4ccccc4)cc3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2674 was prepared on a 100 μmol scale. The yield of the product was 91.5 mg, and its estimated purity by LCMS analysis was 95.2%. Analysis condition A: Retention time=1.69 min; ESI-MS(+) m/z [M+3H]³⁺: 881.2.

### Preparation of Example 2675

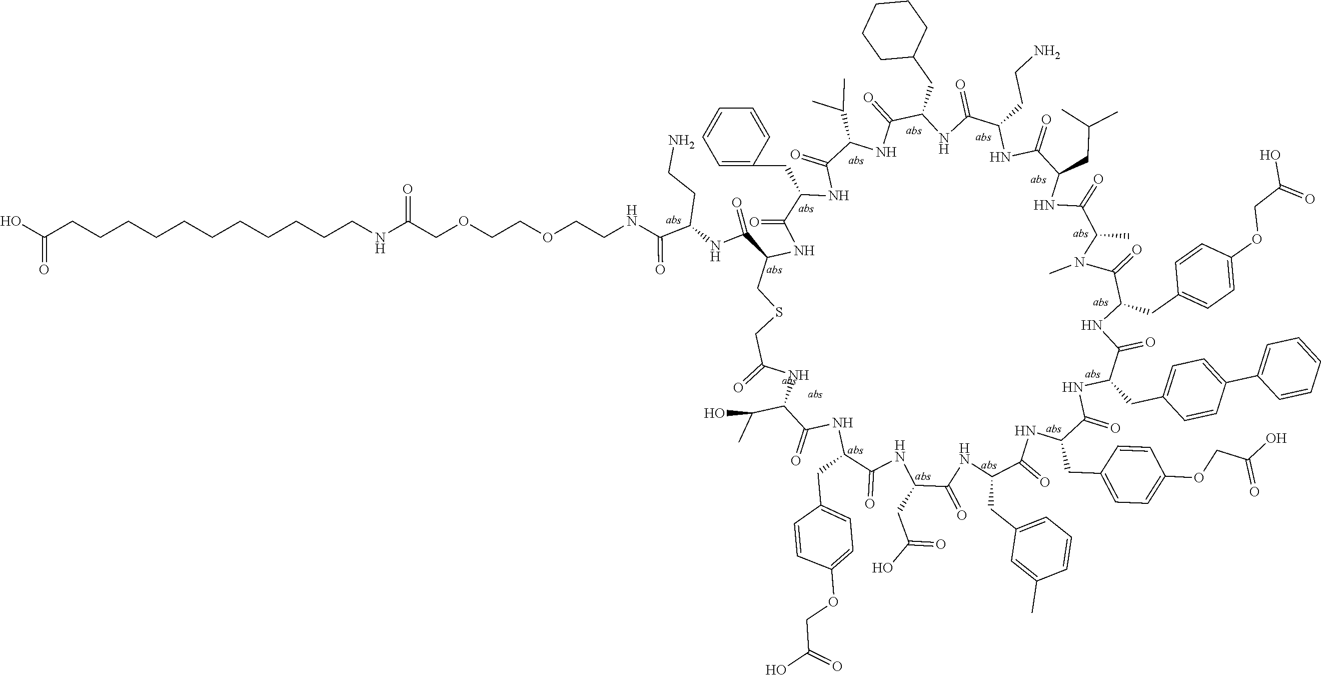 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCC(=O)O)NC(=O)[C@H](Cc3ccccc3)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2675 was prepared on a 100 μmol scale. The yield of the product was 16.1 mg, and its estimated purity by LCMS analysis was 86.9%. Analysis condition A: Retention time=1.74 min; ESI-MS(+) m/z [M+3H]³⁺: 881.2.

### Preparation of Example 2676

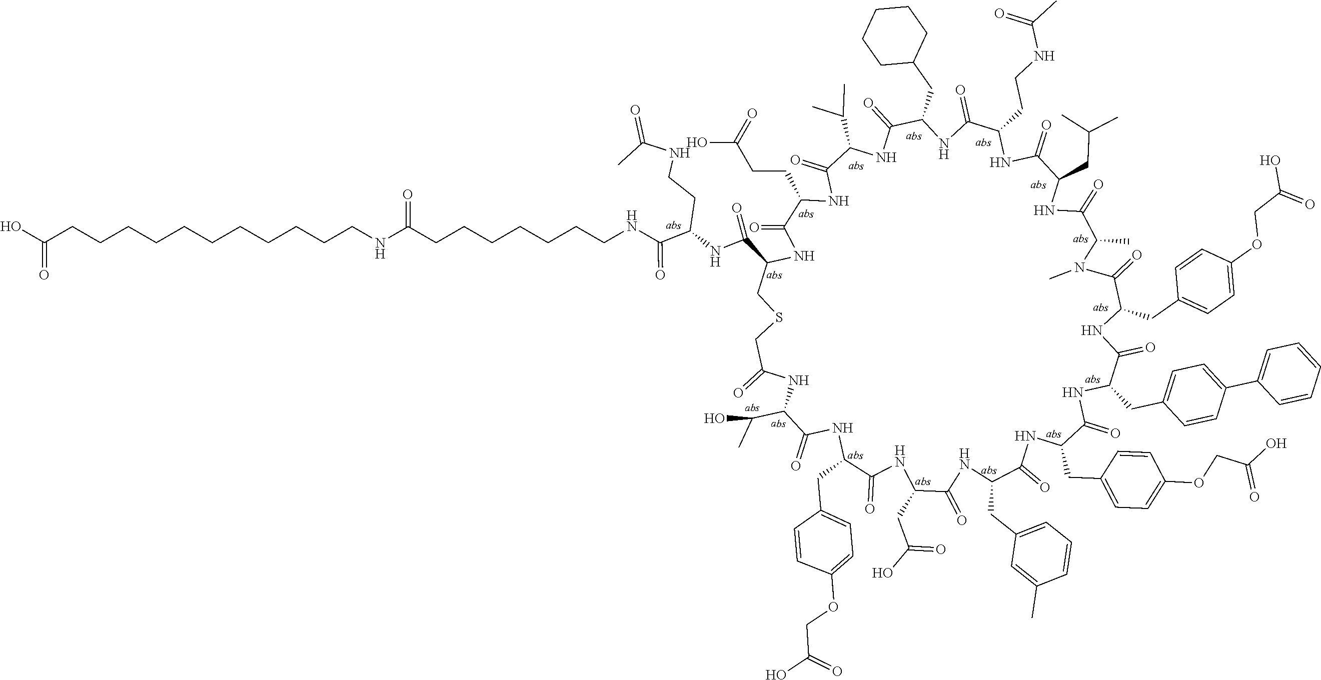 [SMILES: CC(=O)NCC[C@H](NC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N(C)[C@@H](C)C(=O)N[C@H](CC(C)C)C(=O)N[C@@H](CCNC(C)=O)C(=O)N[C@@H](CC2CCCCC2)C(=O)N[C@@H](C(C)C)C(=O)N[C@@H](CCC(=O)O)C(=O)N1)C(=O)NCCCCCCCC(=O)NCCCCCCCCCCCC(=O)O]

Example 2676 was prepared on a 3.2 μmol scale. The yield of the product was 0.7 mg, and its estimated purity by LCMS analysis was %. Analysis condition B: Retention time=2.06 min; ESI-MS(+) m/z [M+3H]³⁺: 876.4.

### Preparation of Example 2677

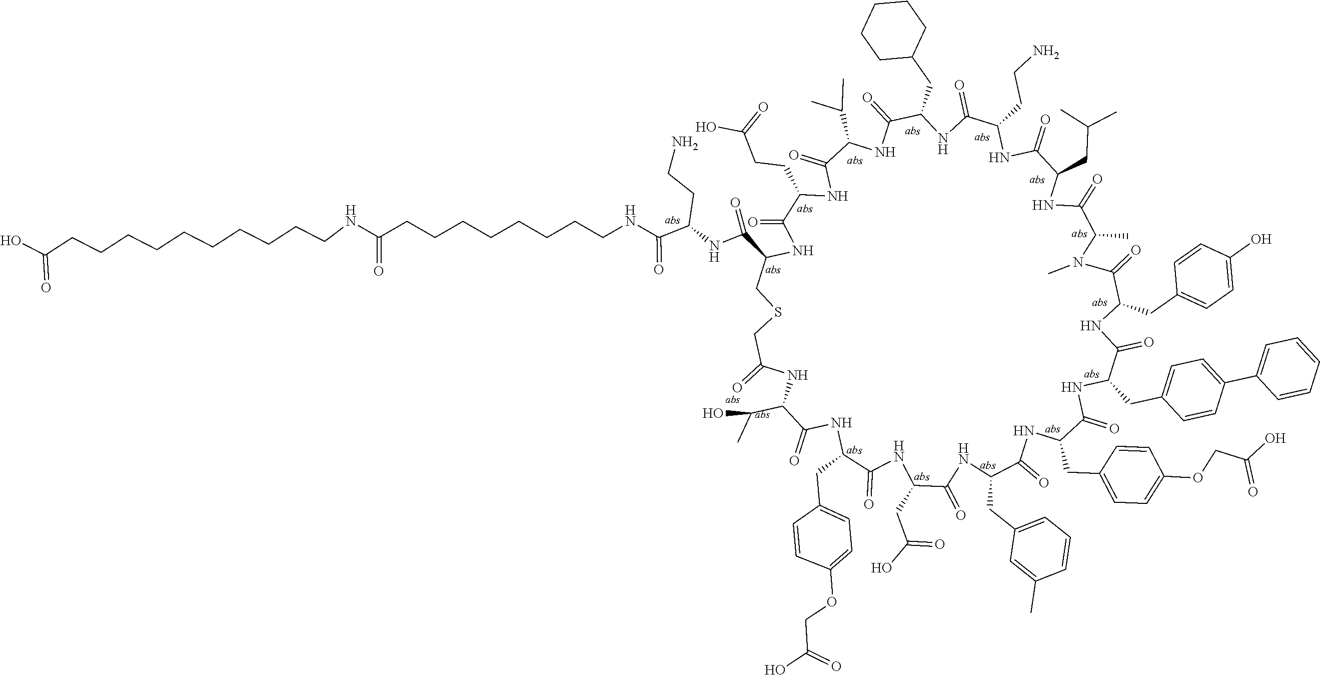 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2677 was prepared on a 100 μmol scale. The yield of the product was 3.5 mg, and its estimated purity by LCMS analysis was 94.7%. Analysis condition A: Retention time=1.54 min; ESI-MS(+) m/z [M+2H]²⁺: 1272.5.

### Preparation of Example 2678

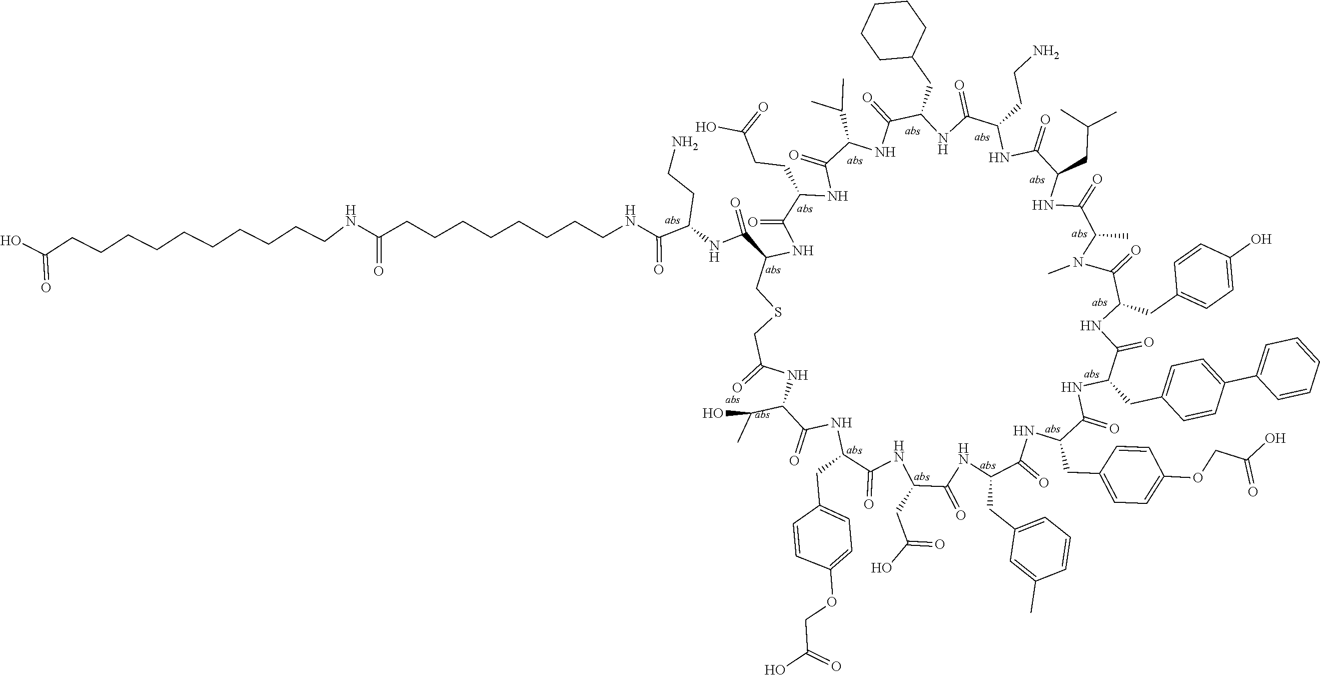 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2678 was prepared on a 100 μmol scale. The yield of the product was 38.9 mg, and its estimated purity by LCMS analysis was 94%. Analysis condition A: Retention time=1.57 min; ESI-MS(+) m/z [M+2H]²⁺: 1286.9.

### Preparation of Example 2679

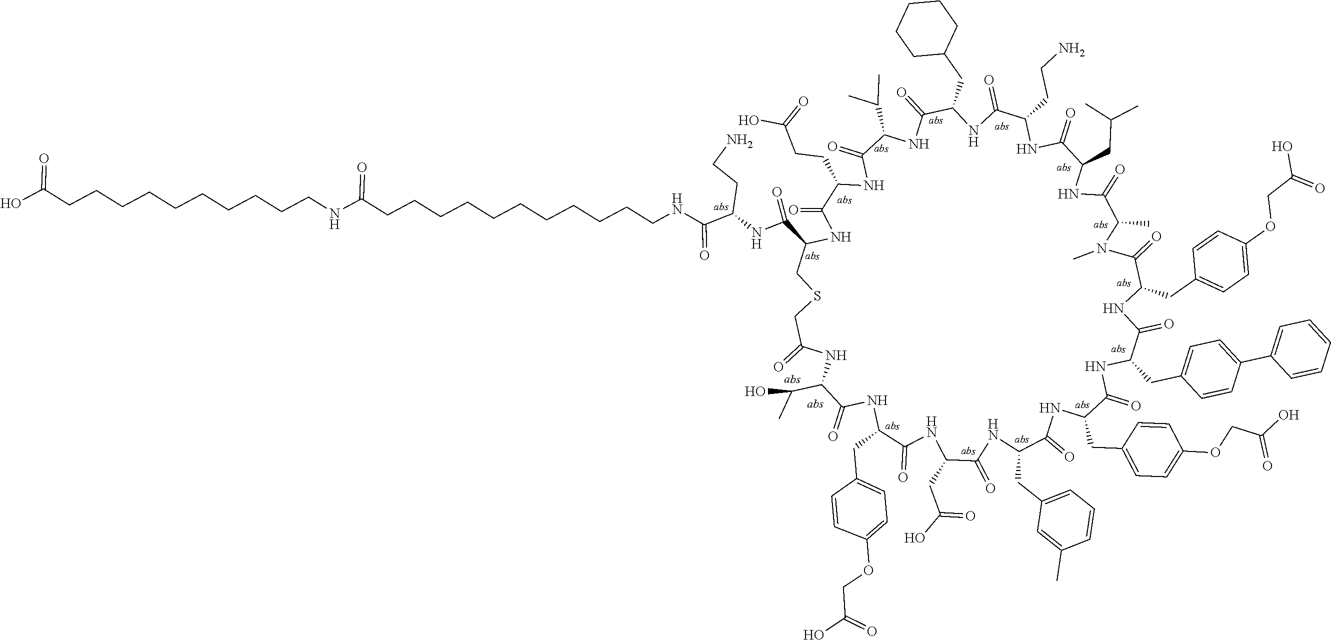 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2679 was prepared on a 100 μmol scale. The yield of the product was 3.8 mg, and its estimated purity by L CMS analysis was 100%. Analysis condition A: Retention time=1.62 min; ESI-MS(+) m/z [M+2H]²⁺: 1293.2.

### Preparation of Example 2680

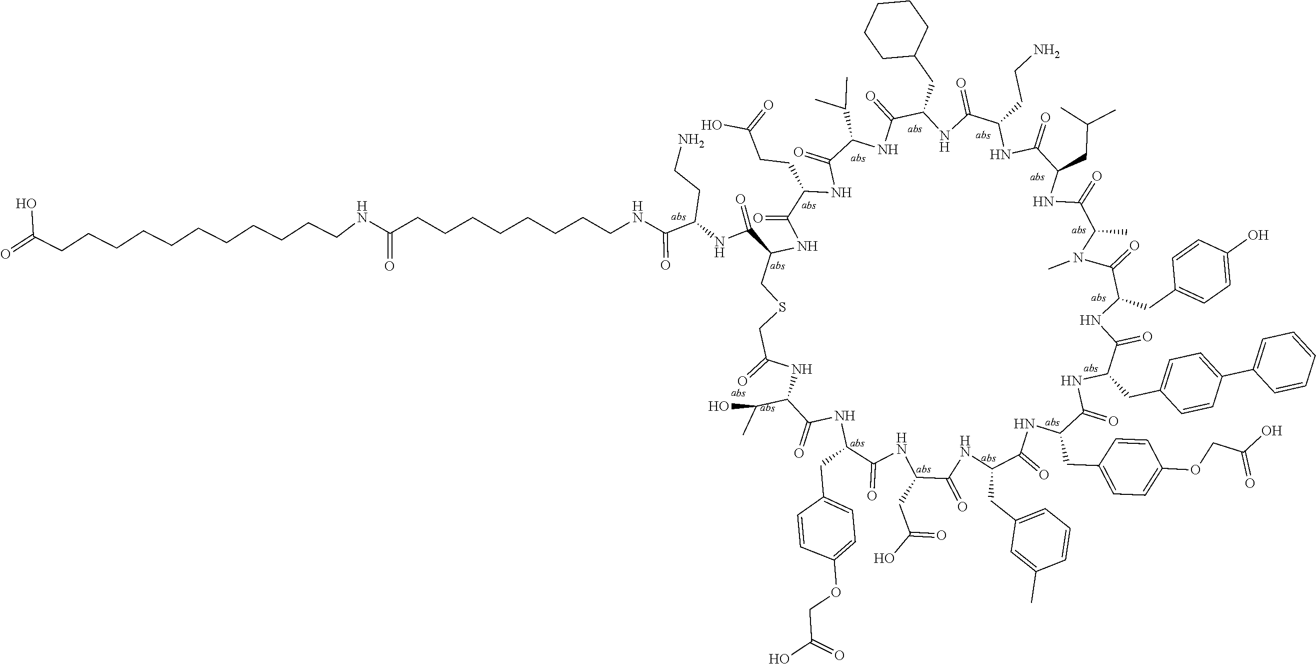 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2680 was prepared on a 100 μmol scale. The yield of the product was 28.4 mg, and its estimated purity by LCMS analysis was 90.9%. Analysis condition A: Retention time=1.55 min; ESI-MS(+) m/z [M+2H]²⁺: 1279.7.

### Preparation of Example 2681

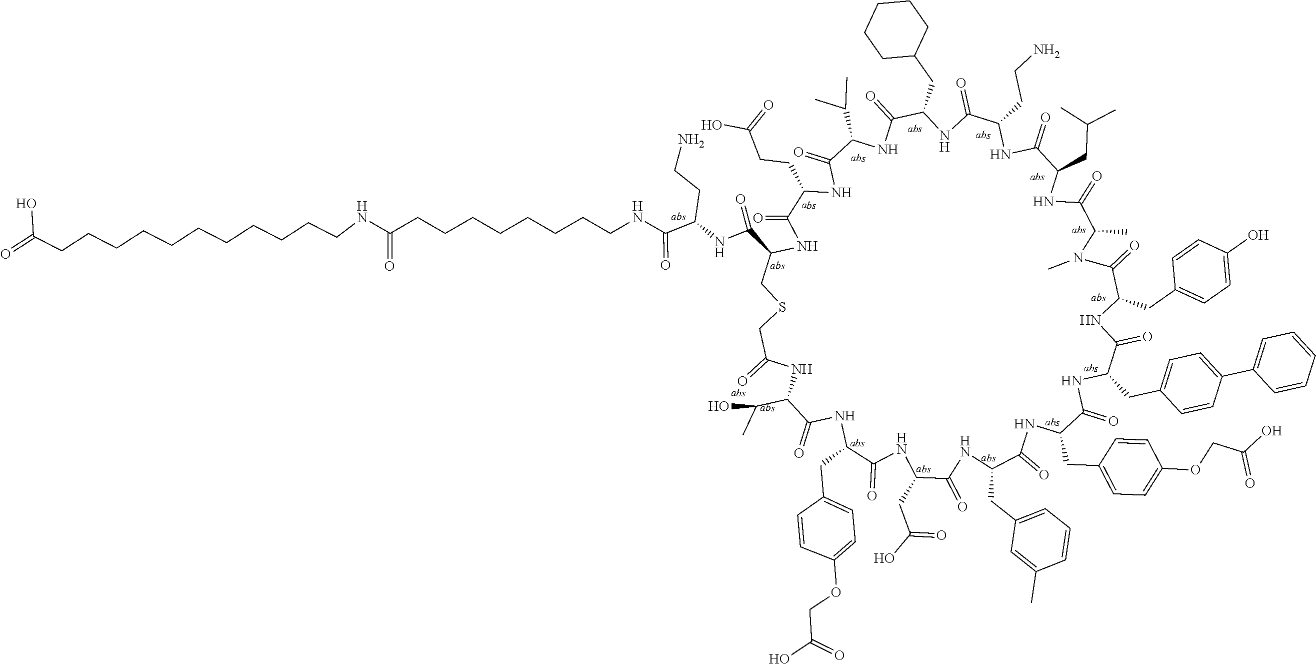 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2681 was prepared on a 100 μmol scale. The yield of the product was 49.7 mg, and its estimated purity by LCMS analysis was 95.5%. Analysis condition A: Retention time=1.62 min; ESI-MS(+) m/z [M+2H]²⁺: 1294.

### Preparation of Example 2682

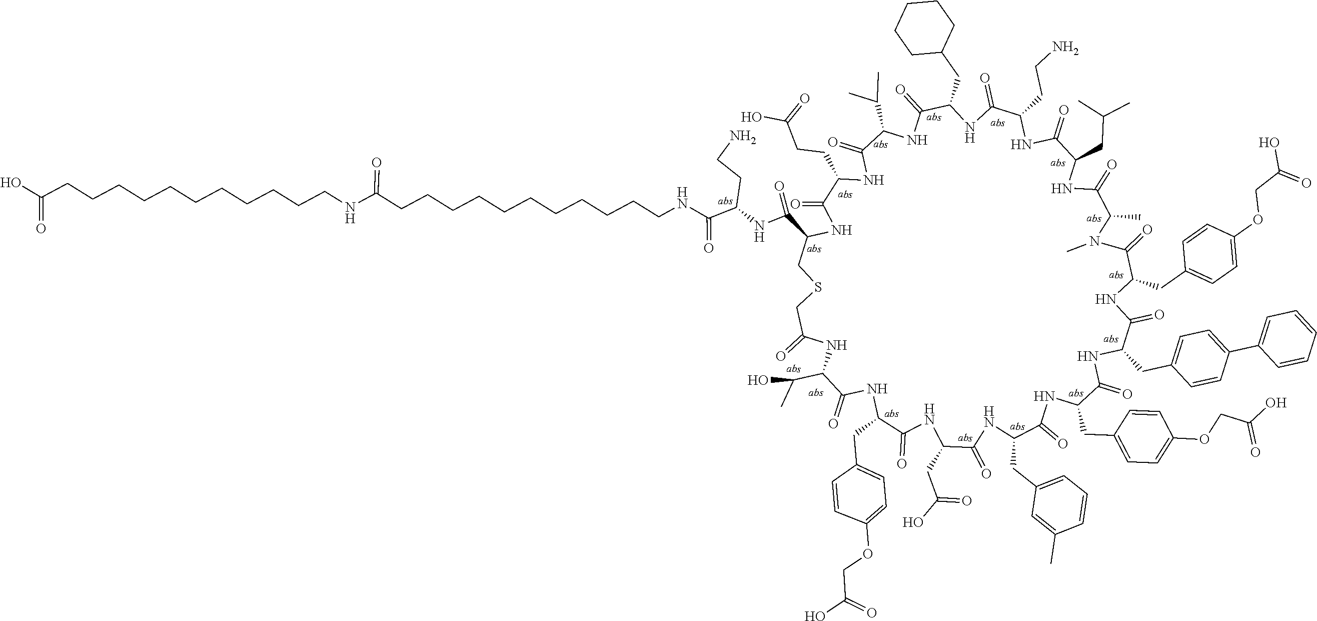 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCCC(=O)NCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2682 was prepared on a 100 μmol scale. The yield of the product was 49.5 mg, and its estimated purity by LCMS analysis was 95.6%. Analysis condition A: Retention time=1.66 min; ESI-MS(+) m/z [M+2H]²⁺: 1300.1.

### Preparation of Example 2683

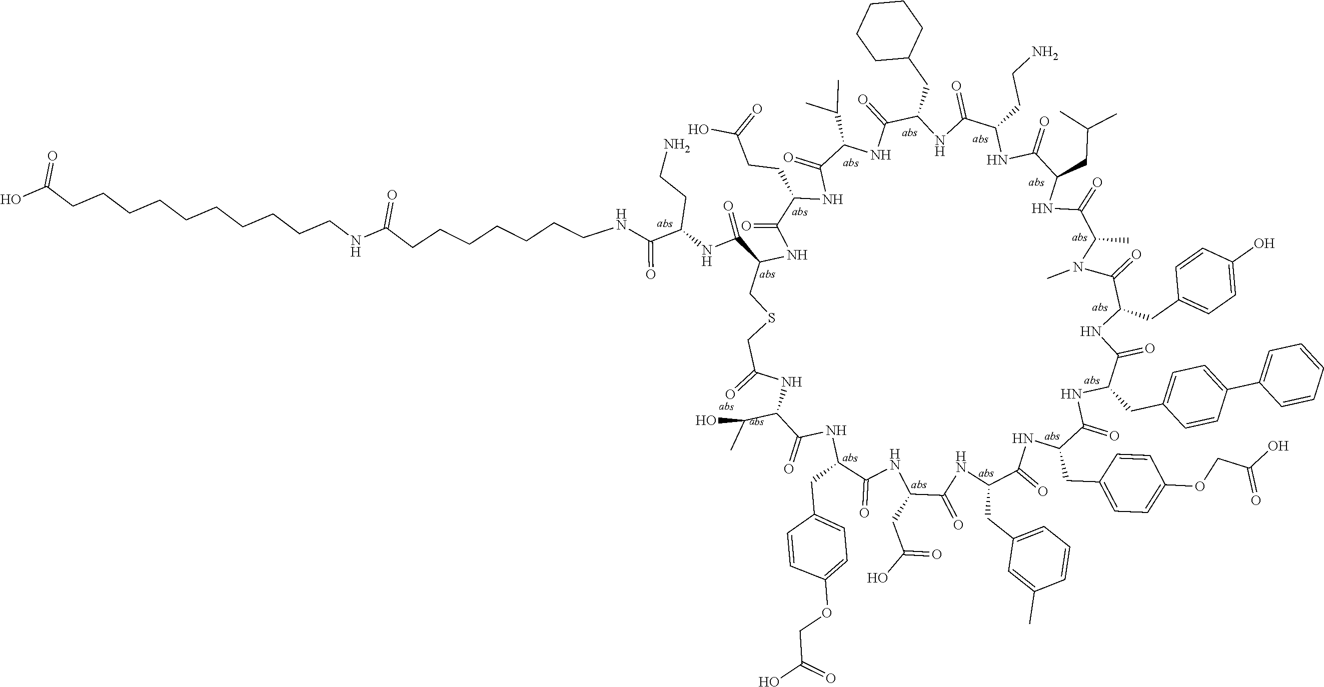 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2683 was prepared on a 100 μmol scale. The yield of the product was 49.7 mg, and its estimated purity by LCMS analysis was 95.7%. Analysis condition A: Retention time=1.51 min; ESI-MS(+) m/z [M+2H]²⁺: 1229.7.

### Preparation of Example 2684

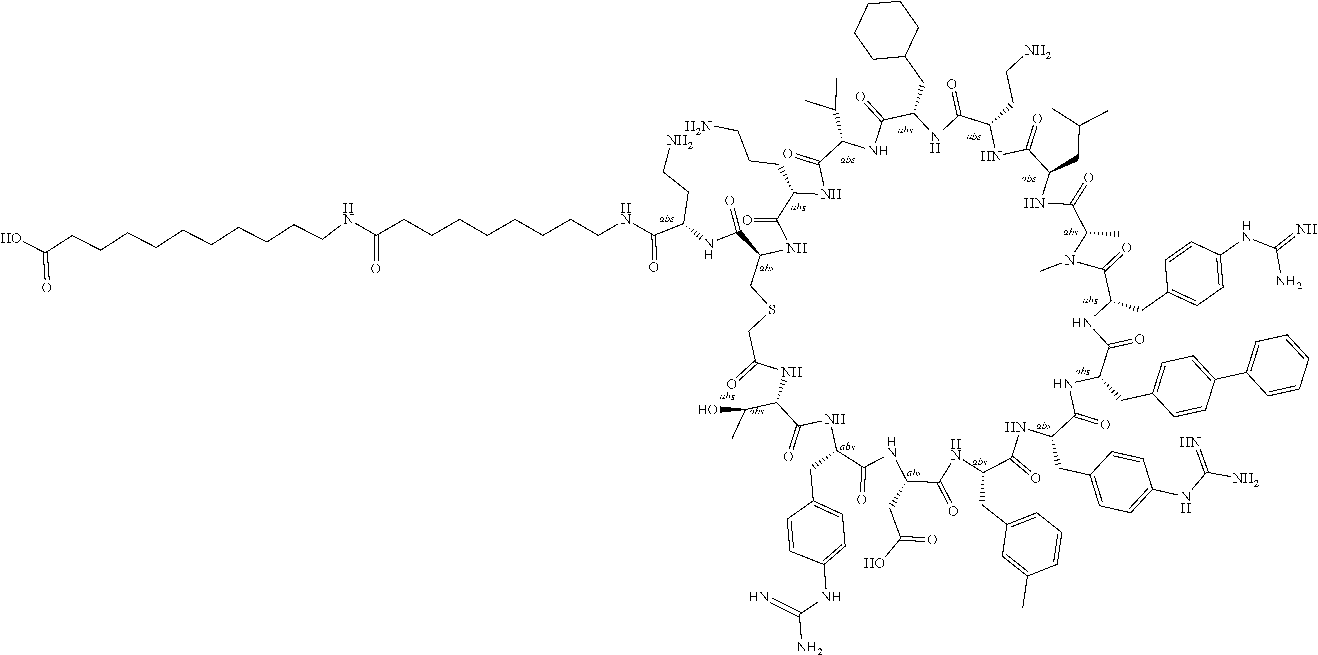 [SMILES: Cc1cccc(C[C@@H]2NC(=O)[C@H](CC(=O)O)NC(=O)[C@H](Cc3ccc(NC(=N)N)cc3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(NC(=N)N)cc3)NC(=O)[C@H](Cc3ccc(-c4ccccc4)cc3)NC(=O)[C@H](Cc3ccc(NC(=N)N)cc3)NC2=O)c1]

Example 2684 was prepared on a 100 μmol scale. The yield of the product was 8.2 mg, and its estimated purity by LCMS analysis was 90.5%. Analysis condition A: Retention time=1.57 min; ESI-MS(+) m/z [M+H]³⁺: 826.9.

### Preparation of Example 2685

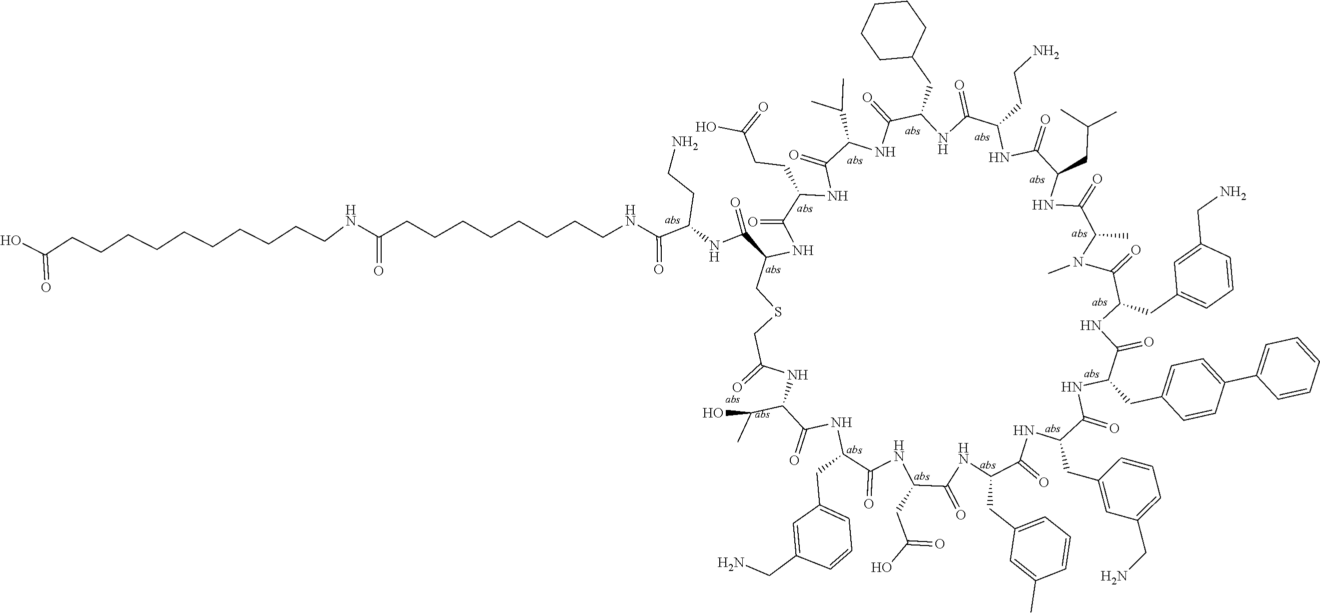 [SMILES: Cc1cccc(C[C@@H]2NC(=O)[C@H](CC(=O)O)NC(=O)[C@H](Cc3cccc(CN)c3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3cccc(CN)c3)NC(=O)[C@H](Cc3ccc(-c4ccccc4)cc3)NC(=O)[C@H](Cc3cccc(CN)c3)NC2=O)c1]

Example 2685 was prepared on a 100 μmol scale. The yield of the product was 18.5 mg, and its estimated purity by LCMS analysis was 94.8%. Analysis condition A: Retention time=1.56 min; ESI-MS(+) m/z [M+2H]²⁺: 1197.60.

### Preparation of Example 2686

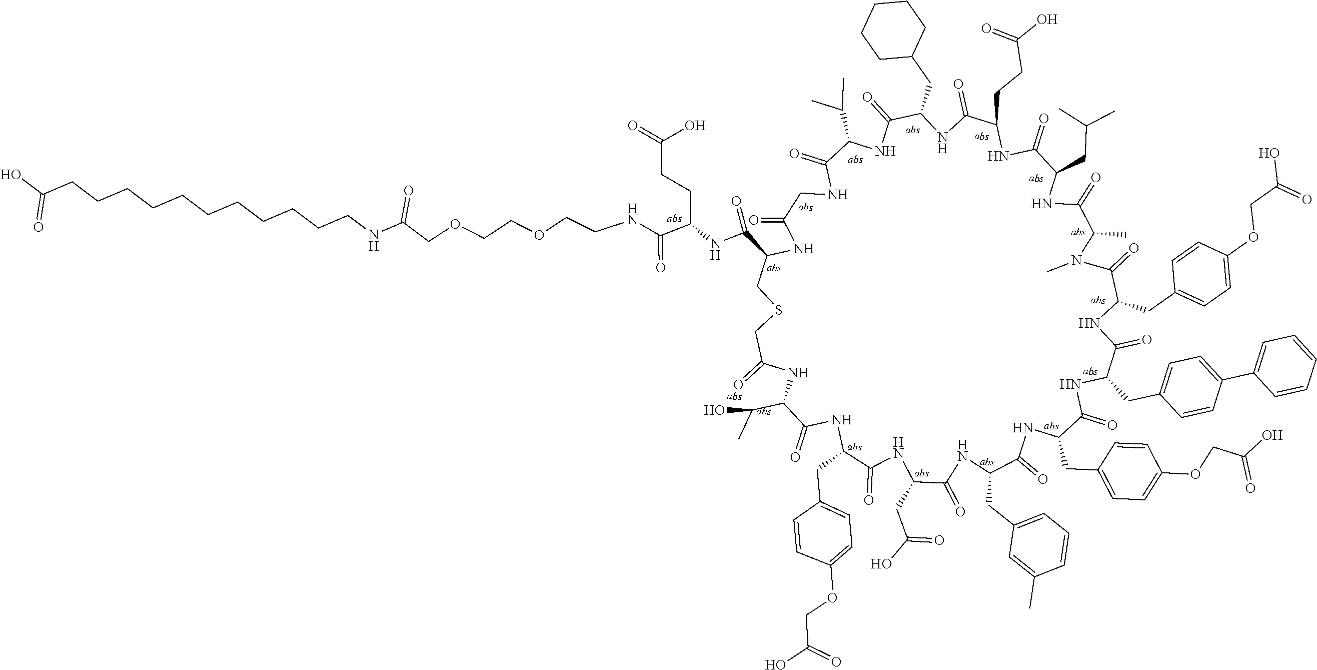 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCC(=O)O)C(=O)NCCOCCOCC(=O)NCCCCCCCCCCCC(=O)O)NC(=O)CNC(=O)[C@H](C(C)C)NC(=O)[C@H](CC3CCCCC3)NC(=O)[C@@H](CCC(=O)O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2686 was prepared on a 100 μmol scale. The yield of the product was 64.7 mg, and its estimated purity by LCMS analysis was 89.4%. Analysis condition A: Retention time=1.37 min; ESI-MS(+) m/z [M+2H]²⁺: 1267.9.

### Preparation of Example 2687

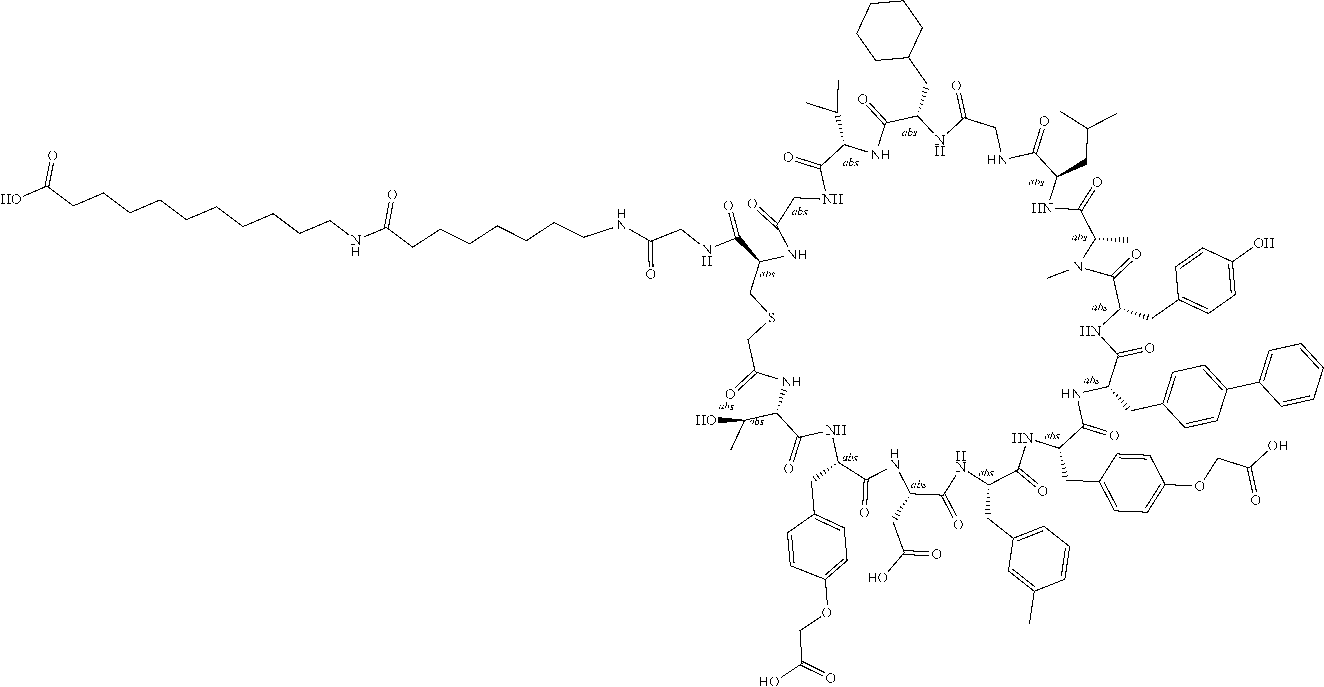 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)NCC(=O)NCCCCCCCC(=O)NCCCCCCCCCCC(=O)O)NC(=O)CNC(=O)[C@H](C(C)C)NC(=O)[C@H](CC3CCCCC3)NC(=O)CNC(=O)[C@@H](CC(C)C)NC(=O)[C@H](C)N(C)C(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Example 2687 was prepared on a 100 μmol scale. The yield of the product was 57.8 mg, and its estimated purity by LCMS analysis was 98%. Analysis condition A: Retention time=1.51 min; ESI-MS(+) m/z [M+2H]²⁺: 1186.1.

### Preparation of Example 2688

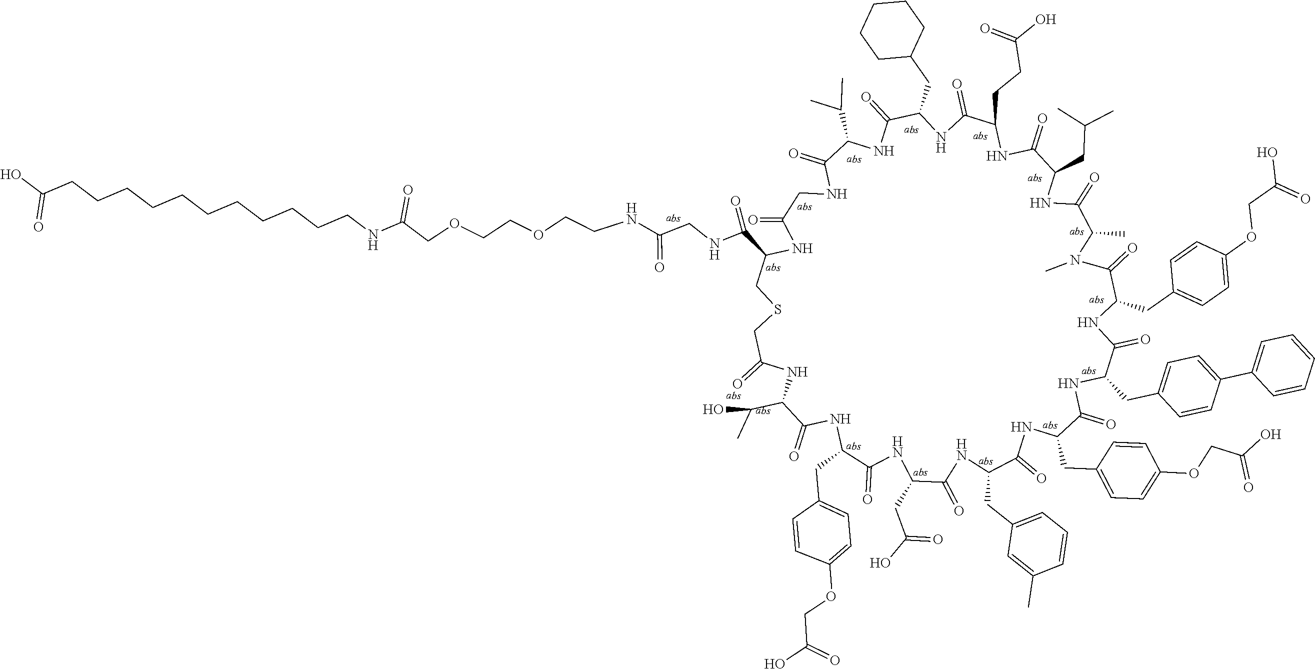 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)NCC(=O)NCCOCCOCC(=O)NCCCCCCCCCCCC(=O)O)NC(=O)CNC(=O)[C@H](C(C)C)NC(=O)[C@H](CC3CCCCC3)NC(=O)[C@@H](CCC(=O)O)NC(=O)[C@@H](CC(C)C)NC(=O)[C@H](C)N(C)C(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Example 2688 was prepared on a 100 μmol scale. The yield of the product was 104.1 mg, and its estimated purity by LCMS analysis was 92.4%. Analysis condition A: Retention time=1.36 min; ESI-MS(+) m/z [M+2H]²⁺: 1231.1.

### Preparation of Example 2689

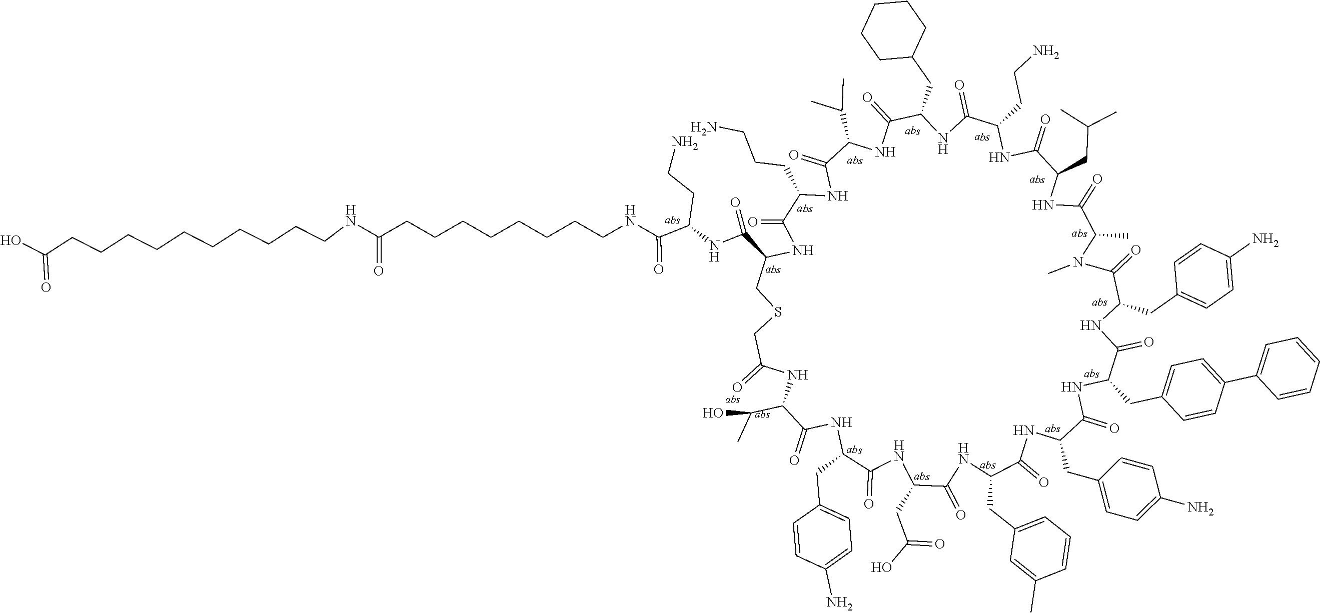 [SMILES: Cc1cccc(C[C@@H]2NC(=O)[C@H](CC(=O)O)NC(=O)[C@H](Cc3ccc(N)cc3)NC(=O)[C@H]([C@@H](C)O)NC(=O)CSC[C@@H](C(=O)N[C@@H](CCN)C(=O)NCCCCCCCCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](Cc3ccc(N)cc3)NC(=O)[C@H](Cc3ccc(-c4ccccc4)cc3)NC(=O)[C@H](Cc3ccc(N)cc3)NC2=O)c1]

Example 2689 was prepared on a 100 μmol scale. The yield of the product was 25.4 mg, and its estimated purity by LCMS analysis was 95.100. Analysis condition A: Retention time=1.79 min; ESI-MS(+) m/z [M+2H]²⁺: 1176.6.

### Example 2: Jurkat-PD-1 Cell Binding High-Content Screening Assay (CBA)

Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×10³ Jurkat-hPD-1 cells were seeded into 384 well plates in 20 μl of DMEM supplemented with 10% fetal calf serum. 100 nl of compound was added to cells followed by incubation at 37° C. for 2 hours. Then, 5 μl of PE-labeled PD-L1-Ig (20 nM final), diluted in DMEM supplemented with 10% fetal calf serum. After 1 hour incubation, cells were fixed with 400 paraformaldehyde in dPBS containing 10 μg/ml Hoechst 33342 and then washed 3× in 100 μl dPBS. Data was collected and processed using a Cell Insight NXT High Content Imager and associated software.

### Protein Sequence Information

| hPDL1(18-239)-TVMV-mIgG1(221-447)-C225S | |
|---|---|
| (SEQ ID NO: 1) | |
| 1 | AFTVTVPKDL YVVEYGSNMT IECKFPVEKQ LDLAALIVYW EMEDKNIIQF |
| 51 | VHGEEDLKVQ HSSYRQRARL LKDQLSLGNA ALQITDVKLQ DAGVYRCMIS |
| 101 | YGGADYKRIT VKVNAPYNKI NQRILVVDPV TSEHELTCQA EGYPKAEVIW |
| 151 | TSSDHQVLSG KTTTTNSKRE EKLFNVTSTL RINTTTNEIF YCTFRRLDPE |
| 201 | ENHTAELVIP ELPLAHPPNE RTGSPGGGGG RETVRFQGGT GDAVPRDSGC |
| 251 | KPCICTVPEV SSVFIFPPKP KDVLTITLTP KVTCVVVDIS KDDPEVQFSW |
| 301 | FVDDVEVHTA QTQPREEQFN STFRSVSELP IMHQDWLNGK EFKCRVNSAA |
| 351 | FPAPIEKTIS KTKGRPKAPQ VYTIPPPKEQ MAKDKVSLTC MITDFFPEDI |
| 401 | TVEWQWNGQP AENYKNTQPI MDTDGSYFVY SKLNVQKSNW EAGNTFTCSV |
| 451 | LHEGLHNHHT EKSLSHSPGK |

Jurkat HPDL1 PD1 IC₅₀ (μM) is presented in Table 1.

TABLE 1

| | Jurkat | | Jurkat | | Jurkat |
|---|---|---|---|---|---|
| | HPD1 | | HPD1 | | HPD1 |
| | PDL1 | | PDL1 | | PDL1 |
| Example | IC50 | Example | IC50 | Example | IC50 |
| Number | (uM) | Number | (uM) | Number | (uM) |
| 1000 | 0.0234 | 1022 | 0.0766 | 1045 | 0.0149 |
| 1001 | 0.0921 | 1023 | 0.1189 | 1046 | 0.0124 |
| 1002 | 0.0110 | 1024 | 0.0875 | 1047 | 0.0296 |
| 1002 | 0.0110 | 1025 | 0.1121 | 1048 | 0.0165 |
| 1003 | 0.0201 | 1026 | 0.0805 | 1049 | 0.0215 |
| 1004 | 0.0513 | 1027 | 0.0219 | 1050 | 0.0217 |
| 1005 | 0.0495 | 1028 | 0.0147 | 1051 | 0.0298 |
| 1006 | 0.0181 | 1029 | 0.0090 | 1052 | 0.0184 |
| 1007 | 0.1115 | 1030 | 0.0106 | 1053 | 0.0231 |
| 1008 | 0.0208 | 1031 | 0.0107 | 1054 | 0.0400 |
| 1009 | 0.0278 | 1032 | 0.0159 | 1055 | 0.0193 |
| 1010 | 0.0283 | 1033 | 0.0205 | 1056 | 0.0387 |
| 1011 | 0.0246 | 1034 | 0.0220 | 1057 | 0.1305 |
| 1012 | 0.0650 | 1035 | 0.0194 | 1058 | 0.0176 |
| 1013 | 0.0365 | 1036 | 0.0115 | 1059 | 0.0585 |
| 1014 | 0.0485 | 1037 | 0.0136 | 1060 | 0.0479 |
| 1015 | 0.0541 | 1038 | 0.0132 | 1061 | 0.0429 |
| 1016 | 0.0298 | 1039 | 0.0128 | 1062 | 0.0269 |
| 1017 | 0.1066 | 1040 | 0.0140 | 1063 | 0.0279 |
| 1018 | 0.0677 | 1041 | 0.0138 | 1064 | 0.0236 |
| 1019 | 0.0585 | 1042 | 0.0098 | 1065 | 0.0324 |
| 1020 | 0.1204 | 1043 | 0.0124 | 1066 | 0.0639 |
| 1021 | 0.0762 | 1044 | 0.0090 | 1067 | 0.1062 |
| 1068 | 0.1717 | 1097 | 0.0071 | 1126 | 0.0034 |
| 1069 | 0.0407 | 1098 | 0.0086 | 1127 | 0.0038 |
| 1070 | 0.0392 | 1099 | 0.0098 | 1128 | 0.0040 |
| 1071 | 0.0254 | 1100 | 0.0198 | 1129 | 0.0067 |
| 1072 | 0.0183 | 1101 | 0.0480 | 1130 | 0.0123 |
| 1073 | 0.0360 | 1102 | 0.0157 | 1131 | 0.6163 |
| 1074 | 0.0434 | 1103 | 0.0190 | 1132 | 0.0268 |
| 1075 | 0.0202 | 1104 | 0.5587 | 1133 | 0.0293 |
| 1076 | 0.0165 | 1105 | 0.0072 | 1134 | 0.0153 |
| 1077 | 0.0183 | 1106 | 0.0853 | 1135 | 0.0125 |
| 1078 | 0.0161 | 1107 | 0.0516 | 1136 | 0.0181 |
| 1079 | 0.0108 | 1108 | 0.0475 | 1137 | 0.0135 |
| 1080 | 0.0186 | 1109 | 0.2193 | 1138 | 0.0112 |
| 1081 | 0.0101 | 1110 | 0.0231 | 1139 | 0.0127 |
| 1082 | 0.0282 | 1111 | 0.0091 | 1140 | 0.0254 |
| 1083 | 0.0157 | 1112 | 0.0471 | 1141 | 0.0182 |
| 1084 | 0.0209 | 1113 | 0.0939 | 1142 | 0.0248 |
| 1085 | 0.0154 | 1114 | 0.0437 | 1143 | 0.0214 |
| 1086 | 0.0310 | 1115 | 0.0043 | 1144 | 0.0140 |
| 1087 | 0.0332 | 1116 | 0.0089 | 1145 | 0.0112 |
| 1088 | 0.0071 | 1117 | 0.0066 | 1146 | 0.0137 |
| 1089 | 0.0052 | 1118 | 0.0376 | 1147 | 0.0132 |
| 1090 | 0.0083 | 1119 | 0.0302 | 1148 | 0.0178 |
| 1091 | 0.0068 | 1120 | 0.0136 | 1149 | 0.0152 |
| 1092 | 0.0113 | 1121 | 0.0081 | 1150 | 0.0160 |
| 1093 | 0.0146 | 1122 | 0.0038 | 1151 | 0.0061 |
| 1094 | 0.0061 | 1123 | 0.0093 | 1152 | 0.0118 |
| 1095 | 0.0115 | 1124 | 0.0077 | 1153 | 0.0129 |
| 1096 | 0.0131 | 1125 | 0.0099 | 1154 | 0.0156 |
| 1155 | 0.0086 | 1184 | 0.0440 | 1212 | 0.3006 |
| 1156 | 0.0151 | 1185 | 0.0502 | 1213 | 0.2759 |
| 1157 | 0.0046 | 1186 | 0.1084 | 1214 | 0.0772 |
| 1158 | 0.0503 | 1187 | 0.0377 | 1215 | 0.0108 |
| 1159 | 0.0464 | 1188 | 0.0205 | 1216 | 0.2651 |
| 1160 | 0.1008 | 1189 | 0.0124 | 1217 | 0.2380 |
| 1161 | 0.0814 | 1190 | 0.0180 | 1218 | 0.1395 |
| 1162 | 0.0592 | 1191 | 0.0074 | 1219 | 0.2019 |
| 1163 | 0.0152 | 1192 | 0.0175 | 1220 | 0.1817 |
| 1164 | 0.0089 | 1193 | 0.0156 | 1221 | 0.1208 |
| 1165 | 0.0218 | 1194 | 0.0079 | 1222 | 0.5912 |
| 1166 | 0.0571 | 1195 | 0.0153 | 1223 | 0.4196 |
| 1167 | 0.0245 | 1196 | 0.0093 | 1224 | 0.0279 |
| 1168 | 0.0148 | 1197 | 0.0189 | 1225 | 0.0487 |
| 1169 | 0.0606 | 1198 | 0.0196 | 1226 | 0.0806 |
| 1170 | 0.0540 | 1199 | 0.0180 | 1227 | 0.0339 |
| 1171 | 0.0412 | 1200 | 0.0281 | 1228 | 0.0169 |
| 1172 | 0.0274 | 1201 | 0.0160 | 1229 | 0.0152 |
| 1173 | 0.0215 | 1202 | 0.0160 | 1230 | 0.1282 |
| 1174 | 0.0234 | 1203 | 0.0215 | 1231 | 0.0302 |
| 1175 | 0.0488 | 1204 | 0.0247 | 1232 | 0.0341 |
| 1176 | 0.0156 | 1205 | 0.0247 | 1233 | 0.0667 |
| 1177 | 0.0270 | 1206 | 0.0069 | 1234 | 0.0316 |
| 1178 | 0.0124 | 1207 | 0.0206 | 1235 | 0.0095 |
| 1179 | 0.0789 | 1208 | 0.0791 | 1236 | 0.0545 |
| 1180 | 0.0918 | 1209 | 0.0179 | 1237 | 0.1803 |
| 1181 | 0.0485 | 1209 | 0.0179 | 1238 | 0.0149 |
| 1182 | 0.0930 | 1210 | 0.0094 | 1239 | 0.0105 |
| 1183 | 0.0941 | 1211 | 0.3683 | 1240 | 0.0207 |
| 1241 | 0.0233 | 1270 | 0.0619 | 1299 | 0.0116 |
| 1242 | 0.0198 | 1271 | 0.0391 | 1300 | 0.0215 |
| 1243 | 0.0280 | 1272 | 0.0403 | 1301 | 0.0269 |
| 1244 | 0.0203 | 1273 | 0.3271 | 1302 | 0.0176 |
| 1245 | 0.0182 | 1274 | 0.0301 | 1303 | 0.0267 |
| 1246 | 0.0319 | 1275 | 0.2232 | 1304 | 0.0446 |
| 1247 | 0.0041 | 1276 | 0.1416 | 1305 | 0.0248 |
| 1248 | 0.0180 | 1277 | 0.0246 | 1306 | 0.0239 |
| 1249 | 0.0295 | 1278 | 0.0334 | 1307 | 0.0413 |
| 1250 | 0.0028 | 1279 | 0.0393 | 1308 | 0.0070 |
| 1251 | 0.0118 | 1280 | 0.0293 | 1309 | 0.0127 |
| 1252 | 0.0228 | 1281 | 0.0221 | 1310 | 0.0306 |
| 1253 | 0.0166 | 1282 | 0.0072 | 1311 | 0.2439 |
| 1254 | 0.0125 | 1283 | 0.0159 | 1312 | 0.0274 |
| 1255 | 0.2063 | 1284 | 0.0169 | 1313 | 0.0282 |
| 1256 | 0.5024 | 1285 | 0.0292 | 1314 | 0.0070 |
| 1257 | 0.2261 | 1286 | 0.0192 | 1315 | 0.0099 |
| 1258 | 0.0190 | 1287 | 0.0090 | 1316 | 0.0977 |
| 1259 | 0.0149 | 1288 | 0.0147 | 1317 | 0.0365 |
| 1260 | 0.0104 | 1289 | 0.0324 | 1318 | 0.0598 |
| 1261 | 0.0166 | 1290 | 0.0201 | 1319 | 0.0420 |
| 1262 | 0.0089 | 1291 | 0.0227 | 1320 | 0.0150 |
| 1263 | 0.0127 | 1292 | 0.0969 | 1321 | 0.1420 |
| 1264 | 0.0318 | 1293 | 0.0358 | 1322 | 0.0197 |
| 1265 | 0.0441 | 1294 | 0.0116 | 1323 | 0.0994 |
| 1266 | 0.0266 | 1295 | 0.0112 | 1324 | 0.0342 |
| 1267 | 0.0228 | 1296 | 0.0487 | 1325 | 0.0281 |
| 1268 | 0.0178 | 1297 | 0.0333 | 1326 | 0.0198 |
| 1269 | 0.0192 | 1298 | 0.0164 | 1327 | 0.0156 |
| 1328 | 0.0240 | 1357 | 0.0205 | 1386 | 0.1369 |
| 1329 | 0.0153 | 1358 | 0.0456 | 1387 | 0.4010 |
| 1330 | 0.0141 | 1359 | 0.0232 | 1388 | 0.6603 |
| 1331 | 0.0123 | 1360 | 0.0086 | 1389 | 0.2270 |
| 1332 | 0.0145 | 1361 | 0.0161 | 1390 | 0.0273 |
| 1333 | 0.0673 | 1362 | 0.0251 | 1391 | 0.0258 |
| 1334 | 0.1167 | 1363 | 0.0253 | 1392 | 0.0625 |
| 1335 | 0.0590 | 1364 | 0.0495 | 1393 | 0.0331 |
| 1336 | 0.1980 | 1365 | 0.0597 | 1394 | 0.1428 |
| 1337 | 0.0099 | 1366 | 0.1278 | 1395 | 0.0565 |
| 1338 | 0.0108 | 1367 | 0.0556 | 1396 | 0.3044 |
| 1339 | 0.0280 | 1368 | 0.0181 | 1397 | 0.1232 |
| 1340 | 0.0520 | 1369 | 0.0120 | 1398 | 0.0855 |
| 1341 | 0.0091 | 1370 | 0.0139 | 1399 | 0.0688 |
| 1342 | 0.0180 | 1371 | 0.0275 | 1400 | 0.3055 |
| 1343 | 0.0392 | 1372 | 0.0280 | 1401 | 0.0639 |
| 1344 | 0.0695 | 1373 | 0.0317 | 1402 | 0.2035 |
| 1345 | 0.1177 | 1374 | 0.0174 | 1403 | 0.0050 |
| 1346 | 0.1133 | 1375 | 0.0318 | 1404 | 0.0261 |
| 1347 | 0.3015 | 1376 | 0.0458 | 1405 | 0.0236 |
| 1348 | 0.0394 | 1377 | 0.0310 | 1406 | 0.0511 |
| 1349 | 0.0158 | 1378 | 0.0948 | 1407 | 0.0899 |
| 1350 | 0.1186 | 1379 | 0.1746 | 1408 | 0.0289 |
| 1351 | 0.0381 | 1380 | 0.1188 | 1409 | 0.0237 |
| 1352 | 0.0837 | 1381 | 0.0274 | 1410 | 0.0162 |
| 1353 | 0.0163 | 1382 | 0.1104 | 1411 | 0.0097 |
| 1354 | 0.0422 | 1383 | 0.1008 | 1412 | 0.2073 |
| 1355 | 0.0051 | 1384 | 0.1927 | 1413 | 0.0181 |
| 1356 | 0.0146 | 1385 | 0.1001 | 1414 | 0.0030 |
| 1415 | 0.1722 | 2009 | 0.0583 | 2038 | 0.0296 |
| 1416 | 0.3105 | 2010 | 0.0348 | 2039 | 0.8790 |
| 1417 | 0.3786 | 2011 | 0.0883 | 2040 | 0.0087 |
| 1418 | 0.0371 | 2012 | 0.0210 | 2041 | 0.0132 |
| 1419 | 0.1860 | 2013 | 0.0741 | 2042 | 0.0193 |
| 1420 | 0.1158 | 2014 | 0.0427 | 2043 | 0.0174 |
| 1421 | 0.2404 | 2015 | 0.0094 | 2044 | 0.0670 |
| 1422 | 0.0202 | 2016 | 0.0388 | 2045 | 0.0269 |
| 1423 | 0.0281 | 2017 | 0.0243 | 2046 | 0.0255 |
| 1424 | 0.0277 | 2018 | 0.0737 | 2047 | 0.0358 |
| 1425 | 0.1572 | 2019 | 0.0249 | 2048 | 0.0186 |
| 1426 | 0.0181 | 2020 | 0.0305 | 2049 | 0.0182 |
| 1427 | 0.1109 | 2021 | 0.0489 | 2050 | 0.0258 |
| 1428 | 0.4293 | 2022 | 0.1290 | 2051 | 0.1086 |
| 1429 | 0.1431 | 2023 | 0.0474 | 2052 | 0.0791 |
| 1430 | 0.0427 | 2024 | 0.0383 | 2053 | 0.0662 |
| 1431 | 0.9624 | 2025 | 0.0498 | 2054 | 0.0143 |
| 1432 | 0.1053 | 2026 | 0.2374 | 2055 | 0.1008 |
| 1433 | 0.7978 | 2027 | 0.0386 | 2056 | 0.0200 |
| 1434 | 0.9154 | 2028 | 0.0099 | 2057 | 0.1985 |
| 2000 | 0.0147 | 2029 | 0.0109 | 2058 | 0.0599 |
| 2001 | 0.1230 | 2030 | 0.0132 | 2059 | 0.0342 |
| 2002 | 0.0654 | 2031 | 0.0094 | 2060 | 0.0780 |
| 2003 | 0.2895 | 2032 | 0.0104 | 2061 | 0.7768 |
| 2004 | 0.1810 | 2033 | 0.0192 | 2062 | 0.0381 |
| 2005 | 0.2228 | 2034 | 0.0169 | 2063 | 0.0650 |
| 2006 | 0.1371 | 2035 | 0.0219 | 2064 | 0.0211 |
| 2007 | 0.0306 | 2036 | 0.0439 | 2065 | 0.0173 |
| 2008 | 0.0284 | 2037 | 0.0290 | 2066 | 0.1099 |
| 2067 | 0.0352 | 2096 | 0.0118 | 2125 | 0.0115 |
| 2068 | 0.1060 | 2097 | 0.0948 | 2126 | 0.0084 |
| 2069 | 0.0245 | 2098 | 0.0240 | 2127 | 0.0104 |
| 2070 | 0.0974 | 2099 | 0.0170 | 2128 | 0.0089 |
| 2071 | 0.0202 | 2100 | 0.0217 | 2129 | 0.0112 |
| 2072 | 0.0254 | 2101 | 0.0146 | 2130 | 0.0118 |
| 2073 | 0.0440 | 2102 | 0.3366 | 2131 | 0.0191 |
| 2074 | 0.0099 | 2103 | 0.0296 | 2132 | 0.0082 |
| 2075 | 0.0155 | 2104 | 0.0141 | 2133 | 0.0234 |
| 2076 | 0.0543 | 2105 | 0.0150 | 2134 | 0.0320 |
| 2077 | 0.0141 | 2106 | 0.0080 | 2135 | 0.0132 |
| 2078 | 0.0122 | 2107 | 0.0219 | 2136 | 0.1430 |
| 2079 | 0.0113 | 2108 | 0.0244 | 2137 | 0.0304 |
| 2080 | 0.1098 | 2109 | 0.0108 | 2138 | 0.0142 |
| 2081 | 0.2058 | 2110 | 0.0170 | 2139 | 0.0109 |
| 2082 | 0.1686 | 2111 | 0.0299 | 2140 | 0.0157 |
| 2083 | 0.2051 | 2112 | 0.0385 | 2141 | 0.0132 |
| 2084 | 0.0095 | 2113 | 0.0461 | 2142 | 0.0116 |
| 2085 | 0.0098 | 2114 | 0.1020 | 2143 | 0.0613 |
| 2086 | 0.0185 | 2115 | 0.2689 | 2144 | 0.0193 |
| 2087 | 0.0507 | 2116 | 0.0637 | 2145 | 0.0143 |
| 2088 | 0.0128 | 2117 | 0.0303 | 2146 | 0.0127 |
| 2089 | 0.0123 | 2118 | 0.0143 | 2147 | 0.0104 |
| 2090 | 0.0482 | 2119 | 0.0241 | 2148 | 0.0541 |
| 2091 | 0.1195 | 2120 | 0.0176 | 2149 | 0.0177 |
| 2092 | 0.0728 | 2121 | 0.0194 | 2150 | 0.0141 |
| 2093 | 0.0210 | 2122 | 0.0160 | 2151 | 0.0085 |
| 2094 | 0.0096 | 2123 | 0.0116 | 2152 | 0.0199 |
| 2095 | 0.0095 | 2124 | 0.0170 | 2153 | 0.0167 |
| 2154 | 0.0110 | 2183 | 0.0192 | 2212 | 0.0161 |
| 2155 | 0.0066 | 2184 | 0.0302 | 2213 | 0.0144 |
| 2156 | 0.0165 | 2185 | 0.0402 | 2214 | 0.0163 |
| 2157 | 0.0210 | 2186 | 0.1584 | 2215 | 0.0092 |
| 2158 | 0.0200 | 2187 | 0.0062 | 2216 | 0.0139 |
| 2159 | 0.0178 | 2188 | 0.0121 | 2217 | 0.0115 |
| 2160 | 0.0122 | 2189 | 0.0289 | 2218 | 0.0333 |
| 2161 | 0.1218 | 2190 | 0.0517 | 2219 | 0.0062 |
| 2162 | 0.0078 | 2191 | 0.0134 | 2220 | 0.0101 |
| 2163 | 0.0176 | 2192 | 0.0076 | 2221 | 0.0116 |
| 2164 | 0.0119 | 2193 | 0.0087 | 2222 | 0.0106 |
| 2165 | 0.0154 | 2194 | 0.0396 | 2223 | 0.0109 |
| 2166 | 0.0247 | 2195 | 0.0803 | 2224 | 0.0057 |
| 2167 | 0.0244 | 2196 | 0.2014 | 2225 | 0.0097 |
| 2168 | 0.0090 | 2197 | 0.0940 | 2226 | 0.0267 |
| 2169 | 0.0126 | 2198 | 0.0730 | 2227 | 0.0340 |
| 2170 | 0.0086 | 2199 | 0.1323 | 2228 | 0.0520 |
| 2171 | 0.0099 | 2200 | 0.0206 | 2229 | 0.0048 |
| 2172 | 0.0130 | 2201 | 0.0152 | 2230 | 0.0130 |
| 2173 | 0.0156 | 2202 | 0.0313 | 2231 | 0.0033 |
| 2174 | 0.0164 | 2203 | 0.0095 | 2232 | 0.0148 |
| 2175 | 0.0279 | 2204 | 0.0203 | 2233 | 0.0097 |
| 2176 | 0.0202 | 2205 | 0.0141 | 2234 | 0.0134 |
| 2177 | 0.0259 | 2206 | 0.0837 | 2235 | 0.0200 |
| 2178 | 0.0085 | 2207 | 0.0099 | 2236 | 0.0131 |
| 2179 | 0.0112 | 2208 | 0.0247 | 2237 | 0.0150 |
| 2180 | 0.0163 | 2209 | 0.0156 | 2238 | 0.0366 |
| 2181 | 0.0211 | 2210 | 0.0100 | 2239 | 0.0060 |
| 2182 | 0.0123 | 2211 | 0.0085 | 2240 | 0.0063 |
| 2241 | 0.0102 | 2270 | 0.8020 | 2299 | 0.1295 |
| 2242 | 0.1143 | 2271 | 0.2603 | 2300 | 0.0156 |
| 2243 | 0.0260 | 2272 | 0.2306 | 2301 | 0.1507 |
| 2244 | 0.0386 | 2273 | 0.3211 | 2302 | 0.0282 |
| 2245 | 0.0250 | 2274 | 0.0855 | 2303 | 0.0460 |
| 2246 | 0.0161 | 2275 | 0.2819 | 2304 | 0.0313 |
| 2247 | 0.0049 | 2276 | 0.1743 | 2305 | 0.0199 |
| 2248 | 0.0110 | 2277 | 0.0187 | 2306 | 0.0143 |
| 2249 | 0.0122 | 2278 | 0.0342 | 2307 | 0.0138 |
| 2250 | 0.0158 | 2279 | 0.0211 | 2308 | 0.0211 |
| 2251 | 0.1688 | 2280 | 0.1035 | 2309 | 0.0279 |
| 2252 | 0.1267 | 2281 | 0.0507 | 2310 | 0.0121 |
| 2253 | 0.0119 | 2282 | 0.0353 | 2311 | 0.0213 |
| 2254 | 0.1834 | 2283 | 0.0205 | 2312 | 0.0087 |
| 2255 | 0.0631 | 2284 | 0.0236 | 2313 | 0.0101 |
| 2256 | 0.0988 | 2285 | 0.0489 | 2314 | 0.0217 |
| 2257 | 0.1992 | 2286 | 0.0274 | 2315 | 0.0097 |
| 2258 | 0.7646 | 2287 | 0.0179 | 2316 | 0.0113 |
| 2259 | 0.0114 | 2288 | 0.0129 | 2317 | 0.0089 |
| 2260 | 0.0285 | 2289 | 0.0075 | 2318 | 0.0078 |
| 2261 | 0.0258 | 2290 | 0.0180 | 2319 | 0.0156 |
| 2262 | 0.1541 | 2291 | 0.0289 | 2320 | 0.0071 |
| 2263 | 0.3342 | 2292 | 0.0179 | 2321 | 0.0058 |
| 2264 | 0.2816 | 2293 | 0.0202 | 2322 | 0.0095 |
| 2265 | 0.4267 | 2294 | 0.0199 | 2323 | 0.0256 |
| 2266 | 0.3339 | 2295 | 0.0103 | 2324 | 0.0128 |
| 2267 | 0.1977 | 2296 | 0.0346 | 2325 | 0.0300 |
| 2268 | 0.4761 | 2297 | 0.0085 | 2326 | 0.0413 |
| 2269 | 0.0119 | 2298 | 0.0247 | 2327 | 0.0130 |
| 2328 | 0.0195 | 2357 | 0.0667 | 2386 | 0.0347 |
| 2329 | 0.1955 | 2358 | 0.0166 | 2387 | 0.0150 |
| 2330 | 0.0381 | 2359 | 0.0263 | 2388 | 0.0083 |
| 2331 | 0.1601 | 2360 | 0.0269 | 2389 | 0.0213 |
| 2332 | 0.3431 | 2361 | 0.0183 | 2390 | 0.0093 |
| 2333 | 0.0235 | 2362 | 0.0402 | 2391 | 0.0275 |
| 2334 | 0.0228 | 2363 | 0.0219 | 2392 | 0.0296 |
| 2335 | 0.0474 | 2364 | 0.0363 | 2393 | 0.0406 |
| 2336 | 0.0067 | 2365 | 0.0179 | 2394 | 0.0157 |
| 2337 | 0.0058 | 2366 | 0.1269 | 2395 | 0.0115 |
| 2338 | 0.0055 | 2367 | 0.0671 | 2396 | 0.0141 |
| 2339 | 0.0088 | 2368 | 0.0066 | 2397 | 0.0196 |
| 2340 | 0.0239 | 2369 | 0.0231 | 2398 | 0.0166 |
| 2341 | 0.0417 | 2370 | 0.0358 | 2399 | 0.0141 |
| 2342 | 0.0748 | 2371 | 0.0268 | 2400 | 0.0110 |
| 2343 | 0.0517 | 2372 | 0.0078 | 2401 | 0.0944 |
| 2344 | 0.0440 | 2373 | 0.0138 | 2402 | 0.0517 |
| 2345 | 0.0675 | 2374 | 0.6288 | 2403 | 0.0479 |
| 2346 | 0.0231 | 2375 | 0.0158 | 2404 | 0.0409 |
| 2347 | 0.0093 | 2376 | 0.0209 | 2405 | 0.0753 |
| 2348 | 0.0301 | 2377 | 0.0129 | 2406 | 0.1586 |
| 2349 | 0.0325 | 2378 | 0.0155 | 2407 | 0.0661 |
| 2350 | 0.0056 | 2379 | 0.0389 | 2408 | 0.2279 |
| 2351 | 0.0222 | 2380 | 0.0146 | 2409 | 0.1093 |
| 2352 | 0.0145 | 2381 | 0.0338 | 2410 | 0.1081 |
| 2353 | 0.0215 | 2382 | 0.0067 | 2411 | 0.0381 |
| 2354 | 0.0214 | 2383 | 0.0153 | 2412 | 0.0129 |
| 2355 | 0.0453 | 2384 | 0.0371 | 2413 | 0.0165 |
| 2356 | 0.0347 | 2385 | 0.0261 | 2414 | 0.0450 |
| 2415 | 0.0412 | 2444 | 0.0404 | 2473 | 0.0321 |
| 2416 | 0.5696 | 2445 | 0.0465 | 2474 | 0.0249 |
| 2417 | 0.0323 | 2446 | 0.2388 | 2475 | 0.0189 |
| 2418 | 0.0468 | 2447 | 0.0382 | 2476 | 0.0144 |
| 2419 | 0.0524 | 2448 | 0.1284 | 2477 | 0.0245 |
| 2420 | 0.0354 | 2449 | 0.2716 | 2478 | 0.0252 |
| 2421 | 0.0146 | 2450 | 0.3188 | 2479 | 0.0196 |
| 2422 | 0.0249 | 2451 | 0.0139 | 2480 | 0.0174 |
| 2423 | 0.0142 | 2452 | 0.0122 | 2481 | 0.0146 |
| 2424 | 0.1081 | 2453 | 0.0131 | 2482 | 0.0207 |
| 2425 | 0.0156 | 2454 | 0.0111 | 2483 | 0.0126 |
| 2426 | 0.0360 | 2455 | 0.0084 | 2484 | 0.2937 |
| 2427 | 0.0307 | 2456 | 0.0125 | 2485 | 0.0115 |
| 2428 | 0.2586 | 2457 | 0.0412 | 2486 | 0.3051 |
| 2429 | 0.2170 | 2458 | 0.0221 | 2487 | 0.0095 |
| 2430 | 0.0342 | 2459 | 0.0451 | 2488 | 0.0346 |
| 2431 | 0.1399 | 2460 | 0.0149 | 2489 | 0.1359 |
| 2432 | 0.9726 | 2461 | 0.0141 | 2490 | 0.0169 |
| 2433 | 0.0352 | 2462 | 0.0565 | 2491 | 0.0191 |
| 2434 | 0.0335 | 2463 | 0.0693 | 2492 | 0.0311 |
| 2435 | 0.0255 | 2464 | 0.0460 | 2493 | 0.0048 |
| 2436 | 0.0314 | 2465 | 0.0702 | 2494 | 0.0092 |
| 2437 | 0.0349 | 2466 | 0.0548 | 2495 | 0.1075 |
| 2438 | 0.0168 | 2467 | 0.1611 | 2496 | 0.0671 |
| 2439 | 0.0346 | 2468 | 0.0223 | 2497 | 0.0325 |
| 2440 | 0.0227 | 2469 | 0.0253 | 2498 | 0.0362 |
| 2441 | 0.0338 | 2470 | 0.0087 | 2499 | 0.0780 |
| 2442 | 0.0379 | 2471 | 0.0093 | 2500 | 0.0195 |
| 2443 | 0.0236 | 2472 | 0.0205 | 2501 | 0.2534 |
| 2502 | 0.1477 | 2531 | 0.0010 | 2560 | 0.0021 |
| 2503 | 0.0122 | 2532 | 0.0014 | 2561 | 0.0241 |
| 2504 | 0.0132 | 2533 | 0.0023 | 2562 | 0.0021 |
| 2505 | 0.0129 | 2534 | 0.0017 | 2563 | 0.0046 |
| 2506 | 0.0103 | 2535 | 0.0036 | 2564 | 0.0011 |
| 2507 | 0.3934 | 2536 | 0.4352 | 2565 | 0.0033 |
| 2508 | 0.0406 | 2537 | 0.0171 | 2566 | 0.0045 |
| 2509 | 0.0130 | 2538 | 0.0124 | 2567 | 0.0022 |
| 2510 | 0.0181 | 2539 | 0.3496 | 2568 | 0.0228 |
| 2511 | 0.0161 | 2540 | 0.0090 | 2569 | 0.0453 |
| 2512 | 0.0216 | 2541 | 0.1818 | 2570 | 0.0027 |
| 2513 | 0.0144 | 2542 | 0.0024 | 2571 | 0.1080 |
| 2514 | 0.0168 | 2543 | 0.0320 | 2572 | 0.0115 |
| 2515 | 0.0031 | 2544 | 0.3614 | 2573 | 0.0097 |
| 2516 | 0.0207 | 2545 | 0.0131 | 2574 | 0.0044 |
| 2517 | 0.0300 | 2546 | 0.0117 | 2575 | 0.0296 |
| 2518 | 0.0143 | 2547 | 0.0149 | 2576 | 0.1033 |
| 2519 | 0.0131 | 2548 | 0.0196 | 2577 | 0.0469 |
| 2520 | 0.0080 | 2549 | 0.0054 | 2578 | 0.0222 |
| 2521 | 0.0134 | 2550 | 0.0028 | 2579 | 0.0026 |
| 2522 | 0.0257 | 2551 | 0.0175 | 2580 | 0.0068 |
| 2523 | 0.0071 | 2552 | 0.0187 | 2581 | 0.0092 |
| 2524 | 0.0080 | 2553 | 0.0048 | 2582 | 0.0213 |
| 2525 | 0.0079 | 2554 | 0.0086 | 2583 | 0.0375 |
| 2526 | 0.0169 | 2555 | 0.0202 | 2584 | 0.1628 |
| 2527 | 0.0018 | 2556 | 0.0412 | 2585 | 0.0025 |
| 2528 | 0.0086 | 2557 | 0.0007 | 2586 | 0.0141 |
| 2529 | 0.0044 | 2558 | 0.0116 | 2587 | 0.0135 |
| 2530 | 0.0024 | 2559 | 0.0286 | 2588 | 0.0042 |
| 2589 | 0.0356 | 2618 | 0.2101 | 2647 | 0.4117 |
| 2590 | 0.1853 | 2619 | 0.0439 | 2648 | 0.1033 |
| 2591 | 0.0031 | 2620 | 0.0720 | 2649 | 0.1376 |
| 2592 | 0.0896 | 2621 | 0.0224 | 2650 | 0.1611 |
| 2593 | 0.0090 | 2622 | 0.0839 | 2651 | 0.0485 |
| 2594 | 0.0057 | 2623 | 0.0994 | 2652 | 0.0470 |
| 2595 | 0.0117 | 2624 | 0.0770 | 2653 | 0.3208 |
| 2596 | 0.0027 | 2625 | 0.1897 | 2654 | 0.2402 |
| 2597 | 0.0033 | 2626 | 0.0090 | 2655 | 0.5842 |
| 2598 | 0.0028 | 2627 | 0.1644 | 2656 | 0.0856 |
| 2599 | 0.0282 | 2628 | 0.2467 | 2657 | 0.0883 |
| 2600 | 0.0055 | 2629 | 0.0306 | 2658 | 0.0236 |
| 2601 | 0.0627 | 2630 | 0.0095 | 2659 | 0.0114 |
| 2602 | 0.1915 | 2631 | 0.0155 | 2660 | — |
| 2603 | 0.4050 | 2632 | 0.0249 | 2661 | — |
| 2604 | 0.4281 | 2633 | 0.2069 | 2662 | — |
| 2605 | 0.6149 | 2634 | 0.1881 | 2663 | — |
| 2606 | 0.0489 | 2635 | 0.1050 | 2664 | 0.0023 |
| 2607 | 0.0351 | 2636 | 0.1356 | 2665 | 0.0012 |
| 2608 | 0.1678 | 2637 | 0.0242 | 2666 | 0.0007 |
| 2609 | 0.0962 | 2638 | 0.0091 | 2667 | 0.0003 |
| 2610 | 0.3505 | 2639 | 0.0168 | 2668 | 0.0026 |
| 2611 | 0.0600 | 2640 | 0.0397 | 2669 | 0.0024 |
| 2612 | 0.0629 | 2641 | 0.0389 | 2670 | 0.0066 |
| 2613 | 0.3827 | 2642 | 0.0744 | 2671 | 0.0301 |
| 2614 | 0.1902 | 2643 | 0.0399 | 2672 | 0.1998 |
| 2615 | 0.0641 | 2644 | 0.0166 | 2673 | 0.0903 |
| 2616 | 0.4266 | 2645 | 0.1142 | 2674 | 0.0213 |
| 2617 | 0.0201 | 2646 | 0.0345 | 2675 | 0.0182 |
| 2676 | 0.5032 | 2681 | 0.0473 | 2686 | 0.2569 |
| 2677 | 0.0028 | 2682 | 0.0580 | 2687 | 0.1426 |
| 2678 | 0.0162 | 2683 | 0.0056 | 2688 | 0.0545 |
| 2679 | 0.0122 | 2684 | 0.0044 | 2689 | 0.0061 |
| 2680 | 0.0106 | 2685 | 0.0036 | | |

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A compound of formula (I): 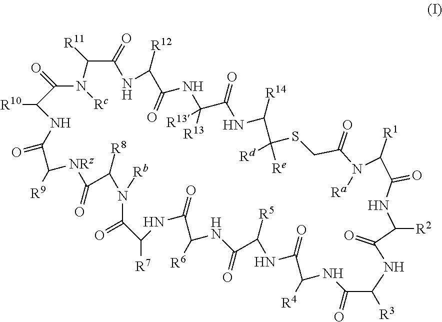
or a pharmaceutically acceptable salt thereof, wherein:
R¹ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₆alkyl, carboxyC₁-C₆alkyl, cyanoC₁-C₆alkyl, C₃-C₆cycloalkylcarbonylaminoC₁-C₆alkyl, guanidinylC₁-C₆alkyl, heteroarylC₁-C₆alkyl, heterocyclylC₁-C₆alkyl, hydroxyC₁-C₆alkyl, and methoxyC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, trifluoromethoxy, and trifluoromethyl, and wherein the heteroaryl part of the heteroarylC₁-C₆alkyl is optionally substituted with one, two, three, four, or five C₁-C₆alkyl groups;
R² is selected from arylC₁-C₆alkyl, guanidinylC₁-C₆alkyl, and heteroarylC₁-C₆alkyl; wherein the aryl part of the arylC₁-C₆alkyl is optionally substituted with one, two, three, four, or five groups independently selected from aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl; and wherein the heteroaryl part of the heteroarylC₁-C₆alkyl is optionally substituted with one, two, three, four, or five carboxyC₁-C₆alkyl groups;
R³ is carboxyC₁-C₆alkyl;
R⁴ is arylC₁-C₆alkyl or heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, cyano, halo, hydroxy, and trifluoromethyl; and wherein the heteroaryl part of the heteroarylC₁-C₆alkyl is optionally substituted with one, two, three, four, or five groups independently selected from C₁-C₆alkoxy, C₁-C₆alkyl, cyano, halo, hydroxy, and trifluoromethyl;
R⁵ is selected from C₂-C₆alkenyl, C₁-C₆alkyl, aminocarbonylC₁-C₆alkyl, aminocarbonylaminoC₁-C₆alkyl, C₅-C₆aryl, arylC₁-C₆alkyl, carboxyC₁-C₆alkyl, C₃-C₆cycloalkyl, and heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl;
R⁶ is biarylC₁-C₆alkyl; wherein the biaryl part of the biarylC₁-C₆alkyl is optionally substituted with one, two, three, four, or five groups independently selected from C₁-C₆alkoxy, C₁-C₆alkoxyC₁-C₆alkyl, C₁-C₆alkylcarbonylamino, aminocarbonyl, arylC₁-C₆alkoxy, cyanoC₁-C₆alkyl, halo, heteroaryl, trifluoromethoxy, and trifluoromethyl;
R⁷ is selected from C₁-C₆alkyl, C₁-C₆alkylaminoC₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₆alkyl, carboxyC₁-C₆alkyl, C₃-C₆cycloalkylcarbonylaminoC₁-C₆alkyl, guanidinylC₁-C₆alkyl, C₁-C₆haloalkylcarbonylaminoC₁-C₆alkyl, and hydroxyC₁-C₆alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl;
R⁸ is selected from C₁-C₆alkyl, aminocarbonylC₁-C₆alkyl, carboxyC₁-C₆alkyl, guanidinylC₁-C₆alkyl, and hydroxyC₁-C₆alkyl;
R^{z} is hydrogen and R⁹ is selected from C₁-C₆alkyl, aminoC₁-C₆alkyl, and C₃-C₆cycloalkylC₁-C₆alkyl; or
R⁹ and R^{z}, together with the atoms to which they are attached, form a proline ring;
R¹⁰ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, carboxyC₁-C₆alkyl, and heteroarylC₁-C₆alkyl;
R¹¹ is C₁-C₆alkyl or C₃-C₈cycloalkylC₁-C₆alkyl;
R¹² is selected from C₁-C₆alkyl, C₁-C₆alkylcarbonylaminoC₁-C₆alkyl, aminoC₁-C₆alkyl, haloC₁-C₆alkyl, heteroarylC₁-C₆alkyl, and hydroxyC₁-C₆alkyl;
R^{13′} is hydrogen and R¹³ is selected from C₁-C₆alkyl, C₁-C₆alkylcarbonylaminoC₁-C₆alkyl, C₂-C₆alkynyloxyC₁-C₆alkyl, aminoC₁-C₆alkyl, aminocarbonylC₁-C₆alkyl, aminocarbonylaminoC₁-C₆alkyl, arylC₁-C₆alkyl, azidoC₁-C₆alkyl, carboxyC₁-C₆alkyl, guanidinylC₁-C₆alkyl, hydroxyC₁-C₆alkyl, and heteroarylC₁-C₆alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl; and wherein the heteroaryl part of the heteroarylC₁-C₆alkyl is optionally substituted with one, two, three, four, or five groups independently selected from C₁-C₆alkyl, halo, haloarylcarbonylaminoC₁-C₆alkyl, and hydroxy; or
R¹³ and R^{13′}, together with the carbon atom to which they are attached, form a cyclopropyl ring;
R¹⁴ is —C(O)NR^{14′}CR¹⁵R^{15′}R^{15″}, —C(O)NH(CH₂)ⱼPh(CH₂)ⱼC(O)NHCHR¹⁷R^{17′}, —C(O)NH(CH₂)ⱼcyclopropyl(CH₂)ⱼC(O)NHCHR¹⁷R^{17′}, or —C(O)NR⁵⁰R⁵¹, wherein j is 0, 1, or 2, and wherein:
R⁵⁰ and R⁵¹, together with the nitrogen atom to which they are attached, form a piperazine ring, wherein the ring is further substituted with one —(CH₂)ⱼC(O)NHCHR¹⁷R^{17′} group;
R^{14′} is hydrogen or C₁-C₆alkyl, or R¹⁵ and R^{14′}, together with the atoms to which they are attached, form a morpholine, piperazine, or piperidine ring;
R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is —(CH₂)ₘCO₂H CH₂O((CH₂)₂O)ₙCH₂C(O)NHCHR¹⁶R^{16′}, or —C(O)NHCHR¹⁶R^{16′}; wherein:
R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{16′} is —(CH₂)ₘCO₂H, —CH₂O((CH₂)₂O)ₙCH₂C(O)NR⁷⁵CR^{17″}R¹⁷R^{17′},
-Ph(CH₂)ⱼC(O)NHCHR¹⁷R^{17′} or —(CH₂)ⱼC(O)NHCHR¹⁷R^{17′}; wherein:
R⁷⁵ is hydrogen;
R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R⁷ and R⁷⁵, together with the atoms to which they are attached, form a pyrrolidine ring;
R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
R^{17″} is hydrogen, or R^{17″} and R¹⁷ form a C₃-C₈ cycloalkyl ring; wherein:
R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{18′} is —(CH₂)ₘCO₂H, —(CH₂)ₘC(O)NR¹⁹R^{19′}, or CH₂O((CH₂)₂O)ₙCH₂C(O)NHCHR¹⁹R^{19′}; wherein:
R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
R^{19′} is —(CH₂)ₘC(O)NR¹⁹R^{19′}, —(CH₂)ₘCO₂H, or —CH₂O((CH₂)₂O)ₙCH₂C(O)NHCHR²⁰R^{20′}; wherein:
R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{20′} is —(CH₂)ₘCO₂H or —(CH₂)ₘC(O)NR²¹R^{21′}; wherein:
R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{21′} is —(CH₂)ₘCO₂H or —(CH₂)ₘC(O)NR²²R^{22′}; wherein:
R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{22′} is —(CH₂)ₘCO₂H; wherein:
m is a integer from 1 to 10;
n is 1, 2, or 3; and
j is 0, 1, or 2;
R^{a} is hydrogen or C₁-C₆alkyl;
R^{b} is C₁-C₆alkyl, or aminoC₁-C₆alkyl;
R^{c} is hydrogen or C₁-C₆alkyl;
R^{d} is hydrogen or C₁-C₆alkyl; and
R^{e} is hydrogen or C₁-C₆alkyl.

2. The compound of claim 1, or the pharmaceutically acceptable salt thereof, wherein R¹ is selected from C₁-C₄alkyl, aminoC₁-C₄alkyl, aminocarbonylaminopropyl, aminocarbonylmethyl, arylC₁-C₂alkyl, tert-butylcarbonylaminoethyl, carboxyethyl, cyanoC₁-C₄alkyl, cyclopropylcarbonylaminoethyl, guanidinylC₃-C₄alkyl, heteroarylC₁-C₆alkyl, heterocyclylmethyl, hydroxyethyl, hydroxymethyl, methoxyethyl, and methoxymethyl; wherein the aryl part of the arylC₁-C₂alkyl is optionally substituted with one, two, or three groups independently selected from amino, aminoC₂-C₆alkoxy, aminoC₁-C₆alkyl, aminocarbonyl, carboxy, carboxymethoxy cyano, halo, hydroxy, methoxy, trifluoromethoxy, and trifluoromethyl.

3. The compound of claim 1 or claim 2, or the pharmaceutically acceptable salt thereof, wherein R² is selected from aminoC₁-C₄alkyl, aminocarbonylmethyl, arylC₁-C₂alkyl, butyl, tert-butylcarbonylaminoC₂-C₄alkyl, guanidinylC₃-C₄alkyl, heteroarylC₁-C₆alkyl, hydroxymethyl, hydroxyethyl, and isopentyl; wherein the aryl part of the arylC₁-C₂alkyl is optionally substituted with one, two, or three groups independently selected from aminocarbonyl, carboxy, carboxymethoxy, carboxymethyl, cyano, fluoro, hydroxy, and trifluoromethyl.

4. The compound of any one of claims 1 to 3, or the pharmaceutically acceptable salt thereof, wherein R³ is carboxyC₁-C₄alkyl.

5. The compound of claim 4, or the pharmaceutically acceptable salt thereof, wherein R³ is carboxymethyl.

6. The compound of any one of claims 1 to 5, or the pharmaceutically acceptable salt thereof, wherein R⁴ is arylC₁-C₆alkyl or heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkyl, cyano, halo, and trifluoromethyl.

7. The compound of claim 6, wherein R⁴ is benzyl, optionally substituted with one, two, three, four, or five groups independently selected from C₁-C₆alkyl, cyano, halo, and trifluoromethyl.

8. The compound of claim 6, wherein R⁴ is indolylC₁-C₆alkyl.

9. The compound of any one of claims 1 to 8, or the pharmaceutically acceptable salt thereof, wherein R⁵ is selected from C₁-C₆alkyl, C₅-C₆aryl, arylC₁-C₆alkyl, carboxyC₂-C₃alkyl, C₃-C₆cycloalkyl, and heteroarylC₁-C₆alkyl; wherein the aryl part of the arylC₁-C₆alkyl is optionally substituted with one, two, three, four, or five groups independently selected from C₁-C₆alkyl, amino, aminoC₂-C₆alkoxy, aminoC₁-C₆alkyl, aminocarbonyl, carboxy, carboxyC₁-C₆alkoxy, carboxyC₁-C₆alkyl, cyano, halo, hydroxy, nitro, and trifluoromethyl.

10. The compound of claim 9, or the pharmaceutically acceptable salt thereof, wherein R⁵ is arylmethyl or isopropyl; wherein the aryl part of the arylmethyl is optionally substituted with one, two, three, four, or five groups independently selected from aminocarbonyl, carboxy, carboxymethoxy, and hydroxy.

11. The compound of claim 10, wherein R⁵ is benzyl where the phenyl part is optionally substituted with one, two, three, four, or five groups selected from aminocarbonyl, carboxy, carboxymethoxy, and hydroxy.

12. The compound of any one of claims 1 to 11, or the pharmaceutically acceptable salt thereof, wherein R⁶ is biarylC₁-C₆alkyl; wherein the biaryl part of the biarylC₁-C₆alkyl is optionally substituted with one, two, or three fluoro groups.

13. The compound of claim 12, or the pharmaceutically acceptable salt thereof, wherein R⁶ is biphenylC₁-C₆alkyl.

14. The compound of any one of claims 1 to 13, or the pharmaceutically acceptable salt thereof, wherein R⁷ is selected from C₁-C₆alkyl, C₁-C₆alkylcarbonylaminoC₁-C₆alkyl, aminocarbonylC₁-C₆alkyl, aminocarbonylaminoC₁-C₆alkyl, arylC₁-C₆alkyl, C₃-C₆cycloalkylcarbonylaminoC₁-C₆alkyl, guanidinylC₁-C₆alkyl, and hydroxyC₁-C₃alkyl; wherein the aryl part of the arylC₁-C₆alkyl is optionally substituted with one, two, three, four, or five groups independently selected from carboxy, carboxyC₁-C₆alkoxy, carboxyC₁-C₆alkyl, hydroxy, and trifluoromethyl.

15. The compound of claim 14, or the pharmaceutically acceptable salt thereof, wherein R⁷ is selected from aminocarbonylaminopropyl, aminocarbonylethyl, arylmethyl, isopentyl, isopropyl, and methylcarbonylaminobutyl; wherein the aryl part of the arylmethyl is optionally substituted with one, two, three, four, or five groups independently selected from carboxy and carboxymethoxy.

16. The compound of any one of claims 1 to 15, or the pharmaceutically acceptable salt thereof, wherein R⁸ is selected from of C₁-C₆alkyl, aminocarbonylC₁-C₆alkyl, carboxyC₁-C₆alkyl, guanidinylC₁-C₆alkyl, and hydroxymethyl.

17. The compound of claim 16, or the pharmaceutically acceptable salt thereof, wherein R⁸ is methyl.

18. The compound of any one of claims 1 to 17, or the pharmaceutically acceptable salt thereof, wherein R⁹ is C₁-C₆alkyl or aminoC₁-C₆alkyl.

19. The compound of claim 18, or the pharmaceutically acceptable salt thereof, wherein R⁹ is isobutyl.

20. The compound of any one of claims 1 to 19, or the pharmaceutically acceptable salt thereof, wherein R¹⁰ is aminoC₁-C₆alkyl or heteroarylC₁-C₆alkyl.

21. The compound of claim 20, wherein the heteroaryl in heteroarylC₁-C₆alkyl is imidazolyl.

22. The compound of any one of claims 1 to 21, or the pharmaceutically acceptable salt thereof, wherein R¹¹ is C₁-C₆alkyl or cyclohexylmethyl.

23. The compound of any one of claims 1 to 22, or the pharmaceutically acceptable salt thereof, wherein R¹² is selected from C₁-C₄alkyl, aminoC₁-C₆alkyl, and hydroxyC₁-C₆alkyl.

24. The compound of any one of claims 1 to 23, or the pharmaceutically acceptable salt thereof, wherein R¹³ is selected from aminobutyl, aminocarbonylethyl, aminoethyl, aminomethyl, carboxyethyl, hydroxyC₁-C₃alkyl imidazolylmethyl, methylcarbonylaminobutyl, and guanidinylpropyl.

25. The compound of claim 1, or the pharmaceutically acceptable salt thereof, wherein:
R¹ is selected from aminoC₁-C₄alkyl, aminocarbonylaminopropyl, aminocarbonylmethyl, arylC₁-C₂alkyl, butyl, tert-butylcarbonylaminoethyl, carboxyethyl, cyanomethyl, cyclopropycarbonylaminoethyl, ethyl, guanidinylC₃-C₄alkyl, hydroxyethyl, hydroxymethyl, isobutyl, methoxyethyl, methoxymethyl, methyl, heteroarylC₁-C₆alkyl, heterocyclylC₁-C₆alkyl, and propyl; wherein the aryl part of the arylC₁-C₂alkyl is optionally substituted with one, two, or three groups independently selected from amino, aminoC₂-C₆alkoxy, aminoC₁-C₆alkyl, aminocarbonyl, carboxy, carboxymethoxy, cyano, halo, hydroxy, methoxy, trifluoromethoxy, and trifluoromethyl;
R² is selected from arylC₁-C₂alkyl, guanidinylC₃-C₄alkyl, and heteroarylC₁-C₆alkyl; wherein the aryl part of the arylC₁-C₂alkyl is optionally substituted with one, two, or three groups independently selected from aminocarbonyl, carboxy, carboxyC₁-C₆alkoxy, cyano, hydroxy, trifluoromethoxy, and trifluoromethyl; wherein the heteroaryl part of the heteroarylC₁-C₆alkyl is optionally substituted with one, two, or three carboxymethoxy groups;
R³ is carboxymethyl;
R⁴ is arylmethyl or heteroarylmethyl; and wherein the aryl part of the arylmethyl is optionally substituted with one, two, three, four, or five substituents independently selected from chloro, cyano, fluoro, methyl, and trifluoromethyl;
R⁵ is selected from C₂-C₆alkenyl, C₁-C₆alkyl, aminocarbonylC₁-C₆alkyl, aminocarbonylaminoethyl, aminocarbonylaminopropyl, arylmethyl, carboxyethyl, carboxypropyl, C₃-C₆cycloalkyl, heteroarylC₁-C₆alkyl, and phenyl; wherein the aryl part of the arylmethyl is optionally substituted with one, two, three, four, or five groups independently selected from amino, aminocarbonyl, carboxy, carboxymethoxy chloro, cyano, fluoro, hydroxy, methoxy, methyl, and trifluoromethyl;
R⁶ is biarylC₁-C₆alkyl, wherein the biaryl part of the biarylC₁-C₆alkyl is optionally substituted with one, two, three, four, or five groups independently selected from chloro and fluoro;
R⁷ is selected from C₁-C₆alkyl, C₁-C₆alkylaminoC₁-C₆alkyl, aminoC₁-C₆alkyl, aminocarbonylaminopropyl, C₁-C₆alkylcarbonylaminoC₁-C₆alkyl, aminocarbonylethyl, arylmethyl, carboxyC₁-C₆alkyl, C₁-C₆cycloalkylcarbonylaminoC₁-C₆alkyl, guanidinylpropyl, C₁-C₆haloalkylcarbonylaminoC₁-C₆alkyl, hydroxymethyl, and methylcarbonylaminobutyl; wherein the aryl part of the arylmethyl is optionally substituted with one, two, three, four, or five groups independently selected from carboxy, carboxyC₁-C₆alkoxy, hydroxy, and trifluoromethyl;
R⁸ is selected from aminocarbonylmethyl, aminocarbonylethyl, carboxyethyl hydroxymethyl, and methyl;
R^{z} is hydrogen and R⁹ is selected from C₁-C₆alkyl, cyclopropylC₁-C₆alkyl, and cyclobutylmethyl; or
R⁹ and R^{z}, together with the atoms to which they are attached, form a proline ring;
R¹⁰ is selected from aminobutyl, aminoethyl, aminomethyl, aminopropyl, aminocarbonylmethyl, aminocarbonylaminopropyl, carboxymethyl, carboxyethyl, and imidazolylmethyl;
R¹¹ is isobutyl or cyclohexylmethyl;
R¹² is selected from C₁-C₄alkyl, C₁-C₆alkylcarbonylaminoC₁-C₆alkyl, aminoC₁-C₄alkyl, aminocarbonylaminopropyl, haloC₁-C₆alkyl, hydroxyC₁-C₄alkyl, and imidazolylmethyl;
R¹³ is selected from C₁-C₆alkylcarbonylaminoC₁-C₆alkyl, C₂-C₆alkynyloxyC₁-C₆alkyl aminoC₁-C₄alkyl, aminocarbonylC₁-C₃alkyl, aminocarbonylaminopropyl, azidoC₁-C₆alkyl, carboxyC₁-C₃alkoxy, carboxyC₁-C₃alkyl, hydroxyC₁-C₄alkyl, imidazolylmethyl, methylcarbonylaminobutyl, and triazolylmethyl optionally substituted with a haloarylcarbonylaminomethyl or guanidinylpropyl group;
R¹⁴ is —C(O)NR^{14′}CR¹⁵R^{15′}R^{15″}, wherein:
R^{14′} is hydrogen, C₁-C₆alkyl, or R¹⁵ and R^{14′}, together with the atoms to which they are attached, form a piperazine ring;
R¹⁵ is selected from hydrogen, C₁-C₁₆alkyl, aminoC₁-C₆alkyl, aminocarbonylC₁-C₆alkyl, carboxy, carboxyC₁-C₆alkyl, guanidinylC₁-C₆alkyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is hydrogen, carboxy, or —C(O)NHCHR¹⁶R^{16′}; wherein:
R¹⁶ is hydrogen, C₂-C₁₆alkyl, aminoC₁-C₆alkyl, aminocarbonylaminoC₁-C₆alkyl, or carboxyC₁-C₆alkyl;
and
R^{16′} is hydrogen, carboxy, —((CH₂)₂O)ₙCH₂C(O)NHCHR¹⁷R^{17′}, —(NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, aminoC₁-C₆alkyl, carboxy, or carboxyC₁-C₆alkyl; and
R^{17′} is (CH₂)ₘC(O)NHR¹⁸R^{18′};
m is 0, 1, 2 or 3; wherein:
R¹⁸ is C₁₀-C₁₂alkyl; and
R^{18′} is carboxy;
R^{a} is hydrogen or methyl;
R^{b} is ethyl or methyl;
R^{c} is hydrogen;
R^{d} is hydrogen; and
R^{e} is hydrogen.

26. The compound of claim 25, or the pharmaceutically acceptable salt thereof, wherein R¹ is selected from aminoC₁-C₄alkyl, aminocarbonylmethyl, butyl, tert-butylcarbonylaminoC₂-C₄alkyl, cyanomethyl, C₃-C₆cycloalkylcarbonylaminoC₁-C₆alkyl, guanidinylC₃-C₄alkyl, heteroarylC₁-C₆alkyl, heterocyclylC₁-C₆alkyl, hydroxyethyl, hydroxymethyl, isobutyl, methoxymethyl, and phenylC₁-C₂alkyl; wherein the phenyl part of the phenylC₁-C₂alkyl is optionally substituted with one, two, or three groups independently selected from aminocarbonyl, carboxy, carboxymethoxy cyano, fluoro, methoxy, and trifluoromethyl;
R² is selected from arylmethyl, guanidinylC₃-C₄alkyl, and heteroarylC₁-C₆alkyl; wherein the aryl part of the arylmethyl is optionally substituted with one, two, or three groups independently selected from aminocarbonyl, carboxy, carboxyC₁-C₆alkoxy, cyano, hydroxy, and trifluoromethyl;
R³ is carboxymethyl;
R⁴ is arylmethyl or heteroarylmethyl; wherein the aryl part of the arylmethyl is optionally substituted with one, two, three, four, or five groups independently selected from halo, methyl, and trifluoromethyl;
R⁵ is selected from arylmethyl, carboxyethyl, carboxypropyl, cyclohexyl, cyclopropyl, ethyl, heteroarylmethyl, isobutyl, isopropyl, and phenyl; wherein the aryl part of the arylmethyl is optionally substituted with one, two, three, four, or five groups independently selected from amino, aminocarbonyl, carboxy, carboxymethoxy fluoro, hydroxy, and trifluoromethyl;
R⁶ is biarylC₁-C₆alkyl, and wherein the biaryl part of the biarylC₁-C₆alkyl is optionally substituted with one, two, three, four, or five fluoro groups;
R⁷ is selected from aminocarbonylaminopropyl, aminocarbonylethyl, aminomethyl, arylmethyl, tert-butylcarbonylaminobutyl, carboxyethyl, C₃-C₆cycloalkylcarbonylaminoC₁-C₆alkyl, guanidinylpropyl, C₁-C₆haloalkylcarbonylaminoC₁-C₆alkyl, hydroxyethyl, isobutyl, isopropyl, and methylcarbonylaminobutyl; wherein the aryl part of the arylmethyl is optionally substituted with one, two, three, four, or five groups independently selected from carboxy, carboxyC₁-C₆alkyl, hydroxy, and trifluoromethyl;
R⁸ is methyl;
R⁹ is selected from cyclobutylmethyl, isobutyl, and methyl;
R¹⁰ is selected from aminocarbonylmethyl, aminoethyl, aminopropyl, carboxypropyl, and imidazolylmethyl;
R¹¹ is cyclohexylmethyl or isobutyl;
R¹² is selected from C₁-C₄alkyl, aminocarbonylaminopropyl, fluoroC₁-C₆alkyl, and hydroxyC₁-C₂alkyl;
R¹³ is selected from acetylaminobutyl, C₂-C₆alkynyloxymethyl, aminobutyl, aminocarbonylaminopropyl, aminocarbonylethyl, aminocarbonylmethyl, aminoethyl, aminomethyl, aminopropyl, carboxyethyl, carboxymethyl, carboxypropyl, ethyl, guanidinylpropyl, hydroxybutyl, hydroxyethyl, hydroxymethyl, imidazolylmethyl, and isopropyl;
R¹⁴ is —C(O)NR^{14′}CR¹⁵R^{15′}R^{15″}, wherein:
R^{14′} is hydrogen, C₁-C₆alkyl, or R¹⁵ and R^{14′}, together with the atoms to which they are attached, form a piperazine ring;
R¹⁵ is selected from hydrogen, methyl, C₁₀alkyl, aminomethyl, aminoethyl, aminopropyl, aminobutyl, carboxyethyl, aminocarbonylmethyl, hydroxymethyl, hydroxyethyl, guanidinylpropyl, and imidazolylmethyl;
R^{15′} is hydrogen or R¹⁵ and R^{15′}, together with the atoms to which they are attached, form a C₃-C₈cycloalkyl ring; and
R^{15″} is hydrogen, carboxy, or —C(O)NHCHR¹⁶R^{16′}; wherein:
R¹⁶ is hydrogen, C₂-C₁₆alkyl, aminomethyl, aminoethyl, aminocarbonylaminoC₁-C₆alkyl, or carboxyC₁-C₆alkyl; and
R^{16′} is hydrogen, C₁-C₆alkyl, carboxy, —((CH₂)₂O)ₙCH₂C(O)NHCHR¹⁷R^{17′}, (NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, carboxy, aminoethyl, carboxyC₁-C₆alkyl; and
R^{17′} is (CH₂)ₘC(O)NHR¹⁸R^{18′};
m is 0, 1, 2 or 3; wherein:
R¹⁸ is C₉-C₁₂alkyl; and
R^{18′}is carboxy;
R^{a} is hydrogen or methyl;
R^{b} is ethyl or methyl;
R^{c} is hydrogen;
R^{d} is hydrogen; and
R^{e} is hydrogen.

27. The compound of claim 26, or the pharmaceutically acceptable salt thereof, wherein:
R¹ is selected from aminoethyl, benzyl, butyl, guanidinylpropyl, hydroxyethyl, imidazolylC₁-C₂alkyl, morpholinylmethyl, and pyridinylC₁-C₂alkyl; wherein the phenyl part of the benzyl is optionally substituted with one, two, or three groups independently selected from aminocarbonyl, carboxy, carboxymethoxy, cyano, fluoro, and trifluoromethyl;
R² is benzyl or pyridinylC₁-C₆alkyl; wherein the phenyl part of the benzyl is optionally substituted with one, two, or three groups independently selected from carboxy and carboxyC₁-C₆alkoxy;
R³ is carboxymethyl;
R⁴ is benzyl or indolylmethyl; and wherein the phenyl part of the benzyl is optionally substituted with one or more groups independently selected from methyl and trifluoromethyl;
R⁵ is benzyl, isobutyl, or isopropyl, wherein the phenyl part of the benzyl is optionally substituted with one, two, three, four, or five groups independently selected from aminocarbonyl, carboxy, carboxymethoxy and hydroxy;
R⁶ is biphenylC₁-C₆alkyl;
R⁷ is selected from aminocarbonylaminopropyl, aminocarbonylethyl, benzyl, isopropyl, isobutyl, and methylcarbonylaminobutyl, wherein the phenyl part of the benzyl is optionally substituted with one, two, three, four, or five groups independently selected from carboxy and carboxymethoxy;
R⁸ is methyl;
R⁹ is isobutyl;
R¹⁰ is aminoethyl or imidazolylmethyl;
R¹¹ is cyclohexylmethyl;
R¹² is C₁-C₄alkyl or hydroxyC₁-C₂alkyl;
R¹³ is selected from aminobutyl, aminocarbonylethyl, aminoethyl, aminomethyl, carboxyethyl, carboxymethyl, guanidinylpropyl, hydroxyC₁-C₃alkyl imidazolylmethyl, and methylcarbonylaminobutyl;
R¹⁴ is —C(O)NR^{14′}CR¹⁵R^{15′}R^{15″}, wherein:
R^{14′} is hydrogen, C₁-C₆alkyl, or R¹⁵ and R^{14′}, together with the atoms to which they are attached, form a piperazinyl ring;
R¹⁵ is selected from hydrogen, methyl, C₁₀alkyl, and aminoethyl;
R^{15′} is hydrogen or R¹⁵ and R^{15′}, together with the atoms to which they are attached, form a cyclopropyl ring; and
R^{15′} is hydrogen, carboxy, or —C(O)NHCHR¹⁶R^{16′}; wherein:
R¹⁶ is hydrogen, C₂-C₁₆alkyl, aminoC₁-C₆alkyl, aminocarbonylaminoC₁-C₆alkyl, or carboxyC₁-C₆alkyl; and
R^{16′} is hydrogen, C₁-C₆alkyl, carboxy, —((CH₂)₂O)ₙCH₂C(O)NHCHR¹⁷R^{17′}, —(NHCH₂)ₒCH₂C(O)NHCHR¹⁷R^{17′}, or —C(O)NHCHR¹⁷R^{17′}; wherein:
n is 1, 2, or 3;
o is 1, 2, or 3;
R¹⁷ is hydrogen, carboxy, aminoC₁-C₆alkyl, carboxyC₁-C₆alkyl; and
R^{17′} is —(CH₂)ₘC(O)NHR¹⁸R^{18′};
m is 0, 1, 2 or 3; wherein:
R¹⁸ is C₁₀alkyl; and
R¹⁸ is carboxy;
R^{a} is hydrogen or methyl;
R^{b} is methyl;
R^{c} is hydrogen;
R^{d} is hydrogen; and
R^{e} is hydrogen.

28. The compound of claim 1, or the pharmaceutically acceptable salt thereof, wherein the compound is selected from the compounds listed in Table 3.

29. A pharmaceutical composition comprising a compound of any one of claims 1 to 28, or a pharmaceutically acceptable salt thereof.

30. A method of enhancing, stimulating, and/or increasing an immune response in a subject in need thereof, wherein the method comprises administering to the subject a therapeutically effective amount of a compound of any one of claims 1 to 28 or a pharmaceutically acceptable salt thereof.

31. A method of blocking the interaction of PD-1 with PD-L1 in a subject, wherein the method comprises administering to the subject a therapeutically effective amount of a compound of any one of claims 1 to 28 or a pharmaceutically acceptable salt thereof.
